(19) United States
(12) Patent Application Publication
(10) Pub. No.: US 2026/0224743 A1
(43) Pub. Date: Aug. 6, 2026

(54) TRAC AND B2M MODULATING COMPOSITIONS AND METHODS

(71) Applicant: Flagship Pioneering Innovations VI, LLC, Cambridge, MA (US)

(72) Inventors: Anne Helen Bothmer, Cambridge, MA (US); Aamir Mir, Medford, MA (US); Cecilia Giovanna Silvia Cotta-Ramusino, Cambridge, MA (US); Giulia Schiroli, Boston, MA (US)

(21) Appl. No.: 19/145,316

(22) PCT Filed: Jan. 5, 2024
(86) PCT No.: PCT/US2024/010523

§ 371 Date: Jul. 2, 2025

Related U.S. Application Data
(60) Provisional application No. 63/478,925, filed on Jan. 7, 2023.
(60) Provisional application No. 63/592,181, filed on Oct. 22, 2023.
(60) Provisional application No. 63/598,370, filed on Nov. 13, 2023.

(51) Int. Cl. A61K 48/00; C12N 9/12; C12N 9/22; C12N 15/11; C12N 15/86

(52) U.S. Cl. CPC A61K 48/005; C12N 9/1276; C12N 9/226; C12N 15/111; C12N 15/86; C07K 2319/09; C07K 2319/80; C12N 2310/20; C12N 2750/14143; C12Y 207/07049

(57) ABSTRACT

The disclosure provides, e.g., compositions, systems, and methods for targeting, editing, modifying, or manipulating a host cell's genome at one or more locations in a DNA sequence in a cell, tissue, or subject. Heterologous gene modifying systems for disrupting the TRAC gene and/or the B2M gene are described.

### CROSS REFERENCE TO RELATED APPLICATIONS

This application claims the benefit of U.S. Provisional Application No. 63/478,925, filed Jan. 7, 2023, U.S. Provisional Application No. 63/592,181, filed Oct. 22, 2023, and U.S. Provisional Application No. 63/598,370, filed Nov. 13, 2023. The contents of the aforementioned applications are hereby incorporated by reference in their entirety.

### SEQUENCE LISTING

The instant application contains a Sequence Listing which has been submitted electronically in XML format compliant with WIPO Standard ST.26 and is hereby incorporated by reference in its entirety. Said XML copy, created on Dec. 14, 2023, is named V2065-7028WO_SL.xml and is 44,117,218 bytes in size.

### BACKGROUND

Integration of a nucleic acid of interest into a genome occurs at low frequency and with little site specificity, in the absence of a specialized protein to promote the insertion event. Some existing approaches, like CRISPR/Cas9, are more suited for small edits that rely on host repair pathways, and are less effective at integrating longer sequences. Other existing approaches, like Cre/loxP, require a first step of inserting a loxP site into the genome and then a second step of inserting a sequence of interest into the loxP site. There is a need in the art for improved compositions (e.g., proteins and nucleic acids) and methods for inserting, altering, or deleting sequences of interest in a genome.

T cell therapeutics show promise for treating a number of diseases that are difficult to target with traditional small molecule drugs. For example, chimeric antigen receptor (CAR) T cells have been shown to target immune system responses to cancer cells. Expression of endogenous T cell receptor components in T cells engineered as therapeutics can lead to graft-versus-host-disease in an allogeneic transplantation setting. To achieve highly potent CAR-T cells, it may be desirable to eliminate the endogenous T cell receptor components. There is a need in the art for improved compositions and methods for disrupting expression of endogenously expressed T cell proteins.

### SUMMARY OF THE INVENTION

This disclosure relates to novel compositions, systems, and methods for altering a genome at one or more locations in a host cell, tissue, or subject, in vivo or in vitro. In particular, the invention features compositions, systems, and methods for inserting, altering, or deleting sequences of interest in a host genome. For example, the disclosure provides systems that are capable of modulating (e.g., inserting, altering, or deleting sequences of interest) the TRAC gene and/or B2M gene activity and methods of administering one or more such systems to alter a genomic sequence at nucleotide TRAC and/or B2M genetic locus to disrupt the TRAC gene and/or B2M gene.

In one aspect, the disclosure relates to a system for modifying DNA to disrupt a human TRAC gene comprising (a) a nucleic acid encoding a heterologous gene modifying polypeptide capable of target primed reverse transcription, the polypeptide comprising (i) a reverse transcriptase domain and (ii) a Cas9 nickase that binds DNA and has endonuclease activity, and (b) a template RNA comprising (i) a gRNA spacer that is complementary to a first portion of the human TRAC gene, (ii) a gRNA scaffold that binds the polypeptide, (iii) a heterologous object sequence comprising a mutation region to install a mutation, and (iv) a primer binding site (PBS) sequence comprising at least 3, 4, 5, 6, 7, or 8 bases of 100% homology to a target DNA strand at the 3′ end of the template RNA. The template RNA sequence may comprise a sequence described herein, e.g., in Table 1A, 3A, or 4A. In some embodiments, the mutation leads to a loss of TRAC mRNA. In some embodiments, the mutation leads to an inactive TRAC protein.

In one aspect, the disclosure relates to a system for modifying DNA to disrupt a human B2M gene comprising (a) a nucleic acid encoding a heterologous gene modifying polypeptide capable of target primed reverse transcription, the polypeptide comprising (i) a reverse transcriptase domain and (ii) a Cas9 nickase that binds DNA and has endonuclease activity, and (b) a template RNA comprising (i) a gRNA spacer that is complementary to a first portion of the human B2M gene, (ii) a gRNA scaffold that binds the polypeptide, (iii) a heterologous object sequence comprising a mutation region to install a mutation, and (iv) a primer binding site (PBS) sequence comprising at least 3, 4, 5, 6, 7, or 8 bases of 100% homology to a target DNA strand at the 3′ end of the template RNA. The template RNA sequence may comprise a sequence described herein, e.g., in Table 1B, 3B, or 4B. In some embodiments, the mutation leads to a loss of B2M mRNA. In some embodiments, the mutation leads to an inactive B2M protein.

In one aspect, the disclosure relates to a system for modifying DNA to disrupt a human TRAC gene and B2M gene comprising (a) a nucleic acid encoding a heterologous gene modifying polypeptide capable of target primed reverse transcription, the polypeptide comprising (i) a reverse transcriptase domain and (ii) a Cas9 nickase that binds DNA and has endonuclease activity, (b) a template RNA comprising (i) a gRNA spacer that is complementary to a first portion of the human TRAC gene, (ii) a gRNA scaffold that binds the polypeptide, (iii) a heterologous object sequence comprising a mutation region to install a mutation in the TRAC gene, and (iv) a primer binding site (PBS) sequence comprising at least 3, 4, 5, 6, 7, or 8 bases of 100% homology to a target DNA strand at the 3′ end of the template RNA (optionally, the template RNA sequence may comprise a sequence described herein, e.g., in Table 1A, 3A, or 4a); and (c) a second template RNA comprising (i) a gRNA spacer that is complementary to a first portion of the human B2M gene, (ii) a gRNA scaffold that binds the polypeptide, (iii) a heterologous object sequence comprising a mutation region to install a mutation in the B2M gene, and (iv) a primer binding site (PBS) sequence comprising at least 3, 4, 5, 6, 7, or 8 bases of 100% homology to a target DNA strand at the 3′ end of the template RNA (optionally, the template RNA sequence may comprise a sequence described herein, e.g., in Table 1B, 3B, or 4B). In some embodiments, the mutations lead to a loss of TRAC and B2M mRNAs. In some embodiments, the mutations lead to inactive TRAC and B2M proteins.

The gRNA spacer may comprise at least 15 bases of 100% homology to the target DNA at the 5′ end of the template RNA. The template RNA may further comprise a PBS sequence comprising at least 5 bases of at least 80% homology to the target DNA strand. The template RNA may comprise one or more chemical modifications.

The domains of the heterologous gene modifying polypeptide may be joined by a peptide linker. The polypeptide may comprise one or more peptide linkers. The heterologous gene modifying polypeptide may further comprise a nuclear localization signal. The polypeptide may comprise more than one nuclear localization signal, e.g., multiple adjacent nuclear localization signals or one or more nuclear localization signals in different regions of the polypeptide, e.g., one or more nuclear localization signals in the N-terminus of the polypeptide and one or more nuclear localization signals in the C-terminus of the polypeptide. The nucleic acid encoding the heterologous gene modifying polypeptide may encode one or more intein domains.

Introduction of the system into a target cell may result in insertion of at least 1, 2, 3, 4, 5, 10, 15, 20, 25, 30, 35, 40, 45, 50, 60, 70, 80, 90, 100, 150, 200, 250, 300, 350, 400, 500, or 1000 base pairs of exogenous DNA. Introduction of the system into a target cell may result in deletion, wherein the deletion is less than 2, 3, 4, 5, 10, 50, or 100 base pairs of genomic DNA upstream or downstream of the insertion. Introduction of the system into a target cell may result in substitution, e.g., substitution of 1, 2, or 3 nucleotides, e.g., consecutive nucleotides.

The heterologous object sequence may be at least 5, 10, 25, 50, 100, 150, 200, 250, 300, 400, 500, 600, or 700 base pairs.

In one aspect, the disclosure relates to a pharmaceutical composition comprising the system described above and a pharmaceutically acceptable excipient or carrier, wherein the pharmaceutically acceptable excipient or carrier is selected from the group consisting of a plasmid vector, a viral vector, a vesicle, and a lipid nanoparticle. In one aspect, the disclosure relates to a pharmaceutical composition comprising the system described above and multiple pharmaceutically acceptable excipients or carriers, wherein the pharmaceutically acceptable excipients or carriers are selected from the group consisting of a plasmid vector, a viral vector, a vesicle, and a lipid nanoparticle, e.g., where the system described above is delivered by two distinct excipients or carriers, e.g., two lipid nanoparticles, two viral vectors, or one lipid nanoparticle and one viral vector. The viral vector may be an adeno-associated virus (AAV).

In one aspect, the disclosure relates to a host cell (e.g., a mammalian cell, e.g., a human cell) comprising the system described above.

In one aspect, the disclosure relates to a method of installing a mutation in the human TRAC gene and/or B2M gene in a cell, tissue or subject, the method comprising administering the system described above to the cell, tissue or subject, wherein optionally the installation of the mutation in the TRAC gene and/or B2M gene comprises an insertion of at least 5 nucleotides or a deletion of at least 5 nucleotides within the TRAC gene and/or B2M gene (e.g., resulting in the formation of a premature stop codon within the TRAC gene and/or B2M gene). The system may be introduced in vivo, in vitro, ex vivo, or in situ. The nucleic acid of (a) may be integrated into the genome of the host cell. In some embodiments, the nucleic acid of (a) is not integrated into the genome of the host cell. In some embodiments, the heterologous object sequence is inserted at only one target site in the host cell genome. The heterologous object sequence may be inserted at two or more target sites in the host cell genome, e.g., at the same corresponding site in two homologous chromosomes or at two different sites on the same or different chromosomes. The heterologous object sequence may encode a mammalian polypeptide, or a fragment or a variant thereof. The components of the system may be delivered on 1, 2, 3, 4, or more distinct nucleic acid molecules. The system may be introduced into a host cell by electroporation or by using at least one vehicle selected from a plasmid vector, a viral vector, a vesicle, and a lipid nanoparticle.

Features of the compositions or methods can include one or more of the following enumerated embodiments.

### Enumerated Embodiments

1. A template RNA comprising, e.g., from 5′ to 3′:
   (i) a gRNA spacer that is complementary to a first portion of the human TRAC gene, wherein the gRNA spacer has a sequence comprising the nucleotides of a gRNA spacer sequence of Table 1A, 2A, or 3A, a sequence having at least 17, 18, 19, or 20 consecutive nucleotides starting with the 3′ end of the gRNA spacer of Table 1A, 2A, or 3A, or a sequence having 1, 2, or 3 substitutions thereto;
   (ii) a gRNA scaffold that binds a heterologous gene modifying polypeptide (e.g., binds the Cas domain of the heterologous gene modifying polypeptide),
   (iii) a heterologous object sequence comprising a mutation region to introduce a mutation (e.g., an insertion or a deletion) into a second portion of the human TRAC gene (wherein optionally the heterologous object sequence comprises, from 5′ to 3′, a post-edit homology region, a mutation region, and a pre-edit homology region), and
   (iv) a primer binding site (PBS) sequence comprising at least 3, 4, 5, 6, 7, or 8 bases with 100% identity to a third portion of the human TRAC gene.
2. The template RNA of embodiment 1, wherein the heterologous object sequence comprises the nucleotides of an RT template sequence from Table 1A, 2A, or 3A, a sequence having at least 5, 6, 7, 8, 9, 10, 11, 12, 13, 14, or 15 nucleotides starting with the 3′ end of the RT template sequence of Table 1A, 2A, or 3A, or a sequence having 1, 2, or 3 substitutions thereto.
3. The template RNA of embodiment 1, wherein the heterologous object sequence comprises the nucleotides of the RT template sequence of Table 1A, 2A, or 3A that corresponds to the gRNA spacer sequence, or a sequence having 1, 2, or 3 substitutions thereto.
4. The template RNA according to any one of embodiments 1-3 wherein the PBS sequence has a sequence comprising the nucleotides of the PBS sequence from the same row of Table 1A, 2A, or 3A as the RT template sequence, a sequence having at least 5, 6, 7, 8, 9, 10, 11, 12, 13, 14, 15, 16, or 17 nucleotides starting with the 5′ end of a PBS sequence from Table 1A, 2A, or 3A, or a sequence having 1, 2, or 3 substitutions thereto.
5. The template RNA according to any one of embodiments 1-3, wherein the PBS sequence has a sequence comprising the nucleotides of a PBS sequence of Table 1A, 2A, or 3A that corresponds to the RT template sequence, or a sequence having 1, 2, or 3 substitutions thereto, the gRNA spacer sequence, or both.
6. The template RNA according to any of embodiments 1-5, wherein the gRNA scaffold comprises a sequence of a gRNA scaffold of any of Tables 1A, 2A, 3A, 12, 14B, or 14C, or a sequence having at least 70%, 75%, 80%, 85%, 90%, 95%, 96%, 97%, 98%, or 99% identity thereto.
7. The template RNA according to any of embodiments 1-5, wherein the gRNA scaffold comprises a sequence of a gRNA scaffold of any of Tables 1A, 2A, 3A, 12, 14B, or 14C that corresponds to the RT template sequence, the gRNA spacer sequence, or both, or a sequence having at least 70%, 75%, 80%, 85%, 90%, 95%, 96%, 97%, 98%, or 99% identity thereto.
8. A template RNA comprising, e.g., from 5′ to 3′:
   (i) a gRNA spacer that is complementary to a first portion of the human TRAC gene,
   (ii) a gRNA scaffold that binds a heterologous gene modifying polypeptide (e.g., binds the Cas domain of the heterologous gene modifying polypeptide),
   (iii) a heterologous object sequence comprising a mutation region to introduce a mutation into a second portion of the human TRAC gene, wherein the heterologous object sequence comprises the nucleotides of an RT template sequence of Table 1A, 2A, or 3A, a sequence having at least 5, 6, 7, 8, 9, 10, 11, 12, 13, 14, or 15 nucleotides starting with the 3′ end of the RT template sequence of Table 1A, 2A, or 3A, or a sequence having 1, 2, or 3 substitutions thereto; and
   (iv) a PBS sequence comprising at least 3, 4, 5, 6, 7, or 8 bases of 100% identity to a third portion of the human TRAC gene.
9. The template RNA of embodiment 8, wherein the gRNA spacer comprises the nucleotides of a gRNA spacer sequence of Table 1A, 2A, or 3A, a sequence having at least 17, 18, 19, or 20 consecutive nucleotides starting with the 3′ end of the gRNA spacer of Table 1A, 2A, or 3A, or a sequence having 1, 2, or 3 substitutions thereto.
10. The template RNA of embodiment 8, wherein the heterologous object sequence comprises the nucleotides of the gRNA spacer sequence of Table 1A, 2A, or 3A that corresponds to the RT template sequence, or a sequence having 1, 2, or 3 substitutions thereto.
11. The template RNA according to any one of embodiments 8-10, wherein the PBS sequence has a sequence comprising the nucleotides of the PBS sequence from the same row of Table 1A, 2A or 3A as the RT template sequence, a sequence having at least 5, 6, 7, 8, 9, 10, 11, 12, 13, 14, 15, 16, or 17 nucleotides starting with the 5′ end of a PBS sequence from Table 1A, 2A, or 3A, or a sequence having 1, 2, or 3 substitutions thereto.
12. The template RNA according to any one of embodiments 8-10, wherein the PBS sequence has a sequence comprising the nucleotides of a PBS sequence of Table 1A, 2A, or 3A that corresponds to the RT template sequence, or a sequence having 1, 2, or 3 substitutions thereto, the gRNA spacer sequence, or both.
13. The template RNA according to any of embodiments 8-12, wherein the gRNA scaffold comprises a sequence of a gRNA scaffold of any of Tables 1A, 2A, 3A, 12, 14B, or 14C, or a sequence having at least 70%, 75%, 80%, 85%, 90%, 95%, 96%, 97%, 98%, or 99% identity thereto.
14. The template RNA according to any of embodiments 8-12, wherein the gRNA scaffold comprises a sequence of a gRNA scaffold of any of Tables 1A, 2A, 3A, 12, 14B, or 14C that corresponds to the RT template sequence, the gRNA spacer sequence, or both, or a sequence having at least 70%, 75%, 80%, 85%, 90%, 95%, 96%, 97%, 98%, or 99% identity thereto.
15. A template RNA comprising, e.g., from 5′ to 3′:
   (i) a gRNA spacer that is complementary to a first portion of the human B2M gene, wherein the gRNA spacer has a sequence comprising the nucleotides of a gRNA spacer sequence of Table 1B, 2B, or 3B, a sequence having at least 17, 18, 19, or 20 consecutive nucleotides starting with the 3′ end of the gRNA spacer of Table 1B, 2B, 3B, or a sequence having 1, 2, or 3 substitutions thereto;
   (ii) a gRNA scaffold that binds a heterologous gene modifying polypeptide (e.g., binds the Cas domain of the heterologous gene modifying polypeptide),
   (iii) a heterologous object sequence comprising a mutation region to introduce a mutation (e.g., an insertion or a deletion) into a second portion of the human B2M gene (wherein optionally the heterologous object sequence comprises, from 5′ to 3′, a post-edit homology region, a mutation region, and a pre-edit homology region), and
   (iv) a primer binding site (PBS) sequence comprising at least 3, 4, 5, 6, 7, or 8 bases with 100% identity to a third portion of the human B2M gene.
16. The template RNA of embodiment 15, wherein the heterologous object sequence comprises the nucleotides of an RT template sequence from Table 1B, 2B, 3B, a sequence having at least 4, 5, 6, 7, 8, 9, 10, 11, 12, 13, 14, or 15 nucleotides starting with the 3′ end of the RT template sequence of Table 1B, 2B, 3B, or a sequence having 1, 2, or 3 substitutions thereto.
17. The template RNA of embodiment 15, wherein the heterologous object sequence comprises the nucleotides of the RT template sequence of Table 1B, 2B, 3B that corresponds to the gRNA spacer sequence, or a sequence having 1, 2, or 3 substitutions thereto.
18. The template RNA according to any one of embodiments 15-17 wherein the PBS sequence has a sequence comprising the nucleotides of the PBS sequence from the same row of Table 1B, 2B, 3B as the RT template sequence, a sequence having at least 5, 6, 7, 8, 9, 10, 11, 12, 13, 14, 15, 16, or 17 nucleotides starting with the 5′ end of a PBS sequence from Table 1B, 2B, 3B, or a sequence having 1, 2, or 3 substitutions thereto.
19. The template RNA according to any one of embodiments 15-17, wherein the PBS sequence has a sequence comprising the nucleotides of a PBS sequence of Table 1B, 2B, 3B that corresponds to the RT template sequence, or a sequence having 1, 2, or 3 substitutions thereto, the gRNA spacer sequence, or both.
20. The template RNA according to any of embodiments 15-19, wherein the gRNA scaffold comprises a sequence of a gRNA scaffold of any of Tables 1B, 2B, 3B, or 12, or a sequence having at least 70%, 75%, 80%, 85%, 90%, 95%, 96%, 97%, 98%, or 99% identity thereto.
21. The template RNA according to any of embodiments 15-19, wherein the gRNA scaffold comprises a sequence of a gRNA scaffold of any of Tables 1B, 2B, 3B, or 12 that corresponds to the RT template sequence, the gRNA spacer sequence, or both, or a sequence having at least 70%, 75%, 80%, 85%, 90%, 95%, 96%, 97%, 98%, or 99% identity thereto.
22. A template RNA comprising, e.g., from 5′ to 3′:
   (i) a gRNA spacer that is complementary to a first portion of the human B2M gene,
   (ii) a gRNA scaffold that binds a heterologous gene modifying polypeptide (e.g., binds the Cas domain of the heterologous gene modifying polypeptide),
   (iii) a heterologous object sequence comprising a mutation region to introduce a mutation into a second portion of the human B2M gene, wherein the heterologous object sequence comprises the nucleotides of an RT template sequence of Table 1B, 2B, or 3B, a sequence having at least 4, 5, 6, 7, 8, 9, 10, 11, 12, 13, 14, or 15 nucleotides starting with the 3′ end of the RT template sequence of Table 1B, 2B, 3B, or a sequence having 1, 2, or 3 substitutions thereto; and
   (iv) a PBS sequence comprising at least 3, 4, 5, 6, 7, or 8 bases of 100% identity to a third portion of the human B2M gene.
23. The template RNA of embodiment 22, wherein the gRNA spacer comprises the nucleotides of a gRNA spacer sequence of Table 1B, 2B, 3B, a sequence having at least 17, 18, 19, or 20 consecutive nucleotides starting with the 3′ end of the gRNA spacer of Table 1B, 2B, or 3B, or a sequence having 1, 2, or 3 substitutions thereto.
24. The template RNA of embodiment 22, wherein the heterologous object sequence comprises the nucleotides of the gRNA spacer sequence of Table 1B, 2B, or 3B that corresponds to the RT template sequence, or a sequence having 1, 2, or 3 substitutions thereto.
25. The template RNA according to any one of embodiments 22-24, wherein the PBS sequence has a sequence comprising the nucleotides of the PBS sequence from the same row of Table 1B, 2B, or 3B as the RT template sequence, a sequence having at least 5, 6, 7, 8, 9, 10, 11, 12, 13, 14, 15, 16, or 17 nucleotides starting with the 5′ end of a PBS sequence from Table 1A, 2A, or 3A, or a sequence having 1, 2, or 3 substitutions thereto.
26. The template RNA according to any one of embodiments 22-24, wherein the PBS sequence has a sequence comprising the nucleotides of a PBS sequence of Table 1B, 2B, or 3B that corresponds to the RT template sequence, or a sequence having 1, 2, or 3 substitutions thereto, the gRNA spacer sequence, or both.
27. The template RNA according to any of embodiments 22-26, wherein the gRNA scaffold comprises a sequence of a gRNA scaffold of any of Tables 1B, 2B, 3B, or 12, or a sequence having at least 70%, 75%, 80%, 85%, 90%, 95%, 96%, 97%, 98%, or 99% identity thereto.
28. The template RNA according to any of embodiments 22-26, wherein the gRNA scaffold comprises a sequence of a gRNA scaffold of any of Tables 1B, 2B, 3B, or 12 that corresponds to the RT template sequence, the gRNA spacer sequence, or both, or a sequence having at least 70%, 75%, 80%, 85%, 90%, 95%, 96%, 97%, 98%, or 99% identity thereto.
29. The template RNA according to any of embodiments 15-28, wherein the template RNA comprises one or more chemical modifications.
30. A template RNA comprising, e.g., from 5′ to 3′:
   (i) a gRNA spacer that is complementary to a first portion of a target DNA;
   (ii) a gRNA scaffold;
   (iii) a heterologous object sequence comprising a mutation region configured to make an insertion of between 1 and 5 nucleotides in the target DNA (wherein optionally the heterologous object sequence comprises, from 5′ to 3′, a post-edit homology region, a mutation region, and a pre-edit homology region), and
   (iv) a primer binding site (PBS) sequence.
31. The template RNA of embodiment 30, wherein, a pre-edit homology region is not present, or is present and has a length of no more than 1 or 2 nucleotides.
32. The template RNA of embodiment 30, wherein the template is designed to introduce the mutation at the protospacer nick site.
33. A heterologous gene modifying system for modifying DNA, comprising:
   (a) a first RNA comprising, from 5′ to 3, (i) a guide RNA sequence that is complementary to a first portion of the human TRAC gene, wherein the guide RNA sequence has a sequence comprising the nucleotides of a spacer sequence of Table 1A, 2A, or 3A, a sequence having at least 17, 18, 19, or 20 consecutive nucleotides starting with the 3′ end of the gRNA spacer of Table 1A, 2A, or 3A, or a sequence having 1, 2, or 3 substitutions thereto; and (ii) a sequence (e.g., a scaffold region) that binds a heterologous gene modifying polypeptide (e.g., binds the Cas domain of the heterologous gene modifying polypeptide), and
   (b) a second RNA comprising (iii) a heterologous object sequence comprising a nucleotide substitution to introduce a mutation into a second portion of the human TRAC gene (wherein optionally the heterologous object sequence comprises, from 5′ to 3′, a post-edit homology region, a mutation region, and a pre-edit homology region), (iv) a primer region comprising at least 5, 6, 7, or 8 bases of 100% identity to a third portion of the human TRAC gene, and (v) an RRS (RNA binding protein recognition sequence) that binds a heterologous gene modifying protein.
34. The heterologous gene modifying system of embodiment 33, wherein the heterologous object sequence comprises the nucleotides of an RT template sequence from Table 1A, 2A, or 3A, a sequence having at least 5, 6, 7, 8, 9, 10, 11, 12, 13, 14, or 15 nucleotides starting with the 3′ end of the RT template sequence of Table 1A, 2A, or 3A, or a sequence having 1, 2, or 3 substitutions thereto.
35. The heterologous gene modifying system of embodiment 33, wherein the heterologous object sequence comprises the nucleotides of the RT template sequence of Table 1A, 2A, or 3A that corresponds to the gRNA spacer sequence, or a sequence having 1, 2, or 3 substitutions thereto.
36 The heterologous gene modifying system of any one of embodiments 33-35, wherein the PBS sequence has a sequence comprising the nucleotides of the PBS sequence from the same row of Table 1A, 2A, or 3A as the RT template sequence, a sequence having at least 5, 6, 7, 8, 9, 10, 11, 12, 13, 14, 15, 16, or 17 nucleotides starting with the 5′ end of a PBS sequence from Table 1A, 2A, or 3A, or a sequence having 1, 2, or 3 substitutions thereto.
37. The heterologous gene modifying system of one of embodiments 33-35, wherein the PBS sequence has a sequence comprising the nucleotides of a PBS sequence of Table 1A, 2A, or 3A that corresponds to the RT template sequence, or a sequence having 1, 2, or 3 substitutions thereto, the gRNA spacer sequence, or both.
38. The heterologous gene modifying system of any one of embodiments 33-37, wherein the gRNA scaffold comprises a sequence of a gRNA scaffold of any of Tables 1A, 2A, 3A, 12, 14B, or 14C, or a sequence having at least 70%, 75%, 80%, 85%, 90%, 95%, 96%, 97%, 98%, or 99% identity thereto.
39. The heterologous gene modifying system of any one of embodiments 33-37, wherein the gRNA scaffold comprises a sequence of a gRNA scaffold of any of Tables 1A, 2A, 3A, 12, 14B, or 14C that corresponds to the RT template sequence, the gRNA spacer sequence, or both, or a sequence having at least 70%, 75%, 80%, 85%, 90%, 95%, 96%, 97%, 98%, or 99% identity thereto.
40. A heterologous gene modifying system for modifying DNA, comprising:
   (a) a first RNA comprising, from 5′ to 3, (i) a guide RNA sequence that is complementary to a first portion of the human TRAC gene, and (ii) a sequence (e.g., a scaffold region) that binds a heterologous gene modifying polypeptide (e.g., binds the Cas domain of the heterologous gene modifying polypeptide), and
   (b) a second RNA comprising (iii) a heterologous object sequence comprising a nucleotide substitution to introduce a mutation into a second portion of the human TRAC gene, wherein the heterologous object sequence comprises the nucleotides of an RT template sequence of Table 1A, 2A, or 3A, a sequence having at least 5, 6, 7, 8, 9, 10, 11, 12, 13, 14, or 15 nucleotides starting with the 3′ end of the RT template sequence of Table 1A, 2A, or 3A, or a sequence having 1, 2, or 3 substitutions thereto, and (iv) a primer region comprising at least 5, 6, 7, or 8 bases of 100% homology to a third portion of the human TRAC gene, and (v) an RRS (RNA binding protein recognition sequence) that binds a heterologous gene modifying protein.
41. The heterologous gene modifying system of embodiment 40, wherein the gRNA spacer comprises the nucleotides of a gRNA spacer sequence of Table 1A, 2A, or 3A, or a sequence having 1, 2, or 3 substitutions thereto.
42. The heterologous gene modifying system of embodiment 40, wherein the heterologous object sequence comprises the nucleotides of the gRNA spacer sequence of Table 1A, 2A, or 3A that corresponds to the RT template sequence, a sequence having at least 5, 6, 7, 8, 9, 10, 11, 12, 13, 14, or 15 nucleotides starting with the 3′ end of the RT template sequence of Table 1A, 2A, or 3A, or a sequence having 1, 2, or 3 substitutions thereto.
43. The heterologous gene modifying system of any one of embodiments 40-42, wherein the PBS sequence has a sequence comprising the nucleotides of the PBS sequence from the same row of Table 1A, 2A, or 3A as the RT template sequence, a sequence having at least 5, 6, 7, 8, 9, 10, 11, 12, 13, 14, 15, 16, or 17 nucleotides starting with the 5′ end of a PBS sequence from Table 1A, 2A, or 3A, or a sequence having 1, 2, or 3 substitutions thereto.
44. The heterologous gene modifying system of any one of embodiments 40-42, wherein the PBS sequence has a sequence comprising the nucleotides of a PBS sequence of Table 1A, 2A, or 3A that corresponds to the RT template sequence, the gRNA spacer sequence, or both, or a sequence having 1, 2, or 3 substitutions thereto.
45. The heterologous gene modifying system of any one of embodiments 40-44, wherein the gRNA scaffold comprises a sequence of a gRNA scaffold of any of Tables 1A, 2A, 3A, 12, 14B, or 14C, or a sequence having at least 70%, 75%, 80%, 85%, 90%, 95%, 96%, 97%, 98%, or 99% identity thereto.
46. The heterologous gene modifying system of any one of embodiments 40-44, wherein the gRNA scaffold comprises a sequence of a gRNA scaffold of any of Tables 1A, 2A, 3A, 12, 14B, or 14C that corresponds to the RT template sequence, the gRNA spacer sequence, or both, or a sequence having at least 70%, 75%, 80%, 85%, 90%, 95%, 96%, 97%, 98%, or 99% identity thereto.
47. A heterologous gene modifying system for modifying DNA, comprising:
   (a) a first RNA comprising, from 5′ to 3, (i) a guide RNA sequence that is complementary to a first portion of the human B2M gene, wherein the guide RNA sequence has a sequence comprising the nucleotides of a spacer sequence of Table 1B, 2B, or 3B, a sequence having at least 17, 18, 19, or 20 consecutive nucleotides starting with the 3′ end of the gRNA spacer of Table 1B, 2B, or 3B, or a sequence having 1, 2, or 3 substitutions thereto; and (ii) a sequence (e.g., a scaffold region) that binds a heterologous gene modifying polypeptide (e.g., binds the Cas domain of the heterologous gene modifying polypeptide), and
   (b) a second RNA comprising (iii) a heterologous object sequence comprising a nucleotide substitution to introduce a mutation into a second portion of the human B2M gene (wherein optionally the heterologous object sequence comprises, from 5′ to 3′, a post-edit homology region, a mutation region, and a pre-edit homology region), (iv) a primer region comprising at least 5, 6, 7, or 8 bases of 100% identity to a third portion of the human B2M gene, and (v) an RRS (RNA binding protein recognition sequence) that binds a heterologous gene modifying protein.
48. The heterologous gene modifying system of embodiment 47, wherein the heterologous object sequence comprises the nucleotides of an RT template sequence from Table 1B, 2B, or 3B, a sequence having at least 5, 6, 7, 8, 9, 10, 11, 12, 13, 14, or 15 nucleotides starting with the 3′ end of the RT template sequence of Table 1B, 2B, or 3B, or a sequence having 1, 2, or 3 substitutions thereto.
49. The heterologous gene modifying system of embodiment 47, wherein the heterologous object sequence comprises the nucleotides of the RT template sequence of Table 1B, 2B, or 3B that corresponds to the gRNA spacer sequence, or a sequence having 1, 2, or 3 substitutions thereto.
50. The heterologous gene modifying system of any one of embodiments 47-49, wherein the PBS sequence has a sequence comprising the nucleotides of the PBS sequence from the same row of Table 1B, 2B, or 3B as the RT template sequence, a sequence having at least 5, 6, 7, 8, 9, 10, 11, 12, 13, 14, 15, 16, or 17 nucleotides starting with the 5′ end of a PBS sequence from Table 1B, 2B, or 3B, or a sequence having 1, 2, or 3 substitutions thereto.
51. The heterologous gene modifying system of one of embodiments 47-49, wherein the PBS sequence has a sequence comprising the nucleotides of a PBS sequence of Table 1B, 2B, or 3B that corresponds to the RT template sequence, or a sequence having 1, 2, or 3 substitutions thereto, the gRNA spacer sequence, or both.
52. The heterologous gene modifying system of any one of embodiments 47-51, wherein the gRNA scaffold comprises a sequence of a gRNA scaffold of any of Tables 1B, 2B, 3B, or 12, or a sequence having at least 70%, 75%, 80%, 85%, 90%, 95%, 96%, 97%, 98%, or 99% identity thereto.
53. The heterologous gene modifying system of any one of embodiments 17-51, wherein the gRNA scaffold comprises a sequence of a gRNA scaffold of any of Tables 1B, 2B, 3B, or 12 that corresponds to the RT template sequence, the gRNA spacer sequence, or both, or a sequence having at least 70%, 75%, 80%, 85%, 90%, 95%, 96%, 97%, 98%, or 99% identity thereto.
54. A heterologous gene modifying system for modifying DNA, comprising:
   (a) a first RNA comprising, from 5′ to 3, (i) a guide RNA sequence that is complementary to a first portion of the human B2M gene, and (ii) a sequence (e.g., a scaffold region) that binds a heterologous gene modifying polypeptide (e.g., binds the Cas domain of the heterologous gene modifying polypeptide), and
   (b) a second RNA comprising (iii) a heterologous object sequence comprising a nucleotide substitution to introduce a mutation into a second portion of the human B2M gene, wherein the heterologous object sequence comprises the nucleotides of an RT template sequence of Table 1B, 2B, or 3B, a sequence having at least 4, 5, 6, 7, 8, 9, 10, 11, 12, 13, 14, or 15 nucleotides starting with the 3′ end of the RT template sequence of Table 1B, 2B, or 3B, or a sequence having 1, 2, or 3 substitutions thereto, and (iv) a primer region comprising at least 5, 6, 7, or 8 bases of 100% homology to a third portion of the human B2M gene, and (v) an RRS (RNA binding protein recognition sequence) that binds a heterologous gene modifying protein.
55. The heterologous gene modifying system of embodiment 54, wherein the gRNA spacer comprises the nucleotides of a gRNA spacer sequence of Table 1B, 2B, or 3B, or a sequence having 1, 2, or 3 substitutions thereto.
56. The heterologous gene modifying system of embodiment 54, wherein the heterologous object sequence comprises the nucleotides of the gRNA spacer sequence of Table 1B, 2B, or 3B that corresponds to the RT template sequence, a sequence having at least 17, 18, 19, or 20 consecutive nucleotides starting with the 3′ end of the gRNA spacer of Table 1B, 2B, or 3B, or a sequence having 1, 2, or 3 substitutions thereto.
57. The heterologous gene modifying system of any one of embodiments 54-56, wherein the PBS sequence has a sequence comprising the nucleotides of the PBS sequence from the same row of Table 1B, 2B, or 3B as the RT template sequence, a sequence having at least 5, 6, 7, 8, 9, 10, 11, 12, 13, 14, 15, 16, or 17 nucleotides starting with the 5′ end of a PBS sequence from Table 1B, 2B, or 3B, or a sequence having 1, 2, or 3 substitutions thereto.
58. The heterologous gene modifying system of any one of embodiments 54-56, wherein the PBS sequence has a sequence comprising the nucleotides of a PBS sequence of Table 1B, 2B, or 3B that corresponds to the RT template sequence, the gRNA spacer sequence, or both, or a sequence having 1, 2, or 3 substitutions thereto.
59. The heterologous gene modifying system of any one of embodiments 54-58, wherein the gRNA scaffold comprises a sequence of a gRNA scaffold of any of Tables 1B, 2B, 3B, or 12, or a sequence having at least 70%, 75%, 80%, 85%, 90%, 95%, 96%, 97%, 98%, or 99% identity thereto.
60. The heterologous gene modifying system of any one of embodiments 54-58, wherein the gRNA scaffold comprises a sequence of a gRNA scaffold of any of Tables 1B, 2B, 3B, or 12 that corresponds to the RT template sequence, the gRNA spacer sequence, or both, or a sequence having at least 70%, 75%, 80%, 85%, 90%, 95%, 96%, 97%, 98%, or 99% identity thereto.
61. A gRNA comprising (i) a gRNA spacer sequence that is complementary to a first portion of the human TRAC gene, wherein the gRNA spacer has a sequence comprising the nucleotides of a gRNA spacer sequence of Table 1A, 2A, or 3A, a sequence having at least 17, 18, 19, or 20 consecutive nucleotides starting with the 3′ end of the gRNA spacer of Table 1A, 2A, or 3A, or a sequence having 1, 2, or 3 substitutions thereto; and (ii) a gRNA scaffold.
62. The gRNA of embodiment 61, wherein the gRNA scaffold comprises a sequence of a gRNA scaffold of any of Tables 1A, 2A, 3A, 12, 14B, or 14C, or a sequence having at least 70%, 75%, 80%, 85%, 90%, 95%, 96%, 97%, 98%, or 99% identity thereto.
63. The gRNA of embodiment 61, wherein the gRNA scaffold comprises a sequence of a gRNA scaffold of any of Tables 1A, 2A, 3A, 12, 14B, or 14C that corresponds to the gRNA spacer sequence, or a sequence having at least 70%, 75%, 80%, 85%, 90%, 95%, 96%, 97%, 98%, or 99% identity thereto.
64. A gRNA comprising (i) a gRNA spacer sequence that is complementary to a first portion of the human B2M gene, wherein the gRNA spacer has a sequence comprising the nucleotides of a gRNA spacer sequence of Table 1B, 2B, or 3B, a sequence having at least 17, 18, 19, or 20 consecutive nucleotides starting with the 3′ end of the gRNA spacer of Table 1B, 2B, or 3B, or a sequence having 1, 2, or 3 substitutions thereto; and (ii) a gRNA scaffold.
65. The gRNA of embodiment 64, wherein the gRNA scaffold comprises a sequence of a gRNA scaffold of any of Tables 1B, 2B, 3B, or 12, or a sequence having at least 70%, 75%, 80%, 85%, 90%, 95%, 96%, 97%, 98%, or 99% identity thereto.
66. The gRNA of embodiment 64, wherein the gRNA scaffold comprises a sequence of a gRNA scaffold of any of Tables 1B, 2B, 3B, or 12 that corresponds to the gRNA spacer sequence, or a sequence having at least 70%, 75%, 80%, 85%, 90%, 95%, 96%, 97%, 98%, or 99% identity thereto.
67. A template RNA comprising: (iii) a heterologous object sequence comprising a mutation region to introduce a mutation into a second portion of the human TRAC gene, wherein the heterologous object sequence comprises the nucleotides of an RT template sequence of Table 1A, 2A, or 3A, a sequence having at least 5, 6, 7, 8, 9, 10, 11, 12, 13, 14, or 15 nucleotides starting with the 3′ end of the RT template sequence of Table 1A, 2A, or 3A, or a sequence having 1, 2, or 3 substitutions thereto, and (iv) a PBS sequence comprising at least 5, 6, 7, or 8 bases of 100% homology to a third portion of the human TRAC gene.
68. The template RNA according to embodiment 67, wherein the PBS sequence has a sequence comprising the nucleotides of the PBS sequence from the same row of Table 1A, 2A, or 3A as the RT template sequence, a sequence having at least 5, 6, 7, 8, 9, 10, 11, 12, 13, 14, 15, 16, or 17 nucleotides starting with the 5′ end of a PBS sequence from Table 1A, 2A, or 3A, or a sequence having 1, 2, or 3 substitutions thereto.
69. The template RNA according to embodiment 67, wherein the PBS sequence has a sequence comprising the nucleotides of a PBS sequence of Table 1A, 2A, or 3A that corresponds to the RT template sequence, or a sequence having 1, 2, or 3 substitutions thereto.
70. The template RNA according to any one of embodiments 1-14, 30-32, or 67-69, the heterologous gene modifying system of any one of embodiments 33-46, or the gRNA of any one of embodiments 61-63, wherein the mutation introduced by the system is an insertion or deletion mutation of the TRAC gene.
71. A template RNA comprising: (iii) a heterologous object sequence comprising a mutation region to introduce a mutation into a second portion of the human B2M gene, wherein the heterologous object sequence comprises the nucleotides of an RT template sequence of Table 1B, 2B, or 3B, a sequence having at least 4, 5, 6, 7, 8, 9, 10, 11, 12, 13, 14, or 15 nucleotides starting with the 3′ end of the RT template sequence of Table 1B, 2B, or 3B, or a sequence having 1, 2, or 3 substitutions thereto, and (iv) a PBS sequence comprising at least 5, 6, 7, or 8 bases of 100% homology to a third portion of the human B2M gene.
72. The template RNA according to embodiment 71, wherein the PBS sequence has a sequence comprising the nucleotides of the PBS sequence from the same row of Table 1B, 2B, or 3B as the RT template sequence, a sequence having at least 5, 6, 7, 8, 9, 10, 11, 12, 13, 14, 15, 16, or 17 nucleotides starting with the 5′ end of a PBS sequence from Table 1B, 2B, or 3B, or a sequence having 1, 2, or 3 substitutions thereto.
73. The template RNA according to embodiment 71, wherein the PBS sequence has a sequence comprising the nucleotides of a PBS sequence of Table 1B, 2B, or 3B that corresponds to the RT template sequence, or a sequence having 1, 2, or 3 substitutions thereto.
74. The template RNA according to any one of embodiments 15-32 or 71-73, the heterologous gene modifying system of any one of embodiments 47-60, or the gRNA of any one of embodiments 64-66, wherein the mutation introduced by the system is an insertion or deletion mutation of the B2M gene.
75. The template RNA according to any one of embodiments 1-32 or 67-74 or the heterologous gene modifying system of any one of embodiments 33-60 or 74, wherein the pre-edit sequence comprises between about 1 nucleotide to about 35 nucleotides (e.g., comprises about 1-5, 5-10, 10-15, 15-20, 20-25, 25-30, or 30-35 nucleotides) in length.
76. The template RNA according to any one of embodiments 1-32 or 67-75 or the heterologous gene modifying system of any one of embodiments 33-60, 74, or 75, wherein the mutation region comprises at least 5 nucleotides (e.g., at least 5 nucleotides, at least 7 nucleotides, or more).
77. The template RNA according to any one of embodiments 1-32 or 67-75 or the heterologous gene modifying system of any one of embodiments 33-60, 74, or 75, wherein the mutation region is at least 7 nucleotides in length.
78. The template RNA of any one of embodiments 1-32 or 67-75, or the heterologous gene modifying system of any one of embodiments 33-60, 74, or 75, wherein the mutation region is at between about 2 to 5 nucleotides in length.
79. The template RNA according to any one of embodiments 1-32, 67-75, 77, or 78 or the heterologous gene modifying system of any one of embodiments 33-60, 74, 75, 77, or 78, wherein the mutation region is up to 32 (e.g., up to 5, 7, 10, 15, 20, 25, 30, or 32) nucleotides in length and comprises at least 5 sequence differences relative to a second portion of the human TRAC or B2Mgene.
80. The template RNA according to any one of embodiments 1-32, 67-75, or 77-79 or the heterologous gene modifying system of any one of embodiments 33-60, 74, 75, or 77-79, wherein the mutation region comprises a first region (e.g., a first nucleotide) designed to install a mutation in the TRAC gene or B2M gene and a second region (e.g., a second nucleotide) designed to inactivate a PAM sequence.
81. The template RNA according to any one of embodiments 1-32, 67-75, or 77-79 or the heterologous gene modifying system of any one of embodiments 33-60, 74, 75, or 77-79, wherein the mutation region comprises less than 80%, 70%, 60%, 50%, 40%, or 30% identity to corresponding portion of the human TRAC gene or B2M gene.
82. A heterologous gene modifying system comprising:
   a template RNA of any of embodiments 1-32, 67-75, or 77-79, or a system of any of embodiments 33-60, 74, 75, or 77-79, and
   a heterologous gene modifying polypeptide, or a nucleic acid (e.g., RNA) encoding the heterologous gene modifying polypeptide.
83. A heterologous gene modifying system comprising:
   a template RNA for introducing a mutation into a human TRAC gene, e.g., a template RNA of any of embodiments 1-14, 30-32, 67-75, or 77-79, or a system of any of embodiments 33-46 or 74, 75, or 77-79,
   a template RNA for introducing a mutation into a human B2M gene, e.g., a template RNA of any of embodiments 15-32, 71-73, or 77-79, or a system of any of embodiments 47-60, 74, 75, or 77-79,
   a heterologous gene modifying polypeptide, or a nucleic acid (e.g., RNA) encoding the heterologous gene modifying polypeptide.
84. The heterologous gene modifying system of embodiment 82 or 83, wherein the heterologous gene modifying polypeptide comprises:
   a reverse transcriptase (RT) domain (e.g., an RT domain from a retrovirus, or a polypeptide domain having at least 75%, 80%, 85%, 90%, 95%, 96%, 97%, 98%, or 99% amino acids sequence identity thereto); and
   a Cas domain that binds to the target DNA molecule and is heterologous to the RT domain (e.g., a Cas9 domain); and
   optionally, a linker disposed between the RT domain and the Cas domain.
85. The heterologous gene modifying system of embodiment 84, wherein the RT domain comprises:
   (a) an RT domain of Table 6; or
   (b) an RT domain from a murine leukemia virus (MMLV), a porcine endogenous retrovirus (PERV); Avian reticuloendotheliosis virus (AVIRE), a feline leukemia virus (FLV), simian foamy virus (SFV) (e.g., SFV3L), bovine leukemia virus (BLV), Mason-Pfizer monkey virus (MPMV), human foamy virus (HFV), or bovine foamy/syncytial virus (BFV/BSV).
86. The heterologous gene modifying system of embodiment 84 or 85, wherein the Cas domain comprises a Cas domain of Table 7 or Table 8.
87. The heterologous gene modifying system of any one of embodiments 84-86, wherein the Cas domain:
   (a) is a Cas9 domain;
   (b) is a SpCas9 domain, a BlatCas9 domain, a Nme2Cas9 domain, a PnpCas9 domain, a SauCas9 domain, a SauCas9-KKH domain, a SauriCas9 domain, a SauriCas9-KKH domain, a ScaCas9-Sc++ domain, a SpyCas9 domain, a SpyCas9-NG domain, a SpyCas9-SpRY domain, or a St1Cas9 domain; and/or
   (c) is a Cas9 domain comprising an N670A mutation, an N611A mutation, an N605A mutation, an N580A mutation, an N588A mutation, an N872A mutation, an N863 mutation, an N622A mutation, or an H840A mutation.
88. The heterologous gene modifying system of embodiment 87, wherein the Cas9 domain binds a PAM sequence listed in Table 7 or Table 12.
89. The heterologous gene modifying system of embodiment 88, wherein a second portion of the human TRAC or B2M gene overlaps with a PAM recognized by the Cas domain, e.g., wherein the second portion of the human TRAC gene or B2M gene is within the PAM or wherein the PAM is within the second portion of the human TRAC gene or B2M gene).
90. The heterologous gene modifying system of any one of embodiments 83-89, wherein:
   (a) the template RNA comprises a sequence of a template RNA sequence of Table 1A, 1B, 2A, 2B, 3A, or 3B;
   (b) the Cas domain comprises a Cas domain of Table 7 or Table 8;
   (c) the linker comprises a linker sequence of Table 10 (e.g., of any of SEQ ID NOs: 5217, 5106, 5190, and 5218); and
   (d) the heterologous gene modifying polypeptide comprises one or two NLS sequences from Table 11 (e.g., of any of SEQ ID NOs: 5245, 5290, 5323, 5330, 5349, 5350, 5351, and 4001).
91. The heterologous gene modifying system of any one of embodiments 33-46 or 74, 75, or 77-89, wherein the gRNA spacer targets a region within exon 1 of the TRAC gene.
92. The heterologous gene modifying system of any one of embodiments 33-46 or 74, 75, or 77-91, wherein the gRNA spacer comprises a sequence of gagaaucaaaaucggugaau (SEQ ID NO: 17513).
93. The heterologous gene modifying system of any one of embodiments 33-46 or 74, 75, or 77-91, wherein the gRNA spacer comprises a sequence of gcugguacacggcaggguca (SEQ ID NO: 17514).
94. The heterologous gene modifying system of any one of embodiments 47-60, 74, 75, or 77-90, wherein the gRNA spacer targets a region within exon 1 of the B2M gene.
95. The heterologous gene modifying system of any one of embodiments 47-60, 74, 75, or 77-90 or 94, wherein the gRNA spacer comprises a sequence of acUcacgcUggaUagccUcc (SEQ ID NO: 18195).
96. The heterologous gene modifying system of any one of embodiments 47-60, 74, 75, or 77-90 or 94, wherein the gRNA spacer comprises a sequence of gagUagcgcgagcacagcUa (SEQ ID NO: 18196).
97. The heterologous gene modifying system of any of embodiments 82-96, which produces a first nick in a first strand of the human TRAC gene or the B2M gene.
98. The heterologous gene modifying system of embodiment 97, which further comprises a second strand-targeting gRNA spacer that directs a second nick to the second strand of the human TRAC gene or the B2M gene.
99. The template RNA or heterologous gene modifying system, of any one of the preceding embodiments, wherein the heterologous object sequence comprises between about 8-30, 9-25, 10-20, 11-16, or 12-15 (e.g., about 11-16) nucleotides.
100. The template RNA or heterologous gene modifying system, of any one of the preceding embodiments, wherein the mutation region comprises at least 4 or at least 5 nucleotide positions of sequence differences relative to the corresponding portion of the human TRAC gene or the B2M gene.
101. The template RNA or heterologous gene modifying system, of any one of the preceding embodiments, wherein the mutation region comprises between about 2 to 5 nucleotide positions of sequence differences relative to the corresponding portion of the human TRAC gene or the B2M gene.
102. The template RNA or heterologous gene modifying system of any one of the preceding embodiments, wherein the mutation region comprises at least 7 nucleotide positions of sequence difference relative to the corresponding portion of the human TRAC gene or the B2M gene.
103. The template RNA or heterologous gene modifying system, of any one of the preceding embodiments, wherein the post-edit homology region and/or pre-edit homology region comprises 100% identity to the TRAC gene or the B2M gene.
104. The template RNA or heterologous gene modifying system of any one of the preceding embodiments, wherein the PBS sequence comprises about 5-20, 8-16, 8-14, 8-13, 9-13, 9-12, or 10-12 (e.g., about 5-17) nucleotides.
105. The template RNA or heterologous gene modifying system of any one of the preceding embodiments, wherein the PBS sequence binds within 1, 2, 3, 4, 5, 6, 7, 8, 9, or 10 nucleotides of a nick site in the TRAC gene or the B2M gene.
106. The heterologous gene modifying system of any one of the preceding embodiments, wherein the domains of the heterologous gene modifying polypeptide are joined by a peptide linker.
107. The heterologous gene modifying system of embodiment 106, wherein the linker comprises a sequence of a linker of Table 10 (e.g., of any of SEQ ID NOs: 5217, 5106, 5190, and 5218).
108. The heterologous gene modifying system of any one of the preceding embodiments, wherein the heterologous gene modifying polypeptide further comprise one or more nuclear localization sequences (NLS).
109. The heterologous gene modifying system of embodiment 108, wherein the heterologous gene modifying polypeptide comprises a first NLS and a second NLS.
110. The heterologous gene modifying system of embodiment 108 or 109, wherein the NLS comprises a sequence of a NLS of Table 11 (e.g., of any of SEQ ID NOs: 5245, 5290, 5323, 5330, 5349, 5350, 5351, and 4001).
111. A template RNA comprising a sequence of a template RNA of Table 1A, 1B, 2A, 2B, 3A, or 3B, or a sequence having at least 70%, 75%, 80%, 85%, 90%, 95%, 96%, 97%, 98%, or 99% identity thereto.
112. A template RNA comprising a sequence of a template RNA of Table 1A, 1B, 2A, 2B, 3A, or 3B.
113. A DNA encoding the template RNA of any one of embodiments 1-32 or 67-79, 99-105, 111, or 112, or the gRNA of any one of embodiments 61-66.
114. A pharmaceutical composition, comprising the system of any one of embodiments 33-60, 74, 75, or 77-110, or one or more nucleic acids encoding the same, and a pharmaceutically acceptable excipient or carrier,
115. The pharmaceutical composition of embodiment 114, wherein the pharmaceutically acceptable excipient or carrier is selected from the group consisting of a plasmid vector, a viral vector, a vesicle, and a lipid nanoparticle.
116. The pharmaceutical composition of embodiment 115, wherein the viral vector is an adeno-associated virus.
117. A host cell (e.g., a mammalian cell, e.g., a human cell) comprising the template RNA or heterologous gene modifying system of any one of the preceding embodiments.
118. A method of making the template RNA of any one of embodiments 1-32 or 67-79, 99-105, 111, or 112, the method comprising synthesizing the template RNA by in vitro transcription (e.g., solid state synthesis) or by introducing a DNA encoding the template RNA into a host cell under conditions that allow for production of the template RNA.
119. A method for modifying a target site in the human TRAC gene and/or the B2M gene in a cell, the method comprising contacting the cell with the heterologous gene modifying system of any one of embodiments 33-60, 74, 75, or 77-110, or DNA encoding the same, thereby modifying the target site in the human TRAC gene and/or the B2M gene in a cell.
120. A method for modifying a target site in the human TRAC gene and/or the B2M gene in a cell, the method comprising contacting the cell with: (i) the template RNA of any one of embodiments 1-32 or 67-79, 99-105, 111, or 112, or DNA encoding the same; and (ii) a heterologous gene modifying polypeptide or a nucleic acid encoding a heterologous gene modifying polypeptide, thereby modifying the target site in the human TRAC gene and/or the B2M gene in a cell.
121. The heterologous gene modifying system or method of any one of the preceeding embodiments, wherein insertion of the heterologous gene modifying system into a target cell results in a mutation in that causes disruption of the TRAC gene and/or the B2M gene.
122. The heterologous gene modifying system of any of embodiments 121, wherein installation of the mutation results in a loss of TRAC or B2M expression and/or the B2M gene.
123. The heterologous gene modifying system or method of any one of the preceeding embodiments, wherein insertion of the system into a target cell results in a mutation in that disrupts splicing of the TRAC gene and/or B2M gene.
124. The heterologous gene modifying system or method of embodiment 122 or 123, wherein mutation generates a stop codon (e.g., a premature stop codon) in the TRAC gene and/or B2M gene.
125. The heterologous gene modifying system or method of any of the preceding embodiments wherein the mutation comprises insertion of between about 4 to 30 nucleotides in the TRAC gene and/or B2M gene, resulting in a frameshift in the TRAC gene and/or B2M gene.
126. The heterologous gene modifying system or method of any of the preceding embodiments wherein the mutation results in a deletion of a region of the TRAC gene and/or B2M gene.
127. The heterologous gene modifying system of embodiment 126, wherein the mutation comprises a deletion of between about 4 and 30 nucleotides in the TRAC gene and/or B2M gene.
128. The heterologous gene modifying system of any of any of the preceding embodiments, wherein the mutation is introduced at (i) a protospacer nick site or (ii) the PAM location associated with the protospacer.
129. The heterologous gene modifying system or method of any of the preceding embodiments, wherein installation of the mutation occurs in at least 15% (e.g., 15%, 20%, 25%, 30%, 40%, 50%, 60%, 70%, 75%, or more) of target nucleic acids.
130. The heterologous gene modifying system or method of any of the preceding embodiments, wherein installation of the mutation occurs in at least 15% (e.g., 15%, 20%, 25%, 30%, 40%, 50%, 60%, 70%, 75%, or more) of target cells.
131. A system for modifying DNA comprising:
   (a) a first gene modifying system comprising: (i) a retrotransposon gene modifying polypeptide or a nucleic acid (e.g., DNA or mRNA) encoding the retrotransposon gene modifying polypeptide, and (ii) a first template RNA (or DNA encoding the template RNA) comprising (1) a sequence that binds the retrotransposon gene modifying polypeptide and (2) a first heterologous object sequence; and
   (b) a second system comprising: (i) a heterologous gene modifying polypeptide or a nucleic acid (e.g., DNA or mRNA) encoding the heterologous gene modifying polypeptide, and
   (ii) a second template RNA (or DNA encoding the template RNA) comprising (1) a gRNA spacer, (2) a gRNA scaffold, (3) a heterologous object sequence, and (4) a primer binding site (PBS) sequence.
132. The system of embodiment 131, wherein the second system comprises the heterologous gene modifying system of any of embodiments 33-60, 74, 75, or 77-110.
133. A system for modifying DNA comprising:
   (a) a first gene modifying system comprising: (i) a retrotransposon gene modifying polypeptide or a nucleic acid (e.g., DNA or mRNA) encoding the retrotransposon gene modifying polypeptide, and (ii) a first template RNA (or DNA encoding the template RNA) comprising (1) a sequence that binds the retrotransposon gene modifying polypeptide and (2) a first heterologous object sequence; and
   (b) a second system comprising: (i) a heterologous gene modifying polypeptide or a nucleic acid (e.g., DNA or mRNA) encoding the heterologous gene modifying polypeptide, (ii) a second template RNA (or DNA encoding the template RNA) comprising (1) a gRNA spacer, (2) a gRNA scaffold, (3) a heterologous object sequence, and (4) a primer binding site (PBS) sequence; and (iii) a third template RNA (or DNA encoding the template RNA) comprising (1) a gRNA spacer, (2) a gRNA scaffold, (3) a heterologous object sequence, and (4) a primer binding site (PBS) sequence.
134. The system of embodiment 133, wherein the second system comprises the heterologous gene modifying system of any of embodiments 33-60, 74, 75, or 77-110.
135. A system for modifying DNA comprising:
   (a) a first gene modifying system comprising: (i) a retrotransposon gene modifying polypeptide or a nucleic acid (e.g., DNA or mRNA) encoding the retrotransposon gene modifying polypeptide, and (ii) a first template RNA (or DNA encoding the template RNA) comprising (1) a sequence that binds the retrotransposon gene modifying polypeptide and (2) a first heterologous object sequence (e.g., encoding a CAR); and
   (b) a second system comprising: (i) a heterologous gene modifying polypeptide or a nucleic acid (e.g., DNA or mRNA) encoding the heterologous gene modifying polypeptide, (ii) a second template RNA (or DNA encoding the template RNA) of any one of embodiments 1-14, 30-32, 67-75, 77-79, 99-105, 111, or 112; and (iii) a third template RNA (or DNA encoding the template RNA) of any one of embodiments 15-32, 71-73, 77-79, 99-105, 111, or 112.
136. The system of any of embodiments 131-135, wherein the retrotransposon gene modifying polypeptide comprises an amino acid sequence of Table R1 or a sequence having no more than 10, 20, 30, 40, 50, 60, 70, 80, 90, or 100 amino acid differences thereto or a sequence having at least 80%, 85%, 90%, 95%, 97%, 98%, or 99% identity thereto, or a nucleic acid (e.g., DNA or mRNA) encoding the retrotransposon gene modifying polypeptide.
137. The system of any one of embodiments 131-136, wherein the retrotransposon gene modifying polypeptide comprises an amino acid sequence listed in Example 11 or Example 12 or a sequence having no more than 10, 20, 30, 40, 50, 60, 70, 80, 90, or 100 amino acid differences thereto or a sequence having at least 80%, 85%, 90%, 95%, 97%, 98%, or 99% identity thereto, or a nucleic acid (e.g., DNA or mRNA) encoding the retrotransposon gene modifying polypeptide.
138. The system of any of embodiments 131-137, wherein the sequence that binds the polypeptide comprises a 5′UTRᵣₑₜᵣₒ or a 3′ UTRᵣₑₜᵣₒ.
139. The system of embodiment 138, wherein the first template RNA comprises both of a 5′UTRᵣₑₜᵣₒ and a 3′ UTRᵣₑₜᵣₒ.
140. The system of embodiment 139, wherein the 5′UTRᵣₑₜᵣₒ and the 3′ UTRᵣₑₜᵣₒ comprise 5′ and 3′ sequences of Table R1 or Example 11 or Example 12.
141. The system of any of embodiments 131-140, wherein the first heterologous object sequence encodes a chimeric antigen receptor (CAR), wherein the CAR comprises an antigen-binding domain, a transmembrane domain, a first intracellular signaling domain, and a second intracellular signaling domain.
142. The gene modifying system of any of embodiments 131-141, wherein the heterologous gene modifying polypeptide comprises:
   a reverse transcriptase (RT) domain (e.g., an RT domain from a retrovirus, or a polypeptide domain having at least 75%, 80%, 85%, 90%, 95%, 96%, 97%, 98%, or 99% amino acids sequence identity thereto); and
   a Cas domain that binds to the target DNA molecule and is heterologous to the RT domain (e.g., a Cas9 domain); and optionally, a linker disposed between the RT domain and the Cas domain.
143. The system of embodiment 142, wherein the RT domain comprises an amino acid sequence of Table 6, or a sequence having at least 70%, 80%, 85%, 90%, 95%, 97%, 98%, or 99% identity thereto.
144. The gene modifying system of embodiment 142 or 143, wherein the Cas domain comprises a Cas domain of Table 7 or Table 8A, or a sequence having at least 70%, 80%, 85%, 90%, 95%, 97%, 98%, or 99% identity thereto.
145. A method of modifying the genome of a mammalian cell, comprising contacting a population of cells with a system of any one of embodiments 131-144, thereby modifying the genome of a cell of the population.
146. The method of embodiment 145, wherein the first gene modifying system produces a first sequence alteration (e.g., an insertion) and the second system produces a second sequence alteration in the genome of the mammalian cell.
147. The method of embodiment 146, wherein at least 5%, 10%, or 20% of cells in the population comprise the first sequence alteration.
148. The method of any embodiment 146 or 147, wherein at least 10%, 20%, 30%, 40%, 50%, or 60% of cells in the population comprise the second sequence alteration.
149. The method of any one of embodiments 146-148, wherein at least 20%, 40%, 60%, 80% of cells that comprise the first sequence alteration also comprise the second sequence alteration.
150. A method for modifying the genome of a mammalian cell, the method comprising contacting a population of cells with a heterologous gene modifying system comprising:
   (i) a heterologous gene modifying polypeptide or a nucleic acid (e.g., DNA or mRNA) encoding the heterologous gene modifying polypeptide,
   (ii) a first template RNA (or DNA encoding the template RNA) comprising (1) a gRNA spacer, (2) a gRNA scaffold, (3) a heterologous object sequence, and (4) a primer binding site (PBS) sequence, wherein the first template RNA is configured to produce a first mutation; and
   (iii) a second template RNA (or DNA encoding the template RNA) comprising (1) a gRNA spacer, (2) a gRNA scaffold, (3) a heterologous object sequence, and (4) a primer binding site (PBS) sequence, wherein the second template RNA is configured to produce a second mutation,
   wherein contacting the population of cells with the system results in installation of the first and second mutations (e.g., insertions) in at least 40%, 50%, 60%, 70%, 75% of said cells.
151. The method of embodiment 150, wherein the first template RNA comprises a gRNA spacer that is complementary to portion of the human TRAC gene (e.g., comprises a gRNA spacer sequence of Table 1A, 2A, or 3A).
152. The method of embodiment 150 or 151, wherein the second template RNA comprises a gRNA spacer that is complementary to portion of the human B2M gene (e.g., comprises a gRNA spacer sequence of Table 1B, 2B, or 3B).
153. The method of any one of embodiments 150-152, wherein the modifying does not result in a translocation event.
154. The method of any one of embodiments 119-130 or 145-153, wherein the modifying does not result in a translocation event.
155. The method of any of the preceding embodiments, wherein the cell is a mammalian cell, such as a human cell.
156. The method of embodiment 155, wherein the mammalian cell is an immune effector cell.
157. The method of embodiment 155 or 156, wherein the mammalian cell is a T cell, e.g., a primary T cell.
158. The method of any one of the preceding embodiments, wherein the subject is a human.
159. The method of any of the preceding embodiments, wherein the contacting occurs ex vivo, e.g., wherein the cell's or subject's DNA is modified ex vivo.
160. The method of any of the preceding embodiments, wherein the contacting occurs in vivo, e.g., wherein the cell's or subject's DNA is modified in vivo.
161. The method of any of the preceding embodiments, wherein contacting the cell or the subject with the system comprises contacting the cell or a cell within the subject with a nucleic acid (e.g., DNA or RNA) encoding the heterologous gene modifying polypeptide under conditions that allow for production of the heterologous gene modifying polypeptide.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts a heterologous gene modifying system as described herein. The left hand diagram shows the heterologous gene modifying polypeptide, which comprises a Cas nickase domain (e.g., spCas9 N863A) and a reverse transcriptase domain (RT domain) which are linked by a linker. The right hand diagram shows the template RNA which comprises, from 5′ to 3′, a gRNA spacer, a gRNA scaffold, a heterologous object sequence, and a primer binding site sequence (PBS sequence). The heterologous object sequence can comprise a mutation region that comprises one or more sequence differences relative to the target site. The heterologous object sequence can also comprise a pre-edit homology region and a post-edit homology region, which flank the mutation region. Without wishing to be bound by theory, it is thought that the gRNA spacer of the template RNA binds to the second strand of a target site in the genome, and the gRNA scaffold of the template RNA binds to the heterologous gene modifying polypeptide, e.g., localizing the heterologous gene modifying polypeptide to the target site in the genome. It is thought that the Cas domain of the heterologous gene modifying polypeptide nicks the target site (e.g., the first strand of the target site), e.g., allowing the PBS sequence to bind to a sequence adjacent to the site to be altered on the first strand of the target site. It is thought that the RT domain of the heterologous gene modifying polypeptide uses the first strand of the target site that is bound to the complementary sequence comprising the PBS sequence of the template RNA as a primer and the heterologous object sequence of the template RNA as a template to, e.g., polymerize a sequence complementary to the heterologous object sequence. Without wishing to be bound by theory, it is thought that reverse transcription can then proceed through the pre-edit homology region, then through the mutation region, and then through the post-edit homology region, thereby producing a DNA strand comprising a mutation specified by the heterologous object sequence.

FIG. 2 is a graph showing editing % as assessed by Amp-SEQ for primary human T cells nucleofected with select exemplary heterologous gene modifying system containing TRAC4 template RNAs designed to produce 5 nucleotide TAGTG insertions at the nick site to disrupt (e.g., knock out) TRAC expression.

FIG. 3 is a graph showing editing % as assessed by Amp-SEQ for primary human T cells nucleofected with select exemplary heterologous gene modifying system containing TRAC4 template RNAs designed to produce 7 nucleotide deletions at the nick site to disrupt (e.g., knock out) TRAC expression.

FIG. 4 is a graph showing editing % as assessed by flow cytometry for primary human T cells nucleofected with select exemplary heterologous gene modifying system containing TRAC4 insertion and deletion template RNAs designed to produce 5 nucleotide TAGTG insertions, 7 nucleotide TAGTTGA insertions, 5 nucleotide deletions, or 7 nucleotide deletions at either the nick site or the PAM site, respectively, to disrupt (e.g., knock out) TRAC expression.

FIG. 5 is a graph showing editing % as assessed by Amp-SEQ for primary human T cells nucleofected with select exemplary heterologous gene modifying system containing TRAC5 template RNAs designed to produce 5 nucleotide TAGTG insertions at the nick site to disrupt (e.g., knock out) TRAC expression.

FIG. 6 is a graph showing editing % as assessed by Amp-SEQ for primary human T cells nucleofected with select exemplary heterologous gene modifying system containing TRAC5 template RNAs designed to produce 7 nucleotide deletions at the nick site to disrupt (e.g., knock out) TRAC expression.

FIG. 7 is a graph showing knock out phenotype % as assessed by flow cytometry for primary human T cells nucleofected with select exemplary heterologous gene modifying system containing TRAC5 insertion and deletion template RNAs designed to produce 5 nucleotide TAGTG insertions, 7 nucleotide TAGTTGA insertions, 5 nucleotide deletions, or 7 nucleotide deletions at either the nick site or the PAM site, respectively, to disrupt (e.g., knock out) TRAC expression.

FIG. 8 is a graph showing editing % as assessed by Amp-SEQ for primary human T cells nucleofected with select exemplary heterologous gene modifying system containing template RNAs designed to disrupt (e.g., knock out) TRAC expression.

FIGS. 9A-9D is a series of graphs showing % knock out as assessed by flow cytometry for primary human T cells nucleofected with varying doses of select exemplary heterologous gene modifying systems containing TRAC5 insertion template RNAs (FIG. 9A), TRAC5 deletion template RNAs (FIG. 9B), TRAC4 insertion template RNAs (FIG. 9C), or TRAC4 deletion template RNAs (FIG. 9D).

FIG. 10A is a graph showing percent editing as assessed by Amp-SEQ for primary human T cells nucleofected with select exemplary heterologous gene modifying system containing B2M1 template RNAs designed to produce 4 nucleotide TGAT (v1) or TAAC (v2) insertions at the nick site to knock out B2M expression.

FIG. 10B is a graph showing percent editing as measured by Amp-SEQ in primary human T cells nucleofected with select exemplary heterologous gene modifying system containing B2M1 template RNAs designed to produce 5 nucleotide TGATG (v1) or TAACC (v2) insertions at the nick site to knock out B2M expression.

FIGS. 10C and 10D are graphs showing knockout efficiencies as measured by flow cytometry from the same experiment and the same select template RNAs as shown in FIG. 10A and FIG. 10B, respectively.

FIG. 11A is a graph showing percent editing as assessed by Amp-SEQ for primary human T cells nucleofected with select exemplary heterologous gene modifying system containing B2M2 template RNAs designed to produce 4 nucleotide TGAT (v1) or TAAC (v2) insertions at the nick site to knock out B2M expression.

FIG. 11B is a graph showing percent editing as measured by Amp-SEQ in primary human T cells nucleofected with select exemplary heterologous gene modifying system containing B2M2 template RNAs designed to produce 5 nucleotide TGATG (v1) and TAACC (v2) insertions at the nick site to knock out B2M expression.

FIGS. 11C and 11D are graphs showing knockout efficiencies as measured by flow cytometry from the same experiment and the same select template RNAs as shown in FIG. 11A and FIG. 11B, respectively.

FIGS. 12A and 12B are graphs showing percent knockout as assessed by flow cytometry for primary human T cells nucleofected with select exemplary heterologous gene modifying system containing B2M1 or B2M2 template RNAs designed to produce 4 nucleotide insertions (TGAT (v1) or TAAC (v2)) insertions or 5 nt insertions (TGATG (v1) or TAACC (v2)) at the nick site to knock out B2M expression.

FIG. 13 is a graph showing percent knockout as assessed by flow cytometry for primary human T cells nucleofected with select exemplary heterologous gene modifying system containing B2M1 or B2M2 template RNAs designed to produce 4 nucleotide insertions (TGAT (v1) or TAAC (v2)) insertions or 5 nt insertions (TGATG (v1) or TAACC (v2)) at the nick site to knock out B2M expression.

FIG. 14 is a graph showing the levels of editing by the retrotransposon gene modifying system in the bulk T cell population when the cells were transfected with the retrotransposon gene modifying system, either alone or in combination with the heterologous gene modifying system.

FIG. 15 is a graph showing the levels of editing by the heterologous gene modifying system (containing a template RNA designed to produce an insertion in TRAC and a template RNA designed to produce an insertion in B2M) in the bulk T cell population when the cells were transfected with the heterologous gene modifying system either alone or in combination with the retrotransposon gene modifying system.

FIG. 16 is a graph showing the levels of cells edited by the heterologous gene modifying system (measured here as combined loss of expression of CD3 and B2M by flow cytometry) and also edited by the retrotransposon gene editing system (that were GFP+ or BCMA CAR+ T cells).

FIG. 17 is a graph showing a phenotypic characterization by flow cytometry of T cells treated with the heterologous gene modifying system (containing a template RNA designed to produce an insertion in TRAC and a template RNA designed to produce an insertion in B2M), both the heterologous and retrotransposon gene modifying systems, or a mock treatment lacking both gene modifying systems.

FIG. 18 is a graph showing percent cytokine expressing cells as assessed by flow cytometry of T cells edited with the heterologous gene modifying system (containing a template RNA designed to produce an insertion in TRAC and a template RNA designed to produce an insertion in B2M), edited with both the heterologous and retrotransposon gene modifying systems, or a mock treated cells edited by neither gene modifying systems.

FIG. 19 is a graph showing the percentage of edited activated T cells at the TRAC and B2M loci by a first gene modifying system comprising a WT Cas9-RT fusion polypeptide and second heterologous gene modifying system comprising an exemplary heterologous gene modifying polypeptide.

FIG. 20 is a graph showing percent translocation in T cells following gene editing at the TRAC and B2M loci by a first gene modifying system comprising a WT Cas9-RT fusion polypeptide and second heterologous gene modifying system comprising an exemplary heterologous gene modifying polypeptide.

FIG. 21 is a graph showing the percent rewriting achieved using the RNAV209-013 or RNAV214-040 heterologous gene modifying polypeptides with the indicated template RNAs.

FIG. 22 is a graph showing the amount of Fah mRNA relative to wild type when template RNAs are used with the RNAV209-013 or RNAV214-040 heterologous gene modifying polypeptides.

FIG. 23 is a graph showing the percentage of Cas9-positive hepatocytes 6 hours following dosing with LNPs containing various heterologous gene modifying polypeptides and template RNAs.

FIG. 24 is a graph showing the rewrite levels in liver samples 6 days following dosing with LNPs containing various heterologous gene modifying polypeptides and template RNAs.

FIG. 25 is a graph showing wild type Fah mRNA restoration compared to littermate heterozygous mice in liver samples following dosing with LNPs containing various heterologous gene modifying polypeptides and template RNAs.

FIG. 26 is a graph showing Fah protein distribution in liver samples following dosing with LNPs containing various heterologous gene modifying polypeptides and template RNAs.

FIG. 27 is a series of western blots showing Cas9-RT expression 6 hours after infusion of Cas9-RT mRNA+TTR guide LNP. Each lane represents an individual animal where 20 μg of tissue homogenate was added per lane. Positive control was from an in vitro cell experiment where Cas9-RT was expressed (described previously). GAPDH was used as a loading control for each sample. n=4 per group, vehicle or treated.

FIG. 28 is a graph showing gene editing of TTR locus after treatment with Cas9-RT mRNA+TTR guide LNP. Level of indels detected at the TTR locus measured by TIDE analysis of Sanger sequencing of the TTR locus where the protospacer targets.

FIG. 29 is a graph showing that TTR serum levels decrease after treatment with Cas9-RT mRNA+TTR guide LNP. Measurement of circulating TTR levels 5 days after mice were treated with LNPs encapsulating Cas9-RT+TTR guide RNA.

FIG. 30 is a graph showing Cas9-RT Expression after infusion of Cas9-RT mRNA+TTR guide LNP. Relative expression quantified by ProteinSimple Jess capillary electrophoresis Western blot. Numbers in the symbols are animal number in group. Vehicle n=2, Cas9-RT+TTR guide n=3.

FIG. 31 is a graph showing gene editing of TTR locus after infusion of Cas9-RT mRNA+TTR guide LNP. Level of indels detected at the TTR locus were measured by amplicon sequencing of the TTR locus where the protospacer targets. Each animal had 8 different biopsies taken across the liver where amplicon sequencing measured the percentage of reads showing an indel.

FIGS. 32A and 32B are pair of graphs showing the levels of editing by the first heterologous gene modifying system and the second retrotransposon gene modifying system in the bulk T cell population when the cells were co-transfected with LNPs formulated with a payload comprising the first heterologous gene modifying system and LNPs formulated with a payload comprising the second retrotransposon gene modifying system designed to integrate a GFP sequence (32A) or a CAR sequence (32B) into the genome of cells.

FIG. 33 is a graph showing the percent indels or percent rewriting in B2M in T cells following administration of LNPs to a humanized mouse model.

### DETAILED DESCRIPTION

### Definitions

The term “antigen binding domain” as used herein refers to that portion of antibody or a chimeric antigen receptor which binds an antigen. In some embodiments, an antigen binding domain binds to a cell surface antigen of a cell. In some embodiments an antigen binding domain binds an antigen characteristic of a cancer, e.g., a tumor associated antigen in a neoplastic cell. In some embodiments, an antigen binding domain binds an antigen characteristic of an infectious disease, e.g. a virus associated antigen in a virus infected cell. In some embodiments, an antigen binding domain binds an antigen characteristic of a cell targeted by a subject's immune system in an autoimmune disease, e.g., a self-antigen. In some embodiments, an antigen binding domain is or comprises an antibody or antigen-binding portion thereof. In some embodiments, an antigen binding domain is or comprises an scFv or Fab.

The term “expression cassette,” as used herein, refers to a nucleic acid construct comprising nucleic acid elements sufficient for the expression of the nucleic acid molecule of the instant invention.

A “gRNA spacer”, as used herein, refers to a portion of a nucleic acid that has complementarity to a target nucleic acid and can, together with a gRNA scaffold, target a Cas protein to the target nucleic acid.

A “gRNA scaffold”, as used herein, refers to a portion of a nucleic acid that can bind a Cas protein and can, together with a gRNA spacer, target the Cas protein to the target nucleic acid. In some embodiments, the gRNA scaffold comprises a crRNA sequence, tetraloop, and tracrRNA sequence.

A “heterologous gene modifying polypeptide”, as used herein, refers to a polypeptide comprising a retroviral reverse transcriptase, or a polypeptide comprising an amino acid sequence having at least 75%, 80%, 85%, 90%, 95%, 96%, 97%, 98%, or 99% amino acid sequence identity to a retroviral reverse transcriptase, which is capable of integrating a nucleic acid sequence (e.g., a sequence provided on a template nucleic acid) into a target DNA molecule (e.g., in a mammalian host cell, such as a genomic DNA molecule in the host cell). In some embodiments, the heterologous gene modifying polypeptide is capable of integrating the sequence substantially without relying on host machinery. In some embodiments, the heterologous gene modifying polypeptide integrates a sequence into a random position in a genome, and in some embodiments, the heterologous gene modifying polypeptide integrates a sequence into a specific target site. In some embodiments, a heterologous gene modifying polypeptide includes one or more domains that, collectively, facilitate 1) binding the template nucleic acid, 2) binding the target DNA molecule, and 3) facilitate integration of the at least a portion of the template nucleic acid into the target DNA. Heterologous gene modifying polypeptides include both naturally occurring polypeptides as well as engineered variants of the foregoing, e.g., having one or more amino acid substitutions to the naturally occurring sequence. Heterologous gene modifying polypeptides also include heterologous constructs, e.g., where one or more of the domains recited above are heterologous to each other, whether through a heterologous fusion (or other conjugate) of otherwise wild-type domains, as well as fusions of modified domains, e.g., by way of replacement or fusion of a heterologous sub-domain or other substituted domain. Exemplary heterologous gene modifying polypeptides, and systems comprising them and methods of using them, that can be used in the methods provided herein are described, e.g., in PCT/US2021/020948, which is incorporated herein by reference with respect to heterologous gene modifying polypeptides that comprise a retroviral reverse transcriptase domain. In some embodiments, a heterologous gene modifying polypeptide integrates a sequence into a gene. In some embodiments, a heterologous gene modifying polypeptide integrates a sequence into a sequence outside of a gene. A “heterologous gene modifying system,” as used herein, refers to a system comprising a heterologous gene modifying polypeptide and a template nucleic acid.

A “retrotransposon gene modifying polypeptide” as used herein to refers to a polypeptide comprising a retrotransposase reverse transcriptase domain and a retrotransposase endonuclease domain, or a polypeptide comprising an amino acid sequence having at least 75%, 80%, 85%, 90%, 95%, 96%, 97%, 98%, or 99% amino acid sequence identity to said domains, which is capable of integrating a nucleic acid sequence (e.g., a sequence provided on a template nucleic acid) into a target DNA molecule (e.g., in a mammalian host cell, such as a genomic DNA molecule in the host cell). In some embodiments, the endonuclease domain is a catalytically inactive endonuclease domain. In some embodiments, the retrotransposase reverse transcriptase domain and a retrotransposase endonuclease domain are derived from the same retrotransposase. In some embodiments, the retrotransposon gene modifying polypeptide is capable of integrating the sequence substantially without relying on host machinery. In some embodiments, the retrotransposon gene modifying polypeptide integrates a sequence into a random position in a genome, and in some embodiments, the retrotransposon gene modifying polypeptide integrates a sequence into a specific target site. In some embodiments, a retrotransposon gene modifying polypeptide includes one or more domains that, collectively, facilitate 1) binding the template nucleic acid, 2) binding the target DNA molecule, and 3) facilitate integration of the at least a portion of the template nucleic acid into the target DNA. Retrotransposon gene modifying polypeptides include both naturally occurring polypeptides as well as engineered variants of the foregoing, e.g., having one or more amino acid substitutions to the naturally occurring sequence. Retrotransposon gene modifying polypeptides also include heterologous constructs, e.g., where one or more of the domains recited above are heterologous to each other, whether through a heterologous fusion (or other conjugate) of otherwise wild-type domains, as well as fusions of modified domains, e.g., by way of replacement or fusion of a heterologous sub-domain or other substituted domain. Exemplary retrotransposon gene modifying polypeptides, and systems comprising them and methods of using them, that can be used in the methods provided herein are described, e.g., in WO/2021/178717, which is incorporated herein by reference, including Tables 10, 11, X, 3A, 3B, and Z1 therein. In some embodiments, a retrotransposon gene modifying polypeptide integrates a sequence into a gene. In some embodiments, a retrotransposon gene modifying polypeptide integrates a sequence into a sequence outside of a gene. A “retrotransposon gene modifying system,” as used herein, refers to a system comprising a retrotransposon gene modifying polypeptide and a template nucleic acid.

The term “domain” as used herein refers to a structure of a biomolecule that contributes to a specified function of the biomolecule. A domain may comprise a contiguous region (e.g., a contiguous sequence) or distinct, non-contiguous regions (e.g., non-contiguous sequences) of a biomolecule. Examples of protein domains include, but are not limited to, an endonuclease domain, a DNA binding domain, a reverse transcription domain; an example of a domain of a nucleic acid is a regulatory domain, such as a transcription factor binding domain. In some embodiments, a domain (e.g., a Cas domain) can comprise two or more smaller domains (e.g., a DNA binding domain and an endonuclease domain).

As used herein, the term “exogenous”, when used with reference to a biomolecule (such as a nucleic acid sequence or polypeptide) means that the biomolecule was introduced into a host genome, cell or organism by the hand of man. For example, a nucleic acid that is as added into an existing genome, cell, tissue or subject using recombinant DNA techniques or other methods is exogenous to the existing nucleic acid sequence, cell, tissue or subject.

As used herein, “first strand” and “second strand”, as used to describe the individual DNA strands of target DNA, distinguish the two DNA strands based upon which strand the reverse transcriptase domain initiates polymerization, e.g., based upon where target primed synthesis initiates. The first strand refers to the strand of the target DNA upon which the reverse transcriptase domain initiates polymerization, e.g., where target primed synthesis initiates. The second strand refers to the other strand of the target DNA. First and second strand designations do not describe the target site DNA strands in other respects; for example, in some embodiments the first and second strands are nicked by a polypeptide described herein, but the designations ‘first’ and ‘second’ strand have no bearing on the order in which such nicks occur.

The term “heterologous,” as used herein to describe a first element in reference to a second element means that the first element and second element do not exist in nature disposed as described. For example, a heterologous polypeptide, nucleic acid molecule, construct or sequence refers to (a) a polypeptide, nucleic acid molecule or portion of a polypeptide or nucleic acid molecule sequence that is not native to a cell in which it is expressed, (b) a polypeptide or nucleic acid molecule or portion of a polypeptide or nucleic acid molecule that has been altered or mutated relative to its native state, or (c) a polypeptide or nucleic acid molecule with an altered expression as compared to the native expression levels under similar conditions. For example, a heterologous regulatory sequence (e.g., promoter, enhancer) may be used to regulate expression of a gene or a nucleic acid molecule in a way that is different than the gene or a nucleic acid molecule is normally expressed in nature. In another example, a heterologous domain of a polypeptide or nucleic acid sequence (e.g., a DNA binding domain of a polypeptide or nucleic acid encoding a DNA binding domain of a polypeptide) may be disposed relative to other domains or may be a different sequence or from a different source, relative to other domains or portions of a polypeptide or its encoding nucleic acid. In certain embodiments, a heterologous nucleic acid molecule may exist in a native host cell genome, but may have an altered expression level or have a different sequence or both. In other embodiments, heterologous nucleic acid molecules may not be endogenous to a host cell or host genome but instead may have been introduced into a host cell by transformation (e.g., transfection, electroporation), wherein the added molecule may integrate into the host genome or can exist as extra-chromosomal genetic material either transiently (e.g., mRNA) or semi-stably for more than one generation (e.g., episomal viral vector, plasmid or other self-replicating vector).

As used herein, “insertion” of a sequence into a target site refers to the net addition of DNA sequence at the target site, e.g., where there are new nucleotides in the heterologous object sequence with no cognate positions in the unedited target site. In some embodiments, a nucleotide alignment of the PBS sequence and heterologous object sequence to the target nucleic acid sequence would result in an alignment gap in the target nucleic acid sequence.

As used herein, a “deletion” generated by a heterologous object sequence in a target site refers to the net deletion of DNA sequence at the target site, e.g., where there are nucleotides in the unedited target site with no cognate positions in the heterologous object sequence. In some embodiments, a nucleotide alignment of the PBS sequence and heterologous object sequence to the target nucleic acid sequence would result in an alignment gap in the molecule comprising the PBS sequence and heterologous object sequence.

The term “inverted terminal repeats” or “ITRs” as used herein refers to AAV viral cis-elements named so because of their symmetry. These elements promote efficient multiplication of an AAV genome. It is hypothesized that the minimal elements for ITR function are a Rep-binding site (RBS; 5′-GCGCGCTCGCTCGCTC-3′ (SEQ ID NO: 37656) for AAV2) and a terminal resolution site (TRS; 5′-AGTTGG-3′ for AAV2) plus a variable palindromic sequence allowing for hairpin formation. According to the present invention, an ITR comprises at least these three elements (RBS, TRS, and sequences allowing the formation of a hairpin). In addition, in the present invention, the term “ITR” refers to ITRs of known natural AAV serotypes (e.g. ITR of a serotype 1, 2, 3, 4, 5, 6, 7, 8, 9, 10 or 11 AAV), to chimeric ITRs formed by the fusion of ITR elements derived from different serotypes, and to functional variants thereof. “Functional variant” refers to a sequence presenting a sequence identity of at least 80%, 85%, 90%, preferably of at least 95% with a known ITR and allowing multiplication of the sequence that includes said ITR in the presence of Rep proteins.

The term “mutation region,” as used herein, refers to a region in a template RNA having one or more sequence difference relative to the corresponding sequence in a target nucleic acid. The sequence difference may comprise, for example, a substitution, insertion, frameshift, or deletion.

The term “mutated” when applied to nucleic acid sequences means that nucleotides in a nucleic acid sequence are inserted, deleted, or changed compared to a reference (e.g., native) nucleic acid sequence. A single alteration may be made at a locus (a point mutation), or multiple nucleotides may be inserted, deleted, or changed at a single locus. In addition, one or more alterations may be made at any number of loci within a nucleic acid sequence. A nucleic acid sequence may be mutated by any method known in the art.

“Nucleic acid molecule” refers to both RNA and DNA molecules including, without limitation, complementary DNA (“cDNA”), genomic DNA (“gDNA”), and messenger RNA (“mRNA”), and also includes synthetic nucleic acid molecules, such as those that are chemically synthesized or recombinantly produced, such as RNA templates, as described herein. The nucleic acid molecule can be double-stranded or single-stranded, circular, or linear. If single-stranded, the nucleic acid molecule can be the sense strand or the antisense strand. Unless otherwise indicated, and as an example for all sequences described herein under the general format “SEQ ID NO:,” or “nucleic acid comprising SEQ ID NO: 1” refers to a nucleic acid, at least a portion which has either (i) the sequence of SEQ ID NO:1, or (ii) a sequence complimentary to SEQ ID NO:1. The choice between the two is dictated by the context in which SEQ ID NO: 1 is used. For instance, if the nucleic acid is used as a probe, the choice between the two is dictated by the requirement that the probe be complementary to the desired target. Nucleic acid sequences of the present disclosure may be modified chemically or biochemically or may contain non-natural or derivatized nucleotide bases, as will be readily appreciated by those of skill in the art. Such modifications include, for example, labels, methylation, substitution of one or more naturally occurring nucleotides with an analog, inter-nucleotide modifications such as uncharged linkages (for example, methyl phosphonates, phosphotriesters, phosphoramidates, carbamates, etc.), charged linkages (for example, phosphorothioates, phosphorodithioates, etc.), pendant moieties, (for example, polypeptides), intercalators (for example, acridine, psoralen, etc.), chelators, alkylators, and modified linkages (for example, alpha anomeric nucleic acids, etc.). Also included are chemically modified bases (see, for example, Table 13), backbones (see, for example, Table 14), and modified caps (see, for example, Table 15). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of a molecule, e.g., peptide nucleic acids (PNAs). Other modifications can include, for example, analogs in which the ribose ring contains a bridging moiety or other structure such as modifications found in “locked” nucleic acids (LNAs). In various embodiments, the nucleic acids are in operative association with additional genetic elements, such as tissue-specific expression-control sequence(s) (e.g., tissue-specific promoters and tissue-specific microRNA recognition sequences), as well as additional elements, such as inverted repeats (e.g., inverted terminal repeats, such as elements from or derived from viruses, e.g., AAV ITRs) and tandem repeats, inverted repeats/direct repeats, homology regions (segments with various degrees of homology to a target DNA), untranslated regions (UTRs) (5′, 3′, or both 5′ and 3′ UTRs), and various combinations of the foregoing. The nucleic acid elements of the systems provided by the invention can be provided in a variety of topologies, including single-stranded, double-stranded, circular, linear, linear with open ends, linear with closed ends, and particular versions of these, such as doggybone DNA (dbDNA), closed-ended DNA (ceDNA).

As used herein, a “gene expression unit” is a nucleic acid sequence comprising at least one regulatory nucleic acid sequence operably linked to at least one effector sequence. A first nucleic acid sequence is operably linked with a second nucleic acid sequence when the first nucleic acid sequence is placed in a functional relationship with the second nucleic acid sequence. For instance, a promoter or enhancer is operably linked to a coding sequence if the promoter or enhancer affects the transcription or expression of the coding sequence. Operably linked DNA sequences may be contiguous or non-contiguous. Where necessary to join two protein-coding regions, operably linked sequences may be in the same reading frame.

The terms “host genome” or “host cell”, as used herein, refer to a cell and/or its genome into which protein and/or genetic material has been introduced. It should be understood that such terms are intended to refer not only to the particular subject cell and/or genome, but to the progeny of such a cell and/or the genome of the progeny of such a cell. Because certain modifications may occur in succeeding generations due to either mutation or environmental influences, such progeny may not, in fact, be identical to the parent cell, but are still included within the scope of the term “host cell” as used herein. A host genome or host cell may be an isolated cell or cell line grown in culture, or genomic material isolated from such a cell or cell line, or may be a host cell or host genome which composing living tissue or an organism. In some instances, a host cell may be an animal cell or a plant cell, e.g., as described herein. In certain instances, a host cell may be a mammalian cell, a human cell, avian cell, reptilian cell, bovine cell, horse cell, pig cell, goat cell, sheep cell, chicken cell, or turkey cell. In certain instances, a host cell may be a corn cell, soy cell, wheat cell, or rice cell.

As used herein, “operative association” describes a functional relationship between two nucleic acid sequences, such as a 1) promoter and 2) a heterologous object sequence, and means, in such example, the promoter and heterologous object sequence (e.g., a gene of interest) are oriented such that, under suitable conditions, the promoter drives expression of the heterologous object sequence. For instance, a template nucleic acid carrying a promoter and a heterologous object sequence may be single-stranded, e.g., either the (+) or (−) orientation. An “operative association” between the promoter and the heterologous object sequence in this template means that, regardless of whether the template nucleic acid will be transcribed in a particular state, when it is in the suitable state (e.g., is in the (+) orientation, in the presence of required catalytic factors, and NTPs, etc.), it is accurately transcribed. Operative association applies analogously to other pairs of nucleic acids, including other tissue-specific expression control sequences (such as enhancers, repressors and microRNA recognition sequences), IR/DR, ITRs, UTRs, or homology regions and heterologous object sequences or sequences encoding a retroviral RT domain.

The term “primer binding site sequence” or “PBS sequence,” as used herein, refers to a portion of a template RNA capable of binding to a region comprised in a target nucleic acid sequence. In some instances, a PBS sequence is a nucleic acid sequence comprising at least 3, 4, 5, 6, 7, or 8 bases with 100% identity to the region comprised in the target nucleic acid sequence. In some embodiments the primer region comprises at least 5, 6, 7, 8 bases with 100% identity to the region comprised in the target nucleic acid sequence. Without wishing to be bound by theory, in some embodiments when a template RNA comprises a PBS sequence and a heterologous object sequence, the PBS sequence binds to a region comprised in a target nucleic acid sequence, allowing a reverse transcriptase domain to use that region as a primer for reverse transcription, and to use the heterologous object sequence as a template for reverse transcription.

As used herein, a “stem-loop sequence” refers to a nucleic acid sequence (e.g., RNA sequence) with sufficient self-complementarity to form a stem-loop, e.g., having a stem comprising at least two (e.g., 3, 4, 5, 6, 7, 8, 9, or 10) base pairs, and a loop with at least three (e.g., four) base pairs. The stem may comprise mismatches or bulges.

As used herein, a “tissue-specific expression-control sequence” means nucleic acid elements that increase or decrease the level of a transcript comprising the heterologous object sequence in a target tissue in a tissue-specific manner, e.g., preferentially in on-target tissue(s), relative to off-target tissue(s). In some embodiments, a tissue-specific expression-control sequence preferentially drives or represses transcription, activity, or the half-life of a transcript comprising the heterologous object sequence in the target tissue in a tissue-specific manner, e.g., preferentially in an on-target tissue(s), relative to an off-target tissue(s). Exemplary tissue-specific expression-control sequences include tissue-specific promoters, repressors, enhancers, or combinations thereof, as well as tissue-specific microRNA recognition sequences. Tissue specificity refers to on-target (tissue(s) where expression or activity of the template nucleic acid is desired or tolerable) and off-target (tissue(s) where expression or activity of the template nucleic acid is not desired or is not tolerable). For example, a tissue-specific promoter drives expression preferentially in on-target tissues, relative to off-target tissues. In contrast, a microRNA that binds the tissue-specific microRNA recognition sequences is preferentially expressed in off-target tissues, relative to on-target tissues, thereby reducing expression of a template nucleic acid in off-target tissues. Accordingly, a promoter and a microRNA recognition sequence that are specific for the same tissue, such as the target tissue, have contrasting functions (promote and repress, respectively, with concordant expression levels, i.e., high levels of the microRNA in off-target tissues and low levels in on-target tissues, while promoters drive high expression in on-target tissues and low expression in off-target tissues) with regard to the transcription, activity, or half-life of an associated sequence in that tissue.

### Introduction

This disclosure relates to methods and compositions for targeting, editing, modifying or manipulating a DNA sequence (e.g., inserting a heterologous object sequence into a target site of a mammalian genome) at one or more locations in a DNA sequence in a cell, tissue or subject, e.g., in vivo or in vitro. The heterologous object DNA sequence may include, e.g., a substitution, a deletion, an insertion, e.g., a coding sequence, a regulatory sequence, or a gene expression unit. More specifically, the disclosure provides methods for disrupting a TRAC gene and/or a B2M gene using reverse transcriptase-based systems for altering a genomic DNA sequence of interest, e.g., by inserting, deleting, or substituting one or more nucleotides into/from the sequence of interest.

The disclosure provides, in part, methods for disrupting a TRAC gene and/or a B2M gene using a heterologous gene modifying system comprising a heterologous gene modifying polypeptide component and a template nucleic acid (e.g., template RNA) component. In some embodiments, a heterologous gene modifying system can be used to introduce an alteration into a target site in a genome. In some embodiments, the heterologous gene modifying polypeptide component comprises a writing domain (e.g., a reverse transcriptase domain), a DNA-binding domain, and an endonuclease domain (e.g., nickase domain). In some embodiments, the template nucleic acid (e.g., template RNA) comprises a sequence (e.g., a gRNA spacer) that binds a target site in the genome (e.g., that binds to a second strand of the target site), a sequence (e.g., a gRNA scaffold) that binds the heterologous gene modifying polypeptide component, a heterologous object sequence, and a PBS sequence. Without wishing to be bound by theory, it is thought that the template nucleic acid (e.g., template RNA) binds to the second strand of a target site in the genome, and binds to the heterologous gene modifying polypeptide component (e.g., localizing the polypeptide component to the target site in the genome). It is thought that the endonuclease (e.g., nickase) of the heterologous gene modifying polypeptide component cuts the target site (e.g., the first strand of the target site), e.g., allowing the PBS sequence to bind to a sequence adjacent to the site to be altered on the first strand of the target site. It is thought that the writing domain (e.g., reverse transcriptase domain) of the polypeptide component uses the first strand of the target site that is bound to the complementary sequence comprising the PBS sequence of the template nucleic acid as a primer and the heterologous object sequence of the template nucleic acid as a template to, e.g., polymerize a sequence complementary to the heterologous object sequence. Without wishing to be bound by theory, it is thought that selection of an appropriate heterologous object sequence can result in substitution, deletion, and/or insertion of one or more nucleotides at the target site.

### Heterologous Gene Modifying Systems

In some embodiments, a heterologous gene modifying system described herein comprises: (A) a heterologous gene modifying polypeptide or a nucleic acid encoding the heterologous gene modifying polypeptide, wherein the heterologous gene modifying polypeptide comprises (i) a reverse transcriptase domain, and either (x) an endonuclease domain that contains DNA binding functionality or (y) an endonuclease domain and separate DNA binding domain; and (B) a template RNA. A heterologous gene modifying polypeptide, in some embodiments, acts as a substantially autonomous protein machine capable of integrating a template nucleic acid sequence into a target DNA molecule (e.g., in a mammalian host cell, such as a genomic DNA molecule in the host cell), substantially without relying on host machinery. For example, the heterologous gene modifying protein may comprise a DNA-binding domain, a reverse transcriptase domain, and an endonuclease domain. In some embodiments, the DNA-binding function may involve an RNA component that directs the protein to a DNA sequence, e.g., a gRNA spacer. In other embodiments, the heterologous gene modifying polypeptide may comprise a reverse transcriptase domain and an endonuclease domain. The RNA template element of a heterologous gene modifying system is typically heterologous to the heterologous gene modifying polypeptide element and provides an object sequence to be inserted (reverse transcribed) into the host genome. In some embodiments, the heterologous gene modifying polypeptide is capable of target primed reverse transcription. In some embodiments, the heterologous gene modifying polypeptide is capable of second-strand synthesis.

In some embodiments the heterologous gene modifying system is combined with a second polypeptide. In some embodiments, the second polypeptide may comprise an endonuclease domain. In some embodiments, the second polypeptide may comprise a polymerase domain, e.g., a reverse transcriptase domain. In some embodiments, the second polypeptide may comprise a DNA-dependent DNA polymerase domain. In some embodiments, the second polypeptide aids in completion of the genome edit, e.g., by contributing to second-strand synthesis or DNA repair resolution.

A functional heterologous gene modifying polypeptide can be made up of unrelated DNA binding, reverse transcription, and endonuclease domains. This modular structure allows combining of functional domains, e.g., dCas9 (DNA binding), MMLV reverse transcriptase (reverse transcription), FokI (endonuclease). In some embodiments, multiple functional domains may arise from a single protein, e.g., Cas9 or Cas9 nickase (DNA binding, endonuclease).

In some embodiments, a heterologous gene modifying polypeptide includes one or more domains that, collectively, facilitate 1) binding the template nucleic acid, 2) binding the target DNA molecule, and 3) facilitate integration of the at least a portion of the template nucleic acid into the target DNA. In some embodiments, the heterologous gene modifying polypeptide is an engineered polypeptide that comprises one or more amino acid substitutions to a corresponding naturally occurring sequence. In some embodiments, the heterologous gene modifying polypeptide comprises two or more domains that are heterologous relative to each other, e.g., through a heterologous fusion (or other conjugate) of otherwise wild-type domains, or well as fusions of modified domains, e.g., by way of replacement or fusion of a heterologous sub-domain or other substituted domain. For instance, in some embodiments, one or more of: the RT domain is heterologous to the DBD; the DBD is heterologous to the endonuclease domain; or the RT domain is heterologous to the endonuclease domain.

In some embodiments, a template RNA molecule for use in the system comprises, from 5′ to 3′ (1) a gRNA spacer; (2) a gRNA scaffold; (3) heterologous object sequence (4) a primer binding site (PBS) sequence. In some embodiments:
(1) Is a gRNA spacer of ~18-22 nt, e.g., is 20 nt
(2) Is a gRNA scaffold comprising one or more hairpin loops, e.g., 1, 2, of 3 loops for associating the template with a Cas domain, e.g., a nickase Cas9 domain. In some embodiments, the gRNA scaffold comprises the sequence, from 5′ to 3′, GTTTTAGAGCTAGAAATAGCAAGTTAAAATAAGGCTAGTCCGTTATCAACTT GAAAAAGTGGGACCGAGTCGGTCC (SEQ ID NO: 5008).
(3) In some embodiments, the heterologous object sequence is, e.g., 7-74, e.g., 10-20, 20-30, 30-40, 40-50, 50-60, 60-70, or 70-80 nt or, 80-90 nt in length. In some embodiments, the first (most 5′) base of the sequence is not C.
(4) In some embodiments, the PBS sequence that binds the target priming sequence after nicking occurs is e.g., 3-20 nt, e.g., 7-15 nt, e.g., 12-14 nt. In some embodiments, the PBS sequence has 40-60% GC content.

In some embodiments, a second gRNA associated with the system may help drive complete integration. In some embodiments, the second gRNA may target a location that is 0-200 nt away from the first-strand nick, e.g., 0-50, 50-100, 100-200 nt away from the first-strand nick. In some embodiments, the second gRNA can only bind its target sequence after the edit is made, e.g., the gRNA binds a sequence present in the heterologous object sequence, but not in the initial target sequence.

In some embodiments, a heterologous gene modifying system described herein is used to make an edit in HEK293, K562, U2OS, or HeLa cells. In some embodiment, a heterologous gene modifying system is used to make an edit in primary cells, e.g., primary cortical neurons from E18.5 mice.

In some embodiments, a heterologous gene modifying polypeptide as described herein comprises a reverse transcriptase or RT domain (e.g., as described herein) that comprises a MoMLV RT sequence or variant thereof. In embodiments, the MoMLV RT sequence comprises one or more mutations selected from D200N, L603W, T330P, T306K, W313F, D524G, E562Q, D583N, P51L, S67R, E67K, T197A, H204R, E302K, F309N, L435G, N454K, H594Q, D653N, R110S, and K103L. In embodiments, the MoMLV RT sequence comprises a combination of mutations, such as D200N, L603W, and T330P, optionally further including T306K and/or W313F.

In some embodiments, an endonuclease domain (e.g., as described herein) comprises nCas9, e.g., comprising an N863A mutation (e.g., in spCas9) or a H840A mutation.

In some embodiments, the heterologous object sequence (e.g., of a system as described herein) is about 1-50, 50-100, 100-200, 200-300, 300-400, 400-500, 500-600, 600-700, 700-800, 800-900, 900-1000, or more, nucleotides in length.

In some embodiments, the RT and endonuclease domains are joined by a flexible linker, e.g., comprising the amino acid sequence SGGSSGGSSGSETPGTSESATPESSGGSSGGSS (SEQ ID NO: 5006).

In some embodiments, the endonuclease domain is N-terminal relative to the RT domain. In some embodiments, the endonuclease domain is C-terminal relative to the RT domain.

In some embodiments, the system incorporates a heterologous object sequence into a target site by TPRT, e.g., as described herein.

In some embodiments, a heterologous gene modifying polypeptide comprises a DNA binding domain. In some embodiments, a heterologous gene modifying polypeptide comprises an RNA binding domain. In some embodiments, the RNA binding domain comprises an RNA binding domain of B-box protein, MS2 coat protein, dCas, or an element of a sequence of a table herein. In some embodiments, the RNA binding domain is capable of binding to a template RNA with greater affinity than a reference RNA binding domain.

In some embodiments, a heterologous gene modifying system is capable of producing an insertion into the target site of at least 45, 50, 55, 60, 65, 70, 75, 80, 85, 90, 95, or 100 nucleotides (and optionally no more than 500, 400, 300, 200, or 100 nucleotides). In some embodiments, a heterologous gene modifying system is capable of producing an insertion into the target site of at least 1, 2, 3, 4, 5, 6, 7, 8, 9, 10, 15, 20, 25, 30, 35, 40, 45, 50, 55, 60, 65, 70, 75, 80, 85, 90, 95, or 100 nucleotides (and optionally no more than 500, 400, 300, 200, or 100 nucleotides). In some embodiments, a heterologous gene modifying system is capable of producing an insertion into the target site of at least 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10 kilobases (and optionally no more than 1, 5, 10, or 20 kilobases). In some embodiments, a heterologous gene modifying system is capable of producing a deletion of at least 81, 85, 90, 95, 100, 110, 120, 130, 140, 150, 160, 170, 180, 190, or 200 nucleotides (and optionally no more than 500, 400, 300, or 200 nucleotides). In some embodiments, a heterologous gene modifying system is capable of producing a deletion of at least 81, 85, 90, 95, 100, 110, 120, 130, 140, 150, 160, 170, 180, 190, or 200 nucleotides (and optionally no more than 500, 400, 300, or 200 nucleotides). In some embodiments, a heterologous gene modifying system is capable of producing a deletion of at least 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200 nucleotides (and optionally no more than 500, 400, 300, or 200 nucleotides). In some embodiments, a heterologous gene modifying system is capable of producing a deletion of at least 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10 kilobases (and optionally no more than 1, 5, 10, or 20 kilobases). In some embodiments, a heterologous gene modifying system is capable of producing a substitution into the target site of at least 1, 2, 3, 4, 5, 6, 7, 8, 9, 10, 15, 20, 25, 30, 35, 40, 45, 50, 60, 70, 80, 90, or 100 or more nucleotides. In some embodiments, a heterologous gene modifying system is capable of producing a substitution in the target site of 1-2, 2-3, 3-4, 4-5, 5-10, 10-15, 15-20, 20-30, 30-40, 40-50, 50-60, 60-70, 70-80, 80-90, or 90-100 nucleotides.

In some embodiments, the substitution is a transition mutation. In some embodiments, the substitution is a transversion mutation. In some embodiments, the substitution converts an adenine to a thymine, an adenine to a guanine, an adenine to a cytosine, a guanine to a thymine, a guanine to a cytosine, a guanine to an adenine, a thymine to a cytosine, a thymine to an adenine, a thymine to a guanine, a cytosine to an adenine, a cytosine to a guanine, or a cytosine to a thymine.

In some embodiments, an insertion, deletion, substitution, or combination thereof, increases or decreases expression (e.g. transcription or translation) of a gene. In some embodiments, an insertion, deletion, or combination thereof disrupts a gene. In some embodiments, an insertion, deletion, substitution, or combination thereof, increases or decreases expression (e.g. transcription or translation) of a gene by altering, adding, or deleting sequences in a promoter or enhancer, e.g. sequences that bind transcription factors. In some embodiments, an insertion, deletion, substitution, or combination thereof alters translation of a gene (e.g. alters an amino acid sequence), inserts or deletes a start or stop codon, alters or fixes the translation frame of a gene. In some embodiments, an insertion, deletion, substitution, or combination thereof alters splicing of a gene, e.g. by inserting, deleting, or altering a splice acceptor or donor site. In some embodiments, an insertion, deletion, substitution, or combination thereof alters transcript or protein half-life. In some embodiments, an insertion, deletion, substitution, or combination thereof alters protein localization in the cell (e.g. from the cytoplasm to a mitochondria, from the cytoplasm into the extracellular space (e.g. adds a secretion tag)). In some embodiments, an insertion, deletion, substitution, or combination thereof alters (e.g. improves) protein folding (e.g. to prevent accumulation of misfolded proteins). In some embodiments, an insertion, deletion, substitution, or combination thereof, alters, increases, decreases the activity of a gene, e.g. a protein encoded by the gene.

Exemplary heterologous gene modifying polypeptides, and systems comprising them and methods of using them are described, e.g., in PCT/US2021/020948, which is incorporated herein by reference with respect to retroviral RT domains, including the amino acid and nucleic acid sequences therein.

Exemplary heterologous gene modifying polypeptides and retroviral RT domain sequences are also described, e.g., in International Application No. PCT/US21/20948 filed Mar. 4, 2021, e.g., at Table 30, Table 31, and Table 44 therein; the entire application is incorporated by reference herein with respect to retroviral RTs, e.g., in said sequences and tables. Accordingly, a heterologous gene modifying polypeptide described herein may comprise an amino acid sequence according to any of the Tables mentioned in this paragraph, or a domain thereof (e.g., a retroviral RT domain), or a functional fragment or variant of any of the foregoing, or an amino acid sequence having at least 70%, 80%, 85%, 90%, 95%, or 99% identity thereto.

In some embodiments, a polypeptide for use in any of the systems described herein can be a molecular reconstruction or ancestral reconstruction based upon the aligned polypeptide sequence of multiple homologous proteins. In some embodiments, a reverse transcriptase domain for use in any of the systems described herein can be a molecular reconstruction or an ancestral reconstruction, or can be modified at particular residues, based upon alignments of reverse transcriptase domains from the same or different sources. A skilled artisan can, based on the Accession numbers provided herein, align polypeptides or nucleic acid sequences, e.g., by using routine sequence analysis tools as Basic Local Alignment Search Tool (BLAST) or CD-Search for conserved domain analysis. Molecular reconstructions can be created based upon sequence consensus, e.g. using approaches described in Ivics et al., Cell 1997, 501-510; Wagstaff et al., Molecular Biology and Evolution 2013, 88-99.

### Polypeptide Components of Heterologous Gene Modifying Systems

In some embodiments, the heterologous gene modifying polypeptide possesses the functions of DNA target site binding, template nucleic acid (e.g., RNA) binding, DNA target site cleavage, and template nucleic acid (e.g., RNA) writing, e.g., reverse transcription. In some embodiments, each functions is contained within a distinct domain. In some embodiments, a function may be attributed to two or more domains (e.g., two or more domains, together, exhibit the functionality). In some embodiments, two or more domains may have the same or similar function (e.g., two or more domains each independently have DNA-binding functionality, e.g., for two different DNA sequences). In other embodiments, one or more domains may be capable of enabling one or more functions, e.g., a Cas9 domain enabling both DNA binding and target site cleavage. In some embodiments, the domains are all located within a single polypeptide. In some embodiments, a first domain is in one polypeptide and a second domain is in a second polypeptide. For example, in some embodiments, the sequences may be split between a first polypeptide and a second polypeptide, e.g., wherein the first polypeptide comprises a reverse transcriptase (RT) domain and wherein the second polypeptide comprises a DNA-binding domain and an endonuclease domain, e.g., a nickase domain. As a further example, in some embodiments, the first polypeptide and the second polypeptide each comprise a DNA binding domain (e.g., a first DNA binding domain and a second DNA binding domain). In some embodiments, the first and second polypeptide may be brought together post-translationally via a split-intein to form a single heterologous gene modifying polypeptide.

In some aspects, a heterologous gene modifying polypeptide described herein comprises (e.g., a system described herein comprises a heterologous gene modifying polypeptide that comprises): 1) a Cas domain (e.g., a Cas nickase domain, e.g., a Cas9 nickase domain); 2) a reverse transcriptase (RT) domain of Table D, or a sequence having at least 70%, 75%, 80%, 85%, 90%, 95%, 97%, 98%, or 99% identity thereto, wherein the RT domain is C-terminal of the Cas domain; and a linker disposed between the RT domain and the Cas domain, wherein the linker has a sequence from the same row of Table D as the RT domain, or a sequence having at least 70%, 75%, 80%, 85%, 90%, 95%, 97%, 98%, or 99% identity thereto.

In some embodiments, the RT domain has a sequence with 100% identity to the RT domain of Table D and the linker has a sequence with 100% identity to the linker sequence from the same row of Table D as the RT domain. In some embodiments, the Cas domain comprises a sequence of Table 8, or a sequence having at least 70%, 75%, 80%, 85%, 90%, 95%, 98%, or 99% identity thereto. In some embodiments, the heterologous gene modifying polypeptide comprises an amino acid sequence according to any of SEQ ID NOs: 1-3332 in the sequence listing, or a sequence having at least 70%, 75%, 80%, 85%, 90%, 95%, 97%, 98%, or 99% identity thereto.

In some embodiments, the heterologous gene modifying polypeptide comprises a GG amino acid sequence between the Cas domain and the linker, an AG amino acid sequence between the RT domain and the second NLS, and/or a GG amino acid sequence between the linker and the RT domain. In some embodiments, the heterologous gene modifying polypeptide comprises a sequence of SEQ ID NO: 4000 which comprises the first NLS and the Cas domain, or a sequence having at least 70%, 75%, 80%, 85%, 90%, 95%, 98%, or 99% identity thereto. In some embodiments, the heterologous gene modifying polypeptide comprises a sequence of SEQ ID NO: 4001 which comprises the second NLS, or a sequence having at least 70%, 75%, 80%, 85%, 90%, 95%, 98%, or 99% identity thereto.

Exemplary N-terminal NLS-Cas9 domain
(SEQ ID NO: 4000)
MPAAKRVKLDGGDKKYSIGLDIGTNSVGWAVITDEYKVPSKKFKVLGNTD
RHSIKKNLIGALLFDSGETAEATRLKRTARRRYTRRKNRICYLQEIFSNE
MAKVDDSFFHRLEESFLVEEDKKHERHPIFGNIVDEVAYHEKYPTIYHLR
KKLVDSTDKADLRLIYLALAHMIKFRGHFLIEGDLNPDNSDVDKLFIQLV
QTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADL
FLAAKNLSDAILLSDILRVNTEITKAPLSASMIKRYDEHHQDLTLLKALV
RQQLPEKYKEIFFDQSKNGYAGYIDGGASQEEFYKFIKPILEKMDGTEEL
LVKLNREDLLRKORTFDNGSIPHQIHLGELHAILRRQEDFYPFLKDNREK
IEKILTFRIPYYVGPLARGNSRFAWMTRKSEETITPWNFEEVVDKGASAQ
SFIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFNA
SLGTYHDLLKIIKDKDFLDNEENEDILEDIVLTLTLFEDREMIEERLKTY
AHLFDDKVMKQLKRRRYTGWGRLSRKLINGIRDKQSGKTILDFLKSDGFA
NRNFMQLIHDDSLTFKEDIQKAQVSGQGDSLHEHIANLAGSPAIKKGILQ
TVKVVDELVKVMGRHKPENIVIEMARENOTTQKGQKNSRERMKRIEEGIK
ELGSQILKEHPVENTQLQNEKLYLYYLONGRDMYVDQELDINRLSDYDVD
HIVPQSFLKDDSIDNKVLTRSDKARGKSDNVPSEEVVKKMKNYWRQLLNA
KLITQRKFDNLTKAERGGLSELDKAGFIKRQLVETRQITKHVAQILDSRM
NTKYDENDKLIREVKVITLKSKLVSDFRKDFQFYKVREINNYHHAHDAYL
NAVVGTALIKKYPKLESEFVYGDYKVYDVRKMIAKSEQEIGKATAKYFFY
SNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTV
AYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLA
SHYEKLKGSPEDNEQKQLFVEQHKHYLDEIIEQISEFSKRVILADANLDK
VLSAYNKHRDKPIREQAENIIHLFTLTNLGAPAAFKYFDTTIDRKRYTST
KEVLDATLIHQSITGLYETRIDLSQLGGDGG
Exemplary C-terminal sequence comprising
an NLS
(SEQ ID NO: 4001)
AGKRTADGSEFEKRTADGSEFESPKKKAKVE

### Writing Domain (RT Domain)

In certain aspects of the present invention, the writing domain of the heterologous gene modifying system possesses reverse transcriptase activity and is also referred to as a reverse transcriptase domain (a RT domain). In some embodiments, the RT domain comprises an RT catalytic portion and RNA-binding region (e.g., a region that binds the template RNA).

In some embodiments, a nucleic acid encoding the reverse transcriptase is altered from its natural sequence to have altered codon usage, e.g. improved for human cells. In some embodiments the reverse transcriptase domain is a heterologous reverse transcriptase from a retrovirus. In some embodiments, the RT domain comprising a heterologous gene modifying polypeptide has been mutated from its original amino acid sequence, e.g., has at least 1, 2, 3, 4, 5, 6, 7, 8, 9, 10, 20, 30, 40, 50, 60, 70, 80, 90, or 100 substitutions. In some embodiments, the RT domain is derived from the RT of a retrovirus, e.g., HIV-1 RT, Moloney Murine Leukemia Virus (MMLV) RT, avian myeloblastosis virus (AMV) RT, or Rous Sarcoma Virus (RSV) RT.

In some embodiments, the retroviral reverse transcriptase (RT) domain exhibits enhanced stringency of target-primed reverse transcription (TPRT) initiation, e.g., relative to an endogenous RT domain. In some embodiments, the RT domain initiates TPRT when the 3 nt in the target site immediately upstream of the first strand nick, e.g., the genomic DNA priming the RNA template, have at least 66% or 100% complementarity to the 3 nt of homology in the RNA template. In some embodiments, the RT domain initiates TPRT when there are less than 5 nt mismatched (e.g., less than 1, 2, 3, 4, or 5 nt mismatched) between the template RNA homology and the target DNA priming reverse transcription. In some embodiments, the RT domain is modified such that the stringency for mismatches in priming the TPRT reaction is increased, e.g., wherein the RT domain does not tolerate any mismatches or tolerates fewer mismatches in the priming region relative to a wild-type (e.g., unmodified) RT domain. In some embodiments, the RT domain comprises a HIV-1 RT domain. In embodiments, the HIV-1 RT domain initiates lower levels of synthesis even with three nucleotide mismatches relative to an alternative RT domain (e.g., as described by Jamburuthugoda and Eickbush J Mol Biol 407 (5): 661-672 (2011); incorporated herein by reference in its entirety). In some embodiments, the RT domain forms a dimer (e.g., a heterodimer or homodimer). In some embodiments, the RT domain is monomeric.

In some embodiments, an RT domain, naturally functions as a monomer or as a dimer (e.g., heterodimer or homodimer). In some embodiments, an RT domain naturally functions as a monomer, e.g., is derived from a virus wherein it functions as a monomer. In embodiments, the RT domain is selected from an RT domain from murine leukemia virus (MLV; sometimes referred to as MoMLV) (e.g., P03355), porcine endogenous retrovirus (PERV) (e.g., UniProt Q4VFZ2), mouse mammary tumor virus (MMTV) (e.g., UniProt P03365), Avian reticuloendotheliosis virus (AVIRE) (e.g., UniProtKB accession: P03360); Feline leukemia virus (FLV or FeLV) (e.g., e.g., UniProtKB accession: P10273); Mason-Pfizer monkey virus (MPMV) (e.g., UniProt P07572), bovine leukemia virus (BLV) (e.g., UniProt P03361), human T-cell leukemia virus-1 (HTLV-1) (e.g., UniProt P03362), human foamy virus (HFV) (e.g., UniProt P14350), simian foamy virus (SFV) (e.g., SFV3L) (e.g., UniProt P23074 or P27401), or bovine foamy/syncytial virus (BFV/BSV) (e.g., UniProt 041894), or a functional fragment or variant thereof (e.g., an amino acid sequence having at least 70%, 80%, 90%, 95%, or 99% identity thereto). In some embodiments, an RT domain is dimeric in its natural functioning. In some embodiments, the RT domain is derived from a virus wherein it functions as a dimer. In embodiments, the RT domain is selected from an RT domain from avian sarcoma/leukemia virus (ASLV) (e.g., UniProt A0A142BKH1), Rous sarcoma virus (RSV) (e.g., UniProt P03354), avian myeloblastosis virus (AMV) (e.g., UniProt Q83133), human immunodeficiency virus type I (HIV-1) (e.g., UniProt P03369), human immunodeficiency virus type II (HIV-2) (e.g., UniProt P15833), simian immunodeficiency virus (SIV) (e.g., UniProt P05896), bovine immunodeficiency virus (BIV) (e.g., UniProt P19560), equine infectious anemia virus (EIAV) (e.g., UniProt P03371), or feline immunodeficiency virus (FIV) (e.g., UniProt P16088) (Herschhorn and Hizi Cell Mol Life Sci 67 (16): 2717-2747 (2010)), or a functional fragment or variant thereof (e.g., an amino acid sequence having at least 70%, 80%, 90%, 95%, or 99% identity thereto). Naturally heterodimeric RT domains may, in some embodiments, also be functional as homodimers. In some embodiments, dimeric RT domains are expressed as fusion proteins, e.g., as homodimeric fusion proteins or heterodimeric fusion proteins. In some embodiments, the RT function of the system is fulfilled by multiple RT domains (e.g., as described herein). In further embodiments, the multiple RT domains are fused or separate, e.g., may be on the same polypeptide or on different polypeptides.

In some embodiments, a heterologous gene modifying system described herein comprises an integrase domain, e.g., wherein the integrase domain may be part of the RT domain. In some embodiments, an RT domain (e.g., as described herein) comprises an integrase domain. In some embodiments, an RT domain (e.g., as described herein) lacks an integrase domain, or comprises an integrase domain that has been inactivated by mutation or deleted. In some embodiment, a heterologous gene modifying system described herein comprises an RNase H domain, e.g., wherein the RNase H domain may be part of the RT domain. In some embodiments, the RNase H domain is not part of the RT domain and is covalently linked via a flexible linker. In some embodiments, an RT domain (e.g., as described herein) comprises an RNase H domain, e.g., an endogenous RNAse H domain or a heterologous RNase H domain. In some embodiments, an RT domain (e.g., as described herein) lacks an RNase H domain. In some embodiments, an RT domain (e.g., as described herein) comprises an RNase H domain that has been added, deleted, mutated, or swapped for a heterologous RNase H domain. In some embodiments, the polypeptide comprises an inactivated endogenous RNase H domain. In some embodiments, an endogenous RNase H domain from one of the other domains of the polypeptide is genetically removed such that it is not included in the polypeptide, e.g., the endogenous RNase H domain is partially or completely truncated from the comprising domain. In some embodiments, mutation of an RNase H domain yields a polypeptide exhibiting lower RNase activity, e.g., as determined by the methods described in Kotewicz et al. Nucleic Acids Res 16 (1): 265-277 (1988) (incorporated herein by reference in its entirety), e.g., lower by at least 10%, 20%, 30%, 40%, 50%, 60%, 70%, 80%, or 90% compared to an otherwise similar domain without the mutation. In some embodiments, RNase H activity is abolished.

In some embodiments, an RT domain is mutated to increase fidelity compared to an otherwise similar domain without the mutation. For instance, in some embodiments, a YADD (SEQ ID NO: 37635) or YMDD motif (SEQ ID NO: 37636) in an RT domain (e.g., in a reverse transcriptase) is replaced with YVDD (SEQ ID NO: 37637). In embodiments, replacement of the YADD (SEQ ID NO: 37635) or YMDD (SEQ ID NO: 37636) or YVDD (SEQ ID NO: 37637) results in higher fidelity in retroviral reverse transcriptase activity (e.g., as described in Jamburuthugoda and Eickbush J Mol Biol 2011; incorporated herein by reference in its entirety).

In some embodiments, a heterologous gene modifying polypeptide described herein comprises an RT domain having an amino acid sequence according to Table 6, or a sequence having at least 70%, 80%, 85%, 90%, 95%, 97%, 98%, or 99% identity thereto. In some embodiments, a nucleic acid described herein encodes an RT domain having an amino acid sequence according to Table 6, or a sequence having at least 70%, 80%, 85%, 90%, 95%, 97%, 98%, or 99% identity thereto.

TABLE 6
Exemplary reverse transcriptase domains from retroviruses

| RT | SEQ ID | |
|---|---|---|
| Name | NO: | RT amino acid sequence |
| AVIRE_ | 8,001 | TAPLEEEYRLFLEAPIQNVTLLEQWKREIPKVWAEINPPGLASTQAPIHVQLLSTALPVRVRQYPITLEAKRSL |
| P03360 | | RETIRKFRAAGILRPVHSPWNTPLLPVRKSGTSEYRMVQDLREVNKRVETIHPTVPNPYTLLSLLPPDRIWYSV |
| | | LDLKDAFFCIPLAPESQLIFAFEWADAEEGESGQLTWTRLPQGFKNSPTLFDEALNRDLQGFRLDHPSVSLLQY |
| | | VDDLLIAADTQAACLSATRDLLMTLAELGYRVSGKKAQLCQEEVTYLGFKIHKGSRSLSNSRTQAILQIPVPKT |
| | | KRQVREFLGTIGYCRLWIPGFAELAQPLYAATRGGNDPLVWGEKEEEAFQSLKLALTQPPALALPSLDKPFQLF |
| | | VEETSGAAKGVLTQALGPWKRPVAYLSKRLDPVAAGWPRCLRAIAAAALLTREASKLTFGQDIEITSSHNLESL |
| | | LRSPPDKWLTNARITQYQVLLLDPPRVRFKQTAALNPATLLPETDDTLPIHHCLDTLDSLTSTRPDLTDQPLAQ |
| | | AEATLFTDGSSYIRDGKRYAGAAVVTLDSVIWAEPLPIGTSAQKAELIALTKALEWSKDKSVNIYTDSRYAFAT |
| | | LHVHGMIYRERGLLTAGGKAIKNAPEILALLTAVWLPKRVAVMHCKGHQKDDAPTSTGNRRADEVAREVAIRPL |
| | | STQATIS |
| AVIRE_ | 8,002 | TAPLEEEYRLFLEAPIQNVTLLEQWKREIPKVWAEINPPGLASTQAPIHVQLLSTALPVRVRQYPITLEAKRSL |
| P03360_ | | RETIRKFRAAGILRPVHSPWNTPLLPVRKSGTSEYRMVQDLREVNKRVETIHPTVPNPYTLLSLLPPDRIWYSV |
| 3mut | | LDLKDAFFCIPLAPESQLIFAFEWADAEEGESGQLTWTRLPQGFKNSPTLFNEALNRDLQGFRLDHPSVSLLQY |
| | | VDDLLIAADTQAACLSATRDLLMTLAELGYRVSGKKAQLCQEEVTYLGFKIHKGSRSLSNSRTQAILQIPVPKT |
| | | KRQVREFLGTIGYCRLWIPGFAELAQPLYAATRPGNDPLVWGEKEEEAFQSLKLALTQPPALALPSLDKPFQLF |
| | | VEETSGAAKGVLTQALGPWKRPVAYLSKRLDPVAAGWPRCLRAIAAAALLTREASKLTFGQDIEITSSHNLESL |
| | | LRSPPDKWLTNARITQYQVLLLDPPRVRFKQTAALNPATLLPETDDTLPIHHCLDTLDSLTSTRPDLTDQPLAQ |
| | | AEATLFTDGSSYIRDGKRYAGAAVVTLDSVIWAEPLPIGTSAQKAELIALTKALEWSKDKSVNIYTDSRYAFAT |
| | | LHVHGMIYRERGWLTAGGKAIKNAPEILALLTAVWLPKRVAVMHCKGHQKDDAPTSTGNRRADEVAREVAIRPL |
| | | STQATIS |
| AVIRE_ | 8,003 | TAPLEEEYRLFLEAPIQNVTLLEQWKREIPKVWAEINPPGLASTQAPIHVQLLSTALPVRVRQYPITLEAKRSL |
| P03360_ | | RETIRKFRAAGILRPVHSPWNTPLLPVRKSGTSEYRMVQDLREVNKRVETIHPTVPNPYTLLSLLPPDRIWYSV |
| 3mutA | | LDLKDAFFCIPLAPESQLIFAFEWADAEEGESGQLTWTRLPQGFKNSPTLFNEALNRDLQGFRLDHPSVSLLQY |
| | | VDDLLIAADTQAACLSATRDLLMTLAELGYRVSGKKAQLCQEEVTYLGFKIHKGSRSLSNSRTQAILQIPVPKT |
| | | KRQVREFLGKIGYCRLFIPGFAELAQPLYAATRPGNDPLVWGEKEEEAFQSLKLALTQPPALALPSLDKPFQLF |
| | | VEETSGAAKGVLTQALGPWKRPVAYLSKRLDPVAAGWPRCLRAIAAAALLTREASKLTFGQDIEITSSHNLESL |
| | | LRSPPDKWLTNARITQYQVLLLDPPRVRFKQTAALNPATLLPETDDTLPIHHOLDTLDSLTSTRPDLTDQPLAQ |
| | | AEATLFTDGSSYIRDGKRYAGAAVVTLDSVIWAEPLPIGTSAQKAELIALTKALEWSKDKSVNIYTDSRYAFAT |
| | | LHVHGMIYRERGWLTAGGKAIKNAPEILALLTAVWLPKRVAVMHCKGHQKDDAPTSTGNRRADEVAREVAIRPL |
| | | STQATIS |
| BAEVM_ | 8,004 | TVSLQDEHRLFDIPVTTSLPDVWLQDFPQAWAETGGLGRAKCQAPIIIDLKPTAVPVSIKQYPMSLEAHMGIRQ |
| P10272 | | HIIKFLELGVLRPCRSPWNTPLLPVKKPGTQDYRPVQDLREINKRTVDIHPTVPNPYNLLSTLKPDYSWYTVLD |
| | | LKDAFFCLPLAPQSQELFAFEWKDPERGISGQLTWTRLPQGFKNSPTLFDEALHRDLTDFRTQHPEVTLLQYVD |
| | | DLLLAAPTKKACTQGTRHLLQELGEKGYRASAKKAQICQTKVTYLGYILSEGKRWLTPGRIETVARIPPPRNPR |
| | | EVREFLGTAGFCRLWIPGFAELAAPLYALTKESTPFTWQTEHQLAFEALKKALLSAPALGLPDTSKPFTLFLDE |
| | | RQGIAKGVLTQKLGPWKRPVAYLSKKLDPVAAGWPPCLRIMAATAMLVKDSAKLTLGQPLTVITPHTLEAIVRQ |
| | | PPDRWITNARLTHYQALLLDTDRVQFGPPVTLNPATLLPVPENQPSPHDCRQVLAETHGTREDLKDQELPDADH |
| | | TWYTDGSSYLDSGTRRAGAAVVDGHNTIWAQSLPPGTSAQKAELIALTKALELSKGKKANIYTDSRYAFATAHT |
| | | HGSIYERRGLLTSEGKEIKNKAEIIALLKALFLPQEVAIIHCPGHQKGQDPVAVGNRQADRVARQAAMAEVLTL |
| | | ATEPDNTSHIT |
| BAEVM | 8,005 | TVSLQDEHRLFDIPVTTSLPDVWLQDFPQAWAETGGLGRAKCQAPIIIDLKPTAVPVSIKQYPMSLEAHMGIRQ |
| P10272_ | | HIIKFLELGVLRPCRSPWNTPLLPVKKPGTQDYRPVQDLREINKRTVDIHPTVPNPYNLLSTLKPDYSWYTVLD |
| 3mut | | LKDAFFCLPLAPQSQELFAFEWKDPERGISGQLTWTRLPQGFKNSPTLFNEALHRDLTDFRTQHPEVTLLQYVD |
| | | DLLLAAPTKKACTQGTRHLLQELGEKGYRASAKKAQICQTKVTYLGYILSEGKRWLTPGRIETVARIPPPRNPR |
| | | EVREFLGTAGFCRLWIPGFAELAAPLYALTKPSTPFTWQTEHQLAFEALKKALLSAPALGLPDTSKPFTLFLDE |
| | | RQGIAKGVLTQKLGPWKRPVAYLSKKLDPVAAGWPPCLRIMAATAMLVKDSAKLTLGQPLTVITPHTLEAIVRQ |
| | | PPDRWITNARLTHYQALLLDTDRVQFGPPVTLNPATLLPVPENQPSPHDCRQVLAETHGTREDLKDQELPDADH |
| | | TWYTDGSSYLDSGTRRAGAAVVDGHNTIWAQSLPPGTSAQKAELIALTKALELSKGKKANIYTDSRYAFATAHT |
| | | HGSIYERRGWLTSEGKEIKNKAEIIALLKALFLPQEVAIIHCPGHQKGQDPVAVGNRQADRVARQAAMAEVLTL |
| | | ATEPDNTSHIT |
| BAEVM_ | 8,006 | TVSLQDEHRLFDIPVTTSLPDVWLQDFPQAWAETGGLGRAKCQAPIIIDLKPTAVPVSIKQYPMSLEAHMGIRQ |
| P10272_ | | HIIKFLELGVLRPCRSPWNTPLLPVKKPGTQDYRPVQDLREINKRTVDIHPTVPNPYNLLSTLKPDYSWYTVLD |
| 3mutA | | LKDAFFCLPLAPQSQELFAFEWKDPERGISGQLTWTRLPQGFKNSPTLFNEALHRDLTDFRTQHPEVTLLQYVD |
| | | DLLLAAPTKKACTQGTRHLLQELGEKGYRASAKKAQICQTKVTYLGYILSEGKRWLTPGRIETVARIPPPRNPR |
| | | EVREFLGKAGFCRLFIPGFAELAAPLYALTKPSTPFTWQTEHQLAFEALKKALLSAPALGLPDTSKPFTLFLDE |
| | | RQGIAKGVLTQKLGPWKRPVAYLSKKLDPVAAGWPPCLRIMAATAMLVKDSAKLTLGQPLTVITPHTLEAIVRQ |
| | | PPDRWITNARLTHYQALLLDTDRVQFGPPVTLNPATLLPVPENQPSPHDCRQVLAETHGTREDLKDQELPDADH |
| | | TWYTDGSSYLDSGTRRAGAAVVDGHNTIWAQSLPPGTSAQKAELIALTKALELSKGKKANIYTDSRYAFATAHT |
| | | HGSIYERRGWLTSEGKEIKNKAEIIALLKALFLPQEVAIIHCPGHQKGQDPVAVGNRQADRVARQAAMAEVLTL |
| | | ATEPDNTSHIT |
| BLVAU_ | 8,007 | GVLDAPPSHIGLEHLPPPPEVPQFPLNLERLQALQDLVHRSLEAGYISPWDGPGNNPVFPVRKPNGAWRFVHDL |
| P25059 | | RVTNALTKPIPALSPGPPDLTAIPTHLPHIICLDLKDAFFQIPVEDRFRSYFAFTLPTPGGLQPHRRFAWRVLP |
| | | QGFINSPALFERALQEPLRQVSAAFSQSLLVSYMDDILYVSPTEEQRLQCYQTMAAHLRDLGFQVASEKTRQTP |
| | | SPVPFLGQMVHERMVTYQSLPTLQISSPISLHQLQTVLGDLQWVSRGTPTTRRPLQLLYSSLKGIDDPRAIIHL |
| | | SPEQQQGIAELRQALSHNARSRYNEQEPLLAYVHLTRAGSTLVLFQKGAQFPLAYFQTPLTDNQASPWGLLLLL |
| | | GCQYLQAQALSSYAKTILKYYHNLPKTSLDNWIQSSEDPRVQELLQLWPQISSQGIQPPGPWKTLVTRAEVFLT |
| | | PQFSPEPIPAALCLFSDGAARRGAYCLWKDHLLDFQAVPAPESAQKGELAGLLAGLAAAPPEPLNIWVDSKYLY |
| | | SLLRTLVLGAWLQPDPVPSYALLYKSLLRHPAIFVGHVRSHSSASHPIASLNNYVDQL |
| BLVAU | 8,008 | GVLDAPPSHIGLEHLPPPPEVPQFPLNLERLQALQDLVHRSLEAGYISPWDGPGNNPVFPVRKPNGAWRFVHDL |
| P25059_ | | RVTNALTKPIPALSPGPPDLTAIPTHLPHIICLDLKDAFFQIPVEDRFRSYFAFTLPTPGGLQPHRRFAWRVLP |
| 2mut | | QGFINSPALFQRALQEPLRQVSAAFSQSLLVSYMDDILYVSPTEEQRLQCYQTMAAHLRDLGFQVASEKTRQTP |
| | | SPVPFLGQMVHERMVTYQSLPTLQISSPISLHQLQTVLGDLQWVSRGTPTTRRPLQLLYSSLKPIDDPRAIIHL |
| | | SPEQQQGIAELRQALSHNARSRYNEQEPLLAYVHLTRAGSTLVLFQKGAQFPLAYFQTPLTDNQASPWGLLLLL |
| | | GCQYLQAQALSSYAKTILKYYHNLPKTSLDNWIQSSEDPRVQELLQLWPQISSQGIQPPGPWKTLVTRAEVFLT |
| | | PQFSPEPIPAALCLFSDGAARRGAYCLWKDHLLDFQAVPAPESAQKGELAGLLAGLAAAPPEPLNIWVDSKYLY |
| | | SLLRTLVLGAWLQPDPVPSYALLYKSLLRHPAIFVGHVRSHSSASHPIASLNNYVDQL |
| BLVJ_ | 8,009 | GVLDTPPSHIGLEHLPPPPEVPQFPLNLERLQALQDLVHRSLEAGYISPWDGPGNNPVFPVRKPNGAWRFVHDL |
| P03361 | | RATNALTKPIPALSPGPPDLTAIPTHPPHIICLDLKDAFFQIPVEDRFRFYLSFTLPSPGGLQPHRRFAWRVLP |
| | | QGFINSPALFERALQEPLRQVSAAFSQSLLVSYMDDILYASPTEEQRSQCYQALAARLRDLGFQVASEKTSQTP |
| | | SPVPFLGQMVHEQIVTYQSLPTLQISSPISLHQLQAVLGDLQWVSRGTPTTRRPLQLLYSSLKRHHDPRAIIQL |
| | | SPEQLQGIAELRQALSHNARSRYNEQEPLLAYVHLTRAGSTLVLFQKGAQFPLAYFQTPLTDNQASPWGLLLLL |
| | | GCQYLQTQALSSYAKPILKYYHNLPKTSLDNWIQSSEDPRVQELLQLWPQISSQGIQPPGPWKTLITRAEVFLT |
| | | PQFSPDPIPAALCLFSDGATGRGAYCLWKDHLLDFQAVPAPESAQKGELAGLLAGLAAAPPEPVNIWVDSKYLY |
| | | SLLRTLVLGAWLQPDPVPSYALLYKSLLRHPAIVVGHVRSHSSASHPIASLNNYVDQL |
| BLVJ_ | 8,010 | GVLDTPPSHIGLEHLPPPPEVPQFPLNLERLQALQDLVHRSLEAGYISPWDGPGNNPVFPVRKPNGAWRFVHDL |
| P03361_ | | RATNALTKPIPALSPGPPDLTAIPTHPPHIICLDLKDAFFQIPVEDRFRFYLSFTLPSPGGLQPHRRFAWRVLP |
| 2mut | | QGFINSPALFNRALQEPLRQVSAAFSQSLLVSYMDDILYASPTEEQRSQCYQALAARLRDLGFQVASEKTSQTP |
| | | SPVPFLGQMVHEQIVTYQSLPTLQISSPISLHQLQAVLGDLQWVSRGTPTTRRPLQLLYSSLKRHHDPRAIIQL |
| | | SPEQLQGIAELRQALSHNARSRYNEQEPLLAYVHLTRAGSTLVLFQKGAQFPLAYFQTPLTDNQASPWGLLLLL |
| | | GCQYLQTQALSSYAKPILKYYHNLPKTSLDNWIQSSEDPRVQELLQLWPQISSQGIQPPGPWKTLITRAEVFLT |
| | | PQFSPDPIPAALCLFSDGATGRGAYCLWKDHLLDFQAVPAPESAQKGELAGLLAGLAAAPPEPVNIWVDSKYLY |
| | | SLLRTWVLGAWLQPDPVPSYALLYKSLLRHPAIVVGHVRSHSSASHPIASLNNYVDQL |
| BLVJ_ | 8,011 | GVLDTPPSHIGLEHLPPPPEVPQFPLNLERLQALQDLVHRSLEAGYISPWDGPGNNPVFPVRKPNGAWRFVHDL |
| P03361_ | | RATNALTKPIPALSPGPPDLTAPPTHPPHIICLDLKDAFFQIPVEDRFRFYLSFTLPSPGGLQPHRRFAWRVLP |
| 2mutB | | QGFINSPALFQRALQEPLRQVSAAFSQSLLVSYMDDILYASPTEEQRSQCYQALAARLRDLGFQVASEKTSQTP |
| | | SPVPFLGQMVHEQIVTYQSLPTLQISSPISLHQLQAVLGDLQWVSRGTPTTRRPLQLLYSSLKRHHDPRAIIQL |
| | | SPEQLQGIAELRQALSHNARSRYNEQEPLLAYVHLTRAGSTLVLFQKGAQFPLAYFQTPLTDNQASPWGLLLLL |
| | | GCQYLQTQALSSYAKPILKYYHNLPKTSLDNWIQSSEDPRVQELLQLWPQISSQGIQPPGPWKTLITRAEVFLT |
| | | PQFSPDPIPAALCLFSDGATGRGAYCLWKDHLLDFQAVPAPESAQKGELAGLLAGLAAAPPEPVNIWVDSKYLY |
| | | SLLRTWVLGAWLQPDPVPSYALLYKSLLRHPAIVVGHVRSHSSASHPIASLNNYVDQL |
| FFV_ | 8,012 | MDLLKPLTVERKGVKIKGYWNSQADITCVPKDLLQGEEPVRQQNVTTIHGTQEGDVYYVNLKIDGRRINTEVIG |
| O93209 | | TTLDYAIITPGDVPWILKKPLELTIKLDLEEQQGTLLNNSILSKKGKEELKQLFEKYSALWQSWENQVGHRRIR |
| | | PHKIATGTVKPTPQKQYHINPKAKPDIQIVINDLLKQGVLIQKESTMNTPVYPVPKPNGRWRMVLDYRAVNKVT |
| | | PLIAVQNQHSYGILGSLFKGRYKTTIDLSNGFWAHPIVPEDYWITAFTWQGKQYCWTVLPQGFLNSPGLFTGDV |
| | | VDLLQGIPNVEVYVDDVYISHDSEKEHLEYLDILFNRLKEAGYIISLKKSNIANSIVDFLGFQITNEGRGLTDT |
| | | FKEKLENITAPTTLKQLQSILGLLNFARNFIPDFTELIAPLYALIPKSTKNYVPWQIEHSTTLETLITKLNGAE |
| | | YLQGRKGDKTLIMKVNASYTTGYIRYYNEGEKKPISYVSIVFSKTELKFTELEKLLTTVHKGLLKALDLSMGQN |
| | | IHVYSPIVSMQNIQKTPQTAKKALASRWLSWLSYLEDPRIRFFYDPQMPALKDLPAVDTGKDNKKHPSNFQHIF |
| | | YTDGSAITSPTKEGHLNAGMGIVYFINKDGNLQKQQEWSISLGNHTAQFAEIAAFEFALKKCLPLGGNILVVTD |
| | | SNYVAKAYNEELDVWASNGFVNNRKKPLKHISKWKSVADLKRLRPDVVVTHEPGHQKLDSSPHAYGNNLADQLA |
| | | TQASFKVH |
| FFV_ | 8,013 | MDLLKPLTVERKGVKIKGYWNSQADITCVPKDLLQGEEPVRQQNVTTIHGTQEGDVYYVNLKIDGRRINTEVIG |
| O93209 | | TTLDYAIITPGDVPWILKKPLELTIKLDLEEQQGTLLNNSILSKKGKEELKQLFEKYSALWQSWENQVGHRRIR |
| 2mut | | PHKIATGTVKPTPQKQYHINPKAKPDIQIVINDLLKQGVLIQKESTMNTPVYPVPKPNGRWRMVLDYRAVNKVT |
| | | PLIAVQNQHSYGILGSLFKGRYKTTIDLSNGFWAHPIVPEDYWITAFTWQGKQYCWTVLPQGFLNSPGLFNGDV |
| | | VDLLQGIPNVEVYVDDVYISHDSEKEHLEYLDILFNRLKEAGYIISLKKSNIANSIVDFLGFQITNEGRGLTDT |
| | | FKEKLENITAPTTLKQLQSILGLLNFARNFIPDFTELIAPLYALIPKSPKNYVPWQIEHSTTLETLITKLNGAE |
| | | YLQGRKGDKTLIMKVNASYTTGYIRYYNEGEKKPISYVSIVFSKTELKFTELEKLLTTVHKGLLKALDLSMGQN |
| | | IHVYSPIVSMQNIQKTPQTAKKALASRWLSWLSYLEDPRIRFFYDPQMPALKDLPAVDTGKDNKKHPSNFQHIF |
| | | YTDGSAITSPTKEGHLNAGMGIVYFINKDGNLQKQQEWSISLGNHTAQFAEIAAFEFALKKCLPLGGNILVVTD |
| | | SNYVAKAYNEELDVWASNGFVNNRKKPLKHISKWKSVADLKRLRPDVVVTHEPGHQKLDSSPHAYGNNLADQLA |
| | | TQASFKVH |
| FFV_ | 8,014 | MDLLKPLTVERKGVKIKGYWNSQADITCVPKDLLQGEEPVRQQNVTTIHGTQEGDVYYVNLKIDGRRINTEVIG |
| O93209 | | TTLDYAIITPGDVPWILKKPLELTIKLDLEEQQGTLLNNSILSKKGKEELKQLFEKYSALWQSWENQVGHRRIR |
| 2mutA | | PHKIATGTVKPTPQKQYHINPKAKPDIQIVINDLLKQGVLIQKESTMNTPVYPVPKPNGRWRMVLDYRAVNKVT |
| | | PLIAVQNQHSYGILGSLFKGRYKTTIDLSNGFWAHPIVPEDYWITAFTWQGKQYCWTVLPQGFLNSPGLFNGDV |
| | | VDLLQGIPNVEVYVDDVYISHDSEKEHLEYLDILFNRLKEAGYIISLKKSNIANSIVDFLGFQITNEGRGLTDT |
| | | FKEKLENITAPTTLKQLQSILGKLNFARNFIPDFTELIAPLYALIPKSPKNYVPWQIEHSTTLETLITKLNGAE |
| | | YLQGRKGDKTLIMKVNASYTTGYIRYYNEGEKKPISYVSIVFSKTELKFTELEKLLTTVHKGLLKALDLSMGQN |
| | | IHVYSPIVSMQNIQKTPQTAKKALASRWLSWLSYLEDPRIRFFYDPQMPALKDLPAVDTGKDNKKHPSNFQHIF |
| | | YTDGSAITSPTKEGHLNAGMGIVYFINKDGNLQKQQEWSISLGNHTAQFAEIAAFEFALKKCLPLGGNILVVTD |
| | | SNYVAKAYNEELDVWASNGFVNNRKKPLKHISKWKSVADLKRLRPDVVVTHEPGHQKLDSSPHAYGNNLADQLA |
| | | TQASFKVH |
| FFV_ | 8,015 | VPWILKKPLELTIKLDLEEQQGTLLNNSILSKKGKEELKQLFEKYSALWQSWENQVGHRRIRPHKIATGTVKPT |
| O93209- | | PQKQYHINPKAKPDIQIVINDLLKQGVLIQKESTMNTPVYPVPKPNGRWRMVLDYRAVNKVTPLIAVQNQHSYG |
| Pro | | ILGSLFKGRYKTTIDLSNGFWAHPIVPEDYWITAFTWQGKQYCWTVLPQGFLNSPGLFTGDVVDLLQGIPNVEV |
| | | YVDDVYISHDSEKEHLEYLDILFNRLKEAGYIISLKKSNIANSIVDFLGFQITNEGRGLTDTFKEKLENITAPT |
| | | TLKQLQSILGLLNFARNFIPDFTELIAPLYALIPKSTKNYVPWQIEHSTTLETLITKLNGAEYLQGRKGDKTLI |
| | | MKVNASYTTGYIRYYNEGEKKPISYVSIVFSKTELKFTELEKLLTTVHKGLLKALDLSMGQNIHVYSPIVSMQN |
| | | IQKTPQTAKKALASRWLSWLSYLEDPRIRFFYDPQMPALKDLPAVDTGKDNKKHPSNFQHIFYTDGSAITSPTK |
| | | EGHLNAGMGIVYFINKDGNLQKQQEWSISLGNHTAQFAEIAAFEFALKKCLPLGGNILVVTDSNYVAKAYNEEL |
| | | DVWASNGFVNNRKKPLKHISKWKSVADLKRLRPDVVVTHEPGHQKLDSSPHAYGNNLADQLATQASFKVH |
| FFV_ | 8,016 | VPWILKKPLELTIKLDLEEQQGTLLNNSILSKKGKEELKQLFEKYSALWQSWENQVGHRRIRPHKIATGTVKPT |
| O93209- | | PQKQYHINPKAKPDIQIVINDLLKQGVLIQKESTMNTPVYPVPKPNGRWRMVLDYRAVNKVTPLIAVQNQHSYG |
| Pro_ | | ILGSLFKGRYKTTIDLSNGFWAHPIVPEDYWITAFTWQGKQYCWTVLPQGFLNSPGLFNGDVVDLLQGIPNVEV |
| 2mut | | YVDDVYISHDSEKEHLEYLDILFNRLKEAGYIISLKKSNIANSIVDFLGFQITNEGRGLTDTFKEKLENITAPT |
| | | TLKQLQSILGLLNFARNFIPDFTELIAPLYALIPKSPKNYVPWQIEHSTTLETLITKLNGAEYLQGRKGDKTLI |
| | | MKVNASYTTGYIRYYNEGEKKPISYVSIVFSKTELKFTELEKLLTTVHKGLLKALDLSMGQNIHVYSPIVSMQN |
| | | IQKTPQTAKKALASRWLSWLSYLEDPRIRFFYDPQMPALKDLPAVDTGKDNKKHPSNFQHIFYTDGSAITSPTK |
| | | EGHLNAGMGIVYFINKDGNLQKQQEWSISLGNHTAQFAEIAAFEFALKKCLPLGGNILVVTDSNYVAKAYNEEL |
| | | DVWASNGFVNNRKKPLKHISKWKSVADLKRLRPDVVVTHEPGHQKLDSSPHAYGNNLADQLATQASFKVH |
| FFV_ | 8,017 | VPWILKKPLELTIKLDLEEQQGTLLNNSILSKKGKEELKQLFEKYSALWQSWENQVGHRRIRPHKIATGTVKPT |
| O93209- | | PQKQYHINPKAKPDIQIVINDLLKQGVLIQKESTMNTPVYPVPKPNGRWRMVLDYRAVNKVTPLIAVQNQHSYG |
| Pro_ | | ILGSLFKGRYKTTIDLSNGFWAHPIVPEDYWITAFTWQGKQYCWTVLPQGFLNSPGLFNGDVVDLLQGIPNVEV |
| 2mutA | | YVDDVYISHDSEKEHLEYLDILFNRLKEAGYIISLKKSNIANSIVDFLGFQITNEGRGLTDTFKEKLENITAPT |
| | | TLKQLQSILGKLNFARNFIPDFTELIAPLYALIPKSPKNYVPWQIEHSTTLETLITKLNGAEYLQGRKGDKTLI |
| | | MKVNASYTTGYIRYYNEGEKKPISYVSIVFSKTELKFTELEKLLTTVHKGLLKALDLSMGQNIHVYSPIVSMQN |
| | | IQKTPQTAKKALASRWLSWLSYLEDPRIRFFYDPQMPALKDLPAVDTGKDNKKHPSNFQHIFYTDGSAITSPTK |
| | | EGHLNAGMGIVYFINKDGNLQKQQEWSISLGNHTAQFAEIAAFEFALKKCLPLGGNILVVTDSNYVAKAYNEEL |
| | | DVWASNGFVNNRKKPLKHISKWKSVADLKRLRPDVVVTHEPGHQKLDSSPHAYGNNLADQLATQASFKVH |
| FLV_ | 8,018 | TLQLEEEYRLFEPESTQKQEMDIWLKNFPQAWAETGGMGTAHCQAPVLIQLKATATPISIRQYPMPHEAYQGIK |
| P10273 | | PHIRRMLDQGILKPCQSPWNTPLLPVKKPGTEDYRPVQDLREVNKRVEDIHPTVPNPYNLLSTLPPSHPWYTVL |
| | | DLKDAFFCLRLHSESQLLFAFEWRDPEIGLSGQLTWTRLPQGFKNSPTLFDEALHSDLADFRVRYPALVLLQYV |
| | | DDLLLAAATRTECLEGTKALLETLGNKGYRASAKKAQICLQEVTYLGYSLKDGQRWLTKARKEAILSIPVPKNS |
| | | RQVREFLGTAGYCRLWIPGFAELAAPLYPLTRPGTLFQWGTEQQLAFEDIKKALLSSPALGLPDITKPFELFID |
| | | ENSGFAKGVLVQKLGPWKRPVAYLSKKLDTVASGWPPCLRMVAAIAILVKDAGKLTLGQPLTILTSHPVEALVR |
| | | QPPNKWLSNARMTHYQAMLLDAERVHFGPTVSLNPATLLPLPSGGNHHDCLQILAETHGTRPDLTDQPLPDADL |
| | | TWYTDGSSFIRNGEREAGAAVTTESEVIWAAPLPPGTSAQRAELIALTQALKMAEGKKLTVYTDSRYAFATTHV |
| | | HGEIYRRRGLLTSEGKEIKNKNEILALLEALFLPKRLSIIHCPGHQKGDSPQAKGNRLADDTAKKAATETHSSL |
| | | TVLP |
| FLV_ | 8,019 | TLQLEEEYRLFEPESTQKQEMDIWLKNFPQAWAETGGMGTAHCQAPVLIQLKATATPISIRQYPMPHEAYQGIK |
| P10273 | | PHIRRMLDQGILKPCQSPWNTPLLPVKKPGTEDYRPVQDLREVNKRVEDIHPTVPNPYNLLSTLPPSHPWYTVL |
| 3mut | | DLKDAFFCLRLHSESQLLFAFEWRDPEIGLSGQLTWTRLPQGFKNSPTLFNEALHSDLADFRVRYPALVLLQYV |
| | | DDLLLAAATRTECLEGTKALLETLGNKGYRASAKKAQICLQEVTYLGYSLKDGQRWLTKARKEAILSIPVPKNS |
| | | RQVREFLGTAGYCRLWIPGFAELAAPLYPLTRPGTLFQWGTEQQLAFEDIKKALLSSPALGLPDITKPFELFID |
| | | ENSGFAKGVLVQKLGPWKRPVAYLSKKLDTVASGWPPCLRMVAAIAILVKDAGKLTLGQPLTILTSHPVEALVR |
| | | QPPNKWLSNARMTHYQAMLLDAERVHFGPTVSLNPATLLPLPSGGNHHDCLQILAETHGTRPDLTDQPLPDADL |
| | | TWYTDGSSFIRNGEREAGAAVTTESEVIWAAPLPPGTSAQRAELIALTQALKMAEGKKLTVYTDSRYAFATTHV |
| | | HGEIYRRRGWLTSEGKEIKNKNEILALLEALFLPKRLSIIHCPGHQKGDSPQAKGNRLADDTAKKAATETHSSL |
| | | TVLP |
| FLV_ | 8,020 | TLQLEEEYRLFEPESTQKQEMDIWLKNFPQAWAETGGMGTAHCQAPVLIQLKATATPISIRQYPMPHEAYQGIK |
| P10273_ | | PHIRRMLDQGILKPCQSPWNTPLLPVKKPGTEDYRPVQDLREVNKRVEDIHPTVPNPYNLLSTLPPSHPWYTVL |
| 3mutA | | DLKDAFFCLRLHSESQLLFAFEWRDPEIGLSGQLTWTRLPQGFKNSPTLFNEALHSDLADFRVRYPALVLLQYV |
| | | DDLLLAAATRTECLEGTKALLETLGNKGYRASAKKAQICLQEVTYLGYSLKDGQRWLTKARKEAILSIPVPKNS |
| | | RQVREFLGKAGYCRLFIPGFAELAAPLYPLTRPGTLFQWGTEQQLAFEDIKKALLSSPALGLPDITKPFELFID |
| | | ENSGFAKGVLVQKLGPWKRPVAYLSKKLDTVASGWPPCLRMVAAIAILVKDAGKLTLGQPLTILTSHPVEALVR |
| | | QPPNKWLSNARMTHYQAMLLDAERVHFGPTVSLNPATLLPLPSGGNHHDCLQILAETHGTRPDLTDQPLPDADL |
| | | TWYTDGSSFIRNGEREAGAAVTTESEVIWAAPLPPGTSAQRAELIALTQALKMAEGKKLTVYTDSRYAFATTHV |
| | | HGEIYRRRGWLTSEGKEIKNKNEILALLEALFLPKRLSIIHCPGHQKGDSPQAKGNRLADDTAKKAATETHSSL |
| | | TVLP |
| FOAMV_ | 8,021 | MNPLQLLQPLPAEIKGTKLLAHWNSGATITCIPESFLEDEQPIKKTLIKTIHGEKQQNVYYVTFKVKGRKVEAE |
| P14350 | | VIASPYEYILLSPTDVPWLTQQPLQLTILVPLQEYQEKILSKTALPEDQKQQLKTLFVKYDNLWQHWENQVGHR |
| | | KIRPHNIATGDYPPRPQKQYPINPKAKPSIQIVIDDLLKQGVLTPQNSTMNTPVYPVPKPDGRWRMVLDYREVN |
| | | KTIPLTAAQNQHSAGILATIVRQKYKTTLDLANGFWAHPITPESYWLTAFTWQGKQYCWTRLPQGFLNSPALFT |
| | | ADVVDLLKEIPNVQVYVDDIYLSHDDPKEHVQQLEKVFQILLQAGYVVSLKKSEIGQKTVEFLGFNITKEGRGL |
| | | TDTFKTKLLNITPPKDLKQLQSILGLLNFARNFIPNFAELVQPLYNLIASAKGKYIEWSEENTKQLNMVIEALN |
| | | TASNLEERLPEQRLVIKVNTSPSAGYVRYYNETGKKPIMYLNYVFSKAELKFSMLEKLLTTMHKALIKAMDLAM |
| | | GQEILVYSPIVSMTKIQKTPLPERKALPIRWITWMTYLEDPRIQFHYDKTLPELKHIPDVYTSSQSPVKHPSQY |
| | | EGVFYTDGSAIKSPDPTKSNNAGMGIVHATYKPEYQVLNQWSIPLGNHTAQMAEIAAVEFACKKALKIPGPVLV |
| | | ITDSFYVAESANKELPYWKSNGFVNNKKKPLKHISKWKSIAECLSMKPDITIQHEKGISLQIPVFILKGNALAD |
| | | KLATQGSYVVN |
| FOAMV_ | 8,022 | MNPLQLLQPLPAEIKGTKLLAHWNSGATITCIPESFLEDEQPIKKTLIKTIHGEKQQNVYYVTFKVKGRKVEAE |
| P14350_ | | VIASPYEYILLSPTDVPWLTQQPLQLTILVPLQEYQEKILSKTALPEDQKQQLKTLFVKYDNLWQHWENQVGHR |
| 2mut | | KIRPHNIATGDYPPRPQKQYPINPKAKPSIQIVIDDLLKQGVLTPQNSTMNTPVYPVPKPDGRWRMVLDYREVN |
| | | KTIPLTAAQNQHSAGILATIVRQKYKTTLDLANGFWAHPITPESYWLTAFTWQGKQYCWTRLPQGFLNSPALFN |
| | | ADVVDLLKEIPNVQVYVDDIYLSHDDPKEHVQQLEKVFQILLQAGYVVSLKKSEIGQKTVEFLGFNITKEGRGL |
| | | TDTFKTKLLNITPPKDLKQLQSILGLLNFARNFIPNFAELVQPLYNLIAPAKGKYIEWSEENTKQLNMVIEALN |
| | | TASNLEERLPEQRLVIKVNTSPSAGYVRYYNETGKKPIMYLNYVFSKAELKFSMLEKLLTTMHKALIKAMDLAM |
| | | GQEILVYSPIVSMTKIQKTPLPERKALPIRWITWMTYLEDPRIQFHYDKTLPELKHIPDVYTSSQSPVKHPSQY |
| | | EGVFYTDGSAIKSPDPTKSNNAGMGIVHATYKPEYQVLNQWSIPLGNHTAQMAEIAAVEFACKKALKIPGPVLV |
| | | ITDSFYVAESANKELPYWKSNGFVNNKKKPLKHISKWKSIAECLSMKPDITIQHEKGISLQIPVFILKGNALAD |
| | | KLATQGSYVVN |
| FOAMV_ | 8,023 | MNPLQLLQPLPAEIKGTKLLAHWNSGATITCIPESFLEDEQPIKKTLIKTIHGEKQQNVYYVTFKVKGRKVEAE |
| P14350_ | | VIASPYEYILLSPTDVPWLTQQPLQLTILVPLQEYQEKILSKTALPEDQKQQLKTLFVKYDNLWQHWENQVGHR |
| 2mutA | | KIRPHNIATGDYPPRPQKQYPINPKAKPSIQIVIDDLLKQGVLTPQNSTMNTPVYPVPKPDGRWRMVLDYREVN |
| | | KTIPLTAAQNQHSAGILATIVRQKYKTTLDLANGFWAHPITPESYWLTAFTWQGKQYCWTRLPQGFLNSPALFN |
| | | ADVVDLLKEIPNVQVYVDDIYLSHDDPKEHVQQLEKVFQILLQAGYVVSLKKSEIGQKTVEFLGFNITKEGRGL |
| | | TDTFKTKLLNITPPKDLKQLQSILGKLNFARNFIPNFAELVQPLYNLIAPAKGKYIEWSEENTKQLNMVIEALN |
| | | TASNLEERLPEQRLVIKVNTSPSAGYVRYYNETGKKPIMYLNYVFSKAELKFSMLEKLLTTMHKALIKAMDLAM |
| | | GQEILVYSPIVSMTKIQKTPLPERKALPIRWITWMTYLEDPRIQFHYDKTLPELKHIPDVYTSSQSPVKHPSQY |
| | | EGVFYTDGSAIKSPDPTKSNNAGMGIVHATYKPEYQVLNQWSIPLGNHTAQMAEIAAVEFACKKALKIPGPVLV |
| | | ITDSFYVAESANKELPYWKSNGFVNNKKKPLKHISKWKSIAECLSMKPDITIQHEKGISLQIPVFILKGNALAD |
| | | KLATQGSYVVN |
| FOAMV_ | 8,024 | VPWLTQQPLQLTILVPLQEYQEKILSKTALPEDQKQQLKTLFVKYDNLWQHWENQVGHRKIRPHNIATGDYPPR |
| P14350- | | PQKQYPINPKAKPSIQIVIDDLLKQGVLTPQNSTMNTPVYPVPKPDGRWRMVLDYREVNKTIPLTAAQNQHSAG |
| Pro | | ILATIVRQKYKTTLDLANGFWAHPITPESYWLTAFTWQGKQYCWTRLPQGFLNSPALFTADVVDLLKEIPNVQV |
| | | YVDDIYLSHDDPKEHVQQLEKVFQILLQAGYVVSLKKSEIGQKTVEFLGFNITKEGRGLTDTFKTKLLNITPPK |
| | | DLKQLQSILGLLNFARNFIPNFAELVQPLYNLIASAKGKYIEWSEENTKQLNMVIEALNTASNLEERLPEQRLV |
| | | IKVNTSPSAGYVRYYNETGKKPIMYLNYVFSKAELKFSMLEKLLTTMHKALIKAMDLAMGQEILVYSPIVSMTK |
| | | IQKTPLPERKALPIRWITWMTYLEDPRIQFHYDKTLPELKHIPDVYTSSQSPVKHPSQYEGVFYTDGSAIKSPD |
| | | PTKSNNAGMGIVHATYKPEYQVLNQWSIPLGNHTAQMAEIAAVEFACKKALKIPGPVLVITDSFYVAESANKEL |
| | | PYWKSNGFVNNKKKPLKHISKWKSIAECLSMKPDITIQHEKGISLQIPVFILKGNALADKLATQGSYVVN |
| FOAMV_ | 8,025 | VPWLTQQPLQLTILVPLQEYQEKILSKTALPEDQKQQLKTLFVKYDNLWQHWENQVGHRKIRPHNIATGDYPPR |
| P14350- | | PQKQYPINPKAKPSIQIVIDDLLKQGVLTPQNSTMNTPVYPVPKPDGRWRMVLDYREVNKTIPLTAAQNQHSAG |
| Pro_ | | ILATIVRQKYKTTLDLANGFWAHPITPESYWLTAFTWQGKQYCWTRLPQGFLNSPALFNADVVDLLKEIPNVQV |
| 2mut | | YVDDIYLSHDDPKEHVQQLEKVFQILLQAGYVVSLKKSEIGQKTVEFLGFNITKEGRGLTDTFKTKLLNITPPK |
| | | DLKQLQSILGLLNFARNFIPNFAELVQPLYNLIAPAKGKYIEWSEENTKQLNMVIEALNTASNLEERLPEQRLV |
| | | IKVNTSPSAGYVRYYNETGKKPIMYLNYVFSKAELKFSMLEKLLTTMHKALIKAMDLAMGQEILVYSPIVSMTK |
| | | IQKTPLPERKALPIRWITWMTYLEDPRIQFHYDKTLPELKHIPDVYTSSQSPVKHPSQYEGVFYTDGSAIKSPD |
| | | PTKSNNAGMGIVHATYKPEYQVLNQWSIPLGNHTAQMAEIAAVEFACKKALKIPGPVLVITDSFYVAESANKEL |
| | | PYWKSNGFVNNKKKPLKHISKWKSIAECLSMKPDITIQHEKGISLQIPVFILKGNALADKLATQGSYVVN |
| FOAMV_ | 8,026 | VPWLTQQPLQLTILVPLQEYQEKILSKTALPEDQKQQLKTLFVKYDNLWQHWENQVGHRKIRPHNIATGDYPPR |
| P14350- | | PQKQYPINPKAKPSIQIVIDDLLKQGVLTPQNSTMNTPVYPVPKPDGRWRMVLDYREVNKTIPLTAAQNQHSAG |
| Pro_ | | ILATIVRQKYKTTLDLANGFWAHPITPESYWLTAFTWQGKQYCWTRLPQGFLNSPALFNADVVDLLKEIPNVQV |
| 2mutA | | YVDDIYLSHDDPKEHVQQLEKVFQILLQAGYVVSLKKSEIGQKTVEFLGFNITKEGRGLTDTFKTKLLNITPPK |
| | | DLKQLQSILGKLNFARNFIPNFAELVQPLYNLIAPAKGKYIEWSEENTKQLNMVIEALNTASNLEERLPEQRLV |
| | | IKVNTSPSAGYVRYYNETGKKPIMYLNYVFSKAELKFSMLEKLLTTMHKALIKAMDLAMGQEILVYSPIVSMTK |
| | | IQKTPLPERKALPIRWITWMTYLEDPRIQFHYDKTLPELKHIPDVYTSSQSPVKHPSQYEGVFYTDGSAIKSPD |
| | | PTKSNNAGMGIVHATYKPEYQVLNQWSIPLGNHTAQMAEIAAVEFACKKALKIPGPVLVITDSFYVAESANKEL |
| | | PYWKSNGFVNNKKKPLKHISKWKSIAECLSMKPDITIQHEKGISLQIPVFILKGNALADKLATQGSYVVN |
| GALV_ | 8,027 | VLNLEEEYRLHEKPVPSSIDPSWLQLFPTVWAERAGMGLANQVPPVVVELRSGASPVAVRQYPMSKEAREGIRP |
| P21414 | | HIQKFLDLGVLVPCRSPWNTPLLPVKKPGTNDYRPVQDLREINKRVQDIHPTVPNPYNLLSSLPPSYTWYSVLD |
| | | LKDAFFCLRLHPNSQPLFAFEWKDPEKGNTGQLTWTRLPQGFKNSPTLFDEALHRDLAPFRALNPQVVLLQYVD |
| | | DLLVAAPTYEDCKKGTQKLLQELSKLGYRVSAKKAQLCQREVTYLGYLLKEGKRWLTPARKATVMKIPVPTTPR |
| | | QVREFLGTAGFCRLWIPGFASLAAPLYPLTKESIPFIWTEEHQQAFDHIKKALLSAPALALPDLTKPFTLYIDE |
| | | RAGVARGVLTQTLGPWRRPVAYLSKKLDPVASGWPTCLKAVAAVALLLKDADKLTLGQNVTVIASHSLESIVRQ |
| | | PPDRWMTNARMTHYQSLLLNERVSFAPPAVLNPATLLPVESEATPVHRCSEILAEETGTRRDLEDQPLPGVPTW |
| | | YTDGSSFITEGKRRAGAPIVDGKRTVWASSLPEGTSAQKAELVALTQALRLAEGKNINIYTDSRYAFATAHIHG |
| | | AIYKQRGLLTSAGKDIKNKEEILALLEAIHLPRRVAIIHCPGHQRGSNPVATGNRRADEAAKQAALSTRVLAGT |
| | | TKP |
| GALV_ | 8,028 | VLNLEEEYRLHEKPVPSSIDPSWLQLFPTVWAERAGMGLANQVPPVVVELRSGASPVAVRQYPMSKEAREGIRP |
| P21414_ | | HIQKFLDLGVLVPCRSPWNTPLLPVKKPGTNDYRPVQDLREINKRVQDIHPTVPNPYNLLSSLPPSYTWYSVLD |
| 3mut | | LKDAFFCLRLHPNSQPLFAFEWKDPEKGNTGQLTWTRLPQGFKNSPTLFNEALHRDLAPFRALNPQVVLLQYVD |
| | | DLLVAAPTYEDCKKGTQKLLQELSKLGYRVSAKKAQLCQREVTYLGYLLKEGKRWLTPARKATVMKIPVPTTPR |
| | | QVREFLGTAGFCRLWIPGFASLAAPLYPLTKPSIPFIWTEEHQQAFDHIKKALLSAPALALPDLTKPFTLYIDE |
| | | RAGVARGVLTQTLGPWRRPVAYLSKKLDPVASGWPTCLKAVAAVALLLKDADKLTLGQNVTVIASHSLESIVRQ |
| | | PPDRWMTNARMTHYQSLLLNERVSFAPPAVLNPATLLPVESEATPVHRCSEILAEETGTRRDLEDQPLPGVPTW |
| | | YTDGSSFITEGKRRAGAPIVDGKRTVWASSLPEGTSAQKAELVALTQALRLAEGKNINIYTDSRYAFATAHIHG |
| | | AIYKQRGWLTSAGKDIKNKEEILALLEAIHLPRRVAIIHCPGHQRGSNPVATGNRRADEAAKQAALSTRVLAGT |
| | | TKP |
| GALV_ | 8,029 | VLNLEEEYRLHEKPVPSSIDPSWLQLFPTVWAERAGMGLANQVPPVVVELRSGASPVAVRQYPMSKEAREGIRP |
| P21414_ | | HIQKFLDLGVLVPCRSPWNTPLLPVKKPGTNDYRPVQDLREINKRVQDIHPTVPNPYNLLSSLPPSYTWYSVLD |
| 3mutA | | LKDAFFCLRLHPNSQPLFAFEWKDPEKGNTGQLTWTRLPQGFKNSPTLFNEALHRDLAPFRALNPQVVLLQYVD |
| | | DLLVAAPTYEDCKKGTQKLLQELSKLGYRVSAKKAQLCQREVTYLGYLLKEGKRWLTPARKATVMKIPVPTTPR |
| | | QVREFLGKAGFCRLFIPGFASLAAPLYPLTKPSIPFIWTEEHQQAFDHIKKALLSAPALALPDLTKPFTLYIDE |
| | | RAGVARGVLTQTLGPWRRPVAYLSKKLDPVASGWPTCLKAVAAVALLLKDADKLTLGQNVTVIASHSLESIVRQ |
| | | PPDRWMTNARMTHYQSLLLNERVSFAPPAVLNPATLLPVESEATPVHRCSEILAEETGTRRDLEDQPLPGVPTW |
| | | YTDGSSFITEGKRRAGAPIVDGKRTVWASSLPEGTSAQKAELVALTQALRLAEGKNINIYTDSRYAFATAHIHG |
| | | AIYKQRGWLTSAGKDIKNKEEILALLEAIHLPRRVAIIHCPGHQRGSNPVATGNRRADEAAKQAALSTRVLAGT |
| | | TKP |
| HTL1A_ | 8,030 | AVLGLEHLPRPPQISQFPLNPERLQALQHLVRKALEAGHIEPYTGPGNNPVFPVKKANGTWRFIHDLRATNSLT |
| P03362 | | IDLSSSSPGPPDLSSLPTTLAHLQTIDLRDAFFQIPLPKQFQPYFAFTVPQQCNYGPGTRYAWKVLPQGFKNSP |
| | | TLFEMQLAHILQPIRQAFPQCTILQYMDDILLASPSHEDLLLLSEATMASLISHGLPVSENKTQQTPGTIKFLG |
| | | QIISPNHLTYDAVPTVPIRSRWALPELQALLGEIQWVSKGTPTLRQPLHSLYCALQRHTDPRDQIYLNPSQVQS |
| | | LVQLRQALSQNCRSRLVQTLPLLGAIMLTLTGTTTVVFQSKEQWPLVWLHAPLPHTSQCPWGQLLASAVLLLDK |
| | | YTLQSYGLLCQTIHHNISTQTFNQFIQTSDHPSVPILLHHSHRFKNLGAQTGELWNTFLKTAAPLAPVKALMPV |
| | | FTLSPVIINTAPCLFSDGSTSRAAYILWDKQILSQRSFPLPPPHKSAQRAELLGLLHGLSSARSWRCLNIFLDS |
| | | KYLYHYLRTLALGTFQGRSSQAPFQALLPRLLSRKVVYLHHVRSHTNLPDPISRLNALTDALLITPVLQL |
| HTL1A_ | 8,031 | AVLGLEHLPRPPQISQFPLNPERLQALQHLVRKALEAGHIEPYTGPGNNPVFPVKKANGTWRFIHDLRATNSLT |
| P03362_ | | IDLSSSSPGPPDLSSLPTTLAHLQTIDLRDAFFQIPLPKQFQPYFAFTVPQQCNYGPGTRYAWKVLPQGFKNSP |
| 2mut | | TLFQMQLAHILQPIRQAFPQCTILQYMDDILLASPSHEDLLLLSEATMASLISHGLPVSENKTQQTPGTIKFLG |
| | | QIISPNHLTYDAVPTVPIRSRWALPELQALLGEIQWVSKGTPTLRQPLHSLYCALQPHTDPRDQIYLNPSQVQS |
| | | LVQLRQALSQNCRSRLVQTLPLLGAIMLTLTGTTTVVFQSKEQWPLVWLHAPLPHTSQCPWGQLLASAVLLLDK |
| | | YTLQSYGLLCQTIHHNISTQTFNQFIQTSDHPSVPILLHHSHRFKNLGAQTGELWNTFLKTAAPLAPVKALMPV |
| | | FTLSPVIINTAPCLFSDGSTSRAAYILWDKQILSQRSFPLPPPHKSAQRAELLGLLHGLSSARSWRCLNIFLDS |
| | | KYLYHYLRTLALGTFQGRSSQAPFQALLPRLLSRKVVYLHHVRSHTNLPDPISRLNALTDALLITPVLQL |
| HTL1A_ | 8,032 | AVLGLEHLPRPPQISQFPLNPERLQALQHLVRKALEAGHIEPYTGPGNNPVFPVKKANGTWRFIHDLRATNSLT |
| P03362_ | | IDLSSSSPGPPDLSSPPTTLAHLQTIDLRDAFFQIPLPKQFQPYFAFTVPQQCNYGPGTRYAWKVLPQGFKNSP |
| 2mutB | | TLFQMQLAHILQPIRQAFPQCTILQYMDDILLASPSHEDLLLLSEATMASLISHGLPVSENKTQQTPGTIKFLG |
| | | QIISPNHLTYDAVPTVPIRSRWALPELQALLGEIQWVSKGTPTLRQPLHSLYCALQPHTDPRDQIYLNPSQVQS |
| | | LVQLRQALSQNCRSRLVQTLPLLGAIMLTLTGTTTVVFQSKEQWPLVWLHAPLPHTSQCPWGQLLASAVLLLDK |
| | | YTLQSYGLLCQTIHHNISTQTFNQFIQTSDHPSVPILLHHSHRFKNLGAQTGELWNTFLKTAAPLAPVKALMPV |
| | | FTLSPVIINTAPCLFSDGSTSRAAYILWDKQILSQRSFPLPPPHKSAQRAELLGLLHGLSSARSWRCLNIFLDS |
| | | KYLYHYLRTLALGTFQGRSSQAPFQALLPRLLSRKVVYLHHVRSHTNLPDPISRLNALTDALLITPVLQL |
| HTL1C_ | 8,033 | AVLGLEHLPRPPEISQFPLNPERLQALQHLVRKALEAGHIEPYTGPGNNPVFPVKKANGTWRFIHDLRATNSLT |
| P14078 | | IDLSSSSPGPPDLSSLPTTLAHLQTIDLKDAFFQIPLPKQFQPYFAFTVPQQCNYGPGTRYAWRVLPQGFKNSP |
| | | TLFEMQLAHILQPIRQAFPQCTILQYMDDILLASPSHADLQLLSEATMASLISHGLPVSENKTQQTPGTIKFLG |
| | | QIISPNHLTYDAVPKVPIRSRWALPELQALLGEIQWVSKGTPTLRQPLHSLYCALQRHTDPRDQIYLNPSQVQS |
| | | LVQLRQALSQNCRSRLVQTLPLLGAIMLTLTGTTTVVFQSKQQWPLVWLHAPLPHTSQCPWGQLLASAVLLLDK |
| | | YTLQSYGLLCQTIHHNISTQTFNQFIQTSDHPSVPILLHHSHRFKNLGAQTGELWNTFLKTTAPLAPVKALMPV |
| | | FTLSPVIINTAPCLFSDGSTSQAAYILWDKHILSQRSFPLPPPHKSAQRAELLGLLHGLSSARSWRCLNIFLDS |
| | | KYLYHYLRTLALGTFQGRSSQAPFQALLPRLLSRKVVYLHHVRSHTNLPDPISRLNALTDALLITPVLQL |
| HTL1C | 8,034 | AVLGLEHLPRPPEISQFPLNPERLQALQHLVRKALEAGHIEPYTGPGNNPVFPVKKANGTWRFIHDLRATNSLT |
| P14078_ | | IDLSSSSPGPPDLSSLPTTLAHLQTIDLKDAFFQIPLPKQFQPYFAFTVPQQCNYGPGTRYAWRVLPQGFKNSP |
| 2mut | | TLFQMQLAHILQPIRQAFPQCTILQYMDDILLASPSHADLQLLSEATMASLISHGLPVSENKTQQTPGTIKFLG |
| | | QIISPNHLTYDAVPKVPIRSRWALPELQALLGEIQWVSKGTPTLRQPLHSLYCALQPHTDPRDQIYLNPSQVQS |
| | | LVQLRQALSQNCRSRLVQTLPLLGAIMLTLTGTTTVVFQSKQQWPLVWLHAPLPHTSQCPWGQLLASAVLLLDK |
| | | YTLQSYGLLCQTIHHNISTQTFNQFIQTSDHPSVPILLHHSHRFKNLGAQTGELWNTFLKTTAPLAPVKALMPV |
| | | FTLSPVIINTAPCLFSDGSTSQAAYILWDKHILSQRSFPLPPPHKSAQRAELLGLLHGLSSARSWRCLNIFLDS |
| | | KYLYHYLRTLALGTFQGRSSQAPFQALLPRLLSRKVVYLHHVRSHTNLPDPISRLNALTDALLITPVLQL |
| HTL1L_ | 8,035 | GLEHLPRPPEISQFPLNPERLQALQHLVRKALEAGHIEPYTGPGNNPVFPVKKANGTWRFIHDLRATNSLTVDL |
| P0C211 | | SSSSPGPPDLSSLPTTLAHLQTIDLKDAFFQIPLPKQFQPYFAFTVPQQCNYGPGTRYAWKVLPQGFKNSPTLF |
| | | EMQLASILQPIRQAFPQCVILQYMDDILLASPSPEDLQQLSEATMASLISHGLPVSQDKTQQTPGTIKFLGQII |
| | | SPNHITYDAVPTVPIRSRWALPELQALLGEIQWVSKGTPTLRQPLHSLYCALQGHTDPRDQIYLNPSQVQSLMQ |
| | | LQQALSQNCRSRLAQTLPLLGAIMLTLTGTTTVVFQSKQQWPLVWLHAPLPHTSQCPWGQLLASAVLLLDKYTL |
| | | QSYGLLCQTIHHNISIQTFNQFIQTSDHPSVPILLHHSHRFKNLGAQTGELWNTFLKTAAPLAPVKALTPVFTL |
| | | SPIIINTAPCLFSDGSTSQAAYILWDKHILSQRSFPLPPPHKSAQQAELLGLLHGLSSARSWHCLNIFLDSKYL |
| | | YHYLRTLALGTFQGKSSQAPFQALLPRLLAHKVIYLHHVRSHTNLPDPISKLNALTDALLITPIL |
| HTL1L_ | 8,036 | GLEHLPRPPEISQFPLNPERLQALQHLVRKALEAGHIEPYTGPGNNPVFPVKKANGTWRFIHDLRATNSLTVDL |
| P0C211_ | | SSSSPGPPDLSSLPTTLAHLQTIDLKDAFFQIPLPKQFQPYFAFTVPQQCNYGPGTRYAWKVLPQGFKNSPTLF |
| 2mut | | QMQLASILQPIRQAFPQCVILQYMDDILLASPSPEDLQQLSEATMASLISHGLPVSQDKTQQTPGTIKFLGQII |
| | | SPNHITYDAVPTVPIRSRWALPELQALLGEIQWVSKGTPTLRQPLHSLYCALQGHTDPRDQIYLNPSQVQSLMQ |
| | | LQQALSQNCRSRLAQTLPLLGAIMLTLTGTTTVVFQSKQQWPLVWLHAPLPHTSQCPWGQLLASAVLLLDKYTL |
| | | QSYGLLCQTIHHNISIQTFNQFIQTSDHPSVPILLHHSHRFKNLGAQTGELWNTFLKTAAPLAPVKALTPVFTL |
| | | SPIIINTAPCLFSDGSTSQAAYILWDKHILSQRSFPLPPPHKSAQQAELLGLLHGLSSARSWHCLNIFLDSKYL |
| | | YHYLRTLAWGTFQGKSSQAPFQALLPRLLAHKVIYLHHVRSHTNLPDPISKLNALTDALLITPIL |
| HTL1L_ | 8,037 | GLEHLPRPPEISQFPLNPERLQALQHLVRKALEAGHIEPYTGPGNNPVFPVKKANGTWRFIHDLRATNSLTVDL |
| P0C211_ | | SSSSPGPPDLSSPPTTLAHLQTIDLKDAFFQIPLPKQFQPYFAFTVPQQCNYGPGTRYAWKVLPQGFKNSPTLF |
| 2mutB | | QMQLASILQPIRQAFPQCVILQYMDDILLASPSPEDLQQLSEATMASLISHGLPVSQDKTQQTPGTIKFLGQII |
| | | SPNHITYDAVPTVPIRSRWALPELQALLGEIQWVSKGTPTLRQPLHSLYCALQGHTDPRDQIYLNPSQVQSLMQ |
| | | LQQALSQNCRSRLAQTLPLLGAIMLTLTGTTTVVFQSKQQWPLVWLHAPLPHTSQCPWGQLLASAVLLLDKYTL |
| | | QSYGLLCQTIHHNISIQTFNQFIQTSDHPSVPILLHHSHRFKNLGAQTGELWNTFLKTAAPLAPVKALTPVFTL |
| | | SPIIINTAPCLFSDGSTSQAAYILWDKHILSQRSFPLPPPHKSAQQAELLGLLHGLSSARSWHCLNIFLDSKYL |
| | | YHYLRTLAWGTFQGKSSQAPFQALLPRLLAHKVIYLHHVRSHTNLPDPISKLNALTDALLITPIL |
| HTL32_ | 8,038 | GLEHLPPPPEVSQFPLNPERLQALTDLVSRALEAKHIEPYQGPGNNPIFPVKKPNGKWRFIHDLRATNSVTRDL |
| Q0R5R2 | | ASPSPGPPDLTSLPQGLPHLRTIDLTDAFFQIPLPTIFQPYFAFTLPQPNNYGPGTRYSWRVLPQGFKNSPTLF |
| | | EQQLSHILTPVRKTFPNSLIIQYMDDILLASPAPGELAALTDKVTNALTKEGLPLSPEKTQATPGPIHFLGQVI |
| | | SQDCITYETLPSINVKSTWSLAELQSMLGELQWVSKGTPVLRSSLHQLYLALRGHRDPRDTIKLTSIQVQALRT |
| | | IQKALTLNCRSRLVNQLPILALIMLRPTGTTAVLFQTKQKWPLVWLHTPHPATSLRPWGQLLANAVIILDKYSL |
| | | QHYGQVCKSFHHNISNQALTYYLHTSDQSSVAILLQHSHRFHNLGAQPSGPWRSLLQMPQIFQNIDVLRPPFTI |
| | | SPVVINHAPCLFSDGSASKAAFIIWDRQVIHQQVLSLPSTCSAQAGELFGLLAGLQKSQPWVALNIFLDSKFLI |
| | | GHLRRMALGAFPGPSTQCELHTQLLPLLQGKTVYVHHVRSHTLLQDPISRLNEATDALMLAPLLPL |
| HTL32_ | 8,039 | GLEHLPPPPEVSQFPLNPERLQALTDLVSRALEAKHIEPYQGPGNNPIFPVKKPNGKWRFIHDLRATNSVTRDL |
| QOR5R2_ | | ASPSPGPPDLTSLPQGLPHLRTIDLTDAFFQIPLPTIFQPYFAFTLPQPNNYGPGTRYSWRVLPQGFKNSPTLF |
| 2mut | | QQQLSHILTPVRKTFPNSLIIQYMDDILLASPAPGELAALTDKVTNALTKEGLPLSPEKTQATPGPIHFLGQVI |
| | | SQDCITYETLPSINVKSTWSLAELQSMLGELQWVSKGTPVLRSSLHQLYLALRGHRDPRDTIKLTSIQVQALRT |
| | | IQKALTLNCRSRLVNQLPILALIMLRPTGTTAVLFQTKQKWPLVWLHTPHPATSLRPWGQLLANAVIILDKYSL |
| | | QHYGQVCKSFHHNISNQALTYYLHTSDQSSVAILLQHSHRFHNLGAQPSGPWRSLLQMPQIFQNIDVLRPPFTI |
| | | SPVVINHAPCLFSDGSASKAAFIIWDRQVIHQQVLSLPSTCSAQAGELFGLLAGLQKSQPWVALNIFLDSKFLI |
| | | GHLRRMAWGAFPGPSTQCELHTQLLPLLQGKTVYVHHVRSHTLLQDPISRLNEATDALMLAPLLPL |
| HTL32_ | 8,040 | GLEHLPPPPEVSQFPLNPERLQALTDLVSRALEAKHIEPYQGPGNNPIFPVKKPNGKWRFIHDLRATNSVTRDL |
| QOR5R2_ | | ASPSPGPPDLTSPPQGLPHLRTIDLTDAFFQIPLPTIFQPYFAFTLPQPNNYGPGTRYSWRVLPQGFKNSPTLF |
| 2mutB | | QQQLSHILTPVRKTFPNSLIIQYMDDILLASPAPGELAALTDKVTNALTKEGLPLSPEKTQATPGPIHFLGQVI |
| | | SQDCITYETLPSINVKSTWSLAELQSMLGELQWVSKGTPVLRSSLHQLYLALRGHRDPRDTIKLTSIQVQALRT |
| | | IQKALTLNCRSRLVNQLPILALIMLRPTGTTAVLFQTKQKWPLVWLHTPHPATSLRPWGQLLANAVIILDKYSL |
| | | QHYGQVCKSFHHNISNQALTYYLHTSDQSSVAILLQHSHRFHNLGAQPSGPWRSLLQMPQIFQNIDVLRPPFTI |
| | | SPVVINHAPCLFSDGSASKAAFIIWDRQVIHQQVLSLPSTCSAQAGELFGLLAGLQKSQPWVALNIFLDSKFLI |
| | | GHLRRMAWGAFPGPSTQCELHTQLLPLLQGKTVYVHHVRSHTLLQDPISRLNEATDALMLAPLLPL |
| HTL3P_ | 8,041 | GLEHLPPPPEVSQFPLNPERLQALTDLVSRALEAKHIEPYQGPGNNPIFPVKKPNGKWRFIHDLRATNSLTRDL |
| Q400X6 | | ASPSPGPPDLTSLPQDLPHLRTIDLTDAFFQIPLPAVFQPYFAFTLPQPNNHGPGTRYSWRVLPQGFKNSPTLF |
| | | EQQLSHILAPVRKAFPNSLIIQYMDDILLASPALRELTALTDKVTNALTKEGLPMSLEKTQATPGSIHFLGQVI |
| | | SPDCITYETLPSIHVKSIWSLAELQSMLGELQWVSKGTPVLRSSLHQLYLALRGHRDPRDTIELTSTQVQALKT |
| | | IQKALALNCRSRLVSQLPILALIILRPTGTTAVLFQTKQKWPLVWLHTPHPATSLRPWGQLLANAIITLDKYSL |
| | | QHYGQICKSFHHNISNQALTYYLHTSDQSSVAILLQHSHRFHNLGAQPSGPWRSLLQVPQIFQNIDVLRPPFII |
| | | SPVVIDHAPCLFSDGATSKAAFILWDKQVIHQQVLPLPSTCSAQAGELFGLLAGLQKSKPWPALNIFLDSKFLI |
| | | GHLRRMALGAFLGPSTQCDLHARLFPLLQGKTVYVHHVRSHTLLQDPISRLNEATDALMLAPLLPL |
| HTL3P_ | 8,042 | GLEHLPPPPEVSQFPLNPERLQALTDLVSRALEAKHIEPYQGPGNNPIFPVKKPNGKWRFIHDLRATNSLTRDL |
| Q4U0X6_ | | ASPSPGPPDLTSLPQDLPHLRTIDLTDAFFQIPLPAVFQPYFAFTLPQPNNHGPGTRYSWRVLPQGFKNSPTLF |
| 2mut | | QQQLSHILAPVRKAFPNSLIIQYMDDILLASPALRELTALTDKVTNALTKEGLPMSLEKTQATPGSIHFLGQVI |
| | | SPDCITYETLPSIHVKSIWSLAELQSMLGELQWVSKGTPVLRSSLHQLYLALRGHRDPRDTIELTSTQVQALKT |
| | | IQKALALNCRSRLVSQLPILALIILRPTGTTAVLFQTKQKWPLVWLHTPHPATSLRPWGQLLANAIITLDKYSL |
| | | QHYGQICKSFHHNISNQALTYYLHTSDQSSVAILLQHSHRFHNLGAQPSGPWRSLLQVPQIFQNIDVLRPPFII |
| | | SPVVIDHAPCLFSDGATSKAAFILWDKQVIHQQVLPLPSTCSAQAGELFGLLAGLQKSKPWPALNIFLDSKFLI |
| | | GHLRRMAWGAFLGPSTQCDLHARLFPLLQGKTVYVHHVRSHTLLQDPISRLNEATDALMLAPLLPL |
| HTL3P_ | 8,043 | GLEHLPPPPEVSQFPLNPERLQALTDLVSRALEAKHIEPYQGPGNNPIFPVKKPNGKWRFIHDLRATNSLTRDL |
| Q400X6_ | | ASPSPGPPDLTSPPQDLPHLRTIDLTDAFFQIPLPAVFQPYFAFTLPQPNNHGPGTRYSWRVLPQGFKNSPTLF |
| 2mutB | | QQQLSHILAPVRKAFPNSLIIQYMDDILLASPALRELTALTDKVTNALTKEGLPMSLEKTQATPGSIHFLGQVI |
| | | SPDCITYETLPSIHVKSIWSLAELQSMLGELQWVSKGTPVLRSSLHQLYLALRGHRDPRDTIELTSTQVQALKT |
| | | IQKALALNCRSRLVSQLPILALIILRPTGTTAVLFQTKQKWPLVWLHTPHPATSLRPWGQLLANAIITLDKYSL |
| | | QHYGQICKSFHHNISNQALTYYLHTSDQSSVAILLQHSHRFHNLGAQPSGPWRSLLQVPQIFQNIDVLRPPFII |
| | | SPVVIDHAPCLFSDGATSKAAFILWDKQVIHQQVLPLPSTCSAQAGELFGLLAGLQKSKPWPALNIFLDSKFLI |
| | | GHLRRMAWGAFLGPSTQCDLHARLFPLLQGKTVYVHHVRSHTLLQDPISRLNEATDALMLAPLLPL |
| HTLV2_ | 8,044 | HLPPPPQVDQFPLNLPERLQALNDLVSKALEAGHIEPYSGPGNNPVFPVKKPNGKWRFIHDLRATNAITTTLTS |
| P03363_ | | PSPGPPDLTSLPTALPHLQTIDLTDAFFQIPLPKQYQPYFAFTIPQPCNYGPGTRYAWTVLPQGFKNSPTLFQQ |
| 2mut | | QLAAVLNPMRKMFPTSTIVQYMDDILLASPTNEELQQLSQLTLQALTTHGLPISQEKTQQTPGQIRFLGQVISP |
| | | NHITYESTPTIPIKSQWTLTELQVILGEIQWVSKGTPILRKHLQSLYSALHPYRDPRACITLTPQQLHALHAIQ |
| | | QALQHNCRGRLNPALPLLGLISLSTSGTTSVIFQPKQNWPLAWLHTPHPPTSLCPWGHLLACTILTLDKYTLQH |
| | | YGQLCQSFHHNMSKQALCDFLRNSPHPSVGILIHHMGRFHNLGSQPSGPWKTLLHLPTLLQEPRLLRPIFTLSP |
| | | VVLDTAPCLFSDGSPQKAAYVLWDQTILQQDITPLPSHETHSAQKGELLALICGLRAAKPWPSLNIFLDSKYLI |
| | | KYLHSLAIGAFLGTSAHQTLQAALPPLLQGKTIYLHHVRSHTNLPDPISTFNEYTDSLILAPLVPL |
| JSRV_ | 8,045 | PLGTSDSPVTHADPIDWKSEEPVWVDQWPLTQEKLSAAQQLVQEQLRLGHIEPSTSAWNSPIFVIKKKSGKWRL |
| P31623 | | LQDLRKVNETMMHMGALQPGLPTPSAIPDKSYIIVIDLKDCFYTIPLAPQDCKRFAFSLPSVNFKEPMQRYQWR |
| | | VLPQGMTNSPTLCQKFVATAIAPVRQRFPQLYLVHYMDDILLAHTDEHLLYQAFSILKQHLSLNGLVIADEKIQ |
| | | THFPYNYLGFSLYPRVYNTQLVKLQTDHLKTLNDFQKLLGDINWIRPYLKLPTYTLQPLFDILKGDSDPASPRT |
| | | LSLEGRTALQSIEEAIRQQQITYCDYQRSWGLYILPTPRAPTGVLYQDKPLRWIYLSATPTKHLLPYYELVAKI |
| | | IAKGRHEAIQYFGMEPPFICVPYALEQQDWLFQFSDNWSIAFANYPGQITHHYPSDKLLQFASSHAFIFPKIVR |
| | | RQPIPEATLIFTDGSSNGTAALIINHQTYYAQTSFSSAQVVELFAVHQALLTVPTSFNLFTDSSYVVGALQMIE |
| | | TVPIIGTTSPEVLNLFTLIQQVLHCRQHPCFFGHIRAHSTLPGALVQGNHTADVLTKQVFFQS |
| JSRV_ | 8,046 | PLGTSDSPVTHADPIDWKSEEPVWVDQWPLTQEKLSAAQQLVQEQLRLGHIEPSTSAWNSPIFVIKKKSGKWRL |
| P31623_ | | LQDLRKVNETMMHMGALQPGLPTPSPIPDKSYIIVIDLKDCFYTIPLAPQDCKRFAFSLPSVNFKEPMQRYQWR |
| 2mutB | | VLPQGMTNSPTLCQKFVATAIAPVRQRFPQLYLVHYMDDILLAHTDEHLLYQAFSILKQHLSLNGLVIADEKIQ |
| | | THFPYNYLGFSLYPRVYNTQLVKLQTDHLKTLNDFQKLLGDINWIRPYLKLPTYTLQPLFDILKGDSDPASPRT |
| | | LSLEGRTALQSIEEAIRQQQITYCDYQRSWGLYILPTPRAPTGVLYQDKPLRWIYLSATPTKHLLPYYELVAKI |
| | | IAKGRHEAIQYFGMEPPFICVPYALEQQDWLFQFSDNWSIAFANYPGQITHHYPSDKLLQFASSHAFIFPKIVR |
| | | RQPIPEATLIFTDGSSNGTAALIINHQTYYAQTSFSSAQVVELFAVHQALLTVPTSFNLFTDSSYVVGALQMIE |
| | | TVPIIGTTSPEVLNLFTLIQQVLHCRQHPCFFGHIRAHSTLPGALVQGNHTADVLTKQVFFQS |
| KORV_ | 8,047 | TLGDQGSRGSDPLPEPRVTLTVEGIPTEFLVNTGAEHSVLTKPMGKMGSKRTVVAGATGSKVYPWTTKRLLKIG |
| Q9TTC1 | | QKQVTHSFLVIPECPAPLLGRDLLTKLKAQIQFSTEGPQVTWEDRPAMCLVLNLEEEYRLHEKPVPPSIDPSWL |
| | | QLFPMVWAEKAGMGLANQVPPVVVELKSDASPVAVRQYPMSKEAREGIRPHIQRFLDLGILVPCQSPWNTPLLP |
| | | VKKPGTNDYRPVQDLREVNKRVQDIHPTVPNPYNLLSSLPPSHTWYSVLDLKDAFFCLKLHPNSQPLFAFEWRD |
| | | PEKGNTGQLTWTRLPQGFKNSPTLFDEALHRDLASFRALNPQVVMLQYVDDLLVAAPTYRDCKEGTRRLLQELS |
| | | KLGYRVSAKKAQLCREEVTYLGYLLKGGKRWLTPARKATVMKIPTPTTPRQVREFLGTAGFCRLWIPGFASLAA |
| | | PLYPLTREKVPFTWTEAHQEAFGRIKEALLSAPALALPDLTKPFALYVDEKEGVARGVLTQTLGPWRRPVAYLS |
| | | KKLDPVASGWPTCLKAIAAVALLLKDADKLTLGQNVLVIAPHNLESIVRQPPDRWMTNARMTHYQSLLLNERVS |
| | | FAPPAILNPATLLPVESDDTPIHICSEILAEETGTRPDLRDQPLPGVPAWYTDGSSFIMDGRRQAGAAIVDNKR |
| | | TVWASNLPEGTSAQKAELIALTQALRLAEGKSINIYTDSRYAFATAHVHGAIYKQRGLLTSAGKDIKNKEEILA |
| | | LLEAIHLPKRVAIIHCPGHQRGTDPVATGNRKADEAAKQAAQSTRILTETTKN |
| KORV_ | 8,048 | TLGDQGSRGSDPLPEPRVTLTVEGIPTEFLVNTGAEHSVLTKPMGKMGSKRTVVAGATGSKVYPWTTKRLLKIG |
| Q9TTC1_ | | QKQVTHSFLVIPECPAPLLGRDLLTKLKAQIQFSTEGPQVTWEDRPAMCLVLNLEEEYRLHEKPVPPSIDPSWL |
| 3mut | | QLFPMVWAEKAGMGLANQVPPVVVELKSDASPVAVRQYPMSKEAREGIRPHIQRFLDLGILVPCQSPWNTPLLP |
| | | VKKPGTNDYRPVQDLREVNKRVQDIHPTVPNPYNLLSSLPPSHTWYSVLDLKDAFFCLKLHPNSQPLFAFEWRD |
| | | PEKGNTGQLTWTRLPQGFKNSPTLFNEALHRDLASFRALNPQVVMLQYVDDLLVAAPTYRDCKEGTRRLLQELS |
| | | KLGYRVSAKKAQLCREEVTYLGYLLKGGKRWLTPARKATVMKIPTPTTPRQVREFLGTAGFCRLWIPGFASLAA |
| | | PLYPLTRPKVPFTWTEAHQEAFGRIKEALLSAPALALPDLTKPFALYVDEKEGVARGVLTQTLGPWRRPVAYLS |
| | | KKLDPVASGWPTCLKAIAAVALLLKDADKLTLGQNVLVIAPHNLESIVRQPPDRWMTNARMTHYQSLLLNERVS |
| | | FAPPAILNPATLLPVESDDTPIHICSEILAEETGTRPDLRDQPLPGVPAWYTDGSSFIMDGRRQAGAAIVDNKR |
| | | TVWASNLPEGTSAQKAELIALTQALRLAEGKSINIYTDSRYAFATAHVHGAIYKQRGWLTSAGKDIKNKEEILA |
| | | LLEAIHLPKRVAIIHCPGHQRGTDPVATGNRKADEAAKQAAQSTRILTETTKN |
| KORV_ | 8,049 | TLGDQGSRGSDPLPEPRVTLTVEGIPTEFLVNTGAEHSVLTKPMGKMGSKRTVVAGATGSKVYPWTTKRLLKIG |
| Q9TTC1_ | | QKQVTHSFLVIPECPAPLLGRDLLTKLKAQIQFSTEGPQVTWEDRPAMCLVLNLEEEYRLHEKPVPPSIDPSWL |
| 3mutA | | QLFPMVWAEKAGMGLANQVPPVVVELKSDASPVAVRQYPMSKEAREGIRPHIQRFLDLGILVPCQSPWNTPLLP |
| | | VKKPGTNDYRPVQDLREVNKRVQDIHPTVPNPYNLLSSLPPSHTWYSVLDLKDAFFCLKLHPNSQPLFAFEWRD |
| | | PEKGNTGQLTWTRLPQGFKNSPTLFNEALHRDLASFRALNPQVVMLQYVDDLLVAAPTYRDCKEGTRRLLQELS |
| | | KLGYRVSAKKAQLCREEVTYLGYLLKGGKRWLTPARKATVMKIPTPTTPRQVREFLGKAGFCRLFIPGFASLAA |
| | | PLYPLTRPKVPFTWTEAHQEAFGRIKEALLSAPALALPDLTKPFALYVDEKEGVARGVLTQTLGPWRRPVAYLS |
| | | KKLDPVASGWPTCLKAIAAVALLLKDADKLTLGQNVLVIAPHNLESIVRQPPDRWMTNARMTHYQSLLLNERVS |
| | | FAPPAILNPATLLPVESDDTPIHICSEILAEETGTRPDLRDQPLPGVPAWYTDGSSFIMDGRRQAGAAIVDNKR |
| | | TVWASNLPEGTSAQKAELIALTQALRLAEGKSINIYTDSRYAFATAHVHGAIYKQRGWLTSAGKDIKNKEEILA |
| | | LLEAIHLPKRVAIIHCPGHQRGTDPVATGNRKADEAAKQAAQSTRILTETTKN |
| KORV_ | 8,050 | LLGRDLLTKLKAQIQFSTEGPQVTWEDRPAMCLVLNLEEEYRLHEKPVPPSIDPSWLQLFPMVWAEKAGMGLAN |
| Q9TTC1- | | QVPPVVVELKSDASPVAVRQYPMSKEAREGIRPHIQRFLDLGILVPCQSPWNTPLLPVKKPGTNDYRPVQDLRE |
| Pro | | VNKRVQDIHPTVPNPYNLLSSLPPSHTWYSVLDLKDAFFCLKLHPNSQPLFAFEWRDPEKGNTGQLTWTRLPQG |
| | | FKNSPTLFDEALHRDLASFRALNPQVVMLQYVDDLLVAAPTYRDCKEGTRRLLQELSKLGYRVSAKKAQLCREE |
| | | VTYLGYLLKGGKRWLTPARKATVMKIPTPTTPRQVREFLGTAGFCRLWIPGFASLAAPLYPLTREKVPFTWTEA |
| | | HQEAFGRIKEALLSAPALALPDLTKPFALYVDEKEGVARGVLTQTLGPWRRPVAYLSKKLDPVASGWPTCLKAI |
| | | AAVALLLKDADKLTLGQNVLVIAPHNLESIVRQPPDRWMTNARMTHYQSLLLNERVSFAPPAILNPATLLPVES |
| | | DDTPIHICSEILAEETGTRPDLRDQPLPGVPAWYTDGSSFIMDGRRQAGAAIVDNKRTVWASNLPEGTSAQKAE |
| | | LIALTQALRLAEGKSINIYTDSRYAFATAHVHGAIYKQRGLLTSAGKDIKNKEEILALLEAIHLPKRVAIIHCP |
| | | GHQRGTDPVATGNRKADEAAKQAAQSTRILTETTKN |
| KORV_ | 8,051 | LLGRDLLTKLKAQIQFSTEGPQVTWEDRPAMCLVLNLEEEYRLHEKPVPPSIDPSWLQLFPMVWAEKAGMGLAN |
| Q9TTC1- | | QVPPVVVELKSDASPVAVRQYPMSKEAREGIRPHIQRFLDLGILVPCQSPWNTPLLPVKKPGTNDYRPVQDLRE |
| Pro_ | | VNKRVQDIHPTVPNPYNLLSSLPPSHTWYSVLDLKDAFFCLKLHPNSQPLFAFEWRDPEKGNTGQLTWTRLPQG |
| 3mut | | FKNSPTLFNEALHRDLASFRALNPQVVMLQYVDDLLVAAPTYRDCKEGTRRLLQELSKLGYRVSAKKAQLCREE |
| | | VTYLGYLLKGGKRWLTPARKATVMKIPTPTTPRQVREFLGTAGFCRLWIPGFASLAAPLYPLTRPKVPFTWTEA |
| | | HQEAFGRIKEALLSAPALALPDLTKPFALYVDEKEGVARGVLTQTLGPWRRPVAYLSKKLDPVASGWPTCLKAI |
| | | AAVALLLKDADKLTLGQNVLVIAPHNLESIVRQPPDRWMTNARMTHYQSLLLNERVSFAPPAILNPATLLPVES |
| | | DDTPIHICSEILAEETGTRPDLRDQPLPGVPAWYTDGSSFIMDGRRQAGAAIVDNKRTVWASNLPEGTSAQKAE |
| | | LIALTQALRLAEGKSINIYTDSRYAFATAHVHGAIYKQRGWLTSAGKDIKNKEEILALLEAIHLPKRVAIIHCP |
| | | GHQRGTDPVATGNRKADEAAKQAAQSTRILTETTKN |
| KORV_ | 8,052 | LLGRDLLTKLKAQIQFSTEGPQVTWEDRPAMCLVLNLEEEYRLHEKPVPPSIDPSWLQLFPMVWAEKAGMGLAN |
| Q9TTC1- | | QVPPVVVELKSDASPVAVRQYPMSKEAREGIRPHIQRFLDLGILVPCQSPWNTPLLPVKKPGTNDYRPVQDLRE |
| Pro_ | | VNKRVQDIHPTVPNPYNLLSSLPPSHTWYSVLDLKDAFFCLKLHPNSQPLFAFEWRDPEKGNTGQLTWTRLPQG |
| 3mutA | | FKNSPTLFNEALHRDLASFRALNPQVVMLQYVDDLLVAAPTYRDCKEGTRRLLQELSKLGYRVSAKKAQLCREE |
| | | VTYLGYLLKGGKRWLTPARKATVMKIPTPTTPRQVREFLGKAGFCRLFIPGFASLAAPLYPLTRPKVPFTWTEA |
| | | HQEAFGRIKEALLSAPALALPDLTKPFALYVDEKEGVARGVLTQTLGPWRRPVAYLSKKLDPVASGWPTCLKAI |
| | | AAVALLLKDADKLTLGQNVLVIAPHNLESIVRQPPDRWMTNARMTHYQSLLLNERVSFAPPAILNPATLLPVES |
| | | DDTPIHICSEILAEETGTRPDLRDQPLPGVPAWYTDGSSFIMDGRRQAGAAIVDNKRTVWASNLPEGTSAQKAE |
| | | LIALTQALRLAEGKSINIYTDSRYAFATAHVHGAIYKQRGWLTSAGKDIKNKEEILALLEAIHLPKRVAIIHCP |
| | | GHQRGTDPVATGNRKADEAAKQAAQSTRILTETTKN |
| MLVAV_ | 8,053 | TLNLEDEYRLYETSAEPEVSPGSTWLSDFPQAWAETGGMGLAVRQAPLIIPLKATSTPVSIKQYPMSQEAKLGI |
| P03356 | | KPHIQRLLDQGILVPCQSPWNTPLLPVKKPGTNDYRPVQDLREVNKRVEDIHPTVPNPYNLLSGLPPSHRWYTV |
| | | LDLKDAFFCLRLHPTSQPLFAFEWRDPGMGISGQLTWTRLPQGFKNSPTLFDEALHRDLADFRIQHPDLILLQY |
| | | VDDILLAATSELDCQQGTRALLLTLGNLGYRASAKKAQLCQKQVKYLGYLLKEGQRWLTEARKETVMGQPTPKT |
| | | PRQLREFLGTAGFCRLWIPGFAEMAAPLYPLTKTGTLFNWGPDQQKAYQEIKQALLTAPALGLPDLTKPFELFV |
| | | DEKQGYAKGVLTQKLGPWRRPVAYLSKKLDPVAAGWPPCLRMVAAIAVLRKDAGKLTMGQPLVILAPHAVEALV |
| | | KQPPDRWLSNARMTHYQAMLLDTDRVQFGPVVALNPATLLPLPEEGAPHDCLEILAETHGTRPDLTDQPIPDAD |
| | | HTWYTDGSSFLQEGQRKAGAAVTTETEVIWARALPAGTSAQRAELIALTQALKMAEGKRLNVYTDSRYAFATAH |
| | | IHGEIYRRRGLLTSEGREIKNKSEILALLKALFLPKRLSIIHCLGHQKGDSAEARGNRLADQAAREAAIKTPPD |
| | | TSTLL |
| MLVAV_ | 8,054 | TLNLEDEYRLYETSAEPEVSPGSTWLSDFPQAWAETGGMGLAVRQAPLIIPLKATSTPVSIKQYPMSQEAKLGI |
| P03356_ | | KPHIQRLLDQGILVPCQSPWNTPLLPVKKPGTNDYRPVQDLREVNKRVEDIHPTVPNPYNLLSGLPPSHRWYTV |
| 3mut | | LDLKDAFFCLRLHPTSQPLFAFEWRDPGMGISGQLTWTRLPQGFKNSPTLFNEALHRDLADFRIQHPDLILLQY |
| | | VDDILLAATSELDCQQGTRALLLTLGNLGYRASAKKAQLCQKQVKYLGYLLKEGQRWLTEARKETVMGQPTPKT |
| | | PRQLREFLGTAGFCRLWIPGFAEMAAPLYPLTKPGTLFNWGPDQQKAYQEIKQALLTAPALGLPDLTKPFELFV |
| | | DEKQGYAKGVLTQKLGPWRRPVAYLSKKLDPVAAGWPPCLRMVAAIAVLRKDAGKLTMGQPLVILAPHAVEALV |
| | | KQPPDRWLSNARMTHYQAMLLDTDRVQFGPVVALNPATLLPLPEEGAPHDCLEILAETHGTRPDLTDQPIPDAD |
| | | HTWYTDGSSFLQEGQRKAGAAVTTETEVIWARALPAGTSAQRAELIALTQALKMAEGKRLNVYTDSRYAFATAH |
| | | IHGEIYRRRGWLTSEGREIKNKSEILALLKALFLPKRLSIIHCLGHQKGDSAEARGNRLADQAAREAAIKTPPD |
| | | TSTLL |
| MLVAV_ | 8,055 | TLNLEDEYRLYETSAEPEVSPGSTWLSDFPQAWAETGGMGLAVRQAPLIIPLKATSTPVSIKQYPMSQEAKLGI |
| P03356_ | | KPHIQRLLDQGILVPCQSPWNTPLLPVKKPGTNDYRPVQDLREVNKRVEDIHPTVPNPYNLLSGLPPSHRWYTV |
| 3mutA | | LDLKDAFFCLRLHPTSQPLFAFEWRDPGMGISGQLTWTRLPQGFKNSPTLFNEALHRDLADFRIQHPDLILLQY |
| | | VDDILLAATSELDCQQGTRALLLTLGNLGYRASAKKAQLCQKQVKYLGYLLKEGQRWLTEARKETVMGQPTPKT |
| | | PRQLREFLGKAGFCRLFIPGFAEMAAPLYPLTKPGTLFNWGPDQQKAYQEIKQALLTAPALGLPDLTKPFELFV |
| | | DEKQGYAKGVLTQKLGPWRRPVAYLSKKLDPVAAGWPPCLRMVAAIAVLRKDAGKLTMGQPLVILAPHAVEALV |
| | | KQPPDRWLSNARMTHYQAMLLDTDRVQFGPVVALNPATLLPLPEEGAPHDCLEILAETHGTRPDLTDQPIPDAD |
| | | HTWYTDGSSFLQEGQRKAGAAVTTETEVIWARALPAGTSAQRAELIALTQALKMAEGKRLNVYTDSRYAFATAH |
| | | IHGEIYRRRGWLTSEGREIKNKSEILALLKALFLPKRLSIIHCLGHQKGDSAEARGNRLADQAAREAAIKTPPD |
| | | TSTLL |
| MLVBM_ | 8,056 | TLGIEDEYRLHETSTEPDVSLGSTWLSDFPQAWAETGGMGLAVRQAPLIIPLKATSTPVSIQQYPMSHEARLGI |
| Q7SVK7 | | KPHIQRLLDQGILVPCQSPWNTPLLPVKKPGTNDYRPVQDLREVNKRVEDIHPTVPNPYNLLSGLPPSHQWYTV |
| | | LDLKDAFFCLRLHPTSQPLFAFEWRDPGMGISGQLTWTRLPQGFKNSPTLFDEALHRDLADFRIQHPDLILLQY |
| | | VDDILLAATSELDCQQGTRALLQTLGDLGYRASAKKAQICQKQVKYLGYLLREGQRWLTEARKETVMGQPVPKT |
| | | PRQLREFLGTAGFCRLWIPGFAEMAAPLYPLTKTGTLFSWGPDQQKAYQEIKQALLTAPALGLPDLTKPFELFV |
| | | DEKQGYAKGVLTQKLGPWRRPVAYLSKKLDPVAAGWPPCLRMVAAIAVLTKDAGKLTMGQPLVILAPHAVEALV |
| | | KQPPDRWLSNARMTHYQAMLLDTDRVQFGPVVALNPATLLPLPEEGAPHDCLEILAETHGTRPDLTDQPIPDAD |
| | | HTWYTDGSSFLQEGQRKAGAAVTTETEVIWAGALPAGTSAQRAELIALTQALKMAEGKRLNVYTDSRYAFATAH |
| | | IHGEIYRRRGLLTSEGREIKNKSEILALLKALFLPKRLSIIHCLGHQKGDSAEARGNRLADQAAREAAIKTPPD |
| | | TSTLL |
| MLVBM_ | 8,057 | TLGIEDEYRLHETSTEPDVSLGSTWLSDFPQAWAETGGMGLAVRQAPLIIPLKATSTPVSIQQYPMSHEARLGI |
| Q7SVK7 | | KPHIQRLLDQGILVPCQSPWNTPLLPVKKPGTNDYRPVQDLREVNKRVEDIHPTVPNPYNLLSGLPPSHQWYTV |
| | | LDLKDAFFCLRLHPTSQPLFAFEWRDPGMGISGQLTWTRLPQGFKNSPTLFDEALHRDLADFRIQHPDLILLQY |
| | | VDDILLAATSELDCQQGTRALLQTLGDLGYRASAKKAQICQKQVKYLGYLLREGQRWLTEARKETVMGQPVPKT |
| | | PRQLREFLGTAGFCRLWIPGFAEMAAPLYPLTKTGTLFSWGPDQQKAYQEIKQALLTAPALGLPDLTKPFELFV |
| | | DEKQGYAKGVLTQKLGPWRRPVAYLSKKLDPVAAGWPPCLRMVAAIAVLTKDAGKLTMGQPLVILAPHAVEALV |
| | | KQPPDRWLSNARMTHYQAMLLDTDRVQFGPVVALNPATLLPLPEEGAPHDCLEILAETHGTRPDLTDQPIPDAD |
| | | HTWYTDGSSFLQEGQRKAGAAVTTETEVIWAGALPAGTSAQRAELIALTQALKMAEGKRLNVYTDSRYAFATAH |
| | | IHGEIYRRRGLLTSEGREIKNKSEILALLKALFLPKRLSIIHCLGHQKGDSAEARGNRLADQAAREAAIKTPPD |
| | | TSTLL |
| MLVBM_ | 8,058 | TLGIEDEYRLHETSTEPDVSLGSTWLSDFPQAWAETGGMGLAVRQAPLIIPLKATSTPVSIQQYPMSHEARLGI |
| Q7SVK7_ | | KPHIQRLLDQGILVPCQSPWNTPLLPVKKPGTNDYRPVQDLREVNKRVEDIHPTVPNPYNLLSGLPPSHQWYTV |
| 3mut | | LDLKDAFFCLRLHPTSQPLFAFEWRDPGMGISGQLTWTRLPQGFKNSPTLFNEALHRDLADFRIQHPDLILLQY |
| | | VDDILLAATSELDCQQGTRALLQTLGDLGYRASAKKAQICQKQVKYLGYLLREGQRWLTEARKETVMGQPVPKT |
| | | PRQLREFLGTAGFCRLWIPGFAEMAAPLYPLTKPGTLFSWGPDQQKAYQEIKQALLTAPALGLPDLTKPFELFV |
| | | DEKQGYAKGVLTQKLGPWRRPVAYLSKKLDPVAAGWPPCLRMVAAIAVLTKDAGKLTMGQPLVILAPHAVEALV |
| | | KQPPDRWLSNARMTHYQAMLLDTDRVQFGPVVALNPATLLPLPEEGAPHDCLEILAETHGTRPDLTDQPIPDAD |
| | | HTWYTDGSSFLQEGQRKAGAAVTTETEVIWAGALPAGTSAQRAELIALTQALKMAEGKRLNVYTDSRYAFATAH |
| | | IHGEIYRRRGWLTSEGREIKNKSEILALLKALFLPKRLSIIHCLGHQKGDSAEARGNRLADQAAREAAIKTPPD |
| | | TSTLL |
| MLVBM_ | 8,059 | TLGIEDEYRLHETSTEPDVSLGSTWLSDFPQAWAETGGMGLAVRQAPLIIPLKATSTPVSIQQYPMSHEARLGI |
| Q7SVK7_ | | KPHIQRLLDQGILVPCQSPWNTPLLPVKKPGTNDYRPVQDLREVNKRVEDIHPTVPNPYNLLSGLPPSHQWYTV |
| 3mut | | LDLKDAFFCLRLHPTSQPLFAFEWRDPGMGISGQLTWTRLPQGFKNSPTLFNEALHRDLADFRIQHPDLILLQY |
| | | VDDILLAATSELDCQQGTRALLQTLGDLGYRASAKKAQICQKQVKYLGYLLREGQRWLTEARKETVMGQPVPKT |
| | | PRQLREFLGTAGFCRLWIPGFAEMAAPLYPLTKPGTLFSWGPDQQKAYQEIKQALLTAPALGLPDLTKPFELFV |
| | | DEKQGYAKGVLTQKLGPWRRPVAYLSKKLDPVAAGWPPCLRMVAAIAVLTKDAGKLTMGQPLVILAPHAVEALV |
| | | KQPPDRWLSNARMTHYQAMLLDTDRVQFGPVVALNPATLLPLPEEGAPHDCLEILAETHGTRPDLTDQPIPDAD |
| | | HTWYTDGSSFLQEGQRKAGAAVTTETEVIWAGALPAGTSAQRAELIALTQALKMAEGKRLNVYTDSRYAFATAH |
| | | IHGEIYRRRGWLTSEGREIKNKSEILALLKALFLPKRLSIIHCLGHQKGDSAEARGNRLADQAAREAAIKTPPD |
| | | TSTLL |
| MLVBM_ | 8,060 | LGIEDEYRLHETSTEPDVSLGSTWLSDFPQAWAETGGMGLAVRQAPLIIPLKATSTPVSIQQYPMSHEARLGIK |
| Q7SVK7_ | | PHIQRLLDQGILVPCQSPWNTPLLPVKKPGTNDYRPVQDLREVNKRVEDIHPTVPNPYNLLSGLPPSHQWYTVL |
| 3mutA_ | | DLKDAFFCLRLHPTSQPLFAFEWRDPGMGISGQLTWTRLPQGFKNSPTLFNEALHRDLADFRIQHPDLILLQYV |
| WS | | DDILLAATSELDCQQGTRALLQTLGDLGYRASAKKAQICQKQVKYLGYLLREGQRWLTEARKETVMGQPVPKTP |
| | | RQLREFLGKAGFCRLFIPGFAEMAAPLYPLTKPGTLFSWGPDQQKAYQEIKQALLTAPALGLPDLTKPFELFVD |
| | | EKQGYAKGVLTQKLGPWRRPVAYLSKKLDPVAAGWPPCLRMVAAIAVLTKDAGKLTMGQPLVILAPHAVEALVK |
| | | QPPDRWLSNARMTHYQAMLLDTDRVQFGPVVALNPATLLPLPEEGAPHDCLEILAETHGTRPDLTDQPIPDADH |
| | | TWYTDGSSFLQEGQRKAGAAVTTETEVIWAGALPAGTSAQRAELIALTQALKMAEGKRLNVYTDSRYAFATAHI |
| | | HGEIYRRRGWLTSEGREIKNKSEILALLKALFLPKRLSIIHCLGHQKGDSAEARGNRLADQAAREAAIKTPPDT |
| | | STLLI |
| MLVBM_ | 8,061 | LGIEDEYRLHETSTEPDVSLGSTWLSDFPQAWAETGGMGLAVRQAPLIIPLKATSTPVSIQQYPMSHEARLGIK |
| Q7SVK7_ | | PHIQRLLDQGILVPCQSPWNTPLLPVKKPGTNDYRPVQDLREVNKRVEDIHPTVPNPYNLLSGLPPSHQWYTVL |
| 3mutAWS | | DLKDAFFCLRLHPTSQPLFAFEWRDPGMGISGQLTWTRLPQGFKNSPTLFNEALHRDLADFRIQHPDLILLQYV |
| | | DDILLAATSELDCQQGTRALLQTLGDLGYRASAKKAQICQKQVKYLGYLLREGQRWLTEARKETVMGQPVPKTP |
| | | RQLREFLGKAGFCRLFIPGFAEMAAPLYPLTKPGTLFSWGPDQQKAYQEIKQALLTAPALGLPDLTKPFELFVD |
| | | EKQGYAKGVLTQKLGPWRRPVAYLSKKLDPVAAGWPPCLRMVAAIAVLTKDAGKLTMGQPLVILAPHAVEALVK |
| | | QPPDRWLSNARMTHYQAMLLDTDRVQFGPWVALNPATLLPLPEEGAPHDCLEILAETHGTRPDLTDQPIPDADH |
| | | TWYTDGSSFLQEGQRKAGAAVTTETEVIWAGALPAGTSAQRAELIALTQALKMAEGKRLNVYTDSRYAFATAHI |
| | | HGEIYRRRGWLTSEGREIKNKSEILALLKALFLPKRLSIIHCLGHQKGDSAEARGNRLADQAAREAAIKTPPDT |
| | | STLLI |
| MLVCB_ | 8,062 | TLNIEDEYRLHETSKEPDVSLGSTWLSDFPQAWAETGGMGLAVRQAPLIIPLKATSTPVSIKQYPMSQEARLG |
| P08361 | | IKPHIQRLLDQGILVPCQSPWNTPLLPVKKPGTNDYRPVQDLREVNKRVEDIHPTVPNPYNLLSGLPPSHQWY |
| | | TVLDLKDAFFCLRLHPTSQPLFAFEWRDPEMGISGQLTWTRLPQGFKNSPTLFDEALHRDLAGFRIQHPDLIL |
| | | LQYVDDLLLAATSELDCQQGTRALLQTLGDLGYRASAKKAQICQKQVKYLGYLLKEGQRWLTEARKETVMGQP |
| | | IPKTPRQLREFLGTAGFCRLWIPGFAEMAAPLYPLTKTGTLFNWGPDQQKAFQEIKQALLTAPALGLPDLTKP |
| | | FELFVDEKQGYAKGVLTQKLGPWRRPVAYLSKKLDPVAAGWPPCLRMVAAIAVLTKDAGKLTMGQPLVILAPH |
| | | AVEALVKQPPDRWLSNARMTHYQALLLDTDRVQFGPVVALNPATLLPLPEEGLQHDCLDILAEAHGTRSDLMD |
| | | QPLPDADHTWYTDGSSFLQEGQRKAGAAVTTETEVIWARALPAGTSAQRAELIALTQALKMAEGKKLNVYTDS |
| | | RYAFATAHIHGEIYRRRGLLTSEGKEIKNKDEILALLKALFLPKRLSIIHCPGHQKGNSAEARGNRMADQAAR |
| | | EVATRETPETSTLL |
| MLVCB_ | 8,063 | TLNIEDEYRLHETSKEPDVSLGSTWLSDFPQAWAETGGMGLAVRQAPLIIPLKATSTPVSIKQYPMSQEARLGI |
| P08361_ | | KPHIQRLLDQGILVPCQSPWNTPLLPVKKPGTNDYRPVQDLREVNKRVEDIHPTVPNPYNLLSGLPPSHQWYTV |
| 3mut | | LDLKDAFFCLRLHPTSQPLFAFEWRDPEMGISGQLTWTRLPQGFKNSPTLFNEALHRDLAGFRIQHPDLILLQY |
| | | VDDLLLAATSELDCQQGTRALLQTLGDLGYRASAKKAQICQKQVKYLGYLLKEGQRWLTEARKETVMGQPIPKT |
| | | PRQLREFLGTAGFCRLWIPGFAEMAAPLYPLTKPGTLFNWGPDQQKAFQEIKQALLTAPALGLPDLTKPFELFV |
| | | DEKQGYAKGVLTQKLGPWRRPVAYLSKKLDPVAAGWPPCLRMVAAIAVLTKDAGKLTMGQPLVILAPHAVEALV |
| | | KQPPDRWLSNARMTHYQALLLDTDRVQFGPVVALNPATLLPLPEEGLQHDCLDILAEAHGTRSDLMDQPLPDAD |
| | | HTWYTDGSSFLQEGQRKAGAAVTTETEVIWARALPAGTSAQRAELIALTQALKMAEGKKLNVYTDSRYAFATAH |
| | | IHGEIYRRRGWLTSEGKEIKNKDEILALLKALFLPKRLSIIHCPGHQKGNSAEARGNRMADQAAREVATRETPE |
| | | TSTLL |
| MLVCB_ | 8,064 | TLNIEDEYRLHETSKEPDVSLGSTWLSDFPQAWAETGGMGLAVRQAPLIIPLKATSTPVSIKQYPMSQEARLGI |
| P08361_ | | KPHIQRLLDQGILVPCQSPWNTPLLPVKKPGTNDYRPVQDLREVNKRVEDIHPTVPNPYNLLSGLPPSHQWYTV |
| 3mutA | | LDLKDAFFCLRLHPTSQPLFAFEWRDPEMGISGQLTWTRLPQGFKNSPTLFNEALHRDLAGFRIQHPDLILLQY |
| | | VDDLLLAATSELDCQQGTRALLQTLGDLGYRASAKKAQICQKQVKYLGYLLKEGQRWLTEARKETVMGQPIPKT |
| | | PRQLREFLGKAGFCRLFIPGFAEMAAPLYPLTKPGTLFNWGPDQQKAFQEIKQALLTAPALGLPDLTKPFELFV |
| | | DEKQGYAKGVLTQKLGPWRRPVAYLSKKLDPVAAGWPPCLRMVAAIAVLTKDAGKLTMGQPLVILAPHAVEALV |
| | | KQPPDRWLSNARMTHYQALLLDTDRVQFGPVVALNPATLLPLPEEGLQHDCLDILAEAHGTRSDLMDQPLPDAD |
| | | HTWYTDGSSFLQEGQRKAGAAVTTETEVIWARALPAGTSAQRAELIALTQALKMAEGKKLNVYTDSRYAFATAH |
| | | IHGEIYRRRGWLTSEGKEIKNKDEILALLKALFLPKRLSIIHCPGHQKGNSAEARGNRMADQAAREVATRETPE |
| | | TSTLL |
| MLVF5_ | 8,065 | TLNIEDEYRLHETSKGPDVPLGSTWLSDFPQAWAETGGMGLAFRQAPLIISLKATSTPVSIKQYPMSQEARLGI |
| P26810 | | KPHIQRLLDQGILVPCQSPWNTPLLPVKKPGTNDYRPVQDLREVNKRVEDIHPTVPNPYNLLSGLPPSHQWYTV |
| | | LDLKDAFFCLRLHPTSQSLFAFEWKDPEMGISGQLTWTRLPQGFKNSPTLFDEALHRDLADFRIQHPDLILLQY |
| | | VDDLLLAATSELDCQQGTRALLQTLGDLGYRASAKKAQICQKQVKYLGYLLKEGQRWLTEARKETVMGQPTPKT |
| | | PRQLREFLGTAGLCRLWIPGFAEMAAPLYPLTKTGTLFKWGPDQQKAYQEIKQALLTAPALGLPDLTKPFELFV |
| | | DEKQGYAKGVLTQKLGPWRRPVAYLSKKLDPVAAGWPPCLRMVAAIAVLTKDVGKLTMGQPLVILAPHAVEALV |
| | | KQPPDRWLSNARMTHYQALLLDTDRVQFGPIVALNPATLLPLPEEGLQHDCLDILAEAHGTRPDLTDQPLPDAD |
| | | HTWYTDGSSFLQEGQRRAGAAVTTETEVIWAKALPAGTSAQRAELIALTQALKMAAGKKLNVYTDSRYAFATAH |
| | | IHGEIYRRRGLLTSEGKEIKNKDEILALLKALFLPKRLSIIHCPGHQKGNHAEARGNRMADQAAREVATRETPE |
| | | TSTLL |
| MLVF5_ | 8,066 | TLNIEDEYRLHETSKGPDVPLGSTWLSDFPQAWAETGGMGLAFRQAPLIISLKATSTPVSIKQYPMSQEARLGI |
| P26810_ | | KPHIQRLLDQGILVPCQSPWNTPLLPVKKPGTNDYRPVQDLREVNKRVEDIHPTVPNPYNLLSGLPPSHQWYTV |
| 3mut | | LDLKDAFFCLRLHPTSQSLFAFEWKDPEMGISGQLTWTRLPQGFKNSPTLFNEALHRDLADFRIQHPDLILLQY |
| | | VDDLLLAATSELDCQQGTRALLQTLGDLGYRASAKKAQICQKQVKYLGYLLKEGQRWLTEARKETVMGQPTPKT |
| | | PRQLREFLGTAGLCRLWIPGFAEMAAPLYPLTKPGTLFKWGPDQQKAYQEIKQALLTAPALGLPDLTKPFELFV |
| | | DEKQGYAKGVLTQKLGPWRRPVAYLSKKLDPVAAGWPPCLRMVAAIAVLTKDVGKLTMGQPLVILAPHAVEALV |
| | | KQPPDRWLSNARMTHYQALLLDTDRVQFGPIVALNPATLLPLPEEGLQHDCLDILAEAHGTRPDLTDQPLPDAD |
| | | HTWYTDGSSFLQEGQRRAGAAVTTETEVIWAKALPAGTSAQRAELIALTQALKMAAGKKLNVYTDSRYAFATAH |
| | | IHGEIYRRRGWLTSEGKEIKNKDEILALLKALFLPKRLSIIHCPGHQKGNHAEARGNRMADQAAREVATRETPE |
| | | TSTLL |
| MLVF5_ | 8,067 | TLNIEDEYRLHETSKGPDVPLGSTWLSDFPQAWAETGGMGLAFRQAPLIISLKATSTPVSIKQYPMSQEARLGI |
| P26810_ | | KPHIQRLLDQGILVPCQSPWNTPLLPVKKPGTNDYRPVQDLREVNKRVEDIHPTVPNPYNLLSGLPPSHQWYTV |
| 3mutA | | LDLKDAFFCLRLHPTSQSLFAFEWKDPEMGISGQLTWTRLPQGFKNSPTLFNEALHRDLADFRIQHPDLILLQY |
| | | VDDLLLAATSELDCQQGTRALLQTLGDLGYRASAKKAQICQKQVKYLGYLLKEGQRWLTEARKETVMGQPTPKT |
| | | PRQLREFLGKAGLCRLFIPGFAEMAAPLYPLTKPGTLFKWGPDQQKAYQEIKQALLTAPALGLPDLTKPFELFV |
| | | DEKQGYAKGVLTQKLGPWRRPVAYLSKKLDPVAAGWPPCLRMVAAIAVLTKDVGKLTMGQPLVILAPHAVEALV |
| | | KQPPDRWLSNARMTHYQALLLDTDRVQFGPIVALNPATLLPLPEEGLQHDCLDILAEAHGTRPDLTDQPLPDAD |
| | | HTWYTDGSSFLQEGQRRAGAAVTTETEVIWAKALPAGTSAQRAELIALTQALKMAAGKKLNVYTDSRYAFATAH |
| | | IHGEIYRRRGWLTSEGKEIKNKDEILALLKALFLPKRLSIIHCPGHQKGNHAEARGNRMADQAAREVATRETPE |
| | | TSTLL |
| MLVFF_ | 8,068 | TLNIEDEYRLHETSKGPDVPLGSTWLSDFPQAWAETGGMGLAVRQAPLIIPLKATSTPVSIKQYPMSQEARLGI |
| P26809_ | | KPHIQRLLDQGILVPCQSPWNTPLLPVKKPGTNDYRPVQDLREVNKRVEDIHPTVPNPYNLLSGLPPSHQWYTV |
| 3mut | | LDLKDAFFCLRLHPTSQSLFAFEWRDPEMGISGQLTWTRLPQGFKNSPTLFNEALHRDLADFRIQHPDLILLQY |
| | | VDDLLLAATSELDCQQGTRALLQTLGDLGYRASAKKAQICQKQVKYLGYLLKEGQRWLTEARKETVMGQPTPKT |
| | | PRQLREFLGTAGFCRLWIPGFAEMAAPLYPLTKPGTLFEWGPDQQKAYQEIKQALLTAPALGLPDLTKPFELFV |
| | | DEKQGYAKGVLTQKLGPWRRPVAYLSKKLDPVAAGWPPCLRMVAAIAVLTKDAGKLTMGQPLVILAPHAVEALV |
| | | KQPPDRWLSNARMTHYQALLLDTDRVQFGPIVALNPATLLPLPEEGLQHDCLDILAEAHGTRPDLTDQPLPDAD |
| | | HTWYTDGSSFLQEGQRKAGAAVTTETEVVWAKALPAGTSAQRAELIALTQALKMAEGKKLNVYTDSRYAFATAH |
| | | IHGEIYRRRGWLTSEGKEIKNKDEILALLKALFLPKRLSIIHCPGHQKGNRAEARGNRMADQAAREVATRETPE |
| | | TSTLL |
| MLVFF_ | 8,069 | TLNIEDEYRLHETSKGPDVPLGSTWLSDFPQAWAETGGMGLAVRQAPLIIPLKATSTPVSIKQYPMSQEARLGI |
| P26809_ | | KPHIQRLLDQGILVPCQSPWNTPLLPVKKPGTNDYRPVQDLREVNKRVEDIHPTVPNPYNLLSGLPPSHQWYTV |
| 3mutA | | LDLKDAFFCLRLHPTSQSLFAFEWRDPEMGISGQLTWTRLPQGFKNSPTLFNEALHRDLADFRIQHPDLILLQY |
| | | VDDLLLAATSELDCQQGTRALLQTLGDLGYRASAKKAQICQKQVKYLGYLLKEGQRWLTEARKETVMGQPTPKT |
| | | PRQLREFLGKAGFCRLFIPGFAEMAAPLYPLTKPGTLFEWGPDQQKAYQEIKQALLTAPALGLPDLTKPFELFV |
| | | DEKQGYAKGVLTQKLGPWRRPVAYLSKKLDPVAAGWPPCLRMVAAIAVLTKDAGKLTMGQPLVILAPHAVEALV |
| | | KQPPDRWLSNARMTHYQALLLDTDRVQFGPIVALNPATLLPLPEEGLQHDCLDILAEAHGTRPDLTDQPLPDAD |
| | | HTWYTDGSSFLQEGQRKAGAAVTTETEVVWAKALPAGTSAQRAELIALTQALKMAEGKKLNVYTDSRYAFATAH |
| | | IHGEIYRRRGWLTSEGKEIKNKDEILALLKALFLPKRLSIIHCPGHQKGNRAEARGNRMADQAAREVATRETPE |
| | | TSTLL |
| MLVMS_ | 8,070 | TLNIEDEHRLHETSKEPDVSLGSTWLSDFPQAWAETGGMGLAVRQAPLIIPLKATSTPVSIKQYPMSQEARLGI |
| P03355 | | KPHIQRLLDQGILVPCQSPWNTPLLPVKKPGTNDYRPVQDLREVNKRVEDIHPTVPNPYNLLSGLPPSHQWYTV |
| | | LDLKDAFFCLRLHPTSQPLFAFEWRDPEMGISGQLTWTRLPQGFKNSPTLFDEALHRDLADFRIQHPDLILLQY |
| | | VDDLLLAATSELDCQQGTRALLQTLGNLGYRASAKKAQICQKQVKYLGYLLKEGQRWLTEARKETVMGQPTPKT |
| | | PRQLREFLGTAGFCRLWIPGFAEMAAPLYPLTKTGTLFNWGPDQQKAYQEIKQALLTAPALGLPDLTKPFELFV |
| | | DEKQGYAKGVLTQKLGPWRRPVAYLSKKLDPVAAGWPPCLRMVAAIAVLTKDAGKLTMGQPLVILAPHAVEALV |
| | | KQPPDRWLSNARMTHYQALLLDTDRVQFGPVVALNPATLLPLPEEGLQHNCLDILAEAHGTRPDLTDQPLPDAD |
| | | HTWYTDGSSLLQEGQRKAGAAVTTETEVIWAKALPAGTSAQRAELIALTQALKMAEGKKLNVYTDSRYAFATAH |
| | | IHGEIYRRRGLLTSEGKEIKNKDEILALLKALFLPKRLSIIHCPGHQKGHSAEARGNRMADQAARKAAITETPD |
| | | TSTLL |
| MLVMS_ | 8,071 | TLNIEDEHRLHETSKEPDVSLGSTWLSDFPQAWAETGGMGLAVRQAPLIIPLKATSTPVSIKQYPMSQEARLGI |
| P03355 | | KPHIQRLLDQGILVPCQSPWNTPLLPVKKPGTNDYRPVQDLREVNKRVEDIHPTVPNPYNLLSGLPPSHQWYTV |
| | | LDLKDAFFCLRLHPTSQPLFAFEWRDPEMGISGQLTWTRLPQGFKNSPTLFDEALHRDLADFRIQHPDLILLQY |
| | | VDDLLLAATSELDCQQGTRALLQTLGNLGYRASAKKAQICQKQVKYLGYLLKEGQRWLTEARKETVMGQPTPKT |
| | | PRQLREFLGTAGFCRLWIPGFAEMAAPLYPLTKTGTLFNWGPDQQKAYQEIKQALLTAPALGLPDLTKPFELFV |
| | | DEKQGYAKGVLTQKLGPWRRPVAYLSKKLDPVAAGWPPCLRMVAAIAVLTKDAGKLTMGQPLVILAPHAVEALV |
| | | KQPPDRWLSNARMTHYQALLLDTDRVQFGPVVALNPATLLPLPEEGLQHNCLDILAEAHGTRPDLTDQPLPDAD |
| | | HTWYTDGSSLLQEGQRKAGAAVTTETEVIWAKALPAGTSAQRAELIALTQALKMAEGKKLNVYTDSRYAFATAH |
| | | IHGEIYRRRGLLTSEGKEIKNKDEILALLKALFLPKRLSIIHCPGHQKGHSAEARGNRMADQAARKAAITETPD |
| | | TSTLL |
| MLVMS_ | 8,072 | TLNIEDEHRLHETSKEPDVSLGSTWLSDFPQAWAETGGMGLAVRQAPLIIPLKATSTPVSIKQYPMSQEARLGI |
| P03355_ | | KPHIQRLLDQGILVPCQSPWNTPLLPVKKPGTNDYRPVQDLREVNKRVEDIHPTVPNPYNLLSGLPPSHQWYTV |
| 3mut | | LDLKDAFFCLRLHPTSQPLFAFEWRDPEMGISGQLTWTRLPQGFKNSPTLFNEALHRDLADFRIQHPDLILLQY |
| | | VDDLLLAATSELDCQQGTRALLQTLGNLGYRASAKKAQICQKQVKYLGYLLKEGQRWLTEARKETVMGQPTPKT |
| | | PRQLREFLGTAGFCRLWIPGFAEMAAPLYPLTKPGTLFNWGPDQQKAYQEIKQALLTAPALGLPDLTKPFELFV |
| | | DEKQGYAKGVLTQKLGPWRRPVAYLSKKLDPVAAGWPPCLRMVAAIAVLTKDAGKLTMGQPLVILAPHAVEALV |
| | | KQPPDRWLSNARMTHYQALLLDTDRVQFGPVVALNPATLLPLPEEGLQHNCLDILAEAHGTRPDLTDQPLPDAD |
| | | HTWYTDGSSLLQEGQRKAGAAVTTETEVIWAKALPAGTSAQRAELIALTQALKMAEGKKLNVYTDSRYAFATAH |
| | | IHGEIYRRRGWLTSEGKEIKNKDEILALLKALFLPKRLSIIHCPGHQKGHSAEARGNRMADQAARKAAITETPD |
| | | TSTLL |
| MLVMS_ | 8,073 | TLNIEDEHRLHETSKEPDVSLGSTWLSDFPQAWAETGGMGLAVRQAPLIIPLKATSTPVSIKQYPMSQEARLGI |
| P03355_ | | KPHIQRLLDQGILVPCQSPWNTPLLPVKKPGTNDYRPVQDLREVNKRVEDIHPTVPNPYNLLSGLPPSHQWYTV |
| 3mut | | LDLKDAFFCLRLHPTSQPLFAFEWRDPEMGISGQLTWTRLPQGFKNSPTLFNEALHRDLADFRIQHPDLILLQY |
| | | VDDLLLAATSELDCQQGTRALLQTLGNLGYRASAKKAQICQKQVKYLGYLLKEGQRWLTEARKETVMGQPTPKT |
| | | PRQLREFLGTAGFCRLWIPGFAEMAAPLYPLTKPGTLFNWGPDQQKAYQEIKQALLTAPALGLPDLTKPFELFV |
| | | DEKQGYAKGVLTQKLGPWRRPVAYLSKKLDPVAAGWPPCLRMVAAIAVLTKDAGKLTMGQPLVILAPHAVEALV |
| | | KQPPDRWLSNARMTHYQALLLDTDRVQFGPVVALNPATLLPLPEEGLQHNCLDILAEAHGTRPDLTDQPLPDAD |
| | | HTWYTDGSSLLQEGQRKAGAAVTTETEVIWAKALPAGTSAQRAELIALTQALKMAEGKKLNVYTDSRYAFATAH |
| | | IHGEIYRRRGWLTSEGKEIKNKDEILALLKALFLPKRLSIIHCPGHQKGHSAEARGNRMADQAARKAAITETPD |
| | | TSTLL |
| MLVMS_ | 8,074 | TLNIEDEHRLHETSKEPDVSLGSTWLSDFPQAWAETGGMGLAVRQAPLIIPLKATSTPVSIKQYPMSQEARLGI |
| P03355_ | | KPHIQRLLDQGILVPCQSPWNTPLLPVKKPGTNDYRPVQDLREVNKRVEDIHPTVPNPYNLLSGLPPSHQWYTV |
| 3mutAWS | | LDLKDAFFCLRLHPTSQPLFAFEWRDPEMGISGQLTWTRLPQGFKNSPTLFNEALHRDLADFRIQHPDLILLQY |
| | | VDDLLLAATSELDCQQGTRALLQTLGNLGYRASAKKAQICQKQVKYLGYLLKEGQRWLTEARKETVMGQPTPKT |
| | | PRQLREFLGKAGFCRLFIPGFAEMAAPLYPLTKPGTLFNWGPDQQKAYQEIKQALLTAPALGLPDLTKPFELFV |
| | | DEKQGYAKGVLTQKLGPWRRPVAYLSKKLDPVAAGWPPCLRMVAAIAVLTKDAGKLTMGQPLVILAPHAVEALV |
| | | KQPPDRWLSNARMTHYQALLLDTDRVQFGPVVALNPATLLPLPEEGLQHNCLDILAEAHGTRPDLTDQPLPDAD |
| | | HTWYTDGSSLLQEGQRKAGAAVTTETEVIWAKALPAGTSAQRAELIALTQALKMAEGKKLNVYTDSRYAFATAH |
| | | IHGEIYRRRGWLTSEGKEIKNKDEILALLKALFLPKRLSIIHCPGHQKGHSAEARGNRMADQAARKAAITETPD |
| | | TSTLL |
| MLVMS_ | 8,075 | TLNIEDEHRLHETSKEPDVSLGSTWLSDFPQAWAETGGMGLAVRQAPLIIPLKATSTPVSIKQYPMSQEARLGI |
| P03355_ | | KPHIQRLLDQGILVPCQSPWNTPLLPVKKPGTNDYRPVQDLREVNKRVEDIHPTVPNPYNLLSGLPPSHQWYTV |
| 3mutAWS | | LDLKDAFFCLRLHPTSQPLFAFEWRDPEMGISGQLTWTRLPQGFKNSPTLFNEALHRDLADFRIQHPDLILLQY |
| | | VDDLLLAATSELDCQQGTRALLQTLGNLGYRASAKKAQICQKQVKYLGYLLKEGQRWLTEARKETVMGQPTPKT |
| | | PRQLREFLGKAGFCRLFIPGFAEMAAPLYPLTKPGTLFNWGPDQQKAYQEIKQALLTAPALGLPDLTKPFELFV |
| | | DEKQGYAKGVLTQKLGPWRRPVAYLSKKLDPVAAGWPPCLRMVAAIAVLTKDAGKLTMGQPLVILAPHAVEALV |
| | | KQPPDRWLSNARMTHYQALLLDTDRVQFGPVVALNPATLLPLPEEGLQHNCLDILAEAHGTRPDLTDQPLPDAD |
| | | HTWYTDGSSLLQEGQRKAGAAVTTETEVIWAKALPAGTSAQRAELIALTQALKMAEGKKLNVYTDSRYAFATAH |
| | | IHGEIYRRRGWLTSEGKEIKNKDEILALLKALFLPKRLSIIHCPGHQKGHSAEARGNRMADQAARKAAITETPD |
| | | TSTLL |
| MLVMS_ | 8,076 | TLNIEDEYRLHETSKEPDVSLGSTWLSDFPQAWAETGGMGLAVRQAPLIIPLKATSTPVSIKQYPMSQEARLGI |
| P03355_ | | KPHIQRLLDQGILVPCQSPWNTPLLPVKKPGTNDYRPVQDLREVNKRVEDIHPTVPNPYNLLSGLPPSHQWYTV |
| PLV919 | | LDLKDAFFCLRLHPTSQPLFAFEWRDPEMGISGQLTWTRLPQGFKNSPTLFNEALHRDLADFRIQHPDLILLQY |
| | | VDDLLLAATSELDCQQGTRALLQTLGNLGYRASAKKAQICQKQVKYLGYLLKEGQRWLTEARKETVMGQPTPKT |
| | | PRQLREFLGKAGFCRLFIPGFAEMAAPLYPLTKPGTLFNWGPDQQKAYQEIKQALLTAPALGLPDLTKPFELFV |
| | | DEKQGYAKGVLTQKLGPWRRPVAYLSKKLDPVAAGWPPCLRMVAAIAVLTKDAGKLTMGQPLVILAPHAVEALV |
| | | KQPPDRWLSNARMTHYQALLLDTDRVQFGPVVALNPATLLPLPEEGLQHNCLDILAEAHGTRPDLTDQPLPDAD |
| | | HTWYTDGSSLLQEGQRKAGAAVTTETEVIWAKALPAGTSAQRAELIALTQALKMAEGKKLNVYTDSRYAFATAH |
| | | IHGEIYRRRGWLTSEGKEIKNKDEILALLKALFLPKRLSIIHCPGHQKGHSAEARGNRMADQAARKAAITETPD |
| | | TSTLLIENSSPSGGSKRTADGSEFE |
| MLVMS_ | 8,077 | TLNIEDEYRLHETSKEPDVSLGSTWLSDFPQAWAETGGMGLAVRQAPLIIPLKATSTPVSIKQYPMSQEARLGI |
| P03355_ | | KPHIQRLLDQGILVPCQSPWNTPLLPVKKPGTNDYRPVQDLREVNKRVEDIHPTVPNPYNLLSGLPPSHQWYTV |
| PLV919 | | LDLKDAFFCLRLHPTSQPLFAFEWRDPEMGISGQLTWTRLPQGFKNSPTLFNEALHRDLADFRIQHPDLILLQY |
| | | VDDLLLAATSELDCQQGTRALLQTLGNLGYRASAKKAQICQKQVKYLGYLLKEGQRWLTEARKETVMGQPTPKT |
| | | PRQLREFLGKAGFCRLFIPGFAEMAAPLYPLTKPGTLFNWGPDQQKAYQEIKQALLTAPALGLPDLTKPFELFV |
| | | DEKQGYAKGVLTQKLGPWRRPVAYLSKKLDPVAAGWPPCLRMVAAIAVLTKDAGKLTMGQPLVILAPHAVEALV |
| | | KQPPDRWLSNARMTHYQALLLDTDRVQFGPVVALNPATLLPLPEEGLQHNCLDILAEAHGTRPDLTDQPLPDAD |
| | | HTWYTDGSSLLQEGQRKAGAAVTTETEVIWAKALPAGTSAQRAELIALTQALKMAEGKKLNVYTDSRYAFATAH |
| | | IHGEIYRRRGWLTSEGKEIKNKDEILALLKALFLPKRLSIIHCPGHQKGHSAEARGNRMADQAARKAAITETPD |
| | | TSTLLIENSSPSGGSKRTADGSEFE |
| MLVRD_ | 8,078 | TLNIEDEYRLHEISTEPDVSPGSTWLSDFPQAWAETGGMGLAVRQAPLIIPLKATSTPVSIKQYPMSQEAKLGI |
| P11227 | | KPHIQRLLDQGILVPCQSPWNTPLLPVKKPGTNDYRPVQGLREVNKRVEDIHPTVPNPYNLLSGLPTSHRWYTV |
| | | LDLKDAFFCLRLHPTSQPLFASEWRDPGMGISGQLTWTRLPQGFKNSPTLFDEALHRGLADFRIQHPDLILLQY |
| | | VDDLLLAATSELDCQQGTRALLKTLGNLGYRASAKKAQICQKQVKYLGYLLREGQRWLTEARKETVMGQPTPKT |
| | | PRQLREFLGTAGFCRLWIPRFAEMAAPLYPLTKTGTLFNWGPDQQKAYHEIKQALLTAPALGLPDLTKPFELFV |
| | | DEKQGYAKGVLTQKLGPWRRPVAYLSKKLDPVAAGWPPCLRMVAAIAVLTKDAGKLTMGQPLVILAPHAVEALV |
| | | KQPPDRWLSNARMTHYQAMLLDTDRVQFGPVVALNPATLLPLPEEGAPHDCLEILAETHGTEPDLTDQPIPDAD |
| | | HTWYTDGSSFLQEGQRKAGAAVTTETEVIWARALPAGTSAQRAELIALTQALKMAEGKRLNVYTDSRYAFATAH |
| | | IHGEIYKRRGLLTSEGREIKNKSEILALLKALFLPKRLSIIHCLGHQKGDSAEARGNRLADQAAREAAIKTPPD |
| | | TSTLL |
| MLVRD_ | 8,079 | TLNIEDEYRLHEISTEPDVSPGSTWLSDFPQAWAETGGMGLAVRQAPLIIPLKATSTPVSIKQYPMSQEAKLGI |
| P11227_ | | KPHIQRLLDQGILVPCQSPWNTPLLPVKKPGTNDYRPVQGLREVNKRVEDIHPTVPNPYNLLSGLPTSHRWYTV |
| 3mut | | LDLKDAFFCLRLHPTSQPLFASEWRDPGMGISGQLTWTRLPQGFKNSPTLFNEALHRGLADFRIQHPDLILLQY |
| | | VDDLLLAATSELDCQQGTRALLKTLGNLGYRASAKKAQICQKQVKYLGYLLREGQRWLTEARKETVMGQPTPKT |
| | | PRQLREFLGTAGFCRLWIPRFAEMAAPLYPLTKPGTLFNWGPDQQKAYHEIKQALLTAPALGLPDLTKPFELFV |
| | | DEKQGYAKGVLTQKLGPWRRPVAYLSKKLDPVAAGWPPCLRMVAAIAVLTKDAGKLTMGQPLVILAPHAVEALV |
| | | KQPPDRWLSNARMTHYQAMLLDTDRVQFGPVVALNPATLLPLPEEGAPHDCLEILAETHGTEPDLTDQPIPDAD |
| | | HTWYTDGSSFLQEGQRKAGAAVTTETEVIWARALPAGTSAQRAELIALTQALKMAEGKRLNVYTDSRYAFATAH |
| | | IHGEIYKRRGWLTSEGREIKNKSEILALLKALFLPKRLSIIHCLGHQKGDSAEARGNRLADQAAREAAIKTPPD |
| | | TSTLL |
| MMTVB_ | 8,080 | WVQEISDSRPMLHIYLNGRRFLGLLNTGADKTCIAGRDWPANWPIHQTESSLQGLGMACGVARSSQPLRWQHED |
| P03365 | | KSGIIHPFVIPTLPFTLWGRDIMKDIKVRLMTDSPDDSQDLMIGAIESNLFADQISWKSDQPVWLNQWPLKQEK |
| | | VLQALQQLVTEQLQLGHLEESNSPWNTPVFVIKKKSGKWRLLQDLRAVNATMHDMGALQPGLPSPVAVPKGWEII |
| | | PIIDLQDCFFNIKLHPEDCKRFAFSVPSPNFKRPYQRFQWKVLPQGMKNSPTLCQKFVDKAILTVRDKYQDSYIV |
| | | DHYMDDILLAHPSRSIVDEILTSMIQALNKHGLVVSTEKIQKYDNLKYLGTHIQGDSVSYQKLQIRTDKLRTLND |
| | | KFQKLLGNINWIRPFLKLTTGELKPLFEILNGDSNPISTRKLTPEACKALQLMNERLSTARVKRLDLSQPWSLCI |
| | | HLKTEYTPTACLWQDGVVEWIHLPHISPKVITPYDIFCTQLIIKGRHRSKELFSKDPDYIVVPYTKVQFDLLLQE |
| | | IKEDWPISLLGFLGEVHFHLPKDPLLTFTLQTAIIFPHMTSTTPLEKGIVIFTDGSANGRSVTYIQGREPIIKEN |
| | | TTQNTAQQAEIVAVITAFEEVSQPFNLYTDSKYVTGLFPEIETATLSPRTKIYTELKHLQRLIHKRQEKFYIGHI |
| | | RGHTGLPGPLAQGNAYADSLTRILT |
| MMTVB_ | 8,081 | WVQEISDSRPMLHIYLNGRRFLGLLNTGADKTCIAGRDWPANWPIHQTESSLQGLGMACGVARSSQPLRWQHED |
| P03365 | | KSGIIHPFVIPTLPFTLWGRDIMKDIKVRLMTDSPDDSQDLMIGAIESNLFADQISWKSDQPVWLNQWPLKQEK |
| | | LQALQQLVTEQLQLGHLEESNSPWNTPVFVIKKKSGKWRLLQDLRAVNATMHDMGALQPGLPSPVAVPKGWEII |
| | | IIDLQDCFFNIKLHPEDCKRFAFSVPSPNFKRPYQRFQWKVLPQGMKNSPTLCQKFVDKAILTVRDKYQDSYIV |
| | | HYMDDILLAHPSRSIVDEILTSMIQALNKHGLVVSTEKIQKYDNLKYLGTHIQGDSVSYQKLQIRTDKLRTLND |
| | | FQKLLGNINWIRPFLKLTTGELKPLFEILNGDSNPISTRKLTPEACKALQLMNERLSTARVKRLDLSQPWSLCI |
| | | LKTEYTPTACLWQDGVVEWIHLPHISPKVITPYDIFCTQLIIKGRHRSKELFSKDPDYIVVPYTKVQFDLLLQE |
| | | KEDWPISLLGFLGEVHFHLPKDPLLTFTLQTAIIFPHMTSTTPLEKGIVIFTDGSANGRSVTYIQGREPIIKEN |
| | | TQNTAQQAEIVAVITAFEEVSQPFNLYTDSKYVTGLFPEIETATLSPRTKIYTELKHLQRLIHKRQEKFYIGHI |
| | | RGHTGLPGPLAQGNAYADSLTRILT |
| MMTVB_ | 8,082 | WVQEISDSRPMLHIYLNGRRFLGLLNTGADKTCIAGRDWPANWPIHQTESSLQGLGMACGVARSSQPLRWQHED |
| P03365_ | | KSGIIHPFVIPTLPFTLWGRDIMKDIKVRLMTDSPDDSQDLMIGAIESNLFADQISWKSDQPVWLNQWPLKQEK |
| 2mut | | LQALQQLVTEQLQLGHLEESNSPWNTPVFVIKKKSGKWRLLQDLRAVNATMHDMGALQPGLPSPVAVPKGWEII |
| | | IIDLQDCFFNIKLHPEDCKRFAFSVPSPNFKRPYQRFQWKVLPQGMKNSPTLCQKFVDKAILTVRDKYQDSYIV |
| | | HYMDDILLAHPSRSIVDEILTSMIQALNKHGLVVSTEKIQKYDNLKYLGTHIQGDSVSYQKLQIRTDKLRTLND |
| | | FQKLLGNINWIRPFLKLTTGELKPLFEILNPDSNPISTRKLTPEACKALQLMNERLSTARVKRLDLSQPWSLCI |
| | | LKTEYTPTACLWQDGVVEWIHLPHISPKVITPYDIFCTQLIIKGRHRSKELFSKDPDYIVVPYTKVQFDLLLQE |
| | | KEDWPISLLGFLGEVHFHLPKDPLLTFTLQTAIIFPHMTSTTPLEKGIVIFTDGSANGRSVTYIQGREPIIKEN |
| | | TQNTAQQAEIVAVITAFEEVSQPFNLYTDSKYVTGLFPEIETATLSPRTKIYTELKHLQRLIHKRQEKFYIGHI |
| | | RGHTGLPGPLAQGNAYADSLTRILT |
| MMTVB_ | 8,083 | VQEISDSRPMLHIYLNGRRFLGLLDTGADKTCIAGRDWPANWPIHQTESSLQGLGMACGVARSSQPLRWQHEDK |
| P03365_ | | SGIIHPFVIPTLPFTLWGRDIMKDIKVRLMTDSPDDSQDLMIGAIESNLFADQISWKSDQPVWLNQWPLKQEKL |
| 2mut_ | | QALQQLVTEQLQLGHLEESNSPWNTPVFVIKKKSGKWRLLQDLRAVNATMHDMGALQPGLPSPVAVPKGWEIII |
| WS | | IDLQDCFFNIKLHPEDCKRFAFSVPSPNFKRPYQRFQWKVLPQGMKNSPTLCQKFVDKAILTVRDKYQDSYIVH |
| | | YMDDILLAHPSRSIVDEILTSMIQALNKHGLVVSTEKIQKYDNLKYLGTHIQGDSVSYQKLQIRTDKLRTLNDF |
| | | QKLLGNINWIRPFLKLTTGELKPLFEILNPDSNPISTRKLTPEACKALQLMNERLSTARVKRLDLSQPWSLCIL |
| | | KTEYTPTACLWQDGVVEWIHLPHISPKVITPYDIFCTQLIIKGRHRSKELFSKDPDYIVVPYTKVQFDLLLQEK |
| | | EDWPISLLGFLGEVHFHLPKDPLLTFTLQTAIIFPHMTSTTPLEKGIVIFTDGSANGRSVTYIQGREPIIKENT |
| | | QNTAQQAEIVAVITAFEEVSQPFNLYTDSKYVTGLFPEIETATLSPRTKIYTELKHLQRLIHKRQEKFYIGHIR |
| | | GHTGLPGPLAQGNAYADSLTRILTA |
| MMTVB_ | 8,084 | VQEISDSRPMLHIYLNGRRFLGLLDTGADKTCIAGRDWPANWPIHQTESSLQGLGMACGVARSSQPLRWQHEDK |
| P03365_ | | SGIIHPFVIPTLPFTLWGRDIMKDIKVRLMTDSPDDSQDLMIGAIESNLFADQISWKSDQPVWLNQWPLKQEKL |
| 2mut_ | | QALQQLVTEQLQLGHLEESNSPWNTPVFVIKKKSGKWRLLQDLRAVNATMHDMGALQPGLPSPVAVPKGWEIII |
| WS | | IDLQDCFFNIKLHPEDCKRFAFSVPSPNFKRPYQRFQWKVLPQGMKNSPTLCQKFVDKAILTVRDKYQDSYIVH |
| | | YMDDILLAHPSRSIVDEILTSMIQALNKHGLVVSTEKIQKYDNLKYLGTHIQGDSVSYQKLQIRTDKLRTLNDF |
| | | QKLLGNINWIRPFLKLTTGELKPLFEILNPDSNPISTRKLTPEACKALQLMNERLSTARVKRLDLSQPWSLCIL |
| | | KTEYTPTACLWQDGVVEWIHLPHISPKVITPYDIFCTQLIIKGRHRSKELFSKDPDYIVVPYTKVQFDLLLQEK |
| | | EDWPISLLGFLGEVHFHLPKDPLLTFTLQTAIIFPHMTSTTPLEKGIVIFTDGSANGRSVTYIQGREPIIKENT |
| | | QNTAQQAEIVAVITAFEEVSQPFNLYTDSKYVTGLFPEIETATLSPRTKIYTELKHLQRLIHKRQEKFYIGHIR |
| | | GHTGLPGPLAQGNAYADSLTRILTA |
| MMTVB_ | 8,085 | WVQEISDSRPMLHIYLNGRRFLGLLNTGADKTCIAGRDWPANWPIHQTESSLQGLGMACGVARSSQPLRWQHED |
| P03365_ | | KSGIIHPFVIPTLPFTLWGRDIMKDIKVRLMTDSPDDSQDLMIGAIESNLFADQISWKSDQPVWLNQWPLKQEK |
| 2mutB | | LQALQQLVTEQLQLGHLEESNSPWNTPVFVIKKKSGKWRLLQDLRAVNATMHDMGALQPGLPSPVAPPKGWEII |
| | | IIDLQDCFFNIKLHPEDCKRFAFSVPSPNFKRPYQRFQWKVLPQGMKNSPTLCQKFVDKAILTVRDKYQDSYIV |
| | | HYMDDILLAHPSRSIVDEILTSMIQALNKHGLVVSTEKIQKYDNLKYLGTHIQGDSVSYQKLQIRTDKLRTLND |
| | | FQKLLGNINWIRPFLKLTTGELKPLFEILNPDSNPISTRKLTPEACKALQLMNERLSTARVKRLDLSQPWSLCI |
| | | LKTEYTPTACLWQDGVVEWIHLPHISPKVITPYDIFCTQLIIKGRHRSKELFSKDPDYIVVPYTKVQFDLLLQE |
| | | KEDWPISLLGFLGEVHFHLPKDPLLTFTLQTAIIFPHMTSTTPLEKGIVIFTDGSANGRSVTYIQGREPIIKEN |
| | | TQNTAQQAEIVAVITAFEEVSQPFNLYTDSKYVTGLFPEIETATLSPRTKIYTELKHLQRLIHKRQEKFYIGHI |
| | | RGHTGLPGPLAQGNAYADSLTRILT |
| MMTVB_ | 8,086 | WVQEISDSRPMLHIYLNGRRFLGLLNTGADKTCIAGRDWPANWPIHQTESSLQGLGMACGVARSSQPLRWQHED |
| P03365_ | | KSGIIHPFVIPTLPFTLWGRDIMKDIKVRLMTDSPDDSQDLMIGAIESNLFADQISWKSDQPVWLNQWPLKQEK |
| 2mutB | | LQALQQLVTEQLQLGHLEESNSPWNTPVFVIKKKSGKWRLLQDLRAVNATMHDMGALQPGLPSPVAPPKGWEII |
| | | IIDLQDCFFNIKLHPEDCKRFAFSVPSPNFKRPYQRFQWKVLPQGMKNSPTLCQKFVDKAILTVRDKYQDSYIV |
| | | HYMDDILLAHPSRSIVDEILTSMIQALNKHGLVVSTEKIQKYDNLKYLGTHIQGDSVSYQKLQIRTDKLRTLND |
| | | FQKLLGNINWIRPFLKLTTGELKPLFEILNPDSNPISTRKLTPEACKALQLMNERLSTARVKRLDLSQPWSLCI |
| | | LKTEYTPTACLWQDGVVEWIHLPHISPKVITPYDIFCTQLIIKGRHRSKELFSKDPDYIVVPYTKVQFDLLLQE |
| | | KEDWPISLLGFLGEVHFHLPKDPLLTFTLQTAIIFPHMTSTTPLEKGIVIFTDGSANGRSVTYIQGREPIIKEN |
| | | TQNTAQQAEIVAVITAFEEVSQPFNLYTDSKYVTGLFPEIETATLSPRTKIYTELKHLQRLIHKRQEKFYIGHI |
| | | RGHTGLPGPLAQGNAYADSLTRILT |
| MMTVB_ | 8,087 | VQEISDSRPMLHIYLNGRRFLGLLDTGADKTCIAGRDWPANWPIHQTESSLQGLGMACGVARSSQPLRWQHEDK |
| P03365_ | | SGIIHPFVIPTLPFTLWGRDIMKDIKVRLMTDSPDDSQDLMIGAIESNLFADQISWKSDQPVWLNQWPLKQEKL |
| 2mutB_ | | QALQQLVTEQLQLGHLEESNSPWNTPVFVIKKKSGKWRLLQDLRAVNATMHDMGALQPGLPSPPAVPKGWEIII |
| WS | | IDLQDCFFNIKLHPEDCKRFAFSVPSPNFKRPYQRFQWKVLPQGMKNSPTLCQKFVDKAILTVRDKYQDSYIVH |
| | | YMDDILLAHPSRSIVDEILTSMIQALNKHGLVVSTEKIQKYDNLKYLGTHIQGDSVSYQKLQIRTDKLRTLNDF |
| | | QKLLGNINWIRPFLKLTTGELKPLFEILNPDSNPISTRKLTPEACKALQLMNERLSTARVKRLDLSQPWSLCIL |
| | | KTEYTPTACLWQDGVVEWIHLPHISPKVITPYDIFCTQLIIKGRHRSKELFSKDPDYIVVPYTKVQFDLLLQEK |
| | | EDWPISLLGFLGEVHFHLPKDPLLTFTLQTAIIFPHMTSTTPLEKGIVIFTDGSANGRSVTYIQGREPIIKENT |
| | | QNTAQQAEIVAVITAFEEVSQPFNLYTDSKYVTGLFPEIETATLSPRTKIYTELKHLQRLIHKRQEKFYIGHIR |
| | | GHTGLPGPLAQGNAYADSLTRILTA |
| MMTVB_ | 8,088 | VQEISDSRPMLHIYLNGRRFLGLLDTGADKTCIAGRDWPANWPIHQTESSLQGLGMACGVARSSQPLRWQHEDK |
| P03365_ | | SGIIHPFVIPTLPFTLWGRDIMKDIKVRLMTDSPDDSQDLMIGAIESNLFADQISWKSDQPVWLNQWPLKQEKL |
| 2mutBWS | | QALQQLVTEQLQLGHLEESNSPWNTPVFVIKKKSGKWRLLQDLRAVNATMHDMGALQPGLPSPPAVPKGWEIII |
| | | IDLQDCFFNIKLHPEDCKRFAFSVPSPNFKRPYQRFQWKVLPQGMKNSPTLCQKFVDKAILTVRDKYQDSYIVH |
| | | YMDDILLAHPSRSIVDEILTSMIQALNKHGLVVSTEKIQKYDNLKYLGTHIQGDSVSYQKLQIRTDKLRTLNDF |
| | | QKLLGNINWIRPFLKLTTGELKPLFEILNPDSNPISTRKLTPEACKALQLMNERLSTARVKRLDLSQPWSLCIL |
| | | KTEYTPTACLWQDGVVEWIHLPHISPKVITPYDIFCTQLIIKGRHRSKELFSKDPDYIVVPYTKVQFDLLLQEK |
| | | EDWPISLLGFLGEVHFHLPKDPLLTFTLQTAIIFPHMTSTTPLEKGIVIFTDGSANGRSVTYIQGREPIIKENT |
| | | QNTAQQAEIVAVITAFEEVSQPFNLYTDSKYVTGLFPEIETATLSPRTKIYTELKHLQRLIHKRQEKFYIGHIR |
| | | GHTGLPGPLAQGNAYADSLTRILTA |
| MMTVB_ | 8,089 | VQEISDSRPMLHIYLNGRRFLGLLDTGADKTCIAGRDWPANWPIHQTESSLQGLGMACGVARSSQPLRWQHEDK |
| P03365_ | | SGIIHPFVIPTLPFTLWGRDIMKDIKVRLMTDSPDDSQDLMIGAIESNLFADQISWKSDQPVWLNQWPLKQEKL |
| WS | | QALQQLVTEQLQLGHLEESNSPWNTPVFVIKKKSGKWRLLQDLRAVNATMHDMGALQPGLPSPVAVPKGWEIII |
| | | IDLQDCFFNIKLHPEDCKRFAFSVPSPNFKRPYQRFQWKVLPQGMKNSPTLCQKFVDKAILTVRDKYQDSYIVH |
| | | YMDDILLAHPSRSIVDEILTSMIQALNKHGLVVSTEKIQKYDNLKYLGTHIQGDSVSYQKLQIRTDKLRTLNDF |
| | | QKLLGNINWIRPFLKLTTGELKPLFEILNGDSNPISTRKLTPEACKALQLMNERLSTARVKRLDLSQPWSLCIL |
| | | KTEYTPTACLWQDGVVEWIHLPHISPKVITPYDIFCTQLIIKGRHRSKELFSKDPDYIVVPYTKVQFDLLLQEK |
| | | EDWPISLLGFLGEVHFHLPKDPLLTFTLQTAIIFPHMTSTTPLEKGIVIFTDGSANGRSVTYIQGREPIIKENT |
| | | QNTAQQAEIVAVITAFEEVSQPFNLYTDSKYVTGLFPEIETATLSPRTKIYTELKHLQRLIHKRQEKFYIGHIR |
| | | GHTGLPGPLAQGNAYADSLTRILTA |
| MMTVB_ | 8,090 | VQEISDSRPMLHIYLNGRRFLGLLDTGADKTCIAGRDWPANWPIHQTESSLQGLGMACGVARSSQPLRWQHEDK |
| P03365_ | | SGIIHPFVIPTLPFTLWGRDIMKDIKVRLMTDSPDDSQDLMIGAIESNLFADQISWKSDQPVWLNQWPLKQEKL |
| WS | | QALQQLVTEQLQLGHLEESNSPWNTPVFVIKKKSGKWRLLQDLRAVNATMHDMGALQPGLPSPVAVPKGWEIII |
| | | IDLQDCFFNIKLHPEDCKRFAFSVPSPNFKRPYQRFQWKVLPQGMKNSPTLCQKFVDKAILTVRDKYQDSYIVH |
| | | YMDDILLAHPSRSIVDEILTSMIQALNKHGLVVSTEKIQKYDNLKYLGTHIQGDSVSYQKLQIRTDKLRTLNDF |
| | | QKLLGNINWIRPFLKLTTGELKPLFEILNGDSNPISTRKLTPEACKALQLMNERLSTARVKRLDLSQPWSLCIL |
| | | KTEYTPTACLWQDGVVEWIHLPHISPKVITPYDIFCTQLIIKGRHRSKELFSKDPDYIVVPYTKVQFDLLLQEK |
| | | EDWPISLLGFLGEVHFHLPKDPLLTFTLQTAIIFPHMTSTTPLEKGIVIFTDGSANGRSVTYIQGREPIIKENT |
| | | QNTAQQAEIVAVITAFEEVSQPFNLYTDSKYVTGLFPEIETATLSPRTKIYTELKHLQRLIHKRQEKFYIGHIR |
| | | GHTGLPGPLAQGNAYADSLTRILTA |
| MMTVB_ | 8,091 | GRDIMKDIKVRLMTDSPDDSQDLMIGAIESNLFADQISWKSDQPVWLNQWPLKQEKLQALQQLVTEQLQLGHLE |
| P03365- | | ESNSPWNTPVFVIKKKSGKWRLLQDLRAVNATMHDMGALQPGLPSPVAVPKGWEIIIIDLQDCFFNIKLHPEDC |
| Pro | | KRFAFSVPSPNFKRPYQRFQWKVLPQGMKNSPTLCQKFVDKAILTVRDKYQDSYIVHYMDDILLAHPSRSIVDE |
| | | ILTSMIQALNKHGLVVSTEKIQKYDNLKYLGTHIQGDSVSYQKLQIRTDKLRTLNDFQKLLGNINWIRPFLKLT |
| | | TGELKPLFEILNGDSNPISTRKLTPEACKALQLMNERLSTARVKRLDLSQPWSLCILKTEYTPTACLWQDGVVE |
| | | WIHLPHISPKVITPYDIFCTQLIIKGRHRSKELFSKDPDYIVVPYTKVQFDLLLQEKEDWPISLLGFLGEVHFH |
| | | LPKDPLLTFTLQTAIIFPHMTSTTPLEKGIVIFTDGSANGRSVTYIQGREPIIKENTQNTAQQAEIVAVITAFE |
| | | EVSQPFNLYTDSKYVTGLFPEIETATLSPRTKIYTELKHLQRLIHKRQEKFYIGHIRGHTGLPGPLAQGNAYAD |
| | | SLTRILT |
| MMTVB_ | 8,092 | GRDIMKDIKVRLMTDSPDDSQDLMIGAIESNLFADQISWKSDQPVWLNQWPLKQEKLQALQQLVTEQLQLGHLE |
| P03365- | | ESNSPWNTPVFVIKKKSGKWRLLQDLRAVNATMHDMGALQPGLPSPVAVPKGWEIIIIDLQDCFFNIKLHPEDC |
| Pro | | KRFAFSVPSPNFKRPYQRFQWKVLPQGMKNSPTLCQKFVDKAILTVRDKYQDSYIVHYMDDILLAHPSRSIVDE |
| | | ILTSMIQALNKHGLVVSTEKIQKYDNLKYLGTHIQGDSVSYQKLQIRTDKLRTLNDFQKLLGNINWIRPFLKLT |
| | | TGELKPLFEILNGDSNPISTRKLTPEACKALQLMNERLSTARVKRLDLSQPWSLCILKTEYTPTACLWQDGVVE |
| | | WIHLPHISPKVITPYDIFCTQLIIKGRHRSKELFSKDPDYIVVPYTKVQFDLLLQEKEDWPISLLGFLGEVHFH |
| | | LPKDPLLTFTLQTAIIFPHMTSTTPLEKGIVIFTDGSANGRSVTYIQGREPIIKENTQNTAQQAEIVAVITAFE |
| | | EVSQPFNLYTDSKYVTGLFPEIETATLSPRTKIYTELKHLQRLIHKRQEKFYIGHIRGHTGLPGPLAQGNAYAD |
| | | SLTRILT |
| MMTVB_ | 8,093 | GRDIMKDIKVRLMTDSPDDSQDLMIGAIESNLFADQISWKSDQPVWLNQWPLKQEKLQALQQLVTEQLQLGHLE |
| P03365- | | ESNSPWNTPVFVIKKKSGKWRLLQDLRAVNATMHDMGALQPGLPSPVAVPKGWEIIIIDLQDCFFNIKLHPEDC |
| Pro_ | | KRFAFSVPSPNFKRPYQRFQWKVLPQGMKNSPTLCQKFVDKAILTVRDKYQDSYIVHYMDDILLAHPSRSIVDE |
| 2mut | | ILTSMIQALNKHGLVVSTEKIQKYDNLKYLGTHIQGDSVSYQKLQIRTDKLRTLNDFQKLLGNINWIRPFLKLT |
| | | TGELKPLFEILNPDSNPISTRKLTPEACKALQLMNERLSTARVKRLDLSQPWSLCILKTEYTPTACLWQDGVVE |
| | | WIHLPHISPKVITPYDIFCTQLIIKGRHRSKELFSKDPDYIVVPYTKVQFDLLLQEKEDWPISLLGFLGEVHFH |
| | | LPKDPLLTFTLQTAIIFPHMTSTTPLEKGIVIFTDGSANGRSVTYIQGREPIIKENTQNTAQQAEIVAVITAFE |
| | | EVSQPFNLYTDSKYVTGLFPEIETATLSPRTKIYTELKHLQRLIHKRQEKFYIGHIRGHTGLPGPLAQGNAYAD |
| | | SLTRILT |
| MMTVB_ | 8,094 | GRDIMKDIKVRLMTDSPDDSQDLMIGAIESNLFADQISWKSDQPVWLNQWPLKQEKLQALQQLVTEQLQLGHLE |
| P03365- | | ESNSPWNTPVFVIKKKSGKWRLLQDLRAVNATMHDMGALQPGLPSPVAVPKGWEIIIIDLQDCFFNIKLHPEDC |
| Pro_ | | KRFAFSVPSPNFKRPYQRFQWKVLPQGMKNSPTLCQKFVDKAILTVRDKYQDSYIVHYMDDILLAHPSRSIVDE |
| 2mut | | ILTSMIQALNKHGLVVSTEKIQKYDNLKYLGTHIQGDSVSYQKLQIRTDKLRTLNDFQKLLGNINWIRPFLKLT |
| | | TGELKPLFEILNPDSNPISTRKLTPEACKALQLMNERLSTARVKRLDLSQPWSLCILKTEYTPTACLWQDGVVE |
| | | WIHLPHISPKVITPYDIFCTQLIIKGRHRSKELFSKDPDYIVVPYTKVQFDLLLQEKEDWPISLLGFLGEVHFH |
| | | LPKDPLLTFTLQTAIIFPHMTSTTPLEKGIVIFTDGSANGRSVTYIQGREPIIKENTQNTAQQAEIVAVITAFE |
| | | EVSQPFNLYTDSKYVTGLFPEIETATLSPRTKIYTELKHLQRLIHKRQEKFYIGHIRGHTGLPGPLAQGNAYAD |
| | | SLTRILT |
| MMTVB_ | 8,095 | GRDIMKDIKVRLMTDSPDDSQDLMIGAIESNLFADQISWKSDQPVWLNQWPLKQEKLQALQQLVTEQLQLGHLE |
| P03365- | | ESNSPWNTPVFVIKKKSGKWRLLQDLRAVNATMHDMGALQPGLPSPVAPPKGWEIIIIDLQDCFFNIKLHPEDC |
| Pro_ | | KRFAFSVPSPNFKRPYQRFQWKVLPQGMKNSPTLCQKFVDKAILTVRDKYQDSYIVHYMDDILLAHPSRSIVDE |
| 2mutB | | ILTSMIQALNKHGLVVSTEKIQKYDNLKYLGTHIQGDSVSYQKLQIRTDKLRTLNDFQKLLGNINWIRPFLKLT |
| | | TGELKPLFEILNPDSNPISTRKLTPEACKALQLMNERLSTARVKRLDLSQPWSLCILKTEYTPTACLWQDGVVE |
| | | WIHLPHISPKVITPYDIFCTQLIIKGRHRSKELFSKDPDYIVVPYTKVQFDLLLQEKEDWPISLLGFLGEVHFH |
| | | LPKDPLLTFTLQTAIIFPHMTSTTPLEKGIVIFTDGSANGRSVTYIQGREPIIKENTQNTAQQAEIVAVITAFE |
| | | EVSQPFNLYTDSKYVTGLFPEIETATLSPRTKIYTELKHLQRLIHKRQEKFYIGHIRGHTGLPGPLAQGNAYAD |
| | | SLTRILT |
| MMTVB_ | 8,096 | GRDIMKDIKVRLMTDSPDDSQDLMIGAIESNLFADQISWKSDQPVWLNQWPLKQEKLQALQQLVTEQLQLGHLE |
| P03365- | | ESNSPWNTPVFVIKKKSGKWRLLQDLRAVNATMHDMGALQPGLPSPVAPPKGWEIIIIDLQDCFFNIKLHPEDC |
| Pro_ | | KRFAFSVPSPNFKRPYQRFQWKVLPQGMKNSPTLCQKFVDKAILTVRDKYQDSYIVHYMDDILLAHPSRSIVDE |
| 2mutB | | ILTSMIQALNKHGLVVSTEKIQKYDNLKYLGTHIQGDSVSYQKLQIRTDKLRTLNDFQKLLGNINWIRPFLKLT |
| | | TGELKPLFEILNPDSNPISTRKLTPEACKALQLMNERLSTARVKRLDLSQPWSLCILKTEYTPTACLWQDGVVE |
| | | WIHLPHISPKVITPYDIFCTQLIIKGRHRSKELFSKDPDYIVVPYTKVQFDLLLQEKEDWPISLLGFLGEVHFH |
| | | LPKDPLLTFTLQTAIIFPHMTSTTPLEKGIVIFTDGSANGRSVTYIQGREPIIKENTQNTAQQAEIVAVITAFE |
| | | EVSQPFNLYTDSKYVTGLFPEIETATLSPRTKIYTELKHLQRLIHKRQEKFYIGHIRGHTGLPGPLAQGNAYAD |
| | | SLTRILT |
| MPMV_ | 8,097 | LTAAIDILAPQQCAEPITWKSDEPVWVDQWPLTNDKLAAAQQLVQEQLEAGHITESSSPWNTPIFVIKKKSGKW |
| P07572 | | RLLQDLRAVNATMVLMGALQPGLPSPVAIPQGYLKIIIDLKDCFFSIPLHPSDQKRFAFSLPSTNFKEPMQRFQ |
| | | WKVLPQGMANSPTLCQKYVATAIHKVRHAWKQMYIIHYMDDILIAGKDGQQVLQCFDQLKQELTAAGLHIAPEK |
| | | VQLQDPYTYLGFELNGPKITNQKAVIRKDKLQTLNDFQKLLGDINWLRPYLKLTTGDLKPLFDTLKGDSDPNSH |
| | | RSLSKEALASLEKVETAIAEQFVTHINYSLPLIFLIFNTALTPTGLFWQDNPIMWIHLPASPKKVLLPYYDAIA |
| | | DLIILGRDHSKKYFGIEPSTIIQPYSKSQIDWLMQNTEMWPIACASFVGILDNHYPPNKLIQFCKLHTFVFPQI |
| | | ISKTPLNNALLVFTDGSSTGMAAYTLTDTTIKFQTNLNSAQLVELQALIAVLSAFPNQPLNIYTDSAYLAHSIP |
| | | LLETVAQIKHISETAKLFLQCQQLIYNRSIPFYIGHVRAHSGLPGPIAQGNQRADLATKIVASNINT |
| MPMV_ | 8,098 | LTAAIDILAPQQCAEPITWKSDEPVWVDQWPLTNDKLAAAQQLVQEQLEAGHITESSSPWNTPIFVIKKKSGKW |
| P07572_ | | RLLQDLRAVNATMVLMGALQPGLPSPVAPPQGYLKIIIDLKDCFFSIPLHPSDQKRFAFSLPSTNFKEPMQRFQ |
| 2mutB | | WKVLPQGMANSPTLCQKYVATAIHKVRHAWKQMYIIHYMDDILIAGKDGQQVLQCFDQLKQELTAAGLHIAPEK |
| | | VQLQDPYTYLGFELNGPKITNQKAVIRKDKLQTLNDFQKLLGDINWLRPYLKLTTGDLKPLFDTLKPDSDPNSH |
| | | RSLSKEALASLEKVETAIAEQFVTHINYSLPLIFLIFNTALTPTGLFWQDNPIMWIHLPASPKKVLLPYYDAIA |
| | | DLIILGRDHSKKYFGIEPSTIIQPYSKSQIDWLMQNTEMWPIACASFVGILDNHYPPNKLIQFCKLHTFVFPQI |
| | | ISKTPLNNALLVFTDGSSTGMAAYTLTDTTIKFQTNLNSAQLVELQALIAVLSAFPNQPLNIYTDSAYLAHSIP |
| | | LLETVAQIKHISETAKLFLQCQQLIYNRSIPFYIGHVRAHSGLPGPIAQGNQRADLATKIVASNINT |
| PERV_ | 8,099 | TLQLDDEYRLYSPLVKPDQNIQFWLEQFPQAWAETAGMGLAKQVPPQVIQLKASATPVSVRQYPLSKEAQEGIR |
| Q4VFZ2 | | PHVQRLIQQGILVPVQSPWNTPLLPVRKPGTNDYRPVQDLREVNKRVQDIHPTVPNPYNLLCALPPQRSWYTVL |
| | | DLKDAFFCLRLHPTSQPLFAFEWRDPGTGRTGQLTWTRLPQGFKNSPTIFDEALHRDLANFRIQHPQVTLLQYV |
| | | DDLLLAGATKQDCLEGTKALLLELSDLGYRASAKKAQICRREVTYLGYSLRDGQRWLTEARKKTVVQIPAPTTA |
| | | KQVREFLGTAGFCRLWIPGFATLAAPLYPLTKEKGEFSWAPEHQKAFDAIKKALLSAPALALPDVTKPFTLYVD |
| | | ERKGVARGVLTQTLGPWRRPVAYLSKKLDPVASGWPVCLKAIAAVAILVKDADKLTLGQNITVIAPHALENIVR |
| | | QPPDRWMTNARMTHYQSLLLTERVTFAPPAALNPATLLPEETDEPVTHDCHQLLIEETGVRKDLTDIPLTGEVL |
| | | TWFTDGSSYVVEGKRMAGAAVVDGTRTIWASSLPEGTSAQKAELMALTQALRLAEGKSINIYTDSRYAFATAHV |
| | | HGAIYKQRGLLTSAGREIKNKEEILSLLEALHLPKRLAIIHCPGHQKAKDPISRGNQMADRVAKQAAQGVNLL |
| PERV_ | 8,100 | TLQLDDEYRLYSPLVKPDQNIQFWLEQFPQAWAETAGMGLAKQVPPQVIQLKASATPVSVRQYPLSKEAQEGIR |
| Q4VFZ2 | | PHVQRLIQQGILVPVQSPWNTPLLPVRKPGTNDYRPVQDLREVNKRVQDIHPTVPNPYNLLCALPPQRSWYTVL |
| | | DLKDAFFCLRLHPTSQPLFAFEWRDPGTGRTGQLTWTRLPQGFKNSPTIFDEALHRDLANFRIQHPQVTLLQYV |
| | | DDLLLAGATKQDCLEGTKALLLELSDLGYRASAKKAQICRREVTYLGYSLRDGQRWLTEARKKTVVQIPAPTTA |
| | | KQVREFLGTAGFCRLWIPGFATLAAPLYPLTKEKGEFSWAPEHQKAFDAIKKALLSAPALALPDVTKPFTLYVD |
| | | ERKGVARGVLTQTLGPWRRPVAYLSKKLDPVASGWPVCLKAIAAVAILVKDADKLTLGQNITVIAPHALENIVR |
| | | QPPDRWMTNARMTHYQSLLLTERVTFAPPAALNPATLLPEETDEPVTHDCHQLLIEETGVRKDLTDIPLTGEVL |
| | | TWFTDGSSYVVEGKRMAGAAVVDGTRTIWASSLPEGTSAQKAELMALTQALRLAEGKSINIYTDSRYAFATAHV |
| | | HGAIYKQRGLLTSAGREIKNKEEILSLLEALHLPKRLAIIHCPGHQKAKDPISRGNQMADRVAKQAAQGVNLL |
| PERV_ | 8,101 | TLQLDDEYRLYSPLVKPDQNIQFWLEQFPQAWAETAGMGLAKQVPPQVIQLKASATPVSVRQYPLSKEAQEGIR |
| Q4VFZ2_ | | PHVQRLIQQGILVPVQSPWNTPLLPVRKPGTNDYRPVQDLREVNKRVQDIHPTVPNPYNLLCALPPQRSWYTVL |
| 3mut | | DLKDAFFCLRLHPTSQPLFAFEWRDPGTGRTGQLTWTRLPQGFKNSPTIFNEALHRDLANFRIQHPQVTLLQYV |
| | | DDLLLAGATKQDCLEGTKALLLELSDLGYRASAKKAQICRREVTYLGYSLRDGQRWLTEARKKTVVQIPAPTTA |
| | | KQVREFLGTAGFCRLWIPGFATLAAPLYPLTKPKGEFSWAPEHQKAFDAIKKALLSAPALALPDVTKPFTLYVD |
| | | ERKGVARGVLTQTLGPWRRPVAYLSKKLDPVASGWPVCLKAIAAVAILVKDADKLTLGQNITVIAPHALENIVR |
| | | QPPDRWMTNARMTHYQSLLLTERVTFAPPAALNPATLLPEETDEPVTHDCHQLLIEETGVRKDLTDIPLTGEVL |
| | | TWFTDGSSYVVEGKRMAGAAVVDGTRTIWASSLPEGTSAQKAELMALTQALRLAEGKSINIYTDSRYAFATAHV |
| | | HGAIYKQRGWLTSAGREIKNKEEILSLLEALHLPKRLAIIHCPGHQKAKDPISRGNQMADRVAKQAAQGVNLL |
| PERV_ | 8,102 | TLQLDDEYRLYSPLVKPDQNIQFWLEQFPQAWAETAGMGLAKQVPPQVIQLKASATPVSVRQYPLSKEAQEGIR |
| Q4VFZ2_ | | PHVQRLIQQGILVPVQSPWNTPLLPVRKPGTNDYRPVQDLREVNKRVQDIHPTVPNPYNLLCALPPQRSWYTVL |
| 3mut | | DLKDAFFCLRLHPTSQPLFAFEWRDPGTGRTGQLTWTRLPQGFKNSPTIFNEALHRDLANFRIQHPQVTLLQYV |
| | | DDLLLAGATKQDCLEGTKALLLELSDLGYRASAKKAQICRREVTYLGYSLRDGQRWLTEARKKTVVQIPAPTTA |
| | | KQVREFLGTAGFCRLWIPGFATLAAPLYPLTKPKGEFSWAPEHQKAFDAIKKALLSAPALALPDVTKPFTLYVD |
| | | ERKGVARGVLTQTLGPWRRPVAYLSKKLDPVASGWPVCLKAIAAVAILVKDADKLTLGQNITVIAPHALENIVR |
| | | QPPDRWMTNARMTHYQSLLLTERVTFAPPAALNPATLLPEETDEPVTHDCHQLLIEETGVRKDLTDIPLTGEVL |
| | | TWFTDGSSYVVEGKRMAGAAVVDGTRTIWASSLPEGTSAQKAELMALTQALRLAEGKSINIYTDSRYAFATAHV |
| | | HGAIYKQRGWLTSAGREIKNKEEILSLLEALHLPKRLAIIHCPGHQKAKDPISRGNQMADRVAKQAAQGVNLL |
| PERV_ | 8,103 | LDDEYRLYSPLVKPDQNIQFWLEQFPQAWAETAGMGLAKQVPPQVIQLKASATPVSVRQYPLSKEAQEGIRPHV |
| Q4VFZ2_ | | QRLIQQGILVPVQSPWNTPLLPVRKPGTNDYRPVQDLREVNKRVQDIHPTVPNPYNLLCALPPQRSWYTVLDLK |
| 3mutA_ | | DAFFCLRLHPTSQPLFAFEWRDPGTGRTGQLTWTRLPQGFKNSPTIFNEALHRDLANFRIQHPQVTLLQYVDDL |
| WS | | LLAGATKQDCLEGTKALLLELSDLGYRASAKKAQICRREVTYLGYSLRDGQRWLTEARKKTVVQIPAPTTAKQV |
| | | REFLGKAGFCRLFIPGFATLAAPLYPLTKPKGEFSWAPEHQKAFDAIKKALLSAPALALPDVTKPFTLYVDERK |
| | | GVARGVLTQTLGPWRRPVAYLSKKLDPVASGWPVCLKAIAAVAILVKDADKLTLGQNITVIAPHALENIVRQPP |
| | | DRWMTNARMTHYQSLLLTERVTFAPPAALNPATLLPEETDEPVTHDCHQLLIEETGVRKDLTDIPLTGEVLTWF |
| | | TDGSSYVVEGKRMAGAAVVDGTRTIWASSLPEGTSAQKAELMALTQALRLAEGKSINIYTDSRYAFATAHVHGA |
| | | IYKQRGWLTSAGREIKNKEEILSLLEALHLPKRLAIIHCPGHQKAKDPISRGNQMADRVAKQAAQGVNLLP |
| PERV_ | 8,104 | LDDEYRLYSPLVKPDQNIQFWLEQFPQAWAETAGMGLAKQVPPQVIQLKASATPVSVRQYPLSKEAQEGIRPHV |
| Q4VFZ2_ | | QRLIQQGILVPVQSPWNTPLLPVRKPGTNDYRPVQDLREVNKRVQDIHPTVPNPYNLLCALPPQRSWYTVLDLK |
| 3mutA_ | | DAFFCLRLHPTSQPLFAFEWRDPGTGRTGQLTWTRLPQGFKNSPTIFNEALHRDLANFRIQHPQVTLLQYVDDL |
| WS | | LLAGATKQDCLEGTKALLLELSDLGYRASAKKAQICRREVTYLGYSLRDGQRWLTEARKKTVVQIPAPTTAKQV |
| | | REFLGKAGFCRLFIPGFATLAAPLYPLTKPKGEFSWAPEHQKAFDAIKKALLSAPALALPDVTKPFTLYVDERK |
| | | GVARGVLTQTLGPWRRPVAYLSKKLDPVASGWPVCLKAIAAVAILVKDADKLTLGQNITVIAPHALENIVRQPP |
| | | DRWMTNARMTHYQSLLLTERVTFAPPAALNPATLLPEETDEPVTHDCHQLLIEETGVRKDLTDIPLTGEVLTWF |
| | | TDGSSYVVEGKRMAGAAVVDGTRTIWASSLPEGTSAQKAELMALTQALRLAEGKSINIYTDSRYAFATAHVHGA |
| | | IYKQRGWLTSAGREIKNKEEILSLLEALHLPKRLAIIHCPGHQKAKDPISRGNQMADRVAKQAAQGVNLLP |
| SFV1_ | 8,105 | MDPLQLLQPLEAEIKGTKLKAHWNSGATITCVPEAFLEDERPIQTMLIKTIHGEKQQDVYYLTFKVQGRKVEAE |
| P23074 | | VLASPYDYILLNPSDVPWLMKKPLQLTVLVPLHEYQERLLQQTALPKEQKELLQKLFLKYDALWQHWENQVGHR |
| | | RIKPHNIATGTLAPRPQKQYPINPKAKPSIQIVIDDLLKQGVLIQQNSTMNTPVYPVPKPDGKWRMVLDYREVN |
| | | KTIPLIAAQNQHSAGILSSIYRGKYKTTLDLTNGFWAHPITPESYWLTAFTWQGKQYCWTRLPQGFLNSPALFT |
| | | ADWVDLLKEIPNVQAYVDDIYISHDDPQEHLEQLEKIFSILLNAGYVVSLKKSEIAQREVEFLGFNITKEGRGL |
| | | TDTFKQKLLNITPPKDLKQLQSILGLLNFARNFIPNYSELVKPLYTIVANANGKFISWTEDNSNQLQHIISVLN |
| | | QADNLEERNPETRLIIKVNSSPSAGYIRYYNEGSKRPIMYVNYIFSKAEAKFTQTEKLLTTMHKGLIKAMDLAM |
| | | GQEILVYSPIVSMTKIQRTPLPERKALPVRWITWMTYLEDPRIQFHYDKSLPELQQIPNVTEDVIAKTKHPSEF |
| | | AMVFYTDGSAIKHPDVNKSHSAGMGIAQVQFIPEYKIVHQWSIPLGDHTAQLAEIAAVEFACKKALKISGPVLI |
| | | VTDSFYVAESANKELPYWKSNGFLNNKKKPLRHVSKWKSIAECLQLKPDIIIMHEKGHQQPMTTLHTEGNNLAD |
| | | KLATQGSYVVH |
| SFV1_ | 8,106 | MDPLQLLQPLEAEIKGTKLKAHWNSGATITCVPEAFLEDERPIQTMLIKTIHGEKQQDVYYLTFKVQGRKVEAE |
| P23074_ | | VLASPYDYILLNPSDVPWLMKKPLQLTVLVPLHEYQERLLQQTALPKEQKELLQKLFLKYDALWQHWENQVGHR |
| 2mut | | RIKPHNIATGTLAPRPQKQYPINPKAKPSIQIVIDDLLKQGVLIQQNSTMNTPVYPVPKPDGKWRMVLDYREVN |
| | | KTIPLIAAQNQHSAGILSSIYRGKYKTTLDLTNGFWAHPITPESYWLTAFTWQGKQYCWTRLPQGFLNSPALFN |
| | | ADWVDLLKEIPNVQAYVDDIYISHDDPQEHLEQLEKIFSILLNAGYVVSLKKSEIAQREVEFLGFNITKEGRGL |
| | | TDTFKQKLLNITPPKDLKQLQSILGLLNFARNFIPNYSELVKPLYTIVAPANGKFISWTEDNSNQLQHIISVLN |
| | | QADNLEERNPETRLIIKVNSSPSAGYIRYYNEGSKRPIMYVNYIFSKAEAKFTQTEKLLTTMHKGLIKAMDLAM |
| | | GQEILVYSPIVSMTKIQRTPLPERKALPVRWITWMTYLEDPRIQFHYDKSLPELQQIPNVTEDVIAKTKHPSEF |
| | | AMVFYTDGSAIKHPDVNKSHSAGMGIAQVQFIPEYKIVHQWSIPLGDHTAQLAEIAAVEFACKKALKISGPVLI |
| | | VTDSFYVAESANKELPYWKSNGFLNNKKKPLRHVSKWKSIAECLQLKPDIIIMHEKGHQQPMTTLHTEGNNLAD |
| | | KLATQGSYVVH |
| SFV1_ | 8,107 | MDPLQLLQPLEAEIKGTKLKAHWNSGATITCVPEAFLEDERPIQTMLIKTIHGEKQQDVYYLTFKVQGRKVEAE |
| P23074_ | | VLASPYDYILLNPSDVPWLMKKPLQLTVLVPLHEYQERLLQQTALPKEQKELLQKLFLKYDALWQHWENQVGHR |
| 2mutA | | RIKPHNIATGTLAPRPQKQYPINPKAKPSIQIVIDDLLKQGVLIQQNSTMNTPVYPVPKPDGKWRMVLDYREVN |
| | | KTIPLIAAQNQHSAGILSSIYRGKYKTTLDLTNGFWAHPITPESYWLTAFTWQGKQYCWTRLPQGFLNSPALFN |
| | | ADWVDLLKEIPNVQAYVDDIYISHDDPQEHLEQLEKIFSILLNAGYVVSLKKSEIAQREVEFLGFNITKEGRGL |
| | | TDTFKQKLLNITPPKDLKQLQSILGKLNFARNFIPNYSELVKPLYTIVAPANGKFISWTEDNSNQLQHIISVLN |
| | | QADNLEERNPETRLIIKVNSSPSAGYIRYYNEGSKRPIMYVNYIFSKAEAKFTQTEKLLTTMHKGLIKAMDLAM |
| | | GQEILVYSPIVSMTKIQRTPLPERKALPVRWITWMTYLEDPRIQFHYDKSLPELQQIPNVTEDVIAKTKHPSEF |
| | | AMVFYTDGSAIKHPDVNKSHSAGMGIAQVQFIPEYKIVHQWSIPLGDHTAQLAEIAAVEFACKKALKISGPVLI |
| | | VTDSFYVAESANKELPYWKSNGFLNNKKKPLRHVSKWKSIAECLQLKPDIIIMHEKGHQQPMTTLHTEGNNLAD |
| | | KLATQGSYVVH |
| SFV1_ | 8,108 | VPWLMKKPLQLTVLVPLHEYQERLLQQTALPKEQKELLQKLFLKYDALWQHWENQVGHRRIKPHNIATGTLAPR |
| P23074- | | PQKQYPINPKAKPSIQIVIDDLLKQGVLIQQNSTMNTPVYPVPKPDGKWRMVLDYREVNKTIPLIAAQNQHSAG |
| Pro | | ILSSIYRGKYKTTLDLTNGFWAHPITPESYWLTAFTWQGKQYCWTRLPQGFLNSPALFTADVVDLLKEIPNVQA |
| | | YVDDIYISHDDPQEHLEQLEKIFSILLNAGYVVSLKKSEIAQREVEFLGFNITKEGRGLTDTFKQKLLNITPPK |
| | | DLKQLQSILGLLNFARNFIPNYSELVKPLYTIVANANGKFISWTEDNSNQLQHIISVLNQADNLEERNPETRLI |
| | | IKVNSSPSAGYIRYYNEGSKRPIMYVNYIFSKAEAKFTQTEKLLTTMHKGLIKAMDLAMGQEILVYSPIVSMTK |
| | | IQRTPLPERKALPVRWITWMTYLEDPRIQFHYDKSLPELQQIPNVTEDVIAKTKHPSEFAMVFYTDGSAIKHPD |
| | | VNKSHSAGMGIAQVQFIPEYKIVHQWSIPLGDHTAQLAEIAAVEFACKKALKISGPVLIVTDSFYVAESANKEL |
| | | PYWKSNGFLNNKKKPLRHVSKWKSIAECLQLKPDIIIMHEKGHQQPMTTLHTEGNNLADKLATQGSYVVH |
| SFV1_ | 8,109 | VPWLMKKPLQLTVLVPLHEYQERLLQQTALPKEQKELLQKLFLKYDALWQHWENQVGHRRIKPHNIATGTLAPR |
| P23074- | | PQKQYPINPKAKPSIQIVIDDLLKQGVLIQQNSTMNTPVYPVPKPDGKWRMVLDYREVNKTIPLIAAQNQHSAG |
| Pro_ | | ILSSIYRGKYKTTLDLTNGFWAHPITPESYWLTAFTWQGKQYCWTRLPQGFLNSPALFNADVVDLLKEIPNVQA |
| 2mut | | YVDDIYISHDDPQEHLEQLEKIFSILLNAGYVVSLKKSEIAQREVEFLGFNITKEGRGLTDTFKQKLLNITPPK |
| | | DLKQLQSILGLLNFARNFIPNYSELVKPLYTIVAPANGKFISWTEDNSNQLQHIISVLNQADNLEERNPETRLI |
| | | IKVNSSPSAGYIRYYNEGSKRPIMYVNYIFSKAEAKFTQTEKLLTTMHKGLIKAMDLAMGQEILVYSPIVSMTK |
| | | IQRTPLPERKALPVRWITWMTYLEDPRIQFHYDKSLPELQQIPNVTEDVIAKTKHPSEFAMVFYTDGSAIKHPD |
| | | VNKSHSAGMGIAQVQFIPEYKIVHQWSIPLGDHTAQLAEIAAVEFACKKALKISGPVLIVTDSFYVAESANKEL |
| | | PYWKSNGFLNNKKKPLRHVSKWKSIAECLQLKPDIIIMHEKGHQQPMTTLHTEGNNLADKLATQGSYVVH |
| SFV1_ | 8,110 | VPWLMKKPLQLTVLVPLHEYQERLLQQTALPKEQKELLQKLFLKYDALWQHWENQVGHRRIKPHNIATGTLAPR |
| P23074 | | PQKQYPINPKAKPSIQIVIDDLLKQGVLIQQNSTMNTPVYPVPKPDGKWRMVLDYREVNKTIPLIAAQNQHSAG |
| Pro_2m | | ILSSIYRGKYKTTLDLTNGFWAHPITPESYWLTAFTWQGKQYCWTRLPQGFLNSPALFNADVVDLLKEIPNVQA |
| utA | | YVDDIYISHDDPQEHLEQLEKIFSILLNAGYVVSLKKSEIAQREVEFLGFNITKEGRGLTDTFKQKLLNITPPK |
| | | DLKQLQSILGKLNFARNFIPNYSELVKPLYTIVAPANGKFISWTEDNSNQLQHIISVLNQADNLEERNPETRLI |
| | | IKVNSSPSAGYIRYYNEGSKRPIMYVNYIFSKAEAKFTQTEKLLTTMHKGLIKAMDLAMGQEILVYSPIVSMTK |
| | | IQRTPLPERKALPVRWITWMTYLEDPRIQFHYDKSLPELQQIPNVTEDVIAKTKHPSEFAMVFYTDGSAIKHPD |
| | | VNKSHSAGMGIAQVQFIPEYKIVHQWSIPLGDHTAQLAEIAAVEFACKKALKISGPVLIVTDSFYVAESANKEL |
| | | PYWKSNGFLNNKKKPLRHVSKWKSIAECLQLKPDIIIMHEKGHQQPMTTLHTEGNNLADKLATQGSYVVH |
| SFV3L_ | 8,111 | MDPLQLLQPLEAEIKGTKLKAHWNSGATITCVPQAFLEEEVPIKNIWIKTIHGEKEQPVYYLTFKIQGRKVEAE |
| P27401 | | VISSPYDYILVSPSDIPWLMKKPLQLTTLVPLQEYEERLLKQTMLTGSYKEKLQSLFLKYDALWQHWENQVGHR |
| | | RIKPHHIATGTVNPRPQKQYPINPKAKASIQTVINDLLKQGVLIQQNSIMNTPVYPVPKPDGKWRMVLDYREVN |
| | | KTIPLIAAQNQHSAGILSSIFRGKYKTTLDLSNGFWAHSITPESYWLTAFTWLGQQYCWTRLPQGFLNSPALFT |
| | | ADVVDLLKEVPNVQVYVDDIYISHDDPREHLEQLEKVFSLLLNAGYVVSLKKSEIAQHEVEFLGFNITKEGRGL |
| | | TETFKQKLLNITPPRDLKQLQSILGLLNFARNFIPNFSELVKPLYNIIATANGKYITWTTDNSQQLQNIISMLN |
| | | SAENLEERNPEVRLIMKVNTSPSAGYIRFYNEFAKRPIMYLNYVYTKAEVKFTNTEKLLTTIHKGLIKALDLGM |
| | | GQEILVYSPIVSMTKIQKTPLPERKALPIRWITWMSYLEDPRIQFHYDKTLPELQQVPTVTDDIIAKIKHPSEF |
| | | SMVFYTDGSAIKHPNVNKSHNAGMGIAQVQFKPEFTVINTWSIPLGDHTAQLAEVAAVEFACKKALKIDGPVLI |
| | | VTDSFYVAESVNKELPYWQSNGFFNNKKKPLKHVSKWKSIADCIQLKPDIIIIHEKGHQPTASTFHTEGNNLAD |
| | | KLATQGSYVVN |
| SFV3L_ | 8,112 | MDPLQLLQPLEAEIKGTKLKAHWNSGATITCVPQAFLEEEVPIKNIWIKTIHGEKEQPVYYLTFKIQGRKVEAE |
| P27401_ | | VISSPYDYILVSPSDIPWLMKKPLQLTTLVPLQEYEERLLKQTMLTGSYKEKLQSLFLKYDALWQHWENQVGHR |
| 2mut | | RIKPHHIATGTVNPRPQKQYPINPKAKASIQTVINDLLKQGVLIQQNSIMNTPVYPVPKPDGKWRMVLDYREVN |
| | | KTIPLIAAQNQHSAGILSSIFRGKYKTTLDLSNGFWAHSITPESYWLTAFTWLGQQYCWTRLPQGFLNSPALFN |
| | | ADVVDLLKEVPNVQVYVDDIYISHDDPREHLEQLEKVFSLLLNAGYVVSLKKSEIAQHEVEFLGFNITKEGRGL |
| | | TETFKQKLLNITPPRDLKQLQSILGLLNFARNFIPNFSELVKPLYNIIATAPGKYITWTTDNSQQLQNIISMLN |
| | | SAENLEERNPEVRLIMKVNTSPSAGYIRFYNEFAKRPIMYLNYVYTKAEVKFTNTEKLLTTIHKGLIKALDLGM |
| | | GQEILVYSPIVSMTKIQKTPLPERKALPIRWITWMSYLEDPRIQFHYDKTLPELQQVPTVTDDIIAKIKHPSEF |
| | | SMVFYTDGSAIKHPNVNKSHNAGMGIAQVQFKPEFTVINTWSIPLGDHTAQLAEVAAVEFACKKALKIDGPVLI |
| | | VTDSFYVAESVNKELPYWQSNGFFNNKKKPLKHVSKWKSIADCIQLKPDIIIIHEKGHQPTASTFHTEGNNLAD |
| | | KLATQGSYVVN |
| SFV3L_ | 8,113 | MDPLQLLQPLEAEIKGTKLKAHWNSGATITCVPQAFLEEEVPIKNIWIKTIHGEKEQPVYYLTFKIQGRKVEAE |
| P27401_ | | VISSPYDYILVSPSDIPWLMKKPLQLTTLVPLQEYEERLLKQTMLTGSYKEKLQSLFLKYDALWQHWENQVGHR |
| 2mutA | | RIKPHHIATGTVNPRPQKQYPINPKAKASIQTVINDLLKQGVLIQQNSIMNTPVYPVPKPDGKWRMVLDYREVN |
| | | KTIPLIAAQNQHSAGILSSIFRGKYKTTLDLSNGFWAHSITPESYWLTAFTWLGQQYCWTRLPQGFLNSPALFN |
| | | ADVVDLLKEVPNVQVYVDDIYISHDDPREHLEQLEKVFSLLLNAGYVVSLKKSEIAQHEVEFLGFNITKEGRGL |
| | | TETFKQKLLNITPPRDLKQLQSILGKLNFARNFIPNFSELVKPLYNIIATAPGKYITWTTDNSQQLQNIISMLN |
| | | SAENLEERNPEVRLIMKVNTSPSAGYIRFYNEFAKRPIMYLNYVYTKAEVKFTNTEKLLTTIHKGLIKALDLGM |
| | | GQEILVYSPIVSMTKIQKTPLPERKALPIRWITWMSYLEDPRIQFHYDKTLPELQQVPTVTDDIIAKIKHPSEF |
| | | SMVFYTDGSAIKHPNVNKSHNAGMGIAQVQFKPEFTVINTWSIPLGDHTAQLAEVAAVEFACKKALKIDGPVLI |
| | | VTDSFYVAESVNKELPYWQSNGFFNNKKKPLKHVSKWKSIADCIQLKPDIIIIHEKGHQPTASTFHTEGNNLAD |
| | | KLATQGSYVVN |
| SFV3L_ | 8,114 | IPWLMKKPLQLTTLVPLQEYEERLLKQTMLTGSYKEKLQSLFLKYDALWQHWENQVGHRRIKPHHIATGTVNPR |
| P27401- | | PQKQYPINPKAKASIQTVINDLLKQGVLIQQNSIMNTPVYPVPKPDGKWRMVLDYREVNKTIPLIAAQNQHSAG |
| Pro | | ILSSIFRGKYKTTLDLSNGFWAHSITPESYWLTAFTWLGQQYCWTRLPQGFLNSPALFTADVVDLLKEVPNVQV |
| | | YVDDIYISHDDPREHLEQLEKVFSLLLNAGYVVSLKKSEIAQHEVEFLGFNITKEGRGLTETFKQKLLNITPPR |
| | | DLKQLQSILGLLNFARNFIPNFSELVKPLYNIIATANGKYITWTTDNSQQLQNIISMLNSAENLEERNPEVRLI |
| | | MKVNTSPSAGYIRFYNEFAKRPIMYLNYVYTKAEVKFTNTEKLLTTIHKGLIKALDLGMGQEILVYSPIVSMTK |
| | | IQKTPLPERKALPIRWITWMSYLEDPRIQFHYDKTLPELQQVPTVTDDIIAKIKHPSEFSMVFYTDGSAIKHPN |
| | | VNKSHNAGMGIAQVQFKPEFTVINTWSIPLGDHTAQLAEVAAVEFACKKALKIDGPVLIVTDSFYVAESVNKEL |
| | | PYWQSNGFFNNKKKPLKHVSKWKSIADCIQLKPDIIIIHEKGHQPTASTFHTEGNNLADKLATQGSYVVN |
| SFV3L_ | 8,115 | IPWLMKKPLOLTTLVPLQEYEERLLKQTMLTGSYKEKLQSLFLKYDALWQHWENQVGHRRIKPHHIATGTVNPR |
| P27401- | | PQKQYPINPKAKASIQTVINDLLKQGVLIQQNSIMNTPVYPVPKPDGKWRMVLDYREVNKTIPLIAAQNQHSAG |
| Pro_ | | ILSSIFRGKYKTTLDLSNGFWAHSITPESYWLTAFTWLGQQYCWTRLPQGFLNSPALFNADVVDLLKEVPNVQV |
| 2mut | | YVDDIYISHDDPREHLEQLEKVFSLLLNAGYVVSLKKSEIAQHEVEFLGFNITKEGRGLTETFKQKLLNITPPR |
| | | DLKQLQSILGLLNFARNFIPNFSELVKPLYNIIATAPGKYITWTTDNSQQLQNIISMLNSAENLEERNPEVRLI |
| | | MKVNTSPSAGYIRFYNEFAKRPIMYLNYVYTKAEVKFTNTEKLLTTIHKGLIKALDLGMGQEILVYSPIVSMTK |
| | | IQKTPLPERKALPIRWITWMSYLEDPRIQFHYDKTLPELQQVPTVTDDIIAKIKHPSEFSMVFYTDGSAIKHPN |
| | | VNKSHNAGMGIAQVQFKPEFTVINTWSIPLGDHTAQLAEVAAVEFACKKALKIDGPVLIVTDSFYVAESVNKEL |
| | | PYWQSNGFFNNKKKPLKHVSKWKSIADCIQLKPDIIIIHEKGHQPTASTFHTEGNNLADKLATQGSYVVN |
| SFV3L_ | 8,116 | IPWLMKKPLQLTTLVPLQEYEERLLKQTMLTGSYKEKLQSLFLKYDALWQHWENQVGHRRIKPHHIATGTVNPR |
| P27401- | | PQKQYPINPKAKASIQTVINDLLKQGVLIQQNSIMNTPVYPVPKPDGKWRMVLDYREVNKTIPLIAAQNQHSAG |
| Pro_ | | ILSSIFRGKYKTTLDLSNGFWAHSITPESYWLTAFTWLGQQYCWTRLPQGFLNSPALFNADVVDLLKEVPNVQV |
| 2mutA | | YVDDIYISHDDPREHLEQLEKVFSLLLNAGYVVSLKKSEIAQHEVEFLGFNITKEGRGLTETFKQKLLNITPPR |
| | | DLKQLQSILGKLNFARNFIPNFSELVKPLYNIIATAPGKYITWTTDNSQQLQNIISMLNSAENLEERNPEVRLI |
| | | MKVNTSPSAGYIRFYNEFAKRPIMYLNYVYTKAEVKFTNTEKLLTTIHKGLIKALDLGMGQEILVYSPIVSMTK |
| | | IQKTPLPERKALPIRWITWMSYLEDPRIQFHYDKTLPELQQVPTVTDDIIAKIKHPSEFSMVFYTDGSAIKHPN |
| | | VNKSHNAGMGIAQVQFKPEFTVINTWSIPLGDHTAQLAEVAAVEFACKKALKIDGPVLIVTDSFYVAESVNKEL |
| | | PYWQSNGFFNNKKKPLKHVSKWKSIADCIQLKPDIIIIHEKGHQPTASTFHTEGNNLADKLATQGSYVVN |
| SFVCP_ | 8,117 | MNPLQLLQPLPAEVKGTKLLAHWNSGATITCIPESFLEDEQPIKQTLIKTIHGEKQQNVYYLTFKVKGRKVEAE |
| Q87040 | | VIASPYEYILLSPTDVPWLTQQPLQLTILVPLQEYQDRINKTALPEEQKQQLKALFTKYDNLWQHWENQVGHRK |
| | | IRPHNIATGDYPPRPQKQYPINPKAKPSIQIVIDDLLKQGVLTPQNSTMNTPVYPVPKPDGRWRMVLDYREVNK |
| | | TIPLTAAQNQHSAGILATIVRQKYKTTLDLANGFWAHPITPDSYWLTAFTWQGKQYCWTRLPQGFLNSPALFTA |
| | | DAVDLLKEVPNVQVYVDDIYLSHDNPHEHIQQLEKVFQILLQAGYVVSLKKSEIGQRTVEFLGFNITKEGRGLT |
| | | DTFKTKLLNVTPPKDLKQLQSILGLLNFARNFIPNFAELVQTLYNLIASSKGKYIEWTEDNTKQLNKVIEALNT |
| | | ASNLEERLPDQRLVIKVNTSPSAGYVRYYNESGKKPIMYLNYVFSKAELKFSMLEKLLTTMHKALIKAMDLAMG |
| | | QEILVYSPIVSMTKIQKTPLPERKALPIRWITWMTYLEDPRIQFHYDKTLPELKHIPDVYTSSIPPLKHPSQYE |
| | | GVFCTDGSAIKSPDPTKSNNAGMGIVHAIYNPEYKILNQWSIPLGHHTAQMAEIAAVEFACKKALKVPGPVLVI |
| | | TDSFYVAESANKELPYWKSNGFVNNKKEPLKHISKWKSIAECLSIKPDITIQHEKGHQPINTSIHTEGNALADK |
| | | LATQGSYVVN |
| SFVCP_ | 8,118 | MNPLQLLQPLPAEVKGTKLLAHWNSGATITCIPESFLEDEQPIKQTLIKTIHGEKQQNVYYLTFKVKGRKVEAE |
| Q87040_ | | VIASPYEYILLSPTDVPWLTQQPLQLTILVPLQEYQDRINKTALPEEQKQQLKALFTKYDNLWQHWENQVGHRK |
| 2mut | | IRPHNIATGDYPPRPQKQYPINPKAKPSIQIVIDDLLKQGVLTPQNSTMNTPVYPVPKPDGRWRMVLDYREVNK |
| | | TIPLTAAQNQHSAGILATIVRQKYKTTLDLANGFWAHPITPDSYWLTAFTWQGKQYCWTRLPQGFLNSPALFNA |
| | | DAVDLLKEVPNVQVYVDDIYLSHDNPHEHIQQLEKVFQILLQAGYVVSLKKSEIGQRTVEFLGFNITKEGRGLT |
| | | DTFKTKLLNVTPPKDLKQLQSILGLLNFARNFIPNFAELVQTLYNLIASSPGKYIEWTEDNTKQLNKVIEALNT |
| | | ASNLEERLPDQRLVIKVNTSPSAGYVRYYNESGKKPIMYLNYVFSKAELKFSMLEKLLTTMHKALIKAMDLAMG |
| | | QEILVYSPIVSMTKIQKTPLPERKALPIRWITWMTYLEDPRIQFHYDKTLPELKHIPDVYTSSIPPLKHPSQYE |
| | | GVFCTDGSAIKSPDPTKSNNAGMGIVHAIYNPEYKILNQWSIPLGHHTAQMAEIAAVEFACKKALKVPGPVLVI |
| | | TDSFYVAESANKELPYWKSNGFVNNKKEPLKHISKWKSIAECLSIKPDITIQHEKGHQPINTSIHTEGNALADK |
| | | LATQGSYVVN |
| SFVCP_ | 8,119 | MNPLQLLQPLPAEVKGTKLLAHWNSGATITCIPESFLEDEQPIKQTLIKTIHGEKQQNVYYLTFKVKGRKVEAE |
| Q87040_ | | VIASPYEYILLSPTDVPWLTQQPLQLTILVPLQEYQDRINKTALPEEQKQQLKALFTKYDNLWQHWENQVGHRK |
| 2mutA | | IRPHNIATGDYPPRPQKQYPINPKAKPSIQIVIDDLLKQGVLTPQNSTMNTPVYPVPKPDGRWRMVLDYREVNK |
| | | TIPLTAAQNQHSAGILATIVRQKYKTTLDLANGFWAHPITPDSYWLTAFTWQGKQYCWTRLPQGFLNSPALFNA |
| | | DAVDLLKEVPNVQVYVDDIYLSHDNPHEHIQQLEKVFQILLQAGYVVSLKKSEIGQRTVEFLGFNITKEGRGLT |
| | | DTFKTKLLNVTPPKDLKQLQSILGKLNFARNFIPNFAELVQTLYNLIASSPGKYIEWTEDNTKQLNKVIEALNT |
| | | ASNLEERLPDQRLVIKVNTSPSAGYVRYYNESGKKPIMYLNYVFSKAELKFSMLEKLLTTMHKALIKAMDLAMG |
| | | QEILVYSPIVSMTKIQKTPLPERKALPIRWITWMTYLEDPRIQFHYDKTLPELKHIPDVYTSSIPPLKHPSQYE |
| | | GVFCTDGSAIKSPDPTKSNNAGMGIVHAIYNPEYKILNQWSIPLGHHTAQMAEIAAVEFACKKALKVPGPVLVI |
| | | TDSFYVAESANKELPYWKSNGFVNNKKEPLKHISKWKSIAECLSIKPDITIQHEKGHQPINTSIHTEGNALADK |
| | | LATQGSYVVN |
| SFVCP_ | 8,120 | VPWLTQQPLQLTILVPLQEYQDRILNKTALPEEQKQQLKALFTKYDNLWQHWENQVGHRKIRPHNIATGDYPPR |
| Q87040- | | PQKQYPINPKAKPSIQIVIDDLLKQGVLTPQNSTMNTPVYPVPKPDGRWRMVLDYREVNKTIPLTAAQNQHSAG |
| Pro | | ILATIVRQKYKTTLDLANGFWAHPITPDSYWLTAFTWQGKQYCWTRLPQGFLNSPALFTADAVDLLKEVPNVQV |
| | | YVDDIYLSHDNPHEHIQQLEKVFQILLQAGYVVSLKKSEIGQRTVEFLGFNITKEGRGLTDTFKTKLLNVTPPK |
| | | DLKQLQSILGLLNFARNFIPNFAELVQTLYNLIASSKGKYIEWTEDNTKQLNKVIEALNTASNLEERLPDQRLV |
| | | IKVNTSPSAGYVRYYNESGKKPIMYLNYVFSKAELKFSMLEKLLTTMHKALIKAMDLAMGQEILVYSPIVSMTK |
| | | IQKTPLPERKALPIRWITWMTYLEDPRIQFHYDKTLPELKHIPDVYTSSIPPLKHPSQYEGVFCTDGSAIKSPD |
| | | PTKSNNAGMGIVHAIYNPEYKILNQWSIPLGHHTAQMAEIAAVEFACKKALKVPGPVLVITDSFYVAESANKEL |
| | | PYWKSNGFVNNKKEPLKHISKWKSIAECLSIKPDITIQHEKGHQPINTSIHTEGNALADKLATQGSYVVN |
| SFVCP_ | 8,121 | VPWLTQQPLQLTILVPLQEYQDRILNKTALPEEQKQQLKALFTKYDNLWQHWENQVGHRKIRPHNIATGDYPPR |
| Q87040- | | PQKQYPINPKAKPSIQIVIDDLLKQGVLTPQNSTMNTPVYPVPKPDGRWRMVLDYREVNKTIPLTAAQNQHSAG |
| Pro_ | | ILATIVRQKYKTTLDLANGFWAHPITPDSYWLTAFTWQGKQYCWTRLPQGFLNSPALFNADAVDLLKEVPNVQV |
| 2mut | | YVDDIYLSHDNPHEHIQQLEKVFQILLQAGYVVSLKKSEIGQRTVEFLGFNITKEGRGLTDTFKTKLLNVTPPK |
| | | DLKQLQSILGLLNFARNFIPNFAELVQTLYNLIASSPGKYIEWTEDNTKQLNKVIEALNTASNLEERLPDQRLV |
| | | IKVNTSPSAGYVRYYNESGKKPIMYLNYVFSKAELKFSMLEKLLTTMHKALIKAMDLAMGQEILVYSPIVSMTK |
| | | IQKTPLPERKALPIRWITWMTYLEDPRIQFHYDKTLPELKHIPDVYTSSIPPLKHPSQYEGVFCTDGSAIKSPD |
| | | PTKSNNAGMGIVHAIYNPEYKILNQWSIPLGHHTAQMAEIAAVEFACKKALKVPGPVLVITDSFYVAESANKEL |
| | | PYWKSNGFVNNKKEPLKHISKWKSIAECLSIKPDITIQHEKGHQPINTSIHTEGNALADKLATQGSYVVN |
| SFVCP_ | 8,122 | VPWLTQQPLOLTILVPLQEYQDRINKTALPEEQKQQLKALFTKYDNLWQHWENQVGHRKIRPHNIATGDYPPRP |
| Q87040- | | QKQYPINPKAKPSIQIVIDDLLKQGVLTPQNSTMNTPVYPVPKPDGRWRMVLDYREVNKTIPLTAAQNQHSAGI |
| Pro_ | | LATIVRQKYKTTLDLANGFWAHPITPDSYWLTAFTWQGKQYCWTRLPQGFLNSPALFNADAVDLLKEVPNVQVY |
| 2mutA | | VDDIYLSHDNPHEHIQQLEKVFQILLQAGYVVSLKKSEIGQRTVEFLGFNITKEGRGLTDTFKTKLLNVTPPKD |
| | | LKQLQSILGKLNFARNFIPNFAELVQTLYNLIASSPGKYIEWTEDNTKQLNKVIEALNTASNLEERLPDQRLVI |
| | | KVNTSPSAGYVRYYNESGKKPIMYLNYVFSKAELKFSMLEKLLTTMHKALIKAMDLAMGQEILVYSPIVSMTKI |
| | | QKTPLPERKALPIRWITWMTYLEDPRIQFHYDKTLPELKHIPDVYTSSIPPLKHPSQYEGVFCTDGSAIKSPDP |
| | | TKSNNAGMGIVHAIYNPEYKILNQWSIPLGHHTAQMAEIAAVEFACKKALKVPGPVLVITDSFYVAESANKELP |
| | | YWKSNGFVNNKKEPLKHISKWKSIAECLSIKPDITIQHEKGHQPINTSIHTEGNALADKLATQGSYVVN |
| SMRVH_ | 8,123 | PRSRAIDIPVPHADKISWKITDPVWVDQWPLTYEKTLAAIALVQEQLAAGHIEPTNSPWNTPIFIIKKKSGSWR |
| P03364 | | LLQDLRAVNKVMVPMGALQPGLPSPVAIPLNYHKIVIDLKDCFFTIPLHPEDRPYFAFSVPQINFQSPMPRYQW |
| | | KVLPQGMANSPTLCQKFVAAAIAPVRSQWPEAYILHYMDDILLACDSAEAAKACYAHIISCLTSYGLKIAPDKV |
| | | QVSEPFSYLGFELHHQQVFTPRVCLKTDHLKTLNDFQKLLGDIQWLRPYLKLPTSALVPLNNILKGDPNPLSVR |
| | | ALTPEAKQSLALINKAIQNQSVQQISYNLPLVLLLLPTPHTPTAVFWQPNGTDPTKNGSPLLWLHLPASPSKVL |
| | | LTYPSLLAMLIIKGRYTGRQLFGRDPHSIIIPYTQDQLTWLLQTSDEWAIALSSFTGDIDNHYPSDPVIQFAKL |
| | | HQFIFPKITKCAPIPQATLVFTDGSSNGIAAYVIDNQPISIKSPYLSAQLVELYAILQVFTVLAHQPFNLYTDS |
| | | AYIAQSVPLLETVPFIKSSTNATPLFSKLQQLILNRQHPFFIGHLRAHLNLPGPLAEGNALADAATQIFPIISD |
| SMRVH_ | 8,124 | PRSRAIDIPVPHADKISWKITDPVWVDQWPLTYEKTLAAIALVQEQLAAGHIEPTNSPWNTPIFIIKKKSGSWR |
| P03364_ | | LLQDLRAVNKVMVPMGALQPGLPSPVAIPLNYHKIVIDLKDCFFTIPLHPEDRPYFAFSVPQINFQSPMPRYQW |
| 2mut | | KVLPQGMANSPTLCQKFVAAAIAPVRSQWPEAYILHYMDDILLACDSAEAAKACYAHIISCLTSYGLKIAPDKV |
| | | QVSEPFSYLGFELHHQQVFTPRVCLKTDHLKTLNDFQKLLGDIQWLRPYLKLPTSALVPLNNILKPDPNPLSVR |
| | | ALTPEAKQSLALINKAIQNQSVQQISYNLPLVLLLLPTPHTPTAVFWQPNGTDPTKNGSPLLWLHLPASPSKVL |
| | | LTYPSLLAMLIIKGRYTGRQLFGRDPHSIIIPYTQDQLTWLLQTSDEWAIALSSFTGDIDNHYPSDPVIQFAKL |
| | | HQFIFPKITKCAPIPQATLVFTDGSSNGIAAYVIDNQPISIKSPYLSAQLVELYAILQVFTVLAHQPFNLYTDS |
| | | AYIAQSVPLLETVPFIKSSTNATPLFSKLQQLILNRQHPFFIGHLRAHLNLPGPLAEGNALADAATQIFPIISD |
| SMRVH_ | 8,125 | PRSRAIDIPVPHADKISWKITDPVWVDQWPLTYEKTLAAIALVQEQLAAGHIEPTNSPWNTPIFIIKKKSGSWR |
| P03364_ | | LLQDLRAVNKVMVPMGALQPGLPSPVAPPLNYHKIVIDLKDCFFTIPLHPEDRPYFAFSVPQINFQSPMPRYQW |
| 2mutB | | KVLPQGMANSPTLCQKFVAAAIAPVRSQWPEAYILHYMDDILLACDSAEAAKACYAHIISCLTSYGLKIAPDKV |
| | | QVSEPFSYLGFELHHQQVFTPRVCLKTDHLKTLNDFQKLLGDIQWLRPYLKLPTSALVPLNNILKPDPNPLSVR |
| | | ALTPEAKQSLALINKAIQNQSVQQISYNLPLVLLLLPTPHTPTAVFWQPNGTDPTKNGSPLLWLHLPASPSKVL |
| | | LTYPSLLAMLIIKGRYTGRQLFGRDPHSIIIPYTQDQLTWLLQTSDEWAIALSSFTGDIDNHYPSDPVIQFAKL |
| | | HQFIFPKITKCAPIPQATLVFTDGSSNGIAAYVIDNQPISIKSPYLSAQLVELYAILQVFTVLAHQPFNLYTDS |
| | | AYIAQSVPLLETVPFIKSSTNATPLFSKLQQLILNRQHPFFIGHLRAHLNLPGPLAEGNALADAATQIFPIISD |
| SRV2_ | 8,126 | LATAVDILAPQRYADPITWKSDEPVWVDQWPLTQEKLAAAQQLVQEQLQAGHIIESNSPWNTPIFVIKKKSGKW |
| P51517 | | RLLQDLRAVNATMVLMGALQPGLPSPVAIPQGYFKIVIDLKDCFFTIPLQPVDQKRFAFSLPSTNFKQPMKRYQ |
| | | WKVLPQGMANSPTLCQKYVAAAIEPVRKSWAQMYIIHYMDDILIAGKLGEQVLQCFAQLKQALTTTGLQIAPEK |
| | | VQLQDPYTYLGFQINGPKITNQKAVIRRDKLQTLNDFQKLLGDINWLRPYLHLTTGDLKPLFDILKGDSNPNSP |
| | | RSLSEAALASLQKVETAIAEQFVTQIDYTQPLTFLIFNTTLTPTGLFWQNNPVMWVHLPASPKKVLLPYYDAIA |
| | | DLIILGRDNSKKYFGLEPSTIIQPYSKSQIHWLMQNTETWPIACASYAGNIDNHYPPNKLIQFCKLHAVVFPRI |
| | | ISKTPLDNALLVFTDGSSTGIAAYTFEKTTVRFKTSHTSAQLVELQALIAVLSAFPHRALNVYTDSAYLAHSIP |
| | | LLETVSHIKHISDTAKFFLQCQQLIYNRSIPFYLGHIRAHSGLPGPLSQGNHITDLATKVVATTLTT |
| SRV2_ | 8,127 | LATAVDILAPQRYADPITWKSDEPVWVDQWPLTQEKLAAAQQLVQEQLQAGHIIESNSPWNTPIFVIKKKSGKW |
| P51517_ | | RLLQDLRAVNATMVLMGALQPGLPSPVAPPQGYFKIVIDLKDCFFTIPLQPVDQKRFAFSLPSTNFKQPMKRYQ |
| 2mutB | | WKVLPQGMANSPTLCQKYVAAAIEPVRKSWAQMYIIHYMDDILIAGKLGEQVLQCFAQLKQALTTTGLQIAPEK |
| | | VQLQDPYTYLGFQINGPKITNQKAVIRRDKLQTLNDFQKLLGDINWLRPYLHLTTGDLKPLFDILKGDSNPNSP |
| | | RSLSEAALASLQKVETAIAEQFVTQIDYTQPLTFLIFNTTLTPTGLFWQNNPVMWVHLPASPKKVLLPYYDAIA |
| | | DLIILGRDNSKKYFGLEPSTIIQPYSKSQIHWLMQNTETWPIACASYAGNIDNHYPPNKLIQFCKLHAVVFPRI |
| | | ISKTPLDNALLVFTDGSSTGIAAYTFEKTTVRFKTSHTSAQLVELQALIAVLSAFPHRALNVYTDSAYLAHSIP |
| | | LLETVSHIKHISDTAKFFLQCQQLIYNRSIPFYLGHIRAHSGLPGPLSQGNHITDLATKVVATTLTT |
| WDSV_ | 8,128 | SCQTKNTLNIDEYLLQFPDQLWASLPTDIGRMLVPPITIKIKDNASLPSIRQYPLPKDKTEGLRPLISSLENQG |
| 092815 | | ILIKCHSPCNTPIFPIKKAGRDEYRMIHDLRAINNIVAPLTAVVASPTTVLSNLAPSLHWFTVIDLSNAFFSVP |
| | | IHKDSQYLFAFTFEGHQYTWTVLPQGFIHSPTLFSQALYQSLHKIKFKISSEICIYMDDVLIASKDRDTNLKDT |
| | | AVMLQHLASEGHKVSKKKLQLCQQEVVYLGQLLTPEGRKILPDRKVTVSQFQQPTTIRQIRAFLGLVGYCRHWI |
| | | PEFSIHSKFLEKQLKKDTAEPFQLDDQQVEAFNKLKHAITTAPVLVVPDPAKPFQLYTSHSEHASIAVLTQKHA |
| | | GRTRPIAFLSSKFDAIESGLPPCLKACASIHRSLTQADSFILGAPLIIYTTHAICTLLQRDRSQLVTASRFSKW |
| | | EADLLRPELTFVACSAVSPAHLYMQSCENNIPPHDCVLLTHTISRPRPDLSDLPIPDPDMTLFSDGSYTTGRGG |
| | | AAVVMHRPVTDDFIIIHQQPGGASAQTAELLALAAACHLATDKTVNIYTDSRYAYGVVHDFGHLWMHRGFVTSA |
| | | GTPIKNHKEIEYLLKQIMKPKQVSVIKIEAHTKGVSMEVRGNAAADEAAKNAVFLVQR |
| WDSV_ | 8,129 | SCQTKNTLNIDEYLLQFPDQLWASLPTDIGRMLVPPITIKIKDNASLPSIRQYPLPKDKTEGLRPLISSLENQG |
| 092815_ | | ILIKCHSPCNTPIFPIKKAGRDEYRMIHDLRAINNIVAPLTAVVASPTTVLSNLAPSLHWFTVIDLSNAFFSVP |
| 2mut | | IHKDSQYLFAFTFEGHQYTWTVLPQGFIHSPTLFNQALYQSLHKIKFKISSEICIYMDDVLIASKDRDTNLKDT |
| | | AVMLQHLASEGHKVSKKKLQLCQQEVVYLGQLLTPEGRKILPDRKVTVSQFQQPTTIRQIRAFLGLVGYCRHWI |
| | | PEFSIHSKFLEKQLKPDTAEPFQLDDQQVEAFNKLKHAITTAPVLVVPDPAKPFQLYTSHSEHASIAVLTQKHA |
| | | GRTRPIAFLSSKFDAIESGLPPCLKACASIHRSLTQADSFILGAPLIIYTTHAICTLLQRDRSQLVTASRFSKW |
| | | EADLLRPELTFVACSAVSPAHLYMQSCENNIPPHDCVLLTHTISRPRPDLSDLPIPDPDMTLFSDGSYTTGRGG |
| | | AAVVMHRPVTDDFIIIHQQPGGASAQTAELLALAAACHLATDKTVNIYTDSRYAYGVVHDFGHLWMHRGFVTSA |
| | | GTPIKNHKEIEYLLKQIMKPKQVSVIKIEAHTKGVSMEVRGNAAADEAAKNAVFLVQR |
| WDSV_ | 8,130 | SCQTKNTLNIDEYLLQFPDQLWASLPTDIGRMLVPPITIKIKDNASLPSIRQYPLPKDKTEGLRPLISSLENQG |
| 092815_ | | ILIKCHSPCNTPIFPIKKAGRDEYRMIHDLRAINNIVAPLTAVVASPTTVLSNLAPSLHWFTVIDLSNAFFSVP |
| 2mutA | | IHKDSQYLFAFTFEGHQYTWTVLPQGFIHSPTLFNQALYQSLHKIKFKISSEICIYMDDVLIASKDRDTNLKDT |
| | | AVMLQHLASEGHKVSKKKLQLCQQEVVYLGQLLTPEGRKILPDRKVTVSQFQQPTTIRQIRAFLGKVGYCRHFI |
| | | PEFSIHSKFLEKQLKPDTAEPFQLDDQQVEAFNKLKHAITTAPVLVVPDPAKPFQLYTSHSEHASIAVLTQKHA |
| | | GRTRPIAFLSSKFDAIESGLPPCLKACASIHRSLTQADSFILGAPLIIYTTHAICTLLQRDRSQLVTASRFSKW |
| | | EADLLRPELTFVACSAVSPAHLYMQSCENNIPPHDCVLLTHTISRPRPDLSDLPIPDPDMTLFSDGSYTTGRGG |
| | | AAVVMHRPVTDDFIIIHQQPGGASAQTAELLALAAACHLATDKTVNIYTDSRYAYGVVHDFGHLWMHRGFVTSA |
| | | GTPIKNHKEIEYLLKQIMKPKQVSVIKIEAHTKGVSMEVRGNAAADEAAKNAVFLVQR |
| WMSV_ | 8,131 | VLNLEEEYRLHEKPVPSSIDPSWLQLFPTVWAERAGMGLANQVPPVVVELRSGASPVAVRQYPMSKEAREGIRP |
| P03359 | | HIQRFLDLGVLVPCQSPWNTPLLPVKKPGTNDYRPVQDLREINKRVQDIHPTVPNPYNLLSSLPPSHTWYSVLD |
| | | LKDAFFCLKLHPNSQPLFAFEWRDPEKGNTGQLTWTRLPQGFKNSPTLFDEALHRDLAPFRALNPQVVLLQYVD |
| | | DLLVAAPTYRDCKEGTQKLLQELSKLGYRVSAKKAQLCQKEVTYLGYLLKEGKRWLTPARKATVMKIPPPTTPR |
| | | QVREFLGTAGFCRLWIPGFASLAAPLYPLTKESIPFIWTEEHQKAFDRIKEALLSAPALALPDLTKPFTLYVDE |
| | | RAGVARGVLTQTLGPWRRPVAYLSKKLDPVASGWPTCLKAVAAVALLLKDADKLTLGQNVTVIASHSLESIVRQ |
| | | PPDRWMTNARMTHYQSLLLNERVSFAPPAVLNPATLLPVESEATPVHRCSEILAEETGTRRDLKDQPLPGVPAW |
| | | YTDGSSFIAEGKRRAGAAIVDGKRTVWASSLPEGTSAQKAELVALTQALRLAEGKDINIYTDSRYAFATAHIHG |
| | | AIYKQRGLLTSAGKDIKNKEEILALLEAIHLPKRVAIIHCPGHQKGNDPVATGNRRADEAAKQAALSTRVLAET |
| | | TKP |
| WMSV_ | 8,132 | VLNLEEEYRLHEKPVPSSIDPSWLQLFPTVWAERAGMGLANQVPPVVVELRSGASPVAVRQYPMSKEAREGIRP |
| P03359_ | | HIQRFLDLGVLVPCQSPWNTPLLPVKKPGTNDYRPVQDLREINKRVQDIHPTVPNPYNLLSSLPPSHTWYSVLD |
| 3mut | | LKDAFFCLKLHPNSQPLFAFEWRDPEKGNTGQLTWTRLPQGFKNSPTLFNEALHRDLAPFRALNPQVVLLQYVD |
| | | DLLVAAPTYRDCKEGTQKLLQELSKLGYRVSAKKAQLCQKEVTYLGYLLKEGKRWLTPARKATVMKIPPPTTPR |
| | | QVREFLGTAGFCRLWIPGFASLAAPLYPLTKPSIPFIWTEEHQKAFDRIKEALLSAPALALPDLTKPFTLYVDE |
| | | RAGVARGVLTQTLGPWRRPVAYLSKKLDPVASGWPTCLKAVAAVALLLKDADKLTLGQNVTVIASHSLESIVRQ |
| | | PPDRWMTNARMTHYQSLLLNERVSFAPPAVLNPATLLPVESEATPVHRCSEILAEETGTRRDLKDQPLPGVPAW |
| | | YTDGSSFIAEGKRRAGAAIVDGKRTVWASSLPEGTSAQKAELVALTQALRLAEGKDINIYTDSRYAFATAHIHG |
| | | AIYKQRGWLTSAGKDIKNKEEILALLEAIHLPKRVAIIHCPGHQKGNDPVATGNRRADEAAKQAALSTRVLAET |
| | | TKP |
| WMSV_ | 8,133 | VLNLEEEYRLHEKPVPSSIDPSWLQLFPTVWAERAGMGLANQVPPVVVELRSGASPVAVRQYPMSKEAREGIRP |
| P03359_ | | HIQRFLDLGVLVPCQSPWNTPLLPVKKPGTNDYRPVQDLREINKRVQDIHPTVPNPYNLLSSLPPSHTWYSVLD |
| 3mutA | | LKDAFFCLKLHPNSQPLFAFEWRDPEKGNTGQLTWTRLPQGFKNSPTLFNEALHRDLAPFRALNPQVVLLQYVD |
| | | DLLVAAPTYRDCKEGTQKLLQELSKLGYRVSAKKAQLCQKEVTYLGYLLKEGKRWLTPARKATVMKIPPPTTPR |
| | | QVREFLGKAGFCRLFIPGFASLAAPLYPLTKPSIPFIWTEEHQKAFDRIKEALLSAPALALPDLTKPFTLYVDE |
| | | RAGVARGVLTQTLGPWRRPVAYLSKKLDPVASGWPTCLKAVAAVALLLKDADKLTLGQNVTVIASHSLESIVRQ |
| | | PPDRWMTNARMTHYQSLLLNERVSFAPPAVLNPATLLPVESEATPVHRCSEILAEETGTRRDLKDQPLPGVPAW |
| | | YTDGSSFIAEGKRRAGAAIVDGKRTVWASSLPEGTSAQKAELVALTQALRLAEGKDINIYTDSRYAFATAHIHG |
| | | AIYKQRGWLTSAGKDIKNKEEILALLEAIHLPKRVAIIHCPGHQKGNDPVATGNRRADEAAKQAALSTRVLAET |
| | | TKP |
| XMRV6_ | 8,134 | TLNIEDEYRLHETSKEPDVPLGSTWLSDFPQAWAETGGMGLAVRQAPLIIPLKATSTPVSIKQYPMSQEARLGI |
| A1Z651 | | KPHIQRLLDQGILVPCQSPWNTPLLPVKKPGTNDYRPVQDLREVNKRVEDIHPTVPNPYNLLSGLPPSHQWYTV |
| | | LDLKDAFFCLRLHPTSQPLFAFEWRDPEMGISGQLTWTRLPQGFKNSPTLFDEALHRDLADFRIQHPDLILLQY |
| | | VDDLLLAATSEQDCQRGTRALLQTLGNLGYRASAKKAQICQKQVKYLGYLLKEGQRWLTEARKETVMGQPTPKT |
| | | PRQLREFLGTAGFCRLWIPGFAEMAAPLYPLTKTGTLFNWGPDQQKAYQEIKQALLTAPALGLPDLTKPFELFV |
| | | DEKQGYAKGVLTQKLGPWRRPVAYLSKKLDPVAAGWPPCLRMVAAIAVLTKDAGKLTMGQPLVILAPHAVEALV |
| | | KQPPDRWLSNARMTHYQAMLLDTDRVQFGPVVALNPATLLPLPEKEAPHDCLEILAETHGTRPDLTDQPIPDAD |
| | | YTWYTDGSSFLQEGQRRAGAAVTTETEVIWARALPAGTSAQRAELIALTQALKMAEGKKLNVYTDSRYAFATAH |
| | | VHGEIYRRRGLLTSEGREIKNKNEILALLKALFLPKRLSIIHCPGHQKGNSAEARGNRMADQAAREAAMKAVLE |
| | | TSTLL |
| XMRV6_ | 8,135 | TLNIEDEYRLHETSKEPDVPLGSTWLSDFPQAWAETGGMGLAVRQAPLIIPLKATSTPVSIKQYPMSQEARLGI |
| A1Z651_ | | KPHIQRLLDQGILVPCQSPWNTPLLPVKKPGTNDYRPVQDLREVNKRVEDIHPTVPNPYNLLSGLPPSHQWYTV |
| 3mut | | LDLKDAFFCLRLHPTSQPLFAFEWRDPEMGISGQLTWTRLPQGFKNSPTLFNEALHRDLADFRIQHPDLILLQY |
| | | VDDLLLAATSEQDCQRGTRALLQTLGNLGYRASAKKAQICQKQVKYLGYLLKEGQRWLTEARKETVMGQPTPKT |
| | | PRQLREFLGTAGFCRLWIPGFAEMAAPLYPLTKPGTLFNWGPDQQKAYQEIKQALLTAPALGLPDLTKPFELFV |
| | | DEKQGYAKGVLTQKLGPWRRPVAYLSKKLDPVAAGWPPCLRMVAAIAVLTKDAGKLTMGQPLVILAPHAVEALV |
| | | KQPPDRWLSNARMTHYQAMLLDTDRVQFGPVVALNPATLLPLPEKEAPHDCLEILAETHGTRPDLTDQPIPDAD |
| | | YTWYTDGSSFLQEGQRRAGAAVTTETEVIWARALPAGTSAQRAELIALTQALKMAEGKKLNVYTDSRYAFATAH |
| | | VHGEIYRRRGWLTSEGREIKNKNEILALLKALFLPKRLSIIHCPGHQKGNSAEARGNRMADQAAREAAMKAVLE |
| | | TSTLL |
| XMRV6_ | 8,136 | TLNIEDEYRLHETSKEPDVPLGSTWLSDFPQAWAETGGMGLAVRQAPLIIPLKATSTPVSIKQYPMSQEARLGI |
| A1Z651_ | | KPHIQRLLDQGILVPCQSPWNTPLLPVKKPGTNDYRPVQDLREVNKRVEDIHPTVPNPYNLLSGLPPSHQWYTV |
| 3mutA | | LDLKDAFFCLRLHPTSQPLFAFEWRDPEMGISGQLTWTRLPQGFKNSPTLFNEALHRDLADFRIQHPDLILLQY |
| | | VDDLLLAATSEQDCQRGTRALLQTLGNLGYRASAKKAQICQKQVKYLGYLLKEGQRWLTEARKETVMGQPTPKT |
| | | PRQLREFLGKAGFCRLFIPGFAEMAAPLYPLTKPGTLFNWGPDQQKAYQEIKQALLTAPALGLPDLTKPFELFV |
| | | DEKQGYAKGVLTQKLGPWRRPVAYLSKKLDPVAAGWPPCLRMVAAIAVLTKDAGKLTMGQPLVILAPHAVEALV |
| | | KQPPDRWLSNARMTHYQAMLLDTDRVQFGPVVALNPATLLPLPEKEAPHDCLEILAETHGTRPDLTDQPIPDAD |
| | | YTWYTDGSSFLQEGQRRAGAAVTTETEVIWARALPAGTSAQRAELIALTQALKMAEGKKLNVYTDSRYAFATAH |
| | | VHGEIYRRRGWLTSEGREIKNKNEILALLKALFLPKRLSIIHCPGHQKGNSAEARGNRMADQAAREAAMKAVLE |
| | | TSTLL |

In some embodiments, reverse transcriptase domains are modified, for example by site-specific mutation. In some embodiments, reverse transcriptase domains are engineered to have improved properties, e.g. SuperScript IV (SSIV) reverse transcriptase derived from the MMLV RT. In some embodiments, the reverse transcriptase domain may be engineered to have lower error rates, e.g., as described in WO2001068895, incorporated herein by reference. In some embodiments, the reverse transcriptase domain may be engineered to be more thermostable. In some embodiments, the reverse transcriptase domain may be engineered to be more processive. In some embodiments, the reverse transcriptase domain may be engineered to have tolerance to inhibitors. In some embodiments, the reverse transcriptase domain may be engineered to be faster. In some embodiments, the reverse transcriptase domain may be engineered to better tolerate modified nucleotides in the RNA template. In some embodiments, the reverse transcriptase domain may be engineered to insert modified DNA nucleotides. In some embodiments, the reverse transcriptase domain is engineered to bind a template RNA. In some embodiments, one or more mutations are chosen from D200N, L603W, T330P, D524G, E562Q, D583N, P51L, S67R, E67K, T197A, H204R, E302K, F309N, W313F, L435G, N454K, H594Q, L671P, E69K, or D653N in the RT domain of murine leukemia virus reverse transcriptase or a corresponding mutation at a corresponding position of another RT domain.

In some embodiments, a heterologous gene modifying polypeptide comprises the RT domain from a retroviral reverse transcriptase, e.g., a wild-type M-MLV RT, e.g., comprising the following sequence:

M-MLV (WT):
(SEQ ID NO: 5002)
TLNIEDEYRLHETSKEPDVSLGSTWLSDFPQAWAETGGMGLAVRQAPLII
PLKATSTPVSIKQYPMSQEARLGIKPHIQRLLDQGILVPCQSPWNTPLLP
VKKPGTNDYRPVQDLREVNKRVEDIHPTVPNPYNLLSGLPPSHQWYTVLD
LKDAFFCLRLHPTSQPLFAFEWRDPEMGISGQLTWTRLPQGFKNSPTLFD
EALHRDLADFRIQHPDLILLQYVDDLLLAATSELDCQQGTRALLQTLGNL
GYRASAKKAQICQKQVKYLGYLLKEGQRWLTEARKETVMGQPTPKTPRQL
REFLGTAGFCRLWIPGFAEMAAPLYPLTKTGTLFNWGPDQQKAYQEIKQA
LLTAPALGLPDLTKPFELFVDEKQGYAKGVLTQKLGPWRRPVAYLSKKLD
PVAAGWPPCLRMVAAIAVLTKDAGKLTMGQPLVILAPHAVEALVKQPPDR
WLSNARMTHYQALLLDTDRVQFGPVVALNPATLLPLPEEGLQHNCLDILA
EAHGTRPDLTDQPLPDADHTWYTDGSSLLQEGQRKAGAAVTTETEVIWAK
ALPAGTSAQRAELIALTQALKMAEGKKLNVYTDSRYAFATAHIHGEIYRR
RGLLTSEGKEIKNKDEILALLKALFLPKRLSIIHCPGHQKGHSAEARGNR
MADQAARKAAITETPDTSTLLI

In some embodiments, a heterologous gene modifying polypeptide comprises the RT domain from a retroviral reverse transcriptase, e.g., an M-MLV RT, e.g., comprising the following sequence:

(SEQ ID NO: 5003)
TLNIEDEHRLHETSKEPDVSLGSTWLSDFPQAWAETGGMGLAVRQAPLII
PLKATSTPVSIKQYPMSQEARLGIKPHIQRLLDQGILVPCQSPWNTPLLP
VKKPGTNDYRPVQDLREVNKRVEDIHPTVPNPYNLLSGLPPSHQWYTVLD
LKDAFFCLRLHPTSQPLFAFEWRDPEMGISGQLTWTRLPQGFKNSPTLFD
EALHRDLADFRIQHPDLILLQYVDDLLLAATSELDCQQGTRALLQTLGNL
GYRASAKKAQICQKQVKYLGYLLKEGQRWLTEARKETVMGQPTPKTPRQL
REFLGTAGFCRLWIPGFAEMAAPLYPLTKTGTLFNWGPDQQKAYQEIKQA
LLTAPALGLPDLTKPFELFVDEKQGYAKGVLTQKLGPWRRPVAYLSKKLD
PVAAGWPPCLRMVAAIAVLTKDAGKLTMGQPLVILAPHAVEALVKQPPDR
WLSNARMTHYQALLLDTDRVQFGPVVALNPATLLPLPEEGLQHNCLDILA
EAHGTRPDLTDQPLPDADHTWYTDGSSLLQEGQRKAGAAVTTETEVIWAK
ALPAGTSAQRAELIALTQALKMAEGKKLNVYTDSRYAFATAHIHGEIYRR
RGLLTSEGKEIKNKDEILALLKALFLPKRLSIIHCPGHQKGHSAEARGNR
MADQAARKAAITETPDTSTLL

In some embodiments, a heterologous gene modifying polypeptide comprises the RT domain from a retroviral reverse transcriptase comprising the sequence of amino acids 659-1329 of NP_057933. In embodiments, the heterologous gene modifying polypeptide further comprises one additional amino acid at the N-terminus of the sequence of amino acids 659-1329 of NP_057933, e.g., as shown below:

(SEQ ID NO: 5004)
TLNIEDEHRLHETSKEPDVSLGSTWLSDFPQAWAETGGMGLAVRQAPLII
PLKATSTPVSIKQYPMSQEARLGIKPHIQRLLDQGILVPCQSPWNTPLLP
VKKPGTNDYRPVQDLREVNKRVEDIHPTVPNPYNLLSGLPPSHQWYTVLD
LKDAFFCLRLHPTSQPLFAFEWRDPEMGISGQLTWTRLPQGFKNSPTLFD
EALHRDLADFRIQHPDLILLQYVDDLLLAATSELDCQQGTRALLQTLGNL
GYRASAKKAQICQKQVKYLGYLLKEGQRWLTEARKETVMGQPTPKTPRQL
REFLGTAGFCRLWIPGFAEMAAPLYPLTKTGTLFNWGPDQQKAYQEIKQA
LLTAPALGLPDLTKPFELFVDEKQGYAKGVLTQKLGPWRRPVAYLSKKLD
PVAAGWPPCLRMVAAIAVLTKDAGKLTMGQPLVILAPHAVEALVKQPPDR
WLSNARMTHYQALLLDTDRVQFGPVVALNPATLLPLPEEGLQHNCLDILA
EAHGTRPDLTDQPLPDADHTWYTDGSSLLQEGORKAGAAVTTETEVIWAK
ALPAGTSAQRAELIALTQALKMAEGKKLNVYTDSRYAFATAHIHGEIYRR
RGLLTSEGKEIKNKDEILALLKALFLPKRLSIIHCPGHQKGHSAEARGNR
MADQAARKAA
Core RT (bold), annotated per above
RNAseH (underlined), annotated per above

In embodiments, the heterologous gene modifying polypeptide further comprises one additional amino acid at the C-terminus of the sequence of amino acids 659-1329 of NP_057933. In embodiments, the heterologous gene modifying polypeptide comprises an RNaseH1 domain (e.g., amino acids 1178-1318 of NP_057933).

In some embodiments, a retroviral reverse transcriptase domain, e.g., M-MLV RT, may comprise one or more mutations from a wild-type sequence that may improve features of the RT, e.g., thermostability, processivity, and/or template binding. In some embodiments, an M-MLV RT domain comprises, relative to the M-MLV (WT) sequence above, one or more mutations, e.g., selected from D200N, L603W, T330P, T306K, W313F, D524G, E562Q, D583N, P51L, S67R, E67K, T197A, H204R, E302K, F309N, L435G, N454K, H594Q, D653N, R110S, K103L, e.g., a combination of mutations, such as D200N, L603W, and T330P, optionally further including T306K and W313F. In some embodiments, an M-MLV RT used herein comprises the mutations D200N, L603W, T330P, T306K and W313F. In embodiments, the mutant M-MLV RT comprises the following amino acid sequence:

M-MLV (PE2):
(SEQ ID NO: 5005)
TLNIEDEYRLHETSKEPDVSLGSTWLSDFPQAWAETGGMGLAVRQAPLII
PLKATSTPVSIKQYPMSQEARLGIKPHIQRLLDQGILVPCQSPWNTPLLP
VKKPGTNDYRPVQDLREVNKRVEDIHPTVPNPYNLLSGLPPSHQWYTVLD
LKDAFFCLRLHPTSQPLFAFEWRDPEMGISGQLTWTRLPQGFKNSPTLFN
EALHRDLADFRIQHPDLILLQYVDDLLLAATSELDCQQGTRALLQTLGNL
GYRASAKKAQICQKQVKYLGYLLKEGQRWLTEARKETVMGQPTPKTPRQL
REFLGKAGFCRLFIPGFAEMAAPLYPLTKPGTLFNWGPDQQKAYQEIKQA
LLTAPALGLPDLTKPFELFVDEKQGYAKGVLTQKLGPWRRPVAYLSKKLD
PVAAGWPPCLRMVAAIAVLTKDAGKLTMGQPLVILAPHAVEALVKQPPDR
WLSNARMTHYQALLLDTDRVQFGPVVALNPATLLPLPEEGLQHNCLDILA
EAHGTRPDLTDQPLPDADHTWYTDGSSLLQEGQRKAGAAVTTETEVIWAK
ALPAGTSAQRAELIALTQALKMAEGKKLNVYTDSRYAFATAHIHGEIYRR
RGWLTSEGKEIKNKDEILALLKALFLPKRLSIIHCPGHQKGHSAEARGNR
MADQAARKAAITETPDTSTLLI

In some embodiments, a writing domain (e.g., RT domain) comprises an RNA-binding domain, e.g., that specifically binds to an RNA sequence. In some embodiments, a template RNA comprises an RNA sequence that is specifically bound by the RNA-binding domain of the writing domain.

In some embodiments, the reverse transcription domain only recognizes and reverse transcribes a specific template, e.g., a template RNA of the system. In some embodiments, the template comprises a sequence or structure that enables recognition and reverse transcription by a reverse transcription domain. In some embodiments, the template comprises a sequence or structure that enables association with an RNA-binding domain of a polypeptide component of a genome engineering system described herein. In some embodiments, the genome engineering system reverse preferably transcribes a template comprising an association sequence over a template lacking an association sequence.

The writing domain may also comprise DNA-dependent DNA polymerase activity, e.g., comprise enzymatic activity capable of writing DNA into the genome from a template DNA sequence. In some embodiments, DNA-dependent DNA polymerization is employed to complete second-strand synthesis of a target site edit. In some embodiments, the DNA-dependent DNA polymerase activity is provided by a DNA polymerase domain in the polypeptide. In some embodiments, the DNA-dependent DNA polymerase activity is provided by a reverse transcriptase domain that is also capable of DNA-dependent DNA polymerization, e.g., second-strand synthesis. In some embodiments, the DNA-dependent DNA polymerase activity is provided by a second polypeptide of the system. In some embodiments, the DNA-dependent DNA polymerase activity is provided by an endogenous host cell polymerase that is optionally recruited to the target site by a component of the genome engineering system.

In some embodiments, the reverse transcriptase domain has a lower probability of premature termination rate (P_{off}) in vitro relative to a reference reverse transcriptase domain. In some embodiments, the reference reverse transcriptase domain is a viral reverse transcriptase domain, e.g., the RT domain from M-MLV.

In some embodiments, the reverse transcriptase domain has a lower probability of premature termination rate (P_{off}) in vitro of less than about 5×10⁻³/nt, 5×10⁻⁴/nt, or 5×10⁻⁶/nt, e.g., as measured on a 1094 nt RNA. In embodiments, the in vitro premature termination rate is determined as described in Bibillo and Eickbush (2002) J Biol Chem 277 (38): 34836-34845 (incorporated by reference herein its entirety).

In some embodiments, the reverse transcriptase domain is able to complete at least about 30% or 50% of integrations in cells. The percent of complete integrations can be measured by dividing the number of substantially full-length integration events (e.g., genomic sites that comprise at least 98% of the expected integrated sequence) by the number of total (including substantially full-length and partial) integration events in a population of cells. In embodiments, the integrations in cells is determined (e.g., across the integration site) using long-read amplicon sequencing, e.g., as described in Karst et al. (2020) bioRxiv doi.org/10.1101/645903 (incorporated by reference herein in its entirety).

In embodiments, quantifying integrations in cells comprises counting the fraction of integrations that contain at least about 75%, 80%, 85%, 90%, 95%, 96%, 97%, 98%, 99%, or 100% of the DNA sequence corresponding to the template RNA (e.g., a template RNA having a length of at least 0.05, 0.1, 0.5, 0.6, 0.7, 0.8, 0.9, 1, 1.5, 2, 3, 4, or 5 kb, e.g., a length between 0.5-0.6, 0.6-0.7, 0.7-0.8, 0.8-0.9, 1.0-1.2, 1.2-1.4, 1.4-1.6, 1.6-1.8, 1.8-2.0, 2-3, 3-4, or 4-5 kb).

In some embodiments, the reverse transcriptase domain is capable of polymerizing dNTPs in vitro. In embodiments, the reverse transcriptase domain is capable of polymerizing dNTPs in vitro at a rate between 0.1-50 nt/see (e.g., between 0.1-1, 1-10, or 10-50 nt/sec). In embodiments, polymerization of dNTPs by the reverse transcriptase domain is measured by a single-molecule assay, e.g., as described in Schwartz and Quake (2009) PNAS 106 (48): 20294-20299 (incorporated by reference in its entirety).

In some embodiments, the reverse transcriptase domain has an in vitro error rate (e.g., misincorporation of nucleotides) of between 1×10⁻³-1×10⁻⁴ or 1×10⁻⁴-1×10⁻⁵ substitutions/nt, e.g., as described in Yasukawa et al. (2017) Biochem Biophys Res Commun 492 (2): 147-153 (incorporated herein by reference in its entirety). In some embodiments, the reverse transcriptase domain has an error rate (e.g., misincorporation of nucleotides) in cells (e.g., HEK293T cells) of between 1×10⁻³-1×10⁻⁴ or 1×10⁻⁴-1×10⁻⁵ substitutions/nt, e.g., by long-read amplicon sequencing, e.g., as described in Karst et al. (2020) bioRxiv doi.org/10.1101/645903 (incorporated by reference herein in its entirety).

In some embodiments, the reverse transcriptase domain is capable of performing reverse transcription of a target RNA in vitro. In some embodiments, the reverse transcriptase requires a primer of at least 3 nucleotides to initiate reverse transcription of a template. In some embodiments, reverse transcription of the target RNA is determined by detection of cDNA from the target RNA (e.g., when provided with a ssDNA primer, e.g., which anneals to the target with at least 3, 4, 5, 6, 7, 8, 9, or 10 nt at the 3′ end), e.g., as described in Bibillo and Eickbush (2002) J Biol Chem 277 (38): 34836-34845 (incorporated herein by reference in its entirety).

In some embodiments, the reverse transcriptase domain performs reverse transcription at least 5 or 10 times more efficiently (e.g., by cDNA production), e.g., when converting its RNA template to cDNA, for example, as compared to an RNA template lacking the protein binding motif (e.g., a 3′ UTR). In embodiments, efficiency of reverse transcription is measured as described in Yasukawa et al. (2017) Biochem Biophys Res Commun 492 (2): 147-153 (incorporated by reference herein in its entirety).

In some embodiments, the reverse transcriptase domain specifically binds a specific RNA template with higher frequency (e.g., about 5 or 10-fold higher frequency) than any endogenous cellular RNA, e.g., when expressed in cells (e.g., HEK293T cells). In embodiments, frequency of specific binding between the reverse transcriptase domain and the template RNA are measured by CLIP-seq, e.g., as described in Lin and Miles (2019) Nucleic Acids Res 47 (11): 5490-5501 (incorporated herein by reference in its entirety).

In some embodiments, an RT domain (e.g., as listed in Table 6) comprises one or more mutations as listed in Table 2A1 below. In some embodiment, an RT domain as listed in Table 6 comprises one, two, three, four, five, or six of the mutations listed in the corresponding row of Table 2A1 below.

TABLE 2A1
Exemplary RT domain mutations (relative to corresponding wild-type
sequences as listed in the corresponding row of Table 6)

| RT Domain Name | Mutation(s) | | | | | |
|---|---|---|---|---|---|---|
| AVIRE_P03360 | | | | | | |
| AVIRE_P03360_3mut | D200N | G330P | L605W | | | |
| AVIRE_P03360_3mutA | D200N | G330P | L605W | T306K | W313F | |
| BAEVM_P10272 | | | | | | |
| BAEVM_P10272_3mut | D198N | E328P | L602W | | | |
| BAEVM_P10272_3mutA | D198N | E328P | L602W | T304K | W311F | |
| BLVAU_P25059 | | | | | | |
| BLVAU_P25059_2mut | E159Q | G286P | | | | |
| BLVJ_P03361 | | | | | | |
| BLVJ_P03361_2mut | E159Q | L524W | | | | |
| BLVJ_P03361_2mutB | E159Q | L524W | 197P | | | |
| FFV_093209 | D21N | | | | | |
| FFV_093209_2mut | D21N | T293N | T419P | | | |
| FFV_093209_2mutA | D21N | T293N | T419P | L393K | | |
| FFV_093209-Pro | | | | | | |
| FFV_093209-Pro_2mut | T207N | T333P | | | | |
| FFV_093209-Pro_2mutA | T207N | T333P | L307K | | | |
| FLV_P10273 | | | | | | |
| FLV_P10273_3mut | D199N | L602W | | | | |
| FLV_P10273_3mutA | D199N | L602W | T305K | W312F | | |
| FOAMV_P14350 | D24N | | | | | |
| FOAMV_P14350_2mut | D24N | T296N | S420P | | | |
| FOAMV_P14350_2mutA | D24N | T296N | S420P | L396K | | |
| FOAMV_P14350-Pro | | | | | | |
| FOAMV_P14350-Pro_2mut | T207N | S331P | | | | |
| FOAMV_P14350-Pro_2mutA | T207N | S331P | L307K | | | |
| GALV_P21414 | | | | | | |
| GALV_P21414_3mut | D198N | E328P | L600W | | | |
| GALV_P21414_3mutA | D198N | E328P | L600W | T304K | W311F | |
| HTL1A_P03362 | | | | | | |
| HTL1A_P03362_2mut | E152Q | R279P | | | | |
| HTL1A_P03362_2mutB | E152Q | R279P | L90P | | | |
| HTL1C_P14078 | | | | | | |
| HTL1C_P14078_2mut | E152Q | R279P | | | | |
| HTL1L_P0C211 | | | | | | |
| HTL1L_P0C211_2mut | E149Q | L527W | | | | |
| HTL1L_P0C211_2mutB | E149Q | L527W | L87P | | | |
| HTL32_Q0R5R2 | | | | | | |
| HTL32_Q0R5R2_2mut | E149Q | L526W | | | | |
| HTL32_Q0R5R2_2mutB | E149Q | L526W | L87P | | | |
| HTL3P_Q4U0X6 | | | | | | |
| HTL3P_Q4U0X6_2mut | E149Q | L526W | | | | |
| HTL3P_Q4U0X6_2mutB | E149Q | L526W | L87P | | | |
| HTLV2_P03363_2mut | E147Q | G274P | | | | |
| JSRV_P31623 | | | | | | |
| JSRV_P31623_2mutB | A100P | | | | | |
| KORV_Q9TTC1 | D32N | | | | | |
| KORV_Q9TTC1_3mut | D32N | D322N | E452P | L724W | | |
| KORV_Q9TTC1_3mutA | D32N | D322N | E452P | L724W | T428K | W435F |
| KORV_Q9TTC1-Pro | | | | | | |
| KORV_Q9TTC1-Pro_3mut | D231N | E361P | L633W | | | |
| KORV_Q9TTC1-Pro_3mutA | D231N | E361P | L633W | T337K | W344F | |
| MLVAV_P03356 | | | | | | |
| MLVAV_P03356_3mut | D200N | T330P | L603W | | | |
| MLVAV_P03356_3mutA | D200N | T330P | L603W | T306K | W313F | |
| MLVBM_Q7SVK7 | | | | | | |
| MLVBM_Q7SVK7 | | | | | | |
| MLVBM_Q7SVK7_3mut | D200N | T330P | L603W | | | |
| MLVBM_Q7SVK7_3mut | D200N | T330P | L603W | | | |
| MLVBM_Q7SVK7_3mutA_WS | D199N | T329P | L602W | T305K | W312F | |
| MLVBM_Q7SVK7_3mutA_WS | D199N | T329P | L602W | T305K | W312F | |
| MLVCB_P08361 | | | | | | |
| MLVCB_P08361_3mut | D200N | T330P | L603W | | | |
| MLVCB_P08361_3mutA | D200N | T330P | L603W | T306K | W313F | |
| MLVF5_P26810 | | | | | | |
| MLVF5_P26810_3mut | D200N | T330P | L603W | | | |
| MLVF5_P26810_3mutA | D200N | T330P | L603W | T306K | W313F | |
| MLVFF_P26809_3mut | D200N | T330P | L603W | | | |
| MLVFF_P26809_3mutA | D200N | T330P | L603W | T306K | W313F | |
| MLVMS_P03355 | | | | | | |
| MLVMS_P03355 | | | | | | |
| MLVMS_P03355_3mut | D200N | T330P | L603W | | | |
| MLVMS_P03355_3mut | D200N | T330P | L603W | | | |
| MLVMS_P03355_3mutA_WS | D200N | T330P | L603W | T306K | W313F | |
| MLVMS_P03355_3mutA_WS | D200N | T330P | L603W | T306K | W313F | |
| MLVMS_P03355_PLV919 | D200N | T330P | L603W | T306K | W313F | H8Y |
| MLVMS_P03355_PLV919 | D200N | T330P | L603W | T306K | W313F | H8Y |
| MLVRD_P11227 | | | | | | |
| MLVRD_P11227_3mut | D200N | T330P | L603W | | | |
| MMTVB_P03365 | D26N | | | | | |
| MMTVB_P03365 | D26N | | | | | |
| MMTVB_P03365_2mut | D26N | G401P | | | | |
| MMTVB_P03365_2mut_WS | G400P | | | | | |
| MMTVB_P03365_2mut_WS | G400P | | | | | |
| MMTVB_P03365_2mutB | D26N | G401P | V215P | | | |
| MMTVB_P03365_2mutB | D26N | G401P | V215P | | | |
| MMTVB_P03365_2mutB_WS | G400P | V212P | | | | |
| MMTVB_P03365_2mutB_WS | G400P | V212P | | | | |
| MMTVB_P03365_WS | | | | | | |
| MMTVB_P03365_WS | | | | | | |
| MMTVB_P03365-Pro | | | | | | |
| MMTVB_P03365-Pro | | | | | | |
| MMTVB_P03365-Pro_2mut | G309P | | | | | |
| MMTVB_P03365-Pro_2mut | G309P | | | | | |
| MMTVB_P03365-Pro_2mutB | G309P | V123P | | | | |
| MMTVB_P03365-Pro_2mutB | G309P | V123P | | | | |
| MPMV_P07572 | | | | | | |
| MPMV_P07572_2mutB | G289P | I103P | | | | |
| PERV_Q4VFZ2 | | | | | | |
| PERV_Q4VFZ2 | | | | | | |
| PERV_Q4VFZ2_3mut | D199N | E329P | L602W | | | |
| PERV_Q4VFZ2_3mut | D199N | E329P | L602W | | | |
| PERV_Q4VFZ2_3mutA_WS | D196N | E326P | L599W | T302K | W309F | |
| PERV_Q4VFZ2_3mutA_WS | D196N | E326P | L599W | T302K | W309F | |
| SFV1_P23074 | D24N | | | | | |
| SFV1_P23074_2mut | D24N | T296N | N420P | | | |
| SFV1_P23074_2mutA | D24N | T296N | N420P | L396K | | |
| SFV1_P23074-Pro | | | | | | |
| SFV1_P23074-Pro_2mut | T207N | N331P | | | | |
| SFV1_P23074-Pro_2mutA | T207N | N331P | L307K | | | |
| SFV3L_P27401 | D24N | | | | | |
| SFV3L_P27401_2mut | D24N | T296N | N422P | | | |
| SFV3L_P27401_2mutA | D24N | T296N | N422P | L396K | | |
| SFV3L_P27401-Pro | | | | | | |
| SFV3L_P27401-Pro_2mut | T307N | N333P | | | | |
| SFV3L_P27401-Pro_2mutA | T307N | N333P | L307K | | | |
| SFVCP_Q87040 | D24N | | | | | |
| SFVCP_Q87040_2mut | D24N | T296N | K422P | | | |
| SFVCP_Q87040_2mutA | D24N | T296N | K422P | L396K | | |
| SFVCP_Q87040-Pro | | | | | | |
| SFVCP_Q87040-Pro_2mut | T207N | K333P | | | | |
| SFVCP_Q87040-Pro_2mutA | T207N | K333P | L307K | | | |
| SMRVH_P03364 | | | | | | |
| SMRVH_P03364_2mut | G288P | | | | | |
| SMRVH_P03364_2mutB | G288P | I102P | | | | |
| SRV2_P51517 | | | | | | |
| SRV2_P51517_2mutB | I103P | | | | | |
| WDSV_O92815 | | | | | | |
| WDSV_O92815_2mut | S183N | K312P | | | | |
| WDSV_O92815_2mutA | S183N | K312P | L288K | W295F | | |
| WMSV_P03359 | | | | | | |
| WMSV_P03359_3mut | D198N | E328P | L600W | | | |
| WMSV_P03359_3mutA | D198N | E328P | L600W | T304K | W311F | |
| XMRV6_A1Z651 | | | | | | |
| XMRV6_A1Z651_3mut | D200N | T330P | L603W | | | |
| XMRV6_A1Z651_3mutA | D200N | T330P | L603W | T306K | W313F | |

### Template Nucleic Acid Binding Domain

The heterologous gene modifying polypeptide typically contains regions capable of associating with the template nucleic acid (e.g., template RNA). In some embodiments, the template nucleic acid binding domain is an RNA binding domain. In some embodiments, the RNA binding domain is a modular domain that can associate with RNA molecules containing specific signatures, e.g., structural motifs. In other embodiments, the template nucleic acid binding domain (e.g., RNA binding domain) is contained within the reverse transcription domain, e.g., the reverse transcriptase-derived component has a known signature for RNA preference.

In other embodiments, the template nucleic acid binding domain (e.g., RNA binding domain) is contained within the target DNA binding domain. For example, in some embodiments, the DNA binding domain is a CRISPR-associated protein that recognizes the structure of a template nucleic acid (e.g., template RNA) comprising a gRNA. In some embodiments, a heterologous gene modifying polypeptide comprises a DNA-binding domain comprising a CRISPR-associated protein that associates with a gRNA scaffold that allows the DNA-binding domain to bind a target genomic DNA sequence. In some embodiments, the gRNA scaffold and gRNA spacer is comprised within the template nucleic acid (e.g., template RNA), thus the DNA-binding domain is also the template nucleic acid binding domain. In some embodiments, the polypeptide possesses RNA binding function in multiple domains, e.g., can bind a gRNA structure in a CRISPR-associated DNA binding domain and an additional sequence or structure in a reverse transcriptase domain.

In some embodiments, the RNA binding domain is capable of binding to a template RNA with greater affinity than a reference RNA binding domain. In some embodiments, the reference RNA binding domain is an RNA binding domain from Cas9 of S. pyogenes. In some embodiments, the RNA binding domain is capable of binding to a template RNA with an affinity between 100 μM-10 nM (e.g., between 100 μM-1 nM or 1 nM-10 nM). In some embodiments, the affinity of a RNA binding domain for its template RNA is measured in vitro, e.g., by thermophoresis, e.g., as described in Asmari et al. Methods 146:107-119 (2018) (incorporated by reference herein in its entirety). In some embodiments, the affinity of a RNA binding domain for its template RNA is measured in cells (e.g., by FRET or CLIP-Seq).

In some embodiments, the RNA binding domain is associated with the template RNA in vitro at a frequency at least about 5-fold or 10-fold higher than with a scrambled RNA. In some embodiments, the frequency of association between the RNA binding domain and the template RNA or scrambled RNA is measured by CLIP-seq, e.g., as described in Lin and Miles (2019) Nucleic Acids Res 47 (11): 5490-5501 (incorporated by reference herein in its entirety). In some embodiments, the RNA binding domain is associated with the template RNA in cells (e.g., in HEK293T cells) at a frequency at least about 5-fold or 10-fold higher than with a scrambled RNA. In some embodiments, the frequency of association between the RNA binding domain and the template RNA or scrambled RNA is measured by CLIP-seq, e.g., as described in Lin and Miles (2019), supra.

### Endonuclease Domains and DNA Binding Domains

In some embodiments, a heterologous gene modifying polypeptide possesses the function of DNA target site cleavage via an endonuclease domain. In some embodiments, a heterologous gene modifying polypeptide comprises a DNA binding domain, e.g., for binding to a target nucleic acid. In some embodiments, a domain (e.g., a Cas domain) of the heterologous gene modifying polypeptide comprises two or more smaller domains, e.g., a DNA binding domain and an endonuclease domain. It is understood that when a DNA binding domain (e.g., a Cas domain) is said to bind to a target nucleic acid sequence, in some embodiments, the binding is mediated by a gRNA.

In some embodiments, a domain has two functions. For example, in some embodiments, the endonuclease domain is also a DNA-binding domain. In some embodiments, the endonuclease domain is also a template nucleic acid (e.g., template RNA) binding domain. For example, in some embodiments, a polypeptide comprises a CRISPR-associated endonuclease domain that binds a template RNA comprising a gRNA, binds a target DNA sequence (e.g., with complementarity to a portion of the gRNA), and cuts the target DNA sequence. In some embodiments, an endonuclease domain or endonuclease/DNA-binding domain from a heterologous source can be used or can be modified (e.g., by insertion, deletion, or substitution of one or more residues) in a heterologous gene modifying system described herein.

In some embodiments, a nucleic acid encoding the endonuclease domain or endonuclease/DNA binding domain is altered from its natural sequence to have altered codon usage, e.g. improved for human cells. In some embodiments, the endonuclease element is a heterologous endonuclease element, such as a Cas endonuclease (e.g., Cas9), a type-II restriction endonuclease (e.g., Fok1), a meganuclease (e.g., I-SceI), or other endonuclease domain.

In certain aspects, the DNA-binding domain of a heterologous gene modifying polypeptide described herein is selected, designed, or constructed for binding to a desired host DNA target sequence. In certain embodiments, the DNA-binding domain of the polypeptide is a heterologous DNA-binding element. In some embodiments the heterologous DNA binding element is a zinc-finger element or a TAL effector element, e.g., a zinc-finger or TAL polypeptide or functional fragment thereof. In some embodiments the heterologous DNA binding element is a sequence-guided DNA binding element, such as Cas9, Cpf1, or other CRISPR-related protein that has been altered to have no endonuclease activity. In some embodiments the heterologous DNA binding element retains endonuclease activity. In some embodiments, the heterologous DNA binding element retains partial endonuclease activity to cleave ssDNA, e.g., possesses nickase activity. In specific embodiments, the heterologous DNA-binding domain can be any one or more of Cas9, TAL domain, ZF domain, Myb domain, combinations thereof, or multiples thereof.

In some embodiments, DNA-binding domains are modified, for example by site-specific mutation, increasing or decreasing DNA-binding elements (for example, number and/or specificity of zinc fingers), etc., to alter DNA-binding specificity and affinity. In some embodiments a nucleic acid sequence encoding the DNA binding domain is altered from its natural sequence to have altered codon usage, e.g. improved for human cells. In embodiments, the DNA binding domain comprises one or more modifications relative to a wild-type DNA binding domain, e.g., a modification via directed evolution, e.g., phage-assisted continuous evolution (PACE).

In some embodiments, the DNA binding domain comprises a meganuclease domain (e.g., as described herein, e.g., in the endonuclease domain section), or a functional fragment thereof. In some embodiments, the meganuclease domain possesses endonuclease activity, e.g., double-strand cleavage and/or nickase activity. In other embodiments, the meganuclease domain has reduced activity, e.g., lacks endonuclease activity, e.g., the meganuclease is catalytically inactive. In some embodiments, a catalytically inactive meganuclease is used as a DNA binding domain, e.g., as described in Fonfara et al. Nucleic Acids Res 40 (2): 847-860 (2012), incorporated herein by reference in its entirety.

In some embodiments, a heterologous gene modifying polypeptide comprises a modification to a DNA-binding domain, e.g., relative to the wild-type polypeptide. In some embodiments, the DNA-binding domain comprises an addition, deletion, replacement, or modification to the amino acid sequence of the original DNA-binding domain. In some embodiments, the DNA-binding domain is modified to include a heterologous functional domain that binds specifically to a target nucleic acid (e.g., DNA) sequence of interest. In some embodiments, the functional domain replaces at least a portion (e.g., the entirety of) the prior DNA-binding domain of the polypeptide. In some embodiments, the functional domain comprises a zinc finger (e.g., a zinc finger that specifically binds to the target nucleic acid (e.g., DNA) sequence of interest. In some embodiments, the functional domain comprises a Cas domain (e.g., a Cas domain that specifically binds to the target nucleic acid (e.g., DNA) sequence of interest. In some embodiments, the Cas domain comprises a Cas9 or a mutant or variant thereof (e.g., as described herein). In embodiments, the Cas domain is associated with a guide RNA (gRNA), e.g., as described herein. In embodiments, the Cas domain is directed to a target nucleic acid (e.g., DNA) sequence of interest by the gRNA. In embodiments, the Cas domain is encoded in the same nucleic acid (e.g., RNA) molecule as the gRNA. In embodiments, the Cas domain is encoded in a different nucleic acid (e.g., RNA) molecule from the gRNA.

In some embodiments, the DNA binding domain is capable of binding to a target sequence (e.g., a dsDNA target sequence) with greater affinity than a reference DNA binding domain. In some embodiments, the reference DNA binding domain is a DNA binding domain from Cas9 of S. pyogenes. In some embodiments, the DNA binding domain is capable of binding to a target sequence (e.g., a dsDNA target sequence) with an affinity between 100 μM-10 nM (e.g., between 100 μM-1 nM or 1 nM-10 nM).

In some embodiments, the affinity of a DNA binding domain for its target sequence (e.g., dsDNA target sequence) is measured in vitro, e.g., by thermophoresis, e.g., as described in Asmari et al. Methods 146:107-119 (2018) (incorporated by reference herein in its entirety).

In embodiments, the DNA binding domain is capable of binding to its target sequence (e.g., dsDNA target sequence), e.g, with an affinity between 100 μM-10 nM (e.g., between 100 pM-1 nM or 1 nM-10 nM) in the presence of a molar excess of scrambled sequence competitor dsDNA, e.g., of about 100-fold molar excess.

In some embodiments, the DNA binding domain is found associated with its target sequence (e.g., dsDNA target sequence) more frequently than any other sequence in the genome of a target cell, e.g., human target cell, e.g., as measured by ChIP-seq (e.g., in HEK293T cells), e.g., as described in He and Pu (2010) Curr. Protoc Mol Biol Chapter 21 (incorporated herein by reference in its entirety). In some embodiments, the DNA binding domain is found associated with its target sequence (e.g., dsDNA target sequence) at least about 5-fold or 10-fold, more frequently than any other sequence in the genome of a target cell, e.g., as measured by ChIP-seq (e.g., in HEK293T cells), e.g., as described in He and Pu (2010), supra.

In some embodiments, the endonuclease domain has nickase activity and cleaves one strand of a target DNA. In some embodiments, nickase activity reduces the formation of double-stranded breaks at the target site. In some embodiments, the endonuclease domain creates a staggered nick structure in the first and second strands of a target DNA. In some embodiments, a staggered nick structure generates free 3′ overhangs at the target site. In some embodiments, free 3′ overhangs at the target site improve editing efficiency, e.g., by enhancing access and annealing of a 3′ homology region of a template nucleic acid. In some embodiments, a staggered nick structure reduces the formation of double-stranded breaks at the target site.

In some embodiments, the endonuclease domain cleaves both strands of a target DNA, e.g., results in blunt-end cleavage of a target with no ssDNA overhangs on either side of the cut-site. The amino acid sequence of an endonuclease domain of a heterologous gene modifying system described herein may be at least about 50%, at least about 60%, at least about 70%, at least about 80%, at least about 85%, at least about 90%, at least about 95%, at least about 96%, at least about 97%, at least about 98%, at least about 99% identical to the amino acid sequence of an endonuclease domain described herein, e.g., an endonuclease domain from Table 8.

In certain embodiments, the heterologous endonuclease is Fok1 or a functional fragment thereof. In certain embodiments, the heterologous endonuclease is a Holliday junction resolvase or homolog thereof, such as the Holliday junction resolving enzyme from Sulfolobus solfataricus—Ssol Hje (Govindaraju et al., Nucleic Acids Research 44:7, 2016). In certain embodiments, the heterologous endonuclease is the endonuclease of the large fragment of a spliceosomal protein, such as Prp8 (Mahbub et al., Mobile DNA 8:16, 2017). In certain embodiments, the heterologous endonuclease is derived from a CRISPR-associated protein, e.g., Cas9. In certain embodiments, the heterologous endonuclease is engineered to have only ssDNA cleavage activity, e.g., only nickase activity, e.g., be a Cas9 nickase, e.g., SpCas9 with D10A, H840A, or N863A mutations. Table 8 provides exemplary Cas proteins and mutations associated with nickase activity. In still other embodiments, homologous endonuclease domains are modified, for example by site-specific mutation, to alter DNA endonuclease activity. In still other embodiments, endonuclease domains are modified to reduce DNA-sequence specificity, e.g., by truncation to remove domains that confer DNA-sequence specificity or mutation to inactivate regions conferring DNA-sequence specificity.

In some embodiments, the endonuclease domain has nickase activity and does not form double-stranded breaks. In some embodiments, the endonuclease domain forms single-stranded breaks at a higher frequency than double-stranded breaks, e.g., at least 90%, 95%, 96%, 97%, 98%, or 99% of the breaks are single-stranded breaks, or less than 10%, 5%, 4%, 3%, 2%, or 1% of the breaks are double-stranded breaks. In some embodiments, the endonuclease forms substantially no double-stranded breaks. In some embodiments, the endonuclease does not form detectable levels of double-stranded breaks.

In some embodiments, the endonuclease domain has nickase activity that nicks the target site DNA of the first strand; e.g., in some embodiments, the endonuclease domain cuts the genomic DNA of the target site near to the site of alteration on the strand that will be extended by the writing domain. In some embodiments, the endonuclease domain has nickase activity that nicks the target site DNA of the first strand and does not nick the target site DNA of the second strand. For example, when a polypeptide comprises a CRISPR-associated endonuclease domain having nickase activity, in some embodiments, said CRISPR-associated endonuclease domain nicks the target site DNA strand containing the PAM site (e.g., and does not nick the target site DNA strand that does not contain the PAM site). As a further example, when a polypeptide comprises a CRISPR-associated endonuclease domain having nickase activity, in some embodiments, said CRISPR-associated endonuclease domain nicks the target site DNA strand not containing the PAM site (e.g., and does not nick the target site DNA strand that contains the PAM site).

In some other embodiments, the endonuclease domain has nickase activity that nicks the target site DNA of the first strand and the second strand. Without wishing to be bound by theory, after a writing domain (e.g., RT domain) of a polypeptide described herein polymerizes (e.g., reverse transcribes) from the heterologous object sequence of a template nucleic acid (e.g., template RNA), the cellular DNA repair machinery must repair the nick on the first DNA strand. The target site DNA now contains two different sequences for the first DNA strand: one corresponding to the original genomic DNA (e.g., having a free 5′ end) and a second corresponding to that polymerized from the heterologous object sequence (e.g., having a free 3′ end). It is thought that the two different sequences equilibrate with one another, first one hybridizing the second strand, then the other, and which sequence the cellular DNA repair apparatus incorporates into its repaired target site may be a stochastic process. Without wishing to be bound by theory, it is thought that introducing an additional nick to the second-strand may bias the cellular DNA repair machinery to adopt the heterologous object sequence-based sequence more frequently than the original genomic sequence (Anzalone et al. Nature 576:149-157 (2019)). In some embodiments, the additional nick is positioned at least 10, 15, 20, 25, 30, 35, 40, 45, 50, 55, 60, 65, 70, 75, 80, 85, 90, 95, 100, 105, 110, 115, 120, 125, 130, 135, 140, 145, or 150 nucleotides 5′ or 3′ of the target site modification (e.g., the insertion, deletion, or substitution) or to the nick on the first strand.

Alternatively or additionally, without wishing to be bound by theory, it is thought that an additional nick to the second strand may promote second-strand synthesis. In some embodiments, where the heterologous gene modifying system has inserted or substituted a portion of the first strand, synthesis of a new sequence corresponding to the insertion/substitution in the second strand is necessary.

In some embodiments, the polypeptide comprises a single domain having endonuclease activity (e.g., a single endonuclease domain) and said domain nicks both the first strand and the second strand. For example, in such an embodiment the endonuclease domain may be a CRISPR-associated endonuclease domain, and the template nucleic acid (e.g., template RNA) comprises a gRNA spacer that directs nicking of the first strand and an additional gRNA spacer that directs nicking of the second strand. In some embodiments, the polypeptide comprises a plurality of domains having endonuclease activity, and a first endonuclease domain nicks the first strand and a second endonuclease domain nicks the second strand (optionally, the first endonuclease domain does not (e.g., cannot) nick the second strand and the second endonuclease domain does not (e.g., cannot) nick the first strand).

In some embodiments, the endonuclease domain is capable of nicking a first strand and a second strand. In some embodiments, the first and second strand nicks occur at the same position in the target site but on opposite strands. In some embodiments, the second strand nick occurs in a staggered location, e.g., upstream or downstream, from the first nick. In some embodiments, the endonuclease domain generates a target site deletion if the second strand nick is upstream of the first strand nick. In some embodiments, the endonuclease domain generates a target site duplication if the second strand nick is downstream of the first strand nick. In some embodiments, the endonuclease domain generates no duplication and/or deletion if the first and second strand nicks occur in the same position of the target site. In some embodiments, the endonuclease domain has altered activity depending on protein conformation or RNA-binding status, e.g., which promotes the nicking of the first or second strand (e.g., as described in Christensen et al. PNAS 2006; incorporated by reference herein in its entirety).

In some embodiments, the endonuclease domain comprises a meganuclease, or a functional fragment thereof. In some embodiments, the endonuclease domain comprises a homing endonuclease, or a functional fragment thereof. In some embodiments, the endonuclease domain comprises a meganuclease from the LAGLIDADG (SEQ ID NO: 37638), GIY-YIG, HNH, His-Cys Box, or PD-(D/E) XK families, or a functional fragment or variant thereof, e.g., which possess conserved amino acid motifs, e.g., as indicated in the family names. In some embodiments, the endonuclease domain comprises a meganuclease, or fragment thereof, chosen from, e.g., I-SmaMI (Uniprot F7WD42), I-SceI (Uniprot P03882), I-AniI (Uniprot P03880), I-Dmol (Uniprot P21505), I-CreI (Uniprot P05725), I-TevI (Uniprot P13299), I-OnuI (Uniprot Q4VWW5), or I-Bmol (Uniprot Q9ANR6). In some embodiments, the meganuclease is naturally monomeric, e.g., I-SceI, I-TevI, or dimeric, e.g., I-CreI, in its functional form. For example, the LAGLIDADG meganucleases (SEQ ID NO: 37638) with a single copy of the LAGLIDADG motif (SEQ ID NO: 37638) generally form homodimers, whereas members with two copies of the LAGLIDADG motif (SEQ ID NO: 37638) are generally found as monomers. In some embodiments, a meganuclease that normally forms as a dimer is expressed as a fusion, e.g., the two subunits are expressed as a single ORF and, optionally, connected by a linker, e.g., an I-CreI dimer fusion (Rodriguez-Fornes et al. Gene Therapy 2020; incorporated by reference herein in its entirety). In some embodiments, a meganuclease, or a functional fragment thereof, is altered to favor nickase activity for one strand of a double-stranded DNA molecule, e.g., I-SceI (K122I and/or K223I) (Niu et al. J Mol Biol 2008), I-AniI (K227M) (McConnell Smith et al. PNAS 2009), I-DmoI (Q42A and/or K120M) (Molina et al. J Biol Chem 2015). In some embodiments, a meganuclease or functional fragment thereof possessing this preference for single-strand cleavage is used as an endonuclease domain, e.g., with nickase activity. In some embodiments, an endonuclease domain comprises a meganuclease, or a functional fragment thereof, which naturally targets or is engineered to target a safe harbor site, e.g., an I-CreI targeting SH6 site (Rodriguez-Fornes et al., supra). In some embodiments, an endonuclease domain comprises a meganuclease, or a functional fragment thereof, with a sequence tolerant catalytic domain, e.g., I-TevI recognizing the minimal motif CNNNG (Kleinstiver et al. PNAS 2012). In some embodiments, a target sequence tolerant catalytic domain is fused to a DNA binding domain, e.g., to direct activity, e.g., by fusing I-TevI to: (i) zinc fingers to create Tev-ZFEs (Kleinstiver et al. PNAS 2012), (ii) other meganucleases to create MegaTevs (Wolfs et al. Nucleic Acids Res 2014), and/or (iii) Cas9 to create TevCas9 (Wolfs et al. PNAS 2016).

In some embodiments, the endonuclease domain comprises a restriction enzyme, e.g., a Type IIS or Type IIP restriction enzyme. In some embodiments, the endonuclease domain comprises a Type IIS restriction enzyme, e.g., FokI, or a fragment or variant thereof. In some embodiments, the endonuclease domain comprises a Type IIP restriction enzyme, e.g., PvuII, or a fragment or variant thereof. In some embodiments, a dimeric restriction enzyme is expressed as a fusion such that it functions as a single chain, e.g., a FokI dimer fusion (Minczuk et al. Nucleic Acids Res 36 (12): 3926-3938 (2008)).

The use of additional endonuclease domains is described, for example, in Guha and Edgell Int J Mol Sci 18 (22): 2565 (2017), which is incorporated herein by reference in its entirety.

In some embodiments, a heterologous gene modifying polypeptide comprises a modification to an endonuclease domain, e.g., relative to a wild-type Cas protein. In some embodiments, the endonuclease domain comprises an addition, deletion, replacement, or modification to the amino acid sequence of the wild-type Cas protein. In some embodiments, the endonuclease domain is modified to include a heterologous functional domain that binds specifically to and/or induces endonuclease cleavage of a target nucleic acid (e.g., DNA) sequence of interest. In some embodiments, the endonuclease domain comprises a zinc finger. In embodiments, the endonuclease domain comprising the Cas domain is associated with a guide RNA (gRNA), e.g., as described herein. In some embodiments, the endonuclease domain is modified to include a functional domain that does not target a specific target nucleic acid (e.g., DNA) sequence. In embodiments, the endonuclease domain comprises a Fok1 domain.

In some embodiments, the endonuclease domain is associated with the target dsDNA in vitro at a frequency at least about 5-fold or 10-fold higher than with a scrambled dsDNA. In some embodiments, the endonuclease domain is associated with the target dsDNA in vitro at a frequency at least about 5-fold or 10-fold higher than with a scrambled dsDNA, e.g., in a cell (e.g., a HEK293T cell). In some embodiments, the frequency of association between the endonuclease domain and the target DNA or scrambled DNA is measured by ChIP-seq, e.g., as described in He and Pu (2010) Curr. Protoc Mol Biol Chapter 21 (incorporated by reference herein in its entirety).

In some embodiments, the endonuclease domain can catalyze the formation of a nick at a target sequence, e.g., to an increase of at least about 5-fold or 10-fold relative to a non-target sequence (e.g., relative to any other genomic sequence in the genome of the target cell). In some embodiments, the level of nick formation is determined using NickSeq, e.g., as described in Elacqua et al. (2019) bioRxiv doi.org/10.1101/867937 (incorporated herein by reference in its entirety).

In some embodiments, the endonuclease domain is capable of nicking DNA in vitro. In embodiments, the nick results in an exposed base. In embodiments, the exposed base can be detected using a nuclease sensitivity assay, e.g., as described in Chaudhry and Weinfeld (1995) Nucleic Acids Res 23 (19): 3805-3809 (incorporated by reference herein in its entirety). In embodiments, the level of exposed bases (e.g., detected by the nuclease sensitivity assay) is increased by at least 10%, 50%, or more relative to a reference endonuclease domain. In some embodiments, the reference endonuclease domain is an endonuclease domain from Cas9 of S. pyogenes.

In some embodiments, the endonuclease domain is capable of nicking DNA in a cell. In embodiments, the endonuclease domain is capable of nicking DNA in a HEK293T cell. In embodiments, an unrepaired nick that undergoes replication in the absence of Rad51 results in increased NHEJ rates at the site of the nick, which can be detected, e.g., by using a Rad51 inhibition assay, e.g., as described in Bothmer et al. (2017) Nat Commun 8:13905 (incorporated by reference herein in its entirety). In embodiments, NHEJ rates are increased above 0-5%. In embodiments, NHEJ rates are increased to 20-70% (e.g., between 30%-60% or 40-50%), e.g., upon Rad51 inhibition.

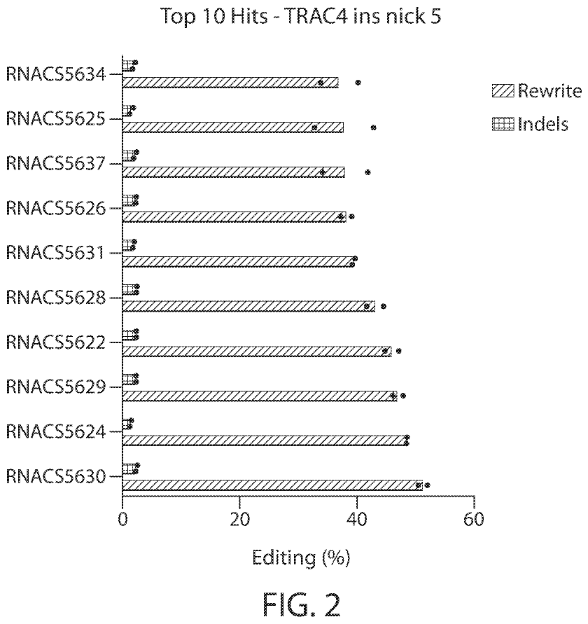

In some embodiments, the endonuclease domain releases the target after cleavage. In some embodiments, release of the target is indicated indirectly by assessing for multiple turnovers by the enzyme, e.g., as described in Yourik at al. RNA 25 (1): 35-44 (2019) (incorporated herein by reference in its entirety) and shown in FIG. 2. In some embodiments, the kₑₓₚ of an endonuclease domain is 1×10⁻³-1×10⁻⁵ min-1 as measured by such methods.

In some embodiments, the endonuclease domain has a catalytic efficiency (k_{cat}/Kₘ) greater than about 1×10⁸ s⁻¹ M⁻¹ in vitro. In embodiments, the endonuclease domain has a catalytic efficiency greater than about 1×10⁵, 1×10⁶, 1×10⁷, or 1×10⁸, s⁻¹ M⁻¹ in vitro. In embodiments, catalytic efficiency is determined as described in Chen et al. (2018) Science 360 (6387): 436-439 (incorporated herein by reference in its entirety). In some embodiments, the endonuclease domain has a catalytic efficiency (k_{cat}/Kₘ) greater than about 1×10⁸ s⁻¹ M⁻¹ in cells. In embodiments, the endonuclease domain has a catalytic efficiency greater than about 1×10⁵, 1×10⁶, 1×10⁷, or 1×10⁸ s⁻¹ M⁻¹ in cells.

### Heterologous Gene Modifying Polypeptides Comprising Cas Domains

In some embodiments, a heterologous gene modifying polypeptide described herein comprises a Cas domain. In some embodiments, the Cas domain can direct the heterologous gene modifying polypeptide to a target site specified by a gRNA spacer, thereby modifying a target nucleic acid sequence in “cis”. In some embodiments, a heterologous gene modifying polypeptide is fused to a Cas domain. In some embodiments, a heterologous gene modifying polypeptide comprises a CRISPR/Cas domain (also referred to herein as a CRISPR-associated protein). In some embodiments, a CRISPR/Cas domain comprises a protein involved in the clustered regulatory interspaced short palindromic repeat (CRISPR) system, e.g., a Cas protein, and optionally binds a guide RNA, e.g., single guide RNA (sgRNA).

CRISPR systems are adaptive defense systems originally discovered in bacteria and archaea. CRISPR systems use RNA-guided nucleases termed CRISPR-associated or “Cas” endonucleases (e.g., Cas9 or Cpf1) to cleave foreign DNA. For example, in a typical CRISPR-Cas system, an endonuclease is directed to a target nucleotide sequence (e.g., a site in the genome that is to be sequence-edited) by sequence-specific, non-coding “guide RNAs” that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “spacer” sequence, a typically about 20-nucleotide RNA sequence that corresponds to a target DNA sequence (“protospacer”). In the wild-type system, and in some engineered systems, crRNA also contains a region that binds to the tracrRNA to form a partially double-stranded structure that is cleaved by RNase III, resulting in a crRNA/tracrRNA hybrid molecule. A crRNA/tracrRNA hybrid then directs the Cas endonuclease to recognize and cleave a target DNA sequence. A target DNA sequence is generally adjacent to a “protospacer adjacent motif” (“PAM”) that is specific for a given Cas endonuclease and required for cleavage activity at a target site matching the spacer of the crRNA. CRISPR endonucleases identified from various prokaryotic species have unique PAM sequence requirements, e.g., as listed for exemplary Cas enzymes in Table 7; examples of PAM sequences include 5′-NGG (Streptococcus pyogenes), 5′-NNAGAA (Streptococcus thermophilus CRISPR1), 5′-NGGNG (Streptococcus thermophilus CRISPR3), and 5′-NNNGATT (Neisseria meningitidis). Some endonucleases, e.g., Cas9 endonucleases, are associated with G-rich PAM sites, e.g., 5′-NGG, and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1-associated CRISPR arrays are processed into mature crRNAs without the requirement of a tracrRNA; in other words, a Cpf1 system, in some embodiments, comprises only Cpf1 nuclease and a crRNA to cleave a target DNA sequence. Cpf1 endonucleases, are typically associated with T-rich PAM sites, e.g., 5′-TTN. Cpf1 can also recognize a 5′-CTA PAM motif. Cpf1 typically cleaves a target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from a PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e.g., Zetsche et al. (2015) Cell, 163:759-771.

A variety of CRISPR associated (Cas) genes or proteins can be used in the technologies provided by the present disclosure and the choice of Cas protein will depend upon the particular conditions of the method. Specific examples of Cas proteins include class II systems including Cas1, Cas2, Cas3, Cas4, Cas5, Cas6, Cas7, Cas8, Cas9, Cas10, Cpf1, C2C1, or C2C3. In some embodiments, a Cas protein, e.g., a Cas9 protein, may be from any of a variety of prokaryotic species. In some embodiments a particular Cas protein, e.g., a particular Cas9 protein, is selected to recognize a particular protospacer-adjacent motif (PAM) sequence. In some embodiments, a DNA-binding domain or endonuclease domain includes a sequence targeting polypeptide, such as a Cas protein, e.g., Cas9. In certain embodiments a Cas protein, e.g., a Cas9 protein, may be obtained from a bacteria or archaea or synthesized using known methods. In certain embodiments, a Cas protein may be from a gram-positive bacteria or a gram-negative bacteria. In certain embodiments, a Cas protein may be from a Streptococcus (e.g., a S. pyogenes, or a S. thermophilus), a Francisella (e.g., an F. novicida), a Staphylococcus (e.g., an S. aureus), an Acidaminococcus (e.g., an Acidaminococcus sp. BV3L6), a Neisseria (e.g., an N. meningitidis), a Cryptococcus, a Corynebacterium, a Haemophilus, a Eubacterium, a Pasteurella, a Prevotella, a Veillonella, or a Marinobacter.

In some embodiments, a heterologous gene modifying polypeptide may comprise the amino acid sequence of SEQ ID NO: 4000 below, or a sequence having at least 70%, 75%, 80%, 85%, 90%, 95%, 96%, 97%, 98%, 99% identity thereto. In embodiments, the amino acid sequence of SEQ ID NO: 4000 below, or the sequence having at least 70%, 75%, 80%, 85%, 90%, 95%, 96%, 97%, 98%, 99% identity thereto, is positioned at the N-terminal end of the heterologous gene modifying polypeptide. In embodiments, the amino acid sequence of SEQ ID NO: 4000 below, or the sequence having at least 70%, 75%, 80%, 85%, 90%, 95%, 96%, 97%, 98%, 99% identity thereto, is positioned within 1, 2, 3, 4, 5, 6, 7, 8, 9, 10, 15, 20, 25, or 30 amino acids of the N-terminal end of the heterologous gene modifying polypeptide.

Exemplary N-terminal NLS-Cas9 domain
(SEQ ID NO: 4000)
MPAAKRVKLDGGDKKYSIGLDIGTNSVGWAVITDEYKVPSKKFKVLGNTD
RHSIKKNLIGALLFDSGETAEATRLKRTARRRYTRRKNRICYLQEIFSNE
MAKVDDSFFHRLEESFLVEEDKKHERHPIFGNIVDEVAYHEKYPTIYHLR
KKLVDSTDKADLRLIYLALAHMIKFRGHFLIEGDLNPDNSDVDKLFIQLV
QTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADL
FLAAKNLSDAILLSDILRVNTEITKAPLSASMIKRYDEHHQDLTLLKALV
RQQLPEKYKEIFFDQSKNGYAGYIDGGASQEEFYKFIKPILEKMDGTEEL
LVKLNREDLLRKQRTFDNGSIPHQIHLGELHAILRRQEDFYPFLKDNREK
IEKILTFRIPYYVGPLARGNSRFAWMTRKSEETITPWNFEEVVDKGASAQ
SFIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFNA
SLGTYHDLLKIIKDKDFLDNEENEDILEDIVLTLTLFEDREMIEERLKTY
AHLFDDKVMKQLKRRRYTGWGRLSRKLINGIRDKQSGKTILDFLKSDGFA
NRNFMQLIHDDSLTFKEDIQKAQVSGQGDSLHEHIANLAGSPAIKKGILQ
TVKVVDELVKVMGRHKPENIVIEMARENQTTQKGQKNSRERMKRIEEGIK
ELGSQILKEHPVENTQLQNEKLYLYYLQNGRDMYVDQELDINRLSDYDVD
HIVPQSFLKDDSIDNKVLTRSDKARGKSDNVPSEEVVKKMKNYWRQLLNA
KLITQRKFDNLTKAERGGLSELDKAGFIKRQLVETRQITKHVAQILDSRM
NTKYDENDKLIREVKVITLKSKLVSDFRKDFQFYKVREINNYHHAHDAYL
NAVVGTALIKKYPKLESEFVYGDYKVYDVRKMIAKSEQEIGKATAKYFFY
SNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTV
AYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLA
SHYEKLKGSPEDNEQKQLFVEQHKHYLDEIIEQISEFSKRVILADANLDK
VLSAYNKHRDKPIREQAENIIHLFTLTNLGAPAAFKYFDTTIDRKRYTST
KEVLDATLIHQSITGLYETRIDLSQLGGDGG

In some embodiments, a heterologous gene modifying polypeptide may comprise the amino acid sequence of SEQ ID NO: 4001 below, or a sequence having at least 70%, 75%, 80%, 85%, 90%, 95%, 96%, 97%, 98%, 99% identity thereto. In embodiments, the amino acid sequence of SEQ ID NO: 4001 below, or the sequence having at least 70%, 75%, 80%, 85%, 90%, 95%, 96%, 97%, 98%, 99% identity thereto, is positioned at the C-terminal end of the heterologous gene modifying polypeptide. In embodiments, the amino acid sequence of SEQ ID NO: 4001 below, or the sequence having at least 70%, 75%, 80%, 85%, 90%, 95%, 96%, 97%, 98%, 99% identity thereto, is positioned within 1, 2, 3, 4, 5, 6, 7, 8, 9, 10, 15, 20, 25, or 30 amino acids of the C-terminal end of the heterologous gene modifying polypeptide.

Exemplary C-terminal sequence
(SEQ ID NO: 4001)
AGKRTADGSEFEKRTADGSEFESPKKKAKVE
Exemplary benchmarking sequence
(SEQ ID NO: 4002)
MPAAKRVKLDGGDKKYSIGLDIGTNSVGWAVITDEYKVPSKKFKVLGNTD
RHSIKKNLIGALLFDSGETAEATRLKRTARRRYTRRKNRICYLQEIFSNE
MAKVDDSFFHRLEESFLVEEDKKHERHPIFGNIVDEVAYHEKYPTIYHLR
KKLVDSTDKADLRLIYLALAHMIKFRGHFLIEGDLNPDNSDVDKLFIQLV
QTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADL
FLAAKNLSDAILLSDILRVNTEITKAPLSASMIKRYDEHHQDLTLLKALV
RQQLPEKYKEIFFDQSKNGYAGYIDGGASQEEFYKFIKPILEKMDGTEEL
LVKLNREDLLRKQRTFDNGSIPHQIHLGELHAILRRQEDFYPFLKDNREK
IEKILTFRIPYYVGPLARGNSRFAWMTRKSEETITPWNFEEVVDKGASAQ
SFIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFNA
SLGTYHDLLKIIKDKDFLDNEENEDILEDIVLTLTLFEDREMIEERLKTY
AHLFDDKVMKQLKRRRYTGWGRLSRKLINGIRDKQSGKTILDFLKSDGFA
NRNFMQLIHDDSLTFKEDIQKAQVSGQGDSLHEHIANLAGSPAIKKGILQ
TVKVVDELVKVMGRHKPENIVIEMARENQTTQKGQKNSRERMKRIEEGIK
ELGSQILKEHPVENTQLQNEKLYLYYLQNGRDMYVDQELDINRLSDYDVD
HIVPQSFLKDDSIDNKVLTRSDKARGKSDNVPSEEVVKKMKNYWRQLLNA
KLITQRKFDNLTKAERGGLSELDKAGFIKRQLVETRQITKHVAQILDSRM
NTKYDENDKLIREVKVITLKSKLVSDFRKDFQFYKVREINNYHHAHDAYL
NAVVGTALIKKYPKLESEFVYGDYKVYDVRKMIAKSEQEIGKATAKYFFY
SNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTV
AYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLA
SHYEKLKGSPEDNEQKQLFVEQHKHYLDEIIEQISEFSKRVILADANLDK
VLSAYNKHRDKPIREQAENIIHLFTLTNLGAPAAFKYFDTTIDRKRYTST
KEVLDATLIHQSITGLYETRIDLSQLGGDGGSGGSSGGSSGSETPGTSES
ATPESSGGSSGGSSGGTLNIEDEYRLHETSKEPDVSLGSTWLSDFPQAWA
ETGGMGLAVRQAPLIIPLKATSTPVSIKQYPMSQEARLGIKPHIQRLLDQ
GILVPCQSPWNTPLLPVKKPGTNDYRPVQDLREVNKRVEDIHPTVPNPYN
LLSGLPPSHQWYTVLDLKDAFFCLRLHPTSQPLFAFEWRDPEMGISGQLT
WTRLPQGFKNSPTLFNEALHRDLADFRIQHPDLILLQYVDDLLLAATSEL
DCQQGTRALLQTLGNLGYRASAKKAQICQKQVKYLGYLLKEGQRWLTEAR
KETVMGQPTPKTPRQLREFLGKAGFCRLFIPGFAEMAAPLYPLTKPGTLF
NWGPDQQKAYQEIKQALLTAPALGLPDLTKPFELFVDEKQGYAKGVLTQK
LGPWRRPVAYLSKKLDPVAAGWPPCLRMVAAIAVLTKDAGKLTMGQPLVI
LAPHAVEALVKQPPDRWLSNARMTHYQALLLDTDRVQFGPVVALNPATLL
PLPEEGLQHNCLDILAEAHGTRPDLTDQPLPDADHTWYTDGSSLLQEGQR
KAGAAVTTETEVIWAKALPAGTSAQRAELIALTQALKMAEGKKLNVYTDS
RYAFATAHIHGEIYRRRGWLTSEGKEIKNKDEILALLKALFLPKRLSIIH
CPGHQKGHSAEARGNRMADQAARKAAITETPDTSTLLIENSSPSGGSKRT
ADGSEFEAGKRTADGSEFEKRTADGSEFESPKKKAKVE

In some embodiments, a heterologous gene modifying polypeptide may comprise a Cas domain as listed in Table 7 or 8, or a functional fragment thereof, or a sequence having at least 70%, 75%, 80%, 85%, 90%, 95%, 96%, 97%, 98%, 99% identity thereto.

TABLE 7
CRISPR/Cas Proteins, Species, and Mutations

| | | | # of | | Mutations to alter | Mutations to make |
|---|---|---|---|---|---|---|
| Name | Enzyme | Species | AAs | PAM | PAM recognition | catalytically dead |
| FnCas9 | Cas9 | Francisella | 1629 | 5'-NGG-3' | Wt | D11A/H969A/N995A |
| | | novicida | | | | |
| FnCas9 | Cas9 | Francisella | 1629 | 5'-YG-3' | E1369R/E1449H/R1556A | D11A/H969A/N995A |
| RHA | | novicida | | | | |
| SaCas9 | Cas9 | Staphylococcus | 1053 | 5'-NNGRRT- | Wt | D10A/H557A |
| | | aureus | | 3' | | |
| SaCas9 | Cas9 | Staphylococcus | 1053 | 5'-NNNRRT- | E782K/N968K/R1015H | D10A/H557A |
| KKH | | aureus | | 3' | | |
| SpCas9 | Cas9 | Streptococcus | 1368 | 5'-NGG-3' | Wt | D10A/D839A/H840A/N |
| | | pyogenes | | | | 863A |
| SpCas9 | Cas9 | Streptococcus | 1368 | 5'-NGA-3' | D1135V/R1335Q/T1337R | D10A/D839A/H840A/N |
| VQR | | pyogenes | | | | 863A |
| AsCpf1 | Cpf1 | Acidaminococcus | 1307 | 5'-TYCV-3' | S542R/K607R | E993A |
| RR | | sp. BV3L6 | | | | |
| AsCpf1 | Cpf1 | Acidaminococcus | 1307 | 5'-TATV-3' | S542R/K548V/N552R | E993A |
| RVR | | sp. BV3L6 | | | | |
| FnCpf1 | Cpf1 | Francisella | 1300 | 5'-NTTN-3' | Wt | D917A/E1006A/D1255 |
| | | novicida | | | | A |
| NmCas9 | Cas9 | Neisseria | 1082 | 5'- | Wt | D16A/D587A/H588A/N |
| | | meningitidis | | NNNGATT- | | 611A |
| | | | | 3 | | |

TABLE 8
Amino Acid Sequences of CRISPR/Cas Proteins, Species, and Mutations

| | Parental | | SEQ ID | | | |
|---|---|---|---|---|---|---|
| Variant | Host(s) | Protein Sequence | NO: | Nickase (HNH) | Nickase(HNH) | Nickase(RuvC) |
| Nme2Cas9 | Neisseria | MAAFKPNPINYILGLDIGIASVGWAMVEIDEEENPIRLIDLGVRVFERAEVPK | 9,001 | N611A | H588A | D16A |
| | meningitidis | TGDSLAMARRLARSVRRLTRRRAHRLLRARRLLKREGVLQAADFDENGLIKS | | | | |
| | | LPNTPWQLRAAALDRKLTPLEWSAVLLHLIKHRGYLSQRKNEGETADKELG | | | | |
| | | ALLKGVANNAHALQTGDFRTPAELALNKFEKESGHIRNQRGDYSHTFSRKD | | | | |
| | | LQAELILLFEKQKEFGNPHVSGGLKEGIETLLMTQRPALSGDAVQKMLGHCT | | | | |
| | | FEPAEPKAAKNTYTAERFIWLTKLNNLRILEQGSERPLTDTERATLMDEPYRK | | | | |
| | | SKLTYAQARKLLGLEDTAFFKGLRYGKDNAEASTLMEMKAYHAISRALEKEG | | | | |
| | | LKDKKSPLNLSSELQDEIGTAFSLFKTDEDITGRLKDRVQPEILEALLKHISFDKF | | | | |
| | | VQISLKALRRIVPLMEQGKRYDEACAEIYGDHYGKKNTEEKIYLPPIPADEIRN | | | | |
| | | PVVLRALSQARKVINGVVRRYGSPARIHIETAREVGKSFKDRKEIEKRQEENR | | | | |
| | | KDREKAAAKFREYFPNFVGEPKSKDILKLRLYEQQHGKCLYSGKEINLVRLNE | | | | |
| | | KGYVEIDHALPFSRTWDDSFNNKVLVLGSENQNKGNQTPYEYFNGKDNSR | | | | |
| | | EWQEFKARVETSRFPRSKKQRILLQKFDEDGFKECNLNDTRYVNRFLCQFVA | | | | |
| | | DHILLTGKGKRRVFASNGQITNLLRGFWGLRKVRAENDRHHALDAVVVACS | | | | |
| | | TVAMQQKITRFVRYKEMNAFDGKTIDKETGKVLHQKTHFPQPWEFFAQEV | | | | |
| | | MIRVFGKPDGKPEFEEADTPEKLRTLLAEKLSSRPEAVHEYVTPLFVSRAPNR | | | | |
| | | KMSGAHKDTLRSAKRFVKHNEKISVKRVWLTEIKLADLENMVNYKNGREIEL | | | | |
| | | YEALKARLEAYGGNAKQAFDPKDNPFYKKGGQLVKAVRVEKTQESGVLLNK | | | | |
| | | KNAYTIADNGDMVRVDVFCKVDKKGKNQYFIVPIYAWQVAENILPDIDCKG | | | | |
| | | YRIDDSYTFCFSLHKYDLIAFQKDEKSKVEFAYYINCDSSNGRFYLAWHDKGS | | | | |
| | | KEQQFRISTQNLVLIQKYQVNELGKEIRPCRLKKRPPVR | | | | |
| PpnCas9 | Pasteurella | MQNNPLNYILGLDLGIASIGWAVVEIDEESSPIRLIDVGVRTFERAEVAKTGE | 9,002 | N605A | H582A | D13A |
| | pneumotropica | SLALSRRLARSSRRLIKRRAERLKKAKRLLKAEKILHSIDEKLPINVWQLRVKGL | | | | |
| | | KEKLERQEWAAVLLHLSKHRGYLSQRKNEGKSDNKELGALLSGIASNHQML | | | | |
| | | QSSEYRTPAEIAVKKFQVEEGHIRNQRGSYTHTFSRLDLLAEMELLFQRQAEL | | | | |
| | | GNSYTSTTLLENLTALLMWQKPALAGDAILKMLGKCTFEPSEYKAAKNSYSA | | | | |
| | | ERFVWLTKLNNLRILENGTERALNDNERFALLEQPYEKSKLTYAQVRAMLAL | | | | |
| | | SDNAIFKGVRYLGEDKKTVESKTTLIEMKFYHQIRKTLGSAELKKEWNELKGN | | | | |
| | | SDLLDEIGTAFSLYKTDDDICRYLEGKLPERVLNALLENLNFDKFIQLSLKALHQ | | | | |
| | | ILPLMLQGQRYDEAVSAIYGDHYGKKSTETTRLLPTIPADEIRNPVVLRTLTQA | | | | |
| | | RKVINAVVRLYGSPARIHIETAREVGKSYQDRKKLEKQQEDNRKQRESAVKK | | | | |
| | | FKEMFPHFVGEPKGKDILKMRLYELQQAKCLYSGKSLELHRLLEKGYVEVDH | | | | |
| | | ALPFSRTWDDSFNNKVLVLANENQNKGNLTPYEWLDGKNNSERWQHFVV | | | | |
| | | RVQTSGFSYAKKQRILNHKLDEKGFIERNLNDTRYVARFLCNFIADNMLLVG | | | | |
| | | KGKRNVFASNGQITALLRHRWGLQKVREQNDRHHALDAVVVACSTVAMQ | | | | |
| | | QKITRFVRYNEGNVFSGERIDRETGEIIPLHFPSPWAFFKENVEIRIFSENPKLE | | | | |
| | | LENRLPDYPQYNHEWVQPLFVSRMPTRKMTGQGHMETVKSAKRLNEGLS | | | | |
| | | VLKVPLTQLKLSDLERMVNRDREIALYESLKARLEQFGNDPAKAFAEPFYKKG | | | | |
| | | GALVKAVRLEQTQKSGVLVRDGNGVADNASMVRVDVFTKGGKYFLVPIYT | | | | |
| | | WQVAKGILPNRAATQGKDENDWDIMDEMATFQFSLCQNDLIKLVTKKKTI | | | | |
| | | FGYFNGLNRATSNINIKEHDLDKSKGKLGIYLEVGVKLAISLEKYQVDELGKNI | | | | |
| | | RPCRPTKRQHVR | | | | |
| SauCas9 | Staphylococcus | MKRNYILGLDIGITSVGYGIIDYETRDVIDAGVRLFKEANVENNEGRRSKRGA | 9,003 | N580A | H557A | D10A |
| | aureus | RRLKRRRRHRIQRVKKLLFDYNLLTDHSELSGINPYEARVKGLSQKLSEEEFSA | | | | |
| | | ALLHLAKRRGVHNVNEVEEDTGNELSTKEQISRNSKALEEKYVAELQLERLKK | | | | |
| | | DGEVRGSINRFKTSDYVKEAKQLLKVQKAYHQLDQSFIDTYIDLLETRRTYYE | | | | |
| | | GPGEGSPFGWKDIKEWYEMLMGHCTYFPEELRSVKYAYNADLYNALNDLN | | | | |
| | | NLVITRDENEKLEYYEKFQUIENVFKQKKKPTLKQIAKEILVNEEDIKGYRVTST | | | | |
| | | GKPEFTNLKVYHDIKDITARKEIIENAELLDQIAKILTIYQSSEDIQEELTNLNSE | | | | |
| | | LTQEEIEQISNLKGYTGTHNLSLKAINLILDELWHTNDNQIAIFNRLKLVPKKV | | | | |
| | | DLSQQKEIPTTLVDDFILSPVVKRSFIQSIKVINAIIKKYGLPNDIIIELAREKNSK | | | | |
| | | DAQKMINEMQKRNRQTNERIEEIIRTTGKENAKYLIEKIKLHDMQEGKCLYS | | | | |
| | | LEAIPLEDLLNNPFNYEVDHIIPRSVSFDNSFNNKVLVKQEENSKKGNRTPFQ | | | | |
| | | YLSSSDSKISYETFKKHILNLAKGKGRISKTKKEYLLEERDINRFSVQKDFINRNL | | | | |
| | | VDTRYATRGLMNLLRSYFRVNNLDVKVKSINGGFTSFLRRKWKFKKERNKG | | | | |
| | | YKHHAEDALIIANADFIFKEWKKLDKAKKVMENQMFEEKQAESMPEIETEQ | | | | |
| | | EYKEIFITPHQIKHIKDFKDYKYSHRVDKKPNRELINDTLYSTRKDDKGNTLIVN | | | | |
| | | NLNGLYDKDNDKLKKLINKSPEKLLMYHHDPQTYQKLKLIMEQYGDEKNPL | | | | |
| | | YKYYEETGNYLTKYSKKDNGPVIKKIKYYGNKLNAHLDITDDYPNSRNKVVKL | | | | |
| | | SLKPYRFDVYLDNGVYKFVTVKNLDVIKKENYYEVNSKCYEEAKKLKKISNQA | | | | |
| | | EFIASFYNNDLIKINGELYRVIGVNNDLLNRIEVNMIDITYREYLENMNDKRPP | | | | |
| | | RIIKTIASKTQSIKKYSTDILGNLYEVKSKKHPQIIKKG | | | | |
| SauCas9- | Staphylococcus | MKRNYILGLDIGITSVGYGIIDYETRDVIDAGVRLFKEANVENNEGRRSKRGA | 9,004 | N580A | H557A | D10A |
| KKH | aureus | RRLKRRRRHRIQRVKKLLFDYNLLTDHSELSGINPYEARVKGLSQKLSEEEFSA | | | | |
| | | ALLHLAKRRGVHNVNEVEEDTGNELSTKEQISRNSKALEEKYVAELQLERLKK | | | | |
| | | DGEVRGSINRFKTSDYVKEAKQLLKVQKAYHQLDQSFIDTYIDLLETRRTYYE | | | | |
| | | GPGEGSPFGWKDIKEWYEMLMGHCTYFPEELRSVKYAYNADLYNALNDLN | | | | |
| | | NLVITRDENEKLEYYEKFQIIENVFKQKKKPTLKQIAKEILVNEEDIKGYRVTST | | | | |
| | | GKPEFTNLKVYHDIKDITARKEIIENAELLDQIAKILTIYQSSEDIQEELTNLNSE | | | | |
| | | LTQEEIEQISNLKGYTGTHNLSLKAINLILDELWHTNDNQIAIFNRLKLVPKKV | | | | |
| | | DLSQQKEIPTTLVDDFILSPVVKRSFIQSIKVINAIIKKYGLPNDIIIELAREKNSK | | | | |
| | | DAQKMINEMQKRNRQTNERIEEIIRTTGKENAKYLIEKIKLHDMQEGKCLYS | | | | |
| | | LEAIPLEDLLNNPFNYEVDHIIPRSVSFDNSFNNKVLVKQEENSKKGNRTPFQ | | | | |
| | | YLSSSDSKISYETFKKHILNLAKGKGRISKTKKEYLLEERDINRFSVQKDFINRNL | | | | |
| | | VDTRYATRGLMNLLRSYFRVNNLDVKVKSINGGFTSFLRRKWKFKKERNKG | | | | |
| | | YKHHAEDALIIANADFIFKEWKKLDKAKKVMENQMFEEKQAESMPEIETEQ | | | | |
| | | EYKEIFITPHQIKHIKDFKDYKYSHRVDKKPNRKLINDTLYSTRKDDKGNTLIV | | | | |
| | | NNLNGLYDKDNDKLKKLINKSPEKLLMYHHDPQTYQKLKLIMEQYGDEKNP | | | | |
| | | LYKYYEETGNYLTKYSKKDNGPVIKKIKYYGNKLNAHLDITDDYPNSRNKVVK | | | | |
| | | LSLKPYRFDVYLDNGVYKFVTVKNLDVIKKENYYEVNSKCYEEAKKLKKISNQ | | | | |
| | | AEFIASFYKNDLIKINGELYRVIGVNNDLLNRIEVNMIDITYREYLENMNDKRP | | | | |
| | | PHIIKTIASKTQSIKKYSTDILGNLYEVKSKKHPQIIKKG | | | | |
| SauriCas9 | Staphylococcus | MQENQQKQNYILGLDIGITSVGYGLIDSKTREVIDAGVRLFPEADSENNSNR | 9,005 | N588A | H565A | D15A |
| | auricularis | RSKRGARRLKRRRIHRLNRVKDLLADYQMIDLNNVPKSTDPYTIRVKGLREPL | | | | |
| | | TKEEFAIALLHIAKRRGLHNISVSMGDEEQDNELSTKQQLQKNAQQLQDKY | | | | |
| | | VCELQLERLTNINKVRGEKNRFKTEDFVKEVKQLCETQRQYHNIDDQFIQQY | | | | |
| | | IDLVSTRREYFEGPGNGSPYGWDGDLLKWYEKLMGRCTYFPEELRSVKYAYS | | | | |
| | | ADLFNALNDLNNLVVTRDDNPKLEYYEKYHIIENVFKQKKNPTLKQIAKEIGV | | | | |
| | | QDYDIRGYRITKSGKPQFTSFKLYHDLKNIFEQAKYLEDVEMLDEIAKILTIYQ | | | | |
| | | DEISIKKALDQLPELLTESEKSQIAQLTGYTGTHRLSLKCIHIVIDELWESPENQ | | | | |
| | | MEIFTRLNLKPKKVEMSEIDSIPTTLVDEFILSPVVKRAFIQSIKVINAVINRFGL | | | | |
| | | PEDIIIELAREKNSKDRRKFINKLQKQNEATRKKIEQLLAKYGNTNAKYMIEKI | | | | |
| | | KLHDMQEGKCLYSLEAIPLEDLLSNPTHYEVDHIIPRSVSFDNSLNNKVLVKQ | | | | |
| | | SENSKKGNRTPYQYLSSNESKISYNQFKQHILNLSKAKDRISKKKRDMLLEER | | | | |
| | | DINKFEVQKEFINRNLVDTRYATRELSNLLKTYFSTHDYAVKVKTINGGFTNH | | | | |
| | | LRKVWDFKKHRNHGYKHHAEDALVIANADFLFKTHKALRRTDKILEQPGLE | | | | |
| | | VNDTTVKVDTEEKYQELFETPKQVKNIKQFRDFKYSHRVDKKPNRQLINDTL | | | | |
| | | YSTREIDGETYVVQTLKDLYAKDNEKVKKLFTERPQKILMYQHDPKTFEKLM | | | | |
| | | TILNQYAEAKNPLAAYYEDKGEYVTKYAKKGNGPAIHKIKYIDKKLGSYLDVS | | | | |
| | | NKYPETQNKLVKLSLKSFRFDIYKCEQGYKMVSIGYLDVLKKDNYYYIPKDKYE | | | | |
| | | AEKQKKKIKESDLFVGSFYYNDLIMYEDELFRVIGVNSDINNLVELNMVDITY | | | | |
| | | KDFCEVNNVTGEKRIKKTIGKRVVLIEKYTTDILGNLYKTPLPKKPQLIFKRGEL | | | | |
| SauriCas9- | Staphylococcus | MQENQQKQNYILGLDIGITSVGYGLIDSKTREVIDAGVRLFPEADSENNSNR | 9,006 | N588A | H565A | D15A |
| KKH | auricularis | RSKRGARRLKRRRIHRLNRVKDLLADYQMIDLNNVPKSTDPYTIRVKGLREPL | | | | |
| | | TKEEFAIALLHIAKRRGLHNISVSMGDEEQDNELSTKQQLQKNAQQLQDKY | | | | |
| | | VCELQLERLTNINKVRGEKNRFKTEDFVKEVKQLCETQRQYHNIDDQFIQQY | | | | |
| | | IDLVSTRREYFEGPGNGSPYGWDGDLLKWYEKLMGRCTYFPEELRSVKYAYS | | | | |
| | | ADLFNALNDLNNLVVTRDDNPKLEYYEKYHIIENVFKQKKNPTLKQIAKEIGV | | | | |
| | | QDYDIRGYRITKSGKPQFTSFKLYHDLKNIFEQAKYLEDVEMLDEIAKILTIYQ | | | | |
| | | DEISIKKALDQLPELLTESEKSQIAQLTGYTGTHRLSLKCIHIVIDELWESPENQ | | | | |
| | | MEIFTRLNLKPKKVEMSEIDSIPTTLVDEFILSPVVKRAFIQSIKVINAVINRFGL | | | | |
| | | PEDIIIELAREKNSKDRRKFINKLQKQNEATRKKIEQLLAKYGNTNAKYMIEKI | | | | |
| | | KLHDMQEGKCLYSLEAIPLEDLLSNPTHYEVDHIIPRSVSFDNSLNNKVLVKQ | | | | |
| | | SENSKKGNRTPYQYLSSNESKISYNQFKQHILNLSKAKDRISKKKRDMLLEER | | | | |
| | | DINKFEVQKEFINRNLVDTRYATRELSNLLKTYFSTHDYAVKVKTINGGFTNH | | | | |
| | | LRKVWDFKKHRNHGYKHHAEDALVIANADFLFKTHKALRRTDKILEQPGLE | | | | |
| | | VNDTTVKVDTEEKYQELFETPKQVKNIKQFRDFKYSHRVDKKPNRKLINDTL | | | | |
| | | YSTREIDGETYVVQTLKDLYAKDNEKVKKLFTERPQKILMYQHDPKTFEKLM | | | | |
| | | TILNQYAEAKNPLAAYYEDKGEYVTKYAKKGNGPAIHKIKYIDKKLGSYLDVS | | | | |
| | | NKYPETQNKLVKLSLKSFRFDIYKCEQGYKMVSIGYLDVLKKDNYYYIPKDKYE | | | | |
| | | AEKQKKKIKESDLFVGSFYKNDLIMYEDELFRVIGVNSDINNLVELNMVDITY | | | | |
| | | KDFCEVNNVTGEKHIKKTIGKRVVLIEKYTTDILGNLYKTPLPKKPQLIFKRGEL | | | | |
| ScaCas9- | Streptococcus | MEKKYSIGLDIGTNSVGWAVITDDYKVPSKKFKVLGNTNRKSIKKNLMGALL | 9,007 | N872A | H849A | D10A |
| Sc++ | canis | FDSGETAEATRLKRTARRRYTRRKNRIRYLQEIFANEMAKLDDSFFQRLEESF | | | | |
| | | LVEEDKKNERHPIFGNLADEVAYHRNYPTIYHLRKKLADSPEKADLRLIYLALA | | | | |
| | | HIIKFRGHFLIEGKLNAENSDVAKLFYQLIQTYNQLFEESPLDEIEVDAKGILSA | | | | |
| | | RLSKSKRLEKLIAVFPNEKKNGLFGNIIALALGLTPNFKSNFDLTEDAKLQLSKD | | | | |
| | | TYDDDLDELLGQIGDQYADLFSAAKNLSDAILLSDILRSNSEVTKAPLSASMV | | | | |
| | | KRYDEHHQDLALLKTLVRQQFPEKYAEIFKDDTKNGYAGYVGADKKLRKRS | | | | |
| | | GKLATEEEFYKFIKPILEKMDGAEELLAKLNRDDLLRKQRTFDNGSIPHQIHLK | | | | |
| | | ELHAILRRQEEFYPFLKENREKIEKILTFRIPYYVGPLARGNSRFAWLTRKSEEA | | | | |
| | | ITPWNFEEVVDKGASAQSFIERMTNFDEQLPNKKVLPKHSLLYEYFTVYNEL | | | | |
| | | TKVKYVTERMRKPEFLSGEQKKAIVDLLFKTNRKVTVKQLKEDYFKKIECFDS | | | | |
| | | VEIIGVEDRFNASLGTYHDLLKIIKDKDFLDNEENEDILEDIVLTLTLFEDREMIE | | | | |
| | | ERLKTYAHLFDDKVMKQLKRRHYTGWGRLSRKMINGIRDKQSGKTILDFLKS | | | | |
| | | DGFSNRNFMQLIHDDSLTFKEEIEKAQVSGQGDSLHEQIADLAGSPAIKKGIL | | | | |
| | | QTVKIVDELVKVMGHKPENIVIEMARENQTTTKGLQQSRERKKRIEEGIKELE | | | | |
| | | SQILKENPVENTQLQNEKLYLYYLQNGRDMYVDQELDINRLSDYDVDHIVP | | | | |
| | | QSFIKDDSIDNKVLTRSVENRGKSDNVPSEEVVKKMKNYWRQLLNAKLITQ | | | | |
| | | RKFDNLTKAERGGLSEADKAGFIKRQLVETRQITKHVARILDSRMNTKRDKN | | | | |
| | | DKPIREVKVITLKSKLVSDFRKDFQLYKVRDINNYHHAHDAYLNAVVGTALIK | | | | |
| | | KYPKLESEFVYGDYKVYDVRKMIAKSEQEIGKATAKRFFYSNIMNFFKTEVKL | | | | |
| | | ANGEIRKRPLIETNGETGEVVWNKEKDFATVRKVLAMPQVNIVKKTEVQTG | | | | |
| | | GFSKESILSKRESAKLIPRKKGWDTRKYGGFGSPTVAYSILVVAKVEKGKAKKL | | | | |
| | | KSVKVLVGITIMEKGSYEKDPIGFLEAKGYKDIKKELIFKLPKYSLFELENGRRR | | | | |
| | | MLASAKELQKANELVLPQHLVRLLYYTQNISATTGSNNLGYIEQHREEFKEIF | | | | |
| | | EKIIDFSEKYILKNKVNSNLKSSFDEQFAVSDSILLSNSFVSLLKYTSFGASGGFT | | | | |
| | | FLDLDVKQGRLRYQTVTEVLDATLIYQSITGLYETRTDLSQLGGD | | | | |
| SpyCas9 | Streptococcus | MDKKYSIGLDIGTNSVGWAVITDEYKVPSKKFKVLGNTDRHSIKKNLIGALLF | 9,008 | N863A | H840A | D10A |
| | pyogenes | DSGETAEATRLKRTARRRYTRRKNRICYLQEIFSNEMAKVDDSFFHRLEESFL | | | | |
| | | VEEDKKHERHPIFGNIVDEVAYHEKYPTIYHLRKKLVDSTDKADLRLIYLALAH | | | | |
| | | MIKFRGHFLIEGDLNPDNSDVDKLFIQLVQTYNQLFEENPINASGVDAKAILS | | | | |
| | | ARLSKSRRLENLIAQLPGEKKNGLFGNLIALSLGLTPNFKSNFDLAEDAKLQLS | | | | |
| | | KDTYDDDLDNLLAQIGDQYADLFLAAKNLSDAILLSDILRVNTEITKAPLSAS | | | | |
| | | MIKRYDEHHQDLTLLKALVRQQLPEKYKEIFFDQSKNGYAGYIDGGASQEEF | | | | |
| | | YKFIKPILEKMDGTEELLVKLNREDLLRKQRTFDNGSIPHQIHLGELHAILRRQ | | | | |
| | | EDFYPFLKDNREKIEKILTFRIPYYVGPLARGNSRFAWMTRKSEETITPWNFE | | | | |
| | | EVVDKGASAQSFIERMTNFDKNLPNEKVLPKHSLLYEYFTVYNELTKVKYVTE | | | | |
| | | GMRKPAFLSGEQKKAIVDLLFKTNRKVTVKQLKEDYFKKIECFDSVEISGVED | | | | |
| | | RFNASLGTYHDLLKIIKDKDFLDNEENEDILEDIVLTLTLFEDREMIEERLKTYA | | | | |
| | | HLFDDKVMKQLKRRRYTGWGRLSRKLINGIRDKQSGKTILDFLKSDGFANR | | | | |
| | | NFMQLIHDDSLTFKEDIQKAQVSGQGDSLHEHIANLAGSPAIKKGILQTVKV | | | | |
| | | VDELVKVMGRHKPENIVIEMARENQTTQKGQKNSRERMKRIEEGIKELGSQ | | | | |
| | | ILKEHPVENTQLQNEKLYLYYLQNGRDMYVDQELDINRLSDYDVDHIVPQSF | | | | |
| | | LKDDSIDNKVLTRSDKNRGKSDNVPSEEVVKKMKNYWRQLLNAKLITQRKF | | | | |
| | | DNLTKAERGGLSELDKAGFIKRQLVETRQITKHVAQILDSRMNTKYDENDKLI | | | | |
| | | REVKVITLKSKLVSDFRKDFQFYKVREINNYHHAHDAYLNAVVGTALIKKYPK | | | | |
| | | LESEFVYGDYKVYDVRKMIAKSEQEIGKATAKYFFYSNIMNFFKTEITLANGEI | | | | |
| | | RKRPLIETNGETGEIVWDKGRDFATVRKVLSMPQVNIVKKTEVQTGGFSKES | | | | |
| | | ILPKRNSDKLIARKKDWDPKKYGGFDSPTVAYSVLVVAKVEKGKSKKLKSVKE | | | | |
| | | LLGITIMERSSFEKNPIDFLEAKGYKEVKKDLIIKLPKYSLFELENGRKRMLASA | | | | |
| | | GELQKGNELALPSKYVNFLYLASHYEKLKGSPEDNEQKQLFVEQHKHYLDEII | | | | |
| | | EQISEFSKRVILADANLDKVLSAYNKHRDKPIREQAENIIHLFTLTNLGAPAAF | | | | |
| | | KYFDTTIDRKRYTSTKEVLDATLIHQSITGLYETRIDLSQLGGD | | | | |
| SpyCas9- | Streptococcus | MDKKYSIGLDIGTNSVGWAVITDEYKVPSKKFKVLGNTDRHSIKKNLIGALLF | 9,009 | N863A | H840A | D10A |
| NG | pyogenes | DSGETAEATRLKRTARRRYTRRKNRICYLQEIFSNEMAKVDDSFFHRLEESFL | | | | |
| | | VEEDKKHERHPIFGNIVDEVAYHEKYPTIYHLRKKLVDSTDKADLRLIYLALAH | | | | |
| | | MIKFRGHFLIEGDLNPDNSDVDKLFIQLVQTYNQLFEENPINASGVDAKAILS | | | | |
| | | ARLSKSRRLENLIAQLPGEKKNGLFGNLIALSLGLTPNFKSNFDLAEDAKLQLS | | | | |
| | | KDTYDDDLDNLLAQIGDQYADLFLAAKNLSDAILLSDILRVNTEITKAPLSAS | | | | |
| | | MIKRYDEHHQDLTLLKALVRQQLPEKYKEIFFDQSKNGYAGYIDGGASQEEF | | | | |
| | | YKFIKPILEKMDGTEELLVKLNREDLLRKQRTFDNGSIPHQIHLGELHAILRRQ | | | | |
| | | EDFYPFLKDNREKIEKILTFRIPYYVGPLARGNSRFAWMTRKSEETITPWNFE | | | | |
| | | EVVDKGASAQSFIERMTNFDKNLPNEKVLPKHSLLYEYFTVYNELTKVKYVTE | | | | |
| | | GMRKPAFLSGEQKKAIVDLLFKTNRKVTVKQLKEDYFKKIECFDSVEISGVED | | | | |
| | | RFNASLGTYHDLLKIIKDKDFLDNEENEDILEDIVLTLTLFEDREMIEERLKTYA | | | | |
| | | HLFDDKVMKQLKRRRYTGWGRLSRKLINGIRDKQSGKTILDFLKSDGFANR | | | | |
| | | NFMQLIHDDSLTFKEDIQKAQVSGQGDSLHEHIANLAGSPAIKKGILQTVKV | | | | |
| | | VDELVKVMGRHKPENIVIEMARENQTTQKGQKNSRERMKRIEEGIKELGSQ | | | | |
| | | ILKEHPVENTQLQNEKLYLYYLQNGRDMYVDQELDINRLSDYDVDHIVPQSF | | | | |
| | | LKDDSIDNKVLTRSDKNRGKSDNVPSEEVVKKMKNYWRQLLNAKLITQRKF | | | | |
| | | DNLTKAERGGLSELDKAGFIKRQLVETRQITKHVAQILDSRMNTKYDENDKLI | | | | |
| | | REVKVITLKSKLVSDFRKDFQFYKVREINNYHHAHDAYLNAVVGTALIKKYPK | | | | |
| | | LESEFVYGDYKVYDVRKMIAKSEQEIGKATAKYFFYSNIMNFFKTEITLANGEI | | | | |
| | | RKRPLIETNGETGEIVWDKGRDFATVRKVLSMPQVNIVKKTEVQTGGFSKES | | | | |
| | | IRPKRNSDKLIARKKDWDPKKYGGFVSPTVAYSVLVVAKVEKGKSKKLKSVKE | | | | |
| | | LLGITIMERSSFEKNPIDFLEAKGYKEVKKDLIIKLPKYSLFELENGRKRMLASA | | | | |
| | | RFLQKGNELALPSKYVNFLYLASHYEKLKGSPEDNEQKQLFVEQHKHYLDEII | | | | |
| | | EQISEFSKRVILADANLDKVLSAYNKHRDKPIREQAENIIHLFTLTNLGAPRAF | | | | |
| | | KYFDTTIDRKVYRSTKEVLDATLIHQSITGLYETRIDLSQLGGD | | | | |
| SpyCas9- | Streptococcus | MDKKYSIGLDIGTNSVGWAVITDEYKVPSKKFKVLGNTDRHSIKKNLIGALLF | 9,010 | N863A | H840A | D10A |
| SpRY | pyogenes | DSGETAERTRLKRTARRRYTRRKNRICYLQEIFSNEMAKVDDSFFHRLEESFL | | | | |
| | | VEEDKKHERHPIFGNIVDEVAYHEKYPTIYHLRKKLVDSTDKADLRLIYLALAH | | | | |
| | | MIKFRGHFLIEGDLNPDNSDVDKLFIQLVQTYNQLFEENPINASGVDAKAILS | | | | |
| | | ARLSKSRRLENLIAQLPGEKKNGLFGNLIALSLGLTPNFKSNFDLAEDAKLQLS | | | | |
| | | KDTYDDDLDNLLAQIGDQYADLFLAAKNLSDAILLSDILRVNTEITKAPLSAS | | | | |
| | | MIKRYDEHHQDLTLLKALVRQQLPEKYKEIFFDQSKNGYAGYIDGGASQEEF | | | | |
| | | YKFIKPILEKMDGTEELLVKLNREDLLRKQRTFDNGSIPHQIHLGELHAILRRQ | | | | |
| | | EDFYPFLKDNREKIEKILTFRIPYYVGPLARGNSRFAWMTRKSEETITPWNFE | | | | |
| | | EVVDKGASAQSFIERMTNFDKNLPNEKVLPKHSLLYEYFTVYNELTKVKYVTE | | | | |
| | | GMRKPAFLSGEQKKAIVDLLFKTNRKVTVKQLKEDYFKKIECFDSVEISGVED | | | | |
| | | RFNASLGTYHDLLKIIKDKDFLDNEENEDILEDIVLTLTLFEDREMIEERLKTYA | | | | |
| | | HLFDDKVMKQLKRRRYTGWGRLSRKLINGIRDKQSGKTILDFLKSDGFANR | | | | |
| | | NFMQLIHDDSLTFKEDIQKAQVSGQGDSLHEHIANLAGSPAIKKGILQTVKV | | | | |
| | | VDELVKVMGRHKPENIVIEMARENQTTQKGQKNSRERMKRIEEGIKELGSQ | | | | |
| | | ILKEHPVENTQLQNEKLYLYYLQNGRDMYVDQELDINRLSDYDVDHIVPQSF | | | | |
| | | LKDDSIDNKVLTRSDKNRGKSDNVPSEEVVKKMKNYWRQLLNAKLITQRKF | | | | |
| | | DNLTKAERGGLSELDKAGFIKRQLVETRQITKHVAQILDSRMNTKYDENDKLI | | | | |
| | | REVKVITLKSKLVSDFRKDFQFYKVREINNYHHAHDAYLNAVVGTALIKKYPK | | | | |
| | | LESEFVYGDYKVYDVRKMIAKSEQEIGKATAKYFFYSNIMNFFKTEITLANGEI | | | | |
| | | RKRPLIETNGETGEIVWDKGRDFATVRKVLSMPQVNIVKKTEVQTGGFSKES | | | | |
| | | IRPKRNSDKLIARKKDWDPKKYGGFLWPTVAYSVLVVAKVEKGKSKKLKSVK | | | | |
| | | ELLGITIMERSSFEKNPIDFLEAKGYKEVKKDLIIKLPKYSLFELENGRKRMLAS | | | | |
| | | AKQLQKGNELALPSKYVNFLYLASHYEKLKGSPEDNEQKQLFVEQHKHYLDE | | | | |
| | | IIEQISEFSKRVILADANLDKVLSAYNKHRDKPIREQAENIIHLFTLTRLGAPRAF | | | | |
| | | KYFDTTIDPKQYRSTKEVLDATLIHQSITGLYETRIDLSQLGGD | | | | |
| St1Cas9 | Streptococcus | MSDLVLGLDIGIGSVGVGILNKVTGEIIHKNSRIFPAAQAENNLVRRTNRQG | 9,011 | N622A | H599A | D9A |
| | thermophilus | RRLARRKKHRRVRLNRLFEESGLITDFTKISINLNPYQLRVKGLTDELSNEELFI | | | | |
| | | ALKNMVKHRGISYLDDASDDGNSSVGDYAQIVKENSKQLETKTPGQIQLER | | | | |
| | | YQTYGQLRGDFTVEKDGKKHRLINVFPTSAYRSEALRILQTQQEFNPQITDEF | | | | |
| | | INRYLEILTGKRKYYHGPGNEKSRTDYGRYRTSGETLDNIFGILIGKCTFYPDEF | | | | |
| | | RAAKASYTAQEFNLLNDLNNLTVPTETKKLSKEQKNQIINYVKNEKAMGPAK | | | | |
| | | LFKYIAKLLSCDVADIKGYRIDKSGKAEIHTFEAYRKMKTLETLDIEQMDRETL | | | | |
| | | DKLAYVLTLNTEREGIQEALEHEFADGSFSQKQVDELVQFRKANSSIFGKGW | | | | |
| | | HNFSVKLMMELIPELYETSEEQMTILTRLGKQKTTSSSNKTKYIDEKLLTEEIY | | | | |
| | | NPVVAKSVRQAIKIVNAAIKEYGDFDNIVIEMARETNEDDEKKAIQKIQKAN | | | | |
| | | KDEKDAAMLKAANQYNGKAELPHSVFHGHKQLATKIRLWHQQGERCLYT | | | | |
| | | GKTISIHDLINNSNQFEVDHILPLSITFDDSLANKVLVYATANQEKGQRTPYQ | | | | |
| | | ALDSMDDAWSFRELKAFVRESKTLSNKKKEYLLTEEDISKFDVRKKFIERNLV | | | | |
| | | DTRYASRVVLNALQEHFRAHKIDTKVSVVRGQFTSQLRRHWGIEKTRDTYH | | | | |
| | | HHAVDALIIAASSQLNLWKKQKNTLVSYSEDQLLDIETGELISDDEYKESVFK | | | | |
| | | APYQHFVDTLKSKEFEDSILFSYQVDSKFNRKISDATIYATRQAKVGKDKADE | | | | |
| | | TYVLGKIKDIYTQDGYDAFMKIYKKDKSKFLMYRHDPQTFEKVIEPILENYPN | | | | |
| | | KQINEKGKEVPCNPFLKYKEEHGYIRKYSKKGNGPEIKSLKYYDSKLGNHIDIT | | | | |
| | | PKDSNNKVVLQSVSPWRADVYFNKTTGKYEILGLKYADLQFEKGTGTYKISQ | | | | |
| | | EKYNDIKKKEGVDSDSEFKFTLYKNDLLLVKDTETKEQQLFRFLSRTMPKQKH | | | | |
| | | YVELKPYDKQKFEGGEALIKVLGNVANSGQCKKGLGKSNISIYKVRTDVLGN | | | | |
| | | QHIIKNEGDKPKLDF | | | | |
| BlatCas9 | Brevibacillus | MAYTMGIDVGIASCGWAIVDLERQRIIDIGVRTFEKAENPKNGEALAVPRRE | 9,012 | N607A | H584A | D8A |
| | laterosporus | ARSSRRRLRRKKHRIERLKHMFVRNGLAVDIQHLEQTLRSQNEIDVWQLRV | | | | |
| | | DGLDRMLTQKEWLRVLIHLAQRRGFQSNRKTDGSSEDGQVLVNVTENDRL | | | | |
| | | MEEKDYRTVAEMMVKDEKFSDHKRNKNGNYHGVVSRSSLLVEIHTLFETQ | | | | |
| | | RQHHNSLASKDFELEYVNIWSAQRPVATKDQIEKMIGTCTFLPKEKRAPKAS | | | | |
| | | WHFQYFMLLQTINHIRITNVQGTRSLNKEEIEQVVNMALTKSKVSYHDTRKI | | | | |
| | | LDLSEEYQFVGLDYGKEDEKKKVESKETIIKLDDYHKLNKIFNEVELAKGETWE | | | | |
| | | ADDYDTVAYALTFFKDDEDIRDYLQNKYKDSKNRLVKNLANKEYTNELIGKV | | | | |
| | | STLSFRKVGHLSLKALRKIIPFLEQGMTYDKACQAAGFDFQGISKKKRSVVLP | | | | |
| | | VIDQISNPVVNRALTQTRKVINALIKKYGSPETIHIETARELSKTFDERKNITKD | | | | |
| | | YKENRDKNEHAKKHLSELGIINPTGLDIVKYKLWCEQQGRCMYSNQPISFER | | | | |
| | | LKESGYTEVDHIIPYSRSMNDSYNNRVLVMTRENREKGNQTPFEYMGNDT | | | | |
| | | QRWYEFEQRVTTNPQIKKEKRQNLLLKGFTNRRELEMLERNLNDTRYITKYL | | | | |
| | | SHFISTNLEFSPSDKKKKVVNTSGRITSHLRSRWGLEKNRGQNDLHHAMDAI | | | | |
| | | VIAVTSDSFIQQVTNYYKRKERRELNGDDKFPLPWKFFREEVIARLSPNPKEQ | | | | |
| | | IEALPNHFYSEDELADLQPIFVSRMPKRSITGEAHQAQFRRVVGKTKEGKNIT | | | | |
| | | AKKTALVDISYDKNGDFNMYGRETDPATYEAIKERYLEFGGNVKKAFSTDLH | | | | |
| | | KPKKDGTKGPLIKSVRIMENKTLVHPVNKGKGVVYNSSIVRTDVFQRKEKYY | | | | |
| | | LLPVYVTDVTKGKLPNKVIVAKKGYHDWIEVDDSFTFLFSLYPNDLIFIRQNPK | | | | |
| | | KKISLKKRIESHSISDSKEVQEIHAYYKGVDSSTAAIEFIIHDGSYYAKGVGVQN | | | | |
| | | LDCFEKYQVDILGNYFKVKGEKRLELETSDSNHKGKDVNSIKSTSR | | | | |
| cCas9-v16 | Staphylococcus | MKRNYILGLDIGITSVGYGIIDYETRDVIDAGVRLFKEANVENNEGRRSKRGA | 9,013 | N580A | H557A | D10A |
| | aureus | RRLKRRRRHRIQRVKKLLFDYNLLTDHSELSGINPYEARVKGLSQKLSEEEFSA | | | | |
| | | ALLHLAKRRGVHNVNEVEEDTGNELSTKEQISRNSKALEEKYVAELQLERLKK | | | | |
| | | DGEVRGSINRFKTSDYVKEAKQLLKVQKAYHQLDQSFIDTYIDLLETRRTYYE | | | | |
| | | GPGEGSPFGWKDIKEWYEMLMGHCTYFPEELRSVKYAYNADLYNALNDLN | | | | |
| | | NLVITRDENEKLEYYEKFQUIENVFKQKKKPTLKQIAKEILVNEEDIKGYRVTST | | | | |
| | | GKPEFTNLKVYHDIKDITARKEIIENAELLDQIAKILTIYQSSEDIQEELTNLNSE | | | | |
| | | LTQEEIEQISNLKGYTGTHNLSLKAINLILDELWHTNDNQIAIFNRLKLVPKKV | | | | |
| | | DLSQQKEIPTTLVDDFILSPVVKRSFIQSIKVINAIIKKYGLPNDIIIELAREKNSK | | | | |
| | | DAQKMINEMQKRNRQTNERIEEIIRTTGKENAKYLIEKIKLHDMQEGKCLYS | | | | |
| | | LEAIPLEDLLNNPFNYEVDHIIPRSVSFDNSFNNKVLVKQEENSKKGNRTPFQ | | | | |
| | | YLSSSDSKISYETFKKHILNLAKGKGRISKTKKEYLLEERDINRFSVQKDFINRNL | | | | |
| | | VDTRYATRGLMNLLRSYFRVNNLDVKVKSINGGFTSFLRRKWKFKKERNKG | | | | |
| | | YKHHAEDALIIANADFIFKEWKKLDKAKKVMENQMFEEKQAESMPEIETEQ | | | | |
| | | EYKEIFITPHQIKHIKDFKDYKYSHRVDKKPNRKLINDTLYSTRKDDKGNTLIV | | | | |
| | | NNLNGLYDKDNDKLKKLINKSPEKLLMYHHDPQTYQKLKLIMEQYGDEKNP | | | | |
| | | LYKYYEETGNYLTKYSKKDNGPVIKKIKYYGNKLNAHLDITDDYPNSRNKVVK | | | | |
| | | LSLKPYRFDVYLDNGVYKFVTVKNLDVIKKENYYEVNSKCYEEAKKLKKISNQ | | | | |
| | | AEFIASFYKNDLIKINGELYRVIGVNSDKNNLIEVNMIDITYREYLENMNDKRP | | | | |
| | | PHIIKTIASKTQSIKKYSTDILGNLYEVKSKKHPQIIKKG | | | | |
| cCas9-v17 | Staphylococcus | MKRNYILGLDIGITSVGYGIIDYETRDVIDAGVRLFKEANVENNEGRRSKRGA | 9,014 | N580A | H557A | D10A |
| | aureus | RRLKRRRRHRIQRVKKLLFDYNLLTDHSELSGINPYEARVKGLSQKLSEEEFSA | | | | |
| | | ALLHLAKRRGVHNVNEVEEDTGNELSTKEQISRNSKALEEKYVAELQLERLKK | | | | |
| | | DGEVRGSINRFKTSDYVKEAKQLLKVQKAYHQLDQSFIDTYIDLLETRRTYYE | | | | |
| | | GPGEGSPFGWKDIKEWYEMLMGHCTYFPEELRSVKYAYNADLYNALNDLN | | | | |
| | | NLVITRDENEKLEYYEKFQIIENVFKQKKKPTLKQIAKEILVNEEDIKGYRVTST | | | | |
| | | GKPEFTNLKVYHDIKDITARKEIIENAELLDQIAKILTIYQSSEDIQEELTNLNSE | | | | |
| | | LTQEEIEQISNLKGYTGTHNLSLKAINLILDELWHTNDNQIAIFNRLKLVPKKV | | | | |
| | | DLSQQKEIPTTLVDDFILSPVVKRSFIQSIKVINAIIKKYGLPNDIIIELAREKNSK | | | | |
| | | DAQKMINEMQKRNRQTNERIEEIIRTTGKENAKYLIEKIKLHDMQEGKCLYS | | | | |
| | | LEAIPLEDLLNNPFNYEVDHIIPRSVSFDNSFNNKVLVKQEENSKKGNRTPFQ | | | | |
| | | YLSSSDSKISYETFKKHILNLAKGKGRISKTKKEYLLEERDINRFSVQKDFINRNL | | | | |
| | | VDTRYATRGLMNLLRSYFRVNNLDVKVKSINGGFTSFLRRKWKFKKERNKG | | | | |
| | | YKHHAEDALIIANADFIFKEWKKLDKAKKVMENQMFEEKQAESMPEIETEQ | | | | |
| | | EYKEIFITPHQIKHIKDFKDYKYSHRVDKKPNRKLINDTLYSTRKDDKGNTLIV | | | | |
| | | NNLNGLYDKDNDKLKKLINKSPEKLLMYHHDPQTYQKLKLIMEQYGDEKNP | | | | |
| | | LYKYYEETGNYLTKYSKKDNGPVIKKIKYYGNKLNAHLDITDDYPNSRNKVVK | | | | |
| | | LSLKPYRFDVYLDNGVYKFVTVKNLDVIKKENYYEVNSKCYEEAKKLKKISNQ | | | | |
| | | AEFIASFYKNDLIKINGELYRVIGVNNSTRNIVELNMIDITYREYLENMNDKRP | | | | |
| | | PHIIKTIASKTQSIKKYSTDILGNLYEVKSKKHPQIIKKG | | | | |
| cCas9-v21 | Staphylococcus | MKRNYILGLDIGITSVGYGIIDYETRDVIDAGVRLFKEANVENNEGRRSKRGA | 9,015 | N580A | H557A | D10A |
| | aureus | RRLKRRRRHRIQRVKKLLFDYNLLTDHSELSGINPYEARVKGLSQKLSEEEFSA | | | | |
| | | ALLHLAKRRGVHNVNEVEEDTGNELSTKEQISRNSKALEEKYVAELQLERLKK | | | | |
| | | DGEVRGSINRFKTSDYVKEAKQLLKVQKAYHQLDQSFIDTYIDLLETRRTYYE | | | | |
| | | GPGEGSPFGWKDIKEWYEMLMGHCTYFPEELRSVKYAYNADLYNALNDLN | | | | |
| | | NLVITRDENEKLEYYEKFQIIENVFKQKKKPTLKQIAKEILVNEEDIKGYRVTST | | | | |
| | | GKPEFTNLKVYHDIKDITARKEIIENAELLDQIAKILTIYQSSEDIQEELTNLNSE | | | | |
| | | LTQEEIEQISNLKGYTGTHNLSLKAINLILDELWHTNDNQIAIFNRLKLVPKKV | | | | |
| | | DLSQQKEIPTTLVDDFILSPVVKRSFIQSIKVINAIIKKYGLPNDIIIELAREKNSK | | | | |
| | | DAQKMINEMQKRNRQTNERIEEIIRTTGKENAKYLIEKIKLHDMQEGKCLYS | | | | |
| | | LEAIPLEDLLNNPFNYEVDHIIPRSVSFDNSFNNKVLVKQEENSKKGNRTPFQ | | | | |
| | | YLSSSDSKISYETFKKHILNLAKGKGRISKTKKEYLLEERDINRFSVQKDFINRNL | | | | |
| | | VDTRYATRGLMNLLRSYFRVNNLDVKVKSINGGFTSFLRRKWKFKKERNKG | | | | |
| | | YKHHAEDALIANADFIFKEWKKLDKAKKVMENQMFEEKQAESMPEIETEQ | | | | |
| | | EYKEIFITPHQIKHIKDFKDYKYSHRVDKKPNRKLINDTLYSTRKDDKGNTLIV | | | | |
| | | NNLNGLYDKDNDKLKKLINKSPEKLLMYHHDPQTYQKLKLIMEQYGDEKNP | | | | |
| | | LYKYYEETGNYLTKYSKKDNGPVIKKIKYYGNKLNAHLDITDDYPNSRNKVVK | | | | |
| | | LSLKPYRFDVYLDNGVYKFVTVKNLDVIKKENYYEVNSKCYEEAKKLKKISNQ | | | | |
| | | AEFIASFYKNDLIKINGELYRVIGVNSDDRNIIELNMIDITYREYLENMNDKRP | | | | |
| | | PHIIKTIASKTQSIKKYSTDILGNLYEVKSKKHPQIIKKG | | | | |
| cCas9-v42 | | MKRNYILGLDIGITSVGYGIIDYETRDVIDAGVRLFKEANVENNEGRRSKRGA | 9,016 | N580A | H557A | D10A |
| | Staphylococcus | RRLKRRRRHRIQRVKKLLFDYNLLTDHSELSGINPYEARVKGLSQKLSEEEFSA | | | | |
| | aureus | ALLHLAKRRGVHNVNEVEEDTGNELSTKEQISRNSKALEEKYVAELQLERLKK | | | | |
| | | DGEVRGSINRFKTSDYVKEAKQLLKVQKAYHQLDQSFIDTYIDLLETRRTYYE | | | | |
| | | GPGEGSPFGWKDIKEWYEMLMGHCTYFPEELRSVKYAYNADLYNALNDLN | | | | |
| | | NLVITRDENEKLEYYEKFQIIENVFKQKKKPTLKQIAKEILVNEEDIKGYRVTST | | | | |
| | | GKPEFTNLKVYHDIKDITARKEIIENAELLDQIAKILTIYQSSEDIQEELTNLNSE | | | | |
| | | LTQEEIEQISNLKGYTGTHNLSLKAINLILDELWHTNDNQIAIFNRLKLVPKKV | | | | |
| | | DLSQQKEIPTTLVDDFILSPVVKRSFIQSIKVINAIIKKYGLPNDIIIELAREKNSK | | | | |
| | | DAQKMINEMQKRNRQTNERIEEIIRTTGKENAKYLIEKIKLHDMQEGKCLYS | | | | |
| | | LEAIPLEDLLNNPFNYEVDHIIPRSVSFDNSFNNKVLVKQEENSKKGNRTPFQ | | | | |
| | | YLSSSDSKISYETFKKHILNLAKGKGRISKTKKEYLLEERDINRFSVQKDFINRNL | | | | |
| | | VDTRYATRGLMNLLRSYFRVNNLDVKVKSINGGFTSFLRRKWKFKKERNKG | | | | |
| | | YKHHAEDALIIANADFIFKEWKKLDKAKKVMENQMFEEKQAESMPEIETEQ | | | | |
| | | EYKEIFITPHQIKHIKDFKDYKYSHRVDKKPNRKLINDTLYSTRKDDKGNTLIV | | | | |
| | | NNLNGLYDKDNDKLKKLINKSPEKLLMYHHDPQTYQKLKLIMEQYGDEKNP | | | | |
| | | LYKYYEETGNYLTKYSKKDNGPVIKKIKYYGNKLNAHLDITDDYPNSRNKVVK | | | | |
| | | LSLKPYRFDVYLDNGVYKFVTVKNLDVIKKENYYEVNSKCYEEAKKLKKISNQ | | | | |
| | | AEFIASFYKNDLIKINGELYRVIGVNNNRLNKIELNMIDITYREYLENMNDKRP | | | | |
| | | PHIIKTIASKTQSIKKYSTDILGNLYEVKSKKHPQIIKKG | | | | |
| CdiCas9 | Corynebacterium | MKYHVGIDVGTFSVGLAAIEVDDAGMPIKTLSLVSHIHDSGLDPDEIKSAVT | 9,017 | N597A | H573A | D8A |
| | diphtheriae | RLASSGIARRTRRLYRRKRRRLQQLDKFIQRQGWPVIELEDYSDPLYPWKVR | | | | |
| | | AELAASYIADEKERGEKLSVALRHIARHRGWRNPYAKVSSLYLPDGPSDAFK | | | | |
| | | AIREEIKRASGQPVPETATVGQMVTLCELGTLKLRGEGGVLSARLQQSDYAR | | | | |
| | | EIQEICRMQEIGQELYRKIIDVVFAAESPKGSASSRVGKDPLQPGKNRALKAS | | | | |
| | | DAFQRYRIAALIGNLRVRVDGEKRILSVEEKNLVFDHLVNLTPKKEPEWVTIA | | | | |
| | | EILGIDRGQLIGTATMTDDGERAGARPPTHDTNRSIVNSRIAPLVDWWKTA | | | | |
| | | SALEQHAMVKALSNAEVDDFDSPEGAKVQAFFADLDDDVHAKLDSLHLPV | | | | |
| | | GRAAYSEDTLVRLTRRMLSDGVDLYTARLQEFGIEPSWTPPTPRIGEPVGNP | | | | |
| | | AVDRVLKTVSRWLESATKTWGAPERVIIEHVREGFVTEKRAREMDGDMRR | | | | |
| | | RAARNAKLFQEMQEKLNVQGKPSRADLWRYQSVQRQNCQCAYCGSPITF | | | | |
| | | SNSEMDHIVPRAGQGSTNTRENLVAVCHRCNQSKGNTPFAIWAKNTSIEG | | | | |
| | | VSVKEAVERTRHWVTDTGMRSTDFKKFTKAVVERFQRATMDEEIDARSME | | | | |
| | | SVAWMANELRSRVAQHFASHGTTVRVYRGSLTAEARRASGISGKLKFFDGV | | | | |
| | | GKSRLDRRHHAIDAAVIAFTSDYVAETLAVRSNLKQSQAHRQEAPQWREFT | | | | |
| | | GKDAEHRAAWRVWCQKMEKLSALLTEDLRDDRVVVMSNVRLRLGNGSA | | | | |
| | | HKETIGKLSKVKLSSQLSVSDIDKASSEALWCALTREPGFDPKEGLPANPERHI | | | | |
| | | RVNGTHVYAGDNIGLFPVSAGSIALRGGYAELGSSFHHARVYKITSGKKPAF | | | | |
| | | AMLRVYTIDLLPYRNQDLFSVELKPQTMSMRQAEKKLRDALATGNAEYLG | | | | |
| | | WLVVDDELVVDTSKIATDQVKAVEAELGTIRRWRVDGFFSPSKLRLRPLQM | | | | |
| | | SKEGIKKESAPELSKIIDRPGWLPAVNKLFSDGNVTVVRRDSLGRVRLESTAH | | | | |
| | | LPVTWKVQ | | | | |
| CjeCas9 | Campylobacter | MARILAFDIGISSIGWAFSENDELKDCGVRIFTKVENPKTGESLALPRRLARSA | 9,018 | N582A | H559A | D8A |
| | jejuni | RKRLARRKARLNHLKHLIANEFKLNYEDYQSFDESLAKAYKGSLISPYELRFRA | | | | |
| | | LNELLSKQDFARVILHIAKRRGYDDIKNSDDKEKGAILKAIKQNEEKLANYQS | | | | |
| | | VGEYLYKEYFQKFKENSKEFTNVRNKKESYERCIAQSFLKDELKLIFKKQREFG | | | | |
| | | FSFSKKFEEEVLSVAFYKRALKDFSHLVGNCSFFTDEKRAPKNSPLAFMFVAL | | | | |
| | | TRIINLLNNLKNTEGILYTKDDLNALLNEVLKNGTLTYKQTKKLLGLSDDYEFK | | | | |
| | | GEKGTYFIEFKKYKEFIKALGEHNLSQDDLNEIAKDITLIKDEIKLKKALAKYDLN | | | | |
| | | QNQIDSLSKLEFKDHLNISFKALKLVTPLMLEGKKYDEACNELNLKVAINEDK | | | | |
| | | KDFLPAFNETYYKDEVTNPVVLRAIKEYRKVLNALLKKYGKVHKINIELAREVG | | | | |
| | | KNHSQRAKIEKEQNENYKAKKDAELECEKLGLKINSKNILKLRLFKEQKEFCAY | | | | |
| | | SGEKIKISDLQDEKMLEIDHIYPYSRSFDDSYMNKVLVFTKQNQEKLNQTPFE | | | | |
| | | AFGNDSAKWQKIEVLAKNLPTKKQKRILDKNYKDKEQKNFKDRNLNDTRYI | | | | |
| | | ARLVLNYTKDYLDFLPLSDDENTKLNDTQKGSKVHVEAKSGMLTSALRHTW | | | | |
| | | GFSAKDRNNHLHHAIDAVIIAYANNSIVKAFSDFKKEQESNSAELYAKKISELD | | | | |
| | | YKNKRKFFEPFSGFRQKVLDKIDEIFVSKPERKKPSGALHEETFRKEEEFYQSY | | | | |
| | | GGKEGVLKALELGKIRKVNGKIVKNGDMFRVDIFKHKKTNKFYAVPIYTMDF | | | | |
| | | ALKVLPNKAVARSKKGEIKDWILMDENYEFCFSLYKDSLILIQTKDMQEPEFV | | | | |
| | | YYNAFTSSTVSLIVSKHDNKFETLSKNQKILFKNANEKEVIAKSIGIQNLKVFEK | | | | |
| | | YIVSALGEVTKAEFRQREDFKK | | | | |
| GeoCas9 | Geobacillus | MRYKIGLDIGITSVGWAVMNLDIPRIEDLGVRIFDRAENPQTGESLALPRRLA | 9,019 | N605A | H582A | D8A |
| | stearothermophilus | RSARRRLRRRKHRLERIRRLVIREGILTKEELDKLFEEKHEIDVWQLRVEALDR | | | | |
| | | KLNNDELARVLLHLAKRRGFKSNRKSERSNKENSTMLKHIEENRAILSSYRTV | | | | |
| | | GEMIVKDPKFALHKRNKGENYTNTIARDDLEREIRLIFSKQREFGNMSCTEEF | | | | |
| | | ENEYITIWASQRPVASKDDIEKKVGFCTFEPKEKRAPKATYTFQSFIAWEHIN | | | | |
| | | KLRLISPSGARGLTDEERRLLYEQAFQKNKITYHDIRTLLHLPDDTYFKGIVYDR | | | | |
| | | GESRKQNENIRFLELDAYHQIRKAVDKVYGKGKSSSFLPIDFDTFGYALTLFKD | | | | |
| | | DADIHSYLRNEYEQNGKRMPNLANKVYDNELIEELLNLSFTKFGHLSLKALRS | | | | |
| | | ILPYMEQGEVYSSACERAGYTFTGPKKKQKTMLLPNIPPIANPVVMRALTQA | | | | |
| | | RKVVNAIIKKYGSPVSIHIELARDLSQTFDERRKTKKEQDENRKKNETAIRQL | | | | |
| | | MEYGLTLNPTGHDIVKFKLWSEQNGRCAYSLQPIEIERLLEPGYVEVDHVIPY | | | | |
| | | SRSLDDSYTNKVLVLTRENREKGNRIPAEYLGVGTERWQQFETFVLTNKQFS | | | | |
| | | KKKRDRLLRLHYDENEETEFKNRNLNDTRYISRFFANFIREHLKFAESDDKQK | | | | |
| | | VYTVNGRVTAHLRSRWEFNKNREESDLHHAVDAVIVACTTPSDIAKVTAFY | | | | |
| | | QRREQNKELAKKTEPHFPQPWPHFADELRARLSKHPKESIKALNLGNYDDQ | | | | |
| | | KLESLQPVFVSRMPKRSVTGAAHQETLRRYVGIDERSGKIQTVVKTKLSEIKL | | | | |
| | | DASGHFPMYGKESDPRTYEAIRQRLLEHNNDPKKAFQEPLYKPKKNGEPGP | | | | |
| | | VIRTVKIIDTKNQVIPLNDGKTVAYNSNIVRVDVFEKDGKYYCVPVYTMDIM | | | | |
| | | KGILPNKAIEPNKPYSEWKEMTEDYTFRFSLYPNDLIRIELPREKTVKTAAGEE | | | | |
| | | INVKDVFVYYKTIDSANGGLELISHDHRFSLRGVGSRTLKRFEKYQVDVLGNI | | | | |
| | | YKVRGEKRVGLASSAHSKPGKTIRPLQSTRD | | | | |
| iSpyMacCas9 | Streptococcus | MDKKYSIGLDIGTNSVGWAVITDEYKVPSKKFKVLGNTDRHSIKKNLIGALLF | 9,020 | N863A | H840A | D10A |
| | spp. | DSGETAEATRLKRTARRRYTRRKNRICYLQEIFSNEMAKVDDSFFHRLEESFL | | | | |
| | | VEEDKKHERHPIFGNIVDEVAYHEKYPTIYHLRKKLVDSTDKADLRLIYLALAH | | | | |
| | | MIKFRGHFLIEGDLNPDNSDVDKLFIQLVQTYNQLFEENPINASGVDAKAILS | | | | |
| | | ARLSKSRKLENLIAQLPGEKKNGLFGNLIALSLGLTPNFKSNFDLAEDAKLQLS | | | | |
| | | KDTYDDDLDNLLAQIGDQYADLFLAAKNLSDAILLSDILRVNTEITKAPLSAS | | | | |
| | | MIKRYDEHHQDLTLLKALVRQQLPEKYKEIFFDQSKNGYAGYIDGGASQEEF | | | | |
| | | YKFIKPILEKMDGTEELLVKLKREDLLRKQRTFDNGSIPHQIHLGELHAILRRQ | | | | |
| | | EDFYPFLKDNREKIEKILTFRIPYYVGPLARGNSRFAWMTRKSEETITPWNFE | | | | |
| | | EVVDKGASAQSFIERMTNFDKNLPNEKVLPKHSLLYEYFTVYNELTKVKYVTE | | | | |
| | | GMRKPAFLSGEQKKAIVDLLFKTNRKVTVKQLKEDYFKKIECFDSVEISGVED | | | | |
| | | RFNASLGTYHDLLKIIKDKDFLDNEENEDILEDIVLTLTLFEDREMIEERLKTYA | | | | |
| | | HLFDDKVMKQLKRRRYTGWGRLSRKLINGIRDKQSGKTILDFLKSDGFANR | | | | |
| | | NFMQLIHDDSLTFKEDIQKAQVSGQGDSLHEHIANLAGSPAIKKGILQTVKV | | | | |
| | | VDELVKVMGRHKPENIVIEMARENQTTQKGQKNSRERMKRIEEGIKELGSQ | | | | |
| | | ILKEHPVENTQLQNEKLYLYYLQNGRDMYVDQELDINRLSDYDVDHIVPQSF | | | | |
| | | LKDDSIDNKVLTRSDKNRGKSDNVPSEEVVKKMKNYWRQLLNAKLITQRKF | | | | |
| | | DNLTKAERGGLSELDKAGFIKRQLVETRQITKHVAQILDSRMNTKYDENDKLI | | | | |
| | | REVKVITLKSKLVSDFRKDFQFYKVREINNYHHAHDAYLNAVVGTALIKKYPK | | | | |
| | | LESEFVYGDYKVYDVRKMIAKSEQEIGKATAKYFFYSNIMNFFKTEITLANGEI | | | | |
| | | RKRPLIETNGETGEIVWDKGRDFATVRKVLSMPQVNIVKKTEIQTVGQNGG | | | | |
| | | LFDDNPKSPLEVTPSKLVPLKKELNPKKYGGYQKPTTAYPVLLITDTKQLIPISV | | | | |
| | | MNKKQFEQNPVKFLRDRGYQQVGKNDFIKLPKYTLVDIGDGIKRLWASSKEI | | | | |
| | | HKGNQLVVSKKSQILLYHAHHLDSDLSNDYLQNHNQQFDVLFNEIISFSKKC | | | | |
| | | KLGKEHIQKIENVYSNKKNSASIEELAESFIKLLGFTQLGATSPFNFLGVKLNQ | | | | |
| | | KQYKGKKDYILPCTEGTLIRQSITGLYETRVDLSKIGEDSGGSGGSKRTADGSE | | | | |
| | | FES | | | | |
| NmeCas9 | Neisseria | MAAFKPNSINYILGLDIGIASVGWAMVEIDEEENPIRLIDLGVRVFERAEVPK | 9,021 | N611A | H588A | D16A |
| | meningitidis | TGDSLAMARRLARSVRRLTRRRAHRLLRTRRLLKREGVLQAANFDENGLIKS | | | | |
| | | LPNTPWQLRAAALDRKLTPLEWSAVLLHLIKHRGYLSQRKNEGETADKELG | | | | |
| | | ALLKGVAGNAHALQTGDFRTPAELALNKFEKESGHIRNQRSDYSHTFSRKDL | | | | |
| | | QAELILLFEKQKEFGNPHVSGGLKEGIETLLMTQRPALSGDAVQKMLGHCTF | | | | |
| | | EPAEPKAAKNTYTAERFIWLTKLNNLRILEQGSERPLTDTERATLMDEPYRKS | | | | |
| | | KLTYAQARKLLGLEDTAFFKGLRYGKDNAEASTLMEMKAYHAISRALEKEGL | | | | |
| | | KDKKSPLNLSPELQDEIGTAFSLFKTDEDITGRLKDRIQPEILEALLKHISFDKFV | | | | |
| | | QISLKALRRIVPLMEQGKRYDEACAEIYGDHYGKKNTEEKIYLPPIPADEIRNP | | | | |
| | | VVLRALSQARKVINGVVRRYGSPARIHIETAREVGKSFKDRKEIEKRQEENRK | | | | |
| | | DREKAAAKFREYFPNFVGEPKSKDILKLRLYEQQHGKCLYSGKEINLGRLNEK | | | | |
| | | GYVEIDHALPFSRTWDDSFNNKVLVLGSENQNKGNQTPYEYFNGKDNSRE | | | | |
| | | WQEFKARVETSRFPRSKKQRILLQKFDEDGFKERNLNDTRYVNRFLCQFVA | | | | |
| | | DRMRLTGKGKKRVFASNGQITNLLRGFWGLRKVRAENDRHHALDAVVVA | | | | |
| | | CSTVAMQQKITRFVRYKEMNAFDGKTIDKETGEVLHQKTHFPQPWEFFAQ | | | | |
| | | EVMIRVFGKPDGKPEFEEADTLEKLRTLLAEKLSSRPEAVHEYVTPLFVSRAP | | | | |
| | | NRKMSGQGHMETVKSAKRLDEGVSVLRVPLTQLKLKDLEKMVNREREPKL | | | | |
| | | YEALKARLEAHKDDPAKAFAEPFYKYDKAGNRTQQVKAVRVEQVQKTGVW | | | | |
| | | VRNHNGIADNATMVRVDVFEKGDKYYLVPIYSWQVAKGILPDRAVVQGKD | | | | |
| | | EEDWQLIDDSFNFKFSLHPNDLVEVITKKARMFGYFASCHRGTGNINIRIHD | | | | |
| | | LDHKIGKNGILEGIGVKTALSFQKYQIDELGKEIRPCRLKKRPPVR | | | | |
| ScaCas9 | Streptococcus | MEKKYSIGLDIGTNSVGWAVITDDYKVPSKKFKVLGNTNRKSIKKNLMGALL | 9,022 | N872A | H849A | D10A |
| | canis | FDSGETAEATRLKRTARRRYTRRKNRIRYLQEIFANEMAKLDDSFFQRLEESF | | | | |
| | | LVEEDKKNERHPIFGNLADEVAYHRNYPTIYHLRKKLADSPEKADLRLIYLALA | | | | |
| | | HIIKFRGHFLIEGKLNAENSDVAKLFYQLIQTYNQLFEESPLDEIEVDAKGILSA | | | | |
| | | RLSKSKRLEKLIAVFPNEKKNGLFGNIIALALGLTPNFKSNFDLTEDAKLQLSKD | | | | |
| | | TYDDDLDELLGQIGDQYADLFSAAKNLSDAILLSDILRSNSEVTKAPLSASMV | | | | |
| | | KRYDEHHQDLALLKTLVRQQFPEKYAEIFKDDTKNGYAGYVGIGIKHRKRTT | | | | |
| | | KLATQEEFYKFIKPILEKMDGAEELLAKLNRDDLLRKQRTFDNGSIPHQIHLKE | | | | |
| | | LHAILRRQEEFYPFLKENREKIEKILTFRIPYYVGPLARGNSRFAWLTRKSEEAI | | | | |
| | | TPWNFEEVVDKGASAQSFIERMTNFDEQLPNKKVLPKHSLLYEYFTVYNELT | | | | |
| | | KVKYVTERMRKPEFLSGEQKKAIVDLLFKTNRKVTVKQLKEDYFKKIECFDSV | | | | |
| | | EIIGVEDRFNASLGTYHDLLKIIKDKDFLDNEENEDILEDIVLTLTLFEDREMIEE | | | | |
| | | RLKTYAHLFDDKVMKQLKRRHYTGWGRLSRKMINGIRDKQSGKTILDFLKS | | | | |
| | | DGFSNRNFMQLIHDDSLTFKEEIEKAQVSGQGDSLHEQIADLAGSPAIKKGIL | | | | |
| | | QTVKIVDELVKVMGHKPENIVIEMARENQTTTKGLQQSRERKKRIEEGIKELE | | | | |
| | | SQILKENPVENTQLQNEKLYLYYLQNGRDMYVDQELDINRLSDYDVDHIVP | | | | |
| | | QSFIKDDSIDNKVLTRSVENRGKSDNVPSEEVVKKMKNYWRQLLNAKLITQ | | | | |
| | | RKFDNLTKAERGGLSEADKAGFIKRQLVETRQITKHVARILDSRMNTKRDKN | | | | |
| | | DKPIREVKVITLKSKLVSDFRKDFQLYKVRDINNYHHAHDAYLNAVVGTALIK | | | | |
| | | KYPKLESEFVYGDYKVYDVRKMIAKSEQEIGKATAKRFFYSNIMNFFKTEVKL | | | | |
| | | ANGEIRKRPLIETNGETGEVVWNKEKDFATVRKVLAMPQVNIVKKTEVQTG | | | | |
| | | GFSKESILSKRESAKLIPRKKGWDTRKYGGFGSPTVAYSILVVAKVEKGKAKKL | | | | |
| | | KSVKVLVGITIMEKGSYEKDPIGFLEAKGYKDIKKELIFKLPKYSLFELENGRRR | | | | |
| | | MLASATELQKANELVLPQHLVRLLYYTQNISATTGSNNLGYIEQHREEFKEIF | | | | |
| | | EKIIDFSEKYILKNKVNSNLKSSFDEQFAVSDSILLSNSFVSLLKYTSFGASGGFT | | | | |
| | | FLDLDVKQGRLRYQTVTEVLDATLIYQSITGLYETRTDLSQLGGD | | | | |
| ScaCas9- | Streptococcus | MEKKYSIGLDIGTNSVGWAVITDDYKVPSKKFKVLGNTNRKSIKKNLMGALL | 9,023 | N872A | H849A | D10A |
| HiFi-Sc++ | canis | FDSGETAEATRLKRTARRRYTRRKNRIRYLQEIFANEMAKLDDSFFQRLEESF | | | | |
| | | LVEEDKKNERHPIFGNLADEVAYHRNYPTIYHLRKKLADSPEKADLRLIYLALA | | | | |
| | | HIIKFRGHFLIEGKLNAENSDVAKLFYQLIQTYNQLFEESPLDEIEVDAKGILSA | | | | |
| | | RLSKSKRLEKLIAVFPNEKKNGLFGNIIALALGLTPNFKSNFDLTEDAKLQLSKD | | | | |
| | | TYDDDLDELLGQIGDQYADLFSAAKNLSDAILLSDILRSNSEVTKAPLSASMV | | | | |
| | | KRYDEHHQDLALLKTLVRQQFPEKYAEIFKDDTKNGYAGYVGADKKLRKRS | | | | |
| | | GKLATEEEFYKFIKPILEKMDGAEELLAKLNRDDLLRKQRTFDNGSIPHQIHLK | | | | |
| | | ELHAILRRQEEFYPFLKENREKIEKILTFRIPYYVGPLARGNSRFAWLTRKSEEA | | | | |
| | | ITPWNFEEVVDKGASAQSFIERMTNFDEQLPNKKVLPKHSLLYEYFTVYNEL | | | | |
| | | TKVKYVTERMRKPEFLSGEQKKAIVDLLFKTNRKVTVKQLKEDYFKKIECFDS | | | | |
| | | VEIIGVEDRFNASLGTYHDLLKIIKDKDFLDNEENEDILEDIVLTLTLFEDREMIE | | | | |
| | | ERLKTYAHLFDDKVMKQLKRRHYTGWGRLSRKMINGIRDKQSGKTILDFLKS | | | | |
| | | DGFSNANFMQLIHDDSLTFKEEIEKAQVSGQGDSLHEQIADLAGSPAIKKGIL | | | | |
| | | QTVKIVDELVKVMGHKPENIVIEMARENQTTTKGLQQSRERKKRIEEGIKELE | | | | |
| | | SQILKENPVENTQLQNEKLYLYYLQNGRDMYVDQELDINRLSDYDVDHIVP | | | | |
| | | QSFIKDDSIDNKVLTRSVENRGKSDNVPSEEVVKKMKNYWRQLLNAKLITQ | | | | |
| | | RKFDNLTKAERGGLSEADKAGFIKRQLVETRQITKHVARILDSRMNTKRDKN | | | | |
| | | DKPIREVKVITLKSKLVSDFRKDFQLYKVRDINNYHHAHDAYLNAVVGTALIK | | | | |
| | | KYPKLESEFVYGDYKVYDVRKMIAKSEQEIGKATAKRFFYSNIMNFFKTEVKL | | | | |
| | | ANGEIRKRPLIETNGETGEVVWNKEKDFATVRKVLAMPQVNIVKKTEVQTG | | | | |
| | | GFSKESILSKRESAKLIPRKKGWDTRKYGGFGSPTVAYSILVVAKVEKGKAKKL | | | | |
| | | KSVKVLVGITIMEKGSYEKDPIGFLEAKGYKDIKKELIFKLPKYSLFELENGRRR | | | | |
| | | MLASAKELQKANELVLPQHLVRLLYYTQNISATTGSNNLGYIEQHREEFKEIF | | | | |
| | | EKIIDFSEKYILKNKVNSNLKSSFDEQFAVSDSILLSNSFVSLLKYTSFGASGGFT | | | | |
| | | FLDLDVKQGRLRYQTVTEVLDATLIYQSITGLYETRTDLSQLGGD | | | | |
| SpyCas9- | Streptococcus | MDKKYSIGLDIGTNSVGWAVITDEYKVPSKKFKVLGNTDRHSIKKNLIGALLF | 9,024 | N863A | H840A | D10A |
| 3var-NRRH | pyogenes | DSGETAEATRLKRTARRRYTRRKNRICYLQEIFSNEMAKVDDSFFHRLEESFL | | | | |
| | | VEEDKKHERHPIFGNIVDEVAYHEKYPTIYHLRKKLVDSTDKADLRLIYLALAH | | | | |
| | | MIKFRGHFLIEGDLNPDNSDVDKLFIQLVQTYNQLFEENPINASGVDAKAILS | | | | |
| | | ARLSKSRRLENLIAQLPGEKKNGLFGNLIALSLGLTPNFKSNFDLAEDAKLQLS | | | | |
| | | KDTYDDDLDNLLAQIGDQYADLFLAAKNLSDAILLSDILRVNTEITKAPLSAS | | | | |
| | | MVKRYDEHHQDLTLLKALVRQQLPEKYKEIFFDQSKNGYAGYIDGGASQEE | | | | |
| | | FYKFIKPILEKMDGTEELLVKLNREDLLRKQRTFDNGIIPHQIHLGELHAILRRQ | | | | |
| | | GDFYPFLKDNREKIEKILTFRIPYYVGPLARGNSRFAWMTRKSEETITPWNFE | | | | |
| | | EVVDKGASAQSFIERMTNFDKNLPNEKVLPKHSLLYEYFTVYNELTKVKYVTE | | | | |
| | | GMRKPAFLSGEQKKAIVDLLFKTNRKVTVKQLKEDYFKKIECFDSVEISGVED | | | | |
| | | RFNASLGTYHDLLKIIKDKDFLDNEENEDILEDIVLTLTLFEDREMIEERLKTYA | | | | |
| | | HLFDDKVMKQLKRLRYTGWGRLSRKLINGIRDKQSGKTILDFLKSDGFANRN | | | | |
| | | FMQLIHDDSLTFKEDIQKAQVSGQGDSLHEHIANLAGSPAIKKGILQTVKVV | | | | |
| | | DELVKVMGGHKPENIVIEMARENQTTQKGQKNSRERMKRIEEGIKELGSQI | | | | |
| | | LKEHPVENTQLQNEKLYLYYLQNGRDMYVDQELDINRLSDYDVDHIVPQSF | | | | |
| | | LKDDSIDNKVLTRSDKNRGKSDNVPSEEVVKKMKNYWRQLLNAKLITQRKF | | | | |
| | | DNLTKAERGGLSELDKAGFIKRQLVETRQITKHVAQILDSRMNTKYDENDKLI | | | | |
| | | REVKVITLKSKLVSDFRKDFQFYKVREINNYHHAHDAYLNAVVGTALIKKYPK | | | | |
| | | LESEFVYGDYKVYDVRKMIAKSEQEIGKATAKYFFYSNIMNFFKTEITLANGEI | | | | |
| | | RKRPLIETNGETGEIVWDKGRDFATVRKVLSMPQVNIVKKTEVQTGGFSKES | | | | |
| | | ILPKGNSDKLIARKKDWDPKKYGGFNSPTAAYSVLVVAKVEKGKSKKLKSVK | | | | |
| | | ELLGITIMERSSFEKNPIGFLEAKGYKEVKKDLIIKLPKYSLFELENGRKRMLAS | | | | |
| | | AGVLHKGNELALPSKYVNFLYLASHYEKLKGSPEDNEQKQLFVEQHKHYLDE | | | | |
| | | IIEQISEFSKRVILADANLDKVLSAYNKHRDKPIREQAENIIHLFTLTNLGVPAA | | | | |
| | | FKYFDTTIDKKRYTSTKEVLDATLIHQSITGLYETRIDLSQLGGD | | | | |
| SpyCas9- | Streptococcus | MDKKYSIGLDIGTNSVGWAVITDEYKVPSKKFKVLGNTDRHSIKKNLIGALLF | 9,025 | N863A | H840A | D10A |
| 3var-NRTH | pyogenes | DSGETAEATRLKRTARRRYTRRKNRICYLQEIFSNEMAKVDDSFFHRLEESFL | | | | |
| | | VEEDKKHERHPIFGNIVDEVAYHEKYPTIYHLRKKLVDSTDKADLRLIYLALAH | | | | |
| | | MIKFRGHFLIEGDLNPDNSDVDKLFIQLVQTYNQLFEENPINASGVDAKAILS | | | | |
| | | ARLSKSRRLENLIAQLPGEKKNGLFGNLIALSLGLTPNFKSNFDLAEDAKLQLS | | | | |
| | | KDTYDDDLDNLLAQIGDQYADLFLAAKNLSDAILLSDILRVNTEITKAPLSAS | | | | |
| | | MVKRYDEHHQDLTLLKALVRQQLPEKYKEIFFDQSKNGYAGYIDGGASQEE | | | | |
| | | FYKFIKPILEKMDGTEELLVKLNREDLLRKQRTFDNGIIPHQIHLGELHAILRRQ | | | | |
| | | GDFYPFLKDNREKIEKILTFRIPYYVGPLARGNSRFAWMTRKSEETITPWNFE | | | | |
| | | EVVDKGASAQSFIERMTNFDKNLPNEKVLPKHSLLYEYFTVYNELTKVKYVTE | | | | |
| | | GMRKPAFLSGEQKKAIVDLLFKTNRKVTVKQLKEDYFKKIECFDSVEISGVED | | | | |
| | | RFNASLGTYHDLLKIIKDKDFLDNEENEDILEDIVLTLTLFEDREMIEERLKTYA | | | | |
| | | HLFDDKVMKQLKRLRYTGWGRLSRKLINGIRDKQSGKTILDFLKSDGFANRN | | | | |
| | | FMQLIHDDSLTFKEDIQKAQVSGQGDSLHEHIANLAGSPAIKKGILQTVKVV | | | | |
| | | DELVKVMGGHKPENIVIEMARENQTTQKGQKNSRERMKRIEEGIKELGSQI | | | | |
| | | LKEHPVENTQLQNEKLYLYYLQNGRDMYVDQELDINRLSDYDVDHIVPQSF | | | | |
| | | LKDDSIDNKVLTRSDKNRGKSDNVPSEEVVKKMKNYWRQLLNAKLITQRKF | | | | |
| | | DNLTKAERGGLSELDKAGFIKRQLVETRQITKHVAQILDSRMNTKYDENDKLI | | | | |
| | | REVKVITLKSKLVSDFRKDFQFYKVREINNYHHAHDAYLNAVVGTALIKKYPK | | | | |
| | | LESEFVYGDYKVYDVRKMIAKSEQEIGKATAKYFFYSNIMNFFKTEITLANGEI | | | | |
| | | RKRPLIETNGETGEIVWDKGRDFATVRKVLSMPQVNIVKKTEVQTGGFSKES | | | | |
| | | ILPKGNSDKLIARKKDWDPKKYGGFNSPTVAYSVLVVAKVEKGKSKKLKSVK | | | | |
| | | ELLGITIMERSSFEKNPIGFLEAKGYKEVKKDLIIKLPKYSLFELENGRKRMLAS | | | | |
| | | ASVLHKGNELALPSKYVNFLYLASHYEKLKGSSEDNKQKQLFVEQHKHYLDEI | | | | |
| | | IEQISEFSKRVILADANLDKVLSAYNKHRDKPIREQAENIIHLFTLTNLGASAAF | | | | |
| | | KYFDTTIGRKLYTSTKEVLDATLIHQSITGLYETRIDLSQLGGD | | | | |
| SpyCas9- | Streptococcus | MDKKYSIGLDIGTNSVGWAVITDEYKVPSKKFKVLGNTDRHSIKKNLIGALLF | 9,026 | N863A | H840A | D10A |
| 3var-NRCH | pyogenes | DSGETAEATRLKRTARRRYTRRKNRICYLQEIFSNEMAKVDDSFFHRLEESFL | | | | |
| | | VEEDKKHERHPIFGNIVDEVAYHEKYPTIYHLRKKLVDSTDKADLRLIYLALAH | | | | |
| | | MIKFRGHFLIEGDLNPDNSDVDKLFIQLVQTYNQLFEENPINASGVDAKAILS | | | | |
| | | ARLSKSRRLENLIAQLPGEKKNGLFGNLIALSLGLTPNFKSNFDLAEDAKLQLS | | | | |
| | | KDTYDDDLDNLLAQIGDQYADLFLAAKNLSDAILLSDILRVNTEITKAPLSAS | | | | |
| | | MVKRYDEHHQDLTLLKALVRQQLPEKYKEIFFDQSKNGYAGYIDGGASQEE | | | | |
| | | FYKFIKPILEKMDGTEELLVKLNREDLLRKQRTFDNGIIPHQIHLGELHAILRRQ | | | | |
| | | GDFYPFLKDNREKIEKILTFRIPYYVGPLARGNSRFAWMTRKSEETITPWNFE | | | | |
| | | EVVDKGASAQSFIERMTNFDKNLPNEKVLPKHSLLYEYFTVYNELTKVKYVTE | | | | |
| | | GMRKPAFLSGEQKKAIVDLLFKTNRKVTVKQLKEDYFKKIECFDSVEISGVED | | | | |
| | | RFNASLGTYHDLLKIIKDKDFLDNEENEDILEDIVLTLTLFEDREMIEERLKTYA | | | | |
| | | HLFDDKVMKQLKRLRYTGWGRLSRKLINGIRDKQSGKTILDFLKSDGFANRN | | | | |
| | | FMQLIHDDSLTFKEDIQKAQVSGQGDSLHEHIANLAGSPAIKKGILQTVKVV | | | | |
| | | DELVKVMGGHKPENIVIEMARENQTTQKGQKNSRERMKRIEEGIKELGSQI | | | | |
| | | LKEHPVENTQLQNEKLYLYYLQNGRDMYVDQELDINRLSDYDVDHIVPQSF | | | | |
| | | LKDDSIDNKVLTRSDKNRGKSDNVPSEEVVKKMKNYWRQLLNAKLITQRKF | | | | |
| | | DNLTKAERGGLSELDKAGFIKRQLVETRQITKHVAQILDSRMNTKYDENDKLI | | | | |
| | | REVKVITLKSKLVSDFRKDFQFYKVREINNYHHAHDAYLNAVVGTALIKKYPK | | | | |
| | | LESEFVYGDYKVYDVRKMIAKSEQEIGKATAKYFFYSNIMNFFKTEITLANGEI | | | | |
| | | RKRPLIETNGETGEIVWDKGRDFATVRKVLSMPQVNIVKKTEVQTGGFSKES | | | | |
| | | ILPKGNSDKLIARKKDWDPKKYGGFNSPTVAYSVLVVAKVEKGKSKKLKSVK | | | | |
| | | ELLGITIMERSSFEKNPIDFLEAKGYKEVKKDLIIKLPKYSLFELENGRKRMLAS | | | | |
| | | AGVLQKGNELALPSKYVNFLYLASHYEKLKGSPEDNEQKQLFVEQHKHYLDE | | | | |
| | | IIEQISEFSKRVILADANLDKVLSAYNKHRDKPIREQAENIIHLFTLTNLGAPAA | | | | |
| | | FKYFDTTINRKQYNTTKEVLDATLIRQSITGLYETRIDLSQLGGD | | | | |
| SpyCas9- | Streptococcus | MDKKYSIGLDIGTNSVGWAVITDEYKVPSKKFKVLGNTDRHSIKKNLIGALLF | 9,027 | N863A | H840A | D10A |
| HF1 | pyogenes | DSGETAEATRLKRTARRRYTRRKNRICYLQEIFSNEMAKVDDSFFHRLEESFL | | | | |
| | | VEEDKKHERHPIFGNIVDEVAYHEKYPTIYHLRKKLVDSTDKADLRLIYLALAH | | | | |
| | | MIKFRGHFLIEGDLNPDNSDVDKLFIQLVQTYNQLFEENPINASGVDAKAILS | | | | |
| | | ARLSKSRRLENLIAQLPGEKKNGLFGNLIALSLGLTPNFKSNFDLAEDAKLQLS | | | | |
| | | KDTYDDDLDNLLAQIGDQYADLFLAAKNLSDAILLSDILRVNTEITKAPLSAS | | | | |
| | | MIKRYDEHHQDLTLLKALVRQQLPEKYKEIFFDQSKNGYAGYIDGGASQEEF | | | | |
| | | YKFIKPILEKMDGTEELLVKLNREDLLRKQRTFDNGSIPHQIHLGELHAILRRQ | | | | |
| | | EDFYPFLKDNREKIEKILTFRIPYYVGPLARGNSRFAWMTRKSEETITPWNFE | | | | |
| | | EVVDKGASAQSFIERMTNFDKNLPNEKVLPKHSLLYEYFTVYNELTKVKYVTE | | | | |
| | | GMRKPAFLSGEQKKAIVDLLFKTNRKVTVKQLKEDYFKKIECFDSVEISGVED | | | | |
| | | RFNASLGTYHDLLKIIKDKDFLDNEENEDILEDIVLTLTLFEDREMIEERLKTYA | | | | |
| | | HLFDDKVMKQLKRRRYTGWGRLSRKLINGIRDKQSGKTILDFLKSDGFANR | | | | |
| | | NFMQLIHDDSLTFKEDIQKAQVSGQGDSLHEHIANLAGSPAIKKGILQTVKV | | | | |
| | | VDELVKVMGRHKPENIVIEMARENQTTQKGQKNSRERMKRIEEGIKELGSQ | | | | |
| | | ILKEHPVENTQLQNEKLYLYYLQNGRDMYVDQELDINRLSDYDVDHIVPQSF | | | | |
| | | LKDDSIDNKVLTRSDKNRGKSDNVPSEEVVKKMKNYWRQLLNAKLITQRKF | | | | |
| | | DNLTKAERGGLSELDKAGFIKRQLVETRQITKHVAQILDSRMNTKYDENDKLI | | | | |
| | | REVKVITLKSKLVSDFRKDFQFYKVREINNYHHAHDAYLNAVVGTALIKKYPK | | | | |
| | | LESEFVYGDYKVYDVRKMIAKSEQEIGKATAKYFFYSNIMNFFKTEITLANGEI | | | | |
| | | RKRPLIETNGETGEIVWDKGRDFATVRKVLSMPQVNIVKKTEVQTGGFSKES | | | | |
| | | ILPKRNSDKLIARKKDWDPKKYGGFDSPTVAYSVLVVAKVEKGKSKKLKSVKE | | | | |
| | | LLGITIMERSSFEKNPIDFLEAKGYKEVKKDLIIKLPKYSLFELENGRKRMLASA | | | | |
| | | GELQKGNELALPSKYVNFLYLASHYEKLKGSPEDNEQKQLFVEQHKHYLDEII | | | | |
| | | EQISEFSKRVILADANLDKVLSAYNKHRDKPIREQAENIIHLFTLTNLGAPAAF | | | | |
| | | KYFDTTIDRKRYTSTKEVLDATLIHQSITGLYETRIDLSQLGGD | | | | |
| SpyCas9- | Streptococcus | MDKKYSIGLDIGTNSVGWAVITDEYKVPSKKFKVLGNTDRHSIKKNLIGALLF | 9,028 | N863A | H840A | D10A |
| QQR1 | pyogenes | DSGETAEATRLKRTARRRYTRRKNRICYLQEIFSNEMAKVDDSFFHRLEESFL | | | | |
| | | VEEDKKHERHPIFGNIVDEVAYHEKYPTIYHLRKKLVDSTDKADLRLIYLALAH | | | | |
| | | MIKFRGHFLIEGDLNPDNSDVDKLFIQLVQTYNQLFEENPINASGVDAKAILS | | | | |
| | | ARLSKSRRLENLIAQLPGEKKNGLFGNLIALSLGLTPNFKSNFDLAEDAKLQLS | | | | |
| | | KDTYDDDLDNLLAQIGDQYADLFLAAKNLSDAILLSDILRVNTEITKAPLSAS | | | | |
| | | MIKRYDEHHQDLTLLKALVRQQLPEKYKEIFFDQSKNGYAGYIDGGASQEEF | | | | |
| | | YKFIKPILEKMDGTEELLVKLNREDLLRKQRTFDNGSIPHQIHLGELHAILRRQ | | | | |
| | | EDFYPFLKDNREKIEKILTFRIPYYVGPLARGNSRFAWMTRKSEETITPWNFE | | | | |
| | | EVVDKGASAQSFIERMTNFDKNLPNEKVLPKHSLLYEYFTVYNELTKVKYVTE | | | | |
| | | GMRKPAFLSGEQKKAIVDLLFKTNRKVTVKQLKEDYFKKIECFDSVEISGVED | | | | |
| | | RFNASLGTYHDLLKIIKDKDFLDNEENEDILEDIVLTLTLFEDREMIEERLKTYA | | | | |
| | | HLFDDKVMKQLKRRRYTGWGRLSRKLINGIRDKQSGKTILDFLKSDGFANR | | | | |
| | | NFMQLIHDDSLTFKEDIQKAQVSGQGDSLHEHIANLAGSPAIKKGILQTVKV | | | | |
| | | VDELVKVMGRHKPENIVIEMARENQTTQKGQKNSRERMKRIEEGIKELGSQ | | | | |
| | | ILKEHPVENTQLQNEKLYLYYLQNGRDMYVDQELDINRLSDYDVDHIVPQSF | | | | |
| | | LKDDSIDNKVLTRSDKNRGKSDNVPSEEVVKKMKNYWRQLLNAKLITQRKF | | | | |
| | | DNLTKAERGGLSELDKAGFIKRQLVETRQITKHVAQILDSRMNTKYDENDKLI | | | | |
| | | REVKVITLKSKLVSDFRKDFQFYKVREINNYHHAHDAYLNAVVGTALIKKYPK | | | | |
| | | LESEFVYGDYKVYDVRKMIAKSEQEIGKATAKYFFYSNIMNFFKTEITLANGEI | | | | |
| | | RKRPLIETNGETGEIVWDKGRDFATVRKVLSMPQVNIVKKTEVQTGGFSKES | | | | |
| | | ILPKRNSDKLIARKKDWDPKKYGGFDSPTVAYSVLVVAKVEKGKSKKLKSVKE | | | | |
| | | LLGITIMERSSFEKNPIDFLEAKGYKEVKKDLIIKLPKYSLFELENGRKRMLASA | | | | |
| | | RELQKGNELALPSKYVNFLYLASHYEKLKGSPEDNEQKQLFVEQHKHYLDEII | | | | |
| | | EQISEFSKRVILADAQLDKVLSAYNKHRDKPIREQAENIIHLFTLTNLGAPAAF | | | | |
| | | KYFDTTFKQKQYRSTKEVLDATLIHQSITGLYETRIDLSQLGGD | | | | |
| SpyCas9- | Streptococcus | MDKKYSIGLDIGTNSVGWAVITDEYKVPSKKFKVLGNTDRHSIKKNLIGALLF | 9,029 | N863A | H840A | D10A |
| SpG | pyogenes | DSGETAEATRLKRTARRRYTRRKNRICYLQEIFSNEMAKVDDSFFHRLEESFL | | | | |
| | | VEEDKKHERHPIFGNIVDEVAYHEKYPTIYHLRKKLVDSTDKADLRLIYLALAH | | | | |
| | | MIKFRGHFLIEGDLNPDNSDVDKLFIQLVQTYNQLFEENPINASGVDAKAILS | | | | |
| | | ARLSKSRRLENLIAQLPGEKKNGLFGNLIALSLGLTPNFKSNFDLAEDAKLQLS | | | | |
| | | KDTYDDDLDNLLAQIGDQYADLFLAAKNLSDAILLSDILRVNTEITKAPLSAS | | | | |
| | | MIKRYDEHHQDLTLLKALVRQQLPEKYKEIFFDQSKNGYAGYIDGGASQEEF | | | | |
| | | YKFIKPILEKMDGTEELLVKLNREDLLRKQRTFDNGSIPHQIHLGELHAILRRQ | | | | |
| | | EDFYPFLKDNREKIEKILTFRIPYYVGPLARGNSRFAWMTRKSEETITPWNFE | | | | |
| | | EVVDKGASAQSFIERMTNFDKNLPNEKVLPKHSLLYEYFTVYNELTKVKYVTE | | | | |
| | | GMRKPAFLSGEQKKAIVDLLFKTNRKVTVKQLKEDYFKKIECFDSVEISGVED | | | | |
| | | RFNASLGTYHDLLKIIKDKDFLDNEENEDILEDIVLTLTLFEDREMIEERLKTYA | | | | |
| | | HLFDDKVMKQLKRRRYTGWGRLSRKLINGIRDKQSGKTILDFLKSDGFANR | | | | |
| | | NFMQLIHDDSLTFKEDIQKAQVSGQGDSLHEHIANLAGSPAIKKGILQTVKV | | | | |
| | | VDELVKVMGRHKPENIVIEMARENQTTQKGQKNSRERMKRIEEGIKELGSQ | | | | |
| | | ILKEHPVENTQLQNEKLYLYYLQNGRDMYVDQELDINRLSDYDVDHIVPQSF | | | | |
| | | LKDDSIDNKVLTRSDKNRGKSDNVPSEEVVKKMKNYWRQLLNAKLITQRKF | | | | |
| | | DNLTKAERGGLSELDKAGFIKRQLVETRQITKHVAQILDSRMNTKYDENDKLI | | | | |
| | | REVKVITLKSKLVSDFRKDFQFYKVREINNYHHAHDAYLNAVVGTALIKKYPK | | | | |
| | | LESEFVYGDYKVYDVRKMIAKSEQEIGKATAKYFFYSNIMNFFKTEITLANGEI | | | | |
| | | RKRPLIETNGETGEIVWDKGRDFATVRKVLSMPQVNIVKKTEVQTGGFSKES | | | | |
| | | ILPKRNSDKLIARKKDWDPKKYGGFLWPTVAYSVLVVAKVEKGKSKKLKSVK | | | | |
| | | ELLGITIMERSSFEKNPIDFLEAKGYKEVKKDLIIKLPKYSLFELENGRKRMLAS | | | | |
| | | AKQLQKGNELALPSKYVNFLYLASHYEKLKGSPEDNEQKQLFVEQHKHYLDE | | | | |
| | | IIEQISEFSKRVILADANLDKVLSAYNKHRDKPIREQAENIIHLFTLTNLGAPAA | | | | |
| | | FKYFDTTIDRKQYRSTKEVLDATLIHQSITGLYETRIDLSQLGGD | | | | |
| SpyCas9- | Streptococcus | MDKKYSIGLDIGTNSVGWAVITDEYKVPSKKFKVLGNTDRHSIKKNLIGALLF | 9,030 | N863A | H840A | D10A |
| VQR | pyogenes | DSGETAEATRLKRTARRRYTRRKNRICYLQEIFSNEMAKVDDSFFHRLEESFL | | | | |
| | | VEEDKKHERHPIFGNIVDEVAYHEKYPTIYHLRKKLVDSTDKADLRLIYLALAH | | | | |
| | | MIKFRGHFLIEGDLNPDNSDVDKLFIQLVQTYNQLFEENPINASGVDAKAILS | | | | |
| | | ARLSKSRRLENLIAQLPGEKKNGLFGNLIALSLGLTPNFKSNFDLAEDAKLQLS | | | | |
| | | KDTYDDDLDNLLAQIGDQYADLFLAAKNLSDAILLSDILRVNTEITKAPLSAS | | | | |
| | | MIKRYDEHHQDLTLLKALVRQQLPEKYKEIFFDQSKNGYAGYIDGGASQEEF | | | | |
| | | YKFIKPILEKMDGTEELLVKLNREDLLRKQRTFDNGSIPHQIHLGELHAILRRQ | | | | |
| | | EDFYPFLKDNREKIEKILTFRIPYYVGPLARGNSRFAWMTRKSEETITPWNFE | | | | |
| | | EVVDKGASAQSFIERMTNFDKNLPNEKVLPKHSLLYEYFTVYNELTKVKYVTE | | | | |
| | | GMRKPAFLSGEQKKAIVDLLFKTNRKVTVKQLKEDYFKKIECFDSVEISGVED | | | | |
| | | RFNASLGTYHDLLKIIKDKDFLDNEENEDILEDIVLTLTLFEDREMIEERLKTYA | | | | |
| | | HLFDDKVMKQLKRRRYTGWGRLSRKLINGIRDKQSGKTILDFLKSDGFANR | | | | |
| | | NFMQLIHDDSLTFKEDIQKAQVSGQGDSLHEHIANLAGSPAIKKGILQTVKV | | | | |
| | | VDELVKVMGRHKPENIVIEMARENQTTQKGQKNSRERMKRIEEGIKELGSQ | | | | |
| | | ILKEHPVENTQLQNEKLYLYYLQNGRDMYVDQELDINRLSDYDVDHIVPQSF | | | | |
| | | LKDDSIDNKVLTRSDKNRGKSDNVPSEEVVKKMKNYWRQLLNAKLITQRKF | | | | |
| | | DNLTKAERGGLSELDKAGFIKRQLVETRQITKHVAQILDSRMNTKYDENDKLI | | | | |
| | | REVKVITLKSKLVSDFRKDFQFYKVREINNYHHAHDAYLNAVVGTALIKKYPK | | | | |
| | | LESEFVYGDYKVYDVRKMIAKSEQEIGKATAKYFFYSNIMNFFKTEITLANGEI | | | | |
| | | RKRPLIETNGETGEIVWDKGRDFATVRKVLSMPQVNIVKKTEVQTGGFSKES | | | | |
| | | ILPKRNSDKLIARKKDWDPKKYGGFVSPTVAYSVLVVAKVEKGKSKKLKSVKE | | | | |
| | | LLGITIMERSSFEKNPIDFLEAKGYKEVKKDLIIKLPKYSLFELENGRKRMLASA | | | | |
| | | GELQKGNELALPSKYVNFLYLASHYEKLKGSPEDNEQKQLFVEQHKHYLDEII | | | | |
| | | EQISEFSKRVILADANLDKVLSAYNKHRDKPIREQAENIIHLFTLTNLGAPAAF | | | | |
| | | KYFDTTIDRKQYRSTKEVLDATLIHQSITGLYETRIDLSQLGGD | | | | |
| SpyCas9- | Streptococcus | MDKKYSIGLDIGTNSVGWAVITDEYKVPSKKFKVLGNTDRHSIKKNLIGALLF | 9,031 | N863A | H840A | D10A |
| VRER | pyogenes | DSGETAEATRLKRTARRRYTRRKNRICYLQEIFSNEMAKVDDSFFHRLEESFL | | | | |
| | | VEEDKKHERHPIFGNIVDEVAYHEKYPTIYHLRKKLVDSTDKADLRLIYLALAH | | | | |
| | | MIKFRGHFLIEGDLNPDNSDVDKLFIQLVQTYNQLFEENPINASGVDAKAILS | | | | |
| | | ARLSKSRRLENLIAQLPGEKKNGLFGNLIALSLGLTPNFKSNFDLAEDAKLQLS | | | | |
| | | KDTYDDDLDNLLAQIGDQYADLFLAAKNLSDAILLSDILRVNTEITKAPLSAS | | | | |
| | | MIKRYDEHHQDLTLLKALVRQQLPEKYKEIFFDQSKNGYAGYIDGGASQEEF | | | | |
| | | YKFIKPILEKMDGTEELLVKLNREDLLRKQRTFDNGSIPHQIHLGELHAILRRQ | | | | |
| | | EDFYPFLKDNREKIEKILTFRIPYYVGPLARGNSRFAWMTRKSEETITPWNFE | | | | |
| | | EVVDKGASAQSFIERMTNFDKNLPNEKVLPKHSLLYEYFTVYNELTKVKYVTE | | | | |
| | | GMRKPAFLSGEQKKAIVDLLFKTNRKVTVKQLKEDYFKKIECFDSVEISGVED | | | | |
| | | RFNASLGTYHDLLKIIKDKDFLDNEENEDILEDIVLTLTLFEDREMIEERLKTYA | | | | |
| | | HLFDDKVMKQLKRRRYTGWGRLSRKLINGIRDKQSGKTILDFLKSDGFANR | | | | |
| | | NFMQLIHDDSLTFKEDIQKAQVSGQGDSLHEHIANLAGSPAIKKGILQTVKV | | | | |
| | | VDELVKVMGRHKPENIVIEMARENQTTQKGQKNSRERMKRIEEGIKELGSQ | | | | |
| | | ILKEHPVENTQLQNEKLYLYYLQNGRDMYVDQELDINRLSDYDVDHIVPQSF | | | | |
| | | LKDDSIDNKVLTRSDKNRGKSDNVPSEEVVKKMKNYWRQLLNAKLITQRKF | | | | |
| | | DNLTKAERGGLSELDKAGFIKRQLVETRQITKHVAQILDSRMNTKYDENDKLI | | | | |
| | | REVKVITLKSKLVSDFRKDFQFYKVREINNYHHAHDAYLNAVVGTALIKKYPK | | | | |
| | | LESEFVYGDYKVYDVRKMIAKSEQEIGKATAKYFFYSNIMNFFKTEITLANGEI | | | | |
| | | RKRPLIETNGETGEIVWDKGRDFATVRKVLSMPQVNIVKKTEVQTGGFSKES | | | | |
| | | ILPKRNSDKLIARKKDWDPKKYGGFVSPTVAYSVLVVAKVEKGKSKKLKSVKE | | | | |
| | | LLGITIMERSSFEKNPIDFLEAKGYKEVKKDLIIKLPKYSLFELENGRKRMLASA | | | | |
| | | RELQKGNELALPSKYVNFLYLASHYEKLKGSPEDNEQKQLFVEQHKHYLDEII | | | | |
| | | EQISEFSKRVILADANLDKVLSAYNKHRDKPIREQAENIIHLFTLTNLGAPAAF | | | | |
| | | KYFDTTIDRKEYRSTKEVLDATLIHQSITGLYETRIDLSQLGGD | | | | |
| SpyCas9- | Streptococcus | MDKKYSIGLDIGTNSVGWAVITDEYKVPSKKFKVLGNTDRHSIKKNLIGALLF | 9,032 | N863A | H840A | D10A |
| xCas | pyogenes | DSGETAEATRLKRTARRRYTRRKNRICYLQEIFSNEMAKVDDSFFHRLEESFL | | | | |
| | | VEEDKKHERHPIFGNIVDEVAYHEKYPTIYHLRKKLVDSTDKADLRLIYLALAH | | | | |
| | | MIKFRGHFLIEGDLNPDNSDVDKLFIQLVQTYNQLFEENPINASGVDAKAILS | | | | |
| | | ARLSKSRRLENLIAQLPGEKKNGLFGNLIALSLGLTPNFKSNFDLAEDTKLQLS | | | | |
| | | KDTYDDDLDNLLAQIGDQYADLFLAAKNLSDAILLSDILRVNTEITKAPLSAS | | | | |
| | | MIKLYDEHHQDLTLLKALVRQQLPEKYKEIFFDQSKNGYAGYIDGGASQEEF | | | | |
| | | YKFIKPILEKMDGTEELLVKLNREDLLRKQRTFDNGIIPHQIHLGELHAILRRQE | | | | |
| | | DFYPFLKDNREKIEKILTFRIPYYVGPLARGNSRFAWMTRKSEETITPWNFEK | | | | |
| | | VVDKGASAQSFIERMTNFDKNLPNEKVLPKHSLLYEYFTVYNELTKVKYVTE | | | | |
| | | GMRKPAFLSGDQKKAIVDLLFKTNRKVTVKQLKEDYFKKIECFDSVEISGVED | | | | |
| | | RFNASLGTYHDLLKIIKDKDFLDNEENEDILEDIVLTLTLFEDREMIEERLKTYA | | | | |
| | | HLFDDKVMKQLKRRRYTGWGRLSRKLINGIRDKQSGKTILDFLKSDGFANR | | | | |
| | | NFIQLIHDDSLTFKEDIQKAQVSGQGDSLHEHIANLAGSPAIKKGILQTVKVV | | | | |
| | | DELVKVMGRHKPENIVIEMARENQTTQKGQKNSRERMKRIEEGIKELGSQI | | | | |
| | | LKEHPVENTQLQNEKLYLYYLQNGRDMYVDQELDINRLSDYDVDHIVPQSF | | | | |
| | | LKDDSIDNKVLTRSDKNRGKSDNVPSEEVVKKMKNYWRQLLNAKLITQRKF | | | | |
| | | DNLTKAERGGLSELDKAGFIKRQLVETRQITKHVAQILDSRMNTKYDENDKLI | | | | |
| | | REVKVITLKSKLVSDFRKDFQFYKVREINNYHHAHDAYLNAVVGTALIKKYPK | | | | |
| | | LESEFVYGDYKVYDVRKMIAKSEQEIGKATAKYFFYSNIMNFFKTEITLANGEI | | | | |
| | | RKRPLIETNGETGEIVWDKGRDFATVRKVLSMPQVNIVKKTEVQTGGFSKES | | | | |
| | | ILPKRNSDKLIARKKDWDPKKYGGFDSPTVAYSVLVVAKVEKGKSKKLKSVKE | | | | |
| | | LLGITIMERSSFEKNPIDFLEAKGYKEVKKDLIIKLPKYSLFELENGRKRMLASA | | | | |
| | | GVLQKGNELALPSKYVNFLYLASHYEKLKGSPEDNEQKQLFVEQHKHYLDEII | | | | |
| | | EQISEFSKRVILADANLDKVLSAYNKHRDKPIREQAENIIHLFTLTNLGAPAAF | | | | |
| | | KYFDTTIDRKRYTSTKEVLDATLIHQSITGLYETRIDLSQLGGD | | | | |
| SpyCas9- | Streptococcus | MDKKYSIGLDIGTNSVGWAVITDEYKVPSKKFKVLGNTDRHSIKKNLIGALLF | 9,033 | N863A | H840A | D10A |
| xCas-NG | pyogenes | DSGETAEATRLKRTARRRYTRRKNRICYLQEIFSNEMAKVDDSFFHRLEESFL | | | | |
| | | VEEDKKHERHPIFGNIVDEVAYHEKYPTIYHLRKKLVDSTDKADLRLIYLALAH | | | | |
| | | MIKFRGHFLIEGDLNPDNSDVDKLFIQLVQTYNQLFEENPINASGVDAKAILS | | | | |
| | | ARLSKSRRLENLIAQLPGEKKNGLFGNLIALSLGLTPNFKSNFDLAEDTKLQLS | | | | |
| | | KDTYDDDLDNLLAQIGDQYADLFLAAKNLSDAILLSDILRVNTEITKAPLSAS | | | | |
| | | MIKLYDEHHQDLTLLKALVRQQLPEKYKEIFFDQSKNGYAGYIDGGASQEEF | | | | |
| | | YKFIKPILEKMDGTEELLVKLNREDLLRKQRTFDNGIIPHQIHLGELHAILRRQE | | | | |
| | | DFYPFLKDNREKIEKILTFRIPYYVGPLARGNSRFAWMTRKSEETITPWNFEK | | | | |
| | | VVDKGASAQSFIERMTNFDKNLPNEKVLPKHSLLYEYFTVYNELTKVKYVTE | | | | |
| | | GMRKPAFLSGDQKKAIVDLLFKTNRKVTVKQLKEDYFKKIECFDSVEISGVED | | | | |
| | | RFNASLGTYHDLLKIIKDKDFLDNEENEDILEDIVLTLTLFEDREMIEERLKTYA | | | | |
| | | HLFDDKVMKQLKRRRYTGWGRLSRKLINGIRDKQSGKTILDFLKSDGFANR | | | | |
| | | NFIQLIHDDSLTFKEDIQKAQVSGQGDSLHEHIANLAGSPAIKKGILQTVKVV | | | | |
| | | DELVKVMGRHKPENIVIEMARENQTTQKGQKNSRERMKRIEEGIKELGSQI | | | | |
| | | LKEHPVENTQLQNEKLYLYYLQNGRDMYVDQELDINRLSDYDVDHIVPQSF | | | | |
| | | LKDDSIDNKVLTRSDKNRGKSDNVPSEEVVKKMKNYWRQLLNAKLITQRKF | | | | |
| | | DNLTKAERGGLSELDKAGFIKRQLVETRQITKHVAQILDSRMNTKYDENDKLI | | | | |
| | | REVKVITLKSKLVSDFRKDFQFYKVREINNYHHAHDAYLNAVVGTALIKKYPK | | | | |
| | | LESEFVYGDYKVYDVRKMIAKSEQEIGKATAKYFFYSNIMNFFKTEITLANGEI | | | | |
| | | RKRPLIETNGETGEIVWDKGRDFATVRKVLSMPQVNIVKKTEVQTGGFSKES | | | | |
| | | IRPKRNSDKLIARKKDWDPKKYGGFVSPTVAYSVLVVAKVEKGKSKKLKSVKE | | | | |
| | | LLGITIMERSSFEKNPIDFLEAKGYKEVKKDLIIKLPKYSLFELENGRKRMLASA | | | | |
| | | RFLQKGNELALPSKYVNFLYLASHYEKLKGSPEDNEQKQLFVEQHKHYLDEII | | | | |
| | | EQISEFSKRVILADANLDKVLSAYNKHRDKPIREQAENIIHLFTLTNLGAPRAF | | | | |
| | | KYFDTTIDRKVYRSTKEVLDATLIHQSITGLYETRIDLSQLGGD | | | | |
| St1Cas9- | Streptococcus | MSDLVLGLDIGIGSVGVGILNKVTGEIIHKNSRIFPAAQAENNLVRRTNRQG | 9,034 | N622A | H599A | D9A |
| CNRZ1066 | thermophilus | RRLARRKKHRRVRLNRLFEESGLITDFTKISINLNPYQLRVKGLTDELSNEELFI | | | | |
| | | ALKNMVKHRGISYLDDASDDGNSSVGDYAQIVKENSKQLETKTPGQIQLER | | | | |
| | | YQTYGQLRGDFTVEKDGKKHRLINVFPTSAYRSEALRILQTQQEFNPQITDEF | | | | |
| | | INRYLEILTGKRKYYHGPGNEKSRTDYGRYRTSGETLDNIFGILIGKCTFYPDEF | | | | |
| | | RAAKASYTAQEFNLLNDLNNLTVPTETKKLSKEQKNQIINYVKNEKAMGPAK | | | | |
| | | LFKYIAKLLSCDVADIKGYRIDKSGKAEIHTFEAYRKMKTLETLDIEQMDRETL | | | | |
| | | DKLAYVLTLNTEREGIQEALEHEFADGSFSQKQVDELVQFRKANSSIFGKGW | | | | |
| | | HNFSVKLMMELIPELYETSEEQMTILTRLGKQKTTSSSNKTKYIDEKLLTEEIY | | | | |
| | | NPVVAKSVRQAIKIVNAAIKEYGDFDNIVIEMARETNEDDEKKAIQKIQKAN | | | | |
| | | KDEKDAAMLKAANQYNGKAELPHSVFHGHKQLATKIRLWHQQGERCLYT | | | | |
| | | GKTISIHDLINNSNQFEVDHILPLSITFDDSLANKVLVYATANQEKGQRTPYQ | | | | |
| | | ALDSMDDAWSFRELKAFVRESKTLSNKKKEYLLTEEDISKFDVRKKFIERNLV | | | | |
| | | DTRYASRVVLNALQEHFRAHKIDTKVSVVRGQFTSQLRRHWGIEKTRDTYH | | | | |
| | | HHAVDALIIAASSQLNLWKKQKNTLVSYSEEQLLDIETGELISDDEYKESVFKA | | | | |
| | | PYQHFVDTLKSKEFEDSILFSYQVDSKFNRKISDATIYATRQAKVGKDKKDET | | | | |
| | | YVLGKIKDIYTQDGYDAFMKIYKKDKSKFLMYRHDPQTFEKVIEPILENYPNK | | | | |
| | | QMNEKGKEVPCNPFLKYKEEHGYIRKYSKKGNGPEIKSLKYYDSKLLGNPIDI | | | | |
| | | TPENSKNKVVLQSLKPWRTDVYFNKATGKYEILGLKYADLQFEKGTGTYKIS | | | | |
| | | QEKYNDIKKKEGVDSDSEFKFTLYKNDLLLVKDTETKEQQLFRFLSRTLPKQK | | | | |
| | | HYVELKPYDKQKFEGGEALIKVLGNVANGGQCIKGLAKSNISIYKVRTDVLG | | | | |
| | | NQHIIKNEGDKPKLDF | | | | |
| St1Cas9- | Streptococcus | MSDLVLGLDIGIGSVGVGILNKVTGEIIHKNSRIFPAAQAENNLVRRTNRQG | 9,035 | N622A | H599A | D9A |
| LMG1831 | thermophilus | RRLARRKKHRRVRLNRLFEESGLITDFTKISINLNPYQLRVKGLTDELSNEELFI | | | | |
| | | ALKNMVKHRGISYLDDASDDGNSSVGDYAQIVKENSKQLETKTPGQIQLER | | | | |
| | | YQTYGQLRGDFTVEKDGKKHRLINVFPTSAYRSEALRILQTQQEFNPQITDEF | | | | |
| | | INRYLEILTGKRKYYHGPGNEKSRTDYGRYRTSGETLDNIFGILIGKCTFYPDEF | | | | |
| | | RAAKASYTAQEFNLLNDLNNLTVPTETKKLSKEQKNQIINYVKNEKAMGPAK | | | | |
| | | LFKYIAKLLSCDVADIKGYRIDKSGKAEIHTFEAYRKMKTLETLDIEQMDRETL | | | | |
| | | DKLAYVLTLNTEREGIQEALEHEFADGSFSQKQVDELVQFRKANSSIFGKGW | | | | |
| | | HNFSVKLMMELIPELYETSEEQMTILTRLGKQKTTSSSNKTKYIDEKLLTEEIY | | | | |
| | | NPVVAKSVRQAIKIVNAAIKEYGDFDNIVIEMARETNEDDEKKAIQKIQKAN | | | | |
| | | KDEKDAAMLKAANQYNGKAELPHSVFHGHKQLATKIRLWHQQGERCLYT | | | | |
| | | GKTISIHDLINNSNQFEVDHILPLSITFDDSLANKVLVYATANQEKGQRTPYQ | | | | |
| | | ALDSMDDAWSFRELKAFVRESKTLSNKKKEYLLTEEDISKFDVRKKFIERNLV | | | | |
| | | DTRYASRVVLNALQEHFRAHKIDTKVSVVRGQFTSQLRRHWGIEKTRDTYH | | | | |
| | | HHAVDALIIAASSQLNLWKKQKNTLVSYSEEQLLDIETGELISDDEYKESVFKA | | | | |
| | | PYQHFVDTLKSKEFEDSILFSYQVDSKFNRKISDATIYATRQAKVGKDKKDET | | | | |
| | | YVLGKIKDIYTQDGYDAFMKIYKKDKSKFLMYRHDPQTFEKVIEPILENYPNK | | | | |
| | | QMNEKGKEVPCNPFLKYKEEHGYIRKYSKKGNGPEIKSLKYYDSKLLGNPIDI | | | | |
| | | TPENSKNKVVLQSLKPWRTDVYFNKNTGKYEILGLKYADLQFEKKTGTYKISQ | | | | |
| | | EKYNGIMKEEGVDSDSEFKFTLYKNDLLLVKDTETKEQQLFRFLSRTMPNVK | | | | |
| | | YYVELKPYSKDKFEKNESLIEILGSADKSGRCIKGLGKSNISIYKVRTDVLGNQH | | | | |
| | | IIKNEGDKPKLDF | | | | |
| St1Cas9- | Streptococcus | MSDLVLGLDIGIGSVGVGILNKVTGEIIHKNSRIFPAAQAENNLVRRTNRQG | 9,036 | N622A | H599A | D9A |
| MTH17CL3 | thermophilus | RRLARRKKHRRVRLNRLFEESGLITDFTKISINLNPYQLRVKGLTDELSNEELFI | | | | |
| 96 | | ALKNMVKHRGISYLDDASDDGNSSVGDYAQIVKENSKQLETKTPGQIQLER | | | | |
| | | YQTYGQLRGDFTVEKDGKKHRLINVFPTSAYRSEALRILQTQQEFNPQITDEF | | | | |
| | | INRYLEILTGKRKYYHGPGNEKSRTDYGRYRTSGETLDNIFGILIGKCTFYPDEF | | | | |
| | | RAAKASYTAQEFNLLNDLNNLTVPTETKKLSKEQKNQIINYVKNEKAMGPAK | | | | |
| | | LFKYIAKLLSCDVADIKGYRIDKSGKAEIHTFEAYRKMKTLETLDIEQMDRETL | | | | |
| | | DKLAYVLTLNTEREGIQEALEHEFADGSFSQKQVDELVQFRKANSSIFGKGW | | | | |
| | | HNFSVKLMMELIPELYETSEEQMTILTRLGKQKTTSSSNKTKYIDEKLLTEEIY | | | | |
| | | NPVVAKSVRQAIKIVNAAIKEYGDFDNIVIEMARETNEDDEKKAIQKIQKAN | | | | |
| | | KDEKDAAMLKAANQYNGKAELPHSVFHGHKQLATKIRLWHQQGERCLYT | | | | |
| | | GKTISIHDLINNSNQFEVDHILPLSITFDDSLANKVLVYATANQEKGQRTPYQ | | | | |
| | | ALDSMDDAWSFRELKAFVRESKTLSNKKKEYLLTEEDISKFDVRKKFIERNLV | | | | |
| | | DTRYASRVVLNALQEHFRAHKIDTKVSVVRGQFTSQLRRHWGIEKTRDTYH | | | | |
| | | HHAVDALIIAASSQLNLWKKQKNTLVSYSEDQLLDIETGELISDDEYKESVFK | | | | |
| | | APYQHFVDTLKSKEFEDSILFSYQVDSKFNRKISDATIYATRQAKVGKDKADE | | | | |
| | | TYVLGKIKDIYTQDGYDAFMKIYKKDKSKFLMYRHDPQTFEKVIEPILENYPN | | | | |
| | | KQINEKGKEVPCNPFLKYKEEHGYIRKYSKKGNGPEIKSLKYYDSKLGNHIDIT | | | | |
| | | PKDSNNKVVLQSLKPWRTDVYFNKNTGKYEILGLKYSDMQFEKGTGKYSISK | | | | |
| | | EQYENIKVREGVDENSEFKFTLYKNDLLLLKDSENGEQILLRFTSRNDTSKHYV | | | | |
| | | ELKPYNRQKFEGSEYLIKSLGTVAKGGQCIKGLGKSNISIYKVRTDVLGNQHII | | | | |
| | | KNEGDKPKLDF | | | | |
| St1Cas9- | Streptococcus | MSDLVLGLDIGIGSVGVGILNKVTGEIIHKNSRIFPAAQAENNLVRRTNRQG | 9,037 | N622A | H599A | D9A |
| TH1477 | thermophilus | RRLARRKKHRRVRLNRLFEESGLITDFTKISINLNPYQLRVKGLTDELSNEELFI | | | | |
| | | ALKNMVKHRGISYLDDASDDGNSSVGDYAQIVKENSKQLETKTPGQIQLER | | | | |
| | | YQTYGQLRGDFTVEKDGKKHRLINVFPTSAYRSEALRILQTQQEFNPQITDEF | | | | |
| | | INRYLEILTGKRKYYHGPGNEKSRTDYGRYRTSGETLDNIFGILIGKCTFYPDEF | | | | |
| | | RAAKASYTAQEFNLLNDLNNLTVPTETKKLSKEQKNQIINYVKNEKAMGPAK | | | | |
| | | LFKYIAKLLSCDVADIKGYRIDKSGKAEIHTFEAYRKMKTLETLDIEQMDRETL | | | | |
| | | DKLAYVLTLNTEREGIQEALEHEFADGSFSQKQVDELVQFRKANSSIFGKGW | | | | |
| | | HNFSVKLMMELIPELYETSEEQMTILTRLGKQKTTSSSNKTKYIDEKLLTEEIY | | | | |
| | | NPVVAKSVRQAIKIVNAAIKEYGDFDNIVIEMARETNEDDEKKAIQKIQKAN | | | | |
| | | KDEKDAAMLKAANQYNGKAELPHSVFHGHKQLATKIRLWHQQGERCLYT | | | | |
| | | GKTISIHDLINNSNQFEVDHILPLSITFDDSLANKVLVYATANQEKGQRTPYQ | | | | |
| | | ALDSMDDAWSFRELKAFVRESKTLSNKKKEYLLTEEDISKFDVRKKFIERNLV | | | | |
| | | DTRYASRVVLNALQEHFRAHKIDTKVSVVRGQFTSQLRRHWGIEKTRDTYH | | | | |
| | | HHAVDALIIAASSQLNLWKKQKNTLVSYSEDQLLDIETGELISDDEYKESVFK | | | | |
| | | APYQHFVDTLKSKEFEDSILFSYQVDSKFNRKISDATIYATRQAKVGKDKADE | | | | |
| | | TYVLGKIKDIYTQDGYDAFMKIYKKDKSKFLMYRHDPQTFEKVIEPILENYPN | | | | |
| | | KQINEKGKEVPCNPFLKYKEEHGYIRKYSKKGNGPEIKSLKYYDSKLGNHIDIT | | | | |
| | | PKDSNNKVVLQSLKPWRTDVYFNKNTGKYEILGLKYSDMQFEKGTGKYSISK | | | | |
| | | EQYENIKVREGVDENSEFKFTLYKNDLLLLKDSENGEQILLRFTSRNDTSKHYV | | | | |
| | | ELKPYNRQKFEGSEYLIKSLGTVVKGGRCIKGLGKSNISIYKVRTDVLGNQHIIK | | | | |
| | | NEGDKPKLDF | | | | |
| sRGN3.1 | Staphylococcus | MNQKFILGLDIGITSVGYGLIDYETKNIIDAGVRLFPEANVENNEGRRSKRGS | 9,038 | N585A | H562A | D10A |
| | spp. | RRLKRRRIHRLERVKLLLTEYDLINKEQIPTSNNPYQIRVKGLSEILSKDELAIAL | | | | |
| | | LHLAKRRGIHNVDVAADKEETASDSLSTKDQINKNAKFLESRYVCELQKERLE | | | | |
| | | NEGHVRGVENRFLTKDIVREAKKIIDTQMQYYPEIDETFKEKYISLVETRREYF | | | | |
| | | EGPGQGSPFGWNGDLKKWYEMLMGHCTYFPQELRSVKYAYSADLFNALN | | | | |
| | | DLNNLIIQRDNSEKLEYHEKYHIIENVFKQKKKPTLKQIAKEIGVNPEDIKGYRI | | | | |
| | | TKSGTPEFTSFKLFHDLKKVVKDHAILDDIDLLNQIAEILTIYQDKDSIVAELGQ | | | | |
| | | LEYLMSEADKQSISELTGYTGTHSLSLKCMNMIIDELWHSSMNQMEVFTYL | | | | |
| | | NMRPKKYELKGYQRIPTDMIDDAILSPVVKRTFIQSINVINKVIEKYGIPEDIIIE | | | | |
| | | LARENNSDDRKKFINNLQKKNEATRKRINEIIGQTGNQNAKRIVEKIRLHDQ | | | | |
| | | QEGKCLYSLESIPLEDLLNNPNHYEVDHIIPRSVSFDNSYHNKVLVKQSENSK | | | | |
| | | KSNLTPYQYFNSGKSKLSYNQFKQHILNLSKSQDRISKKKKEYLLEERDINKFE | | | | |
| | | VQKEFINRNLVDTRYATRELTNYLKAYFSANNMNVKVKTINGSFTDYLRKV | | | | |
| | | WKFKKERNHGYKHHAEDALIIANADFLFKENKKLKAVNSVLEKPEIETKQLDI | | | | |
| | | QVDSEDNYSEMFIIPKQVQDIKDFRNFKYSHRVDKKPNRQLINDTLYSTRKK | | | | |
| | | DNSTYIVQTIKDIYAKDNTTLKKQFDKSPEKFLMYQHDPRTFEKLEVIMKQYA | | | | |
| | | NEKNPLAKYHEETGEYLTKYSKKNNGPIVKSLKYIGNKLGSHLDVTHQFKSST | | | | |
| | | KKLVKLSIKNYRFDVYLTEKGYKFVTIAYLNVFKKDNYYYIPKDKYQELKEKKKI | | | | |
| | | KDTDQFIASFYKNDLIKLNGDLYKIIGVNSDDRNIIELDYYDIKYKDYCEINNIK | | | | |
| | | GEPRIKKTIGKKTESIEKFTTDVLGNLYLHSTEKAPQLIFKRGL | | | | |
| sRGN3.3 | Staphylococcus | MNQKFILGLDIGITSVGYGLIDYETKNIIDAGVRLFPEANVENNEGRRSKRGS | 9,039 | N585A | H562A | D10A |
| | spp. | RRLKRRRIHRLERVKLLLTEYDLINKEQIPTSNNPYQIRVKGLSEILSKDELAIAL | | | | |
| | | LHLAKRRGIHNVDVAADKEETASDSLSTKDQINKNAKFLESRYVCELQKERLE | | | | |
| | | NEGHVRGVENRFLTKDIVREAKKIIDTQMQYYPEIDETFKEKYISLVETRREYF | | | | |
| | | EGPGQGSPFGWNGDLKKWYEMLMGHCTYFPQELRSVKYAYSADLFNALN | | | | |
| | | DLNNLIIQRDNSEKLEYHEKYHIIENVFKQKKKPTLKQIAKEIGVNPEDIKGYRI | | | | |
| | | TKSGTPEFTSFKLFHDLKKVVKDHAILDDIDLLNQIAEILTIYQDKDSIVAELGQ | | | | |
| | | LEYLMSEADKQSISELTGYTGTHSLSLKCMNMIIDELWHSSMNQMEVFTYL | | | | |
| | | NMRPKKYELKGYQRIPTDMIDDAILSPVVKRTFIQSINVINKVIEKYGIPEDIIIE | | | | |
| | | LARENNSDDRKKFINNLQKKNEATRKRINEIIGQTGNQNAKRIVEKIRLHDQ | | | | |
| | | QEGKCLYSLESIPLEDLLNNPNHYEVDHIIPRSVSFDNSYHNKVLVKQSENSK | | | | |
| | | KSNLTPYQYFNSGKSKLSYNQFKQHILNLSKSQDRISKKKKEYLLEERDINKFE | | | | |
| | | VQKEFINRNLVDTRYATRELTSYLKAYFSANNMDVKVKTINGSFTNHLRKV | | | | |
| | | WRFDKYRNHGYKHHAEDALIIANADFLFKENKKLQNTNKILEKPTIENNTKK | | | | |
| | | VTVEKEEDYNNVFETPKLVEDIKQYRDYKFSHRVDKKPNRQLINDTLYSTRM | | | | |
| | | KDEHDYIVQTITDIYGKDNTNLKKQFNKNPEKFLMYQNDPKTFEKLSIIMKQ | | | | |
| | | YSDEKNPLAKYYEETGEYLTKYSKKNNGPIVKKIKLLGNKVGNHLDVTNKYEN | | | | |
| | | STKKLVKLSIKNYRFDVYLTEKGYKFVTIAYLNVFKKDNYYYIPKDKYQELKEKK | | | | |
| | | KIKDTDQFIASFYKNDLIKLNGDLYKIIGVNSDDRNIIELDYYDIKYKDYCEINNI | | | | |
| | | KGEPRIKKTIGKKTESIEKFTTDVLGNLYLHSTEKAPQLIFKRGL | | | | |

In some embodiments, a Cas protein requires a protospacer adjacent motif (PAM) to be present in or adjacent to a target DNA sequence for the Cas protein to bind and/or function. In some embodiments, the PAM is or comprises, from 5′ to 3′, NGG, YG, NNGRRT, NNNRRT, NGA, TYCV, TATV, NTTN, or NNNGATT, where N stands for any nucleotide, Y stands for C or T, R stands for A or G, and V stands for A or C or G. In some embodiments, a Cas protein is a protein listed in Table 7 or 8. In some embodiments, a Cas protein comprises one or more mutations altering its PAM. In some embodiments, a Cas protein comprises E1369R, E1449H, and R1556A mutations or analogous substitutions to the amino acids corresponding to said positions. In some embodiments, a Cas protein comprises E782K, N968K, and R1015H mutations or analogous substitutions to the amino acids corresponding to said positions. In some embodiments, a Cas protein comprises D1135V, R1335Q, and T1337R mutations or analogous substitutions to the amino acids corresponding to said positions. In some embodiments, a Cas protein comprises S542R and K607R mutations or analogous substitutions to the amino acids corresponding to said positions. In some embodiments, a Cas protein comprises S542R, K548V, and N552R mutations or analogous substitutions to the amino acids corresponding to said positions. Exemplary advances in the engineering of Cas enzymes to recognize altered PAM sequences are reviewed in Collias et al Nature Communications 12:555 (2021), incorporated herein by reference in its entirety.

In some embodiments, the Cas protein is catalytically active and cuts one or both strands of the target DNA site. In some embodiments, cutting the target DNA site is followed by formation of an alteration, e.g., an insertion or deletion, e.g., by the cellular repair machinery.

In some embodiments, the Cas protein is modified to deactivate or partially deactivate the nuclease, e.g., nuclease-deficient Cas9. Whereas wild-type Cas9 generates double-strand breaks (DSBs) at specific DNA sequences targeted by a gRNA, a number of CRISPR endonucleases having modified functionalities are available, for example: a “nickase” version of Cas9 that has been partially deactivated generates only a single-strand break; a catalytically inactive Cas9 (“dCas9”) does not cut target DNA. In some embodiments, dCas9 binding to a DNA sequence may interfere with transcription at that site by steric hindrance. In some embodiments, dCas9 binding to an anchor sequence may interfere with (e.g., decrease or prevent) genomic complex (e.g., ASMC) formation and/or maintenance. In some embodiments, a DNA-binding domain comprises a catalytically inactive Cas9, e.g., dCas9. Many catalytically inactive Cas9 proteins are known in the art. In some embodiments, dCas9 comprises mutations in each endonuclease domain of the Cas protein, e.g., D10A and H840A or N863A mutations. In some embodiments, a catalytically inactive or partially inactive CRISPR/Cas domain comprises a Cas protein comprising one or more mutations, e.g., one or more of the mutations listed in Table 7. In some embodiments, a Cas protein described on a given row of Table 7 comprises one, two, three, or all of the mutations listed in the same row of Table 7. In some embodiments, a Cas protein, e.g., not described in Table 7, comprises one, two, three, or all of the mutations listed in a row of Table 7 or a corresponding mutation at a corresponding site in that Cas protein.

In some embodiments, a catalytically inactive, e.g., dCas9, or partially deactivated Cas9 protein comprises a D11 mutation (e.g., D11A mutation) or an analogous substitution to the amino acid corresponding to said position. In some embodiments, a catalytically inactive Cas9 protein, e.g., dCas9, or partially deactivated Cas9 protein comprises a H969 mutation (e.g., H969A mutation) or an analogous substitution to the amino acid corresponding to said position. In some embodiments, a catalytically inactive Cas9 protein, e.g., dCas9, or partially deactivated Cas9 protein comprises a N995 mutation (e.g., N995A mutation) or an analogous substitution to the amino acid corresponding to said position. In some embodiments, a catalytically inactive Cas9 protein, e.g., dCas9, comprises mutations at one, two, or three of positions D11, H969, and N995 (e.g., D11A, H969A, and N995A mutations) or analogous substitutions to the amino acids corresponding to said positions.

In some embodiments, a catalytically inactive Cas9 protein, e.g., dCas9, or partially deactivated Cas9 protein comprises a D10 mutation (e.g., a D10A mutation) or an analogous substitution to the amino acid corresponding to said position. In some embodiments, a catalytically inactive Cas9 protein, e.g., dCas9, or partially deactivated Cas9 protein comprises a H557 mutation (e.g., a H557A mutation) or an analogous substitution to the amino acid corresponding to said position. In some embodiments, a catalytically inactive Cas9 protein, e.g., dCas9, comprises a D10 mutation (e.g., a D10A mutation) and a H557 mutation (e.g., a H557A mutation) or analogous substitutions to the amino acids corresponding to said positions.

In some embodiments, a catalytically inactive Cas9 protein, e.g., dCas9, or partially deactivated Cas9 protein comprises a D839 mutation (e.g., a D839A mutation) or an analogous substitution to the amino acid corresponding to said position. In some embodiments, a catalytically inactive Cas9 protein, e.g., dCas9, or partially deactivated Cas9 protein comprises a H840 mutation (e.g., a H840A mutation) or an analogous substitution to the amino acid corresponding to said position. In some embodiments, a catalytically inactive Cas9 protein, e.g., dCas9, or partially deactivated Cas9 protein comprises a N863 mutation (e.g., a N863A mutation) or an analogous substitution to the amino acid corresponding to said position. In some embodiments, a catalytically inactive Cas9 protein, e.g., dCas9, comprises a D10 mutation (e.g., D10A), a D839 mutation (e.g., D839A), a H840 mutation (e.g., H840A), and a N863 mutation (e.g., N863A) or analogous substitutions to the amino acids corresponding to said positions. In some embodiments, a catalytically inactive Cas9 protein, e.g., dCas9, or partially deactivated Cas9 protein comprises a E993 mutation (e.g., a E993A mutation) or an analogous substitution to the amino acid corresponding to said position.

In some embodiments, a catalytically inactive Cas9 protein, e.g., dCas9, or partially deactivated Cas9 protein comprises a D917 mutation (e.g., a D917A mutation) or an analogous substitution to the amino acid corresponding to said position. In some embodiments, a catalytically inactive Cas9 protein, e.g., dCas9, or partially deactivated Cas9 protein comprises a a E1006 mutation (e.g., a E1006A mutation) or an analogous substitution to the amino acid corresponding to said position. In some embodiments, a catalytically inactive Cas9 protein, e.g., dCas9, or partially deactivated Cas9 protein comprises a D1255 mutation (e.g., a D1255A mutation) or an analogous substitution to the amino acid corresponding to said position. In some embodiments, a catalytically inactive Cas9 protein, e.g., dCas9, comprises a D917 mutation (e.g., D917A), a E1006 mutation (e.g., E1006A), and a D1255 mutation (e.g., D1255A) or analogous substitutions to the amino acids corresponding to said positions.

In some embodiments, a catalytically inactive Cas9 protein, e.g., dCas9, or partially deactivated Cas9 protein comprises a D16 mutation (e.g., a D16A mutation) or an analogous substitution to the amino acid corresponding to said position. In some embodiments, a catalytically inactive Cas9 protein, e.g., dCas9, or partially deactivated Cas9 protein comprises a D587 mutation (e.g., a D587A mutation) or an analogous substitution to the amino acid corresponding to said position. In some embodiments, a partially deactivated Cas domain has nickase activity. In some embodiments, a partially deactivated Cas9 domain is a Cas9 nickase domain. In some embodiments, the catalytically inactive Cas domain or dead Cas domain produces no detectable double strand break formation. In some embodiments, a catalytically inactive Cas9 protein, e.g., dCas9, or partially deactivated Cas9 protein comprises a H588 mutation (e.g., a H588A mutation) or an analogous substitution to the amino acid corresponding to said position. In some embodiments, a catalytically inactive Cas9 protein, e.g., dCas9, or partially deactivated Cas9 protein comprises a N611 mutation (e.g., a N611A mutation) or an analogous substitution to the amino acid corresponding to said position. In some embodiments, a catalytically inactive Cas9 protein, e.g., dCas9, comprises a D16 mutation (e.g., D16A), a D587 mutation (e.g., D587A), a H588 mutation (e.g., H588A), and a N611 mutation (e.g., N611A) or analogous substitutions to the amino acids corresponding to said positions.

In some embodiments, a DNA-binding domain or endonuclease domain may comprise a Cas molecule comprising or linked (e.g., covalently) to a gRNA (e.g., a template nucleic acid, e.g., template RNA, comprising a gRNA).

In some embodiments, an endonuclease domain or DNA binding domain comprises a Streptococcus pyogenes Cas9 (SpCas9) or a functional fragment or variant thereof. In some embodiments, the endonuclease domain or DNA binding domain comprises a modified SpCas9. In embodiments, the modified SpCas9 comprises a modification that alters protospacer-adjacent motif (PAM) specificity. In embodiments, the PAM has specificity for the nucleic acid sequence 5′-NGT-3′. In embodiments, the modified SpCas9 comprises one or more amino acid substitutions, e.g., at one or more of positions L1111, D1135, G1218, E1219, A1322, of R1335, e.g., selected from L1111R, D1135V, G1218R, E1219F, A1322R, R1335V. In embodiments, the modified SpCas9 comprises the amino acid substitution T1337R and one or more additional amino acid substitutions, e.g., selected from L1111, D1135L, S1136R, G1218S, E1219V, D1332A, D1332S, D1332T, D1332V, D1332L, D1332K, D1332R, R1335Q, T1337, T1337L, T1337Q, T1337I, T1337V, T1337F, T1337S, T1337N, T1337K, T1337H, T1337Q, and T1337M, or corresponding amino acid substitutions thereto. In embodiments, the modified SpCas9 comprises: (i) one or more amino acid substitutions selected from D1135L, S1136R, G1218S, E1219V, A1322R, R1335Q, and T1337; and (ii) one or more amino acid substitutions selected from L1111R, G1218R, E1219F, D1332A, D1332S, D1332T, D1332V, D1332L, D1332K, D1332R, T1337L, T1337I, T1337V, T1337F, T1337S, T1337N, T1337K, T1337R, T1337H, T1337Q, and T1337M, or corresponding amino acid substitutions thereto.

In some embodiments, the endonuclease domain or DNA binding domain comprises a Cas domain, e.g., a Cas9 domain. In embodiments, the endonuclease domain or DNA binding domain comprises a nuclease-active Cas domain, a Cas nickase (nCas) domain, or a nuclease-inactive Cas (dCas) domain. In embodiments, the endonuclease domain or DNA binding domain comprises a nuclease-active Cas9 domain, a Cas9 nickase (nCas9) domain, or a nuclease-inactive Cas9 (dCas9) domain. In some embodiments, the endonuclease domain or DNA binding domain comprises a Cas9 domain of Cas9 (e.g., dCas9 and nCas9), Cas12a/Cpf1, Cas12b/C2c1, Cas12c/C2c3, Cas12d/CasY, Cas12e/CasX, Cas12g, Cas12h, or Cas12i. In some embodiments, the endonuclease domain or DNA binding domain comprises a Cas9 (e.g., dCas9 and nCas9), Cas12a/Cpf1, Cas12b/C2c1, Cas12c/C2c3, Cas12d/CasY, Cas12e/CasX, Cas12g, Cas12h, or Cas12i. In some embodiments, the endonuclease domain or DNA binding domain comprises an S. pyogenes or an S. thermophilus Cas9, or a functional fragment thereof. In some embodiments, the endonuclease domain or DNA binding domain comprises a Cas9 sequence, e.g., as described in Chylinski, Rhun, and Charpentier (2013) RNA Biology 10:5, 726-737; incorporated herein by reference. In some embodiments, the endonuclease domain or DNA binding domain comprises the HNH nuclease subdomain and/or the RuvC1 subdomain of a Cas, e.g., Cas9, e.g., as described herein, or a variant thereof. In some embodiments, the endonuclease domain or DNA binding domain comprises Cas12a/Cpf1, Cas12b/C2c1, Cas12c/C2c3, Cas12d/CasY, Cas12e/CasX, Cas12g, Cas12h, or Cas12i. In some embodiments, the endonuclease domain or DNA binding domain comprises a Cas polypeptide (e.g., enzyme), or a functional fragment thereof. In embodiments, the Cas polypeptide (e.g., enzyme) is selected from Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (e.g., Csn1 or Csx12), Cas10, Cas10d, Cas12a/Cpf1, Cas12b/C2c1, Cas12c/C2c3, Cas12d/CasY, Cas12e/CasX, Cas12g, Cas12h, Cas12i, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, Cpf1, Cas12b/C2c1, Cas12c/C2c3, Cas12b/C2c1, Cas12c/C2c3, SpCas9 (K855A), eSpCas9 (1.1), SpCas9-HF1, hyper accurate Cas9 variant (HypaCas9), homologues thereof, modified or engineered versions thereof, and/or functional fragments thereof. In embodiments, the Cas9 comprises one or more substitutions, e.g., selected from H840A, D10A, P475A, W476A, N477A, D1125A, W1126A, and D1127A. In embodiments, the Cas9 comprises one or more mutations at positions selected from: D10, G12, G17, E762, H840, N854, N863, H982, H983, A984, D986, and/or A987, e.g., one or more substitutions selected from D10A, G12A, G17A, E762A, H840A, N854A, N863A, H982A, H983A, A984A, and/or D986A. In some embodiments, the endonuclease domain or DNA binding domain comprises a Cas (e.g., Cas9) sequence from Corynebacterium ulcerans, Corynebacterium diphtheria, Spiroplasma syrphidicola, Prevotella intermedia, Spiroplasma taiwanense, Streptococcus iniae, Belliella baltica, Psychroflexus torquis, Streptococcus thermophilus, Listeria innocua, Campylobacter jejuni, Neisseria meningitidis, Streptococcus pyogenes, or Staphylococcus aureus, or a fragment or variant thereof.

In some embodiments, the endonuclease domain or DNA binding domain comprises a Cpf1 domain, e.g., comprising one or more substitutions, e.g., at position D917, E1006A, D1255 or any combination thereof, e.g., selected from D917A, E1006A, D1255A, D917A/E1006A, D917A/D1255A, E1006A/D1255A, and D917A/E1006A/D1255A.

In some embodiments, the endonuclease domain or DNA binding domain comprises spCas9, spCas9-VRQR, spCas9-VRER, xCas9 (sp), saCas9, saCas9-KKH, spCas9-MQKSER, spCas9-LRKIQK, or spCas9-LRVSQL.

In some embodiments, a heterologous gene modifying polypeptide has an endonuclease domain comprising a Cas9 nickase, e.g., Cas9 H840A. In embodiments, the Cas9 H840A has the following amino acid sequence:

Cas9 nickase (H840A):
(SEQ ID NO: 11,001)
DKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDAIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD

In some embodiments, a heterologous gene modifying polypeptide comprises a dCas9 sequence comprising a D10A and/or H840A mutation, e.g., the following sequence:

(SEQ ID NO: 5007)
SMDKKYSIGLAIGTNSVGWAVITDDYKVPSKKFKVLGNTDRHSIKKNLIG
ALLFDSGETAEATRLKRTARRRYTRRKNRICYLQEIFSNEMAKVDDSFFH
RLEESFLVEEDKKHERHPIFGNIVDEVAYHEKYPTIYHLRKKLVDSTDKA
DLRLIYLALAHMIKFRGHFLIEGDLNPDNSDVDKLFIQLVQTYNQLFEEN
PINASGVDAKAILSARLSKSRRLENLIAQLPGEKKNGLFGNLIALSLGLT
PNFKSNFDLAEDAKLQLSKDTYDDDLDNLLAQIGDQYADLFLAAKNLSDA
ILLSDILRVNTEITKAPLSASMIKRYDEHHQDLTLLKALVRQQLPEKYKE
IFFDQSKNGYAGYIDGGASQEEFYKFIKPILEKMDGTEELLVKLNREDLL
RKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFD
KNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLK
IIKDKDFLDNEENEDILEDIVLTLTLFEDREMIEERLKTYAHLFDDKVMK
QLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEH
PVENTQLQNEKLYLYYLQNGRDMYVDQELDINRLSDYDVDAIVPQSFLKD
DSIDNKVLTRSDKNRGKSDNVPSEEVVKKMKNYWRQLLNAKLITQRKFDN
LTKAERGGLSELDKAGFIKRQLVETRQITKHVAQILDSRMNTKYDENDKL
IREVKVITLKSKLVSDFRKDFQFYKVREINNYHHAHDAYLNAVVGTALIK
KYPKLESEFVYGDYKVYDVRKMIAKSEQEIGKATAKYFFYSNIMNFFKTE
ITLANGEIRKRPLIETNGETGEIVWDKGRDFATVRKVLSMPQVNIVKKTE
VQTGGFSKESILPKRNSDKLIARKKDWDPKKYGGFDSPTVAYSVLVVAKV
EKGKSKKLKSVKELLGITIMERSSFEKNPIDFLEAKGYKEVKKDLIIKLP
KYSLFELENGRKRMLASAGELQKGNELALPSKYVNFLYLASHYEKLKGSP
EDNEQKQLFVEQHKHYLDEIIEQISEFSKRVILADANLDKVLSAYNKHRD
KPIREQAENIIHLFTLTNLGAPAAFKYFDTTIDRKRYTSTKEVLDATLIH
QSITGLYETRIDLSQLGGD

### TAL Effectors and Zinc Finger Nucleases

In some embodiments, an endonuclease domain or DNA-binding domain comprises a TAL effector molecule. A TAL effector molecule, e.g., a TAL effector molecule that specifically binds a DNA sequence, typically comprises a plurality of TAL effector domains or fragments thereof, and optionally one or more additional portions of naturally occurring TAL effectors (e.g., N- and/or C-terminal of the plurality of TAL effector domains). Many TAL effectors are known to those of skill in the art and are commercially available, e.g., from Thermo Fisher Scientific.

Naturally occurring TALEs are natural effector proteins secreted by numerous species of bacterial pathogens including the plant pathogen Xanthomonas which modulates gene expression in host plants and facilitates bacterial colonization and survival. The specific binding of TAL effectors is based on a central repeat domain of tandemly arranged nearly identical repeats of typically 33 or 34 amino acids (the repeat-variable di-residues, RVD domain).

Members of the TAL effectors family differ mainly in the number and order of their repeats. The number of repeats typically ranges from 1.5 to 33.5 repeats and the C-terminal repeat is usually shorter in length (e.g., about 20 amino acids) and is generally referred to as a “half-repeat.” Each repeat of the TAL effector generally features a one-repeat-to-one-base-pair correlation with different repeat types exhibiting different base-pair specificity (one repeat recognizes one base-pair on the target gene sequence). Generally, the smaller the number of repeats, the weaker the protein-DNA interactions. A number of 6.5 repeats has been shown to be sufficient to activate transcription of a reporter gene (Scholze et al., 2010).

Repeat to repeat variations occur predominantly at amino acid positions 12 and 13, which have therefore been termed “hypervariable” and which are responsible for the specificity of the interaction with the target DNA promoter sequence, as shown in Table 9 listing exemplary repeat variable diresidues (RVD) and their correspondence to nucleic acid base targets.

TABLE 9
RVDs and Nucleic Acid Base Specificity

| Target | Possible RVD Amino Acid Combinations | | | | | | | | | | | | |
|---|---|---|---|---|---|---|---|---|---|---|---|---|---|
| A | NI | NN | CI | HI | KI | | | | | | | | |
| G | NN | GN | SN | VN | LN | DN | QN | EN | HN | RH | NK | AN | FN |
| C | HD | RD | KD | ND | AD | | | | | | | | |
| T | NG | HG | VG | IG | EG | MG | YG | AA | EP | VA | QG | KG | RG |

Accordingly, it is possible to modify the repeats of a TAL effector to target specific DNA sequences. Further studies have shown that the RVD NK can target G. Target sites of TAL effectors also tend to include a T flanking the 5′ base targeted by the first repeat, but the exact mechanism of this recognition is not known. More than 113 TAL effector sequences are known to date. Non-limiting examples of TAL effectors from Xanthomonas include, Hax2, Hax3, Hax4, AvrXa7, AvrXa10 and AvrBs3.

Accordingly, the TAL effector domain of a TAL effector molecule described herein may be derived from a TAL effector from any bacterial species (e.g., Xanthomonas species such as the African strain of Xanthomonas oryzae pv. Oryzae (Yu et al. 2011), Xanthomonas campestris pv. raphani strain 756C and Xanthomonas oryzae pv. oryzicola strain BLS256 (Bogdanove et al. 2011). In some embodiments, the TAL effector domain comprises an RVD domain as well as flanking sequence(s) (sequences on the N-terminal and/or C-terminal side of the RVD domain) also from the naturally occurring TAL effector. It may comprise more or fewer repeats than the RVD of the naturally occurring TAL effector. The TAL effector molecule can be designed to target a given DNA sequence based on the above code and others known in the art. The number of TAL effector domains (e.g., repeats (monomers or modules)) and their specific sequence can be selected based on the desired DNA target sequence. For example, TAL effector domains, e.g., repeats, may be removed or added in order to suit a specific target sequence. In an embodiment, the TAL effector molecule of the present invention comprises between 6.5 and 33.5 TAL effector domains, e.g., repeats. In an embodiment, TAL effector molecule of the present invention comprises between 8 and 33.5 TAL effector domains, e.g., repeats, e.g., between 10 and 25 TAL effector domains, e.g., repeats, e.g., between 10 and 14 TAL effector domains, e.g., repeats.

In some embodiments, the TAL effector molecule comprises TAL effector domains that correspond to a perfect match to the DNA target sequence. In some embodiments, a mismatch between a repeat and a target base-pair on the DNA target sequence is permitted as along as it allows for the function of the polypeptide comprising the TAL effector molecule. In general, TALE binding is inversely correlated with the number of mismatches. In some embodiments, the TAL effector molecule of a polypeptide of the present invention comprises no more than 7 mismatches, 6 mismatches, 5 mismatches, 4 mismatches, 3 mismatches, 2 mismatches, or 1 mismatch, and optionally no mismatch, with the target DNA sequence. Without wishing to be bound by theory, in general the smaller the number of TAL effector domains in the TAL effector molecule, the smaller the number of mismatches will be tolerated and still allow for the function of the polypeptide comprising the TAL effector molecule. The binding affinity is thought to depend on the sum of matching repeat-DNA combinations. For example, TAL effector molecules having 25 TAL effector domains or more may be able to tolerate up to 7 mismatches.

In addition to the TAL effector domains, the TAL effector molecule of the present invention may comprise additional sequences derived from a naturally occurring TAL effector. The length of the C-terminal and/or N-terminal sequence(s) included on each side of the TAL effector domain portion of the TAL effector molecule can vary and be selected by one skilled in the art, for example based on the studies of Zhang et al. (2011). Zhang et al., have characterized a number of C-terminal and N-terminal truncation mutants in Hax3 derived TAL-effector based proteins and have identified key elements, which contribute to optimal binding to the target sequence and thus activation of transcription. Generally, it was found that transcriptional activity is inversely correlated with the length of N-terminus. Regarding the C-terminus, an important element for DNA binding residues within the first 68 amino acids of the Hax 3 sequence was identified. Accordingly, in some embodiments, the first 68 amino acids on the C-terminal side of the TAL effector domains of the naturally occurring TAL effector is included in the TAL effector molecule. Accordingly, in an embodiment, a TAL effector molecule comprises 1) one or more TAL effector domains derived from a naturally occurring TAL effector; 2) at least 70, 80, 90, 100, 110, 120, 130, 140, 150, 170, 180, 190, 200, 220, 230, 240, 250, 260, 270, 280 or more amino acids from the naturally occurring TAL effector on the N-terminal side of the TAL effector domains; and/or 3) at least 68, 80, 90, 100, 110, 120, 130, 140, 150, 170, 180, 190, 200, 220, 230, 240, 250, 260 or more amino acids from the naturally occurring TAL effector on the C-terminal side of the TAL effector domains.

In some embodiments, an endonuclease domain or DNA-binding domain is or comprises a Zn finger molecule. A Zn finger molecule comprises a Zn finger protein, e.g., a naturally occurring Zn finger protein or engineered Zn finger protein, or fragment thereof. Many Zn finger proteins are known to those of skill in the art and are commercially available, e.g., from Sigma-Aldrich.

In some embodiments, a Zn finger molecule comprises a non-naturally occurring Zn finger protein that is engineered to bind to a target DNA sequence of choice. See, for example, Beerli, et al. (2002) Nature Biotechnol. 20:135-141; Pabo, et al. (2001) Ann. Rev. Biochem. 70:313-340; Isalan, et al. (2001) Nature Biotechnol. 19:656-660; Segal, et al. (2001) Curr. Opin. Biotechnol. 12:632-637; Choo, et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

An engineered Zn finger protein may have a novel binding specificity, compared to a naturally-occurring Zn finger protein. Engineering methods include, but are not limited to, rational design and various types of selection. Rational design includes, for example, using databases comprising triplet (or quadruplet) nucleotide sequences and individual Zn finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as International Patent Publication Nos. WO 98/37186; WO 98/53057; WO 00/27878; and WO 01/88197 and GB 2,338,237. In addition, enhancement of binding specificity for zinc finger proteins has been described, for example, in International Patent Publication No. WO 02/077227.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein. In addition, enhancement of binding specificity for zinc finger binding domains has been described, for example, in co-owned International Patent Publication No. WO 02/077227.

Zn finger proteins and methods for design and construction of fusion proteins (and polynucleotides encoding same) are known to those of skill in the art and described in detail in U.S. Pat. Nos. 6,140,0815; 789,538; 6,453,242; 6,534,261; 5,925,523; 6,007,988; 6,013,453; and 6,200,759; International Patent Publication Nos. WO 95/19431; WO 96/06166; WO 98/53057; WO 98/54311; WO 00/27878; WO 01/60970; WO 01/88197; WO 02/099084; WO 98/53058; WO 98/53059; WO 98/53060; WO 02/016536; and WO 03/016496.

In addition, as disclosed in these and other references, Zn finger proteins and/or multi-fingered Zn finger proteins may be linked together, e.g., as a fusion protein, using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The Zn finger molecules described herein may include any combination of suitable linkers between the individual zinc finger proteins and/or multi-fingered Zn finger proteins of the Zn finger molecule.

In certain embodiments, the DNA-binding domain or endonuclease domain comprises a Zn finger molecule comprising an engineered zinc finger protein that binds (in a sequence-specific manner) to a target DNA sequence. In some embodiments, the Zn finger molecule comprises one Zn finger protein or fragment thereof. In other embodiments, the Zn finger molecule comprises a plurality of Zn finger proteins (or fragments thereof), e.g., 2, 3, 4, 5, 6 or more Zn finger proteins (and optionally no more than 12, 11, 10, 9, 8, 7, 6, 5, 4, 3, or 2 Zn finger proteins). In some embodiments, the Zn finger molecule comprises at least three Zn finger proteins. In some embodiments, the Zn finger molecule comprises four, five, or six fingers. In some embodiments, the Zn finger molecule comprises 8, 9, 10, 11, or 12 fingers. In some embodiments, a Zn finger molecule comprising three Zn finger proteins recognizes a target DNA sequence comprising 9 or 10 nucleotides. In some embodiments, a Zn finger molecule comprising four Zn finger proteins recognizes a target DNA sequence comprising 12 to 14 nucleotides. In some embodiments, a Zn finger molecule comprising six Zn finger proteins recognizes a target DNA sequence comprising 18 to 21 nucleotides.

In some embodiments, a Zn finger molecule comprises a two-handed Zn finger protein. Two handed zinc finger proteins are those proteins in which two clusters of zinc finger proteins are separated by intervening amino acids so that the two zinc finger domains bind to two discontinuous target DNA sequences. An example of a two handed type of zinc finger binding protein is SIP1, where a cluster of four zinc finger proteins is located at the amino terminus of the protein and a cluster of three Zn finger proteins is located at the carboxyl terminus (see Remade, et al. (1999) EMBO Journal 18 (18): 5073-5084). Each cluster of zinc fingers in these proteins is able to bind to a unique target sequence and the spacing between the two target sequences can comprise many nucleotides.

### Linkers

In some embodiments, a heterologous gene modifying polypeptide may comprise a linker, e.g., a peptide linker, e.g., a linker as described in Table 10. In some embodiments, a heterologous gene modifying polypeptide comprises, in an N-terminal to C-terminal direction, a Cas domain (e.g., a Cas domain of Table 8), a linker of Table 10 (or a sequence having at least 70%, 80%, 85%, 90%, 95%, or 99% identity thereto), and an RT domain (e.g., an RT domain of Table 6). In some embodiments, a heterologous gene modifying polypeptide comprises a flexible linker between the endonuclease and the RT domain, e.g., a linker comprising the amino acid sequence SGGSSGGSSGSETPGTSESATPESSGGSSGGSS (SEQ ID NO: 11,002). In some embodiments, an RT domain of a heterologous gene modifying polypeptide may be located C-terminal to the endonuclease domain. In some embodiments, an RT domain of a heterologous gene modifying polypeptide may be located N-terminal to the endonuclease domain.

TABLE 10
Exemplary linker sequences

| | SEQ ID |
|---|---|
| Amino Acid Sequence | NO |
| GGS | |
| GGSGGS | 5102 |
| GGSGGSGGS | 5103 |
| GGSGGSGGSGGS | 5104 |
| GGSGGSGGSGGSGGS | 5105 |
| GGSGGSGGSGGSGGSGGS | 5106 |
| GGGGS | 5107 |
| GGGGSGGGGS | 5108 |
| GGGGSGGGGSGGGGS | 5109 |
| GGGGSGGGGSGGGGSGGGGS | 5110 |
| GGGGSGGGGSGGGGSGGGGSGGGGS | 5111 |
| GGGGSGGGGSGGGGSGGGGSGGGGSGGGGS | 5112 |
| GGG | |
| GGGG | 5114 |
| GGGGG | 5115 |
| GGGGGG | 5116 |
| GGGGGGG | 5117 |
| GGGGGGGG | 5118 |
| GSS | |
| GSSGSS | 5120 |
| GSSGSSGSS | 5121 |
| GSSGSSGSSGSS | 5122 |
| GSSGSSGSSGSSGSS | 5123 |
| GSSGSSGSSGSSGSSGSS | 5124 |
| EAAAK | 5125 |
| EAAAKEAAAK | 5126 |
| EAAAKEAAAKEAAAK | 5127 |
| EAAAKEAAAKEAAAKEAAAK | 5128 |
| EAAAKEAAAKEAAAKEAAAKEAAAK | 5129 |
| EAAAKEAAAKEAAAKEAAAKEAAAKEAAAK | 5130 |
| PAP | |
| PAPAP | 5132 |
| PAPAPAP | 5133 |
| PAPAPAPAP | 5134 |
| PAPAPAPAPAP | 5135 |
| PAPAPAPAPAPAP | 5136 |
| GGSGGG | 5137 |
| GGGGGS | 5138 |
| GGSGSS | 5139 |
| GSSGGS | 5140 |
| GGSEAAAK | 5141 |
| EAAAKGGS | 5142 |
| GGSPAP | 5143 |
| PAPGGS | 5144 |
| GGGGSS | 5145 |
| GSSGGG | 5146 |
| GGGEAAAK | 5147 |
| EAAAKGGG | 5148 |
| GGGPAP | 5149 |
| PAPGGG | 5150 |
| GSSEAAAK | 5151 |
| EAAAKGSS | 5152 |
| GSSPAP | 5153 |
| PAPGSS | 5154 |
| EAAAKPAP | 5155 |
| PAPEAAAK | 5156 |
| GGSGGGGSS | 5157 |
| GGSGSSGGG | 5158 |
| GGGGGSGSS | 5159 |
| GGGGSSGGS | 5160 |
| GSSGGSGGG | 5161 |
| GSSGGGGGS | 5162 |
| GGSGGGEAAAK | 5163 |
| GGSEAAAKGGG | 5164 |
| GGGGGSEAAAK | 5165 |
| GGGEAAAKGGS | 5166 |
| EAAAKGGSGGG | 5167 |
| EAAAKGGGGGS | 5168 |
| GGSGGGPAP | 5169 |
| GGSPAPGGG | 5170 |
| GGGGGSPAP | 5171 |
| GGGPAPGGS | 5172 |
| PAPGGSGGG | 5173 |
| PAPGGGGGS | 5174 |
| GGSGSSEAAAK | 5175 |
| GGSEAAAKGSS | 5176 |
| GSSGGSEAAAK | 5177 |
| GSSEAAAKGGS | 5178 |
| EAAAKGGSGSS | 5179 |
| EAAAKGSSGGS | 5180 |
| GGSGSSPAP | 5181 |
| GGSPAPGSS | 5182 |
| GSSGGSPAP | 5183 |
| GSSPAPGGS | 5184 |
| PAPGGSGSS | 5185 |
| PAPGSSGGS | 5186 |
| GGSEAAAKPAP | 5187 |
| GGSPAPEAAAK | 5188 |
| EAAAKGGSPAP | 5189 |
| EAAAKPAPGGS | 5190 |
| PAPGGSEAAAK | 5191 |
| PAPEAAAKGGS | 5192 |
| GGGGSSEAAAK | 5193 |
| GGGEAAAKGSS | 5194 |
| GSSGGGEAAAK | 5195 |
| GSSEAAAKGGG | 5196 |
| EAAAKGGGGSS | 5197 |
| EAAAKGSSGGG | 5198 |
| GGGGSSPAP | 5199 |
| GGGPAPGSS | 5200 |
| GSSGGGPAP | 5201 |
| GSSPAPGGG | 5202 |
| PAPGGGGSS | 5203 |
| PAPGSSGGG | 5204 |
| GGGEAAAKPAP | 5205 |
| GGGPAPEAAAK | 5206 |
| EAAAKGGGPAP | 5207 |
| EAAAKPAPGGG | 5208 |
| PAPGGGEAAAK | 5209 |
| PAPEAAAKGGG | 5210 |
| GSSEAAAKPAP | 5211 |
| GSSPAPEAAAK | 5212 |
| EAAAKGSSPAP | 5213 |
| EAAAKPAPGSS | 5214 |
| PAPGSSEAAAK | 5215 |
| PAPEAAAKGSS | 5216 |
| AEAAAKEAAAKEAAAKEAAAKALEAEAAAKEAA | 5217 |
| AKEAAAKEAAAKA | |
| GGGGSEAAAKGGGGS | 5218 |
| EAAAKGGGGSEAAAK | 5219 |
| SGSETPGTSESATPES | 5220 |
| GSAGSAAGSGEF | 5221 |
| SGGSSGGSSGSETPGTSESATPESSGGSSGGSS | 5222 |

In some embodiments, a linker of a heterologous gene modifying polypeptide comprises a motif chosen from: (SGGS)ₙ (SEQ ID NO: 5025), (GGGS)ₙ (SEQ ID NO: 5026), (GGGGS)ₙ (SEQ ID NO: 5027), (G)ₙ, (EAAAK)ₙ (SEQ ID NO: 5028), (GGS)ₙ, or (XP)ₙ.

### Heterologous Gene Modifying Polypeptide Selection by Pooled Screening

Candidate heterologous gene modifying polypeptides may be screened to evaluate a candidate's gene editing ability. For example, an RNA heterologous gene modifying system designed for the targeted editing of a coding sequence in the human genome may be used. In certain embodiments, such a heterologous gene modifying system may be used in conjunction with a pooled screening approach.

For example, a library of heterologous gene modifying polypeptide candidates and a template guide RNA (tgRNA) may be introduced into mammalian cells to test the candidates' gene editing abilities by a pooled screening approach. In specific embodiments, a library of heterologous gene modifying polypeptide candidates is introduced into mammalian cells followed by introduction of the tgRNA into the cells.

Representative, non-limiting examples of mammalian cells that may be used in screening include HEK293T cells, U2OS cells, HeLa cells, HepG2 cells, Huh7 cells, K562 cells, or iPS cells.

A heterologous gene modifying polypeptide candidate may comprise 1) a Cas-nuclease, for example a wild-type Cas nuclease, e.g., a wild-type Cas9 nuclease, a mutant Cas nuclease, e.g., a Cas nickase, for example, a Cas9 nickase such as a Cas9 N863A nickase, or a Cas nuclease selected from Table 7 or 8, 2) a peptide linker, e.g., a sequence from Table D or 10, that may exhibit varying degrees of length, flexibility, hydrophobicity, and/or secondary structure; and 3) a reverse transcriptase (RT), e.g. an RT domain from Table D or 6. A heterologous gene modifying polypeptide candidate library comprises: a plurality of different heterologous gene modifying polypeptide candidates that differ from each other with respect to one, two or all three of the Cas nuclease, peptide linker or RT domain components, or a plurality of nucleic acid expression vectors that encode such heterologous gene modifying polypeptide candidates.

For screening of heterologous gene modifying polypeptide candidates, a two-component system may be used that comprises a heterologous gene modifying polypeptide component and a tgRNA component. A gene modifying component may comprise, for example, an expression vector, e.g., an expression plasmid or lentiviral vector, that encodes a heterologous gene modifying polypeptide candidate, for example, comprises a human codon-optimized nucleic acid that encodes a heterologous gene modifying polypeptide candidate, e.g., a Cas-linker-RT fusion as described above. In a particular embodiment, a lentiviral cassette is utilized that comprises: (i) a promoter for expression in mammalian cells, e.g., a CMV promoter; (ii) a gene modifying library candidate, e.g. a Cas-linker-RT fusion comprising a Cas nuclease of Table CC, a peptide linker of Table AA and an RT of Table BB, for example a Cas-linker-RT fusion as in Table D; (iii) a self-cleaving polypeptide, e.g., a T2A peptide; (iv) a marker enabling selection in mammalian cells, e.g., a puromycin resistance gene; and (v) a termination signal, e.g., a poly A tail.

The tgRNA component may comprise a tgRNA or expression vector, e.g., an expression plasmid, that produces the tgRNA, for example, utilizes a U6 promoter to drive expression of the tgRNA, wherein the tgRNA is a non-coding RNA sequence that is recognized by Cas and localizes it to the genomic locus of interest, and that also templates reverse transcription of the desired edit into the genome by the RT domain.

To prepare a pool of cells expressing heterologous gene modifying polypeptide library candidates, mammalian cells, e.g., HEK293T or U2OS cells, may be transduced with pooled heterologous gene modifying polypeptide candidate expression vector preparations, e.g., lentiviral preparations, of the gene modifying candidate polypeptide library. In a particular embodiment, lentiviral plasmids are utilized, and HEK293 Lenti-X cells are seeded in 15 cm plates (~12×10⁶ cells) prior to lentiviral plasmid transfection. In such an embodiment, lentiviral plasmid transfection may be performed using the Lentiviral Packaging Mix (Biosettia) and transfection of the plasmid DNA for the gene modifying candidate library is performed the following day using Lipofectamine 2000 and Opti-MEM media according to the manufacturer's protocol. In such an embodiment, extracellular DNA may be removed by a full media change the next day and virus-containing media may be harvested 48 hours after. Lentiviral media may be concentrated using Lenti-X Concentrator (TaKaRa Biosciences) and 5 mL lentiviral aliquots may be made and stored at −80° C. Lentiviral titering is performed by enumerating colony forming units post-selection, e.g., post Puromycin selection.

For monitoring gene editing of a target DNA, mammalian cells, e.g., HEK293T or U20S cells, carrying a target DNA may be utilized. In other embodiments for monitoring gene editing of a target DNA, mammalian cells, e.g., HEK293T or U2OS cells, carrying a target DNA genomic landing pad may be utilized. In particular embodiments, the target DNA genomic landing pad may comprise a gene to be edited for treatment of a disease or disorder of interest. In other particular embodiments, the target DNA is a gene sequence that expresses a protein that exhibits detectable characteristics that may be monitored to determine whether gene editing has occurred. For example, in certain embodiments, a blue fluorescence protein (BFP)− or green fluorescence protein (GFP)-expressing genomic landing pad is utilized. In certain embodiments, mammalian cells, e.g., HEK293T or U2OS cells, comprising a target DNA, e.g., a target DNA genomic landing pad, are seeded in culture plates at 500×-3000× cells per gene modifying library candidate and transduced at a 0.2-0.3 multiplicity of infection (MOI) to minimize multiple infections per cell. Puromycin (2.5 μg/mL) may be added 48 hours post infection to allow for selection of infected cells. In such an embodiment, cells may be kept under puromycin selection for at least 7 days and then scaled up for tgRNA introduction, e.g., tgRNA electroporation.

To ascertain whether gene editing occurs, mammalian cells containing a target DNA to be edited may be infected with heterologous gene modifying polypeptide library candidates then transfected with tgRNA designed for use in editing of the target DNA. Subsequently, the cells may be analyzed to determine whether editing of the target locus has occurred according to the designed outcome, or whether no editing or imperfect editing has occurred, e.g., by using cell sorting and sequence analysis.

In a particular embodiment, to ascertain whether genome editing occurs, BFP− or GFP− expressing mammalian cells, e.g., HEK293T or U2OS cells, may be infected with gene modifying library candidates and then transfected or electroporated with tgRNA plasmid or RNA, e.g., by electroporation of 250,000 cells/well with 200 ng of a tgRNA plasmid designed to convert BFP-to-GFP or GFP-to-BFP, at a cell count ensuring>250×-1000× coverage per library candidate. In such an embodiment, the genome-editing capacity of the various constructs in this assay may be assessed by sorting the cells by Fluorescence-Activated Cell Sorting (FACS) for expression of the color-converted fluorescent protein (FP) at 4-10 days post-electroporation. Cells are sorted and harvested as distinct populations of unedited cells (exhibiting original florescence protein signal), edited cells (exhibiting converted fluorescence protein signal), and imperfect edit (exhibiting no florescence protein signal) cells. A sample of unsorted cells may also be harvested as the input population to determine candidate enrichment during analysis.

To determine which gene modifying library candidates exhibit genome-editing capacity in an assay, genomic DNA (gDNA) is harvested from the sorted cell populations, and analyzed by sequencing the gene modifying library candidates in each population. Briefly, gene modifying candidates may be amplified from the genome using primers specific to the heterologous gene modifying polypeptide expression vector, e.g., the lentiviral cassette, amplified in a second round of PCR to dilute genomic DNA, and then sequenced, for example, sequenced by a next-generation sequencing platform. After quality control of sequencing reads, reads of at least about 1500 nucleotides and generally no more than about 3200 nucleotides are mapped to the heterologous gene modifying polypeptide library sequences and those containing a minimum of about an 80% match to a library sequence are considered to be successfully aligned to a given candidate for purposes of this pooled screen. In order to identify candidates capable of performing gene editing in the assay, e.g., the BFP-to-GFP or GFP-to-BFP edit, the read count of each library candidate in the edited population is compared to its read count in the initial, unsorted population.

For purposes of pooled screening, gene modifying candidates with genome-editing capacity are identified based on enrichment in the edited (converted FP) population relative to unsorted (input) cells. In some embodiments, an enrichment of at least 1.0, 1.5, 2.0, 2.5, 3.0, 4.0, 5.0, 6.0, 7.0, 8.0, 9.0, 10, 15, 20, 25, 30, 40, 50, 60, 70, 80, 90, or at least 100-fold over the input indicates potentially useful gene editing activity, e.g., at least 2-fold enrichment. In some embodiments, the enrichment is converted to a log-value by taking the log base 2 of the enrichment ratio. In some embodiments, a log 2 enrichment score of at least 0, 1, 2, 3, 4, 5, 5.5, 6.0, 6.2, 6.3, 6.4, 6.5, or at least 6.6 indicates potentially useful gene editing activity, e.g., a log 2 enrichment score of at least 1.0. In particular embodiments, enrichment values observed for gene modifying candidates may be compared to enrichment values observed under similar conditions utilizing a reference, e.g., Element ID No: 17380.

In some embodiments, multiple tgRNAs may be used to screen the gene modifying candidate library. In particular embodiments, a plurality of tgRNAs may be utilized to optimize template/Cas-linker-RT fusion pairs, e.g., for gene editing of particular target genes, for example, gene targets for the treatment of disease. In specific embodiments, a pooled approach to screening gene modifying candidates may be performed using a multiplicity of different tgRNAs in an arrayed format.

In some embodiments, multiple types of edits, e.g., insertions, substitutions, and/or deletions of different lengths, may be used to screen the gene modifying candidate library.

In some embodiments, multiple target sequences, e.g., different fluorescent proteins, may be used to screen the gene modifying candidate library. In some embodiments, multiple target sequences, e.g., different fluorescent proteins, may be used to screen the gene modifying candidate library. In some embodiments, multiple cell types, e.g., HEK293T or U2OS, may be used to screen the gene modifying candidate library. The person of ordinary skill in the art will appreciate that a given candidate may exhibit altered editing capacity or even the gain or loss of any observable or useful activity across different conditions, including tgRNA sequence (e.g., nucleotide modifications, PBS length, RT template length), target sequence, target location, type of edit, location of mutation relative to the first-strand nick of the heterologous gene modifying polypeptide, or cell type. Thus, in some embodiments, gene modifying library candidates are screened across multiple parameters, e.g., with at least two distinct tgRNAs in at least two cell types, and gene editing activity is identified by enrichment in any single condition. In other embodiments, a candidate with more robust activity across different tgRNA and cell types is identified by enrichment in at least two conditions, e.g., in all conditions screened. For clarity, candidates found to exhibit little to no enrichment under any given condition are not assumed to be inactive across all conditions and may be screened with different parameters or reconfigured at the polypeptide level, e.g., by swapping, shuffling, or evolving domains (e.g., RT domain), linkers, or other signals (e.g., NLS).

### Sequences of Exemplary Cas9-Linker-RT Fusions

In some embodiments, a heterologous gene modifying polypeptide comprises a linker sequence and an RT sequence. In some embodiments, a heterologous gene modifying polypeptide comprises a linker sequence as listed in Table D, or an amino acid sequence having at least 75%, 80%, 85%, 90%, 95%, 96%, 97%, 98%, or 99% identity thereto. In some embodiments, a heterologous gene modifying polypeptide comprises the amino acid sequence of an RT domain as listed in Table D, or an amino acid sequence having at least 75%, 80%, 85%, 90%, 95%, 96%, 97%, 98%, or 99% identity thereto. In some embodiments, a heterologous gene modifying polypeptide comprises a linker sequence as listed in Table D, or an amino acid sequence having at least 75%, 80%, 85%, 90%, 95%, 96%, 97%, 98%, or 99% identity thereto; and the amino acid sequence of an RT domain as listed in Table D, or an amino acid sequence having at least 75%, 80%, 85%, 90%, 95%, 96%, 97%, 98%, or 99% identity thereto. In some embodiments, a heterologous gene modifying polypeptide comprises: (i) a linker sequence as listed in a row of Table D, or an amino acid sequence having at least 75%, 80%, 85%, 90%, 95%, 96%, 97%, 98%, or 99% identity thereto; and (ii) the amino acid sequence of an RT domain as listed in the same row of Table D, or an amino acid sequence having at least 75%, 80%, 85%, 90%, 95%, 96%, 97%, 98%, or 99% identity thereto.

### Exemplary Heterologous Gene Modifying Polypeptides

In some embodiments, a heterologous gene modifying polypeptide (e.g., a gene heterologous modifying polypeptide that is part of a system described herein) comprises an amino acid sequence of any one of SEQ ID NOs: 1-7743, or an amino acid sequence having at least 70%, 75%, 80%, 85%, 90%, 95%, or 99% identity thereto. In some embodiments, a heterologous gene modifying polypeptide comprises an amino acid sequence of any one of SEQ ID NOs: 1-7743, or an amino acid sequence having at least 80% identity thereto. In some embodiments, a heterologous gene modifying polypeptide comprises an amino acid sequence of any one of SEQ ID NOs: 1-7743, or an amino acid sequence having at least 90% identity thereto. In some embodiments, a heterologous gene modifying polypeptide comprises an amino acid sequence of any one of SEQ ID NOs: 1-7743, or an amino acid sequence having at least 95% identity thereto. In some embodiments, a heterologous gene modifying polypeptide comprises an amino acid sequence of any one of SEQ ID NOs: 1-7743, or an amino acid sequence having at least 99% identity thereto. In some embodiments, a heterologous gene modifying polypeptide comprises an amino acid sequence of any one of SEQ ID NOs: 1-7743. In some embodiments, a heterologous gene modifying polypeptide comprises an amino acid sequence of any one of SEQ ID NOs: 6001-7743, or an amino acid sequence having at least 70%, 75%, 80%, 85%, 90%, 95%, or 99% identity thereto. In some embodiments, a heterologous gene modifying polypeptide comprises an amino acid sequence of any one of SEQ ID NOs: 4501-4541, or an amino acid sequence having at least 70%, 75%, 80%, 85%, 90%, 95%, or 99% identity thereto.

In some embodiments, a heterologous gene modifying polypeptide comprises an amino acid sequence as listed in Table A1, or an amino acid sequence having at least 70%, 75%, 80%, 85%, 90%, 95%, or 99% identity thereto.

In some embodiments, a heterologous gene modifying polypeptide comprises an amino acid sequence as listed in Table T1, or an amino acid sequence having at least 70%, 75%, 80%, 85%, 90%, 95%, or 99% identity thereto. In some embodiments, a heterologous gene modifying polypeptide comprises a linker comprising a linker sequence as listed in Table T1, or an amino acid sequence having at least 70%, 75%, 80%, 85%, 90%, 95%, or 99% identity thereto. In some embodiments, a heterologous gene modifying polypeptide comprises an RT domain comprising an RT domain sequence as listed in Table T1, or an amino acid sequence having at least 70%, 75%, 80%, 85%, 90%, 95%, or 99% identity thereto. In some embodiments, a heterologous gene modifying polypeptide comprises: (i) a linker comprising a linker sequence as listed in a row of Table T1, or an amino acid sequence having at least 70%, 75%, 80%, 85%, 90%, 95%, or 99% identity thereto; and (ii) an RT domain comprising an RT domain sequence as listed in the same row of Table T1, or an amino acid sequence having at least 70%, 75%, 80%, 85%, 90%, 95%, or 99% identity thereto.

TABLE T1
Selection of exemplary heterologous gene modifying polypeptides

| SEQ ID NO: | | | |
|---|---|---|---|
| for Full | | SEQ ID | |
| Polypeptide | | NO: of | |
| Sequence | Linker Sequence | linker | RT name |
| 1372 | AEAAAKEAAAKEAAAKEAAAKALEAEAAAKE | 15,401 | AVIRE_P03360_3mutA |
| | AAAKEAAAKEAAAKA | | |
| 1197 | AEAAAKEAAAKEAAAKEAAAKALEAEAAAKE | 15,402 | FLV_P10273_3mutA |
| | AAAKEAAAKEAAAKA | | |
| 2784 | AEAAAKEAAAKEAAAKEAAAKALEAEAAAKE | 15,403 | MLVMS_P03355_3mutA_WS |
| | AAAKEAAAKEAAAKA | | |
| 647 | AEAAAKEAAAKEAAAKEAAAKALEAEAAAKE | 15,404 | SFV3L_P27401_2mutA |
| | AAAKEAAAKEAAAKA | | |

In some embodiments, a heterologous gene modifying polypeptide comprises an amino acid sequence as listed in Table T2, or an amino acid sequence having at least 70%, 75%, 80%, 85%, 90%, 95%, or 99% identity thereto. In some embodiments, a heterologous gene modifying polypeptide comprises a linker comprising a linker sequence as listed in Table T2, or an amino acid sequence having at least 70%, 75%, 80%, 85%, 90%, 95%, or 99% identity thereto. In some embodiments, a heterologous gene modifying polypeptide comprises an RT domain comprising an RT domain sequence as listed in Table T2, or an amino acid sequence having at least 70%, 75%, 80%, 85%, 90%, 95%, or 99% identity thereto. In some embodiments, a heterologous gene modifying polypeptide comprises: (i) a linker comprising a linker sequence as listed in a row of Table T2, or an amino acid sequence having at least 70%, 75%, 80%, 85%, 90%, 95%, or 99% identity thereto; and (ii) an RT domain comprising an RT domain sequence as listed in the same row of Table T2, or an amino acid sequence having at least 70%, 75%, 80%, 85%, 90%, 95%, or 99% identity thereto.

TABLE T2
Selection of exemplary heterologous gene modifying polypeptides

| SEQ ID NO: | | | |
|---|---|---|---|
| for Full | | | |
| Polypeptide | | SEQ ID NO: | |
| Sequence | Linker Sequence | of linker | RT name |
| 2311 | GGGGSGGGGSGGGGSGGGGS | 15,405 | MLVCB_P08361_3mutA |
| 1373 | GGGGSGGGGSGGGGSGGGGSGGGGSGGGGS | 15,406 | AVIRE_P03360_3mutA |
| 2644 | GGGGSGGGGSGGGGSGGGGSGGGGSGGGGS | 15,407 | MLVMS_P03355_PLV919 |
| 2304 | GSSGSSGSSGSSGSSGSS | 15,408 | MLVCB_P08361_3mutA |
| 2325 | EAAAKEAAAKEAAAKEAAAK | 15,409 | MLVCB_P08361_3mutA |
| 2322 | EAAAKEAAAKEAAAKEAAAKEAAAKEAAAK | 15,410 | MLVCB_P08361_3mutA |
| 2187 | PAPAPAPAPAP | 15,411 | MLVBM_Q7SVK7_3mut |
| 2309 | PAPAPAPAPAPAP | 15,412 | MLVCB_P08361_3mutA |
| 2534 | PAPAPAPAPAPAP | 15,413 | MLVFF_P26809_3mutA |
| 2797 | PAPAPAPAPAPAP | 15,414 | MLVMS_P03355_3mutA_WS |
| 3084 | PAPAPAPAPAPAP | 15,415 | MLVMS_P03355_3mutA_WS |
| 2868 | PAPAPAPAPAPAP | 15,416 | MLVMS_P03355_PLV919 |
| 126 | EAAAKGGG | 15,417 | PERV_Q4VFZ2_3mut |
| 306 | EAAAKGGG | 15,418 | PERV_Q4VFZ2_3mut |
| 1410 | PAPGGG | 15,419 | AVIRE_P03360_3mutA |
| 804 | GGGGSSGGS | 15,420 | WMSV_P03359_3mut |
| 1937 | GGGGGSEAAAK | 15,421 | BAEVM_P10272_3mutA |
| 2721 | GGGEAAAKGGS | 15,422 | MLVMS_P03355_3mut |
| 3018 | GGGEAAAKGGS | 15,423 | MLVMS_P03355_3mut |
| 1018 | GGGEAAAKGGS | 15,424 | XMRV6_A1Z651_3mutA |
| 2317 | GGSGGGPAP | 15,425 | MLVCB_P08361_3mutA |
| 2649 | PAPGGSGGG | 15,426 | MLVMS_P03355_PLV919 |
| 2878 | PAPGGSGGG | 15,427 | MLVMS_P03355_PLV919 |
| 912 | GGSEAAAKPAP | 15,428 | WMSV_P03359_3mutA |
| 2338 | GGSPAPEAAAK | 15,429 | MLVCB_P08361_3mutA |
| 2527 | GGSPAPEAAAK | 15,430 | MLVFF_P26809_3mutA |
| 141 | EAAAKGGSPAP | 15,431 | PERV_Q4VFZ2_3mut |
| 341 | EAAAKGGSPAP | 15,432 | PERV_Q4VFZ2_3mut |
| 2315 | EAAAKPAPGGS | 15,433 | MLVCB_P08361_3mutA |
| 3080 | EAAAKPAPGGS | 15,434 | MLVMS_P03355_3mutA_WS |
| 2688 | GGGGSSEAAAK | 15,435 | MLVMS_P03355_PLV919 |
| 2885 | GGGGSSEAAAK | 15,436 | MLVMS_P03355_PLV919 |
| 2810 | GSSGGGEAAAK | 15,437 | MLVMS_P03355_3mutA_WS |
| 3057 | GSSGGGEAAAK | 15,438 | MLVMS_P03355_3mutA_WS |
| 1861 | GSSEAAAKGGG | 15,439 | MLVAV_P03356_3mutA |
| 3056 | GSSGGGPAP | 15,440 | MLVMS_P03355_3mutA_WS |
| 1038 | GSSPAPGGG | 15,441 | XMRV6_A1Z651_3mutA |
| 2308 | PAPGGGGSS | 15,442 | MLVCB_P08361_3mutA |
| 1672 | GGGEAAAKPAP | 15,443 | KORV_Q9TTC1-Pro_3mutA |
| 2526 | GGGEAAAKPAP | 15,444 | MLVFF_P26809_3mutA |
| 1938 | GGGPAPEAAAK | 15,445 | BAEVM_P10272_3mutA |
| 2641 | GSSEAAAKPAP | 15,446 | MLVMS_P03355_PLV919 |
| 2891 | GSSEAAAKPAP | 15,447 | MLVMS_P03355_PLV919 |
| 1225 | GSSPAPEAAAK | 15,448 | FLV_P10273_3mutA |
| 2839 | GSSPAPEAAAK | 15,449 | MLVMS_P03355_3mutA_WS |
| 3127 | GSSPAPEAAAK | 15,450 | MLVMS_P03355_3mutA_WS |
| 2798 | PAPGSSEAAAK | 15,451 | MLVMS_P03355_3mutA_WS |
| 3091 | PAPGSSEAAAK | 15,452 | MLVMS_P03355_3mutA_WS |
| 1372 | AEAAAKEAAAKEAAAKEAAAKALEAEAAAKEAA | 15,453 | AVIRE_P03360_3mutA |
| | AKEAAAKEAAAKA | | |
| 1197 | AEAAAKEAAAKEAAAKEAAAKALEAEAAAKEAA | 15,454 | FLV_P10273_3mutA |
| | AKEAAAKEAAAKA | | |
| 2611 | AEAAAKEAAAKEAAAKEAAAKALEAEAAAKEAA | 15,455 | MLVMS_P03355_PLV919 |
| | AKEAAAKEAAAKA | | |
| 2784 | AEAAAKEAAAKEAAAKEAAAKALEAEAAAKEAA | 15,456 | MLVMS_P03355_3mutA_WS |
| | AKEAAAKEAAAKA | | |
| 480 | AEAAAKEAAAKEAAAKEAAAKALEAEAAAKEAA | 15,457 | SFV1_P23074_2mutA |
| | AKEAAAKEAAAKA | | |
| 647 | AEAAAKEAAAKEAAAKEAAAKALEAEAAAKEAA | 15,458 | SFV3L_P27401_2mutA |
| | AKEAAAKEAAAKA | | |
| 1006 | AEAAAKEAAAKEAAAKEAAAKALEAEAAAKEAA | 15,459 | XMRV6_A1Z651_3mutA |
| | AKEAAAKEAAAKA | | |
| 2518 | SGSETPGTSESATPES | 15,460 | MLVFF_P26809_3mutA |

### Subsequences of Exemplary Heterologous Gene Modifying Polypeptides

In some embodiments, the heterologous gene modifying polypeptide comprises, in N-terminal to C-terminal order, one or more (e.g., 1, 2, 3, 4, 5, or all 6) of an N-terminal methionine residue, a first nuclear localization signal (NLS), a DNA binding domain, a linker, an RT domain, and/or a second NLS. In some embodiments, a heterologous gene modifying polypeptide comprises, in N-terminal to C-terminal order, a NLS (e.g., a first NLS), a DNA binding domain, a linker, and an RT domain, wherein the linker and RT domain are the linker and RT domain of a heterologous gene modifying polypeptide of any one of SEQ ID NOs: 1-7743, or an amino acid sequence having at least 70%, 75%, 80%, 85%, 90%, 95%, or 99% identity to said linker and RT domain. In some embodiments, a heterologous gene modifying polypeptide comprises, in N-terminal to C-terminal order, a DNA binding domain, a linker, an RT domain, and an NLS (e.g., a second NLS) wherein the linker and RT domain are the linker and RT domain of a heterologous gene modifying polypeptide of any one of SEQ ID NOs: 1-7743, or an amino acid sequence having at least 70%, 75%, 80%, 85%, 90%, 95%, or 99% identity to said linker and RT domain. In some embodiments, a heterologous gene modifying polypeptide comprises, in N-terminal to C-terminal order, a first NLS, a DNA binding domain, a linker, an RT domain, and a second NLS, wherein the linker and RT domain are the linker and RT domain of a heterologous gene modifying polypeptide of any one of SEQ ID NOs: 1-7743, or an amino acid sequence having at least 70%, 75%, 80%, 85%, 90%, 95%, or 99% identity to said linker and RT domain. In some embodiments, the heterologous gene modifying polypeptide further comprises an N-terminal methionine residue.

In some embodiments, the heterologous gene modifying polypeptide comprises, in N-terminal to C-terminal order, one or more (e.g., 1, 2, 3, 4, 5, or all 6) of an N-terminal methionine residue, a first nuclear localization signal (NLS) (e.g., of a heterologous gene modifying polypeptide of any one of SEQ ID NOs: 1-7743 and/or as listed in any of Tables A1, T1, or T2, or an amino acid sequence having at least 70%, 75%, 80%, 85%, 90%, 95%, or 99% identity thereto), a DNA binding domain (e.g., a Cas domain, e.g., a SpyCas9 domain, e.g., as listed in Table 8, or an amino acid sequence having at least 70%, 75%, 80%, 85%, 90%, 95%, or 99% identity thereto; or a DNA binding domain of a heterologous gene modifying polypeptide of any one of SEQ ID NOs: 1-7743 and/or as listed in any of Tables A1, T1, or T2, or an amino acid sequence having at least 70%, 75%, 80%, 85%, 90%, 95%, or 99% identity thereto), a linker (e.g., of a heterologous gene modifying polypeptide of any one of SEQ ID NOs: 1-7743 and/or as listed in any of Tables A1, T1, or T2, or an amino acid sequence having at least 70%, 75%, 80%, 85%, 90%, 95%, or 99% identity thereto), an RT domain (e.g., of a heterologous gene modifying polypeptide of any one of SEQ ID NOs: 1-7743 and/or as listed in any of Tables A1, T1, or T2, or an amino acid sequence having at least 70%, 75%, 80%, 85%, 90%, 95%, or 99% identity thereto), and a second NLS (e.g., of a heterologous gene modifying polypeptide of any one of SEQ ID NOs: 1-7743 and/or as listed in any of Tables A1, T1, or T2, or an amino acid sequence having at least 70%, 75%, 80%, 85%, 90%, 95%, or 99% identity thereto). In some embodiments, the heterologous gene modifying polypeptide further comprises (e.g., C-terminal to the second NLS) a T2A sequence and/or a puromycin sequence (e.g., of a heterologous gene modifying polypeptide of any one of SEQ ID NOs: 1-7743 and/or as listed in any of Tables A1, T1, or T2, or an amino acid sequence having at least 70%, 75%, 80%, 85%, 90%, 95%, or 99% identity thereto). In some embodiments, a nucleic acid encoding a heterologous gene modifying polypeptide (e.g., as described herein) encodes a T2A sequence, e.g., wherein the T2A sequence is situated between a region encoding the heterologous gene modifying polypeptide and a second region, wherein the second region optionally encodes a selectable marker, e.g., puromycin.

In certain embodiments, the first NLS comprises a first NLS sequence of a heterologous gene modifying polypeptide having an amino acid sequence of any one of SEQ ID NOs: 1-7743, or an amino acid sequence having at least 70%, 75%, 80%, 85%, 90%, 95%, or 99% identity thereto. In certain embodiments, the first NLS comprises a first NLS sequence of a heterologous gene modifying polypeptide as listed in any of Tables A1, T1, or T2, or an amino acid sequence having at least 70%, 75%, 80%, 85%, 90%, 95%, or 99% identity thereto. In certain embodiments, the first NLS sequence comprises a C-myc NLS. In certain embodiments, the first NLS comprises the amino acid sequence PAAKRVKLD (SEQ ID NO: 11,095), or an amino acid sequence having at least 70%, 75%, 80%, 85%, 90%, 95%, or 99% identity thereto.

In certain embodiments, the heterologous gene modifying polypeptide further comprises a spacer sequence between the first NLS and the DNA binding domain. In certain embodiments, the spacer sequence between the first NLS and the DNA binding domain comprises 1, 2, 3, 4, 5, 6, 7, 8, 9, or 10 amino acids. In certain embodiments, the spacer sequence between the first NLS and the DNA binding domain comprises the amino acid sequence GG.

In certain embodiments, the DNA binding domain comprises a DNA binding domain of a heterologous gene modifying polypeptide of any one of SEQ ID NOs: 1-7743, or an amino acid sequence having at least 70%, 75%, 80%, 85%, 90%, 95%, or 99% identity thereto. In certain embodiments, the DNA binding domain comprises a DNA binding domain of a heterologous gene modifying polypeptide as listed in any of Tables A1, T1, or T2, or an amino acid sequence having at least 70%, 75%, 80%, 85%, 90%, 95%, or 99% identity thereto. In certain embodiments, the DNA binding domain comprises a Cas domain (e.g., as listed in Table 8). In certain embodiments, the DNA binding domain comprises the amino acid sequence of a SpyCas9 polypeptide (e.g., as listed in Table 8, e.g., a Cas9 N863A polypeptide), or an amino acid sequence having at least 70%, 75%, 80%, 85%, 90%, 95%, or 99% identity thereto. In certain embodiments, the DNA binding domain comprises the amino acid sequence:

(SEQ ID NO: 11,096)
DKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNEDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKARGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD,

or an amino acid sequence having at least 70%, 75%, 80%, 85%, 90%, 95%, or 99% identity thereto.

In certain embodiments, the heterologous gene modifying polypeptide further comprises a spacer sequence between the DNA binding domain and the linker. In certain embodiments, the spacer sequence between the DNA binding domain and the linker comprises 1, 2, 3, 4, 5, 6, 7, 8, 9, or 10 amino acids. In certain embodiments, the spacer sequence between the DNA binding domain and the linker comprises the amino acid sequence GG.

In certain embodiments, the linker comprises a linker sequence of a heterologous gene modifying polypeptide of any one of SEQ ID NOs: 1-7743, or an amino acid sequence having at least 70%, 75%, 80%, 85%, 90%, 95%, or 99% identity thereto. In certain embodiments, the linker comprises a linker sequence of a heterologous gene modifying polypeptide as listed in any of Tables A1, T1, or T2, or an amino acid sequence having at least 70%, 75%, 80%, 85%, 90%, 95%, or 99% identity thereto. In certain embodiments, the linker comprises an amino acid sequence as listed in Table D or 10, or an amino acid sequence having at least 70%, 75%, 80%, 85%, 90%, 95%, or 99% identity thereto.

In certain embodiments, the heterologous gene modifying polypeptide further comprises a spacer sequence between the linker and the RT domain. In certain embodiments, the spacer sequence between the linker and the RT domain comprises 1, 2, 3, 4, 5, 6, 7, 8, 9, or 10 amino acids. In certain embodiments, the spacer sequence between the linker and the RT domain comprises the amino acid sequence GG.

In certain embodiments, the RT domain comprises a RT domain sequence of a heterologous gene modifying polypeptide of any one of SEQ ID NOs: 1-7743, or an amino acid sequence having at least 70%, 75%, 80%, 85%, 90%, 95%, or 99% identity thereto. In certain embodiments, the RT domain comprises a RT domain sequence of a heterologous gene modifying polypeptide as listed in any of Tables A1, T1, or T2, or an amino acid sequence having at least 70%, 75%, 80%, 85%, 90%, 95%, or 99% identity thereto. In certain embodiments, the RT domain comprises an amino acid sequence as listed in Table D or 6, or an amino acid sequence having at least 70%, 75%, 80%, 85%, 90%, 95%, or 99% identity thereto. In some embodiments, the RT domain has a length of about 400-500, 500-600, 600-700, 700-800, 800-900, or 900-1000 amino acids.

In certain embodiments, the heterologous gene modifying polypeptide further comprises a spacer sequence between the RT domain and the second NLS. In certain embodiments, the spacer sequence between the RT domain and the second NLS comprises 1, 2, 3, 4, 5, 6, 7, 8, 9, or 10 amino acids. In certain embodiments, the spacer sequence between the RT domain and the second NLS comprises the amino acid sequence AG.

In certain embodiments, the second NLS comprises a second NLS sequence of a heterologous gene modifying polypeptide of any one of SEQ ID NOs: 1-7743. In certain embodiments, the second NLS comprises a second NLS sequence of a heterologous gene modifying polypeptide as listed in any of Tables A1, T1, or T2. In certain embodiments, the second NLS sequence comprises a plurality of partial NLS sequences. In embodiments, the NLS sequence, e.g., the second NLS sequence, comprises a first partial NLS sequence, e.g., comprising the amino acid sequence KRTADGSEFE (SEQ ID NO: 11,097), or an amino acid sequence having at least 70%, 75%, 80%, 85%, 90%, 95%, or 99% identity thereto. In embodiments, the NLS sequence, e.g., the second NLS sequence, comprises a second partial NLS sequence. In embodiments, the NLS sequence, e.g., the second NLS sequence, comprises an SV40A5 NLS, e.g., a bipartite SV40A5 NLS, e.g., comprising the amino acid sequence KRTADGSEFESPKKKAKVE (SEQ ID NO: 11,098), or an amino acid sequence having at least 70%, 75%, 80%, 85%, 90%, 95%, or 99% identity thereto. In certain embodiments, the NLS sequence, e.g., the second NLS sequence, comprises the amino acid sequence KRTADGSEFEKRTADGSEFESPKKKAKVE (SEQ ID NO: 11,099), or an amino acid sequence having at least 70%, 75%, 80%, 85%, 90%, 95%, or 99% identity thereto.

In certain embodiments, the heterologous gene modifying polypeptide further comprises a spacer sequence between the second NLS and the T2A sequence and/or puromycin sequence. In certain embodiments, the spacer sequence between the second NLS and the T2A sequence and/or puromycin sequence comprises 1, 2, 3, 4, 5, 6, 7, 8, 9, or 10 amino acids. In certain embodiments, the spacer sequence between the second NLS and the T2A sequence and/or puromycin sequence comprises the amino acid sequence GSG.

### Linkers and RT Domains

In some embodiments, the heterologous gene modifying polypeptide comprises a linker (e.g., as described herein) and an RT domain (e.g., as described herein). In certain embodiments, the heterologous gene modifying polypeptide comprises, in N-terminal to C-terminal order, a linker (e.g., as described herein) and an RT domain (e.g., as described herein).

In certain embodiments, the linker comprises a linker sequence as listed in Table 10, or an amino acid sequence having at least 70%, 75%, 80%, 85%, 90%, 95%, or 99% identity thereto. In certain embodiments, the linker comprises a linker sequence of any one of SEQ ID NOs: 1-7743, or an amino acid sequence having at least 70%, 75%, 80%, 85%, 90%, 95%, or 99% identity thereto. In certain embodiments, the linker comprises a linker sequence of any one of SEQ ID NOs: 6001-7743, or an amino acid sequence having at least 70%, 75%, 80%, 85%, 90%, 95%, or 99% identity thereto. In certain embodiments, the linker comprises a linker sequence of any one of SEQ ID NOs: 4501-4541, or an amino acid sequence having at least 70%, 75%, 80%, 85%, 90%, 95%, or 99% identity thereto. In certain embodiments, the linker comprises a linker sequence of an exemplary heterologous gene modifying polypeptide listed in any of Tables A1, T1, or T2, or an amino acid sequence having at least 70%, 75%, 80%, 85%, 90%, 95%, or 99% identity thereto. In certain embodiments, the RT domain comprises an RT domain sequence as listed in Table 6, or an amino acid sequence having at least 70%, 75%, 80%, 85%, 90%, 95%, or 99% identity thereto. In certain embodiments, the RT domain comprises an RT domain sequence of an exemplary heterologous gene modifying polypeptide listed in any of Tables A1, T1, or T2, or an amino acid sequence having at least 70%, 75%, 80%, 85%, 90%, 95%, or 99% identity thereto.

In some embodiments, a heterologous gene modifying polypeptide comprises a portion of a heterologous gene modifying polypeptide of any one of SEQ ID NOs: 1-7743, wherein the portion comprises a linker and RT domain, or an amino acid sequence having at least 70%, 75%, 80%, 85%, 90%, 95%, or 99% identity to said portion.

In some embodiments, a heterologous gene modifying polypeptide comprises a linker of a heterologous gene modifying polypeptide of any one of SEQ ID NOs: 1-7743, or an amino acid sequence having at least 70%, 75%, 80%, 85%, 90%, 95%, or 99% identity to said linker. In some embodiments, a heterologous gene modifying polypeptide comprises a linker of a heterologous gene modifying polypeptide of any one of SEQ ID NOs: 6001-7743, or an amino acid sequence having at least 70%, 75%, 80%, 85%, 90%, 95%, or 99% identity to said linker. In some embodiments, a heterologous gene modifying polypeptide comprises a linker of a heterologous gene modifying polypeptide of any one of SEQ ID NOs: 4501-4541, or an amino acid sequence having at least 70%, 75%, 80%, 85%, 90%, 95%, or 99% identity to said linker. In some embodiments, a heterologous gene modifying polypeptide comprises a linker of a heterologous gene modifying polypeptide as listed in any of Tables A1, T1, or T2, or a linker comprising an amino acid sequence having at least 70%, 75%, 80%, 85%, 90%, 95%, or 99% identity thereto.

In some embodiments, a heterologous gene modifying polypeptide comprises an RT domain of a heterologous gene modifying polypeptide of any one of SEQ ID NOs: 1-7743, or an amino acid sequence having at least 70%, 75%, 80%, 85%, 90%, 95%, or 99% identity to said RT domain. In some embodiments, a heterologous gene modifying polypeptide comprises an RT domain of a heterologous gene modifying polypeptide of any one of SEQ ID NOs: 6001-7743, or an amino acid sequence having at least 70%, 75%, 80%, 85%, 90%, 95%, or 99% identity said RT domain. In some embodiments, a heterologous gene modifying polypeptide comprises an RT domain of a heterologous gene modifying polypeptide of any one of SEQ ID NOs: 4501-4541, or an amino acid sequence having at least 70%, 75%, 80%, 85%, 90%, 95%, or 99% identity said RT domain. In some embodiments, a heterologous gene modifying polypeptide comprises an RT domain of a heterologous gene modifying polypeptide as listed in any of Tables A1, T1, or T2, or an RT domain comprising an amino acid sequence having at least 70%, 75%, 80%, 85%, 90%, 95%, or 99% identity thereto.

In certain embodiments, the linker and the RT domain of a heterologous gene modifying polypeptide comprise the amino acid sequences of a linker and RT domain (or amino acid sequences having at least 70%, 75%, 80%, 85%, 90%, 95%, or 99% identity thereto) of a heterologous gene modifying polypeptide having the amino acid sequence of any one of SEQ ID NOs: 1-7743. In certain embodiments, the linker and the RT domain of a heterologous gene modifying polypeptide comprise amino acid sequences of a linker and RT domain having at least 80% identity to the linker and RT domains of any one of SEQ ID NOs: 1-7743. In certain embodiments, the linker and the RT domain of a heterologous gene modifying polypeptide comprise amino acid sequences of a linker and RT domain having at least 90% identity to the linker and RT domains of any one of SEQ ID NOs: 1-7743. In certain embodiments, the linker and the RT domain of a heterologous gene modifying polypeptide comprise amino acid sequences of a linker and RT domain having at least 95% identity to the linker and RT domains of any one of SEQ ID NOs: 1-7743. In certain embodiments, the linker and the RT domain of a heterologous gene modifying polypeptide comprise amino acid sequences of a linker and RT domain having at least 99% identity to the linker and RT domains of any one of SEQ ID NOs: 1-7743. In certain embodiments, the linker and the RT domain of a heterologous gene modifying polypeptide comprise the amino acid sequences of a linker and RT domain (or amino acid sequences having at least 70%, 75%, 80%, 85%, 90%, 95%, or 99% identity thereto) of a heterologous gene modifying polypeptide having the amino acid sequence of any one of SEQ ID NOs: 6001-7743. In certain embodiments, the linker and the RT domain of a heterologous gene modifying polypeptide comprise the amino acid sequences of a linker and RT domain (or amino acid sequences having at least 70%, 75%, 80%, 85%, 90%, 95%, or 99% identity thereto) of a heterologous gene modifying polypeptide having the amino acid sequence of any one of SEQ ID NOs: 4501-4541. In certain embodiments, the linker and the RT domain of a heterologous gene modifying polypeptide comprise the amino acid sequences of a linker and RT domain (or amino acid sequences having at least 70%, 75%, 80%, 85%, 90%, 95%, or 99% identity thereto) from a single row of any of Tables A1, T1, or T2 (e.g., from a single exemplary heterologous gene modifying polypeptide as listed in any of Tables A1, T1, or T2).

In certain embodiments, the linker and the RT domain of a heterologous gene modifying polypeptide comprise the amino acid sequences of a linker and RT domain (or amino acid sequences having at least 70%, 75%, 80%, 85%, 90%, 95%, or 99% identity thereto) from two different amino acid sequences selected from SEQ ID NOs: 1-7743. In certain embodiments, the linker and the RT domain of a heterologous gene modifying polypeptide comprise the amino acid sequences of a linker and RT domain (or amino acid sequences having at least 70%, 75%, 80%, 85%, 90%, 95%, or 99% identity thereto) from different rows of any of Tables A1, T1, or T2.

In certain embodiments, the heterologous gene modifying polypeptide further comprises a first NLS (e.g., a 5′ NLS), e.g., as described herein. In certain embodiments, the heterologous gene modifying polypeptide further comprises a second NLS (e.g., a 3′ NLS), e.g., as described herein. In certain embodiments, the heterologous gene modifying polypeptide further comprises an N-terminal methionine residue.

### RT Families and Mutants

In certain embodiments, a heterologous gene modifying polypeptide comprises the amino acid sequence of an RT domain sequence from a family selected from: AVIRE, BAEVM, FFV, FLV, FOAMV, GALV, KORV, MLVAV, MLVBM, MLVCB, MLVFF, MLVMS, PERV, SFV1, SFV3L, WMSV, XMRV6, BLVAU, BLVJ, HTLIA, HTLIC, HTLIL, HTL32, HTL3P, HTLV2, JSRV, MLVF5, MLVRD, MMTVB, MPMV, SFVCP, SMRVH, SRV1, SRV2, and WDSV. In certain embodiments, a heterologous gene modifying polypeptide comprises comprises the amino acid sequence of an RT domain sequence from a family selected from: AVIRE, BAEVM, FFV, FLV, FOAMV, GALV, KORV, MLVAV, MLVBM, ML VCB, MLVFF, MLVMS, PERV, SFV1, SFV3L, WMSV, and XMRV6.

In certain embodiments, a heterologous gene modifying polypeptide comprises the amino acid sequence of an RT domain sequence from an MLVMS RT domain. In embodiments, the amino acid sequence of an RT domain sequence comprises one or more point mutations as listed in column 1 of Table M1, or a point mutation corresponding thereto. In embodiments, the amino acid sequence of an RT domain sequence comprises one or more point mutations as listed in column 3 of Table M1 (Gen1 MLVMS), or a point mutation corresponding thereto. In embodiments, the amino acid sequence of an RT domain sequence comprises one or more point mutations at an amino acid position of the RT domain as listed in columns 1 and 2 of Table M2, or an amino acid position corresponding thereto.

In certain embodiments, a heterologous gene modifying polypeptide comprises the amino acid sequence of an RT domain sequence from an AVIRE RT domain. In embodiments, the amino acid sequence of an RT domain sequence comprises one or more point mutations as listed in column 2 of Table M1, or a point mutation corresponding thereto. In embodiments, the amino acid sequence of an RT domain sequence comprises one or more point mutations as listed in column 4 of Table M1 (Gen2 AVIRE), or a point mutation corresponding thereto. In embodiments, the amino acid sequence of an RT domain sequence comprises one or more point mutations at an amino acid position of the RT domain as listed in columns 3 and 4 of Table M2, or an amino acid position corresponding thereto. In certain embodiments, the RT domain comprises an IENSSP (SEQ ID NO: 37639) (e.g., at the C-terminus).

TABLE M1
Exemplary point mutations in MLVMS and AVIRE RT domains

| RT-linker | Corres- | Gen1 | Gen2 |
|---|---|---|---|
| filing | ponding | MLVMS | AVIRE |
| (MLVMS) | AVIRE | (PLV4921) | (PLV10990) |
| | | H8Y | |
| P51L | Q51L | | |
| S67R | T67R | | |
| E67K | E67K | | |
| E69K | E69K | | |
| T197A | T197A | | |
| D200N | D200N | D200N | D200N |
| H204R | N204R | | |
| E302K | E302K | | |
| | | T306K | T306K |
| F309N | Y309N | | |
| W313F | W313F | W313F | W313F |
| T330P | G330P | T330P | G330P |
| L435G | T436G | | |
| N454K | N455K | | |
| D524G | D526G | | |
| E562Q | E564Q | | |
| D583N | D585N | | |
| H594Q | H596Q | | |
| L603W | L605W | L603W | L605W |
| D653N | D655N | | |
| L671P | L673P | | |

IENSSP (SEQ ID NO:
37639) at C-term

TABLE M2
Positions that can be mutated in exemplary
MLVMS and AVIRE RT domains
WT residue & position

| | MLVMS | | AVIRE |
|---|---|---|---|
| | position # | | position # |
| MLVMS aa | * | AVIRE aa | * |
| H | 8 | Y | 8 |
| P | 51 | Q | 51 |
| S | 67 | T | 67 |
| E | 69 | E | 69 |
| T | 197 | T | 197 |
| D | 200 | D | 200 |
| H | 204 | N | 204 |
| E | 302 | E | 302 |
| T | 306 | T | 306 |
| F | 309 | Y | 309 |
| W | 313 | W | 313 |
| T | 330 | G | 330 |
| L | 435 | T | 436 |
| N | 454 | N | 455 |
| D | 524 | D | 526 |
| E | 562 | E | 564 |
| D | 583 | D | 585 |
| H | 594 | H | 596 |
| L | 603 | L | 605 |
| D | 653 | D | 655 |
| L | 671 | S | 673 |

In certain embodiments, a heterologous gene modifying polypeptide comprises a gamma retrovirus derived RT domain. In certain embodiments, the gamma retrovirus-derived RT domain of a heterologous gene modifying polypeptide comprises the amino acid sequence of an RT domain sequence from a family selected from: AVIRE, BAEVM, FFV, FLV, FOAMV, GALV, KORV, MLVAV, MLVBM, MLVCB, MLVFF, MLVMS, PERV, SFV1, SFV3L, WMSV, and XMRV6. In some embodiments, the gamma retrovirus-derived RT domain of a heterologous gene modifying polypeptide is not derived from PERV. In some embodiments, said RT includes one, two, three, four, five, six or more mutations shown in Table 2A1 and corresponding to mutations D200N, L603W, T330P, D524G, E562Q, D583N, P51L, S67R, E67K, T197A, H204R, E302K, F309N, W313F, L435G, N454K, H594Q, L671P, E69K, or D653N in the RT domain of murine leukemia virus reverse transcriptase. In some embodiments, the heterologous gene modifying polypeptide further comprises a linker having at least 99% identity to a linker domains of any one of SEQ ID NOs: 1-7743. In some embodiments, the heterologous gene modifying polypeptide further comprises a linker having at least 99% or 100% identity to SEQ ID NO: 5217 or SEQ ID NO:11,041.

In embodiments, the RT domain comprises the amino acid sequence of an RT domain of an AVIRE RT (e.g., an AVIRE_P03360 sequence, e.g., SEQ ID NO: 8001), or an amino acid sequence having at least 70%, 75%, 80%, 85%, 90%, 95%, or 99% identity thereto. In some embodiments, the RT domain comprises the amino acid sequence of an AVIRE RT further comprising one, two, three, four, or five mutations selected from the group consisting of D200N, G330P, L605W, T306K, and W313F, or a corresponding position in a homologous RT domain. In some embodiments, the RT domain comprises the amino acid sequence of an AVIRE RT further comprising one, two, or three mutations selected from the group consisting of D200N, G330P, and L605W, or a corresponding position in a homologous RT domain.

In embodiments, the RT domain comprises the amino acid sequence of an RT domain of a BAEVM RT (e.g., an BAEVM_P10272 sequence, e.g., SEQ ID NO: 8004), or an amino acid sequence having at least 70%, 75%, 80%, 85%, 90%, 95%, or 99% identity thereto. In some embodiments, the RT domain comprises the amino acid sequence of a BAEVM RT further comprising one, two, three, four, or five mutations selected from the group consisting of D198N, E328P, L602W, T304K, and W311F, or a corresponding position in a homologous RT domain. In some embodiments, the RT domain comprises the amino acid sequence of a BAEVM RT further comprising one, two, or three mutations selected from the group consisting of D198N, E328P, and L602W, or a corresponding position in a homologous RT domain.

In embodiments, the RT domain comprises the amino acid sequence of an RT domain of an FFV RT (e.g., an FFV_093209 sequence, e.g., SEQ ID NO: 8012), or an amino acid sequence having at least 70%, 75%, 80%, 85%, 90%, 95%, or 99% identity thereto. In some embodiments, the RT domain comprises the amino acid sequence of an FFV RT further comprising one, two, three, or four mutations selected from the group consisting of D21N, T293N, T419P, and L393K, or a corresponding position in a homologous RT domain. In some embodiments, the RT domain comprises the amino acid sequence of an FFV RT further comprising one, two, or three mutations selected from the group consisting of D21N, T293N, and T419P, or a corresponding position in a homologous RT domain. In some embodiments, the RT domain comprises the amino acid sequence of an FFV RT further comprising the mutation D21N. In some embodiments, the RT domain comprises the amino acid sequence of an FFV RT further comprising one, two, or three mutations selected from the group consisting of T207N, T333P, and L307K, or a corresponding position in a homologous RT domain. In some embodiments, the RT domain comprises the amino acid sequence of an FFV RT further comprising one or two mutations selected from the group consisting of T207N and T333P, or a corresponding position in a homologous RT domain.

In embodiments, the RT domain comprises the amino acid sequence of an RT domain of an FLV RT (e.g., an FLV_P10273 sequence, e.g., SEQ ID NO: 8019), or an amino acid sequence having at least 70%, 75%, 80%, 85%, 90%, 95%, or 99% identity thereto. In some embodiments, the RT domain comprises the amino acid sequence of an FLV RT further comprising one, two, three, or four mutations selected from the group consisting of D199N, L602W, T305K, and W312F, or a corresponding position in a homologous RT domain. In some embodiments, the RT domain comprises the amino acid sequence of an FLV RT further comprising one or two mutations selected from the group consisting of D199N and L602W, or a corresponding position in a homologous RT domain.

In embodiments, the RT domain comprises the amino acid sequence of an RT domain of a FOAMV RT (e.g., an FOAMV_P14350 sequence, e.g., SEQ ID NO: 8021), or an amino acid sequence having at least 70%, 75%, 80%, 85%, 90%, 95%, or 99% identity thereto. In some embodiments, the RT domain comprises the amino acid sequence of an FOAMV RT further comprising one, two, three, or four mutations selected from the group consisting of D24N, T296N, S420P, and L396K, or a corresponding position in a homologous RT domain. In some embodiments, the RT domain comprises the amino acid sequence of an FOAMV RT further comprising one, two, or three mutations selected from the group consisting of D24N, T296N, and S420P, or a corresponding position in a homologous RT domain. In some embodiments, the RT domain comprises the amino acid sequence of an FOAMV RT further comprising the mutation D24N, or a corresponding position in a homologous RT domain. In some embodiments, the RT domain comprises the amino acid sequence of an FOAMV RT further comprising one, two, or three mutations selected from the group consisting of T207N, S331P, and L307K, or a corresponding position in a homologous RT domain. In some embodiments, the RT domain comprises the amino acid sequence of an FOAMV RT further comprising one or two mutations selected from the group consisting of T207N and S331P, or a corresponding position in a homologous RT domain.

In embodiments, the RT domain comprises the amino acid sequence of an RT domain of a GALV RT (e.g., an GALV_P21414 sequence, e.g., SEQ ID NO: 8027), or an amino acid sequence having at least 70%, 75%, 80%, 85%, 90%, 95%, or 99% identity thereto. In some embodiments, the RT domain comprises the amino acid sequence of a GALV RT further comprising one, two, three, four, or five mutations selected from the group consisting of D198N, E328P, L600W, T304K, and W311F, or a corresponding position in a homologous RT domain. In some embodiments, the RT domain comprises the amino acid sequence of a GALV RT further comprising one, two, or three mutations selected from the group consisting of D198N, E328P, and L600W, or a corresponding position in a homologous RT domain.

In embodiments, the RT domain comprises the amino acid sequence of an RT domain of a KORV RT (e.g., an KORV_Q9TTC1 sequence, e.g., SEQ ID NO: 8047), or an amino acid sequence having at least 70%, 75%, 80%, 85%, 90%, 95%, or 99% identity thereto. In some embodiments, the RT domain comprises the amino acid sequence of a GALV RT further comprising one, two, three, four, five, or six mutations selected from the group consisting of D32N, D322N, E452P, L274W, T428K, and W435F, or a corresponding position in a homologous RT domain. In some embodiments, the RT domain comprises the amino acid sequence of a GALV RT further comprising one, two, three, or four mutations selected from the group consisting of D32N, D322N, E452P, and L274W, or a corresponding position in a homologous RT domain. In some embodiments, the RT domain comprises the amino acid sequence of a GALV RT further comprising the mutation D32N. In some embodiments, the RT domain comprises the amino acid sequence of a KORV RT further comprising one, two, three, four, or five mutations selected from the group consisting of D23IN, E361P, L633W, T337K, and W344F, or a corresponding position in a homologous RT domain. In some embodiments, the RT domain comprises the amino acid sequence of a KORV RT further comprising one, two, or three mutations selected from the group consisting of D231N, E361P, and L633W, or a corresponding position in a homologous RT domain.

In embodiments, the RT domain comprises the amino acid sequence of an RT domain of a MLVAV RT (e.g., an MLVAV_P03356 sequence, e.g., SEQ ID NO: 8053), or an amino acid sequence having at least 70%, 75%, 80%, 85%, 90%, 95%, or 99% identity thereto. In some embodiments, the RT domain comprises the amino acid sequence of a MLVAV RT further comprising one, two, three, four, or five mutations selected from the group consisting of D200N, T330P, L603W, T306K, and W313F, or a corresponding position in a homologous RT domain. In some embodiments, the RT domain comprises the amino acid sequence of a MLVAV RT further comprising one, two, or three mutations selected from the group consisting of D200N, T330P, and L603W, or a corresponding position in a homologous RT domain.

In embodiments, the RT domain comprises the amino acid sequence of an RT domain of a MLVBM RT (e.g., an MLVBM_Q7SVK7 sequence, e.g., SEQ ID NO: 8056), or an amino acid sequence having at least 70%, 75%, 80%, 85%, 90%, 95%, or 99% identity thereto. In some embodiments, the RT domain comprises the amino acid sequence of a MLVBM RT further comprising one, two, three, four, or five mutations selected from the group consisting of D199N, T329P, L602W, T305K, and W312F, or a corresponding position in a homologous RT domain. In some embodiments, the RT domain comprises the amino acid sequence of a ML VBM RT further comprising one, two, and three mutations selected from the group consisting of D200N, T330P, and L603W, or a corresponding position in a homologous RT domain.

In embodiments, the RT domain comprises the amino acid sequence of an RT domain of a MLVCB RT (e.g., an MLVCB_P08361 sequence, e.g., SEQ ID NO: 8062), or an amino acid sequence having at least 70%, 75%, 80%, 85%, 90%, 95%, or 99% identity thereto. In some embodiments, the RT domain comprises the amino acid sequence of a MLVCB RT further comprising one, two, three, four, or five mutations selected from the group consisting of D200N, T330P, L603W, T306K, and W313F, or a corresponding position in a homologous RT domain. In some embodiments, the RT domain comprises the amino acid sequence of a ML VCB RT further comprising one, two, and three mutations selected from the group consisting of D200N, T330P, and L603W, or a corresponding position in a homologous RT domain.

In embodiments, the RT domain comprises the amino acid sequence of an RT domain of a MLVFF RT, or an amino acid sequence having at least 70%, 75%, 80%, 85%, 90%, 95%, or 99% identity thereto. In some embodiments, the RT domain comprises the amino acid sequence of a ML VFF RT further comprising one, two, three, four, or five mutations selected from the group consisting of D200N, T330P, L603W, T306K, and W313F, or a corresponding position in a homologous RT domain. In some embodiments, the RT domain comprises the amino acid sequence of a MLVFF RT further comprising one, two, and three mutations selected from the group consisting of D200N, T330P, and L603W, or a corresponding position in a homologous RT domain.

In embodiments, the RT domain comprises the amino acid sequence of an RT domain of a MLVMS RT (e.g., an MLVMS_reference sequence, e.g., SEQ ID NO: 8137; or an MLVMS_P03355 sequence, e.g., SEQ ID NO: 8070), or an amino acid sequence having at least 70%, 75%, 80%, 85%, 90%, 95%, or 99% identity thereto. In some embodiments, the RT domain comprises the amino acid sequence of a MLVMS RT further comprising one, two, three, four, five, or six mutations selected from the group consisting of D200N, T330P, L603W, T306K, W313F, and H8Y, or a corresponding position in a homologous RT domain. In some embodiments, the RT domain comprises the amino acid sequence of a MLVMS RT further comprising one, two, three, four, or five mutations selected from the group consisting of D200N, T330P, L603W, T306K, and W313F, or a corresponding position in a homologous RT domain. In some embodiments, the RT domain comprises the amino acid sequence of a MLVMS RT further comprising one, two, or three mutations selected from the group consisting of D200N, T330P, and L603W, or a corresponding position in a homologous RT domain.

In embodiments, the RT domain comprises the amino acid sequence of an RT domain of a PERV RT (e.g., an PERV_Q4VFZ2 sequence, e.g., SEQ ID NO: 8099), or an amino acid sequence having at least 70%, 75%, 80%, 85%, 90%, 95%, or 99% identity thereto. In some embodiments, the RT domain comprises the amino acid sequence of a PERV RT further comprising one, two, three, four, or five mutations selected from the group consisting of D196N, E326P, L599W, T302K, and W309F, or a corresponding position in a homologous RT domain. In some embodiments, the RT domain comprises the amino acid sequence of a PERV RT further comprising one, two, or three mutations selected from the group consisting of D196N, E326P, and L599W, or a corresponding position in a homologous RT domain.

In embodiments, the RT domain comprises the amino acid sequence of an RT domain of a SFV1 RT (e.g., an SFV1_P23074 sequence, e.g., SEQ ID NO: 8105), or an amino acid sequence having at least 70%, 75%, 80%, 85%, 90%, 95%, or 99% identity thereto. In some embodiments, the RT domain comprises the amino acid sequence of a SFV1 RT further comprising one, two, three, or four mutations selected from the group consisting of D24N, T296N, N420P, and L396K, or a corresponding position in a homologous RT domain. In some embodiments, the RT domain comprises the amino acid sequence of a SFV1 RT further comprising one, two, or three mutations selected from the group consisting of D24N, T296N, and N420P, or a corresponding position in a homologous RT domain. In some embodiments, the RT domain comprises the amino acid sequence of a SFV1 RT further comprising the D24N, or a corresponding position in a homologous RT domain.

In embodiments, the RT domain comprises the amino acid sequence of an RT domain of a SFV3L RT (e.g., an SFV3L_P27401 sequence, e.g., SEQ ID NO: 8111), or an amino acid sequence having at least 70%, 75%, 80%, 85%, 90%, 95%, or 99% identity thereto. In some embodiments, the RT domain comprises the amino acid sequence of a SFV3L RT further comprising one, two, three, or four mutations selected from the group consisting of D24N, T296N, N422P, and L396K, or a corresponding position in a homologous RT domain. In some embodiments, the RT domain comprises the amino acid sequence of a SFV3L RT further comprising one, two, or three mutations selected from the group consisting of D24N, T296N, and N422P, or a corresponding position in a homologous RT domain. In some embodiments, the RT domain comprises the amino acid sequence of a SFV3L RT further comprising the mutation D24N, or a corresponding position in a homologous RT domain. In some embodiments, the RT domain comprises the amino acid sequence of a SFV3L RT further comprising one, two, or three mutations selected from the group consisting of T307N, N333P, and L307K, or a corresponding position in a homologous RT domain. In some embodiments, the RT domain comprises the amino acid sequence of a SFV3L RT further comprising one or two mutations selected from the group consisting of T307N and N333P, or a corresponding position in a homologous RT domain.

In embodiments, the RT domain comprises the amino acid sequence of an RT domain of a WMSV RT (e.g., an WMSV_P03359 sequence, e.g., SEQ ID NO: 8131), or an amino acid sequence having at least 70%, 75%, 80%, 85%, 90%, 95%, or 99% identity thereto. In some embodiments, the RT domain comprises the amino acid sequence of a WMSV RT further comprising one, two, three, four, or five mutations selected from the group consisting of D198N, E328P, L600W, T304K, and W311F, or a corresponding position in a homologous RT domain. In some embodiments, the RT domain comprises the amino acid sequence of a WMSV RT further comprising one, two, or three mutations selected from the group consisting of D198N, E328P, and L600W, or a corresponding position in a homologous RT domain.

In embodiments, the RT domain comprises the amino acid sequence of an RT domain of a XMRV6 RT (e.g., an XMRV6_A1Z651 sequence, e.g., SEQ ID NO: 8134), or an amino acid sequence having at least 70%, 75%, 80%, 85%, 90%, 95%, or 99% identity thereto. In some embodiments, the RT domain comprises the amino acid sequence of a XMRV6 RT further comprising one, two, three, four, or five mutations selected from the group consisting of D200N, T330P, L603W, T306K, and W313F, or a corresponding position in a homologous RT domain. In some embodiments, the RT domain comprises the amino acid sequence of a XMR V6 RT further comprising one, two, or three mutations selected from the group consisting of D200N, T330P, and L603W, or a corresponding position in a homologous RT domain.

In certain embodiments, the RT domain of a heterologous gene modifying polypeptide comprises the amino acid sequence of an RT domain of an AVIRE RT, or an amino acid sequence having at least 70%, 75%, 80%, 85%, 90%, 95%, or 99% identity thereto. In embodiments, the RT domain comprises the amino acid sequence of an RT domain comprised in a sequence listed in column 1 of Table A5, or an amino acid sequence having at least 70%, 75%, 80%, 85%, 90%, 95%, or 99% identity thereto. In some embodiments, the heterologous gene modifying polypeptide further comprises a linker having at least 99% or 100% identity to SEQ ID NO: 5217 or SEQ ID NO:11,041.

In certain embodiments, the RT domain of a heterologous gene modifying polypeptide comprises the amino acid sequence of an RT domain of an MLVMS RT, or an amino acid sequence having at least 70%, 75%, 80%, 85%, 90%, 95%, or 99% identity thereto. In embodiments, the RT domain comprises the amino acid sequence of an RT domain comprised in a sequence listed in any of columns 2-6 of Table A5, or an amino acid sequence having at least 70%, 75%, 80%, 85%, 90%, 95%, or 99% identity thereto. In some embodiments, the heterologous gene modifying polypeptide further comprises a linker having at least 99% or 100% identity to SEQ ID NO: 5217 or SEQ ID NO:11,041.

TABLE A5
Exemplary heterologous gene modifying
polypeptides comprising an AVIRE RT
domain or an MLVMS RT domain.

| AVIRE | | | | | |
|---|---|---|---|---|---|
| SEQ ID | | | | | |
| NOS: | MLVMS SEQ ID NOS: | | | | |
| 1 | 2704 | 3007 | 3038 | 2638 | 2930 |
| 2 | 2706 | 3007 | 3038 | 2639 | 2930 |
| 3 | 2708 | 3008 | 3039 | 2639 | 2931 |
| 4 | 2709 | 3008 | 3039 | 2640 | 2931 |
| 5 | 2709 | 3009 | 3040 | 2640 | 2932 |
| 6 | 2710 | 3010 | 3040 | 2641 | 2932 |
| 7 | 2957 | 3010 | 3041 | 2641 | 2933 |
| 9 | 2957 | 3011 | 3041 | 2642 | 2933 |
| 10 | 2958 | 3012 | 3042 | 2642 | 2934 |
| 12 | 2959 | 3012 | 3042 | 2643 | 2934 |
| 13 | 2960 | 3013 | 3043 | 2643 | 2935 |
| 14 | 2962 | 3013 | 3043 | 2644 | 2935 |
| 6076 | 6042 | 3014 | 3044 | 2644 | 2936 |
| 6143 | 6068 | 3014 | 3044 | 2645 | 2936 |
| 6200 | 6097 | 3015 | 3045 | 2645 | 2937 |
| 6254 | 6136 | 3015 | 3045 | 2646 | 2937 |
| 6274 | 6156 | 3016 | 3046 | 2646 | 2938 |
| 6315 | 6215 | 3016 | 3046 | 2647 | 2938 |
| 6328 | 6216 | 3017 | 3047 | 2647 | 2939 |
| 6337 | 6301 | 3018 | 3047 | 2648 | 2939 |
| 6403 | 6352 | 3018 | 3048 | 2648 | 2940 |
| 6420 | 6365 | 3019 | 3048 | 2649 | 2940 |
| 6440 | 6411 | 3019 | 3049 | 2649 | 2941 |
| 6513 | 6436 | 3020 | 3049 | 2650 | 2941 |
| 6552 | 6458 | 3020 | 3050 | 2650 | 2942 |
| 6613 | 6459 | 3021 | 3051 | 2651 | 2942 |
| 6671 | 6524 | 3021 | 3051 | 2651 | 2943 |
| 6822 | 6562 | 3022 | 3052 | 2652 | 2943 |
| 6840 | 6563 | 3023 | 3052 | 2652 | 2944 |
| 6884 | 6699 | 3023 | 3053 | 2653 | 2945 |
| 6907 | 6865 | 3024 | 3053 | 2653 | 2945 |
| 6970 | 7022 | 3024 | 3054 | 2654 | 2946 |
| 7025 | 7037 | 3025 | 3054 | 2655 | 2946 |
| 7052 | 7088 | 3025 | 3055 | 2655 | 2947 |
| 7078 | 7116 | 3026 | 3055 | 2656 | 2947 |
| 7243 | 7175 | 3026 | 3056 | 2656 | 2948 |
| 7253 | 7200 | 3027 | 3056 | 2657 | 2948 |
| 7318 | 7206 | 3027 | 3057 | 2657 | 2949 |
| 7379 | 7277 | 3028 | 3057 | 2658 | 2949 |
| 7486 | 7294 | 3028 | 3058 | 2658 | 2950 |
| 7524 | 7330 | 3029 | 3058 | 2659 | 2950 |
| 7668 | 7411 | 3030 | 3059 | 2659 | 2951 |
| 7680 | 7455 | 3030 | 3059 | 2660 | 2951 |
| 7720 | 7477 | 3031 | 3060 | 2660 | 2952 |
| 1137 | 7511 | 3031 | 3060 | 2661 | 2952 |
| 1138 | 7538 | 3032 | 3061 | 2661 | 2953 |
| 1139 | 7559 | 3032 | 3061 | 2662 | 2953 |
| 1140 | 7560 | 3033 | 3062 | 2662 | 2954 |
| 1141 | 7593 | 3033 | 3062 | 2663 | 2954 |
| 1142 | 7594 | 3034 | 3063 | 2663 | 2955 |
| 1143 | 7607 | 3034 | 3063 | 2664 | 2955 |
| 1144 | 7623 | 6025 | 3064 | 2664 | 6485 |
| 1145 | 7638 | 6041 | 3064 | 2665 | 6486 |
| 1146 | 7717 | 6043 | 3065 | 2665 | 6504 |
| 1147 | 7731 | 6098 | 3065 | 2666 | 6505 |
| 1148 | 7732 | 6099 | 3066 | 2666 | 6595 |
| 1149 | 2711 | 6180 | 3066 | 2667 | 6596 |
| 1150 | 2711 | 6182 | 3067 | 2667 | 6751 |
| 1151 | 2712 | 6237 | 3067 | 2668 | 6752 |
| 1152 | 2712 | 6238 | 3068 | 2668 | 6777 |
| 1153 | 2713 | 6311 | 3068 | 2669 | 6778 |
| 1154 | 2713 | 6312 | 3069 | 2669 | 7172 |
| 1155 | 2714 | 6578 | 3069 | 2670 | 7174 |
| 1156 | 2714 | 6579 | 3070 | 2670 | 7313 |
| 1157 | 2715 | 6663 | 3070 | 2671 | 7314 |
| 1158 | 2715 | 6664 | 3071 | 2671 | |
| 1159 | 2716 | 6708 | 3071 | 2672 | |
| 1160 | 2716 | 6709 | 3072 | 2672 | |
| 1161 | 2717 | 6809 | 3072 | 2673 | |
| 1162 | 2717 | 6831 | 3073 | 2673 | |
| 1163 | 2718 | 6832 | 3073 | 2674 | |
| 1164 | 2718 | 6864 | 3074 | 2674 | |
| 1165 | 2719 | 6866 | 3074 | 2675 | |
| 1166 | 2719 | 7089 | 3075 | 2675 | |
| 1167 | 2720 | 7157 | 3075 | 2676 | |
| 6015 | 2720 | 7159 | 3076 | 2676 | |
| 6029 | 2721 | 7173 | 3076 | 2677 | |
| 6045 | 2721 | 7176 | 3077 | 2677 | |
| 6077 | 2722 | 7293 | 3077 | 2678 | |
| 6129 | 2722 | 7295 | 3078 | 2678 | |
| 6144 | 2723 | 7343 | 3078 | 2679 | |
| 6164 | 2723 | 7393 | 3079 | 2680 | |
| 6201 | 2724 | 7394 | 3079 | 2680 | |
| 6227 | 2724 | 7425 | 3080 | 2681 | |
| 6244 | 2725 | 7426 | 3080 | 2681 | |
| 6250 | 2725 | 7444 | 3081 | 2682 | |
| 6264 | 2726 | 7445 | 3081 | 2682 | |
| 6289 | 2726 | 7476 | 3082 | 2683 | |
| 6304 | 2727 | 7478 | 3082 | 2683 | |
| 6316 | 2727 | 7496 | 3083 | 2684 | |
| 6384 | 2728 | 7497 | 3083 | 2684 | |
| 6421 | 2728 | 7537 | 3084 | 2685 | |
| 6441 | 2729 | 7539 | 3084 | 2685 | |
| 6492 | 2729 | 2780 | 3085 | 2686 | |
| 6514 | 2730 | 2780 | 3085 | 2686 | |
| 6530 | 2730 | 2781 | 3086 | 2687 | |
| 6569 | 2731 | 2781 | 3086 | 2687 | |
| 6584 | 2731 | 2782 | 3087 | 2688 | |
| 6621 | 2732 | 2782 | 3087 | 2688 | |
| 6651 | 2732 | 2783 | 3088 | 2689 | |
| 6659 | 2733 | 2783 | 3088 | 2689 | |
| 6683 | 2734 | 2784 | 3089 | 2690 | |
| 6703 | 2734 | 2784 | 3089 | 2690 | |
| 6727 | 2735 | 2785 | 3090 | 2691 | |
| 6732 | 2735 | 2785 | 3090 | 2692 | |
| 6745 | 2736 | 2786 | 3091 | 2692 | |
| 6755 | 2736 | 2786 | 3091 | 2693 | |
| 6784 | 2737 | 2787 | 3092 | 2693 | |
| 6817 | 2737 | 2787 | 3092 | 2694 | |
| 6823 | 2738 | 2788 | 3093 | 2694 | |
| 6841 | 2739 | 2788 | 3093 | 2695 | |
| 6871 | 2740 | 2789 | 3094 | 2695 | |
| 6885 | 2740 | 2789 | 3095 | 2696 | |
| 6898 | 2741 | 2790 | 3095 | 2696 | |
| 6908 | 2741 | 2790 | 3096 | 2697 | |
| 6933 | 2742 | 2791 | 3096 | 2697 | |
| 6971 | 2742 | 2791 | 3097 | 2698 | |
| 7009 | 2743 | 2792 | 3097 | 2698 | |
| 7018 | 2743 | 2792 | 3098 | 2699 | |
| 7045 | 2744 | 2793 | 3098 | 2699 | |
| 7053 | 2744 | 2793 | 3099 | 2700 | |
| 7068 | 2745 | 2794 | 3099 | 2700 | |
| 7079 | 2745 | 2794 | 3100 | 2701 | |
| 7096 | 2746 | 2795 | 3100 | 2701 | |
| 7104 | 2746 | 2795 | 3101 | 2702 | |
| 7122 | 2747 | 2796 | 3101 | 2702 | |
| 7151 | 2747 | 2796 | 3102 | 2703 | |
| 7163 | 2748 | 2797 | 3102 | 2703 | |
| 7181 | 2748 | 2797 | 3103 | 2862 | |
| 7244 | 2749 | 2798 | 3103 | 2862 | |
| 7273 | 2750 | 2798 | 3104 | 2863 | |
| 7319 | 2750 | 2799 | 3104 | 2863 | |
| 7336 | 2751 | 2799 | 3105 | 2864 | |
| 7380 | 2751 | 2800 | 3105 | 2864 | |
| 7402 | 2752 | 2800 | 3106 | 2865 | |
| 7462 | 2752 | 2801 | 3106 | 2865 | |
| 7487 | 2753 | 2801 | 3107 | 2866 | |
| 7525 | 2753 | 2802 | 3107 | 2866 | |
| 7569 | 2754 | 2802 | 3108 | 2867 | |
| 7626 | 2754 | 2803 | 3108 | 2867 | |
| 7689 | 2755 | 2803 | 3109 | 2868 | |
| 7707 | 2755 | 2804 | 3109 | 2868 | |
| 7721 | 2756 | 2804 | 3110 | 2869 | |
| 1371 | 2756 | 2805 | 3110 | 2869 | |
| 1372 | 2757 | 2805 | 3111 | 2870 | |
| 1373 | 2758 | 2806 | 3111 | 2870 | |
| 1374 | 2758 | 2806 | 3112 | 2871 | |
| 1375 | 2759 | 2807 | 3112 | 2871 | |
| 1376 | 2759 | 2807 | 3113 | 2872 | |
| 1377 | 2760 | 2808 | 3113 | 2872 | |
| 1378 | 2760 | 2808 | 3114 | 2873 | |
| 1379 | 2761 | 2809 | 3114 | 2873 | |
| 1380 | 2761 | 2809 | 3115 | 2874 | |
| 1381 | 2762 | 2810 | 3115 | 2874 | |
| 1382 | 2762 | 2810 | 3116 | 2875 | |
| 1383 | 2763 | 2811 | 3116 | 2875 | |
| 1384 | 2763 | 2811 | 3117 | 2876 | |
| 1385 | 2764 | 2812 | 3117 | 2876 | |
| 1386 | 2764 | 2812 | 3118 | 2877 | |
| 1387 | 2765 | 2813 | 3118 | 2877 | |
| 1388 | 2765 | 2813 | 3119 | 2878 | |
| 1389 | 2766 | 2814 | 3119 | 2878 | |
| 1390 | 2766 | 2814 | 3120 | 2879 | |
| 1391 | 2767 | 2815 | 3120 | 2879 | |
| 1392 | 2767 | 2815 | 3121 | 2880 | |
| 1393 | 2768 | 2816 | 3121 | 2880 | |
| 1394 | 2768 | 2816 | 3122 | 2881 | |
| 1395 | 2769 | 2817 | 3122 | 2881 | |
| 1396 | 2769 | 2817 | 3123 | 2882 | |
| 1397 | 2770 | 2818 | 3123 | 2882 | |
| 1398 | 2770 | 2818 | 3124 | 2883 | |
| 1399 | 2771 | 2819 | 3124 | 2883 | |
| 1400 | 2771 | 2819 | 3125 | 2884 | |
| 1401 | 2772 | 2820 | 3125 | 2884 | |
| 1402 | 2773 | 2820 | 3126 | 2885 | |
| 1403 | 2773 | 2821 | 3126 | 2885 | |
| 1404 | 2774 | 2821 | 3127 | 2886 | |
| 1405 | 2774 | 2822 | 3127 | 2886 | |
| 1406 | 2775 | 2822 | 3128 | 2887 | |
| 1407 | 2775 | 2823 | 3128 | 2887 | |
| 1408 | 2776 | 2823 | 3129 | 2888 | |
| 1409 | 2776 | 2824 | 3129 | 2888 | |
| 1410 | 2777 | 2824 | 3130 | 2889 | |
| 1411 | 2777 | 2825 | 3130 | 2889 | |
| 1412 | 2778 | 2825 | 3131 | 2890 | |
| 1413 | 2779 | 2826 | 3131 | 2890 | |
| 1414 | 2779 | 2826 | 3132 | 2891 | |
| 1415 | 2965 | 2827 | 3133 | 2891 | |
| 1416 | 2965 | 2827 | 3133 | 2892 | |
| 1417 | 2966 | 2828 | 3134 | 2893 | |
| 1418 | 2966 | 2828 | 3134 | 2893 | |
| 1419 | 2967 | 2829 | 3135 | 2894 | |
| 1420 | 2968 | 2829 | 3135 | 2894 | |
| 1421 | 2968 | 2830 | 3136 | 2895 | |
| 1422 | 2969 | 2830 | 3136 | 2895 | |
| 1423 | 2969 | 2831 | 6181 | 2896 | |
| 1424 | 2970 | 2831 | 6183 | 2896 | |
| 1425 | 2970 | 2832 | 6284 | 2897 | |
| 1426 | 2971 | 2832 | 6285 | 2897 | |
| 1427 | 2971 | 2833 | 6760 | 2898 | |
| 1428 | 2972 | 2833 | 6761 | 2898 | |
| 1429 | 2972 | 2834 | 7036 | 2899 | |
| 1430 | 2973 | 2834 | 7038 | 2899 | |
| 1431 | 2974 | 2835 | 7158 | 2900 | |
| 1432 | 2974 | 2835 | 7160 | 2900 | |
| 1433 | 2975 | 2836 | 2610 | 2901 | |
| 1434 | 2976 | 2836 | 2610 | 2901 | |
| 1435 | 2976 | 2837 | 2611 | 2902 | |
| 1436 | 2977 | 2837 | 2611 | 2902 | |
| 1437 | 2977 | 2838 | 2612 | 2903 | |
| 1439 | 2978 | 2838 | 2612 | 2903 | |
| 1440 | 2978 | 2839 | 2613 | 2904 | |
| 1441 | 2979 | 2839 | 2613 | 2904 | |
| 1442 | 2979 | 2840 | 2614 | 2905 | |
| 1443 | 2980 | 2840 | 2614 | 2905 | |
| 1444 | 2980 | 2841 | 2615 | 2906 | |
| 1445 | 2981 | 2841 | 2615 | 2906 | |
| 1446 | 2981 | 2842 | 2616 | 2907 | |
| 1447 | 2982 | 2842 | 2616 | 2907 | |
| 6001 | 2982 | 2843 | 2617 | 2908 | |
| 6030 | 2983 | 2843 | 2617 | 2908 | |
| 6078 | 2983 | 2844 | 2618 | 2909 | |
| 6108 | 2984 | 2844 | 2618 | 2909 | |
| 6130 | 2985 | 2845 | 2619 | 2910 | |
| 6165 | 2985 | 2845 | 2619 | 2910 | |
| 6265 | 2986 | 2846 | 2620 | 2911 | |
| 6275 | 2987 | 2846 | 2620 | 2911 | |
| 6305 | 2987 | 2847 | 2621 | 2912 | |
| 6329 | 2988 | 2847 | 2621 | 2912 | |
| 6370 | 2988 | 2848 | 2622 | 2913 | |
| 6385 | 2989 | 2848 | 2622 | 2913 | |
| 6404 | 2989 | 2849 | 2623 | 2914 | |
| 6531 | 2990 | 2849 | 2623 | 2914 | |
| 6585 | 2990 | 2850 | 2624 | 2915 | |
| 6622 | 2991 | 2850 | 2624 | 2915 | |
| 6652 | 2991 | 2851 | 2625 | 2916 | |
| 6733 | 2992 | 2851 | 2625 | 2916 | |
| 6756 | 2992 | 2852 | 2626 | 2917 | |
| 6765 | 2993 | 2852 | 2626 | 2917 | |
| 6798 | 2993 | 2853 | 2627 | 2918 | |
| 6824 | 2994 | 2853 | 2627 | 2919 | |
| 6972 | 2994 | 2854 | 2628 | 2919 | |
| 7046 | 2995 | 2854 | 2628 | 2920 | |
| 7054 | 2995 | 2855 | 2629 | 2920 | |
| 7069 | 2996 | 2855 | 2629 | 2921 | |
| 7080 | 2996 | 2856 | 2630 | 2921 | |
| 7105 | 2997 | 2856 | 2630 | 2922 | |
| 7123 | 2998 | 2857 | 2631 | 2922 | |
| 7143 | 2998 | 2857 | 2631 | 2923 | |
| 7152 | 2999 | 2858 | 2632 | 2923 | |
| 7204 | 2999 | 2858 | 2632 | 2924 | |
| 7320 | 3001 | 2859 | 2633 | 2924 | |
| 7351 | 3001 | 2859 | 2633 | 2925 | |
| 7381 | 3002 | 2860 | 2634 | 2925 | |
| 7403 | 3002 | 2860 | 2634 | 2926 | |
| 7438 | 3003 | 2861 | 2635 | 2926 | |
| 7488 | 3003 | 2861 | 2635 | 2927 | |
| 7500 | 3004 | 3035 | 2636 | 2927 | |
| 7526 | 3004 | 3036 | 2636 | 2928 | |
| 7588 | 3005 | 3036 | 2637 | 2928 | |
| 7612 | 3005 | 3037 | 2637 | 2929 | |
| 7627 | 3006 | 3037 | 2638 | 2929 | |

### Systems

In an aspect, the disclosure relates to a system comprising nucleic acid molecule encoding a heterologous gene modifying polypeptide (e.g., as described herein) and a template nucleic acid (e.g., a template RNA, e.g., as described herein). In certain embodiments, the nucleic acid molecule encoding the heterologous gene modifying polypeptide comprises one or more silent mutations in the coding region (e.g., in the sequence encoding the RT domain) relative to a nucleic acid molecule as described herein. In certain embodiments, the system further comprises a gRNA (e.g., a gRNA that binds to a polypeptide that induces a nick, e.g., in the opposite strand of the target DNA bound by the heterologous gene modifying polypeptide).

In certain embodiments, the nucleic acid molecule encoding the heterologous gene modifying polypeptide encodes a polypeptide having an amino acid sequence selected from SEQ ID NOs: 1-7743, or an amino acid sequence having at least 70%, 75%, 80%, 85%, 90%, 95%, or 99% identity thereto. In certain embodiments, the nucleic acid molecule encoding the heterologous gene modifying polypeptide encodes a polypeptide having an amino acid sequence selected from SEQ ID NOs: 6001-7743, or an amino acid sequence having at least 70%, 75%, 80%, 85%, 90%, 95%, or 99% identity thereto. In certain embodiments, the nucleic acid molecule encoding the heterologous gene modifying polypeptide encodes a polypeptide having an amino acid sequence selected from SEQ ID NOs: 4501-4541, or an amino acid sequence having at least 70%, 75%, 80%, 85%, 90%, 95%, or 99% identity thereto. In certain embodiments, the nucleic acid molecule encoding the heterologous gene modifying polypeptide encodes a polypeptide as listed in any of Tables A1, T1, or T2, or an amino acid sequence having at least 70%, 75%, 80%, 85%, 90%, 95%, or 99% identity thereto.

In certain embodiments, the nucleic acid molecule encoding the heterologous gene modifying polypeptide comprises a sequence encoding a portion of an amino acid sequence selected from SEQ ID NOs: 1-7743, wherein the portion comprises a linker and RT domain, or an amino acid sequence having at least 70%, 75%, 80%, 85%, 90%, 95%, or 99% identity to said portion. In certain embodiments, the nucleic acid molecule encoding the heterologous gene modifying polypeptide comprises a sequence encoding a portion of an amino acid sequence selected from SEQ ID NOs: 6001-7743, wherein the portion comprises a linker and RT domain, or an amino acid sequence having at least 70%, 75%, 80%, 85%, 90%, 95%, or 99% identity to said portion. In certain embodiments, the nucleic acid molecule encoding the heterologous gene modifying polypeptide comprises a sequence encoding a portion of an amino acid sequence selected from SEQ ID NOs: 4501-4541, wherein the portion comprises a linker and RT domain, or an amino acid sequence having at least 70%, 75%, 80%, 85%, 90%, 95%, or 99% identity to said portion. In certain embodiments, the nucleic acid molecule encoding the heterologous gene modifying polypeptide comprises a sequence encoding a portion of a polypeptide listed in any of Tables A1, T1, or T2, wherein the portion comprises a linker and RT domain, or an amino acid sequence having at least 70%, 75%, 80%, 85%, 90%, 95%, or 99% identity to said portion.

In certain embodiments, the nucleic acid molecule encoding the heterologous gene modifying polypeptide comprises a sequence encoding the linker of an amino acid sequence selected from SEQ ID NOs: 1-7743, or an amino acid sequence having at least 70%, 75%, 80%, 85%, 90%, 95%, or 99% identity thereto. In certain embodiments, the nucleic acid molecule encoding the heterologous gene modifying polypeptide comprises a sequence encoding the linker of a polypeptide having an amino acid sequence selected from SEQ ID NOs: 6001-7743, or an amino acid sequence having at least 70%, 75%, 80%, 85%, 90%, 95%, or 99% identity thereto. In certain embodiments, the nucleic acid molecule encoding the heterologous gene modifying polypeptide comprises a sequence encoding the linker of a polypeptide having an amino acid sequence selected from SEQ ID NOs: 4501-4541, or an amino acid sequence having at least 70%, 75%, 80%, 85%, 90%, 95%, or 99% identity thereto. In certain embodiments, the nucleic acid molecule encoding the heterologous gene modifying polypeptide comprises a sequence encoding the linker of a polypeptide as listed in any of Tables A1, T1, or T2, or an amino acid sequence having at least 70%, 75%, 80%, 85%, 90%, 95%, or 99% identity thereto.

In certain embodiments, the nucleic acid molecule encoding the heterologous gene modifying polypeptide comprises a sequence encoding the RT domain of an amino acid sequence selected from SEQ ID NOs: 1-7743, or an amino acid sequence having at least 70%, 75%, 80%, 85%, 90%, 95%, or 99% identity thereto. In certain embodiments, the nucleic acid molecule encoding the heterologous gene modifying polypeptide comprises a sequence encoding the RT domain of a polypeptide having an amino acid sequence selected from SEQ ID NOs: 6001-7743, or an amino acid sequence having at least 70%, 75%, 80%, 85%, 90%, 95%, or 99% identity thereto. In certain embodiments, the nucleic acid molecule encoding the heterologous gene modifying polypeptide comprises a sequence encoding the RT domain of a polypeptide having an amino acid sequence selected from SEQ ID NOs: 4501-4541, or an amino acid sequence having at least 70%, 75%, 80%, 85%, 90%, 95%, or 99% identity thereto. In certain embodiments, the nucleic acid molecule encoding the heterologous gene modifying polypeptide comprises a sequence encoding the RT domain of a polypeptide as listed in any of Tables A1, T1, or T2, or an amino acid sequence having at least 70%, 75%, 80%, 85%, 90%, 95%, or 99% identity thereto.

In an aspect, the disclosure relates to a system comprising a heterologous gene modifying polypeptide (e.g., as described herein) and a template nucleic acid (e.g., a template RNA, e.g., as described herein).

In certain embodiments, the heterologous gene modifying polypeptide comprises a polypeptide having an amino acid sequence selected from SEQ ID NOs: 1-7743, or an amino acid sequence having at least 70%, 75%, 80%, 85%, 90%, 95%, or 99% identity thereto. In certain embodiments, the heterologous gene modifying polypeptide comprises a polypeptide having an amino acid sequence selected from SEQ ID NOs: 6001-7743, or an amino acid sequence having at least 70%, 75%, 80%, 85%, 90%, 95%, or 99% identity thereto. In certain embodiments, the heterologous gene modifying polypeptide comprises a polypeptide having an amino acid sequence selected from SEQ ID NOs: 4501-4541, or an amino acid sequence having at least 70%, 75%, 80%, 85%, 90%, 95%, or 99% identity thereto. In certain embodiments, the heterologous gene modifying polypeptide comprises a polypeptide as listed in any of Tables A1, T1, or T2, or an amino acid sequence having at least 70%, 75%, 80%, 85%, 90%, 95%, or 99% identity thereto.

In certain embodiments, the heterologous gene modifying polypeptide comprises a portion of an amino acid sequence selected from SEQ ID NOs: 1-7743, wherein the portion comprises a linker and RT domain, or an amino acid sequence having at least 70%, 75%, 80%, 85%, 90%, 95%, or 99% identity to said portion. In certain embodiments, the heterologous gene modifying polypeptide comprises a portion of an amino acid sequence selected from SEQ ID NOs: 6001-7743, wherein the portion comprises a linker and RT domain, or an amino acid sequence having at least 70%, 75%, 80%, 85%, 90%, 95%, or 99% identity to said portion. In certain embodiments, the heterologous gene modifying polypeptide comprises a portion of an amino acid sequence selected from SEQ ID NOs: 4501-4541, wherein the portion comprises a linker and RT domain, or an amino acid sequence having at least 70%, 75%, 80%, 85%, 90%, 95%, or 99% identity to said portion. In certain embodiments, the heterologous gene modifying polypeptide comprises a portion of a polypeptide listed in any of Tables A1, T1, or T2, wherein the portion comprises a linker and RT domain, or an amino acid sequence having at least 70%, 75%, 80%, 85%, 90%, 95%, or 99% identity to said portion.

In certain embodiments, the heterologous gene modifying polypeptide comprises the linker of an amino acid sequence selected from SEQ ID NOs: 1-7743, or an amino acid sequence having at least 70%, 75%, 80%, 85%, 90%, 95%, or 99% identity thereto. In certain embodiments, the heterologous gene modifying polypeptide comprises a sequence encoding the linker of a polypeptide having an amino acid sequence selected from SEQ ID NOs: 6001-7743, or an amino acid sequence having at least 70%, 75%, 80%, 85%, 90%, 95%, or 99% identity thereto. In certain embodiments, the heterologous gene modifying polypeptide comprises a sequence encoding the linker of a polypeptide having an amino acid sequence selected from SEQ ID NOs: 4501-4541, or an amino acid sequence having at least 70%, 75%, 80%, 85%, 90%, 95%, or 99% identity thereto. In certain embodiments, the heterologous gene modifying polypeptide comprises the linker of a polypeptide as listed in any of Tables A1, T1, or T2, or an amino acid sequence having at least 70%, 75%, 80%, 85%, 90%, 95%, or 99% identity thereto.

In certain embodiments, the heterologous gene modifying polypeptide comprises the RT domain of an amino acid sequence selected from SEQ ID NOs: 1-7743, or an amino acid sequence having at least 70%, 75%, 80%, 85%, 90%, 95%, or 99% identity thereto. In certain embodiments, the heterologous gene modifying polypeptide comprises a sequence encoding the RT domain of a polypeptide having an amino acid sequence selected from SEQ ID NOs: 6001-7743, or an amino acid sequence having at least 70%, 75%, 80%, 85%, 90%, 95%, or 99% identity thereto. In certain embodiments, the heterologous gene modifying polypeptide comprises a sequence encoding the RT domain of a polypeptide having an amino acid sequence selected from SEQ ID NOs: 4501-4541, or an amino acid sequence having at least 70%, 75%, 80%, 85%, 90%, 95%, or 99% identity thereto. In certain embodiments, the heterologous gene modifying polypeptide comprises the RT domain of a polypeptide as listed in any of Tables A1, T1, or T2, or an amino acid sequence having at least 70%, 75%, 80%, 85%, 90%, 95%, or 99% identity thereto.

Lengthy table referenced here
US20260224743A1-20260806-T00001
Please refer to the end of the specification for access instructions.

### Localization Sequences for Gene Modifying Systems

In certain embodiments, a gene editor system RNA further comprises an intracellular localization sequence, e.g., a nuclear localization sequence (NLS). In some embodiments, a heterologous gene modifying polypeptide comprises an NLS as comprised in SEQ ID NO: 4000 and/or SEQ ID NO: 4001, or an NLS having an amino acid sequence having at least 75%, 80%, 85%, 90%, 95%, 96%, 97%, 98%, or 99% identity thereto.

The nuclear localization sequence may be an RNA sequence that promotes the import of the RNA into the nucleus. In certain embodiments the nuclear localization signal is located on the template RNA. In certain embodiments, the heterologous gene modifying polypeptide is encoded on a first RNA, and the template RNA is a second, separate, RNA, and the nuclear localization signal is located on the template RNA and not on an RNA encoding the heterologous gene modifying polypeptide. While not wishing to be bound by theory, in some embodiments, the RNA encoding the heterologous gene modifying polypeptide is targeted primarily to the cytoplasm to promote its translation, while the template RNA is targeted primarily to the nucleus to promote insertion into the genome. In some embodiments the nuclear localization signal is at the 3′ end, 5′ end, or in an internal region of the template RNA. In some embodiments the nuclear localization signal is 3′ of the heterologous sequence (e.g., is directly 3′ of the heterologous sequence) or is 5′ of the heterologous sequence (e.g., is directly 5′ of the heterologous sequence). In some embodiments the nuclear localization signal is placed outside of the 5′ UTR or outside of the 3′ UTR of the template RNA. In some embodiments the nuclear localization signal is placed between the 5′ UTR and the 3′ UTR, wherein optionally the nuclear localization signal is not transcribed with the transgene (e.g., the nuclear localization signal is an anti-sense orientation or is downstream of a transcriptional termination signal or polyadenylation signal). In some embodiments the nuclear localization sequence is situated inside of an intron. In some embodiments a plurality of the same or different nuclear localization signals are in the RNA, e.g., in the template RNA. In some embodiments the nuclear localization signal is less than 5, 10, 25, 50, 75, 100, 150, 200, 250, 300, 350, 400, 450, 500, 600, 700, 800, 900 or 1000 bp in length. Various RNA nuclear localization sequences can be used. For example, Lubelsky and Ulitsky, Nature 555 (107-111), 2018 describe RNA sequences which drive RNA localization into the nucleus. In some embodiments, the nuclear localization signal is a SINE-derived nuclear RNA localization (SIRLOIN) signal. In some embodiments the nuclear localization signal binds a nuclear-enriched protein. In some embodiments the nuclear localization signal binds the HNRNPK protein. In some embodiments the nuclear localization signal is rich in pyrimidines, e.g., is a C/T rich, C/U rich, C rich, T rich, or U rich region. In some embodiments the nuclear localization signal is derived from a long non-coding RNA. In some embodiments the nuclear localization signal is derived from MALATI long non-coding RNA or is the 600 nucleotide M region of MALATI (described in Miyagawa et al., RNA 18, (738-751), 2012). In some embodiments the nuclear localization signal is derived from BORG long non-coding RNA or is a AGCCC motif (described in Zhang et al., Molecular and Cellular Biology 34, 2318-2329 (2014). In some embodiments the nuclear localization sequence is described in Shukla et al., The EMBO Journal e98452 (2018). In some embodiments the nuclear localization signal is derived from a retrovirus.

In some embodiments, a polypeptide described herein comprises one or more (e.g., 2, 3, 4, 5) nuclear targeting sequences, for example a nuclear localization sequence (NLS). In some embodiments, the NLS is a bipartite NLS. In some embodiments, an NLS facilitates the import of a protein comprising an NLS into the cell nucleus. In some embodiments, the NLS is fused to the N-terminus of a heterologous gene modifying polypeptide as described herein. In some embodiments, the NLS is fused to the C-terminus of the heterologous gene modifying polypeptide. In some embodiments, the NLS is fused to the N-terminus or the C-terminus of a Cas domain. In some embodiments, a linker sequence is disposed between the NLS and the neighboring domain of the heterologous gene modifying polypeptide.

In some embodiments, an NLS comprises the amino acid sequence MDSLLMNRRKFLYQFKNVRWAKGRRETYLC (SEQ ID NO: 5009), PKKRKVEGADKRTADGSEFESPKKKRKV (SEQ ID NO: 5010), RKSGKIAAIWKRPRKPKKKRKV (SEQ ID NO: 5011) KRTADGSEFESPKKKRKV (SEQ ID NO: 5012), KKTELQTTNAENKTKKL (SEQ ID NO: 5013), or KRGINDRNFWRGENGRKTR (SEQ ID NO: 5014), KRPAATKKAGQAKKKK (SEQ ID NO: 5015), PAAKRVKLD (SEQ ID NO: 4644), KRTADGSEFEKRTADGSEFESPKKKAKVE (SEQ ID NO: 4649), KRTADGSEFE (SEQ ID NO: 4650), KRTADGSEFESPKKKAKVE (SEQ ID NO: 11098), AGKRTADGSEFEKRTADGSEFESPKKKAKVE (SEQ ID NO: 4001), or a functional fragment or variant thereof. Exemplary NLS sequences are also described in PCT/EP2000/011690, the contents of which are incorporated herein by reference for their disclosure of exemplary nuclear localization sequences. In some embodiments, an NLS comprises an amino acid sequence as disclosed in Table 11. An NLS of this table may be utilized with one or more copies in a polypeptide in one or more locations in a polypeptide, e.g., 1, 2, 3 or more copies of an NLS in an N-terminal domain, between peptide domains, in a C-terminal domain, or in a combination of locations, in order to improve subcellular localization to the nucleus. Multiple unique sequences may be used within a single polypeptide. Sequences may be naturally monopartite or bipartite, e.g., having one or two stretches of basic amino acids, or may be used as chimeric bipartite sequences. Sequence references correspond to UniProt accession numbers, except where indicated as SeqNLS for sequences mined using a subcellular localization prediction algorithm (Lin et al BMC Bioinformat 13:157 (2012), incorporated herein by reference in its entirety).

TABLE 11
Exemplary nuclear localization signals for use in heterologous gene
modifying systems

| Sequence | Sequence References | SEQ ID No. |
|---|---|---|
| AHFKISGEKRPSTDPGKKAK | Q76IQ7 | 5223 |
| NPKKKKKKDP | | |
| AHRAKKMSKTHA | P21827 | 5224 |
| ASPEYVNLPINGNG | SeqNLS | 5225 |
| CTKRPRW | O88622, Q86W56, Q9QYM2, O02776 | 5226 |
| DKAKRVSRNKSEKKRR | O15516, Q5RAK8, Q91YB2, Q91YB0, | 5227 |
| | Q8QGQ6, O08785, Q9WVS9, Q6YGZ4 | |
| EELRLKEELLKGIYA | Q9QY16, Q9UHL0, Q2TBP1, Q9QY15 | 5228 |
| EEQLRRRKNSRLNNTG | G5EFF5 | 5229 |
| EVLKVIRTGKRKKKAWKR | SeqNLS | 5230 |
| MVTKVC | | |
| HHHHHHHHHHHHQPH | Q63934, G3V7L5, Q12837 | 5231 |
| HKKKHPDASVNFSEFSK | P10103, Q4R844, P12682, B0CM99, | 5232 |
| | A9RA84, Q6YKA4, P09429, P63159, | |
| | Q08IE6, P63158, Q9YH06, B1MTB0 | |
| HKRTKK | Q2R2D5 | 5233 |
| IINGRKLKLKKSRRRSSQTS | SeqNLS | 5234 |
| NNSFTSRRS | | |
| KAEQERRK | Q8LH59 | 5235 |
| KEKRKRREELFIEQKKRK | SeqNLS | 5236 |
| KKGKDEWFSRGKKP | P30999 | 5237 |
| KKGPSVQKRKKT | Q6ZN17 | 5238 |
| KKKTVINDLLHYKKEK | SeqNLS, P32354 | 5239 |
| KKNGGKGKNKPSAKIKK | SeqNLS | 5240 |
| KKPKWDDFKKKKK | Q15397, Q8BKS9, Q562C7 | 5241 |
| KKRKKD | SeqNLS, Q91Z62, Q1A730, Q969P5, | 5242 |
| | Q2KHT6, Q9CPU7 | |
| KKRRKRRRK | SeqNLS | 5243 |
| KKRRRRARK | Q9UMS6, D4A702, Q91YE8 | 5244 |
| KKSKRGR | Q9UBS0 | 5245 |
| KKSRKRGS | B4FG96 | 5246 |
| KKSTALSRELGKIMRRR | SeqNLS, P32354 | 5247 |
| KKSYQDPEIIAHSRPRK | Q9U7C9 | 5248 |
| KKTGKNRKLKSKRVKTR | Q9Z301, O54943, Q8K3T2 | 5249 |
| KKVSIAGQSGKLWRWKR | Q6YUL8 | 5250 |
| KKYENVVIKRSPRKRGRPR | SeqNLS | 5251 |
| K | | |
| KNKKRK | SeqNLS | 5252 |
| KPKKKR | SeqNLS | 5253 |
| KRAMKDDSHGNSTSPKRRK | Q0E671 | 5254 |
| KRANSNLVAAYEKAKKK | P23508 | 5255 |
| KRASEDTTSGSPPKKSSAGP | Q9BZZ5, Q5R644 | 5256 |
| KR | | |
| KRFKRRWMVRKMKTKK | SeqNLS | 5257 |
| KRGLNSSFETSPKKVK | Q8IV63 | 5258 |
| KRGNSSIGPNDLSKRKQRK | SeqNLS | 5259 |
| K | | |
| KRIHSVSLSQSQIDPSKKVK | SeqNLS | 5260 |
| RAK | | |
| KRKGKLKNKGSKRKK | O15381 | 5261 |
| KRRRRRRREKRKR | Q96GM8 | 5262 |
| KRSNDRTYSPEEEKQRRA | Q91ZF2 | 5263 |
| KRTVATNGDASGAHRAKK | SeqNLS | 5264 |
| MSK | | |
| KRVYNKGEDEQEHLPKGKK | SeqNLS | 5265 |
| R | | |
| KSGKAPRRRAVSMDNSNK | Q9WVH4, O43524 | 5266 |
| KVNFLDMSLDDIIIYKELE | Q9P127 | 5267 |
| KVQHRIAKKTTRRRR | Q9DXE6 | 5268 |
| LSPSLSPL | Q9Y261, P32182, P35583 | 5269 |
| MDSLLMNRRKFLYQFKNVR | Q9GZX7 | 5270 |
| WAKGRRETYLC | | |
| MPQNEYIELHRKRYGYRLD | SeqNLS | 5271 |
| YHEKKRKKESREAHERSKK | | |
| AKKMIGLKAKLYHK | | |
| MVQLRPRASR | SeqNLS | 5272 |
| NNKLLAKRRKGGASPKDDP | Q965G5 | 5273 |
| MDDIK | | |
| NYKRPMDGTYGPPAKRHEG | O14497, A2BH40 | 5274 |
| E | | |
| PDTKRAKLDSSETTMVKKK | SeqNLS | 5275 |
| PEKRTKI | SeqNLS | 5276 |
| PGGRGKKK | Q719N1, Q9UBP0, A2VDN5 | 5277 |
| PGKMDKGEHRQERRDRPY | Q01844, Q61545 | 5278 |
| PKKGDKYDKTD | Q45FA5 | 5279 |
| PKKKSRK | O35914, Q01954 | 5280 |
| PKKNKPE | Q22663 | 5281 |
| PKKRAKV | P04295, P89438 | 5282 |
| PKPKKLKVE | P55263, P55262, P55264, Q64640 | 5283 |
| PKRGRGR | Q9FYS5, Q43386 | 5284 |
| PKRRLVDDA | P0C797 | 5285 |
| PKRRRTY | SeqNLS | 5286 |
| PLFKRR | A8X6H4, Q9TXJ0 | 5287 |
| PLRKAKR | Q86WB0, Q5R8V9 | 5288 |
| PPAKRKCIF | Q6AZ28, O75928, Q8C5D8 | 5289 |
| PPARRRRL | Q8NAG6 | 5290 |
| PPKKKRKV | Q3L6L5, P03070, P14999, P03071 | 5291 |
| PPNKRMKVKH | Q8BN78 | 5292 |
| PPRIYPQLPSAPT | P0C799 | 5293 |
| PQRSPFPKSSVKR | SeqNLS | 5294 |
| PRPRKVPR | P0C799 | 5295 |
| PRRRVQRKR | SeqNLS, Q5R448, Q5TAQ9 | 5296 |
| PRRVRLK | Q58DJ0, P56477, Q13568 | 5297 |
| PSRKRPR | Q62315, Q5F363, Q92833 | 5298 |
| PSSKKRKV | SeqNLS | 5299 |
| PTKKRVK | P07664 | 5300 |
| QRPGPYDRP | SeqNLS | 5301 |
| RGKGGKGLGKGGAKRHRK | SeqNLS | 5302 |
| RKAGKGGGGHKTTKKRSA | B4FG96 | 5303 |
| KDEKVP | | |
| RKIKLKRAK | A1L3G9 | 5304 |
| RKIKRKRAK | B9X187 | 5305 |
| RKKEAPGPREELRSRGR | O35126, P54258, Q5IS70, P54259 | 5306 |
| RKKRKGK | SeqNLS, Q29243, Q62165, Q28685, | 5307 |
| | O18738, Q9TSZ6, Q14118 | |
| RKKRRQRRR | P04326, P69697, P69698, P05907, | 5308 |
| | P20879, P04613, P19553, P0C1J9, | |
| | P20893, P12506, P04612, Q73370, | |
| | P0C1K0, P05906, P35965, P04609, | |
| | P04610, P04614, P04608, P05905 | |
| RKKSIPLSIKNLKRKHKRKK | Q9C0C9 | 5309 |
| NKITR | | |
| RKLVKPKNTKMKTKLRTNP | Q14190 | 5310 |
| Y | | |
| RKRLILSDKGQLDWKK | SeqNLS, Q91Z62, Q1A730, Q2KHT6, | 5311 |
| | Q9CPU7 | |
| RKRLKSK | Q13309 | 5312 |
| RKRRVRDNM | Q8QPH4, Q809M7, A8C8X1, Q2VNC5, | 5313 |
| | Q38SQ0, O89749, Q6DNQ9, Q809L9, | |
| | Q0A429, Q20NV3, P16509, P16505, | |
| | Q6DNQ5, P16506, Q6XT06, P26118, | |
| | Q2ICQ2, Q2RCG8, Q0A2DO, Q0A2H9, | |
| | Q9IQ46, Q809M3, Q6J847, Q6J856, | |
| | B4URE4, A4GCM7, Q0A440, P26120, | |
| | P16511, | |
| RKRSPKDKKEKDLDGAGKR | Q7RTP6 | 5314 |
| RKT | | |
| RKRTPRVDGQTGENDMNK | O94851 | 5315 |
| RRRK | | |
| RLPVRRRRRR | P04499, P12541, P03269, P48313, | 5316 |
| | P03270 | |
| RLRFRKPKSK | P69469 | 5317 |
| RQQRKR | Q14980 | 5318 |
| RRDLNSSFETSPKKVK | Q8K3G5 | 5319 |
| RRDRAKLR | Q9SLB8 | 5320 |
| RRGDGRRR | Q80WE1, Q5R9B4, Q06787, P35922 | 5321 |
| RRGRKRKAEKQ | Q812D1, Q5XXA9, Q99JF8, Q8MJG1, | 5322 |
| | Q66T72, O75475 | |
| RRKKRR | Q0VD86, Q58DS6, Q5R6G2, Q9ERI5, | 5323 |
| | Q6AYK2, Q6NYC1 | |
| RRKRSKSEDMDSVESKRRR | Q7TT18 | 5324 |
| RRKRSR | Q99PU7, D3ZHS6, Q92560, A2VDM8 | 5325 |
| RRPKGKTLQKRKPK | Q6ZN17 | 5326 |
| RRRGFERFGPDNMGRKRK | Q63014, Q9DBR0 | 5327 |
| RRRGKNKVAAQNCRK | SeqNLS | 5328 |
| RRRKRR | Q5FVH8, Q6MZT1, Q08DH5, Q8BQP9 | 5329 |
| RRRQKQKGGASRRR | SeqNLS | 5330 |
| RRRREGPRARRRR | P08313, P10231 | 5331 |
| RRTIRLKLVYDKCDRSCKIQ | SeqNLS | 5332 |
| KKNRNKCQYCRFHKCLSVG | | |
| MSHNAIRFGRMPRSEKAKL | | |
| KAE | | |
| RRVPQRKEVSRCRKCRK | Q5RJN4, Q32L09, Q8CAK3, Q9NUL5 | 5333 |
| RVGGRRQAVECIEDLLNEP | P03255 | 5334 |
| GQPLDLSCKRPRP | | |
| RVVKLRIAP | P52639, Q8JMN0 | 5335 |
| RVVRRR | P70278 | 5336 |
| SKRKTKISRKTR | Q5RAY1, O00443 | 5337 |
| SYVKTVPNRTRTYIKL | P21935 | 5338 |
| TGKNEAKKRKIA | P52739, Q8K3J5, Q5RAU9 | 5339 |
| TLSPASSPSSVSCPVIPASTD | SeqNLS | 5340 |
| ESPGSALNI | | |
| VSKKQRTGKKIH | P52739, Q8K3J5, Q5RAU9 | 5341 |
| SPKKKRKVE | | 5342 |
| KRTAD GSEFE SPKKKRKVE | | 5343 |
| PAAKRVKLD | | 5344 |
| PKKKRKV | | 5345 |
| MDSLLMNRRKFLYQFKNVR | | 5346 |
| WAKGRRETYLC | | |
| SPKKKRKVEAS | | 5347 |
| MAPKKKRKVGIHRGVP | | 5348 |
| KRTADGSEFEKRTADGSEFE | | 5349 |
| SPKKKAKVE | | |
| KRTADGSEFE | | 5350 |
| KRTADGSEFESPKKKAKVE | | 5351 |
| AGKRTADGSEFEKRTADGS | | 4001 |
| EFESPKKKAKVE | | |

In some embodiments, the NLS is a bipartite NLS. A bipartite NLS typically comprises two basic amino acid clusters separated by a spacer sequence (which may be, e.g., about 10 amino acids in length). A monopartite NLS typically lacks a spacer. An example of a bipartite NLS is the nucleoplasmin NLS, having the sequence KR [PAATKKAGQA]KKKK (SEQ ID NO: 5015), wherein the spacer is bracketed. Another exemplary bipartite NLS has the sequence PKKKRKVEGADKRTADGSEFESPKKKRKV (SEQ ID NO: 5016). Exemplary NLSs are described in International Application WO2020051561, which is herein incorporated by reference in its entirety, including for its disclosures regarding nuclear localization sequences.

In certain embodiments, a gene editor system polypeptide (e.g., a heterologous gene modifying polypeptide as described herein) further comprises an intracellular localization sequence, e.g., a nuclear localization sequence and/or a nucleolar localization sequence. The nuclear localization sequence and/or nucleolar localization sequence may be amino acid sequences that promote the import of the protein into the nucleus and/or nucleolus, where it can promote integration of heterologous sequence into the genome. In certain embodiments, a gene editor system polypeptide (e.g., (e.g., a heterologous gene modifying polypeptide as described herein) further comprises a nucleolar localization sequence. In certain embodiments, the heterologous gene modifying polypeptide is encoded on a first RNA, and the template RNA is a second, separate, RNA, and the nucleolar localization signal is encoded on the RNA encoding the heterologous gene modifying polypeptide and not on the template RNA. In some embodiments, the nucleolar localization signal is located at the N-terminus, C-terminus, or in an internal region of the polypeptide. In some embodiments, a plurality of the same or different nucleolar localization signals are used. In some embodiments, the nuclear localization signal is less than 5, 10, 25, 50, 75, or 100 amino acids in length. Various polypeptide nucleolar localization signals can be used. For example, Yang et al., Journal of Biomedical Science 22, 33 (2015), describe a nuclear localization signal that also functions as a nucleolar localization signal. In some embodiments, the nucleolar localization signal may also be a nuclear localization signal. In some embodiments, the nucleolar localization signal may overlap with a nuclear localization signal. In some embodiments, the nucleolar localization signal may comprise a stretch of basic residues. In some embodiments, the nucleolar localization signal may be rich in arginine and lysine residues. In some embodiments, the nucleolar localization signal may be derived from a protein that is enriched in the nucleolus. In some embodiments, the nucleolar localization signal may be derived from a protein enriched at ribosomal RNA loci. In some embodiments, the nucleolar localization signal may be derived from a protein that binds rRNA. In some embodiments, the nucleolar localization signal may be derived from MSP58. In some embodiments, the nucleolar localization signal may be a monopartite motif. In some embodiments, the nucleolar localization signal may be a bipartite motif. In some embodiments, the nucleolar localization signal may consist of a multiple monopartite or bipartite motifs. In some embodiments, the nucleolar localization signal may consist of a mix of monopartite and bipartite motifs. In some embodiments, the nucleolar localization signal may be a dual bipartite motif. In some embodiments, the nucleolar localization motif may be a KRASSQALGTIPKRRSSSRFIKRKK (SEQ ID NO: 5017). In some embodiments, the nucleolar localization signal may be derived from nuclear factor-KB-inducing kinase. In some embodiments, the nucleolar localization signal may be an RKKRKKK motif (SEQ ID NO: 5018) (described in Birbach et al., Journal of Cell Science, 117 (3615-3624), 2004).

### Evolved Variants of Heterologous Gene Modifying Polypeptides and Systems

In some embodiments, the invention provides evolved variants of heterologous gene modifying polypeptides as described herein. Evolved variants can, in some embodiments, be produced by mutagenizing a reference heterologous gene modifying polypeptide, or one of the fragments or domains comprised therein. In some embodiments, one or more of the domains (e.g., the reverse transcriptase domain) is evolved. One or more of such evolved variant domains can, in some embodiments, be evolved alone or together with other domains. An evolved variant domain or domains may, in some embodiments, be combined with unevolved cognate component(s) or evolved variants of the cognate component(s), e.g., which may have been evolved in either a parallel or serial manner.

In some embodiments, the process of mutagenizing a reference heterologous gene modifying polypeptide, or fragment or domain thereof, comprises mutagenizing the reference heterologous gene modifying polypeptide or fragment or domain thereof. In embodiments, the mutagenesis comprises a continuous evolution method (e.g., PACE) or non-continuous evolution method (e.g., PANCE), e.g., as described herein. In some embodiments, the evolved heterologous gene modifying polypeptide, or a fragment or domain thereof, comprises one or more amino acid variations introduced into its amino acid sequence relative to the amino acid sequence of the reference heterologous gene modifying polypeptide, or fragment or domain thereof. In embodiments, amino acid sequence variations may include one or more mutated residues (e.g., conservative substitutions, non-conservative substitutions, or a combination thereof) within the amino acid sequence of a reference heterologous gene modifying polypeptide, e.g., as a result of a change in the nucleotide sequence encoding the heterologous gene modifying polypeptide that results in, e.g., a change in the codon at any particular position in the coding sequence, the deletion of one or more amino acids (e.g., a truncated protein), the insertion of one or more amino acids, or any combination of the foregoing. The evolved variant heterologous gene modifying polypeptide may include variants in one or more components or domains of the heterologous gene modifying polypeptide (e.g., variants introduced into a reverse transcriptase domain).

In some aspects, the disclosure provides heterologous gene modifying polypeptides, systems, kits, and methods using or comprising an evolved variant of a heterologous gene modifying polypeptide, e.g., employs an evolved variant of a heterologous gene modifying polypeptide or a heterologous gene modifying polypeptide produced or producible by PACE or PANCE. In embodiments, the unevolved reference heterologous gene modifying polypeptide is a heterologous gene modifying polypeptide as disclosed herein.

The term “phage-assisted continuous evolution (PACE),” as used herein, generally refers to continuous evolution that employs phage as viral vectors. Examples of PACE technology have been described, for example, in International PCT Application No. PCT/US 2009/056194, filed Sep. 8, 2009, published as WO 2010/028347 on Mar. 11, 2010; International PCT application, PCT/US2011/066747, filed Dec. 22, 2011, published as WO 2012/088381 on Jun. 28, 2012; U.S. Pat. No. 9,023,594, issued May 5, 2015; U.S. Pat. No. 9,771,574, issued Sep. 26, 2017; U.S. Pat. No. 9,394,537, issued Jul. 19, 2016; International PCT application, PCT/US2015/012022, filed Jan. 20, 2015, published as WO 2015/134121 on Sep. 11, 2015; U.S. Pat. No. 10,179,911, issued Jan. 15, 2019; and International PCT application, PCT/US2016/027795, filed Apr. 15, 2016, published as WO 2016/168631 on Oct. 20, 2016, the entire contents of each of which are incorporated herein by reference.

The term “phage-assisted non-continuous evolution (PANCE),” as used herein, generally refers to non-continuous evolution that employs phage as viral vectors. Examples of PANCE technology have been described, for example, in Suzuki T. et al, Crystal structures reveal an elusive functional domain of pyrrolysyl-tRNA synthetase, Nat Chem Biol. 13 (12): 1261-1266 (2017), incorporated herein by reference in its entirety. Briefly, PANCE is a technique for rapid in vivo directed evolution using serial flask transfers of evolving selection phage (SP), which contain a gene of interest to be evolved, across fresh host cells (e.g., E. coli cells). Genes inside the host cell may be held constant while genes contained in the SP continuously evolve. Following phage growth, an aliquot of infected cells may be used to transfect a subsequent flask containing host E. coli. This process can be repeated and/or continued until the desired phenotype is evolved, e.g., for as many transfers as desired.

Methods of applying PACE and PANCE to heterologous gene modifying polypeptides may be readily appreciated by the skilled artisan by reference to, inter alia, the foregoing references. Additional exemplary methods for directing continuous evolution of genome-modifying proteins or systems, e.g., in a population of host cells, e.g., using phage particles, can be applied to generate evolved variants of heterologous gene modifying polypeptides, or fragments or subdomains thereof. Non-limiting examples of such methods are described in International PCT application, PCT/US2009/056194, filed Sep. 8, 2009, published as WO 2010/028347 on Mar. 11, 2010; International PCT application, PCT/US2011/066747, filed Dec. 22, 2011, published as WO 2012/088381 on Jun. 28, 2012; U.S. Pat. No. 9,023,594, issued May 5, 2015; U.S. Pat. No. 9,771,574, issued Sep. 26, 2017; U.S. Pat. No. 9,394,537, issued Jul. 19, 2016; International PCT application, PCT/US2015/012022, filed Jan. 20, 2015, published as WO 2015/134121 on Sep. 11, 2015; U.S. Pat. No. 10,179,911, issued Jan. 15, 2019; International Application No. PCT/US2019/37216, filed Jun. 14, 2019, International Patent Publication WO 2019/023680, published Jan. 31, 2019, International PCT application, PCT/US2016/027795, filed Apr. 15, 2016, published as WO 2016/168631 on Oct. 20, 2016, and International Patent Publication No. PCT/US2019/47996, filed Aug. 23, 2019, each of which is incorporated herein by reference in its entirety.

In some non-limiting illustrative embodiments, a method of evolution of a evolved variant heterologous gene modifying polypeptide, of a fragment or domain thereof, comprises: (a) contacting a population of host cells with a population of viral vectors comprising the gene of interest (the starting heterologous gene modifying polypeptide or fragment or domain thereof), wherein: (1) the host cell is amenable to infection by the viral vector; (2) the host cell expresses viral genes required for the generation of viral particles; (3) the expression of at least one viral gene required for the production of an infectious viral particle is dependent on a function of the gene of interest; and/or (4) the viral vector allows for expression of the protein in the host cell, and can be replicated and packaged into a viral particle by the host cell. In some embodiments, the method comprises (b) contacting the host cells with a mutagen, using host cells with mutations that elevate mutation rate (e.g., either by carrying a mutation plasmid or some genome modification—e.g., proofing-impaired DNA polymerase, SOS genes, such as UmuC, UmuD′, and/or RecA, which mutations, if plasmid-bound, may be under control of an inducible promoter), or a combination thereof. In some embodiments, the method comprises (c) incubating the population of host cells under conditions allowing for viral replication and the production of viral particles, wherein host cells are removed from the host cell population, and fresh, uninfected host cells are introduced into the population of host cells, thus replenishing the population of host cells and creating a flow of host cells. In some embodiments, the cells are incubated under conditions allowing for the gene of interest to acquire a mutation. In some embodiments, the method further comprises (d) isolating a mutated version of the viral vector, encoding an evolved gene product (e.g., an evolved variant heterologous gene modifying polypeptide, or fragment or domain thereof), from the population of host cells.

The skilled artisan will appreciate a variety of features employable within the above-described framework. For example, in some embodiments, the viral vector or the phage is a filamentous phage, for example, an M13 phage, e.g., an M13 selection phage. In certain embodiments, the gene required for the production of infectious viral particles is the M13 gene III (gIII). In embodiments, the phage may lack a functional gIII, but otherwise comprise gI, gII, gIV, gV, gVI, gVII, gVIII, gIX, and a gX. In some embodiments, the generation of infectious VSV particles involves the envelope protein VSV-G. Various embodiments can use different retroviral vectors, for example, Murine Leukemia Virus vectors, or Lentiviral vectors. In embodiments, the retroviral vectors can efficiently be packaged with VSV-G envelope protein, e.g., as a substitute for the native envelope protein of the virus.

In some embodiments, host cells are incubated according to a suitable number of viral life cycles, e.g., at least 10, at least 20, at least 30, at least 40, at least 50, at least 100, at least 200, at least 300, at least 400, at least, 500, at least 600, at least 700, at least 800, at least 900, at least 1000, at least 1250, at least 1500, at least 1750, at least 2000, at least 2500, at least 3000, at least 4000, at least 5000, at least 7500, at least 10000, or more consecutive viral life cycles, which in on illustrative and non-limiting examples of M13 phage is 10-20 minutes per virus life cycle. Similarly, conditions can be modulated to adjust the time a host cell remains in a population of host cells, e.g., about 10, about 11, about 12, about 13, about 14, about 15, about 16, about 17, about 18, about 19, about 20, about 21, about 22, about 23, about 24, about 25, about 30, about 35, about 40, about 45, about 50, about 55, about 60, about 70, about 80, about 90, about 100, about 120, about 150, or about 180 minutes. Host cell populations can be controlled in part by density of the host cells, or, in some embodiments, the host cell density in an inflow, e.g., 10³ cells/ml, about 10⁴ cells/ml, about 10⁵ cells/ml, about 5-10⁵ cells/ml, about 10⁶ cells/ml, about 5-10⁶ cells/ml, about 10⁷ cells/ml, about 5-10⁷ cells/ml, about 10⁸ cells/ml, about 5-10⁸ cells/ml, about 10⁹ cells/ml, about 5·10⁹ cells/ml, about 10¹⁰ cells/ml, or about 5·10¹⁰ cells/ml.

### Inteins

In some embodiments, as described in more detail below, an intein-N (intN) domain may be fused to the N-terminal portion of a first domain of a heterologous gene modifying polypeptide described herein, and an intein-C (intC) domain may be fused to the C-terminal portion of a second domain of a heterologous gene modifying polypeptide described herein for the joining of the N-terminal portion to the C-terminal portion, thereby joining the first and second domains. In some embodiments, the first and second domains are each independently chosen from a DNA binding domain, an RNA binding domain, an RT domain, and an endonuclease domain.

Inteins can occur as self-splicing protein intron (e.g., peptide), e.g., which ligates flanking N-terminal and C-terminal exteins (e.g., fragments to be joined). An intein may, in some instances, comprise a fragment of a protein that is able to excise itself and join the remaining fragments (the exteins) with a peptide bond in a process known as protein splicing. Inteins are also referred to as “protein introns.” The process of an intein excising itself and joining the remaining portions of the protein is herein termed “protein splicing” or “intein-mediated protein splicing.”

In some embodiments, an intein of a precursor protein (an intein containing protein prior to intein-mediated protein splicing) comes from two genes. Such intein is referred to herein as a split intein (e.g., split intein-N and split intein-C). Accordingly, an intein-based approach may be used to join a first polypeptide sequence and a second polypeptide sequence together. For example, in cyanobacteria, DnaE, the catalytic subunit a of DNA polymerase III, is encoded by two separate genes, dnaE-n and dnaE-c. An intein-N domain, such as that encoded by the dnaE-n gene, when situated as part of a first polypeptide sequence, may join the first polypeptide sequence with a second polypeptide sequence, wherein the second polypeptide sequence comprises an intein-C domain, such as that encoded by the dnaE-c gene. Accordingly, in some embodiments, a protein can be made by providing nucleic acid encoding the first and second polypeptide sequences (e.g., wherein a first nucleic acid molecule encodes the first polypeptide sequence and a second nucleic acid molecule encodes the second polypeptide sequence), and the nucleic acid is introduced into the cell under conditions that allow for production of the first and second polypeptide sequences, and for joining of the first to the second polypeptide sequence via an intein-based mechanism.

Use of inteins for joining heterologous protein fragments is described, for example, in Wood et al., J. Biol. Chem. 289 (21); 14512-9 (2014) (incorporated herein by reference in its entirety). For example, when fused to separate protein fragments, the inteins IntN and IntC may recognize each other, splice themselves out, and/or simultaneously ligate the flanking N- and C-terminal exteins of the protein fragments to which they were fused, thereby reconstituting a full-length protein from the two protein fragments.

In some embodiments, a synthetic intein based on the dnaE intein, the Cfa-N (e.g., split intein-N) and Cfa-C (e.g., split intein-C) intein pair, is used. Examples of such inteins have been described, e.g., in Stevens et al., J Am Chem Soc. 2016 Feb. 24; 138 (7): 2162-5 (incorporated herein by reference in its entirety). Non-limiting examples of intein pairs that may be used in accordance with the present disclosure include: Cfa DnaE intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference.

In some embodiments involving a split Cas9, an intein-N domain and an intein-C domain may be fused to the N-terminal portion of the split Cas9 and the C-terminal portion of a split Cas9, respectively, for the joining of the N-terminal portion of the split Cas9 and the C-terminal portion of the split Cas9. For example, in some embodiments, an intein-N is fused to the C-terminus of the N-terminal portion of the split Cas9, i.e., to form a structure of N—[N-terminal portion of the split Cas9]-[intein-N]~ C. In some embodiments, an intein-C is fused to the N-terminus of the C-terminal portion of the split Cas9, i.e., to form a structure of N-[intein-C]~ [C-terminal portion of the split Cas9]-C. The mechanism of intein-mediated protein splicing for joining the proteins the inteins are fused to (e.g., split Cas9) is described in Shah et al., Chem Sci. 2014; 5 (1): 446-461, incorporated herein by reference. Methods for designing and using inteins are known in the art and described, for example by WO2020051561, WO2014004336, WO2017132580, US20150344549, and US20180127780, each of which is incorporated herein by reference in their entirety.

In some embodiments, a split refers to a division into two or more fragments. In some embodiments, a split Cas9 protein or split Cas9 comprises a Cas9 protein that is provided as an N-terminal fragment and a C-terminal fragment encoded by two separate nucleotide sequences. The polypeptides corresponding to the N-terminal portion and the C-terminal portion of the Cas9 protein may be spliced to form a reconstituted Cas9 protein. In embodiments, the Cas9 protein is divided into two fragments within a disordered region of the protein, e.g., as described in Nishimasu et al., Cell, Volume 156, Issue 5, pp. 935-949, 2014, or as described in Jiang et al. (2016) Science 351:867-871 and PDB file: 5F9R (each of which is incorporated herein by reference in its entirety). A disordered region may be determined by one or more protein structure determination techniques known in the art, including, without limitation, X-ray crystallography, NMR spectroscopy, electron microscopy (e.g., cryoEM), and/or in silico protein modeling. In some embodiments, the protein is divided into two fragments at any C, T, A, or S, e.g., within a region of SpCas9 between amino acids A292-G364, F445-K483, or E565-T637, or at corresponding positions in any other Cas9, Cas9 variant (e.g., nCas9, dCas9), or other napDNAbp. In some embodiments, protein is divided into two fragments at SpCas9 T310, T313, A456, S469, or C574. In some embodiments, the process of dividing the protein into two fragments is referred to as splitting the protein.

In some embodiments, a protein fragment ranges from about 2-1000 amino acids (e.g., between 2-10, 10-50, 50-100, 100-200, 200-300, 300-400, 400-500, 500-600, 600-700, 700-800, 800-900, or 900-1000 amino acids) in length. In some embodiments, a protein fragment ranges from about 5-500 amino acids (e.g., between 5-10, 10-50, 50-100, 100-200, 200-300, 300-400, or 400-500 amino acids) in length. In some embodiments, a protein fragment ranges from about 20-200 amino acids (e.g., between 20-30, 30-40, 40-50, 50-100, or 100-200 amino acids) in length.

In some embodiments, a portion or fragment of a heterologous gene modifying polypeptide is fused to an intein. The nuclease can be fused to the N-terminus or the C-terminus of the intein. In some embodiments, a portion or fragment of a fusion protein is fused to an intein and fused to an AAV capsid protein. The intein, nuclease, and capsid protein can be fused together in any arrangement (e.g., nuclease-intein-capsid, intein-nuclease-capsid, capsid-intein-nuclease, etc.). In some embodiments, the N-terminus of an intein is fused to the C-terminus of a fusion protein and the C-terminus of the intein is fused to the N-terminus of an AAV capsid protein.

In some embodiments, an endonuclease domain (e.g., a nickase Cas9 domain) is fused to intein-N and a polypeptide comprising an RT domain is fused to an intein-C.

Exemplary nucleotide and amino acid sequences of intein-N domains and compatible intein-C domains are provided below:

DnaE Intein-N DNA:
(SEQ ID NO: 5029)
TGCCTGTCATACGAAACCGAGATACTGACAGTAGAATATGGCCTTCTGCCAATCGGG
AAGATTGTGGAGAAACGGATAGAATGCACAGTTTACTCTGTCGATAACAATGGTAA
CATTTATACTCAGCCAGTTGCCCAGTGGCACGACCGGGGAGAGCAGGAAGTATTCG
AATACTGTCTGGAGGATGGAAGTCTCATTAGGGCCACTAAGGACCACAAATTTATG
ACAGTCGATGGCCAGATGCTGCCTATAGACGAAATCTTTGAGCGAGAGTTGGACCTC
ATGCGAGTTGACAACCTTCCTAAT
DnaE Intein-N Protein:
(SEQ ID NO: 5030)
CLSYETEILTVEYGLLPIGKIVEKRIECTVYSVDNNGNIYTQPVAQWHDRGEQEVFEYCL
EDGSLIRATKDHKFMTVDGQMLPIDEIFERELDLMRVDNLPN
DnaE Intein-C DNA:
(SEQ ID NO: 5031)
ATGATCAAGATAGCTACAAGGAAGTATCTTGGCAAACAAAACGTTTATGATATTGG
AGTCGAAAGAGATCACAACTTTGCTCTGAAGAACGGATTCATAGCTTCTAAT
DnaE Intein-C Protein:
(SEQ ID NO: 5032)
MIKIATRKYLGKQNVYDIGVERDHNFALKNGFIASN
Cfa-N DNA:
(SEQ ID NO: 5033)
TGCCTGTCTTATGATACCGAGATACTTACCGTTGAATATGGCTTCTTGCCTATTGGAA
AGATTGTCGAAGAGAGAATTGAATGCACAGTATATACTGTAGACAAGAATGGTTTC
GTTTACACACAGCCCATTGCTCAATGGCACAATCGCGGCGAACAAGAAGTATTTGA
GTACTGTCTCGAGGATGGAAGCATCATACGAGCAACTAAAGATCATAAATTCATGA
CCACTGACGGGCAGATGTTGCCAATAGATGAGATATTCGAGCGGGGCTTGGATCTC
AAACAAGTGGATGGATTG CCA
Cfa-N Protein:
(SEQ ID NO: 5034)
CLSYDTEILTVEYGFLPIGKIVEERIECTVYTVDKNGFVYTQPIAQWHNRGEQEVFEYCL
EDGSIIRATKDHKFMTTDGQMLPIDEIFERGLDLKQVDGLP
Cfa-C DNA:
(SEQ ID NO: 5035)
ATGAAGAGGACTGCCGATGGATCAGAGTTTGAATCTCCCAAGAAGAAGAGGAAAGT
AAAGATAATATCTCGAAAAAGTCTTGGTACCCAAAATGTCTATGATATTGGAGTGGA
GAAAGATCACAACTTCCTTCTCAAGAACGGTCTCGTAGCCAGCAAC
Cfa-C Protein:
(SEQ ID NO: 5036)
MKRTADGSEFESPKKKRKVKIISRKSLGTQNVYDIGVEKDHNFLLKNGLVASN

### Additional Domains

The heterologous gene modifying polypeptide can bind a target DNA sequence and template nucleic acid (e.g., template RNA), nick the target site, and write (e.g., reverse transcribe) the template into DNA, resulting in a modification of the target site. In some embodiments, additional domains may be added to the polypeptide to enhance the efficiency of the process. In some embodiments, the heterologous gene modifying polypeptide may contain an additional DNA ligation domain to join reverse transcribed DNA to the DNA of the target site. In some embodiments, the polypeptide may comprise a heterologous RNA-binding domain. In some embodiments, the polypeptide may comprise a domain having 5′ to 3′ exonuclease activity (e.g., wherein the 5′ to 3′ exonuclease activity increases repair of the alteration of the target site, e.g., in favor of alteration over the original genomic sequence). In some embodiments, the polypeptide may comprise a domain having 3′ to 5′ exonuclease activity, e.g., proof-reading activity. In some embodiments, the writing domain, e.g., RT domain, has 3′ to 5′ exonuclease activity, e.g., proof-reading activity.

### Template Nucleic Acids

The heterologous gene modifying systems described herein can modify a host target DNA site using a template nucleic acid sequence. In some embodiments, the heterologous gene modifying systems described herein transcribe an RNA sequence template into host target DNA sites by target-primed reverse transcription (TPRT). By modifying DNA sequence(s) via reverse transcription of the RNA sequence template directly into the host genome, the heterologous gene modifying system can insert an object sequence into a target genome without the need for exogenous DNA sequences to be introduced into the host cell (unlike, for example, CRISPR systems), as well as eliminate an exogenous DNA insertion step. The heterologous gene modifying system can also delete a sequence from the target genome or introduce a substitution using an object sequence. Therefore, the heterologous gene modifying system provides a platform for the use of customized RNA sequence templates containing object sequences, e.g., sequences comprising heterologous gene coding and/or function information.

In some embodiments, the template nucleic acid comprises one or more sequence (e.g., 2 sequences) that binds the heterologous gene modifying polypeptide.

In some embodiments a system or method described herein comprises a single template nucleic acid (e.g., template RNA). In some embodiments a system or method described herein comprises a plurality of template nucleic acids (e.g., template RNAs). For example, a system described herein comprises a first RNA comprising (e.g., from 5′ to 3′) a sequence that binds the heterologous gene modifying polypeptide (e.g., the DNA-binding domain and/or the endonuclease domain, e.g., a gRNA) and a sequence that binds a target site (e.g., a second strand of a site in a target genome), and a second RNA (e.g., a template RNA) comprising (e.g., from 5′ to 3′) optionally a sequence that binds the heterologous gene modifying polypeptide (e.g., that specifically binds the RT domain), a heterologous object sequence, and a PBS sequence. In some embodiments, when the system comprises a plurality of nucleic acids, each nucleic acid comprises a conjugating domain. In some embodiments, a conjugating domain enables association of nucleic acid molecules, e.g., by hybridization of complementary sequences. For example, in some embodiments a first RNA comprises a first conjugating domain and a second RNA comprises a second conjugating domain, and the first and second conjugating domains are capable of hybridizing to one another, e.g., under stringent conditions. In some embodiments, the stringent conditions for hybridization include hybridization in 4× sodium chloride/sodium citrate (SSC), at about 65 C, followed by a wash in 1×SSC, at about 65 C.

In some embodiments, the template nucleic acid comprises RNA. In some embodiments, the template nucleic acid comprises DNA (e.g., single stranded or double stranded DNA).

In some embodiments, the template nucleic acid comprises one or more (e.g., 2) homology domains that have homology to the target sequence. In some embodiments, the homology domains are about 10-20, 20-50, or 50-100 nucleotides in length.

In some embodiments, a template RNA can comprise a gRNA sequence, e.g., to direct the heterologous gene modifying polypeptide to a target site of interest. In some embodiments, a template RNA comprises (e.g., from 5′ to 3′) (i) optionally a gRNA spacer that binds a target site (e.g., a second strand of a site in a target genome), (ii) optionally a gRNA scaffold that binds a polypeptide described herein (e.g., a heterologous gene modifying polypeptide or a Cas polypeptide), (iii) a heterologous object sequence comprising a mutation region (optionally the heterologous object sequence comprises, from 5′ to 3′, a first homology region, a mutation region, and a second homology region), and (iv) a primer binding site (PBS) sequence comprising a 3′ target homology domain.

The template nucleic acid (e.g., template RNA) component of a genome editing system described herein typically is able to bind the heterologous gene modifying polypeptide of the system. In some embodiments the template nucleic acid (e.g., template RNA) has a 3′ region that is capable of binding a heterologous gene modifying polypeptide. The binding region, e.g., 3′ region, may be a structured RNA region, e.g., having at least 1, 2, or 3 hairpin loops, capable of binding the heterologous gene modifying polypeptide of the system. The binding region may associate the template nucleic acid (e.g., template RNA) with any of the polypeptide modules. In some embodiments, the binding region of the template nucleic acid (e.g., template RNA) may associate with an RNA-binding domain in the polypeptide. In some embodiments, the binding region of the template nucleic acid (e.g., template RNA) may associate with the reverse transcription domain of the heterologous gene modifying polypeptide (e.g., specifically bind to the RT domain). In some embodiments, the template nucleic acid (e.g., template RNA) may associate with the DNA binding domain of the polypeptide, e.g., a gRNA associating with a Cas9-derived DNA binding domain. In some embodiments, the binding region may also provide DNA target recognition, e.g., a gRNA hybridizing to the target DNA sequence and binding the polypeptide, e.g., a Cas9 domain. In some embodiments, the template nucleic acid (e.g., template RNA) may associate with multiple components of the polypeptide, e.g., DNA binding domain and reverse transcription domain.

In some embodiments the template RNA has a poly-A tail at the 3′ end. In some embodiments the template RNA does not have a poly-A tail at the 3′ end.

In some embodiments, the template nucleic acid is a template RNA. In some embodiments, the template RNA comprises one or more modified nucleotides. For example, in some embodiments, the template RNA comprises one or more deoxyribonucleotides. In some embodiments, regions of the template RNA are replaced by DNA nucleotides, e.g., to enhance stability of the molecule. For example, the 3′ end of the template may comprise DNA nucleotides, while the rest of the template comprises RNA nucleotides that can be reverse transcribed. For instance, in some embodiments, the heterologous object sequence is primarily or wholly made up of RNA nucleotides (e.g., at least 90%, 95%, 98%, or 99% RNA nucleotides). In some embodiments, the PBS sequence is primarily or wholly made up of DNA nucleotides (e.g., at least 90%, 95%, 98%, or 99% DNA nucleotides). In other embodiments, the heterologous object sequence for writing into the genome may comprise DNA nucleotides. In some embodiments, the DNA nucleotides in the template are copied into the genome by a domain capable of DNA-dependent DNA polymerase activity. In some embodiments, the DNA-dependent DNA polymerase activity is provided by a DNA polymerase domain in the polypeptide. In some embodiments, the DNA-dependent DNA polymerase activity is provided by a reverse transcriptase domain that is also capable of DNA-dependent DNA polymerization, e.g., second strand synthesis. In some embodiments, the template molecule is composed of only DNA nucleotides.

In some embodiments, a system described herein comprises two nucleic acids which together comprise the sequences of a template RNA described herein. In some embodiments, the two nucleic acids are associated with each other non-covalently, e.g., directly associated with each other (e.g., via base pairing), or indirectly associated as part of a complex comprising one or more additional molecule.

A template RNA described herein may comprise, from 5′ to 3′: (1) a gRNA spacer; (2) a gRNA scaffold; (3) heterologous object sequence (4) a primer binding site (PBS) sequence. Each of these components is now described in more detail.

gRNA Spacer and gRNA Scaffold

A template RNA described herein may comprise a gRNA spacer that directs the heterologous gene modifying system to a target nucleic acid, and a gRNA scaffold that promotes association of the template RNA with the Cas domain of the heterologous gene modifying polypeptide. The systems described herein can also comprise a gRNA that is not part of a template nucleic acid. For example, a gRNA that comprises a gRNA spacer and gRNA scaffold, but not a heterologous object sequence or a PBS sequence, can be used, e.g., to induce second strand nicking, e.g., as described in the section herein entitled “Second Strand Nicking”.

In some embodiments, the gRNA is a short synthetic RNA composed of a scaffold sequence that participates in CRISPR-associated protein binding and a user-defined ~20 nucleotide targeting sequence for a genomic target. The structure of a complete gRNA was described by Nishimasu et al. Cell 156, P935-949 (2014). The gRNA (also referred to as sgRNA for single-guide RNA) consists of crRNA- and tracrRNA-derived sequences connected by an artificial tetraloop. The crRNA sequence can be divided into guide (20 nt) and repeat (12 nt) regions, whereas the tracrRNA sequence can be divided into anti-repeat (14 nt) and three tracrRNA stem loops (Nishimasu et al. Cell 156, P935-949 (2014)). In practice, guide RNA sequences are generally designed to have a length of between 17-24 nucleotides (e.g., 19, 20, or 21 nucleotides) and be complementary to a targeted nucleic acid sequence. Custom gRNA generators and algorithms are available commercially for use in the design of effective guide RNAs. In some embodiments, the gRNA comprises two RNA components from the native CRISPR system, e.g. crRNA and tracrRNA. As is well known in the art, the gRNA may also comprise a chimeric, single guide RNA (sgRNA) containing sequence from both a tracrRNA (for binding the nuclease) and at least one crRNA (to guide the nuclease to the sequence targeted for editing/binding). Chemically modified sgRNAs have also been demonstrated to be effective for use with CRISPR-associated proteins; see, for example, Hendel et al. (2015) Nature Biotechnol., 985-991. In some embodiments, a gRNA spacer comprises a nucleic acid sequence that is complementary to a DNA sequence associated with a target gene.

In some embodiments, the region of the template nucleic acid, e.g., template RNA, comprising the gRNA adopts an underwound ribbon-like structure of gRNA bound to target DNA (e.g., as described in Mulepati et al. Science 19 Sep. 2014: Vol. 345, Issue 6203, pp. 1479-1484). Without wishing to be bound by theory, this non-canonical structure is thought to be facilitated by rotation of every sixth nucleotide out of the RNA-DNA hybrid. Thus, in some embodiments, the region of the template nucleic acid, e.g., template RNA, comprising the gRNA may tolerate increased mismatching with the target site at some interval, e.g., every sixth base. In some embodiments, the region of the template nucleic acid, e.g., template RNA, comprising the gRNA comprising homology to the target site may possess wobble positions at a regular interval, e.g., every sixth base, that do not need to base pair with the target site.

In some embodiments, the template nucleic acid (e.g., template RNA) has at least 15, 16, 17, 18, 19, 20, 21, 22, 23, or 24 bases of at least 80%, 85%, 90%, 95%, 99%, or 100% homology to the target site, e.g., at the 5′ end, e.g., comprising a gRNA spacer sequence of length appropriate to the Cas9 domain of the heterologous gene modifying polypeptide (Table 8).

In some embodiments, a Cas9 derivative with enhanced activity may be used in the gene modification polypeptide. In some embodiments, a Cas9 derivative may comprise mutations that improve activity of the HNH endonuclease domain, e.g., SpyCas9 R221K, N394K, or mutations that improve R-loop formation, e.g., SpyCas9 L1245V, or comprise a combination of such mutations, e.g., SpyCas9 R221K/N394K, SpyCas9 N394K/L1245V, SpyCas9 R221K/L1245V, or SpyCas9 R221K/N394K/L1245V (see, e.g., Spencer and Zhang Sci Rep 7:16836 (2017), the Cas9 derivatives and comprising mutations of which are incorporated herein by reference). In some embodiments, a Cas9 derivative may comprise one or more types of mutations described herein, e.g., PAM-modifying mutations, protein stabilizing mutations, activity enhancing mutations, and/or mutations partially or fully inactivating one or two endonuclease domains relative to the parental enzyme (e.g., one or more mutations to abolish endonuclease activity towards one or both strands of a target DNA, e.g., a nickase or catalytically dead enzyme). In some embodiments, a Cas9 enzyme used in a system described herein may comprise mutations that confer nickase activity toward the enzyme (e.g., SpyCas9 N863A or H840A) in addition to mutations improving catalytic efficiency (e.g., SpyCas9 R221K, N394K, and/or L1245V). In some embodiments, a Cas9 enzyme used in a system described herein is a SpyCas9 enzyme or derivative that further comprises an N863A mutation to confer nickase activity in addition to R221K and N394K mutations to improve catalytic efficiency.

Table 12 provides parameters to define components for designing gRNA and/or Template RNAs to apply Cas variants listed in Table 8 for gene modifying. The cut site indicates the validated or predicted protospacer adjacent motif (PAM) requirements, validated or predicted location of cut site (relative to the most upstream base of the PAM site). The gRNA for a given enzyme can be assembled by concatenating the crRNA, Tetraloop, and tracrRNA sequences, and further adding a 5′ spacer of a length within Spacer (min) and Spacer (max) that matches a protospacer at a target site. Further, the predicted location of the ssDNA nick at the target is important for designing a PBS sequence of a Template RNA that can anneal to the sequence immediately 5′ of the nick in order to initiate target primed reverse transcription. In some embodiments, a gRNA scaffold described herein comprises a nucleic acid sequence comprising, in the 5′ to 3′ direction, a crRNA of Table 12, a tetraloop from the same row of Table 12, and a tracrRNA from the same row of Table 12, or a sequence having at least 70%, 80%, 85%, 90%, 95%, or 99% identity thereto. In some embodiments, the gRNA or template RNA comprising the scaffold further comprises a gRNA spacer having a length within the Spacer (min) and Spacer (max) indicated in the same row of Table 12. In some embodiments, the gRNA or template RNA having a sequence according to Table 12 is comprised by a system that further comprises a heterologous gene modifying polypeptide, wherein the heterologous gene modifying polypeptide comprises a Cas domain described in the same row of Table 12.

TABLE 12
Parameters to define components for designing gRNA and/or Template RNAs to apply Cas variants
listed in Table 8 in heterologous gene modifying systems

| | | | | Spacer | Spacer | | SEQ ID | Tetra- | | SEQ ID |
|---|---|---|---|---|---|---|---|---|---|---|
| Variant | PAM(s) | Cut | Tier | (min) | (max) | crRNA | NO: | loop | tracrRNA | NO: |
| Nme2Cas9 | NNNNCC | -3 | 1 | 22 | 24 | GTTGTAGC | 10,051 | GAAA | CGAAATGAGAACCGTTGC | 10,151 |
| | | | | | | TCCCTTTCT | | | TACAATAAGGCCGTCTGA | |
| | | | | | | CATTTCG | | | AAAGATGTGCCGCAACGC | |
| | | | | | | | | | TCTGCCCCTTAAAGCTTC | |
| | | | | | | | | | TGCTTTAAGGGGCATCGT | |
| | | | | | | | | | TTA | |
| PpnCas9 | NNNNRTT | | 1 | 21 | 24 | GTTGTAGC | 10,052 | GAAA | GCGAAATGAAAAACGTTG | 10,152 |
| | | | | | | TCCCTTTTT | | | TTACAATAAGAGATGAAT | |
| | | | | | | CATTTCGC | | | TTCTCGCAAAGCTCTGCC | |
| | | | | | | | | | TCTTGAAATTTCGGTTTC | |
| | | | | | | | | | AAGAGGCATC | |
| SauCas9 | NNGRR; | -3 | 1 | 21 | 23 | GTTTTAGT | 10,053 | GAAA | CAGAATCTACTAAAACAA | 10,153 |
| | NNGRRT | | | | | ACTCTG | | | GGCAAAATGCCGTGTTTA | |
| | | | | | | | | | TCTCGTCAACTTGTTGGC | |
| | | | | | | | | | GAGA | |
| SauCas9- | NNNRR; | -3 | 1 | 21 | 21 | GTTTTAGT | 10,054 | GAAA | CAGAATCTACTAAAACAA | 10,154 |
| KKH | NNNRRT | | | | | ACTCTG | | | GGCAAAATGCCGTGTTTA | |
| | | | | | | | | | TCTCGTCAACTTGTTGGC | |
| | | | | | | | | | GAGA | |
| SauriCas9 | NNGG | -3 | 1 | 21 | 21 | GTTTTAGT | 10,055 | GAAA | CAGAATCTACTAAAACAA | 10,155 |
| | | | | | | ACTCTG | | | GGCAAAATGCCGTGTTTA | |
| | | | | | | | | | TCTCGTCAACTTGTTGGC | |
| | | | | | | | | | GAGA | |
| SauriCas9- | NNRG | -3 | 1 | 21 | 21 | GTTTTAGT | 10,056 | GAAA | CAGAATCTACTAAAACAA | 10,156 |
| KKH | | | | | | ACTCTG | | | GGCAAAATGCCGTGTTTA | |
| | | | | | | | | | TCTCGTCAACTTGTTGGC | |
| | | | | | | | | | GAGA | |
| ScaCas9- | NNG | -3 | 1 | 20 | 20 | GTTTTAGA | 10,057 | GAAA | TAGCAAGTTAAAATAAGG | 10,157 |
| Sc++ | | | | | | GCTA | | | CTAGTCCGTTATCAACTT | |
| | | | | | | | | | GAAAAAGTGGCACCGAGT | |
| | | | | | | | | | CGGTGC | |
| SpyCas9 | NGG | -3 | 1 | 20 | 20 | GTTTTAGA | 10,058 | GAAA | TAGCAAGTTAAAATAAGG | 10,158 |
| | | | | | | GCTA | | | CTAGTCCGTTATCAACTT | |
| | | | | | | | | | GAAAAAGTGGCACCGAGT | |
| | | | | | | | | | CGGTGC | |
| SpyCas9_ | NGG | -3 | 1 | 20 | 20 | GTTTTAGA | 10,058 | GAAA | TAGCAAGTTAAAATAAGG | 10,193 |
| i_v1 | | | | | | GCTA | | | CTAGTCCGTTATCAACTT | |
| | | | | | | | | | GGACTTCGGTCCAAGTGG | |
| | | | | | | | | | CACCGAGTCGGTGC | |
| SpyCas9_ | NGG | -3 | 1 | 20 | 20 | GTTTTAGA | 10,058 | GAAA | TAGCAAGTTAAAATAAGG | 10,194 |
| i_v2 | | | | | | GCTA | | | CTAGTCCGTTATCAACTT | |
| | | | | | | | | | GGAGCTTGCTCCAAGTGG | |
| | | | | | | | | | CACCGAGTCGGTGC | |
| SpyCas9_ | NGG | -3 | 1 | 20 | 20 | GTTTTAGA | 10,058 | GAAA | GTTTTAGAGCTAGAAATA | 10,195 |
| i_v3 | | | | | | GCTA | | | GCAAGTTAAAATAAGGCT | |
| | | | | | | | | | AGTCCGTTATCGACTTGA | |
| | | | | | | | | | AAAAGTCGCACCGAGTCG | |
| | | | | | | | | | GTGC | |
| SpyCas9-NG | NG | -3 | 1 | 20 | 20 | GTTTTAGA | 10,059 | GAAA | TAGCAAGTTAAAATAAGG | 10,159 |
| | (NGG = NG | | | | | GCTA | | | CTAGTCCGTTATCAACTT | |
| | A = NGT > | | | | | | | | GAAAAAGTGGCACCGAGT | |
| | NGC) | | | | | | | | CGGTGC | |
| SpyCas9- | NRN > NYN | -3 | 1 | 20 | 20 | GTTTTAGA | 10,060 | GAAA | TAGCAAGTTAAAATAAGG | 10,160 |
| SpRY | | | | | | GCTA | | | CTAGTCCGTTATCAACTT | |
| | | | | | | | | | GAAAAAGTGGCACCGAGT | |
| | | | | | | | | | CGGTGC | |
| St1Cas9 | NNAGAA | -3 | 1 | 20 | 20 | GTCTTTGTA | 10,061 | GTAC | CAGAAGCTACAAAGATAA | 10,161 |
| | W > NNAG | | | | | CTCTG | | | GGCTTCATGCCGAAATCA | |
| | GAW = NN | | | | | | | | ACACCCTGTCATTTTATG | |
| | GGAAW | | | | | | | | GCAGGGTGTTTT | |
| BlatCas9 | NNNNCN | -3 | 1 | 19 | 23 | GCTATAGT | 10,062 | GAAA | GGTAAGTTGCTATAGTAA | 10,162 |
| | AA > NNNN | | | | | TCCTTACT | | | GGGCAACAGACCCGAGGC | |
| | CNDD > NN | | | | | | | | GTTGGGGATCGCCTAGCC | |
| | NNC | | | | | | | | CGTGTTTACGGGCTCTCC | |
| | | | | | | | | | CCATATTCAAAATAATGA | |
| | | | | | | | | | CAGACGAGCACCTTGGAG | |
| | | | | | | | | | CATTTATCTCCGAGGTGC | |
| | | | | | | | | | T | |
| cCas9-v16 | NNVACT; | -3 | 2 | 21 | 21 | GTCTTAGT | 10,063 | GAAA | CAGAATCTACTAAGACAA | 10,163 |
| | NNVATGM; | | | | | ACTCTG | | | GGCAAAATGCCGTGTTTA | |
| | NNVATT; | | | | | | | | TCTCGTCAACTTGTTGGC | |
| | NNVGCT; | | | | | | | | GAGA | |
| | NNVGTG; | | | | | | | | | |
| | NNVGTT | | | | | | | | | |
| cCas9-v17 | NNVRRN | -3 | 2 | 21 | 21 | GTCTTAGT | 10,064 | GAAA | CAGAATCTACTAAGACAA | 10,164 |
| | | | | | | ACTCTG | | | GGCAAAATGCCGTGTTTA | |
| | | | | | | | | | TCTCGTCAACTTGTTGGC | |
| | | | | | | | | | GAGA | |
| cCas9-v21 | NNVACT; | -3 | 2 | 21 | 21 | GTCTTAGT | 10,065 | GAAA | CAGAATCTACTAAGACAA | 10,165 |
| | NNVATGM; | | | | | ACTCTG | | | GGCAAAATGCCGTGTTTA | |
| | NNVATT; | | | | | | | | TCTCGTCAACTTGTTGGC | |
| | NNVGCT | | | | | | | | GAGA | |
| | NNVGTG; | | | | | | | | | |
| | NNVGTT | | | | | | | | | |
| cCas9-v42 | NNVRRN | -3 | 2 | 21 | 21 | GTCTTAGT | 10,066 | GAAA | CAGAATCTACTAAGACAA | 10,166 |
| | | | | | | ACTCTG | | | GGCAAAATGCCGTGTTTA | |
| | | | | | | | | | TCTCGTCAACTTGTTGGC | |
| | | | | | | | | | GAGA | |
| CdiCas9 | NNRHHHY; | | 2 | 22 | 22 | ACTGGGGT | 10,067 | GAAA | CTGAACCTCAGTAAGCAT | 10,167 |
| | NNRAAA | | | | | TCAG | | | TGGCTCGTTTCCAATGTT | |
| | Y | | | | | | | | GATTGCTCCGCCGGTGCT | |
| | | | | | | | | | CCTTATTTTTAAGGGCGC | |
| | | | | | | | | | CGGC | |
| CjeCas9 | NNNNRYA | -3 | 2 | 21 | 23 | GTTTTAGTC | 10,068 | GAAA | AGGGACTAAAATAAAGAG | 10,168 |
| | C | | | | | CCT | | | TTTGCGGGACTCTGCGGG | |
| | | | | | | | | | GTTACAATCCCCTAAAAC | |
| | | | | | | | | | CGC | |
| GeoCas9 | NNNNCRA | | 2 | 21 | 23 | GTCATAGT | 10,069 | GAAA | TCAGGGTTACTATGATAA | 10,169 |
| | A | | | | | TCCCCTGA | | | GGGCTTTCTGCCTAAGGC | |
| | | | | | | | | | AGACTGACCCGCGGCGTT | |
| | | | | | | | | | GGGGATCGCCTGTCGCCC | |
| | | | | | | | | | GCTTTTGGGGGCATTCCC | |
| | | | | | | | | | CATCCTT | |
| iSpy | NAAN | -3 | 2 | 19 | 21 | GTTTTAGA | 10,070 | GAAA | TAGCAAGTTAAAATAAGG | 10,170 |
| MacCas9 | | | | | | GCTA | | | CTAGTCCGTTATCAACTT | |
| | | | | | | | | | GAAAAAGTGGCACCGAGT | |
| | | | | | | | | | CGGTGC | |
| NmeCas9 | NNNNGAYT; | -3 | 2 | 20 | 24 | GTTGTAGC | 10,071 | GAAA | CGAAATGAGAACCGTTGC | 10,171 |
| | NNNNGYTT; | | | | | TCCCTTTCT | | | TACAATAAGGCCGTCTGA | |
| | NNNNGAYA; | | | | | CATTTCG | | | AAAGATGTGCCGCAACGC | |
| | NNNNGTCT | | | | | | | | TCTGCCCCTTAAAGCTTC | |
| | | | | | | | | | TGCTTTAAGGGGCATCGT | |
| | | | | | | | | | TTA | |
| ScaCas9 | NNG | -3 | 2 | 20 | 20 | GTTTTAGA | 10,072 | GAAA | TAGCAAGTTAAAATAAGG | 10,172 |
| | | | | | | GCTA | | | CTAGTCCGTTATCAACTT | |
| | | | | | | | | | GAAAAAGTGGCACCGAGT | |
| | | | | | | | | | CGGTGC | |
| ScaCas9- | NNG | -3 | 2 | 20 | 20 | GTTTTAGA | 10,073 | GAAA | TAGCAAGTTAAAATAAGG | 10,173 |
| HiFi-Sc++ | | | | | | GCTA | | | CTAGTCCGTTATCAACTT | |
| | | | | | | | | | GAAAAAGTGGCACCGAGT | |
| | | | | | | | | | CGGTGC | |
| SpyCas9- | NRRH | -3 | 2 | 20 | 20 | GTTTAAGA | 10,074 | GAAA | CAGCATAGCAAGTTTAAA | 10,174 |
| 3var-NRRH | | | | | | GCTATGCT | | | TAAGGCTAGTCCGTTATC | |
| | | | | | | G | | | AACTTGAAAAAGTGGCAC | |
| | | | | | | | | | CGAGTCGGTGC | |
| SpyCas9- | NRTH | -3 | 2 | 20 | 20 | GTTTAAGA | 10,075 | GAAA | CAGCATAGCAAGTTTAAA | 10,175 |
| 3var-NRTH | | | | | | GCTATGCT | | | TAAGGCTAGTCCGTTATC | |
| | | | | | | G | | | AACTTGAAAAAGTGGCAC | |
| | | | | | | | | | CGAGTCGGTGC | |
| SpyCas9- | NRCH | -3 | 2 | 20 | 20 | GTTTAAGA | 10,076 | GAAA | CAGCATAGCAAGTTTAAA | 10,176 |
| 3var-NRCH | | | | | | GCTATGCT | | | TAAGGCTAGTCCGTTATC | |
| | | | | | | G | | | AACTTGAAAAAGTGGCAC | |
| | | | | | | | | | CGAGTCGGTGC | |
| SpyCas9- | NGG | -3 | 2 | 20 | 20 | GTTTTAGA | 10,077 | GAAA | TAGCAAGTTAAAATAAGG | 10,177 |
| HF1 | | | | | | GCTA | | | CTAGTCCGTTATCAACTT | |
| | | | | | | | | | GAAAAAGTGGCACCGAGT | |
| | | | | | | | | | CGGTGC | |
| SpyCas9- | NAAG | -3 | 2 | 20 | 20 | GTTTTAGA | 10,078 | GAAA | TAGCAAGTTAAAATAAGG | 10,178 |
| QQR1 | | | | | | GCTA | | | CTAGTCCGTTATCAACTT | |
| | | | | | | | | | GAAAAAGTGGCACCGAGT | |
| | | | | | | | | | CGGTGC | |
| SpyCas9- | NGN | -3 | 2 | 20 | 20 | GTTTTAGA | 10,079 | GAAA | TAGCAAGTTAAAATAAGG | 10,179 |
| SpG | | | | | | GCTA | | | CTAGTCCGTTATCAACTT | |
| | | | | | | | | | GAAAAAGTGGCACCGAGT | |
| | | | | | | | | | CGGTGC | |
| SpyCas9- | NGAN | -3 | 2 | 20 | 20 | GTTTTAGA | 10,080 | GAAA | TAGCAAGTTAAAATAAGG | 10,180 |
| VQR | | | | | | GCTA | | | CTAGTCCGTTATCAACTT | |
| | | | | | | | | | GAAAAAGTGGCACCGAGT | |
| | | | | | | | | | CGGTGC | |
| SpyCas9- | NGCG | -3 | 2 | 20 | 20 | GTTTTAGA | 10,081 | GAAA | TAGCAAGTTAAAATAAGG | 10,181 |
| VRER | | | | | | GCTA | | | CTAGTCCGTTATCAACTT | |
| | | | | | | | | | GAAAAAGTGGCACCGAGT | |
| | | | | | | | | | CGGTGC | |
| SpyCas9- | NG;GAA; | -3 | 2 | 20 | 20 | GTTTAAGA | 10,082 | GAAA | CAGCATAGCAAGTTTAAA | 10,182 |
| xCas | GAT | | | | | GCTATGCT | | | TAAGGCTAGTCCGTTATC | |
| | | | | | | G | | | AACTTGAAAAAGTGGCAC | |
| | | | | | | | | | CGAGTCGGTGC | |
| SpyCas9- | NG | -3 | 2 | 20 | 20 | GTTTAAGA | 10,083 | GAAA | CAGCATAGCAAGTTTAAA | 10,183 |
| xCas-NG | | | | | | GCTATGCT | | | TAAGGCTAGTCCGTTATC | |
| | | | | | | G | | | AACTTGAAAAAGTGGCAC | |
| | | | | | | | | | CGAGTCGGTGC | |
| St1Cas9- | NNACAA | -3 | 2 | 20 | 20 | GTCTTTGTA | 10,084 | GTAC | CAGAAGCTACAAAGATAA | 10,184 |
| CNRZ1066 | | | | | | CTCTG | | | GGCTTCATGCCGAAATCA | |
| | | | | | | | | | ACACCCTGTCATTTTATG | |
| | | | | | | | | | GCAGGGTGTTTT | |
| St1Cas9- | NNGCAA | -3 | 2 | 20 | 20 | GTCTTTGTA | 10,085 | GTAC | CAGAAGCTACAAAGATAA | 10,185 |
| LMG1831 | | | | | | CTCTG | | | GGCTTCATGCCGAAATCA | |
| | | | | | | | | | ACACCCTGTCATTTTATG | |
| | | | | | | | | | GCAGGGTGTTTT | |
| St1Cas9- | NNAAAA | -3 | 2 | 20 | 20 | GTCTTTGTA | 10,086 | GTAC | CAGAAGCTACAAAGATAA | 10,186 |
| MTH17CL396 | | | | | | CTCTG | | | GGCTTCATGCCGAAATCA | |
| | | | | | | | | | ACACCCTGTCATTTTATG | |
| | | | | | | | | | GCAGGGTGTTTT | |
| St1Cas9- | NNGAAA | -3 | 2 | 20 | 20 | GTCTTTGTA | 10,087 | GTAC | CAGAAGCTACAAAGATAA | 10,187 |
| TH1477 | | | | | | CTCTG | | | GGCTTCATGCCGAAATCA | |
| | | | | | | | | | ACACCCTGTCATTTTATG | |
| | | | | | | | | | GCAGGGTGTTTT | |
| sRGN3.1 | NNGG | | 1 | 21 | 23 | GTTTTAGT | 10,088 | GAAA | CAGAATCTACTGAAACAA | 10,188 |
| | | | | | | ACTCTG | | | GACAATATGTCGTGTTTA | |
| | | | | | | | | | TCCCATCAATTTATTGGT | |
| | | | | | | | | | GGGATTTT | |
| sRGN3.3 | NNGG | | 1 | 21 | 23 | GTTTTAGT | 10,089 | GAAA | CAGAATCTACTGAAACAA | 10,189 |
| | | | | | | ACTCTG | | | GACAATATGTCGTGTTTA | |
| | | | | | | | | | TCCCATCAATTTATTGGT | |
| | | | | | | | | | GGGATTTT | |

Herein, when an RNA sequence (e.g., a template RNA sequence) is said to comprise a particular sequence (e.g., a sequence of Table 12 or a portion thereof) that comprises thymine (T), it is of course understood that the RNA sequence may (and frequently does) comprise uracil (U) in place of T. For instance, the RNA sequence may comprise U at every position shown as T in the sequence in Table 12. More specifically, the present disclosure provides an RNA sequence according to every gRNA scaffold sequence of Table 12, wherein the RNA sequence has a U in place of each T in the sequence in Table 12. Additionally, it is understood that terminal Us and Ts may optionally be added or removed from tracrRNA sequences and may be modified or unmodified when provided as RNA. Without wishing to be bound by example, versions of gRNA scaffold sequences alternative to those exemplified in Table 12 may also function with the different Cas9 enzymes or derivatives thereof exemplified in Table 8, e.g., alternate gRNA scaffold sequences with nucleotide additions, substitutions, or deletions, e.g., sequences with stem-loop structures added or removed. It is contemplated herein that the gRNA scaffold sequences represent a component of heterologous gene modifying systems that can be similarly optimized for a given system, Cas-RT fusion polypeptide, indication, target mutation, template RNA, or delivery vehicle.

### Heterologous Object Sequence

A template RNA described herein may comprise a heterologous object sequence that the heterologous gene modifying polypeptide can use as a template for reverse transcription, to write a desired sequence into the target nucleic acid. In some embodiments, the heterologous object sequence comprises, from 5′ to 3′, a post-edit homology region, the mutation region, and a pre-edit homology region. Without wishing to be bound by theory, an RT performing reverse transcription on the template RNA first reverse transcribes the pre-edit homology region, then the mutation region, and then the post-edit homology region, thereby creating a DNA strand comprising the desired mutation with a homology region on either side.

In some embodiments, the heterologous object sequence is at least 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 120, 140, 160, 180, 200, 500, or 1,000 nucleotides (nts) in length, or at least 1, 1.5, 2, 2.5, 3, 3.5, 4, 4.5, 5, 5.5, 6, 6.5, 7, 7.5, 8, 8.5, 9, 9.5, or 10 kilobases in length. In some embodiments, the heterologous object sequence is no more than 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 120, 140, 160, 180, 200, 500, 1,000, or 2000 nucleotides (nts) in length, or no more than 20, 15, 10, 9, 8, 7, 6, 5, 4, or 3 kilobases in length. In some embodiments, the heterologous object sequence is 30-1000, 40-1000, 50-1000, 60-1000, 70-1000, 74-1000, 75-1000, 76-1000, 77-1000, 78-1000, 79-1000, 80-1000, 85-1000, 90-1000, 100-1000, 120-1000, 140-1000, 160-1000, 180-1000, 200-1000, 500-1000, 30-500, 40-500, 50-500, 60-500, 70-500, 74-500, 75-500, 76-500, 77-500, 78-500, 79-500, 80-500, 85-500, 90-500, 100-500, 120-500, 140-500, 160-500, 180-500, 200-500, 30-200, 40-200, 50-200, 60-200, 70-200, 74-200, 75-200, 76-200, 77-200, 78-200, 79-200, 80-200, 85-200, 90-200, 100-200, 120-200, 140-200, 160-200, 180-200, 30-100, 40-100, 50-100, 60-100, 70-100, 74-100, 75-100, 76-100, 77-100, 78-100, 79-100, 80-100, 85-100, or 90-100 nucleotides (nts) in length, or 1-20, 1-15, 1-10, 1-9, 1-8, 1-7, 1-6, 1-5, 1-4, 1-3, 1-2, 2-20, 2-15, 2-10, 2-9, 2-8, 2-7, 2-6, 2-5, 2-4, 2-3, 3-20, 3-15, 3-10, 3-9, 3-8, 3-7, 3-6, 3-5, 3-4, 4-20, 4-15, 4-10, 4-9, 4-8, 4-7, 4-6, 4-5, 5-20, 5-15, 5-10, 5-9, 5-8, 5-7, 5-6, 6-20, 6-15, 6-10, 6-9, 6-8, 6-7, 7-20, 7-15, 7-10, 7-9, 7-8, 8-20, 8-15, 8-10, 8-9, 9-20, 9-15, 9-10, 10-15, 10-20, or 15-20 kilobases in length. In some embodiments, the heterologous object sequence is 10-100, 10-90, 10-80, 10-70, 10-60, 10-50, 10-40, 10-30, or 10-20 nt in length, e.g., 10-80, 10-50, or 10-20 nt in length, e.g., about 10-20 nt in length. In some embodiments, the heterologous object sequence is 8-30, 9-25, 10-20, 11-16, or 12-15 nucleotides in length, e.g., is 11-16 nt in length. Without wishing to be bound by theory, in some embodiments, a larger insertion size, larger region of editing (e.g., the distance between a first edit/substitution and a second edit/substitution in the target region), and/or greater number of desired edits (e.g., mismatches of the heterologous object sequence to the target genome), may result in a longer optimal heterologous object sequence.

In certain embodiments, the template nucleic acid comprises a customized RNA sequence template which can be identified, designed, engineered and constructed to contain sequences altering or specifying host genome function, for example by introducing a heterologous coding region into a genome; affecting or causing exon structure/alternative splicing, e.g., leading to exon skipping of one or more exons; causing disruption of an endogenous gene, e.g., creating a genetic knockout; causing transcriptional activation of an endogenous gene; causing epigenetic regulation of an endogenous DNA; causing up-regulation of one or more operably linked genes, e.g., leading to gene activation or overexpression; causing down-regulation of one or more operably linked genes, e.g., creating a genetic knock-down; etc. In certain embodiments, a customized RNA sequence template can be engineered to contain sequences coding for exons and/or transgenes, provide binding sites for transcription factor activators, repressors, enhancers, etc., and combinations thereof. In some embodiments, a customized template can be engineered to encode a nucleic acid or peptide tag to be expressed in an endogenous RNA transcript or endogenous protein operably linked to the target site. In other embodiments, the coding sequence can be further customized with splice donor sites, splice acceptor sites, or poly-A tails.

The template nucleic acid (e.g., template RNA) of the system typically comprises an object sequence (e.g., a heterologous object sequence) for writing a desired sequence into a target DNA. The object sequence may be coding or non-coding. The template nucleic acid (e.g., template RNA) can be designed to result in insertions, mutations, or deletions at the target DNA locus. In some embodiments, the template nucleic acid (e.g., template RNA) may be designed to cause an insertion in the target DNA. For example, the template nucleic acid (e.g., template RNA) may contain a heterologous sequence, wherein the reverse transcription will result in insertion of the heterologous sequence into the target DNA. In other embodiments, the RNA template may be designed to introduce a deletion into the target DNA. For example, the template nucleic acid (e.g., template RNA) may match the target DNA upstream and downstream of the desired deletion, wherein the reverse transcription will result in the copying of the upstream and downstream sequences from the template nucleic acid (e.g., template RNA) without the intervening sequence, e.g., causing deletion of the intervening sequence. In other embodiments, the template nucleic acid (e.g., template RNA) may be designed to introduce an edit into the target DNA. For example, the template RNA may match the target DNA sequence with the exception of one or more nucleotides, wherein the reverse transcription will result in the copying of these edits into the target DNA, e.g., resulting in mutations, e.g., transition or transversion mutations.

In some embodiments, writing of an object sequence into a target site results in the substitution of nucleotides, e.g., where the full length of the object sequence corresponds to a matching length of the target site with one or more mismatched bases. In some embodiments, a heterologous object sequence may be designed such that a combination of sequence alterations may occur, e.g., a simultaneous addition and deletion, addition and substitution, or deletion and substitution.

In some embodiments, the heterologous object sequence may contain an open reading frame or a fragment of an open reading frame. In some embodiments the heterologous object sequence has a Kozak sequence. In some embodiments the heterologous object sequence has an internal ribosome entry site. In some embodiments the heterologous object sequence has a self-cleaving peptide such as a T2A or P2A site. In some embodiments the heterologous object sequence has a start codon. In some embodiments the template RNA has a splice acceptor site. In some embodiments the template RNA has a splice donor site. Exemplary splice acceptor and splice donor sites are described in WO2016044416, incorporated herein by reference in its entirety. Exemplary splice acceptor site sequences are known to those of skill in the art. In some embodiments the template RNA has a microRNA binding site downstream of the stop codon. In some embodiments the template RNA has a poly A tail downstream of the stop codon of an open reading frame. In some embodiments the template RNA comprises one or more exons. In some embodiments the template RNA comprises one or more introns. In some embodiments the template RNA comprises a eukaryotic transcriptional terminator. In some embodiments the template RNA comprises an enhanced translation element or a translation enhancing element. In some embodiments the RNA comprises the human T-cell leukemia virus (HTLV-1) R region. In some embodiments the RNA comprises a posttranscriptional regulatory element that enhances nuclear export, such as that of Hepatitis B Virus (HPRE) or Woodchuck Hepatitis Virus (WPRE).

In some embodiments, the heterologous object sequence may contain a non-coding sequence. For example, the template nucleic acid (e.g., template RNA) may comprise a regulatory element, e.g., a promoter or enhancer sequence or miRNA binding site. In some embodiments, integration of the object sequence at a target site will result in upregulation of an endogenous gene. In some embodiments, integration of the object sequence at a target site will result in downregulation of an endogenous gene. In some embodiments the template nucleic acid (e.g., template RNA) comprises a tissue specific promoter or enhancer, each of which may be unidirectional or bidirectional. In some embodiments the promoter is an RNA polymerase I promoter, RNA polymerase II promoter, or RNA polymerase III promoter. In some embodiments the promoter comprises a TATA element. In some embodiments the promoter comprises a B recognition element. In some embodiments the promoter has one or more binding sites for transcription factors.

In some embodiments, the template nucleic acid (e.g., template RNA) comprises a site that coordinates epigenetic modification. In some embodiments, the template nucleic acid (e.g., template RNA) comprises a chromatin insulator. For example, the template nucleic acid (e.g., template RNA) comprises a CTCF site or a site targeted for DNA methylation.

In some embodiments, the template nucleic acid (e.g., template RNA) comprises a gene expression unit composed of at least one regulatory region operably linked to an effector sequence. The effector sequence may be a sequence that is transcribed into RNA (e.g., a coding sequence or a non-coding sequence such as a sequence encoding a micro RNA).

In some embodiments, the heterologous object sequence of the template nucleic acid (e.g., template RNA) is inserted into a target genome in an endogenous intron. In some embodiments, the heterologous object sequence of the template nucleic acid (e.g., template RNA) is inserted into a target genome and thereby acts as a new exon. In some embodiments, the insertion of the heterologous object sequence into the target genome results in replacement of a natural exon or the skipping of a natural exon.

The template nucleic acid (e.g., template RNA) can be designed to result in insertions, mutations, or deletions at the target DNA locus. In some embodiments, the template nucleic acid (e.g., template RNA) may be designed to cause an insertion in the target DNA. For example, the template nucleic acid (e.g., template RNA) may contain a heterologous object sequence, wherein the reverse transcription will result in insertion of the heterologous object sequence into the target DNA. In other embodiments, the RNA template may be designed to write a deletion into the target DNA. For example, the template nucleic acid (e.g., template RNA) may match the target DNA upstream and downstream of the desired deletion, wherein the reverse transcription will result in the copying of the upstream and downstream sequences from the template nucleic acid (e.g., template RNA) without the intervening sequence, e.g., causing deletion of the intervening sequence. In other embodiments, the template nucleic acid (e.g., template RNA) may be designed to write an edit into the target DNA. For example, the template RNA may match the target DNA sequence with the exception of one or more nucleotides, wherein the reverse transcription will result in the copying of these edits into the target DNA, e.g., resulting in mutations, e.g., transition or transversion mutations.

In some embodiments, the pre-edit homology domain comprises a nucleic acid sequence having 100% sequence identity with a nucleic acid sequence comprised in a target nucleic acid molecule.

In some embodiments, the post-edit homology domain comprises a nucleic acid sequence having 100% sequence identity with a nucleic acid sequence comprised in a target nucleic acid molecule.

### PBS Sequence

In some embodiments, a template nucleic acid (e.g., template RNA) comprises a PBS sequence. In some embodiments, a PBS sequence is disposed 3′ of the heterologous object sequence and is complementary to a sequence adjacent to a site to be modified by a system described herein, or comprises no more than 1, 2, 3, 4, or 5 mismatches to a sequence complementary to the sequence adjacent to a site to be modified by the system/heterologous gene modifying polypeptide. In some embodiments, the PBS sequence binds within 1, 2, 3, 4, 5, 6, 7, 8, 9, or 10 nucleotides of a nick site in the target nucleic acid molecule. In some embodiments, binding of the PBS sequence to the target nucleic acid molecule permits initiation of target-primed reverse transcription (TPRT), e.g., with the 3′ homology domain acting as a primer for TPRT. In some embodiments, the PBS sequence is 3-5, 5-10, 10-30, 10-25, 10-20, 10-19, 10-18, 10-17, 10-16, 10-15, 10-14, 10-13, 10-12, 10-11, 11-30, 11-25, 11-20, 11-19, 11-18, 11-17, 11-16, 11-15, 11-14, 11-13, 11-12, 12-30, 12-25, 12-20, 12-19, 12-18, 12-17, 12-16, 12-15, 12-14, 12-13, 13-30, 13-25, 13-20, 13-19, 13-18, 13-17, 13-16, 13-15, 13-14, 14-30, 14-25, 14-20, 14-19, 14-18, 14-17, 14-16, 14-15, 15-30, 15-25, 15-20, 15-19, 15-18, 15-17, 15-16, 16-30, 16-25, 16-20, 16-19, 16-18, 16-17, 17-30, 17-25, 17-20, 17-19, 17-18, 18-30, 18-25, 18-20, 18-19, 19-30, 19-25, 19-20, 20-30, 20-25, or 25-30 nucleotides in length, e.g., 10-17, 12-16, or 12-14 nucleotides in length. In some embodiments, the PBS sequence is 5-20, 8-16, 8-14, 8-13, 9-13, 9-12, or 10-12 nucleotides in length, e.g., 9-13 nucleotides in length.

The template nucleic acid (e.g., template RNA) may have some homology to the target DNA. In some embodiments, the template nucleic acid (e.g., template RNA) PBS sequence domain may serve as an annealing region to the target DNA, such that the target DNA is positioned to prime the reverse transcription of the template nucleic acid (e.g., template RNA). In some embodiments the template nucleic acid (e.g., template RNA) has at least 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 175, 200 or more bases of exact homology to the target DNA at the 3′ end of the RNA. In some embodiments the template nucleic acid (e.g., template RNA) has at least 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 175, 200 or more bases of at least 50%, 60%, 70%, 80%, 85%, 90%, 95%, 97%, 98%, 99% or 100% homology to the target DNA, e.g., at the 5′ end of the template nucleic acid (e.g., template RNA).

### Exemplary Template Sequences

A template RNA described herein may have a geometry configured as described herein.

For example, the template may comprise, e.g., from 5′ to 3′:
(i) a gRNA spacer that is complementary to a first portion of a target DNA;
(ii) a gRNA scaffold;
(iii) a heterologous object sequence comprising a mutation region (wherein optionally the heterologous object sequence comprises, from 5′ to 3′, a post-edit homology region, a mutation region, and a pre-edit homology region), and
(iv) a primer binding site (PBS) sequence.

In some embodiments, the mutation region comprises 5 nucleotides or less, e.g., 1, 2, 3, 4, or 5 nucleotides. In some embodiments, the mutation region is configured to produce an insertion. In some embodiments, the mutation region contains 1, 2, 3, 4, or 5 nucleotides that do not have corresponding positions in the target DNA.

In some embodiments, the template RNA is configured to install a mutation close to the nick site in the target DNA. Consequently, in some embodiments, the template RNA does not comprise a pre-edit homology region. In some embodiments, the pre-edit homology region is less than 3, 2, or 1 nucleotides. In some embodiments, the template RNA introduces the mutation at the protospacer nick site. In some embodiments, after installation of the mutation, there is less than 3, 2, or 1 nucleotides between the mutation and the site where the protospacer nick was made. In some embodiments, the template RNA does not introduce the mutation into the PAM location associated with the protospacer.

A template RNA described herein may have a geometry configured as described herein.

For example, the template may comprise, e.g., from 5′ to 3′:
(i) a gRNA spacer that is complementary to a first portion of a target DNA;
(ii) a gRNA scaffold;
(iii) a heterologous object sequence comprising a mutation region (wherein optionally the heterologous object sequence comprises, from 5′ to 3′, a post-edit homology region, a mutation region, and a pre-edit homology region), and
(iv) a primer binding site (PBS) sequence.

In some embodiments, the mutation region comprises 5 nucleotides or less, e.g., 1, 2, 3, 4, or 5 nucleotides. In some embodiments, the mutation region is configured to produce an insertion. In some embodiments, the mutation region contains 1, 2, 3, 4, or 5 nucleotides that do not have corresponding positions in the target DNA.

In some embodiments, the template RNA is configured to install a mutation close to the nick site in the target DNA. Consequently, in some embodiments, the template RNA does not comprise a pre-edit homology region. In some embodiments, the pre-edit homology region is less than 3, 2, or 1 nucleotides. In some embodiments, the template RNA introduces the mutation at the protospacer nick site. In some embodiments, after installation of the mutation, there is less than 3, 2, or 1 nucleotides between the mutation and the site where the protospacer nick was made. In some embodiments, the template RNA does not introduce the mutation into the PAM location associated with the protospacer.

For example, the template may comprise, e.g., from 5′ to 3′:
(i) a gRNA spacer that is complementary to a first portion of a target DNA;
(ii) a gRNA scaffold;
(iii) a heterologous object sequence comprising a mutation region configured to make an insertion of between 1 and 5 nucleotides in the target DNA (wherein optionally the heterologous object sequence comprises, from 5′ to 3′, a post-edit homology region, a mutation region, and a pre-edit homology region), and
(iv) a primer binding site (PBS) sequence.

In some embodiments, a pre-edit homology region is not present, or is present and has a length of no more than 1 or 2 nucleotides.

In some embodiments, the template is designed to introduce the mutation at the protospacer nick site.

In some embodiments of the systems and methods herein, the template RNA comprises a gRNA spacer comprising the nucleotides of a gRNA spacer sequence of Table 1A or 1B. In some embodiments of the systems and methods herein, the heterologous object sequence comprises the nucleotides of an RT template sequence from Table 1A or 1B. In some embodiments, the heterologous object sequence comprises the nucleotides of the RT template sequence of Table 3A or 3B that corresponds to the gRNA spacer sequence. In the context of the sequence tables, a first component “corresponds to” a second component when both components are found in the same row of the table (e.g., in the same cell or in different cells of the same row). In some embodiments, the primer binding site (PBS) sequence has a sequence comprising the nucleotides of a PBS sequence from the same row of Table 1A or 1B as the RT template sequence. In some embodiments, the template RNA comprising a sequence of Table 1A or 1B is comprised by a system that further comprises a heterologous gene modifying polypeptide having an RT domain. RT domain amino acid sequences can be found, e.g., in Table 6 herein.

TABLE 1A
Exemplary Template RNAs and Sequences
Table 1A provides exemplary template RNAs for use in combination within
a heterologous gene modifying system described herein for disrupting,
the TRAC gene using a TRAC4 gRNA spacer (gagaaucaaaaucggugaau SEQ ID
NO. 17513) or a TRAC5 gRNA spacer (gcugguacacggcaggguca SEQ ID NO. 17514).
The description of each exemplary template RNA (column 2) identifies the gRNA
spacer, the length of the PBS, the length of the heterologous object sequence,
the nature and size of the edit, and the location in the target nucleic acid
sequence. For example TRAC4_P13R10_ins5-nick indicates that the template RNA
comprised a TRAC4 gRNA spacer, a PBS of length 13 nucleotides, an RT region
(heterologous object sequence and insertion/deletion) of length 10 nucleotides,
and was designed to produce an insertion of 5 nucleotides near the nick. In
the sequences of Table 1A, the gRNA spacer is the 5′-most sequence and has
the sequence of TRAC4 or TRAC5; immediately 3′of the gRNA spacer is the
gRNA scaffold which comprises a sequence of SEQ ID NO: 17515; immediately 3′
of the gRNA scaffold is the heterologous object sequence which has the
length indicated in the description of column 2; and immediately 3′of
the heterologous sequence is the PBS which has the length indicated in
the description of column 2. Nucleotide modifications are noted as follows:
phosphorothioate linkages denoted by an asterisk, 2′-O-methyl groups
denoted by an ‘m’ preceding a nucleotide.

| | | | SEQ |
|---|---|---|---|
| | | | ID |
| Identifier | Description | Sequence | NO |
| RNACS5602 | TRAC4_ | mG*mA*mG*rArArUrCrArArArArUrCrGrGrUrGrArArUrGrUrUrUrUrA | 16001 |
| | P13R10_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrArUrUrUrArGrUrGrCrAr | |
| | | CrCrGrArUrUrUrU*mG*mA*mU | |
| RNACS5603 | TRAC4_ | mG*mA*mG*rArArUrCrArArArArUrCrGrGrUrGrArArUrGrUrUrUrUrA | 16002 |
| | P12R10_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrArUrUrUrArGrUrGrCrAr | |
| | | CrCrGrArUrUrU*mU*mG*mA | |
| RNACS5604 | TRAC4_ | mG*mA*mG*rArArUrCrArArArArUrCrGrGrUrGrArArUrGrUrUrUrUrA | 16003 |
| | P11R10_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrArUrUrUrArGrUrGrCrAr | |
| | | CrCrGrArUrU*mU*mU*mG | |
| RNACS5605 | TRAC4_ | mG*mA*mG*rArArUrCrArArArArUrCrGrGrUrGrArArUrGrUrUrUrUrA | 16004 |
| | P10R10_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrArUrUrUrArGrUrGrCrAr | |
| | | CrCrGrArU*mU*mU*mU | |
| RNACS5606 | TRAC4_ | mG*mA*mG*rArArUrCrArArArArUrCrGrGrUrGrArArUrGrUrUrUrUrA | 16005 |
| | P9R10_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrArUrUrUrArGrUrGrCrAr | |
| | | CrCrGrA*mU*mU*mU | |
| RNACS5607 | TRAC4_ | mG*mA*mG*rArArUrCrArArArArUrCrGrGrUrGrArArUrGrUrUrUrUrA | 16006 |
| | P8R10_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrArUrUrUrArGrUrGrCrAr | |
| | | CrCrG*mA*mU*mU | |
| RNACS5608 | TRAC4_ | mG*mA*mG*rArArUrCrArArArArUrCrGrGrUrGrArArUrGrUrUrUrUrA | 16007 |
| | P13R12_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrCrCrUrArUrUrUrArGrUrGr | |
| | | CrArCrCrGrArUrUrUrU*mG*mA*mU | |
| RNACS5609 | TRAC4_ | mG*mA*mG*rArArUrCrArArArArUrCrGrGrUrGrArArUrGrUrUrUrUrA | 16008 |
| | P12R12_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrCrCrUrArUrUrUrArGrUrGr | |
| | | CrArCrCrGrArUrUrU*mU*mG*mA | |
| RNACS5610 | TRAC4_ | mG*mA*mG*rArArUrCrArArArArUrCrGrGrUrGrArArUrGrUrUrUrUrA | 16009 |
| | P11R12_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrCrCrUrArUrUrUrArGrUrGr | |
| | | CrArCrCrGrArUrU*mU*mU*mG | |
| RNACS5611 | TRAC4_ | mG*mA*mG*rArArUrCrArArArArUrCrGrGrUrGrArArUrGrUrUrUrUrA | 16010 |
| | P10R12_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrCrCrUrArUrUrUrArGrUrGr | |
| | | CrArCrCrGrArU*mU*mU*mU | |
| RNACS5612 | TRAC4_ | mG*mA*mG*rArArUrCrArArArArUrCrGrGrUrGrArArUrGrUrUrUrUrA | 16011 |
| | P9R12_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrCrCrUrArUrUrUrArGrUrGr | |
| | | CrArCrCrGrA*mU*mU*mU | |
| RNACS5613 | TRAC4_ | mUmAmGmCrArArGrUrUrArArArArUrArArGrGrCrUrArGrUrCrCrGrUr | 16012 |
| | P8R12_ | UrArUrCrAmAmCmUmUmGmAmAmAmAmAmGmUmGmGmCmAmCmCmGmAmGmUmC | |
| | ins5-nick | mGmGmUmGmCrGrCrCrUrArUrUrUrArGrUrGrCrArCrCrG*mA*mU*mU | |
| | | mG*mA*mG*rArArUrCrArArArArUrCrGrGrUrGrArArUrGrUrUrUrUrA | |
| | | rGrAmGmCmUmAmGmAmAmA | |
| RNACS5614 | TRAC4_ | mG*mA*mG*rArArUrCrArArArArUrCrGrGrUrGrArArUrGrUrUrUrUrA | 16013 |
| | P13R14_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrGrCrCrUrArUrUrUrArGr | |
| | | UrGrCrArCrCrGrArUrUrUrU*mG*mA*mU | |
| RNACS5615 | TRAC4_ | mG*mA*mG*rArArUrCrArArArArUrCrGrGrUrGrArArUrGrUrUrUrUrA | 16014 |
| | P12R14_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrGrCrCrUrArUrUrUrArGr | |
| | | UrGrCrArCrCrGrArUrUrU*mU*mG*mA | |
| RNACS5616 | TRAC4_ | mG*mA*mG*rArArUrCrArArArArUrCrGrGrUrGrArArUrGrUrUrUrUrA | 16015 |
| | P11R14_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrGrCrCrUrArUrUrUrArGr | |
| | | UrGrCrArCrCrGrArUrU*mU*mU*mG | |
| RNACS5617 | TRAC4_ | mG*mA*mG*rArArUrCrArArArArUrCrGrGrUrGrArArUrGrUrUrUrUrA | 16016 |
| | P10R14_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrGrCrCrUrArUrUrUrArGr | |
| | | UrGrCrArCrCrGrArU*mU*mU*mU | |
| RNACS5618 | TRAC4_ | mG*mA*mG*rArArUrCrArArArArUrCrGrGrUrGrArArUrGrUrUrUrUrA | 16017 |
| | P9R14_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrGrCrCrUrArUrUrUrArGr | |
| | | UrGrCrArCrCrGrA*mU*mU*mU | |
| RNACS5619 | TRAC4_ | mG*mA*mG*rArArUrCrArArArArUrCrGrGrUrGrArArUrGrUrUrUrUrA | 16018 |
| | P8R14_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrGrCrCrUrArUrUrUrArGr | |
| | | UrGrCrArCrCrG*mA*mU*mU | |
| RNACS5620 | TRAC4_ | mG*mA*mG*rArArUrCrArArArArUrCrGrGrUrGrArArUrGrUrUrUrUrA | 16019 |
| | P13R16_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrUrCrUrGrCrCrUrArUrUrUr | |
| | | ArGrUrGrCrArCrCrGrArUrUrUrU*mG*mA*mU | |
| RNACS5621 | TRAC4_ | mG*mA*mG*rArArUrCrArArArArUrCrGrGrUrGrArArUrGrUrUrUrUrA | 16020 |
| | P12R16_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrUrCrUrGrCrCrUrArUrUrUr | |
| | | ArGrUrGrCrArCrCrGrArUrUrU*mUmG*mA | |
| RNACS5622 | TRAC4_ | mG*mA*mG*rArArUrCrArArArArUrCrGrGrUrGrArArUrGrUrUrUrUrA | 16021 |
| | P11R16_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrUrCrUrGrCrCrUrArUrUrUr | |
| | | ArGrUrGrCrArCrCrGrArUrU*mU*mU*mG | |
| RNACS5623 | TRAC4_ | mG*mA*mG*rArArUrCrArArArArUrCrGrGrUrGrArArUrGrUrUrUrUrA | 16022 |
| | P10R16_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrUrCrUrGrCrCrUrArUrUrUr | |
| | | ArGrUrGrCrArCrCrGrArU*mU*mU*mU | |
| RNACS5624 | TRAC4_ | mG*mA*mG*rArArUrCrArArArArUrCrGrGrUrGrArArUrGrUrUrUrUrA | 16023 |
| | P9R16_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrUrCrUrGrCrCrUrArUrUrUr | |
| | | ArGrUrGrCrArCrCrGrA*mU*mU*mU | |
| RNACS5625 | TRAC4_ | mG*mA*mG*rArArUrCrArArArArUrCrGrGrUrGrArArUrGrUrUrUrUrA | 16024 |
| | P8R16_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrUrCrUrGrCrCrUrArUrUrUr | |
| | | ArGrUrGrCrArCrCrG*mA*mU*mU | |
| RNACS5626 | TRAC4_ | mG*mA*mG*rArArUrCrArArArArUrCrGrGrUrGrArArUrGrUrUrUrUrA | 16025 |
| | P13R18_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrGrUrCrUrGrCrCrUrArUr | |
| | | UrUrArGrUrGrCrArCrCrGrArUrUrUrU*mG*mA*mU | |
| RNACS5627 | TRAC4_ | mG*mA*mG*rArArUrCrArArArArUrCrGrGrUrGrArArUrGrUrUrUrUrA | 16026 |
| | P12R18_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrGrUrCrUrGrCrCrUrArUr | |
| | | UrUrArGrUrGrCrArCrCrGrArUrUrU*mU*mG*mA | |
| RNACS5628 | TRAC4_ | mG*mA*mG*rArArUrCrArArArArUrCrGrGrUrGrArArUrGrUrUrUrUrA | 16027 |
| | P11R18_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrGrUrCrUrGrCrCrUrArUr | |
| | | UrUrArGrUrGrCrArCrCrGrArUrU*mU*mU*mG | |
| RNACS5629 | TRAC4_ | mG*mA*mG*rArArUrCrArArArArUrCrGrGrUrGrArArUrGrUrUrUrUrA | 16028 |
| | P10R18_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrGrUrCrUrGrCrCrUrArUr | |
| | | UrUrArGrUrGrCrArCrCrGrArU*mU*mU*mU | |
| RNACS5630 | TRAC4_ | mG*mA*mG*rArArUrCrArArArArUrCrGrGrUrGrArArUrGrUrUrUrUrA | 16029 |
| | P9R18_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrUrGrUrCrUrGrCrCrUrArUr | |
| | | UrUrArGrUrGrCrArCrCrGrA*mU*mU*mU | |
| RNACS5631 | TRAC4_ | mG*mA*mG*rArArUrCrArArArArUrCrGrGrUrGrArArUrGrUrUrUrUrA | 16030 |
| | P8R18_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrUrGrUrCrUrGrCrCrUrArUr | |
| | | UrUrArGrUrGrCrArCrCrG*mA*mU*mU | |
| RNACS5632 | TRAC4_ | mG*mA*mG*rArArUrCrArArArArUrCrGrGrUrGrArArUrGrUrUrUrUrA | 16031 |
| | P13R20_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrUrCrUrGrUrCrUrGrCrCrUr | |
| | | ArUrUrUrArGrUrGrCrArCrCrGrArUrUrUrU*mG*mA*mU | |
| RNACS5633 | TRAC4_ | mG*mA*mG*rArArUrCrArArArArUrCrGrGrUrGrArArUrGrUrUrUrUrA | 16032 |
| | P12R20_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrUrCrUrGrUrCrUrGrCrCrUr | |
| | | ArUrUrUrArGrUrGrCrArCrCrGrArUrUrUmU*mG*mA | |
| RNACS5634 | TRAC4_ | mG*mA*mG*rArArUrCrArArArArUrCrGrGrUrGrArArUrGrUrUrUrUrA | 16033 |
| | P11R20_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrUrCrUrGrUrCrUrGrCrCrUr | |
| | | ArUrUrUrArGrUrGrCrArCrCrGrArUrU*mU*mU*mG | |
| RNACS5635 | TRAC4_ | mG*mA*mG*rArArUrCrArArArArUrCrGrGrUrGrArArUrGrUrUrUrUrA | 16034 |
| | P10R20_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrUrCrUrGrUrCrUrGrCrCrUr | |
| | | ArUrUrUrArGrUrGrCrArCrCrGrArU*mU*mU*mU | |
| RNACS5636 | TRAC4_ | mG*mA*mG*rArArUrCrArArArArUrCrGrGrUrGrArArUrGrUrUrUrUrA | 16035 |
| | P9R20_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrUrCrUrGrUrCrUrGrCrCrUr | |
| | | ArUrUrUrArGrUrGrCrArCrCrGrA*mU*mU*mU | |
| RNACS5637 | TRAC4_ | mG*mA*mG*rArArUrCrArArArArUrCrGrGrUrGrArArUrGrUrUrUrUrA | 16036 |
| | P8R20_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrUrCrUrGrUrCrUrGrCrCrUr | |
| | | ArUrUrUrArGrUrGrCrArCrCrG*mA*mU*mU | |
| RNACS5638 | TRAC4_ | mG*mA*mG*rArArUrCrArArArArUrCrGrGrUrGrArArUrGrUrUrUrUrA | 16037 |
| | P13R22_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArArGrUrCrUrGrUrCrUrGrCr | |
| | | CrUrArUrUrUrArGrUrGrCrArCrCrGrArUrUrUrU*mG*mA*mU | |
| RNACS5639 | TRAC4_ | mG*mA*mG*rArArUrCrArArArArUrCrGrGrUrGrArArUrGrUrUrUrUrA | 16038 |
| | P12R22_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrArArGrUrCrUrGrUrCrUrGrCr | |
| | | CrUrArUrUrUrArGrUrGrCrArCrCrGrArUrUrU*mU*mG*mA | |
| RNACS5640 | TRAC4_ | mG*mA*mG*rArArUrCrArArArArUrCrGrGrUrGrArArUrGrUrUrUrUrA | 16039 |
| | P11R22_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrArArGrUrCrUrGrUrCrUrGrCr | |
| | | CrUrArUrUrUrArGrUrGrCrArCrCrGrArUrU*mU*mU*mG | |
| RNACS5641 | TRAC4_ | mG*mA*mG*rArArUrCrArArArArUrCrGrGrUrGrArArUrGrUrUrUrUrA | 16040 |
| | P10R22_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrArArGrUrCrUrGrUrCrUrGrCr | |
| | | CrUrArUrUrUrArGrUrGrCrArCrCrGrArU*mU*mU*mU | |
| RNACS5642 | TRAC4_ | mG*mA*mG*rArArUrCrArArArArUrCrGrGrUrGrArArUrGrUrUrUrUrA | 16041 |
| | P9R22_ | rGrAmGmCmUmAmGmAmAmAmUmAmGmCrArArGrUrUrArArArArUrArArGr | |
| | ins5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrArArGrUrCrUrGrUrCrUrGrCr | |
| | | CrUrArUrUrUrArGrUrGrCrArCrCrGrA*mU*mU*mU | |
| RNACS5643 | TRAC4_ | mG*mA*mG*rArArUrCrArArArArUrCrGrGrUrGrArArUrGrUrUrUrUrA | 16042 |
| | P8R22_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArArGrUrCrUrGrUrCrUrGrCr | |
| | | CrUrArUrUrUrArGrUrGrCrArCrCrG*mA*mU*mU | |
| RNACS5644 | TRAC4_ | mG*mA*mG*rArArUrCrArArArArUrCrGrGrUrGrArArUrGrUrUrUrUrA | 16043 |
| | P13R12_ | rGrAmGmCmUmAmGmAmAmAmUmAmGmCrArArGrUrUrArArArArUrArArGr | |
| | ins7-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrArUrUrUrArGrUrUrGrAr | |
| | | CrArCrCrGrArUrUrUrU*mG*mA*mU | |
| RNACS5645 | TRAC4_ | mG*mA*mG*rArArUrCrArArArArUrCrGrGrUrGrArArUrGrUrUrUrUrA | 16044 |
| | P12R12_ | rGrAmGmCmUmAmGmAmAmAmUmAmGmCrArArGrUrUrArArArArUrArArGr | |
| | ins7-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrArUrUrUrArGrUrUrGrAr | |
| | | CrArCrCrGrArUrUrU*mU*mG*mA | |
| RNACS5646 | TRAC4_ | mG*mA*mG*rArArUrCrArArArArUrCrGrGrUrGrArArUrGrUrUrUrUrA | 16045 |
| | P11R12_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrUrArUrUrUrArGrUrUrGrAr | |
| | | CrArCrCrGrArUrU*mU*mU*mG | |
| RNACS5647 | TRAC4_ | mG*mA*mG*rArArUrCrArArArArUrCrGrGrUrGrArArUrGrUrUrUrUrA | 16046 |
| | P10R12_ | rGrAmGmCmUmAmGmAmAmAmUmAmGmCrArArGrUrUrArArArArUrArArGr | |
| | ins7-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrArUrUrUrArGrUrUrGrAr | |
| | | CrArCrCrGrArU*mU*mU*mU | |
| RNACS5648 | TRAC4_ | mG*mA*mG*rArArUrCrArArArArUrCrGrGrUrGrArArUrGrUrUrUrUrA | 16047 |
| | P9R12_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrUrArUrUrUrArGrUrUrGrAr | |
| | | CrArCrCrGrA*mU*mU*mU | |
| RNACS5649 | TRAC4_ | mUmAmGmCrArArGrUrUrArArArArUrArArGrGrCrUrArGrUrCrCrGrUr | 16048 |
| | P8R12_ | UrArUrCrAmAmCmUmUmGmAmAmAmAmAmGmUmGmGmCmAmCmCmGmAmGmUmC | |
| | ins7-nick | mGmGmUmGmCrCrUrArUrUrUrArGrUrUrGrArCrArCrCrG*mA*mU*mU | |
| | | mG*mA*mG*rArArUrCrArArArArUrCrGrGrUrGrArArUrGrUrUrUrUrA | |
| | | rGrAmGmCmUmAmGmAmAmA | |
| RNACS5650 | TRAC4_ | mG*mA*mG*rArArUrCrArArArArUrCrGrGrUrGrArArUrGrUrUrUrUrA | 16049 |
| | P13R14_ | rGrAmGmCmUmAmGmAmAmAmUmAmGmCrArArGrUrUrArArArArUrArArGr | |
| | ins7-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrCrCrUrArUrUrUrArGrUrUr | |
| | | GrArCrArCrCrGrArUrUrUrU*mG*mA*mU | |
| RNACS5651 | TRAC4_ | mG*mA*mG*rArArUrCrArArArArUrCrGrGrUrGrArArUrGrUrUrUrUrA | 16050 |
| | P12R14_ | rGrAmGmCmUmAmGmAmAmAmUmAmGmCrArArGrUrUrArArArArUrArArGr | |
| | ins7-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrCrCrUrArUrUrUrArGrUrUr | |
| | | GrArCrArCrCrGrArUrUrU*mU*mG*mA | |
| RNACS5652 | TRAC4_ | mG*mA*mG*rArArUrCrArArArArUrCrGrGrUrGrArArUrGrUrUrUrUrA | 16051 |
| | P11R14_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrCrCrUrArUrUrUrArGrUrUr | |
| | | GrArCrArCrCrGrArUrU*mU*mU*mG | |
| RNACS5653 | TRAC4_ | mG*mA*mG*rArArUrCrArArArArUrCrGrGrUrGrArArUrGrUrUrUrUrA | 16052 |
| | P10R14_ | rGrAmGmCmUmAmGmAmAmAmUmAmGmCrArArGrUrUrArArArArUrArArGr | |
| | ins7-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrCrCrUrArUrUrUrArGrUrUr | |
| | | GrArCrArCrCrGrArU*mU*mU*mU | |
| RNACS5654 | TRAC4_ | mG*mA*mG*rArArUrCrArArArArUrCrGrGrUrGrArArUrGrUrUrUrUrA | 16053 |
| | P9R14_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrCrCrUrArUrUrUrArGrUrUr | |
| | | GrArCrArCrCrGrA*mU*mU*mU | |
| RNACS5655 | TRAC4_ | mG*mA*mG*rArArUrCrArArArArUrCrGrGrUrGrArArUrGrUrUrUrUrA | 16054 |
| | P8R14_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrCrCrUrArUrUrUrArGrUrUr | |
| | | GrArCrArCrCrG*mA*mU*mU | |
| RNACS5656 | TRAC4_ | mG*mA*mG*rArArUrCrArArArArUrCrGrGrUrGrArArUrGrUrUrUrUrA | 16055 |
| | P13R16_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrUrGrCrCrUrArUrUrUrArGr | |
| | | UrUrGrArCrArCrCrGrArUrUrUrU*mG*mA*mU | |
| RNACS5657 | TRAC4_ | mG*mA*mG*rArArUrCrArArArArUrCrGrGrUrGrArArUrGrUrUrUrUrA | 16056 |
| | P12R16_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrUrGrCrCrUrArUrUrUrArGr | |
| | | UrUrGrArCrArCrCrGrArUrUrU*mU*mG*mA | |
| RNACS5658 | TRAC4_ | mG*mA*mG*rArArUrCrArArArArUrCrGrGrUrGrArArUrGrUrUrUrUrA | 16057 |
| | P11R16_ | rGrAmGmCmUmAmGmAmAmAmUmAmGmCrArArGrUrUrArArArArUrArArGr | |
| | ins7-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrGrCrCrUrArUrUrUrArGr | |
| | | UrUrGrArCrArCrCrGrArUrU*mU*mU*mG | |
| RNACS5659 | TRAC4_ | mG*mA*mG*rArArUrCrArArArArUrCrGrGrUrGrArArUrGrUrUrUrUrA | 16058 |
| | P10R16_ | rGrAmGmCmUmAmGmAmAmAmUmAmGmCrArArGrUrUrArArArArUrArArGr | |
| | ins7-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrGrCrCrUrArUrUrUrArGr | |
| | | UrUrGrArCrArCrCrGrArU*mU*mU*mU | |
| RNACS5660 | TRAC4_ | mG*mA*mG*rArArUrCrArArArArUrCrGrGrUrGrArArUrGrUrUrUrUrA | 16059 |
| | P9R16_ | rGrAmGmCmUmAmGmAmAmAmUmAmGmCrArArGrUrUrArArArArUrArArGr | |
| | ins7-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrGrCrCrUrArUrUrUrArGr | |
| | | UrUrGrArCrArCrCrGrA*mU*mU*mU | |
| RNACS5661 | TRAC4_ | mG*mA*mG*rArArUrCrArArArArUrCrGrGrUrGrArArUrGrUrUrUrUrA | 16060 |
| | P8R16_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrUrGrCrCrUrArUrUrUrArGr | |
| | | UrUrGrArCrArCrCrG*mA*mU*mU | |
| RNACS5662 | TRAC4_ | mG*mA*mG*rArArUrCrArArArArUrCrGrGrUrGrArArUrGrUrUrUrUrA | 16061 |
| | P13R18_ | rGrAmGmCmUmAmGmAmAmAmUmAmGmCrArArGrUrUrArArArArUrArArGr | |
| | ins7-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrUrCrUrGrCrCrUrArUrUrUr | |
| | | ArGrUrUrGrArCrArCrCrGrArUrUrUrU*mG*mA*mU | |
| RNACS5663 | TRAC4_ | mG*mA*mG*rArArUrCrArArArArUrCrGrGrUrGrArArUrGrUrUrUrUrA | 16062 |
| | P12R18_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrUrCrUrGrCrCrUrArUrUrUr | |
| | | ArGrUrUrGrArCrArCrCrGrArUrUrU*mU*mG*mA | |
| RNACS5664 | TRAC4_ | mG*mA*mG*rArArUrCrArArArArUrCrGrGrUrGrArArUrGrUrUrUrUrA | 16063 |
| | P11R18_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrUrCrUrGrCrCrUrArUrUrUr | |
| | | ArGrUrUrGrArCrArCrCrGrArUrU*mU*mU*mG | |
| RNACS5665 | TRAC4_ | mG*mA*mG*rArArUrCrArArArArUrCrGrGrUrGrArArUrGrUrUrUrUrA | 16064 |
| | P10R18_ | rGrAmGmCmUmAmGmAmAmAmUmAmGmCrArArGrUrUrArArArArUrArArGr | |
| | ins7-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrUrCrUrGrCrCrUrArUrUrUr | |
| | | ArGrUrUrGrArCrArCrCrGrArU*mU*mU*mU | |
| RNACS5666 | TRAC4_ | mG*mA*mG*rArArUrCrArArArArUrCrGrGrUrGrArArUrGrUrUrUrUrA | 16065 |
| | P9R18_ | rGrAmGmCmUmAmGmAmAmAmUmAmGmCrArArGrUrUrArArArArUrArArGr | |
| | ins7-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrUrCrUrGrCrCrUrArUrUrUr | |
| | | ArGrUrUrGrArCrArCrCrGrA*mU*mU*mU | |
| RNACS5667 | TRAC4_ | mUmAmGmCrArArGrUrUrArArArArUrArArGrGrCrUrArGrUrCrCrGrUr | 16066 |
| | P8R18_ | UrArUrCrAmAmCmUmUmGmAmAmAmAmAmGmUmGmGmCmAmCmCmGmAmGmUmC | |
| | ins7- | mGmGmUmGmCrGrUrCrUrGrCrCrUrArUrUrUrArGrUrUrGrArCrArCr | |
| | nick | CrG*mA*mU*mUmG*mA*mG*rArArUrCrArArArArUrCrGrGrUrGrArArU | |
| | | rGrUrUrUrUrArGrAmGmCmUmAmGmAmAmA | |
| RNACS5668 | TRAC4_ | mG*mA*mG*rArArUrCrArArArArUrCrGrGrUrGrArArUrGrUrUrUrUrA | 16067 |
| | P13R20_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrUrGrUrCrUrGrCrCrUrArUr | |
| | | UrUrArGrUrUrGrArCrArCrCrGrArUrUrUrU*mG*mA*mU | |
| RNACS5669 | TRAC4_ | mG*mA*mG*rArArUrCrArArArArUrCrGrGrUrGrArArUrGrUrUrUrUrA | 16068 |
| | P12R20_ | rGrAmGmCmUmAmGmAmAmAmUmAmGmCrArArGrUrUrArArArArUrArArGr | |
| | ins7-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrGrUrCrUrGrCrCrUrArUr | |
| | | UrUrArGrUrUrGrArCrArCrCrGrArUrUrU*mU*mG*mA | |
| RNACS5670 | TRAC4_ | mG*mA*mG*rArArUrCrArArArArUrCrGrGrUrGrArArUrGrUrUrUrUrA | 16069 |
| | P11R20_ | rGrAmGmCmUmAmGmAmAmAmUmAmGmCrArArGrUrUrArArArArUrArArGr | |
| | ins7-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrGrUrCrUrGrCrCrUrArUr | |
| | | UrUrArGrUrUrGrArCrArCrCrGrArUrU*mU*mU*mG | |
| RNACS5671 | TRAC4_ | mG*mA*mG*rArArUrCrArArArArUrCrGrGrUrGrArArUrGrUrUrUrUrA | 16070 |
| | P10R20_ | rGrAmGmCmUmAmGmAmAmAmUmAmGmCrArArGrUrUrArArArArUrArArGr | |
| | ins7-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrGrUrCrUrGrCrCrUrArUr | |
| | | UrUrArGrUrUrGrArCrArCrCrGrArU*mU*mU*mU | |
| RNACS5672 | TRAC4_ | mG*mA*mG*rArArUrCrArArArArUrCrGrGrUrGrArArUrGrUrUrUrUrA | 16071 |
| | P9R20_ | rGrAmGmCmUmAmGmAmAmAmUmAmGmCrArArGrUrUrArArArArUrArArGr | |
| | ins7-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrGrUrCrUrGrCrCrUrArUr | |
| | | UrUrArGrUrUrGrArCrArCrCrGrA*mU*mU*mU | |
| RNACS5673 | TRAC4_ | mG*mA*mG*rArArUrCrArArArArUrCrGrGrUrGrArArUrGrUrUrUrUrA | 16072 |
| | P8R20_ | rGrAmGmCmUmAmGmAmAmAmUmAmGmCrArArGrUrUrArArArArUrArArGr | |
| | ins7-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrGrUrCrUrGrCrCrUrArUr | |
| | | UrUrArGrUrUrGrArCrArCrCrG*mA*mU*mU | |
| RNACS5674 | TRAC4_ | mG*mA*mG*rArArUrCrArArArArUrCrGrGrUrGrArArUrGrUrUrUrUrA | 16073 |
| | P13R22_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrUrCrUrGrUrCrUrGrCrCrUr | |
| | | ArUrUrUrArGrUrUrGrArCrArCrCrGrArUrUrUrU*mG*mA*mU | |
| RNACS5675 | TRAC4_ | mG*mA*mG*rArArUrCrArArArArUrCrGrGrUrGrArArUrGrUrUrUrUrA | 16074 |
| | P12R22_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrUrCrUrGrUrCrUrGrCrCrUr | |
| | | ArUrUrUrArGrUrUrGrArCrArCrCrGrArUrUrU*mU*mG*mA | |
| RNACS5676 | TRAC4_ | mG*mA*mG*rArArUrCrArArArArUrCrGrGrUrGrArArUrGrUrUrUrUrA | 16075 |
| | P11R22_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrUrCrUrGrUrCrUrGrCrCr | |
| | | UrArUrUrUrArGrUrUrGrArCrArCrCrGrArUrU*mUmU*mG | |
| RNACS5677 | TRAC4_ | mG*mA*mG*rArArUrCrArArArArUrCrGrGrUrGrArArUrGrUrUrUrUrA | 16076 |
| | P10R22_ | rGrAmGmCmUmAmGmAmAmAmUmAmGmCrArArGrUrUrArArArArUrArArGr | |
| | ins7-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrUrCrUrGrUrCrUrGrCrCrUr | |
| | | ArUrUrUrArGrUrUrGrArCrArCrCrGrArU*mU*mU*mU | |
| RNACS5678 | TRAC4_P9R22_ | mG*mA*mG*rArArUrCrArArArArUrCrGrGrUrGrArArUrGrUrUrUrUrA | 16077 |
| | ins7-nick | rGrAmGmCmUmAmGmAmAmAmUmAmGmCrArArGrUrUrArArArArUrArArGr | |
| | | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrUrCrUrGrUrCrUrGrCrCrUr | |
| | | ArUrUrUrArGrUrUrGrArCrArCrCrGrA*mU*mU*mU | |
| RNACS5679 | TRAC4_ | mG*mA*mG*rArArUrCrArArArArUrCrGrGrUrGrArArUrGrUrUrUrUrA | 16078 |
| | P8R22_ | rGrAmGmCmUmAmGmAmAmAmUmAmGmCrArArGrUrUrArArArArUrArArGr | |
| | ins7-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrUrCrUrGrUrCrUrGrCrCrUr | |
| | | ArUrUrUrArGrUrUrGrArCrArCrCrG*mA*mU*mU | |
| RNACS5680 | TRAC4_ | mG*mA*mG*rArArUrCrArArArArUrCrGrGrUrGrArArUrGrUrUrUrUrA | 16079 |
| | P13R24_ | rGrAmGmCmUmAmGmAmAmAmUmAmGmCrArArGrUrUrArArArArUrArArGr | |
| | ins7-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrArArGrUrCrUrGrUrCrUrGrCr | |
| | | CrUrArUrUrUrArGrUrUrGrArCrArCrCrGrArUrUrUrU*mG*mA*mU | |
| RNACS5681 | TRAC4_ | mG*mA*mG*rArArUrCrArArArArUrCrGrGrUrGrArArUrGrUrUrUrUrA | 16080 |
| | P12R24_ | rGrAmGmCmUmAmGmAmAmAmUmAmGmCrArArGrUrUrArArArArUrArArGr | |
| | ins7-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrArArGrUrCrUrGrUrCrUrGrCr | |
| | | CrUrArUrUrUrArGrUrUrGrArCrArCrCrGrArUrUrU*mU*mG*mA | |
| RNACS5682 | TRAC4_ | mG*mA*mG*rArArUrCrArArArArUrCrGrGrUrGrArArUrGrUrUrUrUrA | 16081 |
| | P11R24_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArArGrUrCrUrGrUrCrUrGrCr | |
| | | CrUrArUrUrUrArGrUrUrGrArCrArCrCrGrArUrU*mU*mU*mG | |
| RNACS5683 | TRAC4_ | mG*mA*mG*rArArUrCrArArArArUrCrGrGrUrGrArArUrGrUrUrUrUrA | 16082 |
| | P10R24_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArArGrUrCrUrGrUrCrUrGrCr | |
| | | CrUrArUrUrUrArGrUrUrGrArCrArCrCrGrArU*mU*mU*mU | |
| RNACS5684 | TRAC4_ | mG*mA*mG*rArArUrCrArArArArUrCrGrGrUrGrArArUrGrUrUrUrUrA | 16083 |
| | P9R24_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArArGrUrCrUrGrUrCrUrGrCr | |
| | | CrUrArUrUrUrArGrUrUrGrArCrArCrCrGrA*mU*mU*mU | |
| RNACS5685 | TRAC4_ | mUmAmGmCrArArGrUrUrArArArArUrArArGrGrCrUrArGrUrCrCrGrUr | 16084 |
| | P8R24_ | UrArUrCrAmAmCmUmUmGmAmAmAmAmAmGmUmGmGmCmAmCmCmGmAmGmUmC | |
| | ins7- | mGmGmUmGmCrArArGrUrCrUrGrUrCrUrGrCrCrUrArUrUrUrArGrUr | |
| | nick | UrGrArCrArCrCrG*mA*mU*mUmG*mA*mG*rArArUrCrArArArArUrCrG | |
| | | rGrUrGrArArUrGrUrUrUrUrArGrAmGmCmUmAmGmAmAmA | |
| RNACS5686 | TRAC4_ | mG*mA*mG*rArArUrCrArArArArUrCrGrGrUrGrArArUrGrUrUrUrUrA | 16085 |
| | P13R5_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrCrUrGrCrCrArCrCrGrArUr | |
| | | UrUrU*mG*mA*mU | |
| RNACS5687 | TRAC4_P12R5_ | mG*mA*mG*rArArUrCrArArArArUrCrGrGrUrGrArArUrGrUrUrUrUrA | 16086 |
| | del5-nick | rGrAmGmCmUmAmGmAmAmAmUmAmGmCrArArGrUrUrArArArArUrArArGr | |
| | | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrUrCrUrGrCrCrArCrCrGrArUr | |
| | | UrU*mU*mG*mA | |
| RNACS5688 | TRAC4_ | mG*mA*mG*rArArUrCrArArArArUrCrGrGrUrGrArArUrGrUrUrUrUrA | 16087 |
| | P11R5_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrCrUrGrCrCrArCrCrGrArUr | |
| | | U*mU*mU*mG | |
| RNACS5689 | TRAC4_ | mG*mA*mG*rArArUrCrArArArArUrCrGrGrUrGrArArUrGrUrUrUrUrA | 16088 |
| | P10R5_ | rGrAmGmCmUmAmGmAmAmAmUmAmGmCrArArGrUrUrArArArArUrArArGr | |
| | del5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrUrCrUrGrCrCrArCrCrGrArU* | |
| | | mU*mU*mU | |
| RNACS5690 | TRAC4_P9R5_ | mG*mA*mG*rArArUrCrArArArArUrCrGrGrUrGrArArUrGrUrUrUrUrA | 16089 |
| | del5-nick | rGrAmGmCmUmAmGmAmAmAmUmAmGmCrArArGrUrUrArArArArUrArArGr | |
| | | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrUrCrUrGrCrCrArCrCrGrA*mU | |
| | | *mU*mU | |
| RNACS5691 | TRAC4_ | mG*mA*mG*rArArUrCrArArArArUrCrGrGrUrGrArArUrGrUrUrUrUrA | 16090 |
| | P8R5_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrCrUrGrCrCrArCrCrG*mA*m | |
| | | U*mU | |
| RNACS5692 | TRAC4_ | mG*mA*mG*rArArUrCrArArArArUrCrGrGrUrGrArArUrGrUrUrUrUrA | 16091 |
| | P13R7_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrGrUrCrUrGrCrCrArCrCrGr | |
| | | ArUrUrUrU*mG*mA*mU | |
| RNACS5693 | TRAC4_ | mG*mA*mG*rArArUrCrArArArArUrCrGrGrUrGrArArUrGrUrUrUrUrA | 16092 |
| | P12R7_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrGrUrCrUrGrCrCrArCrCrGr | |
| | | ArUrUrU*mU*mG*mA | |
| RNACS5694 | TRAC4_ | mG*mA*mG*rArArUrCrArArArArUrCrGrGrUrGrArArUrGrUrUrUrUrA | 16093 |
| | P11R7_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrGrUrCrUrGrCrCrArCrCrGr | |
| | | ArUrU*mU*mU*mG | |
| RNACS5695 | TRAC4_ | mG*mA*mG*rArArUrCrArArArArUrCrGrGrUrGrArArUrGrUrUrUrUrA | 16094 |
| | P10R7_ | rGrAmGmCmUmAmGmAmAmAmUmAmGmCrArArGrUrUrArArArArUrArArGr | |
| | del5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrUrGrUrCrUrGrCrCrArCrCrGr | |
| | | ArU*mU*mU*mU | |
| RNACS5696 | TRAC4_ | mG*mA*mG*rArArUrCrArArArArUrCrGrGrUrGrArArUrGrUrUrUrUrA | 16095 |
| | P9R7_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrGrUrCrUrGrCrCrArCrCrGr | |
| | | A*mU*mU*mU | |
| RNACS5697 | TRAC4_ | mG*mA*mG*rArArUrCrArArArArUrCrGrGrUrGrArArUrGrUrUrUrUrA | 16096 |
| | P8R7_ | rGrAmGmCmUmAmGmAmAmAmUmAmGmCrArArGrUrUrArArArArUrArArGr | |
| | del5-nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrUrGrUrCrUrGrCrCrArCrCrG* | |
| | | mA*mU*mU | |
| RNACS5698 | TRAC4_ | mG*mA*mG*rArArUrCrArArArArUrCrGrGrUrGrArArUrGrUrUrUrUrA | 16097 |
| | P13R9_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrCrUrGrUrCrUrGrCrCrArCr | |
| | | CrGrArUrUrUrU*mG*mA*mU | |
| RNACS5699 | TRAC4_ | mG*mA*mG*rArArUrCrArArArArUrCrGrGrUrGrArArUrGrUrUrUrUrA | 16098 |
| | P12R9_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrCrUrGrUrCrUrGrCrCrArCr | |
| | | CrGrArUrUrU*mU*mG*mA | |
| RNACS5700 | TRAC4_ | mG*mA*mG*rArArUrCrArArArArUrCrGrGrUrGrArArUrGrUrUrUrUrA | 16099 |
| | P11R9_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrCrUrGrUrCrUrGrCrCrArCr | |
| | | CrGrArUrU*mU*mU*mG | |
| RNACS5701 | TRAC4_ | mG*mA*mG*rArArUrCrArArArArUrCrGrGrUrGrArArUrGrUrUrUrUrA | 16100 |
| | P10R9_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrCrUrGrUrCrUrGrCrCrArCr | |
| | | CrGrArU*mU*mU*mU | |
| RNACS5702 | TRAC4_ | mG*mA*mG*rArArUrCrArArArArUrCrGrGrUrGrArArUrGrUrUrUrUrA | 16101 |
| | P9R9_del5_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrUrCrUrGrUrCrUrGrCrCrArCr | |
| | | CrGrA*mU*mU*mU | |
| RNACS5703 | TRAC4_ | mG*mA*mG*rArArUrCrArArArArUrCrGrGrUrGrArArUrGrUrUrUrUrA | 16102 |
| | P8R9_del5_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrUrCrUrGrUrCrUrGrCrCrArCr | |
| | | CrG*mA*mU*mU | |
| RNACS5704 | TRAC4_ | mG*mA*mG*rArArUrCrArArArArUrCrGrGrUrGrArArUrGrUrUrUrUrA | 16103 |
| | P13R11_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArGrUrCrUrGrUrCrUrGrCrCr | |
| | | ArCrCrGrArUrUrUrU*mG*mA*mU | |
| RNACS5705 | TRAC4_ | mG*mA*mG*rArArUrCrArArArArUrCrGrGrUrGrArArUrGrUrUrUrUrA | 16104 |
| | P12R11_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArGrUrCrUrGrUrCrUrGrCrCr | |
| | | ArCrCrGrArUrUrU*mU*mG*mA | |
| RNACS5706 | TRAC4_ | mG*mA*mG*rArArUrCrArArArArUrCrGrGrUrGrArArUrGrUrUrUrUrA | 16105 |
| | P11R11_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArGrUrCrUrGrUrCrUrGrCrCr | |
| | | ArCrCrGrArUrU*mU*mU*mG | |
| RNACS5707 | TRAC4_ | mG*mA*mG*rArArUrCrArArArArUrCrGrGrUrGrArArUrGrUrUrUrUrA | 16106 |
| | P10R11_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArGrUrCrUrGrUrCrUrGrCrCr | |
| | | ArCrCrGrArU*mU*mU*mU | |
| RNACS5708 | TRAC4_ | mG*mA*mG*rArArUrCrArArArArUrCrGrGrUrGrArArUrGrUrUrUrUrA | 16107 |
| | P9R11_ | rGrAmGmCmUmAmGmAmAmAmUmAmGmCrArArGrUrUrArArArArUrArArGr | |
| | de15_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArGrUrCrUrGrUrCrUrGrCrCr | |
| | | ArCrCrGrA*mU*mU*mU | |
| RNACS5709 | TRAC4_ | mG*mA*mG*rArArUrCrArArArArUrCrGrGrUrGrArArUrGrUrUrUrUrA | 16108 |
| | P8R11_del5_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrArGrUrCrUrGrUrCrUrGrCrCr | |
| | | ArCrCrG*mA*mU*mU | |
| RNACS5710 | TRAC4_ | mG*mA*mG*rArArUrCrArArArArUrCrGrGrUrGrArArUrGrUrUrUrUrA | 16109 |
| | P13R13_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArArGrUrCrUrGrUrCrUrGr | |
| | | CrCrArCrCrGrArUrUrUrU*mG*mA*mU | |
| RNACS5711 | TRAC4_ | mG*mA*mG*rArArUrCrArArArArUrCrGrGrUrGrArArUrGrUrUrUrUrA | 16110 |
| | P12R13_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArArGrUrCrUrGrUrCrUrGr | |
| | | CrCrArCrCrGrArUrUrU*mU*mG*mA | |
| RNACS5712 | TRAC4_ | mG*mA*mG*rArArUrCrArArArArUrCrGrGrUrGrArArUrGrUrUrUrUrA | 16111 |
| | P11R13_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArArGrUrCrUrGrUrCrUrGr | |
| | | CrCrArCrCrGrArUrU*mU*mU*mG | |
| RNACS5713 | TRAC4_ | mG*mA*mG*rArArUrCrArArArArUrCrGrGrUrGrArArUrGrUrUrUrUrA | 16112 |
| | P10R13_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArArGrUrCrUrGrUrCrUrGr | |
| | | CrCrArCrCrGrArU*mU*mU*mU | |
| RNACS5714 | TRAC4_ | mG*mA*mG*rArArUrCrArArArArUrCrGrGrUrGrArArUrGrUrUrUrUrA | 16113 |
| | P9R13_ | rGrAmGmCmUmAmGmAmAmAmUmAmGmCrArArGrUrUrArArArArUrArArGr | |
| | de15_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArArGrUrCrUrGrUrCrUrGr | |
| | | CrCrArCrCrGrA*mU*mU*mU | |
| RNACS5715 | TRAC4_ | mG*mA*mG*rArArUrCrArArArArUrCrGrGrUrGrArArUrGrUrUrUrUrA | 16114 |
| | P8R13_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArArGrUrCrUrGrUrCrUrGr | |
| | | CrCrArCrCrG*mA*mU*mU | |
| RNACS5716 | TRAC4_ | mG*mA*mG*rArArUrCrArArArArUrCrGrGrUrGrArArUrGrUrUrUrUrA | 16115 |
| | P13R15_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArCrArArGrUrCrUrGrUrCr | |
| | | UrGrCrCrArCrCrGrArUrUrUrU*mG*mA*mU | |
| RNACS5717 | TRAC4_ | mG*mA*mG*rArArUrCrArArArArUrCrGrGrUrGrArArUrGrUrUrUrUrA | 16116 |
| | P12R15_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArCrArArGrUrCrUrGrUrCr | |
| | | UrGrCrCrArCrCrGrArUrUrU*mU*mG*mA | |
| RNACS5718 | TRAC4_ | mG*mA*mG*rArArUrCrArArArArUrCrGrGrUrGrArArUrGrUrUrUrUrA | 16117 |
| | P11R15_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArCrArArGrUrCrUrGrUrCr | |
| | | UrGrCrCrArCrCrGrArUrU*mU*mU*mG | |
| RNACS5719 | TRAC4_ | mG*mA*mG*rArArUrCrArArArArUrCrGrGrUrGrArArUrGrUrUrUrUrA | 16118 |
| | P10R15_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArCrArArGrUrCrUrGrUrCr | |
| | | UrGrCrCrArCrCrGrArU*mU*mU*mU | |
| RNACS5720 | TRAC4_ | mG*mA*mG*rArArUrCrArArArArUrCrGrGrUrGrArArUrGrUrUrUrUrA | 16119 |
| | P9R15_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArCrArArGrUrCrUrGrUrCr | |
| | | UrGrCrCrArCrCrGrA*mU*mU*mU | |
| RNACS5721 | TRAC4_ | mG*mA*mG*rArArUrCrArArArArUrCrGrGrUrGrArArUrGrUrUrUrUrA | 16120 |
| | P8R15_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArCrArArGrUrCrUrGrUrCr | |
| | | UrGrCrCrArCrCrG*mA*mU*mU | |
| RNACS5722 | TRAC4_ | mG*mA*mG*rArArUrCrArArArArUrCrGrGrUrGrArArUrGrUrUrUrUrA | 16121 |
| | P13R17_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrUrGrArCrArArGrUrCrUrGr | |
| | | UrCrUrGrCrCrArCrCrGrArUrUrUrU*mG*mA*mU | |
| RNACS5723 | TRAC4_ | mG*mA*mG*rArArUrCrArArArArUrCrGrGrUrGrArArUrGrUrUrUrUrA | 16122 |
| | P12R17_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrUrGrArCrArArGrUrCrUrGr | |
| | | UrCrUrGrCrCrArCrCrGrArUrUrU*mU*mG*mA | |
| RNACS5724 | TRAC4_ | mG*mA*mG*rArArUrCrArArArArUrCrGrGrUrGrArArUrGrUrUrUrUrA | 16123 |
| | P11R17_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrUrGrArCrArArGrUrCrUrGr | |
| | | UrCrUrGrCrCrArCrCrGrArUrU*mU*mU*mG | |
| RNACS5725 | TRAC4_ | mG*mA*mG*rArArUrCrArArArArUrCrGrGrUrGrArArUrGrUrUrUrUrA | 16124 |
| | P10R17_ | rGrAmGmCmUmAmGmAmAmAmUmAmGmCrArArGrUrUrArArArArUrArArGr | |
| | de15_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrUrGrArCrArArGrUrCrUrGr | |
| | | UrCrUrGrCrCrArCrCrGrArU*mU*mU*mU | |
| RNACS5726 | TRAC4_ | mG*mA*mG*rArArUrCrArArArArUrCrGrGrUrGrArArUrGrUrUrUrUrA | 16125 |
| | P9R17_ | rGrAmGmCmUmAmGmAmAmAmUmAmGmCrArArGrUrUrArArArArUrArArGr | |
| | de15_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrUrGrArCrArArGrUrCrUrGr | |
| | | UrCrUrGrCrCrArCrCrGrA*mU*mU*mU | |
| RNACS5727 | TRAC4_ | mG*mA*mG*rArArUrCrArArArArUrCrGrGrUrGrArArUrGrUrUrUrUrA | 16126 |
| | P8R17_ | rGrAmGmCmUmAmGmAmAmAmUmAmGmCrArArGrUrUrArArArArUrArArGr | |
| | de15_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrUrGrArCrArArGrUrCrUrGr | |
| | | UrCrUrGrCrCrArCrCrG*mA*mU*mU | |
| RNACS5728 | TRAC4_ | mG*mA*mG*rArArUrCrArArArArUrCrGrGrUrGrArArUrGrUrUrUrUrA | 16127 |
| | P13R5_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrGrUrCrUrCrArCrCrGrArUr | |
| | | UrUrUmG*mA*mU | |
| RNACS5729 | TRAC4_ | mG*mA*mG*rArArUrCrArArArArUrCrGrGrUrGrArArUrGrUrUrUrUrA | 16128 |
| | P12R5_del7_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrUrGrUrCrUrCrArCrCrGrArUr | |
| | | UrU*mU*mG*mA | |
| RNACS5730 | TRAC4_ | mG*mA*mG*rArArUrCrArArArArUrCrGrGrUrGrArArUrGrUrUrUrUrA | 16129 |
| | P11R5_del7_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrUrGrUrCrUrCrArCrCrGrArUr | |
| | | U*mU*mU*mG | |
| RNACS5731 | TRAC4_ | mG*mA*mG*rArArUrCrArArArArUrCrGrGrUrGrArArUrGrUrUrUrUrA | 16130 |
| | P10R5_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrGrUrCrUrCrArCrCrGrArU* | |
| | | mU*mU*mU | |
| RNACS5732 | TRAC4_ | mG*mA*mG*rArArUrCrArArArArUrCrGrGrUrGrArArUrGrUrUrUrUrA | 16131 |
| | P9R5_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrGrUrCrUrCrArCrCrGrA*mU | |
| | | *mU*mU | |
| RNACS5733 | TRAC4_ | mG*mA*mG*rArArUrCrArArArArUrCrGrGrUrGrArArUrGrUrUrUrUrA | 16132 |
| | P8R5_del7_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrUrGrUrCrUrCrArCrCrG*mA*m | |
| | | U*mU | |
| RNACS5734 | TRAC4_ | mG*mA*mG*rArArUrCrArArArArUrCrGrGrUrGrArArUrGrUrUrUrUrA | 16133 |
| | P13R7_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrCrUrGrUrCrUrCrArCrCrGr | |
| | | ArUrUrUrU*mG*mA*mU | |
| RNACS5735 | TRAC4_ | mG*mA*mG*rArArUrCrArArArArUrCrGrGrUrGrArArUrGrUrUrUrUrA | 16134 |
| | P12R7_del7_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrUrCrUrGrUrCrUrCrArCrCrGr | |
| | | ArUrUrU*mU*mG*mA | |
| RNACS5736 | TRAC4_ | mG*mA*mG*rArArUrCrArArArArUrCrGrGrUrGrArArUrGrUrUrUrUrA | 16135 |
| | P11R7_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrCrUrGrUrCrUrCrArCrCrGr | |
| | | ArUrU*mU*mU*mG | |
| RNACS5737 | TRAC4_ | mG*mA*mG*rArArUrCrArArArArUrCrGrGrUrGrArArUrGrUrUrUrUrA | 16136 |
| | P10R7_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrCrUrGrUrCrUrCrArCrCrGr | |
| | | ArU*mU*mU*mU | |
| RNACS5738 | TRAC4_ | mG*mA*mG*rArArUrCrArArArArUrCrGrGrUrGrArArUrGrUrUrUrUrA | 16137 |
| | P9R7_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrCrUrGrUrCrUrCrArCrCrGr | |
| | | A*mU*mU*mU | |
| RNACS5739 | | mG*mA*mG*rArArUrCrArArArArUrCrGrGrUrGrArArUrGrUrUrUrUrA | 16138 |
| | | rGrAmGmCmUmAmGmAmAmAmUmAmGmCrArArGrUrUrArArArArUrArArGr | |
| | | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrUrCrUrGrUrCrUrCrArCrCr | |
| | | G*mA*mU*mU | |
| RNACS5740 | TRAC4_ | mG*mA*mG*rArArUrCrArArArArUrCrGrGrUrGrArArUrGrUrUrUrUrA | 16139 |
| | P13R9_del7_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrArGrUrCrUrGrUrCrUrCrArCr | |
| | | CrGrArUrUrUrU*mG*mA*mU | |
| RNACS5741 | TRAC4_ | mG*mA*mG*rArArUrCrArArArArUrCrGrGrUrGrArArUrGrUrUrUrUrA | 16140 |
| | P12R9_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArGrUrCrUrGrUrCrUrCrArCr | |
| | | CrGrArUrUrU*mU*mG*mA | |
| RNACS5742 | TRAC4_ | mG*mA*mG*rArArUrCrArArArArUrCrGrGrUrGrArArUrGrUrUrUrUrA | 16141 |
| | P11R9_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArGrUrCrUrGrUrCrUrCrArCr | |
| | | CrGrArUrU*mU*mU*mG | |
| RNACS5743 | TRAC4_ | mG*mA*mG*rArArUrCrArArArArUrCrGrGrUrGrArArUrGrUrUrUrUrA | 16142 |
| | P10R9_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArGrUrCrUrGrUrCrUrCrArCr | |
| | | CrGrArU*mU*mU*mU | |
| RNACS5744 | TRAC4_ | mG*mA*mG*rArArUrCrArArArArUrCrGrGrUrGrArArUrGrUrUrUrUrA | 16143 |
| | P9R9_del7_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrArGrUrCrUrGrUrCrUrCrArCr | |
| | | CrGrA*mU*mU*mU | |
| RNACS5745 | TRAC4_ | mG*mA*mG*rArArUrCrArArArArUrCrGrGrUrGrArArUrGrUrUrUrUrA | 16144 |
| | P8R9_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArGrUrCrUrGrUrCrUrCrArCr | |
| | | CrG*mA*mU*mU | |
| RNACS5746 | TRAC4_ | mG*mA*mG*rArArUrCrArArArArUrCrGrGrUrGrArArUrGrUrUrUrUrA | 16145 |
| | P13R11_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArArGrUrCrUrGrUrCrUrCr | |
| | | ArCrCrGrArUrUrUrU*mG*mA*mU | |
| RNACS5747 | TRAC4_ | mG*mA*mG*rArArUrCrArArArArUrCrGrGrUrGrArArUrGrUrUrUrUrA | 16146 |
| | P12R11_del7_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrArArGrUrCrUrGrUrCrUrCr | |
| | | ArCrCrGrArUrUrU*mU*mG*mA | |
| RNACS5748 | TRAC4_ | mG*mA*mG*rArArUrCrArArArArUrCrGrGrUrGrArArUrGrUrUrUrUrA | 16147 |
| | P11R11_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArArGrUrCrUrGrUrCrUrCr | |
| | | ArCrCrGrArUrU*mU*mU*mG | |
| RNACS5749 | TRAC4_ | mG*mA*mG*rArArUrCrArArArArUrCrGrGrUrGrArArUrGrUrUrUrUrA | 16148 |
| | P10R11_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArArGrUrCrUrGrUrCrUrCr | |
| | | ArCrCrGrArU*mU*mU*mU | |
| RNACS5750 | TRAC4_ | mG*mA*mG*rArArUrCrArArArArUrCrGrGrUrGrArArUrGrUrUrUrUrA | 16149 |
| | P9R11_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArArGrUrCrUrGrUrCrUrCr | |
| | | ArCrCrGrA*mU*mU*mU | |
| RNACS5751 | TRAC4_ | mG*mA*mG*rArArUrCrArArArArUrCrGrGrUrGrArArUrGrUrUrUrUrA | 16150 |
| | P8R11_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArArGrUrCrUrGrUrCrUrCr | |
| | | ArCrCrG*mA*mU*mU | |
| RNACS5752 | TRAC4_ | mG*mA*mG*rArArUrCrArArArArUrCrGrGrUrGrArArUrGrUrUrUrUrA | 16151 |
| | P13R13_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArCrArArGrUrCrUrGrUrCr | |
| | | UrCrArCrCrGrArUrUrUrU*mG*mA*mU | |
| RNACS5753 | TRAC4_ | mG*mA*mG*rArArUrCrArArArArUrCrGrGrUrGrArArUrGrUrUrUrUrA | 16152 |
| | P12R13_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArCrArArGrUrCrUrGrUrCr | |
| | | UrCrArCrCrGrArUrUrU*mU*mG*mA | |
| RNACS5754 | TRAC4_ | mG*mA*mG*rArArUrCrArArArArUrCrGrGrUrGrArArUrGrUrUrUrUrA | 16153 |
| | P11R13_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArCrArArGrUrCrUrGrUrCr | |
| | | UrCrArCrCrGrArUrU*mU*mU*mG | |
| RNACS5755 | TRAC4_ | mG*mA*mG*rArArUrCrArArArArUrCrGrGrUrGrArArUrGrUrUrUrUrA | 16154 |
| | P10R13_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArCrArArGrUrCrUrGrUrCr | |
| | | UrCrArCrCrGrArU*mU*mU*mU | |
| RNACS5756 | TRAC4_ | mG*mA*mG*rArArUrCrArArArArUrCrGrGrUrGrArArUrGrUrUrUrUrA | 16155 |
| | P9R13_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArCrArArGrUrCrUrGrUrCr | |
| | | UrCrArCrCrGrA*mU*mU*mU | |
| RNACS5757 | TRAC4_ | mUmAmGmCrArArGrUrUrArArArArUrArArGrGrCrUrArGrUrCrCrGrUr | 16156 |
| | P8R13_del7_ | UrArUrCrAmAmCmUmUmGmAmAmAmAmAmGmUmGmGmCmAmCmCmGmAmGmUmC | |
| | nick | mGmGmUmGmCrGrArCrArArGrUrCrUrGrUrCrUrCrArCrCrG*mA*mU* | |
| | | mUmG*mA*mG*rArArUrCrArArArArUrCrGrGrUrGrArArUrGrUrUrUrU | |
| | | rArGrAmGmCmUmAmGmAmAmA | |
| RNACS5758 | TRAC4_ | mG*mA*mG*rArArUrCrArArArArUrCrGrGrUrGrArArUrGrUrUrUrUrA | 16157 |
| | P13R15_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrUrGrArCrArArGrUrCrUrGr | |
| | | UrCrUrCrArCrCrGrArUrUrUrU*mG*mA*mU | |
| RNACS5759 | TRAC4_ | mG*mA*mG*rArArUrCrArArArArUrCrGrGrUrGrArArUrGrUrUrUrUrA | 16158 |
| | P12R15_del7_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrUrGrArCrArArGrUrCrUrGr | |
| | | UrCrUrCrArCrCrGrArUrUrU*mU*mG*mA | |
| RNACS5760 | TRAC4_ | mG*mA*mG*rArArUrCrArArArArUrCrGrGrUrGrArArUrGrUrUrUrUrA | 16159 |
| | P11R15_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrUrGrArCrArArGrUrCrUrGr | |
| | | UrCrUrCrArCrCrGrArUrU*mU*mU*mG | |
| RNACS5761 | TRAC4_ | mG*mA*mG*rArArUrCrArArArArUrCrGrGrUrGrArArUrGrUrUrUrUrA | 16160 |
| | P10R15_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrUrGrArCrArArGrUrCrUrGr | |
| | | UrCrUrCrArCrCrGrArU*mU*mU*mU | |
| RNACS5762 | TRAC4_ | mG*mA*mG*rArArUrCrArArArArUrCrGrGrUrGrArArUrGrUrUrUrUrA | 16161 |
| | P9R15_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrUrGrArCrArArGrUrCrUrGr | |
| | | UrCrUrCrArCrCrGrA*mU*mU*mU | |
| RNACS5763 | TRAC4_ | mG*mA*mG*rArArUrCrArArArArUrCrGrGrUrGrArArUrGrUrUrUrUrA | 16162 |
| | P8R15_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrUrGrArCrArArGrUrCrUrGr | |
| | | UrCrUrCrArCrCrG*mA*mU*mU | |
| RNACS5764 | TRAC4_ | mG*mA*mG*rArArUrCrArArArArUrCrGrGrUrGrArArUrGrUrUrUrUrA | 16163 |
| | P13R17_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArGrUrGrArCrArArGrUrCr | |
| | | UrGrUrCrUrCrArCrCrGrArUrUrUrU*mG*mA*mU | |
| RNACS5765 | TRAC4_ | mG*mA*mG*rArArUrCrArArArArUrCrGrGrUrGrArArUrGrUrUrUrUrA | 16164 |
| | P12R17_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArGrUrGrArCrArArGrUrCr | |
| | | UrGrUrCrUrCrArCrCrGrArUrUrU*mU*mG*mA | |
| RNACS5766 | TRAC4_ | mG*mA*mG*rArArUrCrArArArArUrCrGrGrUrGrArArUrGrUrUrUrUrA | 16165 |
| | P11R17_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArGrUrGrArCrArArGrUrCr | |
| | | UrGrUrCrUrCrArCrCrGrArUrU*mU*mU*mG | |
| RNACS5767 | TRAC4_ | mG*mA*mG*rArArUrCrArArArArUrCrGrGrUrGrArArUrGrUrUrUrUrA | 16166 |
| | P10R17_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArGrUrGrArCrArArGrUrCr | |
| | | UrGrUrCrUrCrArCrCrGrArU*mU*mU*mU | |
| RNACS5768 | TRAC4_ | mG*mA*mG*rArArUrCrArArArArUrCrGrGrUrGrArArUrGrUrUrUrUrA | 16167 |
| | P9R17_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArGrUrGrArCrArArGrUrCr | |
| | | UrGrUrCrUrCrArCrCrGrA*mU*mU*mU | |
| RNACS5769 | TRAC4_ | mG*mA*mG*rArArUrCrArArArArUrCrGrGrUrGrArArUrGrUrUrUrUrA | 16168 |
| | P8R17_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArGrUrGrArCrArArGrUrCr | |
| | | UrGrUrCrUrCrArCrCrG*mA*mU*mU | |
| RNACS5770 | TRAC4_ | mG*mA*mG*rArArUrCrArArArArUrCrGrGrUrGrArArUrGrUrUrUrUrA | 16169 |
| | P13R13_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrCrCrUrUrArGrUrGrArUr | |
| | | UrCrArCrCrGrArUrUrUrU*mG*mA*mU | |
| RNACS5771 | TRAC4_ | mG*mA*mG*rArArUrCrArArArArUrCrGrGrUrGrArArUrGrUrUrUrUrA | 16170 |
| | P12R13_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrCrCrUrUrArGrUrGrArUr | |
| | | UrCrArCrCrGrArUrUrU*mU*mG*mA | |
| RNACS5772 | TRAC4_ | mG*mA*mG*rArArUrCrArArArArUrCrGrGrUrGrArArUrGrUrUrUrUrA | 16171 |
| | P11R13_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrCrCrUrUrArGrUrGrArUr | |
| | | UrCrArCrCrGrArUrU*mU*mU*mG | |
| RNACS5773 | TRAC4_ | mG*mA*mG*rArArUrCrArArArArUrCrGrGrUrGrArArUrGrUrUrUrUrA | 16172 |
| | P10R13_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrCrCrUrUrArGrUrGrArUr | |
| | | UrCrArCrCrGrArU*mU*mU*mU | |
| RNACS5774 | TRAC4_ | mG*mA*mG*rArArUrCrArArArArUrCrGrGrUrGrArArUrGrUrUrUrUrA | 16173 |
| | P9R13_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrCrCrUrUrArGrUrGrArUr | |
| | | UrCrArCrCrGrA*mU*mU*mU | |
| RNACS5775 | TRAC4_ | mUmAmGmCrArArGrUrUrArArArArUrArArGrGrCrUrArGrUrCrCrGrUr | 16174 |
| | P8R13_ | UrArUrCrAmAmCmUmUmGmAmAmAmAmAmGmUmGmGmCmAmCmCmGmAmGmUmC | |
| | ins5- | mGmGmUmGmCrUrGrCrCrUrUrArGrUrGrArUrUrCrArCrCrG*mA*mU* | |
| | PAM | mUmG*mA*mG*rArArUrCrArArArArUrCrGrGrUrGrArArUrGrUrUrUrU | |
| | | rArGrAmGmCmUmAmGmAmAmA | |
| RNACS5776 | TRAC4_ | mG*mA*mG*rArArUrCrArArArArUrCrGrGrUrGrArArUrGrUrUrUrUrA | 16175 |
| | P13R15_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrUrGrCrCrUrUrArGrUrGr | |
| | | ArUrUrCrArCrCrGrArUrUrUrU*mG*mA*mU | |
| RNACS5777 | TRAC4_ | mG*mA*mG*rArArUrCrArArArArUrCrGrGrUrGrArArUrGrUrUrUrUrA | 16176 |
| | P12R15_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrUrGrCrCrUrUrArGrUrGr | |
| | | ArUrUrCrArCrCrGrArUrUrU*mUmG*mA | |
| RNACS5778 | TRAC4_ | mG*mA*mG*rArArUrCrArArArArUrCrGrGrUrGrArArUrGrUrUrUrUrA | 16177 |
| | P11R15_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrUrGrCrCrUrUrArGrUrGr | |
| | | ArUrUrCrArCrCrGrArUrU*mU*mU*mG | |
| RNACS5779 | TRAC4_ | mG*mA*mG*rArArUrCrArArArArUrCrGrGrUrGrArArUrGrUrUrUrUrA | 16178 |
| | P10R15_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrUrGrCrCrUrUrArGrUrGr | |
| | | ArUrUrCrArCrCrGrArU*mU*mU*mU | |
| RNACS5780 | TRAC4_ | mG*mA*mG*rArArUrCrArArArArUrCrGrGrUrGrArArUrGrUrUrUrUrA | 16179 |
| | P9R15_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrUrGrCrCrUrUrArGrUrGr | |
| | | ArUrUrCrArCrCrGrA*mU*mU*mU | |
| RNACS5781 | TRAC4_ | mG*mA*mG*rArArUrCrArArArArUrCrGrGrUrGrArArUrGrUrUrUrUrA | 16180 |
| | P8R15_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrUrGrCrCrUrUrArGrUrGr | |
| | | ArUrUrCrArCrCrG*mA*mU*mU | |
| RNACS5782 | TRAC4_ | mG*mA*mG*rArArUrCrArArArArUrCrGrGrUrGrArArUrGrUrUrUrUrA | 16181 |
| | P13R17_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrUrCrUrGrCrCrUrUrArGr | |
| | | UrGrArUrUrCrArCrCrGrArUrUrUrU*mG*mA*mU | |
| RNACS5783 | TRAC4_ | mG*mA*mG*rArArUrCrArArArArUrCrGrGrUrGrArArUrGrUrUrUrUrA | 16182 |
| | P12R17_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrUrCrUrGrCrCrUrUrArGr | |
| | | UrGrArUrUrCrArCrCrGrArUrUrU*mU*mG*mA | |
| RNACS5784 | TRAC4_ | mG*mA*mG*rArArUrCrArArArArUrCrGrGrUrGrArArUrGrUrUrUrUrA | 16183 |
| | P12R19_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrUrCrUrGrCrCrUrUrAr | |
| | | GrUrGrArUrUrCrArCrCrGrArUrU*mU*mU*mG | |
| RNACS5785 | TRAC4_ | mG*mA*mG*rArArUrCrArArArArUrCrGrGrUrGrArArUrGrUrUrUrUrA | 16184 |
| | P10R17_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrUrCrUrGrCrCrUrUrArGr | |
| | | UrGrArUrUrCrArCrCrGrArU*mU*mU*mU | |
| RNACS5786 | TRAC4_ | mG*mA*mG*rArArUrCrArArArArUrCrGrGrUrGrArArUrGrUrUrUrUrA | 16185 |
| | P9R17_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrUrCrUrGrCrCrUrUrArGr | |
| | | UrGrArUrUrCrArCrCrGrA*mU*mU*mU | |
| RNACS5787 | TRAC4_ | mG*mA*mG*rArArUrCrArArArArUrCrGrGrUrGrArArUrGrUrUrUrUrA | 16186 |
| | P8R17_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrUrCrUrGrCrCrUrUrArGr | |
| | | UrGrArUrUrCrArCrCrG*mA*mU*mU | |
| RNACS5788 | TRAC4_ | mG*mA*mG*rArArUrCrArArArArUrCrGrGrUrGrArArUrGrUrUrUrUrA | 16187 |
| | P13R19_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrUrGrUrCrUrGrCrCrUrUr | |
| | | ArGrUrGrArUrUrCrArCrCrGrArUrUrUrU*mG*mA*mU | |
| RNACS5789 | TRAC4_ | mG*mA*mG*rArArUrCrArArArArUrCrGrGrUrGrArArUrGrUrUrUrUrA | 16188 |
| | P12R19_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrUrGrUrCrUrGrCrCrUrUr | |
| | | ArGrUrGrArUrUrCrArCrCrGrArUrUrU*mU*mG*mA | |
| RNACS5790 | TRAC4_ | mG*mA*mG*rArArUrCrArArArArUrCrGrGrUrGrArArUrGrUrUrUrUrA | 16189 |
| | P11R19_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrUrGrUrCrUrGrCrCrUrUr | |
| | | ArGrUrGrArUrUrCrArCrCrGrArUrU*mU*mU*mG | |
| RNACS5791 | TRAC4_ | mG*mA*mG*rArArUrCrArArArArUrCrGrGrUrGrArArUrGrUrUrUrUrA | 16190 |
| | P10R19_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrUrGrUrCrUrGrCrCrUrUr | |
| | | ArGrUrGrArUrUrCrArCrCrGrArU*mU*mU*mU | |
| RNACS5792 | TRAC4_ | mG*mA*mG*rArArUrCrArArArArUrCrGrGrUrGrArArUrGrUrUrUrUrA | 16191 |
| | P9R19_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrUrGrUrCrUrGrCrCrUrUr | |
| | | ArGrUrGrArUrUrCrArCrCrGrA*mU*mU*mU | |
| RNACS5793 | TRAC4_ | mG*mA*mG*rArArUrCrArArArArUrCrGrGrUrGrArArUrGrUrUrUrUrA | 16192 |
| | P8R19_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrUrGrUrCrUrGrCrCrUr | |
| | | UrArGrUrGrArUrUrCrArCrCrG*mA*mU*mU | |
| RNACS5794 | TRAC4_ | mG*mA*mG*rArArUrCrArArArArUrCrGrGrUrGrArArUrGrUrUrUrUrA | 16193 |
| | P13R21_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrUrCrUrGrUrCrUrGrCrCr | |
| | | UrUrArGrUrGrArUrUrCrArCrCrGrArUrUrUrU*mG*mA*mU | |
| RNACS5795 | TRAC4_ | mG*mA*mG*rArArUrCrArArArArUrCrGrGrUrGrArArUrGrUrUrUrUrA | 16194 |
| | P12R21_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrUrCrUrGrUrCrUrGrCrCr | |
| | | UrUrArGrUrGrArUrUrCrArCrCrGrArUrUrU*mU*mG*mA | |
| RNACS5796 | TRAC4_ | mG*mA*mG*rArArUrCrArArArArUrCrGrGrUrGrArArUrGrUrUrUrUrA | 16195 |
| | P11R21_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrUrCrUrGrUrCrUrGrCrCr | |
| | | UrUrArGrUrGrArUrUrCrArCrCrGrArUrU*mU*mU*mG | |
| RNACS5797 | TRAC4_ | mG*mA*mG*rArArUrCrArArArArUrCrGrGrUrGrArArUrGrUrUrUrUrA | 16196 |
| | P10R21_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrUrCrUrGrUrCrUrGrCrCr | |
| | | UrUrArGrUrGrArUrUrCrArCrCrGrArU*mU*mU*mU | |
| RNACS5798 | TRAC4_ | mG*mA*mG*rArArUrCrArArArArUrCrGrGrUrGrArArUrGrUrUrUrUrA | 16197 |
| | P9R21_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrUrCrUrGrUrCrUrGrCrCr | |
| | | UrUrArGrUrGrArUrUrCrArCrCrGrA*mU*mU*mU | |
| RNACS5799 | TRAC4_ | mG*mA*mG*rArArUrCrArArArArUrCrGrGrUrGrArArUrGrUrUrUrUrA | 16198 |
| | P8R21_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrUrCrUrGrUrCrUrGrCrCr | |
| | | UrUrArGrUrGrArUrUrCrArCrCrG*mA*mU*mU | |
| RNACS5800 | TRAC4_ | mG*mA*mG*rArArUrCrArArArArUrCrGrGrUrGrArArUrGrUrUrUrUrA | 16199 |
| | P13R23_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArArGrUrCrUrGrUrCrUrGr | |
| | | CrCrUrUrArGrUrGrArUrUrCrArCrCrGrArUrUrUrU*mG*mA*mU | |
| RNACS5801 | TRAC4_ | mG*mA*mG*rArArUrCrArArArArUrCrGrGrUrGrArArUrGrUrUrUrUrA | 16200 |
| | P12R23_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArArGrUrCrUrGrUrCrUrGr | |
| | | CrCrUrUrArGrUrGrArUrUrCrArCrCrGrArUrUrU*mU*mG*mA | |
| RNACS5802 | TRAC4_ | mG*mA*mG*rArArUrCrArArArArUrCrGrGrUrGrArArUrGrUrUrUrUrAr | 16201 |
| | P11R23_ | GrAmGmCmUmAmGmAmAmAmUmAmGmCrArArGrUrUrArArArArUrArArGrGr | |
| | ins5- | CrUrArGrUrCrCrGrUrUrArUrCrAmAmCmUmUmGmAmAmAmAmAmGmUmGmGm | |
| | PAM | CmAmCmCmGmAmGmUmCmGmGmUmGmCrCrArArGrUrCrUrGrUrCrUr | |
| | | GrCrCrUrUrArGrUrGrArUrUrCrArCrCrGrArUrUmU*mU*mG | |
| RNACS5803 | TRAC4_ | mG*mA*mG*rArArUrCrArArArArUrCrGrGrUrGrArArUrGrUrUrUrUrA | 16202 |
| | P10R23_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArArGrUrCrUrGrUrCrUrGr | |
| | | CrCrUrUrArGrUrGrArUrUrCrArCrCrGrArU*mU*mU*mU | |
| RNACS5804 | TRAC4_ | mG*mA*mG*rArArUrCrArArArArUrCrGrGrUrGrArArUrGrUrUrUrUrA | 16203 |
| | P9R23_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArArGrUrCrUrGrUrCrUrGr | |
| | | CrCrUrUrArGrUrGrArUrUrCrArCrCrGrA*mU*mU*mU | |
| RNACS5805 | TRAC4_ | mG*mA*mG*rArArUrCrArArArArUrCrGrGrUrGrArArUrGrUrUrUrUrA | 16204 |
| | P8R23_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArArGrUrCrUrGrUrCrUrGr | |
| | | CrCrUrUrArGrUrGrArUrUrCrArCrCrG*mA*mU*mU | |
| RNACS5806 | TRAC4_ | mG*mA*mG*rArArUrCrArArArArUrCrGrGrUrGrArArUrGrUrUrUrUrA | 16205 |
| | P13R25_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrArCrArArGrUrCrUrGrUrCr | |
| | | UrGrCrCrUrUrArGrUrGrArUrUrCrArCrCrGrArUrUrUrUmG*mA*mU | |
| RNACS5807 | TRAC4_ | mG*mA*mG*rArArUrCrArArArArUrCrGrGrUrGrArArUrGrUrUrUrUrA | 16206 |
| | P12R25_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrArCrArArGrUrCrUrGrUrCr | |
| | | UrGrCrCrUrUrArGrUrGrArUrUrCrArCrCrGrArUrUrU*mU*mG*mA | |
| RNACS5808 | TRAC4_ | mG*mA*mG*rArArUrCrArArArArUrCrGrGrUrGrArArUrGrUrUrUrUrA | 16207 |
| | P11R25_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrArCrArArGrUrCrUrGrUrCr | |
| | | UrGrCrCrUrUrArGrUrGrArUrUrCrArCrCrGrArUrU*mU*mU*mG | |
| RNACS5809 | TRAC4_ | mG*mA*mG*rArArUrCrArArArArUrCrGrGrUrGrArArUrGrUrUrUrUrA | 16208 |
| | P10R25_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrArCrArArGrUrCrUrGrUrCr | |
| | | UrGrCrCrUrUrArGrUrGrArUrUrCrArCrCrGrArU*mU*mU*mU | |
| RNACS5810 | TRAC4_ | mG*mA*mG*rArArUrCrArArArArUrCrGrGrUrGrArArUrGrUrUrUrUrA | 16209 |
| | P9R25_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrArCrArArGrUrCrUrGrUrCr | |
| | | UrGrCrCrUrUrArGrUrGrArUrUrCrArCrCrGrA*mU*mU*mU | |
| RNACS5810 | TRAC4_ | mG*mA*mG*rArArUrCrArArArArUrCrGrGrUrGrArArUrGrUrUrUrUrA | 16210 |
| | P8R25_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrArCrArArGrUrCrUrGrUr | |
| | | CrUrGrCrCrUrUrArGrUrGrArUrUrCrArCrCrG*mA*mU*mU | |
| RNACS5812 | TRAC4_ | mG*mA*mG*rArArUrCrArArArArUrCrGrGrUrGrArArUrGrUrUrUrUrA | 16211 |
| | P13R15_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrCrCrUrUrArGrUrUrGrAr | |
| | | ArUrUrCrArCrCrGrArUrUrUrU*mG*mA*mU | |
| RNACS5813 | TRAC4_ | mG*mA*mG*rArArUrCrArArArArUrCrGrGrUrGrArArUrGrUrUrUrUrA | 16212 |
| | P12R15_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrCrCrUrUrArGrUrUrGrAr | |
| | | ArUrUrCrArCrCrGrArUrUrU*mU*mG*mA | |
| RNACS5814 | TRAC4_ | mG*mA*mG*rArArUrCrArArArArUrCrGrGrUrGrArArUrGrUrUrUrUrA | 16213 |
| | P11R15_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrCrCrUrUrArGrUrUrGrAr | |
| | | ArUrUrCrArCrCrGrArUrU*mU*mU*mG | |
| RNACS5815 | TRAC4_ | mG*mA*mG*rArArUrCrArArArArUrCrGrGrUrGrArArUrGrUrUrUrUrA | 16214 |
| | P10R15_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrCrCrUrUrArGrUrUrGrAr | |
| | | ArUrUrCrArCrCrGrArU*mU*mU*mU | |
| RNACS5816 | TRAC4_ | mG*mA*mG*rArArUrCrArArArArUrCrGrGrUrGrArArUrGrUrUrUrUrA | 16215 |
| | P9R15_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrCrCrUrUrArGrUrUrGrAr | |
| | | ArUrUrCrArCrCrGrA*mU*mU*mU | |
| RNACS5817 | TRAC4_ | mG*mA*mG*rArArUrCrArArArArUrCrGrGrUrGrArArUrGrUrUrUrUrA | 16216 |
| | P8R15_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrCrCrUrUrArGrUrUrGrAr | |
| | | ArUrUrCrArCrCrG*mA*mU*mU | |
| RNACS5818 | TRAC4_ | mG*mA*mG*rArArUrCrArArArArUrCrGrGrUrGrArArUrGrUrUrUrUrA | 16217 |
| | P13R17_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrUrGrCrCrUrUrArGrUrUr | |
| | | GrArArUrUrCrArCrCrGrArUrUrUrU*mG*mA*mU | |
| RNACS5819 | TRAC4_ | mG*mA*mG*rArArUrCrArArArArUrCrGrGrUrGrArArUrGrUrUrUrUrA | 16218 |
| | P12R17_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrUrGrCrCrUrUrArGrUrUr | |
| | | GrArArUrUrCrArCrCrGrArUrUrU*mU*mG*mA | |
| RNACS5820 | TRAC4_ | mG*mA*mG*rArArUrCrArArArArUrCrGrGrUrGrArArUrGrUrUrUrUrA | 16219 |
| | P11R17_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrUrGrCrCrUrUrArGrUr | |
| | | UrGrArArUrUrCrArCrCrGrArUrU*mU*mU*mG | |
| RNACS5821 | TRAC4_ | mG*mA*mG*rArArUrCrArArArArUrCrGrGrUrGrArArUrGrUrUrUrUrA | 16220 |
| | P10R17_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrUrGrCrCrUrUrArGrUrUr | |
| | | GrArArUrUrCrArCrCrGrArU*mU*mU*mU | |
| RNACS5822 | TRAC4_ | mG*mA*mG*rArArUrCrArArArArUrCrGrGrUrGrArArUrGrUrUrUrUrA | 16221 |
| | P9R17_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrUrGrCrCrUrUrArGrUrUr | |
| | | GrArArUrUrCrArCrCrGrA*mU*mU*mU | |
| RNACS5823 | TRAC4_ | mG*mA*mG*rArArUrCrArArArArUrCrGrGrUrGrArArUrGrUrUrUrUrA | 16222 |
| | P8R17_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrUrGrCrCrUrUrArGrUrUr | |
| | | GrArArUrUrCrArCrCrG*mA*mU*mU | |
| RNACS5824 | TRAC4_ | mG*mA*mG*rArArUrCrArArArArUrCrGrGrUrGrArArUrGrUrUrUrUrA | 16223 |
| | P13R19_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrUrCrUrGrCrCrUrUrArGr | |
| | | UrUrGrArArUrUrCrArCrCrGrArUrUrUrU*mG*mA*mU | |
| RNACS5825 | TRAC4_ | mG*mA*mG*rArArUrCrArArArArUrCrGrGrUrGrArArUrGrUrUrUrUrA | 16224 |
| | P12R19_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrUrCrUrGrCrCrUrUrArGr | |
| | | UrUrGrArArUrUrCrArCrCrGrArUrUrU*mU*mG*mA | |
| RNACS5826 | TRAC4_ | mG*mA*mG*rArArUrCrArArArArUrCrGrGrUrGrArArUrGrUrUrUrUrA | 16225 |
| | P11R19_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrUrCrUrGrCrCrUrUrArGr | |
| | | UrUrGrArArUrUrCrArCrCrGrArUrU*mU*mU*mG | |
| RNACS5827 | TRAC4_ | mG*mA*mG*rArArUrCrArArArArUrCrGrGrUrGrArArUrGrUrUrUrUrA | 16226 |
| | P10R19_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrUrCrUrGrCrCrUrUrArGr | |
| | | UrUrGrArArUrUrCrArCrCrGrArU*mU*mU*mU | |
| RNACS5828 | TRAC4_ | mG*mA*mG*rArArUrCrArArArArUrCrGrGrUrGrArArUrGrUrUrUrUrA | 16227 |
| | P9R19_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrUrCrUrGrCrCrUrUrArGr | |
| | | UrUrGrArArUrUrCrArCrCrGrA*mU*mU*mU | |
| RNACS5829 | | mG*mA*mG*rArArUrCrArArArArUrCrGrGrUrGrArArUrGrUrUrUrUrA | 16228 |
| | | rGrAmGmCmUmAmGmAmAmAmUmAmGmCrArArGrUrUrArArArArUrArArGr | |
| | | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrUrGrUrCrUrGrCrCrUrUrAr | |
| | | GrUrUrGrArArUrUrCrArCrCrG*mA*mU*mU | |
| RNACS5830 | TRAC4_ | mG*mA*mG*rArArUrCrArArArArUrCrGrGrUrGrArArUrGrUrUrUrUrA | 16229 |
| | P13R21_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrUrGrUrCrUrGrCrCrUrUr | |
| | | ArGrUrUrGrArArUrUrCrArCrCrGrArUrUrUrU*mG*mA*mU | |
| RNACS5831 | TRAC4_ | mG*mA*mG*rArArUrCrArArArArUrCrGrGrUrGrArArUrGrUrUrUrUrA | 16230 |
| | P12R21_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrUrGrUrCrUrGrCrCrUrUr | |
| | | ArGrUrUrGrArArUrUrCrArCrCrGrArUrUrU*mU*mG*mA | |
| RNACS5832 | TRAC4_ | mG*mA*mG*rArArUrCrArArArArUrCrGrGrUrGrArArUrGrUrUrUrUrA | 16231 |
| | P11R21_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrUrGrUrCrUrGrCrCrUrUr | |
| | | ArGrUrUrGrArArUrUrCrArCrCrGrArUrU*mU*mU*mG | |
| RNACS5833 | TRAC4_ | mG*mA*mG*rArArUrCrArArArArUrCrGrGrUrGrArArUrGrUrUrUrUrA | 16232 |
| | P10R21_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrUrGrUrCrUrGrCrCrUrUr | |
| | | ArGrUrUrGrArArUrUrCrArCrCrGrArU*mU*mU*mU | |
| RNACS5834 | TRAC4_ | mG*mA*mG*rArArUrCrArArArArUrCrGrGrUrGrArArUrGrUrUrUrUrA | 16233 |
| | P9R21_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrUrGrUrCrUrGrCrCrUrUr | |
| | | ArGrUrUrGrArArUrUrCrArCrCrGrA*mU*mU*mU | |
| RNACS5835 | TRAC4_ | mG*mA*mG*rArArUrCrArArArArUrCrGrGrUrGrArArUrGrUrUrUrUrA | 16234 |
| | P8R21_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrUrGrUrCrUrGrCrCrUrUr | |
| | | ArGrUrUrGrArArUrUrCrArCrCrG*mA*mU*mU | |
| RNACS5836 | TRAC4_ | mG*mA*mG*rArArUrCrArArArArUrCrGrGrUrGrArArUrGrUrUrUrUrA | 16235 |
| | P13R23_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrUrCrUrGrUrCrUrGrCrCr | |
| | | UrUrArGrUrUrGrArArUrUrCrArCrCrGrArUrUrUrU*mG*mA*mU | |
| RNACS5837 | TRAC4_ | mG*mA*mG*rArArUrCrArArArArUrCrGrGrUrGrArArUrGrUrUrUrUrA | 16236 |
| | P12R23_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrUrCrUrGrUrCrUrGrCrCr | |
| | | UrUrArGrUrUrGrArArUrUrCrArCrCrGrArUrUrU*mU*mG*mA | |
| RNACS5838 | TRAC4_ | mG*mA*mG*rArArUrCrArArArArUrCrGrGrUrGrArArUrGrUrUrUrUrA | 16237 |
| | P11R23_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrUrCrUrGrUrCrUrGrCrCr | |
| | | UrUrArGrUrUrGrArArUrUrCrArCrCrGrArUrU*mU*mU*mG | |
| RNACS5839 | TRAC4_ | mG*mA*mG*rArArUrCrArArArArUrCrGrGrUrGrArArUrGrUrUrUrUrA | 16238 |
| | P10R23_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrUrCrUrGrUrCrUrGrCrCr | |
| | | UrUrArGrUrUrGrArArUrUrCrArCrCrGrArU*mU*mU*mU | |
| RNACS5840 | TRAC4_ | mG*mA*mG*rArArUrCrArArArArUrCrGrGrUrGrArArUrGrUrUrUrUrA | 16239 |
| | P9R23_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrUrCrUrGrUrCrUrGrCrCr | |
| | | UrUrArGrUrUrGrArArUrUrCrArCrCrGrA*mU*mU*mU | |
| RNACS5841 | TRAC4_ | mG*mA*mG*rArArUrCrArArArArUrCrGrGrUrGrArArUrGrUrUrUrUrA | 16240 |
| | P8R23_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrUrCrUrGrUrCrUrGrCrCr | |
| | | UrUrArGrUrUrGrArArUrUrCrArCrCrG*mA*mU*mU | |
| RNACS5842 | TRAC4_ | mG*mA*mG*rArArUrCrArArArArUrCrGrGrUrGrArArUrGrUrUrUrUrA | 16241 |
| | P13R25_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArArGrUrCrUrGrUrCrUrGr | |
| | | CrCrUrUrArGrUrUrGrArArUrUrCrArCrCrGrArUrUrUrU*mG*mA*mU | |
| RNACS5843 | TRAC4_ | mG*mA*mG*rArArUrCrArArArArUrCrGrGrUrGrArArUrGrUrUrUrUrA | 16242 |
| | P12R25_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArArGrUrCrUrGrUrCrUrGr | |
| | | CrCrUrUrArGrUrUrGrArArUrUrCrArCrCrGrArUrUrU*mU*mG*mA | |
| RNACS5844 | TRAC4_ | mG*mA*mG*rArArUrCrArArArArUrCrGrGrUrGrArArUrGrUrUrUrUrA | 16243 |
| | P11R25_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArArGrUrCrUrGrUrCrUrGr | |
| | | CrCrUrUrArGrUrUrGrArArUrUrCrArCrCrGrArUrU*mU*mU*mG | |
| RNACS5845 | TRAC4_ | mG*mA*mG*rArArUrCrArArArArUrCrGrGrUrGrArArUrGrUrUrUrUrA | 16244 |
| | P10R25_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArArGrUrCrUrGrUrCrUrGr | |
| | | CrCrUrUrArGrUrUrGrArArUrUrCrArCrCrGrArU*mU*mU*mU | |
| RNACS5846 | TRAC4_ | mG*mA*mG*rArArUrCrArArArArUrCrGrGrUrGrArArUrGrUrUrUrUrA | 16245 |
| | P9R25_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArArGrUrCrUrGrUrCrUrGr | |
| | | CrCrUrUrArGrUrUrGrArArUrUrCrArCrCrGrA*mU*mU*mU | |
| RNACS5847 | TRAC4_ | mG*mA*mG*rArArUrCrArArArArUrCrGrGrUrGrArArUrGrUrUrUrUrA | 16246 |
| | P8R25_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArArGrUrCrUrGrUrCrUr | |
| | | GrCrCrUrUrArGrUrUrGrArArUrUrCrArCrCrG*mA*mU*mU | |
| RNACS5848 | TRAC4_ | mG*mA*mG*rArArUrCrArArArArUrCrGrGrUrGrArArUrGrUrUrUrUrA | 16247 |
| | P13R27_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrArCrArArGrUrCrUrGrUrCr | |
| | | UrGrCrCrUrUrArGrUrUrGrArArUrUrCrArCrCrGrArUrUrUrU*mG*mA | |
| | | *mU | |
| RNACS5849 | TRAC4_ | mG*mA*mG*rArArUrCrArArArArUrCrGrGrUrGrArArUrGrUrUrUrUrA | 16248 |
| | P12R27_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrArCrArArGrUrCrUrGrUrCr | |
| | | UrGrCrCrUrUrArGrUrUrGrArArUrUrCrArCrCrGrArUrUrU*mU*mG*m | |
| | | A | |
| RNACS5850 | TRAC4_ | mG*mA*mG*rArArUrCrArArArArUrCrGrGrUrGrArArUrGrUrUrUrUrA | 16249 |
| | P11R27_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrArCrArArGrUrCrUrGrUrCr | |
| | | UrGrCrCrUrUrArGrUrUrGrArArUrUrCrArCrCrGrArUrU*mU*mU*mG | |
| RNACS5851 | TRAC4_ | mG*mA*mG*rArArUrCrArArArArUrCrGrGrUrGrArArUrGrUrUrUrUrA | 16250 |
| | P10R27_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrArCrArArGrUrCrUrGrUrCr | |
| | | UrGrCrCrUrUrArGrUrUrGrArArUrUrCrArCrCrGrArU*mU*mU*mU | |
| RNACS5852 | TRAC4_ | mG*mA*mG*rArArUrCrArArArArUrCrGrGrUrGrArArUrGrUrUrUrUrA | 16251 |
| | P9R27_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrArCrArArGrUrCrUrGrUrCr | |
| | | UrGrCrCrUrUrArGrUrUrGrArArUrUrCrArCrCrGrA*mU*mU*mU | |
| RNACS5853 | TRAC4_ | mG*mA*mG*rArArUrCrArArArArUrCrGrGrUrGrArArUrGrUrUrUrUrA | 16252 |
| | P8R27_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrArCrArArGrUrCrUrGrUrCr | |
| | | UrGrCrCrUrUrArGrUrUrGrArArUrUrCrArCrCrG*mA*mU*mU | |
| RNACS5854 | TRAC4_ | mG*mA*mG*rArArUrCrArArArArUrCrGrGrUrGrArArUrGrUrUrUrUrA | 16253 |
| | P13R8_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrUrGrUrCrArUrUrCrArCrCr | |
| | | GrArUrUrUrU*mG*mA*mU | |
| RNACS5855 | TRAC4_ | mG*mA*mG*rArArUrCrArArArArUrCrGrGrUrGrArArUrGrUrUrUrUrA | 16254 |
| | P12R8_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrUrGrUrCrArUrUrCrArCrCr | |
| | | GrArUrUrU*mU*mG*mA | |
| RNACS5856 | | mG*mA*mG*rArArUrCrArArArArUrCrGrGrUrGrArArUrGrUrUrUrUrA | 16255 |
| | | rGrAmGmCmUmAmGmAmAmAmUmAmGmCrArArGrUrUrArArArArUrArArGr | |
| | | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrGrUrCrArUrUrCrArCr | |
| | | CrGrArUrUmU*mU*mG | |
| RNACS5857 | TRAC4_ | mG*mA*mG*rArArUrCrArArArArUrCrGrGrUrGrArArUrGrUrUrUrUrA | 16256 |
| | P10R8_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrUrGrUrCrArUrUrCrArCrCr | |
| | | GrArU*mU*mU*mU | |
| RNACS5858 | TRAC4_ | mG*mA*mG*rArArUrCrArArArArUrCrGrGrUrGrArArUrGrUrUrUrUrA | 16257 |
| | P9R8_del5- | rGrAmGmCmUmAmGmAmAmAmUmAmGmCrArArGrUrUrArArArArUrArArGr | |
| | PAM | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrUrGrUrCrArUrUrCrArCrCr | |
| | | GrA*mU*mU*mU | |
| RNACS5859 | TRAC4_ | mG*mA*mG*rArArUrCrArArArArUrCrGrGrUrGrArArUrGrUrUrUrUrA | 16258 |
| | P8R8_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrUrGrUrCrArUrUrCrArCrCr | |
| | | G*mA*mU*mU | |
| RNACS5860 | TRAC4_ | mG*mA*mG*rArArUrCrArArArArUrCrGrGrUrGrArArUrGrUrUrUrUrA | 16259 |
| | P13R10_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrUrCrUrGrUrCrArUrUrCrAr | |
| | | CrCrGrArUrUrUrU*mG*mA*mU | |
| RNACS5861 | TRAC4_ | mG*mA*mG*rArArUrCrArArArArUrCrGrGrUrGrArArUrGrUrUrUrUrA | 16260 |
| | P12R10_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrUrCrUrGrUrCrArUrUrCrAr | |
| | | CrCrGrArUrUrU*mU*mG*mA | |
| RNACS5862 | TRAC4_ | mG*mA*mG*rArArUrCrArArArArUrCrGrGrUrGrArArUrGrUrUrUrUrA | 16261 |
| | P11R10_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrUrCrUrGrUrCrArUrUrCrAr | |
| | | CrCrGrArUrU*mU*mU*mG | |
| RNACS5863 | TRAC4_ | mG*mA*mG*rArArUrCrArArArArUrCrGrGrUrGrArArUrGrUrUrUrUrA | 16262 |
| | P10R10_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrUrCrUrGrUrCrArUrUrCrAr | |
| | | CrCrGrArU*mU*mU*mU | |
| RNACS5864 | TRAC4_ | mG*mA*mG*rArArUrCrArArArArUrCrGrGrUrGrArArUrGrUrUrUrUrA | 16263 |
| | P9R10_del5- | rGrAmGmCmUmAmGmAmAmAmUmAmGmCrArArGrUrUrArArArArUrArArGr | |
| | PAM | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrGrUrCrUrGrUrCrArUrUrCrAr | |
| | | CrCrGrA*mU*mU*mU | |
| RNACS5865 | TRAC4_ | mG*mA*mG*rArArUrCrArArArArUrCrGrGrUrGrArArUrGrUrUrUrUrA | 16264 |
| | P8R10_ | rGrAmGmCmUmAmGmAmAmAmUmAmGmCrArArGrUrUrArArArArUrArArGr | |
| | de15- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrUrCrUrGrUrCrArUrUrCrAr | |
| | | CrCrG*mA*mU*mU | |
| RNACS5866 | TRAC4_ | mG*mA*mG*rArArUrCrArArArArUrCrGrGrUrGrArArUrGrUrUrUrUrA | 16265 |
| | P13R12_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArArGrUrCrUrGrUrCrArUrUr | |
| | | CrArCrCrGrArUrUrUrUmG*mA*mU | |
| RNACS5867 | TRAC4_ | mG*mA*mG*rArArUrCrArArArArUrCrGrGrUrGrArArUrGrUrUrUrUrA | 16266 |
| | P12R12_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArArGrUrCrUrGrUrCrArUrUr | |
| | | CrArCrCrGrArUrUrU*mU*mG*mA | |
| RNACS5868 | TRAC4_ | mG*mA*mG*rArArUrCrArArArArUrCrGrGrUrGrArArUrGrUrUrUrUrA | 16267 |
| | P11R12_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArArGrUrCrUrGrUrCrArUrUr | |
| | | CrArCrCrGrArUrU*mU*mU*mG | |
| RNACS5869 | TRAC4_ | mG*mA*mG*rArArUrCrArArArArUrCrGrGrUrGrArArUrGrUrUrUrUrA | 16268 |
| | P10R12_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArArGrUrCrUrGrUrCrArUrUr | |
| | | CrArCrCrGrArU*mU*mU*mU | |
| RNACS5870 | TRAC4_ | mG*mA*mG*rArArUrCrArArArArUrCrGrGrUrGrArArUrGrUrUrUrUrA | 16269 |
| | POR12_del5- | rGrAmGmCmUmAmGmAmAmAmUmAmGmCrArArGrUrUrArArArArUrArArGr | |
| | PAM | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrArArGrUrCrUrGrUrCrArUrUr | |
| | | CrArCrCrGrA*mU*mU*mU | |
| RNACS5871 | TRAC4_ | mG*mA*mG*rArArUrCrArArArArUrCrGrGrUrGrArArUrGrUrUrUrUrA | 16270 |
| | P8R12_ | rGrAmGmCmUmAmGmAmAmAmUmAmGmCrArArGrUrUrArArArArUrArArGr | |
| | de15- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArArGrUrCrUrGrUrCrArUrUr | |
| | | CrArCrCrG*mA*mU*mU | |
| RNACS5872 | TRAC4_ | mG*mA*mG*rArArUrCrArArArArUrCrGrGrUrGrArArUrGrUrUrUrUrA | 16271 |
| | P13R14_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArCrArArGrUrCrUrGrUrCrAr | |
| | | UrUrCrArCrCrGrArUrUrUrU*mG*mA*mU | |
| RNACS5873 | TRAC4_ | mG*mA*mG*rArArUrCrArArArArUrCrGrGrUrGrArArUrGrUrUrUrUrA | 16272 |
| | P12R14_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArCrArArGrUrCrUrGrUrCrAr | |
| | | UrUrCrArCrCrGrArUrUrU*mU*mG*mA | |
| RNACS5874 | TRAC4_ | mG*mA*mG*rArArUrCrArArArArUrCrGrGrUrGrArArUrGrUrUrUrUrA | 16273 |
| | P11R14_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArCrArArGrUrCrUrGrUrCr | |
| | | ArUrUrCrArCrCrGrArUrU*mU*mU*mG | |
| RNACS5875 | TRAC4_ | mG*mA*mG*rArArUrCrArArArArUrCrGrGrUrGrArArUrGrUrUrUrUrA | 16274 |
| | P10R14_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArCrArArGrUrCrUrGrUrCrAr | |
| | | UrUrCrArCrCrGrArU*mU*mU*mU | |
| RNACS5876 | TRAC4_ | mG*mA*mG*rArArUrCrArArArArUrCrGrGrUrGrArArUrGrUrUrUrUrA | 16275 |
| | P9R14_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArCrArArGrUrCrUrGrUrCrAr | |
| | | UrUrCrArCrCrGrA*mU*mU*mU | |
| RNACS5877 | TRAC4_ | mG*mA*mG*rArArUrCrArArArArUrCrGrGrUrGrArArUrGrUrUrUrUrA | 16276 |
| | P8R14_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArCrArArGrUrCrUrGrUrCrAr | |
| | | UrUrCrArCrCrG*mA*mU*mU | |
| RNACS5878 | TRAC4_ | mG*mA*mG*rArArUrCrArArArArUrCrGrGrUrGrArArUrGrUrUrUrUrA | 16277 |
| | P13R16_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrArCrArArGrUrCrUrGrUr | |
| | | CrArUrUrCrArCrCrGrArUrUrUrU*mG*mA*mU | |
| RNACS5879 | TRAC4_ | mG*mA*mG*rArArUrCrArArArArUrCrGrGrUrGrArArUrGrUrUrUrUrA | 16278 |
| | P12R16_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrArCrArArGrUrCrUrGrUr | |
| | | CrArUrUrCrArCrCrGrArUrUrU*mU*mG*mA | |
| RNACS5880 | TRAC4_ | mG*mA*mG*rArArUrCrArArArArUrCrGrGrUrGrArArUrGrUrUrUrUrA | 16279 |
| | P11R16_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrArCrArArGrUrCrUrGrUr | |
| | | CrArUrUrCrArCrCrGrArUrU*mU*mU*mG | |
| RNACS5881 | TRAC4_ | mG*mA*mG*rArArUrCrArArArArUrCrGrGrUrGrArArUrGrUrUrUrUrA | 16280 |
| | P10R16_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrArCrArArGrUrCrUrGrUr | |
| | | CrArUrUrCrArCrCrGrArU*mU*mU*mU | |
| RNACS5882 | TRAC4_ | mG*mA*mG*rArArUrCrArArArArUrCrGrGrUrGrArArUrGrUrUrUrUrA | 16281 |
| | P9R16_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrArCrArArGrUrCrUrGrUr | |
| | | CrArUrUrCrArCrCrGrA*mU*mU*mU | |
| RNACS5883 | TRAC4_ | mG*mA*mG*rArArUrCrArArArArUrCrGrGrUrGrArArUrGrUrUrUrUrA | 16282 |
| | P8R16_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrArCrArArGrUrCrUrGrUr | |
| | | CrArUrUrCrArCrCrG*mA*mU*mU | |
| RNACS5884 | TRAC4_ | mG*mA*mG*rArArUrCrArArArArUrCrGrGrUrGrArArUrGrUrUrUrUrA | 16283 |
| | P13R18_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrUrGrArCrArArGrUrCrUr | |
| | | GrUrCrArUrUrCrArCrCrGrArUrUrUrUmG*mA*mU | |
| RNACS5885 | TRAC4_ | mG*mA*mG*rArArUrCrArArArArUrCrGrGrUrGrArArUrGrUrUrUrUrA | 16284 |
| | P12R18_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrUrGrArCrArArGrUrCrUr | |
| | | GrUrCrArUrUrCrArCrCrGrArUrUrU*mU*mG*mA | |
| RNACS5886 | TRAC4_ | mG*mA*mG*rArArUrCrArArArArUrCrGrGrUrGrArArUrGrUrUrUrUrA | 16285 |
| | P11R18_del5- | rGrAmGmCmUmAmGmAmAmAmUmAmGmCrArArGrUrUrArArArArUrArArGr | |
| | PAM | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrArGrUrGrArCrArArGrUrCrUr | |
| | | GrUrCrArUrUrCrArCrCrGrArUrU*mU*mU*mG | |
| RNACS5887 | TRAC4_ | mG*mA*mG*rArArUrCrArArArArUrCrGrGrUrGrArArUrGrUrUrUrUrA | 16286 |
| | P10R18_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrUrGrArCrArArGrUrCrUr | |
| | | GrUrCrArUrUrCrArCrCrGrArU*mU*mU*mU | |
| RNACS5888 | TRAC4_ | mG*mA*mG*rArArUrCrArArArArUrCrGrGrUrGrArArUrGrUrUrUrUrA | 16287 |
| | P9R18_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrUrGrArCrArArGrUrCrUr | |
| | | GrUrCrArUrUrCrArCrCrGrA*mU*mU*mU | |
| RNACS5889 | TRAC4_ | mG*mA*mG*rArArUrCrArArArArUrCrGrGrUrGrArArUrGrUrUrUrUrA | 16288 |
| | P8R18_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrUrGrArCrArArGrUrCrUr | |
| | | GrUrCrArUrUrCrArCrCrG*mA*mU*mU | |
| RNACS5890 | TRAC4_ | mG*mA*mG*rArArUrCrArArArArUrCrGrGrUrGrArArUrGrUrUrUrUrA | 16289 |
| | P13R20_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrArGrUrGrArCrArArGrUr | |
| | | CrUrGrUrCrArUrUrCrArCrCrGrArUrUrUrUmG*mA*mU | |
| RNACS5891 | TRAC4_ | mG*mA*mG*rArArUrCrArArArArUrCrGrGrUrGrArArUrGrUrUrUrUrA | 16290 |
| | P12R20_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrArGrUrGrArCrArArGrUr | |
| | | CrUrGrUrCrArUrUrCrArCrCrGrArUrUrU*mU*mG*mA | |
| RNACS5892 | TRAC4_ | mG*mA*mG*rArArUrCrArArArArUrCrGrGrUrGrArArUrGrUrUrUrUrA | 16291 |
| | P11R20_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrArGrUrGrArCrArArGrUr | |
| | | CrUrGrUrCrArUrUrCrArCrCrGrArUrU*mU*mU*mG | |
| RNACS5893 | TRAC4_ | mG*mA*mG*rArArUrCrArArArArUrCrGrGrUrGrArArUrGrUrUrUrUrA | 16292 |
| | P10R20_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrArGrUrGrArCrArArGrUr | |
| | | CrUrGrUrCrArUrUrCrArCrCrGrArU*mU*mU*mU | |
| RNACS5894 | TRAC4_ | mG*mA*mG*rArArUrCrArArArArUrCrGrGrUrGrArArUrGrUrUrUrUrA | 16293 |
| | P9R20_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrArGrUrGrArCrArArGrUr | |
| | | CrUrGrUrCrArUrUrCrArCrCrGrA*mU*mU*mU | |
| RNACS5895 | TRAC4_ | mG*mA*mG*rArArUrCrArArArArUrCrGrGrUrGrArArUrGrUrUrUrUrA | 16294 |
| | P8R20_ | rGrAmGmCmUmAmGmAmAmAmUmAmGmCrArArGrUrUrArArArArUrArArGr | |
| | de15- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrArGrUrGrArCrArArGrUr | |
| | | CrUrGrUrCrArUrUrCrArCrCrG*mA*mU*mU | |
| RNACS5896 | TRAC4_ | mG*mA*mG*rArArUrCrArArArArUrCrGrGrUrGrArArUrGrUrUrUrUrA | 16295 |
| | P13R8_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrUrCrUrGrArUrUrCrArCrCr | |
| | | GrArUrUrUrU*mG*mA*mU | |
| RNACS5897 | TRAC4_ | mG*mA*mG*rArArUrCrArArArArUrCrGrGrUrGrArArUrGrUrUrUrUrA | 16296 |
| | P12R8_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrUrCrUrGrArUrUrCrArCrCr | |
| | | GrArUrUrU*mU*mG*mA | |
| RNACS5898 | TRAC4_ | mG*mA*mG*rArArUrCrArArArArUrCrGrGrUrGrArArUrGrUrUrUrUrA | 16297 |
| | P11R8_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrUrCrUrGrArUrUrCrArCrCr | |
| | | GrArUrU*mU*mU*mG | |
| RNACS5899 | TRAC4_ | mG*mA*mG*rArArUrCrArArArArUrCrGrGrUrGrArArUrGrUrUrUrUrA | 16298 |
| | P10R8_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrUrCrUrGrArUrUrCrArCrCr | |
| | | GrArU*mU*mU*mU | |
| RNACS5900 | TRAC4_ | mG*mA*mG*rArArUrCrArArArArUrCrGrGrUrGrArArUrGrUrUrUrUrA | 16299 |
| | P9R8_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrUrCrUrGrArUrUrCrArCrCr | |
| | | GrA*mU*mU*mU | |
| RNACS5902 | TRAC4_ | mG*mA*mG*rArArUrCrArArArArUrCrGrGrUrGrArArUrGrUrUrUrUrA | 16301 |
| | P13R10_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArArGrUrCrUrGrArUrUrCrAr | |
| | | CrCrGrArUrUrUrU*mG*mA*mU | |
| RNACS5903 | TRAC4_ | mG*mA*mG*rArArUrCrArArArArUrCrGrGrUrGrArArUrGrUrUrUrUrA | 16302 |
| | P12R10_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArArGrUrCrUrGrArUrUrCrAr | |
| | | CrCrGrArUrUrU*mU*mG*mA | |
| RNACS5904 | TRAC4_ | mG*mA*mG*rArArUrCrArArArArUrCrGrGrUrGrArArUrGrUrUrUrUrA | 16303 |
| | P11R10_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArArGrUrCrUrGrArUrUrCrAr | |
| | | CrCrGrArUrU*mU*mU*mG | |
| RNACS5905 | TRAC4_ | mG*mA*mG*rArArUrCrArArArArUrCrGrGrUrGrArArUrGrUrUrUrUrA | 16304 |
| | P10R10_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArArGrUrCrUrGrArUrUrCrAr | |
| | | CrCrGrArU*mU*mU*mU | |
| RNACS5906 | TRAC4_ | mG*mA*mG*rArArUrCrArArArArUrCrGrGrUrGrArArUrGrUrUrUrUrA | 16305 |
| | P9R10_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArArGrUrCrUrGrArUrUrCrAr | |
| | | CrCrGrA*mU*mU*mU | |
| RNACS5907 | TRAC4_ | mG*mA*mG*rArArUrCrArArArArUrCrGrGrUrGrArArUrGrUrUrUrUrA | 16306 |
| | P8R10_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArArGrUrCrUrGrArUrUrCrAr | |
| | | CrCrG*mA*mU*mU | |
| RNACS5908 | TRAC4_ | mG*mA*mG*rArArUrCrArArArArUrCrGrGrUrGrArArUrGrUrUrUrUrA | 16307 |
| | P13R12_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArCrArArGrUrCrUrGrArUrUr | |
| | | CrArCrCrGrArUrUrUrU*mG*mA*mU | |
| RNACS5909 | TRAC4_ | mG*mA*mG*rArArUrCrArArArArUrCrGrGrUrGrArArUrGrUrUrUrUrA | 16308 |
| | P12R12_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArCrArArGrUrCrUrGrArUrUr | |
| | | CrArCrCrGrArUrUrU*mU*mG*mA | |
| RNACS5910 | TRAC4_ | mUmAmGmCrArArGrUrUrArArArArUrArArGrGrCrUrArGrUrCrCrGrUr | 16309 |
| | P11R12_ | UrArUrCrAmAmCmUmUmGmAmAmAmAmAmGmUmGmGmCmAmCmCmGmAmGmUmC | |
| | del7- | mGmGmUmGmCrArCrArArGrUrCrUrGrArUrUrCrArCrCrGrArUrU*mU | |
| | PAM | *mU*mGmG*mA*mG*rArArUrCrArArArArUrCrGrGrUrGrArArUrGrUrU | |
| | | rUrUrArGrAmGmCmUmAmGmAmAmA | |
| RNACS5911 | TRAC4_ | mG*mA*mG*rArArUrCrArArArArUrCrGrGrUrGrArArUrGrUrUrUrUrA | 16310 |
| | P10R12_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArCrArArGrUrCrUrGrArUrUr | |
| | | CrArCrCrGrArU*mU*mU*mU | |
| RNACS5912 | TRAC4_ | mG*mA*mG*rArArUrCrArArArArUrCrGrGrUrGrArArUrGrUrUrUrUrA | 16311 |
| | P9R12_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArCrArArGrUrCrUrGrArUrUr | |
| | | CrArCrCrGrA*mU*mU*mU | |
| RNACS5913 | TRAC4_ | mG*mA*mG*rArArUrCrArArArArUrCrGrGrUrGrArArUrGrUrUrUrUrA | 16312 |
| | P8R12_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArCrArArGrUrCrUrGrArUrUr | |
| | | CrArCrCrG*mA*mU*mU | |
| RNACS5914 | TRAC4_ | mG*mA*mG*rArArUrCrArArArArUrCrGrGrUrGrArArUrGrUrUrUrUrA | 16313 |
| | P13R14_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrArCrArArGrUrCrUrGrAr | |
| | | UrUrCrArCrCrGrArUrUrUrU*mG*mA*mU | |
| RNACS5915 | TRAC4_ | mG*mA*mG*rArArUrCrArArArArUrCrGrGrUrGrArArUrGrUrUrUrUrA | 16314 |
| | P12R14_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrArCrArArGrUrCrUrGrAr | |
| | | UrUrCrArCrCrGrArUrUrU*mU*mG*mA | |
| RNACS5916 | TRAC4_ | mG*mA*mG*rArArUrCrArArArArUrCrGrGrUrGrArArUrGrUrUrUrUrA | 16315 |
| | P11R14_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrArCrArArGrUrCrUrGrAr | |
| | | UrUrCrArCrCrGrArUrU*mU*mU*mG | |
| RNACS5917 | TRAC4_ | mG*mA*mG*rArArUrCrArArArArUrCrGrGrUrGrArArUrGrUrUrUrUrA | 16316 |
| | P10R14_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrArCrArArGrUrCrUrGrAr | |
| | | UrUrCrArCrCrGrArU*mU*mU*mU | |
| RNACS5918 | TRAC4_ | mG*mA*mG*rArArUrCrArArArArUrCrGrGrUrGrArArUrGrUrUrUrUrA | 16317 |
| | P9R14_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrArCrArArGrUrCrUrGrAr | |
| | | UrUrCrArCrCrGrA*mU*mU*mU | |
| RNACS5919 | TRAC4_ | mG*mA*mG*rArArUrCrArArArArUrCrGrGrUrGrArArUrGrUrUrUrUrA | 16318 |
| | P8R14_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrGrArCrArArGrUrCrUrGrAr | |
| | | UrUrCrArCrCrG*mA*mU*mU | |
| RNACS5920 | TRAC4_ | mG*mA*mG*rArArUrCrArArArArUrCrGrGrUrGrArArUrGrUrUrUrUrA | 16319 |
| | P13R16_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrUrGrArCrArArGrUrCrUr | |
| | | GrArUrUrCrArCrCrGrArUrUrUrU*mG*mA*mU | |
| RNACS5921 | TRAC4_ | mG*mA*mG*rArArUrCrArArArArUrCrGrGrUrGrArArUrGrUrUrUrUrA | 16320 |
| | P12R16_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrUrGrArCrArArGrUrCrUr | |
| | | GrArUrUrCrArCrCrGrArUrUrUmU*mG*mA | |
| RNACS5922 | TRAC4_ | mG*mA*mG*rArArUrCrArArArArUrCrGrGrUrGrArArUrGrUrUrUrUrA | 16321 |
| | P11R16_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrUrGrArCrArArGrUrCrUr | |
| | | GrArUrUrCrArCrCrGrArUrU*mU*mU*mG | |
| RNACS5923 | TRAC4_ | mG*mA*mG*rArArUrCrArArArArUrCrGrGrUrGrArArUrGrUrUrUrUrA | 16322 |
| | P10R16_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrUrGrArCrArArGrUrCrUr | |
| | | GrArUrUrCrArCrCrGrArU*mU*mU*mU | |
| RNACS5924 | TRAC4_ | mG*mA*mG*rArArUrCrArArArArUrCrGrGrUrGrArArUrGrUrUrUrUrA | 16323 |
| | POR16_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrUrGrArCrArArGrUrCrUr | |
| | | GrArUrUrCrArCrCrGrA*mU*mU*mU | |
| RNACS5925 | TRAC4_ | mG*mA*mG*rArArUrCrArArArArUrCrGrGrUrGrArArUrGrUrUrUrUrA | 16324 |
| | P8R16_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrUrGrArCrArArGrUrCrUr | |
| | | GrArUrUrCrArCrCrG*mA*mU*mU | |
| RNACS5926 | TRAC4_ | mG*mA*mG*rArArUrCrArArArArUrCrGrGrUrGrArArUrGrUrUrUrUrA | 16325 |
| | P13R18_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrArGrUrGrArCrArArGrUr | |
| | | CrUrGrArUrUrCrArCrCrGrArUrUrUrU*mG*mA*mU | |
| RNACS5927 | TRAC4_ | mG*mA*mG*rArArUrCrArArArArUrCrGrGrUrGrArArUrGrUrUrUrUrA | 16326 |
| | P12R18_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrArGrUrGrArCrArArGrUr | |
| | | CrUrGrArUrUrCrArCrCrGrArUrUrU*mU*mG*mA | |
| RNACS5928 | TRAC4_ | mG*mA*mG*rArArUrCrArArArArUrCrGrGrUrGrArArUrGrUrUrUrUrA | 16327 |
| | P11R18_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrArGrUrGrArCrArArGr | |
| | | UrCrUrGrArUrUrCrArCrCrGrArUrU*mU*mU*mG | |
| RNACS5929 | TRAC4_ | mG*mA*mG*rArArUrCrArArArArUrCrGrGrUrGrArArUrGrUrUrUrUrA | 16328 |
| | P10R18_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrArGrUrGrArCrArArGrUr | |
| | | CrUrGrArUrUrCrArCrCrGrArU*mU*mU*mU | |
| RNACS5930 | TRAC4_ | mG*mA*mG*rArArUrCrArArArArUrCrGrGrUrGrArArUrGrUrUrUrUrA | 16329 |
| | P9R18_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrArGrUrGrArCrArArGrUr | |
| | | CrUrGrArUrUrCrArCrCrGrA*mU*mU*mU | |
| RNACS5931 | TRAC4_ | mG*mA*mG*rArArUrCrArArArArUrCrGrGrUrGrArArUrGrUrUrUrUrA | 16330 |
| | P8R18_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrArGrUrGrArCrArArGrUr | |
| | | CrUrGrArUrUrCrArCrCrG*mA*mU*mU | |
| RNACS5932 | TRAC4_ | mG*mA*mG*rArArUrCrArArArArUrCrGrGrUrGrArArUrGrUrUrUrUrA | 16331 |
| | P13R20_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrCrCrArGrUrGrArCrArAr | |
| | | GrUrCrUrGrArUrUrCrArCrCrGrArUrUrUrU*mG*mA*mU | |
| RNACS5933 | TRAC4_ | mG*mA*mG*rArArUrCrArArArArUrCrGrGrUrGrArArUrGrUrUrUrUrA | 16332 |
| | P12R20_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrCrCrArGrUrGrArCrArAr | |
| | | GrUrCrUrGrArUrUrCrArCrCrGrArUrUrU*mU*mG*mA | |
| RNACS5934 | TRAC4_ | mG*mA*mG*rArArUrCrArArArArUrCrGrGrUrGrArArUrGrUrUrUrUrA | 16333 |
| | P11R20_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrCrCrArGrUrGrArCrArAr | |
| | | GrUrCrUrGrArUrUrCrArCrCrGrArUrU*mU*mU*mG | |
| RNACS5935 | TRAC4_ | mG*mA*mG*rArArUrCrArArArArUrCrGrGrUrGrArArUrGrUrUrUrUrA | 16334 |
| | P10R20_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrCrCrArGrUrGrArCrArAr | |
| | | GrUrCrUrGrArUrUrCrArCrCrGrArU*mU*mU*mU | |
| RNACS5936 | TRAC4_ | mG*mA*mG*rArArUrCrArArArArUrCrGrGrUrGrArArUrGrUrUrUrUrA | 16335 |
| | P9R20_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrCrCrArGrUrGrArCrArAr | |
| | | GrUrCrUrGrArUrUrCrArCrCrGrA*mU*mU*mU | |
| RNACS5937 | TRAC4_ | mG*mA*mG*rArArUrCrArArArArUrCrGrGrUrGrArArUrGrUrUrUrUrA | 16336 |
| | P8R20_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrCrCrArGrUrGrArCrAr | |
| | | ArGrUrCrUrGrArUrUrCrArCrCrG*mA*mU*mU | |
| RNACS5938 | TRAC4_ | mG*mA*mG*rArArUrCrArArArArUrCrGrGrUrGrArArUrGrUrUrUrUrA | 16337 |
| | P13R10_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrCrUrArUrUrUrGrArUrGrCrAr | |
| | | CrCrGrArUrUrUrU*mG*mA*mU | |
| RNACS5939 | TRAC4_ | mG*mA*mG*rArArUrCrArArArArUrCrGrGrUrGrArArUrGrUrUrUrUrA | 16338 |
| | P12R10_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | nick- | mGmCmAmCmCmGmAmGmUmCmGmGmUmGmCrCrUrArUrUrUrGrArUrGrCrAr | |
| | v1 | CrCrGrArUrUrU*mU*mG*mA | |
| RNACS5940 | TRAC4_ | mG*mA*mG*rArArUrCrArArArArUrCrGrGrUrGrArArUrGrUrUrUrUrA | 16339 |
| | P11R10_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | nick- | mGmCmAmCmCmGmAmGmUmCmGmGmUmGmCrCrUrArUrUrUrGrArUrGrCrAr | |
| | v1 | CrCrGrArUrU*mU*mU*mG | |
| RNACS5941 | TRAC4_ | mG*mA*mG*rArArUrCrArArArArUrCrGrGrUrGrArArUrGrUrUrUrUrA | 16340 |
| | P10R10_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | nick- | mGmCmAmCmCmGmAmGmUmCmGmGmUmGmCrCrUrArUrUrUrGrArUrGrCrAr | |
| | | CrCrGrArU*mU*mU*mU | |
| | v1 | | |
| RNACS5942 | TRAC4_ | mG*mA*mG*rArArUrCrArArArArUrCrGrGrUrGrArArUrGrUrUrUrUrA | 16341 |
| | P9R10_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrCrUrArUrUrUrGrArUrGrCrAr | |
| | | CrCrGrA*mU*mU*mU | |
| RNACS5943 | TRAC4_ | mG*mA*mG*rArArUrCrArArArArUrCrGrGrUrGrArArUrGrUrUrUrUrA | 16342 |
| | P8R10_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrCrUrArUrUrUrGrArUrGrCrAr | |
| | | CrCrG*mA*mU*mU | |
| RNACS5944 | TRAC4_ | mG*mA*mG*rArArUrCrArArArArUrCrGrGrUrGrArArUrGrUrUrUrUrA | 16343 |
| | P13R12_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | nick- | mGmCmAmCmCmGmAmGmUmCmGmGmUmGmCrGrCrCrUrArUrUrUrGrArUrGr | |
| | v1 | CrArCrCrGrArUrUrUrU*mG*mA*mU | |
| RNACS5945 | TRAC4_ | mG*mA*mG*rArArUrCrArArArArUrCrGrGrUrGrArArUrGrUrUrUrUrA | 16344 |
| | P12R12_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | nick- | mGmCmAmCmCmGmAmGmUmCmGmGmUmGmCrGrCrCrUrArUrUrUrGrArUrGr | |
| | v1 | CrArCrCrGrArUrUrU*mU*mG*mA | |
| RNACS5946 | TRAC4_ | mG*mA*mG*rArArUrCrArArArArUrCrGrGrUrGrArArUrGrUrUrUrUrA | 16345 |
| | P11R12_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | nick- | mGmCmAmCmCmGmAmGmUmCmGmGmUmGmCrGrCrCrUrArUrUrUrGrArUr | |
| | v1 | GrCrArCrCrGrArUrUmUmUmG | |
| RNACS5947 | TRAC4_ | mG*mA*mG*rArArUrCrArArArArUrCrGrGrUrGrArArUrGrUrUrUrUrA | 16346 |
| | P10R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrGrCrCrUrArUrUrUrGrArUrGr | |
| | | CrArCrCrGrArU*mU*mU*mU | |
| RNACS5948 | TRAC4_ | mG*mA*mG*rArArUrCrArArArArUrCrGrGrUrGrArArUrGrUrUrUrUrA | 16347 |
| | P9R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrGrCrCrUrArUrUrUrGrArUrGr | |
| | | CrArCrCrGrA*mU*mU*mU | |
| RNACS5949 | TRAC4_ | mG*mA*mG*rArArUrCrArArArArUrCrGrGrUrGrArArUrGrUrUrUrUrA | 16348 |
| | P8R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrGrCrCrUrArUrUrUrGrArUrGr | |
| | | CrArCrCrG*mA*mU*mU | |
| RNACS5950 | TRAC4_ | mG*mA*mG*rArArUrCrArArArArUrCrGrGrUrGrArArUrGrUrUrUrUrA | 16349 |
| | P13R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrCrUrGrCrCrUrArUrUrUrGrAr | |
| | | UrGrCrArCrCrGrArUrUrUrU*mG*mA*mU | |
| RNACS5951 | TRAC4_ | mG*mA*mG*rArArUrCrArArArArUrCrGrGrUrGrArArUrGrUrUrUrUrA | 16350 |
| | P12R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrCrUrGrCrCrUrArUrUrUrGrAr | |
| | | UrGrCrArCrCrGrArUrUrU*mU*mG*mA | |
| RNACS5952 | TRAC4_ | mG*mA*mG*rArArUrCrArArArArUrCrGrGrUrGrArArUrGrUrUrUrUrA | 16351 |
| | P11R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrCrUrGrCrCrUrArUrUrUrGrAr | |
| | | UrGrCrArCrCrGrArUrU*mU*mU*mG | |
| RNACS5953 | TRAC4_ | mG*mA*mG*rArArUrCrArArArArUrCrGrGrUrGrArArUrGrUrUrUrUrA | 16352 |
| | P10R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrCrUrGrCrCrUrArUrUrUrGrAr | |
| | | UrGrCrArCrCrGrArU*mU*mU*mU | |
| RNACS5954 | TRAC4_ | mG*mA*mG*rArArUrCrArArArArUrCrGrGrUrGrArArUrGrUrUrUrUrA | 16353 |
| | P9R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrCrUrGrCrCrUrArUrUrUrGrAr | |
| | | UrGrCrArCrCrGrA*mU*mU*mU | |
| RNACS5955 | TRAC4_ | mG*mA*mG*rArArUrCrArArArArUrCrGrGrUrGrArArUrGrUrUrUrUrA | 16354 |
| | P8R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrCrUrGrCrCrUrArUrUrUrGr | |
| | | ArUrGrCrArCrCrG*mA*mU*mU | |
| RNACS5956 | TRAC4_ | mG*mA*mG*rArArUrCrArArArArUrCrGrGrUrGrArArUrGrUrUrUrUrA | 16355 |
| | P13R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrGrUrCrUrGrCrCrUrArUrUrUr | |
| | | GrArUrGrCrArCrCrGrArUrUrUrUmG*mA*mU | |
| RNACS5957 | TRAC4_ | mG*mA*mG*rArArUrCrArArArArUrCrGrGrUrGrArArUrGrUrUrUrUrA | 16356 |
| | P12R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrGrUrCrUrGrCrCrUrArUrUrUr | |
| | | GrArUrGrCrArCrCrGrArUrUrU*mU*mG*mA | |
| RNACS5958 | TRAC4_ | mG*mA*mG*rArArUrCrArArArArUrCrGrGrUrGrArArUrGrUrUrUrUrA | 16357 |
| | P11R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrGrUrCrUrGrCrCrUrArUrUrUr | |
| | | GrArUrGrCrArCrCrGrArUrU*mU*mU*mG | |
| RNACS5959 | TRAC4_ | mG*mA*mG*rArArUrCrArArArArUrCrGrGrUrGrArArUrGrUrUrUrUrA | 16358 |
| | P10R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrGrUrCrUrGrCrCrUrArUrUrUr | |
| | | GrArUrGrCrArCrCrGrArU*mU*mU*mU | |
| RNACS5960 | TRAC4_ | mG*mA*mG*rArArUrCrArArArArUrCrGrGrUrGrArArUrGrUrUrUrUrA | 16359 |
| | POR16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrGrUrCrUrGrCrCrUrArUrUrUr | |
| | | GrArUrGrCrArCrCrGrA*mU*mU*mU | |
| RNACS5961 | TRAC4_ | mG*mA*mG*rArArUrCrArArArArUrCrGrGrUrGrArArUrGrUrUrUrUrA | 16360 |
| | P8R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrGrUrCrUrGrCrCrUrArUrUrUr | |
| | | GrArUrGrCrArCrCrG*mA*mU*mU | |
| RNACS5962 | TRAC4_ | mG*mA*mG*rArArUrCrArArArArUrCrGrGrUrGrArArUrGrUrUrUrUrA | 16361 |
| | P13R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrCrUrGrUrCrUrGrCrCrUrArUr | |
| | | UrUrGrArUrGrCrArCrCrGrArUrUrUrUmG*mA*mU | |
| RNACS5963 | TRAC4_ | mG*mA*mG*rArArUrCrArArArArUrCrGrGrUrGrArArUrGrUrUrUrUrA | 16362 |
| | P12R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrCrUrGrUrCrUrGrCrCrUrArUr | |
| | | UrUrGrArUrGrCrArCrCrGrArUrUrU*mU*mG*mA | |
| RNACS5964 | TRAC4_ | mG*mA*mG*rArArUrCrArArArArUrCrGrGrUrGrArArUrGrUrUrUrUrA | 16363 |
| | P11R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrCrUrGrUrCrUrGrCrCrUrAr | |
| | | UrUrUrGrArUrGrCrArCrCrGrArUrUmU*mU*mG | |
| RNACS5965 | TRAC4_ | mG*mA*mG*rArArUrCrArArArArUrCrGrGrUrGrArArUrGrUrUrUrUrA | 16364 |
| | P10R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrCrUrGrUrCrUrGrCrCrUrArUr | |
| | | UrUrGrArUrGrCrArCrCrGrArU*mU*mU*mU | |
| RNACS5966 | TRAC4_ | mG*mA*mG*rArArUrCrArArArArUrCrGrGrUrGrArArUrGrUrUrUrUrA | 16365 |
| | P9R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrCrUrGrUrCrUrGrCrCrUrArUr | |
| | | UrUrGrArUrGrCrArCrCrGrA*mU*mU*mU | |
| RNACS5967 | TRAC4_ | mG*mA*mG*rArArUrCrArArArArUrCrGrGrUrGrArArUrGrUrUrUrUrA | 16366 |
| | P8R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrCrUrGrUrCrUrGrCrCrUrArUr | |
| | | UrUrGrArUrGrCrArCrCrG*mA*mU*mU | |
| RNACS5968 | TRAC4_ | mG*mA*mG*rArArUrCrArArArArUrCrGrGrUrGrArArUrGrUrUrUrUrA | 16367 |
| | P13R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrGrUrCrUrGrUrCrUrGrCrCrUr | |
| | | ArUrUrUrGrArUrGrCrArCrCrGrArUrUrUrU*mG*mA*mU | |
| RNACS5969 | TRAC4_ | mG*mA*mG*rArArUrCrArArArArUrCrGrGrUrGrArArUrGrUrUrUrUrA | 16368 |
| | P12R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrGrUrCrUrGrUrCrUrGrCrCrUr | |
| | | ArUrUrUrGrArUrGrCrArCrCrGrArUrUrU*mU*mG*mA | |
| RNACS5970 | TRAC4_ | mG*mA*mG*rArArUrCrArArArArUrCrGrGrUrGrArArUrGrUrUrUrUrA | 16369 |
| | P11R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrGrUrCrUrGrUrCrUrGrCrCrUr | |
| | | ArUrUrUrGrArUrGrCrArCrCrGrArUrU*mU*mU*mG | |
| RNACS5971 | TRAC4_ | mG*mA*mG*rArArUrCrArArArArUrCrGrGrUrGrArArUrGrUrUrUrUrA | 16370 |
| | P10R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrGrUrCrUrGrUrCrUrGrCrCrUr | |
| | | ArUrUrUrGrArUrGrCrArCrCrGrArU*mU*mU*mU | |
| RNACS5972 | TRAC4_ | mG*mA*mG*rArArUrCrArArArArUrCrGrGrUrGrArArUrGrUrUrUrUrA | 16371 |
| | P9R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrGrUrCrUrGrUrCrUrGrCrCrUr | |
| | | ArUrUrUrGrArUrGrCrArCrCrGrA*mU*mU*mU | |
| RNACS5973 | TRAC4_ | mG*mA*mG*rArArUrCrArArArArUrCrGrGrUrGrArArUrGrUrUrUrUrA | 16372 |
| | P8R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrGrUrCrUrGrUrCrUrGrCrCrUr | |
| | | ArUrUrUrGrArUrGrCrArCrCrG*mA*mU*mU | |
| RNACS5974 | TRAC4_ | mG*mA*mG*rArArUrCrArArArArUrCrGrGrUrGrArArUrGrUrUrUrUrA | 16373 |
| | P13R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrArArGrUrCrUrGrUrCrUrGrCr | |
| | | CrUrArUrUrUrGrArUrGrCrArCrCrGrArUrUrUrU*mG*mA_ | |
| | | *mU | |
| RNACS5975 | TRAC4_ | mG*mA*mG*rArArUrCrArArArArUrCrGrGrUrGrArArUrGrUrUrUrUrA | 16374 |
| | P12R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrArArGrUrCrUrGrUrCrUrGrCr | |
| | | CrUrArUrUrUrGrArUrGrCrArCrCrGrArUrUrU*mU*mG*mA | |
| RNACS5976 | TRAC4_ | mG*mA*mG*rArArUrCrArArArArUrCrGrGrUrGrArArUrGrUrUrUrUrA | 16375 |
| | P11R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrArArGrUrCrUrGrUrCrUrGrCr | |
| | | CrUrArUrUrUrGrArUrGrCrArCrCrGrArUrUmU*mU*mG | |
| RNACS5977 | TRAC4_ | mG*mA*mG*rArArUrCrArArArArUrCrGrGrUrGrArArUrGrUrUrUrUrA | 16376 |
| | P10R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrArArGrUrCrUrGrUrCrUrGrCr | |
| | | CrUrArUrUrUrGrArUrGrCrArCrCrGrArU*mU*mU*mU | |
| RNACS5978 | TRAC4_ | mG*mA*mG*rArArUrCrArArArArUrCrGrGrUrGrArArUrGrUrUrUrUrA | 16377 |
| | P9R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrArArGrUrCrUrGrUrCrUrGrCr | |
| | | CrUrArUrUrUrGrArUrGrCrArCrCrGrA*mU*mU*mU | |
| RNACS5979 | TRAC4_ | mG*mA*mG*rArArUrCrArArArArUrCrGrGrUrGrArArUrGrUrUrUrUrA | 16378 |
| | P8R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v1 | mGmCmAmCmCmGmAmGmUmCmGmGmUmGmCrArArGrUrCrUrGrUrCrUrGrCr | |
| | | CrUrArUrUrUrGrArUrGrCrArCrCrG*mA*mU*mU | |
| RNACS5980 | TRAC4_ | mG*mA*mG*rArArUrCrArArArArUrCrGrGrUrGrArArUrGrUrUrUrUrA | 16379 |
| | P13R10_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrCrUrArUrUrUrArArCrCrCrAr | |
| | | CrCrGrArUrUrUrU*mG*mA*mU | |
| RNACS5981 | TRAC4_ | mG*mA*mG*rArArUrCrArArArArUrCrGrGrUrGrArArUrGrUrUrUrUrA | 16380 |
| | P12R10_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrCrUrArUrUrUrArArCrCrCrAr | |
| | | CrCrGrArUrUrU*mU*mG*mA | |
| RNACS5982 | TRAC4_ | mG*mA*mG*rArArUrCrArArArArUrCrGrGrUrGrArArUrGrUrUrUrUrA | 16381 |
| | P11R10_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUm | |
| | nick-v2 | GmGmCmAmCmCmGmAmGmUmCmGmGmUmGmCrCrUrArUrUrUrArArCrCrCr | |
| | | ArCrCrGrArUrUmUmU*mG | |
| RNACS5983 | TRAC4_ | mG*mA*mG*rArArUrCrArArArArUrCrGrGrUrGrArArUrGrUrUrUrUrA | 16382 |
| | P10R10_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrCrUrArUrUrUrArArCrCrCrAr | |
| | | CrCrGrArU*mU*mU*mU | |
| RNACS5984 | TRAC4_ | mG*mA*mG*rArArUrCrArArArArUrCrGrGrUrGrArArUrGrUrUrUrUrA | 16383 |
| | P9R10_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrCrUrArUrUrUrArArCrCrCrAr | |
| | | CrCrGrA*mU*mU*mU | |
| RNACS5985 | TRAC4_ | mG*mA*mG*rArArUrCrArArArArUrCrGrGrUrGrArArUrGrUrUrUrUrA | 16384 |
| | P8R10_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrCrUrArUrUrUrArArCrCrCrAr | |
| | | CrCrG*mA*mU*mU | |
| RNACS5986 | TRAC4_ | mG*mA*mG*rArArUrCrArArArArUrCrGrGrUrGrArArUrGrUrUrUrUrA | 16385 |
| | P13R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrGrCrCrUrArUrUrUrArArCrCr | |
| | | CrArCrCrGrArUrUrUrU*mG*mA*mU | |
| RNACS5987 | TRAC4_ | mG*mA*mG*rArArUrCrArArArArUrCrGrGrUrGrArArUrGrUrUrUrUrA | 16386 |
| | P12R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrGrCrCrUrArUrUrUrArArCrCr | |
| | | CrArCrCrGrArUrUrU*mU*mG*mA | |
| RNACS5988 | TRAC4_ | mG*mA*mG*rArArUrCrArArArArUrCrGrGrUrGrArArUrGrUrUrUrUrA | 16387 |
| | P11R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrGrCrCrUrArUrUrUrArArCrCr | |
| | | CrArCrCrGrArUrU*mU*mU*mG | |
| RNACS5989 | TRAC4_ | mG*mA*mG*rArArUrCrArArArArUrCrGrGrUrGrArArUrGrUrUrUrUrA | 16388 |
| | P10R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrGrCrCrUrArUrUrUrArArCrCr | |
| | | CrArCrCrGrArU*mU*mU*mU | |
| RNACS5990 | TRAC4_ | mG*mA*mG*rArArUrCrArArArArUrCrGrGrUrGrArArUrGrUrUrUrUrA | 16389 |
| | P9R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrGrCrCrUrArUrUrUrArArCrCr | |
| | | CrArCrCrGrA*mU*mU*mU | |
| RNACS5991 | TRAC4_ | mG*mA*mG*rArArUrCrArArArArUrCrGrGrUrGrArArUrGrUrUrUrUrA | 16390 |
| | P8R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrGrCrCrUrArUrUrUrArArCrCr | |
| | | CrArCrCrG*mA*mU*mU | |
| RNACS5992 | TRAC4_ | mG*mA*mG*rArArUrCrArArArArUrCrGrGrUrGrArArUrGrUrUrUrUrA | 16391 |
| | P13R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrCrUrGrCrCrUrArUrUrUrArAr | |
| | | CrCrCrArCrCrGrArUrUrUrU*mG*mA*mU | |
| RNACS5993 | TRAC4_ | mG*mA*mG*rArArUrCrArArArArUrCrGrGrUrGrArArUrGrUrUrUrUrA | 16392 |
| | P12R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrCrUrGrCrCrUrArUrUrUrArAr | |
| | | CrCrCrArCrCrGrArUrUrU*mU*mG*mA | |
| RNACS5994 | TRAC4_ | mG*mA*mG*rArArUrCrArArArArUrCrGrGrUrGrArArUrGrUrUrUrUrA | 16393 |
| | P11R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrCrUrGrCrCrUrArUrUrUrArAr | |
| | | CrCrCrArCrCrGrArUrU*mU*mU*mG | |
| RNACS5995 | TRAC4_ | mG*mA*mG*rArArUrCrArArArArUrCrGrGrUrGrArArUrGrUrUrUrUrA | 16394 |
| | P10R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrCrUrGrCrCrUrArUrUrUrArAr | |
| | | CrCrCrArCrCrGrArU*mU*mU*mU | |
| RNACS5996 | TRAC4_ | mG*mA*mG*rArArUrCrArArArArUrCrGrGrUrGrArArUrGrUrUrUrUrA | 16395 |
| | P9R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrCrUrGrCrCrUrArUrUrUrArAr | |
| | | CrCrCrArCrCrGrA*mU*mU*mU | |
| RNACS5997 | TRAC4_ | mG*mA*mG*rArArUrCrArArArArUrCrGrGrUrGrArArUrGrUrUrUrUrA | 16396 |
| | P8R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrCrUrGrCrCrUrArUrUrUrArAr | |
| | | CrCrCrArCrCrG*mA*mU*mU | |
| RNACS5998 | TRAC4_ | mG*mA*mG*rArArUrCrArArArArUrCrGrGrUrGrArArUrGrUrUrUrUrA | 16397 |
| | P13R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrGrUrCrUrGrCrCrUrArUrUrUr | |
| | | ArArCrCrCrArCrCrGrArUrUrUrU*mG*mA*mU | |
| RNACS5999 | TRAC4_ | mG*mA*mG*rArArUrCrArArArArUrCrGrGrUrGrArArUrGrUrUrUrUrA | 16398 |
| | P12R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrGrUrCrUrGrCrCrUrArUrUrUr | |
| | | ArArCrCrCrArCrCrGrArUrUrU*mU*mG*mA | |
| RNACS6000 | TRAC4_ | mG*mA*mG*rArArUrCrArArArArUrCrGrGrUrGrArArUrGrUrUrUrUrA | 16399 |
| | P11R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrGrUrCrUrGrCrCrUrArUrUr | |
| | | UrArArCrCrCrArCrCrGrArUrUmU*mUmG | |
| RNACS6001 | TRAC4_ | mG*mA*mG*rArArUrCrArArArArUrCrGrGrUrGrArArUrGrUrUrUrUrA | 16400 |
| | P10R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrGrUrCrUrGrCrCrUrArUrUrUr | |
| | | ArArCrCrCrArCrCrGrArU*mU*mU*mU | |
| RNACS6002 | TRAC4_ | mG*mA*mG*rArArUrCrArArArArUrCrGrGrUrGrArArUrGrUrUrUrUrA | 16401 |
| | P9R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrGrUrCrUrGrCrCrUrArUrUrUr | |
| | | ArArCrCrCrArCrCrGrA*mU*mU*mU | |
| RNACS6003 | TRAC4_ | mG*mA*mG*rArArUrCrArArArArUrCrGrGrUrGrArArUrGrUrUrUrUrA | 16402 |
| | P8R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrGrUrCrUrGrCrCrUrArUrUrUr | |
| | | ArArCrCrCrArCrCrG*mA*mU*mU | |
| RNACS6004 | TRAC4_ | mG*mA*mG*rArArUrCrArArArArUrCrGrGrUrGrArArUrGrUrUrUrUrA | 16403 |
| | P13R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrCrUrGrUrCrUrGrCrCrUrArUr | |
| | | UrUrArArCrCrCrArCrCrGrArUrUrUrU*mG*mA*mU | |
| RNACS6005 | TRAC4_ | mG*mA*mG*rArArUrCrArArArArUrCrGrGrUrGrArArUrGrUrUrUrUrA | 16404 |
| | P12R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrCrUrGrUrCrUrGrCrCrUrArUr | |
| | | UrUrArArCrCrCrArCrCrGrArUrUrU*mU*mG*mA | |
| RNACS6006 | TRAC4_ | mG*mA*mG*rArArUrCrArArArArUrCrGrGrUrGrArArUrGrUrUrUrUrA | 16405 |
| | P11R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrCrUrGrUrCrUrGrCrCrUrArUr | |
| | | UrUrArArCrCrCrArCrCrGrArUrU*mU*mU*mG | |
| RNACS6007 | TRAC4_ | mG*mA*mG*rArArUrCrArArArArUrCrGrGrUrGrArArUrGrUrUrUrUrA | 16406 |
| | P10R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrCrUrGrUrCrUrGrCrCrUrArUr | |
| | | UrUrArArCrCrCrArCrCrGrArU*mU*mU*mU | |
| RNACS6008 | TRAC4_ | mG*mA*mG*rArArUrCrArArArArUrCrGrGrUrGrArArUrGrUrUrUrUrA | 16407 |
| | P9R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrCrUrGrUrCrUrGrCrCrUrArUr | |
| | | UrUrArArCrCrCrArCrCrGrA*mU*mU*mU | |
| RNACS6009 | TRAC4_ | mG*mA*mG*rArArUrCrArArArArUrCrGrGrUrGrArArUrGrUrUrUrUrA | 16408 |
| | P8R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrCrUrGrUrCrUrGrCrCrUrAr | |
| | | UrUrUrArArCrCrCrArCrCrG*mA*mU*mU | |
| RNACS6010 | TRAC4_ | mG*mA*mG*rArArUrCrArArArArUrCrGrGrUrGrArArUrGrUrUrUrUrA | 16409 |
| | P13R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrGrUrCrUrGrUrCrUrGrCrCrUr | |
| | | ArUrUrUrArArCrCrCrArCrCrGrArUrUrUrUmG*mA*mU | |
| RNACS6011 | TRAC4_ | mG*mA*mG*rArArUrCrArArArArUrCrGrGrUrGrArArUrGrUrUrUrUrA | 16410 |
| | P12R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrGrUrCrUrGrUrCrUrGrCrCrUr | |
| | | ArUrUrUrArArCrCrCrArCrCrGrArUrUrU*mU*mG*mA | |
| RNACS6012 | TRAC4_ | mG*mA*mG*rArArUrCrArArArArUrCrGrGrUrGrArArUrGrUrUrUrUrA | 16411 |
| | P11R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrGrUrCrUrGrUrCrUrGrCrCrUr | |
| | | ArUrUrUrArArCrCrCrArCrCrGrArUrU*mU*mU*mG | |
| RNACS6013 | TRAC4_ | mG*mA*mG*rArArUrCrArArArArUrCrGrGrUrGrArArUrGrUrUrUrUrA | 16412 |
| | P10R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrGrUrCrUrGrUrCrUrGrCrCrUr | |
| | | ArUrUrUrArArCrCrCrArCrCrGrArU*mU*mU*mU | |
| RNACS6014 | TRAC4_ | mG*mA*mG*rArArUrCrArArArArUrCrGrGrUrGrArArUrGrUrUrUrUrA | 16413 |
| | P9R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrGrUrCrUrGrUrCrUrGrCrCrUr | |
| | | ArUrUrUrArArCrCrCrArCrCrGrA*mU*mU*mU | |
| RNACS6015 | TRAC4_ | mG*mA*mG*rArArUrCrArArArArUrCrGrGrUrGrArArUrGrUrUrUrUrA | 16414 |
| | P8R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrGrUrCrUrGrUrCrUrGrCrCrUr | |
| | | ArUrUrUrArArCrCrCrArCrCrG*mA*mU*mU | |
| RNACS6016 | TRAC4_ | mG*mA*mG*rArArUrCrArArArArUrCrGrGrUrGrArArUrGrUrUrUrUrA | 16415 |
| | P13R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrArArGrUrCrUrGrUrCrUrGrCr | |
| | | CrUrArUrUrUrArArCrCrCrArCrCrGrArUrUrUrUmG*mA*mU | |
| RNACS6017 | TRAC4_ | mG*mA*mG*rArArUrCrArArArArUrCrGrGrUrGrArArUrGrUrUrUrUrA | 16416 |
| | P12R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrArArGrUrCrUrGrUrCrUrGrCr | |
| | | CrUrArUrUrUrArArCrCrCrArCrCrGrArUrUrU*mU*mG*mA | |
| RNACS6018 | TRAC4_ | mG*mA*mG*rArArUrCrArArArArUrCrGrGrUrGrArArUrGrUrUrUrUrA | 16417 |
| | P11R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrArArGrUrCrUrGrUrCrUrGrCr | |
| | | CrUrArUrUrUrArArCrCrCrArCrCrGrArUrU*mU*mU*mG | |
| RNACS6019 | TRAC4_ | mG*mA*mG*rArArUrCrArArArArUrCrGrGrUrGrArArUrGrUrUrUrUrA | 16418 |
| | P10R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrArArGrUrCrUrGrUrCrUrGrCr | |
| | | CrUrArUrUrUrArArCrCrCrArCrCrGrArU*mU*mU*mU | |
| RNACS6020 | TRAC4_ | mG*mA*mG*rArArUrCrArArArArUrCrGrGrUrGrArArUrGrUrUrUrUrA | 16419 |
| | P9R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrArArGrUrCrUrGrUrCrUrGrCr | |
| | | CrUrArUrUrUrArArCrCrCrArCrCrGrA*mU*mU*mU | |
| RNACS6021 | TRAC4_ | mG*mA*mG*rArArUrCrArArArArUrCrGrGrUrGrArArUrGrUrUrUrUrA | 16420 |
| | P8R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick-v2 | mGmCmAmCmCmGmAmGmUmCmGmGmUmGmCrArArGrUrCrUrGrUrCrUrGrCr | |
| | | CrUrArUrUrUrArArCrCrCrArCrCrG*mA*mU*mU | |
| RNACS6022 | TRAC5_ | mG*mC*mU*rGrGrUrArCrArCrGrGrCrArGrGrGrUrCrArGrUrUrUrUrA | 16421 |
| | P13R10_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrUrGrArUrArGrUrGrCrCr | |
| | | CrUrGrCrCrGrUrG*mU*mA*mC | |
| RNACS6023 | TRAC5_ | mG*mC*mU*rGrGrUrArCrArCrGrGrCrArGrGrGrUrCrArGrUrUrUrUrA | 16422 |
| | P12R10_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrUrGrArUrArGrUrGrCrCr | |
| | | CrUrGrCrCrGrU*mG*mU*mA | |
| RNACS6024 | TRAC5_ | mG*mC*mU*rGrGrUrArCrArCrGrGrCrArGrGrGrUrCrArGrUrUrUrUrA | 16423 |
| | P11R10_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrUrGrArUrArGrUrGrCrCr | |
| | | CrUrGrCrCrG*mU*mG*mU | |
| RNACS6025 | TRAC5_ | mG*mC*mU*rGrGrUrArCrArCrGrGrCrArGrGrGrUrCrArGrUrUrUrUrA | 16424 |
| | P10R10_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrUrGrArUrArGrUrGrCrCr | |
| | | CrUrGrCrC*mG*mU*mG | |
| RNACS6026 | TRAC5_ | mG*mC*mU*rGrGrUrArCrArCrGrGrCrArGrGrGrUrCrArGrUrUrUrUrA | 16425 |
| | P9R10_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrUrGrArUrArGrUrGrCrCr | |
| | | CrUrGrC*mC*mG*mU | |
| RNACS6027 | TRAC5_ | mG*mC*mU*rGrGrUrArCrArCrGrGrCrArGrGrGrUrCrArGrUrUrUrUrA | 16426 |
| | P8R10_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrUrGrArUrArGrUrGrCrCr | |
| | | CrUrG*mC*mC*mG | |
| RNACS6028 | TRAC5_ | mG*mC*mU*rGrGrUrArCrArCrGrGrCrArGrGrGrUrCrArGrUrUrUrUrA | 16427 |
| | P13R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArCrCrCrUrGrArUrArGrUrGr | |
| | | CrCrCrUrGrCrCrGrUrG*mU*mA*mC | |
| RNACS6029 | TRAC5_ | mG*mC*mU*rGrGrUrArCrArCrGrGrCrArGrGrGrUrCrArGrUrUrUrUrA | 16428 |
| | P12R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArCrCrCrUrGrArUrArGrUrGr | |
| | | CrCrCrUrGrCrCrGrU*mG*mU*mA | |
| RNACS6030 | TRAC5_ | mG*mC*mU*rGrGrUrArCrArCrGrGrCrArGrGrGrUrCrArGrUrUrUrUrA | 16429 |
| | P11R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArCrCrCrUrGrArUrArGrUrGr | |
| | | CrCrCrUrGrCrCrG*mU*mG*mU | |
| RNACS6031 | TRAC5_ | mG*mC*mU*rGrGrUrArCrArCrGrGrCrArGrGrGrUrCrArGrUrUrUrUrA | 16430 |
| | P10R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArCrCrCrUrGrArUrArGrUrGr | |
| | | CrCrCrUrGrCrC*mG*mU*mG | |
| RNACS6032 | TRAC5_ | mG*mC*mU*rGrGrUrArCrArCrGrGrCrArGrGrGrUrCrArGrUrUrUrUrA | 16431 |
| | P9R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArCrCrCrUrGrArUrArGrUrGr | |
| | | CrCrCrUrGrC*mC*mG*mU | |
| RNACS6033 | TRAC5_ | mG*mC*mU*rGrGrUrArCrArCrGrGrCrArGrGrGrUrCrArGrUrUrUrUrA | 16432 |
| | P8R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArCrCrCrUrGrArUrArGrUrGr | |
| | | CrCrCrUrG*mC*mC*mG | |
| RNACS6034 | TRAC5_ | mG*mC*mU*rGrGrUrArCrArCrGrGrCrArGrGrGrUrCrArGrUrUrUrUrA | 16433 |
| | P13R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArArCrCrCrUrGrArUrArGr | |
| | | UrGrCrCrCrUrGrCrCrGrUrG*mU*mA*mC | |
| RNACS6035 | TRAC5_ | mG*mC*mU*rGrGrUrArCrArCrGrGrCrArGrGrGrUrCrArGrUrUrUrUrA | 16434 |
| | P12R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArArCrCrCrUrGrArUrArGr | |
| | | UrGrCrCrCrUrGrCrCrGrU*mG*mU*mA | |
| RNACS6036 | TRAC5_ | mG*mC*mU*rGrGrUrArCrArCrGrGrCrArGrGrGrUrCrArGrUrUrUrUrA | 16435 |
| | P11R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArArCrCrCrUrGrArUrArGr | |
| | | UrGrCrCrCrUrGrCrCrG*mU*mG*mU | |
| RNACS6037 | TRAC5_ | mG*mC*mU*rGrGrUrArCrArCrGrGrCrArGrGrGrUrCrArGrUrUrUrUrA | 16436 |
| | P10R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArArCrCrCrUrGrArUrArGr | |
| | | UrGrCrCrCrUrGrCrC*mG*mU*mG | |
| RNACS6038 | TRAC5_ | mG*mC*mU*rGrGrUrArCrArCrGrGrCrArGrGrGrUrCrArGrUrUrUrUrA | 16437 |
| | P9R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArArCrCrCrUrGrArUrArGr | |
| | | UrGrCrCrCrUrGrC*mC*mG*mU | |
| RNACS6039 | TRAC5_ | mG*mC*mU*rGrGrUrArCrArCrGrGrCrArGrGrGrUrCrArGrUrUrUrUrA | 16438 |
| | P8R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArArCrCrCrUrGrArUrArGr | |
| | | UrGrCrCrCrUrG*mC*mC*mG | |
| RNACS6040 | TRAC5_ | mG*mC*mU*rGrGrUrArCrArCrGrGrCrArGrGrGrUrCrArGrUrUrUrUrA | 16439 |
| | P13R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArGrArArCrCrCrUrGrArUr | |
| | | ArGrUrGrCrCrCrUrGrCrCrGrUrG*mU*mA*mC | |
| RNACS6041 | TRAC5_ | mG*mC*mU*rGrGrUrArCrArCrGrGrCrArGrGrGrUrCrArGrUrUrUrUrA | 16440 |
| | P12R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArGrArArCrCrCrUrGrArUr | |
| | | ArGrUrGrCrCrCrUrGrCrCrGrU*mG*mU*mA | |
| RNACS6042 | TRAC5_ | mG*mC*mU*rGrGrUrArCrArCrGrGrCrArGrGrGrUrCrArGrUrUrUrUrA | 16441 |
| | P11R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArGrArArCrCrCrUrGrArUr | |
| | | ArGrUrGrCrCrCrUrGrCrCrG*mU*mG*mU | |
| RNACS6043 | TRAC5_ | mG*mC*mU*rGrGrUrArCrArCrGrGrCrArGrGrGrUrCrArGrUrUrUrUrA | 16442 |
| | P10R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArGrArArCrCrCrUrGrArUr | |
| | | ArGrUrGrCrCrCrUrGrCrC*mG*mU*mG | |
| RNACS6044 | TRAC5_ | mG*mC*mU*rGrGrUrArCrArCrGrGrCrArGrGrGrUrCrArGrUrUrUrUrA | 16443 |
| | P9R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArGrArArCrCrCrUrGrArUr | |
| | | ArGrUrGrCrCrCrUrGrC*mC*mG*mU | |
| RNACS6045 | TRAC5_ | mG*mC*mU*rGrGrUrArCrArCrGrGrCrArGrGrGrUrCrArGrUrUrUrUrA | 16444 |
| | P8R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArGrArArCrCrCrUrGrArUrA | |
| | | rGrUrGrCrCrCrUrG*mC*mC*mG | |
| RNACS6046 | TRAC5_ | mG*mC*mU*rGrGrUrArCrArCrGrGrCrArGrGrGrUrCrArGrUrUrUrUrA | 16445 |
| | P13R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrCrCrArGrArArCrCrCrUrGr | |
| | | ArUrArGrUrGrCrCrCrUrGrCrCrGrUrG*mU*mA*mC | |
| RNACS6047 | TRAC5_ | mG*mC*mU*rGrGrUrArCrArCrGrGrCrArGrGrGrUrCrArGrUrUrUrUrA | 16446 |
| | P12R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrCrCrArGrArArCrCrCrUrGr | |
| | | ArUrArGrUrGrCrCrCrUrGrCrCrGrU*mG*mU*mA | |
| RNACS6048 | TRAC5_ | mG*mC*mU*rGrGrUrArCrArCrGrGrCrArGrGrGrUrCrArGrUrUrUrUrA | 16447 |
| | P11R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrCrCrArGrArArCrCrCrUrGr | |
| | | ArUrArGrUrGrCrCrCrUrGrCrCrG*mU*mG*mU | |
| RNACS6049 | TRAC5_ | mG*mC*mU*rGrGrUrArCrArCrGrGrCrArGrGrGrUrCrArGrUrUrUrUrA | 16448 |
| | P10R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrCrCrArGrArArCrCrCrUrGr | |
| | | ArUrArGrUrGrCrCrCrUrGrCrC*mG*mU*mG | |
| RNACS6050 | TRAC5_ | mG*mC*mU*rGrGrUrArCrArCrGrGrCrArGrGrGrUrCrArGrUrUrUrUrA | 16449 |
| | P9R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrCrCrArGrArArCrCrCrUrGr | |
| | | ArUrArGrUrGrCrCrCrUrGrC*mC*mG*mU | |
| RNACS6051 | TRAC5_ | mG*mC*mU*rGrGrUrArCrArCrGrGrCrArGrGrGrUrCrArGrUrUrUrUrA | 16450 |
| | P8R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrCrCrArGrArArCrCrCrUrGr | |
| | | ArUrArGrUrGrCrCrCrUrG*mC*mC*mG | |
| RNACS6052 | TRAC5_ | mG*mC*mU*rGrGrUrArCrArCrGrGrCrArGrGrGrUrCrArGrUrUrUrUrA | 16451 |
| | P13R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrArUrCrCrArGrArArCrCrCr | |
| | | UrGrArUrArGrUrGrCrCrCrUrGrCrCrGrUrG*mU*mA*mC | |
| RNACS6053 | TRAC5_ | mG*mC*mU*rGrGrUrArCrArCrGrGrCrArGrGrGrUrCrArGrUrUrUrUrA | 16452 |
| | P12R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrArUrCrCrArGrArArCrCrCr | |
| | | UrGrArUrArGrUrGrCrCrCrUrGrCrCrGrU*mG*mU*mA | |
| RNACS6054 | TRAC5_ | mG*mC*mU*rGrGrUrArCrArCrGrGrCrArGrGrGrUrCrArGrUrUrUrUrA | 16453 |
| | P11R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrArUrCrCrArGrArArCrCrCr | |
| | | UrGrArUrArGrUrGrCrCrCrUrGrCrCrG*mU*mG*mU | |
| RNACS6055 | TRAC5_ | mG*mC*mU*rGrGrUrArCrArCrGrGrCrArGrGrGrUrCrArGrUrUrUrUrA | 16454 |
| | P10R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrArUrCrCrArGrArArCrCrCr | |
| | | UGrArUrArGrUrGrCrCrCrUrGrCrC*mG*mU*mG | |
| RNACS6056 | TRAC5_ | mG*mC*mU*rGrGrUrArCrArCrGrGrCrArGrGrGrUrCrArGrUrUrUrUrA | 16455 |
| | P9R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrArUrCrCrArGrArArCrCrCr | |
| | | UrGrArUrArGrUrGrCrCrCrUrGrC*mC*mG*mU | |
| RNACS6057 | TRAC5_ | mG*mC*mU*rGrGrUrArCrArCrGrGrCrArGrGrGrUrCrArGrUrUrUrUrA | 16456 |
| | P8R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrArUrCrCrArGrArArCrCrCr | |
| | | UrGrArUrArGrUrGrCrCrCrUrG*mC*mC*mG | |
| RNACS6058 | TRAC5_ | mG*mC*mU*rGrGrUrArCrArCrGrGrCrArGrGrGrUrCrArGrUrUrUrUrA | 16457 |
| | P13R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArUrArUrCrCrArGrArArCr | |
| | | CrCrUrGrArUrArGrUrGrCrCrCrUrGrCrCrGrUrG*mU*mA*mC | |
| RNACS6059 | TRAC5_ | mG*mC*mU*rGrGrUrArCrArCrGrGrCrArGrGrGrUrCrArGrUrUrUrUrA | 16458 |
| | P12R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArUrArUrCrCrArGrArArCr | |
| | | CrCrUrGrArUrArGrUrGrCrCrCrUrGrCrCrGrU*mG*mU*mA | |
| RNACS6060 | TRAC5_ | mG*mC*mU*rGrGrUrArCrArCrGrGrCrArGrGrGrUrCrArGrUrUrUrUrA | 16459 |
| | P11R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArUrArUrCrCrArGrArArCr | |
| | | CrCrUrGrArUrArGrUrGrCrCrCrUrGrCrCrG*mU*mG*mU | |
| RNACS6061 | TRAC5_ | mG*mC*mU*rGrGrUrArCrArCrGrGrCrArGrGrGrUrCrArGrUrUrUrUrA | 16460 |
| | P10R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArUrArUrCrCrArGrArArCr | |
| | | CrCrUrGrArUrArGrUrGrCrCrCrUrGrCrC*mG*mU*mG | |
| RNACS6062 | TRAC5_ | mG*mC*mU*rGrGrUrArCrArCrGrGrCrArGrGrGrUrCrArGrUrUrUrUrA | 16461 |
| | P9R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArUrArUrCrCrArGrArArCr | |
| | | CrCrUrGrArUrArGrUrGrCrCrCrUrGrC*mC*mG*mU | |
| RNACS6063 | TRAC5_ | mG*mC*mU*rGrGrUrArCrArCrGrGrCrArGrGrGrUrCrArGrUrUrUrUrA | 16462 |
| | P8R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArUrArUrCrCrArGrArArCr | |
| | | CrCrUrGrArUrArGrUrGrCrCrCrUrG*mC*mC*mG | |
| RNACS6064 | TRAC5_ | mG*mC*mU*rGrGrUrArCrArCrGrGrCrArGrGrGrUrCrArGrUrUrUrUrA | 16463 |
| | P13R12_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrUrGrArUrArGrUrUrGrAr | |
| | | CrCrCrUrGrCrCrGrUrG*mU*mA*mC | |
| RNACS6065 | TRAC5_ | mG*mC*mU*rGrGrUrArCrArCrGrGrCrArGrGrGrUrCrArGrUrUrUrUrA | 16464 |
| | P12R12_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrUrGrArUrArGrUrUrGrAr | |
| | | CrCrCrUrGrCrCrGrU*mG*mU*mA | |
| RNACS6066 | TRAC5_ | mG*mC*mU*rGrGrUrArCrArCrGrGrCrArGrGrGrUrCrArGrUrUrUrUrA | 16465 |
| | P11R12_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrUrGrArUrArGrUrUrGrAr | |
| | | CrCrCrUrGrCrCrG*mU*mG*mU | |
| RNACS6067 | TRAC5_ | mG*mC*mU*rGrGrUrArCrArCrGrGrCrArGrGrGrUrCrArGrUrUrUrUrA | 16466 |
| | P10R12_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrUrGrArUrArGrUrUrGrAr | |
| | | CrCrCrUrGrCrC*mG*mU*mG | |
| RNACS6068 | TRAC5_ | mG*mC*mU*rGrGrUrArCrArCrGrGrCrArGrGrGrUrCrArGrUrUrUrUrA | 16467 |
| | P9R12_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrUrGrArUrArGrUrUrGrAr | |
| | | CrCrCrUrGrC*mC*mG*mU | |
| RNACS6069 | TRAC5_ | mG*mC*mU*rGrGrUrArCrArCrGrGrCrArGrGrGrUrCrArGrUrUrUrUrA | 16468 |
| | P8R12_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrUrGrArUrArGrUrUrGrAr | |
| | | CrCrCrUrG*mC*mC*mG | |
| RNACS6070 | TRAC5_ | mG*mC*mU*rGrGrUrArCrArCrGrGrCrArGrGrGrUrCrArGrUrUrUrUrA | 16469 |
| | P13R14_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArCrCrCrUrGrArUrArGrUrUr | |
| | | GrArCrCrCrUrGrCrCrGrUrG*mU*mA*mC | |
| RNACS6071 | TRAC5_ | mG*mC*mU*rGrGrUrArCrArCrGrGrCrArGrGrGrUrCrArGrUrUrUrUrA | 16470 |
| | P12R14_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArCrCrCrUrGrArUrArGrUrUr | |
| | | GrArCrCrCrUrGrCrCrGrU*mG*mU*mA | |
| RNACS6072 | TRAC5_ | mG*mC*mU*rGrGrUrArCrArCrGrGrCrArGrGrGrUrCrArGrUrUrUrUrA | 16471 |
| | P11R14_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArCrCrCrUrGrArUrArGrUrUr | |
| | | GrArCrCrCrUrGrCrCrG*mU*mG*mU | |
| RNACS6073 | TRAC5_ | mG*mC*mU*rGrGrUrArCrArCrGrGrCrArGrGrGrUrCrArGrUrUrUrUrA | 16472 |
| | P10R14_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArCrCrCrUrGrArUrArGrUrUr | |
| | | GrArCrCrCrUrGrCrC*mG*mU*mG | |
| RNACS6074 | TRAC5_ | mG*mC*mU*rGrGrUrArCrArCrGrGrCrArGrGrGrUrCrArGrUrUrUrUrA | 16473 |
| | P9R14_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArCrCrCrUrGrArUrArGrUrUr | |
| | | GrArCrCrCrUrGrC*mC*mG*mU | |
| RNACS6075 | TRAC5_ | mG*mC*mU*rGrGrUrArCrArCrGrGrCrArGrGrGrUrCrArGrUrUrUrUrA | 16474 |
| | P8R14_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArCrCrCrUrGrArUrArGrUrUr | |
| | | GrArCrCrCrUrG*mC*mC*mG | |
| RNACS6076 | TRAC5_ | mG*mC*mU*rGrGrUrArCrArCrGrGrCrArGrGrGrUrCrArGrUrUrUrUrA | 16475 |
| | P13R16_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArArCrCrCrUrGrArUrArGr | |
| | | UrUrGrArCrCrCrUrGrCrCrGrUrG*mU*mA*mC | |
| RNACS6077 | TRAC5_ | mG*mC*mU*rGrGrUrArCrArCrGrGrCrArGrGrGrUrCrArGrUrUrUrUrA | 16476 |
| | P12R16_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArArCrCrCrUrGrArUrArGr | |
| | | UrUrGrArCrCrCrUrGrCrCrGrU*mG*mU*mA | |
| RNACS6078 | TRAC5_ | mG*mC*mU*rGrGrUrArCrArCrGrGrCrArGrGrGrUrCrArGrUrUrUrUrA | 16477 |
| | P11R16_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArArCrCrCrUrGrArUrArGr | |
| | | UrUrGrArCrCrCrUrGrCrCrG*mU*mG*mU | |
| RNACS6079 | TRAC5_ | mG*mC*mU*rGrGrUrArCrArCrGrGrCrArGrGrGrUrCrArGrUrUrUrUrA | 16478 |
| | P10R16_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArArCrCrCrUrGrArUrArGr | |
| | | UrUrGrArCrCrCrUrGrCrC*mG*mU*mG | |
| RNACS6080 | TRAC5_ | mG*mC*mU*rGrGrUrArCrArCrGrGrCrArGrGrGrUrCrArGrUrUrUrUrA | 16479 |
| | P9R16_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArArCrCrCrUrGrArUrArGr | |
| | | UrUrGrArCrCrCrUrGrC*mC*mG*mU | |
| RNACS6081 | TRAC5_ | mG*mC*mU*rGrGrUrArCrArCrGrGrCrArGrGrGrUrCrArGrUrUrUrUrA | 16480 |
| | P8R16_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArArCrCrCrUrGrArUrArGr | |
| | | UrUrGrArCrCrCrUrG*mC*mC*mG | |
| RNACS6082 | TRAC5_ | mG*mC*mU*rGrGrUrArCrArCrGrGrCrArGrGrGrUrCrArGrUrUrUrUrA | 16481 |
| | P13R18_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArGrArArCrCrCrUrGrArUr | |
| | | ArGrUrUrGrArCrCrCrUrGrCrCrGrUrG*mU*mA*mC | |
| RNACS6083 | TRAC5_ | mG*mC*mU*rGrGrUrArCrArCrGrGrCrArGrGrGrUrCrArGrUrUrUrUrA | 16482 |
| | P12R18_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArGrArArCrCrCrUrGrArUr | |
| | | ArGrUrUrGrArCrCrCrUrGrCrCrGrU*mG*mU*mA | |
| RNACS6084 | TRAC5_ | mG*mC*mU*rGrGrUrArCrArCrGrGrCrArGrGrGrUrCrArGrUrUrUrUrA | 16483 |
| | P11R18_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArGrArArCrCrCrUrGrArUr | |
| | | ArGrUrUrGrArCrCrCrUrGrCrCrG*mU*mG*mU | |
| RNACS6085 | TRAC5_ | mG*mC*mU*rGrGrUrArCrArCrGrGrCrArGrGrGrUrCrArGrUrUrUrUrA | 16484 |
| | P10R18_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArGrArArCrCrCrUrGrArUr | |
| | | ArGrUrUrGrArCrCrCrUrGrCrC*mG*mU*mG | |
| RNACS6086 | TRAC5_ | mG*mC*mU*rGrGrUrArCrArCrGrGrCrArGrGrGrUrCrArGrUrUrUrUrA | 16485 |
| | P9R18_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArGrArArCrCrCrUrGrArUr | |
| | | ArGrUrUrGrArCrCrCrUrGrC*mC*mG*mU | |
| RNACS6087 | TRAC5_ | mG*mC*mU*rGrGrUrArCrArCrGrGrCrArGrGrGrUrCrArGrUrUrUrUrA | 16486 |
| | P8R18_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrArGrArArCrCrCrUrGrArUr | |
| | | ArGrUrUrGrArCrCrCrUrG*mC*mC*mG | |
| RNACS6088 | TRAC5_ | mG*mC*mU*rGrGrUrArCrArCrGrGrCrArGrGrGrUrCrArGrUrUrUrUrA | 16487 |
| | P13R20_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrCrCrArGrArArCrCrCrUrGr | |
| | | ArUrArGrUrUrGrArCrCrCrUrGrCrCrGrUrG*mU*mA*mC | |
| RNACS6089 | TRAC5_ | mG*mC*mU*rGrGrUrArCrArCrGrGrCrArGrGrGrUrCrArGrUrUrUrUrA | 16488 |
| | P12R20_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrCrCrArGrArArCrCrCrUrGr | |
| | | AUrArGrUrUrGrArCrCrCrUrGrCrCrGrU*mG*mU*mA | |
| RNACS6090 | TRAC5_ | mG*mC*mU*rGrGrUrArCrArCrGrGrCrArGrGrGrUrCrArGrUrUrUrUrA | 16489 |
| | P11R20_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrCrCrArGrArArCrCrCrUrGr | |
| | | ArUrArGrUrUrGrArCrCrCrUrGrCrCrG*mU*mG*mU | |
| RNACS6091 | TRAC5_ | mG*mC*mU*rGrGrUrArCrArCrGrGrCrArGrGrGrUrCrArGrUrUrUrUrA | 16490 |
| | P10R20_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrCrCrArGrArArCrCrCrUrGr | |
| | | ArUrArGrUrUrGrArCrCrCrUrGrCrC*mG*mU*mG | |
| RNACS6092 | TRAC5_ | mG*mC*mU*rGrGrUrArCrArCrGrGrCrArGrGrGrUrCrArGrUrUrUrUrA | 16491 |
| | P9R20_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrCrCrArGrArArCrCrCrUrGr | |
| | | ArUrArGrUrUrGrArCrCrCrUrGrC*mC*mG*mU | |
| RNACS6093 | TRAC5_ | mG*mC*mU*rGrGrUrArCrArCrGrGrCrArGrGrGrUrCrArGrUrUrUrUrA | 16492 |
| | P8R20_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrCrCrArGrArArCrCrCrUrGr | |
| | | ArUrArGrUrUrGrArCrCrCrUrG*mC*mC*mG | |
| RNACS6094 | TRAC5_ | mG*mC*mU*rGrGrUrArCrArCrGrGrCrArGrGrGrUrCrArGrUrUrUrUrA | 16493 |
| | P13R22_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrArUrCrCrArGrArArCrCrCr | |
| | | UrGrArUrArGrUrUrGrArCrCrCrUrGrCrCrGrUrG*mU*mA*mC | |
| RNACS6095 | TRAC5_ | mG*mC*mU*rGrGrUrArCrArCrGrGrCrArGrGrGrUrCrArGrUrUrUrUrA | 16494 |
| | P12R22_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrArUrCrCrArGrArArCrCrCr | |
| | | UrGrArUrArGrUrUrGrArCrCrCrUrGrCrCrGrU*mG*mU*mA | |
| RNACS6096 | TRAC5_ | mG*mC*mU*rGrGrUrArCrArCrGrGrCrArGrGrGrUrCrArGrUrUrUrUrA | 16495 |
| | P11R22_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrArUrCrCrArGrArArCrCrCr | |
| | | UrGrArUrArGrUrUrGrArCrCrCrUrGrCrCrG*mU*mG*mU | |
| RNACS6097 | TRAC5_ | mG*mC*mU*rGrGrUrArCrArCrGrGrCrArGrGrGrUrCrArGrUrUrUrUrA | 16496 |
| | P10R22_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrArUrCrCrArGrArArCrCrCr | |
| | | UrGrArUrArGrUrUrGrArCrCrCrUrGrCrC*mG*mU*mG | |
| RNACS6098 | TRAC5_ | mG*mC*mU*rGrGrUrArCrArCrGrGrCrArGrGrGrUrCrArGrUrUrUrUrA | 16497 |
| | P9R22_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrArUrCrCrArGrArArCrCrCr | |
| | | UrGrArUrArGrUrUrGrArCrCrCrUrGrC*mC*mG*mU | |
| RNACS6099 | TRAC5_ | mG*mC*mU*rGrGrUrArCrArCrGrGrCrArGrGrGrUrCrArGrUrUrUrUr | 16498 |
| | P8R22_ | ArGrAmGmCmUmAmGmAmAmAmUmAmGmCrArArGrUrUrArArArArUrArAr | |
| | ins7_ | GrGrCrUrArGrUrCrCrGrUrUrArUrCrAmAmCmUmUmGmAmAmAmAmAmGm | |
| | nick | UmGmGmCmAmCmCmGmAmGmUmCmGmGmUmGmCrUrArUrCrCrArGrArArCr | |
| | | CrCrUrGrArUrArGrUrUrGrArCrCrCrUrG*mC*mC*mG | |
| RNACS6100 | TRAC5_ | mG*mC*mU*rGrGrUrArCrArCrGrGrCrArGrGrGrUrCrArGrUrUrUrUrA | 16499 |
| | P13R24_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArUrArUrCrCrArGrArArCr | |
| | | CrCrUrGrArUrArGrUrUrGrArCrCrCrUrGrCrCrGrUrG*mU*mA*mC | |
| RNACS6101 | TRAC5_ | mG*mC*mU*rGrGrUrArCrArCrGrGrCrArGrGrGrUrCrArGrUrUrUrUrA | 16500 |
| | P12R24_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArUrArUrCrCrArGrArArCr | |
| | | CrCrUrGrArUrArGrUrUrGrArCrCrCrUrGrCrCrGrU*mG*mU*mA | |
| RNACS6102 | TRAC5_ | mG*mC*mU*rGrGrUrArCrArCrGrGrCrArGrGrGrUrCrArGrUrUrUrUrA | 16501 |
| | P11R24_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArUrArUrCrCrArGrArArCr | |
| | | CrCrUrGrArUrArGrUrUrGrArCrCrCrUrGrCrCrG*mU*mG*mU | |
| RNACS6103 | TRAC5_ | mG*mC*mU*rGrGrUrArCrArCrGrGrCrArGrGrGrUrCrArGrUrUrUrUrA | 16502 |
| | P10R24_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArUrArUrCrCrArGrArArCr | |
| | | CrCrUrGrArUrArGrUrUrGrArCrCrCrUrGrCrC*mG*mU*mG | |
| RNACS6104 | TRAC5_ | mG*mC*mU*rGrGrUrArCrArCrGrGrCrArGrGrGrUrCrArGrUrUrUrUrA | 16503 |
| | P9R24_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArUrArUrCrCrArGrArArCr | |
| | | CrCrUrGrArUrArGrUrUrGrArCrCrCrUrGrC*mC*mG*mU | |
| RNACS6105 | TRAC5_ | mG*mC*mU*rGrGrUrArCrArCrGrGrCrArGrGrGrUrCrArGrUrUrUrUrA | 16504 |
| | POR24_ | rGrAmGmCmUmAmGmAmAmAmUmAmGmCrArArGrUrUrArArArArUrArArGr | |
| | ins7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrGrArUrArUrCrCrArGrArArCr | |
| | | CrCrUrGrArUrArGrUrUrGrArCrCrCrUrG*mC*mC*mG | |
| RNACS6106 | TRAC5_ | mG*mC*mU*rGrGrUrArCrArCrGrGrCrArGrGrGrUrCrArGrUrUrUrUrA | 16505 |
| | P13R5_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArGrArArCrCrCrCrUrGrCrCr | |
| | | GrUrG*mU*mA*mC | |
| RNACS6107 | TRAC5_ | mG*mC*mU*rGrGrUrArCrArCrGrGrCrArGrGrGrUrCrArGrUrUrUrUrA | 16506 |
| | P12R5_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArGrArArCrCrCrCrUrGrCrCr | |
| | | GrU*mG*mU*mA | |
| RNACS6108 | TRAC5_ | mG*mC*mU*rGrGrUrArCrArCrGrGrCrArGrGrGrUrCrArGrUrUrUrUrA | 16507 |
| | P11R5_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArGrArArCrCrCrCrUrGrCrCr | |
| | | G*mU*mG*mU | |
| RNACS6109 | TRAC5_ | mG*mC*mU*rGrGrUrArCrArCrGrGrCrArGrGrGrUrCrArGrUrUrUrUrA | 16508 |
| | P10R5_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArGrArArCrCrCrCrUrGrCrC* | |
| | | mG*mU*mG | |
| RNACS6110 | TRAC5_ | mG*mC*mU*rGrGrUrArCrArCrGrGrCrArGrGrGrUrCrArGrUrUrUrUrA | 16509 |
| | P9R5_del5_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrArGrArArCrCrCrCrUrGrC*mC | |
| | | *mG*mU | |
| RNACS6111 | TRAC5_ | mG*mC*mU*rGrGrUrArCrArCrGrGrCrArGrGrGrUrCrArGrUrUrUrUrA | 16510 |
| | P8R5_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArGrArArCrCrCrCrUrG*mC*m | |
| | | C*mG | |
| RNACS6112 | TRAC5_ | mG*mC*mU*rGrGrUrArCrArCrGrGrCrArGrGrGrUrCrArGrUrUrUrUrA | 16511 |
| | P13R7_del5_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrCrArGrArArCrCrCrCrUrGr | |
| | | CrCrGrUrG*mU*mA*mC | |
| RNACS6113 | TRAC5_ | mG*mC*mU*rGrGrUrArCrArCrGrGrCrArGrGrGrUrCrArGrUrUrUrUrA | 16512 |
| | P12R7_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrArGrArArCrCrCrCrUrGr | |
| | | CrCrGrU*mG*mU*mA | |
| RNACS6114 | TRAC5_ | mG*mC*mU*rGrGrUrArCrArCrGrGrCrArGrGrGrUrCrArGrUrUrUrUrA | 16513 |
| | P11R7_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrArGrArArCrCrCrCrUrGr | |
| | | CrCrG*mU*mG*mU | |
| RNACS6115 | TRAC5_ | mG*mC*mU*rGrGrUrArCrArCrGrGrCrArGrGrGrUrCrArGrUrUrUrUrA | 16514 |
| | P10R7_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrArGrArArCrCrCrCrUrGr | |
| | | CrC*mG*mU*mG | |
| RNACS6116 | TRAC5_ | mG*mC*mU*rGrGrUrArCrArCrGrGrCrArGrGrGrUrCrArGrUrUrUrUrA | 16515 |
| | P9R7_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrArGrArArCrCrCrCrUrGr | |
| | | C*mC*mG*mU | |
| RNACS6117 | TRAC5_ | mG*mC*mU*rGrGrUrArCrArCrGrGrCrArGrGrGrUrCrArGrUrUrUrUrA | 16516 |
| | P8R7_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrArGrArArCrCrCrCrUrG* | |
| | | mC*mC*mG | |
| RNACS6118 | TRAC5_ | mG*mC*mU*rGrGrUrArCrArCrGrGrCrArGrGrGrUrCrArGrUrUrUrUrA | 16517 |
| | P13R9_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArUrCrCrArGrArArCrCrCrCr | |
| | | UrGrCrCrGrUrG*mU*mA*mC | |
| RNACS6119 | TRAC5_ | mG*mC*mU*rGrGrUrArCrArCrGrGrCrArGrGrGrUrCrArGrUrUrUrUrA | 16518 |
| | P12R9_del5_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrArUrCrCrArGrArArCrCrCrCr | |
| | | UrGrCrCrGrU*mG*mU*mA | |
| RNACS6120 | TRAC5_ | mG*mC*mU*rGrGrUrArCrArCrGrGrCrArGrGrGrUrCrArGrUrUrUrUrA | 16519 |
| | P11R9_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArUrCrCrArGrArArCrCrCrCr | |
| | | UrGrCrCrG*mU*mG*mU | |
| RNACS6121 | TRAC5_ | mG*mC*mU*rGrGrUrArCrArCrGrGrCrArGrGrGrUrCrArGrUrUrUrUrA | 16520 |
| | P10R9_ | rGrAmGmCmUmAmGmAmAmAmUmAmGmCrArArGrUrUrArArArArUrArArGr | |
| | de15_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArUrCrCrArGrArArCrCrCrCr | |
| | | UrGrCrC*mG*mU*mG | |
| RNACS6122 | TRAC5_ | mG*mC*mU*rGrGrUrArCrArCrGrGrCrArGrGrGrUrCrArGrUrUrUrUrA | 16521 |
| | P9R9_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArUrCrCrArGrArArCrCrCrCr | |
| | | UrGrC*mC*mG*mU | |
| RNACS6123 | TRAC5_ | mG*mC*mU*rGrGrUrArCrArCrGrGrCrArGrGrGrUrCrArGrUrUrUrUrA | 16522 |
| | P8R9_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArUrCrCrArGrArArCrCrCrCr | |
| | | UrG*mC*mC*mG | |
| RNACS6124 | TRAC5_ | mG*mC*mU*rGrGrUrArCrArCrGrGrCrArGrGrGrUrCrArGrUrUrUrUrA | 16523 |
| | P13R11_del5_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrArUrArUrCrCrArGrArArCrCr | |
| | | CrCrUrGrCrCrGrUrG*mU*mA*mC | |
| RNACS6125 | TRAC5_ | mG*mC*mU*rGrGrUrArCrArCrGrGrCrArGrGrGrUrCrArGrUrUrUrUrA | 16524 |
| | P12R11_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArUrArUrCrCrArGrArArCrCr | |
| | | CrCrUrGrCrCrGrU*mG*mU*mA | |
| RNACS6126 | TRAC5_ | mG*mC*mU*rGrGrUrArCrArCrGrGrCrArGrGrGrUrCrArGrUrUrUrUrA | 16525 |
| | P11R11_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArUrArUrCrCrArGrArArCrCr | |
| | | CrCrUrGrCrCrG*mU*mG*mU | |
| RNACS6127 | TRAC5_ | mG*mC*mU*rGrGrUrArCrArCrGrGrCrArGrGrGrUrCrArGrUrUrUrUrA | 16526 |
| | P10R11_del5_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrArUrArUrCrCrArGrArArCrCr | |
| | | CrCrUrGrCrC*mG*mU*mG | |
| RNACS6128 | TRAC5_ | mG*mC*mU*rGrGrUrArCrArCrGrGrCrArGrGrGrUrCrArGrUrUrUrUrA | 16527 |
| | P9R11_ | rGrAmGmCmUmAmGmAmAmAmUmAmGmCrArArGrUrUrArArArArUrArArGr | |
| | de15_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArUrArUrCrCrArGrArArCrCr | |
| | | CrCrUrGrC*mC*mG*mU | |
| RNACS6129 | TRAC5_ | mG*mC*mU*rGrGrUrArCrArCrGrGrCrArGrGrGrUrCrArGrUrUrUrUrA | 16528 |
| | P8R11_de15_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrArUrArUrCrCrArGrArArCrCr | |
| | | CrCrUrG*mC*mC*mG | |
| RNACS6130 | TRAC5_ | mG*mC*mU*rGrGrUrArCrArCrGrGrCrArGrGrGrUrCrArGrUrUrUrUrA | 16529 |
| | P13R13_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArGrArUrArUrCrCrArGrArAr | |
| | | CrCrCrCrUrGrCrCrGrUrG*mU*mA*mC | |
| RNACS6131 | TRAC5_ | mG*mC*mU*rGrGrUrArCrArCrGrGrCrArGrGrGrUrCrArGrUrUrUrUrA | 16530 |
| | P12R13_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArGrArUrArUrCrCrArGrArAr | |
| | | CrCrCrCrUrGrCrCrGrU*mG*mU*mA | |
| RNACS6132 | TRAC5_ | mG*mC*mU*rGrGrUrArCrArCrGrGrCrArGrGrGrUrCrArGrUrUrUrUrA | 16531 |
| | P11R13_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArGrArUrArUrCrCrArGrArAr | |
| | | CrCrCrCrUrGrCrCrG*mU*mG*mU | |
| RNACS6133 | TRAC5_ | mG*mC*mU*rGrGrUrArCrArCrGrGrCrArGrGrGrUrCrArGrUrUrUrUrA | 16532 |
| | P10R13_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArGrArUrArUrCrCrArGrArAr | |
| | | CrCrCrCrUrGrCrC*mG*mU*mG | |
| RNACS6134 | TRAC5_ | mG*mC*mU*rGrGrUrArCrArCrGrGrCrArGrGrGrUrCrArGrUrUrUrUrA | 16533 |
| | P9R13_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArGrArUrArUrCrCrArGrArAr | |
| | | CrCrCrCrUrGrC*mC*mG*mU | |
| RNACS6135 | TRAC5_ | mG*mC*mU*rGrGrUrArCrArCrGrGrCrArGrGrGrUrCrArGrUrUrUrUrA | 16534 |
| | P8R13_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArGrArUrArUrCrCrArGrArAr | |
| | | CrCrCrCrUrG*mC*mC*mG | |
| RNACS6136 | TRAC5_ | mG*mC*mU*rGrGrUrArCrArCrGrGrCrArGrGrGrUrCrArGrUrUrUrUrA | 16535 |
| | P13R15_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArCrArGrArUrArUrCrCrArGr | |
| | | ArArCrCrCrCrUrGrCrCrGrUrG*mU*mA*mC | |
| RNACS6137 | TRAC5_ | mG*mC*mU*rGrGrUrArCrArCrGrGrCrArGrGrGrUrCrArGrUrUrUrUrA | 16536 |
| | P12R15_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArCrArGrArUrArUrCrCrArGr | |
| | | ArArCrCrCrCrUrGrCrCrGrU*mG*mU*mA | |
| RNACS6138 | TRAC5_ | mG*mC*mU*rGrGrUrArCrArCrGrGrCrArGrGrGrUrCrArGrUrUrUrUrA | 16537 |
| | P11R15_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArCrArGrArUrArUrCrCrArGr | |
| | | ArArCrCrCrCrUrGrCrCrG*mU*mG*mU | |
| RNACS6139 | TRAC5_ | mG*mC*mU*rGrGrUrArCrArCrGrGrCrArGrGrGrUrCrArGrUrUrUrUrA | 16538 |
| | P10R15_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArCrArGrArUrArUrCrCrArGr | |
| | | ArArCrCrCrCrUrGrCrC*mG*mU*mG | |
| RNACS6140 | TRAC5_ | mG*mC*mU*rGrGrUrArCrArCrGrGrCrArGrGrGrUrCrArGrUrUrUrUrA | 16539 |
| | P9R15_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArCrArGrArUrArUrCrCrArGr | |
| | | ArArCrCrCrCrUrGrC*mC*mG*mU | |
| RNACS6141 | TRAC5_ | mG*mC*mU*rGrGrUrArCrArCrGrGrCrArGrGrGrUrCrArGrUrUrUrUrA | 16540 |
| | P8R15_ | rGrAmGmCmUmAmGmAmAmAmUmAmGmCrArArGrUrUrArArArArUrArArGr | |
| | de15_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArCrArGrArUrArUrCrCrArGr | |
| | | ArArCrCrCrCrUrG*mC*mC*mG | |
| RNACS6142 | TRAC5_ | mG*mC*mU*rGrGrUrArCrArCrGrGrCrArGrGrGrUrCrArGrUrUrUrUrA | 16541 |
| | P13R17_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrArCrArGrArUrArUrCrCr | |
| | | ArGrArArCrCrCrCrUrGrCrCrGrUrG*mU*mA*mC | |
| RNACS6143 | TRAC5_ | mG*mC*mU*rGrGrUrArCrArCrGrGrCrArGrGrGrUrCrArGrUrUrUrUrA | 16542 |
| | P12R17_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrArCrArGrArUrArUrCrCr | |
| | | ArGrArArCrCrCrCrUrGrCrCrGrU*mG*mU*mA | |
| RNACS6144 | TRAC5_ | mG*mC*mU*rGrGrUrArCrArCrGrGrCrArGrGrGrUrCrArGrUrUrUrUrA | 16543 |
| | P11R17_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrArCrArGrArUrArUrCrCr | |
| | | ArGrArArCrCrCrCrUrGrCrCrG*mU*mG*mU | |
| RNACS6145 | TRAC5_ | mG*mC*mU*rGrGrUrArCrArCrGrGrCrArGrGrGrUrCrArGrUrUrUrUrA | 16544 |
| | P10R17_del5_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrCrArCrArGrArUrArUrCrCr | |
| | | ArGrArArCrCrCrCrUrGrCrC*mG*mU*mG | |
| RNACS6146 | TRAC5_ | mG*mC*mU*rGrGrUrArCrArCrGrGrCrArGrGrGrUrCrArGrUrUrUrUrA | 16545 |
| | P9R17_ | rGrAmGmCmUmAmGmAmAmAmUmAmGmCrArArGrUrUrArArArArUrArArGr | |
| | de15_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrArCrArGrArUrArUrCrCr | |
| | | ArGrArArCrCrCrCrUrGrC*mC*mG*mU | |
| RNACS6147 | TRAC5_ | mG*mC*mU*rGrGrUrArCrArCrGrGrCrArGrGrGrUrCrArGrUrUrUrUrA | 16546 |
| | P8R17_ | rGrAmGmCmUmAmGmAmAmAmUmAmGmCrArArGrUrUrArArArArUrArArGr | |
| | del5_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrArCrArGrArUrArUrCrCr | |
| | | ArGrArArCrCrCrCrUrG*mC*mC*mG | |
| RNACS6148 | TRAC5_ | mG*mC*mU*rGrGrUrArCrArCrGrGrCrArGrGrGrUrCrArGrUrUrUrUrA | 16547 |
| | P13R5_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrArGrArCrCrCrUrGrCrCr | |
| | | GrUrG*mU*mA*mC | |
| RNACS6149 | TRAC5_ | mG*mC*mU*rGrGrUrArCrArCrGrGrCrArGrGrGrUrCrArGrUrUrUrUrA | 16548 |
| | P12R5_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrArGrArCrCrCrUrGrCrCr | |
| | | GrU*mG*mU*mA | |
| RNACS6150 | TRAC5_ | mG*mC*mU*rGrGrUrArCrArCrGrGrCrArGrGrGrUrCrArGrUrUrUrUrA | 16549 |
| | P11R5_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrArGrArCrCrCrUrGrCrCr | |
| | | G*mU*mG*mU | |
| RNACS6151 | TRAC5_ | mG*mC*mU*rGrGrUrArCrArCrGrGrCrArGrGrGrUrCrArGrUrUrUrUrA | 16550 |
| | P10R5_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrArGrArCrCrCrUrGrCrC* | |
| | | mG*mU*mG | |
| RNACS6152 | TRAC5_ | mG*mC*mU*rGrGrUrArCrArCrGrGrCrArGrGrGrUrCrArGrUrUrUrUrA | 16551 |
| | P9R5_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrArGrArCrCrCrUrGrC*mC | |
| | | *mG*mU | |
| RNACS6153 | TRAC5_ | mG*mC*mU*rGrGrUrArCrArCrGrGrCrArGrGrGrUrCrArGrUrUrUrUrA | 16552 |
| | P8R5_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrArGrArCrCrCrUrG*mC*m | |
| | | C*mG | |
| RNACS6154 | TRAC5_ | mG*mC*mU*rGrGrUrArCrArCrGrGrCrArGrGrGrUrCrArGrUrUrUrUrA | 16553 |
| | P13R7_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArUrCrCrArGrArCrCrCrUrGr | |
| | | CrCrGrUrG*mU*mA*mC | |
| RNACS6155 | TRAC5_ | mG*mC*mU*rGrGrUrArCrArCrGrGrCrArGrGrGrUrCrArGrUrUrUrUrA | 16554 |
| | P12R7_del7_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrArUrCrCrArGrArCrCrCrUrGr | |
| | | CrCrGrU*mG*mU*mA | |
| RNACS6156 | TRAC5_ | mG*mC*mU*rGrGrUrArCrArCrGrGrCrArGrGrGrUrCrArGrUrUrUrUrA | 16555 |
| | P11R7_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArUrCrCrArGrArCrCrCrUrGr | |
| | | CrCrG*mU*mG*mU | |
| RNACS6157 | TRAC5_ | mG*mC*mU*rGrGrUrArCrArCrGrGrCrArGrGrGrUrCrArGrUrUrUrUrA | 16556 |
| | P10R7_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArUrCrCrArGrArCrCrCrUrGr | |
| | | CrC*mG*mU*mG | |
| RNACS6158 | TRAC5_ | mG*mC*mU*rGrGrUrArCrArCrGrGrCrArGrGrGrUrCrArGrUrUrUrUrA | 16557 |
| | P9R7_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArUrCrCrArGrArCrCrCrUrGr | |
| | | C*mC*mG*mU | |
| RNACS6159 | TRAC5_ | mG*mC*mU*rGrGrUrArCrArCrGrGrCrArGrGrGrUrCrArGrUrUrUrUrA | 16558 |
| | P8R7_del7_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrArUrCrCrArGrArCrCrCrUrG* | |
| | | mC*mC*mG | |
| RNACS6160 | TRAC5_ | mG*mC*mU*rGrGrUrArCrArCrGrGrCrArGrGrGrUrCrArGrUrUrUrUrA | 16559 |
| | P13R9_del7_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrArUrArUrCrCrArGrArCrCrCr | |
| | | UrGrCrCrGrUrG*mU*mA*mC | |
| RNACS6161 | TRAC5_ | mG*mC*mU*rGrGrUrArCrArCrGrGrCrArGrGrGrUrCrArGrUrUrUrUrA | 16560 |
| | P12R9_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArUrArUrCrCrArGrArCrCrCr | |
| | | UrGrCrCrGrU*mG*mU*mA | |
| RNACS6162 | TRAC5_ | mG*mC*mU*rGrGrUrArCrArCrGrGrCrArGrGrGrUrCrArGrUrUrUrUrA | 16561 |
| | P11R9_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArUrArUrCrCrArGrArCrCrCr | |
| | | UrGrCrCrG*mU*mG*mU | |
| RNACS6163 | TRAC5_ | mG*mC*mU*rGrGrUrArCrArCrGrGrCrArGrGrGrUrCrArGrUrUrUrUrA | 16562 |
| | P10R9_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArUrArUrCrCrArGrArCrCrCr | |
| | | UrGrCrC*mG*mU*mG | |
| RNACS6164 | TRAC5_ | mG*mC*mU*rGrGrUrArCrArCrGrGrCrArGrGrGrUrCrArGrUrUrUrUrA | 16563 |
| | P9R9_del7_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrArUrArUrCrCrArGrArCrCrCr | |
| | | UrGrC*mC*mG*mU | |
| RNACS6165 | TRAC5_ | mG*mC*mU*rGrGrUrArCrArCrGrGrCrArGrGrGrUrCrArGrUrUrUrUrA | 16564 |
| | P8R9_del7_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrArUrArUrCrCrArGrArCrCrCr | |
| | | UrG*mC*mC*mG | |
| RNACS6166 | TRAC5_ | mG*mC*mU*rGrGrUrArCrArCrGrGrCrArGrGrGrUrCrArGrUrUrUrUrA | 16565 |
| | P13R11_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArGrArUrArUrCrCrArGrArCr | |
| | | CrCrUrGrCrCrGrUrG*mU*mA*mC | |
| RNACS6167 | TRAC5_ | mG*mC*mU*rGrGrUrArCrArCrGrGrCrArGrGrGrUrCrArGrUrUrUrUrA | 16566 |
| | P12R11_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArGrArUrArUrCrCrArGrArCr | |
| | | CrCrUrGrCrCrGrU*mG*mU*mA | |
| RNACS6168 | TRAC5_ | mG*mC*mU*rGrGrUrArCrArCrGrGrCrArGrGrGrUrCrArGrUrUrUrUrA | 16567 |
| | P11R11_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArGrArUrArUrCrCrArGrArCr | |
| | | CrCrUrGrCrCrG*mU*mG*mU | |
| RNACS6169 | TRAC5_ | mG*mC*mU*rGrGrUrArCrArCrGrGrCrArGrGrGrUrCrArGrUrUrUrUrA | 16568 |
| | P10R11_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArGrArUrArUrCrCrArGrArCr | |
| | | CrCrUrGrCrC*mG*mU*mG | |
| RNACS6170 | TRAC5_ | mG*mC*mU*rGrGrUrArCrArCrGrGrCrArGrGrGrUrCrArGrUrUrUrUrA | 16569 |
| | P9R11_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArGrArUrArUrCrCrArGrArCr | |
| | | CrCrUrGrC*mC*mG*mU | |
| RNACS6171 | TRAC5_ | mG*mC*mU*rGrGrUrArCrArCrGrGrCrArGrGrGrUrCrArGrUrUrUrUrA | 16570 |
| | P8R11_del7_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrArGrArUrArUrCrCrArGrArCr | |
| | | CrCrUrG*mC*mC*mG | |
| RNACS6172 | TRAC5_ | mG*mC*mU*rGrGrUrArCrArCrGrGrCrArGrGrGrUrCrArGrUrUrUrUrA | 16571 |
| | P13R13_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArCrArGrArUrArUrCrCrArGr | |
| | | ArCrCrCrUrGrCrCrGrUrG*mU*mA*mC | |
| RNACS6173 | TRAC5_ | mG*mC*mU*rGrGrUrArCrArCrGrGrCrArGrGrGrUrCrArGrUrUrUrUrA | 16572 |
| | P12R13_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArCrArGrArUrArUrCrCrArGr | |
| | | ArCrCrCrUrGrCrCrGrU*mG*mU*mA | |
| RNACS6174 | TRAC5_ | mG*mC*mU*rGrGrUrArCrArCrGrGrCrArGrGrGrUrCrArGrUrUrUrUrA | 16573 |
| | P11R13_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArCrArGrArUrArUrCrCrArGr | |
| | | ArCrCrCrUrGrCrCrG*mU*mG*mU | |
| RNACS6175 | TRAC5_ | mG*mC*mU*rGrGrUrArCrArCrGrGrCrArGrGrGrUrCrArGrUrUrUrUrA | 16574 |
| | P10R13_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArCrArGrArUrArUrCrCrArGr | |
| | | ArCrCrCrUrGrCrC*mG*mU*mG | |
| RNACS6176 | TRAC5_ | mG*mC*mU*rGrGrUrArCrArCrGrGrCrArGrGrGrUrCrArGrUrUrUrUrA | 16575 |
| | P9R13_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArCrArGrArUrArUrCrCrArGr | |
| | | ArCrCrCrUrGrC*mC*mG*mU | |
| RNACS6177 | TRAC5_ | mG*mC*mU*rGrGrUrArCrArCrGrGrCrArGrGrGrUrCrArGrUrUrUrUrA | 16576 |
| | P8R13_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrArCrArGrArUrArUrCrCrArGr | |
| | | ArCrCrCrUrG*mC*mC*mG | |
| RNACS6178 | TRAC5_ | mG*mC*mU*rGrGrUrArCrArCrGrGrCrArGrGrGrUrCrArGrUrUrUrUrA | 16577 |
| | P13R15_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrArCrArGrArUrArUrCrCr | |
| | | ArGrArCrCrCrUrGrCrCrGrUrG*mU*mA*mC | |
| RNACS6179 | TRAC5_ | mG*mC*mU*rGrGrUrArCrArCrGrGrCrArGrGrGrUrCrArGrUrUrUrUrA | 16578 |
| | P12R15_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrArCrArGrArUrArUrCrCr | |
| | | ArGrArCrCrCrUrGrCrCrGrU*mG*mU*mA | |
| RNACS6180 | TRAC5_ | mG*mC*mU*rGrGrUrArCrArCrGrGrCrArGrGrGrUrCrArGrUrUrUrUrA | 16579 |
| | P11R15_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrArCrArGrArUrArUrCrCr | |
| | | ArGrArCrCrCrUrGrCrCrG*mUmG*mU | |
| RNACS6181 | TRAC5_ | mG*mC*mU*rGrGrUrArCrArCrGrGrCrArGrGrGrUrCrArGrUrUrUrUrA | 16580 |
| | P10R15_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrArCrArGrArUrArUrCrCr | |
| | | ArGrArCrCrCrUrGrCrC*mG*mU*mG | |
| RNACS6182 | TRAC5_ | mG*mC*mU*rGrGrUrArCrArCrGrGrCrArGrGrGrUrCrArGrUrUrUrUrA | 16581 |
| | P9R15_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrArCrArGrArUrArUrCrCr | |
| | | ArGrArCrCrCrUrGrC*mC*mG*mU | |
| RNACS6183 | TRAC5_ | mG*mC*mU*rGrGrUrArCrArCrGrGrCrArGrGrGrUrCrArGrUrUrUrUrA | 16582 |
| | P8R15_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrCrCrArCrArGrArUrArUrCrCr | |
| | | ArGrArCrCrCrUrG*mC*mC*mG | |
| RNACS6184 | TRAC5_ | mG*mC*mU*rGrGrUrArCrArCrGrGrCrArGrGrGrUrCrArGrUrUrUrUrA | 16583 |
| | P13R17_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrCrCrCrArCrArGrArUrArUr | |
| | | CrCrArGrArCrCrCrUrGrCrCrGrUrG*mU*mA*mC | |
| RNACS6185 | TRAC5_ | mG*mC*mU*rGrGrUrArCrArCrGrGrCrArGrGrGrUrCrArGrUrUrUrUrA | 16584 |
| | P12R17_ | rGrAmGmCmUmAmGmAmAmAmUmAmGmCrArArGrUrUrArArArArUrArArGr | |
| | del7_ | GrCrUrArGrUrCrCrGrUrUrArUrCrAmAmCmUmUmGmAmAmAmAmAmGmUmG | |
| | nick | mGmCmAmCmCmGmAmGmUmCmGmGmUmGmCrUrCrCrCrArCrArGrArUrArUr | |
| | | CrCrArGrArCrCrCrUrGrCrCrGrU*mG*mU*mA | |
| RNACS6186 | TRAC5_ | mG*mC*mU*rGrGrUrArCrArCrGrGrCrArGrGrGrUrCrArGrUrUrUrUrA | 16585 |
| | P11R17_del7_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrUrCrCrCrArCrArGrArUrArUr | |
| | | CrCrArGrArCrCrCrUrGrCrCrG*mU*mG*mU | |
| RNACS6187 | TRAC5_ | mG*mC*mU*rGrGrUrArCrArCrGrGrCrArGrGrGrUrCrArGrUrUrUrUrA | 16586 |
| | P10R17_del7_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrUrCrCrCrArCrArGrArUrArUr | |
| | | CrCrArGrArCrCrCrUrGrCrC*mG*mU*mG | |
| RNACS6188 | TRAC5_ | mG*mC*mU*rGrGrUrArCrArCrGrGrCrArGrGrGrUrCrArGrUrUrUrUrA | 16587 |
| | P9R17_del7_ | rGrAmGmCmUmAmGmAmAmAmUmAmGmCrArArGrUrUrArArArArUrArArGr | |
| | nick | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrUrCrCrCrArCrArGrArUrArUr | |
| | | CrCrArGrArCrCrCrUrGrC*mC*mG*mU | |
| RNACS6189 | TRAC5_ | UmAmGmCrArArGrUrUrArArArArUrArArGrGrCrUrArGrUrCrCrGrUrU | 16588 |
| | P8R17_del7_ | rArUrCrAmAmCmUmUmGmAmAmAmAmAmGmUmGmGmCmAmCmCmGmAmGmUmCm | |
| | nick | GmGmUmGmCrUrCrCrCrArCrArGrArUrArUrCrCrArGrArCrCrCrUrG | |
| | | *mC*mC*mGmG*mC*mU*rGrGrUrArCrArCrGrGrCrArGrGrGrUrCrArGr | |
| | | UrUrUrUrArGrAmGmCmUmAmGmAmAmAm | |
| RNACS6190 | TRAC5_ | mG*mC*mU*rGrGrUrArCrArCrGrGrCrArGrGrGrUrCrArGrUrUrUrUrA | 16589 |
| | P13R13_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArArCrCrCrUrArGrUrGrUrGr | |
| | | ArCrCrCrUrGrCrCrGrUrG*mU*mA*mC | |
| RNACS6191 | TRAC5_ | mG*mC*mU*rGrGrUrArCrArCrGrGrCrArGrGrGrUrCrArGrUrUrUrUrA | 16590 |
| | P12R13_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArArCrCrCrUrArGrUrGrUrGr | |
| | | ArCrCrCrUrGrCrCrGrU*mG*mU*mA | |
| RNACS6192 | TRAC5_ | mG*mC*mU*rGrGrUrArCrArCrGrGrCrArGrGrGrUrCrArGrUrUrUrUrA | 16591 |
| | P11R13_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArArCrCrCrUrArGrUrGrUrGr | |
| | | ArCrCrCrUrGrCrCrG*mU*mG*mU | |
| RNACS6193 | TRAC5_ | mG*mC*mU*rGrGrUrArCrArCrGrGrCrArGrGrGrUrCrArGrUrUrUrUrA | 16592 |
| | P10R13_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArArCrCrCrUrArGrUrGrUrGr | |
| | | ArCrCrCrUrGrCrC*mG*mU*mG | |
| RNACS6194 | TRAC5_ | mG*mC*mU*rGrGrUrArCrArCrGrGrCrArGrGrGrUrCrArGrUrUrUrUrA | 16593 |
| | P9R13_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArArCrCrCrUrArGrUrGrUrGr | |
| | | ArCrCrCrUrGrC*mC*mG*mU | |
| RNACS6195 | TRAC5_ | mG*mC*mU*rGrGrUrArCrArCrGrGrCrArGrGrGrUrCrArGrUrUrUrUrA | 16594 |
| | P8R13_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArArCrCrCrUrArGrUrGrUrGr | |
| | | ArCrCrCrUrG*mC*mC*mG | |
| RNACS6196 | TRAC5_ | mG*mC*mU*rGrGrUrArCrArCrGrGrCrArGrGrGrUrCrArGrUrUrUrUrA | 16595 |
| | P13R15_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrArArCrCrCrUrArGrUrGr | |
| | | UrGrArCrCrCrUrGrCrCrGrUrG*mU*mA*mC | |
| RNACS6197 | TRAC5_ | mG*mC*mU*rGrGrUrArCrArCrGrGrCrArGrGrGrUrCrArGrUrUrUrUrA | 16596 |
| | P12R15_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrArArCrCrCrUrArGrUrGr | |
| | | UGrArCrCrCrUrGrCrCrGrU*mG*mU*mA | |
| RNACS6198 | TRAC5_ | mG*mC*mU*rGrGrUrArCrArCrGrGrCrArGrGrGrUrCrArGrUrUrUrUrA | 16597 |
| | P11R15_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrArArCrCrCrUrArGrUrGr | |
| | | UrGrArCrCrCrUrGrCrCrG*mU*mG*mU | |
| RNACS6199 | TRAC5_ | mG*mC*mU*rGrGrUrArCrArCrGrGrCrArGrGrGrUrCrArGrUrUrUrUrA | 16598 |
| | P10R15_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrArArCrCrCrUrArGrUrGr | |
| | | UrGrArCrCrCrUrGrCrC*mG*mU*mG | |
| RNACS6200 | TRAC5_ | mG*mC*mU*rGrGrUrArCrArCrGrGrCrArGrGrGrUrCrArGrUrUrUrUrA | 16599 |
| | P9R15_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrArArCrCrCrUrArGrUrGr | |
| | | UrGrArCrCrCrUrGrC*mC*mG*mU | |
| RNACS6201 | TRAC5_ | mG*mC*mU*rGrGrUrArCrArCrGrGrCrArGrGrGrUrCrArGrUrUrUrUrA | 16600 |
| | P8R15_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrArArCrCrCrUrArGrUrGr | |
| | | UrGrArCrCrCrUrG*mC*mC*mG | |
| RNACS6202 | TRAC5_ | mG*mC*mU*rGrGrUrArCrArCrGrGrCrArGrGrGrUrCrArGrUrUrUrUrA | 16601 |
| | P13R17_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrArGrArArCrCrCrUrArGr | |
| | | UrGrUrGrArCrCrCrUrGrCrCrGrUrG*mU*mA*mC | |
| RNACS6203 | TRAC5_ | mG*mC*mU*rGrGrUrArCrArCrGrGrCrArGrGrGrUrCrArGrUrUrUrUrA | 16602 |
| | P12R17_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrArGrArArCrCrCrUrArGr | |
| | | UrGrUrGrArCrCrCrUrGrCrCrGrU*mG*mU*mA | |
| RNACS6204 | TRAC5_ | mG*mC*mU*rGrGrUrArCrArCrGrGrCrArGrGrGrUrCrArGrUrUrUrUrA | 16603 |
| | P11R17_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrArGrArArCrCrCrUrArGr | |
| | | UrGrUrGrArCrCrCrUrGrCrCrG*mU*mG*mU | |
| RNACS6205 | TRAC5_ | mG*mC*mU*rGrGrUrArCrArCrGrGrCrArGrGrGrUrCrArGrUrUrUrUrA | 16604 |
| | P10R17_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrArGrArArCrCrCrUrArGr | |
| | | UrGrUrGrArCrCrCrUrGrCrC*mG*mU*mG | |
| RNACS6206 | TRAC5_ | mG*mC*mU*rGrGrUrArCrArCrGrGrCrArGrGrGrUrCrArGrUrUrUrUrA | 16605 |
| | P9R17_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrArGrArArCrCrCrUrArGr | |
| | | UrGrUrGrArCrCrCrUrGrC*mC*mG*mU | |
| RNACS6207 | TRAC5_ | mG*mC*mU*rGrGrUrArCrArCrGrGrCrArGrGrGrUrCrArGrUrUrUrUrA | 16606 |
| | P8R17_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrArGrArArCrCrCrUrArGr | |
| | | UrGrUrGrArCrCrCrUrG*mC*mC*mG | |
| RNACS6208 | TRAC5_ | mG*mC*mU*rGrGrUrArCrArCrGrGrCrArGrGrGrUrCrArGrUrUrUrUrA | 16607 |
| | P13R19_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrCrCrArGrArArCrCrCrUr | |
| | | ArGrUrGrUrGrArCrCrCrUrGrCrCrGrUrG*mU*mA*mC | |
| RNACS6209 | TRAC5_ | mG*mC*mU*rGrGrUrArCrArCrGrGrCrArGrGrGrUrCrArGrUrUrUrUrA | 16608 |
| | P12R19_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrCrCrArGrArArCrCrCrUr | |
| | | ArGrUrGrUrGrArCrCrCrUrGrCrCrGrU*mG*mU*mA | |
| RNACS6210 | TRAC5_ | mG*mC*mU*rGrGrUrArCrArCrGrGrCrArGrGrGrUrCrArGrUrUrUrUrA | 16609 |
| | P11R19_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrCrCrArGrArArCrCrCrUr | |
| | | ArGrUrGrUrGrArCrCrCrUrGrCrCrG*mU*mG*mU | |
| RNACS6211 | TRAC5_ | mG*mC*mU*rGrGrUrArCrArCrGrGrCrArGrGrGrUrCrArGrUrUrUrUrA | 16610 |
| | P10R19_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrCrCrArGrArArCrCrCrUr | |
| | | ArGrUrGrUrGrArCrCrCrUrGrCrC*mG*mU*mG | |
| RNACS6212 | TRAC5_ | mG*mC*mU*rGrGrUrArCrArCrGrGrCrArGrGrGrUrCrArGrUrUrUrUrA | 16611 |
| | P9R19_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrCrCrArGrArArCrCrCrUr | |
| | | ArGrUrGrUrGrArCrCrCrUrGrC*mC*mG*mU | |
| RNACS6213 | TRAC5_ | mG*mC*mU*rGrGrUrArCrArCrGrGrCrArGrGrGrUrCrArGrUrUrUrUrA | 16612 |
| | P8R19_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrCrCrArGrArArCrCrCrUr | |
| | | ArGrUrGrUrGrArCrCrCrUrG*mC*mC*mG | |
| RNACS6214 | TRAC5_ | mG*mC*mU*rGrGrUrArCrArCrGrGrCrArGrGrGrUrCrArGrUrUrUrUrA | 16613 |
| | P13R21_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrArUrCrCrArGrArArCrCr | |
| | | CrUrArGrUrGrUrGrArCrCrCrUrGrCrCrGrUrG*mU*mA*mC | |
| RNACS6215 | TRAC5_ | mG*mC*mU*rGrGrUrArCrArCrGrGrCrArGrGrGrUrCrArGrUrUrUrUrA | 16614 |
| | P12R21_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrArUrCrCrArGrArArCrCr | |
| | | CrUrArGrUrGrUrGrArCrCrCrUrGrCrCrGrU*mG*mU*mA | |
| RNACS6216 | TRAC5_ | mG*mC*mU*rGrGrUrArCrArCrGrGrCrArGrGrGrUrCrArGrUrUrUrUrA | 16615 |
| | P11R21_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrArUrCrCrArGrArArCrCr | |
| | | CrUrArGrUrGrUrGrArCrCrCrUrGrCrCrG*mU*mG*mU | |
| RNACS6217 | TRAC5_ | mG*mC*mU*rGrGrUrArCrArCrGrGrCrArGrGrGrUrCrArGrUrUrUrUrA | 16616 |
| | P10R21_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrArUrCrCrArGrArArCrCr | |
| | | CrUrArGrUrGrUrGrArCrCrCrUrGrCrC*mG*mU*mG | |
| RNACS6218 | TRAC5_ | mG*mC*mU*rGrGrUrArCrArCrGrGrCrArGrGrGrUrCrArGrUrUrUrUrA | 16617 |
| | P9R21_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrArUrCrCrArGrArArCrCr | |
| | | CrUrArGrUrGrUrGrArCrCrCrUrGrC*mC*mG*mU | |
| RNACS6219 | TRAC5_ | mG*mC*mU*rGrGrUrArCrArCrGrGrCrArGrGrGrUrCrArGrUrUrUrUrA | 16618 |
| | P8R21_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrArUrCrCrArGrArArCrCr | |
| | | CrUrArGrUrGrUrGrArCrCrCrUrG*mC*mC*mG | |
| RNACS6220 | TRAC5_ | mG*mC*mU*rGrGrUrArCrArCrGrGrCrArGrGrGrUrCrArGrUrUrUrUrA | 16619 |
| | P13R23_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrArUrArUrCrCrArGrArAr | |
| | | CrCrCrUrArGrUrGrUrGrArCrCrCrUrGrCrCrGrUrG*mU*mA*mC | |
| RNACS6221 | TRAC5_ | mG*mC*mU*rGrGrUrArCrArCrGrGrCrArGrGrGrUrCrArGrUrUrUrUrA | 16620 |
| | P12R23_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrArUrArUrCrCrArGrArAr | |
| | | CrCrCrUrArGrUrGrUrGrArCrCrCrUrGrCrCrGrU*mG*mU*mA | |
| RNACS6222 | TRAC5_ | mG*mC*mU*rGrGrUrArCrArCrGrGrCrArGrGrGrUrCrArGrUrUrUrUrA | 16621 |
| | P11R23_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrArUrArUrCrCrArGrArAr | |
| | | CrCrCrUrArGrUrGrUrGrArCrCrCrUrGrCrCrG*mU*mG*mU | |
| RNACS6223 | TRAC5_ | mG*mC*mU*rGrGrUrArCrArCrGrGrCrArGrGrGrUrCrArGrUrUrUrUrA | 16622 |
| | P10R23_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrArUrArUrCrCrArGrArAr | |
| | | CrCrCrUrArGrUrGrUrGrArCrCrCrUrGrCrC*mG*mU*mG | |
| RNACS6224 | TRAC5_ | mG*mC*mU*rGrGrUrArCrArCrGrGrCrArGrGrGrUrCrArGrUrUrUrUrA | 16623 |
| | P9R23_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrArUrArUrCrCrArGrArAr | |
| | | CrCrCrUrArGrUrGrUrGrArCrCrCrUrGrC*mC*mG*mU | |
| RNACS6225 | TRAC5_ | mG*mC*mU*rGrGrUrArCrArCrGrGrCrArGrGrGrUrCrArGrUrUrUrUrA | 16624 |
| | P8R23_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrArUrArUrCrCrArGrArAr | |
| | | CrCrCrUrArGrUrGrUrGrArCrCrCrUrG*mC*mC*mG | |
| RNACS6226 | TRAC5_ | mG*mC*mU*rGrGrUrArCrArCrGrGrCrArGrGrGrUrCrArGrUrUrUrUrA | 16625 |
| | P13R25_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArCrArGrArUrArUrCrCrArGr | |
| | | ArArCrCrCrUrArGrUrGrUrGrArCrCrCrUrGrCrCrGrUrG*mU*mA*mC | |
| RNACS6227 | TRAC5_ | mG*mC*mU*rGrGrUrArCrArCrGrGrCrArGrGrGrUrCrArGrUrUrUrUrA | 16626 |
| | P12R25_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArCrArGrArUrArUrCrCrArGr | |
| | | ArArCrCrCrUrArGrUrGrUrGrArCrCrCrUrGrCrCrGrU*mG*mU*mA | |
| RNACS6228 | TRAC5_ | mG*mC*mU*rGrGrUrArCrArCrGrGrCrArGrGrGrUrCrArGrUrUrUrUrA | 16627 |
| | P11R25_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArCrArGrArUrArUrCrCrArGr | |
| | | ArArCrCrCrUrArGrUrGrUrGrArCrCrCrUrGrCrCrG*mU*mG*mU | |
| RNACS6229 | TRAC5_ | mG*mC*mU*rGrGrUrArCrArCrGrGrCrArGrGrGrUrCrArGrUrUrUrUrA | 16628 |
| | P10R25_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArCrArGrArUrArUrCrCrArGr | |
| | | ArArCrCrCrUrArGrUrGrUrGrArCrCrCrUrGrCrC*mG*mU*mG | |
| RNACS6230 | TRAC5_ | mG*mC*mU*rGrGrUrArCrArCrGrGrCrArGrGrGrUrCrArGrUrUrUrUrA | 16629 |
| | P9R25_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArCrArGrArUrArUrCrCrArGr | |
| | | ArArCrCrCrUrArGrUrGrUrGrArCrCrCrUrGrC*mC*mG*mU | |
| RNACS6231 | TRAC5_ | mG*mC*mU*rGrGrUrArCrArCrGrGrCrArGrGrGrUrCrArGrUrUrUrUrA | 16630 |
| | P8R25_ | rGrAmGmCmUmAmGmAmAmAmUmAmGmCrArArGrUrUrArArArArUrArArGr | |
| | ins5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArCrArGrArUrArUrCrCrArGr | |
| | | ArArCrCrCrUrArGrUrGrUrGrArCrCrCrUrG*mC*mC*mG | |
| RNACS6232 | TRAC5_ | mG*mC*mU*rGrGrUrArCrArCrGrGrCrArGrGrGrUrCrArGrUrUrUrUrA | 16631 |
| | P13R15_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArArCrCrCrUrArGrUrUrGrAr | |
| | | UrGrArCrCrCrUrGrCrCrGrUrG*mU*mA*mC | |
| RNACS6233 | TRAC5_ | mG*mC*mU*rGrGrUrArCrArCrGrGrCrArGrGrGrUrCrArGrUrUrUrUrA | 16632 |
| | P12R15_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArArCrCrCrUrArGrUrUrGrAr | |
| | | UrGrArCrCrCrUrGrCrCrGrU*mG*mU*mA | |
| RNACS6234 | TRAC5_ | mG*mC*mU*rGrGrUrArCrArCrGrGrCrArGrGrGrUrCrArGrUrUrUrUrA | 16633 |
| | P11R15_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArArCrCrCrUrArGrUrUrGrAr | |
| | | UrGrArCrCrCrUrGrCrCrG*mU*mG*mU | |
| RNACS6235 | TRAC5_ | mG*mC*mU*rGrGrUrArCrArCrGrGrCrArGrGrGrUrCrArGrUrUrUrUrA | 16634 |
| | P10R15_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArArCrCrCrUrArGrUrUrGrAr | |
| | | UrGrArCrCrCrUrGrCrC*mG*mU*mG | |
| RNACS6236 | TRAC5_ | mG*mC*mU*rGrGrUrArCrArCrGrGrCrArGrGrGrUrCrArGrUrUrUrUrA | 16635 |
| | P9R15_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArArCrCrCrUrArGrUrUrGrAr | |
| | | UrGrArCrCrCrUrGrC*mC*mG*mU | |
| RNACS6237 | TRAC5_ | mG*mC*mU*rGrGrUrArCrArCrGrGrCrArGrGrGrUrCrArGrUrUrUrUrA | 16636 |
| | P8R15_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArArCrCrCrUrArGrUrUrGrAr | |
| | | UrGrArCrCrCrUrG*mC*mC*mG | |
| RNACS6238 | TRAC5_ | mG*mC*mU*rGrGrUrArCrArCrGrGrCrArGrGrGrUrCrArGrUrUrUrUrA | 16637 |
| | P13R17_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrArArCrCrCrUrArGrUrUr | |
| | | GrArUrGrArCrCrCrUrGrCrCrGrUrG*mU*mA*mC | |
| RNACS6239 | TRAC5_ | mG*mC*mU*rGrGrUrArCrArCrGrGrCrArGrGrGrUrCrArGrUrUrUrUrA | 16638 |
| | P12R17_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrArArCrCrCrUrArGrUrUr | |
| | | GrArUrGrArCrCrCrUrGrCrCrGrU*mG*mU*mA | |
| RNACS6240 | TRAC5_ | mG*mC*mU*rGrGrUrArCrArCrGrGrCrArGrGrGrUrCrArGrUrUrUrUrA | 16639 |
| | P11R17_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrArArCrCrCrUrArGrUrUr | |
| | | GrArUrGrArCrCrCrUrGrCrCrG*mU*mG*mU | |
| RNACS6241 | TRAC5_ | mG*mC*mU*rGrGrUrArCrArCrGrGrCrArGrGrGrUrCrArGrUrUrUrUrA | 16640 |
| | P10R17_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrArArCrCrCrUrArGrUrUr | |
| | | GrArUrGrArCrCrCrUrGrCrC*mG*mU*mG | |
| RNACS6242 | TRAC5_ | mG*mC*mU*rGrGrUrArCrArCrGrGrCrArGrGrGrUrCrArGrUrUrUrUrA | 16641 |
| | P9R17_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrArArCrCrCrUrArGrUrUr | |
| | | GArUrGrArCrCrCrUrGrC*mC*mG*mU | |
| RNACS6243 | TRAC5_ | mG*mC*mU*rGrGrUrArCrArCrGrGrCrArGrGrGrUrCrArGrUrUrUrUrA | 16642 |
| | P8R17_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrArArCrCrCrUrArGrUrUr | |
| | | GrArUrGrArCrCrCrUrG*mC*mC*mG | |
| RNACS6244 | TRAC5_ | mG*mC*mU*rGrGrUrArCrArCrGrGrCrArGrGrGrUrCrArGrUrUrUrUrA | 16643 |
| | P13R19_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrArGrArArCrCrCrUrArGr | |
| | | UrUrGrArUrGrArCrCrCrUrGrCrCrGrUrG*mU*mA*mC | |
| RNACS6245 | TRAC5_ | mG*mC*mU*rGrGrUrArCrArCrGrGrCrArGrGrGrUrCrArGrUrUrUrUrA | 16644 |
| | P12R19_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrArGrArArCrCrCrUrArGr | |
| | | UrUrGrArUrGrArCrCrCrUrGrCrCrGrU*mG*mU*mA | |
| RNACS6246 | TRAC5_ | mG*mC*mU*rGrGrUrArCrArCrGrGrCrArGrGrGrUrCrArGrUrUrUrUrA | 16645 |
| | P11R19_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrArGrArArCrCrCrUrArGr | |
| | | UrUrGrArUrGrArCrCrCrUrGrCrCrG*mU*mG*mU | |
| RNACS6247 | TRAC5_ | mG*mC*mU*rGrGrUrArCrArCrGrGrCrArGrGrGrUrCrArGrUrUrUrUrA | 16646 |
| | P10R19_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrArGrArArCrCrCrUrArGr | |
| | | UrUrGrArUrGrArCrCrCrUrGrCrC*mG*mU*mG | |
| RNACS6248 | TRAC5_ | mG*mC*mU*rGrGrUrArCrArCrGrGrCrArGrGrGrUrCrArGrUrUrUrUrA | 16647 |
| | POR19_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrArGrArArCrCrCrUrArGr | |
| | | UrUrGrArUrGrArCrCrCrUrGrC*mC*mG*mU | |
| RNACS6249 | TRAC5_ | mG*mC*mU*rGrGrUrArCrArCrGrGrCrArGrGrGrUrCrArGrUrUrUrUrA | 16648 |
| | P8R19_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrArGrArArCrCrCrUrArGr | |
| | | UrUrGrArUrGrArCrCrCrUrG*mC*mC*mG | |
| RNACS6250 | TRAC5_ | mG*mC*mU*rGrGrUrArCrArCrGrGrCrArGrGrGrUrCrArGrUrUrUrUrA | 16649 |
| | P13R21_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrCrCrArGrArArCrCrCrUr | |
| | | ArGrUrUrGrArUrGrArCrCrCrUrGrCrCrGrUrG*mU*mA*mC | |
| RNACS6251 | TRAC5_ | mG*mC*mU*rGrGrUrArCrArCrGrGrCrArGrGrGrUrCrArGrUrUrUrUrA | 16650 |
| | P12R21_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrCrCrArGrArArCrCrCrUr | |
| | | ArGrUrUrGrArUrGrArCrCrCrUrGrCrCrGrU*mG*mU*mA | |
| RNACS6252 | TRAC5_ | mG*mC*mU*rGrGrUrArCrArCrGrGrCrArGrGrGrUrCrArGrUrUrUrUrA | 16651 |
| | P11R21_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrCrCrArGrArArCrCrCrUr | |
| | | ArGrUrUrGrArUrGrArCrCrCrUrGrCrCrG*mU*mG*mU | |
| RNACS6253 | TRAC5_ | mG*mC*mU*rGrGrUrArCrArCrGrGrCrArGrGrGrUrCrArGrUrUrUrUrA | 16652 |
| | P10R21_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrCrCrArGrArArCrCrCrUr | |
| | | ArGrUrUrGrArUrGrArCrCrCrUrGrCrC*mG*mU*mG | |
| RNACS6254 | TRAC5_ | mG*mC*mU*rGrGrUrArCrArCrGrGrCrArGrGrGrUrCrArGrUrUrUrUrA | 16653 |
| | P9R21_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrCrCrArGrArArCrCrCrUr | |
| | | ArGrUrUrGrArUrGrArCrCrCrUrGrC*mC*mG*mU | |
| RNACS6255 | TRAC5_ | mG*mC*mU*rGrGrUrArCrArCrGrGrCrArGrGrGrUrCrArGrUrUrUrUrA | 16654 |
| | P8R21_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrCrCrArGrArArCrCrCrUr | |
| | | ArGrUrUrGrArUrGrArCrCrCrUrG*mC*mC*mG | |
| RNACS6256 | TRAC5_ | mG*mC*mU*rGrGrUrArCrArCrGrGrCrArGrGrGrUrCrArGrUrUrUrUrA | 16655 |
| | P13R23_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrArUrCrCrArGrArArCrCr | |
| | | CrUrArGrUrUrGrArUrGrArCrCrCrUrGrCrCrGrUrG*mU*mA*mC | |
| RNACS6257 | TRAC5_ | mG*mC*mU*rGrGrUrArCrArCrGrGrCrArGrGrGrUrCrArGrUrUrUrUrA | 16656 |
| | P12R23_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrArUrCrCrArGrArArCrCr | |
| | | CrUrArGrUrUrGrArUrGrArCrCrCrUrGrCrCrGrU*mG*mU*mA | |
| RNACS6258 | TRAC5_ | mG*mC*mU*rGrGrUrArCrArCrGrGrCrArGrGrGrUrCrArGrUrUrUrUrA | 16657 |
| | P11R23_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrArUrCrCrArGrArArCrCr | |
| | | CrUrArGrUrUrGrArUrGrArCrCrCrUrGrCrCrG*mU*mG*mU | |
| RNACS6259 | TRAC5_ | mG*mC*mU*rGrGrUrArCrArCrGrGrCrArGrGrGrUrCrArGrUrUrUrUrA | 16658 |
| | P10R23_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrArUrCrCrArGrArArCrCr | |
| | | CrUrArGrUrUrGrArUrGrArCrCrCrUrGrCrC*mG*mU*mG | |
| RNACS6260 | TRAC5_ | mG*mC*mU*rGrGrUrArCrArCrGrGrCrArGrGrGrUrCrArGrUrUrUrUrA | 16659 |
| | P9R23_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrArUrCrCrArGrArArCrCr | |
| | | CrUrArGrUrUrGrArUrGrArCrCrCrUrGrC*mC*mG*mU | |
| RNACS6261 | TRAC5_ | mG*mC*mU*rGrGrUrArCrArCrGrGrCrArGrGrGrUrCrArGrUrUrUrUrA | 16660 |
| | P8R23_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArUrArUrCrCrArGrArArCrCr | |
| | | CrUrArGrUrUrGrArUrGrArCrCrCrUrG*mC*mC*mG | |
| RNACS6262 | TRAC5_ | mG*mC*mU*rGrGrUrArCrArCrGrGrCrArGrGrGrUrCrArGrUrUrUrUrA | 16661 |
| | P13R25_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrArUrArUrCrCrArGrArAr | |
| | | CrCrCrUrArGrUrUrGrArUrGrArCrCrCrUrGrCrCrGrUrG*mU*mA*mC | |
| RNACS6263 | TRAC5_ | mG*mC*mU*rGrGrUrArCrArCrGrGrCrArGrGrGrUrCrArGrUrUrUrUrA | 16662 |
| | P12R25_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrArUrArUrCrCrArGrArAr | |
| | | CrCrCrUrArGrUrUrGrArUrGrArCrCrCrUrGrCrCrGrU*mG*mU*mA | |
| RNACS6264 | TRAC5_ | mG*mC*mU*rGrGrUrArCrArCrGrGrCrArGrGrGrUrCrArGrUrUrUrUrA | 16663 |
| | P11R25_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrArUrArUrCrCrArGrArAr | |
| | | CrCrCrUrArGrUrUrGrArUrGrArCrCrCrUrGrCrCrG*mU*mG*mU | |
| RNACS6265 | TRAC5_ | mG*mC*mU*rGrGrUrArCrArCrGrGrCrArGrGrGrUrCrArGrUrUrUrUrA | 16664 |
| | P10R25_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrArUrArUrCrCrArGrArAr | |
| | | CrCrCrUrArGrUrUrGrArUrGrArCrCrCrUrGrCrC*mG*mU*mG | |
| RNACS6266 | TRAC5_ | mG*mC*mU*rGrGrUrArCrArCrGrGrCrArGrGrGrUrCrArGrUrUrUrUrA | 16665 |
| | P9R25_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrArUrArUrCrCrArGrArAr | |
| | | CrCrCrUrArGrUrUrGrArUrGrArCrCrCrUrGrC*mC*mG*mU | |
| RNACS6267 | TRAC5_ | mG*mC*mU*rGrGrUrArCrArCrGrGrCrArGrGrGrUrCrArGrUrUrUrUrA | 16666 |
| | P8R25_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArGrArUrArUrCrCrArGrArAr | |
| | | CrCrCrUrArGrUrUrGrArUrGrArCrCrCrUrG*mC*mC*mG | |
| RNACS6268 | TRAC5_ | mG*mC*mU*rGrGrUrArCrArCrGrGrCrArGrGrGrUrCrArGrUrUrUrUrA | 16667 |
| | P13R27_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArCrArGrArUrArUrCrCrArGr | |
| | | ArArCrCrCrUrArGrUrUrGrArUrGrArCrCrCrUrGrCrCrGrUrG*mU*mA | |
| | | *mC | |
| RNACS6269 | TRAC5_ | mG*mC*mU*rGrGrUrArCrArCrGrGrCrArGrGrGrUrCrArGrUrUrUrUrA | 16668 |
| | P12R27_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArCrArGrArUrArUrCrCrArGr | |
| | | ArArCrCrCrUrArGrUrUrGrArUrGrArCrCrCrUrGrCrCrGrU*mG*mU*m | |
| | | A | |
| RNACS6270 | TRAC5_ | mG*mC*mU*rGrGrUrArCrArCrGrGrCrArGrGrGrUrCrArGrUrUrUrUrA | 16669 |
| | P11R27_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArCrArGrArUrArUrCrCrArGr | |
| | | ArArCrCrCrUrArGrUrUrGrArUrGrArCrCrCrUrGrCrCrG*mUmG*mU | |
| RNACS6271 | TRAC5_ | mG*mC*mU*rGrGrUrArCrArCrGrGrCrArGrGrGrUrCrArGrUrUrUrUrA | 16670 |
| | P10R27_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArCrArGrArUrArUrCrCrArGr | |
| | | ArArCrCrCrUrArGrUrUrGrArUrGrArCrCrCrUrGrCrC*mG*mU*mG | |
| RNACS6272 | TRAC5_ | mG*mC*mU*rGrGrUrArCrArCrGrGrCrArGrGrGrUrCrArGrUrUrUrUrA | 16671 |
| | POR27_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArCrArGrArUrArUrCrCrArGr | |
| | | ArArCrCrCrUrArGrUrUrGrArUrGrArCrCrCrUrGrC*mC*mG*mU | |
| RNACS6273 | TRAC5_ | mG*mC*mU*rGrGrUrArCrArCrGrGrCrArGrGrGrUrCrArGrUrUrUrUrA | 16672 |
| | P8R27_ | rGrAmGmCmUmAmGmAmAmAmUmAmGmCrArArGrUrUrArArArArUrArArGr | |
| | ins7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrArCrArGrArUrArUrCrCrArGr | |
| | | ArArCrCrCrUrArGrUrUrGrArUrGrArCrCrCrUrG*mC*mC*mG | |
| RNACS6274 | TRAC5_ | mG*mC*mU*rGrGrUrArCrArCrGrGrCrArGrGrGrUrCrArGrUrUrUrUrA | 16673 |
| | P13R8_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrCrArGrUrGrArCrCrCrUr | |
| | | GrCrCrGrUrG*mU*mA*mC | |
| RNACS6275 | TRAC5_ | mG*mC*mU*rGrGrUrArCrArCrGrGrCrArGrGrGrUrCrArGrUrUrUrUrA | 16674 |
| | P12R8_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrCrArGrUrGrArCrCrCrUr | |
| | | GrCrCrGrU*mG*mU*mA | |
| RNACS6276 | TRAC5_ | mG*mC*mU*rGrGrUrArCrArCrGrGrCrArGrGrGrUrCrArGrUrUrUrUrA | 16675 |
| | P11R8_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrCrArGrUrGrArCrCrCrUr | |
| | | GrCrCrG*mU*mG*mU | |
| RNACS6277 | TRAC5_ | mG*mC*mU*rGrGrUrArCrArCrGrGrCrArGrGrGrUrCrArGrUrUrUrUrA | 16676 |
| | P10R8_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrCrArGrUrGrArCrCrCrUr | |
| | | GrCrC*mG*mU*mG | |
| RNACS6278 | TRAC5_ | mG*mC*mU*rGrGrUrArCrArCrGrGrCrArGrGrGrUrCrArGrUrUrUrUrA | 16677 |
| | P9R8_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrCrArGrUrGrArCrCrCrUr | |
| | | GrC*mC*mG*mU | |
| RNACS6279 | TRAC5_ | mG*mC*mU*rGrGrUrArCrArCrGrGrCrArGrGrGrUrCrArGrUrUrUrUrA | 16678 |
| | P8R8_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrCrCrArGrUrGrArCrCrCrUr | |
| | | G*mC*mC*mG | |
| RNACS6280 | TRAC5_ | mG*mC*mU*rGrGrUrArCrArCrGrGrCrArGrGrGrUrCrArGrUrUrUrUrA | 16679 |
| | P13R10_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrArUrCrCrArGrUrGrArCrCr | |
| | | CrUrGrCrCrGrUrG*mU*mA*mC | |
| RNACS6281 | TRAC5_ | mG*mC*mU*rGrGrUrArCrArCrGrGrCrArGrGrGrUrCrArGrUrUrUrUrA | 16680 |
| | P12R10_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrArUrCrCrArGrUrGrArCrCr | |
| | | CrUrGrCrCrGrU*mG*mU*mA | |
| RNACS6282 | TRAC5_ | mG*mC*mU*rGrGrUrArCrArCrGrGrCrArGrGrGrUrCrArGrUrUrUrUrA | 16681 |
| | P11R10_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrArUrCrCrArGrUrGrArCrCr | |
| | | CrUrGrCrCrG*mU*mG*mU | |
| RNACS6283 | TRAC5_ | mG*mC*mU*rGrGrUrArCrArCrGrGrCrArGrGrGrUrCrArGrUrUrUrUrA | 16682 |
| | P10R10_ | rGrAmGmCmUmAmGmAmAmAmUmAmGmCrArArGrUrUrArArArArUrArArGr | |
| | de15- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrArUrCrCrArGrUrGrArCrCr | |
| | | CrUrGrCrC*mG*mU*mG | |
| RNACS6284 | TRAC5_ | mG*mC*mU*rGrGrUrArCrArCrGrGrCrArGrGrGrUrCrArGrUrUrUrUrA | 16683 |
| | P9R10_ | rGrAmGmCmUmAmGmAmAmAmUmAmGmCrArArGrUrUrArArArArUrArArGr | |
| | de15- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrArUrCrCrArGrUrGrArCrCr | |
| | | CrUrGrC*mC*mG*mU | |
| RNACS6285 | TRAC5_ | mG*mC*mU*rGrGrUrArCrArCrGrGrCrArGrGrGrUrCrArGrUrUrUrUrA | 16684 |
| | P8R10_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrArUrCrCrArGrUrGrArCrCr | |
| | | CrUrG*mC*mC*mG | |
| RNACS6286 | TRAC5_ | mG*mC*mU*rGrGrUrArCrArCrGrGrCrArGrGrGrUrCrArGrUrUrUrUrA | 16685 |
| | P13R12_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrArUrArUrCrCrArGrUrGrAr | |
| | | CrCrCrUrGrCrCrGrUrG*mU*mA*mC | |
| RNACS6287 | TRAC5_ | mG*mC*mU*rGrGrUrArCrArCrGrGrCrArGrGrGrUrCrArGrUrUrUrUrA | 16686 |
| | P12R12_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrArUrArUrCrCrArGrUrGrAr | |
| | | CrCrCrUrGrCrCrGrU*mG*mU*mA | |
| RNACS6288 | TRAC5_ | mG*mC*mU*rGrGrUrArCrArCrGrGrCrArGrGrGrUrCrArGrUrUrUrUrA | 16687 |
| | P11R12_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrArUrArUrCrCrArGrUrGrAr | |
| | | CrCrCrUrGrCrCrG*mU*mG*mU | |
| RNACS6289 | TRAC5_ | mG*mC*mU*rGrGrUrArCrArCrGrGrCrArGrGrGrUrCrArGrUrUrUrUrA | 16688 |
| | P10R12_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrArUrArUrCrCrArGrUrGrAr | |
| | | CrCrCrUrGrCrC*mG*mU*mG | |
| RNACS6290 | TRAC5_ | mG*mC*mU*rGrGrUrArCrArCrGrGrCrArGrGrGrUrCrArGrUrUrUrUrA | 16689 |
| | P9R12_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrArUrArUrCrCrArGrUrGrAr | |
| | | CrCrCrUrGrC*mC*mG*mU | |
| RNACS6291 | TRAC5_ | mG*mC*mU*rGrGrUrArCrArCrGrGrCrArGrGrGrUrCrArGrUrUrUrUrA | 16690 |
| | P8R12_ | rGrAmGmCmUmAmGmAmAmAmUmAmGmCrArArGrUrUrArArArArUrArArGr | |
| | de15- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrArUrArUrCrCrArGrUrGrAr | |
| | | CrCrCrUrG*mC*mC*mG | |
| RNACS6292 | TRAC5_ | mG*mC*mU*rGrGrUrArCrArCrGrGrCrArGrGrGrUrCrArGrUrUrUrUrA | 16691 |
| | P13R14_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArGrArUrArUrCrCrArGrUr | |
| | | GrArCrCrCrUrGrCrCrGrUrG*mU*mA*mC | |
| RNACS6293 | TRAC5_ | mG*mC*mU*rGrGrUrArCrArCrGrGrCrArGrGrGrUrCrArGrUrUrUrUrA | 16692 |
| | P12R14_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArGrArUrArUrCrCrArGrUr | |
| | | GrArCrCrCrUrGrCrCrGrU*mG*mU*mA | |
| RNACS6294 | TRAC5_ | mG*mC*mU*rGrGrUrArCrArCrGrGrCrArGrGrGrUrCrArGrUrUrUrUrA | 16693 |
| | P11R14_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArGrArUrArUrCrCrArGrUr | |
| | | GrArCrCrCrUrGrCrCrG*mU*mG*mU | |
| RNACS6295 | TRAC5_ | mG*mC*mU*rGrGrUrArCrArCrGrGrCrArGrGrGrUrCrArGrUrUrUrUrA | 16694 |
| | P10R14_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArGrArUrArUrCrCrArGrUr | |
| | | GrArCrCrCrUrGrCrC*mG*mU*mG | |
| RNACS6296 | TRAC5_ | mG*mC*mU*rGrGrUrArCrArCrGrGrCrArGrGrGrUrCrArGrUrUrUrUrA | 16695 |
| | P9R14_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArGrArUrArUrCrCrArGrUr | |
| | | GrArCrCrCrUrGrC*mC*mG*mU | |
| RNACS6297 | TRAC5_ | mG*mC*mU*rGrGrUrArCrArCrGrGrCrArGrGrGrUrCrArGrUrUrUrUrA | 16696 |
| | P8R14_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArGrArUrArUrCrCrArGrUr | |
| | | GrArCrCrCrUrG*mC*mC*mG | |
| RNACS6298 | TRAC5_ | mG*mC*mU*rGrGrUrArCrArCrGrGrCrArGrGrGrUrCrArGrUrUrUrUrA | 16697 |
| | P13R16_del5- | rGrAmGmCmUmAmGmAmAmAmUmAmGmCrArArGrUrUrArArArArUrArArGr | |
| | PAM | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrArCrArGrArUrArUrCrCrAr | |
| | | GrUrGrArCrCrCrUrGrCrCrGrUrG*mU*mA*mC | |
| RNACS6299 | TRAC5_ | mG*mC*mU*rGrGrUrArCrArCrGrGrCrArGrGrGrUrCrArGrUrUrUrUrA | 16698 |
| | P12R16_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArCrArGrArUrArUrCrCrAr | |
| | | GrUrGrArCrCrCrUrGrCrCrGrU*mG*mU*mA | |
| RNACS6300 | TRAC5_ | mG*mC*mU*rGrGrUrArCrArCrGrGrCrArGrGrGrUrCrArGrUrUrUrUrA | 16699 |
| | P11R16_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArCrArGrArUrArUrCrCrAr | |
| | | GrUrGrArCrCrCrUrGrCrCrG*mU*mG*mU | |
| RNACS6301 | TRAC5_ | mG*mC*mU*rGrGrUrArCrArCrGrGrCrArGrGrGrUrCrArGrUrUrUrUrA | 16700 |
| | P10R16_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArCrArGrArUrArUrCrCrAr | |
| | | GrUrGrArCrCrCrUrGrCrC*mG*mU*mG | |
| RNACS6302 | TRAC5_ | mG*mC*mU*rGrGrUrArCrArCrGrGrCrArGrGrGrUrCrArGrUrUrUrUrA | 16701 |
| | P9R16_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArCrArGrArUrArUrCrCrAr | |
| | | GrUrGrArCrCrCrUrGrC*mC*mG*mU | |
| RNACS6303 | TRAC5_ | mG*mC*mU*rGrGrUrArCrArCrGrGrCrArGrGrGrUrCrArGrUrUrUrUrA | 16702 |
| | P8R16_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArCrArGrArUrArUrCrCrAr | |
| | | GrUrGrArCrCrCrUrG*mC*mC*mG | |
| RNACS6304 | TRAC5_ | mG*mC*mU*rGrGrUrArCrArCrGrGrCrArGrGrGrUrCrArGrUrUrUrUrA | 16703 |
| | P13R18_del5- | rGrAmGmCmUmAmGmAmAmAmUmAmGmCrArArGrUrUrArArArArUrArArGr | |
| | PAM | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrCrCrArCrArGrArUrArUrCr | |
| | | CrArGrUrGrArCrCrCrUrGrCrCrGrUrG*mU*mA*mC | |
| RNACS6305 | TRAC5_ | mG*mC*mU*rGrGrUrArCrArCrGrGrCrArGrGrGrUrCrArGrUrUrUrUrA | 16704 |
| | P12R18_del5- | rGrAmGmCmUmAmGmAmAmAmUmAmGmCrArArGrUrUrArArArArUrArArGr | |
| | PAM | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrCrCrArCrArGrArUrArUrCr | |
| | | CrArGrUrGrArCrCrCrUrGrCrCrGrU*mG*mU*mA | |
| RNACS6306 | TRAC5_ | mG*mC*mU*rGrGrUrArCrArCrGrGrCrArGrGrGrUrCrArGrUrUrUrUrA | 16705 |
| | P11R18_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrCrArCrArGrArUrArUrCr | |
| | | CrArGrUrGrArCrCrCrUrGrCrCrG*mU*mG*mU | |
| RNACS6307 | TRAC5_ | mG*mC*mU*rGrGrUrArCrArCrGrGrCrArGrGrGrUrCrArGrUrUrUrUrA | 16706 |
| | P10R18_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrCrArCrArGrArUrArUrCr | |
| | | CrArGrUrGrArCrCrCrUrGrCrC*mG*mU*mG | |
| RNACS6308 | TRAC5_ | mG*mC*mU*rGrGrUrArCrArCrGrGrCrArGrGrGrUrCrArGrUrUrUrUrA | 16707 |
| | P9R18_del5- | rGrAmGmCmUmAmGmAmAmAmUmAmGmCrArArGrUrUrArArArArUrArArGr | |
| | PAM | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrCrCrArCrArGrArUrArUrCr | |
| | | CrArGrUrGrArCrCrCrUrGrC*mC*mG*mU | |
| RNACS6309 | TRAC5_ | mG*mC*mU*rGrGrUrArCrArCrGrGrCrArGrGrGrUrCrArGrUrUrUrUrA | 16708 |
| | P8R18_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrCrArCrArGrArUrArUrCr | |
| | | CrArGrUrGrArCrCrCrUrG*mC*mC*mG | |
| RNACS6310 | TRAC5_ | mG*mC*mU*rGrGrUrArCrArCrGrGrCrArGrGrGrUrCrArGrUrUrUrUrA | 16709 |
| | P13R20_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrUrCrCrCrArCrArGrArUrAr | |
| | | UrCrCrArGrUrGrArCrCrCrUrGrCrCrGrUrG*mU*mA*mC | |
| RNACS6311 | TRAC5_ | mG*mC*mU*rGrGrUrArCrArCrGrGrCrArGrGrGrUrCrArGrUrUrUrUrA | 16710 |
| | P12R20_ | rGrAmGmCmUmAmGmAmAmAmUmAmGmCrArArGrUrUrArArArArUrArArGr | |
| | de15- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrUrCrCrCrArCrArGrArUrAr | |
| | | UrCrCrArGrUrGrArCrCrCrUrGrCrCrGrUmG*mU*mA | |
| RNACS6312 | TRAC5_ | mG*mC*mU*rGrGrUrArCrArCrGrGrCrArGrGrGrUrCrArGrUrUrUrUrA | 16711 |
| | P11R20_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrUrCrCrCrArCrArGrArUrAr | |
| | | UrCrCrArGrUrGrArCrCrCrUrGrCrCrG*mU*mG*mU | |
| RNACS6313 | TRAC5_ | mG*mC*mU*rGrGrUrArCrArCrGrGrCrArGrGrGrUrCrArGrUrUrUrUrA | 16712 |
| | P10R20_ | rGrAmGmCmUmAmGmAmAmAmUmAmGmCrArArGrUrUrArArArArUrArArGr | |
| | del5- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrUrCrCrCrArCrArGrArUrAr | |
| | | UrCrCrArGrUrGrArCrCrCrUrGrCrC*mG*mU*mG | |
| RNACS6314 | TRAC5_ | mG*mC*mU*rGrGrUrArCrArCrGrGrCrArGrGrGrUrCrArGrUrUrUrUrA | 16713 |
| | P9R20_ | rGrAmGmCmUmAmGmAmAmAmUmAmGmCrArArGrUrUrArArArArUrArArGr | |
| | de15- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrUrCrCrCrArCrArGrArUrAr | |
| | | UrCrCrArGrUrGrArCrCrCrUrGrC*mC*mG*mU | |
| RNACS6315 | TRAC5_ | mG*mC*mU*rGrGrUrArCrArCrGrGrCrArGrGrGrUrCrArGrUrUrUrUrA | 16714 |
| | P8R20_ | rGrAmGmCmUmAmGmAmAmAmUmAmGmCrArArGrUrUrArArArArUrArArGr | |
| | de15- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrUrCrCrCrArCrArGrArUrAr | |
| | | UrCrCrArGrUrGrArCrCrCrUrG*mC*mC*mG | |
| RNACS6316 | TRAC5_ | mG*mC*mU*rGrGrUrArCrArCrGrGrCrArGrGrGrUrCrArGrUrUrUrUrA | 16715 |
| | P13R8_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrArUrCrCrUrGrArCrCrCrUr | |
| | | GrCrCrGrUrG*mU*mA*mC | |
| RNACS6317 | TRAC5_ | mG*mC*mU*rGrGrUrArCrArCrGrGrCrArGrGrGrUrCrArGrUrUrUrUrA | 16716 |
| | P12R8_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrArUrCrCrUrGrArCrCrCrUr | |
| | | GrCrCrGrU*mG*mU*mA | |
| RNACS6318 | TRAC5_ | mG*mC*mU*rGrGrUrArCrArCrGrGrCrArGrGrGrUrCrArGrUrUrUrUrA | 16717 |
| | P11R8_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrArUrCrCrUrGrArCrCrCrUr | |
| | | GrCrCrG*mU*mG*mU | |
| RNACS6319 | TRAC5_ | mG*mC*mU*rGrGrUrArCrArCrGrGrCrArGrGrGrUrCrArGrUrUrUrUrA | 16718 |
| | P10R8_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrArUrCrCrUrGrArCrCrCrUr | |
| | | GrCrC*mG*mU*mG | |
| RNACS6320 | TRAC5_ | mG*mC*mU*rGrGrUrArCrArCrGrGrCrArGrGrGrUrCrArGrUrUrUrUrA | 16719 |
| | P9R8_del7- | rGrAmGmCmUmAmGmAmAmAmUmAmGmCrArArGrUrUrArArArArUrArArGr | |
| | PAM | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrUrArUrCrCrUrGrArCrCrCrUr | |
| | | GrC*mC*mG*mU | |
| RNACS6321 | TRAC5_ | mG*mC*mU*rGrGrUrArCrArCrGrGrCrArGrGrGrUrCrArGrUrUrUrUrA | 16720 |
| | P8R8_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrArUrCrCrUrGrArCrCrCrUr | |
| | | G*mC*mC*mG | |
| RNACS6322 | TRAC5_ | mG*mC*mU*rGrGrUrArCrArCrGrGrCrArGrGrGrUrCrArGrUrUrUrUrA | 16721 |
| | P13R10_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrArUrArUrCrCrUrGrArCrCr | |
| | | CrUrGrCrCrGrUrG*mU*mA*mC | |
| RNACS6323 | TRAC5_ | mG*mC*mU*rGrGrUrArCrArCrGrGrCrArGrGrGrUrCrArGrUrUrUrUrA | 16722 |
| | P12R10_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrArUrArUrCrCrUrGrArCrCr | |
| | | CrUrGrCrCrGrU*mG*mU*mA | |
| RNACS6324 | TRAC5_ | mG*mC*mU*rGrGrUrArCrArCrGrGrCrArGrGrGrUrCrArGrUrUrUrUrA | 16723 |
| | P11R10_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrArUrArUrCrCrUrGrArCrCr | |
| | | CrUrGrCrCrG*mU*mG*mU | |
| RNACS6325 | TRAC5_ | mG*mC*mU*rGrGrUrArCrArCrGrGrCrArGrGrGrUrCrArGrUrUrUrUrA | 16724 |
| | P10R10_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrArUrArUrCrCrUrGrArCrCr | |
| | | CrUrGrCrC*mG*mU*mG | |
| RNACS6326 | TRAC5_ | mG*mC*mU*rGrGrUrArCrArCrGrGrCrArGrGrGrUrCrArGrUrUrUrUrA | 16725 |
| | P9R10_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrArUrArUrCrCrUrGrArCrCr | |
| | | CrUrGrC*mC*mG*mU | |
| RNACS6327 | TRAC5_ | mG*mC*mU*rGrGrUrArCrArCrGrGrCrArGrGrGrUrCrArGrUrUrUrUrA | 16726 |
| | P8R10_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrArUrArUrCrCrUrGrArCrCr | |
| | | CrUrG*mC*mC*mG | |
| RNACS6328 | TRAC5_ | mG*mC*mU*rGrGrUrArCrArCrGrGrCrArGrGrGrUrCrArGrUrUrUrUrA | 16727 |
| | P13R12_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArGrArUrArUrCrCrUrGrAr | |
| | | CrCrCrUrGrCrCrGrUrG*mU*mA*mC | |
| RNACS6329 | TRAC5_ | mG*mC*mU*rGrGrUrArCrArCrGrGrCrArGrGrGrUrCrArGrUrUrUrUrA | 16728 |
| | P12R12_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArGrArUrArUrCrCrUrGrAr | |
| | | CrCrCrUrGrCrCrGrU*mG*mU*mA | |
| RNACS6330 | TRAC5_ | mG*mC*mU*rGrGrUrArCrArCrGrGrCrArGrGrGrUrCrArGrUrUrUrUrA | 16729 |
| | P11R12_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArGrArUrArUrCrCrUrGrAr | |
| | | CrCrCrUrGrCrCrG*mU*mG*mU | |
| RNACS6331 | TRAC5_ | mG*mC*mU*rGrGrUrArCrArCrGrGrCrArGrGrGrUrCrArGrUrUrUrUrA | 16730 |
| | P10R12_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArGrArUrArUrCrCrUrGrAr | |
| | | CrCrCrUrGrCrC*mG*mU*mG | |
| RNACS6332 | TRAC5_ | mG*mC*mU*rGrGrUrArCrArCrGrGrCrArGrGrGrUrCrArGrUrUrUrUrA | 16731 |
| | P9R12_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArGrArUrArUrCrCrUrGrAr | |
| | | CrCrCrUrGrC*mC*mG*mU | |
| RNACS6333 | TRAC5_ | mG*mC*mU*rGrGrUrArCrArCrGrGrCrArGrGrGrUrCrArGrUrUrUrUrA | 16732 |
| | P8R12_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArGrArUrArUrCrCrUrGrAr | |
| | | CrCrCrUrG*mC*mC*mG | |
| RNACS6334 | TRAC5_ | mG*mC*mU*rGrGrUrArCrArCrGrGrCrArGrGrGrUrCrArGrUrUrUrUrA | 16733 |
| | P13R14_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArCrArGrArUrArUrCrCrUr | |
| | | GrArCrCrCrUrGrCrCrGrUrG*mU*mA*mC | |
| RNACS6335 | TRAC5_ | mG*mC*mU*rGrGrUrArCrArCrGrGrCrArGrGrGrUrCrArGrUrUrUrUrA | 16734 |
| | P12R14_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArCrArGrArUrArUrCrCrUr | |
| | | GrArCrCrCrUrGrCrCrGrU*mG*mU*mA | |
| RNACS6336 | TRAC5_ | mG*mC*mU*rGrGrUrArCrArCrGrGrCrArGrGrGrUrCrArGrUrUrUrUrA | 16735 |
| | P11R14_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArCrArGrArUrArUrCrCrUr | |
| | | GrArCrCrCrUrGrCrCrG*mU*mG*mU | |
| RNACS6337 | TRAC5_ | mG*mC*mU*rGrGrUrArCrArCrGrGrCrArGrGrGrUrCrArGrUrUrUrUrA | 16736 |
| | P10R14_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArCrArGrArUrArUrCrCrUr | |
| | | GrArCrCrCrUrGrCrC*mG*mU*mG | |
| RNACS6338 | TRAC5_ | mG*mC*mU*rGrGrUrArCrArCrGrGrCrArGrGrGrUrCrArGrUrUrUrUrA | 16737 |
| | POR14_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrArCrArGrArUrArUrCrCrUr | |
| | | GrArCrCrCrUrGrC*mC*mG*mU | |
| RNACS6339 | TRAC5_ | mG*mC*mU*rGrGrUrArCrArCrGrGrCrArGrGrGrUrCrArGrUrUrUrUrA | 16738 |
| | P8R14_del7- | rGrAmGmCmUmAmGmAmAmAmUmAmGmCrArArGrUrUrArArArArUrArArGr | |
| | PAM | GrCrUrArGrUrCrCrGrUrUrArUrCrAmAmCmUmUmGmAmAmAmAmAmGmUmG | |
| | | mGmCmAmCmCmGmAmGmUmCmGmGmUmGmCrCrArCrArGrArUrArUrCrCrUr | |
| | | GrArCrCrCrUrG*mC*mC*mG | |
| RNACS6340 | TRAC5_ | mG*mC*mU*rGrGrUrArCrArCrGrGrCrArGrGrGrUrCrArGrUrUrUrUrA | 16739 |
| | P13R16_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrCrArCrArGrArUrArUrCr | |
| | | CrUrGrArCrCrCrUrGrCrCrGrUrG*mU*mA*mC | |
| RNACS6341 | TRAC5_ | mG*mC*mU*rGrGrUrArCrArCrGrGrCrArGrGrGrUrCrArGrUrUrUrUrA | 16740 |
| | P12R16_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrCrArCrArGrArUrArUrCr | |
| | | CrUrGrArCrCrCrUrGrCrCrGrU*mG*mU*mA | |
| RNACS6342 | TRAC5_ | mG*mC*mU*rGrGrUrArCrArCrGrGrCrArGrGrGrUrCrArGrUrUrUrUrA | 16741 |
| | P11R16_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrCrArCrArGrArUrArUrCr | |
| | | CrUrGrArCrCrCrUrGrCrCrG*mU*mG*mU | |
| RNACS6343 | TRAC5_ | mG*mC*mU*rGrGrUrArCrArCrGrGrCrArGrGrGrUrCrArGrUrUrUrUrA | 16742 |
| | P10R16_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrCrArCrArGrArUrArUrCr | |
| | | CrUrGrArCrCrCrUrGrCrC*mG*mU*mG | |
| RNACS6344 | TRAC5_ | mG*mC*mU*rGrGrUrArCrArCrGrGrCrArGrGrGrUrCrArGrUrUrUrUrA | 16743 |
| | P9R16_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrCrArCrArGrArUrArUrCr | |
| | | CrUrGrArCrCrCrUrGrC*mC*mG*mU | |
| RNACS6345 | TRAC5_ | mG*mC*mU*rGrGrUrArCrArCrGrGrCrArGrGrGrUrCrArGrUrUrUrUrA | 16744 |
| | P8R16_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrCrCrCrArCrArGrArUrArUrCr | |
| | | CrUrGrArCrCrCrUrG*mC*mC*mG | |
| RNACS6346 | TRAC5_ | mG*mC*mU*rGrGrUrArCrArCrGrGrCrArGrGrGrUrCrArGrUrUrUrUrA | 16745 |
| | P13R18_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrUrCrCrCrArCrArGrArUrAr | |
| | | UrCrCrUrGrArCrCrCrUrGrCrCrGrUrG*mU*mA*mC | |
| RNACS6347 | TRAC5_ | mG*mC*mU*rGrGrUrArCrArCrGrGrCrArGrGrGrUrCrArGrUrUrUrUrA | 16746 |
| | P12R18_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrUrCrCrCrArCrArGrArUrAr | |
| | | UrCrCrUrGrArCrCrCrUrGrCrCrGrU*mG*mU*mA | |
| RNACS6348 | TRAC5_ | mG*mC*mU*rGrGrUrArCrArCrGrGrCrArGrGrGrUrCrArGrUrUrUrUrA | 16747 |
| | P11R18_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrUrCrCrCrArCrArGrArUrAr | |
| | | UrCrCrUrGrArCrCrCrUrGrCrCrG*mU*mG*mU | |
| RNACS6349 | TRAC5_ | mG*mC*mU*rGrGrUrArCrArCrGrGrCrArGrGrGrUrCrArGrUrUrUrUrA | 16748 |
| | P10R18_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrUrCrCrCrArCrArGrArUrAr | |
| | | UrCrCrUrGrArCrCrCrUrGrCrC*mG*mU*mG | |
| RNACS6350 | TRAC5_ | mG*mC*mU*rGrGrUrArCrArCrGrGrCrArGrGrGrUrCrArGrUrUrUrUrA | 16749 |
| | P9R18_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrGrUrCrCrCrArCrArGrArUrAr | |
| | | UrCrCrUrGrArCrCrCrUrGrC*mC*mG*mU | |
| RNACS6351 | 18_ | UmAmGmCrArArGrUrUrArArArArUrArArGrGrCrUrArGrUrCrCrGrUrU | 16750 |
| | del7- | rArUrCrAmAmCmUmUmGmAmAmAmAmAmGmUmGmGmCmAmCmCmGmAmGmUmCm | |
| | PAM | GmGmUmGmCrGrUrCrCrCrArCrArGrArUrArUrCrCrUrGrArCrCrCrUrG | |
| | | *mC*mC*mG | |
| RNACS6352 | TRAC5_ | mG*mC*mU*rGrGrUrArCrArCrGrGrCrArGrGrGrUrCrArGrUrUrUrUrA | 16751 |
| | P13R20_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrUrGrUrCrCrCrArCrArGrAr | |
| | | UrArUrCrCrUrGrArCrCrCrUrGrCrCrGrUrG*mU*mA*mC | |
| RNACS6353 | TRAC5_ | mG*mC*mU*rGrGrUrArCrArCrGrGrCrArGrGrGrUrCrArGrUrUrUrUrA | 16752 |
| | P12R20_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrUrGrUrCrCrCrArCrArGrAr | |
| | | UrArUrCrCrUrGrArCrCrCrUrGrCrCrGrU*mG*mU*mA | |
| RNACS6354 | TRAC5_ | mG*mC*mU*rGrGrUrArCrArCrGrGrCrArGrGrGrUrCrArGrUrUrUrUrA | 16753 |
| | P11R20_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrUrGrUrCrCrCrArCrArGrAr | |
| | | UrArUrCrCrUrGrArCrCrCrUrGrCrCrG*mU*mG*mU | |
| RNACS6355 | TRAC5_ | mG*mC*mU*rGrGrUrArCrArCrGrGrCrArGrGrGrUrCrArGrUrUrUrUrA | 16754 |
| | P10R20_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrUrGrUrCrCrCrArCrArGrAr | |
| | | UrArUrCrCrUrGrArCrCrCrUrGrCrC*mG*mU*mG | |
| RNACS6356 | TRAC5_ | mG*mC*mU*rGrGrUrArCrArCrGrGrCrArGrGrGrUrCrArGrUrUrUrUrA | 16755 |
| | POR20_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrUrGrUrCrCrCrArCrArGrAr | |
| | | UrArUrCrCrUrGrArCrCrCrUrGrC*mC*mG*mU | |
| RNACS6357 | TRAC5_ | mG*mC*mU*rGrGrUrArCrArCrGrGrCrArGrGrGrUrCrArGrUrUrUrUrA | 16756 |
| | P8R20_ | rGrAmGmCmUmAmGmAmAmAmUmAmGmCrArArGrUrUrArArArArUrArArGr | |
| | del7- | GrCrUrArGrUrCrCrGrUrUrArUrCrAmAmCmUmUmGmAmAmAmAmAmGmUmG | |
| | PAM | mGmCmAmCmCmGmAmGmUmCmGmGmUmGmCrUrUrGrUrCrCrCrArCrArGrAr | |
| | | UrArUrCrCrUrGrArCrCrCrUrG*mC*mC*mG | |

Table 2A shows the sequences of Table 1A without chemical modifications. In some embodiments, the sequences of Table 2A may be used without chemical modifications, or with one or more chemical modifications.

TABLE 2A
Table 1A Sequences without Chemical Modifications
Table 1A provides exemplary template RNAs for use in combination within a heterologous gene modifying
system described herein for disrupting, the TRAC gene using a TRAC4_gRNA spacer (gagaaucaaaaucggugaau
SEQ ID NO. 17513) or a TRAC5_gRNA spacer (gcugguacacggcaggguca SEQ ID NO. 17514). The description of
each exemplary template RNA (column 2) identifies the gRNA spacer, the length of the PBS, the length
of the heterologous object sequence, the nature and size of the edit, and the location in the
target nucleic acid sequence. For example TRAC4_3R10_ins5-nick indicates that the template RNA
comprised a TRAC4_gRNA spacer, a PBS of length 13 nucleotides, an RT region (heterologous object
sequence) of length 10 nucleotides, and was designed to produce an_insertion of 5 nucleotides near
the nick.

| | | | SEQ |
|---|---|---|---|
| | | | ID |
| Identifier | Description | Sequence | NO |
| RNACS5602 | TRAC4_ | GAGAAUCAAAAUCGGUGAAUGUUUUAGAGCUAGAAAUAGCAAGUUAAAAUAAGG | 16757 |
| | P13R10_ins5- | CUAGUCCGUUAUCAACUUGAAAAAGUGGCACCGAGUCGGUGCCUAUUUAGUGCAC | |
| | nick | CGAUUUUGAU | |
| RNACS5603 | TRAC4_ | GAGAAUCAAAAUCGGUGAAUGUUUUAGAGCUAGAAAUAGCAAGUUAAAAUAAGG | 16758 |
| | P12R10_ins5- | CUAGUCCGUUAUCAACUUGAAAAAGUGGCACCGAGUCGGUGCCUAUUUAGUGCAC | |
| | nick | CGAUUUUGA | |
| RNACS5604 | TRAC4_ | GAGAAUCAAAAUCGGUGAAUGUUUUAGAGCUAGAAAUAGCAAGUUAAAAUAAGG | 16759 |
| | P11R10_ins5- | CUAGUCCGUUAUCAACUUGAAAAAGUGGCACCGAGUCGGUGCCUAUUUAGUGCAC | |
| | nick | CGAUUUUG | |
| RNACS5605 | TRAC4_ | GAGAAUCAAAAUCGGUGAAUGUUUUAGAGCUAGAAAUAGCAAGUUAAAAUAAGG | 16760 |
| | P10R10_ins5- | CUAGUCCGUUAUCAACUUGAAAAAGUGGCACCGAGUCGGUGCCUAUUUAGUGCAC | |
| | nick | CGAUUUU | |
| RNACS5606 | TRAC4_ | GAGAAUCAAAAUCGGUGAAUGUUUUAGAGCUAGAAAUAGCAAGUUAAAAUAAGG | 16761 |
| | P9R10_ins5- | CUAGUCCGUUAUCAACUUGAAAAAGUGGCACCGAGUCGGUGCCUAUUUAGUGCAC | |
| | nick | CGAUUU | |
| RNACS5607 | TRAC4_ | GAGAAUCAAAAUCGGUGAAUGUUUUAGAGCUAGAAAUAGCAAGUUAAAAUAAGG | 16762 |
| | P8R10_ins5- | CUAGUCCGUUAUCAACUUGAAAAAGUGGCACCGAGUCGGUGCCUAUUUAGUGCAC | |
| | nick | CGAUU | |
| RNACS5608 | TRAC4_ | GAGAAUCAAAAUCGGUGAAUGUUUUAGAGCUAGAAAUAGCAAGUUAAAAUAAGG | 16763 |
| | P13R12_ins5- | CUAGUCCGUUAUCAACUUGAAAAAGUGGCACCGAGUCGGUGCGCCUAUUUAGUGC | |
| | nick | ACCGAUUUUGAU | |
| RNACS5609 | TRAC4_ | GAGAAUCAAAAUCGGUGAAUGUUUUAGAGCUAGAAAUAGCAAGUUAAAAUAAGG | 16764 |
| | P12R12_ins5- | CUAGUCCGUUAUCAACUUGAAAAAGUGGCACCGAGUCGGUGCGCCUAUUUAGUGC | |
| | nick | ACCGAUUUUGA | |
| RNACS5610 | TRAC4_ | GAGAAUCAAAAUCGGUGAAUGUUUUAGAGCUAGAAAUAGCAAGUUAAAAUAAGG | 16765 |
| | P11R12_ins5- | CUAGUCCGUUAUCAACUUGAAAAAGUGGCACCGAGUCGGUGCGCCUAUUUAGUGC | |
| | nick | ACCGAUUUUG | |
| RNACS5611 | TRAC4_ | GAGAAUCAAAAUCGGUGAAUGUUUUAGAGCUAGAAAUAGCAAGUUAAAAUAAGG | 16766 |
| | P10R12_ins5- | CUAGUCCGUUAUCAACUUGAAAAAGUGGCACCGAGUCGGUGCGCCUAUUUAGUGC | |
| | nick | ACCGAUUUU | |
| RNACS5612 | TRAC4_ | GAGAAUCAAAAUCGGUGAAUGUUUUAGAGCUAGAAAUAGCAAGUUAAAAUAAGG | 16767 |
| | P9R12_ins5- | CUAGUCCGUUAUCAACUUGAAAAAGUGGCACCGAGUCGGUGCGCCUAUUUAGUGC | |
| | nick | ACCGAUUU | |
| RNACS5613 | TRAC4_ | GAGAAUCAAAAUCGGUGAAUGUUUUAGAGCUAGAAAUAGCAAGUUAAAAUAAGG | 16768 |
| | P8R12_ins5- | CUAGUCCGUUAUCAACUUGAAAAAGUGGCACCGAGUCGGUGCGCCUAUUUAGUGC | |
| | nick | ACCGAUU | |
| RNACS5614 | TRAC4_ | GAGAAUCAAAAUCGGUGAAUGUUUUAGAGCUAGAAAUAGCAAGUUAAAAUAAGG | 16769 |
| | P13R14_ins5- | CUAGUCCGUUAUCAACUUGAAAAAGUGGCACCGAGUCGGUGCCUGCCUAUUUAGU | |
| | nick | GCACCGAUUUUGAU | |
| RNACS5615 | TRAC4_ | GAGAAUCAAAAUCGGUGAAUGUUUUAGAGCUAGAAAUAGCAAGUUAAAAUAAGG | 16770 |
| | P12R14_ins5- | CUAGUCCGUUAUCAACUUGAAAAAGUGGCACCGAGUCGGUGCCUGCCUAUUUAGU | |
| | nick | GCACCGAUUUUGA | |
| RNACS5616 | TRAC4_ | GAGAAUCAAAAUCGGUGAAUGUUUUAGAGCUAGAAAUAGCAAGUUAAAAUAAGG | 16771 |
| | P11R14_ins5- | CUAGUCCGUUAUCAACUUGAAAAAGUGGCACCGAGUCGGUGCCUGCCUAUUUAGU | |
| | nick | GCACCGAUUUUG | |
| RNACS5617 | TRAC4_ | GAGAAUCAAAAUCGGUGAAUGUUUUAGAGCUAGAAAUAGCAAGUUAAAAUAAGG | 16772 |
| | P10R14_ins5- | CUAGUCCGUUAUCAACUUGAAAAAGUGGCACCGAGUCGGUGCCUGCCUAUUUAGU | |
| | nick | GCACCGAUUUU | |
| RNACS5618 | TRAC4_ | CUAGUCCGUUAUCAACUUGAAAAAGUGGCACCGAGUCGGUGCCUGCCUAUUUAGU | 16773 |
| | P9R14_ins5- | GAGAAUCAAAAUCGGUGAAUGUUUUAGAGCUAGAAAUAGCAAGUUAAAAUAAGG | |
| | nick | GCACCGAUUU | |
| RNACS5619 | TRAC4_ | GAGAAUCAAAAUCGGUGAAUGUUUUAGAGCUAGAAAUAGCAAGUUAAAAUAAGG | 16774 |
| | P8R14_ins5- | CUAGUCCGUUAUCAACUUGAAAAAGUGGCACCGAGUCGGUGCCUGCCUAUUUAGU | |
| | nick | GCACCGAUU | |
| RNACS5620 | TRAC4_ | GAGAAUCAAAAUCGGUGAAUGUUUUAGAGCUAGAAAUAGCAAGUUAAAAUAAGG | 16775 |
| | P13R16_ins5- | CUAGUCCGUUAUCAACUUGAAAAAGUGGCACCGAGUCGGUGCGUCUGCCUAUUUA | |
| | nick | GUGCACCGAUUUUGAU | |
| RNACS5621 | TRAC4_ | GAGAAUCAAAAUCGGUGAAUGUUUUAGAGCUAGAAAUAGCAAGUUAAAAUAAGG | 16776 |
| | P12R16_ins5- | CUAGUCCGUUAUCAACUUGAAAAAGUGGCACCGAGUCGGUGCGUCUGCCUAUUUA | |
| | nick | GUGCACCGAUUUUGA | |
| RNACS5622 | TRAC4_ | GAGAAUCAAAAUCGGUGAAUGUUUUAGAGCUAGAAAUAGCAAGUUAAAAUAAGG | 16777 |
| | P11R16_ins5- | CUAGUCCGUUAUCAACUUGAAAAAGUGGCACCGAGUCGGUGCGUCUGCCUAUUUA | |
| | nick | GUGCACCGAUUUUG | |
| RNACS5623 | TRAC4_ | GAGAAUCAAAAUCGGUGAAUGUUUUAGAGCUAGAAAUAGCAAGUUAAAAUAAGG | 16778 |
| | P10R16_ins5- | CUAGUCCGUUAUCAACUUGAAAAAGUGGCACCGAGUCGGUGCGUCUGCCUAUUUA | |
| | nick | GUGCACCGAUUUU | |
| RNACS5624 | TRAC4_ | GAGAAUCAAAAUCGGUGAAUGUUUUAGAGCUAGAAAUAGCAAGUUAAAAUAAGG | 16779 |
| | P9R16_ins5- | CUAGUCCGUUAUCAACUUGAAAAAGUGGCACCGAGUCGGUGCGUCUGCCUAUUUA | |
| | nick | GUGCACCGAUUU | |
| RNACS5625 | TRAC4_ | GAGAAUCAAAAUCGGUGAAUGUUUUAGAGCUAGAAAUAGCAAGUUAAAAUAAGG | 16780 |
| | P8R16_ins5- | CUAGUCCGUUAUCAACUUGAAAAAGUGGCACCGAGUCGGUGCGUCUGCCUAUUUA | |
| | nick | GUGCACCGAUU | |
| RNACS5626 | TRAC4_ | GAGAAUCAAAAUCGGUGAAUGUUUUAGAGCUAGAAAUAGCAAGUUAAAAUAAGG | 16781 |
| | P13R18_ins5- | CUAGUCCGUUAUCAACUUGAAAAAGUGGCACCGAGUCGGUGCCUGUCUGCCUAUU | |
| | nick | UAGUGCACCGAUUUUGAU | |
| RNACS5627 | TRAC4_ | GAGAAUCAAAAUCGGUGAAUGUUUUAGAGCUAGAAAUAGCAAGUUAAAAUAAGG | 16782 |
| | P12R18_ins5- | CUAGUCCGUUAUCAACUUGAAAAAGUGGCACCGAGUCGGUGCCUGUCUGCCUAUU | |
| | nick | UAGUGCACCGAUUUUGA | |
| RNACS5628 | TRAC4_ | GAGAAUCAAAAUCGGUGAAUGUUUUAGAGCUAGAAAUAGCAAGUUAAAAUAAGG | 16783 |
| | P11R18_ins5- | CUAGUCCGUUAUCAACUUGAAAAAGUGGCACCGAGUCGGUGCCUGUCUGCCUAUU | |
| | nick | UAGUGCACCGAUUUUG | |
| RNACS5629 | TRAC4_ | GAGAAUCAAAAUCGGUGAAUGUUUUAGAGCUAGAAAUAGCAAGUUAAAAUAAGG | 16784 |
| | P10R18_ins5- | CUAGUCCGUUAUCAACUUGAAAAAGUGGCACCGAGUCGGUGCCUGUCUGCCUAUU | |
| | nick | UAGUGCACCGAUUUU | |
| RNACS5630 | TRAC4_ | GAGAAUCAAAAUCGGUGAAUGUUUUAGAGCUAGAAAUAGCAAGUUAAAAUAAGG | 16785 |
| | P9R18_ins5- | CUAGUCCGUUAUCAACUUGAAAAAGUGGCACCGAGUCGGUGCCUGUCUGCCUAUU | |
| | nick | UAGUGCACCGAUUU | |
| RNACS5631 | TRAC4_ | GAGAAUCAAAAUCGGUGAAUGUUUUAGAGCUAGAAAUAGCAAGUUAAAAUAAGG | 16786 |
| | P8R18_ins5- | CUAGUCCGUUAUCAACUUGAAAAAGUGGCACCGAGUCGGUGCCUGUCUGCCUAUU | |
| | nick | UAGUGCACCGAUU | |
| RNACS5632 | TRAC4_ | GAGAAUCAAAAUCGGUGAAUGUUUUAGAGCUAGAAAUAGCAAGUUAAAAUAAGG | 16787 |
| | P13R20_ins5- | CUAGUCCGUUAUCAACUUGAAAAAGUGGCACCGAGUCGGUGCGUCUGUCUGCCUA | |
| | nick | UUUAGUGCACCGAUUUUGAU | |
| RNACS5633 | TRAC4_ | GAGAAUCAAAAUCGGUGAAUGUUUUAGAGCUAGAAAUAGCAAGUUAAAAUAAGG | 16788 |
| | P12R20_ins5- | CUAGUCCGUUAUCAACUUGAAAAAGUGGCACCGAGUCGGUGCGUCUGUCUGCCUA | |
| | nick | UUUAGUGCACCGAUUUUGA | |
| RNACS5634 | TRAC4_ | GAGAAUCAAAAUCGGUGAAUGUUUUAGAGCUAGAAAUAGCAAGUUAAAAUAAGG | 16789 |
| | P11R20_ins5- | CUAGUCCGUUAUCAACUUGAAAAAGUGGCACCGAGUCGGUGCGUCUGUCUGCCUA | |
| | nick | UUUAGUGCACCGAUUUUG | |
| RNACS5635 | TRAC4_ | GAGAAUCAAAAUCGGUGAAUGUUUUAGAGCUAGAAAUAGCAAGUUAAAAUAAGG | 16790 |
| | P10R20_ins5- | CUAGUCCGUUAUCAACUUGAAAAAGUGGCACCGAGUCGGUGCGUCUGUCUGCCUA | |
| | nick | UUUAGUGCACCGAUUUU | |
| RNACS5636 | TRAC4_ | GAGAAUCAAAAUCGGUGAAUGUUUUAGAGCUAGAAAUAGCAAGUUAAAAUAAGG | 16791 |
| | P9R20_ins5- | CUAGUCCGUUAUCAACUUGAAAAAGUGGCACCGAGUCGGUGCGUCUGUCUGCCUA | |
| | nick | UUUAGUGCACCGAUUU | |
| RNACS5637 | TRAC4_ | GAGAAUCAAAAUCGGUGAAUGUUUUAGAGCUAGAAAUAGCAAGUUAAAAUAAGG | 16792 |
| | P8R20_ins5- | CUAGUCCGUUAUCAACUUGAAAAAGUGGCACCGAGUCGGUGCGUCUGUCUGCCUA | |
| | nick | UUUAGUGCACCGAUU | |
| RNACS5638 | TRAC4_ | GAGAAUCAAAAUCGGUGAAUGUUUUAGAGCUAGAAAUAGCAAGUUAAAAUAAGG | 16793 |
| | P13R22_ins5- | CUAGUCCGUUAUCAACUUGAAAAAGUGGCACCGAGUCGGUGCAAGUCUGUCUGCC | |
| | nick | UAUUUAGUGCACCGAUUUUGAU | |
| RNACS5639 | TRAC4_ | GAGAAUCAAAAUCGGUGAAUGUUUUAGAGCUAGAAAUAGCAAGUUAAAAUAAGG | 16794 |
| | P12R22_ins5- | CUAGUCCGUUAUCAACUUGAAAAAGUGGCACCGAGUCGGUGCAAGUCUGUCUGCC | |
| | nick | UAUUUAGUGCACCGAUUUUGA | |
| RNACS5640 | TRAC4_ | GAGAAUCAAAAUCGGUGAAUGUUUUAGAGCUAGAAAUAGCAAGUUAAAAUAAGG | 16795 |
| | P11R22_ins5- | CUAGUCCGUUAUCAACUUGAAAAAGUGGCACCGAGUCGGUGCAAGUCUGUCUGCC | |
| | nick | UAUUUAGUGCACCGAUUUUG | |
| RNACS5641 | TRAC4_ | GAGAAUCAAAAUCGGUGAAUGUUUUAGAGCUAGAAAUAGCAAGUUAAAAUAAGG | 16796 |
| | P10R22_ins5- | CUAGUCCGUUAUCAACUUGAAAAAGUGGCACCGAGUCGGUGCAAGUCUGUCUGCC | |
| | nick | UAUUUAGUGCACCGAUUUU | |
| RNACS5642 | TRAC4_ | GAGAAUCAAAAUCGGUGAAUGUUUUAGAGCUAGAAAUAGCAAGUUAAAAUAAGG | 16797 |
| | P9R22_ins5- | CUAGUCCGUUAUCAACUUGAAAAAGUGGCACCGAGUCGGUGCAAGUCUGUCUGCC | |
| | nick | UAUUUAGUGCACCGAUUU | |
| RNACS5643 | TRAC4_ | GAGAAUCAAAAUCGGUGAAUGUUUUAGAGCUAGAAAUAGCAAGUUAAAAUAAGG | 16798 |
| | P8R22_ins5- | CUAGUCCGUUAUCAACUUGAAAAAGUGGCACCGAGUCGGUGCAAGUCUGUCUGCC | |
| | nick | UAUUUAGUGCACCGAUU | |
| RNACS5644 | TRAC4_ | GAGAAUCAAAAUCGGUGAAUGUUUUAGAGCUAGAAAUAGCAAGUUAAAAUAAGG | 16799 |
| | P13R12_ins7- | CUAGUCCGUUAUCAACUUGAAAAAGUGGCACCGAGUCGGUGCCUAUUUAGUUGAC | |
| | nick | ACCGAUUUUGAU | |
| RNACS5645 | TRAC4_ | GAGAAUCAAAAUCGGUGAAUGUUUUAGAGCUAGAAAUAGCAAGUUAAAAUAAGG | 16800 |
| | P12R12_ins7- | CUAGUCCGUUAUCAACUUGAAAAAGUGGCACCGAGUCGGUGCCUAUUUAGUUGAC | |
| | nick | ACCGAUUUUGA | |
| RNACS5646 | TRAC4_ | GAGAAUCAAAAUCGGUGAAUGUUUUAGAGCUAGAAAUAGCAAGUUAAAAUAAGG | 16801 |
| | P11R12_ins7- | CUAGUCCGUUAUCAACUUGAAAAAGUGGCACCGAGUCGGUGCCUAUUUAGUUGAC | |
| | nick | ACCGAUUUUG | |
| RNACS5647 | TRAC4_ | GAGAAUCAAAAUCGGUGAAUGUUUUAGAGCUAGAAAUAGCAAGUUAAAAUAAGG | 16802 |
| | P10R12_ins7- | CUAGUCCGUUAUCAACUUGAAAAAGUGGCACCGAGUCGGUGCCUAUUUAGUUGAC | |
| | nick | ACCGAUUUU | |
| RNACS5648 | TRAC4_ | GAGAAUCAAAAUCGGUGAAUGUUUUAGAGCUAGAAAUAGCAAGUUAAAAUAAGG | 16803 |
| | P9R12_ins7- | CUAGUCCGUUAUCAACUUGAAAAAGUGGCACCGAGUCGGUGCCUAUUUAGUUGAC | |
| | nick | ACCGAUUU | |
| RNACS5649 | TRAC4_ | GAGAAUCAAAAUCGGUGAAUGUUUUAGAGCUAGAAAUAGCAAGUUAAAAUAAGG | 16804 |
| | P8R12_ins7- | CUAGUCCGUUAUCAACUUGAAAAAGUGGCACCGAGUCGGUGCCUAUUUAGUUGAC | |
| | nick | ACCGAUU | |
| RNACS5650 | TRAC4_ | GAGAAUCAAAAUCGGUGAAUGUUUUAGAGCUAGAAAUAGCAAGUUAAAAUAAGG | 16805 |
| | P13R14_ins7- | CUAGUCCGUUAUCAACUUGAAAAAGUGGCACCGAGUCGGUGCGCCUAUUUAGUUG | |
| | nick | ACACCGAUUUUGAU | |
| RNACS5651 | TRAC4_ | GAGAAUCAAAAUCGGUGAAUGUUUUAGAGCUAGAAAUAGCAAGUUAAAAUAAGG | 16806 |
| | P12R14_ins7- | CUAGUCCGUUAUCAACUUGAAAAAGUGGCACCGAGUCGGUGCGCCUAUUUAGUUG | |
| | nick | ACACCGAUUUUGA | |
| RNACS5652 | TRAC4_ | GAGAAUCAAAAUCGGUGAAUGUUUUAGAGCUAGAAAUAGCAAGUUAAAAUAAGG | 16807 |
| | P11R14_ins7- | CUAGUCCGUUAUCAACUUGAAAAAGUGGCACCGAGUCGGUGCGCCUAUUUAGUUG | |
| | nick | ACACCGAUUUUG | |
| RNACS5653 | TRAC4_ | GAGAAUCAAAAUCGGUGAAUGUUUUAGAGCUAGAAAUAGCAAGUUAAAAUAAGG | 16808 |
| | P10R14_ins7- | CUAGUCCGUUAUCAACUUGAAAAAGUGGCACCGAGUCGGUGCGCCUAUUUAGUUG | |
| | nick | ACACCGAUUUU | |
| RNACS5654 | TRAC4_ | GAGAAUCAAAAUCGGUGAAUGUUUUAGAGCUAGAAAUAGCAAGUUAAAAUAAGG | 16809 |
| | P9R14_ins7- | CUAGUCCGUUAUCAACUUGAAAAAGUGGCACCGAGUCGGUGCGCCUAUUUAGUUG | |
| | nick | ACACCGAUUU | |
| RNACS5655 | TRAC4_ | GAGAAUCAAAAUCGGUGAAUGUUUUAGAGCUAGAAAUAGCAAGUUAAAAUAAGG | 16810 |
| | P8R14_ins7- | CUAGUCCGUUAUCAACUUGAAAAAGUGGCACCGAGUCGGUGCGCCUAUUUAGUUG | |
| | nick | ACACCGAUU | |
| RNACS5656 | TRAC4_ | GAGAAUCAAAAUCGGUGAAUGUUUUAGAGCUAGAAAUAGCAAGUUAAAAUAAGG | 16811 |
| | P13R16_ins7- | CUAGUCCGUUAUCAACUUGAAAAAGUGGCACCGAGUCGGUGCCUGCCUAUUUAGU | |
| | nick | UGACACCGAUUUUGAU | |
| RNACS5657 | TRAC4_ | GAGAAUCAAAAUCGGUGAAUGUUUUAGAGCUAGAAAUAGCAAGUUAAAAUAAGG | 16812 |
| | P12R16_ins7- | CUAGUCCGUUAUCAACUUGAAAAAGUGGCACCGAGUCGGUGCCUGCCUAUUUAGU | |
| | nick | UGACACCGAUUUUGA | |
| RNACS5658 | TRAC4_ | GAGAAUCAAAAUCGGUGAAUGUUUUAGAGCUAGAAAUAGCAAGUUAAAAUAAGG | 16813 |
| | P11R16_ins7- | CUAGUCCGUUAUCAACUUGAAAAAGUGGCACCGAGUCGGUGCCUGCCUAUUUAGU | |
| | nick | UGACACCGAUUUUG | |
| RNACS5659 | TRAC4_ | GAGAAUCAAAAUCGGUGAAUGUUUUAGAGCUAGAAAUAGCAAGUUAAAAUAAGG | 16814 |
| | P10R16_ins7- | CUAGUCCGUUAUCAACUUGAAAAAGUGGCACCGAGUCGGUGCCUGCCUAUUUAGU | |
| | nick | UGACACCGAUUUU | |
| RNACS5660 | TRAC4_ | GAGAAUCAAAAUCGGUGAAUGUUUUAGAGCUAGAAAUAGCAAGUUAAAAUAAGG | 16815 |
| | P9R16_ins7- | CUAGUCCGUUAUCAACUUGAAAAAGUGGCACCGAGUCGGUGCCUGCCUAUUUAGU | |
| | nick | UGACACCGAUUU | |
| RNACS5661 | TRAC4_ | GAGAAUCAAAAUCGGUGAAUGUUUUAGAGCUAGAAAUAGCAAGUUAAAAUAAGG | 16816 |
| | P8R16_ins7- | CUAGUCCGUUAUCAACUUGAAAAAGUGGCACCGAGUCGGUGCCUGCCUAUUUAGU | |
| | nick | UGACACCGAUU | |
| RNACS5662 | TRAC4_ | GAGAAUCAAAAUCGGUGAAUGUUUUAGAGCUAGAAAUAGCAAGUUAAAAUAAGG | 16817 |
| | P13R18_ins7- | CUAGUCCGUUAUCAACUUGAAAAAGUGGCACCGAGUCGGUGCGUCUGCCUAUUUA | |
| | nick | GUUGACACCGAUUUUGAU | |
| RNACS5663 | TRAC4_ | GAGAAUCAAAAUCGGUGAAUGUUUUAGAGCUAGAAAUAGCAAGUUAAAAUAAGG | 16818 |
| | P12R18_ins7- | CUAGUCCGUUAUCAACUUGAAAAAGUGGCACCGAGUCGGUGCGUCUGCCUAUUUA | |
| | nick | GUUGACACCGAUUUUGA | |
| RNACS5664 | TRAC4_ | GAGAAUCAAAAUCGGUGAAUGUUUUAGAGCUAGAAAUAGCAAGUUAAAAUAAGG | 16819 |
| | P11R18_ins7- | CUAGUCCGUUAUCAACUUGAAAAAGUGGCACCGAGUCGGUGCGUCUGCCUAUUUA | |
| | nick | GUUGACACCGAUUUUG | |
| RNACS5665 | TRAC4_ | GAGAAUCAAAAUCGGUGAAUGUUUUAGAGCUAGAAAUAGCAAGUUAAAAUAAGG | 16820 |
| | P10R18_ins7- | CUAGUCCGUUAUCAACUUGAAAAAGUGGCACCGAGUCGGUGCGUCUGCCUAUUUA | |
| | nick | GUUGACACCGAUUUU | |
| RNACS5666 | TRAC4_ | GAGAAUCAAAAUCGGUGAAUGUUUUAGAGCUAGAAAUAGCAAGUUAAAAUAAGG | 16821 |
| | P9R18_ins7- | CUAGUCCGUUAUCAACUUGAAAAAGUGGCACCGAGUCGGUGCGUCUGCCUAUUUA | |
| | nick | GUUGACACCGAUUU | |
| RNACS5667 | TRAC4_ | GAGAAUCAAAAUCGGUGAAUGUUUUAGAGCUAGAAAUAGCAAGUUAAAAUAAGG | 16822 |
| | P8R18_ins7- | CUAGUCCGUUAUCAACUUGAAAAAGUGGCACCGAGUCGGUGCGUCUGCCUAUUUA | |
| | nick | GUUGACACCGAUU | |
| RNACS5668 | TRAC4_ | GAGAAUCAAAAUCGGUGAAUGUUUUAGAGCUAGAAAUAGCAAGUUAAAAUAAGG | 16823 |
| | P13R20_ins7- | CUAGUCCGUUAUCAACUUGAAAAAGUGGCACCGAGUCGGUGCCUGUCUGCCUAUU | |
| | nick | UAGUUGACACCGAUUUUGAU | |
| RNACS5669 | TRAC4_ | GAGAAUCAAAAUCGGUGAAUGUUUUAGAGCUAGAAAUAGCAAGUUAAAAUAAGG | 16824 |
| | P12R20_ins7- | CUAGUCCGUUAUCAACUUGAAAAAGUGGCACCGAGUCGGUGCCUGUCUGCCUAUU | |
| | nick | UAGUUGACACCGAUUUUGA | |
| RNACS5670 | TRAC4_ | GAGAAUCAAAAUCGGUGAAUGUUUUAGAGCUAGAAAUAGCAAGUUAAAAUAAGG | 16825 |
| | P11R20_ins7- | CUAGUCCGUUAUCAACUUGAAAAAGUGGCACCGAGUCGGUGCCUGUCUGCCUAUU | |
| | nick | UAGUUGACACCGAUUUUG | |
| RNACS5671 | TRAC4_ | GAGAAUCAAAAUCGGUGAAUGUUUUAGAGCUAGAAAUAGCAAGUUAAAAUAAGG | 16826 |
| | P10R20_ins7- | CUAGUCCGUUAUCAACUUGAAAAAGUGGCACCGAGUCGGUGCCUGUCUGCCUAUU | |
| | nick | UAGUUGACACCGAUUUU | |
| RNACS5672 | TRAC4_ | GAGAAUCAAAAUCGGUGAAUGUUUUAGAGCUAGAAAUAGCAAGUUAAAAUAAGG | 16827 |
| | P9R20_ins7- | CUAGUCCGUUAUCAACUUGAAAAAGUGGCACCGAGUCGGUGCCUGUCUGCCUAUU | |
| | nick | UAGUUGACACCGAUUU | |
| RNACS5673 | TRAC4_ | GAGAAUCAAAAUCGGUGAAUGUUUUAGAGCUAGAAAUAGCAAGUUAAAAUAAG | 16828 |
| | P8R20_ins7- | CUAGUCCGUUAUCAACUUGAAAAAGUGGCACCGAGUCGGUGCCUGUCUGCCUAUU | |
| | nick | UAGUUGACACCGAUU | |
| RNACS5674 | TRAC4_ | GAGAAUCAAAAUCGGUGAAUGUUUUAGAGCUAGAAAUAGCAAGUUAAAAUAAGG | 16829 |
| | P13R22_ins7- | CUAGUCCGUUAUCAACUUGAAAAAGUGGCACCGAGUCGGUGCGUCUGUCUGCCUA | |
| | nick | UUUAGUUGACACCGAUUUUGAU | |
| RNACS5675 | TRAC4_ | GAGAAUCAAAAUCGGUGAAUGUUUUAGAGCUAGAAAUAGCAAGUUAAAAUAAGG | 16830 |
| | P12R22_ins7- | CUAGUCCGUUAUCAACUUGAAAAAGUGGCACCGAGUCGGUGCGUCUGUCUGCCUA | |
| | nick | UUUAGUUGACACCGAUUUUGA | |
| RNACS5676 | TRAC4_ | GAGAAUCAAAAUCGGUGAAUGUUUUAGAGCUAGAAAUAGCAAGUUAAAAUAAGG | 16831 |
| | P11R22_ins7- | CUAGUCCGUUAUCAACUUGAAAAAGUGGCACCGAGUCGGUGCGUCUGUCUGCCUA | |
| | nick | UUUAGUUGACACCGAUUUUG | |
| RNACS5677 | TRAC4_ | GAGAAUCAAAAUCGGUGAAUGUUUUAGAGCUAGAAAUAGCAAGUUAAAAUAAGG | 16832 |
| | P10R22_ins7- | CUAGUCCGUUAUCAACUUGAAAAAGUGGCACCGAGUCGGUGCGUCUGUCUGCCUA | |
| | nick | UUUAGUUGACACCGAUUUU | |
| RNACS5678 | TRAC4_ | GAGAAUCAAAAUCGGUGAAUGUUUUAGAGCUAGAAAUAGCAAGUUAAAAUAAGG | 16833 |
| | P9R22_ins7- | CUAGUCCGUUAUCAACUUGAAAAAGUGGCACCGAGUCGGUGCGUCUGUCUGCCUA | |
| | nick | UUUAGUUGACACCGAUUU | |
| RNACS5679 | TRAC4_ | GAGAAUCAAAAUCGGUGAAUGUUUUAGAGCUAGAAAUAGCAAGUUAAAAUAAGG | 16834 |
| | P8R22_ins7- | CUAGUCCGUUAUCAACUUGAAAAAGUGGCACCGAGUCGGUGCGUCUGUCUGCCUA | |
| | nick | UUUAGUUGACACCGAUU | |
| RNACS5680 | TRAC4_ | GAGAAUCAAAAUCGGUGAAUGUUUUAGAGCUAGAAAUAGCAAGUUAAAAUAAGG | 16835 |
| | P13R24_ins7- | CUAGUCCGUUAUCAACUUGAAAAAGUGGCACCGAGUCGGUGCAAGUCUGUCUGCC | |
| | nick | UAUUUAGUUGACACCGAUUUUGAU | |
| RNACS5681 | TRAC4_ | GAGAAUCAAAAUCGGUGAAUGUUUUAGAGCUAGAAAUAGCAAGUUAAAAUAAGG | 16836 |
| | P12R24_ins7- | CUAGUCCGUUAUCAACUUGAAAAAGUGGCACCGAGUCGGUGCAAGUCUGUCUGCC | |
| | nick | UAUUUAGUUGACACCGAUUUUGA | |
| RNACS5682 | TRAC4_ | GAGAAUCAAAAUCGGUGAAUGUUUUAGAGCUAGAAAUAGCAAGUUAAAAUAAGG | 16837 |
| | P11R24_ins7- | CUAGUCCGUUAUCAACUUGAAAAAGUGGCACCGAGUCGGUGCAAGUCUGUCUGCC | |
| | nick | UAUUUAGUUGACACCGAUUUUG | |
| RNACS5683 | TRAC4_ | GAGAAUCAAAAUCGGUGAAUGUUUUAGAGCUAGAAAUAGCAAGUUAAAAUAAGG | 16838 |
| | P10R24_ins7- | CUAGUCCGUUAUCAACUUGAAAAAGUGGCACCGAGUCGGUGCAAGUCUGUCUGCC | |
| | nick | UAUUUAGUUGACACCGAUUUU | |
| RNACS5684 | TRAC4_ | GAGAAUCAAAAUCGGUGAAUGUUUUAGAGCUAGAAAUAGCAAGUUAAAAUAAGG | 16839 |
| | P9R24_ins7- | CUAGUCCGUUAUCAACUUGAAAAAGUGGCACCGAGUCGGUGCAAGUCUGUCUGCC | |
| | nick | UAUUUAGUUGACACCGAUUU | |
| RNACS5685 | TRAC4_ | GAGAAUCAAAAUCGGUGAAUGUUUUAGAGCUAGAAAUAGCAAGUUAAAAUAAGG | 16840 |
| | P8R24_ins7- | CUAGUCCGUUAUCAACUUGAAAAAGUGGCACCGAGUCGGUGCAAGUCUGUCUGCC | |
| | nick | UAUUUAGUUGACACCGAUU | |
| RNACS5686 | TRAC4_ | GAGAAUCAAAAUCGGUGAAUGUUUUAGAGCUAGAAAUAGCAAGUUAAAAUAAGG | 16841 |
| | P13R5_del5- | CUAGUCCGUUAUCAACUUGAAAAAGUGGCACCGAGUCGGUGCUCUGCCACCGAUU | |
| | nick | UUGAU | |
| RNACS5687 | TRAC4_ | GAGAAUCAAAAUCGGUGAAUGUUUUAGAGCUAGAAAUAGCAAGUUAAAAUAAGG | 16842 |
| | P12R5 de15- | CUAGUCCGUUAUCAACUUGAAAAAGUGGCACCGAGUCGGUGCUCUGCCACCGAUU | |
| | nick | UUGA | |
| RNACS5688 | TRAC4_ | GAGAAUCAAAAUCGGUGAAUGUUUUAGAGCUAGAAAUAGCAAGUUAAAAUAAGG | 16843 |
| | P11R5_del5- | CUAGUCCGUUAUCAACUUGAAAAAGUGGCACCGAGUCGGUGCUCUGCCACCGAUU | |
| | nick | UUG | |
| RNACS5689 | TRAC4_ | GAGAAUCAAAAUCGGUGAAUGUUUUAGAGCUAGAAAUAGCAAGUUAAAAUAAGG | 16844 |
| | P10R5_del5- | CUAGUCCGUUAUCAACUUGAAAAAGUGGCACCGAGUCGGUGCUCUGCCACCGAUU | |
| | nick | UU | |
| RNACS5690 | TRAC4_ | GAGAAUCAAAAUCGGUGAAUGUUUUAGAGCUAGAAAUAGCAAGUUAAAAUAAGG | 16845 |
| | P9R5_del5- | CUAGUCCGUUAUCAACUUGAAAAAGUGGCACCGAGUCGGUGCUCUGCCACCGAUU | |
| | nick | U | |
| RNACS5691 | TRAC4_ | GAGAAUCAAAAUCGGUGAAUGUUUUAGAGCUAGAAAUAGCAAGUUAAAAUAAGG | 16846 |
| | P8R5_del5- | CUAGUCCGUUAUCAACUUGAAAAAGUGGCACCGAGUCGGUGCUCUGCCACCGAUU | |
| | nick | | |
| RNACS5692 | TRAC4_ | GAGAAUCAAAAUCGGUGAAUGUUUUAGAGCUAGAAAUAGCAAGUUAAAAUAAGG | 16847 |
| | P13R7_del5- | CUAGUCCGUUAUCAACUUGAAAAAGUGGCACCGAGUCGGUGCUGUCUGCCACCGA | |
| | nick | UUUUGAU | |
| RNACS5693 | TRAC4_ | GAGAAUCAAAAUCGGUGAAUGUUUUAGAGCUAGAAAUAGCAAGUUAAAAUAAGG | 16848 |
| | P12R7_del5- | CUAGUCCGUUAUCAACUUGAAAAAGUGGCACCGAGUCGGUGCUGUCUGCCACCGA | |
| | nick | UUUUGA | |
| RNACS5694 | TRAC4_ | GAGAAUCAAAAUCGGUGAAUGUUUUAGAGCUAGAAAUAGCAAGUUAAAAUAAGG | 16849 |
| | P11R7 de15- | CUAGUCCGUUAUCAACUUGAAAAAGUGGCACCGAGUCGGUGCUGUCUGCCACCGA | |
| | nick | UUUUG | |
| RNACS5695 | TRAC4_ | CUAGUCCGUUAUCAACUUGAAAAAGUGGCACCGAGUCGGUGCUGUCUGCCACCGA | 16850 |
| | P10R7_del5- | GAGAAUCAAAAUCGGUGAAUGUUUUAGAGCUAGAAAUAGCAAGUUAAAAUAAGG | |
| | nick | UUUU | |
| RNACS5696 | TRAC4_ | GAGAAUCAAAAUCGGUGAAUGUUUUAGAGCUAGAAAUAGCAAGUUAAAAUAAGG | 16851 |
| | P9R7_del5- | CUAGUCCGUUAUCAACUUGAAAAAGUGGCACCGAGUCGGUGCUGUCUGCCACCGA | |
| | nick | UUU | |
| RNACS5697 | TRAC4_ | GAGAAUCAAAAUCGGUGAAUGUUUUAGAGCUAGAAAUAGCAAGUUAAAAUAAGG | 16852 |
| | P8R7_del5- | CUAGUCCGUUAUCAACUUGAAAAAGUGGCACCGAGUCGGUGCUGUCUGCCACCGA | |
| | nick | UU | |
| RNACS5698 | TRAC4_ | GAGAAUCAAAAUCGGUGAAUGUUUUAGAGCUAGAAAUAGCAAGUUAAAAUAAGG | 16853 |
| | P13R9_del5- | CUAGUCCGUUAUCAACUUGAAAAAGUGGCACCGAGUCGGUGCUCUGUCUGCCACC | |
| | nick | GAUUUUGAU | |
| RNACS5699 | TRAC4_ | GAGAAUCAAAAUCGGUGAAUGUUUUAGAGCUAGAAAUAGCAAGUUAAAAUAAGG | 16854 |
| | P12R9_del5- | CUAGUCCGUUAUCAACUUGAAAAAGUGGCACCGAGUCGGUGCUCUGUCUGCCACC | |
| | nick | GAUUUUGA | |
| RNACS5700 | TRAC4_ | GAGAAUCAAAAUCGGUGAAUGUUUUAGAGCUAGAAAUAGCAAGUUAAAAUAAGG | 16855 |
| | P11R9_del5- | CUAGUCCGUUAUCAACUUGAAAAAGUGGCACCGAGUCGGUGCUCUGUCUGCCACC | |
| | nick | GAUUUUG | |
| RNACS5701 | TRAC4_ | GAGAAUCAAAAUCGGUGAAUGUUUUAGAGCUAGAAAUAGCAAGUUAAAAUAAGG | 16856 |
| | P10R9 de15- | CUAGUCCGUUAUCAACUUGAAAAAGUGGCACCGAGUCGGUGCUCUGUCUGCCACC | |
| | nick | GAUUUU | |
| RNACS5702 | TRAC4_ | GAGAAUCAAAAUCGGUGAAUGUUUUAGAGCUAGAAAUAGCAAGUUAAAAUAAGG | 16857 |
| | P9R9_del5- | CUAGUCCGUUAUCAACUUGAAAAAGUGGCACCGAGUCGGUGCUCUGUCUGCCACC | |
| | nick | GAUUU | |
| RNACS5703 | TRAC4_ | GAGAAUCAAAAUCGGUGAAUGUUUUAGAGCUAGAAAUAGCAAGUUAAAAUAAGG | 16858 |
| | P8R9_del5- | CUAGUCCGUUAUCAACUUGAAAAAGUGGCACCGAGUCGGUGCUCUGUCUGCCACC | |
| | nick | GAUU | |
| RNACS5704 | TRAC4_ | GAGAAUCAAAAUCGGUGAAUGUUUUAGAGCUAGAAAUAGCAAGUUAAAAUAAGG | 16859 |
| | P13R11_del5- | CUAGUCCGUUAUCAACUUGAAAAAGUGGCACCGAGUCGGUGCAGUCUGUCUGCCA | |
| | nick | CCGAUUUUGAU | |
| RNACS5705 | TRAC4_ | GAGAAUCAAAAUCGGUGAAUGUUUUAGAGCUAGAAAUAGCAAGUUAAAAUAAGG | 16860 |
| | P12R11_del5- | CUAGUCCGUUAUCAACUUGAAAAAGUGGCACCGAGUCGGUGCAGUCUGUCUGCCA | |
| | nick | CCGAUUUUGA | |
| RNACS5706 | TRAC4_ | GAGAAUCAAAAUCGGUGAAUGUUUUAGAGCUAGAAAUAGCAAGUUAAAAUAAGG | 16861 |
| | P11R11_del5- | CUAGUCCGUUAUCAACUUGAAAAAGUGGCACCGAGUCGGUGCAGUCUGUCUGCCA | |
| | nick | CCGAUUUUG | |
| RNACS5707 | TRAC4_ | GAGAAUCAAAAUCGGUGAAUGUUUUAGAGCUAGAAAUAGCAAGUUAAAAUAAGG | 16862 |
| | P10R11_del5- | CUAGUCCGUUAUCAACUUGAAAAAGUGGCACCGAGUCGGUGCAGUCUGUCUGCCA | |
| | nick | CCGAUUUU | |
| RNACS5708 | TRAC4_ | GAGAAUCAAAAUCGGUGAAUGUUUUAGAGCUAGAAAUAGCAAGUUAAAAUAAGG | 16863 |
| | P9R11 de15- | CUAGUCCGUUAUCAACUUGAAAAAGUGGCACCGAGUCGGUGCAGUCUGUCUGCCA | |
| | nick | CCGAUUU | |
| RNACS5709 | TRAC4_ | GAGAAUCAAAAUCGGUGAAUGUUUUAGAGCUAGAAAUAGCAAGUUAAAAUAAGG | 16864 |
| | P8R11 de15- | CUAGUCCGUUAUCAACUUGAAAAAGUGGCACCGAGUCGGUGCAGUCUGUCUGCCA | |
| | nick | CCGAUU | |
| RNACS5710 | TRAC4_ | GAGAAUCAAAAUCGGUGAAUGUUUUAGAGCUAGAAAUAGCAAGUUAAAAUAAGG | 16865 |
| | P13R13_del5- | CUAGUCCGUUAUCAACUUGAAAAAGUGGCACCGAGUCGGUGCCAAGUCUGUCUGC | |
| | nick | CACCGAUUUUGAU | |
| RNACS5711 | TRAC4_ | GAGAAUCAAAAUCGGUGAAUGUUUUAGAGCUAGAAAUAGCAAGUUAAAAUAAGG | 16866 |
| | P12R13_del5- | CUAGUCCGUUAUCAACUUGAAAAAGUGGCACCGAGUCGGUGCCAAGUCUGUCUGC | |
| | nick | CACCGAUUUUGA | |
| RNACS5712 | TRAC4_ | GAGAAUCAAAAUCGGUGAAUGUUUUAGAGCUAGAAAUAGCAAGUUAAAAUAAGG | 16867 |
| | P11R13_del5- | CUAGUCCGUUAUCAACUUGAAAAAGUGGCACCGAGUCGGUGCCAAGUCUGUCUGC | |
| | nick | CACCGAUUUUG | |
| RNACS5713 | TRAC4_ | GAGAAUCAAAAUCGGUGAAUGUUUUAGAGCUAGAAAUAGCAAGUUAAAAUAAGG | 16868 |
| | P10R13_del5- | CUAGUCCGUUAUCAACUUGAAAAAGUGGCACCGAGUCGGUGCCAAGUCUGUCUGC | |
| | nick | CACCGAUUUU | |
| RNACS5714 | TRAC4_ | GAGAAUCAAAAUCGGUGAAUGUUUUAGAGCUAGAAAUAGCAAGUUAAAAUAAGG | 16869 |
| | P9R13_del5- | CUAGUCCGUUAUCAACUUGAAAAAGUGGCACCGAGUCGGUGCCAAGUCUGUCUGC | |
| | nick | CACCGAUUU | |
| RNACS5715 | TRAC4_ | GAGAAUCAAAAUCGGUGAAUGUUUUAGAGCUAGAAAUAGCAAGUUAAAAUAAGG | 16870 |
| | P8R13_del5- | CUAGUCCGUUAUCAACUUGAAAAAGUGGCACCGAGUCGGUGCCAAGUCUGUCUGC | |
| | nick | CACCGAUU | |
| RNACS5716 | TRAC4_ | GAGAAUCAAAAUCGGUGAAUGUUUUAGAGCUAGAAAUAGCAAGUUAAAAUAAGG | 16871 |
| | P13R15_del5- | CUAGUCCGUUAUCAACUUGAAAAAGUGGCACCGAGUCGGUGCGACAAGUCUGUCU | |
| | nick | GCCACCGAUUUUGAU | |
| RNACS5717 | TRAC4_ | GAGAAUCAAAAUCGGUGAAUGUUUUAGAGCUAGAAAUAGCAAGUUAAAAUAAGG | 16872 |
| | P12R15_del5- | CUAGUCCGUUAUCAACUUGAAAAAGUGGCACCGAGUCGGUGCGACAAGUCUGUCU | |
| | nick | GCCACCGAUUUUGA | |
| RNACS5718 | TRAC4_ | GAGAAUCAAAAUCGGUGAAUGUUUUAGAGCUAGAAAUAGCAAGUUAAAAUAAGG | 16873 |
| | P11R15_del5- | CUAGUCCGUUAUCAACUUGAAAAAGUGGCACCGAGUCGGUGCGACAAGUCUGUCU | |
| | nick | GCCACCGAUUUUG | |
| RNACS5719 | TRAC4_ | GAGAAUCAAAAUCGGUGAAUGUUUUAGAGCUAGAAAUAGCAAGUUAAAAUAAGG | 16874 |
| | P10R15_del5- | CUAGUCCGUUAUCAACUUGAAAAAGUGGCACCGAGUCGGUGCGACAAGUCUGUCU | |
| | nick | GCCACCGAUUUU | |
| RNACS5720 | TRAC4_ | GAGAAUCAAAAUCGGUGAAUGUUUUAGAGCUAGAAAUAGCAAGUUAAAAUAAGG | 16875 |
| | P9R15_del5- | CUAGUCCGUUAUCAACUUGAAAAAGUGGCACCGAGUCGGUGCGACAAGUCUGUCU | |
| | nick | GCCACCGAUUU | |
| RNACS5721 | TRAC4_ | GAGAAUCAAAAUCGGUGAAUGUUUUAGAGCUAGAAAUAGCAAGUUAAAAUAAGG | 16876 |
| | P8R15_del5- | CUAGUCCGUUAUCAACUUGAAAAAGUGGCACCGAGUCGGUGCGACAAGUCUGUCU | |
| | nick | GCCACCGAUU | |
| RNACS5722 | TRAC4_ | GAGAAUCAAAAUCGGUGAAUGUUUUAGAGCUAGAAAUAGCAAGUUAAAAUAAGG | 16877 |
| | P13R17_del5- | CUAGUCCGUUAUCAACUUGAAAAAGUGGCACCGAGUCGGUGCGUGACAAGUCUGU | |
| | nick | CUGCCACCGAUUUUGAU | |
| RNACS5723 | TRAC4_ | GAGAAUCAAAAUCGGUGAAUGUUUUAGAGCUAGAAAUAGCAAGUUAAAAUAAGG | 16878 |
| | P12R17_del5- | CUAGUCCGUUAUCAACUUGAAAAAGUGGCACCGAGUCGGUGCGUGACAAGUCUGU | |
| | nick | CUGCCACCGAUUUUGA | |
| RNACS5724 | TRAC4_ | GAGAAUCAAAAUCGGUGAAUGUUUUAGAGCUAGAAAUAGCAAGUUAAAAUAAGG | 16879 |
| | P11R17_del5- | CUAGUCCGUUAUCAACUUGAAAAAGUGGCACCGAGUCGGUGCGUGACAAGUCUGU | |
| | nick | CUGCCACCGAUUUUG | |
| RNACS5725 | TRAC4_ | GAGAAUCAAAAUCGGUGAAUGUUUUAGAGCUAGAAAUAGCAAGUUAAAAUAAGG | 16880 |
| | P10R17_del5- | CUAGUCCGUUAUCAACUUGAAAAAGUGGCACCGAGUCGGUGCGUGACAAGUCUGU | |
| | nick | CUGCCACCGAUUUU | |
| RNACS5726 | TRAC4_ | GAGAAUCAAAAUCGGUGAAUGUUUUAGAGCUAGAAAUAGCAAGUUAAAAUAAGG | 16881 |
| | P9R17_del5- | CUAGUCCGUUAUCAACUUGAAAAAGUGGCACCGAGUCGGUGCGUGACAAGUCUGU | |
| | nick | CUGCCACCGAUUU | |
| RNACS5727 | TRAC4_ | GAGAAUCAAAAUCGGUGAAUGUUUUAGAGCUAGAAAUAGCAAGUUAAAAUAAGG | 16882 |
| | P8R17_del5- | CUAGUCCGUUAUCAACUUGAAAAAGUGGCACCGAGUCGGUGCGUGACAAGUCUGU | |
| | nick | CUGCCACCGAUU | |
| RNACS5728 | TRAC4_ | GAGAAUCAAAAUCGGUGAAUGUUUUAGAGCUAGAAAUAGCAAGUUAAAAUAAGG | 16883 |
| | P13R5_del7- | CUAGUCCGUUAUCAACUUGAAAAAGUGGCACCGAGUCGGUGCUGUCUCACCGAUU | |
| | nick | UUGAU | |
| RNACS5729 | TRAC4_ | GAGAAUCAAAAUCGGUGAAUGUUUUAGAGCUAGAAAUAGCAAGUUAAAAUAAGG | 16884 |
| | P12R5_del7- | CUAGUCCGUUAUCAACUUGAAAAAGUGGCACCGAGUCGGUGCUGUCUCACCGAUU | |
| | nick | UUGA | |
| RNACS5730 | TRAC4_ | GAGAAUCAAAAUCGGUGAAUGUUUUAGAGCUAGAAAUAGCAAGUUAAAAUAAGG | 16885 |
| | P11R5_del7- | CUAGUCCGUUAUCAACUUGAAAAAGUGGCACCGAGUCGGUGCUGUCUCACCGAUU | |
| | nick | UUG | |
| RNACS5731 | TRAC4_ | GAGAAUCAAAAUCGGUGAAUGUUUUAGAGCUAGAAAUAGCAAGUUAAAAUAAGG | 16886 |
| | P10R5_del7- | CUAGUCCGUUAUCAACUUGAAAAAGUGGCACCGAGUCGGUGCUGUCUCACCGAUU | |
| | nick | UU | |
| RNACS5732 | TRAC4_ | GAGAAUCAAAAUCGGUGAAUGUUUUAGAGCUAGAAAUAGCAAGUUAAAAUAAGG | 16887 |
| | P9R5_del7- | CUAGUCCGUUAUCAACUUGAAAAAGUGGCACCGAGUCGGUGCUGUCUCACCGAUU | |
| | nick | U | |
| RNACS5733 | TRAC4_ | GAGAAUCAAAAUCGGUGAAUGUUUUAGAGCUAGAAAUAGCAAGUUAAAAUAAGG | 16888 |
| | P8R5_del7- | CUAGUCCGUUAUCAACUUGAAAAAGUGGCACCGAGUCGGUGCUGUCUCACCGAUU | |
| | nick | | |
| RNACS5734 | TRAC4_ | GAGAAUCAAAAUCGGUGAAUGUUUUAGAGCUAGAAAUAGCAAGUUAAAAUAAGG | 16889 |
| | P13R7_del7- | CUAGUCCGUUAUCAACUUGAAAAAGUGGCACCGAGUCGGUGCUCUGUCUCACCGA | |
| | nick | UUUUGAU | |
| RNACS5735 | TRAC4_ | GAGAAUCAAAAUCGGUGAAUGUUUUAGAGCUAGAAAUAGCAAGUUAAAAUAAGG | 16890 |
| | P12R7_del7- | CUAGUCCGUUAUCAACUUGAAAAAGUGGCACCGAGUCGGUGCUCUGUCUCACCGA | |
| | nick | UUUUGA | |
| RNACS5736 | TRAC4_ | GAGAAUCAAAAUCGGUGAAUGUUUUAGAGCUAGAAAUAGCAAGUUAAAAUAAGG | 16891 |
| | P11R7_del7- | CUAGUCCGUUAUCAACUUGAAAAAGUGGCACCGAGUCGGUGCUCUGUCUCACCGA | |
| | nick | UUUUG | |
| RNACS5737 | TRAC4_ | GAGAAUCAAAAUCGGUGAAUGUUUUAGAGCUAGAAAUAGCAAGUUAAAAUAAGG | 16892 |
| | P10R7_del7- | CUAGUCCGUUAUCAACUUGAAAAAGUGGCACCGAGUCGGUGCUCUGUCUCACCGA | |
| | nick | UUUU | |
| RNACS5738 | TRAC4_ | GAGAAUCAAAAUCGGUGAAUGUUUUAGAGCUAGAAAUAGCAAGUUAAAAUAAGG | 16893 |
| | P9R7_del7- | CUAGUCCGUUAUCAACUUGAAAAAGUGGCACCGAGUCGGUGCUCUGUCUCACCGA | |
| | nick | UUU | |
| RNACS5739 | TRAC4_ | GAGAAUCAAAAUCGGUGAAUGUUUUAGAGCUAGAAAUAGCAAGUUAAAAUAAGG | 16894 |
| | P8R7_del7- | CUAGUCCGUUAUCAACUUGAAAAAGUGGCACCGAGUCGGUGCUCUGUCUCACCGA | |
| | nick | UU | |
| RNACS5740 | TRAC4_ | GAGAAUCAAAAUCGGUGAAUGUUUUAGAGCUAGAAAUAGCAAGUUAAAAUAAGG | 16895 |
| | P13R9_del7- | CUAGUCCGUUAUCAACUUGAAAAAGUGGCACCGAGUCGGUGCAGUCUGUCUCACC | |
| | nick | GAUUUUGAU | |
| RNACS5741 | TRAC4_ | GAGAAUCAAAAUCGGUGAAUGUUUUAGAGCUAGAAAUAGCAAGUUAAAAUAAGG | 16896 |
| | P12R9_del7- | CUAGUCCGUUAUCAACUUGAAAAAGUGGCACCGAGUCGGUGCAGUCUGUCUCACC | |
| | nick | GAUUUUGA | |
| RNACS5742 | TRAC4_ | GAGAAUCAAAAUCGGUGAAUGUUUUAGAGCUAGAAAUAGCAAGUUAAAAUAAGG | 16897 |
| | P11R9_del7- | CUAGUCCGUUAUCAACUUGAAAAAGUGGCACCGAGUCGGUGCAGUCUGUCUCACC | |
| | nick | GAUUUUG | |
| RNACS5743 | TRAC4_ | GAGAAUCAAAAUCGGUGAAUGUUUUAGAGCUAGAAAUAGCAAGUUAAAAUAAGG | 16898 |
| | P10R9_del7- | CUAGUCCGUUAUCAACUUGAAAAAGUGGCACCGAGUCGGUGCAGUCUGUCUCACC | |
| | nick | GAUUUU | |
| RNACS5744 | TRAC4_ | GAGAAUCAAAAUCGGUGAAUGUUUUAGAGCUAGAAAUAGCAAGUUAAAAUAAGG | 16899 |
| | P9R9_del7- | CUAGUCCGUUAUCAACUUGAAAAAGUGGCACCGAGUCGGUGCAGUCUGUCUCACC | |
| | nick | GAUUU | |
| RNACS5745 | TRAC4_ | GAGAAUCAAAAUCGGUGAAUGUUUUAGAGCUAGAAAUAGCAAGUUAAAAUAAGG | 16900 |
| | P8R9_del7- | CUAGUCCGUUAUCAACUUGAAAAAGUGGCACCGAGUCGGUGCAGUCUGUCUCACC | |
| | nick | GAUU | |
| RNACS5746 | TRAC4_ | GAGAAUCAAAAUCGGUGAAUGUUUUAGAGCUAGAAAUAGCAAGUUAAAAUAAGG | 16901 |
| | P13R11_del7- | CUAGUCCGUUAUCAACUUGAAAAAGUGGCACCGAGUCGGUGCCAAGUCUGUCUCA | |
| | nick | CCGAUUUUGAU | |
| RNACS5747 | TRAC4_ | GAGAAUCAAAAUCGGUGAAUGUUUUAGAGCUAGAAAUAGCAAGUUAAAAUAAGG | 16902 |
| | P12R11_del7- | CUAGUCCGUUAUCAACUUGAAAAAGUGGCACCGAGUCGGUGCCAAGUCUGUCUCA | |
| | nick | CCGAUUUUGA | |
| RNACS5748 | TRAC4_ | GAGAAUCAAAAUCGGUGAAUGUUUUAGAGCUAGAAAUAGCAAGUUAAAAUAAGG | 16903 |
| | P11R11_del7- | CUAGUCCGUUAUCAACUUGAAAAAGUGGCACCGAGUCGGUGCCAAGUCUGUCUCA | |
| | nick | CCGAUUUUG | |
| RNACS5749 | TRAC4_ | GAGAAUCAAAAUCGGUGAAUGUUUUAGAGCUAGAAAUAGCAAGUUAAAAUAAGG | 16904 |
| | P10R11_del7- | CUAGUCCGUUAUCAACUUGAAAAAGUGGCACCGAGUCGGUGCCAAGUCUGUCUCA | |
| | nick | CCGAUUUU | |
| RNACS5750 | TRAC4_ | GAGAAUCAAAAUCGGUGAAUGUUUUAGAGCUAGAAAUAGCAAGUUAAAAUAAGG | 16905 |
| | P9R11_del7- | CUAGUCCGUUAUCAACUUGAAAAAGUGGCACCGAGUCGGUGCCAAGUCUGUCUCA | |
| | nick | CCGAUUU | |
| RNACS5751 | TRAC4_ | GAGAAUCAAAAUCGGUGAAUGUUUUAGAGCUAGAAAUAGCAAGUUAAAAUAAGG | 16906 |
| | P8R11_del7- | CUAGUCCGUUAUCAACUUGAAAAAGUGGCACCGAGUCGGUGCCAAGUCUGUCUCA | |
| | nick | CCGAUU | |
| RNACS5752 | TRAC4_ | GAGAAUCAAAAUCGGUGAAUGUUUUAGAGCUAGAAAUAGCAAGUUAAAAUAAGG | 16907 |
| | P13R13_del7- | CUAGUCCGUUAUCAACUUGAAAAAGUGGCACCGAGUCGGUGCGACAAGUCUGUCU | |
| | nick | CACCGAUUUUGAU | |
| RNACS5753 | TRAC4_ | GAGAAUCAAAAUCGGUGAAUGUUUUAGAGCUAGAAAUAGCAAGUUAAAAUAAGG | 16908 |
| | P12R13_del7- | CUAGUCCGUUAUCAACUUGAAAAAGUGGCACCGAGUCGGUGCGACAAGUCUGUCU | |
| | nick | CACCGAUUUUGA | |
| RNACS5754 | TRAC4_ | GAGAAUCAAAAUCGGUGAAUGUUUUAGAGCUAGAAAUAGCAAGUUAAAAUAAGG | 16909 |
| | P11R13_del7- | CUAGUCCGUUAUCAACUUGAAAAAGUGGCACCGAGUCGGUGCGACAAGUCUGUCU | |
| | nick | CACCGAUUUUG | |
| RNACS5755 | TRAC4_ | GAGAAUCAAAAUCGGUGAAUGUUUUAGAGCUAGAAAUAGCAAGUUAAAAUAAGG | 16910 |
| | P10R13_del7- | CUAGUCCGUUAUCAACUUGAAAAAGUGGCACCGAGUCGGUGCGACAAGUCUGUCU | |
| | nick | CACCGAUUUU | |
| RNACS5756 | TRAC4_ | GAGAAUCAAAAUCGGUGAAUGUUUUAGAGCUAGAAAUAGCAAGUUAAAAUAAGG | 16911 |
| | P9R13_del7- | CUAGUCCGUUAUCAACUUGAAAAAGUGGCACCGAGUCGGUGCGACAAGUCUGUCU | |
| | nick | CACCGAUUU | |
| RNACS5757 | TRAC4_ | GAGAAUCAAAAUCGGUGAAUGUUUUAGAGCUAGAAAUAGCAAGUUAAAAUAAGG | 16912 |
| | P8R13_del7- | CUAGUCCGUUAUCAACUUGAAAAAGUGGCACCGAGUCGGUGCGACAAGUCUGUCU | |
| | nick | CACCGAUU | |
| RNACS5758 | TRAC4_ | GAGAAUCAAAAUCGGUGAAUGUUUUAGAGCUAGAAAUAGCAAGUUAAAAUAAGG | 16913 |
| | P13R15_del7- | CUAGUCCGUUAUCAACUUGAAAAAGUGGCACCGAGUCGGUGCGUGACAAGUCUGU | |
| | nick | CUCACCGAUUUUGAU | |
| RNACS5759 | TRAC4_ | GAGAAUCAAAAUCGGUGAAUGUUUUAGAGCUAGAAAUAGCAAGUUAAAAUAAGG | 16914 |
| | P12R15_del7- | CUAGUCCGUUAUCAACUUGAAAAAGUGGCACCGAGUCGGUGCGUGACAAGUCUGU | |
| | nick | CUCACCGAUUUUGA | |
| RNACS5760 | TRAC4_ | GAGAAUCAAAAUCGGUGAAUGUUUUAGAGCUAGAAAUAGCAAGUUAAAAUAAGG | 16915 |
| | P11R15_del7- | CUAGUCCGUUAUCAACUUGAAAAAGUGGCACCGAGUCGGUGCGUGACAAGUCUGU | |
| | nick | CUCACCGAUUUUG | |
| RNACS5761 | TRAC4_ | GAGAAUCAAAAUCGGUGAAUGUUUUAGAGCUAGAAAUAGCAAGUUAAAAUAAGG | 16916 |
| | P10R15_del7- | CUAGUCCGUUAUCAACUUGAAAAAGUGGCACCGAGUCGGUGCGUGACAAGUCUGU | |
| | nick | CUCACCGAUUUU | |
| RNACS5762 | TRAC4_ | GAGAAUCAAAAUCGGUGAAUGUUUUAGAGCUAGAAAUAGCAAGUUAAAAUAAGG | 16917 |
| | P9R15_del7- | CUAGUCCGUUAUCAACUUGAAAAAGUGGCACCGAGUCGGUGCGUGACAAGUCUGU | |
| | nick | CUCACCGAUUU | |
| RNACS5763 | TRAC4_ | GAGAAUCAAAAUCGGUGAAUGUUUUAGAGCUAGAAAUAGCAAGUUAAAAUAAGG | 16918 |
| | P8R15_del7- | CUAGUCCGUUAUCAACUUGAAAAAGUGGCACCGAGUCGGUGCGUGACAAGUCUGU | |
| | nick | CUCACCGAUU | |
| RNACS5764 | TRAC4_ | GAGAAUCAAAAUCGGUGAAUGUUUUAGAGCUAGAAAUAGCAAGUUAAAAUAAGG | 16919 |
| | P13R17_del7- | CUAGUCCGUUAUCAACUUGAAAAAGUGGCACCGAGUCGGUGCCAGUGACAAGUCU | |
| | nick | GUCUCACCGAUUUUGAU | |
| RNACS5765 | TRAC4_ | GAGAAUCAAAAUCGGUGAAUGUUUUAGAGCUAGAAAUAGCAAGUUAAAAUAAGG | 16920 |
| | P12R17_del7- | CUAGUCCGUUAUCAACUUGAAAAAGUGGCACCGAGUCGGUGCCAGUGACAAGUCU | |
| | nick | GUCUCACCGAUUUUGA | |
| RNACS5766 | TRAC4_ | GAGAAUCAAAAUCGGUGAAUGUUUUAGAGCUAGAAAUAGCAAGUUAAAAUAAGG | 16921 |
| | P11R17_del7- | CUAGUCCGUUAUCAACUUGAAAAAGUGGCACCGAGUCGGUGCCAGUGACAAGUCU | |
| | nick | GUCUCACCGAUUUUG | |
| RNACS5767 | TRAC4_ | GAGAAUCAAAAUCGGUGAAUGUUUUAGAGCUAGAAAUAGCAAGUUAAAAUAAGG | 16922 |
| | P10R17_del7- | CUAGUCCGUUAUCAACUUGAAAAAGUGGCACCGAGUCGGUGCCAGUGACAAGUCU | |
| | nick | GUCUCACCGAUUUU | |
| RNACS5768 | TRAC4_ | GAGAAUCAAAAUCGGUGAAUGUUUUAGAGCUAGAAAUAGCAAGUUAAAAUAAGG | 16923 |
| | P9R17_del7- | CUAGUCCGUUAUCAACUUGAAAAAGUGGCACCGAGUCGGUGCCAGUGACAAGUCU | |
| | nick | GUCUCACCGAUUU | |
| RNACS5769 | TRAC4_ | GAGAAUCAAAAUCGGUGAAUGUUUUAGAGCUAGAAAUAGCAAGUUAAAAUAAGG | 16924 |
| | P8R17_del7- | CUAGUCCGUUAUCAACUUGAAAAAGUGGCACCGAGUCGGUGCCAGUGACAAGUCU | |
| | nick | GUCUCACCGAUU | |
| RNACS5770 | TRAC4_ | GAGAAUCAAAAUCGGUGAAUGUUUUAGAGCUAGAAAUAGCAAGUUAAAAUAAGG | 16925 |
| | P13R13_ins5- | CUAGUCCGUUAUCAACUUGAAAAAGUGGCACCGAGUCGGUGCUGCCUUAGUGAUU | |
| | PAM | CACCGAUUUUGAU | |
| RNACS5771 | TRAC4_ | GAGAAUCAAAAUCGGUGAAUGUUUUAGAGCUAGAAAUAGCAAGUUAAAAUAAGG | 16926 |
| | P12R13_ins5- | CUAGUCCGUUAUCAACUUGAAAAAGUGGCACCGAGUCGGUGCUGCCUUAGUGAUU | |
| | PAM | CACCGAUUUUGA | |
| RNACS5772 | TRAC4_ | GAGAAUCAAAAUCGGUGAAUGUUUUAGAGCUAGAAAUAGCAAGUUAAAAUAAGG | 16927 |
| | P11R13_ins5- | CUAGUCCGUUAUCAACUUGAAAAAGUGGCACCGAGUCGGUGCUGCCUUAGUGAUU | |
| | PAM | CACCGAUUUUG | |
| RNACS5773 | TRAC4_ | GAGAAUCAAAAUCGGUGAAUGUUUUAGAGCUAGAAAUAGCAAGUUAAAAUAAGG | 16928 |
| | P10R13_ins5- | CUAGUCCGUUAUCAACUUGAAAAAGUGGCACCGAGUCGGUGCUGCCUUAGUGAUU | |
| | PAM | CACCGAUUUU | |
| RNACS5774 | TRAC4_ | GAGAAUCAAAAUCGGUGAAUGUUUUAGAGCUAGAAAUAGCAAGUUAAAAUAAGG | 16929 |
| | P9R13_ins5- | CUAGUCCGUUAUCAACUUGAAAAAGUGGCACCGAGUCGGUGCUGCCUUAGUGAUU | |
| | PAM | CACCGAUUU | |
| RNACS5775 | TRAC4_ | GAGAAUCAAAAUCGGUGAAUGUUUUAGAGCUAGAAAUAGCAAGUUAAAAUAAGG | 16930 |
| | P8R13_ins5- | CUAGUCCGUUAUCAACUUGAAAAAGUGGCACCGAGUCGGUGCUGCCUUAGUGAUU | |
| | PAM | CACCGAUU | |
| RNACS5776 | TRAC4_ | GAGAAUCAAAAUCGGUGAAUGUUUUAGAGCUAGAAAUAGCAAGUUAAAAUAAGG | 16931 |
| | P13R15_ins5- | CUAGUCCGUUAUCAACUUGAAAAAGUGGCACCGAGUCGGUGCUCUGCCUUAGUGA | |
| | PAM | UUCACCGAUUUUGAU | |
| RNACS5777 | TRAC4_ | GAGAAUCAAAAUCGGUGAAUGUUUUAGAGCUAGAAAUAGCAAGUUAAAAUAAGG | 16932 |
| | P12R15_ins5- | CUAGUCCGUUAUCAACUUGAAAAAGUGGCACCGAGUCGGUGCUCUGCCUUAGUGA | |
| | PAM | UUCACCGAUUUUGA | |
| RNACS5778 | TRAC4_ | GAGAAUCAAAAUCGGUGAAUGUUUUAGAGCUAGAAAUAGCAAGUUAAAAUAAGG | 16933 |
| | P11R15_ins5- | CUAGUCCGUUAUCAACUUGAAAAAGUGGCACCGAGUCGGUGCUCUGCCUUAGUGA | |
| | PAM | UUCACCGAUUUUG | |
| RNACS5779 | TRAC4_ | GAGAAUCAAAAUCGGUGAAUGUUUUAGAGCUAGAAAUAGCAAGUUAAAAUAAGG | 16934 |
| | P10R15_ins5- | CUAGUCCGUUAUCAACUUGAAAAAGUGGCACCGAGUCGGUGCUCUGCCUUAGUGA | |
| | PAM | UUCACCGAUUUU | |
| RNACS5780 | TRAC4_ | GAGAAUCAAAAUCGGUGAAUGUUUUAGAGCUAGAAAUAGCAAGUUAAAAUAAGG | 16935 |
| | P9R15_ins5- | CUAGUCCGUUAUCAACUUGAAAAAGUGGCACCGAGUCGGUGCUCUGCCUUAGUGA | |
| | PAM | UUCACCGAUUU | |
| RNACS5781 | TRAC4_ | GAGAAUCAAAAUCGGUGAAUGUUUUAGAGCUAGAAAUAGCAAGUUAAAAUAAGG | 16936 |
| | P8R15_ins5- | CUAGUCCGUUAUCAACUUGAAAAAGUGGCACCGAGUCGGUGCUCUGCCUUAGUGA | |
| | PAM | UUCACCGAUU | |
| RNACS5782 | TRAC4_ | GAGAAUCAAAAUCGGUGAAUGUUUUAGAGCUAGAAAUAGCAAGUUAAAAUAAGG | 16937 |
| | P13R17_ins5- | CUAGUCCGUUAUCAACUUGAAAAAGUGGCACCGAGUCGGUGCUGUCUGCCUUAGU | |
| | PAM | GAUUCACCGAUUUUGAU | |
| RNACS5783 | TRAC4_ | GAGAAUCAAAAUCGGUGAAUGUUUUAGAGCUAGAAAUAGCAAGUUAAAAUAAGG | 16938 |
| | P12R17_ins5- | CUAGUCCGUUAUCAACUUGAAAAAGUGGCACCGAGUCGGUGCUGUCUGCCUUAGU | |
| | PAM | GAUUCACCGAUUUUGA | |
| RNACS5784 | TRAC4_ | GAGAAUCAAAAUCGGUGAAUGUUUUAGAGCUAGAAAUAGCAAGUUAAAAUAAGG | 16939 |
| | P11R17_ins5- | CUAGUCCGUUAUCAACUUGAAAAAGUGGCACCGAGUCGGUGCUGUCUGCCUUAGU | |
| | PAM | GAUUCACCGAUUUUG | |
| RNACS5785 | TRAC4_ | GAGAAUCAAAAUCGGUGAAUGUUUUAGAGCUAGAAAUAGCAAGUUAAAAUAAGG | 16940 |
| | P10R17_ins5- | CUAGUCCGUUAUCAACUUGAAAAAGUGGCACCGAGUCGGUGCUGUCUGCCUUAGU | |
| | PAM | GAUUCACCGAUUUU | |
| RNACS5786 | TRAC4_ | GAGAAUCAAAAUCGGUGAAUGUUUUAGAGCUAGAAAUAGCAAGUUAAAAUAAGG | 16941 |
| | P9R17_ins5- | CUAGUCCGUUAUCAACUUGAAAAAGUGGCACCGAGUCGGUGCUGUCUGCCUUAGU | |
| | PAM | GAUUCACCGAUUU | |
| RNACS5787 | TRAC4_ | GAGAAUCAAAAUCGGUGAAUGUUUUAGAGCUAGAAAUAGCAAGUUAAAAUAAGG | 16942 |
| | P8R17_ins5- | CUAGUCCGUUAUCAACUUGAAAAAGUGGCACCGAGUCGGUGCUGUCUGCCUUAGU | |
| | PAM | GAUUCACCGAUU | |
| RNACS5788 | TRAC4_ | GAGAAUCAAAAUCGGUGAAUGUUUUAGAGCUAGAAAUAGCAAGUUAAAAUAAGG | 16943 |
| | P13R19_ins5- | CUAGUCCGUUAUCAACUUGAAAAAGUGGCACCGAGUCGGUGCUCUGUCUGCCUUA | |
| | PAM | GUGAUUCACCGAUUUUGAU | |
| RNACS5789 | TRAC4_ | GAGAAUCAAAAUCGGUGAAUGUUUUAGAGCUAGAAAUAGCAAGUUAAAAUAAGG | 16944 |
| | P12R19_ins5- | CUAGUCCGUUAUCAACUUGAAAAAGUGGCACCGAGUCGGUGCUCUGUCUGCCUUA | |
| | PAM | GUGAUUCACCGAUUUUGA | |
| RNACS5790 | TRAC4_ | GAGAAUCAAAAUCGGUGAAUGUUUUAGAGCUAGAAAUAGCAAGUUAAAAUAAGG | 16945 |
| | P11R19_ins5- | CUAGUCCGUUAUCAACUUGAAAAAGUGGCACCGAGUCGGUGCUCUGUCUGCCUUA | |
| | PAM | GUGAUUCACCGAUUUUG | |
| RNACS5791 | TRAC4_ | GAGAAUCAAAAUCGGUGAAUGUUUUAGAGCUAGAAAUAGCAAGUUAAAAUAAGG | 16946 |
| | P10R19_ins5- | CUAGUCCGUUAUCAACUUGAAAAAGUGGCACCGAGUCGGUGCUCUGUCUGCCUUA | |
| | PAM | GUGAUUCACCGAUUUU | |
| RNACS5792 | TRAC4_ | GAGAAUCAAAAUCGGUGAAUGUUUUAGAGCUAGAAAUAGCAAGUUAAAAUAAGG | 16947 |
| | P9R19_ins5- | CUAGUCCGUUAUCAACUUGAAAAAGUGGCACCGAGUCGGUGCUCUGUCUGCCUUA | |
| | PAM | GUGAUUCACCGAUUU | |
| RNACS5793 | TRAC4_ | GAGAAUCAAAAUCGGUGAAUGUUUUAGAGCUAGAAAUAGCAAGUUAAAAUAAGG | 16948 |
| | P8R19_ins5- | CUAGUCCGUUAUCAACUUGAAAAAGUGGCACCGAGUCGGUGCUCUGUCUGCCUUA | |
| | PAM | GUGAUUCACCGAUU | |
| RNACS5794 | TRAC4_ | GAGAAUCAAAAUCGGUGAAUGUUUUAGAGCUAGAAAUAGCAAGUUAAAAUAAGG | 16949 |
| | P13R21_ins5- | CUAGUCCGUUAUCAACUUGAAAAAGUGGCACCGAGUCGGUGCAGUCUGUCUGCCU | |
| | PAM | UAGUGAUUCACCGAUUUUGAU | |
| RNACS5795 | TRAC4_ | GAGAAUCAAAAUCGGUGAAUGUUUUAGAGCUAGAAAUAGCAAGUUAAAAUAAGG | 16950 |
| | P12R21_ins5- | CUAGUCCGUUAUCAACUUGAAAAAGUGGCACCGAGUCGGUGCAGUCUGUCUGCCU | |
| | PAM | UAGUGAUUCACCGAUUUUGA | |
| RNACS5796 | TRAC4_ | GAGAAUCAAAAUCGGUGAAUGUUUUAGAGCUAGAAAUAGCAAGUUAAAAUAAGG | 16951 |
| | P11R21_ins5- | CUAGUCCGUUAUCAACUUGAAAAAGUGGCACCGAGUCGGUGCAGUCUGUCUGCCU | |
| | PAM | UAGUGAUUCACCGAUUUUG | |
| RNACS5797 | TRAC4_ | GAGAAUCAAAAUCGGUGAAUGUUUUAGAGCUAGAAAUAGCAAGUUAAAAUAAGG | 16952 |
| | P10R21_ins5- | CUAGUCCGUUAUCAACUUGAAAAAGUGGCACCGAGUCGGUGCAGUCUGUCUGCCU | |
| | PAM | UAGUGAUUCACCGAUUUU | |
| RNACS5798 | TRAC4_ | GAGAAUCAAAAUCGGUGAAUGUUUUAGAGCUAGAAAUAGCAAGUUAAAAUAAGG | 16953 |
| | RP921_ins5- | CUAGUCCGUUAUCAACUUGAAAAAGUGGCACCGAGUCGGUGCAGUCUGUCUGCCU | |
| | PAM | UAGUGAUUCACCGAUUU | |
| RNACS5799 | TRAC4_ | GAGAAUCAAAAUCGGUGAAUGUUUUAGAGCUAGAAAUAGCAAGUUAAAAUAAGG | 16954 |
| | P8R21_ins5- | CUAGUCCGUUAUCAACUUGAAAAAGUGGCACCGAGUCGGUGCAGUCUGUCUGCCU | |
| | PAM | UAGUGAUUCACCGAUU | |
| RNACS5800 | TRAC4_ | GAGAAUCAAAAUCGGUGAAUGUUUUAGAGCUAGAAAUAGCAAGUUAAAAUAAGG | 16955 |
| | P13R23_ins5- | CUAGUCCGUUAUCAACUUGAAAAAGUGGCACCGAGUCGGUGCCAAGUCUGUCUGC | |
| | PAM | CUUAGUGAUUCACCGAUUUUGAU | |
| RNACS5801 | TRAC4_ | GAGAAUCAAAAUCGGUGAAUGUUUUAGAGCUAGAAAUAGCAAGUUAAAAUAAGG | 16956 |
| | P12R23_ins5- | CUAGUCCGUUAUCAACUUGAAAAAGUGGCACCGAGUCGGUGCCAAGUCUGUCUGC | |
| | PAM | CUUAGUGAUUCACCGAUUUUGA | |
| RNACS5802 | TRAC4_ | GAGAAUCAAAAUCGGUGAAUGUUUUAGAGCUAGAAAUAGCAAGUUAAAAUAAGG | 16957 |
| | P11R23_ins5- | CUAGUCCGUUAUCAACUUGAAAAAGUGGCACCGAGUCGGUGCCAAGUCUGUCUGC | |
| | PAM | CUUAGUGAUUCACCGAUUUUG | |
| RNACS5803 | TRAC4_ | GAGAAUCAAAAUCGGUGAAUGUUUUAGAGCUAGAAAUAGCAAGUUAAAAUAAGG | 16958 |
| | P10R23_ins5- | CUAGUCCGUUAUCAACUUGAAAAAGUGGCACCGAGUCGGUGCCAAGUCUGUCUGC | |
| | PAM | CUUAGUGAUUCACCGAUUUU | |
| RNACS5804 | TRAC4_ | GAGAAUCAAAAUCGGUGAAUGUUUUAGAGCUAGAAAUAGCAAGUUAAAAUAAGG | 16959 |
| | P9R23_ins5- | CUAGUCCGUUAUCAACUUGAAAAAGUGGCACCGAGUCGGUGCCAAGUCUGUCUGC | |
| | PAM | CUUAGUGAUUCACCGAUUU | |
| RNACS5805 | TRAC4_ | GAGAAUCAAAAUCGGUGAAUGUUUUAGAGCUAGAAAUAGCAAGUUAAAAUAAGG | 16960 |
| | P8R23_ins5- | CUAGUCCGUUAUCAACUUGAAAAAGUGGCACCGAGUCGGUGCCAAGUCUGUCUGC | |
| | PAM | CUUAGUGAUUCACCGAUU | |
| RNACS5806 | TRAC4_ | GAGAAUCAAAAUCGGUGAAUGUUUUAGAGCUAGAAAUAGCAAGUUAAAAUAAGG | 16961 |
| | P13R25_ins5- | CUAGUCCGUUAUCAACUUGAAAAAGUGGCACCGAGUCGGUGCGACAAGUCUGUCU | |
| | PAM | GCCUUAGUGAUUCACCGAUUUUGAU | |
| RNACS5807 | TRAC4_ | GAGAAUCAAAAUCGGUGAAUGUUUUAGAGCUAGAAAUAGCAAGUUAAAAUAAGG | 16962 |
| | P12R25_ins5- | CUAGUCCGUUAUCAACUUGAAAAAGUGGCACCGAGUCGGUGCGACAAGUCUGUCU | |
| | PAM | GCCUUAGUGAUUCACCGAUUUUGA | |
| RNACS5808 | TRAC4_ | GAGAAUCAAAAUCGGUGAAUGUUUUAGAGCUAGAAAUAGCAAGUUAAAAUAAGG | 16963 |
| | P11R25_ins5- | CUAGUCCGUUAUCAACUUGAAAAAGUGGCACCGAGUCGGUGCGACAAGUCUGUCU | |
| | PAM | GCCUUAGUGAUUCACCGAUUUUG | |
| RNACS5809 | TRAC4_ | GAGAAUCAAAAUCGGUGAAUGUUUUAGAGCUAGAAAUAGCAAGUUAAAAUAAGG | 16964 |
| | P10R25_ins5- | CUAGUCCGUUAUCAACUUGAAAAAGUGGCACCGAGUCGGUGCGACAAGUCUGUCU | |
| | PAM | GCCUUAGUGAUUCACCGAUUUU | |
| RNACS5810 | TRAC4_ | GAGAAUCAAAAUCGGUGAAUGUUUUAGAGCUAGAAAUAGCAAGUUAAAAUAAGG | 16965 |
| | P9R25_ins5- | CUAGUCCGUUAUCAACUUGAAAAAGUGGCACCGAGUCGGUGCGACAAGUCUGUCU | |
| | PAM | GCCUUAGUGAUUCACCGAUUU | |
| RNACS5811 | TRAC4_ | GAGAAUCAAAAUCGGUGAAUGUUUUAGAGCUAGAAAUAGCAAGUUAAAAUAAGG | 16966 |
| | P8R25_ins5- | CUAGUCCGUUAUCAACUUGAAAAAGUGGCACCGAGUCGGUGCGACAAGUCUGUCU | |
| | PAM | GCCUUAGUGAUUCACCGAUU | |
| RNACS5812 | TRAC4_ | GAGAAUCAAAAUCGGUGAAUGUUUUAGAGCUAGAAAUAGCAAGUUAAAAUAAGG | 16967 |
| | P13R15_ins7- | CUAGUCCGUUAUCAACUUGAAAAAGUGGCACCGAGUCGGUGCUGCCUUAGUUGAA | |
| | PAM | UUCACCGAUUUUGAU | |
| RNACS5813 | TRAC4_ | GAGAAUCAAAAUCGGUGAAUGUUUUAGAGCUAGAAAUAGCAAGUUAAAAUAAGG | 16968 |
| | P12R15_ins7- | CUAGUCCGUUAUCAACUUGAAAAAGUGGCACCGAGUCGGUGCUGCCUUAGUUGAA | |
| | PAM | UUCACCGAUUUUGA | |
| RNACS5814 | TRAC4_ | GAGAAUCAAAAUCGGUGAAUGUUUUAGAGCUAGAAAUAGCAAGUUAAAAUAAGG | 16969 |
| | P11R15_ins7- | CUAGUCCGUUAUCAACUUGAAAAAGUGGCACCGAGUCGGUGCUGCCUUAGUUGAA | |
| | PAM | UUCACCGAUUUUG | |
| RNACS5815 | TRAC4_ | GAGAAUCAAAAUCGGUGAAUGUUUUAGAGCUAGAAAUAGCAAGUUAAAAUAAGG | 16970 |
| | P10R15_ins7- | CUAGUCCGUUAUCAACUUGAAAAAGUGGCACCGAGUCGGUGCUGCCUUAGUUGAA | |
| | PAM | UUCACCGAUUUU | |
| RNACS5816 | TRAC4_ | GAGAAUCAAAAUCGGUGAAUGUUUUAGAGCUAGAAAUAGCAAGUUAAAAUAAGG | 16971 |
| | P9R15_ins7- | CUAGUCCGUUAUCAACUUGAAAAAGUGGCACCGAGUCGGUGCUGCCUUAGUUGAA | |
| | PAM | UUCACCGAUUU | |
| RNACS5817 | TRAC4_ | GAGAAUCAAAAUCGGUGAAUGUUUUAGAGCUAGAAAUAGCAAGUUAAAAUAAGG | 16972 |
| | P8R15_ins7- | CUAGUCCGUUAUCAACUUGAAAAAGUGGCACCGAGUCGGUGCUGCCUUAGUUGAA | |
| | PAM | UUCACCGAUU | |
| RNACS5818 | TRAC4_ | GAGAAUCAAAAUCGGUGAAUGUUUUAGAGCUAGAAAUAGCAAGUUAAAAUAAGG | 16973 |
| | P13R17_ins7- | CUAGUCCGUUAUCAACUUGAAAAAGUGGCACCGAGUCGGUGCUCUGCCUUAGUUG | |
| | PAM | AAUUCACCGAUUUUGAU | |
| RNACS5819 | TRAC4_ | GAGAAUCAAAAUCGGUGAAUGUUUUAGAGCUAGAAAUAGCAAGUUAAAAUAAGG | 16974 |
| | P12R17_ins7- | CUAGUCCGUUAUCAACUUGAAAAAGUGGCACCGAGUCGGUGCUCUGCCUUAGUUG | |
| | PAM | AAUUCACCGAUUUUGA | |
| RNACS5820 | TRAC4_ | GAGAAUCAAAAUCGGUGAAUGUUUUAGAGCUAGAAAUAGCAAGUUAAAAUAAGG | 16975 |
| | P11R17_ins7- | CUAGUCCGUUAUCAACUUGAAAAAGUGGCACCGAGUCGGUGCUCUGCCUUAGUUG | |
| | PAM | AAUUCACCGAUUUUG | |
| RNACS5821 | TRAC4_ | GAGAAUCAAAAUCGGUGAAUGUUUUAGAGCUAGAAAUAGCAAGUUAAAAUAAGG | 16976 |
| | P10R17_ins7- | CUAGUCCGUUAUCAACUUGAAAAAGUGGCACCGAGUCGGUGCUCUGCCUUAGUUG | |
| | PAM | AAUUCACCGAUUUU | |
| RNACS5822 | TRAC4_ | GAGAAUCAAAAUCGGUGAAUGUUUUAGAGCUAGAAAUAGCAAGUUAAAAUAAGG | 16977 |
| | P9R17_ins7- | CUAGUCCGUUAUCAACUUGAAAAAGUGGCACCGAGUCGGUGCUCUGCCUUAGUUG | |
| | PAM | AAUUCACCGAUUU | |
| RNACS5823 | TRAC4_ | GAGAAUCAAAAUCGGUGAAUGUUUUAGAGCUAGAAAUAGCAAGUUAAAAUAAGG | 16978 |
| | P8R17_ins7- | CUAGUCCGUUAUCAACUUGAAAAAGUGGCACCGAGUCGGUGCUCUGCCUUAGUUG | |
| | PAM | AAUUCACCGAUU | |
| RNACS5824 | TRAC4_ | GAGAAUCAAAAUCGGUGAAUGUUUUAGAGCUAGAAAUAGCAAGUUAAAAUAAGG | 16979 |
| | P13R19_ins7- | CUAGUCCGUUAUCAACUUGAAAAAGUGGCACCGAGUCGGUGCUGUCUGCCUUAGU | |
| | PAM | UGAAUUCACCGAUUUUGAU | |
| RNACS5825 | TRAC4_ | GAGAAUCAAAAUCGGUGAAUGUUUUAGAGCUAGAAAUAGCAAGUUAAAAUAAGG | 16980 |
| | P12R19_ins7- | CUAGUCCGUUAUCAACUUGAAAAAGUGGCACCGAGUCGGUGCUGUCUGCCUUAGU | |
| | PAM | UGAAUUCACCGAUUUUGA | |
| RNACS5826 | TRAC4_ | GAGAAUCAAAAUCGGUGAAUGUUUUAGAGCUAGAAAUAGCAAGUUAAAAUAAGG | 16981 |
| | P11R19_ins7- | CUAGUCCGUUAUCAACUUGAAAAAGUGGCACCGAGUCGGUGCUGUCUGCCUUAGU | |
| | PAM | UGAAUUCACCGAUUUUG | |
| RNACS5827 | TRAC4_ | GAGAAUCAAAAUCGGUGAAUGUUUUAGAGCUAGAAAUAGCAAGUUAAAAUAAGG | 16982 |
| | P10R19_ins7- | CUAGUCCGUUAUCAACUUGAAAAAGUGGCACCGAGUCGGUGCUGUCUGCCUUAGU | |
| | PAM | UGAAUUCACCGAUUUU | |
| RNACS5828 | TRAC4_ | GAGAAUCAAAAUCGGUGAAUGUUUUAGAGCUAGAAAUAGCAAGUUAAAAUAAGG | 16983 |
| | P9R19_ins7- | CUAGUCCGUUAUCAACUUGAAAAAGUGGCACCGAGUCGGUGCUGUCUGCCUUAGU | |
| | PAM | UGAAUUCACCGAUUU | |
| RNACS5829 | TRAC4_ | GAGAAUCAAAAUCGGUGAAUGUUUUAGAGCUAGAAAUAGCAAGUUAAAAUAAGG | 16984 |
| | P8R19_ins7- | CUAGUCCGUUAUCAACUUGAAAAAGUGGCACCGAGUCGGUGCUGUCUGCCUUAGU | |
| | PAM | UGAAUUCACCGAUU | |
| RNACS5830 | TRAC4_ | GAGAAUCAAAAUCGGUGAAUGUUUUAGAGCUAGAAAUAGCAAGUUAAAAUAAGG | 16985 |
| | P13R21_ins7- | CUAGUCCGUUAUCAACUUGAAAAAGUGGCACCGAGUCGGUGCUCUGUCUGCCUUA | |
| | PAM | GUUGAAUUCACCGAUUUUGAU | |
| RNACS5831 | TRAC4_ | GAGAAUCAAAAUCGGUGAAUGUUUUAGAGCUAGAAAUAGCAAGUUAAAAUAAGG | 16986 |
| | P12R21_ins7- | CUAGUCCGUUAUCAACUUGAAAAAGUGGCACCGAGUCGGUGCUCUGUCUGCCUUA | |
| | PAM | GUUGAAUUCACCGAUUUUGA | |
| RNACS5832 | TRAC4_ | GAGAAUCAAAAUCGGUGAAUGUUUUAGAGCUAGAAAUAGCAAGUUAAAAUAAGG | 16987 |
| | P11R21_ins7- | CUAGUCCGUUAUCAACUUGAAAAAGUGGCACCGAGUCGGUGCUCUGUCUGCCUUA | |
| | PAM | GUUGAAUUCACCGAUUUUG | |
| RNACS5833 | TRAC4_ | GAGAAUCAAAAUCGGUGAAUGUUUUAGAGCUAGAAAUAGCAAGUUAAAAUAAGG | 16988 |
| | P10R21_ins7- | CUAGUCCGUUAUCAACUUGAAAAAGUGGCACCGAGUCGGUGCUCUGUCUGCCUUA | |
| | PAM | GUUGAAUUCACCGAUUUU | |
| RNACS5834 | TRAC4_ | GAGAAUCAAAAUCGGUGAAUGUUUUAGAGCUAGAAAUAGCAAGUUAAAAUAAGG | 16989 |
| | P9R21_ins7- | CUAGUCCGUUAUCAACUUGAAAAAGUGGCACCGAGUCGGUGCUCUGUCUGCCUUA | |
| | PAM | GUUGAAUUCACCGAUUU | |
| RNACS5835 | TRAC4_ | GAGAAUCAAAAUCGGUGAAUGUUUUAGAGCUAGAAAUAGCAAGUUAAAAUAAGG | 16990 |
| | P8R21_ins7- | CUAGUCCGUUAUCAACUUGAAAAAGUGGCACCGAGUCGGUGCUCUGUCUGCCUUA | |
| | PAM | GUUGAAUUCACCGAUU | |
| RNACS5836 | TRAC4_ | GAGAAUCAAAAUCGGUGAAUGUUUUAGAGCUAGAAAUAGCAAGUUAAAAUAAGG | 16991 |
| | P13R23_ins7- | CUAGUCCGUUAUCAACUUGAAAAAGUGGCACCGAGUCGGUGCAGUCUGUCUGCCU | |
| | PAM | UAGUUGAAUUCACCGAUUUUGAU | |
| RNACS5837 | TRAC4_ | GAGAAUCAAAAUCGGUGAAUGUUUUAGAGCUAGAAAUAGCAAGUUAAAAUAAGG | 16992 |
| | P12R23_ins7- | CUAGUCCGUUAUCAACUUGAAAAAGUGGCACCGAGUCGGUGCAGUCUGUCUGCCU | |
| | PAM | UAGUUGAAUUCACCGAUUUUGA | |
| RNACS5838 | TRAC4_ | GAGAAUCAAAAUCGGUGAAUGUUUUAGAGCUAGAAAUAGCAAGUUAAAAUAAGG | 16993 |
| | P11R23_ins7- | CUAGUCCGUUAUCAACUUGAAAAAGUGGCACCGAGUCGGUGCAGUCUGUCUGCCU | |
| | PAM | UAGUUGAAUUCACCGAUUUUG | |
| RNACS5839 | TRAC4_ | GAGAAUCAAAAUCGGUGAAUGUUUUAGAGCUAGAAAUAGCAAGUUAAAAUAAGG | 16994 |
| | P10R23_ins7- | CUAGUCCGUUAUCAACUUGAAAAAGUGGCACCGAGUCGGUGCAGUCUGUCUGCCU | |
| | PAM | UAGUUGAAUUCACCGAUUUU | |
| RNACS5840 | TRAC4_ | GAGAAUCAAAAUCGGUGAAUGUUUUAGAGCUAGAAAUAGCAAGUUAAAAUAAGG | 16995 |
| | P9R23_ins7- | CUAGUCCGUUAUCAACUUGAAAAAGUGGCACCGAGUCGGUGCAGUCUGUCUGCCU | |
| | PAM | UAGUUGAAUUCACCGAUUU | |
| RNACS5841 | TRAC4_ | GAGAAUCAAAAUCGGUGAAUGUUUUAGAGCUAGAAAUAGCAAGUUAAAAUAAGG | 16996 |
| | P8R23_ins7- | CUAGUCCGUUAUCAACUUGAAAAAGUGGCACCGAGUCGGUGCAGUCUGUCUGCCU | |
| | PAM | UAGUUGAAUUCACCGAUU | |
| RNACS5842 | TRAC4_ | GAGAAUCAAAAUCGGUGAAUGUUUUAGAGCUAGAAAUAGCAAGUUAAAAUAAGG | 16997 |
| | P13R25_ins7- | CUAGUCCGUUAUCAACUUGAAAAAGUGGCACCGAGUCGGUGCCAAGUCUGUCUGC | |
| | PAM | CUUAGUUGAAUUCACCGAUUUUGAU | |
| RNACS5843 | TRAC4_ | GAGAAUCAAAAUCGGUGAAUGUUUUAGAGCUAGAAAUAGCAAGUUAAAAUAAGG | 16998 |
| | P12R25_ins7- | CUAGUCCGUUAUCAACUUGAAAAAGUGGCACCGAGUCGGUGCCAAGUCUGUCUGC | |
| | PAM | CUUAGUUGAAUUCACCGAUUUUGA | |
| RNACS5844 | TRAC4_ | GAGAAUCAAAAUCGGUGAAUGUUUUAGAGCUAGAAAUAGCAAGUUAAAAUAAGG | 16999 |
| | P11R25_ins7- | CUAGUCCGUUAUCAACUUGAAAAAGUGGCACCGAGUCGGUGCCAAGUCUGUCUGC | |
| | PAM | CUUAGUUGAAUUCACCGAUUUUG | |
| RNACS5845 | TRAC4_ | GAGAAUCAAAAUCGGUGAAUGUUUUAGAGCUAGAAAUAGCAAGUUAAAAUAAGG | 17000 |
| | P10R25_ins7- | CUAGUCCGUUAUCAACUUGAAAAAGUGGCACCGAGUCGGUGCCAAGUCUGUCUGC | |
| | PAM | CUUAGUUGAAUUCACCGAUUUU | |
| RNACS5846 | TRAC4_ | GAGAAUCAAAAUCGGUGAAUGUUUUAGAGCUAGAAAUAGCAAGUUAAAAUAAGG | 17001 |
| | P9R25_ins7- | CUAGUCCGUUAUCAACUUGAAAAAGUGGCACCGAGUCGGUGCCAAGUCUGUCUGC | |
| | PAM | CUUAGUUGAAUUCACCGAUUU | |
| RNACS5847 | TRAC4_ | GAGAAUCAAAAUCGGUGAAUGUUUUAGAGCUAGAAAUAGCAAGUUAAAAUAAGG | 17002 |
| | P8R25_ins7- | CUAGUCCGUUAUCAACUUGAAAAAGUGGCACCGAGUCGGUGCCAAGUCUGUCUGC | |
| | PAM | CUUAGUUGAAUUCACCGAUU | |
| RNACS5848 | TRAC4_ | GAGAAUCAAAAUCGGUGAAUGUUUUAGAGCUAGAAAUAGCAAGUUAAAAUAAGG | 17003 |
| | P13R27_ins7- | CUAGUCCGUUAUCAACUUGAAAAAGUGGCACCGAGUCGGUGCGACAAGUCUGUCU | |
| | PAM | GCCUUAGUUGAAUUCACCGAUUUUGAU | |
| RNACS5849 | TRAC4_ | GAGAAUCAAAAUCGGUGAAUGUUUUAGAGCUAGAAAUAGCAAGUUAAAAUAAGG | 17004 |
| | P12R27_ins7- | CUAGUCCGUUAUCAACUUGAAAAAGUGGCACCGAGUCGGUGCGACAAGUCUGUCU | |
| | PAM | GCCUUAGUUGAAUUCACCGAUUUUGA | |
| RNACS5850 | TRAC4_ | GAGAAUCAAAAUCGGUGAAUGUUUUAGAGCUAGAAAUAGCAAGUUAAAAUAAGG | 17005 |
| | P11R27_ins7- | CUAGUCCGUUAUCAACUUGAAAAAGUGGCACCGAGUCGGUGCGACAAGUCUGUCU | |
| | PAM | GCCUUAGUUGAAUUCACCGAUUUUG | |
| RNACS5851 | TRAC4_ | GAGAAUCAAAAUCGGUGAAUGUUUUAGAGCUAGAAAUAGCAAGUUAAAAUAAGG | 17006 |
| | P10R27_ins7- | CUAGUCCGUUAUCAACUUGAAAAAGUGGCACCGAGUCGGUGCGACAAGUCUGUCU | |
| | PAM | GCCUUAGUUGAAUUCACCGAUUUU | |
| RNACS5852 | TRAC4_ | GAGAAUCAAAAUCGGUGAAUGUUUUAGAGCUAGAAAUAGCAAGUUAAAAUAAGG | 17007 |
| | P9R27_ins7- | CUAGUCCGUUAUCAACUUGAAAAAGUGGCACCGAGUCGGUGCGACAAGUCUGUCU | |
| | PAM | GCCUUAGUUGAAUUCACCGAUUU | |
| RNACS5853 | TRAC4_ | GAGAAUCAAAAUCGGUGAAUGUUUUAGAGCUAGAAAUAGCAAGUUAAAAUAAGG | 17008 |
| | P8R27_ins7- | CUAGUCCGUUAUCAACUUGAAAAAGUGGCACCGAGUCGGUGCGACAAGUCUGUCU | |
| | PAM | GCCUUAGUUGAAUUCACCGAUU | |
| RNACS5854 | TRAC4_ | GAGAAUCAAAAUCGGUGAAUGUUUUAGAGCUAGAAAUAGCAAGUUAAAAUAAGG | 17009 |
| | P13R8_del5- | CUAGUCCGUUAUCAACUUGAAAAAGUGGCACCGAGUCGGUGCCUGUCAUUCACCG | |
| | PAM | AUUUUGAU | |
| RNACS5855 | TRAC4_ | GAGAAUCAAAAUCGGUGAAUGUUUUAGAGCUAGAAAUAGCAAGUUAAAAUAAGG | 17010 |
| | P12R8_del5- | CUAGUCCGUUAUCAACUUGAAAAAGUGGCACCGAGUCGGUGCCUGUCAUUCACCG | |
| | PAM | AUUUUGA | |
| RNACS5856 | TRAC4_ | GAGAAUCAAAAUCGGUGAAUGUUUUAGAGCUAGAAAUAGCAAGUUAAAAUAAGG | 17011 |
| | P11R8_del5- | CUAGUCCGUUAUCAACUUGAAAAAGUGGCACCGAGUCGGUGCCUGUCAUUCACCG | |
| | PAM | AUUUUG | |
| RNACS5857 | TRAC4_ | GAGAAUCAAAAUCGGUGAAUGUUUUAGAGCUAGAAAUAGCAAGUUAAAAUAAGG | 17012 |
| | P10R8_del5- | CUAGUCCGUUAUCAACUUGAAAAAGUGGCACCGAGUCGGUGCCUGUCAUUCACCG | |
| | PAM | AUUUU | |
| RNACS5858 | TRAC4_ | GAGAAUCAAAAUCGGUGAAUGUUUUAGAGCUAGAAAUAGCAAGUUAAAAUAAGG | 17013 |
| | P9R8_del5- | CUAGUCCGUUAUCAACUUGAAAAAGUGGCACCGAGUCGGUGCCUGUCAUUCACCG | |
| | PAM | AUUU | |
| RNACS5859 | TRAC4_ | GAGAAUCAAAAUCGGUGAAUGUUUUAGAGCUAGAAAUAGCAAGUUAAAAUAAGG | 17014 |
| | P8R8_del5- | CUAGUCCGUUAUCAACUUGAAAAAGUGGCACCGAGUCGGUGCCUGUCAUUCACCG | |
| | PAM | AUU | |
| RNACS5860 | TRAC4_ | GAGAAUCAAAAUCGGUGAAUGUUUUAGAGCUAGAAAUAGCAAGUUAAAAUAAGG | 17015 |
| | P13R10_del5- | CUAGUCCGUUAUCAACUUGAAAAAGUGGCACCGAGUCGGUGCGUCUGUCAUUCAC | |
| | PAM | CGAUUUUGAU | |
| RNACS5861 | TRAC4_ | GAGAAUCAAAAUCGGUGAAUGUUUUAGAGCUAGAAAUAGCAAGUUAAAAUAAGG | 17016 |
| | P12R10_del5- | CUAGUCCGUUAUCAACUUGAAAAAGUGGCACCGAGUCGGUGCGUCUGUCAUUCAC | |
| | PAM | CGAUUUUGA | |
| RNACS5862 | TRAC4_ | GAGAAUCAAAAUCGGUGAAUGUUUUAGAGCUAGAAAUAGCAAGUUAAAAUAAGG | 17017 |
| | P11R10_del5- | CUAGUCCGUUAUCAACUUGAAAAAGUGGCACCGAGUCGGUGCGUCUGUCAUUCAC | |
| | PAM | CGAUUUUG | |
| RNACS5863 | TRAC4_ | GAGAAUCAAAAUCGGUGAAUGUUUUAGAGCUAGAAAUAGCAAGUUAAAAUAAGG | 17018 |
| | P10R10_del5- | CUAGUCCGUUAUCAACUUGAAAAAGUGGCACCGAGUCGGUGCGUCUGUCAUUCAC | |
| | PAM | CGAUUUU | |
| RNACS5864 | TRAC4_ | GAGAAUCAAAAUCGGUGAAUGUUUUAGAGCUAGAAAUAGCAAGUUAAAAUAAGG | 17019 |
| | P9R10_del5- | CUAGUCCGUUAUCAACUUGAAAAAGUGGCACCGAGUCGGUGCGUCUGUCAUUCAC | |
| | PAM | CGAUUU | |
| RNACS5865 | TRAC4_ | GAGAAUCAAAAUCGGUGAAUGUUUUAGAGCUAGAAAUAGCAAGUUAAAAUAAGG | 17020 |
| | P8R10_del5- | CUAGUCCGUUAUCAACUUGAAAAAGUGGCACCGAGUCGGUGCGUCUGUCAUUCAC | |
| | PAM | CGAUU | |
| RNACS5866 | TRAC4_ | GAGAAUCAAAAUCGGUGAAUGUUUUAGAGCUAGAAAUAGCAAGUUAAAAUAAGG | 17021 |
| | P13R12_del5- | CUAGUCCGUUAUCAACUUGAAAAAGUGGCACCGAGUCGGUGCAAGUCUGUCAUUC | |
| | PAM | ACCGAUUUUGAU | |
| RNACS5867 | TRAC4_ | GAGAAUCAAAAUCGGUGAAUGUUUUAGAGCUAGAAAUAGCAAGUUAAAAUAAGG | 17022 |
| | P12R12_del5- | CUAGUCCGUUAUCAACUUGAAAAAGUGGCACCGAGUCGGUGCAAGUCUGUCAUUC | |
| | PAM | ACCGAUUUUGA | |
| RNACS5868 | TRAC4_ | GAGAAUCAAAAUCGGUGAAUGUUUUAGAGCUAGAAAUAGCAAGUUAAAAUAAGG | 17023 |
| | P11R12_del5- | CUAGUCCGUUAUCAACUUGAAAAAGUGGCACCGAGUCGGUGCAAGUCUGUCAUUC | |
| | PAM | ACCGAUUUUG | |
| RNACS5869 | TRAC4_ | GAGAAUCAAAAUCGGUGAAUGUUUUAGAGCUAGAAAUAGCAAGUUAAAAUAAGG | 17024 |
| | P10R12_del5- | CUAGUCCGUUAUCAACUUGAAAAAGUGGCACCGAGUCGGUGCAAGUCUGUCAUUC | |
| | PAM | ACCGAUUUU | |
| RNACS5870 | TRAC4_ | GAGAAUCAAAAUCGGUGAAUGUUUUAGAGCUAGAAAUAGCAAGUUAAAAUAAGG | 17025 |
| | P9R12 de15- | CUAGUCCGUUAUCAACUUGAAAAAGUGGCACCGAGUCGGUGCAAGUCUGUCAUUC | |
| | PAM | ACCGAUUU | |
| RNACS5871 | TRAC4_ | GAGAAUCAAAAUCGGUGAAUGUUUUAGAGCUAGAAAUAGCAAGUUAAAAUAAGG | 17026 |
| | P8R12_del5- | CUAGUCCGUUAUCAACUUGAAAAAGUGGCACCGAGUCGGUGCAAGUCUGUCAUUC | |
| | PAM | ACCGAUU | |
| RNACS5872 | TRAC4_ | GAGAAUCAAAAUCGGUGAAUGUUUUAGAGCUAGAAAUAGCAAGUUAAAAUAAGG | 17027 |
| | P13R14_del5- | CUAGUCCGUUAUCAACUUGAAAAAGUGGCACCGAGUCGGUGCACAAGUCUGUCAU | |
| | PAM | UCACCGAUUUUGAU | |
| RNACS5873 | TRAC4_ | GAGAAUCAAAAUCGGUGAAUGUUUUAGAGCUAGAAAUAGCAAGUUAAAAUAAGG | 17028 |
| | P12R14_del5- | CUAGUCCGUUAUCAACUUGAAAAAGUGGCACCGAGUCGGUGCACAAGUCUGUCAU | |
| | PAM | UCACCGAUUUUGA | |
| RNACS5874 | TRAC4_ | GAGAAUCAAAAUCGGUGAAUGUUUUAGAGCUAGAAAUAGCAAGUUAAAAUAAGG | 17029 |
| | P11R14_del5- | CUAGUCCGUUAUCAACUUGAAAAAGUGGCACCGAGUCGGUGCACAAGUCUGUCAU | |
| | PAM | UCACCGAUUUUG | |
| RNACS5875 | TRAC4_ | GAGAAUCAAAAUCGGUGAAUGUUUUAGAGCUAGAAAUAGCAAGUUAAAAUAAGG | 17030 |
| | P10R14_del5- | CUAGUCCGUUAUCAACUUGAAAAAGUGGCACCGAGUCGGUGCACAAGUCUGUCAU | |
| | PAM | UCACCGAUUUU | |
| RNACS5876 | TRAC4_ | GAGAAUCAAAAUCGGUGAAUGUUUUAGAGCUAGAAAUAGCAAGUUAAAAUAAGG | 17031 |
| | P9R14_del5- | CUAGUCCGUUAUCAACUUGAAAAAGUGGCACCGAGUCGGUGCACAAGUCUGUCAU | |
| | PAM | UCACCGAUUU | |
| RNACS5877 | TRAC4_ | GAGAAUCAAAAUCGGUGAAUGUUUUAGAGCUAGAAAUAGCAAGUUAAAAUAAGG | 17032 |
| | P8R14_del5- | CUAGUCCGUUAUCAACUUGAAAAAGUGGCACCGAGUCGGUGCACAAGUCUGUCAU | |
| | PAM | UCACCGAUU | |
| RNACS5878 | TRAC4_ | GAGAAUCAAAAUCGGUGAAUGUUUUAGAGCUAGAAAUAGCAAGUUAAAAUAAGG | 17033 |
| | P13R16_del5- | CUAGUCCGUUAUCAACUUGAAAAAGUGGCACCGAGUCGGUGCUGACAAGUCUGUC | |
| | PAM | AUUCACCGAUUUUGAU | |
| RNACS5879 | TRAC4_ | GAGAAUCAAAAUCGGUGAAUGUUUUAGAGCUAGAAAUAGCAAGUUAAAAUAAGG | 17034 |
| | P12R16_del5- | CUAGUCCGUUAUCAACUUGAAAAAGUGGCACCGAGUCGGUGCUGACAAGUCUGUC | |
| | PAM | AUUCACCGAUUUUGA | |
| RNACS5880 | TRAC4_ | GAGAAUCAAAAUCGGUGAAUGUUUUAGAGCUAGAAAUAGCAAGUUAAAAUAAGG | 17035 |
| | P11R16_del5- | CUAGUCCGUUAUCAACUUGAAAAAGUGGCACCGAGUCGGUGCUGACAAGUCUGUC | |
| | PAM | AUUCACCGAUUUUG | |
| RNACS5881 | TRAC4_ | GAGAAUCAAAAUCGGUGAAUGUUUUAGAGCUAGAAAUAGCAAGUUAAAAUAAGG | 17036 |
| | P10R16_del5- | CUAGUCCGUUAUCAACUUGAAAAAGUGGCACCGAGUCGGUGCUGACAAGUCUGUC | |
| | PAM | AUUCACCGAUUUU | |
| RNACS5882 | TRAC4_ | GAGAAUCAAAAUCGGUGAAUGUUUUAGAGCUAGAAAUAGCAAGUUAAAAUAAGG | 17037 |
| | P9R16_del5- | CUAGUCCGUUAUCAACUUGAAAAAGUGGCACCGAGUCGGUGCUGACAAGUCUGUC | |
| | PAM | AUUCACCGAUUU | |
| RNACS5883 | TRAC4_ | GAGAAUCAAAAUCGGUGAAUGUUUUAGAGCUAGAAAUAGCAAGUUAAAAUAAGG | 17038 |
| | P8R16_del5- | CUAGUCCGUUAUCAACUUGAAAAAGUGGCACCGAGUCGGUGCUGACAAGUCUGUC | |
| | PAM | AUUCACCGAUU | |
| RNACS5884 | TRAC4_ | GAGAAUCAAAAUCGGUGAAUGUUUUAGAGCUAGAAAUAGCAAGUUAAAAUAAGG | 17039 |
| | P13R18_del5- | CUAGUCCGUUAUCAACUUGAAAAAGUGGCACCGAGUCGGUGCAGUGACAAGUCUG | |
| | PAM | UCAUUCACCGAUUUUGAU | |
| RNACS5885 | TRAC4_ | GAGAAUCAAAAUCGGUGAAUGUUUUAGAGCUAGAAAUAGCAAGUUAAAAUAAGG | 17040 |
| | P12R18_del5- | CUAGUCCGUUAUCAACUUGAAAAAGUGGCACCGAGUCGGUGCAGUGACAAGUCUG | |
| | PAM | UCAUUCACCGAUUUUGA | |
| RNACS5886 | TRAC4_ | GAGAAUCAAAAUCGGUGAAUGUUUUAGAGCUAGAAAUAGCAAGUUAAAAUAAGG | 17041 |
| | P11R18_del5- | CUAGUCCGUUAUCAACUUGAAAAAGUGGCACCGAGUCGGUGCAGUGACAAGUCUG | |
| | PAM | UCAUUCACCGAUUUUG | |
| RNACS5887 | TRAC4_ | GAGAAUCAAAAUCGGUGAAUGUUUUAGAGCUAGAAAUAGCAAGUUAAAAUAAGG | 17042 |
| | P10R18_del5- | CUAGUCCGUUAUCAACUUGAAAAAGUGGCACCGAGUCGGUGCAGUGACAAGUCUG | |
| | PAM | UCAUUCACCGAUUUU | |
| RNACS5888 | TRAC4_ | GAGAAUCAAAAUCGGUGAAUGUUUUAGAGCUAGAAAUAGCAAGUUAAAAUAAGG | 17043 |
| | P9R18_del5- | CUAGUCCGUUAUCAACUUGAAAAAGUGGCACCGAGUCGGUGCAGUGACAAGUCUG | |
| | PAM | UCAUUCACCGAUUU | |
| RNACS5889 | TRAC4_ | GAGAAUCAAAAUCGGUGAAUGUUUUAGAGCUAGAAAUAGCAAGUUAAAAUAAGG | 17044 |
| | P8R18_del5- | CUAGUCCGUUAUCAACUUGAAAAAGUGGCACCGAGUCGGUGCAGUGACAAGUCUG | |
| | PAM | UCAUUCACCGAUU | |
| RNACS5890 | TRAC4_ | GAGAAUCAAAAUCGGUGAAUGUUUUAGAGCUAGAAAUAGCAAGUUAAAAUAAGG | 17045 |
| | P13R20_del5- | CUAGUCCGUUAUCAACUUGAAAAAGUGGCACCGAGUCGGUGCCCAGUGACAAGUC | |
| | PAM | UGUCAUUCACCGAUUUUGAU | |
| RNACS5891 | TRAC4_ | GAGAAUCAAAAUCGGUGAAUGUUUUAGAGCUAGAAAUAGCAAGUUAAAAUAAGG | 17046 |
| | P12R20_del5- | CUAGUCCGUUAUCAACUUGAAAAAGUGGCACCGAGUCGGUGCCCAGUGACAAGUC | |
| | PAM | UGUCAUUCACCGAUUUUGA | |
| RNACS5892 | TRAC4_ | GAGAAUCAAAAUCGGUGAAUGUUUUAGAGCUAGAAAUAGCAAGUUAAAAUAAGG | 17047 |
| | P11R20_del5- | CUAGUCCGUUAUCAACUUGAAAAAGUGGCACCGAGUCGGUGCCCAGUGACAAGUC | |
| | PAM | UGUCAUUCACCGAUUUUG | |
| RNACS5893 | TRAC4_ | GAGAAUCAAAAUCGGUGAAUGUUUUAGAGCUAGAAAUAGCAAGUUAAAAUAAGG | 17048 |
| | P10R20_del5- | CUAGUCCGUUAUCAACUUGAAAAAGUGGCACCGAGUCGGUGCCCAGUGACAAGUC | |
| | PAM | UGUCAUUCACCGAUUUU | |
| RNACS5894 | TRAC4_ | GAGAAUCAAAAUCGGUGAAUGUUUUAGAGCUAGAAAUAGCAAGUUAAAAUAAGG | 17049 |
| | P9R20_del5- | CUAGUCCGUUAUCAACUUGAAAAAGUGGCACCGAGUCGGUGCCCAGUGACAAGUC | |
| | PAM | UGUCAUUCACCGAUUU | |
| RNACS5895 | TRAC4_ | GAGAAUCAAAAUCGGUGAAUGUUUUAGAGCUAGAAAUAGCAAGUUAAAAUAAGG | 17050 |
| | P8R20_del5- | CUAGUCCGUUAUCAACUUGAAAAAGUGGCACCGAGUCGGUGCCCAGUGACAAGUC | |
| | PAM | UGUCAUUCACCGAUU | |
| RNACS5896 | TRAC4_ | GAGAAUCAAAAUCGGUGAAUGUUUUAGAGCUAGAAAUAGCAAGUUAAAAUAAGG | 17051 |
| | P13R8_del7- | CUAGUCCGUUAUCAACUUGAAAAAGUGGCACCGAGUCGGUGCGUCUGAUUCACCG | |
| | PAM | AUUUUGAU | |
| RNACS5897 | TRAC4_ | GAGAAUCAAAAUCGGUGAAUGUUUUAGAGCUAGAAAUAGCAAGUUAAAAUAAGG | 17052 |
| | P12R8_del7- | CUAGUCCGUUAUCAACUUGAAAAAGUGGCACCGAGUCGGUGCGUCUGAUUCACCG | |
| | PAM | AUUUUGA | |
| RNACS5898 | TRAC4_ | GAGAAUCAAAAUCGGUGAAUGUUUUAGAGCUAGAAAUAGCAAGUUAAAAUAAGG | 17053 |
| | P11R8_del7- | CUAGUCCGUUAUCAACUUGAAAAAGUGGCACCGAGUCGGUGCGUCUGAUUCACCG | |
| | PAM | AUUUUG | |
| RNACS5899 | TRAC4_ | GAGAAUCAAAAUCGGUGAAUGUUUUAGAGCUAGAAAUAGCAAGUUAAAAUAAGG | 17054 |
| | P10R8_del7- | CUAGUCCGUUAUCAACUUGAAAAAGUGGCACCGAGUCGGUGCGUCUGAUUCACCG | |
| | PAM | AUUUU | |
| RNACS5900 | TRAC4_ | GAGAAUCAAAAUCGGUGAAUGUUUUAGAGCUAGAAAUAGCAAGUUAAAAUAAGG | 17055 |
| | P9R8_del7- | CUAGUCCGUUAUCAACUUGAAAAAGUGGCACCGAGUCGGUGCGUCUGAUUCACCG | |
| | PAM | AUUU | |
| RNACS5901 | TRAC4_ | GAGAAUCAAAAUCGGUGAAUGUUUUAGAGCUAGAAAUAGCAAGUUAAAAUAAGG | 17056 |
| | P8R8_del7- | CUAGUCCGUUAUCAACUUGAAAAAGUGGCACCGAGUCGGUGCGUCUGAUUCACCG | |
| | PAM | AUU | |
| RNACS5902 | TRAC4_ | GAGAAUCAAAAUCGGUGAAUGUUUUAGAGCUAGAAAUAGCAAGUUAAAAUAAGG | 17057 |
| | P13R10_del7- | CUAGUCCGUUAUCAACUUGAAAAAGUGGCACCGAGUCGGUGCAAGUCUGAUUCAC | |
| | PAM | CGAUUUUGAU | |
| RNACS5903 | TRAC4_ | GAGAAUCAAAAUCGGUGAAUGUUUUAGAGCUAGAAAUAGCAAGUUAAAAUAAGG | 17058 |
| | P12R10_del7- | CUAGUCCGUUAUCAACUUGAAAAAGUGGCACCGAGUCGGUGCAAGUCUGAUUCAC | |
| | PAM | CGAUUUUGA | |
| RNACS5904 | TRAC4_ | GAGAAUCAAAAUCGGUGAAUGUUUUAGAGCUAGAAAUAGCAAGUUAAAAUAAGG | 17059 |
| | P11R10_del7- | CUAGUCCGUUAUCAACUUGAAAAAGUGGCACCGAGUCGGUGCAAGUCUGAUUCAC | |
| | PAM | CGAUUUUG | |
| RNACS5905 | TRAC4_ | GAGAAUCAAAAUCGGUGAAUGUUUUAGAGCUAGAAAUAGCAAGUUAAAAUAAGG | 17060 |
| | P10R10_del7- | CUAGUCCGUUAUCAACUUGAAAAAGUGGCACCGAGUCGGUGCAAGUCUGAUUCAC | |
| | PAM | CGAUUUU | |
| RNACS5906 | TRAC4_ | GAGAAUCAAAAUCGGUGAAUGUUUUAGAGCUAGAAAUAGCAAGUUAAAAUAAGG | 17061 |
| | P9R10_del7- | CUAGUCCGUUAUCAACUUGAAAAAGUGGCACCGAGUCGGUGCAAGUCUGAUUCAC | |
| | PAM | CGAUUU | |
| RNACS5907 | TRAC4_ | GAGAAUCAAAAUCGGUGAAUGUUUUAGAGCUAGAAAUAGCAAGUUAAAAUAAGG | 17062 |
| | P8R10_del7- | CUAGUCCGUUAUCAACUUGAAAAAGUGGCACCGAGUCGGUGCAAGUCUGAUUCAC | |
| | PAM | CGAUU | |
| RNACS5908 | TRAC4_ | GAGAAUCAAAAUCGGUGAAUGUUUUAGAGCUAGAAAUAGCAAGUUAAAAUAAGG | 17063 |
| | P13R12_del7- | CUAGUCCGUUAUCAACUUGAAAAAGUGGCACCGAGUCGGUGCACAAGUCUGAUUC | |
| | PAM | ACCGAUUUUGAU | |
| RNACS5909 | TRAC4_ | GAGAAUCAAAAUCGGUGAAUGUUUUAGAGCUAGAAAUAGCAAGUUAAAAUAAGG | 17064 |
| | P12R12_del7- | CUAGUCCGUUAUCAACUUGAAAAAGUGGCACCGAGUCGGUGCACAAGUCUGAUUC | |
| | PAM | ACCGAUUUUGA | |
| RNACS5910 | TRAC4_ | GAGAAUCAAAAUCGGUGAAUGUUUUAGAGCUAGAAAUAGCAAGUUAAAAUAAGG | 17065 |
| | P11R12_del7- | CUAGUCCGUUAUCAACUUGAAAAAGUGGCACCGAGUCGGUGCACAAGUCUGAUUC | |
| | PAM | ACCGAUUUUG | |
| RNACS5911 | TRAC4_ | GAGAAUCAAAAUCGGUGAAUGUUUUAGAGCUAGAAAUAGCAAGUUAAAAUAAGG | 17066 |
| | P10R12_del7- | CUAGUCCGUUAUCAACUUGAAAAAGUGGCACCGAGUCGGUGCACAAGUCUGAUUC | |
| | PAM | ACCGAUUUU | |
| RNACS5912 | TRAC4_ | GAGAAUCAAAAUCGGUGAAUGUUUUAGAGCUAGAAAUAGCAAGUUAAAAUAAGG | 17067 |
| | P9R12_del7- | CUAGUCCGUUAUCAACUUGAAAAAGUGGCACCGAGUCGGUGCACAAGUCUGAUUC | |
| | PAM | ACCGAUUU | |
| RNACS5913 | TRAC4_ | GAGAAUCAAAAUCGGUGAAUGUUUUAGAGCUAGAAAUAGCAAGUUAAAAUAAGG | 17068 |
| | P8R12_del7- | CUAGUCCGUUAUCAACUUGAAAAAGUGGCACCGAGUCGGUGCACAAGUCUGAUUC | |
| | PAM | ACCGAUU | |
| RNACS5914 | TRAC4_ | GAGAAUCAAAAUCGGUGAAUGUUUUAGAGCUAGAAAUAGCAAGUUAAAAUAAGG | 17069 |
| | P13R14_del7- | CUAGUCCGUUAUCAACUUGAAAAAGUGGCACCGAGUCGGUGCUGACAAGUCUGAU | |
| | PAM | UCACCGAUUUUGAU | |
| RNACS5915 | TRAC4_ | GAGAAUCAAAAUCGGUGAAUGUUUUAGAGCUAGAAAUAGCAAGUUAAAAUAAGG | 17070 |
| | P12R14_del7- | CUAGUCCGUUAUCAACUUGAAAAAGUGGCACCGAGUCGGUGCUGACAAGUCUGAU | |
| | PAM | UCACCGAUUUUGA | |
| RNACS5916 | TRAC4_ | GAGAAUCAAAAUCGGUGAAUGUUUUAGAGCUAGAAAUAGCAAGUUAAAAUAAGG | 17071 |
| | P11R14_del7- | CUAGUCCGUUAUCAACUUGAAAAAGUGGCACCGAGUCGGUGCUGACAAGUCUGAU | |
| | PAM | UCACCGAUUUUG | |
| RNACS5917 | TRAC4_ | GAGAAUCAAAAUCGGUGAAUGUUUUAGAGCUAGAAAUAGCAAGUUAAAAUAAGG | 17072 |
| | P10R14_del7- | CUAGUCCGUUAUCAACUUGAAAAAGUGGCACCGAGUCGGUGCUGACAAGUCUGAU | |
| | PAM | UCACCGAUUUU | |
| RNACS5918 | TRAC4_ | GAGAAUCAAAAUCGGUGAAUGUUUUAGAGCUAGAAAUAGCAAGUUAAAAUAAGG | 17073 |
| | P9R14_del7- | CUAGUCCGUUAUCAACUUGAAAAAGUGGCACCGAGUCGGUGCUGACAAGUCUGAU | |
| | PAM | UCACCGAUUU | |
| RNACS5919 | TRAC4_ | GAGAAUCAAAAUCGGUGAAUGUUUUAGAGCUAGAAAUAGCAAGUUAAAAUAAGG | 17074 |
| | P8R14_del7- | CUAGUCCGUUAUCAACUUGAAAAAGUGGCACCGAGUCGGUGCUGACAAGUCUGAU | |
| | PAM | UCACCGAUU | |
| RNACS5920 | TRAC4_ | GAGAAUCAAAAUCGGUGAAUGUUUUAGAGCUAGAAAUAGCAAGUUAAAAUAAGG | 17075 |
| | P13R16_del7- | CUAGUCCGUUAUCAACUUGAAAAAGUGGCACCGAGUCGGUGCAGUGACAAGUCUG | |
| | PAM | AUUCACCGAUUUUGAU | |
| RNACS5921 | TRAC4_ | GAGAAUCAAAAUCGGUGAAUGUUUUAGAGCUAGAAAUAGCAAGUUAAAAUAAGG | 17076 |
| | P12R16_del7- | CUAGUCCGUUAUCAACUUGAAAAAGUGGCACCGAGUCGGUGCAGUGACAAGUCUG | |
| | PAM | AUUCACCGAUUUUGA | |
| RNACS5922 | TRAC4_ | GAGAAUCAAAAUCGGUGAAUGUUUUAGAGCUAGAAAUAGCAAGUUAAAAUAAGG | 17077 |
| | P11R16_del7- | CUAGUCCGUUAUCAACUUGAAAAAGUGGCACCGAGUCGGUGCAGUGACAAGUCUG | |
| | PAM | AUUCACCGAUUUUG | |
| RNACS5923 | TRAC4_ | GAGAAUCAAAAUCGGUGAAUGUUUUAGAGCUAGAAAUAGCAAGUUAAAAUAAGG | 17078 |
| | P10R16_del7- | CUAGUCCGUUAUCAACUUGAAAAAGUGGCACCGAGUCGGUGCAGUGACAAGUCUG | |
| | PAM | AUUCACCGAUUUU | |
| RNACS5924 | TRAC4_ | GAGAAUCAAAAUCGGUGAAUGUUUUAGAGCUAGAAAUAGCAAGUUAAAAUAAGG | 17079 |
| | P9R16_del7- | CUAGUCCGUUAUCAACUUGAAAAAGUGGCACCGAGUCGGUGCAGUGACAAGUCUG | |
| | PAM | AUUCACCGAUUU | |
| RNACS5925 | TRAC4_ | GAGAAUCAAAAUCGGUGAAUGUUUUAGAGCUAGAAAUAGCAAGUUAAAAUAAGG | 17080 |
| | P8R16_del7- | CUAGUCCGUUAUCAACUUGAAAAAGUGGCACCGAGUCGGUGCAGUGACAAGUCUG | |
| | PAM | AUUCACCGAUU | |
| RNACS5926 | TRAC4_ | GAGAAUCAAAAUCGGUGAAUGUUUUAGAGCUAGAAAUAGCAAGUUAAAAUAAGG | 17081 |
| | P13R18_del7- | CUAGUCCGUUAUCAACUUGAAAAAGUGGCACCGAGUCGGUGCCCAGUGACAAGUC | |
| | PAM | UGAUUCACCGAUUUUGAU | |
| RNACS5927 | TRAC4_ | GAGAAUCAAAAUCGGUGAAUGUUUUAGAGCUAGAAAUAGCAAGUUAAAAUAAGG | 17082 |
| | P12R18_del7- | CUAGUCCGUUAUCAACUUGAAAAAGUGGCACCGAGUCGGUGCCCAGUGACAAGUC | |
| | PAM | UGAUUCACCGAUUUUGA | |
| RNACS5928 | TRAC4_ | GAGAAUCAAAAUCGGUGAAUGUUUUAGAGCUAGAAAUAGCAAGUUAAAAUAAGG | 17083 |
| | P11R18_del7- | CUAGUCCGUUAUCAACUUGAAAAAGUGGCACCGAGUCGGUGCCCAGUGACAAGUC | |
| | PAM | UGAUUCACCGAUUUUG | |
| RNACS5929 | TRAC4_ | GAGAAUCAAAAUCGGUGAAUGUUUUAGAGCUAGAAAUAGCAAGUUAAAAUAAGG | 17084 |
| | P10R18_del7- | CUAGUCCGUUAUCAACUUGAAAAAGUGGCACCGAGUCGGUGCCCAGUGACAAGUC | |
| | PAM | UGAUUCACCGAUUUU | |
| RNACS5930 | TRAC4_ | GAGAAUCAAAAUCGGUGAAUGUUUUAGAGCUAGAAAUAGCAAGUUAAAAUAAGG | 17085 |
| | P9R18_del7- | CUAGUCCGUUAUCAACUUGAAAAAGUGGCACCGAGUCGGUGCCCAGUGACAAGUC | |
| | PAM | UGAUUCACCGAUUU | |
| RNACS5931 | TRAC4_ | GAGAAUCAAAAUCGGUGAAUGUUUUAGAGCUAGAAAUAGCAAGUUAAAAUAAGG | 17086 |
| | P8R18_del7- | CUAGUCCGUUAUCAACUUGAAAAAGUGGCACCGAGUCGGUGCCCAGUGACAAGUC | |
| | PAM | UGAUUCACCGAUU | |
| RNACS5932 | TRAC4_ | GAGAAUCAAAAUCGGUGAAUGUUUUAGAGCUAGAAAUAGCAAGUUAAAAUAAGG | 17087 |
| | P13R20_del7- | CUAGUCCGUUAUCAACUUGAAAAAGUGGCACCGAGUCGGUGCAUCCAGUGACAAG | |
| | PAM | UCUGAUUCACCGAUUUUGAU | |
| RNACS5933 | TRAC4_ | GAGAAUCAAAAUCGGUGAAUGUUUUAGAGCUAGAAAUAGCAAGUUAAAAUAAGG | 17088 |
| | P12R20_del7- | CUAGUCCGUUAUCAACUUGAAAAAGUGGCACCGAGUCGGUGCAUCCAGUGACAAG | |
| | PAM | UCUGAUUCACCGAUUUUGA | |
| RNACS5934 | TRAC4_ | GAGAAUCAAAAUCGGUGAAUGUUUUAGAGCUAGAAAUAGCAAGUUAAAAUAAGG | 17089 |
| | P11R20_del7- | CUAGUCCGUUAUCAACUUGAAAAAGUGGCACCGAGUCGGUGCAUCCAGUGACAAG | |
| | PAM | UCUGAUUCACCGAUUUUG | |
| RNACS5935 | TRAC4_ | GAGAAUCAAAAUCGGUGAAUGUUUUAGAGCUAGAAAUAGCAAGUUAAAAUAAGG | 17090 |
| | P10R20_del7- | CUAGUCCGUUAUCAACUUGAAAAAGUGGCACCGAGUCGGUGCAUCCAGUGACAAG | |
| | PAM | UCUGAUUCACCGAUUUU | |
| RNACS5936 | TRAC4_ | GAGAAUCAAAAUCGGUGAAUGUUUUAGAGCUAGAAAUAGCAAGUUAAAAUAAGG | 17091 |
| | P9R20_del7- | CUAGUCCGUUAUCAACUUGAAAAAGUGGCACCGAGUCGGUGCAUCCAGUGACAAG | |
| | PAM | UCUGAUUCACCGAUUU | |
| RNACS5937 | TRAC4_ | GAGAAUCAAAAUCGGUGAAUGUUUUAGAGCUAGAAAUAGCAAGUUAAAAUAAGG | 17092 |
| | P8R20_del7- | CUAGUCCGUUAUCAACUUGAAAAAGUGGCACCGAGUCGGUGCAUCCAGUGACAAG | |
| | PAM | UCUGAUUCACCGAUU | |
| RNACS5938 | TRAC4_ | GAGAAUCAAAAUCGGUGAAUGUUUUAGAGCUAGAAAUAGCAAGUUAAAAUAAGG | 17093 |
| | P13R10_ins5- | CUAGUCCGUUAUCAACUUGAAAAAGUGGCACCGAGUCGGUGCCUAUUUGAUGCAC | |
| | nick-v1 | CGAUUUUGAU | |
| RNACS5939 | TRAC4_ | GAGAAUCAAAAUCGGUGAAUGUUUUAGAGCUAGAAAUAGCAAGUUAAAAUAAGG | 17094 |
| | P12R10_ins5- | CUAGUCCGUUAUCAACUUGAAAAAGUGGCACCGAGUCGGUGCCUAUUUGAUGCAC | |
| | nick-v1 | CGAUUUUGA | |
| RNACS5940 | TRAC4_ | GAGAAUCAAAAUCGGUGAAUGUUUUAGAGCUAGAAAUAGCAAGUUAAAAUAAGG | 17095 |
| | P11R10_ins5- | CUAGUCCGUUAUCAACUUGAAAAAGUGGCACCGAGUCGGUGCCUAUUUGAUGCAC | |
| | nick-v1 | CGAUUUUG | |
| RNACS5941 | TRAC4_ | GAGAAUCAAAAUCGGUGAAUGUUUUAGAGCUAGAAAUAGCAAGUUAAAAUAAGG | 17096 |
| | P10R10_ins5- | CUAGUCCGUUAUCAACUUGAAAAAGUGGCACCGAGUCGGUGCCUAUUUGAUGCAC | |
| | nick-v1 | CGAUUUU | |
| RNACS5942 | TRAC4_ | GAGAAUCAAAAUCGGUGAAUGUUUUAGAGCUAGAAAUAGCAAGUUAAAAUAAGG | 17097 |
| | P9R10_ins5- | CUAGUCCGUUAUCAACUUGAAAAAGUGGCACCGAGUCGGUGCCUAUUUGAUGCAC | |
| | nick-v1 | CGAUUU | |
| RNACS5943 | TRAC4_ | GAGAAUCAAAAUCGGUGAAUGUUUUAGAGCUAGAAAUAGCAAGUUAAAAUAAGG | 17098 |
| | P8R10_ins5- | CUAGUCCGUUAUCAACUUGAAAAAGUGGCACCGAGUCGGUGCCUAUUUGAUGCAC | |
| | nick-v1 | CGAUU | |
| RNACS5944 | TRAC4_ | GAGAAUCAAAAUCGGUGAAUGUUUUAGAGCUAGAAAUAGCAAGUUAAAAUAAGG | 17099 |
| | P13R12_ins5- | CUAGUCCGUUAUCAACUUGAAAAAGUGGCACCGAGUCGGUGCGCCUAUUUGAUGC | |
| | nick-v1 | ACCGAUUUUGAU | |
| RNACS5945 | TRAC4_ | GAGAAUCAAAAUCGGUGAAUGUUUUAGAGCUAGAAAUAGCAAGUUAAAAUAAGG | 17100 |
| | P12R12_ins5- | CUAGUCCGUUAUCAACUUGAAAAAGUGGCACCGAGUCGGUGCGCCUAUUUGAUGC | |
| | nick-v1 | ACCGAUUUUGA | |
| RNACS5946 | TRAC4_ | GAGAAUCAAAAUCGGUGAAUGUUUUAGAGCUAGAAAUAGCAAGUUAAAAUAAGG | 17101 |
| | P11R12_ins5- | CUAGUCCGUUAUCAACUUGAAAAAGUGGCACCGAGUCGGUGCGCCUAUUUGAUGC | |
| | nick-v1 | ACCGAUUUUG | |
| RNACS5947 | TRAC4_ | GAGAAUCAAAAUCGGUGAAUGUUUUAGAGCUAGAAAUAGCAAGUUAAAAUAAGG | 17102 |
| | P10R12_ins5- | CUAGUCCGUUAUCAACUUGAAAAAGUGGCACCGAGUCGGUGCGCCUAUUUGAUGC | |
| | nick-v1 | ACCGAUUUU | |
| RNACS5948 | TRAC4_ | GAGAAUCAAAAUCGGUGAAUGUUUUAGAGCUAGAAAUAGCAAGUUAAAAUAAGG | 17103 |
| | P9R12_ins5- | CUAGUCCGUUAUCAACUUGAAAAAGUGGCACCGAGUCGGUGCGCCUAUUUGAUGC | |
| | nick-v1 | ACCGAUUU | |
| RNACS5949 | TRAC4_ | GAGAAUCAAAAUCGGUGAAUGUUUUAGAGCUAGAAAUAGCAAGUUAAAAUAAGG | 17104 |
| | P8R12_ins5- | CUAGUCCGUUAUCAACUUGAAAAAGUGGCACCGAGUCGGUGCGCCUAUUUGAUGC | |
| | nick-v1 | ACCGAUU | |
| RNACS5950 | TRAC4_ | GAGAAUCAAAAUCGGUGAAUGUUUUAGAGCUAGAAAUAGCAAGUUAAAAUAAGG | 17105 |
| | P13R14_ins5- | CUAGUCCGUUAUCAACUUGAAAAAGUGGCACCGAGUCGGUGCCUGCCUAUUUGAU | |
| | nick-v1 | GCACCGAUUUUGAU | |
| RNACS5951 | TRAC4_ | GAGAAUCAAAAUCGGUGAAUGUUUUAGAGCUAGAAAUAGCAAGUUAAAAUAAGG | 17106 |
| | P12R14_ins5- | CUAGUCCGUUAUCAACUUGAAAAAGUGGCACCGAGUCGGUGCCUGCCUAUUUGAU | |
| | nick-v1 | GCACCGAUUUUGA | |
| RNACS5952 | TRAC4_ | GAGAAUCAAAAUCGGUGAAUGUUUUAGAGCUAGAAAUAGCAAGUUAAAAUAAGG | 17107 |
| | P11R14_ins5- | CUAGUCCGUUAUCAACUUGAAAAAGUGGCACCGAGUCGGUGCCUGCCUAUUUGAU | |
| | nick-v1 | GCACCGAUUUUG | |
| RNACS5953 | TRAC4_ | GAGAAUCAAAAUCGGUGAAUGUUUUAGAGCUAGAAAUAGCAAGUUAAAAUAAGG | 17108 |
| | P10R14_ins5- | CUAGUCCGUUAUCAACUUGAAAAAGUGGCACCGAGUCGGUGCCUGCCUAUUUGAU | |
| | nick-v1 | GCACCGAUUUU | |
| RNACS5954 | TRAC4_ | GAGAAUCAAAAUCGGUGAAUGUUUUAGAGCUAGAAAUAGCAAGUUAAAAUAAGG | 17109 |
| | P9R14_ins5- | CUAGUCCGUUAUCAACUUGAAAAAGUGGCACCGAGUCGGUGCCUGCCUAUUUGAU | |
| | nick-v1 | GCACCGAUUU | |
| RNACS5955 | TRAC4_ | GAGAAUCAAAAUCGGUGAAUGUUUUAGAGCUAGAAAUAGCAAGUUAAAAUAAGG | 17110 |
| | P8R14_ins5- | CUAGUCCGUUAUCAACUUGAAAAAGUGGCACCGAGUCGGUGCCUGCCUAUUUGAU | |
| | nick-v1 | GCACCGAUU | |
| RNACS5956 | TRAC4_ | GAGAAUCAAAAUCGGUGAAUGUUUUAGAGCUAGAAAUAGCAAGUUAAAAUAAGG | 17111 |
| | P13R16_ins5- | CUAGUCCGUUAUCAACUUGAAAAAGUGGCACCGAGUCGGUGCGUCUGCCUAUUUG | |
| | nick-v1 | AUGCACCGAUUUUGAU | |
| RNACS5957 | TRAC4_ | GAGAAUCAAAAUCGGUGAAUGUUUUAGAGCUAGAAAUAGCAAGUUAAAAUAAGG | 17112 |
| | P12R16_ins5- | CUAGUCCGUUAUCAACUUGAAAAAGUGGCACCGAGUCGGUGCGUCUGCCUAUUUG | |
| | nick-v1 | AUGCACCGAUUUUGA | |
| RNACS5958 | TRAC4_ | GAGAAUCAAAAUCGGUGAAUGUUUUAGAGCUAGAAAUAGCAAGUUAAAAUAAGG | 17113 |
| | P11R16_ins5- | CUAGUCCGUUAUCAACUUGAAAAAGUGGCACCGAGUCGGUGCGUCUGCCUAUUUG | |
| | nick-v1 | AUGCACCGAUUUUG | |
| RNACS5959 | TRAC4_ | GAGAAUCAAAAUCGGUGAAUGUUUUAGAGCUAGAAAUAGCAAGUUAAAAUAAGG | 17114 |
| | P10R16_ins5- | CUAGUCCGUUAUCAACUUGAAAAAGUGGCACCGAGUCGGUGCGUCUGCCUAUUUG | |
| | nick-v1 | AUGCACCGAUUUU | |
| RNACS5960 | TRAC4_ | GAGAAUCAAAAUCGGUGAAUGUUUUAGAGCUAGAAAUAGCAAGUUAAAAUAAGG | 17115 |
| | P9R16_ins5- | CUAGUCCGUUAUCAACUUGAAAAAGUGGCACCGAGUCGGUGCGUCUGCCUAUUUG | |
| | nick-v1 | AUGCACCGAUUU | |
| RNACS5961 | TRAC4_ | GAGAAUCAAAAUCGGUGAAUGUUUUAGAGCUAGAAAUAGCAAGUUAAAAUAAGG | 17116 |
| | P8R16_ins5- | CUAGUCCGUUAUCAACUUGAAAAAGUGGCACCGAGUCGGUGCGUCUGCCUAUUUG | |
| | nick-v1 | AUGCACCGAUU | |
| RNACS5962 | TRAC4_ | GAGAAUCAAAAUCGGUGAAUGUUUUAGAGCUAGAAAUAGCAAGUUAAAAUAAGG | 17117 |
| | P13R18_ins5- | CUAGUCCGUUAUCAACUUGAAAAAGUGGCACCGAGUCGGUGCCUGUCUGCCUAUU | |
| | nick-v1 | UGAUGCACCGAUUUUGAU | |
| RNACS5963 | TRAC4_ | GAGAAUCAAAAUCGGUGAAUGUUUUAGAGCUAGAAAUAGCAAGUUAAAAUAAGG | 17118 |
| | P12R18_ins5- | CUAGUCCGUUAUCAACUUGAAAAAGUGGCACCGAGUCGGUGCCUGUCUGCCUAUU | |
| | nick-v1 | UGAUGCACCGAUUUUGA | |
| RNACS5964 | TRAC4_ | GAGAAUCAAAAUCGGUGAAUGUUUUAGAGCUAGAAAUAGCAAGUUAAAAUAAGG | 17119 |
| | P11R18_ins5- | CUAGUCCGUUAUCAACUUGAAAAAGUGGCACCGAGUCGGUGCCUGUCUGCCUAUU | |
| | nick-v1 | UGAUGCACCGAUUUUG | |
| RNACS5965 | TRAC4_ | GAGAAUCAAAAUCGGUGAAUGUUUUAGAGCUAGAAAUAGCAAGUUAAAAUAAGG | 17120 |
| | P10R18_ins5- | CUAGUCCGUUAUCAACUUGAAAAAGUGGCACCGAGUCGGUGCCUGUCUGCCUAUU | |
| | nick-v1 | UGAUGCACCGAUUUU | |
| RNACS5966 | TRAC4_ | GAGAAUCAAAAUCGGUGAAUGUUUUAGAGCUAGAAAUAGCAAGUUAAAAUAAGG | 17121 |
| | P9R18_ins5- | CUAGUCCGUUAUCAACUUGAAAAAGUGGCACCGAGUCGGUGCCUGUCUGCCUAUU | |
| | nick-v1 | UGAUGCACCGAUUU | |
| RNACS5967 | TRAC4_ | GAGAAUCAAAAUCGGUGAAUGUUUUAGAGCUAGAAAUAGCAAGUUAAAAUAAGG | 17122 |
| | P8R18_ins5- | CUAGUCCGUUAUCAACUUGAAAAAGUGGCACCGAGUCGGUGCCUGUCUGCCUAUU | |
| | nick-v1 | UGAUGCACCGAUU | |
| RNACS5968 | TRAC4_ | GAGAAUCAAAAUCGGUGAAUGUUUUAGAGCUAGAAAUAGCAAGUUAAAAUAAGG | 17123 |
| | P13R20_ins5- | CUAGUCCGUUAUCAACUUGAAAAAGUGGCACCGAGUCGGUGCGUCUGUCUGCCUA | |
| | nick-v1 | UUUGAUGCACCGAUUUUGAU | |
| RNACS5969 | TRAC4_ | GAGAAUCAAAAUCGGUGAAUGUUUUAGAGCUAGAAAUAGCAAGUUAAAAUAAGG | 17124 |
| | P12R20_ins5- | CUAGUCCGUUAUCAACUUGAAAAAGUGGCACCGAGUCGGUGCGUCUGUCUGCCUA | |
| | nick-v1 | UUUGAUGCACCGAUUUUGA | |
| RNACS5970 | TRAC4_ | GAGAAUCAAAAUCGGUGAAUGUUUUAGAGCUAGAAAUAGCAAGUUAAAAUAAGG | 17125 |
| | P11R20_ins5- | CUAGUCCGUUAUCAACUUGAAAAAGUGGCACCGAGUCGGUGCGUCUGUCUGCCUA | |
| | nick-v1 | UUUGAUGCACCGAUUUUG | |
| RNACS5971 | TRAC4_ | GAGAAUCAAAAUCGGUGAAUGUUUUAGAGCUAGAAAUAGCAAGUUAAAAUAAGG | 17126 |
| | P10R20_ins5- | CUAGUCCGUUAUCAACUUGAAAAAGUGGCACCGAGUCGGUGCGUCUGUCUGCCUA | |
| | nick-v1 | UUUGAUGCACCGAUUUU | |
| RNACS5972 | TRAC4_ | GAGAAUCAAAAUCGGUGAAUGUUUUAGAGCUAGAAAUAGCAAGUUAAAAUAAGG | 17127 |
| | P9R20_ins5- | CUAGUCCGUUAUCAACUUGAAAAAGUGGCACCGAGUCGGUGCGUCUGUCUGCCUA | |
| | nick-v1 | UUUGAUGCACCGAUUU | |
| RNACS5973 | TRAC4_ | GAGAAUCAAAAUCGGUGAAUGUUUUAGAGCUAGAAAUAGCAAGUUAAAAUAAGG | 17128 |
| | P8R20_ins5- | CUAGUCCGUUAUCAACUUGAAAAAGUGGCACCGAGUCGGUGCGUCUGUCUGCCUA | |
| | nick-v1 | UUUGAUGCACCGAUU | |
| RNACS5974 | TRAC4_ | GAGAAUCAAAAUCGGUGAAUGUUUUAGAGCUAGAAAUAGCAAGUUAAAAUAAGG | 17129 |
| | P13R22_ins5- | CUAGUCCGUUAUCAACUUGAAAAAGUGGCACCGAGUCGGUGCAAGUCUGUCUGCC | |
| | nick-v1 | UAUUUGAUGCACCGAUUUUGAU | |
| RNACS5975 | TRAC4_ | GAGAAUCAAAAUCGGUGAAUGUUUUAGAGCUAGAAAUAGCAAGUUAAAAUAAGG | 17130 |
| | P12R22_ins5- | CUAGUCCGUUAUCAACUUGAAAAAGUGGCACCGAGUCGGUGCAAGUCUGUCUGCC | |
| | nick-v1 | UAUUUGAUGCACCGAUUUUGA | |
| RNACS5976 | TRAC4_ | GAGAAUCAAAAUCGGUGAAUGUUUUAGAGCUAGAAAUAGCAAGUUAAAAUAAGG | 17131 |
| | P11R22_ins5- | CUAGUCCGUUAUCAACUUGAAAAAGUGGCACCGAGUCGGUGCAAGUCUGUCUGCC | |
| | nick-v1 | UAUUUGAUGCACCGAUUUUG | |
| RNACS5977 | TRAC4_ | GAGAAUCAAAAUCGGUGAAUGUUUUAGAGCUAGAAAUAGCAAGUUAAAAUAAGG | 17132 |
| | P10R22_ins5- | CUAGUCCGUUAUCAACUUGAAAAAGUGGCACCGAGUCGGUGCAAGUCUGUCUGCC | |
| | nick-v1 | UAUUUGAUGCACCGAUUUU | |
| RNACS5978 | TRAC4_ | GAGAAUCAAAAUCGGUGAAUGUUUUAGAGCUAGAAAUAGCAAGUUAAAAUAAGG | 17133 |
| | P9R22_ins5- | CUAGUCCGUUAUCAACUUGAAAAAGUGGCACCGAGUCGGUGCAAGUCUGUCUGCC | |
| | nick-v1 | UAUUUGAUGCACCGAUUU | |
| RNACS5979 | TRAC4_ | GAGAAUCAAAAUCGGUGAAUGUUUUAGAGCUAGAAAUAGCAAGUUAAAAUAAGG | 17134 |
| | P8R22_ins5- | CUAGUCCGUUAUCAACUUGAAAAAGUGGCACCGAGUCGGUGCAAGUCUGUCUGCC | |
| | nick-v1 | UAUUUGAUGCACCGAUU | |
| RNACS5980 | TRAC4_ | GAGAAUCAAAAUCGGUGAAUGUUUUAGAGCUAGAAAUAGCAAGUUAAAAUAAGG | 17135 |
| | P13R10_ins5- | CUAGUCCGUUAUCAACUUGAAAAAGUGGCACCGAGUCGGUGCCUAUUUAACCCAC | |
| | nick-v2 | CGAUUUUGAU | |
| RNACS5981 | TRAC4_ | GAGAAUCAAAAUCGGUGAAUGUUUUAGAGCUAGAAAUAGCAAGUUAAAAUAAGG | 17136 |
| | P12R10_ins5- | CUAGUCCGUUAUCAACUUGAAAAAGUGGCACCGAGUCGGUGCCUAUUUAACCCAC | |
| | nick-v2 | CGAUUUUGA | |
| RNACS5982 | TRAC4_ | GAGAAUCAAAAUCGGUGAAUGUUUUAGAGCUAGAAAUAGCAAGUUAAAAUAAGG | 17137 |
| | P11R10_ins5- | CUAGUCCGUUAUCAACUUGAAAAAGUGGCACCGAGUCGGUGCCUAUUUAACCCAC | |
| | nick-v2 | CGAUUUUG | |
| RNACS5983 | TRAC4_ | GAGAAUCAAAAUCGGUGAAUGUUUUAGAGCUAGAAAUAGCAAGUUAAAAUAAGG | 17138 |
| | P10R10_ins5- | CUAGUCCGUUAUCAACUUGAAAAAGUGGCACCGAGUCGGUGCCUAUUUAACCCAC | |
| | nick-v2 | CGAUUUU | |
| RNACS5984 | TRAC4_ | GAGAAUCAAAAUCGGUGAAUGUUUUAGAGCUAGAAAUAGCAAGUUAAAAUAAGG | 17139 |
| | P9R10_ins5- | CUAGUCCGUUAUCAACUUGAAAAAGUGGCACCGAGUCGGUGCCUAUUUAACCCAC | |
| | nick-v2 | CGAUUU | |
| RNACS5985 | TRAC4_ | GAGAAUCAAAAUCGGUGAAUGUUUUAGAGCUAGAAAUAGCAAGUUAAAAUAAGG | 17140 |
| | P8R10_ins5- | CUAGUCCGUUAUCAACUUGAAAAAGUGGCACCGAGUCGGUGCCUAUUUAACCCAC | |
| | nick-v2 | CGAUU | |
| RNACS5986 | TRAC4_ | GAGAAUCAAAAUCGGUGAAUGUUUUAGAGCUAGAAAUAGCAAGUUAAAAUAAGG | 17141 |
| | P13R12_ins5- | CUAGUCCGUUAUCAACUUGAAAAAGUGGCACCGAGUCGGUGCGCCUAUUUAACCC | |
| | nick-v2 | ACCGAUUUUGAU | |
| RNACS5987 | TRAC4_ | GAGAAUCAAAAUCGGUGAAUGUUUUAGAGCUAGAAAUAGCAAGUUAAAAUAAGG | 17142 |
| | P12R12_ins5- | CUAGUCCGUUAUCAACUUGAAAAAGUGGCACCGAGUCGGUGCGCCUAUUUAACCC | |
| | nick-v2 | ACCGAUUUUGA | |
| RNACS5988 | TRAC4_ | GAGAAUCAAAAUCGGUGAAUGUUUUAGAGCUAGAAAUAGCAAGUUAAAAUAAGG | 17143 |
| | P11R12_ins5- | CUAGUCCGUUAUCAACUUGAAAAAGUGGCACCGAGUCGGUGCGCCUAUUUAACCC | |
| | nick-v2 | ACCGAUUUUG | |
| RNACS5989 | TRAC4_ | GAGAAUCAAAAUCGGUGAAUGUUUUAGAGCUAGAAAUAGCAAGUUAAAAUAAGG | 17144 |
| | P10R12_ins5- | CUAGUCCGUUAUCAACUUGAAAAAGUGGCACCGAGUCGGUGCGCCUAUUUAACCC | |
| | nick-v2 | ACCGAUUUU | |
| RNACS5990 | TRAC4_ | GAGAAUCAAAAUCGGUGAAUGUUUUAGAGCUAGAAAUAGCAAGUUAAAAUAAGG | 17145 |
| | P9R12_ins5- | CUAGUCCGUUAUCAACUUGAAAAAGUGGCACCGAGUCGGUGCGCCUAUUUAACCC | |
| | nick-v2 | ACCGAUUU | |
| RNACS5991 | TRAC4_ | GAGAAUCAAAAUCGGUGAAUGUUUUAGAGCUAGAAAUAGCAAGUUAAAAUAAGG | 17146 |
| | P8R12_ins5- | CUAGUCCGUUAUCAACUUGAAAAAGUGGCACCGAGUCGGUGCGCCUAUUUAACCC | |
| | nick-v2 | ACCGAUU | |
| RNACS5992 | TRAC4_ | GAGAAUCAAAAUCGGUGAAUGUUUUAGAGCUAGAAAUAGCAAGUUAAAAUAAGG | 17147 |
| | P13R14_ins5- | CUAGUCCGUUAUCAACUUGAAAAAGUGGCACCGAGUCGGUGCCUGCCUAUUUAAC | |
| | nick-v2 | CCACCGAUUUUGAU | |
| RNACS5993 | TRAC4_ | GAGAAUCAAAAUCGGUGAAUGUUUUAGAGCUAGAAAUAGCAAGUUAAAAUAAGG | 17148 |
| | P12R14_ins5- | CUAGUCCGUUAUCAACUUGAAAAAGUGGCACCGAGUCGGUGCCUGCCUAUUUAAC | |
| | nick-v2 | CCACCGAUUUUGA | |
| RNACS5994 | TRAC4_ | GAGAAUCAAAAUCGGUGAAUGUUUUAGAGCUAGAAAUAGCAAGUUAAAAUAAGG | 17149 |
| | P11R14_ins5- | CUAGUCCGUUAUCAACUUGAAAAAGUGGCACCGAGUCGGUGCCUGCCUAUUUAAC | |
| | nick-v2 | CCACCGAUUUUG | |
| RNACS5995 | TRAC4_ | GAGAAUCAAAAUCGGUGAAUGUUUUAGAGCUAGAAAUAGCAAGUUAAAAUAAGG | 17150 |
| | P10R14_ins5- | CUAGUCCGUUAUCAACUUGAAAAAGUGGCACCGAGUCGGUGCCUGCCUAUUUAAC | |
| | nick-v2 | CCACCGAUUUU | |
| RNACS5996 | TRAC4_ | GAGAAUCAAAAUCGGUGAAUGUUUUAGAGCUAGAAAUAGCAAGUUAAAAUAAGG | 17151 |
| | P9R14_ins5- | CUAGUCCGUUAUCAACUUGAAAAAGUGGCACCGAGUCGGUGCCUGCCUAUUUAAC | |
| | nick-v2 | CCACCGAUUU | |
| RNACS5997 | TRAC4_ | GAGAAUCAAAAUCGGUGAAUGUUUUAGAGCUAGAAAUAGCAAGUUAAAAUAAGG | 17152 |
| | P8R14_ins5- | CUAGUCCGUUAUCAACUUGAAAAAGUGGCACCGAGUCGGUGCCUGCCUAUUUAAC | |
| | nick-v2 | CCACCGAUU | |
| RNACS5998 | TRAC4_ | GAGAAUCAAAAUCGGUGAAUGUUUUAGAGCUAGAAAUAGCAAGUUAAAAUAAGG | 17153 |
| | P13R16_ins5- | CUAGUCCGUUAUCAACUUGAAAAAGUGGCACCGAGUCGGUGCGUCUGCCUAUUUA | |
| | nick-v2 | ACCCACCGAUUUUGAU | |
| RNACS5999 | TRAC4_ | GAGAAUCAAAAUCGGUGAAUGUUUUAGAGCUAGAAAUAGCAAGUUAAAAUAAGG | 17154 |
| | P12R16_ins5- | CUAGUCCGUUAUCAACUUGAAAAAGUGGCACCGAGUCGGUGCGUCUGCCUAUUUA | |
| | nick-v2 | ACCCACCGAUUUUGA | |
| RNACS6000 | TRAC4_ | GAGAAUCAAAAUCGGUGAAUGUUUUAGAGCUAGAAAUAGCAAGUUAAAAUAAGG | 17155 |
| | P11R16_ins5- | CUAGUCCGUUAUCAACUUGAAAAAGUGGCACCGAGUCGGUGCGUCUGCCUAUUUA | |
| | nick-v2 | ACCCACCGAUUUUG | |
| RNACS6001 | TRAC4_ | GAGAAUCAAAAUCGGUGAAUGUUUUAGAGCUAGAAAUAGCAAGUUAAAAUAAGG | 17156 |
| | P10R16_ins5- | CUAGUCCGUUAUCAACUUGAAAAAGUGGCACCGAGUCGGUGCGUCUGCCUAUUUA | |
| | nick-v2 | ACCCACCGAUUUU | |
| RNACS6002 | TRAC4_ | GAGAAUCAAAAUCGGUGAAUGUUUUAGAGCUAGAAAUAGCAAGUUAAAAUAAGG | 17157 |
| | P9R16_ins5- | CUAGUCCGUUAUCAACUUGAAAAAGUGGCACCGAGUCGGUGCGUCUGCCUAUUUA | |
| | nick-v2 | ACCCACCGAUUU | |
| RNACS6003 | TRAC4_ | GAGAAUCAAAAUCGGUGAAUGUUUUAGAGCUAGAAAUAGCAAGUUAAAAUAAGG | 17158 |
| | P8R16_ins5- | CUAGUCCGUUAUCAACUUGAAAAAGUGGCACCGAGUCGGUGCGUCUGCCUAUUUA | |
| | nick-v2 | ACCCACCGAUU | |
| RNACS6004 | TRAC4_ | GAGAAUCAAAAUCGGUGAAUGUUUUAGAGCUAGAAAUAGCAAGUUAAAAUAAGG | 17159 |
| | P13R18_ins5- | CUAGUCCGUUAUCAACUUGAAAAAGUGGCACCGAGUCGGUGCCUGUCUGCCUAUU | |
| | nick-v2 | UAACCCACCGAUUUUGAU | |
| RNACS6005 | TRAC4_ | GAGAAUCAAAAUCGGUGAAUGUUUUAGAGCUAGAAAUAGCAAGUUAAAAUAAGG | 17160 |
| | P12R18_ins5- | CUAGUCCGUUAUCAACUUGAAAAAGUGGCACCGAGUCGGUGCCUGUCUGCCUAUU | |
| | nick-v2 | UAACCCACCGAUUUUGA | |
| RNACS6006 | TRAC4_ | GAGAAUCAAAAUCGGUGAAUGUUUUAGAGCUAGAAAUAGCAAGUUAAAAUAAGG | 17161 |
| | P11R18_ins5- | CUAGUCCGUUAUCAACUUGAAAAAGUGGCACCGAGUCGGUGCCUGUCUGCCUAUU | |
| | nick-v2 | UAACCCACCGAUUUUG | |
| RNACS6007 | TRAC4_ | GAGAAUCAAAAUCGGUGAAUGUUUUAGAGCUAGAAAUAGCAAGUUAAAAUAAGG | 17162 |
| | P10R18_ins5- | CUAGUCCGUUAUCAACUUGAAAAAGUGGCACCGAGUCGGUGCCUGUCUGCCUAUU | |
| | nick-v2 | UAACCCACCGAUUUU | |
| RNACS6008 | TRAC4_ | GAGAAUCAAAAUCGGUGAAUGUUUUAGAGCUAGAAAUAGCAAGUUAAAAUAAGG | 17163 |
| | P9R18_ins5- | CUAGUCCGUUAUCAACUUGAAAAAGUGGCACCGAGUCGGUGCCUGUCUGCCUAUU | |
| | nick-v2 | UAACCCACCGAUUU | |
| RNACS6009 | TRAC4_ | GAGAAUCAAAAUCGGUGAAUGUUUUAGAGCUAGAAAUAGCAAGUUAAAAUAAGG | 17164 |
| | P8R18_ins5- | CUAGUCCGUUAUCAACUUGAAAAAGUGGCACCGAGUCGGUGCCUGUCUGCCUAUU | |
| | nick-v2 | UAACCCACCGAUU | |
| RNACS6010 | TRAC4_ | GAGAAUCAAAAUCGGUGAAUGUUUUAGAGCUAGAAAUAGCAAGUUAAAAUAAGG | 17165 |
| | P13R20_ins5- | CUAGUCCGUUAUCAACUUGAAAAAGUGGCACCGAGUCGGUGCGUCUGUCUGCCUA | |
| | nick-v2 | UUUAACCCACCGAUUUUGAU | |
| RNACS6011 | TRAC4_ | GAGAAUCAAAAUCGGUGAAUGUUUUAGAGCUAGAAAUAGCAAGUUAAAAUAAGG | 17166 |
| | P12R20_ins5- | CUAGUCCGUUAUCAACUUGAAAAAGUGGCACCGAGUCGGUGCGUCUGUCUGCCUA | |
| | nick-v2 | UUUAACCCACCGAUUUUGA | |
| RNACS6012 | TRAC4_ | GAGAAUCAAAAUCGGUGAAUGUUUUAGAGCUAGAAAUAGCAAGUUAAAAUAAGG | 17167 |
| | P11R20_ins5- | CUAGUCCGUUAUCAACUUGAAAAAGUGGCACCGAGUCGGUGCGUCUGUCUGCCUA | |
| | nick-v2 | UUUAACCCACCGAUUUUG | |
| RNACS6013 | TRAC4_ | GAGAAUCAAAAUCGGUGAAUGUUUUAGAGCUAGAAAUAGCAAGUUAAAAUAAGG | 17168 |
| | P10R20_ins5- | CUAGUCCGUUAUCAACUUGAAAAAGUGGCACCGAGUCGGUGCGUCUGUCUGCCUA | |
| | nick-v2 | UUUAACCCACCGAUUUU | |
| RNACS6014 | TRAC4_ | GAGAAUCAAAAUCGGUGAAUGUUUUAGAGCUAGAAAUAGCAAGUUAAAAUAAGG | 17169 |
| | P9R20_ins5- | CUAGUCCGUUAUCAACUUGAAAAAGUGGCACCGAGUCGGUGCGUCUGUCUGCCUA | |
| | nick-v2 | UUUAACCCACCGAUUU | |
| RNACS6015 | TRAC4_ | GAGAAUCAAAAUCGGUGAAUGUUUUAGAGCUAGAAAUAGCAAGUUAAAAUAAGG | 17170 |
| | P8R20_ins5- | CUAGUCCGUUAUCAACUUGAAAAAGUGGCACCGAGUCGGUGCGUCUGUCUGCCUA | |
| | nick-v2 | UUUAACCCACCGAUU | |
| RNACS6016 | TRAC4_ | GAGAAUCAAAAUCGGUGAAUGUUUUAGAGCUAGAAAUAGCAAGUUAAAAUAAGG | 17171 |
| | P13R22_ins5- | CUAGUCCGUUAUCAACUUGAAAAAGUGGCACCGAGUCGGUGCAAGUCUGUCUGCC | |
| | nick-v2 | UAUUUAACCCACCGAUUUUGAU | |
| RNACS6017 | TRAC4_ | GAGAAUCAAAAUCGGUGAAUGUUUUAGAGCUAGAAAUAGCAAGUUAAAAUAAGG | 17172 |
| | P12R22_ins5- | CUAGUCCGUUAUCAACUUGAAAAAGUGGCACCGAGUCGGUGCAAGUCUGUCUGCC | |
| | nick-v2 | UAUUUAACCCACCGAUUUUGA | |
| RNACS6018 | TRAC4_ | GAGAAUCAAAAUCGGUGAAUGUUUUAGAGCUAGAAAUAGCAAGUUAAAAUAAGG | 17173 |
| | P11R22_ins5- | CUAGUCCGUUAUCAACUUGAAAAAGUGGCACCGAGUCGGUGCAAGUCUGUCUGCC | |
| | nick-v2 | UAUUUAACCCACCGAUUUUG | |
| RNACS6019 | TRAC4_ | GAGAAUCAAAAUCGGUGAAUGUUUUAGAGCUAGAAAUAGCAAGUUAAAAUAAGG | 17174 |
| | P10R22_ins5- | CUAGUCCGUUAUCAACUUGAAAAAGUGGCACCGAGUCGGUGCAAGUCUGUCUGCC | |
| | nick-v2 | UAUUUAACCCACCGAUUUU | |
| RNACS6020 | TRAC4_ | GAGAAUCAAAAUCGGUGAAUGUUUUAGAGCUAGAAAUAGCAAGUUAAAAUAAGG | 17175 |
| | P9R22_ins5- | CUAGUCCGUUAUCAACUUGAAAAAGUGGCACCGAGUCGGUGCAAGUCUGUCUGCC | |
| | nick-v2 | UAUUUAACCCACCGAUUU | |
| RNACS6021 | TRAC4_ | GAGAAUCAAAAUCGGUGAAUGUUUUAGAGCUAGAAAUAGCAAGUUAAAAUAAGG | 17176 |
| | P8R22_ins5- | CUAGUCCGUUAUCAACUUGAAAAAGUGGCACCGAGUCGGUGCAAGUCUGUCUGCC | |
| | nick-v2 | UAUUUAACCCACCGAUU | |
| RNACS6022 | TRAC5_ | GCUGGUACACGGCAGGGUCAGUUUUAGAGCUAGAAAUAGCAAGUUAAAAUAAGG | 17177 |
| | P13R10_ins5- | CUAGUCCGUUAUCAACUUGAAAAAGUGGCACCGAGUCGGUGCCCUGAUAGUGCCC | |
| | nick | UGCCGUGUAC | |
| RNACS6023 | TRAC5_ | GCUGGUACACGGCAGGGUCAGUUUUAGAGCUAGAAAUAGCAAGUUAAAAUAAGG | 17178 |
| | P12R10_ins5- | CUAGUCCGUUAUCAACUUGAAAAAGUGGCACCGAGUCGGUGCCCUGAUAGUGCCC | |
| | nick | UGCCGUGUA | |
| RNACS6024 | TRAC5_ | GCUGGUACACGGCAGGGUCAGUUUUAGAGCUAGAAAUAGCAAGUUAAAAUAAGG | 17179 |
| | P11R10_ins5- | CUAGUCCGUUAUCAACUUGAAAAAGUGGCACCGAGUCGGUGCCCUGAUAGUGCCC | |
| | nick | UGCCGUGU | |
| RNACS6025 | TRAC5_ | GCUGGUACACGGCAGGGUCAGUUUUAGAGCUAGAAAUAGCAAGUUAAAAUAAGG | 17180 |
| | P10R10_ins5- | CUAGUCCGUUAUCAACUUGAAAAAGUGGCACCGAGUCGGUGCCCUGAUAGUGCCC | |
| | nick | UGCCGUG | |
| RNACS6026 | TRAC5_ | GCUGGUACACGGCAGGGUCAGUUUUAGAGCUAGAAAUAGCAAGUUAAAAUAAGG | 17181 |
| | P9R10_ins5- | CUAGUCCGUUAUCAACUUGAAAAAGUGGCACCGAGUCGGUGCCCUGAUAGUGCCC | |
| | nick | UGCCGU | |
| RNACS6027 | TRAC5_ | GCUGGUACACGGCAGGGUCAGUUUUAGAGCUAGAAAUAGCAAGUUAAAAUAAGG | 17182 |
| | P8R10_ins5- | CUAGUCCGUUAUCAACUUGAAAAAGUGGCACCGAGUCGGUGCCCUGAUAGUGCCC | |
| | nick | UGCCG | |
| RNACS6028 | TRAC5_ | GCUGGUACACGGCAGGGUCAGUUUUAGAGCUAGAAAUAGCAAGUUAAAAUAAGG | 17183 |
| | P13R12_ins5- | CUAGUCCGUUAUCAACUUGAAAAAGUGGCACCGAGUCGGUGCACCCUGAUAGUGC | |
| | nick | CCUGCCGUGUAC | |
| RNACS6029 | TRAC5_ | GCUGGUACACGGCAGGGUCAGUUUUAGAGCUAGAAAUAGCAAGUUAAAAUAAGG | 17184 |
| | P12R12_ins5- | CUAGUCCGUUAUCAACUUGAAAAAGUGGCACCGAGUCGGUGCACCCUGAUAGUGC | |
| | nick | CCUGCCGUGUA | |
| RNACS6030 | TRAC5_ | GCUGGUACACGGCAGGGUCAGUUUUAGAGCUAGAAAUAGCAAGUUAAAAUAAGG | 17185 |
| | P11R12_ins5- | CUAGUCCGUUAUCAACUUGAAAAAGUGGCACCGAGUCGGUGCACCCUGAUAGUGC | |
| | nick | CCUGCCGUGU | |
| RNACS6031 | TRAC5_ | GCUGGUACACGGCAGGGUCAGUUUUAGAGCUAGAAAUAGCAAGUUAAAAUAAGG | 17186 |
| | P10R12_ins5- | CUAGUCCGUUAUCAACUUGAAAAAGUGGCACCGAGUCGGUGCACCCUGAUAGUGC | |
| | nick | CCUGCCGUG | |
| RNACS6032 | TRAC5_ | GCUGGUACACGGCAGGGUCAGUUUUAGAGCUAGAAAUAGCAAGUUAAAAUAAGG | 17187 |
| | P9R12_ins5- | CUAGUCCGUUAUCAACUUGAAAAAGUGGCACCGAGUCGGUGCACCCUGAUAGUGC | |
| | nick | CCUGCCGU | |
| RNACS6033 | TRAC5_ | GCUGGUACACGGCAGGGUCAGUUUUAGAGCUAGAAAUAGCAAGUUAAAAUAAGG | 17188 |
| | P8R12_ins5- | CUAGUCCGUUAUCAACUUGAAAAAGUGGCACCGAGUCGGUGCACCCUGAUAGUGC | |
| | nick | CCUGCCG | |
| RNACS6034 | TRAC5_ | GCUGGUACACGGCAGGGUCAGUUUUAGAGCUAGAAAUAGCAAGUUAAAAUAAGG | 17189 |
| | P13R14_ins5- | CUAGUCCGUUAUCAACUUGAAAAAGUGGCACCGAGUCGGUGCGAACCCUGAUAGU | |
| | nick | GCCCUGCCGUGUAC | |
| RNACS6035 | TRAC5_ | GCUGGUACACGGCAGGGUCAGUUUUAGAGCUAGAAAUAGCAAGUUAAAAUAAGG | 17190 |
| | P12R14_ins5- | CUAGUCCGUUAUCAACUUGAAAAAGUGGCACCGAGUCGGUGCGAACCCUGAUAGU | |
| | nick | GCCCUGCCGUGUA | |
| RNACS6036 | TRAC5_ | GCUGGUACACGGCAGGGUCAGUUUUAGAGCUAGAAAUAGCAAGUUAAAAUAAGG | 17191 |
| | P11R14_ins5- | CUAGUCCGUUAUCAACUUGAAAAAGUGGCACCGAGUCGGUGCGAACCCUGAUAGU | |
| | nick | GCCCUGCCGUGU | |
| RNACS6037 | TRAC5_ | GCUGGUACACGGCAGGGUCAGUUUUAGAGCUAGAAAUAGCAAGUUAAAAUAAGG | 17192 |
| | P10R14_ins5- | CUAGUCCGUUAUCAACUUGAAAAAGUGGCACCGAGUCGGUGCGAACCCUGAUAGU | |
| | nick | GCCCUGCCGUG | |
| RNACS6038 | TRAC5_ | GCUGGUACACGGCAGGGUCAGUUUUAGAGCUAGAAAUAGCAAGUUAAAAUAAGG | 17193 |
| | P9R14_ins5- | CUAGUCCGUUAUCAACUUGAAAAAGUGGCACCGAGUCGGUGCGAACCCUGAUAGU | |
| | nick | GCCCUGCCGU | |
| RNACS6039 | TRAC5_ | GCUGGUACACGGCAGGGUCAGUUUUAGAGCUAGAAAUAGCAAGUUAAAAUAAGG | 17194 |
| | P8R14_ins5- | CUAGUCCGUUAUCAACUUGAAAAAGUGGCACCGAGUCGGUGCGAACCCUGAUAGU | |
| | nick | GCCCUGCCG | |
| RNACS6040 | TRAC5_ | GCUGGUACACGGCAGGGUCAGUUUUAGAGCUAGAAAUAGCAAGUUAAAAUAAGG | 17195 |
| | P13R16_ins5- | CUAGUCCGUUAUCAACUUGAAAAAGUGGCACCGAGUCGGUGCCAGAACCCUGAUA | |
| | nick | GUGCCCUGCCGUGUAC | |
| RNACS6041 | TRAC5_ | GCUGGUACACGGCAGGGUCAGUUUUAGAGCUAGAAAUAGCAAGUUAAAAUAAGG | 17196 |
| | P12R16_ins5- | CUAGUCCGUUAUCAACUUGAAAAAGUGGCACCGAGUCGGUGCCAGAACCCUGAUA | |
| | nick | GUGCCCUGCCGUGUA | |
| RNACS6042 | TRAC5_ | GCUGGUACACGGCAGGGUCAGUUUUAGAGCUAGAAAUAGCAAGUUAAAAUAAGG | 17197 |
| | P11R16_ins5- | CUAGUCCGUUAUCAACUUGAAAAAGUGGCACCGAGUCGGUGCCAGAACCCUGAUA | |
| | nick | GUGCCCUGCCGUGU | |
| RNACS6043 | TRAC5_ | GCUGGUACACGGCAGGGUCAGUUUUAGAGCUAGAAAUAGCAAGUUAAAAUAAGG | 17198 |
| | P10R16_ins5- | CUAGUCCGUUAUCAACUUGAAAAAGUGGCACCGAGUCGGUGCCAGAACCCUGAUA | |
| | nick | GUGCCCUGCCGUG | |
| RNACS6044 | TRAC5_ | GCUGGUACACGGCAGGGUCAGUUUUAGAGCUAGAAAUAGCAAGUUAAAAUAAGG | 17199 |
| | P9R16_ins5- | CUAGUCCGUUAUCAACUUGAAAAAGUGGCACCGAGUCGGUGCCAGAACCCUGAUA | |
| | nick | GUGCCCUGCCGU | |
| RNACS6045 | TRAC5_ | GCUGGUACACGGCAGGGUCAGUUUUAGAGCUAGAAAUAGCAAGUUAAAAUAAGG | 17200 |
| | P8R16_ins5- | CUAGUCCGUUAUCAACUUGAAAAAGUGGCACCGAGUCGGUGCCAGAACCCUGAUA | |
| | nick | GUGCCCUGCCG | |
| RNACS6046 | TRAC5_ | GCUGGUACACGGCAGGGUCAGUUUUAGAGCUAGAAAUAGCAAGUUAAAAUAAGG | 17201 |
| | P13R18_ins5- | CUAGUCCGUUAUCAACUUGAAAAAGUGGCACCGAGUCGGUGCUCCAGAACCCUGA | |
| | nick | UAGUGCCCUGCCGUGUAC | |
| RNACS6047 | TRAC5_ | GCUGGUACACGGCAGGGUCAGUUUUAGAGCUAGAAAUAGCAAGUUAAAAUAAGG | 17202 |
| | P12R18_ins5- | CUAGUCCGUUAUCAACUUGAAAAAGUGGCACCGAGUCGGUGCUCCAGAACCCUGA | |
| | nick | UAGUGCCCUGCCGUGUA | |
| RNACS6048 | TRAC5_ | GCUGGUACACGGCAGGGUCAGUUUUAGAGCUAGAAAUAGCAAGUUAAAAUAAGG | 17203 |
| | P11R18_ins5- | CUAGUCCGUUAUCAACUUGAAAAAGUGGCACCGAGUCGGUGCUCCAGAACCCUGA | |
| | nick | UAGUGCCCUGCCGUGU | |
| RNACS6049 | TRAC5_ | GCUGGUACACGGCAGGGUCAGUUUUAGAGCUAGAAAUAGCAAGUUAAAAUAAGG | 17204 |
| | P10R18_ins5- | CUAGUCCGUUAUCAACUUGAAAAAGUGGCACCGAGUCGGUGCUCCAGAACCCUGA | |
| | nick | UAGUGCCCUGCCGUG | |
| RNACS6050 | TRAC5_ | GCUGGUACACGGCAGGGUCAGUUUUAGAGCUAGAAAUAGCAAGUUAAAAUAAGG | 17205 |
| | P9R18_ins5- | CUAGUCCGUUAUCAACUUGAAAAAGUGGCACCGAGUCGGUGCUCCAGAACCCUGA | |
| | nick | UAGUGCCCUGCCGU | |
| RNACS6051 | TRAC5_ | GCUGGUACACGGCAGGGUCAGUUUUAGAGCUAGAAAUAGCAAGUUAAAAUAAGG | 17206 |
| | P8R18_ins5- | CUAGUCCGUUAUCAACUUGAAAAAGUGGCACCGAGUCGGUGCUCCAGAACCCUGA | |
| | nick | UAGUGCCCUGCCG | |
| RNACS6052 | TRAC5_ | GCUGGUACACGGCAGGGUCAGUUUUAGAGCUAGAAAUAGCAAGUUAAAAUAAGG | 17207 |
| | P13R20_ins5- | CUAGUCCGUUAUCAACUUGAAAAAGUGGCACCGAGUCGGUGCUAUCCAGAACCCU | |
| | nick | GAUAGUGCCCUGCCGUGUAC | |
| RNACS6053 | TRAC5_ | GCUGGUACACGGCAGGGUCAGUUUUAGAGCUAGAAAUAGCAAGUUAAAAUAAGG | 17208 |
| | P12R20_ins5- | CUAGUCCGUUAUCAACUUGAAAAAGUGGCACCGAGUCGGUGCUAUCCAGAACCCU | |
| | nick | GAUAGUGCCCUGCCGUGUA | |
| RNACS6054 | TRAC5_ | GCUGGUACACGGCAGGGUCAGUUUUAGAGCUAGAAAUAGCAAGUUAAAAUAAGG | 17209 |
| | P11R20_ins5- | CUAGUCCGUUAUCAACUUGAAAAAGUGGCACCGAGUCGGUGCUAUCCAGAACCCU | |
| | nick | GAUAGUGCCCUGCCGUGU | |
| RNACS6055 | TRAC5_ | GCUGGUACACGGCAGGGUCAGUUUUAGAGCUAGAAAUAGCAAGUUAAAAUAAGG | 17210 |
| | P10R20_ins5- | CUAGUCCGUUAUCAACUUGAAAAAGUGGCACCGAGUCGGUGCUAUCCAGAACCCU | |
| | nick | GAUAGUGCCCUGCCGUG | |
| RNACS6056 | TRAC5_ | GCUGGUACACGGCAGGGUCAGUUUUAGAGCUAGAAAUAGCAAGUUAAAAUAAGG | 17211 |
| | P9R20_ins5- | CUAGUCCGUUAUCAACUUGAAAAAGUGGCACCGAGUCGGUGCUAUCCAGAACCCU | |
| | nick | GAUAGUGCCCUGCCGU | |
| RNACS6057 | TRAC5_ | GCUGGUACACGGCAGGGUCAGUUUUAGAGCUAGAAAUAGCAAGUUAAAAUAAGG | 17212 |
| | P8R20_ins5- | CUAGUCCGUUAUCAACUUGAAAAAGUGGCACCGAGUCGGUGCUAUCCAGAACCCU | |
| | nick | GAUAGUGCCCUGCCG | |
| RNACS6058 | TRAC5_ | GCUGGUACACGGCAGGGUCAGUUUUAGAGCUAGAAAUAGCAAGUUAAAAUAAGG | 17213 |
| | P13R22_ins5- | CUAGUCCGUUAUCAACUUGAAAAAGUGGCACCGAGUCGGUGCGAUAUCCAGAACC | |
| | nick | CUGAUAGUGCCCUGCCGUGUAC | |
| RNACS6059 | TRAC5_ | GCUGGUACACGGCAGGGUCAGUUUUAGAGCUAGAAAUAGCAAGUUAAAAUAAGG | 17214 |
| | P12R22_ins5- | CUAGUCCGUUAUCAACUUGAAAAAGUGGCACCGAGUCGGUGCGAUAUCCAGAACC | |
| | nick | CUGAUAGUGCCCUGCCGUGUA | |
| RNACS6060 | TRAC5_ | GCUGGUACACGGCAGGGUCAGUUUUAGAGCUAGAAAUAGCAAGUUAAAAUAAGG | 17215 |
| | P11R22_ins5- | CUAGUCCGUUAUCAACUUGAAAAAGUGGCACCGAGUCGGUGCGAUAUCCAGAACC | |
| | nick | CUGAUAGUGCCCUGCCGUGU | |
| RNACS6061 | TRAC5_ | GCUGGUACACGGCAGGGUCAGUUUUAGAGCUAGAAAUAGCAAGUUAAAAUAAGG | 17216 |
| | P10R22_ins5- | CUAGUCCGUUAUCAACUUGAAAAAGUGGCACCGAGUCGGUGCGAUAUCCAGAACC | |
| | nick | CUGAUAGUGCCCUGCCGUG | |
| RNACS6062 | TRAC5_ | GCUGGUACACGGCAGGGUCAGUUUUAGAGCUAGAAAUAGCAAGUUAAAAUAAGG | 17217 |
| | P9R22_ins5- | CUAGUCCGUUAUCAACUUGAAAAAGUGGCACCGAGUCGGUGCGAUAUCCAGAACC | |
| | nick | CUGAUAGUGCCCUGCCGU | |
| RNACS6063 | TRAC5_ | GCUGGUACACGGCAGGGUCAGUUUUAGAGCUAGAAAUAGCAAGUUAAAAUAAGG | 17218 |
| | P8R22_ins5- | CUAGUCCGUUAUCAACUUGAAAAAGUGGCACCGAGUCGGUGCGAUAUCCAGAACC | |
| | nick | CUGAUAGUGCCCUGCCG | |
| RNACS6064 | TRAC5_ | GCUGGUACACGGCAGGGUCAGUUUUAGAGCUAGAAAUAGCAAGUUAAAAUAAGG | 17219 |
| | P13R12_ins7- | CUAGUCCGUUAUCAACUUGAAAAAGUGGCACCGAGUCGGUGCCCUGAUAGUUGAC | |
| | nick | CCUGCCGUGUAC | |
| RNACS6065 | TRAC5_ | GCUGGUACACGGCAGGGUCAGUUUUAGAGCUAGAAAUAGCAAGUUAAAAUAAGG | 17220 |
| | P12R12_ins7- | CUAGUCCGUUAUCAACUUGAAAAAGUGGCACCGAGUCGGUGCCCUGAUAGUUGAC | |
| | nick | CCUGCCGUGUA | |
| RNACS6066 | TRAC5_ | GCUGGUACACGGCAGGGUCAGUUUUAGAGCUAGAAAUAGCAAGUUAAAAUAAGG | 17221 |
| | P11R12_ins7- | CUAGUCCGUUAUCAACUUGAAAAAGUGGCACCGAGUCGGUGCCCUGAUAGUUGAC | |
| | nick | CCUGCCGUGU | |
| RNACS6067 | TRAC5_ | GCUGGUACACGGCAGGGUCAGUUUUAGAGCUAGAAAUAGCAAGUUAAAAUAAGG | 17222 |
| | P10R12_ins7- | CUAGUCCGUUAUCAACUUGAAAAAGUGGCACCGAGUCGGUGCCCUGAUAGUUGAC | |
| | nick | CCUGCCGUG | |
| RNACS6068 | TRAC5_ | GCUGGUACACGGCAGGGUCAGUUUUAGAGCUAGAAAUAGCAAGUUAAAAUAAGG | 17223 |
| | P9R12_ins7- | CUAGUCCGUUAUCAACUUGAAAAAGUGGCACCGAGUCGGUGCCCUGAUAGUUGAC | |
| | nick | CCUGCCGU | |
| RNACS6069 | TRAC5_ | GCUGGUACACGGCAGGGUCAGUUUUAGAGCUAGAAAUAGCAAGUUAAAAUAAGG | 17224 |
| | P8R12_ins7- | CUAGUCCGUUAUCAACUUGAAAAAGUGGCACCGAGUCGGUGCCCUGAUAGUUGAC | |
| | nick | CCUGCCG | |
| RNACS6070 | TRAC5_ | GCUGGUACACGGCAGGGUCAGUUUUAGAGCUAGAAAUAGCAAGUUAAAAUAAGG | 17225 |
| | P13R14_ins7- | CUAGUCCGUUAUCAACUUGAAAAAGUGGCACCGAGUCGGUGCACCCUGAUAGUUG | |
| | nick | ACCCUGCCGUGUAC | |
| RNACS6071 | TRAC5_ | GCUGGUACACGGCAGGGUCAGUUUUAGAGCUAGAAAUAGCAAGUUAAAAUAAGG | 17226 |
| | P12R14_ins7- | CUAGUCCGUUAUCAACUUGAAAAAGUGGCACCGAGUCGGUGCACCCUGAUAGUUG | |
| | nick | ACCCUGCCGUGUA | |
| RNACS6072 | TRAC5_ | GCUGGUACACGGCAGGGUCAGUUUUAGAGCUAGAAAUAGCAAGUUAAAAUAAGG | 17227 |
| | P11R14_ins7- | CUAGUCCGUUAUCAACUUGAAAAAGUGGCACCGAGUCGGUGCACCCUGAUAGUUG | |
| | nick | ACCCUGCCGUGU | |
| RNACS6073 | TRAC5_ | GCUGGUACACGGCAGGGUCAGUUUUAGAGCUAGAAAUAGCAAGUUAAAAUAAGG | 17228 |
| | P10R14_ins7- | CUAGUCCGUUAUCAACUUGAAAAAGUGGCACCGAGUCGGUGCACCCUGAUAGUUG | |
| | nick | ACCCUGCCGUG | |
| RNACS6074 | TRAC5_ | GCUGGUACACGGCAGGGUCAGUUUUAGAGCUAGAAAUAGCAAGUUAAAAUAAGG | 17229 |
| | P9R14_ins7- | CUAGUCCGUUAUCAACUUGAAAAAGUGGCACCGAGUCGGUGCACCCUGAUAGUUG | |
| | nick | ACCCUGCCGU | |
| RNACS6075 | TRAC5_ | GCUGGUACACGGCAGGGUCAGUUUUAGAGCUAGAAAUAGCAAGUUAAAAUAAGG | 17230 |
| | P8R14_ins7- | CUAGUCCGUUAUCAACUUGAAAAAGUGGCACCGAGUCGGUGCACCCUGAUAGUUG | |
| | nick | ACCCUGCCG | |
| RNACS6076 | TRAC5_ | GCUGGUACACGGCAGGGUCAGUUUUAGAGCUAGAAAUAGCAAGUUAAAAUAAGG | 17231 |
| | P13R16_ins7- | CUAGUCCGUUAUCAACUUGAAAAAGUGGCACCGAGUCGGUGCGAACCCUGAUAGU | |
| | nick | UGACCCUGCCGUGUAC | |
| RNACS6077 | TRAC5_ | GCUGGUACACGGCAGGGUCAGUUUUAGAGCUAGAAAUAGCAAGUUAAAAUAAGG | 17232 |
| | P12R16_ins7- | CUAGUCCGUUAUCAACUUGAAAAAGUGGCACCGAGUCGGUGCGAACCCUGAUAGU | |
| | nick | UGACCCUGCCGUGUA | |
| RNACS6078 | TRAC5_ | GCUGGUACACGGCAGGGUCAGUUUUAGAGCUAGAAAUAGCAAGUUAAAAUAAGG | 17233 |
| | P11R16_ins7- | CUAGUCCGUUAUCAACUUGAAAAAGUGGCACCGAGUCGGUGCGAACCCUGAUAGU | |
| | nick | UGACCCUGCCGUGU | |
| RNACS6079 | TRAC5_ | GCUGGUACACGGCAGGGUCAGUUUUAGAGCUAGAAAUAGCAAGUUAAAAUAAGG | 17234 |
| | P10R16_ins7- | CUAGUCCGUUAUCAACUUGAAAAAGUGGCACCGAGUCGGUGCGAACCCUGAUAGU | |
| | nick | UGACCCUGCCGUG | |
| RNACS6080 | TRAC5_ | GCUGGUACACGGCAGGGUCAGUUUUAGAGCUAGAAAUAGCAAGUUAAAAUAAGG | 17235 |
| | P9R16_ins7- | CUAGUCCGUUAUCAACUUGAAAAAGUGGCACCGAGUCGGUGCGAACCCUGAUAGU | |
| | nick | UGACCCUGCCGU | |
| RNACS6081 | TRAC5_ | GCUGGUACACGGCAGGGUCAGUUUUAGAGCUAGAAAUAGCAAGUUAAAAUAAGG | 17236 |
| | P8R16_ins7- | CUAGUCCGUUAUCAACUUGAAAAAGUGGCACCGAGUCGGUGCGAACCCUGAUAGU | |
| | nick | UGACCCUGCCG | |
| RNACS6082 | TRAC5_ | GCUGGUACACGGCAGGGUCAGUUUUAGAGCUAGAAAUAGCAAGUUAAAAUAAGG | 17237 |
| | P13R18_ins7- | CUAGUCCGUUAUCAACUUGAAAAAGUGGCACCGAGUCGGUGCCAGAACCCUGAUA | |
| | nick | GUUGACCCUGCCGUGUAC | |
| RNACS6083 | TRAC5_ | GCUGGUACACGGCAGGGUCAGUUUUAGAGCUAGAAAUAGCAAGUUAAAAUAAGG | 17238 |
| | P12R18_ins7- | CUAGUCCGUUAUCAACUUGAAAAAGUGGCACCGAGUCGGUGCCAGAACCCUGAUA | |
| | nick | GUUGACCCUGCCGUGUA | |
| RNACS6084 | TRAC5_ | GCUGGUACACGGCAGGGUCAGUUUUAGAGCUAGAAAUAGCAAGUUAAAAUAAGG | 17239 |
| | P11R18_ins7- | CUAGUCCGUUAUCAACUUGAAAAAGUGGCACCGAGUCGGUGCCAGAACCCUGAUA | |
| | nick | GUUGACCCUGCCGUGU | |
| RNACS6085 | TRAC5_ | GCUGGUACACGGCAGGGUCAGUUUUAGAGCUAGAAAUAGCAAGUUAAAAUAAGG | 17240 |
| | P10R18_ins7- | CUAGUCCGUUAUCAACUUGAAAAAGUGGCACCGAGUCGGUGCCAGAACCCUGAUA | |
| | nick | GUUGACCCUGCCGUG | |
| RNACS6086 | TRAC5_ | GCUGGUACACGGCAGGGUCAGUUUUAGAGCUAGAAAUAGCAAGUUAAAAUAAGG | 17241 |
| | P9R18_ins7- | CUAGUCCGUUAUCAACUUGAAAAAGUGGCACCGAGUCGGUGCCAGAACCCUGAUA | |
| | nick | GUUGACCCUGCCGU | |
| RNACS6087 | TRAC5_ | GCUGGUACACGGCAGGGUCAGUUUUAGAGCUAGAAAUAGCAAGUUAAAAUAAGG | 17242 |
| | P8R18_ins7- | CUAGUCCGUUAUCAACUUGAAAAAGUGGCACCGAGUCGGUGCCAGAACCCUGAUA | |
| | nick | GUUGACCCUGCCG | |
| RNACS6088 | TRAC5_ | GCUGGUACACGGCAGGGUCAGUUUUAGAGCUAGAAAUAGCAAGUUAAAAUAAGG | 17243 |
| | P13R20_ins7- | CUAGUCCGUUAUCAACUUGAAAAAGUGGCACCGAGUCGGUGCUCCAGAACCCUGA | |
| | nick | UAGUUGACCCUGCCGUGUAC | |
| RNACS6089 | TRAC5_ | GCUGGUACACGGCAGGGUCAGUUUUAGAGCUAGAAAUAGCAAGUUAAAAUAAGG | 17244 |
| | P12R20_ins7- | CUAGUCCGUUAUCAACUUGAAAAAGUGGCACCGAGUCGGUGCUCCAGAACCCUGA | |
| | nick | UAGUUGACCCUGCCGUGUA | |
| RNACS6090 | TRAC5_ | GCUGGUACACGGCAGGGUCAGUUUUAGAGCUAGAAAUAGCAAGUUAAAAUAAGG | 17245 |
| | P11R20_ins7- | CUAGUCCGUUAUCAACUUGAAAAAGUGGCACCGAGUCGGUGCUCCAGAACCCUGA | |
| | nick | UAGUUGACCCUGCCGUGU | |
| RNACS6091 | TRAC5_ | GCUGGUACACGGCAGGGUCAGUUUUAGAGCUAGAAAUAGCAAGUUAAAAUAAGG | 17246 |
| | P10R20_ins7- | CUAGUCCGUUAUCAACUUGAAAAAGUGGCACCGAGUCGGUGCUCCAGAACCCUGA | |
| | nick | UAGUUGACCCUGCCGUG | |
| RNACS6092 | TRAC5_ | GCUGGUACACGGCAGGGUCAGUUUUAGAGCUAGAAAUAGCAAGUUAAAAUAAGG | 17247 |
| | P9R20_ins7- | CUAGUCCGUUAUCAACUUGAAAAAGUGGCACCGAGUCGGUGCUCCAGAACCCUGA | |
| | nick | UAGUUGACCCUGCCGU | |
| RNACS6093 | TRAC5_ | GCUGGUACACGGCAGGGUCAGUUUUAGAGCUAGAAAUAGCAAGUUAAAAUAAGG | 17248 |
| | P8R20_ins7- | CUAGUCCGUUAUCAACUUGAAAAAGUGGCACCGAGUCGGUGCUCCAGAACCCUGA | |
| | nick | UAGUUGACCCUGCCG | |
| RNACS6094 | TRAC5_ | GCUGGUACACGGCAGGGUCAGUUUUAGAGCUAGAAAUAGCAAGUUAAAAUAAGG | 17249 |
| | P13R22_ins7- | CUAGUCCGUUAUCAACUUGAAAAAGUGGCACCGAGUCGGUGCUAUCCAGAACCCU | |
| | nick | GAUAGUUGACCCUGCCGUGUAC | |
| RNACS6095 | TRAC5_ | GCUGGUACACGGCAGGGUCAGUUUUAGAGCUAGAAAUAGCAAGUUAAAAUAAGG | 17250 |
| | P12R22_ins7- | CUAGUCCGUUAUCAACUUGAAAAAGUGGCACCGAGUCGGUGCUAUCCAGAACCCU | |
| | nick | GAUAGUUGACCCUGCCGUGUA | |
| RNACS6096 | TRAC5_ | GCUGGUACACGGCAGGGUCAGUUUUAGAGCUAGAAAUAGCAAGUUAAAAUAAGG | 17251 |
| | P11R22_ins7- | CUAGUCCGUUAUCAACUUGAAAAAGUGGCACCGAGUCGGUGCUAUCCAGAACCCU | |
| | nick | GAUAGUUGACCCUGCCGUGU | |
| RNACS6097 | TRAC5_ | GCUGGUACACGGCAGGGUCAGUUUUAGAGCUAGAAAUAGCAAGUUAAAAUAAGG | 17252 |
| | P10R22_ins7- | CUAGUCCGUUAUCAACUUGAAAAAGUGGCACCGAGUCGGUGCUAUCCAGAACCCU | |
| | nick | GAUAGUUGACCCUGCCGUG | |
| RNACS6098 | TRAC5_ | GCUGGUACACGGCAGGGUCAGUUUUAGAGCUAGAAAUAGCAAGUUAAAAUAAGG | 17253 |
| | P9R22_ins7- | CUAGUCCGUUAUCAACUUGAAAAAGUGGCACCGAGUCGGUGCUAUCCAGAACCCU | |
| | nick | GAUAGUUGACCCUGCCGU | |
| RNACS6099 | TRAC5_ | GCUGGUACACGGCAGGGUCAGUUUUAGAGCUAGAAAUAGCAAGUUAAAAUAAGG | 17254 |
| | P8R22_ins7- | CUAGUCCGUUAUCAACUUGAAAAAGUGGCACCGAGUCGGUGCUAUCCAGAACCCU | |
| | nick | GAUAGUUGACCCUGCCG | |
| RNACS6100 | TRAC5_ | GCUGGUACACGGCAGGGUCAGUUUUAGAGCUAGAAAUAGCAAGUUAAAAUAAGG | 17255 |
| | P13R24_ins7- | CUAGUCCGUUAUCAACUUGAAAAAGUGGCACCGAGUCGGUGCGAUAUCCAGAACC | |
| | nick | CUGAUAGUUGACCCUGCCGUGUAC | |
| RNACS6101 | TRAC5_ | GCUGGUACACGGCAGGGUCAGUUUUAGAGCUAGAAAUAGCAAGUUAAAAUAAGG | 17256 |
| | P12R24_ins7- | CUAGUCCGUUAUCAACUUGAAAAAGUGGCACCGAGUCGGUGCGAUAUCCAGAACC | |
| | nick | CUGAUAGUUGACCCUGCCGUGUA | |
| RNACS6102 | TRAC5_ | GCUGGUACACGGCAGGGUCAGUUUUAGAGCUAGAAAUAGCAAGUUAAAAUAAGG | 17257 |
| | P11R24_ins7- | CUAGUCCGUUAUCAACUUGAAAAAGUGGCACCGAGUCGGUGCGAUAUCCAGAACC | |
| | nick | CUGAUAGUUGACCCUGCCGUGU | |
| RNACS6103 | TRAC5_ | GCUGGUACACGGCAGGGUCAGUUUUAGAGCUAGAAAUAGCAAGUUAAAAUAAGG | 17258 |
| | P10R24_ins7- | CUAGUCCGUUAUCAACUUGAAAAAGUGGCACCGAGUCGGUGCGAUAUCCAGAACC | |
| | nick | CUGAUAGUUGACCCUGCCGUG | |
| RNACS6104 | TRAC5_ | GCUGGUACACGGCAGGGUCAGUUUUAGAGCUAGAAAUAGCAAGUUAAAAUAAGG | 17259 |
| | P9R24_ins7- | CUAGUCCGUUAUCAACUUGAAAAAGUGGCACCGAGUCGGUGCGAUAUCCAGAACC | |
| | nick | CUGAUAGUUGACCCUGCCGU | |
| RNACS6105 | TRAC5_ | GCUGGUACACGGCAGGGUCAGUUUUAGAGCUAGAAAUAGCAAGUUAAAAUAAGG | 17260 |
| | P8R24_ins7- | CUAGUCCGUUAUCAACUUGAAAAAGUGGCACCGAGUCGGUGCGAUAUCCAGAACC | |
| | nick | CUGAUAGUUGACCCUGCCG | |
| RNACS6106 | TRAC5_ | GCUGGUACACGGCAGGGUCAGUUUUAGAGCUAGAAAUAGCAAGUUAAAAUAAGG | 17261 |
| | P13R5_del5- | CUAGUCCGUUAUCAACUUGAAAAAGUGGCACCGAGUCGGUGCAGAACCCCUGCCG | |
| | nick | UGUAC | |
| RNACS6107 | TRAC5_ | GCUGGUACACGGCAGGGUCAGUUUUAGAGCUAGAAAUAGCAAGUUAAAAUAAGG | 17262 |
| | P12R5_del5- | CUAGUCCGUUAUCAACUUGAAAAAGUGGCACCGAGUCGGUGCAGAACCCCUGCCG | |
| | nick | UGUA | |
| RNACS6108 | TRAC5_ | GCUGGUACACGGCAGGGUCAGUUUUAGAGCUAGAAAUAGCAAGUUAAAAUAAGG | 17263 |
| | P11R5 de15- | CUAGUCCGUUAUCAACUUGAAAAAGUGGCACCGAGUCGGUGCAGAACCCCUGCCG | |
| | nick | UGU | |
| RNACS6109 | TRAC5_ | GCUGGUACACGGCAGGGUCAGUUUUAGAGCUAGAAAUAGCAAGUUAAAAUAAGG | 17264 |
| | P10R5_del5- | CUAGUCCGUUAUCAACUUGAAAAAGUGGCACCGAGUCGGUGCAGAACCCCUGCCG | |
| | nick | UG | |
| RNACS6110 | TRAC5_ | GCUGGUACACGGCAGGGUCAGUUUUAGAGCUAGAAAUAGCAAGUUAAAAUAAGG | 17265 |
| | P9R5_del5- | CUAGUCCGUUAUCAACUUGAAAAAGUGGCACCGAGUCGGUGCAGAACCCCUGCCG | |
| | nick | U | |
| RNACS6111 | TRAC5_ | GCUGGUACACGGCAGGGUCAGUUUUAGAGCUAGAAAUAGCAAGUUAAAAUAAGG | 17266 |
| | P8R5_del5- | CUAGUCCGUUAUCAACUUGAAAAAGUGGCACCGAGUCGGUGCAGAACCCCUGCCG | |
| | nick | | |
| RNACS6112 | TRAC5_ | GCUGGUACACGGCAGGGUCAGUUUUAGAGCUAGAAAUAGCAAGUUAAAAUAAGG | 17267 |
| | P13R7_del5- | CUAGUCCGUUAUCAACUUGAAAAAGUGGCACCGAGUCGGUGCCCAGAACCCCUGC | |
| | nick | CGUGUAC | |
| RNACS6113 | TRAC5_ | GCUGGUACACGGCAGGGUCAGUUUUAGAGCUAGAAAUAGCAAGUUAAAAUAAGG | 17268 |
| | P12R7_del5- | CUAGUCCGUUAUCAACUUGAAAAAGUGGCACCGAGUCGGUGCCCAGAACCCCUGC | |
| | nick | CGUGUA | |
| RNACS6114 | TRAC5_ | GCUGGUACACGGCAGGGUCAGUUUUAGAGCUAGAAAUAGCAAGUUAAAAUAAGG | 17269 |
| | P11R7_del5- | CUAGUCCGUUAUCAACUUGAAAAAGUGGCACCGAGUCGGUGCCCAGAACCCCUGC | |
| | nick | CGUGU | |
| RNACS6115 | TRAC5_ | GCUGGUACACGGCAGGGUCAGUUUUAGAGCUAGAAAUAGCAAGUUAAAAUAAGG | 17270 |
| | P10R7_del5- | CUAGUCCGUUAUCAACUUGAAAAAGUGGCACCGAGUCGGUGCCCAGAACCCCUGC | |
| | nick | CGUG | |
| RNACS6116 | TRAC5_ | GCUGGUACACGGCAGGGUCAGUUUUAGAGCUAGAAAUAGCAAGUUAAAAUAAGG | 17271 |
| | P9R7_del5- | CUAGUCCGUUAUCAACUUGAAAAAGUGGCACCGAGUCGGUGCCCAGAACCCCUGC | |
| | nick | CGU | |
| RNACS6117 | TRAC5_ | GCUGGUACACGGCAGGGUCAGUUUUAGAGCUAGAAAUAGCAAGUUAAAAUAAGG | 17272 |
| | P8R7_del5- | CUAGUCCGUUAUCAACUUGAAAAAGUGGCACCGAGUCGGUGCCCAGAACCCCUGC | |
| | nick | CG | |
| RNACS6118 | TRAC5_ | GCUGGUACACGGCAGGGUCAGUUUUAGAGCUAGAAAUAGCAAGUUAAAAUAAGG | 17273 |
| | P13R9_del5- | CUAGUCCGUUAUCAACUUGAAAAAGUGGCACCGAGUCGGUGCAUCCAGAACCCCU | |
| | nick | GCCGUGUAC | |
| RNACS6119 | TRAC5_ | GCUGGUACACGGCAGGGUCAGUUUUAGAGCUAGAAAUAGCAAGUUAAAAUAAGG | 17274 |
| | P12R9_del5- | CUAGUCCGUUAUCAACUUGAAAAAGUGGCACCGAGUCGGUGCAUCCAGAACCCCU | |
| | nick | GCCGUGUA | |
| RNACS6120 | TRAC5_ | GCUGGUACACGGCAGGGUCAGUUUUAGAGCUAGAAAUAGCAAGUUAAAAUAAGG | 17275 |
| | P11R9 de15- | CUAGUCCGUUAUCAACUUGAAAAAGUGGCACCGAGUCGGUGCAUCCAGAACCCCU | |
| | nick | GCCGUGU | |
| RNACS6121 | TRAC5_ | GCUGGUACACGGCAGGGUCAGUUUUAGAGCUAGAAAUAGCAAGUUAAAAUAAGG | 17276 |
| | P10R9_del5- | CUAGUCCGUUAUCAACUUGAAAAAGUGGCACCGAGUCGGUGCAUCCAGAACCCCU | |
| | nick | GCCGUG | |
| RNACS6122 | TRAC5_ | GCUGGUACACGGCAGGGUCAGUUUUAGAGCUAGAAAUAGCAAGUUAAAAUAAGG | 17277 |
| | P9R9_del5- | CUAGUCCGUUAUCAACUUGAAAAAGUGGCACCGAGUCGGUGCAUCCAGAACCCCU | |
| | nick | GCCGU | |
| RNACS6123 | TRAC5_ | GCUGGUACACGGCAGGGUCAGUUUUAGAGCUAGAAAUAGCAAGUUAAAAUAAGG | 17278 |
| | P8R9_del5- | CUAGUCCGUUAUCAACUUGAAAAAGUGGCACCGAGUCGGUGCAUCCAGAACCCCU | |
| | nick | GCCG | |
| RNACS6124 | TRAC5_ | GCUGGUACACGGCAGGGUCAGUUUUAGAGCUAGAAAUAGCAAGUUAAAAUAAGG | 17279 |
| | P13R11_del5- | CUAGUCCGUUAUCAACUUGAAAAAGUGGCACCGAGUCGGUGCAUAUCCAGAACCC | |
| | nick | CUGCCGUGUAC | |
| RNACS6125 | TRAC5_ | GCUGGUACACGGCAGGGUCAGUUUUAGAGCUAGAAAUAGCAAGUUAAAAUAAGG | 17280 |
| | P12R11_del5- | CUAGUCCGUUAUCAACUUGAAAAAGUGGCACCGAGUCGGUGCAUAUCCAGAACCC | |
| | nick | CUGCCGUGUA | |
| RNACS6126 | TRAC5_ | GCUGGUACACGGCAGGGUCAGUUUUAGAGCUAGAAAUAGCAAGUUAAAAUAAGG | 17281 |
| | P11R11_del5- | CUAGUCCGUUAUCAACUUGAAAAAGUGGCACCGAGUCGGUGCAUAUCCAGAACCC | |
| | nick | CUGCCGUGU | |
| RNACS6127 | TRAC5_ | GCUGGUACACGGCAGGGUCAGUUUUAGAGCUAGAAAUAGCAAGUUAAAAUAAGG | 17282 |
| | P10R11_del5- | CUAGUCCGUUAUCAACUUGAAAAAGUGGCACCGAGUCGGUGCAUAUCCAGAACCC | |
| | nick | CUGCCGUG | |
| RNACS6128 | TRAC5_ | GCUGGUACACGGCAGGGUCAGUUUUAGAGCUAGAAAUAGCAAGUUAAAAUAAGG | 17283 |
| | P9R11_del5- | CUAGUCCGUUAUCAACUUGAAAAAGUGGCACCGAGUCGGUGCAUAUCCAGAACCC | |
| | nick | CUGCCGU | |
| RNACS6129 | TRAC5_ | GCUGGUACACGGCAGGGUCAGUUUUAGAGCUAGAAAUAGCAAGUUAAAAUAAGG | 17284 |
| | P8R11_del5- | CUAGUCCGUUAUCAACUUGAAAAAGUGGCACCGAGUCGGUGCAUAUCCAGAACCC | |
| | nick | CUGCCG | |
| RNACS6130 | TRAC5_ | GCUGGUACACGGCAGGGUCAGUUUUAGAGCUAGAAAUAGCAAGUUAAAAUAAGG | 17285 |
| | P13R13_del5- | CUAGUCCGUUAUCAACUUGAAAAAGUGGCACCGAGUCGGUGCAGAUAUCCAGAAC | |
| | nick | CCCUGCCGUGUAC | |
| RNACS6131 | TRAC5_ | GCUGGUACACGGCAGGGUCAGUUUUAGAGCUAGAAAUAGCAAGUUAAAAUAAGG | 17286 |
| | P12R13_del5- | CUAGUCCGUUAUCAACUUGAAAAAGUGGCACCGAGUCGGUGCAGAUAUCCAGAAC | |
| | nick | CCCUGCCGUGUA | |
| RNACS6132 | TRAC5_ | GCUGGUACACGGCAGGGUCAGUUUUAGAGCUAGAAAUAGCAAGUUAAAAUAAGG | 17287 |
| | P11R13_del5- | CUAGUCCGUUAUCAACUUGAAAAAGUGGCACCGAGUCGGUGCAGAUAUCCAGAAC | |
| | nick | CCCUGCCGUGU | |
| RNACS6133 | TRAC5_ | GCUGGUACACGGCAGGGUCAGUUUUAGAGCUAGAAAUAGCAAGUUAAAAUAAGG | 17288 |
| | P10R13_del5- | CUAGUCCGUUAUCAACUUGAAAAAGUGGCACCGAGUCGGUGCAGAUAUCCAGAAC | |
| | nick | CCCUGCCGUG | |
| RNACS6134 | TRAC5_ | GCUGGUACACGGCAGGGUCAGUUUUAGAGCUAGAAAUAGCAAGUUAAAAUAAGG | 17289 |
| | P9R13_del5- | CUAGUCCGUUAUCAACUUGAAAAAGUGGCACCGAGUCGGUGCAGAUAUCCAGAAC | |
| | nick | CCCUGCCGU | |
| RNACS6135 | TRAC5_ | GCUGGUACACGGCAGGGUCAGUUUUAGAGCUAGAAAUAGCAAGUUAAAAUAAGG | 17290 |
| | P8R13_del5- | CUAGUCCGUUAUCAACUUGAAAAAGUGGCACCGAGUCGGUGCAGAUAUCCAGAAC | |
| | nick | CCCUGCCG | |
| RNACS6136 | TRAC5_ | GCUGGUACACGGCAGGGUCAGUUUUAGAGCUAGAAAUAGCAAGUUAAAAUAAGG | 17291 |
| | P13R15_del5- | CUAGUCCGUUAUCAACUUGAAAAAGUGGCACCGAGUCGGUGCACAGAUAUCCAGA | |
| | nick | ACCCCUGCCGUGUAC | |
| RNACS6137 | TRAC5_ | GCUGGUACACGGCAGGGUCAGUUUUAGAGCUAGAAAUAGCAAGUUAAAAUAAGG | 17292 |
| | P12R15_del5- | CUAGUCCGUUAUCAACUUGAAAAAGUGGCACCGAGUCGGUGCACAGAUAUCCAGA | |
| | nick | ACCCCUGCCGUGUA | |
| RNACS6138 | TRAC5_ | GCUGGUACACGGCAGGGUCAGUUUUAGAGCUAGAAAUAGCAAGUUAAAAUAAGG | 17293 |
| | P11R15_del5- | CUAGUCCGUUAUCAACUUGAAAAAGUGGCACCGAGUCGGUGCACAGAUAUCCAGA | |
| | nick | ACCCCUGCCGUGU | |
| RNACS6139 | TRAC5_ | GCUGGUACACGGCAGGGUCAGUUUUAGAGCUAGAAAUAGCAAGUUAAAAUAAGG | 17294 |
| | P10R15_del5- | CUAGUCCGUUAUCAACUUGAAAAAGUGGCACCGAGUCGGUGCACAGAUAUCCAGA | |
| | nick | ACCCCUGCCGUG | |
| RNACS6140 | TRAC5_ | GCUGGUACACGGCAGGGUCAGUUUUAGAGCUAGAAAUAGCAAGUUAAAAUAAGG | 17295 |
| | P9R15_del5- | CUAGUCCGUUAUCAACUUGAAAAAGUGGCACCGAGUCGGUGCACAGAUAUCCAGA | |
| | nick | ACCCCUGCCGU | |
| RNACS6141 | TRAC5_ | GCUGGUACACGGCAGGGUCAGUUUUAGAGCUAGAAAUAGCAAGUUAAAAUAAGG | 17296 |
| | P8R15_del5- | CUAGUCCGUUAUCAACUUGAAAAAGUGGCACCGAGUCGGUGCACAGAUAUCCAGA | |
| | nick | ACCCCUGCCG | |
| RNACS6142 | TRAC5_ | GCUGGUACACGGCAGGGUCAGUUUUAGAGCUAGAAAUAGCAAGUUAAAAUAAGG | 17297 |
| | P13R17_del5- | CUAGUCCGUUAUCAACUUGAAAAAGUGGCACCGAGUCGGUGCCCACAGAUAUCCA | |
| | nick | GAACCCCUGCCGUGUAC | |
| RNACS6143 | TRAC5_ | GCUGGUACACGGCAGGGUCAGUUUUAGAGCUAGAAAUAGCAAGUUAAAAUAAGG | 17298 |
| | P12R17_del5- | CUAGUCCGUUAUCAACUUGAAAAAGUGGCACCGAGUCGGUGCCCACAGAUAUCCA | |
| | nick | GAACCCCUGCCGUGUA | |
| RNACS6144 | TRAC5_ | GCUGGUACACGGCAGGGUCAGUUUUAGAGCUAGAAAUAGCAAGUUAAAAUAAGG | 17299 |
| | P11R17_del5- | CUAGUCCGUUAUCAACUUGAAAAAGUGGCACCGAGUCGGUGCCCACAGAUAUCCA | |
| | nick | GAACCCCUGCCGUGU | |
| RNACS6145 | TRAC5_ | GCUGGUACACGGCAGGGUCAGUUUUAGAGCUAGAAAUAGCAAGUUAAAAUAAGG | 17300 |
| | P10R17_del5- | CUAGUCCGUUAUCAACUUGAAAAAGUGGCACCGAGUCGGUGCCCACAGAUAUCCA | |
| | nick | GAACCCCUGCCGUG | |
| RNACS6146 | TRAC5_ | GCUGGUACACGGCAGGGUCAGUUUUAGAGCUAGAAAUAGCAAGUUAAAAUAAGG | 17301 |
| | P9R17_del5- | CUAGUCCGUUAUCAACUUGAAAAAGUGGCACCGAGUCGGUGCCCACAGAUAUCCA | |
| | nick | GAACCCCUGCCGU | |
| RNACS6147 | TRAC5_ | GCUGGUACACGGCAGGGUCAGUUUUAGAGCUAGAAAUAGCAAGUUAAAAUAAGG | 17302 |
| | P8R17_del5- | CUAGUCCGUUAUCAACUUGAAAAAGUGGCACCGAGUCGGUGCCCACAGAUAUCCA | |
| | nick | GAACCCCUGCCG | |
| RNACS6148 | TRAC5_ | GCUGGUACACGGCAGGGUCAGUUUUAGAGCUAGAAAUAGCAAGUUAAAAUAAGG | 17303 |
| | P13R5_del7- | CUAGUCCGUUAUCAACUUGAAAAAGUGGCACCGAGUCGGUGCCCAGACCCUGCCG | |
| | nick | UGUAC | |
| RNACS6149 | TRAC5_ | GCUGGUACACGGCAGGGUCAGUUUUAGAGCUAGAAAUAGCAAGUUAAAAUAAGG | 17304 |
| | P12R5_del7- | CUAGUCCGUUAUCAACUUGAAAAAGUGGCACCGAGUCGGUGCCCAGACCCUGCCG | |
| | nick | UGUA | |
| RNACS6150 | TRAC5_ | GCUGGUACACGGCAGGGUCAGUUUUAGAGCUAGAAAUAGCAAGUUAAAAUAAGG | 17305 |
| | P11R5_del7- | CUAGUCCGUUAUCAACUUGAAAAAGUGGCACCGAGUCGGUGCCCAGACCCUGCCG | |
| | nick | UGU | |
| RNACS6151 | TRAC5_ | GCUGGUACACGGCAGGGUCAGUUUUAGAGCUAGAAAUAGCAAGUUAAAAUAAGG | 17306 |
| | P10R5_del7- | CUAGUCCGUUAUCAACUUGAAAAAGUGGCACCGAGUCGGUGCCCAGACCCUGCCG | |
| | nick | UG | |
| RNACS6152 | TRAC5_ | GCUGGUACACGGCAGGGUCAGUUUUAGAGCUAGAAAUAGCAAGUUAAAAUAAGG | 17307 |
| | P9R5_del7- | CUAGUCCGUUAUCAACUUGAAAAAGUGGCACCGAGUCGGUGCCCAGACCCUGCCG | |
| | nick | U | |
| RNACS6153 | TRAC5_ | GCUGGUACACGGCAGGGUCAGUUUUAGAGCUAGAAAUAGCAAGUUAAAAUAAGG | 17308 |
| | P8R5_del7- | CUAGUCCGUUAUCAACUUGAAAAAGUGGCACCGAGUCGGUGCCCAGACCCUGCCG | |
| | nick | | |
| RNACS6154 | TRAC5_ | GCUGGUACACGGCAGGGUCAGUUUUAGAGCUAGAAAUAGCAAGUUAAAAUAAGG | 17309 |
| | P13R7_del7- | CUAGUCCGUUAUCAACUUGAAAAAGUGGCACCGAGUCGGUGCAUCCAGACCCUGC | |
| | nick | CGUGUAC | |
| RNACS6155 | TRAC5_ | GCUGGUACACGGCAGGGUCAGUUUUAGAGCUAGAAAUAGCAAGUUAAAAUAAGG | 17310 |
| | P12R7_del7- | CUAGUCCGUUAUCAACUUGAAAAAGUGGCACCGAGUCGGUGCAUCCAGACCCUGC | |
| | nick | CGUGUA | |
| RNACS6156 | TRAC5_ | GCUGGUACACGGCAGGGUCAGUUUUAGAGCUAGAAAUAGCAAGUUAAAAUAAGG | 17311 |
| | P11R7_del7- | CUAGUCCGUUAUCAACUUGAAAAAGUGGCACCGAGUCGGUGCAUCCAGACCCUGC | |
| | nick | CGUGU | |
| RNACS6157 | TRAC5_ | GCUGGUACACGGCAGGGUCAGUUUUAGAGCUAGAAAUAGCAAGUUAAAAUAAGG | 17312 |
| | P10R7_del7- | CUAGUCCGUUAUCAACUUGAAAAAGUGGCACCGAGUCGGUGCAUCCAGACCCUGC | |
| | nick | CGUC | |
| RNACS6158 | TRAC5_ | GCUGGUACACGGCAGGGUCAGUUUUAGAGCUAGAAAUAGCAAGUUAAAAUAAGG | 17313 |
| | P9R7_del7- | CUAGUCCGUUAUCAACUUGAAAAAGUGGCACCGAGUCGGUGCAUCCAGACCCUGC | |
| | nick | CGU | |
| RNACS6159 | TRAC5_ | GCUGGUACACGGCAGGGUCAGUUUUAGAGCUAGAAAUAGCAAGUUAAAAUAAGG | 17314 |
| | P8R7_del7- | CUAGUCCGUUAUCAACUUGAAAAAGUGGCACCGAGUCGGUGCAUCCAGACCCUGC | |
| | nick | CG | |
| RNACS6160 | TRAC5_ | GCUGGUACACGGCAGGGUCAGUUUUAGAGCUAGAAAUAGCAAGUUAAAAUAAGG | 17315 |
| | P13R9_del7- | CUAGUCCGUUAUCAACUUGAAAAAGUGGCACCGAGUCGGUGCAUAUCCAGACCCU | |
| | nick | GCCGUGUAC | |
| RNACS6161 | TRAC5_ | GCUGGUACACGGCAGGGUCAGUUUUAGAGCUAGAAAUAGCAAGUUAAAAUAAGG | 17316 |
| | P12R9_del7- | CUAGUCCGUUAUCAACUUGAAAAAGUGGCACCGAGUCGGUGCAUAUCCAGACCCU | |
| | nick | GCCGUGUA | |
| RNACS6162 | TRAC5_ | GCUGGUACACGGCAGGGUCAGUUUUAGAGCUAGAAAUAGCAAGUUAAAAUAAGG | 17317 |
| | P11R9_del7- | CUAGUCCGUUAUCAACUUGAAAAAGUGGCACCGAGUCGGUGCAUAUCCAGACCCU | |
| | nick | GCCGUGU | |
| RNACS6163 | TRAC5_ | GCUGGUACACGGCAGGGUCAGUUUUAGAGCUAGAAAUAGCAAGUUAAAAUAAGG | 17318 |
| | P10R9_del7- | CUAGUCCGUUAUCAACUUGAAAAAGUGGCACCGAGUCGGUGCAUAUCCAGACCCU | |
| | nick | GCCGUG | |
| RNACS6164 | TRAC5_ | GCUGGUACACGGCAGGGUCAGUUUUAGAGCUAGAAAUAGCAAGUUAAAAUAAGG | 17319 |
| | P9R9_del7- | CUAGUCCGUUAUCAACUUGAAAAAGUGGCACCGAGUCGGUGCAUAUCCAGACCCU | |
| | nick | GCCGU | |
| RNACS6165 | TRAC5_ | GCUGGUACACGGCAGGGUCAGUUUUAGAGCUAGAAAUAGCAAGUUAAAAUAAGG | 17320 |
| | P8R9_del7- | CUAGUCCGUUAUCAACUUGAAAAAGUGGCACCGAGUCGGUGCAUAUCCAGACCCU | |
| | nick | GCCG | |
| RNACS6166 | TRAC5_ | GCUGGUACACGGCAGGGUCAGUUUUAGAGCUAGAAAUAGCAAGUUAAAAUAAGG | 17321 |
| | P13R11_del7- | CUAGUCCGUUAUCAACUUGAAAAAGUGGCACCGAGUCGGUGCAGAUAUCCAGACC | |
| | nick | CUGCCGUGUAC | |
| RNACS6167 | TRAC5_ | GCUGGUACACGGCAGGGUCAGUUUUAGAGCUAGAAAUAGCAAGUUAAAAUAAGG | 17322 |
| | P12R11_del7- | CUAGUCCGUUAUCAACUUGAAAAAGUGGCACCGAGUCGGUGCAGAUAUCCAGACC | |
| | nick | CUGCCGUGUA | |
| RNACS6168 | TRAC5_ | GCUGGUACACGGCAGGGUCAGUUUUAGAGCUAGAAAUAGCAAGUUAAAAUAAGG | 17323 |
| | P11R11_del7- | CUAGUCCGUUAUCAACUUGAAAAAGUGGCACCGAGUCGGUGCAGAUAUCCAGACC | |
| | nick | CUGCCGUGU | |
| RNACS6169 | TRAC5_ | GCUGGUACACGGCAGGGUCAGUUUUAGAGCUAGAAAUAGCAAGUUAAAAUAAGG | 17324 |
| | P10R11_del7- | CUAGUCCGUUAUCAACUUGAAAAAGUGGCACCGAGUCGGUGCAGAUAUCCAGACC | |
| | nick | CUGCCGUG | |
| RNACS6170 | TRAC5_ | GCUGGUACACGGCAGGGUCAGUUUUAGAGCUAGAAAUAGCAAGUUAAAAUAAGG | 17325 |
| | P9R11_del7- | CUAGUCCGUUAUCAACUUGAAAAAGUGGCACCGAGUCGGUGCAGAUAUCCAGACC | |
| | nick | CUGCCGU | |
| RNACS6171 | TRAC5_ | GCUGGUACACGGCAGGGUCAGUUUUAGAGCUAGAAAUAGCAAGUUAAAAUAAGG | 17326 |
| | P8R11_del7- | CUAGUCCGUUAUCAACUUGAAAAAGUGGCACCGAGUCGGUGCAGAUAUCCAGACC | |
| | nick | CUGCCG | |
| RNACS6172 | TRAC5_ | GCUGGUACACGGCAGGGUCAGUUUUAGAGCUAGAAAUAGCAAGUUAAAAUAAGG | 17327 |
| | P13R13_del7- | CUAGUCCGUUAUCAACUUGAAAAAGUGGCACCGAGUCGGUGCACAGAUAUCCAGA | |
| | nick | CCCUGCCGUGUAC | |
| RNACS6173 | TRAC5_ | GCUGGUACACGGCAGGGUCAGUUUUAGAGCUAGAAAUAGCAAGUUAAAAUAAGG | 17328 |
| | P12R13_del7- | CUAGUCCGUUAUCAACUUGAAAAAGUGGCACCGAGUCGGUGCACAGAUAUCCAGA | |
| | nick | CCCUGCCGUGUA | |
| RNACS6174 | TRAC5_ | GCUGGUACACGGCAGGGUCAGUUUUAGAGCUAGAAAUAGCAAGUUAAAAUAAGG | 17329 |
| | P11R13_del7- | CUAGUCCGUUAUCAACUUGAAAAAGUGGCACCGAGUCGGUGCACAGAUAUCCAGA | |
| | nick | CCCUGCCGUGU | |
| RNACS6175 | TRAC5_ | GCUGGUACACGGCAGGGUCAGUUUUAGAGCUAGAAAUAGCAAGUUAAAAUAAGG | 17330 |
| | P10R13_del7- | CUAGUCCGUUAUCAACUUGAAAAAGUGGCACCGAGUCGGUGCACAGAUAUCCAGA | |
| | nick | CCCUGCCGUG | |
| RNACS6176 | TRAC5_ | GCUGGUACACGGCAGGGUCAGUUUUAGAGCUAGAAAUAGCAAGUUAAAAUAAGG | 17331 |
| | P9R13_del7- | CUAGUCCGUUAUCAACUUGAAAAAGUGGCACCGAGUCGGUGCACAGAUAUCCAGA | |
| | nick | CCCUGCCGU | |
| RNACS6177 | TRAC5_ | GCUGGUACACGGCAGGGUCAGUUUUAGAGCUAGAAAUAGCAAGUUAAAAUAAGG | 17332 |
| | P8R13_del7- | CUAGUCCGUUAUCAACUUGAAAAAGUGGCACCGAGUCGGUGCACAGAUAUCCAGA | |
| | nick | CCCUGCCG | |
| RNACS6178 | TRAC5_ | GCUGGUACACGGCAGGGUCAGUUUUAGAGCUAGAAAUAGCAAGUUAAAAUAAGG | 17333 |
| | P13R15_del7- | CUAGUCCGUUAUCAACUUGAAAAAGUGGCACCGAGUCGGUGCCCACAGAUAUCCA | |
| | nick | GACCCUGCCGUGUAC | |
| RNACS6179 | TRAC5_ | CUAGUCCGUUAUCAACUUGAAAAAGUGGCACCGAGUCGGUGCCCACAGAUAUCCA | 17334 |
| | P12R15_del7- | GCUGGUACACGGCAGGGUCAGUUUUAGAGCUAGAAAUAGCAAGUUAAAAUAAGG | |
| | nick | GACCCUGCCGUGUA | |
| RNACS6180 | TRAC5_ | GCUGGUACACGGCAGGGUCAGUUUUAGAGCUAGAAAUAGCAAGUUAAAAUAAGG | 17335 |
| | P11R15_del7- | CUAGUCCGUUAUCAACUUGAAAAAGUGGCACCGAGUCGGUGCCCACAGAUAUCCA | |
| | nick | GACCCUGCCGUGU | |
| RNACS6181 | TRAC5_ | GCUGGUACACGGCAGGGUCAGUUUUAGAGCUAGAAAUAGCAAGUUAAAAUAAGG | 17336 |
| | P10R15_del7- | CUAGUCCGUUAUCAACUUGAAAAAGUGGCACCGAGUCGGUGCCCACAGAUAUCCA | |
| | nick | GACCCUGCCGUG | |
| RNACS6182 | TRAC5_ | GCUGGUACACGGCAGGGUCAGUUUUAGAGCUAGAAAUAGCAAGUUAAAAUAAGG | 17337 |
| | P9R15_del7- | CUAGUCCGUUAUCAACUUGAAAAAGUGGCACCGAGUCGGUGCCCACAGAUAUCCA | |
| | nick | GACCCUGCCGU | |
| RNACS6183 | TRAC5_ | GCUGGUACACGGCAGGGUCAGUUUUAGAGCUAGAAAUAGCAAGUUAAAAUAAGG | 17338 |
| | P8R15_del7- | CUAGUCCGUUAUCAACUUGAAAAAGUGGCACCGAGUCGGUGCCCACAGAUAUCCA | |
| | nick | GACCCUGCCG | |
| RNACS6184 | TRAC5_ | GCUGGUACACGGCAGGGUCAGUUUUAGAGCUAGAAAUAGCAAGUUAAAAUAAGG | 17339 |
| | P13R17_del7- | CUAGUCCGUUAUCAACUUGAAAAAGUGGCACCGAGUCGGUGCUCCCACAGAUAUC | |
| | nick | CAGACCCUGCCGUGUAC | |
| RNACS6185 | TRAC5_ | GCUGGUACACGGCAGGGUCAGUUUUAGAGCUAGAAAUAGCAAGUUAAAAUAAGG | 17340 |
| | P12R17_del7- | CUAGUCCGUUAUCAACUUGAAAAAGUGGCACCGAGUCGGUGCUCCCACAGAUAUC | |
| | nick | CAGACCCUGCCGUGUA | |
| RNACS6186 | TRAC5_ | GCUGGUACACGGCAGGGUCAGUUUUAGAGCUAGAAAUAGCAAGUUAAAAUAAGG | 17341 |
| | P11R17_del7- | CUAGUCCGUUAUCAACUUGAAAAAGUGGCACCGAGUCGGUGCUCCCACAGAUAUC | |
| | nick | CAGACCCUGCCGUGU | |
| RNACS6187 | TRAC5_ | GCUGGUACACGGCAGGGUCAGUUUUAGAGCUAGAAAUAGCAAGUUAAAAUAAGG | 17342 |
| | P10R17_del7- | CUAGUCCGUUAUCAACUUGAAAAAGUGGCACCGAGUCGGUGCUCCCACAGAUAUC | |
| | nick | CAGACCCUGCCGUG | |
| RNACS6188 | TRAC5_ | GCUGGUACACGGCAGGGUCAGUUUUAGAGCUAGAAAUAGCAAGUUAAAAUAAGG | 17343 |
| | P9R17_del7- | CUAGUCCGUUAUCAACUUGAAAAAGUGGCACCGAGUCGGUGCUCCCACAGAUAUC | |
| | nick | CAGACCCUGCCGU | |
| RNACS6189 | TRAC5_ | GCUGGUACACGGCAGGGUCAGUUUUAGAGCUAGAAAUAGCAAGUUAAAAUAAGG | 17344 |
| | P8R17_del7- | CUAGUCCGUUAUCAACUUGAAAAAGUGGCACCGAGUCGGUGCUCCCACAGAUAUC | |
| | nick | CAGACCCUGCCG | |
| RNACS6190 | TRAC5_ | GCUGGUACACGGCAGGGUCAGUUUUAGAGCUAGAAAUAGCAAGUUAAAAUAAGG | 17345 |
| | P13R13_ins5- | CUAGUCCGUUAUCAACUUGAAAAAGUGGCACCGAGUCGGUGCAACCCUAGUGUGA | |
| | PAM | CCCUGCCGUGUAC | |
| RNACS6191 | TRAC5_ | GCUGGUACACGGCAGGGUCAGUUUUAGAGCUAGAAAUAGCAAGUUAAAAUAAGG | 17346 |
| | P12R13_ins5- | CUAGUCCGUUAUCAACUUGAAAAAGUGGCACCGAGUCGGUGCAACCCUAGUGUGA | |
| | PAM | CCCUGCCGUGUA | |
| RNACS6192 | TRAC5_ | GCUGGUACACGGCAGGGUCAGUUUUAGAGCUAGAAAUAGCAAGUUAAAAUAAGG | 17347 |
| | P11R13_ins5- | CUAGUCCGUUAUCAACUUGAAAAAGUGGCACCGAGUCGGUGCAACCCUAGUGUGA | |
| | PAM | CCCUGCCGUGU | |
| RNACS6193 | TRAC5_ | GCUGGUACACGGCAGGGUCAGUUUUAGAGCUAGAAAUAGCAAGUUAAAAUAAGG | 17348 |
| | P10R13_ins5- | CUAGUCCGUUAUCAACUUGAAAAAGUGGCACCGAGUCGGUGCAACCCUAGUGUGA | |
| | PAM | CCCUGCCGUG | |
| RNACS6194 | TRAC5_ | GCUGGUACACGGCAGGGUCAGUUUUAGAGCUAGAAAUAGCAAGUUAAAAUAAGG | 17349 |
| | P9R13_ins5- | CUAGUCCGUUAUCAACUUGAAAAAGUGGCACCGAGUCGGUGCAACCCUAGUGUGA | |
| | PAM | CCCUGCCGU | |
| RNACS6195 | TRAC5_ | GCUGGUACACGGCAGGGUCAGUUUUAGAGCUAGAAAUAGCAAGUUAAAAUAAGG | 17350 |
| | P8R13_ins5- | CUAGUCCGUUAUCAACUUGAAAAAGUGGCACCGAGUCGGUGCAACCCUAGUGUGA | |
| | PAM | CCCUGCCG | |
| RNACS6196 | TRAC5_ | GCUGGUACACGGCAGGGUCAGUUUUAGAGCUAGAAAUAGCAAGUUAAAAUAAGG | 17351 |
| | P13R15_ins5- | CUAGUCCGUUAUCAACUUGAAAAAGUGGCACCGAGUCGGUGCAGAACCCUAGUGU | |
| | PAM | GACCCUGCCGUGUAC | |
| RNACS6197 | TRAC5_ | GCUGGUACACGGCAGGGUCAGUUUUAGAGCUAGAAAUAGCAAGUUAAAAUAAGG | 17352 |
| | P12R15_ins5- | CUAGUCCGUUAUCAACUUGAAAAAGUGGCACCGAGUCGGUGCAGAACCCUAGUGU | |
| | PAM | GACCCUGCCGUGUA | |
| RNACS6198 | TRAC5_ | GCUGGUACACGGCAGGGUCAGUUUUAGAGCUAGAAAUAGCAAGUUAAAAUAAGG | 17353 |
| | P11R15_ins5- | CUAGUCCGUUAUCAACUUGAAAAAGUGGCACCGAGUCGGUGCAGAACCCUAGUGU | |
| | PAM | GACCCUGCCGUGU | |
| RNACS6199 | TRAC5_ | GCUGGUACACGGCAGGGUCAGUUUUAGAGCUAGAAAUAGCAAGUUAAAAUAAGG | 17354 |
| | P10R15_ins5- | CUAGUCCGUUAUCAACUUGAAAAAGUGGCACCGAGUCGGUGCAGAACCCUAGUGU | |
| | PAM | GACCCUGCCGUG | |
| RNACS6200 | TRAC5_ | GCUGGUACACGGCAGGGUCAGUUUUAGAGCUAGAAAUAGCAAGUUAAAAUAAGG | 17355 |
| | P9R15_ins5- | CUAGUCCGUUAUCAACUUGAAAAAGUGGCACCGAGUCGGUGCAGAACCCUAGUGU | |
| | PAM | GACCCUGCCGU | |
| RNACS6201 | TRAC5_ | GCUGGUACACGGCAGGGUCAGUUUUAGAGCUAGAAAUAGCAAGUUAAAAUAAGG | 17356 |
| | P8R15_ins5- | CUAGUCCGUUAUCAACUUGAAAAAGUGGCACCGAGUCGGUGCAGAACCCUAGUGU | |
| | PAM | GACCCUGCCG | |
| RNACS6202 | TRAC5_ | GCUGGUACACGGCAGGGUCAGUUUUAGAGCUAGAAAUAGCAAGUUAAAAUAAGG | 17357 |
| | P13R17_ins5- | CUAGUCCGUUAUCAACUUGAAAAAGUGGCACCGAGUCGGUGCCCAGAACCCUAGU | |
| | PAM | GUGACCCUGCCGUGUAC | |
| RNACS6203 | TRAC5_ | GCUGGUACACGGCAGGGUCAGUUUUAGAGCUAGAAAUAGCAAGUUAAAAUAAGG | 17358 |
| | P12R17_ins5- | CUAGUCCGUUAUCAACUUGAAAAAGUGGCACCGAGUCGGUGCCCAGAACCCUAGU | |
| | PAM | GUGACCCUGCCGUGUA | |
| RNACS6204 | TRAC5_ | GCUGGUACACGGCAGGGUCAGUUUUAGAGCUAGAAAUAGCAAGUUAAAAUAAGG | 17359 |
| | P11R17_ins5- | CUAGUCCGUUAUCAACUUGAAAAAGUGGCACCGAGUCGGUGCCCAGAACCCUAGU | |
| | PAM | GUGACCCUGCCGUGU | |
| RNACS6205 | TRAC5_ | GCUGGUACACGGCAGGGUCAGUUUUAGAGCUAGAAAUAGCAAGUUAAAAUAAGG | 17360 |
| | P10R17_ins5- | CUAGUCCGUUAUCAACUUGAAAAAGUGGCACCGAGUCGGUGCCCAGAACCCUAGU | |
| | PAM | GUGACCCUGCCGUG | |
| RNACS6206 | TRAC5_ | GCUGGUACACGGCAGGGUCAGUUUUAGAGCUAGAAAUAGCAAGUUAAAAUAAGG | 17361 |
| | P9R17_ins5- | CUAGUCCGUUAUCAACUUGAAAAAGUGGCACCGAGUCGGUGCCCAGAACCCUAGU | |
| | PAM | GUGACCCUGCCGU | |
| RNACS6207 | TRAC5_ | GCUGGUACACGGCAGGGUCAGUUUUAGAGCUAGAAAUAGCAAGUUAAAAUAAGG | 17362 |
| | P8R17_ins5- | CUAGUCCGUUAUCAACUUGAAAAAGUGGCACCGAGUCGGUGCCCAGAACCCUAGU | |
| | PAM | GUGACCCUGCCG | |
| RNACS6208 | TRAC5_ | GCUGGUACACGGCAGGGUCAGUUUUAGAGCUAGAAAUAGCAAGUUAAAAUAAGG | 17363 |
| | P13R19_ins5- | CUAGUCCGUUAUCAACUUGAAAAAGUGGCACCGAGUCGGUGCAUCCAGAACCCUA | |
| | PAM | GUGUGACCCUGCCGUGUAC | |
| RNACS6209 | TRAC5_ | GCUGGUACACGGCAGGGUCAGUUUUAGAGCUAGAAAUAGCAAGUUAAAAUAAGG | 17364 |
| | P12R19_ins5- | CUAGUCCGUUAUCAACUUGAAAAAGUGGCACCGAGUCGGUGCAUCCAGAACCCUA | |
| | PAM | GUGUGACCCUGCCGUGUA | |
| RNACS6210 | TRAC5_ | GCUGGUACACGGCAGGGUCAGUUUUAGAGCUAGAAAUAGCAAGUUAAAAUAAGG | 17365 |
| | P11R19_ins5- | CUAGUCCGUUAUCAACUUGAAAAAGUGGCACCGAGUCGGUGCAUCCAGAACCCUA | |
| | PAM | GUGUGACCCUGCCGUGU | |
| RNACS6211 | TRAC5_ | GCUGGUACACGGCAGGGUCAGUUUUAGAGCUAGAAAUAGCAAGUUAAAAUAAGG | 17366 |
| | P10R19_ins5- | CUAGUCCGUUAUCAACUUGAAAAAGUGGCACCGAGUCGGUGCAUCCAGAACCCUA | |
| | PAM | GUGUGACCCUGCCGUG | |
| RNACS6212 | TRAC5_ | GCUGGUACACGGCAGGGUCAGUUUUAGAGCUAGAAAUAGCAAGUUAAAAUAAGG | 17367 |
| | P9R19_ins5- | CUAGUCCGUUAUCAACUUGAAAAAGUGGCACCGAGUCGGUGCAUCCAGAACCCUA | |
| | PAM | GUGUGACCCUGCCGU | |
| RNACS6213 | TRAC5_ | GCUGGUACACGGCAGGGUCAGUUUUAGAGCUAGAAAUAGCAAGUUAAAAUAAGG | 17368 |
| | P8R19_ins5- | CUAGUCCGUUAUCAACUUGAAAAAGUGGCACCGAGUCGGUGCAUCCAGAACCCUA | |
| | PAM | GUGUGACCCUGCCG | |
| RNACS6214 | TRAC5_ | GCUGGUACACGGCAGGGUCAGUUUUAGAGCUAGAAAUAGCAAGUUAAAAUAAGG | 17369 |
| | P13R21_ins5- | CUAGUCCGUUAUCAACUUGAAAAAGUGGCACCGAGUCGGUGCAUAUCCAGAACCC | |
| | PAM | UAGUGUGACCCUGCCGUGUAC | |
| RNACS6215 | TRAC5_ | GCUGGUACACGGCAGGGUCAGUUUUAGAGCUAGAAAUAGCAAGUUAAAAUAAGG | 17370 |
| | P12R21_ins5- | CUAGUCCGUUAUCAACUUGAAAAAGUGGCACCGAGUCGGUGCAUAUCCAGAACCC | |
| | PAM | UAGUGUGACCCUGCCGUGUA | |
| RNACS6216 | TRAC5_ | GCUGGUACACGGCAGGGUCAGUUUUAGAGCUAGAAAUAGCAAGUUAAAAUAAGG | 17371 |
| | P11R21_ins5- | CUAGUCCGUUAUCAACUUGAAAAAGUGGCACCGAGUCGGUGCAUAUCCAGAACCC | |
| | PAM | UAGUGUGACCCUGCCGUGU | |
| RNACS6217 | TRAC5_ | GCUGGUACACGGCAGGGUCAGUUUUAGAGCUAGAAAUAGCAAGUUAAAAUAAGG | 17372 |
| | P10R21_ins5- | CUAGUCCGUUAUCAACUUGAAAAAGUGGCACCGAGUCGGUGCAUAUCCAGAACCC | |
| | PAM | UAGUGUGACCCUGCCGUG | |
| RNACS6218 | TRAC5_ | GCUGGUACACGGCAGGGUCAGUUUUAGAGCUAGAAAUAGCAAGUUAAAAUAAGG | 17373 |
| | P9R21_ins5- | CUAGUCCGUUAUCAACUUGAAAAAGUGGCACCGAGUCGGUGCAUAUCCAGAACCC | |
| | PAM | UAGUGUGACCCUGCCGU | |
| RNACS6219 | TRAC5_ | GCUGGUACACGGCAGGGUCAGUUUUAGAGCUAGAAAUAGCAAGUUAAAAUAAGG | 17374 |
| | P8R21_ins5- | CUAGUCCGUUAUCAACUUGAAAAAGUGGCACCGAGUCGGUGCAUAUCCAGAACCC | |
| | PAM | UAGUGUGACCCUGCCG | |
| RNACS6220 | TRAC5_ | GCUGGUACACGGCAGGGUCAGUUUUAGAGCUAGAAAUAGCAAGUUAAAAUAAGG | 17375 |
| | P13R23_ins5- | CUAGUCCGUUAUCAACUUGAAAAAGUGGCACCGAGUCGGUGCAGAUAUCCAGAAC | |
| | PAM | CCUAGUGUGACCCUGCCGUGUAC | |
| RNACS6221 | TRAC5_ | GCUGGUACACGGCAGGGUCAGUUUUAGAGCUAGAAAUAGCAAGUUAAAAUAAGG | 17376 |
| | P12R23_ins5- | CUAGUCCGUUAUCAACUUGAAAAAGUGGCACCGAGUCGGUGCAGAUAUCCAGAAC | |
| | PAM | CCUAGUGUGACCCUGCCGUGUA | |
| RNACS6222 | TRAC5_ | GCUGGUACACGGCAGGGUCAGUUUUAGAGCUAGAAAUAGCAAGUUAAAAUAAGG | 17377 |
| | P11R23_ins5- | CUAGUCCGUUAUCAACUUGAAAAAGUGGCACCGAGUCGGUGCAGAUAUCCAGAAC | |
| | PAM | CCUAGUGUGACCCUGCCGUGU | |
| RNACS6223 | TRAC5_ | GCUGGUACACGGCAGGGUCAGUUUUAGAGCUAGAAAUAGCAAGUUAAAAUAAGG | 17378 |
| | P10R23_ins5- | CUAGUCCGUUAUCAACUUGAAAAAGUGGCACCGAGUCGGUGCAGAUAUCCAGAAC | |
| | PAM | CCUAGUGUGACCCUGCCGUG | |
| RNACS6224 | TRAC5_ | GCUGGUACACGGCAGGGUCAGUUUUAGAGCUAGAAAUAGCAAGUUAAAAUAAGG | 17379 |
| | P9R23_ins5- | CUAGUCCGUUAUCAACUUGAAAAAGUGGCACCGAGUCGGUGCAGAUAUCCAGAAC | |
| | PAM | CCUAGUGUGACCCUGCCGU | |
| RNACS6225 | TRAC5_ | GCUGGUACACGGCAGGGUCAGUUUUAGAGCUAGAAAUAGCAAGUUAAAAUAAGG | 17380 |
| | P8R23_ins5- | CUAGUCCGUUAUCAACUUGAAAAAGUGGCACCGAGUCGGUGCAGAUAUCCAGAAC | |
| | PAM | CCUAGUGUGACCCUGCCG | |
| RNACS6226 | TRAC5_ | GCUGGUACACGGCAGGGUCAGUUUUAGAGCUAGAAAUAGCAAGUUAAAAUAAGG | 17381 |
| | P13R25_ins5- | CUAGUCCGUUAUCAACUUGAAAAAGUGGCACCGAGUCGGUGCACAGAUAUCCAGA | |
| | PAM | ACCCUAGUGUGACCCUGCCGUGUAC | |
| RNACS6227 | TRAC5_ | GCUGGUACACGGCAGGGUCAGUUUUAGAGCUAGAAAUAGCAAGUUAAAAUAAGG | 17382 |
| | P12R25_ins5- | CUAGUCCGUUAUCAACUUGAAAAAGUGGCACCGAGUCGGUGCACAGAUAUCCAGA | |
| | PAM | ACCCUAGUGUGACCCUGCCGUGUA | |
| RNACS6228 | TRAC5_ | GCUGGUACACGGCAGGGUCAGUUUUAGAGCUAGAAAUAGCAAGUUAAAAUAAGG | 17383 |
| | P11R25_ins5- | CUAGUCCGUUAUCAACUUGAAAAAGUGGCACCGAGUCGGUGCACAGAUAUCCAGA | |
| | PAM | ACCCUAGUGUGACCCUGCCGUGU | |
| RNACS6229 | TRAC5_ | GCUGGUACACGGCAGGGUCAGUUUUAGAGCUAGAAAUAGCAAGUUAAAAUAAGG | 17384 |
| | P10R25_ins5- | CUAGUCCGUUAUCAACUUGAAAAAGUGGCACCGAGUCGGUGCACAGAUAUCCAGA | |
| | PAM | ACCCUAGUGUGACCCUGCCGUG | |
| RNACS6230 | TRAC5_ | GCUGGUACACGGCAGGGUCAGUUUUAGAGCUAGAAAUAGCAAGUUAAAAUAAGG | 17385 |
| | P9R25_ins5- | CUAGUCCGUUAUCAACUUGAAAAAGUGGCACCGAGUCGGUGCACAGAUAUCCAGA | |
| | PAM | ACCCUAGUGUGACCCUGCCGU | |
| RNACS6231 | TRAC5_ | GCUGGUACACGGCAGGGUCAGUUUUAGAGCUAGAAAUAGCAAGUUAAAAUAAGG | 17386 |
| | P8R25_ins5- | CUAGUCCGUUAUCAACUUGAAAAAGUGGCACCGAGUCGGUGCACAGAUAUCCAGA | |
| | PAM | ACCCUAGUGUGACCCUGCCG | |
| RNACS6232 | TRAC5_ | GCUGGUACACGGCAGGGUCAGUUUUAGAGCUAGAAAUAGCAAGUUAAAAUAAGG | 17387 |
| | P13R15_ins7- | CUAGUCCGUUAUCAACUUGAAAAAGUGGCACCGAGUCGGUGCAACCCUAGUUGAU | |
| | PAM | GACCCUGCCGUGUAC | |
| RNACS6233 | TRAC5_ | GCUGGUACACGGCAGGGUCAGUUUUAGAGCUAGAAAUAGCAAGUUAAAAUAAGG | 17388 |
| | P12R15_ins7- | CUAGUCCGUUAUCAACUUGAAAAAGUGGCACCGAGUCGGUGCAACCCUAGUUGAU | |
| | PAM | GACCCUGCCGUGUA | |
| RNACS6234 | TRAC5_ | GCUGGUACACGGCAGGGUCAGUUUUAGAGCUAGAAAUAGCAAGUUAAAAUAAGG | 17389 |
| | P11R15_ins7- | CUAGUCCGUUAUCAACUUGAAAAAGUGGCACCGAGUCGGUGCAACCCUAGUUGAU | |
| | PAM | GACCCUGCCGUGU | |
| RNACS6235 | TRAC5_ | GCUGGUACACGGCAGGGUCAGUUUUAGAGCUAGAAAUAGCAAGUUAAAAUAAGG | 17390 |
| | P10R15_ins7- | CUAGUCCGUUAUCAACUUGAAAAAGUGGCACCGAGUCGGUGCAACCCUAGUUGAU | |
| | PAM | GACCCUGCCGUG | |
| RNACS6236 | TRAC5_ | GCUGGUACACGGCAGGGUCAGUUUUAGAGCUAGAAAUAGCAAGUUAAAAUAAGG | 17391 |
| | P9R15_ins7- | CUAGUCCGUUAUCAACUUGAAAAAGUGGCACCGAGUCGGUGCAACCCUAGUUGAU | |
| | PAM | GACCCUGCCGU | |
| RNACS6237 | TRAC5_ | GCUGGUACACGGCAGGGUCAGUUUUAGAGCUAGAAAUAGCAAGUUAAAAUAAGG | 17392 |
| | P8R15_ins7- | CUAGUCCGUUAUCAACUUGAAAAAGUGGCACCGAGUCGGUGCAACCCUAGUUGAU | |
| | PAM | GACCCUGCCG | |
| RNACS6238 | TRAC5_ | GCUGGUACACGGCAGGGUCAGUUUUAGAGCUAGAAAUAGCAAGUUAAAAUAAGG | 17393 |
| | P13R17_ins7- | CUAGUCCGUUAUCAACUUGAAAAAGUGGCACCGAGUCGGUGCAGAACCCUAGUUG | |
| | PAM | AUGACCCUGCCGUGUAC | |
| RNACS6239 | TRAC5_ | GCUGGUACACGGCAGGGUCAGUUUUAGAGCUAGAAAUAGCAAGUUAAAAUAAGG | 17394 |
| | P12R17_ins7- | CUAGUCCGUUAUCAACUUGAAAAAGUGGCACCGAGUCGGUGCAGAACCCUAGUUG | |
| | PAM | AUGACCCUGCCGUGUA | |
| RNACS6240 | TRAC5_ | GCUGGUACACGGCAGGGUCAGUUUUAGAGCUAGAAAUAGCAAGUUAAAAUAAGG | 17395 |
| | P11R17_ins7- | CUAGUCCGUUAUCAACUUGAAAAAGUGGCACCGAGUCGGUGCAGAACCCUAGUUG | |
| | PAM | AUGACCCUGCCGUGU | |
| RNACS6241 | TRAC5_ | GCUGGUACACGGCAGGGUCAGUUUUAGAGCUAGAAAUAGCAAGUUAAAAUAAGG | 17396 |
| | P10R17_ins7- | CUAGUCCGUUAUCAACUUGAAAAAGUGGCACCGAGUCGGUGCAGAACCCUAGUUG | |
| | PAM | AUGACCCUGCCGUG | |
| RNACS6242 | TRAC5_ | GCUGGUACACGGCAGGGUCAGUUUUAGAGCUAGAAAUAGCAAGUUAAAAUAAGG | 17397 |
| | P9R17_ins7- | CUAGUCCGUUAUCAACUUGAAAAAGUGGCACCGAGUCGGUGCAGAACCCUAGUUG | |
| | PAM | AUGACCCUGCCGU | |
| RNACS6243 | TRAC5_ | GCUGGUACACGGCAGGGUCAGUUUUAGAGCUAGAAAUAGCAAGUUAAAAUAAGG | 17398 |
| | P8R17_ins7- | CUAGUCCGUUAUCAACUUGAAAAAGUGGCACCGAGUCGGUGCAGAACCCUAGUUG | |
| | PAM | AUGACCCUGCCG | |
| RNACS6244 | TRAC5_ | GCUGGUACACGGCAGGGUCAGUUUUAGAGCUAGAAAUAGCAAGUUAAAAUAAGG | 17399 |
| | P13R19_ins7- | CUAGUCCGUUAUCAACUUGAAAAAGUGGCACCGAGUCGGUGCCCAGAACCCUAGU | |
| | PAM | UGAUGACCCUGCCGUGUAC | |
| RNACS6245 | TRAC5_ | GCUGGUACACGGCAGGGUCAGUUUUAGAGCUAGAAAUAGCAAGUUAAAAUAAGG | 17400 |
| | P12R19_ins7- | CUAGUCCGUUAUCAACUUGAAAAAGUGGCACCGAGUCGGUGCCCAGAACCCUAGU | |
| | PAM | UGAUGACCCUGCCGUGUA | |
| RNACS6246 | TRAC5_ | GCUGGUACACGGCAGGGUCAGUUUUAGAGCUAGAAAUAGCAAGUUAAAAUAAGG | 17401 |
| | P11R19_ins7- | CUAGUCCGUUAUCAACUUGAAAAAGUGGCACCGAGUCGGUGCCCAGAACCCUAGU | |
| | PAM | UGAUGACCCUGCCGUGU | |
| RNACS6247 | TRAC5_ | GCUGGUACACGGCAGGGUCAGUUUUAGAGCUAGAAAUAGCAAGUUAAAAUAAGG | 17402 |
| | P10R19_ins7- | CUAGUCCGUUAUCAACUUGAAAAAGUGGCACCGAGUCGGUGCCCAGAACCCUAGU | |
| | PAM | UGAUGACCCUGCCGUG | |
| RNACS6248 | TRAC5_ | GCUGGUACACGGCAGGGUCAGUUUUAGAGCUAGAAAUAGCAAGUUAAAAUAAGG | 17403 |
| | P9R19_ins7- | CUAGUCCGUUAUCAACUUGAAAAAGUGGCACCGAGUCGGUGCCCAGAACCCUAGU | |
| | PAM | UGAUGACCCUGCCGU | |
| RNACS6249 | TRAC5_ | GCUGGUACACGGCAGGGUCAGUUUUAGAGCUAGAAAUAGCAAGUUAAAAUAAGG | 17404 |
| | P8R19_ins7- | CUAGUCCGUUAUCAACUUGAAAAAGUGGCACCGAGUCGGUGCCCAGAACCCUAGU | |
| | PAM | UGAUGACCCUGCCG | |
| RNACS6250 | TRAC5_ | GCUGGUACACGGCAGGGUCAGUUUUAGAGCUAGAAAUAGCAAGUUAAAAUAAGG | 17405 |
| | P13R21_ins7- | CUAGUCCGUUAUCAACUUGAAAAAGUGGCACCGAGUCGGUGCAUCCAGAACCCUA | |
| | PAM | GUUGAUGACCCUGCCGUGUAC | |
| RNACS6251 | TRAC5_ | GCUGGUACACGGCAGGGUCAGUUUUAGAGCUAGAAAUAGCAAGUUAAAAUAAGG | 17406 |
| | P12R21_ins7- | CUAGUCCGUUAUCAACUUGAAAAAGUGGCACCGAGUCGGUGCAUCCAGAACCCUA | |
| | PAM | GUUGAUGACCCUGCCGUGUA | |
| RNACS6252 | TRAC5_ | GCUGGUACACGGCAGGGUCAGUUUUAGAGCUAGAAAUAGCAAGUUAAAAUAAGG | 17407 |
| | P11R21_ins7- | CUAGUCCGUUAUCAACUUGAAAAAGUGGCACCGAGUCGGUGCAUCCAGAACCCUA | |
| | PAM | GUUGAUGACCCUGCCGUGU | |
| RNACS6253 | TRAC5_ | GCUGGUACACGGCAGGGUCAGUUUUAGAGCUAGAAAUAGCAAGUUAAAAUAAGG | 17408 |
| | P10R21_ins7- | CUAGUCCGUUAUCAACUUGAAAAAGUGGCACCGAGUCGGUGCAUCCAGAACCCUA | |
| | PAM | GUUGAUGACCCUGCCGUG | |
| RNACS6254 | TRAC5_ | GCUGGUACACGGCAGGGUCAGUUUUAGAGCUAGAAAUAGCAAGUUAAAAUAAGG | 17409 |
| | P9R21_ins7- | CUAGUCCGUUAUCAACUUGAAAAAGUGGCACCGAGUCGGUGCAUCCAGAACCCUA | |
| | PAM | GUUGAUGACCCUGCCGU | |
| RNACS6255 | TRAC5_ | GCUGGUACACGGCAGGGUCAGUUUUAGAGCUAGAAAUAGCAAGUUAAAAUAAGG | 17410 |
| | P8R21_ins7- | CUAGUCCGUUAUCAACUUGAAAAAGUGGCACCGAGUCGGUGCAUCCAGAACCCUA | |
| | PAM | GUUGAUGACCCUGCCG | |
| RNACS6256 | TRAC5_ | GCUGGUACACGGCAGGGUCAGUUUUAGAGCUAGAAAUAGCAAGUUAAAAUAAGG | 17411 |
| | P13R23_ins7- | CUAGUCCGUUAUCAACUUGAAAAAGUGGCACCGAGUCGGUGCAUAUCCAGAACCC | |
| | PAM | UAGUUGAUGACCCUGCCGUGUAC | |
| RNACS6257 | TRAC5_ | GCUGGUACACGGCAGGGUCAGUUUUAGAGCUAGAAAUAGCAAGUUAAAAUAAGG | 17412 |
| | P12R23_ins7- | CUAGUCCGUUAUCAACUUGAAAAAGUGGCACCGAGUCGGUGCAUAUCCAGAACCC | |
| | PAM | UAGUUGAUGACCCUGCCGUGUA | |
| RNACS6258 | TRAC5_ | GCUGGUACACGGCAGGGUCAGUUUUAGAGCUAGAAAUAGCAAGUUAAAAUAAGG | 17413 |
| | P11R23_ins7- | CUAGUCCGUUAUCAACUUGAAAAAGUGGCACCGAGUCGGUGCAUAUCCAGAACCC | |
| | PAM | UAGUUGAUGACCCUGCCGUGU | |
| RNACS6259 | TRAC5_ | GCUGGUACACGGCAGGGUCAGUUUUAGAGCUAGAAAUAGCAAGUUAAAAUAAGG | 17414 |
| | P10R23_ins7- | CUAGUCCGUUAUCAACUUGAAAAAGUGGCACCGAGUCGGUGCAUAUCCAGAACCC | |
| | PAM | UAGUUGAUGACCCUGCCGUG | |
| RNACS6260 | TRAC5_ | GCUGGUACACGGCAGGGUCAGUUUUAGAGCUAGAAAUAGCAAGUUAAAAUAAGG | 17415 |
| | P9R23_ins7- | CUAGUCCGUUAUCAACUUGAAAAAGUGGCACCGAGUCGGUGCAUAUCCAGAACCC | |
| | PAM | UAGUUGAUGACCCUGCCGU | |
| RNACS6261 | TRAC5_ | GCUGGUACACGGCAGGGUCAGUUUUAGAGCUAGAAAUAGCAAGUUAAAAUAAGG | 17416 |
| | P8R23_ins7- | CUAGUCCGUUAUCAACUUGAAAAAGUGGCACCGAGUCGGUGCAUAUCCAGAACCC | |
| | PAM | UAGUUGAUGACCCUGCCG | |
| RNACS6262 | TRAC5_ | GCUGGUACACGGCAGGGUCAGUUUUAGAGCUAGAAAUAGCAAGUUAAAAUAAGG | 17417 |
| | P13R25_ins7- | CUAGUCCGUUAUCAACUUGAAAAAGUGGCACCGAGUCGGUGCAGAUAUCCAGAAC | |
| | PAM | CCUAGUUGAUGACCCUGCCGUGUAC | |
| RNACS6263 | TRAC5_ | GCUGGUACACGGCAGGGUCAGUUUUAGAGCUAGAAAUAGCAAGUUAAAAUAAGG | 17418 |
| | P12R25_ins7- | CUAGUCCGUUAUCAACUUGAAAAAGUGGCACCGAGUCGGUGCAGAUAUCCAGAAC | |
| | PAM | CCUAGUUGAUGACCCUGCCGUGUA | |
| RNACS6264 | TRAC5_ | GCUGGUACACGGCAGGGUCAGUUUUAGAGCUAGAAAUAGCAAGUUAAAAUAAGG | 17419 |
| | P11R25_ins7- | CUAGUCCGUUAUCAACUUGAAAAAGUGGCACCGAGUCGGUGCAGAUAUCCAGAAC | |
| | PAM | CCUAGUUGAUGACCCUGCCGUGU | |
| RNACS6265 | TRAC5_ | GCUGGUACACGGCAGGGUCAGUUUUAGAGCUAGAAAUAGCAAGUUAAAAUAAGG | 17420 |
| | P10R25_ins7- | CUAGUCCGUUAUCAACUUGAAAAAGUGGCACCGAGUCGGUGCAGAUAUCCAGAAC | |
| | PAM | CCUAGUUGAUGACCCUGCCGUG | |
| RNACS6266 | TRAC5_ | GCUGGUACACGGCAGGGUCAGUUUUAGAGCUAGAAAUAGCAAGUUAAAAUAAGG | 17421 |
| | P9R25_ins7- | CUAGUCCGUUAUCAACUUGAAAAAGUGGCACCGAGUCGGUGCAGAUAUCCAGAAC | |
| | PAM | CCUAGUUGAUGACCCUGCCGU | |
| RNACS6267 | TRAC5_ | GCUGGUACACGGCAGGGUCAGUUUUAGAGCUAGAAAUAGCAAGUUAAAAUAAGG | 17422 |
| | P8R25_ins7- | CUAGUCCGUUAUCAACUUGAAAAAGUGGCACCGAGUCGGUGCAGAUAUCCAGAAC | |
| | PAM | CCUAGUUGAUGACCCUGCCG | |
| RNACS6268 | TRAC5_ | GCUGGUACACGGCAGGGUCAGUUUUAGAGCUAGAAAUAGCAAGUUAAAAUAAGG | 17423 |
| | P13R27_ins7- | CUAGUCCGUUAUCAACUUGAAAAAGUGGCACCGAGUCGGUGCACAGAUAUCCAGA | |
| | PAM | ACCCUAGUUGAUGACCCUGCCGUGUAC | |
| RNACS6269 | TRAC5_ | GCUGGUACACGGCAGGGUCAGUUUUAGAGCUAGAAAUAGCAAGUUAAAAUAAGG | 17424 |
| | P12R27_ins7- | CUAGUCCGUUAUCAACUUGAAAAAGUGGCACCGAGUCGGUGCACAGAUAUCCAGA | |
| | PAM | ACCCUAGUUGAUGACCCUGCCGUGUA | |
| RNACS6270 | TRAC5_ | GCUGGUACACGGCAGGGUCAGUUUUAGAGCUAGAAAUAGCAAGUUAAAAUAAGG | 17425 |
| | P11R27_ins7- | CUAGUCCGUUAUCAACUUGAAAAAGUGGCACCGAGUCGGUGCACAGAUAUCCAGA | |
| | PAM | ACCCUAGUUGAUGACCCUGCCGUGU | |
| RNACS6271 | TRAC5_ | GCUGGUACACGGCAGGGUCAGUUUUAGAGCUAGAAAUAGCAAGUUAAAAUAAGG | 17426 |
| | P10R27_ins7- | CUAGUCCGUUAUCAACUUGAAAAAGUGGCACCGAGUCGGUGCACAGAUAUCCAGA | |
| | PAM | ACCCUAGUUGAUGACCCUGCCGUG | |
| RNACS6272 | TRAC5_ | GCUGGUACACGGCAGGGUCAGUUUUAGAGCUAGAAAUAGCAAGUUAAAAUAAGG | 17427 |
| | P9R27_ins7- | CUAGUCCGUUAUCAACUUGAAAAAGUGGCACCGAGUCGGUGCACAGAUAUCCAGA | |
| | PAM | ACCCUAGUUGAUGACCCUGCCGU | |
| RNACS6273 | TRAC5_ | GCUGGUACACGGCAGGGUCAGUUUUAGAGCUAGAAAUAGCAAGUUAAAAUAAGG | 17428 |
| | P8R27_ins7- | CUAGUCCGUUAUCAACUUGAAAAAGUGGCACCGAGUCGGUGCACAGAUAUCCAGA | |
| | PAM | ACCCUAGUUGAUGACCCUGCCG | |
| RNACS6274 | TRAC5_ | GCUGGUACACGGCAGGGUCAGUUUUAGAGCUAGAAAUAGCAAGUUAAAAUAAGG | 17429 |
| | P13R8_del5- | CUAGUCCGUUAUCAACUUGAAAAAGUGGCACCGAGUCGGUGCUCCAGUGACCCUG | |
| | PAM | CCGUGUAC | |
| RNACS6275 | TRAC5_ | GCUGGUACACGGCAGGGUCAGUUUUAGAGCUAGAAAUAGCAAGUUAAAAUAAGG | 17430 |
| | P12R8_del5- | CUAGUCCGUUAUCAACUUGAAAAAGUGGCACCGAGUCGGUGCUCCAGUGACCCUG | |
| | PAM | CCGUGUA | |
| RNACS6276 | TRAC5_ | GCUGGUACACGGCAGGGUCAGUUUUAGAGCUAGAAAUAGCAAGUUAAAAUAAGG | 17431 |
| | P11R8_del5- | CUAGUCCGUUAUCAACUUGAAAAAGUGGCACCGAGUCGGUGCUCCAGUGACCCUG | |
| | PAM | CCGUGU | |
| RNACS6277 | TRAC5_ | GCUGGUACACGGCAGGGUCAGUUUUAGAGCUAGAAAUAGCAAGUUAAAAUAAGG | 17432 |
| | P10R8 de15- | CUAGUCCGUUAUCAACUUGAAAAAGUGGCACCGAGUCGGUGCUCCAGUGACCCUG | |
| | PAM | CCGUG | |
| RNACS6278 | TRAC5_ | GCUGGUACACGGCAGGGUCAGUUUUAGAGCUAGAAAUAGCAAGUUAAAAUAAGG | 17433 |
| | P9R8_del5- | CUAGUCCGUUAUCAACUUGAAAAAGUGGCACCGAGUCGGUGCUCCAGUGACCCUG | |
| | PAM | CCGU | |
| RNACS6279 | TRAC5_ | GCUGGUACACGGCAGGGUCAGUUUUAGAGCUAGAAAUAGCAAGUUAAAAUAAGG | 17434 |
| | P8R8_del5- | CUAGUCCGUUAUCAACUUGAAAAAGUGGCACCGAGUCGGUGCUCCAGUGACCCUG | |
| | PAM | CCG | |
| RNACS6280 | TRAC5_ | GCUGGUACACGGCAGGGUCAGUUUUAGAGCUAGAAAUAGCAAGUUAAAAUAAGG | 17435 |
| | P13R10_del5- | CUAGUCCGUUAUCAACUUGAAAAAGUGGCACCGAGUCGGUGCUAUCCAGUGACCC | |
| | PAM | UGCCGUGUAC | |
| RNACS6281 | TRAC5_ | GCUGGUACACGGCAGGGUCAGUUUUAGAGCUAGAAAUAGCAAGUUAAAAUAAGG | 17436 |
| | P12R10_del5- | CUAGUCCGUUAUCAACUUGAAAAAGUGGCACCGAGUCGGUGCUAUCCAGUGACCC | |
| | PAM | UGCCGUGUA | |
| RNACS6282 | TRAC5_ | GCUGGUACACGGCAGGGUCAGUUUUAGAGCUAGAAAUAGCAAGUUAAAAUAAGG | 17437 |
| | P11R10_del5- | CUAGUCCGUUAUCAACUUGAAAAAGUGGCACCGAGUCGGUGCUAUCCAGUGACCC | |
| | PAM | UGCCGUGU | |
| RNACS6283 | TRAC5_ | GCUGGUACACGGCAGGGUCAGUUUUAGAGCUAGAAAUAGCAAGUUAAAAUAAGG | 17438 |
| | P10R10_del5- | CUAGUCCGUUAUCAACUUGAAAAAGUGGCACCGAGUCGGUGCUAUCCAGUGACCC | |
| | PAM | UGCCGUG | |
| RNACS6284 | TRAC5_ | GCUGGUACACGGCAGGGUCAGUUUUAGAGCUAGAAAUAGCAAGUUAAAAUAAGG | 17439 |
| | P9R10_del5- | CUAGUCCGUUAUCAACUUGAAAAAGUGGCACCGAGUCGGUGCUAUCCAGUGACCC | |
| | PAM | UGCCGU | |
| RNACS6285 | TRAC5_ | GCUGGUACACGGCAGGGUCAGUUUUAGAGCUAGAAAUAGCAAGUUAAAAUAAGG | 17440 |
| | P8R10 de15- | CUAGUCCGUUAUCAACUUGAAAAAGUGGCACCGAGUCGGUGCUAUCCAGUGACCC | |
| | PAM | UGCCG | |
| RNACS6286 | TRAC5_ | GCUGGUACACGGCAGGGUCAGUUUUAGAGCUAGAAAUAGCAAGUUAAAAUAAGG | 17441 |
| | P13R12_del5- | CUAGUCCGUUAUCAACUUGAAAAAGUGGCACCGAGUCGGUGCGAUAUCCAGUGAC | |
| | PAM | CCUGCCGUGUAC | |
| RNACS6287 | TRAC5_ | GCUGGUACACGGCAGGGUCAGUUUUAGAGCUAGAAAUAGCAAGUUAAAAUAAGG | 17442 |
| | P12R12_del5- | CUAGUCCGUUAUCAACUUGAAAAAGUGGCACCGAGUCGGUGCGAUAUCCAGUGAC | |
| | PAM | CCUGCCGUGUA | |
| RNACS6288 | TRAC5_ | GCUGGUACACGGCAGGGUCAGUUUUAGAGCUAGAAAUAGCAAGUUAAAAUAAGG | 17443 |
| | P11R12_del5- | CUAGUCCGUUAUCAACUUGAAAAAGUGGCACCGAGUCGGUGCGAUAUCCAGUGAC | |
| | PAM | CCUGCCGUGU | |
| RNACS6289 | TRAC5_ | GCUGGUACACGGCAGGGUCAGUUUUAGAGCUAGAAAUAGCAAGUUAAAAUAAGG | 17444 |
| | P10R12_del5- | CUAGUCCGUUAUCAACUUGAAAAAGUGGCACCGAGUCGGUGCGAUAUCCAGUGAC | |
| | PAM | CCUGCCGUG | |
| RNACS6290 | TRAC5_ | GCUGGUACACGGCAGGGUCAGUUUUAGAGCUAGAAAUAGCAAGUUAAAAUAAGG | 17445 |
| | P9R12_del5- | CUAGUCCGUUAUCAACUUGAAAAAGUGGCACCGAGUCGGUGCGAUAUCCAGUGAC | |
| | PAM | CCUGCCGU | |
| RNACS6291 | TRAC5_ | GCUGGUACACGGCAGGGUCAGUUUUAGAGCUAGAAAUAGCAAGUUAAAAUAAGG | 17446 |
| | P8R12_del5- | CUAGUCCGUUAUCAACUUGAAAAAGUGGCACCGAGUCGGUGCGAUAUCCAGUGAC | |
| | PAM | CCUGCCG | |
| RNACS6292 | TRAC5_ | GCUGGUACACGGCAGGGUCAGUUUUAGAGCUAGAAAUAGCAAGUUAAAAUAAGG | 17447 |
| | P13R14_del5- | CUAGUCCGUUAUCAACUUGAAAAAGUGGCACCGAGUCGGUGCCAGAUAUCCAGUG | |
| | PAM | ACCCUGCCGUGUAC | |
| RNACS6293 | TRAC5_ | GCUGGUACACGGCAGGGUCAGUUUUAGAGCUAGAAAUAGCAAGUUAAAAUAAGG | 17448 |
| | P12R14_del5- | CUAGUCCGUUAUCAACUUGAAAAAGUGGCACCGAGUCGGUGCCAGAUAUCCAGUG | |
| | PAM | ACCCUGCCGUGUA | |
| RNACS6294 | TRAC5_ | GCUGGUACACGGCAGGGUCAGUUUUAGAGCUAGAAAUAGCAAGUUAAAAUAAGG | 17449 |
| | P11R14_del5- | CUAGUCCGUUAUCAACUUGAAAAAGUGGCACCGAGUCGGUGCCAGAUAUCCAGUG | |
| | PAM | ACCCUGCCGUGU | |
| RNACS6295 | TRAC5_ | GCUGGUACACGGCAGGGUCAGUUUUAGAGCUAGAAAUAGCAAGUUAAAAUAAGG | 17450 |
| | P10R14_del5- | CUAGUCCGUUAUCAACUUGAAAAAGUGGCACCGAGUCGGUGCCAGAUAUCCAGUG | |
| | PAM | ACCCUGCCGUG | |
| RNACS6296 | TRAC5_ | GCUGGUACACGGCAGGGUCAGUUUUAGAGCUAGAAAUAGCAAGUUAAAAUAAGG | 17451 |
| | P9R14_del5- | CUAGUCCGUUAUCAACUUGAAAAAGUGGCACCGAGUCGGUGCCAGAUAUCCAGUG | |
| | PAM | ACCCUGCCGU | |
| RNACS6297 | TRAC5_ | GCUGGUACACGGCAGGGUCAGUUUUAGAGCUAGAAAUAGCAAGUUAAAAUAAGG | 17452 |
| | P8R14_del5- | CUAGUCCGUUAUCAACUUGAAAAAGUGGCACCGAGUCGGUGCCAGAUAUCCAGUG | |
| | PAM | ACCCUGCCG | |
| RNACS6298 | TRAC5_ | GCUGGUACACGGCAGGGUCAGUUUUAGAGCUAGAAAUAGCAAGUUAAAAUAAGG | 17453 |
| | P13R16_del5- | CUAGUCCGUUAUCAACUUGAAAAAGUGGCACCGAGUCGGUGCCACAGAUAUCCAG | |
| | PAM | UGACCCUGCCGUGUAC | |
| RNACS6299 | TRAC5_ | GCUGGUACACGGCAGGGUCAGUUUUAGAGCUAGAAAUAGCAAGUUAAAAUAAGG | 17454 |
| | P12R16_del5- | CUAGUCCGUUAUCAACUUGAAAAAGUGGCACCGAGUCGGUGCCACAGAUAUCCAG | |
| | PAM | UGACCCUGCCGUGUA | |
| RNACS6300 | TRAC5_ | GCUGGUACACGGCAGGGUCAGUUUUAGAGCUAGAAAUAGCAAGUUAAAAUAAGG | 17455 |
| | P11R16_del5- | CUAGUCCGUUAUCAACUUGAAAAAGUGGCACCGAGUCGGUGCCACAGAUAUCCAG | |
| | PAM | UGACCCUGCCGUGU | |
| RNACS6301 | TRAC5_ | GCUGGUACACGGCAGGGUCAGUUUUAGAGCUAGAAAUAGCAAGUUAAAAUAAGG | 17456 |
| | P10R16_del5- | CUAGUCCGUUAUCAACUUGAAAAAGUGGCACCGAGUCGGUGCCACAGAUAUCCAG | |
| | PAM | UGACCCUGCCGUG | |
| RNACS6302 | TRAC5_ | GCUGGUACACGGCAGGGUCAGUUUUAGAGCUAGAAAUAGCAAGUUAAAAUAAGG | 17457 |
| | P9R16_del5- | CUAGUCCGUUAUCAACUUGAAAAAGUGGCACCGAGUCGGUGCCACAGAUAUCCAG | |
| | PAM | UGACCCUGCCGU | |
| RNACS6303 | TRAC5_ | GCUGGUACACGGCAGGGUCAGUUUUAGAGCUAGAAAUAGCAAGUUAAAAUAAGG | 17458 |
| | P8R16_del5- | CUAGUCCGUUAUCAACUUGAAAAAGUGGCACCGAGUCGGUGCCACAGAUAUCCAG | |
| | PAM | UGACCCUGCCG | |
| RNACS6304 | TRAC5_ | GCUGGUACACGGCAGGGUCAGUUUUAGAGCUAGAAAUAGCAAGUUAAAAUAAGG | 17459 |
| | P13R18_del5- | CUAGUCCGUUAUCAACUUGAAAAAGUGGCACCGAGUCGGUGCCCCACAGAUAUCC | |
| | PAM | AGUGACCCUGCCGUGUAC | |
| RNACS6305 | TRAC5_ | GCUGGUACACGGCAGGGUCAGUUUUAGAGCUAGAAAUAGCAAGUUAAAAUAAGG | 17460 |
| | P12R18_del5- | CUAGUCCGUUAUCAACUUGAAAAAGUGGCACCGAGUCGGUGCCCCACAGAUAUCC | |
| | PAM | AGUGACCCUGCCGUGUA | |
| RNACS6306 | TRAC5_ | GCUGGUACACGGCAGGGUCAGUUUUAGAGCUAGAAAUAGCAAGUUAAAAUAAGG | 17461 |
| | P11R18_del5- | CUAGUCCGUUAUCAACUUGAAAAAGUGGCACCGAGUCGGUGCCCCACAGAUAUCC | |
| | PAM | AGUGACCCUGCCGUGU | |
| RNACS6307 | TRAC5_ | GCUGGUACACGGCAGGGUCAGUUUUAGAGCUAGAAAUAGCAAGUUAAAAUAAGG | 17462 |
| | P10R18_del5- | CUAGUCCGUUAUCAACUUGAAAAAGUGGCACCGAGUCGGUGCCCCACAGAUAUCC | |
| | PAM | AGUGACCCUGCCGUG | |
| RNACS6308 | TRAC5_ | GCUGGUACACGGCAGGGUCAGUUUUAGAGCUAGAAAUAGCAAGUUAAAAUAAGG | 17463 |
| | P9R18_del5- | CUAGUCCGUUAUCAACUUGAAAAAGUGGCACCGAGUCGGUGCCCCACAGAUAUCC | |
| | PAM | AGUGACCCUGCCGU | |
| RNACS6309 | TRAC5_ | GCUGGUACACGGCAGGGUCAGUUUUAGAGCUAGAAAUAGCAAGUUAAAAUAAGG | 17464 |
| | P8R18_del5- | CUAGUCCGUUAUCAACUUGAAAAAGUGGCACCGAGUCGGUGCCCCACAGAUAUCC | |
| | PAM | AGUGACCCUGCCG | |
| RNACS6310 | TRAC5_ | GCUGGUACACGGCAGGGUCAGUUUUAGAGCUAGAAAUAGCAAGUUAAAAUAAGG | 17465 |
| | P13R20_del5- | CUAGUCCGUUAUCAACUUGAAAAAGUGGCACCGAGUCGGUGCGUCCCACAGAUAU | |
| | PAM | CCAGUGACCCUGCCGUGUAC | |
| RNACS6311 | TRAC5_ | GCUGGUACACGGCAGGGUCAGUUUUAGAGCUAGAAAUAGCAAGUUAAAAUAAGG | 17466 |
| | P12R20_del5- | CUAGUCCGUUAUCAACUUGAAAAAGUGGCACCGAGUCGGUGCGUCCCACAGAUAU | |
| | PAM | CCAGUGACCCUGCCGUGUA | |
| RNACS6312 | TRAC5_ | GCUGGUACACGGCAGGGUCAGUUUUAGAGCUAGAAAUAGCAAGUUAAAAUAAGG | 17467 |
| | P11R20_del5- | CUAGUCCGUUAUCAACUUGAAAAAGUGGCACCGAGUCGGUGCGUCCCACAGAUAU | |
| | PAM | CCAGUGACCCUGCCGUGU | |
| RNACS6313 | TRAC5_ | GCUGGUACACGGCAGGGUCAGUUUUAGAGCUAGAAAUAGCAAGUUAAAAUAAGG | 17468 |
| | P10R20_del5- | CUAGUCCGUUAUCAACUUGAAAAAGUGGCACCGAGUCGGUGCGUCCCACAGAUAU | |
| | PAM | CCAGUGACCCUGCCGUG | |
| RNACS6314 | TRAC5_ | GCUGGUACACGGCAGGGUCAGUUUUAGAGCUAGAAAUAGCAAGUUAAAAUAAGG | 17469 |
| | P9R20_del5- | CUAGUCCGUUAUCAACUUGAAAAAGUGGCACCGAGUCGGUGCGUCCCACAGAUAU | |
| | PAM | CCAGUGACCCUGCCGU | |
| RNACS6315 | TRAC5_ | GCUGGUACACGGCAGGGUCAGUUUUAGAGCUAGAAAUAGCAAGUUAAAAUAAGG | 17470 |
| | P8R20_del5- | CUAGUCCGUUAUCAACUUGAAAAAGUGGCACCGAGUCGGUGCGUCCCACAGAUAU | |
| | PAM | CCAGUGACCCUGCCG | |
| RNACS6316 | TRAC5_ | GCUGGUACACGGCAGGGUCAGUUUUAGAGCUAGAAAUAGCAAGUUAAAAUAAGG | 17471 |
| | P13R8_del7- | CUAGUCCGUUAUCAACUUGAAAAAGUGGCACCGAGUCGGUGCUAUCCUGACCCUG | |
| | PAM | CCGUGUAC | |
| RNACS6317 | TRAC5_ | GCUGGUACACGGCAGGGUCAGUUUUAGAGCUAGAAAUAGCAAGUUAAAAUAAGG | 17472 |
| | P12R8_del7- | CUAGUCCGUUAUCAACUUGAAAAAGUGGCACCGAGUCGGUGCUAUCCUGACCCUG | |
| | PAM | CCGUGUA | |
| RNACS6318 | TRAC5_ | GCUGGUACACGGCAGGGUCAGUUUUAGAGCUAGAAAUAGCAAGUUAAAAUAAGG | 17473 |
| | P11R8_del7- | CUAGUCCGUUAUCAACUUGAAAAAGUGGCACCGAGUCGGUGCUAUCCUGACCCUG | |
| | PAM | CCGUGU | |
| RNACS6319 | TRAC5_ | GCUGGUACACGGCAGGGUCAGUUUUAGAGCUAGAAAUAGCAAGUUAAAAUAAGG | 17474 |
| | P10R8_del7- | CUAGUCCGUUAUCAACUUGAAAAAGUGGCACCGAGUCGGUGCUAUCCUGACCCUG | |
| | PAM | CCGUG | |
| RNACS6320 | TRAC5_ | GCUGGUACACGGCAGGGUCAGUUUUAGAGCUAGAAAUAGCAAGUUAAAAUAAGG | 17475 |
| | P9R8_del7- | CUAGUCCGUUAUCAACUUGAAAAAGUGGCACCGAGUCGGUGCUAUCCUGACCCUG | |
| | PAM | CCGU | |
| RNACS6321 | TRAC5_ | GCUGGUACACGGCAGGGUCAGUUUUAGAGCUAGAAAUAGCAAGUUAAAAUAAGG | 17476 |
| | P8R8_del7- | CUAGUCCGUUAUCAACUUGAAAAAGUGGCACCGAGUCGGUGCUAUCCUGACCCUG | |
| | PAM | CCG | |
| RNACS6322 | TRAC5_ | GCUGGUACACGGCAGGGUCAGUUUUAGAGCUAGAAAUAGCAAGUUAAAAUAAGG | 17477 |
| | P13R10_del7- | CUAGUCCGUUAUCAACUUGAAAAAGUGGCACCGAGUCGGUGCGAUAUCCUGACCC | |
| | PAM | UGCCGUGUAC | |
| RNACS6323 | TRAC5_ | GCUGGUACACGGCAGGGUCAGUUUUAGAGCUAGAAAUAGCAAGUUAAAAUAAGG | 17478 |
| | P12R10_del7- | CUAGUCCGUUAUCAACUUGAAAAAGUGGCACCGAGUCGGUGCGAUAUCCUGACCC | |
| | PAM | UGCCGUGUA | |
| RNACS6324 | TRAC5_ | GCUGGUACACGGCAGGGUCAGUUUUAGAGCUAGAAAUAGCAAGUUAAAAUAAGG | 17479 |
| | P11R10_del7- | CUAGUCCGUUAUCAACUUGAAAAAGUGGCACCGAGUCGGUGCGAUAUCCUGACCC | |
| | PAM | UGCCGUGU | |
| RNACS6325 | TRAC5_ | GCUGGUACACGGCAGGGUCAGUUUUAGAGCUAGAAAUAGCAAGUUAAAAUAAGG | 17480 |
| | P10R10_del7- | CUAGUCCGUUAUCAACUUGAAAAAGUGGCACCGAGUCGGUGCGAUAUCCUGACCC | |
| | PAM | UGCCGUG | |
| RNACS6326 | TRAC5_ | GCUGGUACACGGCAGGGUCAGUUUUAGAGCUAGAAAUAGCAAGUUAAAAUAAGG | 17481 |
| | P9R10_del7- | CUAGUCCGUUAUCAACUUGAAAAAGUGGCACCGAGUCGGUGCGAUAUCCUGACCC | |
| | PAM | UGCCGU | |
| RNACS6327 | TRAC5_ | GCUGGUACACGGCAGGGUCAGUUUUAGAGCUAGAAAUAGCAAGUUAAAAUAAGG | 17482 |
| | P8R10_del7- | CUAGUCCGUUAUCAACUUGAAAAAGUGGCACCGAGUCGGUGCGAUAUCCUGACCC | |
| | PAM | UGCCG | |
| RNACS6328 | TRAC5_ | GCUGGUACACGGCAGGGUCAGUUUUAGAGCUAGAAAUAGCAAGUUAAAAUAAGG | 17483 |
| | P13R12_del7- | CUAGUCCGUUAUCAACUUGAAAAAGUGGCACCGAGUCGGUGCCAGAUAUCCUGAC | |
| | PAM | CCUGCCGUGUAC | |
| RNACS6329 | TRAC5_ | GCUGGUACACGGCAGGGUCAGUUUUAGAGCUAGAAAUAGCAAGUUAAAAUAAGG | 17484 |
| | P12R12_del7- | CUAGUCCGUUAUCAACUUGAAAAAGUGGCACCGAGUCGGUGCCAGAUAUCCUGAC | |
| | PAM | CCUGCCGUGUA | |
| RNACS6330 | TRAC5_ | GCUGGUACACGGCAGGGUCAGUUUUAGAGCUAGAAAUAGCAAGUUAAAAUAAGG | 17485 |
| | P11R12_del7- | CUAGUCCGUUAUCAACUUGAAAAAGUGGCACCGAGUCGGUGCCAGAUAUCCUGAC | |
| | PAM | CCUGCCGUGU | |
| RNACS6331 | TRAC5_ | GCUGGUACACGGCAGGGUCAGUUUUAGAGCUAGAAAUAGCAAGUUAAAAUAAGG | 17486 |
| | P10R12_del7- | CUAGUCCGUUAUCAACUUGAAAAAGUGGCACCGAGUCGGUGCCAGAUAUCCUGAC | |
| | PAM | CCUGCCGUG | |
| RNACS6332 | TRAC5_ | GCUGGUACACGGCAGGGUCAGUUUUAGAGCUAGAAAUAGCAAGUUAAAAUAAGG | 17487 |
| | P9R12_del7- | CUAGUCCGUUAUCAACUUGAAAAAGUGGCACCGAGUCGGUGCCAGAUAUCCUGAC | |
| | PAM | CCUGCCGU | |
| RNACS6333 | TRAC5_ | GCUGGUACACGGCAGGGUCAGUUUUAGAGCUAGAAAUAGCAAGUUAAAAUAAGG | 17488 |
| | P8R12_del7- | CUAGUCCGUUAUCAACUUGAAAAAGUGGCACCGAGUCGGUGCCAGAUAUCCUGAC | |
| | PAM | CCUGCCG | |
| RNACS6334 | TRAC5_ | GCUGGUACACGGCAGGGUCAGUUUUAGAGCUAGAAAUAGCAAGUUAAAAUAAGG | 17489 |
| | P13R14_del7- | CUAGUCCGUUAUCAACUUGAAAAAGUGGCACCGAGUCGGUGCCACAGAUAUCCUG | |
| | PAM | ACCCUGCCGUGUAC | |
| RNACS6335 | TRAC5_ | GCUGGUACACGGCAGGGUCAGUUUUAGAGCUAGAAAUAGCAAGUUAAAAUAAGG | 17490 |
| | P12R14_del7- | CUAGUCCGUUAUCAACUUGAAAAAGUGGCACCGAGUCGGUGCCACAGAUAUCCUG | |
| | PAM | ACCCUGCCGUGUA | |
| RNACS6336 | TRAC5_ | GCUGGUACACGGCAGGGUCAGUUUUAGAGCUAGAAAUAGCAAGUUAAAAUAAGG | 17491 |
| | P11R14_del7- | CUAGUCCGUUAUCAACUUGAAAAAGUGGCACCGAGUCGGUGCCACAGAUAUCCUG | |
| | PAM | ACCCUGCCGUGU | |
| RNACS6337 | TRAC5_ | GCUGGUACACGGCAGGGUCAGUUUUAGAGCUAGAAAUAGCAAGUUAAAAUAAGG | 17492 |
| | P10R14_del7- | CUAGUCCGUUAUCAACUUGAAAAAGUGGCACCGAGUCGGUGCCACAGAUAUCCUG | |
| | PAM | ACCCUGCCGUG | |
| RNACS6338 | TRAC5_ | GCUGGUACACGGCAGGGUCAGUUUUAGAGCUAGAAAUAGCAAGUUAAAAUAAGG | 17493 |
| | P9R14_del7- | CUAGUCCGUUAUCAACUUGAAAAAGUGGCACCGAGUCGGUGCCACAGAUAUCCUG | |
| | PAM | ACCCUGCCGU | |
| RNACS6339 | TRAC5_ | GCUGGUACACGGCAGGGUCAGUUUUAGAGCUAGAAAUAGCAAGUUAAAAUAAGG | 17494 |
| | P8R14_del7- | CUAGUCCGUUAUCAACUUGAAAAAGUGGCACCGAGUCGGUGCCACAGAUAUCCUG | |
| | PAM | ACCCUGCCG | |
| RNACS6340 | TRAC5_ | GCUGGUACACGGCAGGGUCAGUUUUAGAGCUAGAAAUAGCAAGUUAAAAUAAGG | 17495 |
| | P13R16_del7- | CUAGUCCGUUAUCAACUUGAAAAAGUGGCACCGAGUCGGUGCCCCACAGAUAUCC | |
| | PAM | UGACCCUGCCGUGUAC | |
| RNACS6341 | TRAC5_ | GCUGGUACACGGCAGGGUCAGUUUUAGAGCUAGAAAUAGCAAGUUAAAAUAAGG | 17496 |
| | P12R16_del7- | CUAGUCCGUUAUCAACUUGAAAAAGUGGCACCGAGUCGGUGCCCCACAGAUAUCC | |
| | PAM | UGACCCUGCCGUGUA | |
| RNACS6342 | TRAC5_ | GCUGGUACACGGCAGGGUCAGUUUUAGAGCUAGAAAUAGCAAGUUAAAAUAAGG | 17497 |
| | P11R16_del7- | CUAGUCCGUUAUCAACUUGAAAAAGUGGCACCGAGUCGGUGCCCCACAGAUAUCC | |
| | PAM | UGACCCUGCCGUGU | |
| RNACS6343 | TRAC5_ | GCUGGUACACGGCAGGGUCAGUUUUAGAGCUAGAAAUAGCAAGUUAAAAUAAGG | 17498 |
| | P10R16_del7- | CUAGUCCGUUAUCAACUUGAAAAAGUGGCACCGAGUCGGUGCCCCACAGAUAUCC | |
| | PAM | UGACCCUGCCGUG | |
| RNACS6344 | TRAC5_ | GCUGGUACACGGCAGGGUCAGUUUUAGAGCUAGAAAUAGCAAGUUAAAAUAAGG | 17499 |
| | P9R16_del7- | CUAGUCCGUUAUCAACUUGAAAAAGUGGCACCGAGUCGGUGCCCCACAGAUAUCC | |
| | PAM | UGACCCUGCCGU | |
| RNACS6345 | TRAC5_ | GCUGGUACACGGCAGGGUCAGUUUUAGAGCUAGAAAUAGCAAGUUAAAAUAAGG | 17500 |
| | P8R16_del7- | CUAGUCCGUUAUCAACUUGAAAAAGUGGCACCGAGUCGGUGCCCCACAGAUAUCC | |
| | PAM | UGACCCUGCCG | |
| RNACS6346 | TRAC5_ | GCUGGUACACGGCAGGGUCAGUUUUAGAGCUAGAAAUAGCAAGUUAAAAUAAGG | 17501 |
| | P13R18_del7- | CUAGUCCGUUAUCAACUUGAAAAAGUGGCACCGAGUCGGUGCGUCCCACAGAUAU | |
| | PAM | CCUGACCCUGCCGUGUAC | |
| RNACS6347 | TRAC5_ | GCUGGUACACGGCAGGGUCAGUUUUAGAGCUAGAAAUAGCAAGUUAAAAUAAGG | 17502 |
| | P12R18_del7- | CUAGUCCGUUAUCAACUUGAAAAAGUGGCACCGAGUCGGUGCGUCCCACAGAUAU | |
| | PAM | CCUGACCCUGCCGUGUA | |
| RNACS6348 | TRAC5_ | GCUGGUACACGGCAGGGUCAGUUUUAGAGCUAGAAAUAGCAAGUUAAAAUAAGG | 17503 |
| | P11R18_del7- | CUAGUCCGUUAUCAACUUGAAAAAGUGGCACCGAGUCGGUGCGUCCCACAGAUAU | |
| | PAM | CCUGACCCUGCCGUGU | |
| RNACS6349 | TRAC5_ | GCUGGUACACGGCAGGGUCAGUUUUAGAGCUAGAAAUAGCAAGUUAAAAUAAGG | 17504 |
| | P10R18_del7- | CUAGUCCGUUAUCAACUUGAAAAAGUGGCACCGAGUCGGUGCGUCCCACAGAUAU | |
| | PAM | CCUGACCCUGCCGUG | |
| RNACS6350 | TRAC5_ | GCUGGUACACGGCAGGGUCAGUUUUAGAGCUAGAAAUAGCAAGUUAAAAUAAGG | 17505 |
| | P9R18_del7- | CUAGUCCGUUAUCAACUUGAAAAAGUGGCACCGAGUCGGUGCGUCCCACAGAUAU | |
| | PAM | CCUGACCCUGCCGU | |
| RNACS6351 | TRAC5_ | GCUGGUACACGGCAGGGUCAGUUUUAGAGCUAGAAAUAGCAAGUUAAAAUAAGG | 17506 |
| | P8R18_del7- | CUAGUCCGUUAUCAACUUGAAAAAGUGGCACCGAGUCGGUGCGUCCCACAGAUAU | |
| | PAM | CCUGACCCUGCCG | |
| RNACS6352 | TRAC5_ | GCUGGUACACGGCAGGGUCAGUUUUAGAGCUAGAAAUAGCAAGUUAAAAUAAGG | 17507 |
| | P13R20_del7- | CUAGUCCGUUAUCAACUUGAAAAAGUGGCACCGAGUCGGUGCUUGUCCCACAGAU | |
| | PAM | AUCCUGACCCUGCCGUGUAC | |
| RNACS6353 | TRAC5_ | GCUGGUACACGGCAGGGUCAGUUUUAGAGCUAGAAAUAGCAAGUUAAAAUAAGG | 17508 |
| | P12R20_del7- | CUAGUCCGUUAUCAACUUGAAAAAGUGGCACCGAGUCGGUGCUUGUCCCACAGAU | |
| | PAM | AUCCUGACCCUGCCGUGUA | |
| RNACS6354 | TRAC5_ | GCUGGUACACGGCAGGGUCAGUUUUAGAGCUAGAAAUAGCAAGUUAAAAUAAGG | 17509 |
| | P11R20_del7- | CUAGUCCGUUAUCAACUUGAAAAAGUGGCACCGAGUCGGUGCUUGUCCCACAGAU | |
| | PAM | AUCCUGACCCUGCCGUGU | |
| RNACS6355 | TRAC5_ | GCUGGUACACGGCAGGGUCAGUUUUAGAGCUAGAAAUAGCAAGUUAAAAUAAGG | 17510 |
| | P10R20_del7- | CUAGUCCGUUAUCAACUUGAAAAAGUGGCACCGAGUCGGUGCUUGUCCCACAGAU | |
| | PAM | AUCCUGACCCUGCCGUG | |
| RNACS6356 | TRAC5_ | GCUGGUACACGGCAGGGUCAGUUUUAGAGCUAGAAAUAGCAAGUUAAAAUAAGG | 17511 |
| | P9R20_del7- | CUAGUCCGUUAUCAACUUGAAAAAGUGGCACCGAGUCGGUGCUUGUCCCACAGAU | |
| | PAM | AUCCUGACCCUGCCGU | |
| RNACS6357 | TRAC5_ | GCUGGUACACGGCAGGGUCAGUUUUAGAGCUAGAAAUAGCAAGUUAAAAUAAGG | 17512 |
| | P8R20_del7- | CUAGUCCGUUAUCAACUUGAAAAAGUGGCACCGAGUCGGUGCUUGUCCCACAGAU | |
| | PAM | AUCCUGACCCUGCCG | |

In some embodiments, the template RNA comprises a sequence of a gRNA spacer, gRNA scaffold, 5′RT template sequence, insert, and PBS sequence of Table 3A. In some embodiments, the template RNA comprises a sequence of a gRNA spacer, gRNA scaffold, 5′RT template sequence, insert, 3′RT template sequence, and PBS sequence of Table 3A.

TABLE 3A
Exemplary Template RNA sequences comprising TRAC4 or TRAC5 gRNA spacers
Table 3A provides components of template RNAs comprising the TRAC4 gRNA spacer
(gagaaucaaaaucggugaau SEQ ID NO. 17513) or TRAC5 gRNA spacer (gcugguacacggcaggguca
SEQ ID NO. 17514) for making an edit within the TRAC gene either at a nick site or a PAM
site. The sequences provided include gRNA spacers, gRNA scaffolds, 5′ RT template sequences,
5-nucleotide insert sequences, optional 3′ RT template sequences, and PBS sequences.

| | | | | SEQ | 5′ RT | SEQ | | 3′ RT | | SEQ |
|---|---|---|---|---|---|---|---|---|---|---|
| | Location | gRNA | gRNA | ID | Template | ID | | template | | ID |
| Name | of edit | Spacer | Scaffold | NO | Sequence | NO | insert | sequence | PBS | NO |
| TRAC5 | at nick | Gcugguacac | GuuuuAGAGCu | 17515 | aucauguccuaa | 17516 | NNNNN | | ccugcc | 17520 |
| | | ggcaggguca | AGAAAuAGCA | | cccugauccucu | | | | guguac | |
| | | SEQ ID NO. | AGuuAAAAuA | | ugucccacagau | | | | cagc | |
| | | 17514 | AGGCuAGuCC | | auccagaacccu | | | | | |
| | | | GuuAuCAACuu | | ga | | | | | |
| | | | GAAAAAGuGG | | | | | | | |
| | | | CACCGAGuCG | | | | | | | |
| | | | GuGC | | | | | | | |
| TRAC5 | at PAM | gcugguacac | GuuuuAGAGCu | 17515 | aucauguccuaa | 17517 | NNNNN | uga | ccugcc | 17520 |
| | | ggcaggguca | AGAAAuAGCA | | cccugauccucu | | | | guguac | |
| | | SEQ ID NO. | AGuuAAAAuA | | ugucccacagau | | | | cagc | |
| | | 17514 | AGGCuAGuCC | | auccagaaccc | | | | | |
| | | | GuuAuCAACuu | | | | | | | |
| | | | GAAAAAGuGG | | | | | | | |
| | | | CACCGAGuCG | | | | | | | |
| | | | GuGC | | | | | | | |
| TRAC4 | at nick | gagaaucaaa | GuuuuAGAGCu | 17515 | guguaccagcug | 17518 | NNNNN | | caccga | 17521 |
| | | aucggugaau | AGAAAuAGCA | | agagacucuaaa | | | | uuuuga | |
| | | SEQ ID NO. | AGuuAAAAuA | | uccagugacaag | | | | uucuc | |
| | | 17513 | AGGCuAGuCC | | ucugucugccua | | | | | |
| | | | GuuAuCAACuu | | uu | | | | | |
| | | | GAAAAAGuGG | | | | | | | |
| | | | CACCGAGuCG | | | | | | | |
| | | | GuGC | | | | | | | |
| TRAC4 | at PAM | gagaaucaaa | GuuuuAGAGCu | 17515 | guguaccagcug | 17519 | NNNNN | auu | caccga | 17521 |
| | | aucggugaau | AGAAAuAGCA | | agagacucuaaa | | | | uuuuga | |
| | | SEQ ID NO. | AGuuAAAAuA | | uccagugacaag | | | | uucuc | |
| | | 17513 | AGGCuAGuCC | | ucugucugccu | | | | | |
| | | | GuuAuCAACuu | | | | | | | |
| | | | GAAAAAGuGG | | | | | | | |
| | | | CACCGAGuCG | | | | | | | |
| | | | GuGC | | | | | | | |

The sequences within Table 3A provide the suitable versions of the RT template sequence and PBS sequences for use in the template RNA. Other suitable versions may comprise shorter sequences compared to the sequences shown in Table 3A. In some embodiments, the 5′ RT template sequence can be varied from 0 to 47 nt for an “at PAM” edit, and 0 to 50 for an “at nick” edit. Consequently, in some embodiments, a template RNA described herein comprises a 5′ RT sequence of Table 3A or a portion thereof (e.g., a 3′ portion of the 5′ RT sequence). In some embodiments, where the 5′ RT template sequences comprises fewer bases than shown in Table 3A, bases are removed from the 5′ end. The insert sequences in Table 3A are shown simply with “N” placeholders to signify the ability to use any combination of 5 nucleotides within the template RNA. By way of example, Table 4A provides exemplary 5-nucleotide insertion sequences for use within a template RNA sequence described herein, e.g., the template RNA sequences of Table 3A. In some embodiments, the PBS sequence can be varied from 5 to 17 nucleotides. In some embodiments, where the PBS sequences comprises fewer bases than shown in Table 3A, bases are sequentially removed from the 3′ end. Consequently, in some embodiments, a template RNA described herein comprises a PBS sequence of Table 3A or a portion thereof (e.g., a 5′ portion of the PBS sequence).

TABLE 4A
Exemplary Insert sequences
Table 4A provides exemplary 5 nucleotide insert sequences
for use with template RNAs described herein.

| | | | | | | | | | | |
|---|---|---|---|---|---|---|---|---|---|---|
| AAAAA | ACGCG | AUAUA | CAUAG | CGCGA | CUUUG | GCGCA | GUAGG | UAUAA | UGCCG | UUUUA |
| AAAAC | ACGCU | AUAUC | CAUAU | CGCGC | CUUUU | GCGCC | GUAGU | UAUAC | UGCCU | UUUUC |
| AAAAG | ACGGA | AUAUG | CAUCA | CGCGG | GAAAA | GCGCG | GUAUA | UAUAG | UGCGA | UUUUG |
| AAAAU | ACGGC | AUAUU | CAUCC | CGCGU | GAAAC | GCGCU | GUAUC | UAUAU | UGCGC | UUUUU |
| AAACA | ACGGG | AUCAA | CAUCG | CGCUA | GAAAG | GCGGA | GUAUG | UAUCA | UGCGG | |
| AAACC | ACGGU | AUCAC | CAUCU | CGCUC | GAAAU | GCGGC | GUAUU | UAUCC | UGCGU | |
| AAACG | ACGUA | AUCAG | CAUGA | CGCUG | GAACA | GCGGG | GUCAA | UAUCG | UGCUA | |
| AAACU | ACGUC | AUCAU | CAUGC | CGCUU | GAACC | GCGGU | GUCAC | UAUCU | UGCUC | |
| AAAGA | ACGUG | AUCCA | CAUGG | CGGAA | GAACG | GCGUA | GUCAG | UAUGA | UGCUG | |
| AAAGC | ACGUU | AUCCC | CAUGU | CGGAC | GAACU | GCGUC | GUCAU | UAUGC | UGCUU | |
| AAAGG | ACUAA | AUCCG | CAUUA | CGGAG | GAAGA | GCGUG | GUCCA | UAUGG | UGGAA | |
| AAAGU | ACUAC | AUCCU | CAUUC | CGGAU | GAAGC | GCGLU | GUCCC | UAUGU | UGGAC | |
| AAAUA | ACUAG | AUCGA | CAUUG | CGGCA | GAAGG | GCUAA | GUCCG | UAUUA | UGGAG | |
| AAAUC | ACUAU | AUCGC | CAUUU | CGGCC | GAAGU | GCUAC | GUCCU | UAUUC | UGGAU | |
| AAAUG | ACUCA | AUCGG | CCAAA | CGGCG | GAAUA | GCUAG | GUCGA | UAUUG | UGGCA | |
| AAAUU | ACUCC | AUCGU | CCAAC | CGGCU | GAAUC | GCUAU | GUCGC | UAUUU | UGGCC | |
| AACAA | ACUCG | AUCUA | CCAAG | CGGGA | GAAUG | GCUCA | GUCGG | UCAAA | UGGCG | |
| AACAC | ACUCU | AUCUC | CCAAU | CGGGC | GAAUU | GCUCC | GUCGU | UCAAC | UGGCU | |
| AACAG | ACUGA | AUCUG | CCACA | CGGGG | GACAA | GCUCG | GUCUA | UCAAG | UGGGA | |
| AACAU | ACUGC | AUCUU | CCACC | CGGGU | GACAC | GCUCU | GUCUC | UCAAU | UGGGC | |
| AACCA | ACUGG | AUGAA | CCACG | CGGUA | GACAG | GCUGA | GUCUG | UCACA | UGGGG | |
| AACCC | ACUGU | AUGAC | CCACU | CGGUC | GACAU | GCUGC | GUCUU | UCACC | UGGGU | |
| AACCG | ACUUA | AUGAG | CCAGA | CGGUG | GACCA | GCUGG | GUGAA | UCACG | UGGUA | |
| AACCU | ACUUC | AUGAU | CCAGC | CGGUU | GACCC | GCUGU | GUGAC | UCACU | UGGUC | |
| AACGA | ACUUG | AUGCA | CCAGG | CGUAA | GACCG | GCUCA | GUGAG | UCAGA | UGGUG | |
| AACGC | ACUUU | AUGCC | CCAGU | CGUAC | GACCU | GCUUC | GUGAU | UCAGC | UGGUU | |
| AACGG | AGAAA | AUGCG | CCAUA | CGUAG | GACGA | GCUUG | GUGCA | UCAGG | UGUAA | |
| AACGU | AGAAC | AUGCU | CCAUC | CGUAU | GACGC | GCUUU | GUGCC | UCAGU | UGUAC | |
| AACUA | AGAAG | AUGGA | CCAUG | CGUCA | GACGG | GGAAA | GUGCG | UCAUA | UGUAG | |
| AACUC | AGAAU | AUGGC | CCAUU | CGUCC | GACGU | GGAAC | GUGCU | UCAUC | UGUAU | |
| AACUG | AGACA | AUGGG | CCCAA | CGUCG | GACUA | GGAAG | GUGGA | UCAUG | UGUCA | |
| AACUU | AGACC | AUGGU | CCCAC | CGUCU | GACUC | GGAAU | GUGGC | UCAUU | UGUCC | |
| AAGAA | AGACG | AUGUA | CCCAG | CGUGA | GACUG | GGACA | GUGGG | UCCAA | UGUCG | |
| AAGAC | AGACU | AUGUC | CCCAU | CGUGC | GACUU | GGACC | GUGGU | UCCAC | UGUCU | |
| AAGAG | AGAGA | AUGUG | CCCCA | CGUGG | GAGAA | GGACG | GUGUA | UCCAG | UGUGA | |
| AAGAU | AGAGC | AUGUU | CCCCC | CGUGU | GAGAC | GGACU | GUGUC | UCCAU | UGUGC | |
| AAGCA | AGAGG | AUUAA | CCCCG | CGUUA | GAGAG | GGAGA | GUGUG | UCCCA | UGUGG | |
| AAGCC | AGAGU | AUUAC | CCCCU | CGUUC | GAGAU | GGAGC | GUGUU | UCCCC | UGUGU | |
| AAGCG | AGAUA | AUUAG | CCCGA | CGUUG | GAGCA | GGAGG | GUUAA | UCCCG | UGUUA | |
| AAGCU | AGAUC | AUUAU | CCCGC | CGUUU | GAGCC | GGAGU | GUUAC | UCCCU | UGUUC | |
| AAGGA | AGAUG | AUUCA | CCCGG | CUAAA | GAGCG | GGAUA | GUUAG | UCCGA | UGUUG | |
| AAGGC | AGAUU | AUUCC | CCCGU | CUAAC | GAGCU | GGAUC | GUUAU | UCCGC | UGUUU | |
| AAGGG | AGCAA | AUUCG | CCCUA | CUAAG | GAGGA | GGAUG | GUUCA | UCCGG | UUAAA | |
| AAGGU | AGCAC | AUUCU | CCCUC | CUAAU | GAGGC | GGAUU | GUUCC | UCCGU | UUAAC | |
| AAGUA | AGCAG | AUUGA | CCCUG | CUACA | GAGGG | GGCAA | GUUCG | UCCUA | UUAAG | |
| AAGUC | AGCAU | AUUGC | CCCUU | CUACC | GAGGU | GGCAC | GUUCU | UCCUC | UUAAU | |
| AAGUG | AGCCA | AUUGG | CCGAA | CUACG | GAGUA | GGCAG | GUUGA | UCCUG | UUACA | |
| AAGUU | AGCCC | AUUGU | CCGAC | CUACU | GAGUC | GGCAU | GUUGC | UCCUU | UUACC | |
| AAUAA | AGCCG | AUUUA | CCGAG | CUAGA | GAGUG | GGCCA | GUUGG | UCGAA | UUACG | |
| AAUAC | AGCCU | AUUUC | CCGAU | CUAGC | GAGUU | GGCCC | GUUGU | UCGAC | UUACU | |
| AAUAG | AGCGA | AUUUG | CCGCA | CUAGG | GAUAA | GGCCG | GUUUA | UCGAG | UUAGA | |
| AAUAU | AGCGC | AUUUU | CCGCC | CUAGU | GAUAC | GGCCU | GUUUC | UCGAU | UUAGC | |
| AAUCA | AGCGG | CAAAA | CCGCG | CUAUA | GAUAG | GGCGA | GUUUG | UCGCA | UUAGG | |
| AAUCC | AGCGU | CAAAC | CCGCU | CUAUC | GAUAU | GGCGC | GUUUU | UCGCC | UUAGU | |
| AAUCG | AGCUA | CAAAG | CCGGA | CUAUG | GAUCA | GGCGG | UAAAA | UCGCG | UUAUA | |
| AAUCU | AGCUC | CAAAU | CCGGC | CUAUU | GAUCC | GGCGU | UAAAC | UCGCU | UUAUC | |
| AAUGA | AGCUG | CAACA | CCGGG | CUCAA | GAUCG | GGCUA | UAAAG | UCGGA | UUAUG | |
| AAUGC | AGCUU | CAACC | CCGGU | CUCAC | GAUCU | GGCUC | UAAAU | UCGGC | UUAUU | |
| AAUGG | AGGAA | CAACG | CCGUA | CUCAG | GAUGA | GGCUG | UAACA | UCGGG | UUCAA | |
| AAUGU | AGGAC | CAACU | CCGUC | CUCAU | GAUGC | GGCUU | UAACC | UCGGU | UUCAC | |
| AAUUA | AGGAG | CAAGA | CCGUG | CUCCA | GAUGG | GGGAA | UAACG | UCGUA | UUCAG | |
| AAUUC | AGGAU | CAAGC | CCGUU | CUCCC | GAUGU | GGGAC | UAACU | UCGUC | UUCAU | |
| AAUUG | AGGCA | CAAGG | CCUAA | CUCCG | GAUUA | GGGAG | UAAGA | UCGUG | UUCCA | |
| AAUUU | AGGCC | CAAGU | CCUAC | CUCCU | GAUUC | GGGAU | UAAGC | UCGUU | UUCCC | |
| ACAAA | AGGCG | CAAUA | CCUAG | CUCGA | GAUUG | GGGCA | UAAGG | UCUAA | UUCCG | |
| ACAAC | AGGCU | CAAUC | CCUAU | CUCGC | GAUUU | GGGCC | UAAGU | UCUAC | UUCCU | |
| ACAAG | AGGGA | CAAUG | CCUCA | CUCGG | GCAAA | GGGCG | UAAUA | UCUAG | UUCGA | |
| ACAAU | AGGGC | CAAUU | CCUCC | CUCGU | GCAAC | GGGCU | UAAUC | UCUAU | UUCGC | |
| ACACA | AGGGG | CACAA | CCUCG | CUCUA | GCAAG | GGGGA | UAAUG | UCUCA | UUCGG | |
| ACACC | AGGGU | CACAC | CCUCU | CUCUC | GCAAU | GGGGC | UAAUU | UCUCC | UUCGU | |
| ACACG | AGGUA | CACAG | CCUGA | CUCUG | GCACA | GGGGG | UACAA | UCUCG | UUCUA | |
| ACACU | AGGUC | CACAU | CCUGC | CUCUU | GCACC | GGGGU | UACAC | UCUCU | UUCUC | |
| ACAGA | AGGUG | CACCA | CCUGG | CUGAA | GCACG | GGGUA | UACAG | UCUGA | UUCUG | |
| ACAGC | AGGUU | CACCC | CCUGU | CUGAC | GCACU | GGGUC | UACAU | UCUGC | UUCUU | |
| ACAGG | AGUAA | CACCG | CCUUA | CUGAG | GCAGA | GGGUG | UACCA | UCUGG | UUGAA | |
| ACAGU | AGUAC | CACCU | CCUUC | CUGAU | GCAGC | GGGUU | UACCC | UCUGU | UUGAC | |
| ACAUA | AGUAG | CACGA | CCUUG | CUGCA | GCAGG | GGUAA | UACCG | UCUUA | UUGAG | |
| ACAUC | AGUAU | CACGC | CCUUU | CUGCC | GCAGU | GGUAC | UACCU | UCUUC | UUGAU | |
| ACAUG | AGUCA | CACGG | CGAAA | CUGCG | GCAUA | GGUAG | UACGA | UCUUG | UUGCA | |
| ACAUU | AGUCC | CACGU | CGAAC | CUGCU | GCAUC | GGUAU | UACGC | UCUUU | UUGCC | |
| ACCAA | AGUCG | CACUA | CGAAG | CUGGA | GCAUG | GGUCA | UACGG | UGAAA | UUGCG | |
| ACCAC | AGUCU | CACUC | CGAAU | CUGGC | GCAUU | GGUCC | UACGU | UGAAC | UUGCU | |
| ACCAG | AGUGA | CACUG | CGACA | CUGGG | GCCAA | GGUCG | UACUA | UGAAG | UUGGA | |
| ACCAU | AGUGC | CACUU | CGACC | CUGGU | GCCAC | GGUCU | UACUC | UGAAU | UUGGC | |
| ACCCA | AGUGG | CAGAA | CGACG | CUGUA | GCCAG | GGUGA | UACUG | UGACA | UUGGG | |
| ACCCC | AGUGU | CAGAC | CGACU | CUGUC | GCCAU | GGUGC | UACUU | UGACC | UUGGU | |
| ACCCG | AGUUA | CAGAG | CGAGA | CUGUG | GCCCA | GGUGG | UAGAA | UGACG | UUGUA | |
| ACCCU | AGUUC | CAGAU | CGAGC | CUGUU | GCCCC | GGUGU | UAGAC | UGACU | UUGUC | |
| ACCGA | AGUUG | CAGCA | CGAGG | CUUAA | GCCCG | GGUUA | UAGAG | UGAGA | UUGUG | |
| ACCGC | AGUUU | CAGCC | CGAGU | CUUAC | GCCCU | GGUUC | UAGAU | UGAGC | UUGUU | |
| ACCGG | AUAAA | CAGCG | CGAUA | CUUAG | GCCGA | GGUUG | UAGCA | UGAGG | UUUAA | |
| ACCGU | AUAAC | CAGCU | CGAUC | CUUAU | GCCGC | GGUUU | UAGCC | UGAGU | UUUAC | |
| ACCUA | AUAAG | CAGGA | CGAUG | CUUCA | GCCGG | GUAAA | UAGCG | UGAUA | UUUAG | |
| ACCUC | AUAAU | CAGGC | CGAUU | CUUCC | GCCGU | GUAAC | UAGCU | UGAUC | UUUAU | |
| ACCUG | AUACA | CAGGG | CGCAA | CUUCG | GCCUA | GUAAG | UAGGA | UGAUG | UUUCA | |
| ACCUU | AUACC | CAGGU | CGCAC | CUUCU | GCCUC | GUAAU | UAGGC | UGAUU | UUUCC | |
| ACGAA | AUACG | CAGUA | CGCAG | CUUGA | GCCUG | GUACA | UAGGG | UGCAA | UUUCG | |
| ACGAC | AUACU | CAGUC | CGCAU | CUUGC | GCCUU | GUACC | UAGGU | UGCAC | UUUCU | |
| ACGAG | AUAGA | CAGUG | CGCCA | CUUGG | GCGAA | GUACG | UAGUA | UGCAG | UUUGA | |
| ACGAU | AUAGC | CAGUU | CGCCC | CUUGU | GCGAC | GUACU | UAGUC | UGCAU | UUUGC | |
| ACGCA | AUAGG | CAUAA | CGCCG | CUUUA | GCGAG | GUAGA | UAGUG | UGCCA | UUUGG | |
| ACGCC | AUAGU | CAUAC | CGCCU | CUUUC | GCGAU | GUAGC | UAGUU | UGCCC | UUUGU | |

TABLE 1B
Exemplary Template RNAs and Sequences Table 1B
provides exemplary template RNAs for use in combination
within a heterologous gene modifying system described
herein for disrupting, the B2M gene using a B2M1 gRNA
spacer (acUcacgcUggaUagccUcc SEQ ID NO. 18195) or a
B2M2 gRNA spacer (gagUagcgcgagcacagcUa SEQ ID NO. 18196).
The description of each exemplary template RNA (column 2)
identifies the gRNA spacer, the length of the PBS, the
length of the heterologous object sequence, the nature
and size of the edit, and the location in the target
nucleic acid sequence. For example B2m1_P13R11_ins4_nick_v1
indicates that the template RNA comprised a B2M1 gRNA spacer,
a PBS of length 13 nucleotides, an RT region (heterologous
object sequence and insertion/deletion) of length 11 nucleotides,
and was designed to produce an insertion of 4 nucleotides near
the nick. In the sequences of Table 1B, the gRNA spacer is the
5′-most sequence and has the sequence of B2M1 or B2M2;
immediately 3′ of the gRNA spacer is the gRNA scaffold which
comprises a sequence of SEQ ID NO: 17515; immediately 3′ of
the gRNA scaffold is the heterologous object sequence which
has the length indicated in the description of column 2; and
immediately 3′ of the heterologous sequence is the PBS which
has the length indicated in the description of column 2.
Nucleotide modifications are noted as follows: phosphorothioate
linkages denoted by an asterisk, 2′-O-methyl groups denoted
by an ‘m’ preceding a nucleotide.

| | | | SEQ |
|---|---|---|---|
| | | | ID |
| Identifier | Description | Sequence | NO |
| RNACS7651 | B2m1_ | mA*mC*mU*rCrArCrGrCrUrGrGrArUrArGrCrCrUrCrCrGrUrUrUrUrA | 17523 |
| | P13R11_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrGrGrArUrGrArUrGr | |
| | v1 | GrCrUrArUrCrCrArG*mC*mG*mU | |
| RNACS7652 | B2m1_ | mA*mC*mU*rCrArCrGrCrUrGrGrArUrArGrCrCrUrCrCrGrUrUrUrUrA | 17524 |
| | P12R11_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrGrGrArUrGrArUrGr | |
| | v1 | GrCrUrArUrCrCrA*mG*mC*mG | |
| RNACS7653 | B2m1_ | mA*mC*mU*rCrArCrGrCrUrGrGrArUrArGrCrCrUrCrCrGrUrUrUrUrA | 17525 |
| | P11R11_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrGrGrArUrGrArUrGr | |
| | v1 | GrCrUrArUrCrC*mA*mG*mC | |
| RNACS7654 | B2m1_ | mA*mC*mU*rCrArCrGrCrUrGrGrArUrArGrCrCrUrCrCrGrUrUrUrUrA | 17526 |
| | P10R11_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrGrGrArUrGrArUrGr | |
| | v1 | GrCrUrArUrC*mC*mA*mG | |
| RNACS7655 | B2m1_ | mA*mC*mU*rCrArCrGrCrUrGrGrArUrArGrCrCrUrCrCrGrUrUrUrUrA | 17527 |
| | P9R11_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrGrGrArUrGrArUrGr | |
| | v1 | GrCrUrArU*mC*mC*mA | |
| RNACS7656 | B2m1_ | mA*mC*mU*rCrArCrGrCrUrGrGrArUrArGrCrCrUrCrCrGrUrUrUrUrA | 17528 |
| | P8R11_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrGrGrArUrGrArUrGr | |
| | v1 | GrCrUrA*mU*mC*mC | |
| RNACS7657 | B2m1_ | mA*mC*mU*rCrArCrGrCrUrGrGrArUrArGrCrCrUrCrCrGrUrUrUrUrA | 17529 |
| | P13R13_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrGrGrArUrGr | |
| | v1 | ArUrGrGrCrUrArUrCrCrArG*mC*mG*mU | |
| RNACS7658 | B2m1_ | mA*mC*mU*rCrArCrGrCrUrGrGrArUrArGrCrCrUrCrCrGrUrUrUrUrA | 17530 |
| | P12R13_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrGrGrArUrGrAr | |
| | v1 | UrGrGrCrUrArUrCrCrA*mG*mC*mG | |
| RNACS7659 | B2m1_ | mA*mC*mU*rCrArCrGrCrUrGrGrArUrArGrCrCrUrCrCrGrUrUrUrUrA | 17531 |
| | P11R13_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrGrGrArUrGr | |
| | v1 | ArUrGrGrCrUrArUrCrC*mA*mG*mC | |
| RNACS7660 | B2m1_ | mA*mC*mU*rCrArCrGrCrUrGrGrArUrArGrCrCrUrCrCrGrUrUrUrUrA | 17532 |
| | P10R13_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrGrGrArUrGrAr | |
| | v1 | UrGrGrCrUrArUrC*mC*mA*mG | |
| RNACS7661 | B2m1_ | mA*mC*mU*rCrArCrGrCrUrGrGrArUrArGrCrCrUrCrCrGrUrUrUrUrA | 17533 |
| | P9R13_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrGrGrArUrGrAr | |
| | v1 | UrGrGrCrUrArU*mC*mC*mA | |
| RNACS7662 | B2m1_ | mA*mC*mU*rCrArCrGrCrUrGrGrArUrArGrCrCrUrCrCrGrUrUrUrUrA | 17534 |
| | P8R13_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrGrGrArUrGrAr | |
| | v1 | UrGrGrCrUrA*mU*mC*mC | |
| RNACS7663 | B2m1_ | mA*mC*mU*rCrArCrGrCrUrGrGrArUrArGrCrCrUrCrCrGrUrUrUrUrA | 17535 |
| | P13R15_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrGrGrCrCrUrGrGrArUr | |
| | v1 | GrArUrGrGrCrUrArUrCrCrArG*mC*mG*mU | |
| RNACS7664 | B2m1_ | mA*mC*mU*rCrArCrGrCrUrGrGrArUrArGrCrCrUrCrCrGrUrUrUrUrA | 17536 |
| | P12R15_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrGrGrCrCrUrGrGrAr | |
| | v1 | UrGrArUrGrGrCrUrArUrCrCrA*mG*mC*mG | |
| RNACS7665 | B2m1_ | mA*mC*mU*rCrArCrGrCrUrGrGrArUrArGrCrCrUrCrCrGrUrUrUrUrA | 17537 |
| | P11R15_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrGrGrCrCrUrGrGrArUr | |
| | v1 | GrArUrGrGrCrUrArUrCrC*mA*mG*mC | |
| RNACS7666 | B2m1_ | mA*mC*mU*rCrArCrGrCrUrGrGrArUrArGrCrCrUrCrCrGrUrUrUrUrA | 17538 |
| | P10R15_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrGrGrCrCrUrGrGrArUr | |
| | v1 | GrArUrGrGrCrUrArUrC*mC*mA*mG | |
| RNACS7667 | B2m1_ | mA*mC*mU*rCrArCrGrCrUrGrGrArUrArGrCrCrUrCrCrGrUrUrUrUrA | 17539 |
| | P9R15_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrGrGrCrCrUrGrGrArUr | |
| | v1 | GrArUrGrGrCrUrArU*mC*mC*mA | |
| RNACS7668 | B2m1_ | mA*mC*mU*rCrArCrGrCrUrGrGrArUrArGrCrCrUrCrCrGrUrUrUrUrA | 17540 |
| | P8R15_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrGrGrCrCrUrGrGrArUr | |
| | v1 | GrArUrGrGrCrUrA*mU*mC*mC | |
| RNACS7669 | B2m1_ | mA*mC*mU*rCrArCrGrCrUrGrGrArUrArGrCrCrUrCrCrGrUrUrUrUrA | 17541 |
| | P13R17_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrUrUrCrUrGrGrCrCrUrGrGr | |
| | v1 | ArUrGrArUrGrGrCrUrArUrCrCrArG*mC*mG*mU | |
| RNACS7670 | B2m1_ | mA*mC*mU*rCrArCrGrCrUrGrGrArUrArGrCrCrUrCrCrGrUrUrUrUrA | 17542 |
| | P12R17_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrUrUrCrUrGrGrCrCrUrGrGr | |
| | v1 | ArUrGrArUrGrGrCrUrArUrCrCrA*mG*mC*mG | |
| RNACS7671 | B2m1_ | mA*mC*mU*rCrArCrGrCrUrGrGrArUrArGrCrCrUrCrCrGrUrUrUrUrA | 17543 |
| | P11R17_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrUrUrCrUrGrGrCrCrUrGrGr | |
| | v1 | ArUrGrArUrGrGrCrUrArUrCrC*mA*mG*mC | |
| RNACS7672 | B2m1_ | mA*mC*mU*rCrArCrGrCrUrGrGrArUrArGrCrCrUrCrCrGrUrUrUrUrA | 17544 |
| | P10R17_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrUrUrCrUrGrGrCrCrUrGrGr | |
| | v1 | ArUrGrArUrGrGrCrUrArUrC*mC*mA*mG | |
| RNACS7673 | B2m1_ | mA*mC*mU*rCrArCrGrCrUrGrGrArUrArGrCrCrUrCrCrGrUrUrUrUrA | 17545 |
| | P9R17_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrUrUrCrUrGrGrCrCrUrGrGr | |
| | v1 | ArUrGrArUrGrGrCrUrArU*mC*mC*mA | |
| RNACS7674 | B2m1_ | mA*mC*mU*rCrArCrGrCrUrGrGrArUrArGrCrCrUrCrCrGrUrUrUrUrA | 17546 |
| | P8R17_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrUrUrCrUrGrGrCrCrUrGrGr | |
| | v1 | ArUrGrArUrGrGrCrUrA*mU*mC*mC | |
| RNACS7675 | B2m1_ | mA*mC*mU*rCrArCrGrCrUrGrGrArUrArGrCrCrUrCrCrGrUrUrUrUrA | 17547 |
| | P13R19_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrUrUrCrUrGrGrCrCrUr | |
| | v1 | GrGrArUrGrArUrGrGrCrUrArUrCrCrArG*mC*mG*mU | |
| RNACS7676 | B2m1_ | mA*mC*mU*rCrArCrGrCrUrGrGrArUrArGrCrCrUrCrCrGrUrUrUrUrA | 17548 |
| | P12R19_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrUrUrCrUrGrGrCrCr | |
| | v1 | UrGrGrArUrGrArUrGrGrCrUrArUrCrCrA*mG*mC*mG | |
| RNACS7677 | B2m1_ | mA*mC*mU*rCrArCrGrCrUrGrGrArUrArGrCrCrUrCrCrGrUrUrUrUrA | 17549 |
| | P11R19_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrUrUrCrUrGrGrCrCrUr | |
| | v1 | GrGrArUrGrArUrGrGrCrUrArUrCrC*mA*mG*mC | |
| RNACS7678 | B2m1_ | mA*mC*mU*rCrArCrGrCrUrGrGrArUrArGrCrCrUrCrCrGrUrUrUrUrA | 17550 |
| | P10R19_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrUrUrCrUrGrGrCrCrUr | |
| | v1 | GrGrArUrGrArUrGrGrCrUrArUrC*mC*mA*mG | |
| RNACS7679 | B2m1_ | mA*mC*mU*rCrArCrGrCrUrGrGrArUrArGrCrCrUrCrCrGrUrUrUrUrA | 17551 |
| | P9R19_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrUrUrCrUrGrGrCrCrUr | |
| | v1 | GrGrArUrGrArUrGrGrCrUrArU*mC*mC*mA | |
| RNACS7680 | B2m1_ | mA*mC*mU*rCrArCrGrCrUrGrGrArUrArGrCrCrUrCrCrGrUrUrUrUrA | 17552 |
| | P8R19_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrUrUrCrUrGrGrCrCrUr | |
| | v1 | GrGrArUrGrArUrGrGrCrUrA*mU*mC*mC | |
| RNACS7681 | B2m1_ | mA*mC*mU*rCrArCrGrCrUrGrGrArUrArGrCrCrUrCrCrGrUrUrUrUrA | 17553 |
| | P13R21_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrUrUrCrUrGrGrCr | |
| | v1 | CrUrGrGrArUrGrArUrGrGrCrUrArUrCrCrArG*mC*mG*mU | |
| RNACS7682 | B2m1_ | mA*mC*mU*rCrArCrGrCrUrGrGrArUrArGrCrCrUrCrCrGrUrUrUrUrA | 17554 |
| | P12R21_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrUrUrCrUrGrGrCr | |
| | v1 | CrUrGrGrArUrGrArUrGrGrCrUrArUrCrCrA*mG*mC*mG | |
| RNACS7683 | B2m1_ | mA*mC*mU*rCrArCrGrCrUrGrGrArUrArGrCrCrUrCrCrGrUrUrUrUrA | 17555 |
| | P11R21_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrUrUrCrUrGrGrCr | |
| | v1 | CrUrGrGrArUrGrArUrGrGrCrUrArUrCrC*mA*mG*mC | |
| RNACS7684 | B2m1_ | mA*mC*mU*rCrArCrGrCrUrGrGrArUrArGrCrCrUrCrCrGrUrUrUrUrA | 17556 |
| | P10R21_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrUrUrCrUrGrGr | |
| | v1 | CrCrUrGrGrArUrGrArUrGrGrCrUrArUrC*mC*mA*mG | |
| RNACS7685 | B2m1_ | mA*mC*mU*rCrArCrGrCrUrGrGrArUrArGrCrCrUrCrCrGrUrUrUrUrA | 17557 |
| | P9R21_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrUrUrCrUrGrGrCr | |
| | v1 | CrUrGrGrArUrGrArUrGrGrCrUrArU*mC*mC*mA | |
| RNACS7686 | B2m1_ | mA*mC*mU*rCrArCrGrCrUrGrGrArUrArGrCrCrUrCrCrGrUrUrUrUrA | 17558 |
| | P8R21_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrUrUrCrUrGrGrCr | |
| | v1 | CrUrGrGrArUrGrArUrGrGrCrUrA*mU*mC*mC | |
| RNACS7687 | B2m1_ | mA*mC*mU*rCrArCrGrCrUrGrGrArUrArGrCrCrUrCrCrGrUrUrUrUrA | 17559 |
| | P13R23_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrCrUrUrUrCrUrGr | |
| | v1 | GrCrCrUrGrGrArUrGrArUrGrGrCrUrArUrCrCrArG*mC*mG*mU | |
| RNACS7688 | B2m1_ | mA*mC*mU*rCrArCrGrCrUrGrGrArUrArGrCrCrUrCrCrGrUrUrUrUrA | 17560 |
| | P12R23_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrCrUrUrUrCrUrGr | |
| | v1 | GrCrCrUrGrGrArUrGrArUrGrGrCrUrArUrCrCrA*mG*mC*mG | |
| RNACS7689 | B2m1_ | mA*mC*mU*rCrArCrGrCrUrGrGrArUrArGrCrCrUrCrCrGrUrUrUrUrA | 17561 |
| | P11R23_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrCrUrUrUrCrUrGr | |
| | v1 | GrCrCrUrGrGrArUrGrArUrGrGrCrUrArUrCrC*mA*mG*mC | |
| RNACS7690 | B2m1_ | mA*mC*mU*rCrArCrGrCrUrGrGrArUrArGrCrCrUrCrCrGrUrUrUrUrA | 17562 |
| | P10R23_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrCrUrUrUrCrUrGr | |
| | v1 | GrCrCrUrGrGrArUrGrArUrGrGrCrUrArUrC*mC*mA*mG | |
| RNACS7691 | B2m1_ | mA*mC*mU*rCrArCrGrCrUrGrGrArUrArGrCrCrUrCrCrGrUrUrUrUrA | 17563 |
| | P9R23_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrCrUrUrUrCrUrGr | |
| | v1 | GrCrCrUrGrGrArUrGrArUrGrGrCrUrArU*mC*mC*mA | |
| RNACS7692 | B2m1_ | mA*mC*mU*rCrArCrGrCrUrGrGrArUrArGrCrCrUrCrCrGrUrUrUrUrA | 17564 |
| | P8R23_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrCrUrUrUrCrUrGr | |
| | v1 | GrCrCrUrGrGrArUrGrArUrGrGrCrUrA*mU*mC*mC | |
| RNACS7693 | B2m1_ | mA*mC*mU*rCrArCrGrCrUrGrGrArUrArGrCrCrUrCrCrGrUrUrUrUrA | 17565 |
| | P13R11_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrGrGrArUrArArCrGr | |
| | v2 | GrCrUrArUrCrCrArG*mC*mG*mU | |
| RNACS7694 | B2m1_ | mA*mC*mU*rCrArCrGrCrUrGrGrArUrArGrCrCrUrCrCrGrUrUrUrUrA | 17566 |
| | P12R11_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrGrGrArUrArArCrGr | |
| | v2 | GrCrUrArUrCrCrA*mG*mC*mG | |
| RNACS7695 | B2m1_ | mA*mC*mU*rCrArCrGrCrUrGrGrArUrArGrCrCrUrCrCrGrUrUrUrUrA | 17567 |
| | P11R11_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrGrGrArUrArArCrGr | |
| | v2 | GrCrUrArUrCrC*mA*mG*mC | |
| RNACS7696 | B2m1_ | mA*mC*mU*rCrArCrGrCrUrGrGrArUrArGrCrCrUrCrCrGrUrUrUrUrA | 17568 |
| | P10R11_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrGrGrArUrArArCrGr | |
| | v2 | GrCrUrArUrC*mC*mA*mG | |
| RNACS7697 | B2m1_ | mA*mC*mU*rCrArCrGrCrUrGrGrArUrArGrCrCrUrCrCrGrUrUrUrUrA | 17569 |
| | P9R11_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrGrGrArUrArArCrGr | |
| | v2 | GrCrUrArU*mC*mC*mA | |
| RNACS7698 | B2m1_ | mA*mC*mU*rCrArCrGrCrUrGrGrArUrArGrCrCrUrCrCrGrUrUrUrUrA | 17570 |
| | P8R11_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrGrGrArUrArArCrGr | |
| | v2 | GrCrUrA*mU*mC*mC | |
| RNACS7699 | B2m1_ | mA*mC*mU*rCrArCrGrCrUrGrGrArUrArGrCrCrUrCrCrGrUrUrUrUrA | 17571 |
| | P13R13_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrGrGrArUrArAr | |
| | v2 | CrGrGrCrUrArUrCrCrArG*mC*mG*mU | |
| RNACS7700 | B2m1_ | mA*mC*mU*rCrArCrGrCrUrGrGrArUrArGrCrCrUrCrCrGrUrUrUrUrA | 17572 |
| | P12R13_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrGrGrArUrArAr | |
| | v2 | CrGrGrCrUrArUrCrCrA*mG*mC*mG | |
| RNACS7701 | B2m1_ | mA*mC*mU*rCrArCrGrCrUrGrGrArUrArGrCrCrUrCrCrGrUrUrUrUrA | 17573 |
| | P11R13_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrGrGrArUrArAr | |
| | v2 | CrGrGrCrUrArUrCrC*mA*mG*mC | |
| RNACS7702 | B2m1_ | mA*mC*mU*rCrArCrGrCrUrGrGrArUrArGrCrCrUrCrCrGrUrUrUrUrA | 17574 |
| | P10R13_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrGrGrArUrArAr | |
| | v2 | CrGrGrCrUrArUrC*mC*mA*mG | |
| RNACS7703 | B2m1_ | mA*mC*mU*rCrArCrGrCrUrGrGrArUrArGrCrCrUrCrCrGrUrUrUrUrA | 17575 |
| | P9R13_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrGrGrArUrArAr | |
| | v2 | CrGrGrCrUrArU*mC*mC*mA | |
| RNACS7704 | B2m1_ | mA*mC*mU*rCrArCrGrCrUrGrGrArUrArGrCrCrUrCrCrGrUrUrUrUrA | 17576 |
| | P8R13_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrGrGrArUrArAr | |
| | v2 | CrGrGrCrUrA*mU*mC*mC | |
| RNACS7705 | B2m1_ | mA*mC*mU*rCrArCrGrCrUrGrGrArUrArGrCrCrUrCrCrGrUrUrUrUrA | 17577 |
| | P13R15_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrGrGrCrCrUrGrGrArUr | |
| | v2 | ArArCrGrGrCrUrArUrCrCrArG*mC*mG*mU | |
| RNACS7706 | B2m1_ | mA*mC*mU*rCrArCrGrCrUrGrGrArUrArGrCrCrUrCrCrGrUrUrUrUrA | 17578 |
| | P12R15_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrGrGrCrCrUrGrGrArUr | |
| | v2 | ArArCrGrGrCrUrArUrCrCrA*mG*mC*mG | |
| RNACS7707 | B2m1_ | mA*mC*mU*rCrArCrGrCrUrGrGrArUrArGrCrCrUrCrCrGrUrUrUrUrA | 17579 |
| | P11R15_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrGrGrCrCrUrGrGrArUr | |
| | v2 | ArArCrGrGrCrUrArUrCrC*mA*mG*mC | |
| RNACS7708 | B2m1_ | mA*mC*mU*rCrArCrGrCrUrGrGrArUrArGrCrCrUrCrCrGrUrUrUrUrA | 17580 |
| | P10R15_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrGrGrCrCrUrGrGrArUr | |
| | v2 | ArArCrGrGrCrUrArUrC*mC*mA*mG | |
| RNACS7709 | B2m1_ | mA*mC*mU*rCrArCrGrCrUrGrGrArUrArGrCrCrUrCrCrGrUrUrUrUrA | 17581 |
| | P9R15_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrGrGrCrCrUrGrGrArUr | |
| | v2 | ArArCrGrGrCrUrArU*mC*mC*mA | |
| RNACS7710 | B2m1_ | mA*mC*mU*rCrArCrGrCrUrGrGrArUrArGrCrCrUrCrCrGrUrUrUrUrA | 17582 |
| | P8R15_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrGrGrCrCrUrGrGrArUr | |
| | v2 | ArArCrGrGrCrUrA*mU*mC*mC | |
| RNACS7711 | B2m1_ | mA*mC*mU*rCrArCrGrCrUrGrGrArUrArGrCrCrUrCrCrGrUrUrUrUrA | 17583 |
| | P13R17_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrUrUrCrUrGrGrCrCrUrGrGr | |
| | v2 | ArUrArArCrGrGrCrUrArUrCrCrArG*mC*mG*mU | |
| RNACS7712 | B2m1_ | mA*mC*mU*rCrArCrGrCrUrGrGrArUrArGrCrCrUrCrCrGrUrUrUrUrA | 17584 |
| | P12R17_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrUrUrCrUrGrGrCrCrUrGrGr | |
| | v2 | ArUrArArCrGrGrCrUrArUrCrCrA*mG*mC*mG | |
| RNACS7713 | B2m1_ | mA*mC*mU*rCrArCrGrCrUrGrGrArUrArGrCrCrUrCrCrGrUrUrUrUrA | 17585 |
| | P11R17_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrUrUrCrUrGrGrCrCrUrGrGr | |
| | v2 | ArUrArArCrGrGrCrUrArUrCrC*mA*mG*mC | |
| RNACS7714 | B2m1_ | mA*mC*mU*rCrArCrGrCrUrGrGrArUrArGrCrCrUrCrCrGrUrUrUrUrA | 17586 |
| | P10R17_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrUrUrCrUrGrGrCrCrUrGrGr | |
| | v2 | ArUrArArCrGrGrCrUrArUrC*mC*mA*mG | |
| RNACS7715 | B2m1_ | mA*mC*mU*rCrArCrGrCrUrGrGrArUrArGrCrCrUrCrCrGrUrUrUrUrA | 17587 |
| | P9R17_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrUrUrCrUrGrGrCrCrUrGrGr | |
| | v2 | ArUrArArCrGrGrCrUrArU*mC*mC*mA | |
| RNACS7716 | B2m1_ | mA*mC*mU*rCrArCrGrCrUrGrGrArUrArGrCrCrUrCrCrGrUrUrUrUrA | 17588 |
| | P8R17_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrUrUrCrUrGrGrCrCrUrGrGr | |
| | v2 | ArUrArArCrGrGrCrUrA*mU*mC*mC | |
| RNACS7717 | B2m1_ | mA*mC*mU*rCrArCrGrCrUrGrGrArUrArGrCrCrUrCrCrGrUrUrUrUrA | 17589 |
| | P13R19_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrUrUrCrUrGrGrCrCrUr | |
| | v2 | GrGrArUrArArCrGrGrCrUrArUrCrCrArG*mC*mG*mU | |
| RNACS7718 | B2m1_ | mA*mC*mU*rCrArCrGrCrUrGrGrArUrArGrCrCrUrCrCrGrUrUrUrUrA | 17590 |
| | P12R19_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrUrUrCrUrGrGrCrCrUr | |
| | v2 | GrGrArUrArArCrGrGrCrUrArUrCrCrA*mG*mC*mG | |
| RNACS7719 | B2m1_ | mA*mC*mU*rCrArCrGrCrUrGrGrArUrArGrCrCrUrCrCrGrUrUrUrUrA | 17591 |
| | P11R19_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrUrUrCrUrGrGrCrCrUr | |
| | v2 | GrGrArUrArArCrGrGrCrUrArUrCrC*mA*mG*mC | |
| RNACS7720 | B2m1_ | mA*mC*mU*rCrArCrGrCrUrGrGrArUrArGrCrCrUrCrCrGrUrUrUrUrA | 17592 |
| | P10R19_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrUrUrCrUrGrGrCrCrUr | |
| | v2 | GrGrArUrArArCrGrGrCrUrArUrC*mC*mA*mG | |
| RNACS7721 | B2m1_ | mA*mC*mU*rCrArCrGrCrUrGrGrArUrArGrCrCrUrCrCrGrUrUrUrUrA | 17593 |
| | P9R19_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrUrUrCrUrGrGrCrCrUr | |
| | v2 | GrGrArUrArArCrGrGrCrUrArU*mC*mC*mA | |
| RNACS7722 | B2m1_ | mA*mC*mU*rCrArCrGrCrUrGrGrArUrArGrCrCrUrCrCrGrUrUrUrUrA | 17594 |
| | P8R19_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrUrUrCrUrGrGrCrCrUr | |
| | v2 | GrGrArUrArArCrGrGrCrUrA*mU*mC*mC | |
| RNACS7723 | B2m1_ | mA*mC*mU*rCrArCrGrCrUrGrGrArUrArGrCrCrUrCrCrGrUrUrUrUrA | 17595 |
| | P13R21_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrUrUrCrUrGrGrCr | |
| | v2 | CrUrGrGrArUrArArCrGrGrCrUrArUrCrCrArG*mC*mG*mU | |
| RNACS7724 | B2m1_ | mA*mC*mU*rCrArCrGrCrUrGrGrArUrArGrCrCrUrCrCrGrUrUrUrUrA | 17596 |
| | P12R21_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrUrUrCrUrGrGrCr | |
| | v2 | CrUrGrGrArUrArArCrGrGrCrUrArUrCrCrA*mG*mC*mG | |
| RNACS7725 | B2m1_ | mA*mC*mU*rCrArCrGrCrUrGrGrArUrArGrCrCrUrCrCrGrUrUrUrUrA | 17597 |
| | P11R21_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrUrUrCrUrGrGrCr | |
| | v2 | CrUrGrGrArUrArArCrGrGrCrUrArUrCrC*mA*mG*mC | |
| RNACS7726 | B2m1_ | mA*mC*mU*rCrArCrGrCrUrGrGrArUrArGrCrCrUrCrCrGrUrUrUrUrA | 17598 |
| | P10R21_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrUrUrCrUrGrGrCr | |
| | v2 | CrUrGrGrArUrArArCrGrGrCrUrArUrC*mC*mA*mG | |
| RNACS7727 | B2m1_ | mA*mC*mU*rCrArCrGrCrUrGrGrArUrArGrCrCrUrCrCrGrUrUrUrUrA | 17599 |
| | P9R21_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrUrUrCrUrGrGr | |
| | v2 | CrCrUrGrGrArUrArArCrGrGrCrUrArU*mC*mC*mA | |
| RNACS7728 | B2m1_ | mA*mC*mU*rCrArCrGrCrUrGrGrArUrArGrCrCrUrCrCrGrUrUrUrUrA | 17600 |
| | P8R21_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrUrUrCrUrGrGrCr | |
| | v2 | CrUrGrGrArUrArArCrGrGrCrUrA*mU*mC*mC | |
| RNACS7729 | B2m1_ | mA*mC*mU*rCrArCrGrCrUrGrGrArUrArGrCrCrUrCrCrGrUrUrUrUrA | 17601 |
| | P13R23_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrCrUrUrUrCrUrGr | |
| | v2 | GrCrCrUrGrGrArUrArArCrGrGrCrUrArUrCrCrArG*mC*mG*mU | |
| RNACS7730 | B2m1_ | mA*mC*mU*rCrArCrGrCrUrGrGrArUrArGrCrCrUrCrCrGrUrUrUrUrA | 17602 |
| | P12R23_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrCrUrUrUrCrUrGr | |
| | v2 | GrCrCrUrGrGrArUrArArCrGrGrCrUrArUrCrCrA*mG*mC*mG | |
| RNACS7731 | B2m1_ | mA*mC*mU*rCrArCrGrCrUrGrGrArUrArGrCrCrUrCrCrGrUrUrUrUrA | 17603 |
| | P11R23_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrCrUrUrUrCrUrGr | |
| | v2 | GrCrCrUrGrGrArUrArArCrGrGrCrUrArUrCrC*mA*mG*mC | |
| RNACS7732 | B2m1_ | mA*mC*mU*rCrArCrGrCrUrGrGrArUrArGrCrCrUrCrCrGrUrUrUrUrA | 17604 |
| | P10R23_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrCrUrUrUrCrUrGr | |
| | v2 | GrCrCrUrGrGrArUrArArCrGrGrCrUrArUrC*mC*mA*mG | |
| RNACS7733 | B2m1_ | mA*mC*mU*rCrArCrGrCrUrGrGrArUrArGrCrCrUrCrCrGrUrUrUrUrA | 17605 |
| | P9R23_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrCrUrUrUrCrUrGr | |
| | v2 | GrCrCrUrGrGrArUrArArCrGrGrCrUrArU*mC*mC*mA | |
| RNACS7734 | B2m1_ | mA*mC*mU*rCrArCrGrCrUrGrGrArUrArGrCrCrUrCrCrGrUrUrUrUrA | 17606 |
| | P8R23_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrCrUrUrUrCrUrGr | |
| | v2 | GrCrCrUrGrGrArUrArArCrGrGrCrUrA*mU*mC*mC | |
| RNACS7735 | B2m1_ | mA*mC*mU*rCrArCrGrCrUrGrGrArUrArGrCrCrUrCrCrGrUrUrUrUrA | 17607 |
| | P13R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrGrGrArUrGrArUrGr | |
| | v1 | GrGrCrUrArUrCrCrArG*mC*mG*mU | |
| RNACS7736 | B2m1_ | mA*mC*mU*rCrArCrGrCrUrGrGrArUrArGrCrCrUrCrCrGrUrUrUrUrA | 17608 |
| | P12R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrGrGrArUrGrArUrGr | |
| | v1 | GrGrCrUrArUrCrCrA*mG*mC*mG | |
| RNACS7737 | B2m1_ | mA*mC*mU*rCrArCrGrCrUrGrGrArUrArGrCrCrUrCrCrGrUrUrUrUrA | 17609 |
| | P11R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrGrGrArUrGrArUrGr | |
| | v1 | GrGrCrUrArUrCrC*mA*mG*mC | |
| RNACS7738 | B2m1_ | mA*mC*mU*rCrArCrGrCrUrGrGrArUrArGrCrCrUrCrCrGrUrUrUrUrA | 17610 |
| | P10R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrGrGrArUrGrArUrGr | |
| | v1 | GrGrCrUrArUrC*mC*mA*mG | |
| RNACS7739 | B2m1_ | mA*mC*mU*rCrArCrGrCrUrGrGrArUrArGrCrCrUrCrCrGrUrUrUrUrA | 17611 |
| | P9R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrGrGrArUrGrArUrGr | |
| | v1 | GrGrCrUrArU*mC*mC*mA | |
| RNACS7740 | B2m1_ | mA*mC*mU*rCrArCrGrCrUrGrGrArUrArGrCrCrUrCrCrGrUrUrUrUrA | 17612 |
| | P8R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrGrGrArUrGrArUrGr | |
| | v1 | GrGrCrUrA*mU*mC*mC | |
| RNACS7741 | B2m1_ | mA*mC*mU*rCrArCrGrCrUrGrGrArUrArGrCrCrUrCrCrGrUrUrUrUrA | 17613 |
| | P13R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrGrGrArUrGrAr | |
| | v1 | UrGrGrGrCrUrArUrCrCrArG*mC*mG*mU | |
| RNACS7742 | B2m1_ | mA*mC*mU*rCrArCrGrCrUrGrGrArUrArGrCrCrUrCrCrGrUrUrUrUrA | 17614 |
| | P12R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrGrGrArUrGrAr | |
| | v1 | UrGrGrGrCrUrArUrCrCrA*mG*mC*mG | |
| RNACS7743 | B2m1_ | mA*mC*mU*rCrArCrGrCrUrGrGrArUrArGrCrCrUrCrCrGrUrUrUrUrA | 17615 |
| | P11R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrGrGrArUrGrAr | |
| | v1 | UrGrGrGrCrUrArUrCrC*mA*mG*mC | |
| RNACS7744 | B2m1_ | mA*mC*mU*rCrArCrGrCrUrGrGrArUrArGrCrCrUrCrCrGrUrUrUrUrA | 17616 |
| | P10R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrGrGrArUrGrAr | |
| | v1 | UrGrGrGrCrUrArUrC*mC*mA*mG | |
| RNACS7745 | B2m1_ | mA*mC*mU*rCrArCrGrCrUrGrGrArUrArGrCrCrUrCrCrGrUrUrUrUrA | 17617 |
| | P9R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrGrGrArUrGrAr | |
| | v1 | UrGrGrGrCrUrArU*mC*mC*mA | |
| RNACS7746 | B2m1_ | mA*mC*mU*rCrArCrGrCrUrGrGrArUrArGrCrCrUrCrCrGrUrUrUrUrA | 17618 |
| | P8R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrGrGrArUrGrAr | |
| | v1 | UrGrGrGrCrUrA*mU*mC*mC | |
| RNACS7747 | B2m1_ | mA*mC*mU*rCrArCrGrCrUrGrGrArUrArGrCrCrUrCrCrGrUrUrUrUrA | 17619 |
| | P13R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrGrGrCrCrUrGrGrArUr | |
| | v1 | GrArUrGrGrGrCrUrArUrCrCrArG*mC*mG*mU | |
| RNACS7748 | B2m1_ | mA*mC*mU*rCrArCrGrCrUrGrGrArUrArGrCrCrUrCrCrGrUrUrUrUrA | 17620 |
| | P12R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrGrGrCrCrUrGrGrArUr | |
| | v1 | GrArUrGrGrGrCrUrArUrCrCrA*mG*mC*mG | |
| RNACS7749 | B2m1_ | mA*mC*mU*rCrArCrGrCrUrGrGrArUrArGrCrCrUrCrCrGrUrUrUrUrA | 17621 |
| | P11R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrGrGrCrCrUrGrGrArUr | |
| | v1 | GrArUrGrGrGrCrUrArUrCrC*mA*mG*mC | |
| RNACS7750 | B2m1_ | mA*mC*mU*rCrArCrGrCrUrGrGrArUrArGrCrCrUrCrCrGrUrUrUrUrA | 17622 |
| | P10R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrGrGrCrCrUrGrGrArUr | |
| | v1 | GrArUrGrGrGrCrUrArUrC*mC*mA*mG | |
| RNACS7751 | B2m1_ | mA*mC*mU*rCrArCrGrCrUrGrGrArUrArGrCrCrUrCrCrGrUrUrUrUrA | 17623 |
| | P9R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrGrGrCrCrUrGrGrArUr | |
| | v1 | GrArUrGrGrGrCrUrArU*mC*mC*mA | |
| RNACS7752 | B2m1_ | mA*mC*mU*rCrArCrGrCrUrGrGrArUrArGrCrCrUrCrCrGrUrUrUrUrA | 17624 |
| | P8R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrGrGrCrCrUrGrGrArUr | |
| | v1 | GrArUrGrGrGrCrUrA*mU*mC*mC | |
| RNACS7753 | B2m1_ | mA*mC*mU*rCrArCrGrCrUrGrGrArUrArGrCrCrUrCrCrGrUrUrUrUrA | 17625 |
| | P13R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrUrUrCrUrGrGrCrCrUrGrGr | |
| | v1 | ArUrGrArUrGrGrGrCrUrArUrCrCrArG*mC*mG*mU | |
| RNACS7754 | B2m1_ | mA*mC*mU*rCrArCrGrCrUrGrGrArUrArGrCrCrUrCrCrGrUrUrUrUrA | 17626 |
| | P12R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrUrUrCrUrGrGrCrCrUrGrGr | |
| | v1 | ArUrGrArUrGrGrGrCrUrArUrCrCrA*mG*mC*mG | |
| RNACS7755 | B2m1_ | mA*mC*mU*rCrArCrGrCrUrGrGrArUrArGrCrCrUrCrCrGrUrUrUrUrA | 17627 |
| | P11R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrUrUrCrUrGrGrCrCrUrGrGr | |
| | v1 | ArUrGrArUrGrGrGrCrUrArUrCrC*mA*mG*mC | |
| RNACS7756 | B2m1_ | mA*mC*mU*rCrArCrGrCrUrGrGrArUrArGrCrCrUrCrCrGrUrUrUrUrA | 17628 |
| | P10R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrUrUrCrUrGrGrCrCrUrGrGr | |
| | v1 | ArUrGrArUrGrGrGrCrUrArUrC*mC*mA*mG | |
| RNACS7757 | B2m1_ | mA*mC*mU*rCrArCrGrCrUrGrGrArUrArGrCrCrUrCrCrGrUrUrUrUrA | 17629 |
| | P9R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrUrUrCrUrGrGrCrCrUrGrGr | |
| | v1 | ArUrGrArUrGrGrGrCrUrArU*mC*mC*mA | |
| RNACS7758 | B2m1_ | mA*mC*mU*rCrArCrGrCrUrGrGrArUrArGrCrCrUrCrCrGrUrUrUrUrA | 17630 |
| | P8R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrUrUrCrUrGrGrCrCrUrGrGr | |
| | v1 | ArUrGrArUrGrGrGrCrUrA*mU*mC*mC | |
| RNACS7759 | B2m1_ | mA*mC*mU*rCrArCrGrCrUrGrGrArUrArGrCrCrUrCrCrGrUrUrUrUrA | 17631 |
| | P13R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrUrUrCrUrGrGrCrCrUr | |
| | v1 | GrGrArUrGrArUrGrGrGrCrUrArUrCrCrArG*mC*mG*mU | |
| RNACS7760 | B2m1_ | mA*mC*mU*rCrArCrGrCrUrGrGrArUrArGrCrCrUrCrCrGrUrUrUrUrA | 17632 |
| | P12R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrUrUrCrUrGrGrCrCrUr | |
| | v1 | GrGrArUrGrArUrGrGrGrCrUrArUrCrCrA*mG*mC*mG | |
| RNACS7761 | B2m1_ | mA*mC*mU*rCrArCrGrCrUrGrGrArUrArGrCrCrUrCrCrGrUrUrUrUrA | 17633 |
| | P11R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrUrUrCrUrGrGrCrCrUr | |
| | v1 | GrGrArUrGrArUrGrGrGrCrUrArUrCrC*mA*mG*mC | |
| RNACS7762 | B2m1_ | mA*mC*mU*rCrArCrGrCrUrGrGrArUrArGrCrCrUrCrCrGrUrUrUrUrA | 17634 |
| | P10R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrUrUrCrUrGrGrCrCrUr | |
| | v1 | GrGrArUrGrArUrGrGrGrCrUrArUrC*mC*mA*mG | |
| RNACS7763 | B2m1_ | mA*mC*mU*rCrArCrGrCrUrGrGrArUrArGrCrCrUrCrCrGrUrUrUrUrA | 17635 |
| | P9R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrUrUrCrUrGrGrCrCrUr | |
| | v1 | GrGrArUrGrArUrGrGrGrCrUrArU*mC*mC*mA | |
| RNACS7764 | B2m1_ | mA*mC*mU*rCrArCrGrCrUrGrGrArUrArGrCrCrUrCrCrGrUrUrUrUrA | 17636 |
| | P8R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrUrUrCrUrGrGrCrCrUr | |
| | v1 | GrGrArUrGrArUrGrGrGrCrUrA*mU*mC*mC | |
| RNACS7765 | B2m1_ | mA*mC*mU*rCrArCrGrCrUrGrGrArUrArGrCrCrUrCrCrGrUrUrUrUrA | 17637 |
| | P13R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrUrUrCrUrGrGrCr | |
| | v1 | CrUrGrGrArUrGrArUrGrGrGrCrUrArUrCrCrArG*mC*mG*mU | |
| RNACS7766 | B2m1_ | mA*mC*mU*rCrArCrGrCrUrGrGrArUrArGrCrCrUrCrCrGrUrUrUrUrA | 17638 |
| | P12R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrUrUrCrUrGrGrCr | |
| | v1 | CrUrGrGrArUrGrArUrGrGrGrCrUrArUrCrCrA*mG*mC*mG | |
| RNACS7767 | B2m1_ | mA*mC*mU*rCrArCrGrCrUrGrGrArUrArGrCrCrUrCrCrGrUrUrUrUrA | 17639 |
| | P11R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrUrUrCrUrGrGrCr | |
| | v1 | CrUrGrGrArUrGrArUrGrGrGrCrUrArUrCrC*mA*mG*mC | |
| RNACS7768 | B2m1_ | mA*mC*mU*rCrArCrGrCrUrGrGrArUrArGrCrCrUrCrCrGrUrUrUrUrA | 17640 |
| | P10R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrUrUrCrUrGrGrCr | |
| | v1 | CrUrGrGrArUrGrArUrGrGrGrCrUrArUrC*mC*mA*mG | |
| RNACS7769 | B2m1_ | mA*mC*mU*rCrArCrGrCrUrGrGrArUrArGrCrCrUrCrCrGrUrUrUrUrA | 17641 |
| | P9R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrUrUrCrUrGrGrCr | |
| | v1 | CrUrGrGrArUrGrArUrGrGrGrCrUrArUmC*mC*mA | |
| RNACS7770 | B2m1_ | mA*mC*mU*rCrArCrGrCrUrGrGrArUrArGrCrCrUrCrCrGrUrUrUrUrA | 17642 |
| | P8R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrUrUrCrUrGrGrCr | |
| | v1 | CrUrGrGrArUrGrArUrGrGrGrCrUrA*mU*mC*mC | |
| RNACS7771 | B2m1_ | mA*mC*mU*rCrArCrGrCrUrGrGrArUrArGrCrCrUrCrCrGrUrUrUrUrA | 17643 |
| | P13R24_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrCrUrUrUrCrUrGr | |
| | v1 | GrCrCrUrGrGrArUrGrArUrGrGrGrCrUrArUrCrCrArG*mC*mG*mU | |
| RNACS7772 | B2m1_ | mA*mC*mU*rCrArCrGrCrUrGrGrArUrArGrCrCrUrCrCrGrUrUrUrUrA | 17644 |
| | P12R24_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrCrUrUrUrCrUrGr | |
| | v1 | GrCrCrUrGrGrArUrGrArUrGrGrGrCrUrArUrCrCrA*mG*mC*mG | |
| RNACS7773 | B2m1_ | mA*mC*mU*rCrArCrGrCrUrGrGrArUrArGrCrCrUrCrCrGrUrUrUrUrA | 17645 |
| | P11R24_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrCrUrUrUrCrUrGr | |
| | v1 | GrCrCrUrGrGrArUrGrArUrGrGrGrCrUrArUrCrC*mA*mG*mC | |
| RNACS7774 | B2m1_ | mA*mC*mU*rCrArCrGrCrUrGrGrArUrArGrCrCrUrCrCrGrUrUrUrUrA | 17646 |
| | P10R24_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrCrUrUrUrCrUrGr | |
| | v1 | GrCrCrUrGrGrArUrGrArUrGrGrGrCrUrArUrC*mC*mA*mG | |
| RNACS7775 | B2m1_ | mA*mC*mU*rCrArCrGrCrUrGrGrArUrArGrCrCrUrCrCrGrUrUrUrUrA | 17647 |
| | P9R24_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrCrUrUrUrCrUrGr | |
| | v1 | GrCrCrUrGrGrArUrGrArUrGrGrGrCrUrArU*mC*mC*mA | |
| RNACS7776 | B2m1_ | mA*mC*mU*rCrArCrGrCrUrGrGrArUrArGrCrCrUrCrCrGrUrUrUrUrA | 17648 |
| | P8R24_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrCrUrUrUrCrUrGr | |
| | v1 | GrCrCrUrGrGrArUrGrArUrGrGrGrCrUrA*mU*mC*mC | |
| RNACS7777 | B2m1_ | mA*mC*mU*rCrArCrGrCrUrGrGrArUrArGrCrCrUrCrCrGrUrUrUrUrA | 17649 |
| | P13R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrGrGrArUrArArCrCr | |
| | v2 | GrGrCrUrArUrCrCrArG*mC*mG*mU | |
| RNACS7778 | B2m1_ | mA*mC*mU*rCrArCrGrCrUrGrGrArUrArGrCrCrUrCrCrGrUrUrUrUrA | 17650 |
| | P12R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrGrGrArUrArArCrCr | |
| | v2 | GrGrCrUrArUrCrCrA*mG*mC*mG | |
| RNACS7779 | B2m1_ | mA*mC*mU*rCrArCrGrCrUrGrGrArUrArGrCrCrUrCrCrGrUrUrUrUrA | 17651 |
| | P11R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrGrGrArUrArArCrCr | |
| | v2 | GrGrCrUrArUrCrC*mA*mG*mC | |
| RNACS7780 | B2m1_ | mA*mC*mU*rCrArCrGrCrUrGrGrArUrArGrCrCrUrCrCrGrUrUrUrUrA | 17652 |
| | P10R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrGrGrArUrArArCr | |
| | v2 | CrGrGrCrUrArUrC*mC*mA*mG | |
| RNACS7781 | B2m1_ | mA*mC*mU*rCrArCrGrCrUrGrGrArUrArGrCrCrUrCrCrGrUrUrUrUrA | 17653 |
| | P9R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrGrGrArUrArArCrCr | |
| | v2 | GrGrCrUrArU*mC*mC*mA | |
| RNACS7782 | B2m1_ | mA*mC*mU*rCrArCrGrCrUrGrGrArUrArGrCrCrUrCrCrGrUrUrUrUrA | 17654 |
| | P8R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrGrGrArUrArArCrCr | |
| | v2 | GrGrCrUrA*mU*mC*mC | |
| RNACS7783 | B2m1_ | mA*mC*mU*rCrArCrGrCrUrGrGrArUrArGrCrCrUrCrCrGrUrUrUrUrA | 17655 |
| | P13R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrGrGrArUrAr | |
| | v2 | ArCrCrGrGrCrUrArUrCrCrArG*mC*mG*mU | |
| RNACS7784 | B2m1_ | mA*mC*mU*rCrArCrGrCrUrGrGrArUrArGrCrCrUrCrCrGrUrUrUrUrA | 17656 |
| | P12R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrGrGrArUrArAr | |
| | v2 | CrCrGrGrCrUrArUrCrCrA*mG*mC*mG | |
| RNACS7785 | B2m1_ | mA*mC*mU*rCrArCrGrCrUrGrGrArUrArGrCrCrUrCrCrGrUrUrUrUrA | 17657 |
| | P11R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrGrGrArUrArAr | |
| | v2 | CrCrGrGrCrUrArUrCrC*mA*mG*mC | |
| RNACS7786 | B2m1_ | mA*mC*mU*rCrArCrGrCrUrGrGrArUrArGrCrCrUrCrCrGrUrUrUrUrA | 17658 |
| | P10R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrGrGrArUrArAr | |
| | v2 | CrCrGrGrCrUrArUrC*mC*mA*mG | |
| RNACS7787 | B2m1_ | mA*mC*mU*rCrArCrGrCrUrGrGrArUrArGrCrCrUrCrCrGrUrUrUrUrA | 17659 |
| | P9R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrGrGrArUrArAr | |
| | v2 | CrCrGrGrCrUrArU*mC*mC*mA | |
| RNACS7788 | B2m1_ | mA*mC*mU*rCrArCrGrCrUrGrGrArUrArGrCrCrUrCrCrGrUrUrUrUrA | 17660 |
| | P8R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrGrGrArUrArAr | |
| | v2 | CrCrGrGrCrUrA*mU*mC*mC | |
| RNACS7789 | B2m1_ | mA*mC*mU*rCrArCrGrCrUrGrGrArUrArGrCrCrUrCrCrGrUrUrUrUrA | 17661 |
| | P13R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrGrGrCrCrUrGrGrArUr | |
| | v2 | ArArCrCrGrGrCrUrArUrCrCrArG*mC*mG*mU | |
| RNACS7790 | B2m1_ | mA*mC*mU*rCrArCrGrCrUrGrGrArUrArGrCrCrUrCrCrGrUrUrUrUrA | 17662 |
| | P12R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrGrGrCrCrUrGrGrArUr | |
| | v2 | ArArCrCrGrGrCrUrArUrCrCrA*mG*mC*mG | |
| RNACS7791 | B2m1_ | mA*mC*mU*rCrArCrGrCrUrGrGrArUrArGrCrCrUrCrCrGrUrUrUrUrA | 17663 |
| | P11R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrGrGrCrCrUrGrGrArUr | |
| | v2 | ArArCrCrGrGrCrUrArUrCrC*mA*mG*mC | |
| RNACS7792 | B2m1_ | mA*mC*mU*rCrArCrGrCrUrGrGrArUrArGrCrCrUrCrCrGrUrUrUrUrA | 17664 |
| | P10R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrGrGrCrCrUrGrGrAr | |
| | v2 | UrArArCrCrGrGrCrUrArUrC*mC*mA*mG | |
| RNACS7793 | B2m1_ | mA*mC*mU*rCrArCrGrCrUrGrGrArUrArGrCrCrUrCrCrGrUrUrUrUrA | 17665 |
| | P9R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrGrGrCrCrUrGrGrAr | |
| | v2 | UrArArCrCrGrGrCrUrArU*mC*mC*mA | |
| RNACS7794 | B2m1_ | mA*mC*mU*rCrArCrGrCrUrGrGrArUrArGrCrCrUrCrCrGrUrUrUrUrA | 17666 |
| | P8R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrGrGrCrCrUrGrGrArUr | |
| | v2 | ArArCrCrGrGrCrUrA*mU*mC*mC | |
| RNACS7795 | B2m1_ | mA*mC*mU*rCrArCrGrCrUrGrGrArUrArGrCrCrUrCrCrGrUrUrUrUrA | 17667 |
| | P13R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrUrUrCrUrGrGrCrCrUrGrGr | |
| | v2 | ArUrArArCrCrGrGrCrUrArUrCrCrArG*mC*mG*mU | |
| RNACS7796 | B2m1_ | mA*mC*mU*rCrArCrGrCrUrGrGrArUrArGrCrCrUrCrCrGrUrUrUrUrA | 17668 |
| | P12R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrUrUrCrUrGrGrCrCrUrGrGr | |
| | v2 | ArUrArArCrCrGrGrCrUrArUrCrCrA*mG*mC*mG | |
| RNACS7797 | B2m1_ | mA*mC*mU*rCrArCrGrCrUrGrGrArUrArGrCrCrUrCrCrGrUrUrUrUrA | 17669 |
| | P11R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrUrUrCrUrGrGrCrCrUrGrGr | |
| | v2 | ArUrArArCrCrGrGrCrUrArUrCrC*mA*mG*mC | |
| RNACS7798 | B2m1_ | mA*mC*mU*rCrArCrGrCrUrGrGrArUrArGrCrCrUrCrCrGrUrUrUrUrA | 17670 |
| | P10R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrUrUrCrUrGrGrCrCrUrGrGr | |
| | v2 | ArUrArArCrCrGrGrCrUrArUrC*mC*mA*mG | |
| RNACS7799 | B2m1_ | mA*mC*mU*rCrArCrGrCrUrGrGrArUrArGrCrCrUrCrCrGrUrUrUrUrA | 17671 |
| | P9R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrUrUrCrUrGrGrCrCrUrGrGr | |
| | v2 | ArUrArArCrCrGrGrCrUrArU*mC*mC*mA | |
| RNACS7800 | B2m1_ | mA*mC*mU*rCrArCrGrCrUrGrGrArUrArGrCrCrUrCrCrGrUrUrUrUrA | 17672 |
| | P8R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrUrUrCrUrGrGrCrCrUrGrGr | |
| | v2 | ArUrArArCrCrGrGrCrUrA*mU*mC*mC | |
| RNACS7801 | B2m1_ | mA*mC*mU*rCrArCrGrCrUrGrGrArUrArGrCrCrUrCrCrGrUrUrUrUrA | 17673 |
| | P13R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrUrUrCrUrGrGrCrCrUr | |
| | v2 | GrGrArUrArArCrCrGrGrCrUrArUrCrCrArG*mC*mG*mU | |
| RNACS7802 | B2m1_ | mA*mC*mU*rCrArCrGrCrUrGrGrArUrArGrCrCrUrCrCrGrUrUrUrUrA | 17674 |
| | P12R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrUrUrCrUrGrGrCrCrUr | |
| | v2 | GrGrArUrArArCrCrGrGrCrUrArUrCrCrA*mG*mC*mG | |
| RNACS7803 | B2m1_ | mA*mC*mU*rCrArCrGrCrUrGrGrArUrArGrCrCrUrCrCrGrUrUrUrUrA | 17675 |
| | P11R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrUrUrCrUrGrGrCrCr | |
| | v2 | UrGrGrArUrArArCrCrGrGrCrUrArUrCrC*mA*mG*mC | |
| RNACS7804 | B2m1_ | mA*mC*mU*rCrArCrGrCrUrGrGrArUrArGrCrCrUrCrCrGrUrUrUrUrA | 17676 |
| | P10R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrUrUrCrUrGrGrCrCrUr | |
| | v2 | GrGrArUrArArCrCrGrGrCrUrArUrC*mC*mA*mG | |
| RNACS7805 | B2m1_ | mA*mC*mU*rCrArCrGrCrUrGrGrArUrArGrCrCrUrCrCrGrUrUrUrUrA | 17677 |
| | P9R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrUrUrCrUrGrGrCrCrUr | |
| | v2 | GrGrArUrArArCrCrGrGrCrUrArU*mC*mC*mA | |
| RNACS7806 | B2m1_ | mA*mC*mU*rCrArCrGrCrUrGrGrArUrArGrCrCrUrCrCrGrUrUrUrUrA | 17678 |
| | P8R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrUrUrCrUrGrGrCrCrUr | |
| | v2 | GrGrArUrArArCrCrGrGrCrUrA*mU*mC*mC | |
| RNACS7807 | B2m1_ | mA*mC*mU*rCrArCrGrCrUrGrGrArUrArGrCrCrUrCrCrGrUrUrUrUrA | 17679 |
| | P13R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrUrUrCrUrGrGrCr | |
| | v2 | CrUrGrGrArUrArArCrCrGrGrCrUrArUrCrCrArG*mC*mG*mU | |
| RNACS7808 | B2m1_ | mA*mC*mU*rCrArCrGrCrUrGrGrArUrArGrCrCrUrCrCrGrUrUrUrUrA | 17680 |
| | P12R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrUrUrCrUrGrGrCr | |
| | v2 | CrUrGrGrArUrArArCrCrGrGrCrUrArUrCrCrA*mG*mC*mG | |
| RNACS7809 | B2m1_ | mA*mC*mU*rCrArCrGrCrUrGrGrArUrArGrCrCrUrCrCrGrUrUrUrUrA | 17681 |
| | P11R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrUrUrCrUrGrGrCr | |
| | v2 | CrUrGrGrArUrArArCrCrGrGrCrUrArUrCrC*mA*mG*mC | |
| RNACS7810 | B2m1_ | mA*mC*mU*rCrArCrGrCrUrGrGrArUrArGrCrCrUrCrCrGrUrUrUrUrA | 17682 |
| | P10R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrUrUrCrUrGrGrCr | |
| | v2 | CrUrGrGrArUrArArCrCrGrGrCrUrArUrC*mC*mA*mG | |
| RNACS7811 | B2m1_ | mA*mC*mU*rCrArCrGrCrUrGrGrArUrArGrCrCrUrCrCrGrUrUrUrUrA | 17683 |
| | P9R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrUrUrCrUrGrGrCr | |
| | v2 | CrUrGrGrArUrArArCrCrGrGrCrUrArU*mC*mC*mA | |
| RNACS7812 | B2m1_ | mA*mC*mU*rCrArCrGrCrUrGrGrArUrArGrCrCrUrCrCrGrUrUrUrUrA | 17684 |
| | P8R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrUrUrCrUrGrGrCr | |
| | v2 | CrUrGrGrArUrArArCrCrGrGrCrUrA*mU*mC*mC | |
| RNACS7813 | B2m1_ | mA*mC*mU*rCrArCrGrCrUrGrGrArUrArGrCrCrUrCrCrGrUrUrUrUrA | 17685 |
| | P13R24_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrCrUrUrUrCrUrGr | |
| | v2 | GrCrCrUrGrGrArUrArArCrCrGrGrCrUrArUrCrCrArG*mC*mG*mU | |
| RNACS7814 | B2m1_ | mA*mC*mU*rCrArCrGrCrUrGrGrArUrArGrCrCrUrCrCrGrUrUrUrUrA | 17686 |
| | P12R24_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrCrUrUrUrCrUrGr | |
| | v2 | GrCrCrUrGrGrArUrArArCrCrGrGrCrUrArUrCrCrA*mG*mC*mG | |
| RNACS7815 | B2m1_ | mA*mC*mU*rCrArCrGrCrUrGrGrArUrArGrCrCrUrCrCrGrUrUrUrUrA | 17687 |
| | P11R24_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrCrUrUrUrCrUrGr | |
| | v2 | GrCrCrUrGrGrArUrArArCrCrGrGrCrUrArUrCrC*mA*mG*mC | |
| RNACS7816 | B2m1_ | mA*mC*mU*rCrArCrGrCrUrGrGrArUrArGrCrCrUrCrCrGrUrUrUrUrA | 17688 |
| | P10R24_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrCrUrUrUrCrUrGr | |
| | v2 | GrCrCrUrGrGrArUrArArCrCrGrGrCrUrArUrC*mC*mA*mG | |
| RNACS7817 | B2m1_ | mA*mC*mU*rCrArCrGrCrUrGrGrArUrArGrCrCrUrCrCrGrUrUrUrUrA | 17689 |
| | P9R24_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrCrUrUrUrCrUrGr | |
| | v2 | GrCrCrUrGrGrArUrArArCrCrGrGrCrUrArU*mC*mC*mA | |
| RNACS7818 | B2m1_ | mA*mC*mU*rCrArCrGrCrUrGrGrArUrArGrCrCrUrCrCrGrUrUrUrUrA | 17690 |
| | P8R24_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrUrCrUrUrUrCrUrGr | |
| | v2 | GrCrCrUrGrGrArUrArArCrCrGrGrCrUrA*mU*mC*mC | |
| RNACS7819 | B2m2_ | mG*mA*mG*rUrArGrCrGrCrGrArGrCrArCrArGrCrUrArGrUrUrUrUrA | 17691 |
| | P13R11_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrUrArGrUrGrArUrCr | |
| | v1 | UrGrUrGrCrUrCrGrC*mG*mC*mU | |
| RNACS7820 | B2m2_ | mG*mA*mG*rUrArGrCrGrCrGrArGrCrArCrArGrCrUrArGrUrUrUrUrA | 17692 |
| | P12R11_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrUrArGrUrGrArUrCr | |
| | v1 | UrGrUrGrCrUrCrG*mC*mG*mC | |
| RNACS7821 | B2m2_ | mG*mA*mG*rUrArGrCrGrCrGrArGrCrArCrArGrCrUrArGrUrUrUrUrA | 17693 |
| | P11R11_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrUrArGrUrGrArUrCr | |
| | v1 | UrGrUrGrCrUrC*mG*mC*mG | |
| RNACS7822 | B2m2_ | mG*mA*mG*rUrArGrCrGrCrGrArGrCrArCrArGrCrUrArGrUrUrUrUrA | 17694 |
| | P10R11_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrUrArGrUrGrArUrCr | |
| | v1 | UrGrUrGrCrU*mC*mG*mC | |
| RNACS7823 | B2m2_ | mG*mA*mG*rUrArGrCrGrCrGrArGrCrArCrArGrCrUrArGrUrUrUrUrA | 17695 |
| | P9R11_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrUrArGrUrGrArUrCr | |
| | v1 | UrGrUrGrC*mU*mC*mG | |
| RNACS7824 | B2m2_ | mG*mA*mG*rUrArGrCrGrCrGrArGrCrArCrArGrCrUrArGrUrUrUrUrA | 17696 |
| | P8R11_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrUrArGrUrGrArUrCr | |
| | v1 | UrGrUrG*mC*mU*mC | |
| RNACS7825 | B2m2_ | mG*mA*mG*rUrArGrCrGrCrGrArGrCrArCrArGrCrUrArGrUrUrUrUrA | 17697 |
| | P13R13_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrUrArGrUrGr | |
| | v1 | ArUrCrUrGrUrGrCrUrCrGrC*mG*mC*mU | |
| RNACS7826 | B2m2_ | mG*mA*mG*rUrArGrCrGrCrGrArGrCrArCrArGrCrUrArGrUrUrUrUrA | 17698 |
| | P12R13_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrUrArGrUrGrAr | |
| | v1 | UrCrUrGrUrGrCrUrCrG*mC*mG*mC | |
| RNACS7827 | B2m2_ | mG*mA*mG*rUrArGrCrGrCrGrArGrCrArCrArGrCrUrArGrUrUrUrUrA | 17699 |
| | P11R13_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrUrArGrUrGr | |
| | v1 | ArUrCrUrGrUrGrCrUrC*mG*mC*mG | |
| RNACS7828 | B2m2_ | mG*mA*mG*rUrArGrCrGrCrGrArGrCrArCrArGrCrUrArGrUrUrUrUrA | 17700 |
| | P10R13_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrUrArGrUrGrAr | |
| | v1 | UrCrUrGrUrGrCrU*mC*mG*mC | |
| RNACS7829 | B2m2_ | mG*mA*mG*rUrArGrCrGrCrGrArGrCrArCrArGrCrUrArGrUrUrUrUrA | 17701 |
| | P9R13_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrUrArGrUrGrAr | |
| | v1 | UrCrUrGrUrGrC*mU*mC*mG | |
| RNACS7830 | B2m2_ | mG*mA*mG*rUrArGrCrGrCrGrArGrCrArCrArGrCrUrArGrUrUrUrUrA | 17702 |
| | P8R13_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrUrArGrUr | |
| | v1 | GrArUrCrUrGrUrG*mC*mU*mC | |
| RNACS7831 | B2m2_ | mG*mA*mG*rUrArGrCrGrCrGrArGrCrArCrArGrCrUrArGrUrUrUrUrA | 17703 |
| | P13R15_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrCrGrUrGrGrCrCrUrUrArGrUr | |
| | v1 | GrArUrCrUrGrUrGrCrUrCrGrC*mG*mC*mU | |
| RNACS7832 | B2m2_ | mG*mA*mG*rUrArGrCrGrCrGrArGrCrArCrArGrCrUrArGrUrUrUrUrA | 17704 |
| | P12R15_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrCrGrUrGrGrCrCrUrUrArGrUr | |
| | v1 | GrArUrCrUrGrUrGrCrUrCrG*mC*mG*mC | |
| RNACS7833 | B2m2_ | mG*mA*mG*rUrArGrCrGrCrGrArGrCrArCrArGrCrUrArGrUrUrUrUrA | 17705 |
| | P11R15_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrCrGrUrGrGrCrCrUrUrArGrUr | |
| | v1 | GrArUrCrUrGrUrGrCrUrC*mG*mC*mG | |
| RNACS7834 | B2m2_ | mG*mA*mG*rUrArGrCrGrCrGrArGrCrArCrArGrCrUrArGrUrUrUrUrA | 17706 |
| | P10R15_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrCrGrUrGrGrCrCrUrUrArGr | |
| | v1 | UrGrArUrCrUrGrUrGrCrU*mC*mG*mC | |
| RNACS7835 | B2m2_ | mG*mA*mG*rUrArGrCrGrCrGrArGrCrArCrArGrCrUrArGrUrUrUrUrA | 17707 |
| | P9R15_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrCrGrUrGrGrCrCrUrUrArGrUr | |
| | v1 | GrArUrCrUrGrUrGrC*mU*mC*mG | |
| RNACS7836 | B2m2_ | mG*mA*mG*rUrArGrCrGrCrGrArGrCrArCrArGrCrUrArGrUrUrUrUrA | 17708 |
| | P8R15_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrCrGrUrGrGrCrCrUrUrArGrUr | |
| | v1 | GrArUrCrUrGrUrG*mC*mU*mC | |
| RNACS7837 | B2m2_ | mG*mA*mG*rUrArGrCrGrCrGrArGrCrArCrArGrCrUrArGrUrUrUrUrA | 17709 |
| | P13R17_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrCrGrUrGrGrCrCrUrUrAr | |
| | v1 | GrUrGrArUrCrUrGrUrGrCrUrCrGrC*mG*mC*mU | |
| RNACS7838 | B2m2_ | mG*mA*mG*rUrArGrCrGrCrGrArGrCrArCrArGrCrUrArGrUrUrUrUrA | 17710 |
| | P12R17_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrCrGrUrGrGrCrCrUrUrAr | |
| | v1 | GrUrGrArUrCrUrGrUrGrCrUrCrG*mC*mG*mC | |
| RNACS7839 | B2m2_ | mG*mA*mG*rUrArGrCrGrCrGrArGrCrArCrArGrCrUrArGrUrUrUrUrA | 17711 |
| | P11R17_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrCrGrUrGrGrCrCrUrUr | |
| | v1 | ArGrUrGrArUrCrUrGrUrGrCrUrC*mG*mC*mG | |
| RNACS7840 | B2m2_ | mG*mA*mG*rUrArGrCrGrCrGrArGrCrArCrArGrCrUrArGrUrUrUrUrA | 17712 |
| | P10R17_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrCrGrUrGrGrCrCrUrUrAr | |
| | v1 | GrUrGrArUrCrUrGrUrGrCrU*mC*mG*mC | |
| RNACS7841 | B2m2_ | mG*mA*mG*rUrArGrCrGrCrGrArGrCrArCrArGrCrUrArGrUrUrUrUrA | 17713 |
| | P9R17_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrCrGrUrGrGrCrCrUrUrAr | |
| | v1 | GrUrGrArUrCrUrGrUrGrC*mU*mC*mG | |
| RNACS7842 | B2m2_ | mG*mA*mG*rUrArGrCrGrCrGrArGrCrArCrArGrCrUrArGrUrUrUrUrA | 17714 |
| | P8R17_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrCrGrUrGrGrCrCrUrUrAr | |
| | v1 | GrUrGrArUrCrUrGrUrG*mC*mU*mC | |
| RNACS7843 | B2m2_ | mG*mA*mG*rUrArGrCrGrCrGrArGrCrArCrArGrCrUrArGrUrUrUrUrA | 17715 |
| | P13R19_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrUrCrCrGrUrGrGrCrCr | |
| | v1 | UrUrArGrUrGrArUrCrUrGrUrGrCrUrCrGrC*mG*mC*mU | |
| RNACS7844 | B2m2_ | mG*mA*mG*rUrArGrCrGrCrGrArGrCrArCrArGrCrUrArGrUrUrUrUrA | 17716 |
| | P12R19_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrUrCrCrGrUrGrGrCrCr | |
| | v1 | UrUrArGrUrGrArUrCrUrGrUrGrCrUrCrG*mC*mG*mC | |
| RNACS7845 | B2m2_ | mG*mA*mG*rUrArGrCrGrCrGrArGrCrArCrArGrCrUrArGrUrUrUrUrA | 17717 |
| | P11R19_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrUrCrCrGrUrGrGrCrCr | |
| | v1 | UrUrArGrUrGrArUrCrUrGrUrGrCrUrC*mG*mC*mG | |
| RNACS7846 | B2m2_ | mG*mA*mG*rUrArGrCrGrCrGrArGrCrArCrArGrCrUrArGrUrUrUrUrA | 17718 |
| | P10R19_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrUrCrCrGrUrGrGrCrCrUr | |
| | v1 | UrArGrUrGrArUrCrUrGrUrGrCrU*mC*mG*mC | |
| RNACS7847 | B2m2_ | mG*mA*mG*rUrArGrCrGrCrGrArGrCrArCrArGrCrUrArGrUrUrUrUrA | 17719 |
| | P9R19_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrUrCrCrGrUrGrGrCrCr | |
| | v1 | UrUrArGrUrGrArUrCrUrGrUrGrC*mU*mC*mG | |
| RNACS7848 | B2m2_ | mG*mA*mG*rUrArGrCrGrCrGrArGrCrArCrArGrCrUrArGrUrUrUrUrA | 17720 |
| | P8R19_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrUrCrCrGrUrGrGrCrCrUr | |
| | v1 | UrArGrUrGrArUrCrUrGrUrG*mC*mU*mC | |
| RNACS7849 | B2m2_ | mG*mA*mG*rUrArGrCrGrCrGrArGrCrArCrArGrCrUrArGrUrUrUrUrA | 17721 |
| | P13R21_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrGrCrUrCrCrGrUrGrGrCr | |
| | v1 | CrUrUrArGrUrGrArUrCrUrGrUrGrCrUrCrGrC*mG*mC*mU | |
| RNACS7850 | B2m2_ | mG*mA*mG*rUrArGrCrGrCrGrArGrCrArCrArGrCrUrArGrUrUrUrUrA | 17722 |
| | P12R21_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrGrCrUrCrCrGrUrGrGrCr | |
| | v1 | CrUrUrArGrUrGrArUrCrUrGrUrGrCrUrCrG*mC*mG*mC | |
| RNACS7851 | B2m2_ | mG*mA*mG*rUrArGrCrGrCrGrArGrCrArCrArGrCrUrArGrUrUrUrUrA | 17723 |
| | P11R21_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrGrCrUrCrCrGrUrGrGrCr | |
| | v1 | CrUrUrArGrUrGrArUrCrUrGrUrGrCrUrC*mG*mC*mG | |
| RNACS7852 | B2m2_ | mG*mA*mG*rUrArGrCrGrCrGrArGrCrArCrArGrCrUrArGrUrUrUrUrA | 17724 |
| | P10R21_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrGrCrUrCrCrGrUrGrGrCr | |
| | v1 | CrUrUrArGrUrGrArUrCrUrGrUrGrCrU*mC*mG*mC | |
| RNACS7853 | B2m2_ | mG*mA*mG*rUrArGrCrGrCrGrArGrCrArCrArGrCrUrArGrUrUrUrUrA | 17725 |
| | P9R21_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrGrCrUrCrCrGrUrGrGrCr | |
| | v1 | CrUrUrArGrUrGrArUrCrUrGrUrGrC*mU*mC*mG | |
| RNACS7854 | B2m2_ | mG*mA*mG*rUrArGrCrGrCrGrArGrCrArCrArGrCrUrArGrUrUrUrUrA | 17726 |
| | P8R21_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrGrCrUrCrCrGrUrGrGrCr | |
| | v1 | CrUrUrArGrUrGrArUrCrUrGrUrG*mC*mU*mC | |
| RNACS7855 | B2m2_ | mG*mA*mG*rUrArGrCrGrCrGrArGrCrArCrArGrCrUrArGrUrUrUrUrA | 17727 |
| | P13R23_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrGrCrUrCrCrGrUrGr | |
| | v1 | GrCrCrUrUrArGrUrGrArUrCrUrGrUrGrCrUrCrGrC*mG*mC*mU | |
| RNACS7856 | B2m2_ | mG*mA*mG*rUrArGrCrGrCrGrArGrCrArCrArGrCrUrArGrUrUrUrUrA | 17728 |
| | P12R23_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrGrCrUrCrCrGrUrGr | |
| | v1 | GrCrCrUrUrArGrUrGrArUrCrUrGrUrGrCrUrCrG*mC*mG*mC | |
| RNACS7857 | B2m2_ | mG*mA*mG*rUrArGrCrGrCrGrArGrCrArCrArGrCrUrArGrUrUrUrUrA | 17729 |
| | P11R23_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrGrCrUrCrCrGrUrGr | |
| | v1 | GrCrCrUrUrArGrUrGrArUrCrUrGrUrGrCrUrC*mG*mC*mG | |
| RNACS7858 | B2m2_ | mG*mA*mG*rUrArGrCrGrCrGrArGrCrArCrArGrCrUrArGrUrUrUrUrA | 17730 |
| | P10R23_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrGrCrUrCrCrGrUrGr | |
| | v1 | GrCrCrUrUrArGrUrGrArUrCrUrGrUrGrCrU*mC*mG*mC | |
| RNACS7859 | B2m2_ | mG*mA*mG*rUrArGrCrGrCrGrArGrCrArCrArGrCrUrArGrUrUrUrUrA | 17731 |
| | P9R23_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrGrCrUrCrCrGrUrGr | |
| | v1 | GrCrCrUrUrArGrUrGrArUrCrUrGrUrGrC*mU*mC*mG | |
| RNACS7860 | B2m2_ | mG*mA*mG*rUrArGrCrGrCrGrArGrCrArCrArGrCrUrArGrUrUrUrUrA | 17732 |
| | P8R23_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrGrCrUrCrCrGrUrGr | |
| | v1 | GrCrCrUrUrArGrUrGrArUrCrUrGrUrG*mC*mU*mC | |
| RNACS7861 | B2m2_ | mG*mA*mG*rUrArGrCrGrCrGrArGrCrArCrArGrCrUrArGrUrUrUrUrA | 17733 |
| | P13R11_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrUrArGrUrArAr | |
| | v2 | CrCrUrGrUrGrCrUrCrGrC*mG*mC*mU | |
| RNACS7862 | B2m2_ | mG*mA*mG*rUrArGrCrGrCrGrArGrCrArCrArGrCrUrArGrUrUrUrUrA | 17734 |
| | P12R11_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrUrArGrUrArArCrCr | |
| | v2 | UrGrUrGrCrUrCrG*mC*mG*mC | |
| RNACS7863 | B2m2_ | mG*mA*mG*rUrArGrCrGrCrGrArGrCrArCrArGrCrUrArGrUrUrUrUrA | 17735 |
| | P11R11_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrUrArGrUrArArCr | |
| | v2 | CrUrGrUrGrCrUrC*mG*mC*mG | |
| RNACS7864 | B2m2_ | mG*mA*mG*rUrArGrCrGrCrGrArGrCrArCrArGrCrUrArGrUrUrUrUrA | 17736 |
| | P10R11_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrUrArGrUrArArCrCr | |
| | v2 | UrGrUrGrCrU*mC*mG*mC | |
| RNACS7865 | B2m2_ | mG*mA*mG*rUrArGrCrGrCrGrArGrCrArCrArGrCrUrArGrUrUrUrUrA | 17737 |
| | P9R11_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrUrArGrUrArArCrCr | |
| | v2 | UrGrUrGrC*mU*mC*mG | |
| RNACS7866 | B2m2_ | mG*mA*mG*rUrArGrCrGrCrGrArGrCrArCrArGrCrUrArGrUrUrUrUrA | 17738 |
| | P8R11_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrUrArGrUrArArCrCr | |
| | v2 | UrGrUrG*mC*mU*mC | |
| RNACS7867 | B2m2_ | mG*mA*mG*rUrArGrCrGrCrGrArGrCrArCrArGrCrUrArGrUrUrUrUrA | 17739 |
| | P13R13_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrUrArGrUrArAr | |
| | v2 | CrCrUrGrUrGrCrUrCrGrC*mG*mC*mU | |
| RNACS7868 | B2m2_ | mG*mA*mG*rUrArGrCrGrCrGrArGrCrArCrArGrCrUrArGrUrUrUrUrA | 17740 |
| | P12R13_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrUrArGrUrArAr | |
| | v2 | CrCrUrGrUrGrCrUrCrG*mC*mG*mC | |
| RNACS7869 | B2m2_ | mG*mA*mG*rUrArGrCrGrCrGrArGrCrArCrArGrCrUrArGrUrUrUrUrA | 17741 |
| | P11R13_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrUrArGrUrArAr | |
| | v2 | CrCrUrGrUrGrCrUrC*mG*mC*mG | |
| RNACS7870 | B2m2_ | mG*mA*mG*rUrArGrCrGrCrGrArGrCrArCrArGrCrUrArGrUrUrUrUrA | 17742 |
| | P10R13_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrUrArGrUrAr | |
| | v2 | ArCrCrUrGrUrGrCrU*mC*mG*mC | |
| RNACS7871 | B2m2_ | mG*mA*mG*rUrArGrCrGrCrGrArGrCrArCrArGrCrUrArGrUrUrUrUrA | 17743 |
| | P9R13_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrUrArGrUrArAr | |
| | v2 | CrCrUrGrUrGrC*mU*mC*mG | |
| RNACS7872 | B2m2_ | mG*mA*mG*rUrArGrCrGrCrGrArGrCrArCrArGrCrUrArGrUrUrUrUrA | 17744 |
| | P8R13_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrUrArGrUrArAr | |
| | v2 | CrCrUrGrUrG*mC*mU*mC | |
| RNACS7873 | B2m2_ | mG*mA*mG*rUrArGrCrGrCrGrArGrCrArCrArGrCrUrArGrUrUrUrUrA | 17745 |
| | P13R15_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrCrGrUrGrGrCrCrUrUrArGrUr | |
| | v2 | ArArCrCrUrGrUrGrCrUrCrGrC*mG*mC*mU | |
| RNACS7874 | B2m2_ | mG*mA*mG*rUrArGrCrGrCrGrArGrCrArCrArGrCrUrArGrUrUrUrUrA | 17746 |
| | P12R15_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrCrGrUrGrGrCrCrUrUrArGrUr | |
| | v2 | ArArCrCrUrGrUrGrCrUrCrG*mC*mG*mC | |
| RNACS7875 | B2m2_ | mG*mA*mG*rUrArGrCrGrCrGrArGrCrArCrArGrCrUrArGrUrUrUrUrA | 17747 |
| | P11R15_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrCrGrUrGrGrCrCrUrUrArGrUr | |
| | v2 | ArArCrCrUrGrUrGrCrUrC*mG*mC*mG | |
| RNACS7876 | B2m2_ | mG*mA*mG*rUrArGrCrGrCrGrArGrCrArCrArGrCrUrArGrUrUrUrUrA | 17748 |
| | P10R15_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrCrGrUrGrGrCrCrUrUrArGrUr | |
| | v2 | ArArCrCrUrGrUrGrCrU*mC*mG*mC | |
| RNACS7877 | B2m2_ | mG*mA*mG*rUrArGrCrGrCrGrArGrCrArCrArGrCrUrArGrUrUrUrUrA | 17749 |
| | P9R15_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrCrGrUrGrGrCrCrUrUrArGrUr | |
| | v2 | ArArCrCrUrGrUrGrC*mU*mC*mG | |
| RNACS7878 | B2m2_ | mG*mA*mG*rUrArGrCrGrCrGrArGrCrArCrArGrCrUrArGrUrUrUrUrA | 17750 |
| | P8R15_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrCrGrUrGrGrCrCrUrUrArGrUr | |
| | v2 | ArArCrCrUrGrUrG*mC*mU*mC | |
| RNACS7879 | B2m2_ | mG*mA*mG*rUrArGrCrGrCrGrArGrCrArCrArGrCrUrArGrUrUrUrUrA | 17751 |
| | P13R17_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrCrGrUrGrGrCrCrUrUrAr | |
| | v2 | GrUrArArCrCrUrGrUrGrCrUrCrGrC*mG*mC*mU | |
| RNACS7880 | B2m2_ | mG*mA*mG*rUrArGrCrGrCrGrArGrCrArCrArGrCrUrArGrUrUrUrUrA | 17752 |
| | P12R17_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrCrGrUrGrGrCrCrUrUrAr | |
| | v2 | GrUrArArCrCrUrGrUrGrCrUrCrG*mC*mG*mC | |
| RNACS7881 | B2m2_ | mG*mA*mG*rUrArGrCrGrCrGrArGrCrArCrArGrCrUrArGrUrUrUrUrA | 17753 |
| | P11R17_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrCrGrUrGrGrCrCrUrUr | |
| | v2 | ArGrUrArArCrCrUrGrUrGrCrUrC*mG*mC*mG | |
| RNACS7882 | B2m2_ | mG*mA*mG*rUrArGrCrGrCrGrArGrCrArCrArGrCrUrArGrUrUrUrUrA | 17754 |
| | P10R17_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrCrGrUrGrGrCrCrUrUrAr | |
| | v2 | GrUrArArCrCrUrGrUrGrCrU*mC*mG*mC | |
| RNACS7883 | B2m2_ | mG*mA*mG*rUrArGrCrGrCrGrArGrCrArCrArGrCrUrArGrUrUrUrUrA | 17755 |
| | P9R17_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrCrGrUrGrGrCrCrUrUrAr | |
| | v2 | GrUrArArCrCrUrGrUrGrC*mU*mC*mG | |
| RNACS7884 | B2m2_ | mG*mA*mG*rUrArGrCrGrCrGrArGrCrArCrArGrCrUrArGrUrUrUrUrA | 17756 |
| | P8R17_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrCrGrUrGrGrCrCrUrUrAr | |
| | v2 | GrUrArArCrCrUrGrUrG*mC*mU*mC | |
| RNACS7885 | B2m2_ | mG*mA*mG*rUrArGrCrGrCrGrArGrCrArCrArGrCrUrArGrUrUrUrUrA | 17757 |
| | P13R19_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrUrCrCrGrUrGrGrCrCr | |
| | v2 | UrUrArGrUrArArCrCrUrGrUrGrCrUrCrGrC*mG*mC*mU | |
| RNACS7886 | B2m2_ | mG*mA*mG*rUrArGrCrGrCrGrArGrCrArCrArGrCrUrArGrUrUrUrUrA | 17758 |
| | P12R19_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrUrCrCrGrUrGrGrCrCrUr | |
| | v2 | UrArGrUrArArCrCrUrGrUrGrCrUrCrG*mC*mG*mC | |
| RNACS7887 | B2m2_ | mG*mA*mG*rUrArGrCrGrCrGrArGrCrArCrArGrCrUrArGrUrUrUrUrA | 17759 |
| | P11R19_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrUrCrCrGrUrGrGrCrCrUr | |
| | v2 | UrArGrUrArArCrCrUrGrUrGrCrUrC*mG*mC*mG | |
| RNACS7888 | B2m2_ | mG*mA*mG*rUrArGrCrGrCrGrArGrCrArCrArGrCrUrArGrUrUrUrUrA | 17760 |
| | P10R19_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrUrCrCrGrUrGrGrCrCrUr | |
| | v2 | UrArGrUrArArCrCrUrGrUrGrCrU*mC*mG*mC | |
| RNACS7889 | B2m2_ | mG*mA*mG*rUrArGrCrGrCrGrArGrCrArCrArGrCrUrArGrUrUrUrUrA | 17761 |
| | P9R19_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrUrCrCrGrUrGrGrCrCrUr | |
| | v2 | UrArGrUrArArCrCrUrGrUrGrC*mU*mC*mG | |
| RNACS7890 | B2m2_ | mG*mA*mG*rUrArGrCrGrCrGrArGrCrArCrArGrCrUrArGrUrUrUrUrA | 17762 |
| | P8R19_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrUrCrCrGrUrGrGrCrCrUr | |
| | v2 | UrArGrUrArArCrCrUrGrUrG*mC*mU*mC | |
| RNACS7891 | B2m2_ | mG*mA*mG*rUrArGrCrGrCrGrArGrCrArCrArGrCrUrArGrUrUrUrUrA | 17763 |
| | P13R21_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrGrCrUrCrCrGrUrGrGrCr | |
| | v2 | CrUrUrArGrUrArArCrCrUrGrUrGrCrUrCrGrC*mG*mC*mU | |
| RNACS7892 | B2m2_ | mG*mA*mG*rUrArGrCrGrCrGrArGrCrArCrArGrCrUrArGrUrUrUrUrA | 17764 |
| | P12R21_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrGrCrUrCrCrGrUrGrGrCr | |
| | v2 | CrUrUrArGrUrArArCrCrUrGrUrGrCrUrCrG*mC*mG*mC | |
| RNACS7893 | B2m2_ | mG*mA*mG*rUrArGrCrGrCrGrArGrCrArCrArGrCrUrArGrUrUrUrUrA | 17765 |
| | P11R21_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrGrCrUrCrCrGrUrGrGrCr | |
| | v2 | CrUrUrArGrUrArArCrCrUrGrUrGrCrUrC*mG*mC*mG | |
| RNACS7894 | B2m2_ | mG*mA*mG*rUrArGrCrGrCrGrArGrCrArCrArGrCrUrArGrUrUrUrUrA | 17766 |
| | P10R21_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrGrCrUrCrCrGrUrGrGrCr | |
| | v2 | CrUrUrArGrUrArArCrCrUrGrUrGrCrU*mC*mG*mC | |
| RNACS7895 | B2m2_ | mG*mA*mG*rUrArGrCrGrCrGrArGrCrArCrArGrCrUrArGrUrUrUrUrA | 17767 |
| | P9R21_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrGrCrUrCrCrGrUrGrGrCr | |
| | v2 | CrUrUrArGrUrArArCrCrUrGrUrGrC*mU*mC*mG | |
| RNACS7896 | B2m2_ | mG*mA*mG*rUrArGrCrGrCrGrArGrCrArCrArGrCrUrArGrUrUrUrUrA | 17768 |
| | P8R21_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrGrCrUrCrCrGrUrGrGrCr | |
| | v2 | CrUrUrArGrUrArArCrCrUrGrUrG*mC*mU*mC | |
| RNACS7897 | B2m2_ | mG*mA*mG*rUrArGrCrGrCrGrArGrCrArCrArGrCrUrArGrUrUrUrUrA | 17769 |
| | P13R23_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrGrCrUrCrCrGrUrGr | |
| | v2 | GrCrCrUrUrArGrUrArArCrCrUrGrUrGrCrUrCrGrC*mG*mC*mU | |
| RNACS7898 | B2m2_ | mG*mA*mG*rUrArGrCrGrCrGrArGrCrArCrArGrCrUrArGrUrUrUrUrA | 17770 |
| | P12R23_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrGrCrUrCrCrGrUrGr | |
| | v2 | GrCrCrUrUrArGrUrArArCrCrUrGrUrGrCrUrCrG*mC*mG*mC | |
| RNACS7899 | B2m2_ | mG*mA*mG*rUrArGrCrGrCrGrArGrCrArCrArGrCrUrArGrUrUrUrUrA | 17771 |
| | P11R23_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrGrCrUrCrCrGrUrGr | |
| | v2 | GrCrCrUrUrArGrUrArArCrCrUrGrUrGrCrUrC*mG*mC*mG | |
| RNACS7900 | B2m2_ | mG*mA*mG*rUrArGrCrGrCrGrArGrCrArCrArGrCrUrArGrUrUrUrUrA | 17772 |
| | P10R23_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrGrCrUrCrCrGrUr | |
| | v2 | GrGrCrCrUrUrArGrUrArArCrCrUrGrUrGrCrU*mC*mG*mC | |
| RNACS7901 | B2m2_ | mG*mA*mG*rUrArGrCrGrCrGrArGrCrArCrArGrCrUrArGrUrUrUrUrA | 17773 |
| | P9R23_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrGrCrUrCrCrGrUrGr | |
| | v2 | GrCrCrUrUrArGrUrArArCrCrUrGrUrGrC*mU*mC*mG | |
| RNACS7902 | B2m2_ | mG*mA*mG*rUrArGrCrGrCrGrArGrCrArCrArGrCrUrArGrUrUrUrUrA | 17774 |
| | P8R23_ | rGrAmGmCmUmAmGmAmAmAmUmAmGmCrArArGrUrUrArArArArUrArArGr | |
| | ins4_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrGrCrUrCrCrGrUrGr | |
| | v2 | GrCrCrUrUrArGrUrArArCrCrUrGrUrG*mC*mU*mC | |
| RNACS7903 | B2m2_ | mG*mA*mG*rUrArGrCrGrCrGrArGrCrArCrArGrCrUrArGrUrUrUrUrA | 17775 |
| | P13R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrUrArGrUrGrArUrGr | |
| | v1 | CrUrGrUrGrCrUrCrGrC*mG*mC*mU | |
| RNACS7904 | B2m2_ | mG*mA*mG*rUrArGrCrGrCrGrArGrCrArCrArGrCrUrArGrUrUrUrUrA | 17776 |
| | P12R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrUrArGrUrGrArUrGr | |
| | v1 | CrUrGrUrGrCrUrCrG*mC*mG*mC | |
| RNACS7905 | B2m2_ | mG*mA*mG*rUrArGrCrGrCrGrArGrCrArCrArGrCrUrArGrUrUrUrUrA | 17777 |
| | P11R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrUrArGrUrGrArUrGr | |
| | v1 | CrUrGrUrGrCrUrC*mG*mC*mG | |
| RNACS7906 | B2m2_ | mG*mA*mG*rUrArGrCrGrCrGrArGrCrArCrArGrCrUrArGrUrUrUrUrA | 17778 |
| | P10R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrUrArGrUrGrArUrGr | |
| | v1 | CrUrGrUrGrCrU*mC*mG*mC | |
| RNACS7907 | B2m2_ | mG*mA*mG*rUrArGrCrGrCrGrArGrCrArCrArGrCrUrArGrUrUrUrUrA | 17779 |
| | P9R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrUrArGrUrGrArUrGr | |
| | v1 | CrUrGrUrGrC*mU*mC*mG | |
| RNACS7908 | B2m2_ | mG*mA*mG*rUrArGrCrGrCrGrArGrCrArCrArGrCrUrArGrUrUrUrUrA | 17780 |
| | P8R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrUrArGrUrGrArUrGr | |
| | v1 | CrUrGrUrG*mC*mU*mC | |
| RNACS7909 | B2m2_ | mG*mA*mG*rUrArGrCrGrCrGrArGrCrArCrArGrCrUrArGrUrUrUrUrA | 17781 |
| | P13R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrUrArGrUrGr | |
| | v1 | ArUrGrCrUrGrUrGrCrUrCrGrC*mG*mC*mU | |
| RNACS7910 | B2m2_ | mG*mA*mG*rUrArGrCrGrCrGrArGrCrArCrArGrCrUrArGrUrUrUrUrA | 17782 |
| | P12R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrUrArGrUrGrAr | |
| | v1 | UrGrCrUrGrUrGrCrUrCrG*mC*mG*mC | |
| RNACS7911 | B2m2_ | mG*mA*mG*rUrArGrCrGrCrGrArGrCrArCrArGrCrUrArGrUrUrUrUrA | 17783 |
| | P11R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrUrArGrUrGrAr | |
| | v1 | UrGrCrUrGrUrGrCrUrC*mG*mC*mG | |
| RNACS7912 | B2m2_ | mG*mA*mG*rUrArGrCrGrCrGrArGrCrArCrArGrCrUrArGrUrUrUrUrA | 17784 |
| | P10R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrUrArGrUrGrAr | |
| | v1 | UrGrCrUrGrUrGrCrU*mC*mG*mC | |
| RNACS7913 | B2m2_ | mG*mA*mG*rUrArGrCrGrCrGrArGrCrArCrArGrCrUrArGrUrUrUrUrA | 17785 |
| | P9R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrUrArGrUrGrAr | |
| | v1 | UrGrCrUrGrUrGrC*mU*mC*mG | |
| RNACS7914 | B2m2_ | mG*mA*mG*rUrArGrCrGrCrGrArGrCrArCrArGrCrUrArGrUrUrUrUrA | 17786 |
| | P8R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrUrArGrUrGrAr | |
| | v1 | UrGrCrUrGrUrG*mC*mU*mC | |
| RNACS7915 | B2m2_ | mG*mA*mG*rUrArGrCrGrCrGrArGrCrArCrArGrCrUrArGrUrUrUrUrA | 17787 |
| | P13R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrCrGrUrGrGrCrCrUrUrArGrUr | |
| | v1 | GrArUrGrCrUrGrUrGrCrUrCrGrC*mG*mC*mU | |
| RNACS7916 | B2m2_ | mG*mA*mG*rUrArGrCrGrCrGrArGrCrArCrArGrCrUrArGrUrUrUrUrA | 17788 |
| | P12R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrCrGrUrGrGrCrCrUrUrArGrUr | |
| | v1 | GrArUrGrCrUrGrUrGrCrUrCrG*mC*mG*mC | |
| RNACS7917 | B2m2_ | mG*mA*mG*rUrArGrCrGrCrGrArGrCrArCrArGrCrUrArGrUrUrUrUrA | 17789 |
| | P11R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrCrGrUrGrGrCrCrUrUrArGrUr | |
| | v1 | GrArUrGrCrUrGrUrGrCrUrC*mG*mC*mG | |
| RNACS7918 | B2m2_ | mG*mA*mG*rUrArGrCrGrCrGrArGrCrArCrArGrCrUrArGrUrUrUrUrA | 17790 |
| | P10R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrCrGrUrGrGrCrCrUrUrArGr | |
| | v1 | UrGrArUrGrCrUrGrUrGrCrU*mC*mG*mC | |
| RNACS7919 | B2m2_ | mG*mA*mG*rUrArGrCrGrCrGrArGrCrArCrArGrCrUrArGrUrUrUrUrA | 17791 |
| | P9R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrCrGrUrGrGrCrCrUrUrArGr | |
| | v1 | UrGrArUrGrCrUrGrUrGrC*mU*mC*mG | |
| RNACS7920 | B2m2_ | mG*mA*mG*rUrArGrCrGrCrGrArGrCrArCrArGrCrUrArGrUrUrUrUrA | 17792 |
| | P8R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrCrGrUrGrGrCrCrUrUrArGrUr | |
| | v1 | GrArUrGrCrUrGrUrG*mC*mU*mC | |
| RNACS7921 | B2m2_ | mG*mA*mG*rUrArGrCrGrCrGrArGrCrArCrArGrCrUrArGrUrUrUrUrA | 17793 |
| | P13R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrCrGrUrGrGrCrCrUrUrAr | |
| | v1 | GrUrGrArUrGrCrUrGrUrGrCrUrCrGrC*mG*mC*mU | |
| RNACS7922 | B2m2_ | mG*mA*mG*rUrArGrCrGrCrGrArGrCrArCrArGrCrUrArGrUrUrUrUrA | 17794 |
| | P12R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrCrGrUrGrGrCrCrUrUrAr | |
| | v1 | GrUrGrArUrGrCrUrGrUrGrCrUrCrG*mC*mG*mC | |
| RNACS7923 | B2m2_ | mG*mA*mG*rUrArGrCrGrCrGrArGrCrArCrArGrCrUrArGrUrUrUrUrA | 17795 |
| | P11R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrCrGrUrGrGrCrCrUrUrAr | |
| | v1 | GrUrGrArUrGrCrUrGrUrGrCrUrC*mG*mC*mG | |
| RNACS7924 | B2m2_ | mG*mA*mG*rUrArGrCrGrCrGrArGrCrArCrArGrCrUrArGrUrUrUrUrA | 17796 |
| | P10R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrCrGrUrGrGrCrCrUrUr | |
| | v1 | ArGrUrGrArUrGrCrUrGrUrGrCrUmC*mG*mC | |
| RNACS7925 | B2m2_ | mG*mA*mG*rUrArGrCrGrCrGrArGrCrArCrArGrCrUrArGrUrUrUrUrA | 17797 |
| | P9R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrCrGrUrGrGrCrCrUrUrAr | |
| | v1 | GrUrGrArUrGrCrUrGrUrGrC*mU*mC*mG | |
| RNACS7926 | B2m2_ | mG*mA*mG*rUrArGrCrGrCrGrArGrCrArCrArGrCrUrArGrUrUrUrUrA | 17798 |
| | P8R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrCrGrUrGrGrCrCrUrUrAr | |
| | v1 | GrUrGrArUrGrCrUrGrUrG*mC*mU*mC | |
| RNACS7927 | B2m2_ | mG*mA*mG*rUrArGrCrGrCrGrArGrCrArCrArGrCrUrArGrUrUrUrUrA | 17799 |
| | P13R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrUrCrCrGrUrGrGrCrCrUr | |
| | v1 | UrArGrUrGrArUrGrCrUrGrUrGrCrUrCrGrC*mG*mC*mU | |
| RNACS7928 | B2m2_ | mG*mA*mG*rUrArGrCrGrCrGrArGrCrArCrArGrCrUrArGrUrUrUrUrA | 17800 |
| | P12R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrUrCrCrGrUrGrGrCrCr | |
| | v1 | UrUrArGrUrGrArUrGrCrUrGrUrGrCrUrCrG*mC*mG*mC | |
| RNACS7929 | B2m2_ | mG*mA*mG*rUrArGrCrGrCrGrArGrCrArCrArGrCrUrArGrUrUrUrUrA | 17801 |
| | P11R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrUrCrCrGrUrGrGrCrCrUr | |
| | v1 | UrArGrUrGrArUrGrCrUrGrUrGrCrUrC*mG*mC*mG | |
| RNACS7930 | B2m2_ | mG*mA*mG*rUrArGrCrGrCrGrArGrCrArCrArGrCrUrArGrUrUrUrUrA | 17802 |
| | P10R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrUrCrCrGrUrGrGrCrCr | |
| | v1 | UrUrArGrUrGrArUrGrCrUrGrUrGrCrU*mC*mG*mC | |
| RNACS7931 | B2m2_ | mG*mA*mG*rUrArGrCrGrCrGrArGrCrArCrArGrCrUrArGrUrUrUrUrA | 17803 |
| | P9R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrUrCrCrGrUrGrGrCrCrUr | |
| | v1 | UrArGrUrGrArUrGrCrUrGrUrGrC*mU*mC*mG | |
| RNACS7932 | B2m2_ | mG*mA*mG*rUrArGrCrGrCrGrArGrCrArCrArGrCrUrArGrUrUrUrUrA | 17804 |
| | P8R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrUrCrCrGrUrGrGrCrCr | |
| | v1 | UrUrArGrUrGrArUrGrCrUrGrUrG*mC*mU*mC | |
| RNACS7933 | B2m2_ | mG*mA*mG*rUrArGrCrGrCrGrArGrCrArCrArGrCrUrArGrUrUrUrUrA | 17805 |
| | P13R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrGrCrUrCrCrGrUrGrGr | |
| | v1 | CrCrUrUrArGrUrGrArUrGrCrUrGrUrGrCrUrCrGrC*mG*mC*mU | |
| RNACS7934 | B2m2_ | mG*mA*mG*rUrArGrCrGrCrGrArGrCrArCrArGrCrUrArGrUrUrUrUrA | 17806 |
| | P12R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrGrCrUrCrCrGrUrGrGrCr | |
| | v1 | CrUrUrArGrUrGrArUrGrCrUrGrUrGrCrUrCrG*mC*mG*mC | |
| RNACS7935 | B2m2_ | mG*mA*mG*rUrArGrCrGrCrGrArGrCrArCrArGrCrUrArGrUrUrUrUrA | 17807 |
| | P11R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrGrCrUrCrCrGrUrGrGr | |
| | v1 | CrCrUrUrArGrUrGrArUrGrCrUrGrUrGrCrUrC*mG*mC*mG | |
| RNACS7936 | B2m2_ | mG*mA*mG*rUrArGrCrGrCrGrArGrCrArCrArGrCrUrArGrUrUrUrUrA | 17808 |
| | P10R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrGrCrUrCrCrGrUrGrGrCr | |
| | v1 | CrUrUrArGrUrGrArUrGrCrUrGrUrGrCrU*mC*mG*mC | |
| RNACS7937 | B2m2_ | mG*mA*mG*rUrArGrCrGrCrGrArGrCrArCrArGrCrUrArGrUrUrUrUrA | 17809 |
| | P9R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrGrCrUrCrCrGrUrGrGrCr | |
| | v1 | CrUrUrArGrUrGrArUrGrCrUrGrUrGrC*mU*mC*mG | |
| RNACS7938 | B2m2_ | mG*mA*mG*rUrArGrCrGrCrGrArGrCrArCrArGrCrUrArGrUrUrUrUrA | 17810 |
| | P8R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrGrCrUrCrCrGrUrGrGrCr | |
| | v1 | CrUrUrArGrUrGrArUrGrCrUrGrUrG*mC*mU*mC | |
| RNACS7939 | B2m2_ | mG*mA*mG*rUrArGrCrGrCrGrArGrCrArCrArGrCrUrArGrUrUrUrUrA | 17811 |
| | P13R24_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrGrCrUrCrCrGrUrGr | |
| | v1 | GrCrCrUrUrArGrUrGrArUrGrCrUrGrUrGrCrUrCrGrC*mG*mC*mU | |
| RNACS7940 | B2m2_ | mG*mA*mG*rUrArGrCrGrCrGrArGrCrArCrArGrCrUrArGrUrUrUrUrA | 17812 |
| | P12R24_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrGrCrUrCrCrGrUrGr | |
| | v1 | GrCrCrUrUrArGrUrGrArUrGrCrUrGrUrGrCrUrCrG*mC*mG*mC | |
| RNACS7941 | B2m2_ | mG*mA*mG*rUrArGrCrGrCrGrArGrCrArCrArGrCrUrArGrUrUrUrUrA | 17813 |
| | P11R24_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrGrCrUrCrCrGrUrGr | |
| | v1 | GrCrCrUrUrArGrUrGrArUrGrCrUrGrUrGrCrUrC*mG*mC*mG | |
| RNACS7942 | B2m2_ | mG*mA*mG*rUrArGrCrGrCrGrArGrCrArCrArGrCrUrArGrUrUrUrUrA | 17814 |
| | P10R24_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrGrCrUrCrCrGrUrGr | |
| | v1 | GrCrCrUrUrArGrUrGrArUrGrCrUrGrUrGrCrU*mC*mG*mC | |
| RNACS7943 | B2m2_ | mG*mA*mG*rUrArGrCrGrCrGrArGrCrArCrArGrCrUrArGrUrUrUrUrA | 17815 |
| | P9R24_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrGrCrUrCrCrGrUrGr | |
| | v1 | GrCrCrUrUrArGrUrGrArUrGrCrUrGrUrGrC*mU*mC*mG | |
| RNACS7944 | B2m2_ | mG*mA*mG*rUrArGrCrGrCrGrArGrCrArCrArGrCrUrArGrUrUrUrUrA | 17816 |
| | P8R24_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrGrCrUrCrCrGrUrGr | |
| | v1 | GrCrCrUrUrArGrUrGrArUrGrCrUrGrUrG*mC*mU*mC | |
| RNACS7945 | B2m2_ | mG*mA*mG*rUrArGrCrGrCrGrArGrCrArCrArGrCrUrArGrUrUrUrUrA | 17817 |
| | P13R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrUrArGrUrArArCrCr | |
| | v2 | CrUrGrUrGrCrUrCrGrC*mG*mC*mU | |
| RNACS7946 | B2m2_ | mG*mA*mG*rUrArGrCrGrCrGrArGrCrArCrArGrCrUrArGrUrUrUrUrA | 17818 |
| | P12R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrUrArGrUrArArCrCr | |
| | v2 | CrUrGrUrGrCrUrCrG*mC*mG*mC | |
| RNACS7947 | B2m2_ | mG*mA*mG*rUrArGrCrGrCrGrArGrCrArCrArGrCrUrArGrUrUrUrUrA | 17819 |
| | P11R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrUrArGrUrArArCrCr | |
| | v2 | CrUrGrUrGrCrUrC*mG*mC*mG | |
| RNACS7948 | B2m2_ | mG*mA*mG*rUrArGrCrGrCrGrArGrCrArCrArGrCrUrArGrUrUrUrUrA | 17820 |
| | P10R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrUrArGrUrArArCrCr | |
| | v2 | CrUrGrUrGrCrU*mC*mG*mC | |
| RNACS7949 | B2m2_ | mG*mA*mG*rUrArGrCrGrCrGrArGrCrArCrArGrCrUrArGrUrUrUrUrA | 17821 |
| | P9R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrUrArGrUrArArCrCr | |
| | v2 | CrUrGrUrGrC*mU*mC*mG | |
| RNACS7950 | B2m2_ | mG*mA*mG*rUrArGrCrGrCrGrArGrCrArCrArGrCrUrArGrUrUrUrUrA | 17822 |
| | P8R12_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrCrUrUrArGrUrArArCrCr | |
| | v2 | CrUrGrUrG*mC*mU*mC | |
| RNACS7951 | B2m2_ | mG*mA*mG*rUrArGrCrGrCrGrArGrCrArCrArGrCrUrArGrUrUrUrUrA | 17823 |
| | P13R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrUrArGrUrArAr | |
| | v2 | CrCrCrUrGrUrGrCrUrCrGrC*mG*mC*mU | |
| RNACS7952 | B2m2_ | mG*mA*mG*rUrArGrCrGrCrGrArGrCrArCrArGrCrUrArGrUrUrUrUrA | 17824 |
| | P12R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrUrArGrUrArAr | |
| | v2 | CrCrCrUrGrUrGrCrUrCrG*mC*mG*mC | |
| RNACS7953 | B2m2_ | mG*mA*mG*rUrArGrCrGrCrGrArGrCrArCrArGrCrUrArGrUrUrUrUrA | 17825 |
| | P11R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrUrArGrUrArAr | |
| | v2 | CrCrCrUrGrUrGrCrUrC*mG*mC*mG | |
| RNACS7954 | B2m2_ | mG*mA*mG*rUrArGrCrGrCrGrArGrCrArCrArGrCrUrArGrUrUrUrUrA | 17826 |
| | P10R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrUrArGrUrArAr | |
| | v2 | CrCrCrUrGrUrGrCrU*mC*mG*mC | |
| RNACS7955 | B2m2_ | mG*mA*mG*rUrArGrCrGrCrGrArGrCrArCrArGrCrUrArGrUrUrUrUrA | 17827 |
| | P9R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrUrArGrUrArAr | |
| | v2 | CrCrCrUrGrUrGrC*mU*mC*mG | |
| RNACS7956 | B2m2_ | mG*mA*mG*rUrArGrCrGrCrGrArGrCrArCrArGrCrUrArGrUrUrUrUrA | 17828 |
| | P8R14_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrGrGrCrCrUrUrArGrUrArAr | |
| | v2 | CrCrCrUrGrUrG*mC*mU*mC | |
| RNACS7957 | B2m2_ | mG*mA*mG*rUrArGrCrGrCrGrArGrCrArCrArGrCrUrArGrUrUrUrUrA | 17829 |
| | P13R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrCrGrUrGrGrCrCrUrUrArGrUr | |
| | v2 | ArArCrCrCrUrGrUrGrCrUrCrGrC*mG*mC*mU | |
| RNACS7958 | B2m2_ | mG*mA*mG*rUrArGrCrGrCrGrArGrCrArCrArGrCrUrArGrUrUrUrUrA | 17830 |
| | P12R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrCrGrUrGrGrCrCrUrUrArGrUr | |
| | v2 | ArArCrCrCrUrGrUrGrCrUrCrG*mC*mG*mC | |
| RNACS7959 | B2m2_ | mG*mA*mG*rUrArGrCrGrCrGrArGrCrArCrArGrCrUrArGrUrUrUrUrA | 17831 |
| | P11R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrCrGrUrGrGrCrCrUrUrArGrUr | |
| | v2 | ArArCrCrCrUrGrUrGrCrUrC*mG*mC*mG | |
| RNACS7960 | B2m2_ | mG*mA*mG*rUrArGrCrGrCrGrArGrCrArCrArGrCrUrArGrUrUrUrUrA | 17832 |
| | P10R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrCrGrUrGrGrCrCrUrUrArGrUr | |
| | v2 | ArArCrCrCrUrGrUrGrCrU*mC*mG*mC | |
| RNACS7961 | B2m2_ | mG*mA*mG*rUrArGrCrGrCrGrArGrCrArCrArGrCrUrArGrUrUrUrUrA | 17833 |
| | P9R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrCrGrUrGrGrCrCrUrUrArGrUr | |
| | v2 | ArArCrCrCrUrGrUrGrC*mU*mC*mG | |
| RNACS7962 | B2m2_ | mG*mA*mG*rUrArGrCrGrCrGrArGrCrArCrArGrCrUrArGrUrUrUrUrA | 17834 |
| | P8R16_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrCrGrUrGrGrCrCrUrUrArGrUr | |
| | v2 | ArArCrCrCrUrGrUrG*mC*mU*mC | |
| RNACS7963 | B2m2_ | mG*mA*mG*rUrArGrCrGrCrGrArGrCrArCrArGrCrUrArGrUrUrUrUrA | 17835 |
| | P13R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrCrGrUrGrGrCrCrUrUr | |
| | v2 | ArGrUrArArCrCrCrUrGrUrGrCrUrCrGrC*mG*mC*mU | |
| RNACS7964 | B2m2_ | mG*mA*mG*rUrArGrCrGrCrGrArGrCrArCrArGrCrUrArGrUrUrUrUrA | 17836 |
| | P12R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrCrGrUrGrGrCrCrUrUrAr | |
| | v2 | GrUrArArCrCrCrUrGrUrGrCrUrCrG*mC*mG*mC | |
| RNACS7965 | B2m2_ | mG*mA*mG*rUrArGrCrGrCrGrArGrCrArCrArGrCrUrArGrUrUrUrUrA | 17837 |
| | P11R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrCrGrUrGrGrCrCrUrUrAr | |
| | v2 | GrUrArArCrCrCrUrGrUrGrCrUrC*mG*mC*mG | |
| RNACS7966 | B2m2_ | mG*mA*mG*rUrArGrCrGrCrGrArGrCrArCrArGrCrUrArGrUrUrUrUrA | 17838 |
| | P10R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrCrGrUrGrGrCrCrUrUrAr | |
| | v2 | GrUrArArCrCrCrUrGrUrGrCrU*mC*mG*mC | |
| RNACS7967 | B2m2_ | mG*mA*mG*rUrArGrCrGrCrGrArGrCrArCrArGrCrUrArGrUrUrUrUrA | 17839 |
| | P9R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrCrGrUrGrGrCrCrUrUrAr | |
| | v2 | GrUrArArCrCrCrUrGrUrGrC*mU*mC*mG | |
| RNACS7968 | B2m2_ | mG*mA*mG*rUrArGrCrGrCrGrArGrCrArCrArGrCrUrArGrUrUrUrUrA | 17840 |
| | P8R18_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrCrGrUrGrGrCrCrUrUrAr | |
| | v2 | GrUrArArCrCrCrUrGrUrG*mC*mU*mC | |
| RNACS7969 | B2m2_ | mG*mA*mG*rUrArGrCrGrCrGrArGrCrArCrArGrCrUrArGrUrUrUrUrA | 17841 |
| | P13R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrUrCrCrGrUrGrGrCrCrUr | |
| | v2 | UrArGrUrArArCrCrCrUrGrUrGrCrUrCrGrC*mG*mC*mU | |
| RNACS7970 | B2m2_ | mG*mA*mG*rUrArGrCrGrCrGrArGrCrArCrArGrCrUrArGrUrUrUrUrA | 17842 |
| | P12R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrUrCrCrGrUrGrGrCrCrUr | |
| | v2 | UrArGrUrArArCrCrCrUrGrUrGrCrUrCrG*mC*mG*mC | |
| RNACS7971 | B2m2_ | mG*mA*mG*rUrArGrCrGrCrGrArGrCrArCrArGrCrUrArGrUrUrUrUrA | 17843 |
| | P11R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrUrCrCrGrUrGrGrCrCrUr | |
| | v2 | UrArGrUrArArCrCrCrUrGrUrGrCrUrC*mG*mC*mG | |
| RNACS7972 | B2m2_ | mG*mA*mG*rUrArGrCrGrCrGrArGrCrArCrArGrCrUrArGrUrUrUrUrA | 17844 |
| | P10R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrUrCrCrGrUrGrGrCrCr | |
| | v2 | UrUrArGrUrArArCrCrCrUrGrUrGrCrU*mC*mG*mC | |
| RNACS7973 | B2m2_ | mG*mA*mG*rUrArGrCrGrCrGrArGrCrArCrArGrCrUrArGrUrUrUrUrA | 17845 |
| | P9R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrUrCrCrGrUrGrGrCrCrUr | |
| | v2 | UrArGrUrArArCrCrCrUrGrUrGrC*mU*mC*mG | |
| RNACS7974 | B2m2_ | mG*mA*mG*rUrArGrCrGrCrGrArGrCrArCrArGrCrUrArGrUrUrUrUrA | 17846 |
| | P8R20_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrGrCrUrCrCrGrUrGrGrCrCr | |
| | v2 | UrUrArGrUrArArCrCrCrUrGrUrG*mC*mU*mC | |
| RNACS7975 | B2m2_ | mG*mA*mG*rUrArGrCrGrCrGrArGrCrArCrArGrCrUrArGrUrUrUrUrA | 17847 |
| | P13R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrGrCrUrCrCrGrUrGrGrCr | |
| | v2 | CrUrUrArGrUrArArCrCrCrUrGrUrGrCrUrCrGrC*mG*mC*mU | |
| RNACS7976 | B2m2_ | mG*mA*mG*rUrArGrCrGrCrGrArGrCrArCrArGrCrUrArGrUrUrUrUrA | 17848 |
| | P12R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrGrCrUrCrCrGrUrGrGrCr | |
| | v2 | CrUrUrArGrUrArArCrCrCrUrGrUrGrCrUrCrG*mC*mG*mC | |
| RNACS7977 | B2m2_ | mG*mA*mG*rUrArGrCrGrCrGrArGrCrArCrArGrCrUrArGrUrUrUrUrA | 17849 |
| | P11R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrGrCrUrCrCrGrUrGrGrCr | |
| | v2 | CrUrUrArGrUrArArCrCrCrUrGrUrGrCrUrC*mG*mC*mG | |
| RNACS7978 | B2m2_ | mG*mA*mG*rUrArGrCrGrCrGrArGrCrArCrArGrCrUrArGrUrUrUrUrA | 17850 |
| | P10R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrGrCrUrCrCrGrUrGrGrCr | |
| | v2 | CrUrUrArGrUrArArCrCrCrUrGrUrGrCrU*mC*mG*mC | |
| RNACS7979 | B2m2_ | mG*mA*mG*rUrArGrCrGrCrGrArGrCrArCrArGrCrUrArGrUrUrUrUrA | 17851 |
| | P9R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrGrCrUrCrCrGrUrGrGrCr | |
| | v2 | CrUrUrArGrUrArArCrCrCrUrGrUrGrC*mU*mC*mG | |
| RNACS7980 | B2m2_ | mG*mA*mG*rUrArGrCrGrCrGrArGrCrArCrArGrCrUrArGrUrUrUrUrA | 17852 |
| | P8R22_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrGrCrUrCrCrGrUrGrGrCr | |
| | v2 | CrUrUrArGrUrArArCrCrCrUrGrUrG*mC*mU*mC | |
| RNACS7981 | B2m2_ | mG*mA*mG*rUrArGrCrGrCrGrArGrCrArCrArGrCrUrArGrUrUrUrUrA | 17853 |
| | P13R24_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrGrCrUrCrCrGrUrGr | |
| | v2 | GrCrCrUrUrArGrUrArArCrCrCrUrGrUrGrCrUrCrGrC*mG*mC*mU | |
| RNACS7982 | B2m2_ | mG*mA*mG*rUrArGrCrGrCrGrArGrCrArCrArGrCrUrArGrUrUrUrUrA | 17854 |
| | P12R24_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrGrCrUrCrCrGrUrGr | |
| | v2 | GrCrCrUrUrArGrUrArArCrCrCrUrGrUrGrCrUrCrG*mC*mG*mC | |
| RNACS7983 | B2m2_ | mG*mA*mG*rUrArGrCrGrCrGrArGrCrArCrArGrCrUrArGrUrUrUrUrA | 17855 |
| | P11R24_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrGrCrUrCrCrGrUrGr | |
| | v2 | GrCrCrUrUrArGrUrArArCrCrCrUrGrUrGrCrUrC*mG*mC*mG | |
| RNACS7984 | B2m2_ | mG*mA*mG*rUrArGrCrGrCrGrArGrCrArCrArGrCrUrArGrUrUrUrUrA | 17856 |
| | P10R24_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrGrCrUrCrCrGrUrGr | |
| | v2 | GrCrCrUrUrArGrUrArArCrCrCrUrGrUrGrCrU*mC*mG*mC | |
| RNACS7985 | B2m2_ | mG*mA*mG*rUrArGrCrGrCrGrArGrCrArCrArGrCrUrArGrUrUrUrUrA | 17857 |
| | P9R24_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrGrCrUrCrCrGrUrGr | |
| | v2 | GrCrCrUrUrArGrUrArArCrCrCrUrGrUrGrC*mU*mC*mG | |
| RNACS7986 | B2m2_ | mG*mA*mG*rUrArGrCrGrCrGrArGrCrArCrArGrCrUrArGrUrUrUrUrA | 17858 |
| | P8R24_ | rGrAmGmCmUmAmGmAmAmAmUmAmGmCrArArGrUrUrArArArArUrArArGr | |
| | ins5_ | GrCrUrArGrUrCrCrGrUrUrArUrCrAmAmCmUmUmGmAmAmAmAmAmGmUmG | |
| | nick_ | mGmCmAmCmCmGmAmGmUmCmGmGmUmGmCrUrCrUrCrGrCrUrCrCrGrUrGr | |
| | v2 | GrCrCrUrUrArGrUrArArCrCrCrUrGrUrG*mC*mU*mC | |

TABLE 2B
Table 1B Sequences without Chemical Modifications
Table 1B provides exemplary template RNAs for use in combination within a heterologous gene modifying system described herein
for disrupting, the B2M gene using a B2M1 gRNA spacer (acUcacgcUggaUagccUcc SEQ ID NO. 18195) or a B2M2 gRNA spacer
(gagUagcgcgagcacagcUa SEQ ID NO. 18196). The description of each exemplary template RNA (column 2) identifies the gRNA
spacer, the length of the PBS, the length of the heterologous object sequence, the nature and size of the edit, and the location in the
target nucleic acid sequence. For exampleB2m1_P13R11_ins4_nick_v1 indicates that the template RNA comprised a B2M1 gRNA
spacer, a PBS of length 13 nucleotides, an RT region (heterologous object sequence and insertion/deletion) of length 11 nucleotides,
and was designed to produce an insertion of 4 nucleotides near the nick.

| Identifier | Description | Sequence | SEQ ID NO |
|---|---|---|---|
| RNACS7 | B2m1_ | ACUCACGCUGGAUAGCCUCCGUUUUAGAGCUAGAAAUAGCAAGUUAAAAUAAGGCUAG | 17859 |
| 651 | P13R11_ins4_ | UCCGUUAUCAACUUGAAAAAGUGGCACCGAGUCGGUGCGCCUGGAUGAUGGCUAUCCA | |
| | nick_v1 | GCGU | |
| RNACS7 | B2m1_ | ACUCACGCUGGAUAGCCUCCGUUUUAGAGCUAGAAAUAGCAAGUUAAAAUAAGGCUAG | 17860 |
| 652 | P12R11_ins4_ | UCCGUUAUCAACUUGAAAAAGUGGCACCGAGUCGGUGCGCCUGGAUGAUGGCUAUCCA | |
| | nick_v1 | GCG | |
| RNACS7 | B2m1_ | ACUCACGCUGGAUAGCCUCCGUUUUAGAGCUAGAAAUAGCAAGUUAAAAUAAGGCUAG | 17861 |
| 653 | P11R11_ins4_ | UCCGUUAUCAACUUGAAAAAGUGGCACCGAGUCGGUGCGCCUGGAUGAUGGCUAUCCA | |
| | nick_v1 | GC | |
| RNACS7 | B2m1_ | ACUCACGCUGGAUAGCCUCCGUUUUAGAGCUAGAAAUAGCAAGUUAAAAUAAGGCUAG | 17862 |
| 654 | P10R11_ins4_ | UCCGUUAUCAACUUGAAAAAGUGGCACCGAGUCGGUGCGCCUGGAUGAUGGCUAUCCA | |
| | nick_v1 | G | |
| RNACS7 | B2m1_ | ACUCACGCUGGAUAGCCUCCGUUUUAGAGCUAGAAAUAGCAAGUUAAAAUAAGGCUAG | 17863 |
| 655 | P9R11_ins4_ | UCCGUUAUCAACUUGAAAAAGUGGCACCGAGUCGGUGCGCCUGGAUGAUGGCUAUCCA | |
| | nick_v1 | | |
| RNACS7 | B2m1_ | ACUCACGCUGGAUAGCCUCCGUUUUAGAGCUAGAAAUAGCAAGUUAAAAUAAGGCUAG | 17864 |
| 656 | P8R11_ins4_ | UCCGUUAUCAACUUGAAAAAGUGGCACCGAGUCGGUGCGCCUGGAUGAUGGCUAUCC | |
| | nick_v1 | | |
| RNACS7 | B2m1_ | ACUCACGCUGGAUAGCCUCCGUUUUAGAGCUAGAAAUAGCAAGUUAAAAUAAGGCUAG | 17865 |
| 657 | P13R13_ins4_ | UCCGUUAUCAACUUGAAAAAGUGGCACCGAGUCGGUGCUGGCCUGGAUGAUGGCUAUC | |
| | nick_v1 | CAGCGU | |
| RNACS7 | B2m1_ | ACUCACGCUGGAUAGCCUCCGUUUUAGAGCUAGAAAUAGCAAGUUAAAAUAAGGCUAG | 17866 |
| 658 | P12R13_ins4_ | UCCGUUAUCAACUUGAAAAAGUGGCACCGAGUCGGUGCUGGCCUGGAUGAUGGCUAUC | |
| | nick_v1 | CAGCG | |
| RNACS7 | B2m1_ | ACUCACGCUGGAUAGCCUCCGUUUUAGAGCUAGAAAUAGCAAGUUAAAAUAAGGCUAG | 17867 |
| 659 | P11R13_ins4_ | UCCGUUAUCAACUUGAAAAAGUGGCACCGAGUCGGUGCUGGCCUGGAUGAUGGCUAUC | |
| | nick_v1 | CAGC | |
| RNACS7 | B2m1_ | ACUCACGCUGGAUAGCCUCCGUUUUAGAGCUAGAAAUAGCAAGUUAAAAUAAGGCUAG | 17868 |
| 660 | P10R13_ins4_ | UCCGUUAUCAACUUGAAAAAGUGGCACCGAGUCGGUGCUGGCCUGGAUGAUGGCUAUC | |
| | nick_v1 | CAG | |
| RNACS7 | B2m1_ | ACUCACGCUGGAUAGCCUCCGUUUUAGAGCUAGAAAUAGCAAGUUAAAAUAAGGCUAG | 17869 |
| 661 | P9R13_ins4_ | UCCGUUAUCAACUUGAAAAAGUGGCACCGAGUCGGUGCUGGCCUGGAUGAUGGCUAUC | |
| | nick_v1 | CA | |
| RNACS7 | B2m1_ | ACUCACGCUGGAUAGCCUCCGUUUUAGAGCUAGAAAUAGCAAGUUAAAAUAAGGCUAG | 17870 |
| 662 | P8R13_ins4_ | UCCGUUAUCAACUUGAAAAAGUGGCACCGAGUCGGUGCUGGCCUGGAUGAUGGCUAUC | |
| | nick_v1 | C | |
| RNACS7 | B2m1_ | ACUCACGCUGGAUAGCCUCCGUUUUAGAGCUAGAAAUAGCAAGUUAAAAUAAGGCUAG | 17871 |
| 663 | P13R15_ins4_ | UCCGUUAUCAACUUGAAAAAGUGGCACCGAGUCGGUGCUCUGGCCUGGAUGAUGGCUA | |
| | nick_v1 | UCCAGCGU | |
| RNACS7 | B2m1_ | ACUCACGCUGGAUAGCCUCCGUUUUAGAGCUAGAAAUAGCAAGUUAAAAUAAGGCUAG | 17872 |
| 664 | P12R15_ins4_ | UCCGUUAUCAACUUGAAAAAGUGGCACCGAGUCGGUGCUCUGGCCUGGAUGAUGGCUA | |
| | nick_v1 | UCCAGCG | |
| RNACS7 | B2m1_ | ACUCACGCUGGAUAGCCUCCGUUUUAGAGCUAGAAAUAGCAAGUUAAAAUAAGGCUAG | 17873 |
| 665 | P11R15_ins4_ | UCCGUUAUCAACUUGAAAAAGUGGCACCGAGUCGGUGCUCUGGCCUGGAUGAUGGCUA | |
| | nick_v1 | UCCAGC | |
| RNACS7 | B2m1_ | ACUCACGCUGGAUAGCCUCCGUUUUAGAGCUAGAAAUAGCAAGUUAAAAUAAGGCUAG | 17874 |
| 666 | P10R15_ins4_ | UCCGUUAUCAACUUGAAAAAGUGGCACCGAGUCGGUGCUCUGGCCUGGAUGAUGGCUA | |
| | nick_v1 | UCCAG | |
| RNACS7 | B2m1_ | ACUCACGCUGGAUAGCCUCCGUUUUAGAGCUAGAAAUAGCAAGUUAAAAUAAGGCUAG | 17875 |
| 667 | P9R15_ins4_ | UCCGUUAUCAACUUGAAAAAGUGGCACCGAGUCGGUGCUCUGGCCUGGAUGAUGGCUA | |
| | nick_v1 | UCCA | |
| RNACS7 | B2m1_ | ACUCACGCUGGAUAGCCUCCGUUUUAGAGCUAGAAAUAGCAAGUUAAAAUAAGGCUAG | 17876 |
| 668 | P8R15_ins4_ | UCCGUUAUCAACUUGAAAAAGUGGCACCGAGUCGGUGCUCUGGCCUGGAUGAUGGCUA | |
| | nick_v1 | UCC | |
| RNACS7 | B2m1_ | ACUCACGCUGGAUAGCCUCCGUUUUAGAGCUAGAAAUAGCAAGUUAAAAUAAGGCUAG | 17877 |
| 669 | P13R17_ins4_ | UCCGUUAUCAACUUGAAAAAGUGGCACCGAGUCGGUGCUUUCUGGCCUGGAUGAUGGC | |
| | nick_v1 | UAUCCAGCGU | |
| RNACS7 | B2m1_ | ACUCACGCUGGAUAGCCUCCGUUUUAGAGCUAGAAAUAGCAAGUUAAAAUAAGGCUAG | 17878 |
| 670 | P12R17_ins4_ | UCCGUUAUCAACUUGAAAAAGUGGCACCGAGUCGGUGCUUUCUGGCCUGGAUGAUGGC | |
| | nick_v1 | UAUCCAGCG | |
| RNACS7 | B2m1_ | ACUCACGCUGGAUAGCCUCCGUUUUAGAGCUAGAAAUAGCAAGUUAAAAUAAGGCUAG | 17879 |
| 671 | P11R17_ins4_ | UCCGUUAUCAACUUGAAAAAGUGGCACCGAGUCGGUGCUUUCUGGCCUGGAUGAUGGC | |
| | nick_v1 | UAUCCAGC | |
| RNACS7 | B2m1_ | ACUCACGCUGGAUAGCCUCCGUUUUAGAGCUAGAAAUAGCAAGUUAAAAUAAGGCUAG | 17880 |
| 672 | P10R17_ins4_ | UCCGUUAUCAACUUGAAAAAGUGGCACCGAGUCGGUGCUUUCUGGCCUGGAUGAUGGC | |
| | nick_v1 | UAUCCAG | |
| RNACS7 | B2m1_ | ACUCACGCUGGAUAGCCUCCGUUUUAGAGCUAGAAAUAGCAAGUUAAAAUAAGGCUAG | 17881 |
| 673 | P9R17_ins4_ | UCCGUUAUCAACUUGAAAAAGUGGCACCGAGUCGGUGCUUUCUGGCCUGGAUGAUGGC | |
| | nick_v1 | UAUCCA | |
| RNACS7 | B2m1_ | ACUCACGCUGGAUAGCCUCCGUUUUAGAGCUAGAAAUAGCAAGUUAAAAUAAGGCUAG | 17882 |
| 674 | P8R17_ins4_ | UCCGUUAUCAACUUGAAAAAGUGGCACCGAGUCGGUGCUUUCUGGCCUGGAUGAUGGC | |
| | nick_v1 | UAUCC | |
| RNACS7 | B2m1_ | ACUCACGCUGGAUAGCCUCCGUUUUAGAGCUAGAAAUAGCAAGUUAAAAUAAGGCUAG | 17883 |
| 675 | P13R19_ins4_ | UCCGUUAUCAACUUGAAAAAGUGGCACCGAGUCGGUGCUCUUUCUGGCCUGGAUGAUG | |
| | nick_v1 | GCUAUCCAGCGU | |
| RNACS7 | B2m1_ | ACUCACGCUGGAUAGCCUCCGUUUUAGAGCUAGAAAUAGCAAGUUAAAAUAAGGCUAG | 17884 |
| 676 | P12R19_ins4_ | UCCGUUAUCAACUUGAAAAAGUGGCACCGAGUCGGUGCUCUUUCUGGCCUGGAUGAUG | |
| | nick_v1 | GCUAUCCAGCG | |
| RNACS7 | B2m1_ | ACUCACGCUGGAUAGCCUCCGUUUUAGAGCUAGAAAUAGCAAGUUAAAAUAAGGCUAG | 17885 |
| 677 | P11R19_ins4_ | UCCGUUAUCAACUUGAAAAAGUGGCACCGAGUCGGUGCUCUUUCUGGCCUGGAUGAUG | |
| | nick_v1 | GCUAUCCAGC | |
| RNACS7 | B2m1_ | ACUCACGCUGGAUAGCCUCCGUUUUAGAGCUAGAAAUAGCAAGUUAAAAUAAGGCUAG | 17886 |
| 678 | P10R19_ins4_ | UCCGUUAUCAACUUGAAAAAGUGGCACCGAGUCGGUGCUCUUUCUGGCCUGGAUGAUG | |
| | nick_v1 | GCUAUCCAG | |
| RNACS7 | B2m1_ | ACUCACGCUGGAUAGCCUCCGUUUUAGAGCUAGAAAUAGCAAGUUAAAAUAAGGCUAG | 17887 |
| 679 | P9R19_ins4_ | UCCGUUAUCAACUUGAAAAAGUGGCACCGAGUCGGUGCUCUUUCUGGCCUGGAUGAUG | |
| | nick_v1 | GCUAUCCA | |
| RNACS7 | B2m1_ | ACUCACGCUGGAUAGCCUCCGUUUUAGAGCUAGAAAUAGCAAGUUAAAAUAAGGCUAG | 17888 |
| 680 | P8R19_ins4_ | UCCGUUAUCAACUUGAAAAAGUGGCACCGAGUCGGUGCUCUUUCUGGCCUGGAUGAUG | |
| | nick_v1 | GCUAUCC | |
| RNACS7 | B2m1_ | ACUCACGCUGGAUAGCCUCCGUUUUAGAGCUAGAAAUAGCAAGUUAAAAUAAGGCUAG | 17889 |
| 681 | P13R21_ins4_ | UCCGUUAUCAACUUGAAAAAGUGGCACCGAGUCGGUGCUCUCUUUCUGGCCUGGAUGA | |
| | nick_v1 | UGGCUAUCCAGCGU | |
| RNACS7 | B2m1_ | ACUCACGCUGGAUAGCCUCCGUUUUAGAGCUAGAAAUAGCAAGUUAAAAUAAGGCUAG | 17890 |
| 682 | P12R21_ins4_ | UCCGUUAUCAACUUGAAAAAGUGGCACCGAGUCGGUGCUCUCUUUCUGGCCUGGAUGA | |
| | nick_v1 | UGGCUAUCCAGCG | |
| RNACS7 | B2m1_ | ACUCACGCUGGAUAGCCUCCGUUUUAGAGCUAGAAAUAGCAAGUUAAAAUAAGGCUAG | 17891 |
| 683 | P11R21_ins4_ | UCCGUUAUCAACUUGAAAAAGUGGCACCGAGUCGGUGCUCUCUUUCUGGCCUGGAUGA | |
| | nick_v1 | UGGCUAUCCAGC | |
| RNACS7 | B2m1_ | ACUCACGCUGGAUAGCCUCCGUUUUAGAGCUAGAAAUAGCAAGUUAAAAUAAGGCUAG | 17892 |
| 684 | P10R21_ins4_ | UCCGUUAUCAACUUGAAAAAGUGGCACCGAGUCGGUGCUCUCUUUCUGGCCUGGAUGA | |
| | nick_v1 | UGGCUAUCCAG | |
| RNACS7 | B2m1_ | ACUCACGCUGGAUAGCCUCCGUUUUAGAGCUAGAAAUAGCAAGUUAAAAUAAGGCUAG | 17893 |
| 685 | P9R21_ins4_ | UCCGUUAUCAACUUGAAAAAGUGGCACCGAGUCGGUGCUCUCUUUCUGGCCUGGAUGA | |
| | nick_v1 | UGGCUAUCCA | |
| RNACS7 | B2m1_ | ACUCACGCUGGAUAGCCUCCGUUUUAGAGCUAGAAAUAGCAAGUUAAAAUAAGGCUAG | 17894 |
| 686 | P8R21_ins4_ | UCCGUUAUCAACUUGAAAAAGUGGCACCGAGUCGGUGCUCUCUUUCUGGCCUGGAUGA | |
| | nick_v1 | UGGCUAUCC | |
| RNACS7 | B2m1_ | ACUCACGCUGGAUAGCCUCCGUUUUAGAGCUAGAAAUAGCAAGUUAAAAUAAGGCUAG | 17895 |
| 687 | P13R23_ins4_ | UCCGUUAUCAACUUGAAAAAGUGGCACCGAGUCGGUGCUCUCUCUUUCUGGCCUGGAU | |
| | nick_v1 | GAUGGCUAUCCAGCGU | |
| RNACS7 | B2m1_ | ACUCACGCUGGAUAGCCUCCGUUUUAGAGCUAGAAAUAGCAAGUUAAAAUAAGGCUAG | 17896 |
| 688 | P12R23_ins4_ | UCCGUUAUCAACUUGAAAAAGUGGCACCGAGUCGGUGCUCUCUCUUUCUGGCCUGGAU | |
| | nick_v1 | GAUGGCUAUCCAGCG | |
| RNACS7 | B2m1_ | ACUCACGCUGGAUAGCCUCCGUUUUAGAGCUAGAAAUAGCAAGUUAAAAUAAGGCUAG | 17897 |
| 689 | P11R23_ins4_ | UCCGUUAUCAACUUGAAAAAGUGGCACCGAGUCGGUGCUCUCUCUUUCUGGCCUGGAU | |
| | nick_v1 | GAUGGCUAUCCAGC | |
| RNACS7 | B2m1_ | ACUCACGCUGGAUAGCCUCCGUUUUAGAGCUAGAAAUAGCAAGUUAAAAUAAGGCUAG | 17898 |
| 690 | P10R23_ins4_ | UCCGUUAUCAACUUGAAAAAGUGGCACCGAGUCGGUGCUCUCUCUUUCUGGCCUGGAU | |
| | nick_v1 | GAUGGCUAUCCAG | |
| RNACS7 | B2m1_ | ACUCACGCUGGAUAGCCUCCGUUUUAGAGCUAGAAAUAGCAAGUUAAAAUAAGGCUAG | 17899 |
| 691 | P9R23_ins4_ | UCCGUUAUCAACUUGAAAAAGUGGCACCGAGUCGGUGCUCUCUCUUUCUGGCCUGGAU | |
| | nick_v1 | GAUGGCUAUCCA | |
| RNACS7 | B2m1_ | ACUCACGCUGGAUAGCCUCCGUUUUAGAGCUAGAAAUAGCAAGUUAAAAUAAGGCUAG | 17900 |
| 692 | P8R23_ins4_ | UCCGUUAUCAACUUGAAAAAGUGGCACCGAGUCGGUGCUCUCUCUUUCUGGCCUGGAU | |
| | nick_v1 | GAUGGCUAUCC | |
| RNACS7 | B2m1_ | ACUCACGCUGGAUAGCCUCCGUUUUAGAGCUAGAAAUAGCAAGUUAAAAUAAGGCUAG | 17901 |
| 693 | P13R11_ins4_ | UCCGUUAUCAACUUGAAAAAGUGGCACCGAGUCGGUGCGCCUGGAUAACGGCUAUCCA | |
| | nick_v2 | GCGU | |
| RNACS7 | B2m1_ | ACUCACGCUGGAUAGCCUCCGUUUUAGAGCUAGAAAUAGCAAGUUAAAAUAAGGCUAG | 17902 |
| 694 | P12R11_ins4_ | UCCGUUAUCAACUUGAAAAAGUGGCACCGAGUCGGUGCGCCUGGAUAACGGCUAUCCA | |
| | nick_v2 | GCG | |
| RNACS7 | B2m1_ | ACUCACGCUGGAUAGCCUCCGUUUUAGAGCUAGAAAUAGCAAGUUAAAAUAAGGCUAG | 17903 |
| 695 | P11R11_ins4_ | UCCGUUAUCAACUUGAAAAAGUGGCACCGAGUCGGUGCGCCUGGAUAACGGCUAUCCA | |
| | nick_v2 | GC | |
| RNACS7 | B2m1_ | ACUCACGCUGGAUAGCCUCCGUUUUAGAGCUAGAAAUAGCAAGUUAAAAUAAGGCUAG | 17904 |
| 696 | P10R11_ins4_ | UCCGUUAUCAACUUGAAAAAGUGGCACCGAGUCGGUGCGCCUGGAUAACGGCUAUCCA | |
| | nick_v2 | G | |
| RNACS7 | B2m1_ | ACUCACGCUGGAUAGCCUCCGUUUUAGAGCUAGAAAUAGCAAGUUAAAAUAAGGCUAG | 17905 |
| 697 | P9R11_ins4_ | UCCGUUAUCAACUUGAAAAAGUGGCACCGAGUCGGUGCGCCUGGAUAACGGCUAUCCA | |
| | nick_v2 | | |
| RNACS7 | B2m1_ | ACUCACGCUGGAUAGCCUCCGUUUUAGAGCUAGAAAUAGCAAGUUAAAAUAAGGCUAG | 17906 |
| 698 | P8R11_ins4_ | UCCGUUAUCAACUUGAAAAAGUGGCACCGAGUCGGUGCGCCUGGAUAACGGCUAUCC | |
| | nick_v2 | | |
| RNACS7 | B2m1_ | ACUCACGCUGGAUAGCCUCCGUUUUAGAGCUAGAAAUAGCAAGUUAAAAUAAGGCUAG | 17907 |
| 699 | P13R13_ins4_ | UCCGUUAUCAACUUGAAAAAGUGGCACCGAGUCGGUGCUGGCCUGGAUAACGGCUAUC | |
| | nick_v2 | CAGCGU | |
| RNACS7 | B2m1_ | ACUCACGCUGGAUAGCCUCCGUUUUAGAGCUAGAAAUAGCAAGUUAAAAUAAGGCUAG | 17908 |
| 700 | P12R13_ins4_ | UCCGUUAUCAACUUGAAAAAGUGGCACCGAGUCGGUGCUGGCCUGGAUAACGGCUAUC | |
| | nick_v2 | CAGCG | |
| RNACS7 | B2m1_ | ACUCACGCUGGAUAGCCUCCGUUUUAGAGCUAGAAAUAGCAAGUUAAAAUAAGGCUAG | 17909 |
| 701 | P11R13_ins4_ | UCCGUUAUCAACUUGAAAAAGUGGCACCGAGUCGGUGCUGGCCUGGAUAACGGCUAUC | |
| | nick_v2 | CAGC | |
| RNACS7 | B2m1_ | ACUCACGCUGGAUAGCCUCCGUUUUAGAGCUAGAAAUAGCAAGUUAAAAUAAGGCUAG | 17910 |
| 702 | P10R13_ins4_ | UCCGUUAUCAACUUGAAAAAGUGGCACCGAGUCGGUGCUGGCCUGGAUAACGGCUAUC | |
| | nick_v2 | CAG | |
| RNACS7 | B2m1_ | ACUCACGCUGGAUAGCCUCCGUUUUAGAGCUAGAAAUAGCAAGUUAAAAUAAGGCUAG | 17911 |
| 703 | P9R13_ins4_ | UCCGUUAUCAACUUGAAAAAGUGGCACCGAGUCGGUGCUGGCCUGGAUAACGGCUAUC | |
| | nick_v2 | CA | |
| RNACS7 | B2m1_ | ACUCACGCUGGAUAGCCUCCGUUUUAGAGCUAGAAAUAGCAAGUUAAAAUAAGGCUAG | 17912 |
| 704 | P8R13_ins4_ | UCCGUUAUCAACUUGAAAAAGUGGCACCGAGUCGGUGCUGGCCUGGAUAACGGCUAUC | |
| | nick_v2 | C | |
| RNACS7 | B2m1_ | ACUCACGCUGGAUAGCCUCCGUUUUAGAGCUAGAAAUAGCAAGUUAAAAUAAGGCUAG | 17913 |
| 705 | P13R15_ins4_ | UCCGUUAUCAACUUGAAAAAGUGGCACCGAGUCGGUGCUCUGGCCUGGAUAACGGCUA | |
| | nick_v2 | UCCAGCGU | |
| RNACS7 | B2m1_ | ACUCACGCUGGAUAGCCUCCGUUUUAGAGCUAGAAAUAGCAAGUUAAAAUAAGGCUAG | 17914 |
| 706 | P12R15_ins4_ | UCCGUUAUCAACUUGAAAAAGUGGCACCGAGUCGGUGCUCUGGCCUGGAUAACGGCUA | |
| | nick_v2 | UCCAGCG | |
| RNACS7 | B2m1_ | ACUCACGCUGGAUAGCCUCCGUUUUAGAGCUAGAAAUAGCAAGUUAAAAUAAGGCUAG | 17915 |
| 707 | P11R15_ins4_ | UCCGUUAUCAACUUGAAAAAGUGGCACCGAGUCGGUGCUCUGGCCUGGAUAACGGCUA | |
| | nick_v2 | UCCAGC | |
| RNACS7 | B2m1_ | ACUCACGCUGGAUAGCCUCCGUUUUAGAGCUAGAAAUAGCAAGUUAAAAUAAGGCUAG | 17916 |
| 708 | P10R15_ins4_ | UCCGUUAUCAACUUGAAAAAGUGGCACCGAGUCGGUGCUCUGGCCUGGAUAACGGCUA | |
| | nick_v2 | UCCAG | |
| RNACS7 | B2m1_ | ACUCACGCUGGAUAGCCUCCGUUUUAGAGCUAGAAAUAGCAAGUUAAAAUAAGGCUAG | 17917 |
| 709 | P9R15_ins4_ | UCCGUUAUCAACUUGAAAAAGUGGCACCGAGUCGGUGCUCUGGCCUGGAUAACGGCUA | |
| | nick_v2 | UCCA | |
| RNACS7 | B2m1_ | ACUCACGCUGGAUAGCCUCCGUUUUAGAGCUAGAAAUAGCAAGUUAAAAUAAGGCUAG | 17918 |
| 710 | P8R15_ins4_ | UCCGUUAUCAACUUGAAAAAGUGGCACCGAGUCGGUGCUCUGGCCUGGAUAACGGCUA | |
| | nick_v2 | UCC | |
| RNACS7 | B2m1_ | ACUCACGCUGGAUAGCCUCCGUUUUAGAGCUAGAAAUAGCAAGUUAAAAUAAGGCUAG | 17919 |
| 711 | P13R17_ins4_ | UCCGUUAUCAACUUGAAAAAGUGGCACCGAGUCGGUGCUUUCUGGCCUGGAUAACGGC | |
| | nick_v2 | UAUCCAGCGU | |
| RNACS7 | B2m1_ | ACUCACGCUGGAUAGCCUCCGUUUUAGAGCUAGAAAUAGCAAGUUAAAAUAAGGCUAG | 17920 |
| 712 | P12R17_ins4_ | UCCGUUAUCAACUUGAAAAAGUGGCACCGAGUCGGUGCUUUCUGGCCUGGAUAACGGC | |
| | nick_v2 | UAUCCAGCG | |
| RNACS7 | B2m1_ | ACUCACGCUGGAUAGCCUCCGUUUUAGAGCUAGAAAUAGCAAGUUAAAAUAAGGCUAG | 17921 |
| 713 | P11R17_ins4_ | UCCGUUAUCAACUUGAAAAAGUGGCACCGAGUCGGUGCUUUCUGGCCUGGAUAACGGC | |
| | nick_v2 | UAUCCAGC | |
| RNACS7 | B2m1_ | ACUCACGCUGGAUAGCCUCCGUUUUAGAGCUAGAAAUAGCAAGUUAAAAUAAGGCUAG | 17922 |
| 714 | P10R17_ins4_ | UCCGUUAUCAACUUGAAAAAGUGGCACCGAGUCGGUGCUUUCUGGCCUGGAUAACGGC | |
| | nick_v2 | UAUCCAG | |
| RNACS7 | B2m1_ | ACUCACGCUGGAUAGCCUCCGUUUUAGAGCUAGAAAUAGCAAGUUAAAAUAAGGCUAG | 17923 |
| 715 | P9R17_ins4_ | UCCGUUAUCAACUUGAAAAAGUGGCACCGAGUCGGUGCUUUCUGGCCUGGAUAACGGC | |
| | nick_v2 | UAUCCA | |
| RNACS7 | B2m1_ | ACUCACGCUGGAUAGCCUCCGUUUUAGAGCUAGAAAUAGCAAGUUAAAAUAAGGCUAG | 17924 |
| 716 | P8R17_ins4_ | UCCGUUAUCAACUUGAAAAAGUGGCACCGAGUCGGUGCUUUCUGGCCUGGAUAACGGC | |
| | nick_v2 | UAUCC | |
| RNACS7 | B2m1_ | ACUCACGCUGGAUAGCCUCCGUUUUAGAGCUAGAAAUAGCAAGUUAAAAUAAGGCUAG | 17925 |
| 717 | P13R19_ins4_ | UCCGUUAUCAACUUGAAAAAGUGGCACCGAGUCGGUGCUCUUUCUGGCCUGGAUAACG | |
| | nick_v2 | GCUAUCCAGCGU | |
| RNACS7 | B2m1_ | ACUCACGCUGGAUAGCCUCCGUUUUAGAGCUAGAAAUAGCAAGUUAAAAUAAGGCUAG | 17926 |
| 718 | P12R19_ins4_ | UCCGUUAUCAACUUGAAAAAGUGGCACCGAGUCGGUGCUCUUUCUGGCCUGGAUAACG | |
| | nick_v2 | GCUAUCCAGCG | |
| RNACS7 | B2m1_ | ACUCACGCUGGAUAGCCUCCGUUUUAGAGCUAGAAAUAGCAAGUUAAAAUAAGGCUAG | 17927 |
| 719 | P11R19_ins4_ | UCCGUUAUCAACUUGAAAAAGUGGCACCGAGUCGGUGCUCUUUCUGGCCUGGAUAACG | |
| | nick_v2 | GCUAUCCAGC | |
| RNACS7 | B2m1_ | ACUCACGCUGGAUAGCCUCCGUUUUAGAGCUAGAAAUAGCAAGUUAAAAUAAGGCUAG | 17928 |
| 720 | P10R19_ins4_ | UCCGUUAUCAACUUGAAAAAGUGGCACCGAGUCGGUGCUCUUUCUGGCCUGGAUAACG | |
| | nick_v2 | GCUAUCCAG | |
| RNACS7 | B2m1_ | ACUCACGCUGGAUAGCCUCCGUUUUAGAGCUAGAAAUAGCAAGUUAAAAUAAGGCUAG | 17929 |
| 721 | P9R19_ins4_ | UCCGUUAUCAACUUGAAAAAGUGGCACCGAGUCGGUGCUCUUUCUGGCCUGGAUAACG | |
| | nick_v2 | GCUAUCCA | |
| RNACS7 | B2m1_ | ACUCACGCUGGAUAGCCUCCGUUUUAGAGCUAGAAAUAGCAAGUUAAAAUAAGGCUAG | 17930 |
| 722 | P8R19_ins4_ | UCCGUUAUCAACUUGAAAAAGUGGCACCGAGUCGGUGCUCUUUCUGGCCUGGAUAACG | |
| | nick_v2 | GCUAUCC | |
| RNACS7 | B2m1_ | ACUCACGCUGGAUAGCCUCCGUUUUAGAGCUAGAAAUAGCAAGUUAAAAUAAGGCUAG | 17931 |
| 723 | P13R21_ins4_ | UCCGUUAUCAACUUGAAAAAGUGGCACCGAGUCGGUGCUCUCUUUCUGGCCUGGAUAA | |
| | nick_v2 | CGGCUAUCCAGCGU | |
| RNACS7 | B2m1_ | ACUCACGCUGGAUAGCCUCCGUUUUAGAGCUAGAAAUAGCAAGUUAAAAUAAGGCUAG | 17932 |
| 724 | P12R21_ins4_ | UCCGUUAUCAACUUGAAAAAGUGGCACCGAGUCGGUGCUCUCUUUCUGGCCUGGAUAA | |
| | nick_v2 | CGGCUAUCCAGCG | |
| RNACS7 | B2m1_ | ACUCACGCUGGAUAGCCUCCGUUUUAGAGCUAGAAAUAGCAAGUUAAAAUAAGGCUAG | 17933 |
| 725 | P11R21_ins4_ | UCCGUUAUCAACUUGAAAAAGUGGCACCGAGUCGGUGCUCUCUUUCUGGCCUGGAUAA | |
| | nick_v2 | CGGCUAUCCAGC | |
| RNACS7 | B2m1_ | ACUCACGCUGGAUAGCCUCCGUUUUAGAGCUAGAAAUAGCAAGUUAAAAUAAGGCUAG | 17934 |
| 726 | P10R21_ins4_ | UCCGUUAUCAACUUGAAAAAGUGGCACCGAGUCGGUGCUCUCUUUCUGGCCUGGAUAA | |
| | ck_v2 | CGGCUAUCCAG | |
| RNACS7 | niB2m1_ | ACUCACGCUGGAUAGCCUCCGUUUUAGAGCUAGAAAUAGCAAGUUAAAAUAAGGCUAG | 17935 |
| 727 | P9R21_ins4_ | UCCGUUAUCAACUUGAAAAAGUGGCACCGAGUCGGUGCUCUCUUUCUGGCCUGGAUAA | |
| | nick_v2 | CGGCUAUCCA | |
| RNACS7 | B2m1_ | ACUCACGCUGGAUAGCCUCCGUUUUAGAGCUAGAAAUAGCAAGUUAAAAUAAGGCUAG | 17936 |
| 728 | P8R21_ins4_ | UCCGUUAUCAACUUGAAAAAGUGGCACCGAGUCGGUGCUCUCUUUCUGGCCUGGAUAA | |
| | nick_v2 | CGGCUAUCC | |
| RNACS7 | B2m1_ | ACUCACGCUGGAUAGCCUCCGUUUUAGAGCUAGAAAUAGCAAGUUAAAAUAAGGCUAG | 17937 |
| 729 | P13R23_ins4_ | UCCGUUAUCAACUUGAAAAAGUGGCACCGAGUCGGUGCUCUCUCUUUCUGGCCUGGAU | |
| | nick_v2 | AACGGCUAUCCAGCGU | |
| RNACS7 | B2m1_ | ACUCACGCUGGAUAGCCUCCGUUUUAGAGCUAGAAAUAGCAAGUUAAAAUAAGGCUAG | 17938 |
| 730 | P12R23_ins4_ | UCCGUUAUCAACUUGAAAAAGUGGCACCGAGUCGGUGCUCUCUCUUUCUGGCCUGGAU | |
| | nick_v2 | AACGGCUAUCCAGCG | |
| RNACS7 | B2m1_ | ACUCACGCUGGAUAGCCUCCGUUUUAGAGCUAGAAAUAGCAAGUUAAAAUAAGGCUAG | 17939 |
| 731 | P11R23_ins4_ | UCCGUUAUCAACUUGAAAAAGUGGCACCGAGUCGGUGCUCUCUCUUUCUGGCCUGGAU | |
| | nick_v2 | AACGGCUAUCCAGC | |
| RNACS7 | B2m1_ | ACUCACGCUGGAUAGCCUCCGUUUUAGAGCUAGAAAUAGCAAGUUAAAAUAAGGCUAG | 17940 |
| 732 | P10R23_ins4_ | UCCGUUAUCAACUUGAAAAAGUGGCACCGAGUCGGUGCUCUCUCUUUCUGGCCUGGAU | |
| | nick_v2 | AACGGCUAUCCAG | |
| RNACS7 | B2m1_ | ACUCACGCUGGAUAGCCUCCGUUUUAGAGCUAGAAAUAGCAAGUUAAAAUAAGGCUAG | 17941 |
| 733 | P9R23_ins4_ | UCCGUUAUCAACUUGAAAAAGUGGCACCGAGUCGGUGCUCUCUCUUUCUGGCCUGGAU | |
| | nick_v2 | AACGGCUAUCCA | |
| RNACS7 | B2m1_ | ACUCACGCUGGAUAGCCUCCGUUUUAGAGCUAGAAAUAGCAAGUUAAAAUAAGGCUAG | 17942 |
| 734 | P8R23_ins4_ | UCCGUUAUCAACUUGAAAAAGUGGCACCGAGUCGGUGCUCUCUCUUUCUGGCCUGGAU | |
| | nick_v2 | AACGGCUAUCC | |
| RNACS7 | B2m1_ | ACUCACGCUGGAUAGCCUCCGUUUUAGAGCUAGAAAUAGCAAGUUAAAAUAAGGCUAG | 17943 |
| 735 | P13R12_ins5_ | UCCGUUAUCAACUUGAAAAAGUGGCACCGAGUCGGUGCGCCUGGAUGAUGGGCUAUCC | |
| | nick_v1 | AGCGU | |
| RNACS7 | B2m1_ | ACUCACGCUGGAUAGCCUCCGUUUUAGAGCUAGAAAUAGCAAGUUAAAAUAAGGCUAG | 17944 |
| 736 | P12R12_ins5_ | UCCGUUAUCAACUUGAAAAAGUGGCACCGAGUCGGUGCGCCUGGAUGAUGGGCUAUCC | |
| | nick_v1 | AGCG | |
| RNACS7 | B2m1_ | ACUCACGCUGGAUAGCCUCCGUUUUAGAGCUAGAAAUAGCAAGUUAAAAUAAGGCUAG | 17945 |
| 737 | P11R12_ins5_ | UCCGUUAUCAACUUGAAAAAGUGGCACCGAGUCGGUGCGCCUGGAUGAUGGGCUAUCC | |
| | nick_v1 | AGC | |
| RNACS7 | B2m1_ | ACUCACGCUGGAUAGCCUCCGUUUUAGAGCUAGAAAUAGCAAGUUAAAAUAAGGCUAG | 17946 |
| 738 | P10R12_ins5_ | UCCGUUAUCAACUUGAAAAAGUGGCACCGAGUCGGUGCGCCUGGAUGAUGGGCUAUCC | |
| | nick_v1 | AG | |
| RNACS7 | B2m1_ | ACUCACGCUGGAUAGCCUCCGUUUUAGAGCUAGAAAUAGCAAGUUAAAAUAAGGCUAG | 17947 |
| 739 | P9R12_ins5_ | UCCGUUAUCAACUUGAAAAAGUGGCACCGAGUCGGUGCGCCUGGAUGAUGGGCUAUCC | |
| | nick_v1 | A | |
| RNACS7 | B2m1_ | ACUCACGCUGGAUAGCCUCCGUUUUAGAGCUAGAAAUAGCAAGUUAAAAUAAGGCUAG | 17948 |
| 740 | 1P8R2_ins5_ | UCCGUUAUCAACUUGAAAAAGUGGCACCGAGUCGGUGCGCCUGGAUGAUGGGCUAUCC | |
| | nick_v1 | | |
| RNACS7 | B2m1_ | ACUCACGCUGGAUAGCCUCCGUUUUAGAGCUAGAAAUAGCAAGUUAAAAUAAGGCUAG | 17949 |
| 741 | RP1314_ins5_ | UCCGUUAUCAACUUGAAAAAGUGGCACCGAGUCGGUGCUGGCCUGGAUGAUGGGCUAU | |
| | nick_v1 | CCAGCGU | |
| RNACS7 | B2m1_ | ACUCACGCUGGAUAGCCUCCGUUUUAGAGCUAGAAAUAGCAAGUUAAAAUAAGGCUAG | 17950 |
| 742 | P12R14_ins5_ | UCCGUUAUCAACUUGAAAAAGUGGCACCGAGUCGGUGCUGGCCUGGAUGAUGGGCUAU | |
| | nick_v1 | CCAGCG | |
| RNACS7 | B2m1_ | ACUCACGCUGGAUAGCCUCCGUUUUAGAGCUAGAAAUAGCAAGUUAAAAUAAGGCUAG | 17951 |
| 743 | P11R14_ins5_ | UCCGUUAUCAACUUGAAAAAGUGGCACCGAGUCGGUGCUGGCCUGGAUGAUGGGCUAU | |
| | nick_v1 | CCAGC | |
| RNACS7 | B2m1_ | ACUCACGCUGGAUAGCCUCCGUUUUAGAGCUAGAAAUAGCAAGUUAAAAUAAGGCUAG | 17952 |
| 744 | P10R14_ins5_ | UCCGUUAUCAACUUGAAAAAGUGGCACCGAGUCGGUGCUGGCCUGGAUGAUGGGCUAU | |
| | nick_v1 | CCAG | |
| RNACS7 | B2m1_ | ACUCACGCUGGAUAGCCUCCGUUUUAGAGCUAGAAAUAGCAAGUUAAAAUAAGGCUAG | 17953 |
| 745 | P9R14_ins5_ | UCCGUUAUCAACUUGAAAAAGUGGCACCGAGUCGGUGCUGGCCUGGAUGAUGGGCUAU | |
| | nick_v1 | CCA | |
| RNACS7 | B2m1_ | ACUCACGCUGGAUAGCCUCCGUUUUAGAGCUAGAAAUAGCAAGUUAAAAUAAGGCUAG | 17954 |
| 746 | P8R14_ins5_ | UCCGUUAUCAACUUGAAAAAGUGGCACCGAGUCGGUGCUGGCCUGGAUGAUGGGCUAU | |
| | nick_v1 | CC | |
| RNACS7 | B2m1_ | ACUCACGCUGGAUAGCCUCCGUUUUAGAGCUAGAAAUAGCAAGUUAAAAUAAGGCUAG | 17955 |
| 747 | P13R16_ins5_ | UCCGUUAUCAACUUGAAAAAGUGGCACCGAGUCGGUGCUCUGGCCUGGAUGAUGGGCU | |
| | nick_v1 | AUCCAGCGU | |
| RNACS7 | B2m1_ | ACUCACGCUGGAUAGCCUCCGUUUUAGAGCUAGAAAUAGCAAGUUAAAAUAAGGCUAG | 17956 |
| 748 | P12R16_ins5_ | UCCGUUAUCAACUUGAAAAAGUGGCACCGAGUCGGUGCUCUGGCCUGGAUGAUGGGCU | |
| | nick_v1 | AUCCAGCG | |
| RNACS7 | B2m1_ | ACUCACGCUGGAUAGCCUCCGUUUUAGAGCUAGAAAUAGCAAGUUAAAAUAAGGCUAG | 17957 |
| 749 | P11R16_ins5_ | UCCGUUAUCAACUUGAAAAAGUGGCACCGAGUCGGUGCUCUGGCCUGGAUGAUGGGCU | |
| | nick_v1 | AUCCAGC | |
| RNACS7 | B2m1_ | ACUCACGCUGGAUAGCCUCCGUUUUAGAGCUAGAAAUAGCAAGUUAAAAUAAGGCUAG | 17958 |
| 750 | P10R16_ins5_ | UCCGUUAUCAACUUGAAAAAGUGGCACCGAGUCGGUGCUCUGGCCUGGAUGAUGGGCU | |
| | nick_v1 | AUCCAG | |
| RNACS7 | B2m1_ | ACUCACGCUGGAUAGCCUCCGUUUUAGAGCUAGAAAUAGCAAGUUAAAAUAAGGCUAG | 17959 |
| 751 | 1P9R6_ins5_ | UCCGUUAUCAACUUGAAAAAGUGGCACCGAGUCGGUGCUCUGGCCUGGAUGAUGGGCU | |
| | nick_v1 | AUCCA | |
| RNACS7 | B2m1_ | ACUCACGCUGGAUAGCCUCCGUUUUAGAGCUAGAAAUAGCAAGUUAAAAUAAGGCUAG | 17960 |
| 752 | P8R16_ins5_ | UCCGUUAUCAACUUGAAAAAGUGGCACCGAGUCGGUGCUCUGGCCUGGAUGAUGGGCU | |
| | nick_v1 | AUCC | |
| RNACS7 | B2m1_ | ACUCACGCUGGAUAGCCUCCGUUUUAGAGCUAGAAAUAGCAAGUUAAAAUAAGGCUAG | 17961 |
| 753 | P13R18_ins5_ | UCCGUUAUCAACUUGAAAAAGUGGCACCGAGUCGGUGCUUUCUGGCCUGGAUGAUGGG | |
| | nick_v1 | CUAUCCAGCGU | |
| RNACS7 | B2m1_ | ACUCACGCUGGAUAGCCUCCGUUUUAGAGCUAGAAAUAGCAAGUUAAAAUAAGGCUAG | 17962 |
| 754 | P12R18_ins5_ | UCCGUUAUCAACUUGAAAAAGUGGCACCGAGUCGGUGCUUUCUGGCCUGGAUGAUGGG | |
| | nick_v1 | CUAUCCAGCG | |
| RNACS7 | B2m1_ | ACUCACGCUGGAUAGCCUCCGUUUUAGAGCUAGAAAUAGCAAGUUAAAAUAAGGCUAG | 17963 |
| 755 | P11R18_ins5_ | UCCGUUAUCAACUUGAAAAAGUGGCACCGAGUCGGUGCUUUCUGGCCUGGAUGAUGGG | |
| | nick_v1 | CUAUCCAGC | |
| RNACS7 | B2m1_ | ACUCACGCUGGAUAGCCUCCGUUUUAGAGCUAGAAAUAGCAAGUUAAAAUAAGGCUAG | 17964 |
| 756 | P10R18_ins5_ | UCCGUUAUCAACUUGAAAAAGUGGCACCGAGUCGGUGCUUUCUGGCCUGGAUGAUGGG | |
| | nick_v1 | CUAUCCAG | |
| RNACS7 | B2m1_ | ACUCACGCUGGAUAGCCUCCGUUUUAGAGCUAGAAAUAGCAAGUUAAAAUAAGGCUAG | 17965 |
| 757 | P9R18_ins5_ | UCCGUUAUCAACUUGAAAAAGUGGCACCGAGUCGGUGCUUUCUGGCCUGGAUGAUGGG | |
| | nick_v1 | CUAUCCA | |
| RNACS7 | B2m1_ | ACUCACGCUGGAUAGCCUCCGUUUUAGAGCUAGAAAUAGCAAGUUAAAAUAAGGCUAG | 17966 |
| 758 | P8R18_ins5_ | UCCGUUAUCAACUUGAAAAAGUGGCACCGAGUCGGUGCUUUCUGGCCUGGAUGAUGGG | |
| | nick_v1 | CUAUCC | |
| RNACS7 | B2m1_ | ACUCACGCUGGAUAGCCUCCGUUUUAGAGCUAGAAAUAGCAAGUUAAAAUAAGGCUAG | 17967 |
| 759 | P13R20_ins5_ | UCCGUUAUCAACUUGAAAAAGUGGCACCGAGUCGGUGCUCUUUCUGGCCUGGAUGAUG | |
| | nick_v1 | GGCUAUCCAGCGU | |
| RNACS7 | B2m1_ | ACUCACGCUGGAUAGCCUCCGUUUUAGAGCUAGAAAUAGCAAGUUAAAAUAAGGCUAG | 17968 |
| 760 | P12R20_ins5_ | UCCGUUAUCAACUUGAAAAAGUGGCACCGAGUCGGUGCUCUUUCUGGCCUGGAUGAUG | |
| | nick_v1 | GGCUAUCCAGCG | |
| RNACS7 | B2m1_ | ACUCACGCUGGAUAGCCUCCGUUUUAGAGCUAGAAAUAGCAAGUUAAAAUAAGGCUAG | 17969 |
| 761 | P11R20_ins5_ | UCCGUUAUCAACUUGAAAAAGUGGCACCGAGUCGGUGCUCUUUCUGGCCUGGAUGAUG | |
| | nick_v1 | GGCUAUCCAGC | |
| RNACS7 | B2m1_ | ACUCACGCUGGAUAGCCUCCGUUUUAGAGCUAGAAAUAGCAAGUUAAAAUAAGGCUAG | 17970 |
| 762 | P10R20_ins5_ | UCCGUUAUCAACUUGAAAAAGUGGCACCGAGUCGGUGCUCUUUCUGGCCUGGAUGAUG | |
| | nick_v1 | GGCUAUCCAG | |
| RNACS7 | B2m1_ | ACUCACGCUGGAUAGCCUCCGUUUUAGAGCUAGAAAUAGCAAGUUAAAAUAAGGCUAG | 17971 |
| 763 | P9R20_ins5_ | UCCGUUAUCAACUUGAAAAAGUGGCACCGAGUCGGUGCUCUUUCUGGCCUGGAUGAUG | |
| | nick_v1 | GGCUAUCCA | |
| RNACS7 | B2m1_ | ACUCACGCUGGAUAGCCUCCGUUUUAGAGCUAGAAAUAGCAAGUUAAAAUAAGGCUAG | 17972 |
| 764 | P8R20_ins5_ | UCCGUUAUCAACUUGAAAAAGUGGCACCGAGUCGGUGCUCUUUCUGGCCUGGAUGAUG | |
| | nick_v1 | GGCUAUCC | |
| RNACS7 | B2m1_ | ACUCACGCUGGAUAGCCUCCGUUUUAGAGCUAGAAAUAGCAAGUUAAAAUAAGGCUAG | 17973 |
| 765 | P13R22_ins5_ | UCCGUUAUCAACUUGAAAAAGUGGCACCGAGUCGGUGCUCUCUUUCUGGCCUGGAUGA | |
| | nick_v1 | UGGGCUAUCCAGCGU | |
| RNACS7 | B2m1_ | ACUCACGCUGGAUAGCCUCCGUUUUAGAGCUAGAAAUAGCAAGUUAAAAUAAGGCUAG | 17974 |
| 766 | P12R22_ins5_ | UCCGUUAUCAACUUGAAAAAGUGGCACCGAGUCGGUGCUCUCUUUCUGGCCUGGAUGA | |
| | cnik_v1 | UGGGCUAUCCAGCG | |
| RNACS7 | B2m1_ | ACUCACGCUGGAUAGCCUCCGUUUUAGAGCUAGAAAUAGCAAGUUAAAAUAAGGCUAG | 17975 |
| 767 | P11R22_ins5_ | UCCGUUAUCAACUUGAAAAAGUGGCACCGAGUCGGUGCUCUCUUUCUGGCCUGGAUGA | |
| | nick_v1 | UGGGCUAUCCAGC | |
| RNACS7 | B2m1_ | ACUCACGCUGGAUAGCCUCCGUUUUAGAGCUAGAAAUAGCAAGUUAAAAUAAGGCUAG | 17976 |
| 768 | P10R22_ins5_ | UCCGUUAUCAACUUGAAAAAGUGGCACCGAGUCGGUGCUCUCUUUCUGGCCUGGAUGA | |
| | nick_v1 | UGGGCUAUCCAG | |
| RNACS7 | B2m1_ | ACUCACGCUGGAUAGCCUCCGUUUUAGAGCUAGAAAUAGCAAGUUAAAAUAAGGCUAG | 17977 |
| 769 | P9R22_ins5_ | UCCGUUAUCAACUUGAAAAAGUGGCACCGAGUCGGUGCUCUCUUUCUGGCCUGGAUGA | |
| | nick_v1 | UGGGCUAUCCA | |
| RNACS7 | B2m1_ | ACUCACGCUGGAUAGCCUCCGUUUUAGAGCUAGAAAUAGCAAGUUAAAAUAAGGCUAG | 17978 |
| 770 | P8R22_ins5_ | UCCGUUAUCAACUUGAAAAAGUGGCACCGAGUCGGUGCUCUCUUUCUGGCCUGGAUGA | |
| | nick_v1 | UGGGCUAUCC | |
| RNACS7 | B2m1_ | ACUCACGCUGGAUAGCCUCCGUUUUAGAGCUAGAAAUAGCAAGUUAAAAUAAGGCUAG | 17979 |
| 771 | P13R24_ins5_ | UCCGUUAUCAACUUGAAAAAGUGGCACCGAGUCGGUGCUCUCUCUUUCUGGCCUGGAU | |
| | nick_v1 | GAUGGGCUAUCCAGCGU | |
| RNACS7 | B2m1_ | ACUCACGCUGGAUAGCCUCCGUUUUAGAGCUAGAAAUAGCAAGUUAAAAUAAGGCUAG | 17980 |
| 772 | P12R24_ins5_ | UCCGUUAUCAACUUGAAAAAGUGGCACCGAGUCGGUGCUCUCUCUUUCUGGCCUGGAU | |
| | nick_v1 | GAUGGGCUAUCCAGCG | |
| RNACS7 | B2m1_ | ACUCACGCUGGAUAGCCUCCGUUUUAGAGCUAGAAAUAGCAAGUUAAAAUAAGGCUAG | 17981 |
| 773 | P11R24_ins5_ | UCCGUUAUCAACUUGAAAAAGUGGCACCGAGUCGGUGCUCUCUCUUUCUGGCCUGGAU | |
| | nick_v1 | GAUGGGCUAUCCAGC | |
| RNACS7 | B2m1_ | ACUCACGCUGGAUAGCCUCCGUUUUAGAGCUAGAAAUAGCAAGUUAAAAUAAGGCUAG | 17982 |
| 774 | RP1024_ins5_ | UCCGUUAUCAACUUGAAAAAGUGGCACCGAGUCGGUGCUCUCUCUUUCUGGCCUGGAU | |
| | nick_v1 | GAUGGGCUAUCCAG | |
| RNACS7 | B2m1_ | ACUCACGCUGGAUAGCCUCCGUUUUAGAGCUAGAAAUAGCAAGUUAAAAUAAGGCUAG | 17983 |
| 775 | P9R24_ins5_ | UCCGUUAUCAACUUGAAAAAGUGGCACCGAGUCGGUGCUCUCUCUUUCUGGCCUGGAU | |
| | nick_v1 | GAUGGGCUAUCCA | |
| RNACS7 | B2m1_ | ACUCACGCUGGAUAGCCUCCGUUUUAGAGCUAGAAAUAGCAAGUUAAAAUAAGGCUAG | 17984 |
| 776 | P8R24_ins5_ | UCCGUUAUCAACUUGAAAAAGUGGCACCGAGUCGGUGCUCUCUCUUUCUGGCCUGGAU | |
| | nick_v1 | GAUGGGCUAUCC | |
| RNACS7 | B2m1_ | ACUCACGCUGGAUAGCCUCCGUUUUAGAGCUAGAAAUAGCAAGUUAAAAUAAGGCUAG | 17985 |
| 777 | P13R12_ins5_ | UCCGUUAUCAACUUGAAAAAGUGGCACCGAGUCGGUGCGCCUGGAUAACCGGCUAUCC | |
| | nick_v2 | AGCGU | |
| RNACS7 | B2m1_ | ACUCACGCUGGAUAGCCUCCGUUUUAGAGCUAGAAAUAGCAAGUUAAAAUAAGGCUAG | 17986 |
| 778 | P12R12_ins5_ | UCCGUUAUCAACUUGAAAAAGUGGCACCGAGUCGGUGCGCCUGGAUAACCGGCUAUCC | |
| | nick_v2 | AGCG | |
| RNACS7 | B2m1_ | ACUCACGCUGGAUAGCCUCCGUUUUAGAGCUAGAAAUAGCAAGUUAAAAUAAGGCUAG | 17987 |
| 779 | P11R12_ins5_ | UCCGUUAUCAACUUGAAAAAGUGGCACCGAGUCGGUGCGCCUGGAUAACCGGCUAUCC | |
| | nick_v2 | AGC | |
| RNACS7 | B2m1_ | ACUCACGCUGGAUAGCCUCCGUUUUAGAGCUAGAAAUAGCAAGUUAAAAUAAGGCUAG | 17988 |
| 780 | P10R12_ins5_ | UCCGUUAUCAACUUGAAAAAGUGGCACCGAGUCGGUGCGCCUGGAUAACCGGCUAUCC | |
| | nick_v2 | AG | |
| RNACS7 | B2m1_ | ACUCACGCUGGAUAGCCUCCGUUUUAGAGCUAGAAAUAGCAAGUUAAAAUAAGGCUAG | 17989 |
| 781 | P9R12_ins5_ | UCCGUUAUCAACUUGAAAAAGUGGCACCGAGUCGGUGCGCCUGGAUAACCGGCUAUCC | |
| | nick_v2 | A | |
| RNACS7 | B2m1_ | ACUCACGCUGGAUAGCCUCCGUUUUAGAGCUAGAAAUAGCAAGUUAAAAUAAGGCUAG | 17990 |
| 782 | P8R12_ins5_ | UCCGUUAUCAACUUGAAAAAGUGGCACCGAGUCGGUGCGCCUGGAUAACCGGCUAUCC | |
| | nick_v2 | | |
| RNACS7 | B2m1_ | ACUCACGCUGGAUAGCCUCCGUUUUAGAGCUAGAAAUAGCAAGUUAAAAUAAGGCUAG | 17991 |
| 783 | P13R14_ins5_ | UCCGUUAUCAACUUGAAAAAGUGGCACCGAGUCGGUGCUGGCCUGGAUAACCGGCUAU | |
| | nick_v2 | CCAGCGU | |
| RNACS7 | B2m1_ | ACUCACGCUGGAUAGCCUCCGUUUUAGAGCUAGAAAUAGCAAGUUAAAAUAAGGCUAG | 17992 |
| 784 | P12R14_ins5_ | UCCGUUAUCAACUUGAAAAAGUGGCACCGAGUCGGUGCUGGCCUGGAUAACCGGCUAU | |
| | nick_v2 | CCAGCG | |
| RNACS7 | B2m1_ | ACUCACGCUGGAUAGCCUCCGUUUUAGAGCUAGAAAUAGCAAGUUAAAAUAAGGCUAG | 17993 |
| 785 | P11R14_ins5_ | UCCGUUAUCAACUUGAAAAAGUGGCACCGAGUCGGUGCUGGCCUGGAUAACCGGCUAU | |
| | nick_v2 | CCAGC | |
| RNACS7 | B2m1_ | ACUCACGCUGGAUAGCCUCCGUUUUAGAGCUAGAAAUAGCAAGUUAAAAUAAGGCUAG | 17994 |
| 786 | P10R14_ins5_ | UCCGUUAUCAACUUGAAAAAGUGGCACCGAGUCGGUGCUGGCCUGGAUAACCGGCUAU | |
| | nick_v2 | CCAG | |
| RNACS7 | B2m1_ | ACUCACGCUGGAUAGCCUCCGUUUUAGAGCUAGAAAUAGCAAGUUAAAAUAAGGCUAG | 17995 |
| 787 | P9R14_ins5_ | UCCGUUAUCAACUUGAAAAAGUGGCACCGAGUCGGUGCUGGCCUGGAUAACCGGCUAU | |
| | nick_v2 | CCA | |
| RNACS7 | B2m1_ | ACUCACGCUGGAUAGCCUCCGUUUUAGAGCUAGAAAUAGCAAGUUAAAAUAAGGCUAG | 17996 |
| 788 | P8R14_ins5_ | UCCGUUAUCAACUUGAAAAAGUGGCACCGAGUCGGUGCUGGCCUGGAUAACCGGCUAU | |
| | nick_v2 | CC | |
| RNACS7 | B2m1_ | ACUCACGCUGGAUAGCCUCCGUUUUAGAGCUAGAAAUAGCAAGUUAAAAUAAGGCUAG | 17997 |
| 789 | P13R16_ins5_ | UCCGUUAUCAACUUGAAAAAGUGGCACCGAGUCGGUGCUCUGGCCUGGAUAACCGGCU | |
| | nick_v2 | AUCCAGCGU | |
| RNACS7 | B2m1_ | ACUCACGCUGGAUAGCCUCCGUUUUAGAGCUAGAAAUAGCAAGUUAAAAUAAGGCUAG | 17998 |
| 790 | P12R16_ins5_ | UCCGUUAUCAACUUGAAAAAGUGGCACCGAGUCGGUGCUCUGGCCUGGAUAACCGGCU | |
| | nick_v2 | AUCCAGCG | |
| RNACS7 | B2m1_ | ACUCACGCUGGAUAGCCUCCGUUUUAGAGCUAGAAAUAGCAAGUUAAAAUAAGGCUAG | 17999 |
| 791 | P11R16_ins5_ | UCCGUUAUCAACUUGAAAAAGUGGCACCGAGUCGGUGCUCUGGCCUGGAUAACCGGCU | |
| | nick_v2 | AUCCAGC | |
| RNACS7 | B2m1_ | ACUCACGCUGGAUAGCCUCCGUUUUAGAGCUAGAAAUAGCAAGUUAAAAUAAGGCUAG | 18000 |
| 792 | P10R16_ins5_ | UCCGUUAUCAACUUGAAAAAGUGGCACCGAGUCGGUGCUCUGGCCUGGAUAACCGGCU | |
| | nick_v2 | AUCCAG | |
| RNACS7 | B2m1_ | ACUCACGCUGGAUAGCCUCCGUUUUAGAGCUAGAAAUAGCAAGUUAAAAUAAGGCUAG | 18001 |
| 793 | P9R16_ins5_ | UCCGUUAUCAACUUGAAAAAGUGGCACCGAGUCGGUGCUCUGGCCUGGAUAACCGGCU | |
| | nick_v2 | AUCCA | |
| RNACS7 | B2m1_ | ACUCACGCUGGAUAGCCUCCGUUUUAGAGCUAGAAAUAGCAAGUUAAAAUAAGGCUAG | 18002 |
| 794 | P8R16_ins5_ | UCCGUUAUCAACUUGAAAAAGUGGCACCGAGUCGGUGCUCUGGCCUGGAUAACCGGCU | |
| | nick_v2 | AUCC | |
| RNACS7 | B2m1_ | ACUCACGCUGGAUAGCCUCCGUUUUAGAGCUAGAAAUAGCAAGUUAAAAUAAGGCUAG | 18003 |
| 795 | P13R18_ins5_ | UCCGUUAUCAACUUGAAAAAGUGGCACCGAGUCGGUGCUUUCUGGCCUGGAUAACCGG | |
| | nick_v2 | CUAUCCAGCGU | |
| RNACS7 | B2m1_ | ACUCACGCUGGAUAGCCUCCGUUUUAGAGCUAGAAAUAGCAAGUUAAAAUAAGGCUAG | 18004 |
| 796 | P12R18_ins5_ | UCCGUUAUCAACUUGAAAAAGUGGCACCGAGUCGGUGCUUUCUGGCCUGGAUAACCGG | |
| | nick_v2 | CUAUCCAGCG | |
| RNACS7 | B2m1_ | ACUCACGCUGGAUAGCCUCCGUUUUAGAGCUAGAAAUAGCAAGUUAAAAUAAGGCUAG | 18005 |
| 797 | P11R18_ins5_ | UCCGUUAUCAACUUGAAAAAGUGGCACCGAGUCGGUGCUUUCUGGCCUGGAUAACCGG | |
| | nick_v2 | CUAUCCAGC | |
| RNACS7 | B2m1_ | ACUCACGCUGGAUAGCCUCCGUUUUAGAGCUAGAAAUAGCAAGUUAAAAUAAGGCUAG | 18006 |
| 798 | P10R18_ins5_ | UCCGUUAUCAACUUGAAAAAGUGGCACCGAGUCGGUGCUUUCUGGCCUGGAUAACCGG | |
| | nick_v2 | CUAUCCAG | |
| RNACS7 | B2m1_ | ACUCACGCUGGAUAGCCUCCGUUUUAGAGCUAGAAAUAGCAAGUUAAAAUAAGGCUAG | 18007 |
| 799 | P9R18_ins5_ | UCCGUUAUCAACUUGAAAAAGUGGCACCGAGUCGGUGCUUUCUGGCCUGGAUAACCGG | |
| | nick_v2 | CUAUCCA | |
| RNACS7 | B2m1_ | ACUCACGCUGGAUAGCCUCCGUUUUAGAGCUAGAAAUAGCAAGUUAAAAUAAGGCUAG | 18008 |
| 800 | P8R18_ins5_ | UCCGUUAUCAACUUGAAAAAGUGGCACCGAGUCGGUGCUUUCUGGCCUGGAUAACCGG | |
| | nick_v2 | CUAUCC | |
| RNACS7 | B2m1_ | ACUCACGCUGGAUAGCCUCCGUUUUAGAGCUAGAAAUAGCAAGUUAAAAUAAGGCUAG | 18009 |
| 801 | P13R20_ins5_ | UCCGUUAUCAACUUGAAAAAGUGGCACCGAGUCGGUGCUCUUUCUGGCCUGGAUAACC | |
| | nick_v2 | GGCUAUCCAGCGU | |
| RNACS7 | B2m1_ | ACUCACGCUGGAUAGCCUCCGUUUUAGAGCUAGAAAUAGCAAGUUAAAAUAAGGCUAG | 18010 |
| 802 | P12R20_ins5_ | UCCGUUAUCAACUUGAAAAAGUGGCACCGAGUCGGUGCUCUUUCUGGCCUGGAUAACC | |
| | nick_v2 | GGCUAUCCAGCG | |
| RNACS7 | B2m1_ | ACUCACGCUGGAUAGCCUCCGUUUUAGAGCUAGAAAUAGCAAGUUAAAAUAAGGCUAG | 18011 |
| 803 | P11R20_ins5_ | UCCGUUAUCAACUUGAAAAAGUGGCACCGAGUCGGUGCUCUUUCUGGCCUGGAUAACC | |
| | nick_v2 | GGCUAUCCAGC | |
| RNACS7 | B2m1_ | ACUCACGCUGGAUAGCCUCCGUUUUAGAGCUAGAAAUAGCAAGUUAAAAUAAGGCUAG | 18012 |
| 804 | P10R20_ins5_ | UCCGUUAUCAACUUGAAAAAGUGGCACCGAGUCGGUGCUCUUUCUGGCCUGGAUAACC | |
| | nick_v2 | GGCUAUCCAG | |
| RNACS7 | B2m1_ | ACUCACGCUGGAUAGCCUCCGUUUUAGAGCUAGAAAUAGCAAGUUAAAAUAAGGCUAG | 18013 |
| 805 | P9R20_ins5_ | UCCGUUAUCAACUUGAAAAAGUGGCACCGAGUCGGUGCUCUUUCUGGCCUGGAUAACC | |
| | nick_v2 | GGCUAUCCA | |
| RNACS7 | B2m1_ | ACUCACGCUGGAUAGCCUCCGUUUUAGAGCUAGAAAUAGCAAGUUAAAAUAAGGCUAG | 18014 |
| 806 | P8R20_ins5_ | UCCGUUAUCAACUUGAAAAAGUGGCACCGAGUCGGUGCUCUUUCUGGCCUGGAUAACC | |
| | nick_v2 | GGCUAUCC | |
| RNACS7 | B2m1_ | ACUCACGCUGGAUAGCCUCCGUUUUAGAGCUAGAAAUAGCAAGUUAAAAUAAGGCUAG | 18015 |
| 807 | P13R22_ins5_ | UCCGUUAUCAACUUGAAAAAGUGGCACCGAGUCGGUGCUCUCUUUCUGGCCUGGAUAA | |
| | nick_v2 | CCGGCUAUCCAGCGU | |
| RNACS7 | B2m1_ | ACUCACGCUGGAUAGCCUCCGUUUUAGAGCUAGAAAUAGCAAGUUAAAAUAAGGCUAG | 18016 |
| 808 | P12R22_ins5_ | UCCGUUAUCAACUUGAAAAAGUGGCACCGAGUCGGUGCUCUCUUUCUGGCCUGGAUAA | |
| | nick_v2 | CCGGCUAUCCAGCG | |
| RNACS7 | B2m1_ | ACUCACGCUGGAUAGCCUCCGUUUUAGAGCUAGAAAUAGCAAGUUAAAAUAAGGCUAG | 18017 |
| 809 | P11R22_ins5_ | UCCGUUAUCAACUUGAAAAAGUGGCACCGAGUCGGUGCUCUCUUUCUGGCCUGGAUAA | |
| | nick_v2 | CCGGCUAUCCAGC | |
| RNACS7 | B2m1_ | ACUCACGCUGGAUAGCCUCCGUUUUAGAGCUAGAAAUAGCAAGUUAAAAUAAGGCUAG | 18018 |
| 810 | P10R22_ins5_ | UCCGUUAUCAACUUGAAAAAGUGGCACCGAGUCGGUGCUCUCUUUCUGGCCUGGAUAA | |
| | nick_v2 | CCGGCUAUCCAG | |
| RNACS7 | B2m1_ | ACUCACGCUGGAUAGCCUCCGUUUUAGAGCUAGAAAUAGCAAGUUAAAAUAAGGCUAG | 18019 |
| 811 | P9R22_ins5_ | UCCGUUAUCAACUUGAAAAAGUGGCACCGAGUCGGUGCUCUCUUUCUGGCCUGGAUAA | |
| | nick_v2 | CCGGCUAUCCA | |
| RNACS7 | B2m1_ | ACUCACGCUGGAUAGCCUCCGUUUUAGAGCUAGAAAUAGCAAGUUAAAAUAAGGCUAG | 18020 |
| 812 | P8R22_ins5_ | UCCGUUAUCAACUUGAAAAAGUGGCACCGAGUCGGUGCUCUCUUUCUGGCCUGGAUAA | |
| | nick_v2 | CCGGCUAUCC | |
| RNACS7 | B2m1_ | ACUCACGCUGGAUAGCCUCCGUUUUAGAGCUAGAAAUAGCAAGUUAAAAUAAGGCUAG | 18021 |
| 813 | P13R24_ins5_ | UCCGUUAUCAACUUGAAAAAGUGGCACCGAGUCGGUGCUCUCUCUUUCUGGCCUGGAU | |
| | nick_v2 | AACCGGCUAUCCAGCGU | |
| RNACS7 | B2m1_ | ACUCACGCUGGAUAGCCUCCGUUUUAGAGCUAGAAAUAGCAAGUUAAAAUAAGGCUAG | 18022 |
| 814 | P12R24_ins5_ | UCCGUUAUCAACUUGAAAAAGUGGCACCGAGUCGGUGCUCUCUCUUUCUGGCCUGGAU | |
| | nick_v2 | AACCGGCUAUCCAGCG | |
| RNACS7 | B2m1_ | ACUCACGCUGGAUAGCCUCCGUUUUAGAGCUAGAAAUAGCAAGUUAAAAUAAGGCUAG | 18023 |
| 815 | P11R24_ins5_ | UCCGUUAUCAACUUGAAAAAGUGGCACCGAGUCGGUGCUCUCUCUUUCUGGCCUGGAU | |
| | nick_v2 | AACCGGCUAUCCAGC | |
| RNACS7 | B2m1_ | ACUCACGCUGGAUAGCCUCCGUUUUAGAGCUAGAAAUAGCAAGUUAAAAUAAGGCUAG | 18024 |
| 816 | P10R24_ins5_ | UCCGUUAUCAACUUGAAAAAGUGGCACCGAGUCGGUGCUCUCUCUUUCUGGCCUGGAU | |
| | nick_v2 | AACCGGCUAUCCAG | |
| RNACS7 | B2m1_ | ACUCACGCUGGAUAGCCUCCGUUUUAGAGCUAGAAAUAGCAAGUUAAAAUAAGGCUAG | 18025 |
| 817 | P9R24_ins5_ | UCCGUUAUCAACUUGAAAAAGUGGCACCGAGUCGGUGCUCUCUCUUUCUGGCCUGGAU | |
| | nick_v2 | AACCGGCUAUCCA | |
| RNACS7 | B2m1_ | ACUCACGCUGGAUAGCCUCCGUUUUAGAGCUAGAAAUAGCAAGUUAAAAUAAGGCUAG | 18026 |
| 818 | P8R24_ins5_ | UCCGUUAUCAACUUGAAAAAGUGGCACCGAGUCGGUGCUCUCUCUUUCUGGCCUGGAU | |
| | nick_v2 | AACCGGCUAUCC | |
| RNACS7 | B2m2_ | GAGUAGCGCGAGCACAGCUAGUUUUAGAGCUAGAAAUAGCAAGUUAAAAUAAGGCUAG | 18027 |
| 819 | P13R11_ins4_ | UCCGUUAUCAACUUGAAAAAGUGGCACCGAGUCGGUGCGCCUUAGUGAUCUGUGCUCG | |
| | nick_v1 | CGCU | |
| RNACS7 | B2m2_ | GAGUAGCGCGAGCACAGCUAGUUUUAGAGCUAGAAAUAGCAAGUUAAAAUAAGGCUAG | 18028 |
| 820 | P12R11_ins4_ | UCCGUUAUCAACUUGAAAAAGUGGCACCGAGUCGGUGCGCCUUAGUGAUCUGUGCUCG | |
| | nick_v1 | CGC | |
| RNACS7 | B2m2_ | GAGUAGCGCGAGCACAGCUAGUUUUAGAGCUAGAAAUAGCAAGUUAAAAUAAGGCUAG | 18029 |
| 821 | P11R11_ins4_ | UCCGUUAUCAACUUGAAAAAGUGGCACCGAGUCGGUGCGCCUUAGUGAUCUGUGCUCG | |
| | nick_v1 | CG | |
| RNACS7 | B2m2_ | GAGUAGCGCGAGCACAGCUAGUUUUAGAGCUAGAAAUAGCAAGUUAAAAUAAGGCUAG | 18030 |
| 822 | P10R11_ins4_ | UCCGUUAUCAACUUGAAAAAGUGGCACCGAGUCGGUGCGCCUUAGUGAUCUGUGCUCG | |
| | nick_v1 | C | |
| RNACS7 | B2m2_ | GAGUAGCGCGAGCACAGCUAGUUUUAGAGCUAGAAAUAGCAAGUUAAAAUAAGGCUAG | 18031 |
| 823 | P9R11_ins4_ | UCCGUUAUCAACUUGAAAAAGUGGCACCGAGUCGGUGCGCCUUAGUGAUCUGUGCUCG | |
| | nick_v1 | | |
| RNACS7 | B2m2_ | GAGUAGCGCGAGCACAGCUAGUUUUAGAGCUAGAAAUAGCAAGUUAAAAUAAGGCUAG | 18032 |
| 824 | P8R11_ins4_ | UCCGUUAUCAACUUGAAAAAGUGGCACCGAGUCGGUGCGCCUUAGUGAUCUGUGCUC | |
| | nick_v1 | | |
| RNACS7 | B2m2_ | GAGUAGCGCGAGCACAGCUAGUUUUAGAGCUAGAAAUAGCAAGUUAAAAUAAGGCUAG | 18033 |
| 825 | P13R13_ins4_ | UCCGUUAUCAACUUGAAAAAGUGGCACCGAGUCGGUGCUGGCCUUAGUGAUCUGUGCU | |
| | nick_v1 | CGCGCU | |
| RNACS7 | B2m2_ | GAGUAGCGCGAGCACAGCUAGUUUUAGAGCUAGAAAUAGCAAGUUAAAAUAAGGCUAG | 18034 |
| 826 | P12R13_ins4_ | UCCGUUAUCAACUUGAAAAAGUGGCACCGAGUCGGUGCUGGCCUUAGUGAUCUGUGCU | |
| | nick_v1 | CGCGC | |
| RNACS7 | B2m2_ | GAGUAGCGCGAGCACAGCUAGUUUUAGAGCUAGAAAUAGCAAGUUAAAAUAAGGCUAG | 18035 |
| 827 | P11R13_ins4_ | UCCGUUAUCAACUUGAAAAAGUGGCACCGAGUCGGUGCUGGCCUUAGUGAUCUGUGCU | |
| | nick_v1 | CGCG | |
| RNACS7 | B2m2_ | GAGUAGCGCGAGCACAGCUAGUUUUAGAGCUAGAAAUAGCAAGUUAAAAUAAGGCUAG | 18036 |
| 828 | P10R13_ins4_ | UCCGUUAUCAACUUGAAAAAGUGGCACCGAGUCGGUGCUGGCCUUAGUGAUCUGUGCU | |
| | nick_v1 | CGC | |
| RNACS7 | B2m2_ | GAGUAGCGCGAGCACAGCUAGUUUUAGAGCUAGAAAUAGCAAGUUAAAAUAAGGCUAG | 18037 |
| 829 | P9R13_ins4_ | UCCGUUAUCAACUUGAAAAAGUGGCACCGAGUCGGUGCUGGCCUUAGUGAUCUGUGCU | |
| | nick_v1 | CG | |
| RNACS7 | B2m2_ | GAGUAGCGCGAGCACAGCUAGUUUUAGAGCUAGAAAUAGCAAGUUAAAAUAAGGCUAG | 18038 |
| 830 | P8R13_ins4_ | UCCGUUAUCAACUUGAAAAAGUGGCACCGAGUCGGUGCUGGCCUUAGUGAUCUGUGCU | |
| | nick_v1 | C | |
| RNACS7 | B2m2_ | GAGUAGCGCGAGCACAGCUAGUUUUAGAGCUAGAAAUAGCAAGUUAAAAUAAGGCUAG | 18039 |
| 831 | P13R15_ins4_ | UCCGUUAUCAACUUGAAAAAGUGGCACCGAGUCGGUGCCGUGGCCUUAGUGAUCUGUG | |
| | nick_v1 | CUCGCGCU | |
| RNACS7 | B2m2_ | GAGUAGCGCGAGCACAGCUAGUUUUAGAGCUAGAAAUAGCAAGUUAAAAUAAGGCUAG | 18040 |
| 832 | P12R15_ins4_ | UCCGUUAUCAACUUGAAAAAGUGGCACCGAGUCGGUGCCGUGGCCUUAGUGAUCUGUG | |
| | nick_v1 | CUCGCGC | |
| RNACS7 | B2m2_ | GAGUAGCGCGAGCACAGCUAGUUUUAGAGCUAGAAAUAGCAAGUUAAAAUAAGGCUAG | 18041 |
| 833 | P11R15_ins4_ | UCCGUUAUCAACUUGAAAAAGUGGCACCGAGUCGGUGCCGUGGCCUUAGUGAUCUGUG | |
| | nick_v1 | CUCGCG | |
| RNACS7 | B2m2_ | GAGUAGCGCGAGCACAGCUAGUUUUAGAGCUAGAAAUAGCAAGUUAAAAUAAGGCUAG | 18042 |
| 834 | P10R15_ins4_ | UCCGUUAUCAACUUGAAAAAGUGGCACCGAGUCGGUGCCGUGGCCUUAGUGAUCUGUG | |
| | nick_v1 | CUCGC | |
| RNACS7 | B2m2_ | GAGUAGCGCGAGCACAGCUAGUUUUAGAGCUAGAAAUAGCAAGUUAAAAUAAGGCUAG | 18043 |
| 835 | P9R15_ins4_ | UCCGUUAUCAACUUGAAAAAGUGGCACCGAGUCGGUGCCGUGGCCUUAGUGAUCUGUG | |
| | nick_v1 | CUCG | |
| RNACS7 | B2m2_ | GAGUAGCGCGAGCACAGCUAGUUUUAGAGCUAGAAAUAGCAAGUUAAAAUAAGGCUAG | 18044 |
| 836 | P8R15_ins4_ | UCCGUUAUCAACUUGAAAAAGUGGCACCGAGUCGGUGCCGUGGCCUUAGUGAUCUGUG | |
| | nick_v1 | CUC | |
| RNACS7 | B2m2_ | GAGUAGCGCGAGCACAGCUAGUUUUAGAGCUAGAAAUAGCAAGUUAAAAUAAGGCUAG | 18045 |
| 837 | P13R17_ins4_ | UCCGUUAUCAACUUGAAAAAGUGGCACCGAGUCGGUGCUCCGUGGCCUUAGUGAUCUG | |
| | nick_v1 | UGCUCGCGCU | |
| RNACS7 | B2m2_ | GAGUAGCGCGAGCACAGCUAGUUUUAGAGCUAGAAAUAGCAAGUUAAAAUAAGGCUAG | 18046 |
| 838 | P12R17_ins4_ | UCCGUUAUCAACUUGAAAAAGUGGCACCGAGUCGGUGCUCCGUGGCCUUAGUGAUCUG | |
| | nick_v1 | UGCUCGCGC | |
| RNACS7 | B2m2_ | GAGUAGCGCGAGCACAGCUAGUUUUAGAGCUAGAAAUAGCAAGUUAAAAUAAGGCUAG | 18047 |
| 839 | P11R17_ins4_ | UCCGUUAUCAACUUGAAAAAGUGGCACCGAGUCGGUGCUCCGUGGCCUUAGUGAUCUG | |
| | nick_v1 | UGCUCGCG | |
| RNACS7 | B2m2_ | GAGUAGCGCGAGCACAGCUAGUUUUAGAGCUAGAAAUAGCAAGUUAAAAUAAGGCUAG | 18048 |
| 840 | P10R17_ins4_ | UCCGUUAUCAACUUGAAAAAGUGGCACCGAGUCGGUGCUCCGUGGCCUUAGUGAUCUG | |
| | nick_v1 | UGCUCGC | |
| RNACS7 | B2m2_ | GAGUAGCGCGAGCACAGCUAGUUUUAGAGCUAGAAAUAGCAAGUUAAAAUAAGGCUAG | 18049 |
| 841 | P9R17_ins4_ | UCCGUUAUCAACUUGAAAAAGUGGCACCGAGUCGGUGCUCCGUGGCCUUAGUGAUCUG | |
| | nick_v1 | UGCUCG | |
| RNACS7 | B2m2_ | GAGUAGCGCGAGCACAGCUAGUUUUAGAGCUAGAAAUAGCAAGUUAAAAUAAGGCUAG | 18050 |
| 842 | P8R17_ins4_ | UCCGUUAUCAACUUGAAAAAGUGGCACCGAGUCGGUGCUCCGUGGCCUUAGUGAUCUG | |
| | nick_v1 | UGCUC | |
| RNACS7 | B2m2_ | GAGUAGCGCGAGCACAGCUAGUUUUAGAGCUAGAAAUAGCAAGUUAAAAUAAGGCUAG | 18051 |
| 843 | P13R19_ins4_ | UCCGUUAUCAACUUGAAAAAGUGGCACCGAGUCGGUGCGCUCCGUGGCCUUAGUGAUC | |
| | nick_v1 | UGUGCUCGCGCU | |
| RNACS7 | B2m2_ | GAGUAGCGCGAGCACAGCUAGUUUUAGAGCUAGAAAUAGCAAGUUAAAAUAAGGCUAG | 18052 |
| 844 | P12R19_ins4_ | UCCGUUAUCAACUUGAAAAAGUGGCACCGAGUCGGUGCGCUCCGUGGCCUUAGUGAUC | |
| | nick_v1 | UGUGCUCGCGC | |
| RNACS7 | B2m2_ | GAGUAGCGCGAGCACAGCUAGUUUUAGAGCUAGAAAUAGCAAGUUAAAAUAAGGCUAG | 18053 |
| 845 | P11R19_ins4_ | UCCGUUAUCAACUUGAAAAAGUGGCACCGAGUCGGUGCGCUCCGUGGCCUUAGUGAUC | |
| | nick_v1 | UGUGCUCGCG | |
| RNACS7 | B2m2_ | GAGUAGCGCGAGCACAGCUAGUUUUAGAGCUAGAAAUAGCAAGUUAAAAUAAGGCUAG | 18054 |
| 846 | P10R19_ins4_ | UCCGUUAUCAACUUGAAAAAGUGGCACCGAGUCGGUGCGCUCCGUGGCCUUAGUGAUC | |
| | nick_v1 | UGUGCUCGC | |
| RNACS7 | B2m2_ | GAGUAGCGCGAGCACAGCUAGUUUUAGAGCUAGAAAUAGCAAGUUAAAAUAAGGCUAG | 18055 |
| 847 | P9R19_ins4_ | UCCGUUAUCAACUUGAAAAAGUGGCACCGAGUCGGUGCGCUCCGUGGCCUUAGUGAUC | |
| | nick_v1 | UGUGCUCG | |
| RNACS7 | B2m2_ | GAGUAGCGCGAGCACAGCUAGUUUUAGAGCUAGAAAUAGCAAGUUAAAAUAAGGCUAG | 18056 |
| 848 | P8R19_ins4_ | UCCGUUAUCAACUUGAAAAAGUGGCACCGAGUCGGUGCGCUCCGUGGCCUUAGUGAUC | |
| | nick_v1 | UGUGCUC | |
| RNACS7 | B2m2_ | GAGUAGCGCGAGCACAGCUAGUUUUAGAGCUAGAAAUAGCAAGUUAAAAUAAGGCUAG | 18057 |
| 849 | P13R21_ins4_ | UCCGUUAUCAACUUGAAAAAGUGGCACCGAGUCGGUGCUCGCUCCGUGGCCUUAGUGA | |
| | nick_v1 | UCUGUGCUCGCGCU | |
| RNACS7 | B2m2_ | GAGUAGCGCGAGCACAGCUAGUUUUAGAGCUAGAAAUAGCAAGUUAAAAUAAGGCUAG | 18058 |
| 850 | P12R21_ins4_ | UCCGUUAUCAACUUGAAAAAGUGGCACCGAGUCGGUGCUCGCUCCGUGGCCUUAGUGA | |
| | nick_v1 | UCUGUGCUCGCGC | |
| RNACS7 | B2m2_ | GAGUAGCGCGAGCACAGCUAGUUUUAGAGCUAGAAAUAGCAAGUUAAAAUAAGGCUAG | 18059 |
| 851 | P11R21_ins4_ | UCCGUUAUCAACUUGAAAAAGUGGCACCGAGUCGGUGCUCGCUCCGUGGCCUUAGUGA | |
| | nick_v1 | UCUGUGCUCGCG | |
| RNACS7 | B2m2_ | GAGUAGCGCGAGCACAGCUAGUUUUAGAGCUAGAAAUAGCAAGUUAAAAUAAGGCUAG | 18060 |
| 852 | P10R21_ins4_ | UCCGUUAUCAACUUGAAAAAGUGGCACCGAGUCGGUGCUCGCUCCGUGGCCUUAGUGA | |
| | nick_v1 | UCUGUGCUCGC | |
| RNACS7 | B2m2_ | GAGUAGCGCGAGCACAGCUAGUUUUAGAGCUAGAAAUAGCAAGUUAAAAUAAGGCUAG | 18061 |
| 853 | P9R21_ins4_ | UCCGUUAUCAACUUGAAAAAGUGGCACCGAGUCGGUGCUCGCUCCGUGGCCUUAGUGA | |
| | nick_v1 | UCUGUGCUCG | |
| RNACS7 | B2m2_ | GAGUAGCGCGAGCACAGCUAGUUUUAGAGCUAGAAAUAGCAAGUUAAAAUAAGGCUAG | 18062 |
| 854 | P8R21_ins4_ | UCCGUUAUCAACUUGAAAAAGUGGCACCGAGUCGGUGCUCGCUCCGUGGCCUUAGUGA | |
| | nick_v1 | UCUGUGCUC | |
| RNACS7 | B2m2_ | GAGUAGCGCGAGCACAGCUAGUUUUAGAGCUAGAAAUAGCAAGUUAAAAUAAGGCUAG | 18063 |
| 855 | P13R23_ins4_ | UCCGUUAUCAACUUGAAAAAGUGGCACCGAGUCGGUGCUCUCGCUCCGUGGCCUUAGU | |
| | nick_v1 | GAUCUGUGCUCGCGCU | |
| RNACS7 | B2m2_ | GAGUAGCGCGAGCACAGCUAGUUUUAGAGCUAGAAAUAGCAAGUUAAAAUAAGGCUAG | 18064 |
| 856 | P12R23_ins4_ | UCCGUUAUCAACUUGAAAAAGUGGCACCGAGUCGGUGCUCUCGCUCCGUGGCCUUAGU | |
| | nick_v1 | GAUCUGUGCUCGCGC | |
| RNACS7 | B2m2_ | GAGUAGCGCGAGCACAGCUAGUUUUAGAGCUAGAAAUAGCAAGUUAAAAUAAGGCUAG | 18065 |
| 857 | P11R23_ins4_ | UCCGUUAUCAACUUGAAAAAGUGGCACCGAGUCGGUGCUCUCGCUCCGUGGCCUUAGU | |
| | nick_v1 | GAUCUGUGCUCGCG | |
| RNACS7 | B2m2_ | GAGUAGCGCGAGCACAGCUAGUUUUAGAGCUAGAAAUAGCAAGUUAAAAUAAGGCUAG | 18066 |
| 858 | P10R23_ins4_ | UCCGUUAUCAACUUGAAAAAGUGGCACCGAGUCGGUGCUCUCGCUCCGUGGCCUUAGU | |
| | nick_v1 | GAUCUGUGCUCGC | |
| RNACS7 | B2m2_ | GAGUAGCGCGAGCACAGCUAGUUUUAGAGCUAGAAAUAGCAAGUUAAAAUAAGGCUAG | 18067 |
| 859 | P9R23_ins4_ | UCCGUUAUCAACUUGAAAAAGUGGCACCGAGUCGGUGCUCUCGCUCCGUGGCCUUAGU | |
| | nick_v1 | GAUCUGUGCUCG | |
| RNACS7 | B2m2_ | GAGUAGCGCGAGCACAGCUAGUUUUAGAGCUAGAAAUAGCAAGUUAAAAUAAGGCUAG | 18068 |
| 860 | P8R23_ins4_ | UCCGUUAUCAACUUGAAAAAGUGGCACCGAGUCGGUGCUCUCGCUCCGUGGCCUUAGU | |
| | nick_v1 | GAUCUGUGCUC | |
| RNACS7 | B2m2_ | GAGUAGCGCGAGCACAGCUAGUUUUAGAGCUAGAAAUAGCAAGUUAAAAUAAGGCUAG | 18069 |
| 861 | P13R11_ins4_ | UCCGUUAUCAACUUGAAAAAGUGGCACCGAGUCGGUGCGCCUUAGUAACCUGUGCUCG | |
| | nick_v2 | CGCU | |
| RNACS7 | B2m2_ | GAGUAGCGCGAGCACAGCUAGUUUUAGAGCUAGAAAUAGCAAGUUAAAAUAAGGCUAG | 18070 |
| 862 | P12R11_ins4_ | UCCGUUAUCAACUUGAAAAAGUGGCACCGAGUCGGUGCGCCUUAGUAACCUGUGCUCG | |
| | nick_v2 | CGC | |
| RNACS7 | B2m2_ | GAGUAGCGCGAGCACAGCUAGUUUUAGAGCUAGAAAUAGCAAGUUAAAAUAAGGCUAG | 18071 |
| 863 | P11R11_ins4_ | UCCGUUAUCAACUUGAAAAAGUGGCACCGAGUCGGUGCGCCUUAGUAACCUGUGCUCG | |
| | nick_v2 | CG | |
| RNACS7 | B2m2_ | GAGUAGCGCGAGCACAGCUAGUUUUAGAGCUAGAAAUAGCAAGUUAAAAUAAGGCUAG | 18072 |
| 864 | P10R11_ins4_ | UCCGUUAUCAACUUGAAAAAGUGGCACCGAGUCGGUGCGCCUUAGUAACCUGUGCUCG | |
| | nick_v2 | C | |
| RNACS7 | B2m2_ | GAGUAGCGCGAGCACAGCUAGUUUUAGAGCUAGAAAUAGCAAGUUAAAAUAAGGCUAG | 18073 |
| 865 | P9R11_ins4_ | UCCGUUAUCAACUUGAAAAAGUGGCACCGAGUCGGUGCGCCUUAGUAACCUGUGCUCG | |
| | nick_v2 | | |
| RNACS7 | B2m2_ | GAGUAGCGCGAGCACAGCUAGUUUUAGAGCUAGAAAUAGCAAGUUAAAAUAAGGCUAG | 18074 |
| 866 | P8R11_ins4_ | UCCGUUAUCAACUUGAAAAAGUGGCACCGAGUCGGUGCGCCUUAGUAACCUGUGCUC | |
| | nick_v2 | | |
| RNACS7 | B2m2_ | GAGUAGCGCGAGCACAGCUAGUUUUAGAGCUAGAAAUAGCAAGUUAAAAUAAGGCUAG | 18075 |
| 867 | P13R13_ins4_ | UCCGUUAUCAACUUGAAAAAGUGGCACCGAGUCGGUGCUGGCCUUAGUAACCUGUGCU | |
| | nick_v2 | CGCGCU | |
| RNACS7 | B2m2_ | GAGUAGCGCGAGCACAGCUAGUUUUAGAGCUAGAAAUAGCAAGUUAAAAUAAGGCUAG | 18076 |
| 868 | P12R13_ins4_ | UCCGUUAUCAACUUGAAAAAGUGGCACCGAGUCGGUGCUGGCCUUAGUAACCUGUGCU | |
| | nick_v2 | CGCGC | |
| RNACS7 | B2m2_ | GAGUAGCGCGAGCACAGCUAGUUUUAGAGCUAGAAAUAGCAAGUUAAAAUAAGGCUAG | 18077 |
| 869 | P11R13_ins4_ | UCCGUUAUCAACUUGAAAAAGUGGCACCGAGUCGGUGCUGGCCUUAGUAACCUGUGCU | |
| | nick_v2 | CGCG | |
| RNACS7 | B2m2_ | GAGUAGCGCGAGCACAGCUAGUUUUAGAGCUAGAAAUAGCAAGUUAAAAUAAGGCUAG | 18078 |
| 870 | P10R13_ins4_ | UCCGUUAUCAACUUGAAAAAGUGGCACCGAGUCGGUGCUGGCCUUAGUAACCUGUGCU | |
| | nick_v2 | CGC | |
| RNACS7 | B2m2_ | GAGUAGCGCGAGCACAGCUAGUUUUAGAGCUAGAAAUAGCAAGUUAAAAUAAGGCUAG | 18079 |
| 871 | P9R13_ins4_ | UCCGUUAUCAACUUGAAAAAGUGGCACCGAGUCGGUGCUGGCCUUAGUAACCUGUGCU | |
| | nick_v2 | CG | |
| RNACS7 | B2m2_ | GAGUAGCGCGAGCACAGCUAGUUUUAGAGCUAGAAAUAGCAAGUUAAAAUAAGGCUAG | 18080 |
| 872 | P8R13_ins4_ | UCCGUUAUCAACUUGAAAAAGUGGCACCGAGUCGGUGCUGGCCUUAGUAACCUGUGCU | |
| | nick_v2 | C | |
| RNACS7 | B2m2_ | GAGUAGCGCGAGCACAGCUAGUUUUAGAGCUAGAAAUAGCAAGUUAAAAUAAGGCUAG | 18081 |
| 873 | P13R15_ins4_ | UCCGUUAUCAACUUGAAAAAGUGGCACCGAGUCGGUGCCGUGGCCUUAGUAACCUGUG | |
| | nick_v2 | CUCGCGCU | |
| RNACS7 | B2m2_ | GAGUAGCGCGAGCACAGCUAGUUUUAGAGCUAGAAAUAGCAAGUUAAAAUAAGGCUAG | 18082 |
| 874 | P12R15_ins4_ | UCCGUUAUCAACUUGAAAAAGUGGCACCGAGUCGGUGCCGUGGCCUUAGUAACCUGUG | |
| | nick_v2 | CUCGCGC | |
| RNACS7 | B2m2_ | GAGUAGCGCGAGCACAGCUAGUUUUAGAGCUAGAAAUAGCAAGUUAAAAUAAGGCUAG | 18083 |
| 875 | P11R15_ins4_ | UCCGUUAUCAACUUGAAAAAGUGGCACCGAGUCGGUGCCGUGGCCUUAGUAACCUGUG | |
| | nick_v2 | CUCGCG | |
| RNACS7 | B2m2_ | GAGUAGCGCGAGCACAGCUAGUUUUAGAGCUAGAAAUAGCAAGUUAAAAUAAGGCUAG | 18084 |
| 876 | P10R15_ins4_ | UCCGUUAUCAACUUGAAAAAGUGGCACCGAGUCGGUGCCGUGGCCUUAGUAACCUGUG | |
| | nick_v2 | CUCGC | |
| RNACS7 | B2m2_ | GAGUAGCGCGAGCACAGCUAGUUUUAGAGCUAGAAAUAGCAAGUUAAAAUAAGGCUAG | 18085 |
| 877 | P9R15_ins4_ | UCCGUUAUCAACUUGAAAAAGUGGCACCGAGUCGGUGCCGUGGCCUUAGUAACCUGUG | |
| | nick_v2 | CUCG | |
| RNACS7 | B2m2_ | GAGUAGCGCGAGCACAGCUAGUUUUAGAGCUAGAAAUAGCAAGUUAAAAUAAGGCUAG | 18086 |
| 878 | P8R15_ins4_ | UCCGUUAUCAACUUGAAAAAGUGGCACCGAGUCGGUGCCGUGGCCUUAGUAACCUGUG | |
| | nick_v2 | CUC | |
| RNACS7 | B2m2_ | GAGUAGCGCGAGCACAGCUAGUUUUAGAGCUAGAAAUAGCAAGUUAAAAUAAGGCUAG | 18087 |
| 879 | P13R17_ins4_ | UCCGUUAUCAACUUGAAAAAGUGGCACCGAGUCGGUGCUCCGUGGCCUUAGUAACCUG | |
| | nick_v2 | UGCUCGCGCU | |
| RNACS7 | B2m2_ | GAGUAGCGCGAGCACAGCUAGUUUUAGAGCUAGAAAUAGCAAGUUAAAAUAAGGCUAG | 18088 |
| 880 | P12R17_ins4_ | UCCGUUAUCAACUUGAAAAAGUGGCACCGAGUCGGUGCUCCGUGGCCUUAGUAACCUG | |
| | nick_v2 | UGCUCGCGC | |
| RNACS7 | B2m2_ | GAGUAGCGCGAGCACAGCUAGUUUUAGAGCUAGAAAUAGCAAGUUAAAAUAAGGCUAG | 18089 |
| 881 | P11R17_ins4_ | UCCGUUAUCAACUUGAAAAAGUGGCACCGAGUCGGUGCUCCGUGGCCUUAGUAACCUG | |
| | nick_v2 | UGCUCGCG | |
| RNACS7 | B2m2_ | GAGUAGCGCGAGCACAGCUAGUUUUAGAGCUAGAAAUAGCAAGUUAAAAUAAGGCUAG | 18090 |
| 882 | P10R17_ins4_ | UCCGUUAUCAACUUGAAAAAGUGGCACCGAGUCGGUGCUCCGUGGCCUUAGUAACCUG | |
| | nick_v2 | UGCUCGC | |
| RNACS7 | B2m2_ | GAGUAGCGCGAGCACAGCUAGUUUUAGAGCUAGAAAUAGCAAGUUAAAAUAAGGCUAG | 18091 |
| 883 | P9R17_ins4_ | UCCGUUAUCAACUUGAAAAAGUGGCACCGAGUCGGUGCUCCGUGGCCUUAGUAACCUG | |
| | nick_v2 | UGCUCG | |
| RNACS7 | B2m2_ | GAGUAGCGCGAGCACAGCUAGUUUUAGAGCUAGAAAUAGCAAGUUAAAAUAAGGCUAG | 18092 |
| 884 | P8R17_ins4_ | UCCGUUAUCAACUUGAAAAAGUGGCACCGAGUCGGUGCUCCGUGGCCUUAGUAACCUG | |
| | nick_v2 | UGCUC | |
| RNACS7 | B2m2_ | GAGUAGCGCGAGCACAGCUAGUUUUAGAGCUAGAAAUAGCAAGUUAAAAUAAGGCUAG | 18093 |
| 885 | P13R19_ins4_ | UCCGUUAUCAACUUGAAAAAGUGGCACCGAGUCGGUGCGCUCCGUGGCCUUAGUAACC | |
| | nick_v2 | UGUGCUCGCGCU | |
| RNACS7 | B2m2_ | GAGUAGCGCGAGCACAGCUAGUUUUAGAGCUAGAAAUAGCAAGUUAAAAUAAGGCUAG | 18094 |
| 886 | P12R19_ins4_ | UCCGUUAUCAACUUGAAAAAGUGGCACCGAGUCGGUGCGCUCCGUGGCCUUAGUAACC | |
| | nick_v2 | UGUGCUCGCGC | |
| RNACS7 | B2m2_ | GAGUAGCGCGAGCACAGCUAGUUUUAGAGCUAGAAAUAGCAAGUUAAAAUAAGGCUAG | 18095 |
| 887 | P11R19_ins4_ | UCCGUUAUCAACUUGAAAAAGUGGCACCGAGUCGGUGCGCUCCGUGGCCUUAGUAACC | |
| | nick_v2 | UGUGCUCGCG | |
| RNACS7 | B2m2_P10 | GAGUAGCGCGAGCACAGCUAGUUUUAGAGCUAGAAAUAGCAAGUUAAAAUAAGGCUAG | 18096 |
| 888 | R19_ins4_ni | UCCGUUAUCAACUUGAAAAAGUGGCACCGAGUCGGUGCGCUCCGUGGCCUUAGUAACC | |
| | ck_v2 | UGUGCUCGC | |
| RNACS7 | B2m2_ | GAGUAGCGCGAGCACAGCUAGUUUUAGAGCUAGAAAUAGCAAGUUAAAAUAAGGCUAG | 18097 |
| 889 | P9R19_ins4_ | UCCGUUAUCAACUUGAAAAAGUGGCACCGAGUCGGUGCGCUCCGUGGCCUUAGUAACC | |
| | nick_v2 | UGUGCUCG | |
| RNACS7 | B2m2_ | GAGUAGCGCGAGCACAGCUAGUUUUAGAGCUAGAAAUAGCAAGUUAAAAUAAGGCUAG | 18098 |
| 890 | P8R19_ins4_ | UCCGUUAUCAACUUGAAAAAGUGGCACCGAGUCGGUGCGCUCCGUGGCCUUAGUAACC | |
| | nick_v2 | UGUGCUC | |
| RNACS7 | B2m2_ | GAGUAGCGCGAGCACAGCUAGUUUUAGAGCUAGAAAUAGCAAGUUAAAAUAAGGCUAG | 18099 |
| 891 | P13R21_ins4_ | UCCGUUAUCAACUUGAAAAAGUGGCACCGAGUCGGUGCUCGCUCCGUGGCCUUAGUAA | |
| | nick_v2 | CCUGUGCUCGCGCU | |
| RNACS7 | B2m2_ | GAGUAGCGCGAGCACAGCUAGUUUUAGAGCUAGAAAUAGCAAGUUAAAAUAAGGCUAG | 18100 |
| 892 | P12R21_ins4_ | UCCGUUAUCAACUUGAAAAAGUGGCACCGAGUCGGUGCUCGCUCCGUGGCCUUAGUAA | |
| | nick_v2 | CCUGUGCUCGCGC | |
| RNACS7 | B2m2_ | GAGUAGCGCGAGCACAGCUAGUUUUAGAGCUAGAAAUAGCAAGUUAAAAUAAGGCUAG | 18101 |
| 893 | P11R21_ins4_ | UCCGUUAUCAACUUGAAAAAGUGGCACCGAGUCGGUGCUCGCUCCGUGGCCUUAGUAA | |
| | nick_v2 | CCUGUGCUCGCG | |
| RNACS7 | B2m2_ | GAGUAGCGCGAGCACAGCUAGUUUUAGAGCUAGAAAUAGCAAGUUAAAAUAAGGCUAG | 18102 |
| 894 | P10R21_ins4_ | UCCGUUAUCAACUUGAAAAAGUGGCACCGAGUCGGUGCUCGCUCCGUGGCCUUAGUAA | |
| | nick_v2 | CCUGUGCUCGC | |
| RNACS7 | B2m2_ | GAGUAGCGCGAGCACAGCUAGUUUUAGAGCUAGAAAUAGCAAGUUAAAAUAAGGCUAG | 18103 |
| 895 | P9R21_ins4_ | UCCGUUAUCAACUUGAAAAAGUGGCACCGAGUCGGUGCUCGCUCCGUGGCCUUAGUAA | |
| | nick_v2 | CCUGUGCUCG | |
| RNACS7 | B2m2_ | GAGUAGCGCGAGCACAGCUAGUUUUAGAGCUAGAAAUAGCAAGUUAAAAUAAGGCUAG | 18104 |
| 896 | P8R21_ins4_ | UCCGUUAUCAACUUGAAAAAGUGGCACCGAGUCGGUGCUCGCUCCGUGGCCUUAGUAA | |
| | nick_v2 | CCUGUGCUC | |
| RNACS7 | B2m2_ | GAGUAGCGCGAGCACAGCUAGUUUUAGAGCUAGAAAUAGCAAGUUAAAAUAAGGCUAG | 18105 |
| 897 | P13R23_ins4_ | UCCGUUAUCAACUUGAAAAAGUGGCACCGAGUCGGUGCUCUCGCUCCGUGGCCUUAGU | |
| | nick_v2 | AACCUGUGCUCGCGCU | |
| RNACS7 | B2m2_ | GAGUAGCGCGAGCACAGCUAGUUUUAGAGCUAGAAAUAGCAAGUUAAAAUAAGGCUAG | 18106 |
| 898 | P12R23_ins4_ | UCCGUUAUCAACUUGAAAAAGUGGCACCGAGUCGGUGCUCUCGCUCCGUGGCCUUAGU | |
| | nick_v2 | AACCUGUGCUCGCGC | |
| RNACS7 | B2m2_ | GAGUAGCGCGAGCACAGCUAGUUUUAGAGCUAGAAAUAGCAAGUUAAAAUAAGGCUAG | 18107 |
| 899 | P11R23_ins4_ | UCCGUUAUCAACUUGAAAAAGUGGCACCGAGUCGGUGCUCUCGCUCCGUGGCCUUAGU | |
| | nick_v2 | AACCUGUGCUCGCG | |
| RNACS7 | B2m2_ | GAGUAGCGCGAGCACAGCUAGUUUUAGAGCUAGAAAUAGCAAGUUAAAAUAAGGCUAG | 18108 |
| 900 | P10R23_ins4_ | UCCGUUAUCAACUUGAAAAAGUGGCACCGAGUCGGUGCUCUCGCUCCGUGGCCUUAGU | |
| | nick_v2 | AACCUGUGCUCGC | |
| RNACS7 | B2m2_ | GAGUAGCGCGAGCACAGCUAGUUUUAGAGCUAGAAAUAGCAAGUUAAAAUAAGGCUAG | 18109 |
| 901 | P9R23_ins4_ | UCCGUUAUCAACUUGAAAAAGUGGCACCGAGUCGGUGCUCUCGCUCCGUGGCCUUAGU | |
| | nick_v2 | AACCUGUGCUCG | |
| RNACS7 | B2m2_ | GAGUAGCGCGAGCACAGCUAGUUUUAGAGCUAGAAAUAGCAAGUUAAAAUAAGGCUAG | 18110 |
| 902 | P8R23_ins4_ | UCCGUUAUCAACUUGAAAAAGUGGCACCGAGUCGGUGCUCUCGCUCCGUGGCCUUAGU | |
| | knic_v2 | AACCUGUGCUC | |
| RNACS7 | B2m2_ | GAGUAGCGCGAGCACAGCUAGUUUUAGAGCUAGAAAUAGCAAGUUAAAAUAAGGCUAG | 18111 |
| 903 | P13R12_ins5_ | UCCGUUAUCAACUUGAAAAAGUGGCACCGAGUCGGUGCGCCUUAGUGAUGCUGUGCUC | |
| | nick_v1 | GCGCU | |
| RNACS7 | B2m2_ | GAGUAGCGCGAGCACAGCUAGUUUUAGAGCUAGAAAUAGCAAGUUAAAAUAAGGCUAG | 18112 |
| 904 | P12R12_ins5_ | UCCGUUAUCAACUUGAAAAAGUGGCACCGAGUCGGUGCGCCUUAGUGAUGCUGUGCUC | |
| | nick_v1 | GCGC | |
| RNACS7 | B2m2_ | GAGUAGCGCGAGCACAGCUAGUUUUAGAGCUAGAAAUAGCAAGUUAAAAUAAGGCUAG | 18113 |
| 905 | P11R12_ins5_ | UCCGUUAUCAACUUGAAAAAGUGGCACCGAGUCGGUGCGCCUUAGUGAUGCUGUGCUC | |
| | nick_v1 | GCG | |
| RNACS7 | B2m2_ | GAGUAGCGCGAGCACAGCUAGUUUUAGAGCUAGAAAUAGCAAGUUAAAAUAAGGCUAG | 18114 |
| 906 | P10R12_ins5_ | UCCGUUAUCAACUUGAAAAAGUGGCACCGAGUCGGUGCGCCUUAGUGAUGCUGUGCUC | |
| | nick_v1 | GC | |
| RNACS7 | B2m2_ | GAGUAGCGCGAGCACAGCUAGUUUUAGAGCUAGAAAUAGCAAGUUAAAAUAAGGCUAG | 18115 |
| 907 | P9R12_ins5_ | UCCGUUAUCAACUUGAAAAAGUGGCACCGAGUCGGUGCGCCUUAGUGAUGCUGUGCUC | |
| | nick_v1 | G | |
| RNACS7 | B2m2_ | GAGUAGCGCGAGCACAGCUAGUUUUAGAGCUAGAAAUAGCAAGUUAAAAUAAGGCUAG | 18116 |
| 908 | P8R12_ins5_ | UCCGUUAUCAACUUGAAAAAGUGGCACCGAGUCGGUGCGCCUUAGUGAUGCUGUGCUC | |
| | nick_v1 | | |
| RNACS7 | B2m2_ | GAGUAGCGCGAGCACAGCUAGUUUUAGAGCUAGAAAUAGCAAGUUAAAAUAAGGCUAG | 18117 |
| 909 | P13R14_ins5_ | UCCGUUAUCAACUUGAAAAAGUGGCACCGAGUCGGUGCUGGCCUUAGUGAUGCUGUGC | |
| | nick_v1 | UCGCGCU | |
| RNACS7 | B2m2_ | GAGUAGCGCGAGCACAGCUAGUUUUAGAGCUAGAAAUAGCAAGUUAAAAUAAGGCUAG | 18118 |
| 910 | P12R14_ins5_ | UCCGUUAUCAACUUGAAAAAGUGGCACCGAGUCGGUGCUGGCCUUAGUGAUGCUGUGC | |
| | nick_v1 | UCGCGC | |
| RNACS7 | B2m2_ | GAGUAGCGCGAGCACAGCUAGUUUUAGAGCUAGAAAUAGCAAGUUAAAAUAAGGCUAG | 18119 |
| 911 | P11R14_ins5_ | UCCGUUAUCAACUUGAAAAAGUGGCACCGAGUCGGUGCUGGCCUUAGUGAUGCUGUGC | |
| | nick_v1 | UCGCG | |
| RNACS7 | B2m2_ | GAGUAGCGCGAGCACAGCUAGUUUUAGAGCUAGAAAUAGCAAGUUAAAAUAAGGCUAG | 18120 |
| 912 | P10R14_ins5_ | UCCGUUAUCAACUUGAAAAAGUGGCACCGAGUCGGUGCUGGCCUUAGUGAUGCUGUGC | |
| | nick_v1 | UCGC | |
| RNACS7 | B2m2_ | GAGUAGCGCGAGCACAGCUAGUUUUAGAGCUAGAAAUAGCAAGUUAAAAUAAGGCUAG | 18121 |
| 913 | P9R14_ins5_ | UCCGUUAUCAACUUGAAAAAGUGGCACCGAGUCGGUGCUGGCCUUAGUGAUGCUGUGC | |
| | nick_v1 | UCG | |
| RNACS7 | B2m2_ | GAGUAGCGCGAGCACAGCUAGUUUUAGAGCUAGAAAUAGCAAGUUAAAAUAAGGCUAG | 18122 |
| 914 | P8R14_ins5_ | UCCGUUAUCAACUUGAAAAAGUGGCACCGAGUCGGUGCUGGCCUUAGUGAUGCUGUGC | |
| | nick_v1 | UC | |
| RNACS7 | B2m2_ | GAGUAGCGCGAGCACAGCUAGUUUUAGAGCUAGAAAUAGCAAGUUAAAAUAAGGCUAG | 18123 |
| 915 | RP1316_ins5_ | UCCGUUAUCAACUUGAAAAAGUGGCACCGAGUCGGUGCCGUGGCCUUAGUGAUGCUGU | |
| | nick_v1 | GCUCGCGCU | |
| RNACS7 | B2m2_ | GAGUAGCGCGAGCACAGCUAGUUUUAGAGCUAGAAAUAGCAAGUUAAAAUAAGGCUAG | 18124 |
| 916 | P12R16_ins5_ | UCCGUUAUCAACUUGAAAAAGUGGCACCGAGUCGGUGCCGUGGCCUUAGUGAUGCUGU | |
| | nick_v1 | GCUCGCGC | |
| RNACS7 | B2m2_ | GAGUAGCGCGAGCACAGCUAGUUUUAGAGCUAGAAAUAGCAAGUUAAAAUAAGGCUAG | 18125 |
| 917 | P11R16_ins5_ | UCCGUUAUCAACUUGAAAAAGUGGCACCGAGUCGGUGCCGUGGCCUUAGUGAUGCUGU | |
| | nick_v1 | GCUCGCG | |
| RNACS7 | B2m2_ | GAGUAGCGCGAGCACAGCUAGUUUUAGAGCUAGAAAUAGCAAGUUAAAAUAAGGCUAG | 18126 |
| 918 | P10R16_ins5_ | UCCGUUAUCAACUUGAAAAAGUGGCACCGAGUCGGUGCCGUGGCCUUAGUGAUGCUGU | |
| | nick_v1 | GCUCGC | |
| RNACS7 | B2m2_ | GAGUAGCGCGAGCACAGCUAGUUUUAGAGCUAGAAAUAGCAAGUUAAAAUAAGGCUAG | 18127 |
| 919 | P9R16_ins5_ | UCCGUUAUCAACUUGAAAAAGUGGCACCGAGUCGGUGCCGUGGCCUUAGUGAUGCUGU | |
| | nick_v1 | GCUCG | |
| RNACS7 | B2m2_ | GAGUAGCGCGAGCACAGCUAGUUUUAGAGCUAGAAAUAGCAAGUUAAAAUAAGGCUAG | 18128 |
| 920 | P8R16_ins5_ | UCCGUUAUCAACUUGAAAAAGUGGCACCGAGUCGGUGCCGUGGCCUUAGUGAUGCUGU | |
| | nick_v1 | GCUC | |
| RNACS7 | B2m2_ | GAGUAGCGCGAGCACAGCUAGUUUUAGAGCUAGAAAUAGCAAGUUAAAAUAAGGCUAG | 18129 |
| 921 | P13R18_ins5_ | UCCGUUAUCAACUUGAAAAAGUGGCACCGAGUCGGUGCUCCGUGGCCUUAGUGAUGCU | |
| | nick_v1 | GUGCUCGCGCU | |
| RNACS7 | B2m2_ | GAGUAGCGCGAGCACAGCUAGUUUUAGAGCUAGAAAUAGCAAGUUAAAAUAAGGCUAG | 18130 |
| 922 | P12R18_ins5_ | UCCGUUAUCAACUUGAAAAAGUGGCACCGAGUCGGUGCUCCGUGGCCUUAGUGAUGCU | |
| | nick_v1 | GUGCUCGCGC | |
| RNACS7 | B2m2_ | GAGUAGCGCGAGCACAGCUAGUUUUAGAGCUAGAAAUAGCAAGUUAAAAUAAGGCUAG | 18131 |
| 923 | P11R18_ins5_ | UCCGUUAUCAACUUGAAAAAGUGGCACCGAGUCGGUGCUCCGUGGCCUUAGUGAUGCU | |
| | nick_v1 | GUGCUCGCG | |
| RNACS7 | B2m2_ | GAGUAGCGCGAGCACAGCUAGUUUUAGAGCUAGAAAUAGCAAGUUAAAAUAAGGCUAG | 18132 |
| 924 | P10R18_ins5_ | UCCGUUAUCAACUUGAAAAAGUGGCACCGAGUCGGUGCUCCGUGGCCUUAGUGAUGCU | |
| | nick_v1 | GUGCUCGC | |
| RNACS7 | B2m2_ | GAGUAGCGCGAGCACAGCUAGUUUUAGAGCUAGAAAUAGCAAGUUAAAAUAAGGCUAG | 18133 |
| 925 | P9R18_ins5_ | UCCGUUAUCAACUUGAAAAAGUGGCACCGAGUCGGUGCUCCGUGGCCUUAGUGAUGCU | |
| | nick_v1 | GUGCUCG | |
| RNACS7 | B2m2_ | GAGUAGCGCGAGCACAGCUAGUUUUAGAGCUAGAAAUAGCAAGUUAAAAUAAGGCUAG | 18134 |
| 926 | P8R18_ins5_ | UCCGUUAUCAACUUGAAAAAGUGGCACCGAGUCGGUGCUCCGUGGCCUUAGUGAUGCU | |
| | nick_v1 | GUGCUC | |
| RNACS7 | B2m2_ | GAGUAGCGCGAGCACAGCUAGUUUUAGAGCUAGAAAUAGCAAGUUAAAAUAAGGCUAG | 18135 |
| 927 | P13R20_ins5_ | UCCGUUAUCAACUUGAAAAAGUGGCACCGAGUCGGUGCGCUCCGUGGCCUUAGUGAUG | |
| | nick_v1 | CUGUGCUCGCGCU | |
| RNACS7 | B2m2_ | GAGUAGCGCGAGCACAGCUAGUUUUAGAGCUAGAAAUAGCAAGUUAAAAUAAGGCUAG | 18136 |
| 928 | P12R20_ins5_ | UCCGUUAUCAACUUGAAAAAGUGGCACCGAGUCGGUGCGCUCCGUGGCCUUAGUGAUG | |
| | nick_v1 | CUGUGCUCGCGC | |
| RNACS7 | B2m2_ | GAGUAGCGCGAGCACAGCUAGUUUUAGAGCUAGAAAUAGCAAGUUAAAAUAAGGCUAG | 18137 |
| 929 | P11R20_ins5_ | UCCGUUAUCAACUUGAAAAAGUGGCACCGAGUCGGUGCGCUCCGUGGCCUUAGUGAUG | |
| | nick_v1 | CUGUGCUCGCG | |
| RNACS7 | B2m2_ | GAGUAGCGCGAGCACAGCUAGUUUUAGAGCUAGAAAUAGCAAGUUAAAAUAAGGCUAG | 18138 |
| 930 | P10R20_ins5_ | UCCGUUAUCAACUUGAAAAAGUGGCACCGAGUCGGUGCGCUCCGUGGCCUUAGUGAUG | |
| | nick_v1 | CUGUGCUCGC | |
| RNACS7 | B2m2_ | GAGUAGCGCGAGCACAGCUAGUUUUAGAGCUAGAAAUAGCAAGUUAAAAUAAGGCUAG | 18139 |
| 931 | P9R20_ins5_ | UCCGUUAUCAACUUGAAAAAGUGGCACCGAGUCGGUGCGCUCCGUGGCCUUAGUGAUG | |
| | nick_v1 | CUGUGCUCG | |
| RNACS7 | B2m2_ | GAGUAGCGCGAGCACAGCUAGUUUUAGAGCUAGAAAUAGCAAGUUAAAAUAAGGCUAG | 18140 |
| 932 | P8R20_ins5_ | UCCGUUAUCAACUUGAAAAAGUGGCACCGAGUCGGUGCGCUCCGUGGCCUUAGUGAUG | |
| | nick_v1 | CUGUGCUC | |
| RNACS7 | B2m2_ | GAGUAGCGCGAGCACAGCUAGUUUUAGAGCUAGAAAUAGCAAGUUAAAAUAAGGCUAG | 18141 |
| 933 | P13R22_ins5_ | UCCGUUAUCAACUUGAAAAAGUGGCACCGAGUCGGUGCUCGCUCCGUGGCCUUAGUGA | |
| | nick_v1 | UGCUGUGCUCGCGCU | |
| RNACS7 | B2m2_ | GAGUAGCGCGAGCACAGCUAGUUUUAGAGCUAGAAAUAGCAAGUUAAAAUAAGGCUAG | 18142 |
| 934 | P12R22_ins5_ | UCCGUUAUCAACUUGAAAAAGUGGCACCGAGUCGGUGCUCGCUCCGUGGCCUUAGUGA | |
| | nick_v1 | UGCUGUGCUCGCGC | |
| RNACS7 | B2m2_ | GAGUAGCGCGAGCACAGCUAGUUUUAGAGCUAGAAAUAGCAAGUUAAAAUAAGGCUAG | 18143 |
| 935 | P11R22_ins5_ | UCCGUUAUCAACUUGAAAAAGUGGCACCGAGUCGGUGCUCGCUCCGUGGCCUUAGUGA | |
| | nick_v1 | UGCUGUGCUCGCG | |
| RNACS7 | B2m2_ | GAGUAGCGCGAGCACAGCUAGUUUUAGAGCUAGAAAUAGCAAGUUAAAAUAAGGCUAG | 18144 |
| 936 | P10R22_ins5_ | UCCGUUAUCAACUUGAAAAAGUGGCACCGAGUCGGUGCUCGCUCCGUGGCCUUAGUGA | |
| | nick_v1 | UGCUGUGCUCGC | |
| RNACS7 | B2m2_ | GAGUAGCGCGAGCACAGCUAGUUUUAGAGCUAGAAAUAGCAAGUUAAAAUAAGGCUAG | 18145 |
| 937 | P9R22_ins5_ | UCCGUUAUCAACUUGAAAAAGUGGCACCGAGUCGGUGCUCGCUCCGUGGCCUUAGUGA | |
| | nick_v1 | UGCUGUGCUCG | |
| RNACS7 | B2m2_ | GAGUAGCGCGAGCACAGCUAGUUUUAGAGCUAGAAAUAGCAAGUUAAAAUAAGGCUAG | 18146 |
| 938 | P8R22_ins5_ | UCCGUUAUCAACUUGAAAAAGUGGCACCGAGUCGGUGCUCGCUCCGUGGCCUUAGUGA | |
| | nick_v1 | UGCUGUGCUC | |
| RNACS7 | B2m2_ | GAGUAGCGCGAGCACAGCUAGUUUUAGAGCUAGAAAUAGCAAGUUAAAAUAAGGCUAG | 18147 |
| 939 | P13R24_ins5_ | UCCGUUAUCAACUUGAAAAAGUGGCACCGAGUCGGUGCUCUCGCUCCGUGGCCUUAGU | |
| | nick_v1 | GAUGCUGUGCUCGCGCU | |
| RNACS7 | B2m2_ | GAGUAGCGCGAGCACAGCUAGUUUUAGAGCUAGAAAUAGCAAGUUAAAAUAAGGCUAG | 18148 |
| 940 | P12R24_ins5_ | UCCGUUAUCAACUUGAAAAAGUGGCACCGAGUCGGUGCUCUCGCUCCGUGGCCUUAGU | |
| | nick_v1 | GAUGCUGUGCUCGCGC | |
| RNACS7 | B2m2_ | GAGUAGCGCGAGCACAGCUAGUUUUAGAGCUAGAAAUAGCAAGUUAAAAUAAGGCUAG | 18149 |
| 941 | P11R24_ins5_ | UCCGUUAUCAACUUGAAAAAGUGGCACCGAGUCGGUGCUCUCGCUCCGUGGCCUUAGU | |
| | nick_v1 | GAUGCUGUGCUCGCG | |
| RNACS7 | B2m2_ | GAGUAGCGCGAGCACAGCUAGUUUUAGAGCUAGAAAUAGCAAGUUAAAAUAAGGCUAG | 18150 |
| 942 | P10R24_ins5_ | UCCGUUAUCAACUUGAAAAAGUGGCACCGAGUCGGUGCUCUCGCUCCGUGGCCUUAGU | |
| | nick_v1 | GAUGCUGUGCUCGC | |
| RNACS7 | B2m2_ | GAGUAGCGCGAGCACAGCUAGUUUUAGAGCUAGAAAUAGCAAGUUAAAAUAAGGCUAG | 18151 |
| 943 | P9R24_ins5_ | UCCGUUAUCAACUUGAAAAAGUGGCACCGAGUCGGUGCUCUCGCUCCGUGGCCUUAGU | |
| | nick_v1 | GAUGCUGUGCUCG | |
| RNACS7 | B2m2_ | GAGUAGCGCGAGCACAGCUAGUUUUAGAGCUAGAAAUAGCAAGUUAAAAUAAGGCUAG | 18152 |
| 944 | P8R24_ins5_ | UCCGUUAUCAACUUGAAAAAGUGGCACCGAGUCGGUGCUCUCGCUCCGUGGCCUUAGU | |
| | nick_v1 | GAUGCUGUGCUC | |
| RNACS7 | B2m2_ | GAGUAGCGCGAGCACAGCUAGUUUUAGAGCUAGAAAUAGCAAGUUAAAAUAAGGCUAG | 18153 |
| 945 | P13R12_ins5_ | UCCGUUAUCAACUUGAAAAAGUGGCACCGAGUCGGUGCGCCUUAGUAACCCUGUGCUC | |
| | nick_v2 | GCGCU | |
| RNACS7 | B2m2_ | GAGUAGCGCGAGCACAGCUAGUUUUAGAGCUAGAAAUAGCAAGUUAAAAUAAGGCUAG | 18154 |
| 946 | P12R12_ins5_ | UCCGUUAUCAACUUGAAAAAGUGGCACCGAGUCGGUGCGCCUUAGUAACCCUGUGCUC | |
| | nick_v2 | GCGC | |
| RNACS7 | B2m2_ | GAGUAGCGCGAGCACAGCUAGUUUUAGAGCUAGAAAUAGCAAGUUAAAAUAAGGCUAG | 18155 |
| 947 | P11R12_ins5_ | UCCGUUAUCAACUUGAAAAAGUGGCACCGAGUCGGUGCGCCUUAGUAACCCUGUGCUC | |
| | nick_v2 | GCG | |
| RNACS7 | B2m2_ | GAGUAGCGCGAGCACAGCUAGUUUUAGAGCUAGAAAUAGCAAGUUAAAAUAAGGCUAG | 18156 |
| 948 | P10R12_ins5_ | UCCGUUAUCAACUUGAAAAAGUGGCACCGAGUCGGUGCGCCUUAGUAACCCUGUGCUC | |
| | nick_v2 | GC | |
| RNACS7 | B2m2_ | GAGUAGCGCGAGCACAGCUAGUUUUAGAGCUAGAAAUAGCAAGUUAAAAUAAGGCUAG | 18157 |
| 949 | P9R12_ins5_ | UCCGUUAUCAACUUGAAAAAGUGGCACCGAGUCGGUGCGCCUUAGUAACCCUGUGCUC | |
| | nick_v2 | G | |
| RNACS7 | B2m2_ | GAGUAGCGCGAGCACAGCUAGUUUUAGAGCUAGAAAUAGCAAGUUAAAAUAAGGCUAG | 18158 |
| 950 | P8R12_ins5_ | UCCGUUAUCAACUUGAAAAAGUGGCACCGAGUCGGUGCGCCUUAGUAACCCUGUGCUC | |
| | nick_v2 | | |
| RNACS7 | B2m2_ | GAGUAGCGCGAGCACAGCUAGUUUUAGAGCUAGAAAUAGCAAGUUAAAAUAAGGCUAG | 18159 |
| 951 | P13R14_ins5_ | UCCGUUAUCAACUUGAAAAAGUGGCACCGAGUCGGUGCUGGCCUUAGUAACCCUGUGC | |
| | nick_v2 | UCGCGCU | |
| RNACS7 | B2m2_ | GAGUAGCGCGAGCACAGCUAGUUUUAGAGCUAGAAAUAGCAAGUUAAAAUAAGGCUAG | 18160 |
| 952 | P12R14_ins5_ | UCCGUUAUCAACUUGAAAAAGUGGCACCGAGUCGGUGCUGGCCUUAGUAACCCUGUGC | |
| | nick_v2 | UCGCGC | |
| RNACS7 | B2m2_ | GAGUAGCGCGAGCACAGCUAGUUUUAGAGCUAGAAAUAGCAAGUUAAAAUAAGGCUAG | 18161 |
| 953 | P11R14_ins5_ | UCCGUUAUCAACUUGAAAAAGUGGCACCGAGUCGGUGCUGGCCUUAGUAACCCUGUGC | |
| | nick_v2 | UCGCG | |
| RNACS7 | B2m2_ | GAGUAGCGCGAGCACAGCUAGUUUUAGAGCUAGAAAUAGCAAGUUAAAAUAAGGCUAG | 18162 |
| 954 | P10R14_ins5_ | UCCGUUAUCAACUUGAAAAAGUGGCACCGAGUCGGUGCUGGCCUUAGUAACCCUGUGC | |
| | nick_v2 | UCGC | |
| RNACS7 | B2m2_ | GAGUAGCGCGAGCACAGCUAGUUUUAGAGCUAGAAAUAGCAAGUUAAAAUAAGGCUAG | 18163 |
| 955 | P9R14_ins5_ | UCCGUUAUCAACUUGAAAAAGUGGCACCGAGUCGGUGCUGGCCUUAGUAACCCUGUGC | |
| | nick_v2 | UCG | |
| RNACS7 | B2m2_ | GAGUAGCGCGAGCACAGCUAGUUUUAGAGCUAGAAAUAGCAAGUUAAAAUAAGGCUAG | 18164 |
| 956 | P8R14_ins5_ | UCCGUUAUCAACUUGAAAAAGUGGCACCGAGUCGGUGCUGGCCUUAGUAACCCUGUGC | |
| | nick_v2 | UC | |
| RNACS7 | B2m2_ | GAGUAGCGCGAGCACAGCUAGUUUUAGAGCUAGAAAUAGCAAGUUAAAAUAAGGCUAG | 18165 |
| 957 | P13R16_ins5_ | UCCGUUAUCAACUUGAAAAAGUGGCACCGAGUCGGUGCCGUGGCCUUAGUAACCCUGU | |
| | nick_v2 | GCUCGCGCU | |
| RNACS7 | B2m2_ | GAGUAGCGCGAGCACAGCUAGUUUUAGAGCUAGAAAUAGCAAGUUAAAAUAAGGCUAG | 18166 |
| 958 | P12R16_ins5_ | UCCGUUAUCAACUUGAAAAAGUGGCACCGAGUCGGUGCCGUGGCCUUAGUAACCCUGU | |
| | nick_v2 | GCUCGCGC | |
| RNACS7 | B2m2_ | GAGUAGCGCGAGCACAGCUAGUUUUAGAGCUAGAAAUAGCAAGUUAAAAUAAGGCUAG | 18167 |
| 959 | P11R16_ins5_ | UCCGUUAUCAACUUGAAAAAGUGGCACCGAGUCGGUGCCGUGGCCUUAGUAACCCUGU | |
| | nick_v2 | GCUCGCG | |
| RNACS7 | B2m2_ | GAGUAGCGCGAGCACAGCUAGUUUUAGAGCUAGAAAUAGCAAGUUAAAAUAAGGCUAG | 18168 |
| 960 | P10R16_ins5_ | UCCGUUAUCAACUUGAAAAAGUGGCACCGAGUCGGUGCCGUGGCCUUAGUAACCCUGU | |
| | nick_v2 | GCUCGC | |
| RNACS7 | B2m2_ | GAGUAGCGCGAGCACAGCUAGUUUUAGAGCUAGAAAUAGCAAGUUAAAAUAAGGCUAG | 18169 |
| 961 | P9R16_ins5_ | UCCGUUAUCAACUUGAAAAAGUGGCACCGAGUCGGUGCCGUGGCCUUAGUAACCCUGU | |
| | nick_v2 | GCUCG | |
| RNACS7 | B2m2_ | GAGUAGCGCGAGCACAGCUAGUUUUAGAGCUAGAAAUAGCAAGUUAAAAUAAGGCUAG | 18170 |
| 962 | P8R16_ins5_ | UCCGUUAUCAACUUGAAAAAGUGGCACCGAGUCGGUGCCGUGGCCUUAGUAACCCUGU | |
| | nick_v2 | GCUC | |
| RNACS7 | B2m2_ | GAGUAGCGCGAGCACAGCUAGUUUUAGAGCUAGAAAUAGCAAGUUAAAAUAAGGCUAG | 18171 |
| 963 | P13R18_ins5_ | UCCGUUAUCAACUUGAAAAAGUGGCACCGAGUCGGUGCUCCGUGGCCUUAGUAACCCU | |
| | nick_v2 | GUGCUCGCGCU | |
| RNACS7 | B2m2_ | GAGUAGCGCGAGCACAGCUAGUUUUAGAGCUAGAAAUAGCAAGUUAAAAUAAGGCUAG | 18172 |
| 964 | P12R18_ins5_ | UCCGUUAUCAACUUGAAAAAGUGGCACCGAGUCGGUGCUCCGUGGCCUUAGUAACCCU | |
| | nick_v2 | GUGCUCGCGC | |
| RNACS7 | B2m2_ | GAGUAGCGCGAGCACAGCUAGUUUUAGAGCUAGAAAUAGCAAGUUAAAAUAAGGCUAG | 18173 |
| 965 | P11R18_ins5_ | UCCGUUAUCAACUUGAAAAAGUGGCACCGAGUCGGUGCUCCGUGGCCUUAGUAACCCU | |
| | nick_v2 | GUGCUCGCG | |
| RNACS7 | B2m2_ | GAGUAGCGCGAGCACAGCUAGUUUUAGAGCUAGAAAUAGCAAGUUAAAAUAAGGCUAG | 18174 |
| 966 | P10R18_ins5_ | UCCGUUAUCAACUUGAAAAAGUGGCACCGAGUCGGUGCUCCGUGGCCUUAGUAACCCU | |
| | nick_v2 | GUGCUCGC | |
| RNACS7 | B2m2_ | GAGUAGCGCGAGCACAGCUAGUUUUAGAGCUAGAAAUAGCAAGUUAAAAUAAGGCUAG | 18175 |
| 967 | P9R18_ins5_ | UCCGUUAUCAACUUGAAAAAGUGGCACCGAGUCGGUGCUCCGUGGCCUUAGUAACCCU | |
| | nick_v2 | GUGCUCG | |
| RNACS7 | B2m2_ | GAGUAGCGCGAGCACAGCUAGUUUUAGAGCUAGAAAUAGCAAGUUAAAAUAAGGCUAG | 18176 |
| 968 | P8R18_ins5_ | UCCGUUAUCAACUUGAAAAAGUGGCACCGAGUCGGUGCUCCGUGGCCUUAGUAACCCU | |
| | nick_v2 | GUGCUC | |
| RNACS7 | B2m2_ | GAGUAGCGCGAGCACAGCUAGUUUUAGAGCUAGAAAUAGCAAGUUAAAAUAAGGCUAG | 18177 |
| 969 | P13R20_ins5_ | UCCGUUAUCAACUUGAAAAAGUGGCACCGAGUCGGUGCGCUCCGUGGCCUUAGUAACC | |
| | nick_v2 | CUGUGCUCGCGCU | |
| RNACS7 | B2m2_ | GAGUAGCGCGAGCACAGCUAGUUUUAGAGCUAGAAAUAGCAAGUUAAAAUAAGGCUAG | 18178 |
| 970 | P12R20_ins5_ | UCCGUUAUCAACUUGAAAAAGUGGCACCGAGUCGGUGCGCUCCGUGGCCUUAGUAACC | |
| | nick_v2 | CUGUGCUCGCGC | |
| RNACS7 | B2m2_ | GAGUAGCGCGAGCACAGCUAGUUUUAGAGCUAGAAAUAGCAAGUUAAAAUAAGGCUAG | 18179 |
| 971 | P11R20_ins5_ | UCCGUUAUCAACUUGAAAAAGUGGCACCGAGUCGGUGCGCUCCGUGGCCUUAGUAACC | |
| | nick_v2 | CUGUGCUCGCG | |
| RNACS7 | B2m2_ | GAGUAGCGCGAGCACAGCUAGUUUUAGAGCUAGAAAUAGCAAGUUAAAAUAAGGCUAG | 18180 |
| 972 | P10R20_ins5_ | UCCGUUAUCAACUUGAAAAAGUGGCACCGAGUCGGUGCGCUCCGUGGCCUUAGUAACC | |
| | nick_v2 | CUGUGCUCGC | |
| RNACS7 | B2m2_ | GAGUAGCGCGAGCACAGCUAGUUUUAGAGCUAGAAAUAGCAAGUUAAAAUAAGGCUAG | 18181 |
| 973 | P9R20_ins5_ | UCCGUUAUCAACUUGAAAAAGUGGCACCGAGUCGGUGCGCUCCGUGGCCUUAGUAACC | |
| | nick_v2 | CUGUGCUCG | |
| RNACS7 | B2m2_ | GAGUAGCGCGAGCACAGCUAGUUUUAGAGCUAGAAAUAGCAAGUUAAAAUAAGGCUAG | 18182 |
| 974 | P8R20_ins5_ | UCCGUUAUCAACUUGAAAAAGUGGCACCGAGUCGGUGCGCUCCGUGGCCUUAGUAACC | |
| | nick_v2 | CUGUGCUC | |
| RNACS7 | B2m2_ | GAGUAGCGCGAGCACAGCUAGUUUUAGAGCUAGAAAUAGCAAGUUAAAAUAAGGCUAG | 18183 |
| 975 | P13R22_ins5_ | UCCGUUAUCAACUUGAAAAAGUGGCACCGAGUCGGUGCUCGCUCCGUGGCCUUAGUAA | |
| | nick_v2 | CCCUGUGCUCGCGCU | |
| RNACS7 | B2m2_ | GAGUAGCGCGAGCACAGCUAGUUUUAGAGCUAGAAAUAGCAAGUUAAAAUAAGGCUAG | 18184 |
| 976 | P12R22_ins5_ | UCCGUUAUCAACUUGAAAAAGUGGCACCGAGUCGGUGCUCGCUCCGUGGCCUUAGUAA | |
| | nick_v2 | CCCUGUGCUCGCGC | |
| RNACS7 | B2m2_ | GAGUAGCGCGAGCACAGCUAGUUUUAGAGCUAGAAAUAGCAAGUUAAAAUAAGGCUAG | 18185 |
| 977 | P11R22_ins5_ | UCCGUUAUCAACUUGAAAAAGUGGCACCGAGUCGGUGCUCGCUCCGUGGCCUUAGUAA | |
| | nick_v2 | CCCUGUGCUCGCG | |
| RNACS7 | B2m2_ | GAGUAGCGCGAGCACAGCUAGUUUUAGAGCUAGAAAUAGCAAGUUAAAAUAAGGCUAG | 18186 |
| 978 | P10R22_ins5_ | UCCGUUAUCAACUUGAAAAAGUGGCACCGAGUCGGUGCUCGCUCCGUGGCCUUAGUAA | |
| | nick_v2 | CCCUGUGCUCGC | |
| RNACS7 | B2m2_ | GAGUAGCGCGAGCACAGCUAGUUUUAGAGCUAGAAAUAGCAAGUUAAAAUAAGGCUAG | 18187 |
| 979 | P9R22_ins5_ | UCCGUUAUCAACUUGAAAAAGUGGCACCGAGUCGGUGCUCGCUCCGUGGCCUUAGUAA | |
| | nick_v2 | CCCUGUGCUCG | |
| RNACS7 | B2m2_ | GAGUAGCGCGAGCACAGCUAGUUUUAGAGCUAGAAAUAGCAAGUUAAAAUAAGGCUAG | 18188 |
| 980 | P8R22_ins5_ | UCCGUUAUCAACUUGAAAAAGUGGCACCGAGUCGGUGCUCGCUCCGUGGCCUUAGUAA | |
| | nick_v2 | CCCUGUGCUC | |
| RNACS7 | B2m2_ | GAGUAGCGCGAGCACAGCUAGUUUUAGAGCUAGAAAUAGCAAGUUAAAAUAAGGCUAG | 18189 |
| 981 | P13R24_ins5_ | UCCGUUAUCAACUUGAAAAAGUGGCACCGAGUCGGUGCUCUCGCUCCGUGGCCUUAGU | |
| | nick_v2 | AACCCUGUGCUCGCGCU | |
| RNACS7 | B2m2_ | GAGUAGCGCGAGCACAGCUAGUUUUAGAGCUAGAAAUAGCAAGUUAAAAUAAGGCUAG | 18190 |
| 982 | P12R24_ins5_ | UCCGUUAUCAACUUGAAAAAGUGGCACCGAGUCGGUGCUCUCGCUCCGUGGCCUUAGU | |
| | nick_v2 | AACCCUGUGCUCGCGC | |
| RNACS7 | B2m2_ | GAGUAGCGCGAGCACAGCUAGUUUUAGAGCUAGAAAUAGCAAGUUAAAAUAAGGCUAG | 18191 |
| 983 | P11R24_ins5_ | UCCGUUAUCAACUUGAAAAAGUGGCACCGAGUCGGUGCUCUCGCUCCGUGGCCUUAGU | |
| | nick_v2 | AACCCUGUGCUCGCG | |
| RNACS7 | B2m2_ | GAGUAGCGCGAGCACAGCUAGUUUUAGAGCUAGAAAUAGCAAGUUAAAAUAAGGCUAG | 18192 |
| 984 | P10R24_ins5_ | UCCGUUAUCAACUUGAAAAAGUGGCACCGAGUCGGUGCUCUCGCUCCGUGGCCUUAGU | |
| | nick_v2 | AACCCUGUGCUCGC | |
| RNACS7 | B2m2_ | GAGUAGCGCGAGCACAGCUAGUUUUAGAGCUAGAAAUAGCAAGUUAAAAUAAGGCUAG | 18193 |
| 985 | P9R24_ins5_ | UCCGUUAUCAACUUGAAAAAGUGGCACCGAGUCGGUGCUCUCGCUCCGUGGCCUUAGU | |
| | nick_v2 | AACCCUGUGCUCG | |
| RNACS7 | B2m2_ | GAGUAGCGCGAGCACAGCUAGUUUUAGAGCUAGAAAUAGCAAGUUAAAAUAAGGCUAG | 18194 |
| 986 | P8R24_ins5_ | UCCGUUAUCAACUUGAAAAAGUGGCACCGAGUCGGUGCUCUCGCUCCGUGGCCUUAGU | |
| | nick_v2 | AACCCUGUGCUC | |

In some embodiments, the template RNA comprises a sequence of a gRNA spacer, gRNA scaffold, 5′RT template sequence, insert, and PBS sequence of Table 3B. In some embodiments, the template RNA comprises a sequence of a gRNA spacer, gRNA scaffold, 5′RT template sequence, insert, 3′RT template sequence, and PBS sequence of Table 3B.

TABLE 3B
Exemplary Template RNA sequences comprising B2M1 or B2M2 gRNA spacers
Table 3B provides components of template RNAs comprising the B2M1
gRNA spacer (acUcacgcUggaUagccUcc SEQ ID NO. 18195) or a B2M2 gRNA
spacer (gagUagcgcgagcacagcUa SEQ ID NO. 18196) for making an edit
within the B2M gene either at a nick site or a PAM site.
The sequences provided include gRNA spacers, gRNA scaffolds,
5′ RT template sequences, 5-nucleotide insert
sequences, optional 3′ RT template sequences, and PBS sequences.

| | | | SEQ | | SEQ | | SEQ | | | | SEQ |
|---|---|---|---|---|---|---|---|---|---|---|---|
| | Location | | ID | | ID | | ID | | 3′ | | ID |
| Name | of edit | Spacer | NO | Scaffold | NO | 5′ RT | NO | insert | RT | PBS | NO |
| B2M1 | at nick | acUcac | 18195 | GUUUUAGAGCU | 17515 | cgcUccgUgg | 18197 | NNNNN | | ggcUaUccag | 18199 |
| | | gcUgg | | AGAAAUAGCAA | | ccUUagcUg | | or | | cgUgagU | |
| | | aUagcc | | GUUAAAAUAAG | | UgcUcgcgcU | | NNNN | | | |
| | | Ucc | | GCUAGUCCGUU | | acUcUcUcU | | | | | |
| | | | | AUCAACUUGAA | | UUcUggccU | | | | | |
| | | | | AAAGUGGCACC | | gga | | | | | |
| | | | | GAGUCGGUGC | | | | | | | |
| B2M2 | at nick | gagUa | 18196 | GUUUUAGAGCU | 17515 | UccUgaagcU | 18198 | NNNNN | | cUgUgcUcgc | 18200 |
| | | gcgcga | | AGAAAUAGCAA | | gacagcaUUc | | or | | gcUacUc | |
| | | gcacag | | GUUAAAAUAAG | | gggccgagaUg | | NNNN | | | |
| | | cUa | | GCUAGUCCGUU | | UcUcgcUccg | | | | | |
| | | | | AUCAACUUGAA | | UggccUUag | | | | | |
| | | | | AAAGUGGCACC | | | | | | | |
| | | | | GAGUCGGUGC | | | | | | | |

The sequences within Table 3B provide the suitable versions of the RT template sequence and PBS sequences for use in the template RNA. Other suitable versions may comprise shorter sequences compared to the sequences shown in Table 3B. In some embodiments, the 5′ RT template sequence can be varied from 0 to 47 nt for an “at PAM” edit, and 0 to 50 for an “at nick” edit. Consequently, in some embodiments, a template RNA described herein comprises a 5′ RT sequence of Table 3B or a portion thereof (e.g., a 3′ portion of the 5′ RT sequence). In some embodiments, where the 5′ RT template sequences comprises fewer bases than shown in Table 3B, bases are removed from the 5′ end. The insert sequences in Table 3B are shown simply with “N” placeholders to signify the ability to use any combination of 5 nucleotides within the template RNA. By way of example, Table 4B provides exemplary 5-nucleotide insertion sequences for use within a template RNA sequence described herein, e.g., the template RNA sequences of Table 3B. In some embodiments, the PBS sequence can be varied from 5 to 17 nucleotides. In some embodiments, where the PBS sequences comprises fewer bases than shown in Table 3B, bases are sequentially removed from the 3′ end. Consequently, in some embodiments, a template RNA described herein comprises a PBS sequence of Table 3B or a portion thereof (e.g., a 5′ portion of the PBS sequence).

TABLE 4B
Exemplary Insert sequences
Table 4B provides exemplary 4 or 5 nucleotide insert sequences
for use with template RNAs described herein.

| | 4 nucleotide |
|---|---|
| 5 nucleotide insertions | insertions |
| AAAAA AAAGA AAAAC AAAGC AAAAG AAAGG AAAAT AAAGT | AAAA AAAG |
| CAAAA CAAGA CAAAC CAAGC CAAAG CAAGG CAAAT CAAGT | CAAA CAAG |
| GAAAA GAAGA GAAAC GAAGC GAAAG GAAGG GAAAT GAAGT | GAAA GAAG |
| TAAAA TAAGA TAAAC TAAGC TAAAG TAAGG TAAAT TAAGT | TAAA TAAG |
| ACAAA ACAGA ACAAC ACAGC ACAAG ACAGG ACAAT ACAGT | ACAA ACAG |
| CCAAA CCAGA CCAAC CCAGC CCAAG CCAGG CCAAT CCAGT | CCAA CCAG |
| GCAAA GCAGA GCAAC GCAGC GCAAG GCAGG GCAAT GCAGT | GCAA GCAG |
| TCAAA TCAGA TCAAC TCAGC TCAAG TCAGG TCAAT TCAGT | TCAA TCAG |
| AGAAA AGAGA AGAAC AGAGC AGAAG AGAGG AGAAT AGAGT | AGAA AGAG |
| CGAAA CGAGA CGAAC CGAGC CGAAG CGAGG CGAAT CGAGT | CGAA CGAG |
| GGAAA GGAGA GGAAC GGAGC GGAAG GGAGG GGAAT GGAGT | GGAA GGAG |
| TGAAA TGAGA TGAAC TGAGC TGAAG TGAGG TGAAT TGAGT | TGAA TGAG |
| ATAAA ATAGA ATAAC ATAGC ATAAG ATAGG ATAAT ATAGT | ATAA ATAG |
| CTAAA CTAGA CTAAC CTAGC CTAAG CTAGG CTAAT CTAGT | CTAA CTAG |
| GTAAA GTAGA GTAAC GTAGC GTAAG GTAGG GTAAT GTAGT | GTAA GTAG |
| TTAAA TTAGA TTAAC TTAGC TTAAG TTAGG TTAAT TTAGT | TTAA TTAG |
| AACAA AACGA AACAC AACGC AACAG AACGG AACAT AACGT | AACA AACG |
| CACAA CACGA CACAC CACGC CACAG CACGG CACAT CACGT | CACA CACG |
| GACAA GACGA GACAC GACGC GACAG GACGG GACAT GACGT | GACA GACG |
| TACAA TACGA TACAC TACGC TACAG TACGG TACAT TACGT | TACA TACG |
| ACCAA ACCGA ACCAC ACCGC ACCAG ACCGG ACCAT ACCGT | ACCA ACCG |
| CCCAA CCCGA CCCAC CCCGC CCCAG CCCGG CCCAT CCCGT | CCCA CCCG |
| GCCAA GCCGA GCCAC GCCGC GCCAG GCCGG GCCAT GCCGT | GCCA GCCG |
| TCCAA TCCGA TCCAC TCCGC TCCAG TCCGG TCCAT TCCGT | TCCA TCCG |
| AGCAA AGCGA AGCAC AGCGC AGCAG AGCGG AGCAT AGCGT | AGCA AGCG |
| CGCAA CGCGA CGCAC CGCGC CGCAG CGCGG CGCAT CGCGT | CGCA CGCG |
| GGCAA GGCGA GGCAC GGCGC GGCAG GGCGG GGCAT GGCGT | GGCA GGCG |
| TGCAA TGCGA TGCAC TGCGC TGCAG TGCGG TGCAT TGCGT | TGCA TGCG |
| ATCAA ATCGA ATCAC ATCGC ATCAG ATCGG ATCAT ATCGT | ATCA ATCG |
| CTCAA CTCGA CTCAC CTCGC CTCAG CTCGG CTCAT CTCGT | CTCA CTCG |
| GTCAA GTCGA GTCAC GTCGC GTCAG GTCGG GTCAT GTCGT | GTCA GTCG |
| TTCAA TTCGA TTCAC TTCGC TTCAG TTCGG TTCAT TTCGT | TTCA TTCG |
| AAGAA AAGGA AAGAC AAGGC AAGAG AAGGG AAGAT AAGGT | AAGA AAGG |
| CAGAA CAGGA CAGAC CAGGC CAGAG CAGGG CAGAT CAGGT | CAGA CAGG |
| GAGAA GAGGA GAGAC GAGGC GAGAG GAGGG GAGAT GAGGT | GAGA GAGG |
| TAGAA TAGGA TAGAC TAGGC TAGAG TAGGG TAGAT TAGGT | TAGA TAGG |
| ACGAA ACGGA ACGAC ACGGC ACGAG ACGGG ACGAT ACGGT | ACGA ACGG |
| CCGAA CCGGA CCGAC CCGGC CCGAG CCGGG CCGAT CCGGT | CCGA CCGG |
| GCGAA GCGGA GCGAC GCGGC GCGAG GCGGG GCGAT GCGGT | GCGA GCGG |
| TCGAA TCGGA TCGAC TCGGC TCGAG TCGGG TCGAT TCGGT | TCGA TCGG |
| AGGAA AGGGA AGGAC AGGGC AGGAG AGGGG AGGAT AGGGT | AGGA AGGG |
| CGGAA CGGGA CGGAC CGGGC CGGAG CGGGG CGGAT CGGGT | CGGA CGGG |
| GGGAA GGGGA GGGAC GGGGC GGGAG GGGGG GGGAT GGGGT | GGGA GGGG |
| TGGAA TGGGA TGGAC TGGGC TGGAG TGGGG TGGAT TGGGT | TGGA TGGG |
| ATGAA ATGGA ATGAC ATGGC ATGAG ATGGG ATGAT ATGGT | ATGA ATGG |
| CTGAA CTGGA CTGAC CTGGC CTGAG CTGGG CTGAT CTGGT | CTGA CTGG |
| GTGAA GTGGA GTGAC GTGGC GTGAG GTGGG GTGAT GTGGT | GTGA GTGG |
| TTGAA TTGGA TTGAC TTGGC TTGAG TTGGG TTGAT TTGGT | TTGA TTGG |
| AATAA AATGA AATAC AATGC AATAG AATGG AATAT AATGT | AATA AATG |
| CATAA CATGA CATAC CATGC CATAG CATGG CATAT CATGT | CATA CATG |
| GATAA GATGA GATAC GATGC GATAG GATGG GATAT GATGT | GATA GATG |
| TATAA TATGA TATAC TATGC TATAG TATGG TATAT TATGT | TATA TATG |
| ACTAA ACTGA ACTAC ACTGC ACTAG ACTGG ACTAT ACTGT | ACTA ACTG |
| CCTAA CCTGA CCTAC CCTGC CCTAG CCTGG CCTAT CCTGT | CCTA CCTG |
| GCTAA GCTGA GCTAC GCTGC GCTAG GCTGG GCTAT GCTGT | GCTA GCTG |
| TCTAA TCTGA TCTAC TCTGC TCTAG TCTGG TCTAT TCTGT | TCTA TCTG |
| AGTAA AGTGA AGTAC AGTGC AGTAG AGTGG AGTAT AGTGT | AGTA AGTG |
| CGTAA CGTGA CGTAC CGTGC CGTAG CGTGG CGTAT CGTGT | CGTA CGTG |
| GGTAA GGTGA GGTAC GGTGC GGTAG GGTGG GGTAT GGTGT | GGTA GGTG |
| TGTAA TGTGA TGTAC TGTGC TGTAG TGTGG TGTAT TGTGT | TGTA TGTG |
| ATTAA ATTGA ATTAC ATTGC ATTAG ATTGG ATTAT ATTGT | ATTA ATTG |
| CTTAA CTTGA CTTAC CTTGC CTTAG CTTGG CTTAT CTTGT | CTTA CTTG |
| GTTAA GTTGA GTTAC GTTGC GTTAG GTTGG GTTAT GTTGT | GTTA GTTG |
| TTTAA TTTGA TTTAC TTTGC TTTAG TTTGG TTTAT TTTGT | TTTA TTTG |
| AAACA AAATA AAACC AAATC AAACG AAATG AAACT AAATT | AAAC AAAT |
| CAACA CAATA CAACC CAATC CAACG CAATG CAACT CAATT | CAAC CAAT |
| GAACA GAATA GAACC GAATC GAACG GAATG GAACT GAATT | GAAC GAAT |
| TAACA TAATA TAACC TAATC TAACG TAATG TAACT TAATT | TAAC TAAT |
| ACACA ACATA ACACC ACATC ACACG ACATG ACACT ACATT | ACAC ACAT |
| CCACA CCATA CCACC CCATC CCACG CCATG CCACT CCATT | CCAC CCAT |
| GCACA GCATA GCACC GCATC GCACG GCATG GCACT GCATT | GCAC GCAT |
| TCACA TCATA TCACC TCATC TCACG TCATG TCACT TCATT | TCAC TCAT |
| AGACA AGATA AGACC AGATC AGACG AGATG AGACT AGATT | AGAC AGAT |
| CGACA CGATA CGACC CGATC CGACG CGATG CGACT CGATT | CGAC CGAT |
| GGACA GGATA GGACC GGATC GGACG GGATG GGACT GGATT | GGAC GGAT |
| TGACA TGATA TGACC TGATC TGACG TGATG TGACT TGATT | TGAC TGAT |
| ATACA ATATA ATACC ATATC ATACG ATATG ATACT ATATT | ATAC ATAT |
| CTACA CTATA CTACC CTATC CTACG CTATG CTACT CTATT | CTAC CTAT |
| GTACA GTATA GTACC GTATC GTACG GTATG GTACT GTATT | GTAC GTAT |
| TTACA TTATA TTACC TTATC TTACG TTATG TTACT TTATT | TTAC TTAT |
| AACCA AACTA AACCC AACTC AACCG AACTG AACCT AACTT | AACC AACT |
| CACCA CACTA CACCC CACTC CACCG CACTG CACCT CACTT | CACC CACT |
| GACCA GACTA GACCC GACTC GACCG GACTG GACCT GACTT | GACC GACT |
| TACCA TACTA TACCC TACTC TACCG TACTG TACCT TACTT | TACC TACT |
| ACCCA ACCTA ACCCC ACCTC ACCCG ACCTG ACCCT ACCTT | ACCC ACCT |
| CCCCA CCCTA CCCCC CCCTC CCCCG CCCTG CCCCT CCCTT | CCCC CCCT |
| GCCCA GCCTA GCCCC GCCTC GCCCG GCCTG GCCCT GCCTT | GCCC GCCT |
| TCCCA TCCTA TCCCC TCCTC TCCCG TCCTG TCCCT TCCTT | TCCC TCCT |
| AGCCA AGCTA AGCCC AGCTC AGCCG AGCTG AGCCT AGCTT | AGCC AGCT |
| CGCCA CGCTA CGCCC CGCTC CGCCG CGCTG CGCCT CGCTT | CGCC CGCT |
| GGCCA GGCTA GGCCC GGCTC GGCCG GGCTG GGCCT GGCTT | GGCC GGCT |
| TGCCA TGCTA TGCCC TGCTC TGCCG TGCTG TGCCT TGCTT | TGCC TGCT |
| ATCCA ATCTA ATCCC ATCTC ATCCG ATCTG ATCCT ATCTT | ATCC ATCT |
| CTCCA CTCTA CTCCC CTCTC CTCCG CTCTG CTCCT CTCTT | CTCC CTCT |
| GTCCA GTCTA GTCCC GTCTC GTCCG GTCTG GTCCT GTCTT | GTCC GTCT |
| TTCCA TTCTA TTCCC TTCTC TTCCG TTCTG TTCCT TTCTT | TTCC TTCT |
| AAGCA AAGTA AAGCC AAGTC AAGCG AAGTG AAGCT AAGTT | AAGC AAGT |
| CAGCA CAGTA CAGCC CAGTC CAGCG CAGTG CAGCT CAGTT | CAGC CAGT |
| GAGCA GAGTA GAGCC GAGTC GAGCG GAGTG GAGCT GAGTT | GAGC GAGT |
| TAGCA TAGTA TAGCC TAGTC TAGCG TAGTG TAGCT TAGTT | TAGC TAGT |
| ACGCA ACGTA ACGCC ACGTC ACGCG ACGTG ACGCT ACGTT | ACGC ACGT |
| CCGCA CCGTA CCGCC CCGTC CCGCG CCGTG CCGCT CCGTT | CCGC CCGT |
| GCGCA GCGTA GCGCC GCGTC GCGCG GCGTG GCGCT GCGTT | GCGC GCGT |
| TCGCA TCGTA TCGCC TCGTC TCGCG TCGTG TCGCT TCGTT | TCGC TCGT |
| AGGCA AGGTA AGGCC AGGTC AGGCG AGGTG AGGCT AGGTT | AGGC AGGT |
| CGGCA CGGTA CGGCC CGGTC CGGCG CGGTG CGGCT CGGTT | CGGC CGGT |
| GGGCA GGGTA GGGCC GGGTC GGGCG GGGTG GGGCT GGGTT | GGGC GGGT |
| TGGCA TGGTA TGGCC TGGTC TGGCG TGGTG TGGCT TGGTT | TGGC TGGT |
| ATGCA ATGTA ATGCC ATGTC ATGCG ATGTG ATGCT ATGTT | ATGC ATGT |
| CTGCA CTGTA CTGCC CTGTC CTGCG CTGTG CTGCT CTGTT | CTGC CTGT |
| GTGCA GTGTA GTGCC GTGTC GTGCG GTGTG GTGCT GTGTT | GTGC GTGT |
| TTGCA TTGTA TTGCC TTGTC TTGCG TTGTG TTGCT TTGTT | TTGC TTGT |
| AATCA AATTA AATCC AATTC AATCG AATTG AATCT AATTT | AATC AATT |
| CATCA CATTA CATCC CATTC CATCG CATTG CATCT CATTT | CATC CATT |
| GATCA GATTA GATCC GATTC GATCG GATTG GATCT GATTT | GATC GATT |
| TATCA TATTA TATCC TATTC TATCG TATTG TATCT TATTT | TATC TATT |
| ACTCA ACTTA ACTCC ACTTC ACTCG ACTTG ACTCT ACTTT | ACTC ACTT |
| CCTCA CCTTA CCTCC CCTTC CCTCG CCTTG CCTCT CCTTT | CCTC CCTT |
| GCTCA GCTTA GCTCC GCTTC GCTCG GCTTG GCTCT GCTTT | GCTC GCTT |
| TCTCA TCTTA TCTCC TCTTC TCTCG TCTTG TCTCT TCTTT | TCTC TCTT |
| AGTCA AGTTA AGTCC AGTTC AGTCG AGTTG AGTCT AGTTT | AGTC AGTT |
| CGTCA CGTTA CGTCC CGTTC CGTCG CGTTG CGTCT CGTTT | CGTC CGTT |
| GGTCA GGTTA GGTCC GGTTC GGTCG GGTTG GGTCT GGTTT | GGTC GGTT |
| TGTCA TGTTA TGTCC TGTTC TGTCG TGTTG TGTCT TGTTT | TGTC TGTT |
| ATTCA ATTTA ATTCC ATTTC ATTCG ATTTG ATTCT ATTTT | ATTC ATTT |
| CTTCA CTTTA CTTCC CTTTC CTTCG CTTTG CTTCT CTTTT | CTTC CTTT |
| GTTCA GTTTA GTTCC GTTTC GTTCG GTTTG GTTCT GTTTT | GTTC GTTT |
| TTTCA TTTTA TTTCC TTTTC TTTCG TTTTG TTTCT TTTTT | TTTC TTTT |

Herein, when an RNA sequence (e.g., a template RNA sequence) is said to comprise a particular sequence that comprises thymine (T), it is of course understood that the RNA sequence may (and frequently does) comprise uracil (U) in place of T. For instance, the RNA sequence may comprise U at every position shown as T in the sequence disclosed herein.

In some embodiments of the systems and methods herein, the template RNA comprises a gRNA scaffold (e.g., that binds a heterologous gene modifying polypeptide, e.g., a Cas polypeptide) that comprises a sequence of a gRNA scaffold of any of Tables 1A, 1B, 2A, 2B, 3A, 3B, or 12. In some embodiments, the gRNA scaffold comprises a sequence having at least 70%, 75%, 80%, 85%, 90%, 95%, 96%, 97%, 98%, or 99% identity to a gRNA scaffold of any of Tables 1A, 1B, 2A, 2B, 3A, 3B, or 12. In some embodiments, the gRNA scaffold comprises a sequence of a scaffold region of any of Tables 1A, 1B, 2A, 2B, 3A, 3B, or 12 that corresponds to the RT template sequence, the spacer sequence, or both, or a sequence having at least 70%, 75%, 80%, 85%, 90%, 95%, 96%, 97%, 98%, or 99% identity thereto.

In some embodiments of the systems and methods herein, the system further comprises a second strand-targeting gRNA that directs a nick to the second strand of the human TRAC gene or the human B2M gene. In some embodiments, the second nick gRNA comprises a gRNA scaffold sequence that is orthogonal to the Cas domain of the heterologous gene modifying polypeptide. In some embodiments, the second nick gRNA comprises a gRNA scaffold sequence of Table 12.

The template RNA sequences shown in Tables 1A-3B may be customized depending on the cell being targeted. For example, in some embodiments it is desired to inactivate a PAM sequence upon editing (e.g., using a “PAM-kill” modification) to decrease the potential for further gene editing (e.g., by Cas retargeting) following the initial edit. Consequently, certain template RNAs described herein are designed to write a mutation (e.g., a substitution) into the PAM of the target site, such that upon editing, the PAM site will be mutated to a sequence no longer recognized by the heterologous gene modifying polypeptide. Thus, a mutation region within the heterologous object sequence of the template RNA may comprise a PAM-kill sequence. Without wishing to be bound by theory, in some embodiments, a PAM-kill sequence prevents re-engagement of the heterologous gene modifying polypeptide upon completion of a gene modification, or decreases re-engagement relative to a template RNA lacking a PAM-kill sequence. In some embodiments, a PAM-kill sequence does not alter the amino acid sequence encoded by a gene, e.g., the PAM-kill sequence results in a silent mutation. In other embodiments, it is desired to leave the PAM sequence intact (no PAM-kill).

Similarly, in some embodiments, to decrease the potential for further gene editing (e.g., by Cas retargeting) following the initial edit, it may be desirable to alter the first three nucleotides of the RT template sequence via a “seed-kill” motif. Consequently, certain template RNAs described herein are designed to write a mutation (e.g., a substitution) into the portion of the target site corresponding to the first three nucleotides of the RT template sequence, such that upon editing, the target site will be mutated to a sequence with lower homology to the RT template sequence. Thus, a mutation region within the heterologous object sequence of the template RNA may comprise a seed-kill sequence. Without wishing to be bound by theory, in some embodiments, a seed-kill sequence prevents re-engagement of the heterologous gene modifying polypeptide upon completion of genetic modification, or decreases re-engagement relative to an otherwise similar template RNA lacking a seed-kill sequence. In some embodiments, a seed-kill sequence does not alter the amino acid sequence encoded by a gene, e.g., the seed-kill sequence results in a silent mutation. In other embodiments, it is desired to leave the seed region intact, and a seed-kill sequence is not used.

In further embodiments, to optimize or improve gene editing efficiency, it may be desirable to evade the target cell's mismatch repair or nucleotide repair pathways or to bias the target cell's repair pathways toward preservation of the edited strand. In some embodiments, multiple silent mutations (for example, silent substitutions) may be introduced within the RT template sequence to evade the target cell's mismatch repair or nucleotide repair pathways or to bias the target cell's repair pathways toward preservation of the edited strand.

In some embodiments, the template RNA comprises one or more silent mutations.

It should be understood that the silent mutations may be used individually or combined in any manner in a template RNA sequence described herein.

In some embodiments, the template RNA comprises a sequence having one or more silent substitutions.

gRNAs with Inducible Activity

In some embodiments, a gRNA described herein (e.g., a gRNA that is part of a template RNA or a gRNA used for second strand nicking) has inducible activity. Inducible activity may be achieved by the template nucleic acid, e.g., template RNA, further comprising (in addition to the gRNA) a blocking domain, wherein the sequence of a portion of or all of the blocking domain is at least partially complementary to a portion or all of the gRNA. The blocking domain is thus capable of hybridizing or substantially hybridizing to a portion of or all of the gRNA. In some embodiments, the blocking domain and inducibly active gRNA are disposed on the template nucleic acid, e.g., template RNA, such that the gRNA can adopt a first conformation where the blocking domain is hybridized or substantially hybridized to the gRNA, and a second conformation where the blocking domain is not hybridized or not substantially hybridized to the gRNA. In some embodiments, in the first conformation the gRNA is unable to bind to the heterologous gene modifying polypeptide (e.g., the template nucleic acid binding domain, DNA binding domain, or endonuclease domain (e.g., a CRISPR/Cas protein)) or binds with substantially decreased affinity compared to an otherwise similar template RNA lacking the blocking domain. In some embodiments, in the second conformation the gRNA is able to bind to the heterologous gene modifying polypeptide (e.g., the template nucleic acid binding domain, DNA binding domain, or endonuclease domain (e.g., a CRISPR/Cas protein)). In some embodiments, whether the gRNA is in the first or second conformation can influence whether the DNA binding or endonuclease activities of the heterologous gene modifying polypeptide (e.g., of the CRISPR/Cas protein the heterologous gene modifying polypeptide comprises) are active.

In some embodiments, the gRNA that coordinates the second nick has inducible activity. In some embodiments, the gRNA that coordinates the second nick is induced after the template is reverse transcribed. In some embodiments, hybridization of the gRNA to the blocking domain can be disrupted using an opener molecule. In some embodiments, an opener molecule comprises an agent that binds to a portion or all of the gRNA or blocking domain and inhibits hybridization of the gRNA to the blocking domain. In some embodiments, the opener molecule comprises a nucleic acid, e.g., comprising a sequence that is partially or wholly complementary to the gRNA, blocking domain, or both. By choosing or designing an appropriate opener molecule, providing the opener molecule can promote a change in the conformation of the gRNA such that it can associate with a CRISPR/Cas protein and provide the associated functions of the CRISPR/Cas protein (e.g., DNA binding and/or endonuclease activity). Without wishing to be bound by theory, providing the opener molecule at a selected time and/or location may allow for spatial and temporal control of the activity of the gRNA, CRISPR/Cas protein, or heterologous gene modifying system comprising the same. In some embodiments, the opener molecule is exogenous to the cell comprising the heterologous gene modifying polypeptide and or template nucleic acid. In some embodiments, the opener molecule comprises an endogenous agent (e.g., endogenous to the cell comprising the heterologous gene modifying polypeptide and or template nucleic acid comprising the gRNA and blocking domain). For example, an inducible gRNA, blocking domain, and opener molecule may be chosen such that the opener molecule is an endogenous agent expressed in a target cell or tissue, e.g., thereby ensuring activity of a heterologous gene modifying system in the target cell or tissue. As a further example, an inducible gRNA, blocking domain, and opener molecule may be chosen such that the opener molecule is absent or not substantially expressed in one or more non-target cells or tissues, e.g., thereby ensuring that activity of a heterologous gene modifying system does not occur or substantially occur in the one or more non-target cells or tissues, or occurs at a reduced level compared to a target cell or tissue. Exemplary blocking domains, opener molecules, and uses thereof are described in PCT App. Publication WO2020044039A1, which is incorporated herein by reference in its entirety. In some embodiments, the template nucleic acid, e.g., template RNA, may comprise one or more sequences or structures for binding by one or more components of a heterologous gene modifying polypeptide, e.g., by a reverse transcriptase or RNA binding domain, and a gRNA. In some embodiments, the gRNA facilitates interaction with the template nucleic acid binding domain (e.g., RNA binding domain) of the heterologous gene modifying polypeptide. In some embodiments, the gRNA directs the heterologous gene modifying polypeptide to the matching target sequence, e.g., in a target cell genome.

### Second Strand Nicking

In some embodiments, a heterologous gene modifying system described herein comprises a nickase activity (e.g., in the heterologous gene modifying polypeptide) that nicks the first strand, and a nickase activity (e.g., in a polypeptide separate from the heterologous gene modifying polypeptide) that nicks the second strand of target DNA. As discussed herein, without wishing to be bound by theory, nicking of the first strand of the target site DNA is thought to provide a 3′ OH that can be used by an RT domain to reverse transcribe a sequence of a template RNA, e.g., a heterologous object sequence. Without wishing to be bound by theory, it is thought that introducing an additional nick to the second strand may bias the cellular DNA repair machinery to adopt the heterologous object sequence-based sequence more frequently than the original genomic sequence. In some embodiments, the additional nick to the second strand is made by the same endonuclease domain (e.g., nickase domain) as the nick to the first strand. In some embodiments, the same heterologous gene modifying polypeptide performs both the nick to the first strand and the nick to the second strand. In some embodiments, the heterologous gene modifying polypeptide comprises a CRISPR/Cas domain and the additional nick to the second strand is directed by an additional nucleic acid, e.g., comprising a second gRNA directing the CRISPR/Cas domain to nick the second strand. In other embodiments, the additional second strand nick is made by a different endonuclease domain (e.g., nickase domain) than the nick to the first strand. In some embodiments, that different endonuclease domain is situated in an additional polypeptide (e.g., a system of the invention further comprises the additional polypeptide), separate from the heterologous gene modifying polypeptide. In some embodiments, the additional polypeptide comprises an endonuclease domain (e.g., nickase domain) described herein. In some embodiments, the additional polypeptide comprises a DNA binding domain, e.g., described herein.

It is contemplated herein that the position at which the second strand nick occurs relative to the first strand nick may influence the extent to which one or more of: desired gene modifying DNA modifications are obtained, undesired double-strand breaks (DSBs) occur, undesired insertions occur, or undesired deletions occur. Without wishing to be bound by theory, second strand nicking may occur in two general orientations: inward nicks and outward nicks.

In some embodiments, in the inward nick orientation, the RT domain polymerizes (e.g., using the template RNA (e.g., the heterologous object sequence)) away from the second strand nick. In some embodiments, in the inward nick orientation, the location of the nick to the first strand and the location of the nick to the second strand are positioned between the first PAM site and second PAM site (e.g., in a scenario wherein both nicks are made by a polypeptide (e.g., a heterologous gene modifying polypeptide) comprising a CRISPR/Cas domain). When there are two PAMs on the outside and two nicks on the inside, this inward nick orientation can also be referred to as “PAM-out”. In some embodiments, in the inward nick orientation, the location of the nick to the first strand and the location of the nick to the second strand are between the sites where the polypeptide and the additional polypeptide bind to the target DNA. In some embodiments, in the inward nick orientation, the location of the nick to the second strand is positioned between the binding sites of the polypeptide and additional polypeptide, and the nick to the first strand is also located between the binding sites of the polypeptide and additional polypeptide. In some embodiments, in the inward nick orientation, the location of the nick to the first strand and the location of the nick to the second strand are positioned between the PAM site and the binding site of the second polypeptide which is at a distance from the target site.

An example of a heterologous gene modifying system that provides an inward nick orientation comprises a heterologous gene modifying polypeptide comprising a CRISPR/Cas domain, a template RNA comprising a gRNA that directs nicking of the target site DNA on the first strand, and an additional nucleic acid comprising an additional gRNA that directs nicking at a site a distance from the location of the first nick, wherein the location of the first nick and the location of the second nick are between the PAM sites of the sites to which the two gRNAs direct the heterologous gene modifying polypeptide. As a further example, another heterologous gene modifying system that provides an inward nick orientation comprises a heterologous gene modifying polypeptide comprising a zinc finger molecule and a first nickase domain wherein the zinc finger molecule binds to the target DNA in a manner that directs the first nickase domain to nick the first strand of the target site; an additional polypeptide comprising a CRISPR/Cas domain, and an additional nucleic acid comprising a gRNA that directs the additional polypeptide to nick a site a distance from the target site DNA on the second strand, wherein the location of the first nick and the location of the second nick are between the PAM site and the site to which the zinc finger molecule binds. As a further example, another heterologous gene modifying system that provides an inward nick orientation comprises a heterologous gene modifying polypeptide comprising a zinc finger molecule and a first nickase domain wherein the zinc finger molecule binds to the target DNA in a manner that directs the first nickase domain to nick the first strand of the target site; an additional polypeptide comprising a TAL effector molecule and a second nickase domain wherein the TAL effector molecule binds to a site a distance from the target site in a manner that directs the additional polypeptide to nick the second strand, wherein the location of the first nick and the location of the second nick are between the site to which the TAL effector molecule binds and the site to which the zinc finger molecule binds.

In some embodiments, in the outward nick orientation, the RT domain polymerizes (e.g., using the template RNA (e.g., the heterologous object sequence)) toward the second strand nick. In some embodiments, in the outward nick orientation when both the first and second nicks are made by a polypeptide comprising a CRISPR/Cas domain (e.g., a heterologous gene modifying polypeptide), the first PAM site and second PAM site are positioned between the location of the nick to the first strand and the location of the nick to the second strand. When there are two PAMs on the inside and two nicks on the outside, this outward nick orientation also can be referred to as “PAM-in”. In some embodiments, in the outward nick orientation, the polypeptide (e.g., the heterologous gene modifying polypeptide) and the additional polypeptide bind to sites on the target DNA between the location of the nick to the first strand and the location of the nick to the second. In some embodiments, in the outward nick orientation, the location of the nick to the second strand is positioned on the opposite side of the binding sites of the polypeptide and additional polypeptide relative to the location of the nick to the first strand. In some embodiments, in the outward orientation, the PAM site and the binding site of the second polypeptide which is at a distance from the target site are positioned between the location of the nick to the first strand and the location of the nick to the second strand.

An example of a heterologous gene modifying system that provides an outward nick orientation comprises a heterologous gene modifying polypeptide comprising a CRISPR/Cas domain, a template RNA comprising a gRNA that directs nicking of the target site DNA on the first strand, and an additional nucleic acid comprising an additional gRNA that directs nicking at a site a distance from the location of the first nick, wherein the location of the first nick and the location of the second nick are outside of the PAM sites of the sites to which the two gRNAs direct the heterologous gene modifying polypeptide (i.e., the PAM sites are between the location of the first nick and the location of the second nick). As a further example, another heterologous gene modifying system that provides an outward nick orientation comprises a heterologous gene modifying polypeptide comprising a zinc finger molecule and a first nickase domain wherein the zinc finger molecule binds to the target DNA in a manner that directs the first nickase domain to nick the first strand of the target site; an additional polypeptide comprising a CRISPR/Cas domain, and an additional nucleic acid comprising a gRNA that directs the additional polypeptide to nick a site a distance from the target site DNA on the second strand, wherein the location of the first nick and the location of the second nick are outside the PAM site and the site to which the zinc finger molecule binds (i.e., the PAM site and the site to which the zinc finger molecule binds are between the location of the first nick and the location of the second nick). As a further example, another heterologous gene modifying system that provides an outward nick orientation comprises a heterologous gene modifying polypeptide comprising a zinc finger molecule and a first nickase domain wherein the zinc finger molecule binds to the target DNA in a manner that directs the first nickase domain to nick the first strand of the target site; an additional polypeptide comprising a TAL effector molecule and a second nickase domain wherein the TAL effector molecule binds to a site a distance from the target site in a manner that directs the additional polypeptide to nick the second strand, wherein the location of the first nick and the location of the second nick are outside the site to which the TAL effector molecule binds and the site to which the zinc finger molecule binds (i.e., the site to which the TAL effector molecule binds and the site to which the zinc finger molecule binds are between the location of the first nick and the location of the second nick).

Without wishing to be bound by theory, it is thought that, for heterologous gene modifying systems where a second strand nick is provided, an outward nick orientation is preferred in some embodiments. As is described herein, an inward nick may produce a higher number of double-strand breaks (DSBs) than an outward nick orientation. DSBs may be recognized by the DSB repair pathways in the nucleus of a cell, which can result in undesired insertions and deletions. An outward nick orientation may provide a decreased risk of DSB formation, and a corresponding lower amount of undesired insertions and deletions. In some embodiments, undesired insertions and deletions are insertions and deletions not encoded by the heterologous object sequence, e.g., an insertion or deletion produced by the double-strand break repair pathway unrelated to the modification encoded by the heterologous object sequence. In some embodiments, a desired gene modification comprises a change to the target DNA (e.g., a substitution, insertion, or deletion) encoded by the heterologous object sequence (e.g., and achieved by the gene modifying writing the heterologous object sequence into the target site). In some embodiments, the first strand nick and the second strand nick are in an outward orientation.

In addition, the distance between the first strand nick and second strand nick may influence the extent to which one or more of: desired heterologous gene modifying system DNA modifications are obtained, undesired double-strand breaks (DSBs) occur, undesired insertions occur, or undesired deletions occur. Without wishing to be bound by theory, it is thought the second strand nick benefit, the biasing of DNA repair toward incorporation of the heterologous object sequence into the target DNA, increases as the distance between the first strand nick and second strand nick decreases. However, it is thought that the risk of DSB formation also increases as the distance between the first strand nick and second strand nick decreases. Correspondingly, it is thought that the number of undesired insertions and/or deletions may increase as the distance between the first strand nick and second strand nick decreases. In some embodiments, the distance between the first strand nick and second strand nick is chosen to balance the benefit of biasing DNA repair toward incorporation of the heterologous object sequence into the target DNA and the risk of DSB formation and of undesired deletions and/or insertions. In some embodiments, a system where the first strand nick and the second strand nick are at least a threshold distance apart has an increased level of desired heterologous gene modifying system modification outcomes, a decreased level of undesired deletions, and/or a decreased level of undesired insertions relative to an otherwise similar inward nick orientation system where the first nick and the second nick are less than the a threshold distance apart. In some embodiments the threshold distance(s) is given below.

In some embodiments, the first nick and the second nick are at least 20, 25, 30, 35, 40, 45, 50, 55, 60, 65, 70, 75, 80, 85, 90, 95, 100, 110, 120, 130, 140, 150, 160, 170, 180, 190, or 200 nucleotides apart. In some embodiments, the first nick and the second nick are no more than 25, 30, 35, 40, 45, 50, 55, 60, 65, 70, 75, 80, 85, 90, 95, 100, 110, 120, 130, 140, 150, 160, 170, 180, 190, 200, or 250 nucleotides apart. In some embodiments, the first nick and the second nick are 20-200, 30-200, 40-200, 50-200, 60-200, 70-200, 80-200, 90-200, 100-200, 110-200, 120-200, 130-200, 140-200, 150-200, 160-200, 170-200, 180-200, 190-200, 20-190, 30-190, 40-190, 50-190, 60-190, 70-190, 80-190, 90-190, 100-190, 110-190, 120-190, 130-190, 140-190, 150-190, 160-190, 170-190, 180-190, 20-180, 30-180, 40-180, 50-180, 60-180, 70-180, 80-180, 90-180, 100-180, 110-180, 120-180, 130-180, 140-180, 150-180, 160-180, 170-180, 20-170, 30-170, 40-170, 50-170, 60-170, 70-170, 80-170, 90-170, 100-170, 110-170, 120-170, 130-170, 140-170, 150-170, 160-170, 20-160, 30-160, 40-160, 50-160, 60-160, 70-160, 80-160, 90-160, 100-160, 110-160, 120-160, 130-160, 140-160, 150-160, 20-150, 30-150, 40-150, 50-150, 60-150, 70-150, 80-150, 90-150, 100-150, 110-150, 120-150, 130-150, 140-150, 20-140, 30-140, 40-140, 50-140, 60-140, 70-140, 80-140, 90-140, 100-140, 110-140, 120-140, 130-140, 20-130, 30-130, 40-130, 50-130, 60-130, 70-130, 80-130, 90-130, 100-130, 110-130, 120-130, 20-120, 30-120, 40-120, 50-120, 60-120, 70-120, 80-120, 90-120, 100-120, 110-120, 20-110, 30-110, 40-110, 50-110, 60-110, 70-110, 80-110, 90-110, 100-110, 20-100, 30-100, 40-100, 50-100, 60-100, 70-100, 80-100, 90-100, 20-90, 30-90, 40-90, 50-90, 60-90, 70-90, 80-90, 20-80, 30-80, 40-80, 50-80, 60-80, 70-80, 20-70, 30-70, 40-70, 50-70, 60-70, 20-60, 30-60, 40-60, 50-60, 20-50, 30-50, 40-50, 20-40, 30-40, or 20-30 nucleotides apart. In some embodiments, the first nick and the second nick are 40-100 nucleotides apart.

Without wishing to be bound by theory, it is thought that, for heterologous gene modifying systems where a second strand nick is provided and an inward nick orientation is selected, increasing the distance between the first strand nick and second strand nick may be preferred. As is described herein, an inward nick orientation may produce a higher number of DSBs than an outward nick orientation, and may result in a higher amount of undesired insertions and deletions than an outward nick orientation, but increasing the distance between the nicks may mitigate that increase in DSBs, undesired deletions, and/or undesired insertions. In some embodiments, an inward nick orientation wherein the first nick and the second nick are at least a threshold distance apart has an increased level of desired heterologous gene modifying system modification outcomes, a decreased level of undesired deletions, and/or a decreased level of undesired insertions relative to an otherwise similar inward nick orientation system where the first nick and the second nick are less than the a threshold distance apart. In some embodiments the threshold distance is given below.

In some embodiments, the first strand nick and the second strand nick are in an inward orientation. In some embodiments, the first strand nick and the second strand nick are in an inward orientation and the first strand nick and second strand nick are at least 100, 110, 120, 130, 140, 150, 160, 170, 180, 190, 200, 220, 240, 260, 280, 300, 350, 400, 450, or 500 nucleotides apart, e.g., at least 100 nucleotides apart, (and optionally no more than 500, 400, 300, 200, 190, 180, 170, 160, 150, 140, 130, or 120 nucleotides apart). In some embodiments, the first strand nick and the second strand nick are in an inward orientation and the first strand nick and second strand nick are 100-200, 110-200, 120-200, 130-200, 140-200, 150-200, 160-200, 170-200, 180-200, 190-200, 100-190, 110-190, 120-190, 130-190, 140-190, 150-190, 160-190, 170-190, 180-190, 100-180, 110-180, 120-180, 130-180, 140-180, 150-180, 160-180, 170-180, 100-170, 110-170, 120-170, 130-170, 140-170, 150-170, 160-170, 100-160, 110-160, 120-160, 130-160, 140-160, 150-160, 100-150, 110-150, 120-150, 130-150, 140-150, 100-140, 110-140, 120-140, 130-140, 100-130, 110-130, 120-130, 100-120, 110-120, or 100-110 nucleotides apart.

### Target Nucleic Acid Site

In some embodiments, after gene modification, the target site surrounding the edited sequence contains a limited number of insertions or deletions, for example, in less than about 50% or 10% of editing events, e.g., as determined by long-read amplicon sequencing of the target site, e.g., as described in Karst et al. (2020) bioRxiv doi.org/10.1101/645903 (incorporated by reference herein in its entirety). In some embodiments, the target site does not show multiple consecutive editing events, e.g., head-to-tail or head-to-head duplications, e.g., as determined by long-read amplicon sequencing of the target site, e.g., as described in Karst et al. bioRxiv doi.org/10.1101/645903 (2020) (incorporated herein by reference in its entirety). In some embodiments, the target site contains an integrated sequence corresponding to the template RNA. In some embodiments, the target site does not contain insertions resulting from endogenous RNA in more than about 1% or 10% of events, e.g., as determined by long-read amplicon sequencing of the target site, e.g., as described in Karst et al. bioRxiv doi.org/10.1101/645903 (2020) (incorporated herein by reference in its entirety). In some embodiments, the target site contains the integrated sequence corresponding to the template RNA.

In certain aspects of the present invention, the host DNA-binding site integrated into by the heterologous gene modifying system can be in a gene, in an intron, in an exon, an ORF, outside of a coding region of any gene, in a regulatory region of a gene, or outside of a regulatory region of a gene. In other aspects, the polypeptide may bind to one or more than one host DNA sequence.

In some embodiments, a heterologous gene modifying system is used to edit a target locus in multiple alleles. In some embodiments, a heterologous gene modifying system is designed to edit a specific allele. For example, a heterologous gene modifying polypeptide may be directed to a specific sequence that is only present on one allele, e.g., comprises a template RNA with homology to a target allele, e.g., a gRNA or annealing domain, but not to a second cognate allele. In some embodiments, a heterologous gene modifying system can alter a haplotype-specific allele. In some embodiments, a gene modifying system that targets a specific allele preferentially targets that allele, e.g., has at least a 2, 4, 6, 8, or 10-fold preference for a target allele.

### Combined Systems for Installing Multiple Gene Modifications

In one aspect, a first heterologous gene modifying system, for modifying DNA to install a mutation (e.g., resulting in a correction or deletion of an aberrant gene) into a human gene, may be combined with an additional system used to insert a heterologous object sequence (e.g., a CAR) into the genome of a cell.

In some embodiments, two systems may be utilized to introduce multiple edits to the same cell. In some embodiments, the two systems may be introduced into cells simultaneously or separately. In some embodiments, editing efficiency when making short edits (e.g., editing of 5 nucleotides or fewer) is not affected by the co-delivery of a gene modifying system containing a retrotransposon gene modifying polypeptide (e.g., directing the insertion of a gene).

In some embodiments, editing efficiency when inserting whole genes using the retrotransposon gene modifying system is not affected by the co-delivery of another exemplary system containing a heterologous gene modifying polypeptide (e.g., directing a short edit).

In some embodiments, treating T cells with both the first and second systems results in high level of co-occurrence of the two editing events in the same cell. In some embodiments, no skewing in the distribution of the subpopulations or phenotypes of T cells is observed when comparing mock treated T cells, T cells edited by just the first gene modifying system, and T cells edited by both the first and second gene modifying systems.

In some embodiments, the additional system comprises: (A) a retrotransposon gene modifying polypeptide or a nucleic acid encoding the gene modifying polypeptide, wherein the gene modifying polypeptide comprises: (i) an endonuclease and/or DNA binding domain; and (ii) a reverse transcriptase (RT) domain, where (i) and (ii) are both derived from a retrotransposon (e.g., from the same retrotransposon or different retrotransposons); and (B) a template RNA (or DNA encoding the template RNA) comprising (i) a sequence that binds the polypeptide and (ii) a heterologous object sequence. A retrotransposon gene modifying polypeptide, in some embodiments, acts as a substantially autonomous protein machine capable of integrating a template nucleic acid sequence into a target DNA molecule (e.g., in a mammalian host cell, such as a genomic DNA molecule in the host cell), substantially without relying on host machinery. The heterologous object sequence may include, e.g., a coding sequence, a regulatory sequence, a gene expression unit.

### Retrotransposon Gene Modifying Polypeptides

Non-long terminal repeat (LTR) retrotransposons are a type of mobile genetic elements that are widespread in eukaryotic genomes. They include, for example, the apurinic/apyrimidinic endonuclease (APE)-type, the restriction enzyme-like endonuclease (RLE)-type, and the Penelope-like element (PLE)-type.

The APE class retrotransposons are comprised of two functional domains: an endonuclease/DNA binding domain, and a reverse transcriptase domain. Examples of APE-class retrotransposons can be found, for example, in Table 1 of PCT Application No. PCT/US2019/048607, incorporated herein by reference in its entirety, including the sequence listing and sequences referred to in Table 1 therein.

The RLE class are comprised of three functional domains: a DNA binding domain, a reverse transcription domain, and an endonuclease domain. Examples of RLE-class retrotransposons can be found, for example, in Table 2 of PCT Application No. PCT/US2019/048607, incorporated herein by reference in its entirety, including the sequence listing and sequences referred to in Table 2 therein.

The reverse transcriptase domain of non-LTR retrotransposon functions by binding an RNA sequence template and reverse transcribing it into the host genome's target DNA. The RNA sequence template has a 3′ untranslated region which is specifically bound to the retrotransposase, and a variable 5′ region generally having Open Reading Frame(s) (“ORF”) encoding retrotransposase proteins. The RNA sequence template may also comprise a 5′ untranslated region which specifically binds the retrotransposase.

Penelope-like elements (PLEs) are distinct from both LTR and non-LTR retrotransposons. PLEs generally comprise a reverse transcriptase domain distinct from that of APE and RLE elements, but similar to that of telomerases and Group II introns, and an optional GIY-YIG endonuclease domain.

Other exemplary classes of retrotransposon include, without limitation, RTE (e.g., RTE-1_MD, RTE-3_BF, and RTE-25_LMi), CR1 (e.g., CR1-1_PH), Crack (e.g., Crack-28_RF), L2 (e.g., L2-2_Dre and L2-5_GA), and Vingi (e.g., Vingi-1_Acar) retrotransposons.

As described herein, the elements of such retrotransposons can be functionally modularized and/or modified to target, edit, modify or manipulate a target DNA sequence, e.g., to insert an object (e.g., heterologous) nucleic acid sequence into a target genome, e.g., a mammalian genome, by reverse transcription. In some embodiments, a retrotransposon gene modifying system comprises: (A) a polypeptide or a nucleic acid encoding a polypeptide, wherein the polypeptide comprises (i) a retrotransposase reverse transcriptase domain, and (ii) a retrotransposase endonuclease domain that contains DNA binding functionality; and (B) a template RNA (or DNA encoding the template RNA) comprising (i) a sequence that binds the polypeptide and (ii) a heterologous object sequence. The RNA template element of a retrotransposon gene modifying system is typically heterologous to the polypeptide element and provides an object sequence to be inserted (reverse transcribed) into the host genome.

In some embodiments, the retrotransposon gene modifying system comprises a retrotransposase sequence of an element listed in any one of Table 10, Table 11, Table X, Table Z1 Table 3A, or 3B of PCT Pub. No.: WO/2021/178717, which are incorporated herein by reference as they relate to domains from retrotransposons.

In some embodiments, an amino acid sequence encoded by an element of Table R1 is an amino acid sequence encoded by the full length sequence of an element listed in Table R1, or a sequence having at least 75%, 80%, 85%, 90%, 95%, 96%, 97%, 98%, or 99% sequence identity thereto. In some embodiments, the full-length sequence of an element listed in Table R1 may comprise one or more (e.g., all of) of a 5′ UTR, polypeptide-encoding sequence, or 3′ UTR of a retrotransposon as described herein. In some embodiments, an amino acid sequence of Table R1 is an amino acid sequence encoded by the full length sequence of an element listed in Table R1, or a sequence having at least 75%, 80%, 85%, 90%, 95%, 96%, 97%, 98%, or 99% sequence identity thereto. In some embodiments, a 5′ UTR of an element of Table R1 comprises a 5′ UTR of the full length sequence of an element listed in Table R1, or a sequence having at least 75%, 80%, 85%, 90%, 95%, 96%, 97%, 98%, or 99% sequence identity thereto. In some embodiments, a 3′ UTR of an element of Table R1 comprises a 3′ UTR of the full length sequence of an element listed in Table R1, or a sequence having at least 75%, 80%, 85%, 90%, 95%, 96%, 97%, 98%, or 99% sequence identity thereto.

Also indicated in Table R1 are the host organisms from which the nucleic acid sequences were obtained and a listing of domains present within the polypeptide encoded by the open reading frame of the nucleic acid sequence.

In some embodiments, the retrotransposon gene modifying polypeptide comprises an amino acid sequence of SEQ ID NO: 400 or a sequence at least 70%, 75%, 80%, 85%, 90%, 95%, 96%, 97%, 98%, or 99% identical thereto, or a functional fragment thereof (e.g., having one or both of reverse transcriptase activity and endonuclease activity). In some embodiments, the retrotransposon gene modifying polypeptide comprises an amino acid sequence of SEQ ID NO: 400. In some embodiments, the sequence that binds the polypeptide comprises:
a 5′ UTR having a sequence of SEQ ID NO: 700 or a sequence at least 80%, 85%, 90%, 95%, 96%, 97%, 98%, or 99% identical thereto; and
a 3′ UTR having a sequence of SEQ ID NO: 800 or a sequence at least 80%, 85%, 90%, 95%, 96%, 97%, 98%, or 99% identical thereto.

In some embodiments, the retrotransposon gene modifying polypeptide comprises an amino acid sequence of SEQ ID NO: 401 or a sequence at least 70%, 75%, 80%, 85%, 90%, 95%, 96%, 97%, 98%, or 99% identical thereto, or a functional fragment thereof (e.g., having one or both of reverse transcriptase activity and endonuclease activity). In some embodiments, the retrotransposon gene modifying polypeptide comprises an amino acid sequence of SEQ ID NO: 401. In some embodiments, the sequence that binds the polypeptide comprises:
a 5′ UTR having a sequence of SEQ ID NO: 701 or a sequence at least 80%, 85%, 90%, 95%, 96%, 97%, 98%, or 99% identical thereto; and
a 3′ UTR having a sequence of SEQ ID NO: 801 or a sequence at least 80%, 85%, 90%, 95%, 96%, 97%, 98%, or 99% identical thereto.

In some embodiments, the retrotransposon gene modifying polypeptide comprises an amino acid sequence of SEQ ID NO: 402 or a sequence at least 70%, 75%, 80%, 85%, 90%, 95%, 96%, 97%, 98%, or 99% identical thereto, or a functional fragment thereof (e.g., having one or both of reverse transcriptase activity and endonuclease activity). In some embodiments, the retrotransposon gene modifying polypeptide comprises an amino acid sequence of SEQ ID NO: 402. In some embodiments, the sequence that binds the polypeptide comprises:
a 5′ UTR having a sequence of SEQ ID NO: 702 or a sequence at least 80%, 85%, 90%, 95%, 96%, 97%, 98%, or 99% identical thereto; and
a 3′ UTR having a sequence of SEQ ID NO: 802 or a sequence at least 80%, 85%, 90%, 95%, 96%, 97%, 98%, or 99% identical thereto.

In some embodiments, the retrotransposon gene modifying polypeptide comprises an amino acid sequence of SEQ ID NO: 403, or a sequence having at least 70%, 75%, 80%, 85%, 90%, 95%, 96%, 97%, 98%, or 99% identity thereto, wherein one or both of:
i) amino acid position 345 is other than D, e.g., is N, and
ii) amino acid position 523 is other than T, e.g., is S;
or a functional fragment thereof (e.g., having one or both of reverse transcriptase activity and endonuclease activity).

In some embodiments, the retrotransposon gene modifying polypeptide comprises an amino acid sequence of SEQ ID NO: 403, or a sequence having at least 70%, 75%, 80%, 85%, 90%, 95%, 96%, 97%, 98%, or 99% identity thereto, wherein one or both of:
i) amino acid position 345 is N, and
ii) amino acid position 523 is S;
or a functional fragment thereof (e.g., having one or both of reverse transcriptase activity and endonuclease activity).

In some embodiments, the retrotransposon gene modifying polypeptide comprises an amino acid sequence of SEQ ID NO: 403 or a sequence at least 70%, 75%, 80%, 85%, 90%, 95%, 96%, 97%, 98%, or 99% identical thereto, or a functional fragment thereof (e.g., having one or both of reverse transcriptase activity and endonuclease activity). In some embodiments, the retrotransposon gene modifying polypeptide comprises an amino acid sequence of SEQ ID NO: 403. In some embodiments, the sequence that binds the polypeptide comprises:
a 5′ UTR having a sequence of SEQ ID NO: 703 or a sequence at least 80%, 85%, 90%, 95%, 96%, 97%, 98%, or 99% identical thereto; and
a 3′ UTR having a sequence of SEQ ID NO: 803 or a sequence at least 80%, 85%, 90%, 95%, 96%, 97%, 98%, or 99% identical thereto.

In some embodiments, the retrotransposon gene modifying polypeptide comprises an amino acid sequence of SEQ ID NO: 404 or a sequence at least 70%, 75%, 80%, 85%, 90%, 95%, 96%, 97%, 98%, or 99% identical thereto, or a functional fragment thereof (e.g., having one or both of reverse transcriptase activity and endonuclease activity). In some embodiments, the retrotransposon gene modifying polypeptide comprises an amino acid sequence of SEQ ID NO: 404. In some embodiments, the sequence that binds the polypeptide comprises:
a 5′ UTR having a sequence of SEQ ID NO: 704 or a sequence at least 80%, 85%, 90%, 95%, 96%, 97%, 98%, or 99% identical thereto; and
a 3′ UTR having a sequence of SEQ ID NO: 805 or a sequence at least 80%, 85%, 90%, 95%, 96%, 97%, 98%, or 99% identical thereto.

In some embodiments, the retrotransposon gene modifying polypeptide comprising an amino acid sequence of SEQ ID NO: 405, or a sequence having at least 70%, 75%, 80%, 85%, 90%, 95%, 96%, 97%, 98%, or 99% identity thereto, wherein one, two, or three of:
i) amino acid position 444 is other than P, e.g., is A,
ii) amino acid position 848 is other than D, e.g., is G; and
iii) amino acid position 875 is other than T, e.g., is A
or a functional fragment thereof (e.g., having one or both of reverse transcriptase activity and endonuclease activity).

In some embodiments, the retrotransposon gene modifying polypeptide comprising an amino acid sequence of SEQ ID NO:405, or a sequence having at least 70%, 75%, 80%, 85%, 90%, 95%, 96%, 97%, 98%, or 99% identity thereto, wherein one, two, or three of:
i) amino acid position 444 is A,
ii) amino acid position 848 is G; and
iii) amino acid position 875 is A
or a functional fragment thereof (e.g., having one or both of reverse transcriptase activity and endonuclease activity).

In some embodiments, the retrotransposon gene modifying polypeptide comprises an amino acid sequence of SEQ ID NO: 405 or a sequence at least 70%, 75%, 80%, 85%, 90%, 95%, 96%, 97%, 98%, or 99% identical thereto, or a functional fragment thereof (e.g., having one or both of reverse transcriptase activity and endonuclease activity). In some embodiments, the retrotransposon gene modifying polypeptide comprises an amino acid sequence of SEQ ID NO: 405. In some embodiments, the sequence that binds the polypeptide comprises:
a 5′ UTR having a sequence of SEQ ID NO: 705 or a sequence at least 80%, 85%, 90%, 95%, 96%, 97%, 98%, or 99% identical thereto; and
a 3′ UTR having a sequence of SEQ ID NO: 805 or a sequence at least 80%, 85%, 90%, 95%, 96%, 97%, 98%, or 99% identical thereto.

In some embodiments, the retrotransposon gene modifying polypeptide comprises an amino acid sequence of SEQ ID NO: 406 or a sequence at least 70%, 75%, 80%, 85%, 90%, 95%, 96%, 97%, 98%, or 99% identical thereto, or a functional fragment thereof (e.g., having one or both of reverse transcriptase activity and endonuclease activity). In some embodiments, the retrotransposon gene modifying polypeptide comprises an amino acid sequence of SEQ ID NO: 406. In some embodiments, the sequence that binds the polypeptide comprises:
a 5′ UTR having a sequence of SEQ ID NO: 706 or a sequence at least 80%, 85%, 90%, 95%, 96%, 97%, 98%, or 99% identical thereto; and
a 3′ UTR having a sequence of SEQ ID NO: 806 or a sequence at least 80%, 85%, 90%, 95%, 96%, 97%, 98%, or 99% identical thereto.

In some embodiments, the retrotransposon gene modifying polypeptide comprises an amino acid sequence of SEQ ID NO: 407 or a sequence at least 70%, 75%, 80%, 85%, 90%, 95%, 96%, 97%, 98%, or 99% identical thereto, or a functional fragment thereof (e.g., having one or both of reverse transcriptase activity and endonuclease activity). In some embodiments, the retrotransposon gene modifying polypeptide comprises an amino acid sequence of SEQ ID NO: 407. In some embodiments, the sequence that binds the polypeptide comprises:
a 5′ UTR having a sequence of SEQ ID NO: 707 or a sequence at least 80%, 85%, 90%, 95%, 96%, 97%, 98%, or 99% identical thereto; and
a 3′ UTR having a sequence of SEQ ID NO: 807 or a sequence at least 80%, 85%, 90%, 95%, 96%, 97%, 98%, or 99% identical thereto.

In some embodiments, the retrotransposon gene modifying polypeptide comprises an amino acid sequence of SEQ ID NO: 408 or a sequence at least 70%, 75%, 80%, 85%, 90%, 95%, 96%, 97%, 98%, or 99% identical thereto, or a functional fragment thereof (e.g., having one or both of reverse transcriptase activity and endonuclease activity). In some embodiments, the retrotransposon gene modifying polypeptide comprises an amino acid sequence of SEQ ID NO: 408. In some embodiments, the sequence that binds the polypeptide comprises:
a 5′ UTR having a sequence of SEQ ID NO: 708 or a sequence at least 80%, 85%, 90%, 95%, 96%, 97%, 98%, or 99% identical thereto; and
a 3′ UTR having a sequence of SEQ ID NO: 808 or a sequence at least 80%, 85%, 90%, 95%, 96%, 97%, 98%, or 99% identical thereto.

In some embodiments, the retrotransposon gene modifying polypeptide comprises an amino acid sequence of SEQ ID NO: 409 or a sequence at least 70%, 75%, 80%, 85%, 90%, 95%, 96%, 97%, 98%, or 99% identical thereto, or a functional fragment thereof (e.g., having one or both of reverse transcriptase activity and endonuclease activity). In some embodiments, the retrotransposon gene modifying polypeptide comprises an amino acid sequence of SEQ ID NO: 409. In some embodiments, the sequence that binds the polypeptide comprises:
a 5′ UTR having a sequence of SEQ ID NO: 709 or a sequence at least 80%, 85%, 90%, 95%, 96%, 97%, 98%, or 99% identical thereto; and
a 3′ UTR having a sequence of SEQ ID NO: 809 or a sequence at least 80%, 85%, 90%, 95%, 96%, 97%, 98%, or 99% identical thereto.

In some embodiments, the retrotransposon gene modifying polypeptide comprising an amino acid sequence of SEQ ID NO: 410, or a sequence having at least 70%, 75%, 80%, 85%, 90%, 95%, 96%, 97%, 98%, or 99% identity thereto, wherein one or both of:
i) amino acid position 476 is a non-polar residue, e.g., is L, and
ii) amino acid position 524 is a non-polar residue, e.g., is L;
or a functional fragment thereof (e.g., having one or both of reverse transcriptase activity and endonuclease activity).

In some embodiments, the retrotransposon gene modifying polypeptide comprising an amino acid sequence of SEQ ID NO: 410, or a sequence having at least 70%, 75%, 80%, 85%, 90%, 95%, 96%, 97%, 98%, or 99% identity thereto, wherein one or both of:
i) amino acid position 476 is L, and
ii) amino acid position 524 is L;
or a functional fragment thereof (e.g., having one or both of reverse transcriptase activity and endonuclease activity).

In some embodiments, the retrotransposon gene modifying polypeptide comprises an amino acid sequence of SEQ ID NO: 410 or a sequence at least 70%, 75%, 80%, 85%, 90%, 95%, 96%, 97%, 98%, or 99% identical thereto, or a functional fragment thereof (e.g., having one or both of reverse transcriptase activity and endonuclease activity). In some embodiments, the retrotransposon gene modifying polypeptide comprises an amino acid sequence of SEQ ID NO: 410. In some embodiments, the sequence that binds the polypeptide comprises:
a 5′ UTR having a sequence of SEQ ID NO: 710 or a sequence at least 80%, 85%, 90%, 95%, 96%, 97%, 98%, or 99% identical thereto; and
a 3′ UTR having a sequence of SEQ ID NO: 810 or a sequence at least 80%, 85%, 90%, 95%, 96%, 97%, 98%, or 99% identical thereto.

In some embodiments, the retrotransposon gene modifying polypeptide comprises an amino acid sequence of SEQ ID NO: 420E, or a sequence having at least 70%, 75%, 80%, 85%, 90%, 95%, 96%, 97%, 98%, or 99% identity thereto, wherein amino acid position 191 is other than D, e.g., is A, or a fragment thereof having reverse transcriptase activity. In some embodiments, the retrotransposon gene modifying polypeptide comprises an amino acid sequence of SEQ ID NO: 420E, or a sequence having no more than 10, 20, 30, 40, 50, 60, 70, 80, 90, or 100 nucleotide differences thereto, wherein amino acid position 191 is other than D, e.g., is A, or a fragment thereof having reverse transcriptase activity. In some embodiments, the retrotransposon gene modifying polypeptide has an endonuclease activity of less than 20%, 15%, 10%, or 5% of that of a polypeptide of SEQ ID NO: 405 in an assay according to Example 4.

In certain embodiments, the retrotransposon gene modifying polypeptide further comprises a heterologous protein domain. In some embodiments, a linker (e.g., as described in Table 10 herein) is disposed between the heterologous protein domain and the amino acid sequence having at least 70%, 75%, 80%, 85%, 90%, 95%, 96%, 97%, 98%, or 99% identity to SEQ ID NO: 405, or fragment thereof.

In some embodiments, the retrotransposon gene modifying polypeptide comprises an amino acid sequence of SEQ ID NO: 421E, or a sequence having at least 70%, 75%, 80%, 85%, 90%, 95%, 96%, 97%, 98%, or 99% identity thereto, wherein amino acid position 191 is other than D, e.g., is A, or a fragment thereof having reverse transcriptase activity. In some embodiments, the retrotransposon gene modifying polypeptide comprises an amino acid sequence of SEQ ID NO: 421E, or a sequence having no more than 10, 20, 30, 40, 50, 60, 70, 80, 90, or 100 nucleotide differences thereto, wherein amino acid position 191 is other than D, e.g., is A, or a fragment thereof having reverse transcriptase activity. In some embodiments, the retrotransposon gene modifying polypeptide has an endonuclease activity of less than 20%, 15%, 10%, or 5% of that of a polypeptide of SEQ ID NO: 406.

In certain embodiments, the retrotransposon gene modifying polypeptide further comprises a heterologous protein domain. In some embodiments, a linker (e.g., as described in Table 10 herein) is disposed between the heterologous protein domain and the amino acid sequence having at least 70%, 75%, 80%, 85%, 90%, 95%, 96%, 97%, 98%, or 99% identity to SEQ ID NO: 406, or fragment thereof.

Table R1 provides retrotransposon gene modifying polypeptides comprising retrotransposon elements, altered for improved efficiency of integration into the human genome. Retrotransposase polypeptides were improved through consensus mapping to re-derive the optimal amino acid sequence. Template molecules for use with cognate retrotransposase enzymes were mapped back to their host genomes and flanking genomic DNA used to elucidate target site motifs. When detectable, conserved sequence motifs from the flanking genomic DNA of endogenous occurrences of an element were aligned to the human genome, and new sequences were derived from the human genome as 5′ or 3′ “Human Homology Arms.” In some embodiments, a template RNA described herein comprises one or both of a first homology domain comprising a sequence of a 5′ Human Homology Arm of Table R1 (or a sequence having at least 80%, 85%, 90%, 95%, 96%, 97%, 98%, or 99% identity thereto) and a second homology domain comprising a sequence of a 3′ Human Homology Arm of Table R1 (or a sequence having at least 80%, 85%, 90%, 95%, 96%, 97%, 98%, or 99% identity thereto).

TABLE R1
Retrotransposase systems with improved integration activity

| | | | | | 5′ Human | 3′ Human | | |
|---|---|---|---|---|---|---|---|---|
| | | | Target | Consensus Optimized Protein | Homology | Homology | | |
| Element | Organism | Domains | Motif | Sequence | Arm | Arm | 5′ UTR | 3′ UTR |
| L2-2_DRe | Danio | RT and | TGAG | (SEQ ID NO: 400) | (SEQ ID | a | (SEQ ID NO: | (SEQ ID |
| (1) | rerio | EN | CACG | MCFLIPVVTNTRKTREVRCRRN | NO: 500) | | 700) | NO: 800) |
| | | | GTAG | PHNLRSIHVSTISQLSLSVGLWN | GTTTCGC | | GCAGGAGAA | TAATCT |
| | | | CATTA | CQSAVNKADFITSIATYSDYNL | GTCGTCC | | GCACTTTAG | GCAATT |
| | | | GTCGa | MALTETWLRPEDTATHATLSA | GCCTCA | | CAGCATCTA | GCCTCT |
| | | | (SEQ | NFSFSHTPRQTGRGGGTGLLISK | GTTTCGG | | GAACAGCAG | CTGAAT |
| | | | ID NO: | EWKFTLIPSLPTISSFEFHAVTIIH | CCCTTGT | | CCTGTAAGT | ATCACA |
| | | | 37657) | PFYINVVVIYRPPGKLGHFLDEL | TTTGACT | | ACATTTAAG | CTAACT |
| | | | | DVLLSSFSNFATPLLVLGDFNIY | CGGGAG | | ATTTGTTTCA | GTACCC |
| | | | | VDKPQAADFQTLLASFDLKRAP | GCGTGTC | | GTTGTTGTGT | AAAAA |
| | | | | TSATHKSGNQLDLIYTRHCFTD | CAAACG | | ATGGTTTGA | AAAAA |
| | | | | QTIVTPLQISDHFLLSLNIHITPEP | CCAGGT | | GGACTTGTT | AAAAA |
| | | | | PHTPTLVTFRRNLRSLSPNRLSTI | AACCAC | | TCCAGCTGT | AAAAA |
| | | | | VSDSLPPSRKLTALDSNSATNTL | TATATAG | | TTGTGTAAA | AAAAA |
| | | | | CSTLASCLDRLCPLASRPARASP | TGAGCA | | GTTGTAGGA | ATAAAA |
| | | | | PAPWLSDALREHRSKLRAAERI | CGGTAG | | CATTTAAAC | AWACT |
| | | | | WRKTKNPAHLLTYQTLLSSFSA | CATTAGT | | TTGCTTTCAG | ACTAAT |
| | | | | EVTSAKQTYYRLKINNATNPRL | CG | | TTGTGTATA | ACTTCC |
| | | | | LFKTFSSLLYPPPPPASSTLTTDD | | | ATACTAGAA | CTTCTT |
| | | | | FATFFCTKTAKISAQFAAPTTNT | | | GTTGTTTAG | AGACTT |
| | | | | QDTTPTPHTLTSFSQLSESEVSK | | | CCACTGTTTC | TACAGA |
| | | | | LVLSSHATTCPLDPIPSHLLQAIS | | | CTTGGTTACT | CCTGAA |
| | | | | PAVIPTLTHIINTSLDSGLFPTTF | | | ATAAGAGCT | ACTTGC |
| | | | | KQARVTPLLKKPNLDHTLLENY | | | TGTGTAGCG | CTATAG |
| | | | | RPVSLLPFMAKILEKVVFNQVL | | | AACGCAGAC | CACTTA |
| | | | | DFLTQNNLMDNKQSGFKKGHS | | | GCGGTTCGC | TTCATT |
| | | | | TETALLSVVEDLRLAKADSKSS | | | GTCGTCCGC | GTTGCT |
| | | | | VLILLDLSAAFDTVNHQILLSTL | | | CTCAGTTTC | CTTAGT |
| | | | | ESLGVAGTVIQWFRSYLSDRSF | | | GGCCCTTGT | TGTGTA |
| | | | | RVSWRGEVSNLQHLNTGVPQG | | | TTTGACTCG | AATTGC |
| | | | | SVLGPLLFSIYTSSLGPVIQRHGF | | | GGAGGCGTG | TTCCTT |
| | | | | SYHCYADDTQLYLSFHPDDPSV | | | TCCAAACGC | GTCCTC |
| | | | | PARISACLLDISHWMKDHHLQL | | | CAGGTAACC | ATTTGT |
| | | | | NLAKTEMLVVSANPTLHHNFSI | | | ACTATATAG | AAGTCG |
| | | | | QMDGATITASKMVKSLGVTIDD | | | TGAGCACGG | CTTTGG |
| | | | | QLNFSDHISRTARSCRFALYNIR | | | TAGCATTAG | ATAAAA |
| | | | | KIRPFLSEHAAQLLVQALVLSK | | | TCGGCAGGA | GCGTCT |
| | | | | LDYCNSLLAGLPANSIKPLQLL | | | GAAGCACTT | GCTAAA |
| | | | | QNAAARVVFNEPKRAHVTPLL | | | TAGCAGCAT | TGACTA |
| | | | | VRLHWLPVAARIKFKTLMFAY | | | CTAGAACAG | AATGTA |
| | | | | KVTSGLAPSYLHSLLQIYVPSRN | | | CAGCCTGTA | AATGTA |
| | | | | LRSVNERRLVVPSQRGKKSLSR | | | AGTACATTT | AATGT |
| | | | | TLTLNLPSWWNELPNCIRTAES | | | AAGATTTGT | |
| | | | | LAIFKKRLKTQLFSLHFTS | | | TTCAGTTGTT | |
| | | | | | | | GTGTATGGT | |
| | | | | | | | TTGAGGACT | |
| | | | | | | | TGTTTCCAG | |
| | | | | | | | CTGTTTGTGT | |
| | | | | | | | AAAGTTGTA | |
| | | | | | | | GGACATTTA | |
| | | | | | | | AACTTGCTTT | |
| | | | | | | | CAGTTGTGT | |
| | | | | | | | ATAATACTA | |
| | | | | | | | GAAGTTGTT | |
| | | | | | | | TAGCCACTG | |
| | | | | | | | TTTCCTTGGT | |
| | | | | | | | TACTATAAG | |
| | | | | | | | AGCTTGTGT | |
| | | | | | | | AGCGAACGC | |
| | | | | | | | AGACGCGGT | |
| | | | | | | | TCGCGTCGT | |
| | | | | | | | CCGCCTCAG | |
| | | | | | | | TTTCGGCCCT | |
| | | | | | | | TGTTTTGACT | |
| | | | | | | | CGGGAGGCG | |
| | | | | | | | TGTCCAAAC | |
| | | | | | | | GCCAGGTAA | |
| | | | | | | | CCACTATAT | |
| | | | | | | | AGTGAGCAC | |
| | | | | | | | GGTAGCATT | |
| | | | | | | | AGTCGGCAG | |
| | | | | | | | GAGAAGCAC | |
| | | | | | | | TTTAGCAGC | |
| | | | | | | | ATCTAGAAC | |
| | | | | | | | AGCAGCCTG | |
| | | | | | | | TAAGTACAT | |
| | | | | | | | TTAAGATTT | |
| | | | | | | | GTTTCAGTT | |
| | | | | | | | GTTGTGTAT | |
| | | | | | | | GGTTTGAGG | |
| | | | | | | | ACTTGTTTCC | |
| | | | | | | | AGCTGTTTG | |
| | | | | | | | TGTAAAGTT | |
| | | | | | | | GTAGGACAT | |
| | | | | | | | TTAAACTTG | |
| | | | | | | | CTTTCAGTTG | |
| | | | | | | | TGTATAATA | |
| | | | | | | | CTAGAAGTT | |
| | | | | | | | GTTTAGCCA | |
| | | | | | | | CTGTTTCCTT | |
| | | | | | | | GGTTACTAT | |
| | | | | | | | AAGAGCTTG | |
| | | | | | | | TGTAGCGAA | |
| | | | | | | | CGCAGACGC | |
| | | | | | | | GGTTCGCGT | |
| | | | | | | | CGTCCGCCT | |
| | | | | | | | CAGTTTCGG | |
| | | | | | | | CCCTTGTTTT | |
| | | | | | | | GACTCTCGA | |
| | | | | | | | GGCGTGTCC | |
| | | | | | | | AGCTGAATT | |
| | | | | | | | CAATCAGCT | |
| | | | | | | | AGTGCTTTG | |
| | | | | | | | GGGTTATAT | |
| | | | | | | | AAACAACTA | |
| | | | | | | | GTTCACCGC | |
| | | | | | | | GGCAGCGGT | |
| | | | | | | | CGCGGCAGC | |
| | | | | | | | CTCGTGTGA | |
| | | | | | | | AGACCGACG | |
| | | | | | | | AGGGTAAAG | |
| | | | | | | | ACCATCGAC | |
| | | | | | | | TCTACCTGC | |
| | | | | | | | GCGACTCCA | |
| | | | | | | | CCGAGCAAA | |
| | | | | | | | GACACCGAC | |
| | | | | | | | AAAGCACTT | |
| | | | | | | | GAGTACTTT | |
| | | | | | | | ACTGTATTG | |
| | | | | | | | TTTTACTTTA | |
| | | | | | | | CACTTATTTT | |
| | | | | | | | TTGTTGTCA | |
| | | | | | | | GTGCACTTTT | |
| | | | | | | | ATT | |
| L2-2_Dre | Danio | RT and | TGAG | (SEQ ID NO: 401) | (SEQ ID | a | (SEQ ID NO: | (SEQ ID |
| (2) | rerio | EN | CACG | MCFLIPVVTNTRKTREVRCRRN | NO: 500) | | 701) | NO: 801) |
| | | | GTAG | PHNLRSIHVSTISQLSLSVGLWN | GTTTCGC | | GCAGGAGAA | TAATCT |
| | | | CATTA | CQSAVNKADFITSIATYSDYNL | GTCGTCC | | GCACTTTAG | GCAATT |
| | | | GTCGa | MALTETWLRPEDTATHATLSA | GCCTCA | | CAGCATCTA | GCCTCT |
| | | | (SEQ | NFSFSHTPRQTGRGGGTGLLISK | GTTTCGG | | GAACAGCAG | CTGAAT |
| | | | ID NO: | EWKFTLIPSLPTISSFEFHAVTIIH | CCCTTGT | | CCTGTAAGT | ATCACA |
| | | | 37657) | PFYINVVVIYRPPGKLGHFLDEL | TTTGACT | | ACATTTAAG | CTAACT |
| | | | | DVLLSSFSNFATPLLVLGDFNIY | CGGGAG | | ATTTGTTTCA | GTACCC |
| | | | | VDKPQAADFQTLLASFDLKRAP | GCGTGTC | | GTTGTTGTGT | AAAAA |
| | | | | TSATHKSGNQLDLIYTRHCFTD | CAAACG | | ATGGTTTGA | AAAAA |
| | | | | QTIVTPLQISDHFLLSLNIHITPEP | CCAGGT | | GGACTTGTT | AAAAA |
| | | | | PHTPTLVTFRRNLRSLSPNRLSTI | AACCAC | | TCCAGCTGT | AAAAA |
| | | | | VSDSLPPSRKLTALDSNSATNTL | TATATAG | | TTGTGTAAA | AAAAA |
| | | | | CSTLASCLDRLCPLASRPARASP | TGAGCA | | GTTGTAGGA | ATAAAA |
| | | | | PAPWLSDALREHRSKLRAAERI | CGGTAG | | CATTTAAAC | AWACT |
| | | | | WRKTKNPAHLLTYQTLLSSFSA | CATTAGT | | TTGCTTTCAG | ACTAAT |
| | | | | EVTSAKQTYYRLKINNATNPRL | CG | | TTGTGTATA | ACTTCC |
| | | | | LFKTFSSLLYPPPPPASSTLTTDD | | | ATACTAGAA | CTTCTT |
| | | | | FATFFCTKTAKISAQFAAPTTNT | | | GTTGTTTAG | AGACTT |
| | | | | LVLSSHATTCPLDPIPSHLLQAIS | | | CTTGGTTACT | CCTGAA |
| | | | | PAVIPTLTHIINTSLDSGLFPTTF | | | ATAAGAGCT | ACTTGC |
| | | | | KQARVTPLLKKPNLDHTLLENY | | | TGTGTAGCG | CTATAG |
| | | | | RPVSLLPFMAKILEKVVFNQVL | | | AACGCAGAC | CACTTA |
| | | | | DFLTQNNLMDNKQSGFKKGHS | | | GCGGTTCGC | TTCATT |
| | | | | TETALLSVVEDLRLAKADSKSS | | | GTCGTCCGC | GTTGCT |
| | | | | VLILLDLSAAFDTVNHQILLSTL | | | CTCAGTTTC | CTTAGT |
| | | | | ESLGVAGTVIQWFRSYLSDRSF | | | GGCCCTTGT | TGTGTA |
| | | | | RVSWRGEVSNLQHLNTGVPQG | | | TTTGACTCG | AATTGC |
| | | | | SVLGPLLFSIYTSSLGPVIQRHGF | | | GGAGGCGTG | TTCCTT |
| | | | | SYHCYADDTQLYLSFHPDDPSV | | | TCCAAACGC | GTCCTC |
| | | | | PARISACLLDISHWMKDHHLQL | | | CAGGTAACC | ATTTGT |
| | | | | NLAKTEMLVVSANPTLHHNFSI | | | ACTATATAG | AAGTCG |
| | | | | QMDGATITASKMVKSLGVTIDD | | | TGAGCACGG | CTTTGG |
| | | | | QLNFSDHISRTARSCRFALYNIR | | | TAGCATTAG | ATAAAA |
| | | | | KIRPFLSEHAAQLLVQALVLSK | | | TCGGCAGGA | GCGTCT |
| | | | | LDYCNSLLAGLPANSIKPLQLL | | | GAAGCACTT | GCTAAA |
| | | | | QNAAARVVFNEPKRAHVTPLL | | | TAGCAGCAT | TGACTA |
| | | | | VRLHWLPVAARIKFKALMFAY | | | CTAGAACAG | AATGTA |
| | | | | KVTSGLAPSYLLSLLQIYVPSRN | | | CAGCCTGTA | AATGTA |
| | | | | LRSVNERRLVVPSQRGKKSLSR | | | AGTACATTT | AATGT |
| | | | | TLTLNLPSWWNELPNCIRTAES | | | AAGATTTGT | |
| | | | | LAIFKKRLKTQLFSLHFTS | | | TTCAGTTGTT | |
| | | | | | | | GTGTATGGT | |
| | | | | | | | TTGAGGACT | |
| | | | | | | | TGTTTCCAG | |
| | | | | | | | CTGTTTGTGT | |
| | | | | | | | AAAGTTGTA | |
| | | | | | | | GGACATTTA | |
| | | | | | | | AACTTGCTTT | |
| | | | | | | | CAGTTGTGT | |
| | | | | | | | ATAATACTA | |
| | | | | | | | GAAGTTGTT | |
| | | | | | | | TAGCCACTG | |
| | | | | | | | TTTCCTTGGT | |
| | | | | | | | TACTATAAG | |
| | | | | | | | AGCTTGTGT | |
| | | | | | | | AGCGAACGC | |
| | | | | | | | AGACGCGGT | |
| | | | | | | | TCGCGTCGT | |
| | | | | | | | CCGCCTCAG | |
| | | | | | | | TTTCGGCCCT | |
| | | | | | | | TGTTTTGACT | |
| | | | | | | | CGGGAGGCG | |
| | | | | | | | TGTCCAAAC | |
| | | | | | | | GCCAGGTAA | |
| | | | | | | | CCACTATAT | |
| | | | | | | | AGTGAGCAC | |
| | | | | | | | GGTAGCATT | |
| | | | | | | | AGTCGGCAG | |
| | | | | | | | GAGAAGCAC | |
| | | | | | | | TTTAGCAGC | |
| | | | | | | | ATCTAGAAC | |
| | | | | | | | AGCAGCCTG | |
| | | | | | | | TAAGTACAT | |
| | | | | | | | TTAAGATTT | |
| | | | | | | | GTTTCAGTT | |
| | | | | | | | GTTGTGTAT | |
| | | | | | | | GGTTTGAGG | |
| | | | | | | | ACTTGTTTCC | |
| | | | | | | | AGCTGTTTG | |
| | | | | | | | TGTAAAGTT | |
| | | | | | | | GTAGGACAT | |
| | | | | | | | TTAAACTTG | |
| | | | | | | | CTTTCAGTTG | |
| | | | | | | | TGTATAATA | |
| | | | | | | | CTAGAAGTT | |
| | | | | | | | GTTTAGCCA | |
| | | | | | | | CTGTTTCCTT | |
| | | | | | | | GGTTACTAT | |
| | | | | | | | AAGAGCTTG | |
| | | | | | | | TGTAGCGAA | |
| | | | | | | | CGCAGACGC | |
| | | | | | | | GGTTCGCGT | |
| | | | | | | | CGTCCGCCT | |
| | | | | | | | CAGTTTCGG | |
| | | | | | | | CCCTTGTTTT | |
| | | | | | | | GACTCTCGA | |
| | | | | | | | GGCGTGTCC | |
| | | | | | | | AGCTGAATT | |
| | | | | | | | CAATCAGCT | |
| | | | | | | | AGTGCTTTG | |
| | | | | | | | GGGTTATAT | |
| | | | | | | | AAACAACTA | |
| | | | | | | | GTTCACCGC | |
| | | | | | | | GGCAGCGGT | |
| | | | | | | | CGCGGCAGC | |
| | | | | | | | CTCGTGTGA | |
| | | | | | | | AGACCGACG | |
| | | | | | | | AGGGTAAAG | |
| | | | | | | | ACCATCGAC | |
| | | | | | | | TCTACCTGC | |
| | | | | | | | GCGACTCCA | |
| | | | | | | | CCGAGCAAA | |
| | | | | | | | GACACCGAC | |
| | | | | | | | AAAGCACTT | |
| | | | | | | | GAGTACTTT | |
| | | | | | | | ACTGTATTG | |
| | | | | | | | TTTTACTTTA | |
| | | | | | | | CACTTATTTT | |
| | | | | | | | TTGTTGTCA | |
| | | | | | | | GTGCACTTTT | |
| | | | | | | | ATT | |
| RTE- | Monodelphis | L1-EN, | NA | (SEQ ID NO: 402) | | | (SEQ ID NO: | (SEQ ID |
| 1_MD | domestica | RT, ZF | | MDSTAHPNQGRGLEKVSQTLP | | | 702) | NO: 802) |
| (1) | | | | ALQTPGQHTAAGGSSPLSGRNQ | | | gggtgtatggggtgc | tgaaactgca |
| | | | | RKNTKKLLLGAWNIRTLLDREN | | | tcagggaggggtag | caaagacaa |
| | | | | TPRPERRTALIGKELARYNIDIA | | | tatctctggtatggag | tagtcattctc |
| | | | | ALSETRLPEEGSLSEPTTGYTFF | | | ggcttgtcgtgccctc | gatcaccga |
| | | | | WKGRASNEDRIHGVGLAIKTSL | | | ctagggcagctctcc | gagactacc |
| | | | | LKQLPDLPVGISERLMKIRLPLS | | | agcctctgaccccca | act |
| | | | | KDRYATIISAYAPTLTSTEETIEQ | | | cctgacacccagctc | |
| | | | | FYSDLSAVLHSVPTNDKLILLG | | | tcacttgtggctccca | |
| | | | | DFNARVGQDHERWKGVLGKH | | | gtagctgctagcatgt | |
| | | | | GVGKMNNNGLLLLSKCSEFELT | | | ggcagcggccacac | |
| | | | | ITNTVFRMANKYKTTWMHPRS | | | cccgggcaacggct | |
| | | | | KQWHLIDYIIVRRRDIQDVKITR | | | tcgacaggccggct | |
| | | | | AMRGAECWTDHRLVRATLQM | | | aaaccttgtgagggt | |
| | | | | RIAPRHPKRAQTVRAFYNVSRL | | | agccatcgggtcatc | |
| | | | | RDPSYLQTFQSCLDDKLSAKGP | | | gacccctggtgaacc | |
| | | | | LTGSSTEKWNQFRDAVKETSK | | | agggctttgctcacc | |
| | | | | AVLGPKQRNHQDWFDENNTAI | | | cagcatgtgaagact | |
| | | | | EDLLSKKNKAFMEWQNNPNSA | | | gcttcggctgaacag | |
| | | | | PKKDRFKSLQATAQREIRKMQD | | | acggaagaaaccaa | |
| | | | | RWWEKKAEEIQRFADMKNYK | | | taagaaggttcaacg | |
| | | | | QFFSALKTVYGPLKPTTTPLLSS | | | gctgagagggcgac | |
| | | | | DGDTLIKDKKGISNRWKEHFSQ | | | gcagcaaagcactgt | |
| | | | | LLNRPSSVDQSALDQIPQNRTIE | | | ggagtgcttagggc | |
| | | | | QLDVPPSIEEVQKAIKQMSAGK | | | gtgttggagcacaaa | |
| | | | | APGKDGIPTEVYKALNGKALQ | | | ggacaacacggcca | |
| | | | | AFHIVLTSIWEEEDMPPELRDAS | | | tccaatgcagctgag | |
| | | | | IVALYKNKGSRAACDNYRGISL | | | gaagtctccagatgt | |
| | | | | LSTAGKILARVILNRLLSSVSEQ | | | aacaatttttcgtgcc | |
| | | | | NLPESQCGFRPDRSTIDMVFTV | | | actggacccaggctt | |
| | | | | RQMQEKCLEQNLSLYIVFIDLT | | | ccaacgccgagaga | |
| | | | | KAFDTVNRDALWVILSKLGCPA | | | gtgggactgtctctgt | |
| | | | | KFVKLIQLFHVDMTGEVLSGGE | | | gcatcggcttttccac | |
| | | | | TSDRFNISNGVKQGCVLAPVLF | | | ttaaatctctttcacgc | |
| | | | | NLFFTQVLRHAVMDLDLGVYI | | | acaagtatctttgtgc | |
| | | | | KYRLDGSLFDLRRLTAKTKTTE | | | acactcatctatccta | |
| | | | | RLILEALFADDCALMAHQENHL | | | accccgtccaccctc | |
| | | | | QTIVDRFSTATKLFGLTISLSKTE | | | ttcaagacctgcggc | |
| | | | | VLFQPAPGRPTNQPCITIDGTQL | | | gatgggggagtggc | |
| | | | | SNVNTFKYLGSTIANDGSLDHEI | | | gacgcaacaggtgg | |
| | | | | NARIQKASQALGRLRCKVLQH | | | aggtgaccactggc | |
| | | | | RGVSTATKLKVYNAVVLSSLLY | | | agttgtagtcacgatc | |
| | | | | GCETWTLYRKHMKQLEQFHQR | | | ctgcacgtaggcgg | |
| | | | | SLRSIMRIRWQDRITNQEVLDR | | | cccacggaccagtg | |
| | | | | ANSTSIEVMVLKTQLRWSGHVI | | | gtcgctcggccctgt | |
| | | | | RMDPQRIPRQVFYGELSAGLRK | | | gggcagcagggac | |
| | | | | QGRPKKRFKDQLKSNLKWAGI | | | gttcggcagcatcct | |
| | | | | TPKQLELAASDRSSWRTHINHA | | | gggcgactgagcag | |
| | | | | ATTFEDERRRRLAAARERRHQA | | | ccctctctag | |
| | | | | TTAPPVTTGVPCPMCHKLCASA | | | | |
| | | | | FGLQSHMRVHRR | | | | |
| RTE- | Monodelphis | L1-EN, | NA | (SEQ ID NO: 403) | | | (SEQ ID NO: | (SEQ ID |
| 1_MD | domestica | RT, ZF | | MDSTAHPNQGRGLEKVSQTLP | | | 703) | NO: 803) |
| (2) | | | | ALQTPGQHTAAGGSSPLSGRNQ | | | gggtgtatggggtgc | tgaaactgca |
| | | | | RKNTKKLLLGAWNIRTLLDREN | | | tcagggaggggtag | caaagacaa |
| | | | | TPRPERRTALIGKELARYNIDIA | | | tatctctggtatggag | tagtcattctc |
| | | | | ALSETRLPEEGSLSEPTTGYTFF | | | ggcttgtcgtgccctc | gatcaccga |
| | | | | WKGRASNEDRIHGVGLAIKTSL | | | ctagggcagctctcc | gagactacc |
| | | | | LKQLPDLPVGISERLMKIRLPLS | | | agcctctgaccccca | act |
| | | | | KDRYATIISAYAPTLTSTEETIEQ | | | cctgacacccagctc | |
| | | | | FYSDLSAVLHSVPTNDKLILLG | | | tcacttgtggctccca | |
| | | | | DFNARVGQDHERWKGVLGKH | | | gtagctgctagcatgt | |
| | | | | GVGKMNNNGLLLLSKCSEFELT | | | ggcagcggccacac | |
| | | | | ITNTVFRMANKYKTTWMHPRS | | | cccgggcaacggct | |
| | | | | KQWHLIDYIIVRRRDIQDVKITR | | | tcgacaggccggct | |
| | | | | AMRGAECWTDHRLVRATLQM | | | aaaccttgtgagggt | |
| | | | | RIAPRHPKRAQTVRAFYNVSRL | | | agccatcgggtcatc | |
| | | | | RDPSYLQTFQSCLDNKLSAKGP | | | gacccctggtgaacc | |
| | | | | LTGSSTEKWNQFRDAVKETSK | | | agggctttgctcacc | |
| | | | | AVLGPKQRNHQDWFDENNTAI | | | cagcatgtgaagact | |
| | | | | EDLLSKKNKAFMEWQNNPNSA | | | gcttcggctgaacag | |
| | | | | PKKDRFKSLQATAQREIRKMQD | | | acggaagaaaccaa | |
| | | | | RWWEKKAEEIQRFADMKNYK | | | taagaaggttcaacg | |
| | | | | QFFSALKTVYGPLKPTTTPLLSS | | | gctgagagggcgac | |
| | | | | DGDTLIKDKKGISNRWKEHFSQ | | | gcagcaaagcactgt | |
| | | | | LLNRPSSVDQSALDQIPQNRSIE | | | ggagtgcttagggc | |
| | | | | QLDVPPSIEEVQKAIKQMSAGK | | | gtgttggagcacaaa | |
| | | | | APGKDGIPTEVYKALNGKALQ | | | ggacaacacggcca | |
| | | | | AFHIVLTSIWEEEDMPPELRDAS | | | tccaatgcagctgag | |
| | | | | IVALYKNKGSRAACDNYRGISL | | | gaagtctccagatgt | |
| | | | | LSTAGKILARVILNRLLSSVSEQ | | | aacaatttttcgtgcc | |
| | | | | NLPESQCGFRPDRSTIDMVFTV | | | actggacccaggctt | |
| | | | | RQMQEKCLEQNLSLYIVFIDLT | | | ccaacgccgagaga | |
| | | | | KAFDTVNRDALWVILSKLGCPA | | | gtgggactgtctctgt | |
| | | | | KFVKLIQLFHVDMTGEVLSGGE | | | gcatcggcttttccac | |
| | | | | TSDRFNISNGVKQGCVLAPVLF | | | ttaaatctctttcacgc | |
| | | | | NLFFTQVLRHAVMDLDLGVYI | | | acaagtatctttgtgc | |
| | | | | KYRLDGSLFDLRRLTAKTKTTE | | | acactcatctatccta | |
| | | | | RLILEALFADDCALMAHQENHL | | | accccgtccaccctc | |
| | | | | QTIVDRFSTATKLFGLTISLSKTE | | | ttcaagacctgcggc | |
| | | | | VLFQPAPGRPTNQPCITIDGTQL | | | gatgggggagtggc | |
| | | | | SNVNTFKYLGSTIANDGSLDHEI | | | gacgcaacaggtgg | |
| | | | | NARIQKASQALGRLRCKVLQH | | | aggtgaccactggc | |
| | | | | RGVSTATKLKVYNAVVLSSLLY | | | agttgtagtcacgatc | |
| | | | | GCETWTLYRKHMKQLEQFHQR | | | ctgcacgtaggcgg | |
| | | | | SLRSIMRIRWQDRITNQEVLDR | | | cccacggaccagtg | |
| | | | | ANSTSIEVMVLKTQLRWSGHVI | | | gtcgctcggccctgt | |
| | | | | RMDPQRIPRQVFYGELSAGLRK | | | gggcagcagggac | |
| | | | | QGRPKKRFKDQLKSNLKWAGI | | | gttcggcagcatcct | |
| | | | | TPKQLELAASDRSSWRTHINHA | | | gggcgactgagcag | |
| | | | | ATTFEDERRRRLAAARERRHQA | | | ccctctctag | |
| | | | | TTAPPVTTGVPCPMCHKLCASA | | | | |
| | | | | FGLQSHMRVHRR | | | | |
| Vingi- | Anolis | DNAse- | NA | (SEQ ID NO: 404) | | | (SEQ ID NO: | (SEQ ID |
| 1_Acar | carolinensis | like, RT | | MDEYQRSLSRPLLTIMSINIEGL | | | 704) | NO: 805) |
| (1) | | | | SLAKEELLAKMSEDISCDILCIQ | | | GGGGGACAC | TAGTTG |
| | | | | ETHRDITMRRPKILGMQLAVER | | | GGAAAGAGC | CTTGTG |
| | | | | PHRQYGSAIFVRSGVAISATSLT | | | CTCCCCGAA | ATTTCT |
| | | | | EVNNIEILSVELDSCTVSSLYKP | | | GATTGAGTG | TTTCTT |
| | | | | PGADFYFTPPTSCHNHEAHFVV | | | AATTCAGTC | TTTTAT |
| | | | | GDFNSHSCVWGYDEDDRNGEA | | | GGGCGTCCC | TTTATT |
| | | | | VLTWADNSRMSLLHDSKLPPSF | | | CTGGGCAAC | TCCATT |
| | | | | NSGRWKRGYNPDLIFVKESISH | | | GTTTCTTGTA | ATTTGA |
| | | | | QCTKRVLNPIPNTQHRPICCVA | | | AGCGGCCGA | AATGTA |
| | | | | YAAVRPKSVPFRRRYNENKAN | | | TCTTTCCACC | TTTGCT |
| | | | | WTKFTETLEAAISDIEPSIENYD | | | CCAAAAGCA | GTACCA |
| | | | | LFVEAVKRSSRLSIPRGCRTSYL | | | TTGGATGA | ATGCTT |
| | | | | PGLNEESLNQLQEYLRLFQENP | | | | TTGACA |
| | | | | YSDGTIAAGQKLSTALANAKK | | | | CGAAAT |
| | | | | DRWIELLENLDMSKSSRKAWQ | | | | AAATAA |
| | | | | LLRRLDSDPLVNPGHANVTPDQ | | | | A |
| | | | | IAHQLIQNGKTNCSRIKMKINRV | | | | |
| | | | | PELETHQLSSPLNLKELREAIKR | | | | |
| | | | | CKTGKAPGLDDLMMEQIKHLG | | | | |
| | | | | PKAENWLLKFYNQCLAHKQIPR | | | | |
| | | | | AWRKTKIIAILKPGKDASNARN | | | | |
| | | | | YRPISLLCHLYKVYERMLLNRL | | | | |
| | | | | GPVIEPKLIAQQAGFRPGKNCT | | | | |
| | | | | GQILHLTEHIEEGYEKGCITGTV | | | | |
| | | | | FVDLTAAYDTVQHRKMLHKVY | | | | |
| | | | | HITRDFDFTKTVQTLLENRSFYV | | | | |
| | | | | EFQGQKSRWRRQKNGLPQGSV | | | | |
| | | | | LAPTLFNIFTNDQPQPPLTKSFIY | | | | |
| | | | | ADDLGLTTQAKDFETVEKQLT | | | | |
| | | | | NALKDLSSYYKENHLKPNPAKT | | | | |
| | | | | QVCAFHLRNREANRKLKVTWE | | | | |
| | | | | GQELEHCFHPKYLGVTLDRTLT | | | | |
| | | | | YRKHCMNTKHKVAARNNILRK | | | | |
| | | | | LTGSAWGADPQVIRTSALALSF | | | | |
| | | | | STAEYACPVWHKSAHAKQVDI | | | | |
| | | | | ALNETCRIITGCLKPTPVDKLYK | | | | |
| | | | | LAGIAPPDVRREVAANGERKKV | | | | |
| | | | | EHCESHPLHDYHPPPTRLKSRK | | | | |
| | | | | GFMRTTTPLDVPPATARVSLWA | | | | |
| | | | | AKPGNSNWMAPQEGLPPGANQ | | | | |
| | | | | EWATWKSLNRLRSGVGRSKDN | | | | |
| | | | | LARWHYLEESSTLCDCGAEQTT | | | | |
| | | | | QHMYACPQCPASCTEEELFKAT | | | | |
| | | | | DNAVAVARFWSKTI | | | | |
| Vingi- | Anolis | DNAse- | NA | (SEQ ID NO: 405) | | | (SEQ ID NO: | (SEQ ID |
| 1_Acar | carolinensis | like, RT | | MDEYQRSLSRPLLTIMSINIEGL | | | 705) | NO: 805) |
| (2) | | | | SLAKEELLAKMSEDISCDILCIQ | | | GGGGGACAC | TAGTTG |
| | | | | ETHRDITMRRPKILGMQLAVER | | | GGAAAGAGC | CTTGTG |
| | | | | PHRQYGSAIFVRSGVAISATSLT | | | CTCCCCGAA | ATTTCT |
| | | | | EVNNIEILSVELDSCTVSSLYKP | | | GATTGAGTG | TTTCTT |
| | | | | PGADFYFTPPTSCHNHEAHFVV | | | AATTCAGTC | TTTTAT |
| | | | | GDFNSHSCVWGYDEDDRNGEA | | | GGGCGTCCC | TTTATT |
| | | | | VLTWADNSRMSLLHDSKLPPSF | | | CTGGGCAAC | TCCATT |
| | | | | NSGRWKRGYNPDLIFVKESISH | | | GTTTCTTGTA | ATTTGA |
| | | | | QCTKRVLNPIPNTQHRPICCVA | | | AGCGGCCGA | AATGTA |
| | | | | YAAVRPKSVPFRRRYNFNKAN | | | TCTTTCCACC | TTTGCT |
| | | | | WTKFTETLEAAISDIEPSIENYD | | | CCAAAAGCA | GTACCA |
| | | | | LFVEAVKRSSRLSIPRGCRTSYL | | | TTGGATGA | ATGCTT |
| | | | | PGLNEESLNQLQEYLRLFQENP | | | | TTGACA |
| | | | | YSDGTIAAGQKLSTALANAKK | | | | CGAAAT |
| | | | | DRWIELLENLDMSKSSRKAWQ | | | | AAATAA |
| | | | | LLRRLDSDPLVNPGHANVTPDQ | | | | A |
| | | | | IAHQLIQNGKTNCSRIKMKINRV | | | | |
| | | | | PELETHQLSSPLNLKELREAIKR | | | | |
| | | | | CKTGKAPGLDDLMMEQIKHLG | | | | |
| | | | | AKAENWLLKFYNQCLAHKQIP | | | | |
| | | | | RAWRKTKIIAILKPGKDASNAR | | | | |
| | | | | NYRPISLLCHLYKVYERMLLNR | | | | |
| | | | | LGPVIEPKLIAQQAGFRPGKNCT | | | | |
| | | | | GQILHLTEHIEEGYEKGCITGTV | | | | |
| | | | | FVDLTAAYDTVQHRKMLHKVY | | | | |
| | | | | HITRDFDFTKTVQTLLENRSFYV | | | | |
| | | | | EFQGQKSRWRRQKNGLPQGSV | | | | |
| | | | | LAPTLFNIFTNDQPQPPLTKSFIY | | | | |
| | | | | ADDLGLTTQAKDFETVEKQLT | | | | |
| | | | | NALKDLSSYYKENHLKPNPAKT | | | | |
| | | | | QVCAFHLRNREANRKLKVTWE | | | | |
| | | | | GQELEHCFHPKYLGVTLDRTLT | | | | |
| | | | | YRKHCMNTKHKVAARNNILRK | | | | |
| | | | | LTGSAWGADPQVIRTSALALSF | | | | |
| | | | | STAEYACPVWHKSAHAKQVDI | | | | |
| | | | | ALNETCRIITGCLKPTPVDKLYK | | | | |
| | | | | LAGIAPPDVRREVAANGERKKV | | | | |
| | | | | EHCESHPLHGYHPPPTRLKSRK | | | | |
| | | | | GFMRTTTPLDVPPAAARVSLW | | | | |
| | | | | AAKPGNSNWMAPQEGLPPGAN | | | | |
| | | | | QEWATWKSLNRLRSGVGRSKD | | | | |
| | | | | NLARWHYLEESSTLCDCGAEQ | | | | |
| | | | | TTQHMYACPQCPASCTEEELFK | | | | |
| | | | | ATDNAVAVARFWSKTI | | | | |
| CR1-1_PH | Parhyale | DNAse I- | NA | (SEQ ID NO: 406) | | | (SEQ ID NO: | (SEQ ID |
| | hawaiensis | like, RT | | MLYTIFL VYGILCFFFVYFCIYL | | | 706) | NO: 806) |
| | | | | YITVYLCFLFFLFLLLCGDVESN | | | gcgtggcctgcgcgt | tagcagcgtt |
| | | | | PGPGRARGCRLLYCNIRGLHAN | | | tatcaggccgccccc | ttgggcttcc |
| | | | | LAELDFVSRGVDVVCCSETLVA | | | attgtccggccaccg | cggtgttgca |
| | | | | GRRHDAELALAGFQSPFRRLCG | | | gacgcctctttgtttgt | agatgcttca |
| | | | | SGPGFRGMAVYVRSGFCAYRQ | | | atgagctggctttgg | tttgggttgta |
| | | | | SVHECACHEIIVVRVCGRLNNY | | | gtttggggctctgggt | ttacctgtcc |
| | | | | YLFSLYRSPATDDSLYDCLLTA | | | agcccctgagttgga | gtgggagct |
| | | | | MASIQSTDPKAAFVFVGDVNA | | | ggaaatgccactcta | caagcgtga |
| | | | | HHRDWLGSASPTDCHGVAALD | | | aaggcggatgagac | agttttaataa |
| | | | | FCTLSSCVQLVRGSTHIAGNCL | | | ctcttttggtccaacg | taataataa |
| | | | | DLVMTDVPDLMTVTVGSPIGSS | | | gcatccctgagagg | |
| | | | | DHSHLVVSLDLNQVVPVVDTR | | | agatgaacaggctg | |
| | | | | RVVFVKSRANWVAITRAVRTLP | | | gtgccatagctgggt | |
| | | | | WRQIIHSEDPVSELNDLTVSILE | | | actcagtcttccgca | |
| | | | | RFVPKRTILVRSRDKPWFDDQC | | | gtctgctcctctctctc | |
| | | | | RLAFEAKQAAYRAWRRSRDRT | | | ttccatcaggtagtac | |
| | | | | LWQTYVDRRAEAKRVYEEAQR | | | cacctgatgctcttttc | |
| | | | | RLRQRSRESLLSIDHPHRWWSE | | | ctttcctttctcttctt | |
| | | | | LKGSVFGAEPSLPPLVGPGGGII | | | cttcttttcggccaaag | |
| | | | | TDPLARAELLSAHFDGKQSRDV | | | cctgtactggtgatgg | |
| | | | | IALPHGCHPEPRLTSLAFRSGAI | | | cagggtctggggcg | |
| | | | | KVLLEGLDPYGGVDPVGMFPLF | | | cgtagatagctggcc | |
| | | | | YKQLADVLAPKLAVIFRRLIRL | | | gctttcgtagctatcg | |
| | | | | GNFPRCWRTGNITPIPKGPVSPY | | | ttactgtcgatttgttt | |
| | | | | VANYRPITLTPILSKVFEKLIAG | | | ttgctttatt | |
| | | | | KLGRFAEVTGLLPAGQFAYRIG | | | | |
| | | | | LGCCDALLSVSHHLQSALDCRS | | | | |
| | | | | EARLVQLDFSAAFDRVNHRGLL | | | | |
| | | | | YKLESFGVGGRVLSIIRDFVSER | | | | |
| | | | | TQSVSVDGVLSASVGVVSGVPQ | | | | |
| | | | | GSVLGPLLFVLYTSDMFSSLEN | | | | |
| | | | | TLINYADDSTLMAVIPAPRLRD | | | | |
| | | | | AVAQSLNRDLSRISAWCSAWS | | | | |
| | | | | MKLNASKTKSMIISRSRTLVPQ | | | | |
| | | | | HPQLEIDDTLLQESSSLEILGVV | | | | |
| | | | | FDEKLTFEPHIRRLVSRASTKIG | | | | |
| | | | | LLRKVNSVFGDSQVARRCFYAF | | | | |
| | | | | LLPVLEYCSPVWASAADTHLRL | | | | |
| | | | | LDRLVSSASRLCADNDLVNLSH | | | | |
| | | | | RRRVAELCMFYKVYNNERHWL | | | | |
| | | | | YSSLPALKVFGRETRAACGAHS | | | | |
| | | | | FTLEAVRCRTNQFCRCFVPWSA | | | | |
| | | | | KVWNLLPASAFGRIGLQAFKSA | | | | |
| | | | | VNGFLLDFL | | | | |
| Crack- | Branchiostoma | RT and | NNNN | (SEQ ID NO: 407) | | | (SEQ ID NO: | (SEQ ID |
| 28_BF | floridae | EN | NNNN | MWEKATSNVHLQSGTWITQNA | | | 707) | NO: 807) |
| | | | NNNN | YSVKINPGLSGVRLIGRSTTCTE | | | AGCCCTAGT | AACTTG |
| | | | NNNN | RTERTLNLLVCATLLLAGDVSP | | | CCCT | ATCTGG |
| | | | NNNT | NPGPDTGGLPVWRKGIVYAFY | | | | CTGATG |
| | | | NNNN | NVVSLPRHLDEIQQLLLRNTRIH | | | | CTCCGT |
| | | | NCNN | VLGLNETRLSDSIPDSSVDINGY | | | | AGTCTG |
| | | | GTNN | TLYRTDRDRQGGGVGVYVKQT | | | | GACTTT |
| | | | NNAN | IASQRRCELEQEDLEVCCVEIKP | | | | GATATT |
| | | | NNNN | EKARKTLLTCVYRPPTSGPDWR | | | | GGATAA |
| | | | NNNN | NSAESLVHKLNQTAEKENADV | | | | TTTTAT |
| | | | NNNN | AIMGDFNSDLLTSTQAMSSVEF | | | | TATGTA |
| | | | NCNN | LMGLYQLVPVIREPTRITEKTES | | | | TTGTAT |
| | | | NNNN | CIDNIFVSNPDRYKSSASVAWG | | | | ATGTGC |
| | | | NNNN | PSDHNLILTCAKAGSEAGAAHR | | | | TATGTG |
| | | | NNNN | CEYRSYKLYTQQSFIDSLKSVR | | | | TACTTT |
| | | | NNTN | WDTVFDCTDVSEAWNAFKDIF | | | | GTACTT |
| | | | NNNN | LNVADEHAPLRTKTARENNRP | | | | TTATGT |
| | | | NNNN | APWMTDTVKNMMGRRDAARR | | | | CCAGGA |
| | | | NNNN | KAIRTKDVQDWDTYRSLRNQT | | | | TTACCT |
| | | | NNNN | TSIIRKEKKSHFATAVSEAKGDQ | | | | GAAAA |
| | | | NNNT | SLMWKIINSFTGKSKSTKRVQK | | | | GCAGGC |
| | | | NNNN | LLRADNTSMSDPGEMAQEFND | | | | CGATTT |
| | | | NNNN | YFTSCASRLTDGMPDSEEDPLR | | | | CCGGCC |
| | | | NNNNn | HIPDSTTKFSFDCVEETEVLNEL | | | | TGAGAT |
| | | | ntnnnnn | QKLKTKKATGLDKIPAKLLKDS | | | | GTAATT |
| | | | nnnnnn | APVVAKPLAHIFNLSLASGEVPS | | | | ACTGGT |
| | | | nnnnnn | DWKEAQITPVHKSGSCADVGN | | | | AAAATA |
| | | | nnnnn | YRPVSVLSVTSKVMEKLVCNQ | | | | AATAAA |
| | | | nnnnnn | VTRYLTRCKLLTTHQSGFRRHH | | | | GTGAAG |
| | | | nnnnnn | STATAVQKVVEDITSGYNCSKV | | | | TGAA |
| | | | nnnngc | TVALFLDLRKAFDSVNHEIMLS | | | | |
| | | | nnnnnn | KLKKFGFDSDAMKWFTSYLSE | | | | |
| | | | nntnnnn | RLQCTCLQGQYSSKTRVSCGVP | | | | |
| | | | nnnnnn | QGSVLGPLLFCLYVNDLPNVIQ | | | | |
| | | | nnnnnn | KCSIHMYADDTVLYYSAVSVK | | | | |
| | | | nnnnnn | VCEETVSMDMKRVVKWLSENR | | | | |
| | | | nnntnnn | LLLHPDKTKSMLFGLPQKLKHA | | | | |
| | | | nnnnnn | GTTVNITDGVNVYEQVDSFTYL | | | | |
| | | | nnannn | GITLDPALRWAAHVQKITKKLL | | | | |
| | | | nnnnnn | SGLGAMGRARAFVTNEVLKTM | | | | |
| | | | (SEQ | YQTLLLAHLEYCATAWLPSLA | | | | |
| | | | ID NO: | QGNKTLMLQLDRLVNRAARLI | | | | |
| | | | 37658) | TGHKLRDHVTVDNLRAEAGIDS | | | | |
| | | | | VRKRTEITTLVTVFKTIRGKAPA | | | | |
| | | | | YLASLFKWEAPPTMSVRPTRSE | | | | |
| | | | | VKRLRDYDPHLLWCPPARVIAF | | | | |
| | | | | RNSLQSYGPFLWNSLPLKQRQL | | | | |
| | | | | LSLRTFKKFIEN | | | | |
| L2-5_GA | Gasterosteus | RT and | NNNN | (SEQ ID NO: 408) | | | (SEQ ID NO: | (SEQ ID |
| | aculeatus | EN, signal | NNNN | MRRLLLLFLIMCLTPSPVPVRIS | | | 708) | NO: 808) |
| | | peptide | NNNN | SRRYYRPRARSALYRNLSSLSY | | | CAGTGTGCA | TAAAGA |
| | | | NNNN | PTRSTHVQHLVTGGLWNCQSA | | | TCTCTTCTAC | CTAACA |
| | | | NNNN | TRKADFISGFAIQQSLDFLALTE | | | AAGGCCACC | AATTGT |
| | | | NNNN | TWITPENTSTPAALSSAFSFSHT | | | AGACATCCA | AGCACT |
| | | | NNNN | PRPTGRGGGTGLLISPKWSFSLY | | | GGCGACCTG | TAAATT |
| | | | NNNN | PLPPSTPLSFEFHAVTITHPVQLT | | | AAATCGGAC | GTACTT |
| | | | NNNN | IIVLYRPPGSLGHFLEELDILLSN | | | TAAGTATCT | GTAACG |
| | | | NNNN | FPENGPPLILLGDFNIQTEKSSDL | | | CTTTCTTTAA | TCACTC |
| | | | NNNN | LHLLSSFALSLSPSPPTHKAGNH | | | ACCAAGCTA | ATCTAT |
| | | | NNNN | LDYIFTRNCSTTNLSVTPLHVSD | | | GTCTGCTAA | AGCAAA |
| | | | NNNN | HFFISYSLPLSITNKPPSLTNSIPA | | | ATTTGCTGTT | TTGTAA |
| | | | NNNN | RRNIRSLSPSSLASSVLSALPSTD | | | GATTCTAGT | ATTGGC |
| | | | NNNN | SFSLLHPNAAAETLLSTLSSSLD | | | GTCTTAAGT | TTATTT |
| | | | NNNN | SLCPLTTRRTGKSPPAPWLSQPV | | | TAATTTGATT | GAGGA |
| | | | NNNN | RAMRATMRASERRWRKYKRP | | | GCTGATTTG | AATTGC |
| | | | NNNN | DDLLEFQSLLSSFSASISAAKSSF | | | CTTTAGTTTT | ACTTTC |
| | | | NNNN | YQSKIESSFSNPKKLFSIFSNLLE | | | TGTCGTCTC | TTGTTT |
| | | | NNNN | PPTPPPPSTLLPGDFVNYFTKKI | | | ACGGCCAGT | CTTGTT |
| | | | NNNN | ADIRSSFSNPPPTSRVPPTSPLSP | | | TCTCTTTGTT | CTCCTG |
| | | | NNNN | SLSSFTALSPNQILTLVTSARPTT | | | TGAAGTTAT | AGTTTG |
| | | | NNNN | CPLDPIPSHLLQSIAPDLLPFLTC | | | CTTGGTTGCT | TACCCT |
| | | | NNNN | LINNALSSGCFPNSLKEARVNPL | | | AGTTTGCTC | ATGGTT |
| | | | NNNNn | LKKPTLNPSEENNYRPVSLLPFL | | | AAGCTCTTT | GAATGC |
| | | | nnnnnn | SKTLERAIFNQLSSYLHCNNLLD | | | CAGTCTTTTA | ACTTAT |
| | | | nnnnnn | PHQSGFKAGHSTETALLAVSEQ | | | GTGCCAGTT | TGTACG |
| | | | nnnnnn | LHTARAASLSSVLILLDLSAAFD | | | TGTGTTCTA | TCGCTT |
| | | | nnnnnn | TVNHQILISSLQELGVTGSALSL | | | GATTCTACT | TGGATA |
| | | | nnnnnn | LSSYLDGRTYRVTWRGSVSEPC | | | ATTAAGTTA | AAAGCG |
| | | | nnnnnn | PLTTGVPQGSVLGPLLFSLYTNS | | | ACTGCCAGT | TCAGCT |
| | | | nnnnnn | LGAVIRSHGFSYHSYADDTQLIL | | | GTGCCCTCC | AAATGA |
| | | | nnnnnn | SFPHSDTQVAARISACLTDISQW | | | TAACTCTGC | CATGTA |
| | | | nnnnnn | MSAHHLKINPDKTELLLFPGKD | | | TAGGTTTTG | ATGTAA |
| | | | nnnnnn | SLTQDLTVNFGNSVLTPTSTAK | | | ACCTGATCT | TGTAAT |
| | | | nnnnnn | NLGVTLDSQLSLTPNITATTRSC | | | AAGTCTGTT | GTAATG |
| | | | nnnnnn | RYTLYNIRRIRPLLTQKAAQVLI | | | TTGCCTTGTT | |
| | | | nnnnnn | QALVISRLDYCNSLLAGLPATAI | | | TTCTGAAGC | |
| | | | nnnnnn | RPLQLIQNAAARLVFNLPKFSH | | | AAATAAGAC | |
| | | | nnnnnn | TTPLLRSLHWLPVAARIQFKTL | | | TTGTATCCTT | |
| | | | nnnnnn | VLTYHAVNGSGPAYIQDMVKP | | | AAACTCTCA | |
| | | | nnn | YIPTRTLRSASAKLLVPPSLRAK | | | TTTTGTCAA | |
| | | | | HSTRSRLFAVLAPKWWNELSE | | | AACACCACA | |
| | | | | DTRTAESLHIFRRKLKTHLFRLY | | | TGGTGTTTGT | |
| | | | | LD | | | TCACTGATT | |
| | | | | | | | AGGGTGTCA | |
| | | | | | | | AGCTATTGG | |
| | | | | | | | TGCTTTGCAT | |
| | | | | | | | TTTGAGGAG | |
| | | | | | | | TTTCTGTCGA | |
| | | | | | | | GGCCAATCG | |
| | | | | | | | ACCTTTTGTC | |
| | | | | | | | TCCTAAACG | |
| | | | | | | | ACGGGGGAG | |
| | | | | | | | CGGCCAGCG | |
| | | | | | | | CAGCCGCAG | |
| | | | | | | | CCGACTTCC | |
| | | | | | | | ACTAACGAG | |
| | | | | | | | GGAGTATTT | |
| | | | | | | | AACTAGAAA | |
| | | | | | | | TCGTGGGAA | |
| | | | | | | | GCGTTAGCT | |
| | | | | | | | TATCCTCGC | |
| | | | | | | | AGCACGAAG | |
| | | | | | | | ACGAGCAGA | |
| | | | | | | | ACAAAGACC | |
| | | | | | | | AGGGAGTCT | |
| | | | | | | | TTCGTGGAG | |
| | | | | | | | TCAAGACGA | |
| | | | | | | | GAGACAAAG | |
| | | | | | | | ACCAGGGAG | |
| | | | | | | | TCTTTCGTGG | |
| | | | | | | | AGTCAAGAC | |
| | | | | | | | GAGCAGAAC | |
| | | | | | | | AAAGACAAG | |
| | | | | | | | GGAGTCTTT | |
| | | | | | | | CGTGGCAGT | |
| | | | | | | | CTTCGGGGC | |
| | | | | | | | GGCTGA | |
| RTE-3_BF | Branchiostoma | RT and | NTNN | (SEQ ID NO: 409) | — | | (SEQ ID NO: | (SEQ ID |
| | floridae | EN | NNNN | MSGTPRVAFDSGKDLRNPIGQS | | | 709) | NO: 809) |
| | | | NNNN | PPALSRAAPGQLGTDPSRSACFI | | | TCTGTAAAT | TGACCT |
| | | | NTNN | GCLELRVLLDKWIVCRAPDKQE | | | GGCTGTGTG | GATACA |
| | | | NNNN | SKEKRRQKTQPIRIGSWNVRTM | | | ATGCGTCTA | GAGCGC |
| | | | NNNN | RTGLSDDLTVIEDIRKTAAIDRE | | | GTGTGTAGG | TACCAT |
| | | | NNNN | LYRLNIDIVALQETRLPDSGSLK | | | CGTAGCGGT | CATCTG |
| | | | NNNN | EDSYTFFWQGKGMEETREHGV | | | AGTGTGCTT | GAAAG |
| | | | NNNG | GFAVRNTLLHMIEPPTGGTERII | | | GCCACTTCT | ATGGAA |
| | | | NNNN | TLRLSTHEGPVNLLCVYAPTLQ | | | CGCTTTCAG | GGATGC |
| | | | NNNN | ATSEVKDQFYGQLDSAIKKIPVS | | | CCCTCACCG | CTACTA |
| | | | NNNN | EHIFILGDFNARVGTDQESWQT | | | CTGGCTAGC | C |
| | | | NNNN | VLGHHGIGKMNENGQRLLELC | | | GGAGCTGCA | |
| | | | NNNN | CYHNLCVTNTFFQNKAIHKAS | | | TGCAGCACT | |
| | | | NNNN | WRHPRSQRWHQLDLVITRRTSL | | | GCGAAAAGA | |
| | | | NNNN | NSVCNTRAYHSADCDTDHSLIA | | | GAGCCGAGT | |
| | | | NNNN | ARIKLRPKKLHHMKKKGQPKID | | | GCGTAAGTC | |
| | | | NNNN | VSKTMLPDRNQKFLECLEGTLN | | | TCTCCTGCC | |
| | | | TNNNT | NIQPQDAEHRWETLSKTIYSAA | | | AGTACATAG | |
| | | | NNNN | AQSYGKKERKNTDWFEAYISEL | | | CCTCTCCAC | |
| | | | NNNN | EPVMDTKRKALVSYKQNPSSQ | | | AGCAAGTCC | |
| | | | NNNN | NLQALKAARQEAQRASRRCAN | | | CCATTGCAG | |
| | | | ANNN | NYWLLLSERIQLASATGDIRRM | | | TGCCTCCTC | |
| | | | NNNN | YEGIKQATGKPIKKSAPLKAKS | | | GTGGCTACA | |
| | | | NNNnn | GEIITDKDKQMARWVEHYLDIY | | | GACGGAAAC | |
| | | | nnnnnn | STENSVSQDALDNIEDFSVLAEL | | | TGGGCACCG | |
| | | | nnnntnn | DADPTIEELSKAIDSMSNGKAP | | | TAAGGCCCC | |
| | | | nnnnnn | GEDNIPAEIIKSGKSVLLEPLHEL | | | AGGCTAAAC | |
| | | | nnnnnn | LRLCWKEGKVPQSMRNSKIVTL | | | TGCAGGGAG | |
| | | | gngnnn | YKNKGDRTDCNSYRGISLLSIV | | | CTCGGAGGA | |
| | | | nnntnnn | GKVFAKVVLTRLQVLADRVYP | | | GGTCTGGCC | |
| | | | nncnnn | ESQCGFRAERSTTDMIFSVRQL | | | CCCAGACGC | |
| | | | nnnnntn | QEKCREQQRPLYIAFIDLTKAFD | | | ACGGCATGC | |
| | | | nnnnnn | LVSRRGLFQLLRKIGCPPQLLDII | | | ATGGCCCAC | |
| | | | nnnnnn | ISFHEDMKGVVSFDGETSEPFAI | | | CGGCGTGTG | |
| | | | ntnnnnn | RSGVKQGCVLAPTLFGIFFSLLL | | | GACACGCCC | |
| | | | nnnnnn | KSAFGHSTQGVHLHTRSDGKLF | | | TGATGCCTG | |
| | | | nnannn | NLARLRAKTKVRSVLIRDMLFA | | | CGAACCAGA | |
| | | | nnnnnn | DDAALVAHVEDELQQLLNQFA | | | CCCCCAGCT | |
| | | | nnnnnn | HACSEFALTISIKKTVVMGQDV | | | ATGGGCAAA | |
| | | | nnnn | PQPPVVTIGSEVLEVTDHFTYLG | | | TAGCACGGG | |
| | | | (SEQ | STVTSNLSLDKEIDRRIARAAGV | | | TAGACGGAG | |
| | | | ID NO: | MTKLGTRVWNNSHLTLNTKLE | | | CTCGTCAGC | |
| | | | 37659) | VYRSCVLSTLLYGSETWTTYAK | | | CTTGGATGG | |
| | | | | QENRLESFHLRCLRRILGISWRD | | | CAGTTCGTC | |
| | | | | RVPNTTVLERSCSLSIHLLLCQR | | | TAGGGGAAG | |
| | | | | RLRWLGHVSRMKDGRIPKDILF | | | GAAAACCCT | |
| | | | | GELATGKRPVGRPALRFRDVCK | | | GATTCAAAA | |
| | | | | RDLKLTDIDPASWEQIAADRNR | | | ACCTCCGCT | |
| | | | | WRHTVKDGLAKGQERRTEHLE | | | GCCTTGCGG | |
| | | | | SRRRKRKEKPQQGNPSAFICPN | | | CTATACCCA | |
| | | | | CGRDCHARIGLQSHSRRCQPP | | | GTCCTGGGA | |
| | | | | | | | AAGGCTACG | |
| | | | | | | | GGAGTTAAC | |
| | | | | | | | CCAGAGAGA | |
| | | | | | | | AAATCCGGA | |
| | | | | | | | GTGGAGTAC | |
| | | | | | | | GTGAGGCGG | |
| | | | | | | | TTGGCTGTC | |
| | | | | | | | AAACTCTGT | |
| | | | | | | | CATCCTTCC | |
| | | | | | | | GGCAACTCC | |
| | | | | | | | TGCAGCCAA | |
| | | | | | | | ACCAACGCC | |
| | | | | | | | AAGTGTCAC | |
| | | | | | | | GCCTCGCGT | |
| | | | | | | | TCCCTTGGA | |
| | | | | | | | CCACGTCGG | |
| | | | | | | | TGAGGTCGA | |
| | | | | | | | GAGGGGGGT | |
| | | | | | | | CCTGTTGTGT | |
| | | | | | | | TTTTGGGCA | |
| | | | | | | | GCGCAGGTC | |
| | | | | | | | CTCCATAAA | |
| | | | | | | | CCTGCCCAG | |
| | | | | | | | GCTAGCGCT | |
| | | | | | | | CTGGAGAGG | |
| | | | | | | | CCACTCCAG | |
| | | | | | | | TCGCCCCCA | |
| | | | | | | | TCACTGGGG | |
| | | | | | | | GTGAGAAAC | |
| | | | | | | | AAACCGGGA | |
| | | | | | | | GACAGCAGT | |
| | | | | | | | TTACGGGTT | |
| | | | | | | | ATAAGTCCT | |
| | | | | | | | TGCTAAATT | |
| | | | | | | | GACGTAA | |
| RTE- | Locusta | RT and | NNNN | (SEQ ID NO: 410) | | | (SEQ ID NO: | (SEQ ID |
| 25_LMi | migratoria | EN | NNNN | MPRKNWNCGRRTGDEKRKMM | | | 710) | NO: 810) |
| | | | NNNN | FGCWNVQGISTKIDLLPAELDM | | | CCCGTGTGG | TAGTGT |
| | | | NNNN | FNIDVVVLSETKRKGKGEEELD | | | AGTTTGCTG | AAAACC |
| | | | NNNN | NYVHIWSGVSKAVRAKAGVSI | | | GTCTTCCATC | TTATGT |
| | | | NNAN | MIQKKWKKRITNWTFINERIITV | | | GGGCACCTC | ACTAGG |
| | | | NNNN | EMTLFAREVVIIGVYAPTNDTK | | | CCCAGGTGG | TGTATT |
| | | | NNNN | DKEKDAFWDTLRETIEKIPRRK | | | CGGATAGGG | CATTTC |
| | | | NNNN | ELIIMGDMNGRVGIRESCKIVG | | | GAATGCTCA | TGGGCG |
| | | | NNNA | KHGEAEYNDNGERLIDICAQFD | | | CCAGATATG | TATTAG |
| | | | NNNN | LKITNTFFKHKDIHKYTWQQNT | | | GTGGGTACC | TATGTT |
| | | | NCNN | KELRSIIDYIIIRQTSSFKAADVR | | | GGGGAAATA | GGAGGT |
| | | | NANN | SYRGAQCGSDHYLVKMKSFWP | | | AAATACCCG | AAACCT |
| | | | NNNN | WKNATNDTSNINKMNCSEKVQ | | | GGGTGGACC | CTGTAA |
| | | | NNNN | NVHFNIDSLQDESIRTFFKARME | | | AAAACCAGC | TGAGGA |
| | | | NNNN | RTLDESFEGSTEEIYEYIKTKVK | | | AACTGCTGC | CAATCT |
| | | | NNNN | NVASEVLGIKENNPKRAAEWW | | | CTTGTAGTA | CAATAA |
| | | | NNNN | SEEIETSVKEKRNAFVQWLNDK | | | TGATATTGG | TAAAAT |
| | | | NNNN | SEGTRSKYKEKKNEVEKKIRLA | | | CTTATCAAA | AAAATA |
| | | | NNNN | KNEAWERTCANVNSKLGFGRA | | | GGCTAAAGG | AA |
| | | | NNNN | KEAWSVLKALRQDTKGKSNLQ | | | AAGAAAACC | |
| | | | NNNN | LVTQKEWEEYFKKLLNEDRDE | | | TTGAATACA | |
| | | | NNNN | YLEEGTVEENEHHDDEILISESE | | | AATTACCTG | |
| | | | NNNN | VLQVLRTGKNGKSPGPGNINME | | | GTCCTCCAG | |
| | | | CNNNn | FLKYGGDKIVKLILQLFNKMLH | | | GTTGGGGGT | |
| | | | nnnnnn | GDSVPKEMKLGYISTIFKKGDR | | | TGTGCAGTG | |
| | | | nnnnan | KICSNYRGICVTNTLMRIFGKIIK | | | GGCCAGCTC | |
| | | | nnnnnn | NKLEKNFRTQQEQCGFTAGRSC | | | CTCACTCAC | |
| | | | nnnnnn | VDHIFTLRQILEKHREKSKNVG | | | ATAAAAATA | |
| | | | antnntn | LIFIDLEKAYDTVPRKLLWRAL | | | TAAAATGCT | |
| | | | nnnnnn | HRANINTSLIKIIEQMYKDNICQ | | | AAAAAACCT | |
| | | | nnnnnn | VKIGNTLSQKFRTSKGLLQGCP | | | AATAAT | |
| | | | nnnann | MSPTLFKIYIDICLRTWSQKCNS | | | | |
| | | | nnnnnn | MGLEIRDGVYLHHLLFADDQV | | | | |
| | | | nnnnng | VIAQDGEDANYMCNQLAIAYK | | | | |
| | | | nnnnnn | NWGLKINYQKTEYLTNDPHEL | | | | |
| | | | annnnnt | RIEGKKIKKVNTFCYLGSILETE | | | | |
| | | | nnnnnn | GKSDSEINKRISSGRKVIGMLNS | | | | |
| | | | annnnn | VLWSRNVMNRTKKIIYKSIFES | | | | |
| | | | nnnnnn | AVLYGAETWTINQKHTKKLQA | | | | |
| | | | annnnn | LEMDFWRRSARISRKEKRRNTE | | | | |
| | | | n (SEQ | VIKRMEIIERIDEVMDRKKLRW | | | | |
| | | | ID NO: | YGHVRRMEDTRIPKLVLEWQP | | | | |
| | | | 37660) | EGRRRRGRPVTTWIKNVQLTM | | | | |
| | | | | NRLGAEEEDTQDRHTWRNIVN | | | | |
| | | | | N | | | | |

### Retrotransposon Discovery Tools

As the result of repeated mobilization over time, transposable elements in genomic DNA often exist as tandem or interspersed repeats (Jurka Curr Opin Struct Biol 8, 333-337 (1998)). Tools capable of recognizing such repeats can be used to identify new elements from genomic DNA and for populating databases, e.g., Repbase (Jurka et al Cytogenet Genome Res 110, 462-467 (2005)). One such tool for identifying repeats that may comprise transposable elements is RepeatFinder (Volfovsky et al Genome Biol 2 (2001)), which analyzes the repetitive structure of genomic sequences. Repeats can further be collected and analyzed using additional tools, e.g., Censor (Kohany et al BMC Bioinformatics 7, 474 (2006)). The Censor package takes genomic repeats and annotates them using various BLAST approaches against known transposable elements. An all-frames translation can be used to generate the ORF(s) for comparison.

Other exemplary methods for identification of transposable elements include RepeatModeler2, which automates the discovery and annotation of transposable elements in genome sequences (Flynn et al bioRxiv (2019)). In addition to accomplishing this via available packages like Censor, one can perform an all-frames translation of a given genome or sequence and annotate with a protein domain tool like InterProScan, which tags the domains of a given amino acid sequence using the InterPro database (Mitchell et al. Nucleic Acids Res 47, D351-360 (2019)), allowing the identification of potential proteins comprising domains associated with known transposable elements.

Retrotransposons can be further classified according to the reverse transcriptase domain using a tool such as RTclass1 (Kapitonov et al Gene 448, 207-213 (2009)).

### Polypeptide Component of Retrotransposon Gene Modifying System

### RT Domain

In certain aspects, the reverse transcriptase domain of the retrotransposon gene modifying system is based on a reverse transcriptase domain of an APE-type or RLE-type non-LTR retrotransposon, or of a PLE-type retrotransposon. A wild-type reverse transcriptase domain of an APE-type, RLE-type, or PLE-type retrotransposon can be used in a retrotransposon gene modifying system or can be modified (e.g., by insertion, deletion, or substitution of one or more residues) to alter the reverse transcriptase activity for target DNA sequences. In some embodiments, the reverse transcriptase is altered from its natural sequence to have altered codon usage, e.g. improved for human cells. In some embodiments, the reverse transcriptase domain is a heterologous reverse transcriptase from a different LTR-retrotransposon, non-LTR retrotransposon, or other source. In certain embodiments, a retrotransposon gene modifying system includes a polypeptide that comprises a reverse transcriptase domain of a RTE (e.g., RTE-1_MD, RTE-3_BF, and RTE-25_LMi), CR1 (e.g., CR1-1_PH), Crack (e.g., Crack-28_RF), L2 (e.g., L2-2_Dre and L2-5_GA), and Vingi (e.g., Vingi-1_Acar) retrotransposon.

In certain embodiments, a retrotransposon gene modifying system includes a polypeptide that comprises a reverse transcriptase domain of a retrotransposon listed in Table 10, Table 11, Table X, Table Z1, Table Z2, or Table 3A or 3B of PCT Pub. No.: WO/2021/178717, which are incorporated herein by reference as they relate to domains from retrotransposons.

In certain embodiments, a retrotransposon gene modifying system includes a polypeptide that comprises a reverse transcriptase domain of a retrotransposon listed in Table R1. In some embodiments, the amino acid sequence of the reverse transcriptase domain of a retrotransposon gene modifying system is at least about 50%, at least about 60%, at least about 70%, at least about 80%, at least about 85%, at least about 90%, at least about 95%, at least about 96%, at least about 97%, at least about 98%, at least about 99% identical to the amino acid sequence of a reverse transcriptase domain of a retrotransposon whose DNA sequence is referenced in Table R1. Reverse transcriptase domains can be identified, for example, based upon homology to other known reverse transcription domains using routine tools as Basic Local Alignment Search Tool (BLAST). In some embodiments, reverse transcriptase domains are modified, for example by site-specific mutation. In some embodiments, the reverse transcriptase domain is engineered to bind a heterologous template RNA.

In some embodiments, a polypeptide (e.g., RT domain) comprises an RNA-binding domain, e.g., that specifically binds to an RNA sequence. In some embodiments, a template RNA comprises an RNA sequence that is specifically bound by the RNA-binding domain.

In some embodiments, the RT domain forms a dimer (e.g., a heterodimer or homodimer). In some embodiments, the RT domain is monomeric. In some embodiments, an RT domain naturally functions as a monomer or as a dimer (e.g., heterodimer or homodimer). In some embodiments, an RT domain naturally functions as a monomer. Naturally heterodimeric RT domains may, in some embodiments, also be functional as homodimers. In some embodiments, dimeric RT domains are expressed as fusion proteins, e.g., as homodimeric fusion proteins or heterodimeric fusion proteins. In some embodiments, the RT function of the system is fulfilled by multiple RT domains (e.g., as described herein). In further embodiments, the multiple RT domains are fused or separate, e.g., may be on the same polypeptide or on different polypeptides.

In some embodiment, a retrotransposon gene modifying polypeptide described herein comprises an RNase H domain, e.g., wherein the RNase H domain may be part of the RT domain. In some embodiments, an RT domain (e.g., as described herein) comprises an RNase H domain, e.g., an endogenous RNAse H domain or a heterologous RNase H domain. In some embodiments, an RT domain (e.g., as described herein) lacks an RNase H domain. In some embodiments, an RT domain (e.g., as described herein) comprises an RNase H domain that has been added, deleted, mutated, or swapped for a heterologous RNase H domain. In some embodiments, mutation of an RNase H domain yields a polypeptide exhibiting lower RNase activity, e.g., as determined by the methods described in Kotewicz et al. Nucleic Acids Res 16 (1): 265-277 (1988) (incorporated herein by reference in its entirety), e.g., lower by at least 10%, 20%, 30%, 40%, 50%, 60%, 70%, 80%, or 90% compared to an otherwise similar domain without the mutation. In some embodiments, RNase H activity is abolished.

In some embodiments, an RT domain is mutated to increase fidelity compared to an otherwise similar domain without the mutation. For instance, in some embodiments, a YADD (SEQ ID NO: 37635) or YMDD motif (SEQ ID NO: 37636) in an RT domain (e.g., in a reverse transcriptase) is replaced with YVDD (SEQ ID NO: 37637). In embodiments, replacement of the YADD (SEQ ID NO: 37635) or YMDD (SEQ ID NO: 37636) or YVDD (SEQ ID NO: 37637) results in higher fidelity in retroviral reverse transcriptase activity (e.g., as described in Jamburuthugoda and Eickbush J Mol Biol 2011; incorporated herein by reference in its entirety).

### Endonuclease Domain:

In some embodiments, the polypeptide comprises an endonuclease domain (e.g., a heterologous endonuclease domain). In certain embodiments, the endonuclease/DNA binding domain of an APE-type retrotransposon, the endonuclease domain of an RLE-type retrotransposon, or the endonuclease domain of a PLE-type retrotransposon can be used or can be modified (e.g., by insertion, deletion, or substitution of one or more residues) in a retrotransposon gene modifying system described herein. In some embodiments, the endonuclease domain or endonuclease/DNA binding domain is altered from its natural sequence to have altered codon usage, e.g. improved for human cells. In some embodiments, the endonuclease element is a heterologous endonuclease element. The amino acid sequence of an endonuclease domain of a retrotransposon gene modifying system described herein may be at least about 50%, at least about 60%, at least about 70%, at least about 80%, at least about 85%, at least about 90%, at least about 95%, at least about 96%, at least about 97%, at least about 98%, at least about 99% identical to the amino acid sequence of an endonuclease domain of a retrotransposon whose DNA sequence is referenced in Table X, Z1, Z2, 3A, or 3B of PCT Pub. No: WO/2021/178717, which are incorporated herein by reference as they relate to domains from retrotransposons.

In certain embodiments, a retrotransposon gene modifying system includes a polypeptide that comprises an endonuclease domain of a retrotransposon listed in Table R1. In some embodiments, the amino acid sequence of the endonuclease domain of a retrotransposon gene modifying system is at least about 50%, at least about 60%, at least about 70%, at least about 80%, at least about 85%, at least about 90%, at least about 95%, at least about 96%, at least about 97%, at least about 98%, at least about 99% identical to the amino acid sequence of a endonuclease domain of a retrotransposon whose DNA sequence is referenced in Table R1. Endonuclease domains can be identified, for example, based upon homology to other known endonuclease domains using tools as Basic Local Alignment Search Tool (BLAST).

In some embodiments, a retrotransposon gene modifying polypeptide possesses the function of DNA target site cleavage via an endonuclease domain. In some embodiments, the endonuclease domain is also a DNA-binding domain. In some embodiments, the endonuclease domain is also a template nucleic acid (e.g., template RNA) binding domain. In certain embodiments, the endonuclease/DNA binding domain of an APE-type retrotransposon or the endonuclease domain of an RLE-type retrotransposon can be used or can be modified (e.g., by insertion, deletion, or substitution of one or more residues) in a retrotransposon gene modifying system described herein.

### Template Nucleic Acid Binding Domain:

A retrotransposon gene modifying polypeptide typically contains regions capable of associating with the template nucleic acid (e.g., template RNA). In some embodiments, the template nucleic acid binding domain is an RNA binding domain. In some embodiments, the RNA binding domain is a modular domain that can associate with RNA molecules containing specific signatures, e.g., structural motifs, e.g., secondary structures present in the 3′ UTR in non-LTR retrotransposons. In other embodiments, the template nucleic acid binding domain (e.g., RNA binding domain) RNA binding domain is contained within the reverse transcription domain, e.g., the reverse transcriptase-derived component has a known signature for RNA preference, e.g., secondary structures present in the 3′ UTR in non-LTR retrotransposons.

### DNA Binding Domain:

In certain aspects, the DNA-binding domain of a retrotransposon gene modifying polypeptide described herein is selected, designed, or constructed for binding to a desired host DNA target sequence. In certain embodiments, the DNA-binding domain of the engineered retrotransposon is a heterologous DNA-binding protein or domain relative to a native retrotransposon sequence. In certain embodiments, the heterologous DNA-binding domain is a DNA binding domain of a retrotransposon described in Table R1 herein or in Table X, Table Z1, Table Z2, or Table 3A or 3B of PCT Pub. No.: WO/2021/178717. In some embodiments, DNA binding domains can be identified based upon homology to other known DNA binding domains using tools as Basic Local Alignment Search Tool (BLAST). In still other embodiments, DNA-binding domains are modified, for example by site-specific mutation. In some embodiments, the DNA binding domain is altered from its natural sequence to have altered codon usage, e.g. improved for human cells.

In embodiments, the DNA binding domain comprises one or more modifications relative to a wild-type DNA binding domain, e.g., a modification via directed evolution, e.g., phage-assisted continuous evolution (PACE).

In certain aspects of the present invention, the host DNA-binding site integrated into by the retrotransposon gene modifying system can be in a gene, in an intron, in an exon, an ORF, outside of a coding region of any gene, in a regulatory region of a gene, or outside of a regulatory region of a gene. In other aspects, the engineered retrotransposon may bind to one or more than one host DNA sequence. In other aspects, the engineered retrotransposon may have low sequence specificity, e.g., bind to multiple sequences or lack sequence preference.

In some embodiments, a retrotransposon gene modifying system is used to edit a target locus in multiple alleles. In some embodiments, a retrotransposon gene modifying system is designed to edit a specific allele. For example, a retrotransposon gene modifying polypeptide may be directed to a specific sequence that is only present on one allele, e.g., comprises a template RNA with homology to a target allele, e.g., an annealing domain, but not to a second cognate allele. In some embodiments, a retrotransposon gene modifying system can alter a haplotype-specific allele. In some embodiments, a retrotransposon gene modifying system that targets a specific allele preferentially targets that allele, e.g., has at least a 2, 4, 6, 8, or 10-fold preference for a target allele.

### Localization Sequences for Retrotransposon Gene Modifying Systems

In certain embodiments, a retrotransposon gene modifying system RNA further comprises an intracellular localization sequence, e.g., a nuclear localization sequence.

The nuclear localization sequence may be an RNA sequence that promotes the import of the RNA into the nucleus. In certain embodiments, the nuclear localization signal is located on the template RNA. In certain embodiments, the retrotransposase polypeptide is encoded on a first RNA, and the template RNA is a second, separate, RNA, and the nuclear localization signal is located on the template RNA and not on an RNA encoding the retrotransposase polypeptide. While not wishing to be bound by theory, in some embodiments, the RNA encoding the retrotransposase is targeted primarily to the cytoplasm to promote its translation, while the template RNA is targeted primarily to the nucleus to promote its retrotransposition into the genome. In some embodiments, the nuclear localization signal is at the 3′ end, 5′ end, or in an internal region of the template RNA. In some embodiments the nuclear localization signal is 3′ of the heterologous sequence (e.g., is directly 3′ of the heterologous sequence) or is 5′ of the heterologous sequence (e.g., is directly 5′ of the heterologous sequence). In some embodiments, the nuclear localization signal is placed outside of the 5′ UTR or outside of the 3′ UTR of the template RNA. In some embodiments the nuclear localization signal is placed between the 5′ UTR and the 3′ UTR, wherein optionally the nuclear localization signal is not transcribed with the transgene (e.g., the nuclear localization signal is an anti-sense orientation or is downstream of a transcriptional termination signal or polyadenylation signal). In some embodiments, the nuclear localization sequence is situated inside of an intron. In some embodiments a plurality of the same or different nuclear localization signals are in the RNA, e.g., in the template RNA. In some embodiments, the nuclear localization signal is less than 5, 10, 25, 50, 75, 100, 150, 200, 250, 300, 350, 400, 450, 500, 600, 700, 800, 900, or 1000 bp in length. Various RNA nuclear localization sequences can be used. For example, Lubelsky and Ulitsky, Nature 555 (107-111), 2018 describe RNA sequences, which drive RNA localization into the nucleus. In some embodiments, the nuclear localization signal is a SINE-derived nuclear RNA localization (SIRLOIN) signal. In some embodiments, the nuclear localization signal binds a nuclear-enriched protein. In some embodiments, the nuclear localization signal binds the HNRNPK protein. In some embodiments the nuclear localization signal is rich in pyrimidines, e.g., is a C/T rich, C/U rich, C rich, T rich, or U rich region. In some embodiments, the nuclear localization signal is derived from a long non-coding RNA. In some embodiments, the nuclear localization signal is derived from MALATI long non-coding RNA or is the 600 nucleotide M region of MALATI (described in Miyagawa et al., RNA 18, (738-751), 2012). In some embodiments, the nuclear localization signal is derived from BORG long non-coding RNA or is a AGCCC motif (described in Zhang et al., Molecular and Cellular Biology 34, 2318-2329 (2014). In some embodiments, the nuclear localization sequence is described in Shukla et al., The EMBO Journal e98452 (2018). In some embodiments, the nuclear localization signal is derived from a non-LTR retrotransposon, an LTR retrotransposon, retrovirus, or an endogenous retrovirus.

In some embodiments, a polypeptide described herein comprises one or more (e.g., 2, 3, 4, 5) nuclear targeting sequences, for example, a nuclear localization sequence (NLS), e.g., as described herein. In some embodiments, the NLS is a bipartite NLS. In some embodiments, an NLS facilitates the import of a protein comprising an NLS into the cell nucleus. In some embodiments, the NLS is fused to the N-terminus of a retrotransposon gene modifying polypeptide described herein. In some embodiments, the NLS is fused to the C-terminus of the retrotransposon gene modifying polypeptide. In some embodiments, a linker sequence is disposed between the NLS and the neighboring domain of the retrotransposon gene modifying polypeptide. In some embodiments, an NLS comprises the amino acid sequence of an NLS described herein.

In some embodiments, a nucleic acid described herein (e.g., an RNA encoding a retrotransposon gene modifying polypeptide, or a DNA encoding the RNA) comprises a microRNA binding site. In some embodiments, the microRNA binding site is used to increase the target-cell specificity of a retrotransposon gene modifying system. For instance, the microRNA binding site can be chosen on the basis that is recognized by a miRNA that is present in a non-target cell type, but that is not present (or is present at a reduced level relative to the non-target cell) in a target cell type. Thus, when the RNA encoding the retrotransposon gene modifying polypeptide is present in a non-target cell, it would be bound by the miRNA, and when the RNA encoding the retrotransposon gene modifying polypeptide is present in a target cell, it would not be bound by the miRNA (or bound but at reduced levels relative to the non-target cell). While not wishing to be bound by theory, binding of the miRNA to the RNA encoding the retrotransposon gene modifying polypeptide may reduce production of the retrotransposon gene modifying polypeptide, e.g., by degrading the mRNA encoding the polypeptide or by interfering with translation. Accordingly, the heterologous object sequence would be inserted into the genome of target cells more efficiently than into the genome of non-target cells. A system having a microRNA binding site in the RNA encoding the retrotransposon gene modifying polypeptide (or encoded in the DNA encoding the RNA) may also be used in combination with a template RNA that is regulated by a second microRNA binding site.

In some embodiments, a polypeptide for use in any of the systems described herein can be a molecular reconstruction or ancestral reconstruction based upon the aligned polypeptide sequence of multiple retrotransposons. In some embodiments, a 5′ or 3′ untranslated region for use in any of the systems described herein can be a molecular reconstruction based upon the aligned 5′ or 3′ untranslated region of multiple retrotransposons. Based on the Accession numbers, polypeptides or nucleic acid sequences can be aligned, e.g., by using routine sequence analysis tools as Basic Local Alignment Search Tool (BLAST) or CD-Search for conserved domain analysis. Molecular reconstructions can be created based upon sequence consensus, e.g. using approaches described in Ivics et al., Cell 1997, 501-510; Wagstaff et al., Molecular Biology and Evolution 2013, 88-99. In some embodiments, the retrotransposon from which the 5′ or 3′ untranslated region or polypeptide is derived is a young or a recently active mobile element, as assessed via phylogenetic methods such as those described in Boissinot et al., Molecular Biology and Evolution 2000, 915-928.

In some embodiments, a retrotransposon gene modifying system is capable of producing a substitution into the target site of at least 1, 2, 3, 4, 5, 6, 7, 8, 9, 10, 15, 20, 25, 30, 35, 40, 45, 50, 60, 70, 80, 90, or 100 or more nucleotides. In some embodiments, the substitution is a transition mutation. In some embodiments, the substitution is a transversion mutation. In some embodiments, the substitution converts an adenine to a thymine, an adenine to a guanine, an adenine to a cytosine, a guanine to a thymine, a guanine to a cytosine, a guanine to an adenine, a thymine to a cytosine, a thymine to an adenine, a thymine to a guanine, a cytosine to an adenine, a cytosine to a guanine, or a cytosine to a thymine.

In some embodiments, a retrotransposon gene modifying system is capable of producing an insertion into the target site of at least 45, 50, 55, 60, 65, 70, 75, 80, 85, 90, 95, or 100 nucleotides (and optionally no more than 500, 400, 300, 200, or 100 nucleotides). In some embodiments, a retrotransposon gene modifying system is capable of producing an insertion into the target site of at least 1, 2, 3, 4, 5, 6, 7, 8, 9, 10, 15, 20, 25, 30, 35, 40, 45, 50, 55, 60, 65, 70, 75, 80, 85, 90, 95, or 100 nucleotides (and optionally no more than 500, 400, 300, 200, or 100 nucleotides). In some embodiments, a retrotransposon gene modifying system is capable of producing an insertion into the target site of at least 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10 kilobases (and optionally no more than 1, 5, 10, or 20 kilobases). In some embodiments, a retrotransposon gene modifying system is capable of producing a deletion of at least 81, 85, 90, 95, 100, 110, 120, 130, 140, 150, 160, 170, 180, 190, or 200 nucleotides (and optionally no more than 500, 400, 300, or 200 nucleotides). In some embodiments, a retrotransposon gene modifying system is capable of producing a deletion of at least 81, 85, 90, 95, 100, 110, 120, 130, 140, 150, 160, 170, 180, 190, or 200 nucleotides (and optionally no more than 500, 400, 300, or 200 nucleotides). In some embodiments, a retrotransposon gene modifying system is capable of producing a deletion of at least 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200 nucleotides (and optionally no more than 500, 400, 300, or 200 nucleotides). In some embodiments, a retrotransposon gene modifying system is capable of producing a deletion of at least 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10 kilobases (and optionally no more than 1, 5, 10, or 20 kilobases).

In some embodiments, an insertion, deletion, substitution, or combination thereof, increases or decreases expression (e.g. transcription or translation) of a gene. In some embodiments, an insertion, deletion, substitution, or combination thereof, increases or decreases expression (e.g. transcription or translation) of a gene by altering, adding, or deleting sequences in a promoter or enhancer, e.g. sequences that bind transcription factors. In some embodiments, an insertion, deletion, substitution, or combination thereof alters translation of a gene (e.g. alters an amino acid sequence), inserts or deletes a start or stop codon, alters or fixes the translation frame of a gene. In some embodiments, an insertion, deletion, substitution, or combination thereof alters splicing of a gene, e.g. by inserting, deleting, or altering a splice acceptor or donor site. In some embodiments, an insertion, deletion, substitution, or combination thereof alters transcript or protein half-life. In some embodiments, an insertion, deletion, substitution, or combination thereof alters protein localization in the cell (e.g. from the cytoplasm to a mitochondria, from the cytoplasm into the extracellular space (e.g. adds a secretion tag)). In some embodiments, an insertion, deletion, substitution, or combination thereof alters (e.g. improves) protein folding (e.g. to prevent accumulation of misfolded proteins). In some embodiments, an insertion, deletion, substitution, or combination thereof, alters, increases, decreases the activity of a gene, e.g. a protein encoded by the gene.

In some embodiments, the retrotransposon gene modifying polypeptide results in insertion of the heterologous object sequence (e.g., the GFP gene) into the target locus (e.g., rDNA) at an average copy number of at least 0.01, 0.025, 0.05, 0.075, 0.1, 0.15, 0.2, 0.25, 0.3, 0.4, 0.5, 0.75, 1, 1.25, 1.5, 1.75, 2, 2.5, 3, 4, or 5 copies per genome. In some embodiments, a cell described herein (e.g., a cell comprising a heterologous sequence at a target insertion site) comprises the heterologous object sequence at an average copy number of at least 0.01, 0.025, 0.05, 0.075, 0.1, 0.15, 0.2, 0.25, 0.3, 0.4, 0.5, 0.75, 1, 1.25, 1.5, 1.75, 2, 2.5, 3, 4, or 5 copies per genome.

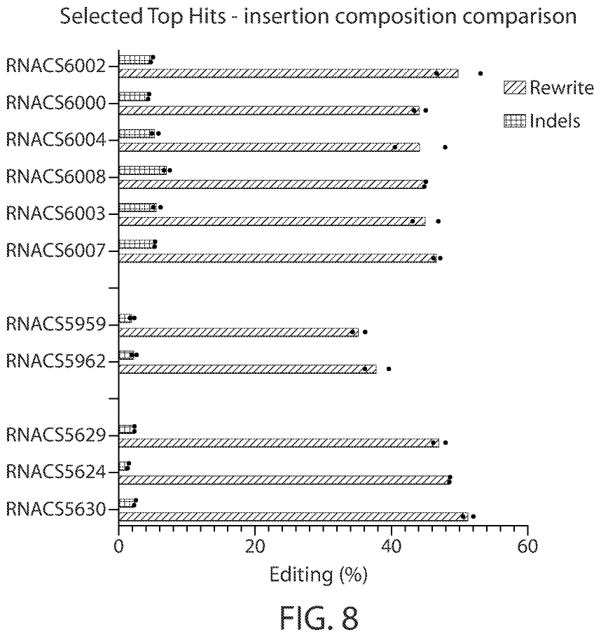

In some embodiments, a retrotransposon gene modifying system causes integration of a sequence in a target DNA with relatively few truncation events at the terminus. For instance, in some embodiments, a retrotransposon gene modifying protein results in about 25-100%, 50-100%, 60-100%, 70-100%, 75-95%, 80%-90%, or 86.17% of integrants into the target site being non-truncated, as measured by an assay described herein, e.g., an assay of Example 6 and FIG. 8 of PCT Application No. PCT/US2019/048607, incorporated herein by reference in its entirety. In some embodiments, a retrotransposon gene modifying protein results in at least about 30%, 40%, 50%, 60%, 70%, 80%, or 90% of integrants into the target site being non-truncated, as measured by an assay described herein. In some embodiments, an integrant is classified as truncated versus non-truncated using an assay comprising amplification with a forward primer situated 565 bp from the end of the element (e.g., a wild-type retrotransposon sequence) and a reverse primer situated in the genomic DNA of the target insertion site, e.g., rDNA. In some embodiments, the number of full-length integrants in the target insertion site is greater than the number of integrants truncated by 300-565 nucleotides in the target insertion site, e.g., the number of full-length integrants is at least 1.1×, 1.2×, 1.5×, 2×, 3×, 4×, 5×, 6×, 7×, 8×, 9×, or 10× the number of the truncated integrants, or the number of full-length integrants is at least 1.1×-10×, 2×-10×, 3×-10×, or 5×-10× the number of the truncated integrants.

In some embodiments, a system or method described herein results in insertion of the heterologous object sequence only at one target site in the genome of the target cell. Insertion can be measured, e.g., using a threshold of above 1%, 1.5%, 2%, 2.5%, 3%, 3.5%, 4%, 4.5%, 5%, e.g., as described in Example 8 of PCT Application No. PCT/US2019/048607, incorporated herein by reference in its entirety. In some embodiments, a system or method described herein results in insertion of the heterologous object sequence wherein less than 1%, 1.5%, 2%, 2.5%, 3%, 3.5%, 4%, 4.5%, 5%, 10%, 20%, 30%, 40%, or 50% of insertions are at a site other than the target site, e.g., using an assay described herein, e.g., an assay of Example 8 of PCT Application No. PCT/US2019/048607, incorporated herein by reference in its entirety.

In some embodiments, a system or method described herein results in “scarless” insertion of the heterologous object sequence, while in some embodiments, the target site can show deletions or duplications of endogenous DNA as a result of insertion of the heterologous sequence. The mechanisms of different retrotransposons could result in different patterns of duplications or deletions in the host genome occurring during retrotransposition at the target site. In some embodiments, the system results in a scarless insertion, with no duplications or deletions in the surrounding genomic DNA. In some embodiments, the system results in a deletion of less than 1, 2, 3, 4, 5, 10, 50, or 100 bp of genomic DNA upstream of the insertion. In some embodiments, the system results in a deletion of less than 1, 2, 3, 4, 5, 10, 50, or 100 bp of genomic DNA downstream of the insertion. In some embodiments, the system results in a duplication of less than 1, 2, 3, 4, 5, 10, 50, or 100 bp of genomic DNA upstream of the insertion. In some embodiments, the system results in a duplication of less than 1, 2, 3, 4, 5, 10, 50, or 100 bp of genomic DNA downstream of the insertion.

In some embodiments, a retrotransposon gene modifying system described herein, or a DNA-binding domain thereof, binds to its target site specifically, e.g., as measured using an assay of Example 21 of PCT Application No. PCT/US2019/048607, incorporated herein by reference in its entirety. In some embodiments, the retrotransposon gene modifying polypeptide or DNA-binding domain thereof binds to its target site more strongly than to any other binding site in the human genome. For example, in some embodiments, in an assay of Example 21 of PCT Application No. PCT/US2019/048607, incorporated herein by reference in its entirety, the target site represents more than 50%, 60%, 70%, 80%, 90%, or 95% of binding events of the retrotransposon gene modifying polypeptide or DNA-binding domain thereof to human genomic DNA. In some embodiments, the DNA binding domain of the retrotransposon gene modifying polypeptide is heterologous to the remainder of the retrotransposon gene modifying polypeptide, e.g., such that the retrotransposon gene modifying polypeptide targets a different target site that the endogenous DNA binding domain associated with the remainder of the retrotransposon gene modifying polypeptide.

### Genetically Engineered, e.g., Dimerized Retrotransposon Gene Modifying Polypeptides

Some non-LTR retrotransposons utilize two subunits to complete retrotransposition (Christensen et al PNAS 2006). In some embodiments, a retrotransposase described herein comprises two connected subunits as a single polypeptide. For instance, two wild-type retrotransposases could be joined with a linker to form a covalently “dimerized” protein. In some embodiments, the nucleic acid coding for the retrotransposase codes for two retrotransposase subunits to be expressed as a single polypeptide. In some embodiments, the subunits are connected by a peptide linker. Based on mechanism, not all functions are required from both retrotransposase subunits. In some embodiments, the fusion protein may consist of a fully functional subunit and a second subunit lacking one or more functional domains. In some embodiments, one subunit may lack reverse transcriptase functionality. In some embodiments, one subunit may lack the reverse transcriptase domain. In some embodiments, one subunit may possess only endonuclease activity. In some embodiments, one subunit may possess only an endonuclease domain. In some embodiments, the two subunits comprising the single polypeptide may provide complimentary functions.

In some embodiments, one subunit may lack endonuclease functionality. In some embodiments, one subunit may lack the endonuclease domain. In some embodiments, one subunit may possess only reverse transcriptase activity. In some embodiments, one subunit may possess only a reverse transcriptase domain. In some embodiments, one subunit may possess only DNA-dependent DNA synthesis functionality.

### Template RNA Component of Retrotransposon Gene Modifying System

The retrotransposon gene modifying systems described herein can transcribe an RNA sequence template into host target DNA sites by target-primed reverse transcription. By writing DNA sequence(s) via reverse transcription of the RNA sequence template directly into the host genome, the retrotransposon gene modifying system can insert an object sequence into a target genome without the need for exogenous DNA sequences to be introduced into the host cell (unlike, for example, CRISPR systems), as well as eliminate an exogenous DNA insertion step. Therefore, the retrotransposon gene modifying system provides a platform for the use of customized RNA sequence templates containing object sequences, e.g., sequences comprising heterologous gene coding and/or function information.

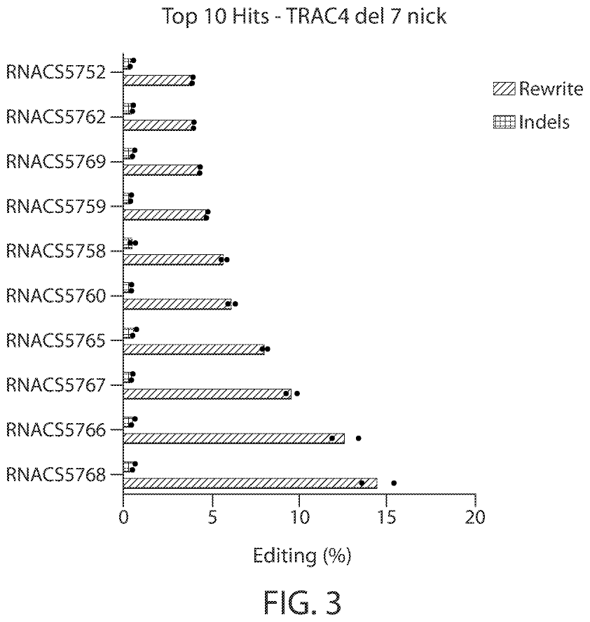

In some embodiments, the template RNA encodes a retrotransposon gene modifying protein in cis with a heterologous object sequence. Various cis constructs were described, for example, in Kuroki-Kami et al (2019) Mobile DNA 10:23 (incorporated by reference herein in its entirety), and can be used in combination with any of the embodiments described herein. For instance, in some embodiments, the template RNA comprises a heterologous object sequence, a sequence encoding a retrotransposon gene modifying protein (e.g., a protein comprising (i) a reverse transcriptase domain and (ii) an endonuclease domain, e.g., as described herein), a 5′ untranslated region, and a 3′ untranslated region. The components may be included in various orders. In some embodiments, the retrotransposon gene modifying protein and heterologous object sequence are encoded in different directions (sense vs. anti-sense), e.g., using an arrangement shown in FIG. 3A of Kuroki-Kami et al, Id. In some embodiments, the retrotransposon gene modifying protein and heterologous object sequence are encoded in the same direction. In some embodiments, the nucleic acid encoding the polypeptide and the template RNA or the nucleic acid encoding the template RNA are covalently linked, e.g., are part of a fusion nucleic acid, and/or are part of the same transcript. In some embodiments, the fusion nucleic acid comprises RNA or DNA.

The nucleic acid encoding the retrotransposon gene modifying polypeptide may, in some instances, be 5′ of the heterologous object sequence. For example, in some embodiments, the template RNA comprises, from 5′ to 3′, a 5′ untranslated region, a sense-encoded retrotransposon gene modifying polypeptide, a sense-encoded heterologous object sequence, and 3′ untranslated region. In some embodiments, the template RNA comprises, from 5′ to 3′, a 5′ untranslated region, a sense-encoded retrotransposon gene modifying polypeptide, anti-sense-encoded heterologous object sequence, and 3′ untranslated region.

It is understood that, when a template RNA is described as comprising an open reading frame or the reverse complement thereof, in some embodiments the template RNA must be converted into double stranded DNA (e.g., through reverse transcription) before the open reading frame can be transcribed and translated.

In certain embodiments, customized RNA sequence template can be identified, designed, engineered and constructed to contain sequences altering or specifying host genome function, for example by introducing a heterologous coding region into a genome; affecting or causing exon structure/alternative splicing; causing disruption of an endogenous gene; causing transcriptional activation of an endogenous gene; causing epigenetic regulation of an endogenous DNA; causing up- or down-regulation of operably liked genes, etc. In certain embodiments, a customized RNA sequence template can be engineered to contain sequences coding for exons and/or transgenes, provide for binding sites to transcription factor activators, repressors, enhancers, etc., and combinations of thereof. In other embodiments, the coding sequence can be further customized with splice acceptor sites, poly-A tails. In certain embodiments the RNA sequence can contain sequences coding for an RNA sequence template homologous to the retrotransposase, be engineered to contain heterologous coding sequences, or combinations thereof.

The template RNA may have some homology to the target DNA. In some embodiments the template RNA has at least 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 175, 200 or more bases of exact homology to the target DNA at the 3′ end of the RNA. In some embodiments the template RNA has at least 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 175, 180, or 200 or more bases of at least 50%, 60%, 70%, 80%, 85%, 90%, 95%, 97%, 98%, 99% or 100% homology to the target DNA, e.g., at the 5′ end of the template RNA. In some embodiments, the template RNA has a 3′ untranslated region derived from a retrotransposon, e.g. a retrotransposons described herein. In some embodiments the template RNA has a 3′ region of at least 10, 15, 20, 25, 30, 40, 50, 60, 80, 100, 120, 140, 160, 180, 200 or more bases of at least 50%, 60%, 70%, 80%, 85%, 90%, 95%, 97%, 98%, 99% or 100% homology to the 3′ sequence of a retrotransposon, e.g., a retrotransposon described herein, e.g. a retrotransposon in Table R1. In some embodiments, the template RNA has a 5′ untranslated region derived from a retrotransposon, e.g. a retrotransposons described herein. In some embodiments the template RNA has a 5′ region of at least 10, 15, 20, 25, 30, 40, 50, 60, 80, 100, 120, 140, 160, 180, or 200 or more bases of at least 40%, 50%, 60%, 70%, 80%, 90%, 95% or greater homology to the 5′ sequence of a retrotransposon, e.g., a retrotransposon described herein, e.g. a retrotransposon described in Table R1.

The template RNA component of a retrotransposon gene modifying system described herein typically is able to bind the retrotransposon gene modifying protein of the system. In some embodiments, the template RNA has a 3′ region that is capable of binding a retrotransposon gene modifying genome editing protein. The binding region, e.g., 3′ region, may be a structured RNA region, e.g., having at least 1, 2 or 3 hairpin loops, capable of binding the retrotransposon gene modifying protein of the system.

The template RNA component of a retrotransposon gene modifying system described herein typically is able to bind the retrotransposon gene modifying protein of the system. In some embodiments, the template RNA has a 5′ region that is capable of binding a retrotransposon gene modifying protein. The binding region, e.g., 5′ region, may be a structured RNA region, e.g., having at least 1, 2 or 3 hairpin loops, capable of binding the retrotransposon gene modifying protein of the system. In some embodiments, the 5′ untranslated region comprises a pseudoknot, e.g., a pseudoknot that is capable of binding to the retrotransposon gene modifying protein.

In some embodiments, the template RNA (e.g., an untranslated region of the hairpin RNA, e.g., a 5′ untranslated region) comprises a stem-loop sequence. In some embodiments, the template RNA (e.g., an untranslated region of the hairpin RNA, e.g., a 5′ untranslated region) comprises a hairpin. In some embodiments, the template RNA (e.g., an untranslated region of the hairpin RNA, e.g., a 5′ untranslated region) comprises a helix. In some embodiments, the template RNA (e.g., an untranslated region of the hairpin RNA, e.g., a 5′ untranslated region) comprises a psuedoknot. In some embodiments, the template RNA comprises a ribozyme. In some embodiments the ribozyme is similar to a hepatitis delta virus (HDV) ribozyme, e.g., has a secondary structure like that of the HDV ribozyme and/or has one or more activities of the HDV ribozyme, e.g., a self-cleavage activity. See, e.g., Eickbush et al., Molecular and Cellular Biology, 2010, 3142-3150.

In some embodiments, the template RNA (e.g., an untranslated region of the hairpin RNA, e.g., a 3′ untranslated region) comprises one or more stem-loops or helices. Exemplary structures of R2 3′ UTRs are shown, for example, in Ruschak et al. “Secondary structure models of the 3′ untranslated regions of diverse R2 RNAs” RNA. 2004 June; 10 (6): 978-987, e.g., at FIG. 3, therein, and in Eikbush and Eikbush, “R2 and R2/R1 hybrid non-autonomous retrotransposons derived by internal deletions of full-length elements” Mobile DNA (2012) 3:10; e.g., at FIG. 3 therein, which articles are hereby incorporated by reference in their entirety.

In some embodiments, a template RNA described herein comprises a sequence that is capable of binding to a retrotransposon gene modifying protein described herein. For instance, in some embodiments, the template RNA comprises an MS2 RNA sequence capable of binding to an MS2 coat protein sequence in the retrotransposon gene modifying protein. In some embodiments, the template RNA comprises an RNA sequence capable of binding to a B-box sequence. In some embodiments, in addition to or in place of a UTR, the template RNA is linked (e.g., covalently) to a non-RNA UTR, e.g., a protein or small molecule.

In some embodiments, the template RNA has a poly-A tail at the 3′ end. In some embodiments, the template RNA does not have a poly-A tail at the 3′ end.

In some embodiments the template RNA has a 5′ region of at least 10, 15, 20, 25, 30, 40, 50, 60, 80, 100, 120, 140, 160, 180, 200 or more bases of at least 40%, 50%, 60%, 70%, 80%, 90%, 95% or greater homology to the 5′ sequence of a retrotransposon, e.g., a retrotransposon described herein.

The template RNA of the system typically comprises an object sequence for insertion into a target DNA. The object sequence may be coding or non-coding.

In some embodiments, a system or method described herein comprises a single template RNA. In some embodiments, a system or method described herein comprises a plurality of template RNAs.

In some embodiments, the object sequence may contain an open reading frame. In some embodiments, the template RNA has a Kozak sequence. In some embodiments, the template RNA has an internal ribosome entry site. In some embodiments, the template RNA has a self-cleaving peptide such as a T2A or P2A site. In some embodiments, the template RNA has a start codon. In some embodiments, the template RNA has a splice acceptor site. In some embodiments, the template RNA has a splice donor site. Exemplary splice acceptor and splice donor sites are described in WO2016044416, incorporated herein by reference in its entirety. Exemplary splice acceptor site sequences are known to those of skill in the art and include, by way of example only, CTGACCCTTCTCTCTCTCCCCCAGAG (SEQ ID NO: 37661) (from human HBB gene) and TTTCTCTCCCACAAG (SEQ ID NO: 37662) (from human immunoglobulin-gamma gene). In some embodiments the template RNA, has a microRNA binding site downstream of the stop codon. In some embodiments, the template RNA has a polyA tail downstream of the stop codon of an open reading frame. In some embodiments, the template RNA comprises one or more exons. In some embodiments, the template RNA comprises one or more introns. In some embodiments, the template RNA comprises a eukaryotic transcriptional terminator. In some embodiments, the template RNA comprises an enhanced translation element or a translation enhancing element. In some embodiments, the RNA comprises the human T-cell leukemia virus (HTLV-1) R region. In some embodiments, the RNA comprises a posttranscriptional regulatory element that enhances nuclear export, such as that of Hepatitis B Virus (HPRE) or Woodchuck Hepatitis Virus (WPRE). In some embodiments, in the template RNA, the heterologous object sequence encodes a polypeptide and is coded in an antisense direction with respect to the 5′ and 3′ UTR. In some embodiments, in the template RNA, the heterologous object sequence encodes a polypeptide and is coded in a sense direction with respect to the 5′ and 3′ UTR.

In some embodiments, a nucleic acid described herein (e.g., a template RNA or a DNA encoding a template RNA) comprises a microRNA binding site. In some embodiments, the microRNA binding site is used to increase the target-cell specificity of a retrotransposon gene modifying system. For instance, the microRNA binding site can be chosen on the basis that is recognized by a miRNA that is present in a non-target cell type, but that is not present (or is present at a reduced level relative to the non-target cell) in a target cell type. Thus, when the template RNA is present in a non-target cell, it would be bound by the miRNA, and when the template RNA is present in a target cell, it would not be bound by the miRNA (or bound but at reduced levels relative to the non-target cell). While not wishing to be bound by theory, binding of the miRNA to the template RNA may interfere with insertion of the heterologous object sequence into the genome. Accordingly, the heterologous object sequence would be inserted into the genome of target cells more efficiently than into the genome of non-target cells. A system having a microRNA binding site in the template RNA (or DNA encoding it) may also be used in combination with a nucleic acid encoding a retrotransposon gene modifying polypeptide, wherein expression of the retrotransposon gene modifying polypeptide is regulated by a second microRNA binding site.

In some embodiments, the object sequence may contain a non-coding sequence. For example, the template RNA may comprise a promoter or enhancer sequence. In some embodiments, the template RNA comprises a tissue specific promoter or enhancer, each of which may be unidirectional or bidirectional. In some embodiments, the promoter is an RNA polymerase I promoter, RNA polymerase II promoter, or RNA polymerase III promoter. In some embodiments, the promoter comprises a TATA element. In some embodiments, the promoter comprises a B recognition element. In some embodiments, the promoter has one or more binding sites for transcription factors. In some embodiments, the non-coding sequence is transcribed in an antisense-direction with respect to the 5′ and 3′ UTR. In some embodiments, the non-coding sequence is transcribed in a sense direction with respect to the 5′ and 3′ UTR.

In some embodiments, a nucleic acid described herein (e.g., a template RNA or a DNA encoding a template RNA) comprises a promoter sequence, e.g., a tissue specific promoter sequence. In some embodiments, the tissue-specific promoter is used to increase the target-cell specificity of a retrotransposon gene modifying system. For instance, the promoter can be chosen on the basis that it is active in a target cell type but not active in (or active at a lower level in) a non-target cell type. Thus, even if the promoter integrated into the genome of a non-target cell, it would not drive expression (or only drive low-level expression) of an integrated gene. A system having a tissue-specific promoter sequence in the template RNA may also be used in combination with a microRNA binding site, e.g., in the template RNA or a nucleic acid encoding a retrotransposon gene modifying protein, e.g., as described herein. A system having a tissue-specific promoter sequence in the template RNA may also be used in combination with a DNA encoding a retrotransposon gene modifying polypeptide, driven by a tissue-specific promoter, e.g., to achieve higher levels of retrotransposon gene modifying protein in target cells than in non-target cells.

In some embodiments, a heterologous object sequence comprised by a template RNA (or DNA encoding the template RNA) is operably linked to at least one regulatory sequence. In some embodiments, the heterologous object sequence is operably linked to a tissue-specific promoter, such that expression of the heterologous object sequence, e.g., a therapeutic protein, is upregulated in target cells, as above. In some embodiments, the heterologous object sequence is operably linked to a miRNA binding site, such that expression of the heterologous object sequence, e.g., a therapeutic protein, is downregulated in cells with higher levels of the corresponding miRNA, e.g., non-target cells, as above.

In some embodiments, the template RNA comprises a microRNA sequence, a siRNA sequence, a guide RNA sequence, a piwi RNA sequence.

In some embodiments, the template RNA comprises a non-coding heterologous object sequence, e.g., a regulatory sequence. In some embodiments, integration of the heterologous object sequence thus alters the expression of an endogenous gene. In some embodiments, integration of the heterologous object sequence upregulates expression of an endogenous gene. In some embodiments, integration of the heterologous object sequence downregulated expression of an endogenous gene.

In some embodiments, the template RNA comprises a site that coordinates epigenetic modification. In some embodiments, the template RNA comprises an element that inhibits, e.g., prevents, epigenetic silencing. In some embodiments, the template RNA comprises a chromatin insulator. For example, the template RNA comprises a CTCF site or a site targeted for DNA methylation.

In order to promote higher level or more stable gene expression, the template RNA may include features that prevent or inhibit gene silencing. In some embodiments, these features prevent or inhibit DNA methylation. In some embodiments, these features promote DNA demethylation. In some embodiments, these features prevent or inhibit histone deacetylation. In some embodiments, these features prevent or inhibit histone methylation. In some embodiments, these features promote histone acetylation. In some embodiments, these features promote histone demethylation. In some embodiments, multiple features may be incorporated into the template RNA to promote one or more of these modifications. CpG dinculeotides are subject to methylation by host methyl transferases. In some embodiments, the template RNA is depleted of CpG dinucleotides, e.g., does not comprise CpG nucleotides or comprises a reduced number of CpG dinucleotides compared to a corresponding unaltered sequence. In some embodiments, the promoter driving transgene expression from integrated DNA is depleted of CpG dinucleotides.

In some embodiments, the template RNA comprises a gene expression unit composed of at least one regulatory region operably linked to an effector sequence. The effector sequence may be a sequence that is transcribed into RNA (e.g., a coding sequence or a non-coding sequence such as a sequence encoding a micro RNA).

In some embodiments, the object sequence of the template RNA is inserted into a target genome in an endogenous intron. In some embodiments, the object sequence of the template RNA is inserted into a target genome and thereby acts as a new exon. In some embodiments, the insertion of the object sequence into the target genome results in replacement of a natural exon or the skipping of a natural exon.

In some embodiments, the object sequence of the template RNA is inserted into the target genome in a genomic safe harbor site, such as AAVS1, CCR5, or ROSA26. In some embodiments, the object sequence of the template RNA is inserted into the albumin locus. In some embodiments, the object sequence of the template RNA is inserted into the TRAC locus. In some embodiments, the object sequence of the template RNA is added to the genome in an intergenic or intragenic region. In some embodiments, the object sequence of the template RNA is added to the genome 5′ or 3′ within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb of an endogenous active gene. In some embodiments, the object sequence of the template RNA is added to the genome 5′ or 3′ within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb of an endogenous promoter or enhancer. In some embodiments, the object sequence of the template RNA can be, e.g., 50-50,000 base pairs (e.g., between 50-40,000 bp, between 500-30,000 bp between 500-20,000 bp, between 100-15,000 bp, between 500-10,000 bp, between 50-10,000 bp, between 50-5,000 bp. In some embodiments, the heterologous object sequence is less than 1,000, 1,300, 1500, 2,000, 3,000, 4,000, 5,000, or 7,500 nucleotides in length.

The template nucleic acid (e.g., template RNA) component of a retrotransposon gene modifying system described herein typically is able to bind the retrotransposon gene modifying protein of the system. In some embodiments, the template nucleic acid (e.g., template RNA) has a 3′ region that is capable of binding a retrotransposon gene modifying protein. The binding region, e.g., 3′ region, may be a structured RNA region, e.g., having at least 1, 2 or 3 hairpin loops, capable of binding the retrotransposon gene modifying protein of the system. The binding region may associate the template nucleic acid (e.g., template RNA) with any of the polypeptide modules. In some embodiments, the binding region of the template nucleic acid (e.g., template RNA) may associate with an RNA-binding domain in the polypeptide. In some embodiments, the binding region of the template nucleic acid (e.g., template RNA) may associate with the reverse transcription domain of the polypeptide (e.g., specifically bind to the RT domain). For example, where the reverse transcription domain is derived from a non-LTR retrotransposon, the template nucleic acid (e.g., template RNA) may contain a binding region derived from a non-LTR retrotransposon, e.g., a 3′ UTR from a non-LTR retrotransposon. In some embodiments a system or method described herein comprises a single template nucleic acid (e.g., template RNA). In some embodiments a system or method described herein comprises a plurality of template nucleic acids (e.g., template RNAs). In some embodiments, when the system comprises a plurality of nucleic acids, each nucleic acid comprises a conjugating domain. In some embodiments, a conjugating domain enables association of nucleic acid molecules, e.g., by hybridization of complementary sequences.

In some embodiments, the template nucleic acid may comprise one or more UTRs (e.g., a 5′ UTR or a 3′ UTR, e.g., from an R2-type retrotransposon). In some embodiments, the UTR facilitates interaction of the template with the reverse transcriptase domain of the polypeptide. In some embodiments, the template possesses one or more sequences aiding in association of the template with the retrotransposon gene modifying polypeptide. In some embodiments, these sequences may be derived from retrotransposon UTRs. In some embodiments, the UTRs may be located flanking the desired insertion sequence. In some embodiments, a sequence with target site homology may be located outside of one or both UTRs. In some embodiments, the sequence with target site homology can anneal to the target sequence to prime reverse transcription. In some embodiments, the 5′ and/or 3′ UTR may be located terminal to the target site homology sequence. In some embodiments, the retrotransposon gene modifying system may result in the insertion of a desired payload without any additional sequence (e.g., a gene expression unit without UTRs used to bind the retrotransposon gene modifying protein).

In some embodiments, the template RNA comprises one or more of (e.g., 2, 3, 4, or all of) the following sequences from 5′ to 3′: 5′ UTR, bGHpA, an object sequence for incorporation into the genome, Kozak sequence, promoter (e.g., EF1a short promoter), 3′ UTR.

In some embodiments, the template RNA comprises a 5′ UTR. In some embodiments, the template RNA comprises bGHpA. In some embodiments, the template RNA comprises a Kozak sequence. In some embodiments, the template RNA comprises a promoter (e.g., an EF1a short promoter). In some embodiments, the template RNA comprises a 3′ UTR.

In some embodiments, the template RNA comprises one or more of the following sequences from 5′ to 3′: 5′ UTR, TKpA, an object sequence for incorporation into the genome, Kozak sequence, promoter (e.g., an EF1a short promoter or MND promoter), 3′ UTR. In some embodiments, the promoter comprises all or a functional portion of an EF1a promoter sequence. In some embodiments, the promoter comprises all or a functional portion of an MND promoter sequence.

In some embodiments, the template RNA comprises a 5′ UTR. In some embodiments, the template RNA comprises TKpA. In some embodiments, the template RNA comprises a Kozak sequence. In some embodiments, the template RNA comprises a promoter, e.g., an EF1a short promoter. In some embodiments, the template RNA comprises an MND promoter. In some embodiments, the template RNA comprises a 3′ UTR.

The template nucleic acid (e.g., template RNA) can be designed to result in insertions, mutations, or deletions at the target DNA locus. In some embodiments, the template nucleic acid (e.g., template RNA) may be designed to cause an insertion in the target DNA. For example, the template nucleic acid (e.g., template RNA) may contain a heterologous sequence, wherein the reverse transcription will result in insertion of the heterologous sequence into the target DNA. In other embodiments, the RNA template may be designed to write a deletion into the target DNA. For example, the template nucleic acid (e.g., template RNA) may match the target DNA upstream and downstream of the desired deletion, wherein the reverse transcription will result in the copying of the upstream and downstream sequences from the template nucleic acid (e.g., template RNA) without the intervening sequence, e.g., causing deletion of the intervening sequence. In other embodiments, the template nucleic acid (e.g., template RNA) may be designed to write an edit into the target DNA. For example, the template RNA may match the target DNA sequence with the exception of one or more nucleotides, wherein the reverse transcription will result in the copying of these edits into the target DNA, e.g., resulting in mutations, e.g., transition or transversion mutations.

In some embodiments, a retrotransposon gene modifying system is capable of producing an insertion into the target site of at least 45, 50, 55, 60, 65, 70, 75, 80, 85, 90, 95, or 100 nucleotides (and optionally no more than 500, 400, 300, 200, or 100 nucleotides). In some embodiments, a retrotransposon gene modifying system is capable of producing an insertion into the target site of at least 1, 2, 3, 4, 5, 6, 7, 8, 9, 10, 15, 20, 25, 30, 35, 40, 45, 50, 55, 60, 65, 70, 75, 80, 85, 90, 95, or 100 nucleotides (and optionally no more than 500, 400, 300, 200, or 100 nucleotides). In some embodiments, a retrotransposon gene modifying system is capable of producing an insertion into the target site of at least 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10 kilobases (and optionally no more than 1, 5, 10, or 20 kilobases). In some embodiments, a retrotransposon gene modifying system is capable of producing a deletion of at least 81, 85, 90, 95, 100, 110, 120, 130, 140, 150, 160, 170, 180, 190, or 200 nucleotides (and optionally no more than 500, 400, 300, or 200 nucleotides). In some embodiments, a retrotransposon gene modifying system is capable of producing a deletion of at least 81, 85, 90, 95, 100, 110, 120, 130, 140, 150, 160, 170, 180, 190, or 200 nucleotides (and optionally no more than 500, 400, 300, or 200 nucleotides). In some embodiments, a retrotransposon gene modifying system is capable of producing a deletion of at least 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200 nucleotides (and optionally no more than 500, 400, 300, or 200 nucleotides). In some embodiments, a retrotransposon gene modifying system is capable of producing a deletion of at least 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10 kilobases (and optionally no more than 1, 5, 10, or 20 kilobases).

### Additional Template Features

In some embodiments, the template (e.g., template RNA) comprises certain structural features, e.g., determined in silico. In embodiments, the template RNA is predicted to have minimal energy structures between −280 and −480 kcal/mol (e.g., between −280 to −300, −300 to −350, −350 to −400, −400 to −450, or −450 to −480 kcal/mol), e.g., as measured by RNAstructure, e.g., as described in Turner and Mathews Nucleic Acids Res 38: D280-282 (2009) (incorporated herein by reference in its entirety).

In some embodiments, the template (e.g., template RNA) comprises certain structural features, e.g., determined in vitro. In embodiments, the template RNA is sequence optimized, e.g., to reduce secondary structure as determined in vitro, for example, by SHAPE-MaP (e.g., as described in Siegfried et al. Nat Methods 11:959-965 (2014); incorporated herein by reference in its entirety). In some embodiments, the template (e.g., template RNA) comprises certain structural features, e.g., determined in cells. In embodiments, the template RNA is sequence optimized, e.g., to reduce secondary structure as measured in cells, for example, by DMS-MaPseq (e.g., as described in Zubradt et al. Nat Methods 14:75-82 (2017); incorporated by reference herein in its entirety).

### Additional Components and Properties of Retrotransposon Gene Modifying Systems

A retrotransposon gene modifying system may further comprises one or more components described herein in the context of heterologous gene modifying systems. Additional details regarding these components and accompanying references to sections providing additional details are provided below.

As disclosed herein, a retrotransposon gene modifying system RNA may further comprise an intracellular localization sequence. Additional details are provided in the section entitled “Localization sequences for heterologous gene modifying systems.” In some embodiments, the retrotransposon gene modifying system comprises an intein. Additional details are provided in the section entitled “Inteins.” In some embodiments, a retrotransposon gene modifying polypeptide may comprise a linker, e.g., a peptide linker, e.g., a linker as described in Table 10. Additional details are provided in the section entitled “Linkers”. In some embodiments, a nucleic acid encoding a retrotransposon gene modifying polypeptide may comprise a promoter or enhancer. Additional details are provided in the section entitled “Promoters.” In some embodiments, the invention provides evolved variants of retrotransposon gene modifying polypeptides as described herein, e.g., in the section entitled “Evolved Variants of Heterologous Gene Modifying Polypeptides and Systems.”

A retrotransposon gene modifying system as described herein may, in some instances, be characterized by one or more functional measurements or characteristics. In some embodiments, the DNA binding domain has one or more of the functional characteristics described below. In some embodiments, the RNA binding domain has one or more of the functional characteristics described below. In some embodiments, the endonuclease domain has one or more of the functional characteristics described below. In some embodiments, the reverse transcriptase domain has one or more of the functional characteristics described below. In some embodiments, the template (e.g., template RNA) has one or more of the functional characteristics described below. In some embodiments, the target site bound by the retrotransposon gene modifying polypeptide has one or more of the functional characteristics described below.

### Retrotransposon Gene Modifying Polypeptide

### DNA Binding Domain

In some embodiments, the DNA binding domain is capable of binding to a target sequence (e.g., a dsDNA target sequence) with greater affinity than a reference DNA binding domain. In some embodiments, the reference DNA binding domain is a DNA binding domain from R2 BM of B. mori. In some embodiments, the DNA binding domain is capable of binding to a target sequence (e.g., a dsDNA target sequence) with an affinity between 100 μM-10 nM (e.g., between 100 μM-1 nM or 1 nM-10 nM).

In some embodiments, the affinity of a DNA binding domain for its target sequence (e.g., dsDNA target sequence) is measured in vitro, e.g., by thermophoresis, e.g., as described in Asmari et al. Methods 146:107-119 (2018) (incorporated by reference herein in its entirety).

In embodiments, the DNA binding domain is capable of binding to its target sequence (e.g., dsDNA target sequence), e.g, with an affinity between 100 μM-10 nM (e.g., between 100 pM-1 nM or 1 nM-10 nM) in the presence of a molar excess of scrambled sequence competitor dsDNA, e.g., of about 100-fold molar excess.

In some embodiments, the DNA binding domain is found associated with its target sequence (e.g., dsDNA target sequence) more frequently than any other sequence in the genome of a target cell, e.g., human target cell, e.g., as measured by ChIP-seq (e.g., in HEK293T cells), e.g., as described in He and Pu (2010) Curr. Protoc Mol Biol Chapter 21 (incorporated herein by reference in its entirety). In some embodiments, the DNA binding domain is found associated with its target sequence (e.g., dsDNA target sequence) at least about 5-fold or 10-fold, more frequently than any other sequence in the genome of a target cell, e.g., as measured by ChIP-seq (e.g., in HEK293T cells), e.g., as described in He and Pu (2010), supra.

In some embodiments, a retrotransposon gene modifying polypeptide comprises a modification to a DNA-binding domain, e.g., relative to the wild-type polypeptide. In some embodiments, the DNA-binding domain comprises an addition, deletion, replacement, or modification to the amino acid sequence of the original DNA-binding domain. In some embodiments, the DNA-binding domain is modified to include a heterologous functional domain that binds specifically to a target nucleic acid (e.g., DNA) sequence of interest. In some embodiments, the functional domain replaces at least a portion (e.g., the entirety of) the prior DNA-binding domain of the polypeptide. In some embodiments, a retrotransposon gene modifying polypeptide comprises a modification to an endonuclease domain, e.g., relative to the wild-type polypeptide. In some embodiments, the endonuclease domain comprises an addition, deletion, replacement, or modification to the amino acid sequence of the original endonuclease domain. In some embodiments, the endonuclease domain is modified to include a heterologous functional domain that binds specifically to and/or induces endonuclease cleavage of a target nucleic acid (e.g., DNA) sequence of interest.

### RNA Binding Domain

In some embodiments, the RNA binding domain is capable of binding to a template RNA with greater affinity than a reference RNA binding domain. In some embodiments, the reference RNA binding domain is an RNA binding domain from R2_BM of B. mori. In some embodiments, the RNA binding domain is capable of binding to a template RNA with an affinity between 100 μM-10 nM (e.g., between 100 μM-1 nM or 1 nM-10 nM). In some embodiments, the affinity of a RNA binding domain for its template RNA is measured in vitro, e.g., by thermophoresis, e.g., as described in Asmari et al. Methods 146:107-119 (2018) (incorporated by reference herein in its entirety). In some embodiments, the affinity of a RNA binding domain for its template RNA is measured in cells (e.g., by FRET or CLIP-Seq).

In some embodiments, the RNA binding domain is associated with the template RNA in vitro at a frequency at least about 5-fold or 10-fold higher than with a scrambled RNA. In some embodiments, the frequency of association between the RNA binding domain and the template RNA or scrambled RNA is measured by CLIP-seq, e.g., as described in Lin and Miles (2019) Nucleic Acids Res 47 (11): 5490-5501 (incorporated by reference herein in its entirety). In some embodiments, the RNA binding domain is associated with the template RNA in cells (e.g., in HEK293T cells) at a frequency at least about 5-fold or 10-fold higher than with a scrambled RNA. In some embodiments, the frequency of association between the RNA binding domain and the template RNA or scrambled RNA is measured by CLIP-seq, e.g., as described in Lin and Miles (2019), supra.

### Endonuclease Domain

In some embodiments, the endonuclease domain is associated with the target dsDNA in vitro at a frequency at least about 5-fold or 10-fold higher than with a scrambled dsDNA. In some embodiments, the endonuclease domain is associated with the target dsDNA in vitro at a frequency at least about 5-fold or 10-fold higher than with a scrambled dsDNA, e.g., in a cell (e.g., a HEK293T cell). In some embodiments, the frequency of association between the endonuclease domain and the target DNA or scrambled DNA is measured by ChIP-seq, e.g., as described in He and Pu (2010) Curr. Protoc Mol Biol Chapter 21 (incorporated by reference herein in its entirety).

In some embodiments, the endonuclease domain can catalyze the formation of a nick at a target sequence, e.g., to an increase of at least about 5-fold or 10-fold relative to a non-target sequence (e.g., relative to any other genomic sequence in the genome of the target cell). In some embodiments, the level of nick formation is determined using NickSeq, e.g., as described in Elacqua et al. (2019) bioRxiv doi.org/10.1101/867937 (incorporated herein by reference in its entirety).

In some embodiments, the endonuclease domain is capable of nicking DNA in vitro. In embodiments, the nick results in an exposed base. In embodiments, the exposed base can be detected using a nuclease sensitivity assay, e.g., as described in Chaudhry and Weinfeld (1995) Nucleic Acids Res 23 (19): 3805-3809 (incorporated by reference herein in its entirety). In embodiments, the level of exposed bases (e.g., detected by the nuclease sensitivity assay) is increased by at least 10%, 50%, or more relative to a reference endonuclease domain. In some embodiments, the reference endonuclease domain is an endonuclease domain from R2 BM of B. mori.

In some embodiments, the endonuclease domain is capable of nicking DNA in a cell. In embodiments, the endonuclease domain is capable of nicking DNA in a HEK293T cell. In embodiments, an unrepaired nick that undergoes replication in the absence of Rad51 results in increased NHEJ rates at the site of the nick, which can be detected, e.g., by using a Rad51 inhibition assay, e.g., as described in Bothmer et al. (2017) Nat Commun 8:13905 (incorporated by reference herein in its entirety). In embodiments, NHEJ rates are increased above 0-5%. In embodiments, NHEJ rates are increased to 20-70% (e.g., between 30%-60% or 40-50%), e.g., upon Rad51 inhibition.

In some embodiments, the endonuclease domain releases the target after cleavage. In some embodiments, release of the target is indicated indirectly by assessing for multiple turnovers by the enzyme, e.g., as described in Yourik at al. RNA 25 (1): 35-44 (2019) (incorporated herein by reference in its entirety) and shown in FIG. 2. In some embodiments, the kₑₓₚ of an endonuclease domain is 1×10⁻³-1×10⁻⁵ min-1 as measured by such methods.

In some embodiments, the endonuclease domain has a catalytic efficiency (k_{cat}/Kₘ) greater than about 1×10⁸ s⁻¹ M⁻¹ in vitro. In embodiments, the endonuclease domain has a catalytic efficiency greater than about 1×10⁵, 1×10⁶, 1×10⁷, or 1×10⁸, s⁻¹ M⁻¹ in vitro. In embodiments, catalytic efficiency is determined as described in Chen et al. (2018) Science 360 (6387): 436-439 (incorporated herein by reference in its entirety). In some embodiments, the endonuclease domain has a catalytic efficiency (k_{cat}/Kₘ) greater than about 1×10⁸ s⁻¹ M⁻¹ in cells. In embodiments, the endonuclease domain has a catalytic efficiency greater than about 1×10⁵, 1×10⁶, 1×10⁷, or 1×10⁸ s⁻¹ M⁻¹ in cells.

### Reverse Transcriptase Domain

In some embodiments, the reverse transcriptase domain has a lower probability of premature termination rate (P_{off}) in vitro relative to a reference reverse transcriptase domain. In some embodiments, the reference reverse transcriptase domain is a reverse transcriptase domain from R2 BM of B. mori or a viral reverse transcriptase domain, e.g., the RT domain from M-MLV.

In some embodiments, the reverse transcriptase domain has a lower probability of premature termination rate (P_{off}) in vitro of less than about 5×10⁻³/nt, 5×10⁻⁴/nt, or 5×10⁻⁶/nt, e.g., as measured on a 1094 nt RNA. In embodiments, the in vitro premature termination rate is determined as described in Bibillo and Eickbush (2002) J Biol Chem 277 (38): 34836-34845 (incorporated by reference herein its entirety).

In some embodiments, the reverse transcriptase domain is able to complete at least about 30% or 50% of integrations in cells. The percent of complete integrations can be measured by dividing the number of substantially full-length integration events (e.g., genomic sites that comprise at least 98% of the expected integrated sequence) by the number of total (including substantially full-length and partial) integration events in a population of cells. In embodiments, the integrations in cells is determined (e.g., across the integration site) using long-read amplicon sequencing, e.g., as described in Karst et al. (2020) bioRxiv doi.org/10.1101/645903 (incorporated by reference herein its in entirety).

In embodiments, quantifying integrations in cells comprises counting the fraction of integrations that contain at least about 75%, 80%, 85%, 90%, 95%, 96%, 97%, 98%, 99%, or 100% of the DNA sequence corresponding to the template RNA (e.g., a template RNA having a length of at least 0.05, 0.1, 0.5, 0.6, 0.7, 0.8, 0.9, 1, 1.5, 2, 3, 4, or 5 kb, e.g., a length between 0.5-0.6, 0.6-0.7, 0.7-0.8, 0.8-0.9, 1.0-1.2, 1.2-1.4, 1.4-1.6, 1.6-1.8, 1.8-2.0, 2-3, 3-4, or 4-5 kb).

In some embodiments, the reverse transcriptase domain is capable of polymerizing dNTPs in vitro. In embodiments, the reverse transcriptase domain is capable of polymerizing dNTPs in vitro at a rate between 0.1-50 nt/sec (e.g., between 0.1-1, 1-10, or 10-50 nt/sec). In embodiments, polymerization of dNTPs by the reverse transcriptase domain is measured by a single-molecule assay, e.g., as described in Schwartz and Quake (2009) PNAS 106 (48): 20294-20299 (incorporated by reference in its entirety).

In some embodiments, the reverse transcriptase domain has an in vitro error rate (e.g., misincorporation of nucleotides) of between 1×10⁻³-1×10⁻⁴ or 1×10⁻⁴-1×10⁻⁵ substitutions/nt, e.g., as described in Yasukawa et al. (2017) Biochem Biophys Res Commun 492 (2): 147-153 (incorporated herein by reference in its entirety). In some embodiments, the reverse transcriptase domain has an error rate (e.g., misincorporation of nucleotides) in cells (e.g., HEK293T cells) of between 1×10⁻³-1×10⁻⁴ or 1×10⁻⁴-1×10⁻⁵ substitutions/nt, e.g., by long-read amplicon sequencing, e.g., as described in Karst et al. (2020) bioRxiv doi.org/10.1101/645903 (incorporated by reference herein in its entirety).

In some embodiments, the reverse transcriptase domain is capable of performing reverse transcription of a target RNA in vitro. In some embodiments, the reverse transcriptase requires a primer of at least 3 nt to initiate reverse transcription of a template. In some embodiments, reverse transcription of the target RNA is determined by detection of cDNA from the target RNA (e.g., when provided with a ssDNA primer, e.g., which anneals to the target with at least 3, 4, 5, 6, 7, 8, 9, or 10 nt at the 3′ end), e.g., as described in Bibillo and Eickbush (2002) J Biol Chem 277 (38): 34836-34845 (incorporated herein by reference in its entirety).

In some embodiments, the reverse transcriptase domain performs reverse transcription at least 5 or 10 times more efficiently (e.g., by cDNA production), e.g., when converting its RNA template to cDNA, for example, as compared to an RNA template lacking the protein binding motif (e.g., a 3′ UTR). In embodiments, efficiency of reverse transcription is measured as described in Yasukawa et al. (2017) Biochem Biophys Res Commun 492 (2): 147-153 (incorporated by reference herein in its entirety).

In some embodiments, the reverse transcriptase domain specifically binds a specific RNA template with higher frequency (e.g., about 5 or 10-fold higher frequency) than any endogenous cellular RNA, e.g., when expressed in cells (e.g., HEK293T cells). In embodiments, frequency of specific binding between the reverse transcriptase domain and the template RNA are measured by CLIP-seq, e.g., as described in Lin and Miles (2019) Nucleic Acids Res 47 (11): 5490-5501 (incorporated herein by reference in its entirety).

### Target Site and Integration

In some embodiments, after gene editing, the target site surrounding the integrated sequence contains a limited number of insertions or deletions, for example, in less than about 50% or 10% of integration events, e.g., as determined by long-read amplicon sequencing of the target site, e.g., as described in Karst et al. (2020) bioRxiv doi.org/10.1101/645903 (incorporated by reference herein in its entirety). In some embodiments, the target site does not show multiple insertion events, e.g., head-to-tail or head-to-head duplications, e.g., as determined by long-read amplicon sequencing of the target site, e.g., as described in Karst et al. bioRxiv doi.org/10.1101/645903 (2020) (incorporated herein by reference in its entirety). In some embodiments, the target site contains an integrated sequence corresponding to the template RNA. In some embodiments, the target site does not contain insertions resulting from endogenous RNA in more than about 1% or 10% of events, e.g., as determined by long-read amplicon sequencing of the target site, e.g., as described in Karst et al. bioRxiv doi.org/10.1101/645903 (2020) (incorporated herein by reference in its entirety). In some embodiments, the target site contains the integrated sequence corresponding to the template RNA.

In some embodiments, the target site contains an integrated sequence corresponding to the template RNA. In embodiments, the target site does not comprise sequence outside of the template, e.g., as determined by long-read amplicon sequencing of the target site (for example, as described in Karst et al. bioRxiv doi.org/10.1101/645903 (2020); incorporated herein by reference in its entirety).

In some embodiments, the heterologous object sequence is integrated upstream of a gene (e.g., within 2 kb or 10 kb of a transcription start site (TSS)), within a coding portion of a gene (e.g., an exon), within a non-coding portion of a gene (e.g., an intron), or an intergenic location (e.g., downstream of a gene). In some embodiments, within a plurality of cells containing a plurality of copies of a gene encoded by the heterologous object sequence, less than or equal to 70%, 65%, 60%, or 55% of copies of the heterologous object sequence (e.g., gene encoding a CAR) are situated within a gene endogenous to a cell of the population. In some embodiments, less than 10%, 9%, 8%, 7%, 6%, or 5% of copies of the heterologous object sequence (e.g., gene encoding a CAR) are situated within an exon of a gene endogenous to a cell of the population. In some embodiments, less than 10%, 9%, 8%, 7%, 6%, or 5% of copies of the heterologous object sequence (e.g., gene encoding a CAR) are situated within a coding region of a gene endogenous to a cell of the population. In some embodiments, less than 70%, 65%, 60%, 55%, or 50% of copies of the heterologous object sequence (e.g., gene encoding a CAR) are situated within an intron of a gene endogenous to a cell of the population. In some embodiments, less than 70%, 65%, 60%, 55%, or 50% of copies of the heterologous object sequence (e.g., gene encoding a CAR) are situated within a non-coding region of a gene endogenous to a cell of the population. In some embodiments, less than 10%, 9%, 8%, or 7% of copies of the heterologous object sequence (e.g., gene encoding a CAR) are situated upstream of a gene (e.g., within 2 kb of a transcriptional start site (TSS)) endogenous to a cell of the population. In some embodiments, at least 20%, 25%, 30%, 35%, or 40% of copies of the heterologous object sequence (e.g., gene encoding a CAR) are situated within an intergenic region endogenous to a cell of the population. In some embodiments, at least 20%, 25%, 30%, 35%, or 40% of copies of the heterologous object sequence (e.g., gene encoding a CAR) are situated downstream of a gene endogenous to a cell of the population.

In some embodiments, modifying a genome of a cell using a retrotransposon gene modifying system described herein results in a higher level of insertions in an intergenic location compared to a lentiviral system. Without wishing to be bound by theory, the integration pattern of the retrotransposon gene modifying systems is advantageous because, in some embodiments, it is desired to reduce disrupting expression of endogenous genes in the host cell.

### DNA Damage Response

In some embodiments, modifying a genome of a cell (e.g., a primary cell, e.g., a T cell or an IPSC) using a retrotransposon gene modifying system does not result in activation of the endogenous DNA damage response (DDR) pathway. In some embodiments, modifying a genome of a cell (e.g., a primary cell, e.g., an IPSC) using a retrotransposon gene modifying system results in activation of the cell's endogenous DDR pathway less than in an otherwise similar cell treated with Cas9.

In some embodiments, modifying a genome of a cell (e.g., a primary cell, e.g., a T cell or an IPSC) using a retrotransposon gene modifying system does not result in activation of the endogenous interferon response. In some embodiments, modifying a genome of a cell (e.g., a primary cell, e.g., a T cell or an IPSC) using a retrotransposon gene modifying system results in activation of the cell's interferon response less than in an otherwise similar cell treated with a gene modifying system comprising elements from a LINE-1 retrotransposase.

In some embodiments, the retrotransposon gene modifying polypeptide systems described herein includes a self-inactivating module. The self-inactivating module leads to a decrease of expression of the retrotransposon gene modifying polypeptide, the retrotransposon gene modifying template, or both. Self-inactivating modules are described, e.g., at p. 1200-1201 of PCT Pub. No. WO/2021/178720, which are herein incorporated by reference in their entirety.

In some embodiments a polypeptide described herein (e.g., a retrotransposon gene modifying polypeptide) is controllable via a small molecule. In some embodiments, the polypeptide is dimerized via a small molecule. Polypeptides of this type are described, e.g., at p. 1201-1203 of WO/2021/178720, which are herein incorporated by reference in their entirety.

### Circular RNAs and Ribozymes in Gene Modifying Systems

It is contemplated that it may be useful to employ circular and/or linear RNA states during the formulation, delivery, or gene modifying reaction within the target cell. Thus, in some embodiments of any of the aspects described herein, a heterologous or retrotransposon gene modifying system comprises one or more circular RNAs (circRNAs). In some embodiments of any of the aspects described herein, a gene modifying system comprises one or more linear RNAs. In some embodiments, a nucleic acid as described herein (e.g., a template nucleic acid, a nucleic acid molecule encoding a gene modifying polypeptide, or both) is a circRNA. In some embodiments, a circular RNA molecule encodes the gene modifying polypeptide. In some embodiments, the circRNA molecule encoding the gene modifying polypeptide is delivered to a host cell. In some embodiments, a circular RNA molecule encodes a recombinase, e.g., as described herein. In some embodiments, the circRNA molecule encoding the recombinase is delivered to a host cell. In some embodiments, the circRNA molecule encoding the gene modifying polypeptide is linearized (e.g., in the host cell, e.g., in the nucleus of the host cell) prior to translation.

Circular RNAs (circRNAs) have been found to occur naturally in cells and have been found to have diverse functions, including both non-coding and protein coding roles in human cells. It has been shown that a circRNA can be engineered by incorporating a self-splicing intron into an RNA molecule (or DNA encoding the RNA molecule) that results in circularization of the RNA, and that an engineered circRNA can have enhanced protein production and stability (Wesselhoeft et al. Nature Communications 2018). In some embodiments, the gene modifying polypeptide is encoded as circRNA. In certain embodiments, the template nucleic acid is a DNA, such as a dsDNA or ssDNA. In certain embodiments, the circDNA comprises a template RNA.

In some embodiments, the circRNA comprises one or more ribozyme sequences. In some embodiments, the ribozyme sequence is activated for autocleavage, e.g., in a host cell, e.g., thereby resulting in linearization of the circRNA. In some embodiments, the ribozyme is activated when the concentration of magnesium reaches a sufficient level for cleavage, e.g., in a host cell. In some embodiments the circRNA is maintained in a low magnesium environment prior to delivery to the host cell. In some embodiments, the ribozyme is a protein-responsive ribozyme. In some embodiments, the ribozyme is a nucleic acid-responsive ribozyme. In some embodiments, the circRNA comprises a cleavage site. In some embodiments, the circRNA comprises a second cleavage site.

In some embodiments, the circRNA is linearized in the nucleus of a target cell. In some embodiments, linearization of a circRNA in the nucleus of a cell involves components present in the nucleus of the cell, e.g., to activate a cleavage event. In some embodiments, a ribozyme, e.g., a ribozyme from a B2 or ALU element, that is responsive to a nuclear element, e.g., a nuclear protein, e.g., a genome-interacting protein, e.g., an epigenetic modifier, e.g., EZH2, is incorporated into a circRNA, e.g., of a gene modifying system. In some embodiments, nuclear localization of the circRNA results in an increase in autocatalytic activity of the ribozyme and linearization of the circRNA.

In some embodiments, the ribozyme is heterologous to one or more of the other components of the gene modifying system. In some embodiments, an inducible ribozyme (e.g., in a circRNA as described herein) is created synthetically, for example, by utilizing a protein ligand-responsive aptamer design. A system for utilizing the satellite RNA of tobacco ringspot virus hammerhead ribozyme with an MS2 coat protein aptamer has been described (Kennedy et al. Nucleic Acids Res 42 (19): 12306-12321 (2014), incorporated herein by reference in its entirety) that results in activation of the ribozyme activity in the presence of the MS2 coat protein. In embodiments, such a system responds to protein ligand localized to the cytoplasm or the nucleus. In some embodiments the protein ligand is not MS2. Methods for generating RNA aptamers to target ligands have been described, for example, based on the systematic evolution of ligands by exponential enrichment (SELEX) (Tuerk and Gold, Science 249 (4968): 505-510 (1990); Ellington and Szostak, Nature 346 (6287): 818-822 (1990); the methods of each of which are incorporated herein by reference) and have, in some instances, been aided by in silico design (Bell et al. PNAS 117 (15): 8486-8493, the methods of which are incorporated herein by reference). Thus, in some embodiments, an aptamer for a target ligand is generated and incorporated into a synthetic ribozyme system, e.g., to trigger ribozyme-mediated cleavage and circRNA linearization, e.g., in the presence of the protein ligand. In some embodiments, circRNA linearization is triggered in the cytoplasm, e.g., using an aptamer that associates with a ligand in the cytoplasm. In some embodiments, circRNA linearization is triggered in the nucleus, e.g., using an aptamer that associates with a ligand in the nucleus. In embodiments, the ligand in the nucleus comprises an epigenetic modifier or a transcription factor. In some embodiments the ligand that triggers linearization is present at higher levels in on-target cells than off-target cells.

It is further contemplated that a nucleic acid-responsive ribozyme system can be employed for circRNA linearization. For example, biosensors that sense defined target nucleic acid molecules to trigger ribozyme activation are described, e.g., in Penchovsky (Biotechnology Advances 32 (5): 1015-1027 (2014), incorporated herein by reference). By these methods, a ribozyme naturally folds into an inactive state and is only activated in the presence of a defined target nucleic acid molecule (e.g., an RNA molecule). In some embodiments, a circRNA of a gene modifying system comprises a nucleic acid-responsive ribozyme that is activated in the presence of a defined target nucleic acid, e.g., an RNA, e.g., an mRNA, miRNA, guide RNA, gRNA, sgRNA, ncRNA, lncRNA, tRNA, snRNA, or mtRNA. In some embodiments the nucleic acid that triggers linearization is present at higher levels in on-target cells than off-target cells.

In some embodiments of any of the aspects herein, a gene modifying system incorporates one or more ribozymes with inducible specificity to a target tissue or target cell of interest, e.g., a ribozyme that is activated by a ligand or nucleic acid present at higher levels in a target tissue or target cell of interest. In some embodiments, the gene modifying system incorporates a ribozyme with inducible specificity to a subcellular compartment, e.g., the nucleus, nucleolus, cytoplasm, or mitochondria. In some embodiments, the ribozyme that is activated by a ligand or nucleic acid present at higher levels in the target subcellular compartment. In some embodiments, an RNA component of a gene modifying system is provided as circRNA, e.g., that is activated by linearization. In some embodiments, linearization of a circRNA encoding a gene modifying polypeptide activates the molecule for translation. In some embodiments, a signal that activates a circRNA component of a gene modifying system is present at higher levels in on-target cells or tissues, e.g., such that the system is specifically activated in these cells.

In some embodiments, an RNA component of a gene modifying system is provided as a circRNA that is inactivated by linearization. In some embodiments, a circRNA encoding the gene modifying polypeptide is inactivated by cleavage and degradation. In some embodiments, a circRNA encoding the gene modifying polypeptide is inactivated by cleavage that separates a translation signal from the coding sequence of the polypeptide. In some embodiments, a signal that inactivates a circRNA component of a gene modifying system is present at higher levels in off-target cells or tissues, such that the system is specifically inactivated in these cells.

### Chemically Modified Nucleic Acids and Nucleic Acid End Features

A nucleic acid described herein (e.g., a template nucleic acid, e.g., a template RNA; or a nucleic acid (e.g., mRNA) encoding a heterologous or retrotransposon gene modifying polypeptide; or a gRNA) can comprise unmodified or modified nucleobases. Naturally occurring RNAs are synthesized from four basic ribonucleotides: ATP, CTP, UTP and GTP, but may contain post-transcriptionally modified nucleotides. Further, approximately one hundred different nucleoside modifications have been identified in RNA (Rozenski, J, Crain, P, and McCloskey, J. (1999). The RNA Modification Database: 1999 update. Nucl Acids Res 27:196-197). An RNA can also comprise wholly synthetic nucleotides that do not occur in nature.

In some embodiments, the chemical modification is one provided in WO/2016/183482, US Pat. Pub. No. 20090286852, of International Application No. WO/2012/019168, WO/2012/045075, WO/2012/135805, WO/2012/158736, WO/2013/039857, WO/2013/039861, WO/2013/052523, WO/2013/090648, WO/2013/096709, WO/2013/101690, WO/2013/106496, WO/2013/130161, WO/2013/151669, WO/2013/151736, WO/2013/151672, WO/2013/151664, WO/2013/151665, WO/2013/151668, WO/2013/151671, WO/2013/151667, WO/2013/151670, WO/2013/151666, WO/2013/151663, WO/2014/028429, WO/2014/081507, WO/2014/093924, WO/2014/093574, WO/2014/113089, WO/2014/144711, WO/2014/144767, WO/2014/144039, WO/2014/152540, WO/2014/152030, WO/2014/152031, WO/2014/152027, WO/2014/152211, WO/2014/158795, WO/2014/159813, WO/2014/164253, WO/2015/006747, WO/2015/034928, WO/2015/034925, WO/2015/038892, WO/2015/048744, WO/2015/051214, WO/2015/051173, WO/2015/051169, WO/2015/058069, WO/2015/085318, WO/2015/089511, WO/2015/105926, WO/2015/164674, WO/2015/196130, WO/2015/196128, WO/2015/196118, WO/2016/011226, WO/2016/011222, WO/2016/011306, WO/2016/014846, WO/2016/022914, WO/2016/036902, WO/2016/077125, or WO/2016/077123, each of which is herein incorporated by reference in its entirety. It is understood that incorporation of a chemically modified nucleotide into a polynucleotide can result in the modification being incorporated into a nucleobase, the backbone, or both, depending on the location of the modification in the nucleotide. In some embodiments, the backbone modification is one provided in EP 2813570, which is herein incorporated by reference in its entirety. In some embodiments, the modified cap is one provided in US Pat. Pub. No. 20050287539, which is herein incorporated by reference in its entirety.

In some embodiments, the chemically modified nucleic acid (e.g., RNA, e.g., mRNA) comprises one or more of ARCA: anti-reverse cap analog (m27.3′-OGP3G), GP3G (Unmethylated Cap Analog), m7GP3G (Monomethylated Cap Analog), m32.2.7GP3G (Trimethylated Cap Analog), m5CTP (5′-methyl-cytidine triphosphate), m6ATP (N6-methyl-adenosine-5′-triphosphate), s2UTP (2-thio-uridine triphosphate), and Y′ (pseudouridine triphosphate).

In some embodiments, the chemically modified nucleic acid comprises a 5′ cap, e.g.: a 7-methylguanosine cap (e.g., a O-Me-m7G cap); a hypermethylated cap analog; an NAD+-derived cap analog (e.g., as described in Kiledjian, Trends in Cell Biology 28, 454-464 (2018)); or a modified, e.g., biotinylated, cap analog (e.g., as described in Bednarek et al., Phil Trans R Soc B 373, 20180167 (2018)).

In some embodiments, the chemically modified nucleic acid comprises a 3′ feature selected from one or more of: a polyA tail; a 16-nucleotide long stem-loop structure flanked by unpaired 5 nucleotides (e.g., as described by Mannironi et al., Nucleic Acid Research 17, 9113-9126 (1989)); a triple-helical structure (e.g., as described by Brown et al., PNAS 109, 19202-19207 (2012)); a tRNA, Y RNA, or vault RNA structure (e.g., as described by Labno et al., Biochemica et Biophysica Acta 1863, 3125-3147 (2016)); incorporation of one or more deoxyribonucleotide triphosphates (dNTPs), 2′O-Methylated NTPs, or phosphorothioate-NTPs; a single nucleotide chemical modification (e.g., oxidation of the 3′ terminal ribose to a reactive aldehyde followed by conjugation of the aldehyde-reactive modified nucleotide); or chemical ligation to another nucleic acid molecule.

In some embodiments, the nucleic acid (e.g., template nucleic acid) comprises one or more modified nucleotides, e.g., selected from dihydrouridine, inosine, 7-methylguanosine, 5-methylcytidine (5mC), 5′ Phosphate ribothymidine, 2′-O-methyl ribothymidine, 2′-O-ethyl ribothymidine, 2′-fluoro ribothymidine, C-5 propynyl-deoxycytidine (pdC), C-5 propynyl-deoxyuridine (pdU), C-5 propynyl-cytidine (pC), C-5 propynyl-uridine (pU), 5-methyl cytidine, 5-methyl uridine, 5-methyl deoxycytidine, 5-methyl deoxyuridine methoxy, 2,6-diaminopurine, 5′-Dimethoxytrityl-N4-ethyl-2′-deoxycytidine, C-5 propynyl-f-cytidine (pfC), C-5 propynyl-f-uridine (pfU), 5-methyl f-cytidine, 5-methyl f-uridine, C-5 propynyl-m-cytidine (pmC), C-5 propynyl-f-uridine (pmU), 5-methyl m-cytidine, 5-methyl m-uridine, LNA (locked nucleic acid), MGB (minor groove binder) pseudouridine (Y), 1-N-methylpseudouridine (1-Me-Ψ), or 5-methoxyuridine (5-MO-U).

In some embodiments, the nucleic acid comprises a backbone modification, e.g., a modification to a sugar or phosphate group in the backbone. In some embodiments, the nucleic acid comprises a nucleobase modification.

In some embodiments, the nucleic acid comprises one or more chemically modified nucleotides of Table 13, one or more chemical backbone modifications of Table 14, one or more chemically modified caps of Table 15. For instance, in some embodiments, the nucleic acid comprises two or more (e.g., 3, 4, 5, 6, 7, 8, 9, or 10 or more) different types of chemical modifications. As an example, the nucleic acid may comprise two or more (e.g., 3, 4, 5, 6, 7, 8, 9, or 10 or more) different types of modified nucleobases, e.g., as described herein, e.g., in Table 13. Alternatively or in combination, the nucleic acid may comprise two or more (e.g., 3, 4, 5, 6, 7, 8, 9, or 10 or more) different types of backbone modifications, e.g., as described herein, e.g., in Table 14. Alternatively or in combination, the nucleic acid may comprise one or more modified cap, e.g., as described herein, e.g., in Table 15. For instance, in some embodiments, the nucleic acid comprises one or more type of modified nucleobase and one or more type of backbone modification; one or more type of modified nucleobase and one or more modified cap; one or more type of modified cap and one or more type of backbone modification; or one or more type of modified nucleobase, one or more type of backbone modification, and one or more type of modified cap.

In some embodiments, the nucleic acid comprises one or more (e.g., 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, 1000, or more) modified nucleobases. In some embodiments, all nucleobases of the nucleic acid are modified. In some embodiments, the nucleic acid is modified at one or more (e.g., 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, 1000, or more) positions in the backbone. In some embodiments, all backbone positions of the nucleic acid are modified.

TABLE 13
Modified nucleotides

| | |
|---|---|
| 5-aza-uridine | N2-methyl-6-thio-guanosine |
| 2-thio-5-aza-midine | N2,N2-dimethyl-6-thio-guanosine |
| 2-thiouridine | pyridin-4-one ribonucleoside |
| 4-thio-pseudouridine | 2-thio-5-aza-uridine |
| 2-thio-pseudouridine | 2-thiomidine |
| 5-hydroxyuridine | 4-thio-pseudomidine |
| 3-methyluridine | 2-thio-pseudowidine |
| 5-carboxymethyl-uridine | 3-methylmidine |
| 1-carboxymethyl-pseudouridine | 1-propynyl-pseudomidine |
| 5-propynyl-uridine | 1-methyl-1-deaza-pseudomidine |
| 1-propynyl-pseudouridine | 2-thio-1-methyl-1-deaza-pseudouridine |
| 5-taurinomethyluridine | 4-methoxy-pseudomidine |
| 1-taurinomethyl-pseudouridine | 5′-O-(1-Thiophosphate)-Adenosine |
| 5-taurinomethyl-2-thio-uridine | 5′-O-(1-Thiophosphate)-Cytidine |
| 1-taurinomethyl-4-thio-uridine | 5′-O-(1-thiophosphate)-Guanosine |
| 5-methyl-uridine | 5′-O-(1-Thiophophate)-Uridine |
| 1-methyl-pseudouridine | 5′-O-(1-Thiophosphate)-Pseudouridine |
| 4-thio-1-methyl-pseudouridine | 2′-O-methyl-Adenosine |
| 2-thio-1-methyl-pseudouridine | 2′-O-methyl-Cytidine |
| 1-methyl-1-deaza-pseudouridine | 2′-O-methyl-Guanosine |
| 2-thio-1-methyl-1-deaza-pseudomidine | 2′-O-methyl-Uridine |
| dihydrouridine | 2′-O-methyl-Pseudouridine |
| dihydropseudouridine | 2′-O-methyl-Inosine |
| 2-thio-dihydromidine | 2-methyladenosine |
| 2-thio-dihydropseudouridine | 2-methylthio-N6-methyladenosine |
| 2-methoxyuridine | 2-methylthio-N6 isopentenyladenosine |
| 2-methoxy-4-thio-uridine | 2-methylthio-N6-(cis- |
| 4-methoxy-pseudouridine | hydroxyisopentenyl)adenosine |
| 4-methoxy-2-thio-pseudouridine | N6-methyl-N6-threonylcarbamoyladenosine |
| 5-aza-cytidine | N6-hydroxynorvalylcarbamoyladenosine |
| pseudoisocytidine | 2-methylthio-N6-hydroxynorvalyl |
| 3-methyl-cytidine | carbamoyladenosine |
| N4-acetylcytidine | 2′-O-ribosyladenosine (phosphate) |
| 5-formylcytidine | 1,2′-O-dimethylinosine |
| N4-methylcytidine | 5,2′-O-dimethylcytidine |
| 5-hydroxymethylcytidine | N4-acetyl-2′-O-methylcytidine |
| 1-methyl-pseudoisocytidine | Lysidine |
| pyrrolo-cytidine | 7-methylguanosine |
| pyrrolo-pseudoisocytidine | N2,2′-O-dimethylguanosine |
| 2-thio-cytidine | N2,N2,2′-O-trimethylguanosine |
| 2-thio-5-methyl-cytidine | 2′-O-ribosylguanosine (phosphate) |
| 4-thio-pseudoisocytidine | Wybutosine |
| 4-thio-1-methyl-pseudoisocytidine | Peroxywybutosine |
| 4-thio-1-methyl-1-deaza-pseudoisocytidine | Hydroxywybutosine |
| 1-methyl-1-deaza-pseudoisocytidine | undermodified hydroxywybutosine |
| zebularine | methylwyosine |
| 5-aza-zebularine | queuosine |
| 5-methyl-zebularine | epoxyqueuosine |
| 5-aza-2-thio-zebularine | galactosyl-queuosine |
| 2-thio-zebularine | mannosyl-queuosine |
| 2-methoxy-cytidine | 7-cyano-7-deazaguanosine |
| 2-methoxy-5-methyl-cytidine | 7-aminomethyl-7-deazaguanosine |
| 4-methoxy-pseudoisocytidine | archaeosine |
| 4-methoxy-1-methyl-pseudoisocytidine | 5,2′-O-dimethyluridine |
| 2-aminopurine | 4-thiouridine |
| 2,6-diaminopurine | 5-methyl-2-thiouridine |
| 7-deaza-adenine | 2-thio-2′-O-methyluridine |
| 7-deaza-8-aza-adenine | 3-(3-amino-3-carboxypropyl)uridine |
| 7-deaza-2-aminopurine | 5-methoxyuridine |
| 7-deaza-8-aza-2-aminopurine | uridine 5-oxyacetic acid |
| 7-deaza-2,6- diaminopurine | uridine 5-oxyacetic acid methyl ester |
| 7-deaza-8-aza-2,6-diarninopurine | 5-(carboxyhydroxymethyl)uridine) |
| 1-methyladenosine | 5-(carboxyhydroxymethyl)uridine methyl ester |
| N6-isopentenyladenosine | 5-methoxycarbonylmethyluridine |
| N6-(cis-hydroxyisopentenyl)adenosine | 5-methoxycarbonylmethyl-2′-O-methyluridine |
| 2-methylthio-N6-(cis-hydroxyisopentenyl) | 5-methoxycarbonylmethyl-2-thiouridine |
| adenosine | 5-aminomethyl-2-thiouridine |
| N6-glycinylcarbamoyladenosine | 5-methylaminomethyluridine |
| N6-threonylcarbamoyladenosine | 5-methylaminomethyl-2-thiouridine |
| 2-methylthio-N6-threonyl | 5-methylaminomethyl-2-selenouridine |
| carbamoyladenosine | 5-carbamoylmethyluridine |
| N6,N6-dimethyladenosine | 5-carbamoylmethyl-2′-O-methyluridine |
| 7-methyladenine | 5-carboxymethylaminomethyluridine |
| 2-methylthio-adenine | 5-carboxymethylaminomethyl-2′-O- |
| 2-methoxy-adenine | methyluridine |
| inosine | 5-carboxymethylaminomethyl-2-thiouridine |
| 1-methyl-inosine | N4,2′-O-dimethylcytidine |
| wyosine | 5-carboxymethyluridine |
| wybutosine | N6,2′-O-dimethyladenosine |
| 7-deaza-guanosine | N,N6,O-2′-trimethyladenosine |
| 7-deaza-8-aza-guanosine | N2,7-dimethylguanosine |
| 6-thio-guanosine | N2,N2,7-trimethylguanosine |
| 6-thio-7-deaza-guanosine | 3,2′-O-dimethyluridine |
| 6-thio-7-deaza-8-aza-guanosine | 5-methyldihydrouridine |
| 7-methyl-guanosine | 5-formyl-2′-O-methylcytidine |
| 6-thio-7-methyl-guanosine | 1,2′-O-dimethylguanosine |
| 7-methylinosine | 4-demethylwyosine |
| 6-methoxy-guanosine | Isowyosine |
| 1-methylguanosine | N6-acetyladenosine |
| N2-methylguanosine | |
| N2,N2-dimethylguanosine | |
| 8-oxo-guanosine | |
| 7-methyl-8-oxo-guanosine | |
| 1-methyl-6-thio-guanosine | |

TABLE 14
Backbone modifications

2′-O-Methyl backbone
Peptide Nucleic Acid (PNA) backbone
phosphorothioate backbone
morpholino backbone
carbamate backbone
siloxane backbone
sulfide backbone
sulfoxide backbone
sulfone backbone
formacetyl backbone
thioformacetyl backbone
methyleneformacetyl backbone
riboacetyl backbone
alkene containing backbone
sulfamate backbone
sulfonate backbone
sulfonamide backbone
methyleneimino backbone
methylenehydrazino backbone
amide backbone

TABLE 15
Modified caps

m7GpppA
m7GpppC
m2,7GpppG
m2,2,7GpppG
m7Gpppm7G
m7,2'OmeGpppG
m72'dGpppG
m7,3'OmeGpppG
m7,3'dGpppG
GppppG
m7GppppG
m7GppppA
m7GppppC
m2,7GppppG
m2,2,7GppppG
m7Gppppm7G
m7,2'OmeGppppG
m72'dGppppG
m7,3'OmeGppppG
m7,3'dGppppG

The nucleotides comprising the template of the heterologous or retrotransposon gene modifying system can be natural or modified bases, or a combination thereof. For example, the template may contain pseudouridine, dihydrouridine, inosine, 7-methylguanosine, or other modified bases. In some embodiments, the template may contain locked nucleic acid nucleotides. In some embodiments, the modified bases used in the template do not inhibit the reverse transcription of the template. In some embodiments, the modified bases used in the template may improve reverse transcription, e.g., specificity or fidelity.

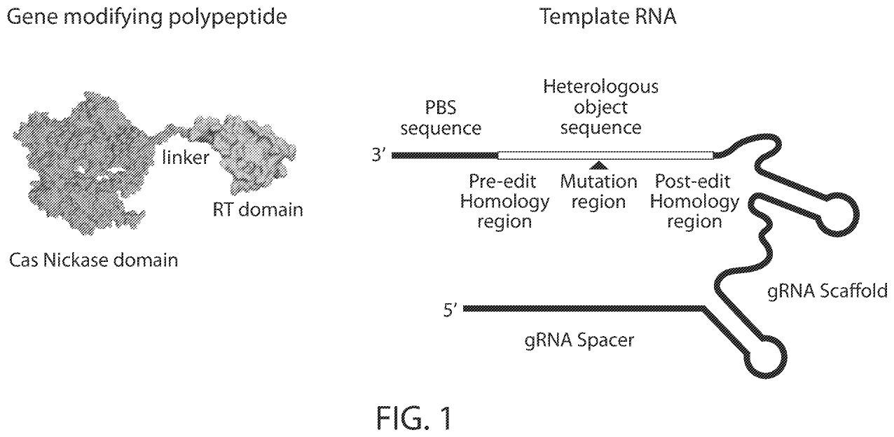

In some embodiments, an RNA component of the system (e.g., a template RNA or a gRNA) comprises one or more nucleotide modifications. In some embodiments, the modification pattern of a gRNA can significantly affect in vivo activity compared to unmodified or end-modified guides (e.g., as shown in FIG. 1D from Finn et al. Cell Rep 22 (9): 2227-2235 (2018); incorporated herein by reference in its entirety). Without wishing to be bound by theory, this process may be due, at least in part, to a stabilization of the RNA conferred by the modifications. Non-limiting examples of such modifications may include 2′-O-methyl(2′-O-Me), 2′-0-(2-methoxyethyl) (2′-0-MOE), 2′-fluoro (2′-F), phosphorothioate (PS) bond between nucleotides, G-C substitutions, and inverted abasic linkages between nucleotides and equivalents thereof.

In some embodiments, the template RNA (e.g., at the portion thereof that binds a target site) or the guide RNA comprises a 5′ terminus region. In some embodiments, the template RNA or the guide RNA does not comprise a 5′ terminus region. In some embodiments, the 5′ terminus region comprises a gRNA spacer region, e.g., as described with respect to sgRNA in Briner A E et al, Molecular Cell 56:333-339 (2014) (incorporated herein by reference in its entirety; applicable herein, e.g., to all guide RNAs). In some embodiments, the 5′ terminus region comprises a 5′ end modification. In some embodiments, a 5′ terminus region with or without a spacer region may be associated with a crRNA, trRNA, sgRNA and/or dgRNA. The gRNA spacer region can, in some instances, comprise a guide region, guide domain, or targeting domain.

In some embodiments, the template RNAs (e.g., at the portion thereof that binds a target site) or guide RNAs described herein comprises any of the sequences shown in Table 4 of WO2018107028A1, incorporated herein by reference in its entirety. In some embodiments, where a sequence shows a guide and/or spacer region, the composition may comprise this region or not. In some embodiments, a guide RNA comprises one or more of the modifications of any of the sequences shown in Table 4 of WO2018107028A1, e.g., as identified therein by a SEQ ID NO. In embodiments, the nucleotides may be the same or different, and/or the modification pattern shown may be the same or similar to a modification pattern of a guide sequence as shown in Table 4 of WO2018107028A1. In some embodiments, a modification pattern includes the relative position and identity of modifications of the gRNA or a region of the gRNA (e.g. 5′ terminus region, lower stem region, bulge region, upper stem region, nexus region, hairpin 1 region, hairpin 2 region, 3′ terminus region). In some embodiments, the modification pattern contains at least 50%, 55%, 60%, 70%, 75%, 80%, 85%, 90%, 95%, 96%, 97%, 98%, 99%, or 100% of the modifications of any one of the sequences shown in the sequence column of Table 4 of WO2018107028A1, and/or over one or more regions of the sequence. In some embodiments, the modification pattern is at least 50%, 55%, 60%, 70%, 75%, 80%, 85%, 90%, 95%, 96%, 97%, 98%, 99%, or 100% identical to the modification pattern of any one of the sequences shown in the sequence column of Table 4 of WO2018107028A1. In some embodiments, the modification pattern is at least 50%, 55%, 60%, 70%, 75%, 80%, 85%, 90%, 95%, 96%, 97%, 98%, 99%, or 100% identical over one or more regions of the sequence shown in Table 4 of WO2018107028A1, e.g., in a 5 ‘terminus region, lower stem region, bulge region, upper stem region, nexus region, hairpin 1 region, hairpin 2 region, and/or 3’ terminus region. In some embodiments, the modification pattern is least 50%, 55%, 60%, 70%, 75%, 80%, 85%, 90%, 95%, 96%, 97%, 98%, 99%, or 100% identical to the modification pattern of a sequence over the 5′ terminus region. In some embodiments, the modification pattern is least 50%, 55%, 60%, 70%, 75%, 80%, 85%, 90%, 95%, 96%, 97%, 98%, 99%, or 100% identical over the lower stem. In some embodiments, the modification pattern is least 50%, 55%, 60%, 70%, 75%, 80%, 85%, 90%, 95%, 96%, 97%, 98%, 99%, or 100% identical over the bulge. In some embodiments, the modification pattern is least 50%, 55%, 60%, 70%, 75%, 80%, 85%, 90%, 95%, 96%, 97%, 98%, 99%, or 100% identical over the upper stem. In some embodiments, the modification pattern is least 50%, 55%, 60%, 70%, 75%, 80%, 85%, 90%, 95%, 96%, 97%, 98%, 99%, or 100% identical over the nexus. In some embodiments, the modification pattern is least 50%, 55%, 60%, 70%, 75%, 80%, 85%, 90%, 95%, 96%, 97%, 98%, 99%, or 100% identical over the hairpin 1. In some embodiments, the modification pattern is least 50%, 55%, 60%, 70%, 75%, 80%, 85%, 90%, 95%, 96%, 97%, 98%, 99%, or 100% identical over the hairpin 2. In some embodiments, the modification pattern is least 50%, 55%, 60%, 70%, 75%, 80%, 85%, 90%, 95%, 96%, 97%, 98%, 99%, or 100% identical over the 3′ terminus. In some embodiments, the modification pattern differs from the modification pattern of a sequence of Table 4 of WO2018107028A1, or a region (e.g. 5′ terminus, lower stem, bulge, upper stem, nexus, hairpin 1, hairpin 2, 3′ terminus) of such a sequence, e.g., at 0, 1, 2, 3, 4, 5, 6, or more nucleotides. In some embodiments, the gRNA comprises modifications that differ from the modifications of a sequence of Table 4 of WO2018107028A1, e.g., at 0, 1, 2, 3, 4, 5, 6, or more nucleotides. In some embodiments, the gRNA comprises modifications that differ from modifications of a region (e.g. 5 ‘terminus, lower stem, bulge, upper stem, nexus, hairpin 1, hairpin 2, 3’ terminus) of a sequence of Table 4 of WO2018107028A1, e.g., at 0, 1, 2, 3, 4, 5, 6, or more nucleotides.

In some embodiments, the template RNAs (e.g., at the portion thereof that binds a target site) or the gRNA comprises a 2′-O-methyl(2′-O-Me) modified nucleotide. In some embodiments, the gRNA comprises a 2′-O-(2-methoxy ethyl) (2′-O-moe) modified nucleotide. In some embodiments, the gRNA comprises a 2′-fluoro (2′-F) modified nucleotide. In some embodiments, the gRNA comprises a phosphorothioate (PS) bond between nucleotides. In some embodiments, the gRNA comprises a 5′ end modification, a 3′ end modification, or 5′ and 3′ end modifications. In some embodiments, the 5′ end modification comprises a phosphorothioate (PS) bond between nucleotides. In some embodiments, the 5′ end modification comprises a 2′-O-methyl(2′-O-Me), 2′-O-(2-methoxy ethyl) (2′-O-MOE), and/or 2′-fluoro (2′-F) modified nucleotide. In some embodiments, the 5′ end modification comprises at least one phosphorothioate (PS) bond and one or more of a 2′-O-methyl(2′-O-Me), 2′-O-(2-methoxyethyl) (2′-O-MOE), and/or 2′-fluoro (2′-F) modified nucleotide. The end modification may comprise a phosphorothioate (PS), 2′-O-methyl(2′-O-Me), 2′-O-(2-methoxyethyl) (2′-O-MOE), and/or 2′-fluoro (2′-F) modification. Equivalent end modifications are also encompassed by embodiments described herein. In some embodiments, the template RNA or gRNA comprises an end modification in combination with a modification of one or more regions of the template RNA or gRNA. Additional exemplary modifications and methods for protecting RNA, e.g., gRNA, and formulae thereof, are described in WO2018126176A1, which is incorporated herein by reference in its entirety.

In some embodiments, a template RNA described herein comprises three phosphorothioate linkages at the 5′ end and three phosphorothioate linkages at the 3′ end. In some embodiments, a template RNA described herein comprises three 2′-O-methyl ribonucleotides at the 5′ end and three 2′-O-methyl ribonucleotides at the 3′ end. In some embodiments, the 5′ most three nucleotides of the template RNA are 2′-O-methyl ribonucleotides, the 5′ most three internucleotide linkages of the template RNA are phosphorothioate linkages, the 3′ most three nucleotides of the template RNA are 2′-O-methyl ribonucleotides, and the 3′ most three internucleotide linkages of the template RNA are phosphorothioate linkages. In some embodiments, the template RNA comprises alternating blocks of ribonucleotides and 2′-O-methyl ribonucleotides, for instance, blocks of between 12 and 28 nucleotides in length. In some embodiments, the central portion of the template RNA comprises the alternating blocks and the 5′ and 3′ ends each comprise three 2′-O-methyl ribonucleotides and three phosphorothioate linkages.

In some embodiments, structure-guided and systematic approaches are used to introduce modifications (e.g., 2′-OMe-RNA, 2′-F-RNA, and PS modifications) to a template RNA or guide RNA, for example, as described in Mir et al. Nat Commun 9:2641 (2018) (incorporated by reference herein in its entirety). In some embodiments, the incorporation of 2′-F-RNAs increases thermal and nuclease stability of RNA:RNA or RNA:DNA duplexes, e.g., while minimally interfering with C3′-endo sugar puckering. In some embodiments, 2′-F may be better tolerated than 2′-OMe at positions where the 2′-OH is important for RNA:DNA duplex stability. In some embodiments, a crRNA comprises one or more modifications that do not reduce Cas9 activity, e.g., C10, C20, or C21 (fully modified), e.g., as described in Supplementary Table 1 of Mir et al. Nat Commun 9:2641 (2018), incorporated herein by reference in its entirety. In some embodiments, a tracrRNA comprises one or more modifications that do not reduce Cas9 activity, e.g., T2, T6, T7, or T8 (fully modified) of Supplementary Table 1 of Mir et al. Nat Commun 9:2641 (2018). In some embodiments, a crRNA comprises one or more modifications (e.g., as described herein) may be paired with a tracrRNA comprising one or more modifications, e.g., C20 and T2. In some embodiments, a gRNA comprises a chimera, e.g., of a crRNA and a tracrRNA (e.g., Jinek et al. Science 337 (6096): 816-821 (2012)). In embodiments, modifications from the crRNA and tracrRNA are mapped onto the single-guide chimera, e.g., to produce a modified gRNA with enhanced stability.

In some embodiments, gRNA molecules may be modified by the addition or subtraction of the naturally occurring structural components, e.g., hairpins. In some embodiments, a gRNA may comprise a gRNA with one or more 3′ hairpin elements deleted, e.g., as described in WO2018106727, incorporated herein by reference in its entirety. In some embodiments, a gRNA may contain an added hairpin structure, e.g., an added hairpin structure in the spacer region, which was shown to increase specificity of a CRISPR-Cas system in the teachings of Kocak et al. Nat Biotechnol 37 (6): 657-666 (2019). Additional modifications, including examples of shortened gRNA and specific modifications improving in vivo activity, can be found in US20190316121, incorporated herein by reference in its entirety.

In some embodiments, structure-guided and systematic approaches (e.g., as described in Mir et al. Nat Commun 9:2641 (2018); incorporated herein by reference in its entirety) are employed to find modifications for the template RNA. In embodiments, the modifications are identified with the inclusion or exclusion of a guide region of the template RNA. In some embodiments, a structure of polypeptide bound to template RNA is used to determine non-protein-contacted nucleotides of the RNA that may then be selected for modifications, e.g., with lower risk of disrupting the association of the RNA with the polypeptide. Secondary structures in a template RNA can also be predicted in silico by software tools, e.g., the RNAstructure tool available at rna.urmc.rochester.edu/RNAstructureWeb (Bellaousov et al. Nucleic Acids Res 41: W471-W474 (2013); incorporated by reference herein in its entirety), e.g., to determine secondary structures for selecting modifications, e.g., hairpins, stems, and/or bulges.

### Production of Compositions and Systems

As will be appreciated by one of skill, methods of designing and constructing nucleic acid constructs and proteins or polypeptides (such as the systems, constructs and polypeptides described herein) are routine in the art. Generally, recombinant methods may be used. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013). Methods of designing, preparing, evaluating, purifying and manipulating nucleic acid compositions are described in Green and Sambrook (Eds.), Molecular Cloning: A Laboratory Manual (Fourth Edition), Cold Spring Harbor Laboratory Press (2012).

The disclosure provides, in part, a nucleic acid, e.g., vector, encoding a heterologous or retrotransposon gene modifying polypeptide described herein, a template nucleic acid described herein, or both. In some embodiments, a vector comprises a selective marker, e.g., an antibiotic resistance marker. In some embodiments, the antibiotic resistance marker is a kanamycin resistance marker. In some embodiments, the antibiotic resistance marker does not confer resistance to beta-lactam antibiotics. In some embodiments, the vector does not comprise an ampicillin resistance marker. In some embodiments, the vector comprises a kanamycin resistance marker and does not comprise an ampicillin resistance marker. In some embodiments, a vector encoding a gene modifying polypeptide is integrated into a target cell genome (e.g., upon administration to a target cell, tissue, organ, or subject). In some embodiments, a vector encoding a gene modifying polypeptide is not integrated into a target cell genome (e.g., upon administration to a target cell, tissue, organ, or subject). In some embodiments, a vector encoding a template nucleic acid (e.g., template RNA) is not integrated into a target cell genome (e.g., upon administration to a target cell, tissue, organ, or subject). In some embodiments, if a vector is integrated into a target site in a target cell genome, the selective marker is not integrated into the genome. In some embodiments, if a vector is integrated into a target site in a target cell genome, genes or sequences involved in vector maintenance (e.g., plasmid maintenance genes) are not integrated into the genome. In some embodiments, if a vector is integrated into a target site in a target cell genome, transfer regulating sequences (e.g., inverted terminal repeats, e.g., from an AAV) are not integrated into the genome. In some embodiments, administration of a vector (e.g., encoding a gene modifying polypeptide described herein, a template nucleic acid described herein, or both) to a target cell, tissue, organ, or subject results in integration of a portion of the vector into one or more target sites in the genome(s) of said target cell, tissue, organ, or subject. In some embodiments, less than 99, 95, 90, 80, 70, 60, 50, 40, 30, 20, 10, 5, 4, 3, 2, or 1% of target sites (e.g., no target sites) comprising integrated material comprise a selective marker (e.g., an antibiotic resistance gene), a transfer regulating sequence (e.g., an inverted terminal repeat, e.g., from an AAV), or both from the vector.

Exemplary methods for producing a therapeutic pharmaceutical protein or polypeptide described herein involve expression in mammalian cells, although recombinant proteins can also be produced using insect cells, yeast, bacteria, or other cells under control of appropriate promoters. Mammalian expression vectors may comprise non-transcribed elements such as an origin of replication, a suitable promoter, and other 5′ or 3′ flanking non-transcribed sequences, and 5′ or 3′ non-translated sequences such as necessary ribosome binding sites, a polyadenylation site, splice donor and acceptor sites, and termination sequences. DNA sequences derived from the SV40 viral genome, for example, SV40 origin, early promoter, splice, and polyadenylation sites may be used to provide other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

Various mammalian cell culture systems can be employed to express and manufacture recombinant protein. Examples of mammalian expression systems include CHO, COS, HEK293, HeLA, and BHK cell lines. Processes of host cell culture for production of protein therapeutics are described in Zhou and Kantardjieff (Eds.), Mammalian Cell Cultures for Biologics Manufacturing (Advances in Biochemical Engineering/Biotechnology), Springer (2014). Compositions described herein may include a vector, such as a viral vector, e.g., a lentiviral vector, encoding a recombinant protein. In some embodiments, a vector, e.g., a viral vector, may comprise a nucleic acid encoding a recombinant protein.

Purification of protein therapeutics is described in Franks, Protein Biotechnology: Isolation, Characterization, and Stabilization, Humana Press (2013); and in Cutler, Protein Purification Protocols (Methods in Molecular Biology), Humana Press (2010).

The disclosure also provides compositions and methods for the production of template nucleic acid molecules (e.g., template RNAs) with specificity for a gene modifying polypeptide and/or a genomic target site. In an aspect, the method comprises production of RNA segments including an upstream homology segment, a heterologous object sequence segment, a gene modifying polypeptide binding motif, and a gRNA segment.

### Therapeutic Applications

In some embodiments, a heterologous or retrotransposon gene modifying system as described herein can be used to modify a cell (e.g., an animal cell, plant cell, or fungal cell). In some embodiments, a gene modifying system as described herein can be used to modify a mammalian cell (e.g., a human cell). In some embodiments, a gene modifying system as described herein can be used to modify a cell from a livestock animal (e.g., a cow, horse, sheep, goat, pig, llama, alpaca, camel, yak, chicken, duck, goose, or ostrich). In some embodiments, a gene modifying system as described herein can be used as a laboratory tool or a research tool, or used in a laboratory method or research method, e.g., to modify an animal cell, e.g., a mammalian cell (e.g., a human cell), a plant cell, or a fungal cell.

By integrating coding genes into a RNA sequence template, the gene modifying system can address therapeutic needs, for example, by providing expression of a therapeutic transgene in individuals with loss-of-function mutations, by replacing gain-of-function mutations with normal transgenes, by providing regulatory sequences to eliminate gain-of-function mutation expression, and/or by controlling the expression of operably linked genes, transgenes and systems thereof. In certain embodiments, the RNA sequence template encodes a promotor region specific to the therapeutic needs of the host cell, for example a tissue specific promotor or enhancer. In still other embodiments, a promotor can be operably linked to a coding sequence.

In some embodiments, insertion of the system into a target cell results in a mutation in that causes disruption of the TRAC gene. In some embodiments, the disruption comprises a frameshift mutation within the TRAC gene. In some embodiments, the disruption comprises generation of a stop codon (e.g., a premature stop codon) into the TRAC gene. In some embodiments, the disruption results in a knock out of the TRAC gene. In some embodiments, the disruption comprises a deletion, e.g., a deletion of a portion of the TRAC gene.

In some embodiments, insertion of the system into a target cell results in a mutation in that disrupts splicing of the TRAC gene. In some embodiments, installation of the mutation results in a loss of TRAC expression. In some embodiments, insertion of the system into a target cell results in a mutation in that causes disruption of the B2M gene. In some embodiments, the disruption comprises a frameshift mutation within the B2M gene. In some embodiments, the disruption comprises generation of a stop codon (e.g., a premature stop codon) into the B2M gene. In some embodiments, the disruption results in a knock out of the B2M gene. In some embodiments, the disruption comprises a deletion, e.g., a deletion of a portion of the B2M gene.

In some embodiments, insertion of the system into a target cell results in a mutation in that disrupts splicing of the B2M gene. In some embodiments, installation of the mutation results in a loss of B2M expression.

In some embodiments, insertion of the system into a target cell results in a mutation in that causes disruption of the TRAC gene and B2M gene. In some embodiments, the disruption comprises a frameshift mutation within the TRAC gene and B2M gene. In some embodiments, the disruption comprises generation of a stop codon (e.g., a premature stop codon) into the TRAC gene and B2M gene. In some embodiments, the disruption results in a knock out of the TRAC gene and B2M gene. In some embodiments, the disruption comprises a deletion, e.g., a deletion of a portion of the TRAC gene and B2M gene.

In some embodiments, insertion of the system into a target cell results in a mutation in that disrupts splicing of the TRAC gene and B2M gene. In some embodiments, installation of the mutation results in a loss of TRAC expression and loss of B2M expression.

In some embodiments, treatment with a system disclosed herein results in disruption of the TRAC gene in between about 15% to about 52% of cells. In some embodiments, treatment with a system disclosed herein results in disruption of the TRAC gene in between about 15% to about 52% of DNA isolated from the treated cells.

In some embodiments, treatment with a system disclosed herein results in disruption of the B2M gene in between about 15% to about 75%) of cells. In some embodiments, treatment with a system disclosed herein results in disruption of the B2M gene in between about 15% to about 75%) of DNA isolated from the treated cells.

In some embodiments, treatment with a system disclosed herein results in disruption of the TRAC gene and the B2M gene in between about 45% to about 75% of cells. In some embodiments, treatment with a system disclosed herein results in disruption of the TRAC gene and the B2M gene in between about 45% to about 75% of DNA isolated from the treated cells.

In some embodiments, treatment with a system disclosed herein does not result in a translocation event.

### Applications Including Retrotransposon Gene Modifying Systems

By integrating coding genes into a RNA sequence template, the retrotransposon gene modifying system can address therapeutic needs, for example, by providing expression of a therapeutic transgene in individuals with loss-of-function mutations, by replacing gain-of-function mutations with normal transgenes, by providing regulatory sequences to eliminate gain-of-function mutation expression, and/or by controlling the expression of operably linked genes, transgenes and systems thereof. In certain embodiments, the RNA sequence template encodes a promotor region specific to the therapeutic needs of the host cell, for example a tissue specific promotor or enhancer. In still other embodiments, a promotor can be operably linked to a coding sequence.

In some embodiments, the heterologous object sequence encodes a membrane protein, e.g., a CAR or a membrane protein other than a CAR, and/or an endogenous human membrane protein. In some embodiments, the heterologous object sequence encodes an extracellular protein. In some embodiments, the heterologous object sequence encodes an enzyme, a structural protein, a signaling protein, a regulatory protein, a transport protein, a sensory protein, a motor protein, a defense protein, or a storage protein. Other exemplary proteins that may be encoded by a heterologous object sequence include, without limitation, an immune receptor protein, e.g. a synthetic immune receptor protein such as a chimeric antigen receptor protein (CAR), a T cell receptor, a B cell receptor, or an antibody.

### Chimeric Antigen Receptors

In some embodiments, the heterologous object sequence encodes a chimeric antigen receptor (CAR) comprising an antigen binding domain. In some embodiments, the CAR is or comprises a first generation CAR comprising an antigen binding domain, a transmembrane domain, and a single intracellular signaling domain. In some embodiments, the CAR is or comprises a second generation CAR comprising an antigen binding domain, a transmembrane domain, and two intracellular signaling domains (e.g., a first intracellular signaling domain and a second intracellular signaling domain). In some embodiments, the CAR comprises a third generation CAR comprising an antigen binding domain, a transmembrane domain, and at least three intracellular signaling domains. In some embodiments, a fourth generation CAR comprising an antigen binding domain, a transmembrane domain, three or four intracellular signaling domains, and a domain which upon successful signaling of the CAR induces expression of a cytokine gene. In some embodiments, the antigen binding domain is or comprises an scFv or Fab.

### Antigen Binding Domains

In some embodiments, a CAR antigen binding domain is or comprises an antibody or antigen-binding portion thereof. In some embodiments, a CAR antigen binding domain is or comprises an scFv or Fab. In some embodiments, an antigen binding domain binds to a cell surface antigen of a cell. In some embodiments, a cell surface antigen is characteristic of one type of cell. In some embodiments, a cell surface antigen is characteristic of more than one type of cell.

In some embodiments, the antigen binding domain targets an antigen characteristic of a neoplastic cell. In some embodiments, the antigen characteristic of a neoplastic cell is selected from a receptor listed in Table C1, or an antigenic fragment or antigenic portion thereof. In some embodiments, the antigen binding domain binds one or more antigens of a blood cancer (e.g., a leukemia or a lymphoma). In some embodiments, the blood cancer antigen is a B cell antigen. In some embodiments, the antigen binding domain binds an antigen of a solid tumor.

TABLE C1
Exemplary Neoplastic Cell Antigens

| | | | | |
|---|---|---|---|---|
| cell surface | NAV1.6 | Carbonic | prostate | p53 mutant |
| receptor | | anhydrase IX | specific | |
| | | | membrane | |
| | | | antigen | |
| | | | (PSMA) | |
| an ion | NAV1.7 | PSMA | ROR1 | prostein |
| channel- | | | | |
| linked | | | | |
| receptor | | | | |
| an enzyme- | NAV1.8 | Folate | FLT3 | survivin |
| linked | | binding | | |
| receptor | | protein | | |
| a G protein- | NAV1.9 | Gangliosides | FAP | telomerase |
| coupled | | | | |
| receptor | | | | |
| receptor | sphingosin-1- | CD2 | TAG72 | PCTA- |
| tyrosine | phosphate | | | 1/Galectin 8 |
| kinase | receptor | | | |
| | (S1P1R) | | | |
| tyrosine | NMDA | CD3 | CD38 | MelanA/MART1 |
| kinase | channel | | | |
| associated | | | | |
| receptor | | | | |
| receptor-like | transmembrane | GM2) | CD44v6 | Ras mutant |
| tyrosine | protein | | | |
| phosphatase | | | | |
| receptor | multispan | Lewis-γ² | CEA | hTERT |
| serine/ | transmembrane | | | |
| threonine | protein | | | |
| kinase | | | | |
| receptor | T-cell receptor | VEGF | EPCAM | sarcoma |
| guanylyl | motifs | | | translocation |
| cyclase | | | | breakpoints |
| histidine | T-cell alpha | VEGFR | B7H3 | ML-IAP |
| kinase | chains | 1/2/3 | | |
| associated | | | | |
| receptor | | | | |
| ErbB1/EGFR | T-cell β chains | αVβ3 | KIT | ERG |
| | | | | (TMPRSS2 ETS |
| | | | | fusion gene) |
| ErbB2/HER2 | T-cell γ chains | α5β1 | interleukin-11 | NA17 |
| | | | receptor a (IL- | |
| | | | 11Ra) | |
| ErbB3/HER3 | T-cell δ chains | ErbB1/EGFR | PSCA | PAX3 |
| and | CCR7 | ErbB1/HER2 | PRSS21 | Androgen |
| ErbB4/HER4) | | | | receptor |
| FGF1 | CD3 | ErB3 | VEGFR2 | Cyclin B1 |
| FGF2 | CD4 | c-MET | LewisY | MYCN |
| FGF3 | CD5 | IGF1R | CD24 | RhoC |
| FGF4 | CD7 | EphA3 | platelet- | TRP-2 |
| | | | derived | |
| | | | growth factor | |
| | | | receptor-beta | |
| | | | (PDGFR-beta) | |
| FGF5 | CD8 | TRAIL-R1 | SSEA-4 | CYPIB I |
| FGF6 | CD11b | TRAIL-R2 | CD20 | BORIS |
| FGF7 | CD11c | RANKL | MUC1 | SART3 |
| FGF18 | CD16 | FAP | NCAM | PAX5 |
| FGF21 | CD19 | Tenascin | Prostase | OY-TES1 |
| VEGF-A | CD20 | PDL-1 | PAP | LCK |
| VEGF-B | CD21 | BAFF | ELF2M | AKAP-4 |
| VEGF-C | CD22 | HDAC | Ephrin B2 | SSX2 |
| VEGF-D | CD25 | ABL | IGF-1 | RAGE-1 |
| | | | receptor | |
| PIGF | CD28 | FLT3 | CAIX | human |
| | | | | telomerase |
| | | | | reverse |
| | | | | transcriptase |
| RET | CD34 | KIT | LMP2 | RU1 |
| Receptor and | | | | |
| the Eph | | | | |
| Receptor | | | | |
| Family | | | | |
| including | | | | |
| EphAl | | | | |
| EphA2 | CD35 | MET | gplOO | RU2 |
| EphA3 | CD40 | RET | bcr-abl | intestinal |
| | | | | carboxyl esterase |
| EphA4 | CD45RA | IL-1β | tyrosinase | mut hsp70-2 |
| EphA5 | CD45RO | ALK | Fucosyl GM1 | CD79a |
| EphA6 | CD52 | RANKL | sLe | CD79b |
| EphA7 | CD56 | mTOR | GM3 | CD72 |
| EphA8 | CD62L | CTLA-4 | TGS5 | LAIR1 |
| EphA9 | CD68 | IL-6 | HMWMAA | FCAR |
| EphA10 | CD80 | IL-6R | o-acetyl-GD2 | LILRA2 |
| EphB1 | CD95 | JAK3 | Folate | CD300LF |
| | | | receptor beta | |
| EphB2. | CD117 | BRAF | TEM1/CD248 | CLEC12A |
| EphB3 | | | | |
| EphB4 | CD127 | PTCH | TEM7R | BST2 |
| EphB6 | CD133 | PIGF | CLDN6 | EMR2 |
| CXCR1 | CD137 (4-1 | ANPEP | GPRC5D | LY75 |
| | BB) | | | |
| CXCR2 | CD163 | TIMP 1 | CXORF61 | GPC3 |
| CXCR3 | F4/80 | PLAUR | CD97 | FCRL5 |
| CXCR4 | IL-4Ra | PTPRJ | CD179a | IGLL 1 |
| CXCR6 | Sca-1 | LTBR | ALK | a neoantigen |
| CCR1 | CTLA-4 | ANTXR1 | Polysialic | CD133 |
| | | | acid | |
| CCR2 | GITR | Folate | PLACI | CD15 |
| | | receptor alpha | | |
| | | (FRa) | | |
| CCR3 | GARP | ERBB2 | GloboH | CD184 |
| | | (Her2/neu) | | |
| CCR4 | LAP | EphA2 | NY-BR-1 | CD24 |
| CCR5 | granzyme B | IL-13Ra2 | UPK2 | CD56 |
| CCR6 | LFA-1 | epidermal | HAVCR1 | CD26 |
| | | growth factor | | |
| | | receptor | | |
| | | (EGFR) | | |
| CCR8 | transferrin | Mesothelin | ADRB3 | CD29 |
| | receptor | | | |
| CFTR | NKp46 | TSHR | PANX3 | CD44 |
| CIC-1 | perforin | CD19 | GPR20 | HLA-A |
| CIC-2 | CD4+ | CD123 | LY6K | HLA-B |
| CIC-4 | Th1 | CD22 | OR51E2 | HLA-C |
| CIC-5 | Th2 | CD30 | TARP | (HLA-A |
| CIC-7 | Th17 | CD171 | WT1 | B |
| CIC-Ka | Th40 | CS-1 | NY-ESO-1 | C) CD49f |
| CIC-Kb | Th22 | CLL-1 | LAGE-la | CD151 CD340 |
| Bestrophins | Th9 | CD33 | MAGE-A1 | CD200 |
| TMEM16A | Tfh | EGFRvIII | legumain | tkrA |
| GABA | Canonical | GD2 | HPV E6 | trkB |
| receptor | Treg. FoxP3+ | | | |
| glycin | Tr1 | GD3 | E7 | or trkC |
| receptor | | | | |
| ABC | Th3 | BCMA | ETV6-AML | TAG-72 |
| transporters | | | | |
| NAV1.1 | Treg17 | MUC16 | sperm protein | Tn Ag |
| | | (CA125) | 17 | |
| NAV1.2 | T_{RE}G | LICAM | XAGE1 | p53 |
| NAV1.3 | CDCP1 | LeY | Tie 2 | Mucins |
| NAV1.4 | NT5E | MSLN | MAD-CT-1 | |
| NAV1.5 | EpCAM | IL 13Ral | MAD-CT-2 | |
| CEA | gpA33 | Fos-related | L1-CAM | |
| | | antigen 1 | | |

In some embodiments, the antigen binding domain targets an antigen characteristic of a T-cell. In some embodiments, the antigen characteristic of a T-cell is selected from an exemplary T cell antigen listed in Table C2, or an antigenic fragment thereof.

TABLE C2
Exemplary T-cell Antigens

| | | | | | |
|---|---|---|---|---|---|
| a cell surface receptor | CALM1 | HLA-DRB1 | MAP2K6 (MKK6) | NFKBIA | RAF1 |
| a membrane transport | CD3D | HLA-DRB3 | MAP2K7 (MKK7) | NRAS | RELA |
| protein | (CD3δ) | | | | |
| an active or passive | CD3E | HLA-DRB4 | MAP3K1 | PAK1 | SDF1 |
| transport protein | (CD3ε) | | (MEKK1) | | |
| an ion channel protein | CD3G | HLA-DRB5 | MAP3K3 | PAK2 | SHP2 |
| | (CD3γ) | | | | |
| a pore-forming protein | CD4 | HRAS | MAP3K4 | PAK3 | SLP76 |
| a transmembrane | CD8 | IKBKA | MAP3K5 | PAK4 | SOS |
| receptor | | (CHUK) | | | |
| a membrane enzyme | CD28 | IKBKB | MAP3K8 | PIK3C2B | SRC |
| a cell adhesion protein | CD45 | IKBKE | MAP3K14 (NIK) | PIK3C3 | TBK1 |
| characteristic of a T- | | | | (VPS34) | |
| cell | | | | | |
| a G protein-coupled | CD80 | IKBKG | MAPK8 (JNK1) | PIK3CA | TCRA |
| receptor | (B7-1) | (NEMO) | | | |
| receptor tyrosine | CD86 | IL2 | MAPK9 (JNK2) | PIK3CB | TEC |
| kinase | (B7-2) | | | | |
| tyrosine kinase | CD247 | ITPR 1 | MAPK10 (JNK3) | PIK3CD | TRAF6 |
| associated receptor | (CD3ζ) | | | | |
| receptor-like tyrosine | CTLA4 | ITK | MAPK11 (p38β) | PIK3R1 | VAV1 |
| phosphatase | (CD152) | | | | |
| receptor | ELK1 | JUN | MAPK12 (p38γ) | PKCA | VAV2 |
| serine/threonine kinase | | | | | |
| receptor guanylyl | ERK1 | KRAS2 | MAPK13 (p38δ) | PKCB | ZAP70 |
| cyclase | (MAPK3) | | | | |
| histidine kinase | ERK2 | LAT | MAPK14 (p38α) | PKCM | |
| associated receptor | | | | | |
| AKTI | FOS | LCK | NCK | PKCQ | |
| AKT2 | FYN | MAP2K1 | NFAT1 | PLCY1 | |
| | | (MEK1) | | | |
| AKT3 | GRAP2 | MAP2K2 | NFAT2 | PRF1 | |
| | (GADS) | (MEK2) | | (Perforin) | |
| ATF2 | GRB2 | MAP2K3 | NFKB1 | PTEN | |
| | | (MKK3) | | | |
| BCL10 | HLA- | MAP2K4 | NFKB2 | RAC1 | |
| | DRA | (MKK4) | | | |

In some embodiments, the antigen binding domain targets an antigen characteristic of an autoimmune or inflammatory disorder. In some embodiments, the autoimmune or inflammatory disorder is selected from chronic graft-vs-host disease (GVHD), lupus, arthritis, immune complex glomerulonephritis, goodpasture, uveitis, hepatitis, systemic sclerosis or scleroderma, type I diabetes, multiple sclerosis, cold agglutinin disease, Pemphigus vulgaris, Grave's disease, autoimmune hemolytic anemia, Hemophilia A, Primary Sjogren's Syndrome, thrombotic thrombocytopenia purpura, neuromyelitis optica, Evan's syndrome, IgM mediated neuropathy, cryoglobulinemia, dermatomyositis, idiopathic thrombocytopenia, ankylosing spondylitis, bullous pemphigoid, acquired angioedema, chronic urticarial, antiphospholipid demyelinating polyneuropathy, and autoimmune thrombocytopenia or neutropenia or pure red cell aplasias, while exemplary non-limiting examples of alloimmune diseases include allosensitization (see, for example, Blazar et al., 2015, Am. J. Transplant, 15 (4): 931-41) or xenosensitization from hematopoietic or solid organ transplantation, blood transfusions, pregnancy with fetal allosensitization, neonatal alloimmune thrombocytopenia, hemolytic disease of the newborn, sensitization to foreign antigens such as can occur with replacement of inherited or acquired deficiency disorders treated with enzyme or protein replacement therapy, blood products, and gene therapy. In some embodiments, the antigen characteristic of an autoimmune or inflammatory disorder is selected from an exemplary antigen listed in Table C3, or an antigenic fragment thereof.

TABLE C3
Exemplary Autoimmune or Inflammatory Disorder Antigens

| | | | |
|---|---|---|---|
| a cell surface receptor | a G protein-coupled | receptor-like tyrosine | histidine kinase |
| | receptor | phosphatase | associated receptor |
| an ion channel-linked | receptor tyrosine | receptor serine/ | |
| receptor | kinase | threonine kinase | |
| an enzyme-linked | tyrosine kinase | receptor guanylyl | |
| receptor | associated receptor | cyclase | |

In some embodiments, a CAR antigen binding domain binds to a ligand expressed on B cells, plasma cells, plasmablasts. In some embodiments, the antigen expressed on B cells, plasma cells, or plasmablasts is selected from an exemplary antigen listed in Table C4, or an antigenic fragment thereof.

TABLE C4
Exemplary B cell, Plasma Cell, and Plasmablast Antigens

| | | | | |
|---|---|---|---|---|
| CD10 | CD24 | CD138 | TNF | LFA-1 |
| CD19 | CD27 | CD319 | interferon | CD3 gamma |
| | | | receptors | |
| CD20 | CD38 | BCMA | GM-CSF | CD5 |
| CD22 | CD45R | CD28 | ZAP-70 | CD2 |

In some embodiments, the antigen binding domain targets an antigen characteristic of an infectious disease. In some embodiments, wherein the infectious disease is selected from HIV, hepatitis B virus, hepatitis C virus, Human herpes virus, Human herpes virus 8 (HHV-8, Kaposi sarcoma-associated herpes virus (KSHV)), Human T-lymphotrophic virus-1 (HTLV-1), Merkel cell polyomavirus (MCV), Simian virus 40 (SV40), Epstein-Barr virus, CMV, human papillomavirus. In some embodiments, the antigen characteristic of an infectious disease is selected from an exemplary antigen listed in Table C5, or an antigenic fragment thereof.

TABLE C5
Exemplary Infectious Disease Antigens

| | |
|---|---|
| a cell surface receptor | receptor serine/threonine kinase |
| an ion channel-linked receptor | receptor guanylyl cyclase |
| an enzyme-linked receptor | histidine kinase associated receptor |
| a G protein-coupled receptor | HIV Env |
| receptor tyrosine kinase | gpl20 |
| tyrosine kinase associated receptor | CD4-induced epitope on HIV-1 Env |
| receptor-like tyrosine phosphatase | |

### Transmembrane Domain

In some embodiments, the CAR transmembrane domain comprises at least a transmembrane region of an exemplary transmembrane domain listed in Table C6, or a functional fragment thereof.

TABLE C6
Exemplary Transmembrane Domains

| | | | | |
|---|---|---|---|---|
| alpha chain of the T cell | CD22 | 4- | TCRα | CD80 |
| receptor | | 1BB/CD137 | | |
| beta chain of the T cell | CD33 | CD28 | TCRβ | CD86 |
| receptor | | | | |
| zeta chain of the T-cell | CD37 | CD34 | TCRζ | CD40 |
| receptor | | | | |
| CD28 | CD64 | CD4 | CD32 | CD40L/ |
| | | | | CD154 |
| CD3 epsilon | CD80 | FcERIγ | CD64 | VEGFR2 |
| CD45 | CD86 | CD16 | CD64 | FAS |
| CD4 | CD134 | OX40/CD134 | CD45 | FGFR2B |
| CD5 | CD137 | CD3ζ | CD5 | |
| CD8 | CD154 | CD3ε | CD9 | |
| CD9 | CD8α | CD3γ | CD22 | |
| CD16 | CD8β | CD3δ | CD37 | |

In some embodiments, the transmembrane domain is a CD8 transmembrane domain. In some embodiments, the CD8 transmembrane domain has an amino acid sequence of IYIWAPLAGTCGVLLLSLVITLYC (SEQ ID NO: 1102).

### Intracellular Signaling Domains

In some embodiments, the CAR comprises at least one signaling domain selected from one or more intracellular signaling domains listed in Table C7, or a functional fragment thereof. In some embodiments, the CAR comprises a first intracellular signaling domain and a second intracellular signaling domain. In some embodiments, the first intracellular signaling domain mediates downstream signaling during T-cell activation. In some embodiments, the second intracellular signaling domain is a costimulatory domain.

TABLE C7
Exemplary Intracellular Signaling Domains

| | | | |
|---|---|---|---|
| B7-1/CD80 | 4-1BB Ligand/TNFSF9 | RELT/TNFRSF19L | CD7 |
| B7-2/CD86 | BAFF/BLyS/TNFSF13B | TACI/TNFRSF13B | CD53 |
| B7-H1/PD-L1 | BAFF R/TNFRSF13C | TL1A/TNFSF15 | CD82/Kai-1 |
| B7-H2 | CD27/TNFRSF7 | TNF-alpha | CD90/Thy 1 |
| B7-H3 | CD27 Ligand/TNFSF7 | TNF RII/TNFRSF1B | CD96 |
| B7-H4 | CD30/TNFRSF8 | 2B4/CD244/SLAMF4 | CD160 |
| B7-H6 | CD30 Ligand/TNFSF8 | BLAME/SLAMF8 | CD200 |
| B7-H7 | CD40/TNFRSF5 | CD2 | CD300a/LMIR1 |
| BTLA/CD272 | CD40/TNFSF5 | CD2F-10/SLAMF9 | HLA Class I |
| CD28 | CD40 Ligand/TNFSF5 | CD48/SLAMF2 | HLA-DR |
| CTLA-4 | DR3/TNFRSF25 | CD58/LFA-3 | Ikaros |
| Gi24/VISTA/B7-H5 | GITR/TNFRSF18 | CD84/SLAMF5 | Integrin alpha |
| | | | 4/CD49d |
| ICOS/CD278 | GITR Ligand/TNFSF18 | CD229/SLAMF3 | Integrin alpha 4 beta |
| | | | 1 |
| PD-1 | HVEM/TNFRSF14 | CRACC/SLAMF7 | Integrin alpha 4 beta |
| | | | 7/LPAM-1 |
| PD-L2/B7-DC | LIGHT/TNFSF14 | NTB-A/SLAMF6 | LAG-3 |
| PDCD6 | Lymphotoxin- | SLAM/CD150 | TCL1A |
| | alpha/TNF-beta | | |
| 4- | OX40/TNFRSF4 | CD2 | TCL1B |
| 1BB/TNFSF9/CD137 | | | |
| CRTAM | CD30 | TIM-1/KIM- | CD2 |
| | | 1/HAVCR | |
| DAP12 | CD40 | TIM-4 | CD7 |
| Dectin-1/CLEC7A | PD-1 | TSLP | LIGHT |
| DPPIV/CD26 | ICOS | TSLP R | NKG2C |
| EphB6 | lymphocyte function- | lymphocyte function | B7-H3 |
| | associated antigen-1 | associated antigen-1 | |
| | (LFA-1) | (LFA-1) | |
| NKG2C | a ligand that specifically | a CD3 zeta domain | an immunoreceptor |
| | binds with CD83 | | tyrosine-based |
| | | | activation motif |
| | | | (ITAM) |
| CD27 | CD28 | 4-1BB | CD134/OX40 |

In some embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; (ii) a CD28 domain, or a 4-1BB domain, or functional variant thereof, and/or (iii) a 4-1BB domain, or a CD134 domain, or functional variant thereof. In some embodiments, the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the CAR comprises a CD28z co-stimulatory domain. In some embodiments, the CD28z co-stimulatory domain comprises an amino acid sequence of

(SEQ ID NO: 1103)
RSKRSRLLHSDYMNMTPRRPGPTRKHYQPYAPPRDFAAYRS

In some embodiments, the CAR comprises a CD3z signaling domain. In some embodiments, the CD3z signaling domain comprises an amino acid sequence of

(SEQ ID NO: 1104)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR.

In some embodiments, the CAR further comprises one or more spacers, e.g., wherein the spacer is a first spacer between the antigen binding domain and the transmembrane domain. In some embodiments, the first spacer includes at least a portion of an immunoglobulin constant region or variant or modified version thereof. In some embodiments, the spacer is a second spacer between the transmembrane domain and a signaling domain. In some embodiments, the second spacer is an oligopeptide, e.g., wherein the oligopeptide comprises glycine-serine doublets. In some embodiments, the CAR further comprises a hinge domain. In some embodiments, the hinge domain is a CD8 hinge domain. In some embodiments, the CD8 hinge domain has an amino acid sequence of

(SEQ ID NO: 1101)
TTTPAPRPPTPAPTIASQPLSLRPEACRPAAGGAVHTRGLDFACD.

### CAR Compositions, Methods of Manufacture, and Uses

Additionally provided herein is a system for modifying DNA of a mammalian cell (e.g., a T cell, e.g., a primary T cell) to express a CAR, the system comprising:
(a) a retrotransposon gene modifying polypeptide or a nucleic acid (e.g., DNA or mRNA) encoding the gene modifying polypeptide, as disclosed herein, and
(b) a template RNA (or DNA encoding the template RNA) comprising (i) a sequence that binds the polypeptide and (ii) a heterologous object sequence encoding a chimeric antigen receptor (CAR), wherein the CAR comprises an antigen-binding domain, a transmembrane domain, a first intracellular signaling domain, and a second intracellular signaling domain, as disclosed herein.

In some embodiments, the system comprises:
(a) a retrotransposon gene modifying polypeptide or a nucleic acid (e.g., DNA or mRNA) encoding the retrotransposon gene modifying polypeptide, and
(b) a template RNA (or DNA encoding the template RNA) comprising (i) a sequence that binds the polypeptide and (ii) a heterologous object sequence encoding a chimeric antigen receptor (CAR), wherein one or more of:
(i) the CAR comprises an antigen binding domain that binds one or more antigens of a blood cancer (e.g., a leukemia or lymphoma), wherein optionally the antigen is a B cell antigen;
(ii) the CAR comprises an antigen binding domain that binds one or more antigens of a solid tumor;
(iii) the CAR comprise an antigen binding domain of any one of Tables C1-C5;
(iv) the CAR comprise a linker domain of Table L1;
(v) the CAR comprises a transmembrane domain of Table C6;
(vi) the CAR comprises a hinge domain;
(vii) the CAR comprises an intracellular signaling domain of Table C7;
(viii) the CAR comprises a costimulatory domain of Table C7;
(ix) the CAR comprises an antigen binding domain which comprises an scFv; or
(x) the CAR comprises an amino acid sequence according to SEQ ID NO: 1100;
(xi) wherein the CAR comprises a first intracellular signaling domain and a second intracellular signaling domain.

In some embodiments, provided herein are a population of cells comprising immune effector cells (e.g., T cells, e.g., primary T cells) comprising a plurality of copies of a gene encoding a CAR (“a CAR gene”).

In some embodiments, at least 50%, 55%, 60%, 65%, 70%, 75%, 80%, 85%, 90%, 95%, 96%, 97%, 98%, 99%, or 100% of cells in the plurality comprises a single copy of the CAR gene. In some embodiments, each cell in the plurality comprises a single copy of the CAR gene. In some embodiments, at least 0.1% of cells in the population comprise the CAR gene.

In some embodiments, the cell population comprises one or more of cancer cells, regulatory T cells, monocytes, and NK cells. In some embodiments, the immune effector cells comprise T cells, e.g., primary T cells. In some embodiments, the immune effector cells comprise a leukapheresis sample or an apheresis sample.

Additionally provided herein are methods of modifying a mammalian cell to express a CAR. In some embodiments, the method involves contacting the cell (e.g., an immune effector cell) with a system disclosed herein. In some embodiments, the immune effector cell is a cell that expresses one or more Fc receptors and mediates one or more effector functions. In some embodiments, the immune effector cell may include, but may not be limited to, one or more of a monocyte, macrophage, neutrophil, dendritic cell, eosinophil, mast cell, platelet, large granular lymphocyte, Langerhans' cell, natural killer (NK) cell, T-lymphocyte (e.g., T-cell), a Gamma delta T cell, B-lymphocyte (e.g., B-cell) and may be from any organism including but not limited to humans, mice, rats, rabbits, and monkeys.

In some embodiments, the mammalian cell is a T cell, e.g., a primary T cell. In some embodiments, provided herein is a method of modifying the genome of a mammalian T cell (e.g., a primary T cell), the method comprising contacting the cell with:
(a) a retrotransposon gene modifying polypeptide comprising an amino acid sequence of Table R1 or a sequence at least 70%, 75%, 80%, 85%, 90%, 95%, 96%, 97%, 98%, or 99% identical thereto, or a nucleic acid (e.g., DNA or mRNA) encoding the retrotransposon gene modifying polypeptide, and
(b) a template RNA (or DNA encoding the template RNA) comprising (i) a sequence that binds the polypeptide and (ii) a heterologous object sequence (e.g., encoding a CAR).

In some embodiments, provided herein is a method of modifying the genome of a mammalian T cell (e.g., a primary T cell), the method comprising contacting the cell with:
(a) a retrotransposon gene modifying polypeptide comprising an amino acid sequence of Table R1 or a sequence having no more than 10, 20, 30, 40, 50, 60, 70, 80, 90, or 100 amino acid differences thereto, or a nucleic acid (e.g., DNA or mRNA) encoding the retrotransposon gene modifying polypeptide, and
(b) a template RNA (or DNA encoding the template RNA) comprising (i) a sequence that binds the polypeptide and (ii) a heterologous object sequence (e.g., encoding a CAR).

In some embodiments, the method is performed ex vivo or in vitro.

Additionally provided herein is a reaction mixture comprising a retrotransposon gene modifying system disclosed herein (e.g., comprising a heterologous object sequence encoding a chimeric antigen receptor (CAR)) and a mammalian cell (e.g., a T cell, e.g., a primary T cell).

In some embodiments, the reaction mixture comprises a retrotransposon gene modifying polypeptide comprising an amino acid sequence of Table R1 or a sequence at least 70%, 75%, 80%, 85%, 90%, 95%, 96%, 97%, 98%, or 99% identical thereto, or a nucleic acid (e.g., DNA or mRNA) encoding the retrotransposon gene modifying polypeptide and a mammalian T cell (e.g., a primary T cell). In some embodiments, the reaction mixture additionally includes a template RNA (or DNA encoding the template RNA) comprising (i) a sequence that binds the polypeptide and (ii) a heterologous object sequence.

In some embodiments, the reaction mixture comprises a retrotransposon gene modifying polypeptide comprising an amino acid sequence of Table R1 or a sequence no more than 10, 20, 30, 40, 50, 60, 70, 80, 90, or 100 amino acid differences thereto, or a nucleic acid (e.g., DNA or mRNA) encoding the retrotransposon gene modifying polypeptide and a mammalian T cell (e.g., a primary T cell). In some embodiments, the reaction mixture additionally includes a template RNA (or DNA encoding the template RNA) comprising (i) a sequence that binds the polypeptide and (ii) a heterologous object sequence.

In some embodiments, the systems, reaction mixtures, and cell populations modified using the methods disclosed herein can be used to treat a subject in need thereof. In some embodiments, the subject has a cancer, e.g., a hematological cancer or a solid tumor. In some embodiments, the subject has an infectious disease. In other embodiments, the subject has an autoimmune or an inflammatory disease.

### Compositions and Methods for Modifying Mammalian Cells

In some embodiments, provided herein are methods of modifying mammalian cells and reaction mixtures and systems for the same.

In some embodiments, provided herein is a method of modifying the genome of a mammalian T cell (e.g., a primary T cell), the method comprising contacting the cell with:
(a) a retrotransposon gene modifying polypeptide comprising an amino acid sequence of Table R1 or a sequence at least 70%, 75%, 80%, 85%, 90%, 95%, 96%, 97%, 98%, or 99% identical thereto, or a nucleic acid (e.g., DNA or mRNA) encoding the retrotransposon gene modifying polypeptide, and
(b) a template RNA (or DNA encoding the template RNA) comprising (i) a sequence that binds the polypeptide and (ii) a heterologous object sequence.

In some embodiments, provided herein is a method of modifying the genome of a mammalian T cell (e.g., a primary T cell), the method comprising contacting the cell with a system comprising:
(a) a retrotransposon gene modifying polypeptide comprising an amino acid sequence of Table R1 or a sequence having no more than 10, 20, 30, 40, 50, 60, 70, 80, 90, or 100 amino acid differences thereto, or a nucleic acid (e.g., DNA or mRNA) encoding the retrotransposon gene modifying polypeptide, and
(b) a template RNA (or DNA encoding the template RNA) comprising (i) a sequence that binds the polypeptide and (ii) a heterologous object sequence.

In some embodiments, the method is performed ex vivo or in vitro. In some embodiments, the retrotransposon gene modifying polypeptide and/or template RNA are formulated with an LNP.

In some embodiments, contacting the cell with the system results in insertion of the heterologous object sequence into at least 1%, 5%, 10%, 15%, 20%, 25%, or 30% of T cells. In some embodiments, modifying a genome of a cell (e.g., a primary cell, e.g., a T cell) using a retrotransposon gene modifying system does not result in activation of the endogenous DNA damage response (DDR) pathway. In some embodiments, modifying a genome of a cell (e.g., a primary cell) using a retrotransposon gene modifying system results in activation of the cell's endogenous DDR pathway less than in an otherwise similar cell treated with Cas9.

In some embodiments, modifying a genome of a cell (e.g., a primary cell, e.g., a T cell) using a retrotransposon gene modifying system does not result in activation of the endogenous interferon response. In some embodiments, modifying a genome of a cell (e.g., a primary cell, e.g., a T cell) using a retrotransposon gene modifying system results in activation of the cell's interferon response less than in an otherwise similar cell treated with a retrotransposon gene modifying system comprising elements from a LINE-1 retrotransposase.

### Administration and Delivery

The compositions and systems described herein may be used in vitro or in vivo. In some embodiments the system or components of the system are delivered to cells (e.g., mammalian cells, e.g., human cells), e.g., in vitro or in vivo. In some embodiments, the cells are eukaryotic cells, e.g., cells of a multicellular organism, e.g., an animal, e.g., a mammal (e.g., human, swine, bovine), a bird (e.g., poultry, such as chicken, turkey, or duck), or a fish. In some embodiments, the cells are non-human animal cells (e.g., a laboratory animal, a livestock animal, or a companion animal). In some embodiments, the cell is a stem cell (e.g., a hematopoietic stem cell), a fibroblast, or a T cell. In some embodiments, the cell is an immune cell, e.g., a T cell (e.g., a Treg, CD4, CD8, γδ, or memory T cell), B cell (e.g., memory B cell or plasma cell), or NK cell. In some embodiments, the cell is an immune effector cell. In some embodiments, the cell is a T cell, e.g., a primary T cell. In some embodiments, the cell is a non-dividing cell, e.g., a non-dividing fibroblast or non-dividing T cell. In some embodiments, the cell is an HSC and p53 is not upregulated or is upregulated by less than 10%, 5%, 2%, or 1%, e.g., as determined according to the method described in Example 30 of PCT/US2019/048607. The skilled artisan will understand that the components of the heterologous or retrotransposon gene modifying system may be delivered in the form of polypeptide, nucleic acid (e.g., DNA, RNA), and combinations thereof.

In one embodiment the system and/or components of the system are delivered as nucleic acid. For example, the gene modifying polypeptide may be delivered in the form of a DNA or RNA encoding the polypeptide, and the template RNA may be delivered in the form of RNA or its complementary DNA to be transcribed into RNA. In some embodiments the system or components of the system are delivered on 1, 2, 3, 4, or more distinct nucleic acid molecules. In some embodiments the system or components of the system are delivered as a combination of DNA and RNA. In some embodiments the system or components of the system are delivered as a combination of DNA and protein. In some embodiments the system or components of the system are delivered as a combination of RNA and protein. In some embodiments the gene modifying polypeptide is delivered as a protein.

In some embodiments the system or components of the system are delivered to cells, e.g. mammalian cells or human cells, using a vector. The vector may be, e.g., a plasmid or a virus. In some embodiments, delivery is in vivo, in vitro, ex vivo, or in situ. In some embodiments the virus is an adeno associated virus (AAV), a lentivirus, or an adenovirus. In some embodiments the system or components of the system are delivered to cells with a viral-like particle or a virosome. In some embodiments the delivery uses more than one virus, viral-like particle or virosome.

In one embodiment, the compositions and systems described herein can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi: 10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi: 10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

A variety of nanoparticles can be used for delivery, such as a liposome, a lipid nanoparticle, a cationic lipid nanoparticle, an ionizable lipid nanoparticle, a polymeric nanoparticle, a gold nanoparticle, a dendrimer, a cyclodextrin nanoparticle, a micelle, or a combination of the foregoing.

Lipid nanoparticles are an example of a carrier that provides a biocompatible and biodegradable delivery system for the pharmaceutical compositions described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi: 10.3390/nano7060122.

Exosomes can also be used as drug delivery vehicles for the compositions and systems described herein. For a review, see Ha et al. July 2016. Acta Pharmaceutica Sinica B. Volume 6, Issue 4, Pages 287-296; doi.org/10.1016/j.apsb.2016.02.001.

Fusosomes interact and fuse with target cells, and thus can be used as delivery vehicles for a variety of molecules. They generally consist of a bilayer of amphipathic lipids enclosing a lumen or cavity and a fusogen that interacts with the amphipathic lipid bilayer. The fusogen component has been shown to be engineerable in order to confer target cell specificity for the fusion and payload delivery, allowing the creation of delivery vehicles with programmable cell specificity (see for example Patent Application WO2020014209, the teachings of which relating to fusosome design, preparation, and usage are incorporated herein by reference).

In some embodiments, the protein component(s) of the gene modifying system may be pre-associated with the template nucleic acid (e.g., template RNA). For example, in some embodiments, the gene modifying polypeptide may be first combined with the template nucleic acid (e.g., template RNA) to form a ribonucleoprotein (RNP) complex. In some embodiments, the RNP may be delivered to cells via, e.g., transfection, nucleofection, virus, vesicle, LNP, exosome, fusosome.

A gene modifying system can be introduced into cells, tissues and multicellular organisms. In some embodiments the system or components of the system are delivered to the cells via mechanical means or physical means.

Formulation of protein therapeutics is described in Meyer (Ed.), Therapeutic Protein Drug Products: Practical Approaches to formulation in the Laboratory, Manufacturing, and the Clinic, Woodhead Publishing Series (2012).

### Tissue Specific Activity/Administration

In some embodiments, a system described herein can make use of one or more feature (e.g., a promoter or microRNA binding site) to limit activity in off-target cells or tissues.

In some embodiments, a nucleic acid described herein (e.g., a template RNA or a DNA encoding a template RNA) comprises a promoter sequence, e.g., a tissue specific promoter sequence. In some embodiments, the tissue-specific promoter is used to increase the target-cell specificity of a gene modifying system. For instance, the promoter can be chosen on the basis that it is active in a target cell type but not active in (or active at a lower level in) a non-target cell type. Thus, even if the promoter integrated into the genome of a non-target cell, it would not drive expression (or only drive low level expression) of an integrated gene. A system having a tissue-specific promoter sequence in the template RNA may also be used in combination with a microRNA binding site, e.g., in the template RNA or a nucleic acid encoding a gene modifying protein, e.g., as described herein. A system having a tissue-specific promoter sequence in the template RNA may also be used in combination with a DNA encoding a gene modifying polypeptide, driven by a tissue-specific promoter, e.g., to achieve higher levels of gene modifying protein in target cells than in non-target cells. In some embodiments, e.g., for liver indications, a tissue-specific promoter is selected from Table 3 of WO2020014209, incorporated herein by reference.

In some embodiments, a nucleic acid described herein (e.g., a template RNA or a DNA encoding a template RNA) comprises a microRNA binding site. In some embodiments, the microRNA binding site is used to increase the target-cell specificity of a gene modifying system. For instance, the microRNA binding site can be chosen on the basis that is recognized by a miRNA that is present in a non-target cell type, but that is not present (or is present at a reduced level relative to the non-target cell) in a target cell type. Thus, when the template RNA is present in a non-target cell, it would be bound by the miRNA, and when the template RNA is present in a target cell, it would not be bound by the miRNA (or bound but at reduced levels relative to the non-target cell). While not wishing to be bound by theory, binding of the miRNA to the template RNA may interfere with its activity, e.g., may interfere with insertion of the heterologous object sequence into the genome. Accordingly, the system would edit the genome of target cells more efficiently than it edits the genome of non-target cells, e.g., the heterologous object sequence would be inserted into the genome of target cells more efficiently than into the genome of non-target cells, or an insertion or deletion is produced more efficiently in target cells than in non-target cells. A system having a microRNA binding site in the template RNA (or DNA encoding it) may also be used in combination with a nucleic acid encoding a gene modifying polypeptide, wherein expression of the gene modifying polypeptide is regulated by a second microRNA binding site, e.g., as described herein. In some embodiments, e.g., for liver indications, a miRNA is selected from Table 4 of WO2020014209, incorporated herein by reference.

In some embodiments, the template RNA comprises a microRNA sequence, an siRNA sequence, a guide RNA sequence, or a piwi RNA sequence.

### Promoters

In some embodiments, one or more promoter or enhancer elements are operably linked to a nucleic acid encoding a gene modifying protein or a template nucleic acid, e.g., that controls expression of the heterologous object sequence. In certain embodiments, the one or more promoter or enhancer elements comprise cell-type or tissue specific elements. In some embodiments, the promoter or enhancer is the same or derived from the promoter or enhancer that naturally controls expression of the heterologous object sequence. For example, the ornithine transcarbomylase promoter and enhancer may be used to control expression of the ornithine transcarbomylase gene in a system or method provided by the invention for correcting ornithine transcarbomylase deficiencies. In some embodiments, the promoter is a promoter of Table 16 or 17 or a functional fragment or variant thereof.

Exemplary tissue specific promoters that are commercially available can be found, for example, at a uniform resource locator (e.g., invivogen.com/tissue-specific-promoters). In some embodiments, a promoter is a native promoter or a minimal promoter, e.g., which consists of a single fragment from the 5′ region of a given gene. In some embodiments, a native promoter comprises a core promoter and its natural 5′ UTR. In some embodiments, the 5′ UTR comprises an intron. In other embodiments, these include composite promoters, which combine promoter elements of different origins or were generated by assembling a distal enhancer with a minimal promoter of the same origin.

Exemplary cell or tissue specific promoters are provided in the tables, below, and exemplary nucleic acid sequences encoding them are known in the art and can be readily accessed using a variety of resources, such as the NCBI database, including RefSeq, as well as the Eukaryotic Promoter Database (/epd.epfl.ch//index.php)

TABLE 16
Exemplary cell or tissue-specific promoters

| Promoter | Target cells |
|---|---|
| B29 Promoter | B cells |
| CD14 Promoter | Monocytic Cells |
| CD43 Promoter | Leukocytes and platelets |
| CD45 Promoter | Hematopoeitic cells |
| CD68 promoter | macrophages |
| Desmin promoter | muscle cells |
| Elastase-1 | pancreatic acinar cells |
| promoter | |
| Endoglin promoter | endothelial cells |
| fibronectin | differentiating cells, healing |
| promoter | tissue |
| Flt-1 promoter | endothelial cells |
| GFAP promoter | Astrocytes |
| GPIIB promoter | megakaryocytes |
| ICAM-2 Promoter | Endothelial cells |
| INF-Beta promoter | Hematopoeitic cells |
| Mb promoter | muscle cells |
| Nphs1 promoter | podocytes |
| OG-2 promoter | Osteoblasts, Odonblasts |
| SP-B promoter | Lung |
| Syn1 promoter | Neurons |
| WASP promoter | Hematopoeitic cells |
| SV40/bAlb | Liver |
| promoter | |
| SV40/bAlb | Liver |
| promoter | |
| SV40/Cd3 | Leukocytes and platelets |
| promoter | |
| SV40/CD45 | hematopoeitic cells |
| promoter | |
| NSE/RU5' | Mature Neurons |
| promoter | |

TABLE 17

| | | |
|---|---|---|
| Promoter | Gene Description | Gene Specificity |

Additional exemplary cell or tissue-specific promoters

| | | |
|---|---|---|
| APOA2 | Apolipoprotein A-II | Hepatocytes (from hepatocyte |
| | | progenitors) |
| SERPINA | Serpin peptidase inhibitor, clade A | Hepatocytes |
| 1 (hAAT) | (alpha-1 | (from definitive endoderm |
| | antiproteinase, antitrypsin), member 1 | stage) |
| | (also named alpha 1 anti-tryps in) | |
| CYP3A | Cytochrome P450, family 3, | Mature Hepatocytes |
| | subfamily A, polypeptide | |
| MIR122 | MicroRNA 122 | Hepatocytes |
| | | (from early stage embryonic |
| | | liver cells) |
| | | and endoderm |

Pancreatic specific promoters

| | | |
|---|---|---|
| INS | Insulin | Pancreatic beta cells |
| | | (from definitive endoderm stage) |
| IRS2 | Insulin receptor substrate 2 | Pancreatic beta cells |
| Pdx1 | Pancreatic and duodenal | Pancreas |
| | homeobox 1 | (from definitive endoderm stage) |
| Alx3 | Aristaless-like homeobox 3 | Pancreatic beta cells |
| | | (from definitive endoderm stage) |
| Ppy | Pancreatic polypeptide | PP pancreatic cells |
| | | (gamma cells) |

Cardiac specific promoters

| | | |
|---|---|---|
| Myh6 | Myosin, heavy chain 6, cardiac | Late differentiation marker of cardiac |
| (aMHC) | muscle, alpha | muscle cells (atrial specificity) |
| MYL2 | Myosin, light chain 2, regulatory, | Late differentiation marker of cardiac |
| (MLC-2v) | cardiac, slow | muscle cells (ventricular specificity) |
| ITNN13 | Troponin I type 3 (cardiac) | Cardiomyocytes |
| (cTnl) | | (from immature state) |
| ITNN13 | Troponin I type 3 (cardiac) | Cardiomyocytes |
| (cTnl) | | (from immature state) |
| NPPA | Natriuretic peptide precursor A (also | Atrial specificity in adult cells |
| (ANF) | named Atrial Natriuretic Factor) | |
| Slc8a1 | Solute carrier family 8 | Cardiomyocytes from early |
| (Ncx1) | (sodium/calcium exchanger), member | developmental stages |
| | 1 | |

CNS specific promoters

| | | |
|---|---|---|
| SYN1 | Synapsin I | Neurons |
| (hSyn) | | |
| GFAP | Glial fibrillary acidic protein | Astrocytes |
| INA | Internexin neuronal intermediate | Neuroprogenitors |
| | filament protein, alpha (a-internexin) | |
| NES | Nestin | Neuroprogenitors and ectoderm |
| MOBP | Myelin-associated oligodendrocyte | Oligodendrocytes |
| | basic protein | |
| MBP | Myelin basic protein | Oligodendrocytes |
| TH | Tyrosine hydroxylase | Dopaminergic neurons |
| FOXA2 | Forkhead box A2 | Dopaminergic neurons (also used as a |
| (HNF3 | | marker of endoderm) |
| beta) | | |

Skin specific promoters

| | | |
|---|---|---|
| FLG | Filaggrin | Keratinocytes from granular layer |
| K14 | Keratin 14 | Keratinocytes from granular |
| | | and basal layers |
| TGM3 | Transglutaminase 3 | Keratinocytes from granular layer |

Immune cell specific promoters

| | | |
|---|---|---|
| ITGAM | Integrin, alpha M (complement | Monocytes, macrophages , granulocytes, |
| (CD11B) | component 3 receptor 3 subunit) | natural killer cells |

Urogential cell specific promoters

| | | |
|---|---|---|
| Pbsn | Probasin | Prostatic epithelium |
| Upk2 | Uroplakin 2 | Bladder |
| Sbp | Spermine binding protein | Prostate |
| Fer114 | Fer-1-like 4 | Bladder |

Endothelial cell specific promoters

| | | |
|---|---|---|
| ENG | Endoglin | Endothelial cells |

Pluripotent and embryonic cell specific promoters

| | | |
|---|---|---|
| Oct4 | POU class 5 homeobox 1 | Pluripotent cells |
| (POU5F1) | | (germ cells, ES cells, iPS cells) |
| NANOG | Nanog homeobox | Pluripotent cells |
| | | (ES cells, iPS cells) |
| Synthetic | Synthetic promoter based on a Oct-4 | Pluripotent cells (ES cells, iPS cells) |
| Oct4 | core enhancer element | |
| T | Brachyury | Mesoderm |
| brachyury | | |
| NES | Nestin | Neuroprogenitors and Ectoderm |
| SOX17 | SRY (sex determining region Y)-box | Endoderm |
| | 17 | |
| FOXA2 | Forkhead box A2 | Endoderm (also used as a marker of |
| (HNFJ | | dopaminergic neurons) |
| beta) | | |
| MIR122 | MicroRNA 122 | Endoderm and hepatocytes |
| | | (from early stage embryonic liver cells~ |

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544; incorporated herein by reference in its entirety).

In some embodiments, a nucleic acid encoding a gene modifying protein or template nucleic acid is operably linked to a control element, e.g., a transcriptional control element, such as a promoter. The transcriptional control element may, in some embodiment, be functional in either a eukaryotic cell, e.g., a mammalian cell; or a prokaryotic cell (e.g., bacterial or archaeal cell). In some embodiments, a nucleotide sequence encoding a polypeptide is operably linked to multiple control elements, e.g., that allow expression of the nucleotide sequence encoding the polypeptide in both prokaryotic and eukaryotic cells.

For illustration purposes, examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific enolase (NSE) promoter (see, e.g., EMBL HSENO2, X51956); an aromatic amino acid decarboxylase (AADC) promoter, a neurofilament promoter (see, e.g., GenBank HUMNFL, L04147); a synapsin promoter (see, e.g., GenBank HUMSYNIB, M55301); a thy-1 promoter (see, e.g., Chen et al. (1987) Cell 51:7-19; and Llewellyn, et al. (2010) Nat. Med. 16 (10): 1161-1166), a serotonin receptor promoter (see, e.g., GenBank S62283); a tyrosine hydroxylase promoter (TH) (see, e.g., Oh et al. (2009) Gene Ther 16:437; Sasaoka et al. (1992) Mol. Brain Res. 16:274; Boundy et al. (1998) J. Neurosci. 18:9989; and Kaneda et al. (1991) Neuron 6:583-594); a GnRH promoter (see, e.g., Radovick et al. (1991) Proc. Natl. Acad Sci. USA 88:3402-3406), an L7 promoter (see, e.g., Oberdick et al. (1990) Science 248:223-226); a DNMT promoter (see, e.g., Bartge et al. (1988) Proc. Natl. Acad. Sci. USA 85:3648-3652); an enkephalin promoter (see, e.g., Comb et al. (1988) EMBO J. 17:3793-3805); a myelin basic protein (MBP) promoter; a Ca2+-calmodulin-dependent protein kinase II-alpha (CamKIIα) promoter (see, e.g., Mayford et al. (1996) Proc. Natl. Acad. Sci. USA 93:13250; and Casanova et al. (2001) Genesis 31:37), a CMV enhancer/platelet-derived growth factor-β promoter (see, e.g., Liu et al. (2004) Gene Therapy 11:52-60); and the like.

Adipocyte-specific spatially restricted promoters include, but are not limited to, the aP2 gene promoter/enhancer, e.g., a region from −5.4 kb to +21 bp of a human aP2 gene (see, e.g., Tozzo et al. (1997) Endocrinol. 138:1604; Ross et al. (1990) Proc. Natl. Acad. Sci. USA 87:9590; and Pavjani et al. (2005) Nat. Med. 11:797); a glucose transporter-4 (GLUT4) promoter (see, e.g., Knight et al. (2003) Proc. Natl. Acad. Sci. USA 100:14725); a fatty acid translocase (FAT/CD36) promoter (see, e.g., Kuriki et al. (2002) Biol. Pharm. Bull. 25:1476; and Sato et al. (2002) J. Biol. Chem. 277:15703); a stearoyl-CoA desaturase-1 (SCD1) promoter (Tabor et al. (1999) J. Biol. Chem. 274:20603); a leptin promoter (see, e.g., Mason et al. (1998) Endocrinol. 139:1013; and Chen et al. (1999) Biochem. Biophys. Res. Comm. 262:187); an adiponectin promoter (see, e.g., Kita et al. (2005) Biochem Biophys. Res. Comm. 331:484; and Chakrabarti (2010) Endocrinol. 151:2408); an adipsin promoter (see, e.g., Platt et al. (1989) Proc. Natl. Acad. Sci. USA 86:7490); a resistin promoter (see, e.g., Seo et al. (2003) Molec. Endocrinol. 17:1522); and the like.

Cardiomyocyte-specific spatially restricted promoters include, but are not limited to, control sequences derived from the following genes: myosin light chain-2, α-myosin heavy chain, AE3, cardiac troponin C, cardiac actin, and the like. Franz et al. (1997) Cardiovasc. Res. 35:560-566; Robbins et al. (1995) Ann. N.Y. Acad. Sci. 752:492-505, Linn et al. (1995) Circ. Res. 76:584-591; Parmacek et al. (1994) Mol. Cell. Biol. 14:1870-1885; Hunter et al. (1993) Hypertension 22:608-617, and Sartorelli et al. (1992) Proc. Natl. Acad. Sci. USA 89:4047-4051.

Smooth muscle-specific spatially restricted promoters include, but are not limited to, an SM220 promoter (see, e.g., Akyürek et al. (2000) Mol. Med. 6:983; and U.S. Pat. No. 7,169,874); a smoothelin promoter (see, e.g., WO 2001/018048); an α-smooth muscle actin promoter; and the like. For example, a 0.4 kb region of the SM220 promoter, within which lie two CArG elements, has been shown to mediate vascular smooth muscle cell-specific expression (see, e.g., Kim, et al. (1997) Mol. Cell. Biol. 17, 2266-2278; Li, et al., (1996) J. Cell Biol. 132, 849-859; and Moessler, et al. (1996) Development 122, 2415-2425).

Photoreceptor-specific spatially restricted promoters include, but are not limited to, a rhodopsin promoter; a rhodopsin kinase promoter (Young et al. (2003) Ophthalmol. Vis. Sci. 44:4076); a beta phosphodiesterase gene promoter (Nicoud et al. (2007) J. Gene Med. 9:1015); a retinitis pigmentosa gene promoter (Nicoud et al. (2007) supra); an interphotoreceptor retinoid-binding protein (IRBP) gene enhancer (Nicoud et al. (2007) supra); an IRBP gene promoter (Yokoyama et al. (1992) Exp Eye Res. 55:225); and the like.

In some embodiments, a heterologous or retrotransposon gene modifying system, e.g., DNA encoding a gene modifying polypeptide, DNA encoding a template RNA, or DNA or RNA encoding a heterologous object sequence, is designed such that one or more elements is operably linked to a tissue-specific promoter, e.g., a promoter that is active in T-cells. In further embodiments, the T-cell active promoter is inactive in other cell types, e.g., B-cells, NK cells. In some embodiments, the T-cell active promoter is derived from a promoter for a gene encoding a component of the T-cell receptor, e.g., TRAC, TRBC, TRGC, TRDC. In some embodiments, the T-cell active promoter is derived from a promoter for a gene encoding a component of a T-cell-specific cluster of differentiation protein, e.g., CD3, e.g., CD3D, CD3E, CD3G, CD3Z. In some embodiments, T-cell-specific promoters in gene modifying systems are discovered by comparing publicly available gene expression data across cell types and selecting promoters from the genes with enhanced expression in T-cells. In some embodiments, promoters may be selecting depending on the desired expression breadth, e.g., promoters that are active in T-cells only, promoters that are active in NK cells only, promoters that are active in both T-cells and NK cells.

Cell-specific promoters known in the art may be used to direct expression of a gene modifying protein, e.g., as described herein. Nonlimiting exemplary mammalian cell-specific promoters have been characterized and used in mice expressing Cre recombinase in a cell-specific manner. Certain nonlimiting exemplary mammalian cell-specific promoters are listed in Table 1 of U.S. Pat. No. 9,845,481, incorporated herein by reference.

In some embodiments, a vector as described herein comprises an expression cassette. Typically, an expression cassette comprises the nucleic acid molecule of the instant invention operatively linked to a promoter sequence. For example, a promoter is operatively linked with a coding sequence when it is capable of affecting the expression of that coding sequence (e.g., the coding sequence is under the transcriptional control of the promoter). Encoding sequences can be operatively linked to regulatory sequences in sense or antisense orientation. In certain embodiments, the promoter is a heterologous promoter. In certain embodiments, an expression cassette may comprise additional elements, for example, an intron, an enhancer, a polyadenylation site, a woodchuck response element (WRE), and/or other elements known to affect expression levels of the encoding sequence. A promoter typically controls the expression of a coding sequence or functional RNA. In certain embodiments, a promoter sequence comprises proximal and more distal upstream elements and can further comprise an enhancer element. An enhancer can typically stimulate promoter activity and may be an innate element of the promoter or a heterologous element inserted to enhance the level or tissue-specificity of a promoter. In certain embodiments, the promoter is derived in its entirety from a native gene. In certain embodiments, the promoter is composed of different elements derived from different naturally occurring promoters. In certain embodiments, the promoter comprises a synthetic nucleotide sequence. It will be understood by those skilled in the art that different promoters will direct the expression of a gene in different tissues or cell types, or at different stages of development, or in response to different environmental conditions or to the presence or the absence of a drug or transcriptional co-factor. Ubiquitous, cell-type-specific, tissue-specific, developmental stage-specific, and conditional promoters, for example, drug-responsive promoters (e.g., tetracycline-responsive promoters) are well known to those of skill in the art. Exemplary promoters include, but are not limited to, the phosphoglycerate kinase (PKG) promoter, CAG (composite of the CMV enhancer the chicken beta actin promoter (CBA) and the rabbit beta globin intron), NSE (neuronal specific enolase), synapsin or NeuN promoters, the SV40 early promoter, mouse mammary tumor virus LTR promoter, adenovirus major late promoter (Ad MLP), a herpes simplex virus (HSV) promoter, a cytomegalovirus (CMV) promoter such as the CMV immediate early promoter region (CMVIE), SFFV promoter, rous sarcoma virus (RSV) promoter, synthetic promoters, hybrid promoters, and the like. Other promoters can be of human origin or from other species, including from mice. Common promoters include, e.g., the human cytomegalovirus (CMV) immediate early gene promoter, the SV40 early promoter, the Rous sarcoma virus long terminal repeat, [beta]-action, rat insulin promoter, the phosphoglycerate kinase promoter, the human alpha-1 antitrypsin (hAAT) promoter, the transthyretin promoter, the TBG promoter and other liver-specific promoters, the desmin promoter and similar muscle-specific promoters, the EF1-alpha promoter, hybrid promoters with multi-tissue specificity, promoters specific for neurons like synapsin and glyceraldehyde-3-phosphate dehydrogenase promoter, all of which are promoters well known and readily available to those of skill in the art, can be used to obtain high-level expression of the coding sequence of interest. In addition, sequences derived from non-viral genes, such as the murine metallothionein gene, will also find use herein. Such promoter sequences are commercially available from, e.g., Stratagene (San Diego, CA). Additional exemplary promoter sequences are described, for example, in WO2018213786A1 (incorporated by reference herein in its entirety).

In some embodiments, the apolipoprotein E enhancer (ApoE) or a functional fragment thereof is used, e.g., to drive expression in the liver. In some embodiments, two copies of the ApoE enhancer or a functional fragment thereof are used. In some embodiments, the ApoE enhancer or functional fragment thereof is used in combination with a promoter, e.g., the human alpha-1 antitrypsin (hAAT) promoter.

In some embodiments, the regulatory sequences impart tissue-specific gene expression capabilities. In some cases, the tissue-specific regulatory sequences bind tissue-specific transcription factors that induce transcription in a tissue specific manner. Various tissue-specific regulatory sequences (e.g., promoters, enhancers, etc.) are known in the art. Exemplary tissue-specific regulatory sequences include, but are not limited to, the following tissue-specific promoters: a liver-specific thyroxin binding globulin (TBG) promoter, a insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)), bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991), and the neuron-specific vgf gene promoter (Piccioli et al., Neuron, 15:373-84 (1995), and others. Additional exemplary promoter sequences are described, for example, in U.S. Pat. No. 10,300,146 (incorporated herein by reference in its entirety). In some embodiments, a tissue-specific regulatory element, e.g., a tissue-specific promoter, is selected from one known to be operably linked to a gene that is highly expressed in a given tissue, e.g., as measured by RNA-seq or protein expression data, or a combination thereof. Methods for analyzing tissue specificity by expression are taught in Fagerberg et al. Mol Cell Proteomics 13 (2): 397-406 (2014), which is incorporated herein by reference in its entirety

In some embodiments, a vector described herein is a multicistronic expression construct. Multicistronic expression constructs include, for example, constructs harboring a first expression cassette, e.g. comprising a first promoter and a first encoding nucleic acid sequence, and a second expression cassette, e.g. comprising a second promoter and a second encoding nucleic acid sequence. Such multicistronic expression constructs may, in some instances, be particularly useful in the delivery of non-translated gene products, such as hairpin RNAs, together with a polypeptide, for example, a gene modifying polypeptide and gene modifying template. In some embodiments, multicistronic expression constructs may exhibit reduced expression levels of one or more of the included transgenes, for example, because of promoter interference or the presence of incompatible nucleic acid elements in close proximity. If a multicistronic expression construct is part of a viral vector, the presence of a self-complementary nucleic acid sequence may, in some instances, interfere with the formation of structures necessary for viral reproduction or packaging.

In some embodiments, the sequence encodes an RNA with a hairpin. In some embodiments, the hairpin RNA is a guide RNA, a template RNA, a shRNA, or a microRNA. In some embodiments, the first promoter is an RNA polymerase I promoter. In some embodiments, the first promoter is an RNA polymerase II promoter. In some embodiments, the second promoter is an RNA polymerase III promoter. In some embodiments, the second promoter is a U6 or H1 promoter.

Without wishing to be bound by theory, multicistronic expression constructs may not achieve optimal expression levels as compared to expression systems containing only one cistron. One of the suggested causes of lower expression levels achieved with multicistronic expression constructs comprising two or more promoter elements is the phenomenon of promoter interference (see, e.g., Curtin J A, Dane A P, Swanson A, Alexander I E, Ginn S L. Bidirectional promoter interference between two widely used internal heterologous promoters in a late-generation lentiviral construct. Gene Ther. 2008 March; 15 (5): 384-90; and Martin-Duque P, Jezzard S, Kaftansis L, Vassaux G. Direct comparison of the insulating properties of two genetic elements in an adenoviral vector containing two different expression cassettes. Hum Gene Ther. 2004 October; 15 (10): 995-1002; both references incorporated herein by reference for disclosure of promoter interference phenomenon). In some embodiments, the problem of promoter interference may be overcome, e.g., by producing multicistronic expression constructs comprising only one promoter driving transcription of multiple encoding nucleic acid sequences separated by internal ribosomal entry sites, or by separating cistrons comprising their own promoter with transcriptional insulator elements. In some embodiments, single-promoter driven expression of multiple cistrons may result in uneven expression levels of the cistrons. In some embodiments, a promoter cannot efficiently be isolated and isolation elements may not be compatible with some gene transfer vectors, for example, some retroviral vectors.

### MicroRNAs

MicroRNAs (miRNAs) and other small interfering nucleic acids generally regulate gene expression via target RNA transcript cleavage/degradation or translational repression of the target messenger RNA (mRNA). miRNAs may, in some instances, be natively expressed, typically as final 19-25 non-translated RNA products. miRNAs generally exhibit their activity through sequence-specific interactions with the 3′ untranslated regions (UTR) of target mRNAs. These endogenously expressed miRNAs may form hairpin precursors that are subsequently processed into an miRNA duplex, and further into a mature single stranded miRNA molecule. This mature miRNA generally guides a multiprotein complex, miRISC, which identifies target 3′ UTR regions of target mRNAs based upon their complementarity to the mature miRNA. Useful transgene products may include, for example, miRNAs or miRNA binding sites that regulate the expression of a linked polypeptide. A non-limiting list of miRNA genes; the products of these genes and their homologues are useful as transgenes or as targets for small interfering nucleic acids (e.g., miRNA sponges, antisense oligonucleotides), e.g., in methods such as those listed in U.S. Pat. No. 10,300,146, 22:25-25:48, are herein incorporated by reference. In some embodiments, one or more binding sites for one or more of the foregoing miRNAs are incorporated in a transgene, e.g., a transgene delivered by a rAAV vector, e.g., to inhibit the expression of the transgene in one or more tissues of an animal harboring the transgene. In some embodiments, a binding site may be selected to control the expression of a transgene in a tissue specific manner. For example, binding sites for the liver-specific miR-122 may be incorporated into a transgene to inhibit expression of that transgene in the liver. Additional exemplary miRNA sequences are described, for example, in U.S. Pat. No. 10,300,146 (incorporated herein by reference in its entirety).

An miR inhibitor or miRNA inhibitor is generally an agent that blocks miRNA expression and/or processing. Examples of such agents include, but are not limited to, microRNA antagonists, microRNA specific antisense, microRNA sponges, and microRNA oligonucleotides (double-stranded, hairpin, short oligonucleotides) that inhibit miRNA interaction with a Drosha complex. MicroRNA inhibitors, e.g., miRNA sponges, can be expressed in cells from transgenes (e.g., as described in Ebert, M. S. Nature Methods, Epub Aug. 12, 2007; incorporated by reference herein in its entirety). In some embodiments, microRNA sponges, or other miR inhibitors, are used with the AAVs. microRNA sponges generally specifically inhibit miRNAs through a complementary heptameric seed sequence. In some embodiments, an entire family of miRNAs can be silenced using a single sponge sequence. Other methods for silencing miRNA function (derepression of miRNA targets) in cells will be apparent to one of ordinary skill in the art.

In some embodiments, a miRNA as described herein comprises a sequence listed in Table 4 of PCT Publication No. WO2020014209, incorporated herein by reference. Also incorporated herein by reference are the listing of exemplary miRNA sequences from WO2020014209.

In some embodiments, it is advantageous to silence one or more components of a gene modifying system (e.g., mRNA encoding a gene modifying polypeptide, a gene modifying Template RNA, or a heterologous object sequence expressed from the genome after successful gene modifying) in a portion of cells. In some embodiments, it is advantageous to restrict expression of a component of a gene modifying system to select cell types within a tissue of interest.

For example, it is known that in a given tissue, e.g., liver, macrophages and immune cells, e.g., Kupffer cells in the liver, may engage in uptake of a delivery vehicle for one or more components of a gene modifying system. In some embodiments, at least one binding site for at least one miRNA highly expressed in macrophages and immune cells, e.g., Kupffer cells, is included in at least one component of a gene modifying system, e.g., nucleic acid encoding a gene modifying polypeptide or a transgene. In some embodiments, a miRNA that targets the one or more binding sites is listed in a table referenced herein, e.g., miR-142, e.g., mature miRNA hsa-miR-142-5p or hsa-miR-142-3p.

In some embodiments, there may be a benefit to decreasing gene modifying polypeptide levels and/or gene modifying activity in cells in which gene modifying polypeptide expression or overexpression of a transgene may have a toxic effect. For example, it has been shown that delivery of a transgene overexpression cassette to dorsal root ganglion neurons may result in toxicity of a gene therapy (see Hordeaux et al Sci Transl Med 12 (569): eaba9188 (2020), incorporated herein by reference in its entirety). In some embodiments, at least one miRNA binding site may be incorporated into a nucleic acid component of a gene modifying system to reduce expression of a system component in a neuron, e.g., a dorsal root ganglion neuron. In some embodiments, the at least one miRNA binding site incorporated into a nucleic acid component of a gene modifying system to reduce expression of a system component in a neuron is a binding site of miR-182, e.g., mature miRNA hsa-miR-182-5p or hsa-miR-182-3p. In some embodiments, the at least one miRNA binding site incorporated into a nucleic acid component of a gene modifying system to reduce expression of a system component in a neuron is a binding site of miR-183, e.g., mature miRNA hsa-miR-183-5p or hsa-miR-183-3p. In some embodiments, combinations of miRNA binding sites may be used to enhance the restriction of expression of one or more components of a gene modifying system to a tissue or cell type of interest.

Table A5 below provides exemplary miRNAs and corresponding expressing cells, e.g., a miRNA for which one can, in some embodiments, incorporate binding sites (complementary sequences) in the transgene or polypeptide nucleic acid, e.g., to decrease expression in that off-target cell.

TABLE A5
Exemplary miRNA from off-target cells and tissues

| Silenced cell | miRNA | | | SEQ ID |
|---|---|---|---|---|
| type | name | Mature miRNA | miRNA sequence | NO: |
| Kupffer cells | miR-142 | hsa-miR-142-5p | cauaaaguagaaagcacuacu | 37663 |
| Kupffer cells | miR-142 | hsa-miR-142-3p | uguaguguuuccuacuuuaugga | 37664 |
| Dorsal root | miR-182 | hsa-miR-182-5p | uuuggcaaugguagaacucacacu | 37665 |
| ganglion neurons | | | | |
| Dorsal root | miR-182 | hsa-miR-182-3p | ugguucuagacuugccaacua | 37666 |
| ganglion neurons | | | | |
| Dorsal root | miR-183 | hsa-miR-183-5p | uauggcacugguagaauucacu | 37667 |
| ganglion neurons | | | | |
| Table XDorsal | miR-183 | hsa-miR-183-3p | gugaauuaccgaagggccauaa | 37668 |
| root ganglion | | | | |
| neurons | | | | |
| Hepatocytes | miR-122 | hsa-miR-122-5p | uggagugugacaaugguguuug | 37669 |
| Hepatocytes | miR-122 | hsa-miR-122-3p | aacgccauuaucacacuaaaua | 37670 |

In some embodiments, a gene modifying system, template RNA, or polypeptide described herein is administered to or is active in (e.g., is more active in) a target tissue, e.g., a first tissue. In some embodiments, the gene modifying system, template RNA, or polypeptide is not administered to or is less active in (e.g., not active in) a non-target tissue. In some embodiments, a gene modifying system, template RNA, or polypeptide described herein is useful for modifying DNA in a target tissue, e.g., a first tissue, (e.g., and not modifying DNA in a non-target tissue).

In some embodiments, a gene modifying system comprises (a) a polypeptide described herein or a nucleic acid encoding the same, (b) a template nucleic acid (e.g., template RNA) described herein, and (c) one or more first tissue-specific expression-control sequences specific to the target tissue, wherein the one or more first tissue-specific expression-control sequences specific to the target tissue are in operative association with (a), (b), or (a) and (b), wherein, when associated with (a), (a) comprises a nucleic acid encoding the polypeptide.

In some embodiments, the nucleic acid in (b) comprises RNA.

In some embodiments, the nucleic acid in (b) comprises DNA.

In some embodiments, the nucleic acid in (b): (i) is single-stranded or comprises a single-stranded segment, e.g., is single-stranded DNA or comprises a single-stranded segment and one or more double stranded segments; (ii) has inverted terminal repeats; or (iii) both (i) and (ii).

In some embodiments, the nucleic acid in (b) is double-stranded or comprises a double-stranded segment.

In some embodiments, (a) comprises a nucleic acid encoding the polypeptide.

In some embodiments, the nucleic acid in (a) comprises RNA.

In some embodiments, the nucleic acid in (a) comprises DNA.

In some embodiments, the nucleic acid in (a): (i) is single-stranded or comprises a single-stranded segment, e.g., is single-stranded DNA or comprises a single-stranded segment and one or more double stranded segments; (ii) has inverted terminal repeats; or (iii) both (i) and (ii).

In some embodiments, the nucleic acid in (a) is double-stranded or comprises a double-stranded segment.

In some embodiments, the nucleic acid in (a), (b), or (a) and (b) is linear.

In some embodiments, the nucleic acid in (a), (b), or (a) and (b) is circular, e.g., a plasmid or minicircle.

In some embodiments, the heterologous object sequence is in operative association with a first promoter.

In some embodiments, the one or more first tissue-specific expression-control sequences comprises a tissue specific promoter.

In some embodiments, the tissue-specific promoter comprises a first promoter in operative association with: (i) the heterologous object sequence, (ii) a nucleic acid encoding the retroviral RT, or (iii) (i) and (ii).

In some embodiments, the one or more first tissue-specific expression-control sequences comprises a tissue-specific microRNA recognition sequence in operative association with: (i) the heterologous object sequence, (ii) a nucleic acid encoding the retroviral RT domain, or (iii) (i) and (ii).

In some embodiments, a system comprises a tissue-specific promoter, and the system further comprises one or more tissue-specific microRNA recognition sequences, wherein: (i) the tissue specific promoter is in operative association with: (I) the heterologous object sequence, (II) a nucleic acid encoding the retroviral RT domain, or (III) (I) and (II); and/or (ii) the one or more tissue-specific microRNA recognition sequences are in operative association with: (I) the heterologous object sequence, (II) a nucleic acid encoding the retroviral RT, or (III) (I) and (II).

In some embodiments, wherein (a) comprises a nucleic acid encoding the polypeptide, the nucleic acid comprises a promoter in operative association with the nucleic acid encoding the polypeptide.

In some embodiments, the nucleic acid encoding the polypeptide comprises one or more second tissue-specific expression-control sequences specific to the target tissue in operative association with the polypeptide coding sequence.

In some embodiments, the one or more second tissue-specific expression-control sequences comprises a tissue specific promoter.

In some embodiments, the tissue-specific promoter is the promoter in operative association with the nucleic acid encoding the polypeptide.

In some embodiments, the one or more second tissue-specific expression-control sequences comprises a tissue-specific microRNA recognition sequence.

In some embodiments, the promoter in operative association with the nucleic acid encoding the polypeptide is a tissue-specific promoter, the system further comprising one or more tissue-specific microRNA recognition sequences.

In some embodiments, a nucleic acid component of a system provided by the invention is a sequence (e.g., encoding the polypeptide or comprising a heterologous object sequence) flanked by untranslated regions (UTRs) that modify protein expression levels. Various 5′ and 3′ UTRs can affect protein expression. For example, in some embodiments, the coding sequence may be preceded by a 5′ UTR that modifies RNA stability or protein translation. In some embodiments, the sequence may be followed by a 3′ UTR that modifies RNA stability or translation. In some embodiments, the sequence may be preceded by a 5′ UTR and followed by a 3′ UTR that modify RNA stability or translation. In some embodiments, the 5′ and/or 3′ UTR may be selected from the 5′ and 3′ UTRs of complement factor 3 (C3) (CACTCCTCCCCATCCTCTCCCTCTGTCCCTCTGTCCCTCTGACCCTGCACTGTCCCAG CACC; SEQ ID NO: 11,004) or orosomucoid 1 (ORM1) (CAGGACACAGCCTTGGATCAGGACAGAGACTTGGGGGCCATCCTGCCCCTCCAACC CGACATGTGTACCTCAGCTTTTTCCCTCACTTGCATCAATAAAGCTTCTGTGTTTGGA ACAGCTAA; SEQ ID NO: 11,005) (Asrani et al. RNA Biology 2018). In certain embodiments, the 5′ UTR is the 5′ UTR from C3 and the 3′ UTR is the 3′ UTR from ORM1. In certain embodiments, a 5′ UTR and 3′ UTR for protein expression, e.g., mRNA (or DNA encoding the RNA) for a heterologous gene modifying polypeptide or heterologous object sequence, comprise optimized expression sequences. In some embodiments, the 5′ UTR comprises GGGAAAUAAGAGAGAAAAGAAGAGUAAGAAGAAAUAUAAGAGCCACC (SEQ ID NO: 11,006) and/or the 3′ UTR comprising UGAUAAUAGGCUGGAGCCUCGGUGGCCAUGCUUCUUGCCCCUUGGGCCUCCCCCC AGCCCCUCCUCCCCUUCCUGCACCCGUACCCCCGUGGUCUUUGAAUAAAGUCUGA (SEQ ID NO: 11,007), e.g., as described in Richner et al. Cell 168 (6): P1114-1125 (2017), the sequences of which are incorporated herein by reference. In some embodiments, a 5′ and/or 3′ UTR may be selected to enhance protein expression. In some embodiments, a 5′ and/or 3′ UTR may be selected to modify protein expression such that overproduction inhibition is minimized. In some embodiments, UTRs are around a coding sequence, e.g., outside the coding sequence and in other embodiments proximal to the coding sequence, In some embodiments, additional regulatory elements (e.g., miRNA binding sites, cis-regulatory sites) are included in the UTRs.

In some embodiments, an open reading frame of a gene modifying system, e.g., an ORF of an mRNA (or DNA encoding an mRNA) encoding a gene modifying polypeptide or one or more ORFs of an mRNA (or DNA encoding an mRNA) of a heterologous object sequence, is flanked by a 5′ and/or 3′ untranslated region (UTR) that enhances the expression thereof. In some embodiments, the 5′ UTR of an mRNA component (or transcript produced from a DNA component) of the system comprises the sequence 5′-GGGAAAUAAGAGAGAAAAGAAGAGUAAGAAGAAAUAUAAGAGCCACC-3′; SEQ ID NO: 11,008). In some embodiments, the 3′ UTR of an mRNA component (or transcript produced from a DNA component) of the system comprises the sequence 5′-UGAUAAUAGGCUGGAGCCUCGGUGGCCAUGCUUCUUGCCCCUUGGGCCUCCCCCC AGCCCCUCCUCCCCUUCCUGCACCCGUACCCCCGUGGUCUUUGAAUAAAGUCUGA-3′ (SEQ ID NO: 11,009). This combination of 5′ UTR and 3′ UTR has been shown to result in desirable expression of an operably linked ORF by Richner et al. Cell 168 (6): P1114-1125 (2017), the teachings and sequences of which are incorporated herein by reference. In some embodiments, a system described herein comprises a DNA encoding a transcript, wherein the DNA comprises the corresponding 5′ UTR and 3′ UTR sequences, with T substituting for U in the above-listed sequence). In some embodiments, a DNA vector used to produce an RNA component of the system further comprises a promoter upstream of the 5′ UTR for initiating in vitro transcription, e.g, a T7, T3, or SP6 promoter. The 5′ UTR above begins with GGG, which is a suitable start for optimizing transcription using T7 RNA polymerase. For tuning transcription levels and altering the transcription start site nucleotides to fit alternative 5′ UTRs, the teachings of Davidson et al. Pac Symp Biocomput 433-443 (2010) describe T7 promoter variants, and the methods of discovery thereof, that fulfill both of these traits.

### Viral Vectors and Components Thereof

Viruses are a useful source of delivery vehicles for the systems described herein, in addition to a source of relevant enzymes or domains as described herein, e.g., as sources of polymerases and polymerase functions used herein, e.g., DNA-dependent DNA polymerase, RNA-dependent RNA polymerase, RNA-dependent DNA polymerase, DNA-dependent RNA polymerase, reverse transcriptase. Some enzymes, e.g., reverse transcriptases, may have multiple activities, e.g., be capable of both RNA-dependent DNA polymerization and DNA-dependent DNA polymerization, e.g., first and second strand synthesis. In some embodiments, the virus used as a gene modifying delivery system or a source of components thereof may be selected from a group as described by Baltimore Bacteriol Rev 35 (3): 235-241 (1971).

In some embodiments, the virus is selected from a Group I virus, e.g., is a DNA virus and packages dsDNA into virions. In some embodiments, the Group I virus is selected from, e.g., Adenoviruses, Herpesviruses, Poxviruses.

In some embodiments, the virus is selected from a Group II virus, e.g., is a DNA virus and packages ssDNA into virions. In some embodiments, the Group II virus is selected from, e.g., Parvoviruses. In some embodiments, the parvovirus is a dependoparvovirus, e.g., an adeno-associated virus (AAV).

In some embodiments, the virus is selected from a Group III virus, e.g., is an RNA virus and packages dsRNA into virions. In some embodiments, the Group III virus is selected from, e.g., Reoviruses. In some embodiments, one or both strands of the dsRNA contained in such virions is a coding molecule able to serve directly as mRNA upon transduction into a host cell, e.g., can be directly translated into protein upon transduction into a host cell without requiring any intervening nucleic acid replication or polymerization steps.

In some embodiments, the virus is selected from a Group IV virus, e.g., is an RNA virus and packages ssRNA(+) into virions. In some embodiments, the Group IV virus is selected from, e.g., Coronaviruses, Picornaviruses, Togaviruses. In some embodiments, the ssRNA(+) contained in such virions is a coding molecule able to serve directly as mRNA upon transduction into a host cell, e.g., can be directly translated into protein upon transduction into a host cell without requiring any intervening nucleic acid replication or polymerization steps.

In some embodiments, the virus is selected from a Group V virus, e.g., is an RNA virus and packages ssRNA(−) into virions. In some embodiments, the Group V virus is selected from, e.g., Orthomyxoviruses, Rhabdoviruses. In some embodiments, an RNA virus with an ssRNA(−) genome also carries an enzyme inside the virion that is transduced to host cells with the viral genome, e.g., an RNA-dependent RNA polymerase, capable of copying the ssRNA(−) into ssRNA(+) that can be translated directly by the host.

In some embodiments, the virus is selected from a Group VI virus, e.g., is a retrovirus and packages ssRNA(+) into virions. In some embodiments, the Group VI virus is selected from, e.g., retroviruses. In some embodiments, the retrovirus is a lentivirus, e.g., HIV-1, HIV-2, SIV, BIV. In some embodiments, the retrovirus is a spumavirus, e.g., a foamy virus, e.g., HFV, SFV, BFV. In some embodiments, the ssRNA(+) contained in such virions is a coding molecule able to serve directly as mRNA upon transduction into a host cell, e.g., can be directly translated into protein upon transduction into a host cell without requiring any intervening nucleic acid replication or polymerization steps. In some embodiments, the ssRNA(+) is first reverse transcribed and copied to generate a dsDNA genome intermediate from which mRNA can be transcribed in the host cell. In some embodiments, an RNA virus with an ssRNA(+) genome also carries an enzyme inside the virion that is transduced to host cells with the viral genome, e.g., an RNA-dependent DNA polymerase, capable of copying the ssRNA(+) into dsDNA that can be transcribed into mRNA and translated by the host. In some embodiments, the reverse transcriptase from a Group VI retrovirus is incorporated as the reverse transcriptase domain of a gene modifying polypeptide.

In some embodiments, the virus is selected from a Group VII virus, e.g., is a retrovirus and packages dsRNA into virions. In some embodiments, the Group VII virus is selected from, e.g., Hepadnaviruses. In some embodiments, one or both strands of the dsRNA contained in such virions is a coding molecule able to serve directly as mRNA upon transduction into a host cell, e.g., can be directly translated into protein upon transduction into a host cell without requiring any intervening nucleic acid replication or polymerization steps. In some embodiments, one or both strands of the dsRNA contained in such virions is first reverse transcribed and copied to generate a dsDNA genome intermediate from which mRNA can be transcribed in the host cell. In some embodiments, an RNA virus with a dsRNA genome also carries an enzyme inside the virion that is transduced to host cells with the viral genome, e.g., an RNA-dependent DNA polymerase, capable of copying the dsRNA into dsDNA that can be transcribed into mRNA and translated by the host. In some embodiments, the reverse transcriptase from a Group VII retrovirus is incorporated as the reverse transcriptase domain of a gene modifying polypeptide.

In some embodiments, virions used to deliver nucleic acid in this invention may also carry enzymes involved in the process of gene modification. For example, a retroviral virion may contain a reverse transcriptase domain that is delivered into a host cell along with the nucleic acid. In some embodiments, an RNA template may be associated with a gene modifying polypeptide within a virion, such that both are co-delivered to a target cell upon transduction of the nucleic acid from the viral particle. In some embodiments, the nucleic acid in a virion may comprise DNA, e.g., linear ssDNA, linear dsDNA, circular ssDNA, circular dsDNA, minicircle DNA, dbDNA, ceDNA. In some embodiments, the nucleic acid in a virion may comprise RNA, e.g., linear ssRNA, linear dsRNA, circular ssRNA, circular dsRNA. In some embodiments, a viral genome may circularize upon transduction into a host cell, e.g., a linear ssRNA molecule may undergo a covalent linkage to form a circular ssRNA, a linear dsRNA molecule may undergo a covalent linkage to form a circular dsRNA or one or more circular ssRNA. In some embodiments, a viral genome may replicate by rolling circle replication in a host cell. In some embodiments, a viral genome may comprise a single nucleic acid molecule, e.g., comprise a non-segmented genome. In some embodiments, a viral genome may comprise two or more nucleic acid molecules, e.g., comprise a segmented genome. In some embodiments, a nucleic acid in a virion may be associated with one or proteins. In some embodiments, one or more proteins in a virion may be delivered to a host cell upon transduction. In some embodiments, a natural virus may be adapted for nucleic acid delivery by the addition of virion packaging signals to the target nucleic acid, wherein a host cell is used to package the target nucleic acid containing the packaging signals.

In some embodiments, a virion used as a delivery vehicle may comprise a commensal human virus. In some embodiments, a virion used as a delivery vehicle may comprise an anellovirus, the use of which is described in WO2018232017A1, which is incorporated herein by reference in its entirety.

### AAV Administration

In some embodiments, an adeno-associated virus (AAV) is used in conjunction with the system, template nucleic acid, and/or polypeptide described herein. In some embodiments, an AAV is used to deliver, administer, or package the system, template nucleic acid, and/or polypeptide described herein. In some embodiments, the AAV is a recombinant AAV (rAAV).

In some embodiments, a system comprises (a) a polypeptide described herein or a nucleic acid encoding the same, (b) a template nucleic acid (e.g., template RNA) described herein, and (c) one or more first tissue-specific expression-control sequences specific to the target tissue, wherein the one or more first tissue-specific expression-control sequences specific to the target tissue are in operative association with (a), (b), or (a) and (b), wherein, when associated with (a), (a) comprises a nucleic acid encoding the polypeptide.

In some embodiments, a system described herein further comprises a first recombinant adeno-associated virus (rAAV) capsid protein; wherein the at least one of (a) or (b) is associated with the first rAAV capsid protein, wherein at least one of (a) or (b) is flanked by AAV inverted terminal repeats (ITRs).

In some embodiments, (a) and (b) are associated with the first rAAV capsid protein.

In some embodiments, (a) and (b) are on a single nucleic acid.

In some embodiments, the system further comprises a second rAAV capsid protein, wherein at least one of (a) or (b) is associated with the second rAAV capsid protein, and wherein the at least one of (a) or (b) associated with the second rAAV capsid protein is different from the at least one of (a) or (b) is associated with the first rAAV capsid protein.

In some embodiments, the at least one of (a) or (b) is associated with the first or second rAAV capsid protein is dispersed in the interior of the first or second rAAV capsid protein, which first or second rAAV capsid protein is in the form of an AAV capsid particle.

In some embodiments, the system further comprises a nanoparticle, wherein the nanoparticle is associated with at least one of (a) or (b).

In some embodiments, (a) and (b), respectively are associated with: a) a first rAAV capsid protein and a second rAAV capsid protein; b) a nanoparticle and a first rAAV capsid protein; c) a first rAAV capsid protein; d) a first adenovirus capsid protein; e) a first nanoparticle and a second nanoparticle; or f) a first nanoparticle.

Viral vectors are useful for delivering all or part of a system provided by the invention, e.g., for use in methods provided by the invention. Systems derived from different viruses have been employed for the delivery of polypeptides or nucleic acids; for example: integrase-deficient lentivirus, adenovirus, adeno-associated virus (AAV), herpes simplex virus, and baculovirus (reviewed in Hodge et al. Hum Gene Ther 2017; Narayanavari et al. Crit Rev Biochem Mol Biol 2017; Boehme et al. Curr Gene Ther 2015).

Adenoviruses are common viruses that have been used as gene delivery vehicles given well-defined biology, genetic stability, high transduction efficiency, and ease of large-scale production (see, for example, review by Lee et al. Genes & Diseases 2017). They possess linear dsDNA genomes and come in a variety of serotypes that differ in tissue and cell tropisms. In order to prevent replication of infectious virus in recipient cells, adenovirus genomes used for packaging are deleted of some or all endogenous viral proteins, which are provided in trans in viral production cells. This renders the genomes helper-dependent, meaning they can only be replicated and packaged into viral particles in the presence of the missing components provided by so-called helper functions. A helper-dependent adenovirus system with all viral ORFs removed may be compatible with packaging foreign DNA of up to ~37 kb (Parks et al. J Virol 1997). In some embodiments, an adenoviral vector is used to deliver DNA corresponding to the polypeptide or template component of the gene modifying system, or both are contained on separate or the same adenoviral vector. In some embodiments, the adenovirus is a helper-dependent adenovirus (HD-AdV) that is incapable of self-packaging. In some embodiments, the adenovirus is a high-capacity adenovirus (HC-AdV) that has had all or a substantial portion of endogenous viral ORFs deleted, while retaining the necessary sequence components for packaging into adenoviral particles. For this type of vector, the only adenoviral sequences required for genome packaging are noncoding sequences: the inverted terminal repeats (ITRs) at both ends and the packaging signal at the 5′-end (Jager et al. Nat Protoc 2009). In some embodiments, the adenoviral genome also comprises stuffer DNA to meet a minimal genome size for optimal production and stability (see, for example, Hausl et al. Mol Ther 2010). In some embodiments, an adenovirus is used to deliver a gene modifying system to the liver.

In some embodiments, an adenovirus is used to deliver a gene modifying system to HSCs, e.g., HDAd5/35++. HDAd5/35++ is an adenovirus with modified serotype 35 fibers that de-target the vector from the liver (Wang et al. Blood Adv 2019). In some embodiments, the adenovirus that delivers a gene modifying system to HSCs utilizes a receptor that is expressed specifically on primitive HSCs, e.g., CD46.

Adeno-associated viruses (AAV) belong to the parvoviridae family and more specifically constitute the dependoparvovirus genus. The AAV genome is composed of a linear single-stranded DNA molecule which contains approximately 4.7 kilobases (kb) and consists of two major open reading frames (ORFs) encoding the non-structural Rep (replication) and structural Cap (capsid) proteins. A second ORF within the cap gene was identified that encodes the assembly-activating protein (AAP). The DNAs flanking the AAV coding regions are two cis-acting inverted terminal repeat (ITR) sequences, approximately 145 nucleotides in length, with interrupted palindromic sequences that can be folded into energetically stable hairpin structures that function as primers of DNA replication. In addition to their role in DNA replication, the ITR sequences have been shown to be involved in viral DNA integration into the cellular genome, rescue from the host genome or plasmid, and encapsidation of viral nucleic acid into mature virions (Muzyczka, (1992) Curr. Top. Micro. Immunol. 158:97-129). In some embodiments, one or more gene modifying nucleic acid components is flanked by ITRs derived from AAV for viral packaging. See, e.g., WO2019113310.

In some embodiments, one or more components of the gene modifying system are carried via at least one AAV vector. In some embodiments, the at least one AAV vector is selected for tropism to a particular cell, tissue, organism. In some embodiments, the AAV vector is pseudotyped, e.g., AAV2/8, wherein AAV2 describes the design of the construct but the capsid protein is replaced by that from AAV8. It is understood that any of the described vectors could be pseudotype derivatives, wherein the capsid protein used to package the AAV genome is derived from that of a different AAV serotype. Without wishing to be limited in vector choice, a list of exemplary AAV serotypes can be found in Table 18. In some embodiments, an AAV to be employed for gene modifying may be evolved for novel cell or tissue tropism as has been demonstrated in the literature (e.g., Davidsson et al. Proc Natl Acad Sci USA 2019).

In some embodiments, the AAV delivery vector is a vector which has two AAV inverted terminal repeats (ITRs) and a nucleotide sequence of interest (for example, a sequence coding for a gene modifying polypeptide or a DNA template, or both), each of said ITRs having an interrupted (or noncontiguous) palindromic sequence, i.e., a sequence composed of three segments: a first segment and a last segment that are identical when read 5′->3′ but hybridize when placed against each other, and a segment that is different that separates the identical segments. See, for example, WO2012123430.

Conventionally, AAV virions with capsids are produced by introducing a plasmid or plasmids encoding the rAAV or scAAV genome, Rep proteins, and Cap proteins (Grimm et al, 1998). Upon introduction of these helper plasmids in trans, the AAV genome is “rescued” (i.e., released and subsequently recovered) from the host genome, and is further encapsidated to produce infectious AAV. In some embodiments, one or more gene modifying nucleic acids are packaged into AAV particles by introducing the ITR-flanked nucleic acids into a packaging cell in conjunction with the helper functions.

In some embodiments, the AAV genome is a so called self-complementary genome (referred to as scAAV), such that the sequence located between the ITRs contains both the desired nucleic acid sequence (e.g., DNA encoding the gene modifying polypeptide or template, or both) in addition to the reverse complement of the desired nucleic acid sequence, such that these two components can fold over and self-hybridize. In some embodiments, the self-complementary modules are separated by an intervening sequence that permits the DNA to fold back on itself, e.g., forms a stem-loop. An scAAV has the advantage of being poised for transcription upon entering the nucleus, rather than being first dependent on ITR priming and second-strand synthesis to form dsDNA. In some embodiments, one or more gene modifying components is designed as an scAAV, wherein the sequence between the AAV ITRs contains two reverse complementing modules that can self-hybridize to create dsDNA.

In some embodiments, nucleic acid (e.g., encoding a polypeptide, or a template, or both) delivered to cells is closed-ended, linear duplex DNA (CELID DNA or ceDNA). In some embodiments, ceDNA is derived from the replicative form of the AAV genome (Li et al. PLOS One 2013). In some embodiments, the nucleic acid (e.g., encoding a polypeptide, or a template DNA, or both) is flanked by ITRs, e.g., AAV ITRs, wherein at least one of the ITRs comprises a terminal resolution site and a replication protein binding site (sometimes referred to as a replicative protein binding site). In some embodiments, the ITRs are derived from an adeno-associated virus, e.g., AAV1, AAV2, AAV3, AAV4, AAV5, AAV6, AAV7, AAV8, AAV9, AAV10, AAV11, AAV12, or a combination thereof. In some embodiments, the ITRs are symmetric. In some embodiments, the ITRs are asymmetric. In some embodiments, at least one Rep protein is provided to enable replication of the construct. In some embodiments, the at least one Rep protein is derived from an adeno-associated virus, e.g., AAV1, AAV2, AAV3, AAV4, AAV5, AAV6, AAV7, AAV8, AAV9, AAV10, AAV11, AAV12, or a combination thereof. In some embodiments, ceDNA is generated by providing a production cell with (i) DNA flanked by ITRs, e.g., AAV ITRs, and (ii) components required for ITR-dependent replication, e.g., AAV proteins Rep78 and Rep52 (or nucleic acid encoding the proteins). In some embodiments, ceDNA is free of any capsid protein, e.g., is not packaged into an infectious AAV particle. In some embodiments, ceDNA is formulated into LNPs (see, for example, WO2019051289A1).

In some embodiments, the ceDNA vector consists of two self-complementary sequences, e.g., asymmetrical or symmetrical or substantially symmetrical ITRs as defined herein, flanking said expression cassette, wherein the ceDNA vector is not associated with a capsid protein. In some embodiments, the ceDNA vector comprises two self-complementary sequences found in an AAV genome, where at least one ITR comprises an operative Rep-binding element (RBE) (also sometimes referred to herein as “RBS”) and a terminal resolution site (trs) of AAV or a functional variant of the RBE. See, for example, WO2019113310.

In some embodiments, the AAV genome comprises two genes that encode four replication proteins and three capsid proteins, respectively. In some embodiments, the genes are flanked on either side by 145-bp inverted terminal repeats (ITRs). In some embodiments, the virion comprises up to three capsid proteins (Vp1, Vp2, and/or Vp3), e.g., produced in a 1:1:10 ratio. In some embodiments, the capsid proteins are produced from the same open reading frame and/or from differential splicing (Vp1) and alternative translational start sites (Vp2 and Vp3, respectively). Generally, Vp3 is the most abundant subunit in the virion and participates in receptor recognition at the cell surface defining the tropism of the virus. In some embodiments, Vp1 comprises a phospholipase domain, e.g., which functions in viral infectivity, in the N-terminus of Vp1.

In some embodiments, packaging capacity of the viral vectors limits the size of the gene modifying system that can be packaged into the vector. For example, the packaging capacity of the AAVs can be about 4.5 kb (e.g., about 3.0, 3.5, 4.0, 4.5, 5.0, 5.5, or 6.0 kb), e.g., including one or two inverted terminal repeats (ITRs), e.g., 145 base ITRs.

In some embodiments, recombinant AAV (rAAV) comprises cis-acting 145-bp ITRs flanking vector transgene cassettes, e.g., providing up to 4.5 kb for packaging of foreign DNA. Subsequent to infection, rAAV can, in some instances, express a fusion protein of the invention and persist without integration into the host genome by existing episomally in circular head-to-tail concatemers. rAAV can be used, for example, in vitro and in vivo. In some embodiments, AAV-mediated gene delivery requires that the length of the coding sequence of the gene is equal or greater in size than the wild-type AAV genome.

AAV delivery of genes that exceed this size and/or the use of large physiological regulatory elements can be accomplished, for example, by dividing the protein(s) to be delivered into two or more fragments. In some embodiments, the N-terminal fragment is fused to an intein-N sequence. In some embodiments, the C-terminal fragment is fused to an intein-C sequence. In embodiments, the fragments are packaged into two or more AAV vectors.

In some embodiments, dual AAV vectors are generated by splitting a large transgene expression cassette in two separate halves (5′ and 3′ ends, or head and tail), e.g., wherein each half of the cassette is packaged in a single AAV vector (of <5 kb). The re-assembly of the full-length transgene expression cassette can, in some embodiments, then be achieved upon co-infection of the same cell by both dual AAV vectors. In some embodiments, co-infection is followed by one or more of: (1) homologous recombination (HR) between 5′ and 3′ genomes (dual AAV overlapping vectors); (2) ITR-mediated tail-to-head concatemerization of 5′ and 3′ genomes (dual AAV trans-splicing vectors); and/or (3) a combination of these two mechanisms (dual AAV hybrid vectors). In some embodiments, the use of dual AAV vectors in vivo results in the expression of full-length proteins. In some embodiments, the use of the dual AAV vector platform represents an efficient and viable gene transfer strategy for transgenes of greater than about 4.0, 4.1, 4.2, 4.3, 4.4, 4.5, 4.6, 4.7, 4.8, 4.9, or 5.0 kb in size. In some embodiments, AAV vectors can also be used to transduce cells with target nucleic acids, e.g., in the in vitro production of nucleic acids and peptides. In some embodiments, AAV vectors can be used for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994); each of which is incorporated herein by reference in their entirety). The construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989) (incorporated by reference herein in their entirety).

In some embodiments, a gene modifying polypeptide described herein (e.g., with or without one or more guide nucleic acids) can be delivered using AAV, lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as described in U.S. Pat. No. 8,454,972 and as in clinical trials involving AAV. For adenovirus, the route of administration, formulation and dose can be as described in U.S. Pat. No. 8,404,658 and as in clinical trials involving adenovirus. For plasmid delivery, the route of administration, formulation and dose can be as described in U.S. Pat. No. 5,846,946 and as in clinical studies involving plasmids. Doses can be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. In some embodiments, the viral vectors can be injected into the tissue of interest. For cell-type specific gene modifying, the expression of the gene modifying polypeptide and optional guide nucleic acid can, in some embodiments, be driven by a cell-type specific promoter.

In some embodiments, AAV allows for low toxicity, for example, due to the purification method not requiring ultracentrifugation of cell particles that can activate the immune response. In some embodiments, AAV allows low probability of causing insertional mutagenesis, for example, because it does not substantially integrate into the host genome.

In some embodiments, AAV has a packaging limit of about 4.4, 4.5, 4.6, 4.7, or 4.75 kb. In some embodiments, a gene modifying polypeptide-encoding sequence, promoter, and transcription terminator can fit into a single viral vector. SpCas9 (4.1 kb) may, in some instances, be difficult to package into AAV. Therefore, in some embodiments, a gene modifying polypeptide coding sequence is used that is shorter in length than other gene modifying polypeptide coding sequences or base editors. In some embodiments, the gene modifying polypeptide encoding sequences are less than about 4.5 kb, 4.4 kb, 4.3 kb, 4.2 kb, 4.1 kb, 4 kb, 3.9 kb, 3.8 kb, 3.7 kb, 3.6 kb, 3.5 kb, 3.4 kb, 3.3 kb, 3.2 kb, 3.1 kb, 3 kb, 2.9 kb, 2.8 kb, 2.7 kb, 2.6 kb, 2.5 kb, 2 kb, or 1.5 kb.

An AAV can be AAV1, AAV2, AAV5 or any combination thereof. In some embodiments, the type of AAV is selected with respect to the cells to be targeted; e.g., AAV serotypes 1, 2, 5 or a hybrid capsid AAV1, AAV2, AAV5 or any combination thereof can be selected for targeting brain or neuronal cells; or AAV4 can be selected for targeting cardiac tissue. In some embodiments, AAV8 is selected for delivery to the liver. Exemplary AAV serotypes as to these cells are described, for example, in Grimm, D. et al, J. Virol. 82:5887-5911 (2008) (incorporated herein by reference in its entirety). In some embodiments, AAV refers all serotypes, subtypes, and naturally-occurring AAV as well as recombinant AAV. AAV may be used to refer to the virus itself or a derivative thereof. In some embodiments, AAV includes AAV1, AAV2, AAV3, AAV3B, AAV4, AAV5, AAV6, AAV6.2, AAV7, AAVrh.64Rl, AAVhu.37, AAVrh.8, AAVrh.32.33, AAV8, AAV9, AAV-DJ, AAV2/8, AAVrhIO, AAVLK03, AV10, AAV11, AAV 12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. Additional exemplary AAV serotypes are listed in Table 18.

TABLE 18
Exemplary AAV serotypes.

| Target | | |
|---|---|---|
| Tissue | Vehicle | Reference |
| Liver | AAV (AAV8¹, AAVrh.8¹, | 1. Wang et al., Mol. Ther. 18, |
| | AAVhu.37¹, AAV2/8, | 118-25 (2010) |
| | AAV2/rh10², AAV9, AAV², | 2. Ginn et al., JHEP Reports, |
| | NP40³, NP59^{2,3}, AAV3B⁵, | 100065 (2019) |
| | AAV-DJ⁴, AAV-LK01⁴, | |
| | AAV-LK02⁴, AAV-LK03⁴, | 3. Paulk et al., Mol. Ther. 26, |
| | AAV-LK19⁴, AAV5⁷ | 289-303 (2018). |
| | Adenovirus (Ad5, HC-AdV⁶) | 4. L. Lisowski et al., Nature. |
| | | 506, 382-6 (2014). |
| | | 5. L. Wang et al., Mol. Ther. |
| | | 23, 1877-87 (2015). |
| | | 6. Hausl Mol Ther (2010) |
| | | 7. Davidoff et al., Mol. Ther. |
| | | 11, 875-88 (2005) |
| Lung | AAV (AAV4, AAV5, | 1. Duncan et al., Mol Ther |
| | AAV6¹, AAV9, H22²) | Methods Clin Dev (2018) |
| | Adenovirus (Ad5, Ad3, | 2. Cooney et al., Am J Respir |
| | Ad21, Ad14)³ | Cell Mol Biol (2019) |
| | | 3. Li et al., Mol Ther Methods |
| | | Clin Dev (2019) |
| Skin | AAV (AAV6¹, AAV-LK19²) | 1. Petek et al., Mol. Ther. |
| | | (2010) |
| | | 2. L. Lisowski et al., Nature. |
| | | 506, 382-6 (2014). |
| HSCs | Adenovirus (HDAd5/35⁺⁺) | Wang et al. Blood Adv (2019) |

In some embodiments, a pharmaceutical composition (e.g., comprising an AAV as described herein) has less than 10% empty capsids, less than 8% empty capsids, less than 7% empty capsids, less than 5% empty capsids, less than 3% empty capsids, or less than 1% empty capsids. In some embodiments, the pharmaceutical composition has less than about 5% empty capsids. In some embodiments, the number of empty capsids is below the limit of detection. In some embodiments, it is advantageous for the pharmaceutical composition to have low amounts of empty capsids, e.g., because empty capsids may generate an adverse response (e.g., immune response, inflammatory response, liver response, and/or cardiac response), e.g., with little or no substantial therapeutic benefit.

In some embodiments, the residual host cell protein (rHCP) in the pharmaceutical composition is less than or equal to 100 ng/ml rHCP per 1×10¹³ vg/ml, e.g., less than or equal to 40 ng/ml rHCP per 1×10¹³ vg/ml or 1-50 ng/ml rHCP per 1×10¹³ vg/ml. In some embodiments, the pharmaceutical composition comprises less than 10 ng rHCP per 1.0×10¹³ vg, or less than 5 ng rHCP per 1.0×10¹³ vg, less than 4 ng rHCP per 1.0×10¹³ vg, or less than 3 ng rHCP per 1.0×10¹³ vg, or any concentration in between. In some embodiments, the residual host cell DNA (hcDNA) in the pharmaceutical composition is less than or equal to 5×10⁶ μg/ml hcDNA per 1×10¹³ vg/ml, less than or equal to 1.2×10⁶ μg/ml hcDNA per 1×10¹³ vg/ml, or 1×105 μg/ml hcDNA per 1×10¹³ vg/ml. In some embodiments, the residual host cell DNA in said pharmaceutical composition is less than 5.0×10⁵ μg per 1×10¹³ vg, less than 2.0×10⁵ μg per 1.0×10¹³ vg, less than 1.1×10⁵ μg per 1.0×10¹³ vg, less than 1.0×10⁵ μg hcDNA per 1.0×10¹³ vg, less than 0.9×10⁵ μg hcDNA per 1.0×10¹³ vg, less than 0.8×10⁵ μg hcDNA per 1.0×10¹³ vg, or any concentration in between.

In some embodiments, the residual plasmid DNA in the pharmaceutical composition is less than or equal to 1.7×10⁵ μg/ml per 1.0×10¹³ vg/ml, or 1×10⁵ μg/ml per 1×1.0×10¹³ vg/ml, or 1.7×10⁶ μg/ml per 1.0×10¹³ vg/ml. In some embodiments, the residual DNA plasmid in the pharmaceutical composition is less than 10.0×10⁵ μg by 1.0×10¹³ vg, less than 8.0×10⁵ μg by 1.0×10¹³ vg or less than 6.8×10⁵ μg by 1.0×10¹³ vg. In embodiments, the pharmaceutical composition comprises less than 0.5 ng per 1.0×10¹³ vg, less than 0.3 ng per 1.0×10¹³ vg, less than 0.22 ng per 1.0×10¹³ vg or less than 0.2 ng per 1.0×10¹³ vg or any intermediate concentration of bovine serum albumin (BSA). In embodiments, the benzonase in the pharmaceutical composition is less than 0.2 ng by 1.0×10¹³ vg, less than 0.1 ng by 1.0×10¹³ vg, less than 0.09 ng by 1.0×10¹³ vg, less than 0.08 ng by 1.0×10¹³ vg or any intermediate concentration. In embodiments, Poloxamer 188 in the pharmaceutical composition is about 10 to 150 ppm, about 15 to 100 ppm or about 20 to 80 ppm. In embodiments, the cesium in the pharmaceutical composition is less than 50 μg/g (ppm), less than 30 μg/g (ppm) or less than 20 pg/g (ppm) or any intermediate concentration.

In embodiments, the pharmaceutical composition comprises total impurities, e.g., as determined by SDS-PAGE, of less than 10%, less than 8%, less than 7%, less than 6%, less than 5%, less than 4%, less than 3%, less than 2%, or any percentage in between. In embodiments, the total purity, e.g., as determined by SDS-PAGE, is greater than 90%, greater than 92%, greater than 93%, greater than 94%, greater than 95%, greater than 96%, greater than 97%, greater than 98%, or any percentage in between. In embodiments, no single unnamed related impurity, e.g., as measured by SDS-PAGE, is greater than 5%, greater than 4%, greater than 3% or greater than 2%, or any percentage in between. In embodiments, the pharmaceutical composition comprises a percentage of filled capsids relative to total capsids (e.g., peak 1+peak 2 as measured by analytical ultracentrifugation) of greater than 85%, greater than 86%, greater than 87%, greater than 88%, greater than 89%, greater than 90%, greater than 91%, greater than 91.9%, greater than 92%, greater than 93%, or any percentage in between. In embodiments of the pharmaceutical composition, the percentage of filled capsids measured in peak 1 by analytical ultracentrifugation is 20-80%, 25-75%, 30-75%, 35-75%, or 37.4-70.3%. In embodiments of the pharmaceutical composition, the percentage of filled capsids measured in peak 2 by analytical ultracentrifugation is 20-80%, 20-70%, 22-65%, 24-62%, or 24.9-60.1%.

In one embodiment, the pharmaceutical composition comprises a genomic titer of 1.0 to 5.0×10¹³ vg/mL, 1.2 to 3.0×10¹³ vg/mL or 1.7 to 2.3×10¹³ vg/ml. In one embodiment, the pharmaceutical composition exhibits a biological load of less than 5 CFU/mL, less than 4 CFU/mL, less than 3 CFU/mL, less than 2 CFU/mL or less than 1 CFU/mL or any intermediate contraction. In embodiments, the amount of endotoxin according to USP, for example, USP<85> (incorporated by reference in its entirety) is less than 1.0 EU/mL, less than 0.8 EU/mL or less than 0.75 EU/mL. In embodiments, the osmolarity of a pharmaceutical composition according to USP, for example, USP<785> (incorporated by reference in its entirety) is 350 to 450 mOsm/kg, 370 to 440 mOsm/kg or 390 to 430 mOsm/kg. In embodiments, the pharmaceutical composition contains less than 1200 particles that are greater than 25 μm per container, less than 1000 particles that are greater than 25 μm per container, less than 500 particles that are greater than 25 μm per container or any intermediate value. In embodiments, the pharmaceutical composition contains less than 10,000 particles that are greater than 10 μm per container, less than 8000 particles that are greater than 10 μm per container or less than 600 particles that are greater than 10 μm per container.

In one embodiment, the pharmaceutical composition has a genomic titer of 0.5 to 5.0×10¹³ vg/mL, 1.0 to 4.0×10¹³ vg/mL, 1.5 to 3.0×10¹³ vg/ml or 1.7 to 2.3×10¹³ vg/ml. In one embodiment, the pharmaceutical composition described herein comprises one or more of the following: less than about 0.09 ng benzonase per 1.0×10¹³ vg, less than about 30 μg/g (ppm) of cesium, about 20 to 80 ppm Poloxamer 188, less than about 0.22 ng BSA per 1.0×10¹³ vg, less than about 6.8×10⁵ μg of residual DNA plasmid per 1.0×10¹³ vg, less than about 1.1×5 μg of residual hcDNA per 1.0×10¹³ vg, less than about 4 ng of rHCP per 1.0×10¹³ vg, pH 7.7 to 8.3, about 390 to 430 mOsm/kg, less than about 600 particles that are >25 μm in size per container, less than about 6000 particles that are >10 μm in size per container, about 1.7×10¹³-2.3×10¹³ vg/mL genomic titer, infectious titer of about 3.9×10⁸ to 8.4×10¹⁰ IU per 1.0×10¹³ vg, total protein of about 100-300 μg per 1.0×10¹³ vg, mean survival of >24 days in A7SMA mice with about 7.5×10¹³ vg/kg dose of viral vector, about 70 to 130% relative potency based on an in vitro cell based assay and/or less than about 5% empty capsid. In various embodiments, the pharmaceutical compositions described herein comprise any of the viral particles discussed here, retain a potency of between ±20%, between ±15%, between ±10% or within ±5% of a reference standard. In some embodiments, potency is measured using a suitable in vitro cell assay or in vivo animal model.

Additional methods of preparation, characterization, and dosing AAV particles are taught in WO2019094253, which is incorporated herein by reference in its entirety.

Additional rAAV constructs that can be employed consonant with the invention include those described in Wang et al 2019, available at://doi.org/10.1038/s41573-019-0012-9, including Table 1 thereof, which is incorporated by reference in its entirety.

### Lipid Nanoparticles

The methods and systems provided herein may employ any suitable carrier or delivery modality, including, in certain embodiments, lipid nanoparticles (LNPs). Lipid nanoparticles, in some embodiments, comprise one or more ionic lipids, such as non-cationic lipids (e.g., neutral or anionic, or zwitterionic lipids); one or more conjugated lipids (such as PEG-conjugated lipids or lipids conjugated to polymers described in Table 5 of WO2019217941; incorporated herein by reference in its entirety); one or more sterols (e.g., cholesterol); and, optionally, one or more targeting molecules (e.g., conjugated receptors, receptor ligands, antibodies); or combinations of the foregoing.

Lipids that can be used in nanoparticle formations (e.g., lipid nanoparticles) include, for example those described in Table 4 of WO2019217941, which is incorporated by reference e.g., a lipid-containing nanoparticle can comprise one or more of the lipids in Table 4 of WO2019217941. Lipid nanoparticles can include additional elements, such as polymers, such as the polymers described in Table 5 of WO2019217941, incorporated by reference.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy) propyl-1-0-(w-methoxy (polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al (2020), dx.doi.org/10.1021/acs.nanolett.0c01386, incorporated herein by reference.

In some embodiments, the lipid particle comprises an ionizable lipid, a non-cationic lipid, a conjugated lipid that inhibits aggregation of particles, and a sterol. The amounts of these components can be varied independently and to achieve desired properties. For example, in some embodiments, the lipid nanoparticle comprises an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid (e.g., encoding the gene modifying polypeptide or template nucleic acid) can be varied as desired. For example, the total lipid to nucleic acid (mass or weight) ratio can be from about 10:1 to about 30:1.

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle comprises a cationic lipid in formulation with one or more of neutral lipids, ionizable amine-containing lipids, biodegradable alkyn lipids, steroids, phospholipids including polyunsaturated lipids, structural lipids (e.g., sterols), PEG, cholesterol and polymer conjugated lipids. In some embodiments, the cationic lipid may be an ionizable cationic lipid. An exemplary cationic lipid as disclosed herein may have an effective pKa over 6.0. In embodiments, a lipid nanoparticle may comprise a second cationic lipid having a different effective pKa (e.g., greater than the first effective pKa), than the first cationic lipid. A lipid nanoparticle may comprise between 40 and 60 mol percent of a cationic lipid, a neutral lipid, a steroid, a polymer conjugated lipid, and a therapeutic agent, e.g., a nucleic acid (e.g., RNA) described herein (e.g., a template nucleic acid or a nucleic acid encoding a gene modifying polypeptide), encapsulated within or associated with the lipid nanoparticle. In some embodiments, the nucleic acid is co-formulated with the cationic lipid. The nucleic acid may be adsorbed to the surface of an LNP, e.g., an LNP comprising a cationic lipid. In some embodiments, the nucleic acid may be encapsulated in an LNP, e.g., an LNP comprising a cationic lipid. In some embodiments, the lipid nanoparticle may comprise a targeting moiety, e.g., coated with a targeting agent. In embodiments, the LNP formulation is biodegradable. In some embodiments, a lipid nanoparticle comprising one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of an RNA molecule, e.g., template RNA and/or a mRNA encoding the gene modifying polypeptide.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of WO2018/081480; I-5 or I-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-013 or 503-013 of Whitehead et al; TS-P4C2 of U.S. Pat. No. 9,708,628; I of WO2020/106946; I of WO2020/106946.

In some embodiments, the ionizable lipid is MC3 (6Z,9Z,28Z,3 lZ)-heptatriaconta-6,9,28,3 l-tetraen-19-yl-4-(dimethylamino)butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (13Z,16Z)-A,A-dimethyl-3-nonyldocosa-13, 16-dien-1-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety). In some embodiments, the ionizable lipid is heptadecan-9-yl 8-((2-hydroxyethyl) (6-oxo-6-(undecyloxy) hexyl)amino) octanoate (SM-102); e.g., as described in Example 1 of U.S. Pat. No. 9,867,888 (incorporated by reference herein in its entirety). In some embodiments, the ionizable lipid is 9Z, 12Z)-3-((4,4-bis(octyloxy) butanoyl)oxy)-2-((((3-(diethylamino) propoxy) carbonyl)oxy)methyl) propyl octadeca-9,12-dienoate (LP01) e.g., as synthesized in Example 13 of WO2015/095340 (incorporated by reference herein in its entirety). In some embodiments, the ionizable lipid is Di((Z)-non-2-en-1-yl) 9-((4-dimethylamino)butanoyl)oxy) heptadecanedioate (L319), e.g. as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl) (2-hydroxydodecyl)amino)ethyl) piperazin-1-yl)ethyl) azanediyl)bis(dodecan-2-ol) (C12-200), e.g., as synthesized in Examples 14 and 16 of WO2010/053572 (incorporated by reference herein in its entirety). In some embodiments, the ionizable lipid is; Imidazole cholesterol ester (ICE) lipid (3S, 10R, 13R, 17R)-10,13-dimethyl-17-((R)-6-methylheptan-2-yl)-2, 3, 4, 7, 8, 9, 10, 11, 12, 13, 14, 15, 16, 17-tetradecahydro-lH-cyclopenta[a]phenanthren-3-yl 3-(1H-imidazol-4-yl) propanoate, e.g., Structure (I) from WO2020/106946 (incorporated by reference herein in its entirety).

Some non-limiting examples of lipid compounds that may be used (e.g., in combination with other lipid components) to form lipid nanoparticles for the delivery of compositions described herein, e.g., nucleic acid (e.g., RNA) described herein (e.g., a template nucleic acid or a nucleic acid encoding a gene modifying polypeptide) includes,

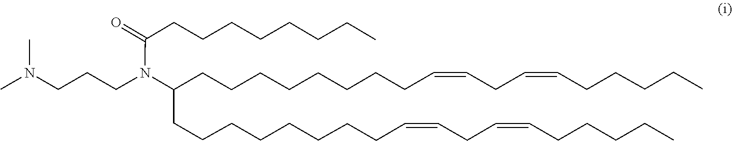 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP comprising Formula (i) is used to deliver a gene modifying composition described herein to the liver and/or hepatocyte cells.

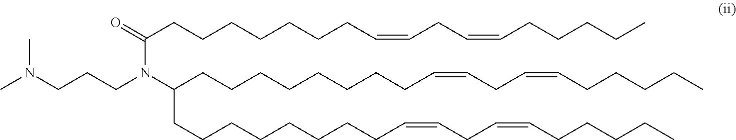 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC]

In some embodiments an LNP comprising Formula (ii) is used to deliver a gene modifying composition described herein to the liver and/or hepatocyte cells.

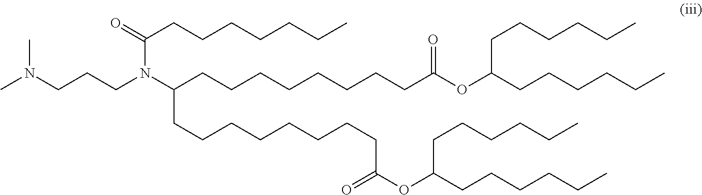 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments an LNP comprising Formula (iii) is used to deliver a gene modifying composition described herein to the liver and/or hepatocyte cells.

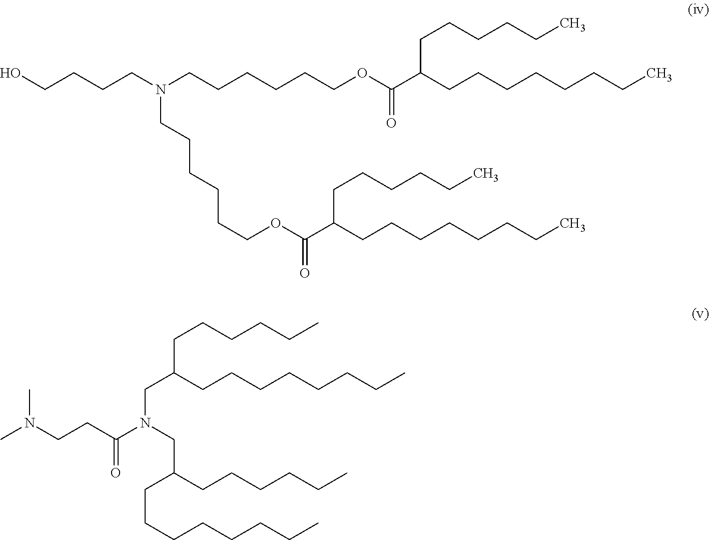

In some embodiments an LNP comprising Formula (v) is used to deliver a gene modifying composition described herein to the liver and/or hepatocyte cells.

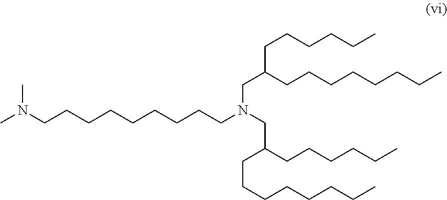 [SMILES: CCCCCCCCC(CCCCCC)CN(CCCCCCCCCN(C)C)CC(CCCCCC)CCCCCCCC]

In some embodiments an LNP comprising Formula (vi) is used to deliver a gene modifying composition described herein to the liver and/or hepatocyte cells.

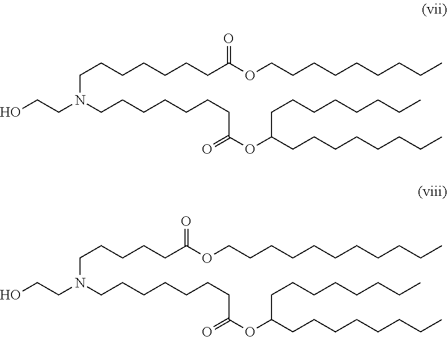 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In some embodiments an LNP comprising Formula (viii) is used to deliver a gene modifying composition described herein to the liver and/or hepatocyte cells.

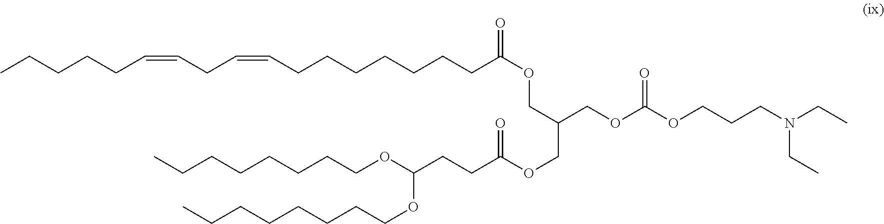 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments an LNP comprising Formula (ix) is used to deliver a gene modifying composition described herein to the liver and/or hepatocyte cells.

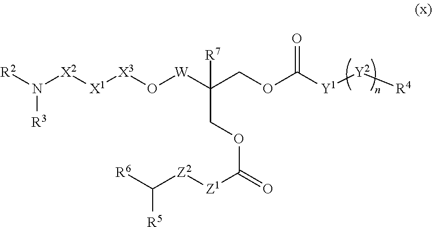

wherein
X¹ is O, NR¹, or a direct bond, X² is C2-5 alkylene, X³ is C(═O) or a direct bond, R¹ is H or Me, R³ is Ci-3 alkyl, R² is Ci-3 alkyl, or R² taken together with the nitrogen atom to which it is attached and 1-3 carbon atoms of X² form a 4-, 5-, or 6-membered ring, or X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring, Y¹ is C2-12 alkylene, Y² is selected from

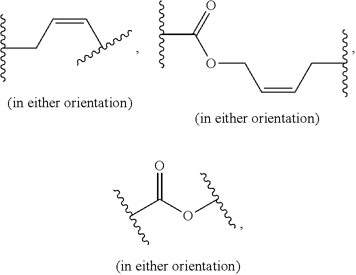 [SMILES: */C=C\C* ; *C/C=C\COC(*)=O ; *OC(*)=O]

n is 0 to 3, R⁴ is Ci-15 alkyl, Z¹ is Ci-6 alkylene or a direct bond,
Z2 is

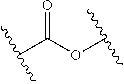 [SMILES: *OC(*)=O]

(in either orientation) or absent, provided that if Z¹ is a direct bond, Z² is absent;
R⁵ is C5-9 alkyl or C6-10 alkoxy, R⁶ is C5-9 alkyl or C6-10 alkoxy, W is methylene or a direct bond, and R⁷ is H or Me, or a salt thereof, provided that if R³ and R² are C2 alkyls, X¹ is O, X² is linear C3 alkylene, X³ is C(=0), Y¹ is linear Ce alkylene, (Y²)n-R⁴ is

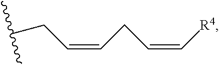

R⁴ is linear C5 alkyl, Z¹ is C2 alkylene, Z² is absent, Wis methylene, and R⁷ is H, then R⁵ and R⁶ are not Cx alkoxy.

In some embodiments an LNP comprising Formula (xii) is used to deliver a gene modifying composition described herein to the liver and/or hepatocyte cells.

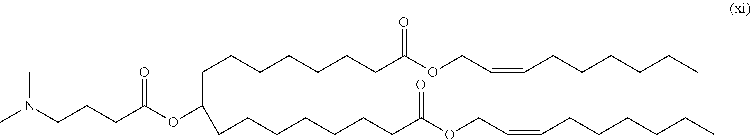 [SMILES: CCCCCC/C=C\COC(=O)CCCCCCCC(CCCCCCCC(=O)OC/C=C\CCCCCC)OC(=O)CCCN(C)C]

In some embodiments an LNP comprising Formula (xi) is used to deliver a gene modifying composition described herein to the liver and/or hepatocyte cells.

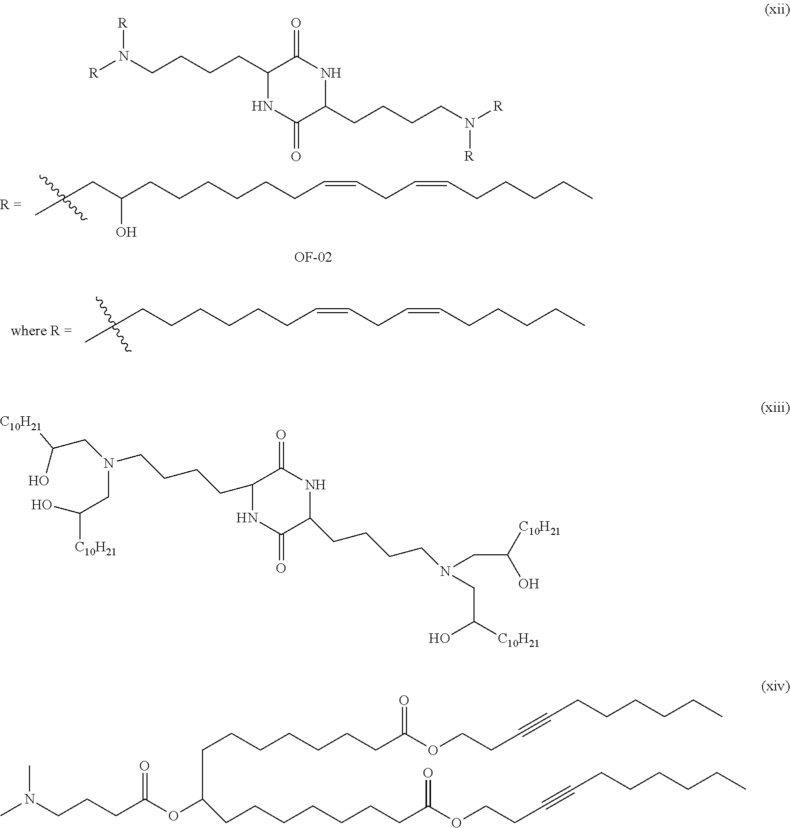

In some embodiments an LNP comprises a compound of Formula (xiii) and a compound of Formula (xiv).

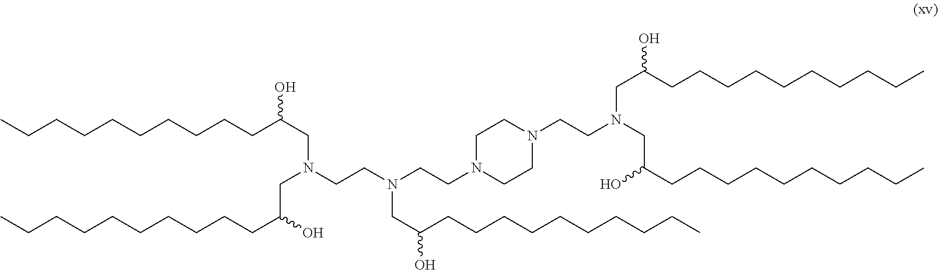 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments an LNP comprising Formula (xv) is used to deliver a gene modifying composition described herein to the liver and/or hepatocyte cells.

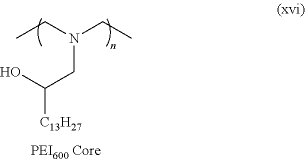

In some embodiments an LNP comprising a formulation of Formula (xvi) is used to deliver a gene modifying composition described herein to the lung endothelial cells.

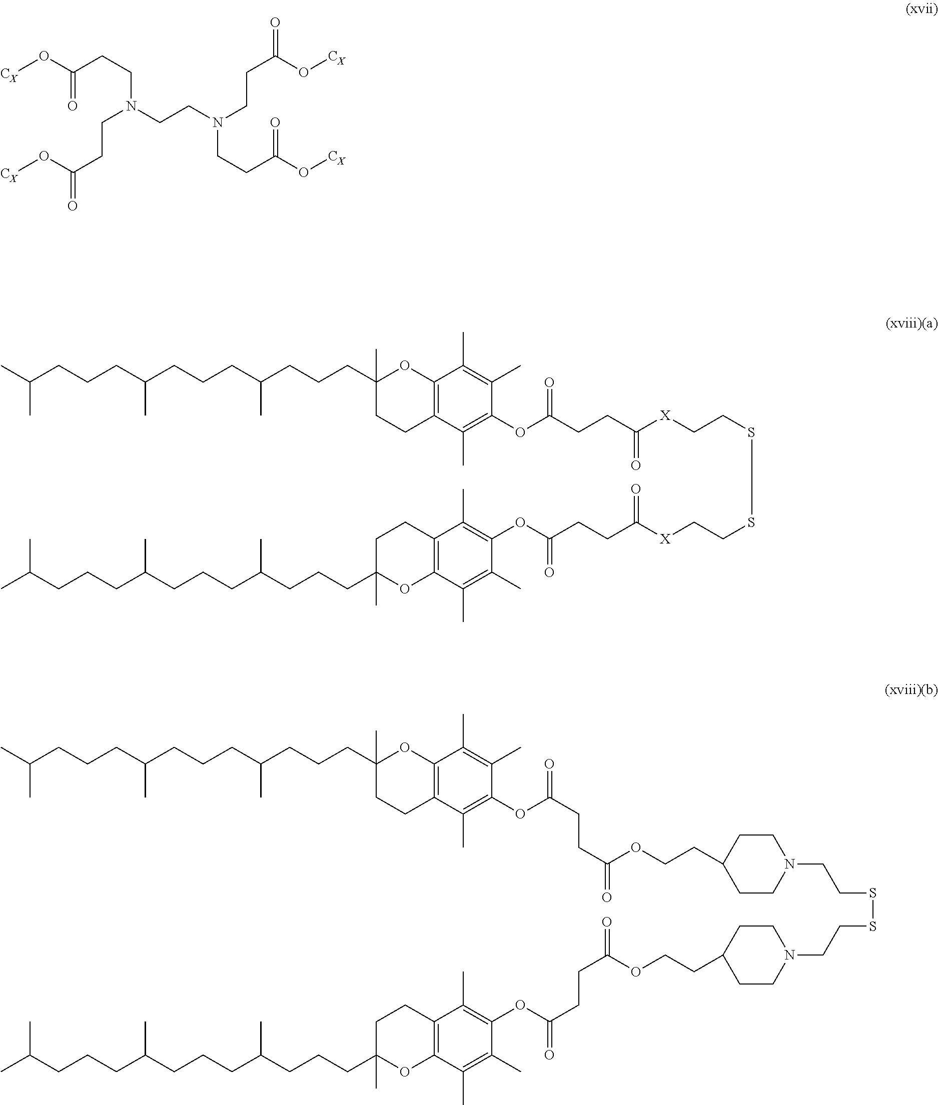

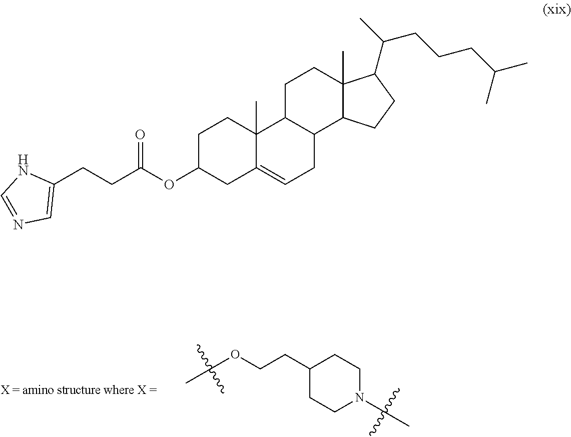 [SMILES: CC(C)CCCC(C)C1CCC2C3CC=C4CC(OC(=O)CCc5cnc[nH]5)CCC4(C)C3CCC12C ; *OCCC1CCN(*)CC1]

In some embodiments, a lipid compound used to form lipid nanoparticles for the delivery of compositions described herein, e.g., nucleic acid (e.g., RNA) described herein (e.g., a template nucleic acid or a nucleic acid encoding a gene modifying polypeptide) is made by one of the following reactions:

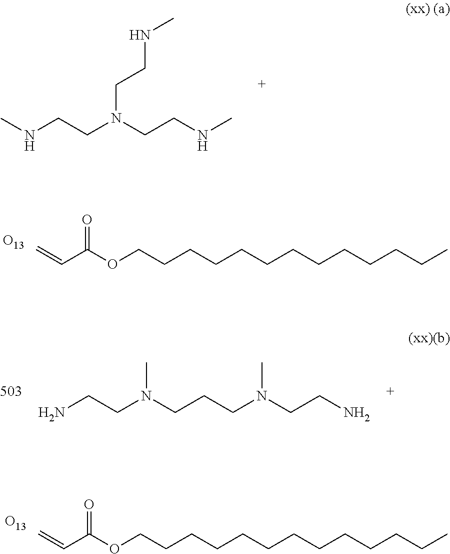

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), 1,2-dioleoyl-sn-glycero-3-phosphoethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, palmitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS). In some embodiments, the non-cationic lipid may have the following structure,

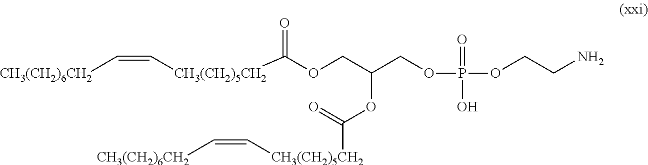

Other examples of non-cationic lipids suitable for use in the lipid nanoparticles include, without limitation, nonphosphorous lipids such as, e.g., stearylamine, dodecylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can comprise, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not comprise any phospholipids.

In some aspects, the lipid nanoparticle can further comprise a component, such as a sterol, to provide membrane integrity. One exemplary sterol that can be used in the lipid nanoparticle is cholesterol and derivatives thereof. Non-limiting examples of cholesterol derivatives include polar analogues such as 5a-cholestanol, 53-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can comprise 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), 1,2-dimyristoyl-sn-glycerol, methoxypoly ethylene glycol (DMG-PEG-2K), PEG succinate diacylglycerol (PEGS-DAG) (such as 4-0-(2′,3′-di(tetradecanoyloxy) propyl-1-0-(w-methoxy (polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the contents of all of which are incorporated herein by reference in their entirety. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3 [beta]-oxy) carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy (polyethylene glycol)-2000]. In some embodiments, the PEG-lipid comprises PEG-DMG, 1,2-dimyristoyl-sn-glycero-3-phosphoethanolamine-N-[methoxy (polyethylene glycol)-2000]. In some embodiments, the PEG-lipid comprises a structure selected from:

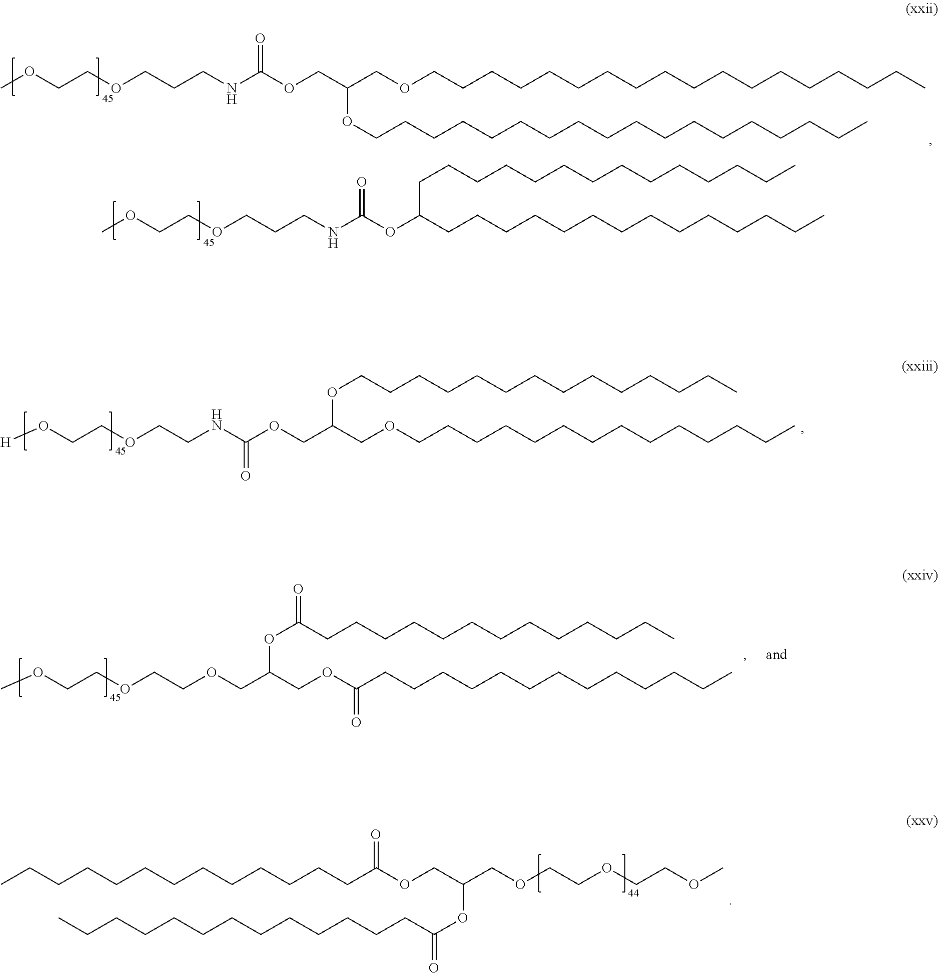

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9 and in WO2020106946A1, the contents of all of which are incorporated herein by reference in their entirety.

In some embodiments an LNP comprises a compound of Formula (xix), a compound of Formula (xxi) and a compound of Formula (xxv). In some embodiments an LNP comprising a formulation of Formula (xix), Formula (xxi) and Formula (xxv) is used to deliver a gene modifying composition described herein to the lung or pulmonary cells.

In some embodiments, a lipid nanoparticle may comprise one or more cationic lipids selected from Formula (i), Formula (ii), Formula (iii), Formula (vii), and Formula (ix). In some embodiments, the LNP may further comprise one or more neutral lipid, e.g., DSPC, DPPC, DMPC, DOPC, POPC, DOPE, SM, a steroid, e.g., cholesterol, and/or one or more polymer conjugated lipid, e.g., a pegylated lipid, e.g., PEG-DAG, PEG-PE, PEG-S-DAG, PEG-cer or a PEG dialkyoxypropylcarbamate.

In some embodiments, the PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can comprise 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition comprises 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example comprising 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation comprises ionizable lipid, phospholipid, cholesterol and a PEG-ylated lipid in a molar ratio of 50:10:38.5:1.5. In some other embodiments, the lipid particle formulation comprises ionizable lipid, cholesterol and a PEG-ylated lipid in a molar ratio of 60:38.5:1.5.

In some embodiments, the lipid particle comprises ionizable lipid, non-cationic lipid (e.g. phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle comprises ionizable lipid/non-cationic-lipid/sterol/conjugated lipid at a molar ratio of 50:10:38.5:1.5.

In an aspect, the disclosure provides a lipid nanoparticle formulation comprising phospholipids, lecithin, phosphatidylcholine and phosphatidylethanolamine.

In some embodiments, one or more additional compounds can also be included. Those compounds can be administered separately or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, a lipid nanoparticle (or a formulation comprising lipid nanoparticles) lacks reactive impurities (e.g., aldehydes or ketones), or comprises less than a preselected level of reactive impurities (e.g., aldehydes or ketones). While not wishing to be bound by theory, in some embodiments, a lipid reagent is used to make a lipid nanoparticle formulation, and the lipid reagent may comprise a contaminating reactive impurity (e.g., an aldehyde or ketone). A lipid regent may be selected for manufacturing based on having less than a preselected level of reactive impurities (e.g., aldehydes or ketones). Without wishing to be bound by theory, in some embodiments, aldehydes can cause modification and damage of RNA, e.g., cross-linking between bases and/or covalently conjugating lipid to RNA (e.g., forming lipid-RNA adducts). This may, in some instances, lead to failure of a reverse transcriptase reaction and/or incorporation of inappropriate bases, e.g., at the site(s) of lesion(s), e.g., a mutation in a newly synthesized target DNA.

In some embodiments, a lipid nanoparticle formulation is produced using a lipid reagent comprising less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content. In some embodiments, a lipid nanoparticle formulation is produced using a lipid reagent comprising less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, a lipid nanoparticle formulation is produced using a lipid reagent comprising: (i) less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content; and (ii) less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, the lipid nanoparticle formulation is produced using a plurality of lipid reagents, and each lipid reagent of the plurality independently meets one or more criterion described in this paragraph. In some embodiments, each lipid reagent of the plurality meets the same criterion, e.g., a criterion of this paragraph.

In some embodiments, the lipid nanoparticle formulation comprises less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content. In some embodiments, the lipid nanoparticle formulation comprises less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, the lipid nanoparticle formulation comprises: (i) less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content; and (ii) less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species.

In some embodiments, one or more, or optionally all, of the lipid reagents used for a lipid nanoparticle as described herein or a formulation thereof comprise less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content. In some embodiments, one or more, or optionally all, of the lipid reagents used for a lipid nanoparticle as described herein or a formulation thereof comprise less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, one or more, or optionally all, of the lipid reagents used for a lipid nanoparticle as described herein or a formulation thereof comprise: (i) less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content; and (ii) less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species.

In some embodiments, total aldehyde content and/or quantity of any single reactive impurity (e.g., aldehyde) species is determined by liquid chromatography (LC), e.g., coupled with tandem mass spectrometry (MS/MS), e.g., according to the method described in Example 40 of PCT/US21/20948. In some embodiments, reactive impurity (e.g., aldehyde) content and/or quantity of reactive impurity (e.g., aldehyde) species is determined by detecting one or more chemical modifications of a nucleic acid molecule (e.g., an RNA molecule, e.g., as described herein) associated with the presence of reactive impurities (e.g., aldehydes), e.g., in the lipid reagents. In some embodiments, reactive impurity (e.g., aldehyde) content and/or quantity of reactive impurity (e.g., aldehyde) species is determined by detecting one or more chemical modifications of a nucleotide or nucleoside (e.g., a ribonucleotide or ribonucleoside, e.g., comprised in or isolated from a template nucleic acid, e.g., as described herein) associated with the presence of reactive impurities (e.g., aldehydes), e.g., in the lipid reagents, e.g., according to the method described in Example 41 of PCT/US21/20948. In embodiments, chemical modifications of a nucleic acid molecule, nucleotide, or nucleoside are detected by determining the presence of one or more modified nucleotides or nucleosides, e.g., using LC-MS/MS analysis, e.g., according to the method described in Example 41 of PCT/US21/20948.

In some embodiments, a nucleic acid (e.g., RNA) described herein (e.g., a template nucleic acid or a nucleic acid encoding a gene modifying polypeptide) does not comprise an aldehyde modification, or comprises less than a preselected amount of aldehyde modifications. In some embodiments, on average, a nucleic acid has less than 50, 20, 10, 5, 2, or 1 aldehyde modifications per 1000 nucleotides, e.g., wherein a single cross-linking of two nucleotides is a single aldehyde modification. In some embodiments, the aldehyde modification is an RNA adduct (e.g., a lipid-RNA adduct). In some embodiments, the aldehyde-modified nucleotide is cross-linking between bases. In some embodiments, a nucleic acid (e.g., RNA) described herein comprises less than 50, 20, 10, 5, 2, or 1 cross-links between nucleotide.

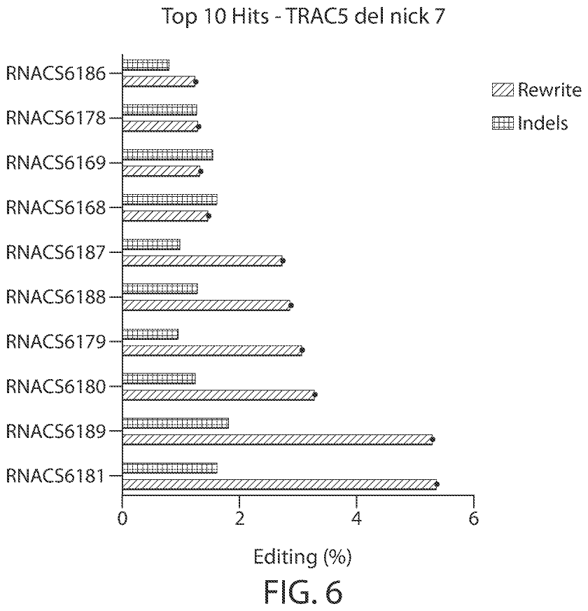

In some embodiments, LNPs are directed to specific tissues by the addition of targeting domains. For example, biological ligands may be displayed on the surface of LNPs to enhance interaction with cells displaying cognate receptors, thus driving association with and cargo delivery to tissues wherein cells express the receptor. In some embodiments, the biological ligand may be a ligand that drives delivery to the liver, e.g., LNPs that display GalNAc result in delivery of nucleic acid cargo to hepatocytes that display asialoglycoprotein receptor (ASGPR). The work of Akinc et al. Mol Ther 18 (7): 1357-1364 (2010) teaches the conjugation of a trivalent GalNAc ligand to a PEG-lipid (GalNAc-PEG-DSG) to yield LNPs dependent on ASGPR for observable LNP cargo effect (see, e.g., FIG. 6 therein). Other ligand-displaying LNP formulations, e.g., incorporating folate, transferrin, or antibodies, are discussed in WO2017223135, which is incorporated herein by reference in its entirety, in addition to the references used therein, namely Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; and Peer and Lieberman, Gene Ther. 2011 18:1127-1133.

In some embodiments, LNPs are selected for tissue-specific activity by the addition of a Selective ORgan Targeting (SORT) molecule to a formulation comprising traditional components, such as ionizable cationic lipids, amphipathic phospholipids, cholesterol and poly(ethylene glycol) (PEG) lipids. The teachings of Cheng et al. Nat Nanotechnol 15 (4): 313-320 (2020) demonstrate that the addition of a supplemental “SORT” component precisely alters the in vivo RNA delivery profile and mediates tissue-specific (e.g., lungs, liver, spleen) gene delivery and editing as a function of the percentage and biophysical property of the SORT molecule.

In some embodiments, the LNPs comprise biodegradable, ionizable lipids. In some embodiments, the LNPs comprise (9Z,12Z)-3-((4,4-bis(octyloxy) butanoyl)oxy)-2-((((3-(diethylamino) propoxy) carbonyl)oxy)methyl) propyl octadeca-9,12-dienoate, also called 3-((4,4-bis(octyloxy) butanoyl)oxy)-2-((((3-(diethylamino) propoxy) carbonyl)oxy)methyl) propyl(9Z,12Z)-octadeca-9,12-dienoate) or another ionizable lipid. See, e.g, lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, an LNP described herein comprises a lipid described in Table 19.

TABLE 19

| Exemplary | | | |
|---|---|---|---|
| Lipids | | Molecular | |
| LIPID ID | Chemical Name | Weight | Structure |
| LIPIDV003 | (9Z,12Z)-3-((4,4-bis- (octyloxy)butanoyl)- oxy)-2-((((3-(diethyl- amino)propoxy)carbon- yl)oxy)methyl)propyl octadeca-9,12-dienoate | 852.29 | 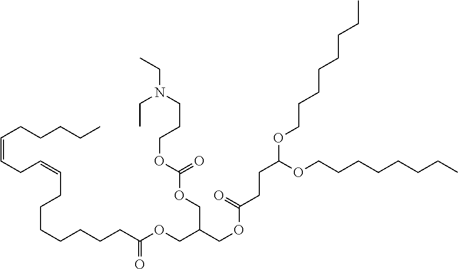 [SMILES: CCCCC/C=C\C/C=C\CCCCCCCC(=O)OCC(COC(=O)CCC(OCCCCCCCC)OCCCCCCCC)COC(=O)OCCCN(CC)CC] |
| LIPIDV004 | Heptadecan-9-yl 8-((2- hydroxyethyl)(8-(non- yloxy)-8-oxooctyl)- amino)octanoate | 710.18 | 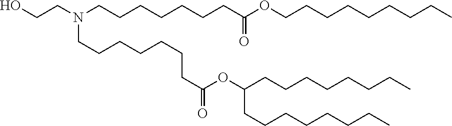 [SMILES: CCCCCCCCCOC(=O)CCCCCCCN(CCO)CCCCCCCC(=O)OC(CCCCCCCC)CCCCCCCC] |
| LIPIDV005 | | 919.56 | 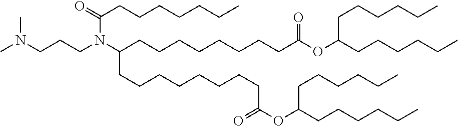 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC] |

In some embodiments, multiple components of a gene modifying system may be prepared as a single LNP formulation, e.g., an LNP formulation comprises mRNA encoding for the gene modifying polypeptide and an RNA template. Ratios of nucleic acid components may be varied in order to maximize the properties of a therapeutic. In some embodiments, the ratio of RNA template to mRNA encoding a gene modifying polypeptide is about 1:1 to 100:1, e.g., about 1:1 to 20:1, about 20:1 to 40:1, about 40:1 to 60:1, about 60:1 to 80:1, or about 80:1 to 100:1, by molar ratio. In other embodiments, a system of multiple nucleic acids may be prepared by separate formulations, e.g., one LNP formulation comprising a template RNA and a second LNP formulation comprising an mRNA encoding a gene modifying polypeptide. In some embodiments, the system may comprise more than two nucleic acid components formulated into LNPs. In some embodiments, the system may comprise a protein, e.g., a gene modifying polypeptide, and a template RNA formulated into at least one LNP formulation.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

An LNP may, in some instances, be relatively homogenous. A polydispersity index may be used to indicate the homogeneity of an LNP, e.g., the particle size distribution of the lipid nanoparticles. A small (e.g., less than 0.3) polydispersity index generally indicates a narrow particle size distribution. An LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of an LNP may be from about 0.10 to about 0.20.

The zeta potential of an LNP may be used to indicate the electrokinetic potential of the composition. In some embodiments, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of an LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a protein and/or nucleic acid, e.g., gene modifying polypeptide or mRNA encoding the polypeptide, describes the amount of protein and/or nucleic acid that is encapsulated or otherwise associated with an LNP after preparation, relative to the initial amount provided. The encapsulation efficiency is desirably high (e.g., close to 100%). The encapsulation efficiency may be measured, for example, by comparing the amount of protein or nucleic acid in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the lipid nanoparticles described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

An LNP may optionally comprise one or more coatings. In some embodiments, an LNP may be formulated in a capsule, film, or table having a coating. A capsule, film, or tablet including a composition described herein may have any useful size, tensile strength, hardness or density.

Additional exemplary lipids, formulations, methods, and characterization of LNPs are taught by WO2020061457, which is incorporated herein by reference in its entirety.

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51 (34): 8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference.

Additional specific LNP formulations useful for delivery of nucleic acids are described in U.S. Pat. Nos. 8,158,601 and 8,168,775, both incorporated by reference, which include formulations used in patisiran, sold under the name ONPATTRO.

Exemplary dosing of gene modifying LNP may include about 0.1, 0.25, 0.3, 0.5, 1, 2, 3, 4, 5, 6, 8, 10, or 100 mg/kg (RNA). Exemplary dosing of AAV comprising a nucleic acid encoding one or more components of the system may include an MOI of about 10¹¹, 10¹², 10¹³, and 10¹⁴ vg/kg.

### Kits, Articles of Manufacture, and Pharmaceutical Compositions

In an aspect the disclosure provides a kit comprising a heterologous and/or retrotransposon gene modifying polypeptide or a heterologous and/or retrotransposon gene modifying system, e.g., as described herein. In some embodiments, the kit comprises a gene modifying polypeptide (or a nucleic acid encoding the polypeptide) and a template RNA (or DNA encoding the template RNA). In some embodiments, the kit further comprises a reagent for introducing the system into a cell, e.g., transfection reagent, LNP, and the like. In some embodiments, the kit is suitable for any of the methods described herein. In some embodiments, the kit comprises one or more elements, compositions (e.g., pharmaceutical compositions), gene modifying polypeptides, and/or gene modifying systems, or a functional fragment or component thereof, e.g., disposed in an article of manufacture. In some embodiments, the kit comprises instructions for use thereof.

In an aspect, the disclosure provides an article of manufacture, e.g., in which a kit as described herein, or a component thereof, is disposed.

In an aspect, the disclosure provides a pharmaceutical composition comprising a gene modifying polypeptide or a gene modifying system, e.g., as described herein. In some embodiments, the pharmaceutical composition further comprises a pharmaceutically acceptable carrier or excipient. In some embodiments, the pharmaceutical composition comprises a template RNA and/or an RNA encoding the polypeptide. In embodiments, the pharmaceutical composition has one or more (e.g., 1, 2, 3, or 4) of the following characteristics:
(a) less than 1% (e.g., less than 0.5%, 0.4%, 0.3%, 0.2%, or 0.1%) DNA template relative to the template RNA and/or the RNA encoding the polypeptide, e.g., on a molar basis;
(b) less than 1% (e.g., less than 0.5%, 0.4%, 0.3%, 0.2%, or 0.1%) uncapped RNA relative to the template RNA and/or the RNA encoding the polypeptide, e.g., on a molar basis;
(c) less than 1% (e.g., less than 0.5%, 0.4%, 0.3%, 0.2%, or 0.1%) partial length RNAs relative to the template RNA and/or the RNA encoding the polypeptide, e.g., on a molar basis;
(d) substantially lacks unreacted cap dinucleotides.

### Chemistry, Manufacturing, and Controls (CMC)

Purification of protein therapeutics is described, for example, in Franks, Protein Biotechnology: Isolation, Characterization, and Stabilization, Humana Press (2013); and in Cutler, Protein Purification Protocols (Methods in Molecular Biology), Humana Press (2010).

In some embodiments, a gene modifying system, polypeptide, and/or template nucleic acid (e.g., template RNA) conforms to certain quality standards. In some embodiments, a gene modifying system, polypeptide, and/or template nucleic acid (e.g., template RNA) produced by a method described herein conforms to certain quality standards. Accordingly, the disclosure is directed, in some aspects, to methods of manufacturing a gene modifying system, polypeptide, and/or template nucleic acid (e.g., template RNA) that conforms to certain quality standards, e.g., in which said quality standards are assayed. The disclosure is also directed, in some aspects, to methods of assaying said quality standards in a gene modifying system, polypeptide, and/or template nucleic acid (e.g., template RNA). In some embodiments, quality standards include, but are not limited to, one or more (e.g., 1, 2, 3, 4, 5, 6, 7, 8, 9, 10, 11, or 12) of the following:
(i) the length of the template RNA, e.g., whether the template RNA has a length that is above a reference length or within a reference length range, e.g., whether at least 80%, 85%, 90%, 95%, 96%, 97%, 98%, or 99% of the template RNA present is greater than 100, 125, 150, 175, or 200 nucleotides long;
(ii) the presence, absence, and/or length of a polyA tail on the template RNA, e.g., whether at least 80%, 85%, 90%, 95%, 96%, 97%, 98%, or 99% of the template RNA present contains a poly A tail (e.g., a polyA tail that is at least 5, 10, 20, 30, 50, 70, 100 nucleotides in length (SEQ ID NO: 37640));
(iii) the presence, absence, and/or type of a 5′ cap on the template RNA, e.g., whether at least 80%, 85%, 90%, 95%, 96%, 97%, 98%, or 99% of the template RNA present contains a 5′ cap, e.g., whether that cap is a 7-methylguanosine cap, e.g., a O-Me-m7G cap;
(iv) the presence, absence, and/or type of one or more modified nucleotides (e.g., selected from pseudouridine, dihydrouridine, inosine, 7-methylguanosine, 1-N-methylpseudouridine (1-Me-Ψ), 5-methoxyuridine (5-MO-U), 5-methylcytidine (5mC), or a locked nucleotide) in the template RNA, e.g., whether at least 80%, 85%, 90%, 95%, 96%, 97%, 98%, or 99% of the template RNA present contains one or more modified nucleotides;
(v) the stability of the template RNA (e.g., over time and/or under a pre-selected condition), e.g., whether at least 80%, 85%, 90%, 95%, 96%, 97%, 98%, or 99% of the template RNA remains intact (e.g., greater than 100, 125, 150, 175, or 200 nucleotides long) after a stability test;
(vi) the potency of the template RNA in a system for modifying DNA, e.g., whether at least 1% of target sites are modified after a system comprising the template RNA is assayed for potency;
(vii) the length of the polypeptide, first polypeptide, or second polypeptide, e.g., whether the polypeptide, first polypeptide, or second polypeptide has a length that is above a reference length or within a reference length range, e.g., whether at least 80%, 85%, 90%, 95%, 96%, 97%, 98%, or 99% of the polypeptide, first polypeptide, or second polypeptide present is greater than 600, 650, 700, 750, 800, 850, 900, 950, 1000, 1050, 1100, 1150, 1200, 1250, 1300, 1350, 1400, 1450, 1500, 1600, 1700, 1800, 1900, or 2000 amino acids long (and optionally, no larger than 2500, 2000, 1500, 1400, 1300, 1200, 1100, 1000, 900, 800, 700, or 600 amino acids long);
(viii) the presence, absence, and/or type of post-translational modification on the polypeptide, first polypeptide, or second polypeptide, e.g., whether at least 80, 85, 90, 95, 96, 97, 98, or 99% of the polypeptide, first polypeptide, or second polypeptide contains phosphorylation, methylation, acetylation, myristoylation, palmitoylation, isoprenylation, glipyatyon, or lipoylation, or any combination thereof;
(ix) the presence, absence, and/or type of one or more artificial, synthetic, or non-canonical amino acids (e.g., selected from ornithine, β-alanine, GABA, 8-Aminolevulinic acid, PABA, a D-amino acid (e.g., D-alanine or D-glutamate), aminoisobutyric acid, dehydroalanine, cystathionine, lanthionine, Djenkolic acid, Diaminopimelic acid, Homoalanine, Norvaline, Norleucine, Homonorleucine, homoserine, O-methyl-homoserine and O-ethyl-homoserine, ethionine, selenocysteine, selenohomocysteine, selenomethionine, selenoethionine, tellurocysteine, or telluromethionine) in the polypeptide, first polypeptide, or second polypeptide, e.g., whether at least 80%, 85%, 90%, 95%, 96%, 97%, 98%, or 99% of the polypeptide, first polypeptide, or second polypeptide present contains one or more artificial, synthetic, or non-canonical amino acids;
(x) the stability of the polypeptide, first polypeptide, or second polypeptide (e.g., over time and/or under a pre-selected condition), e.g., whether at least 80%, 85%, 90%, 95%, 96%, 97%, 98%, or 99% of the polypeptide, first polypeptide, or second polypeptide remains intact (e.g., greater than 600, 650, 700, 750, 800, 850, 900, 950, 1000, 1050, 1100, 1150, 1200, 1250, 1300, 1350, 1400, 1450, 1500, 1600, 1700, 1800, 1900, or 2000 amino acids long (and optionally, no larger than 2500, 2000, 1500, 1400, 1300, 1200, 1100, 1000, 900, 800, 700, or 600 amino acids long)) after a stability test;
(xi) the potency of the polypeptide, first polypeptide, or second polypeptide in a system for modifying DNA, e.g., whether at least 1% of target sites are modified after a system comprising the polypeptide, first polypeptide, or second polypeptide is assayed for potency; or
(xii) the presence, absence, and/or level of one or more of a pyrogen, virus, fungus, bacterial pathogen, or host cell protein, e.g., whether the system is free or substantially free of pyrogen, virus, fungus, bacterial pathogen, or host cell protein contamination.

In some embodiments, a system or pharmaceutical composition described herein is endotoxin free.

In some embodiments, the presence, absence, and/or level of one or more of a pyrogen, virus, fungus, bacterial pathogen, and/or host cell protein is determined. In embodiments, whether the system is free or substantially free of pyrogen, virus, fungus, bacterial pathogen, and/or host cell protein contamination is determined.

In some embodiments, a pharmaceutical composition or system as described herein has one or more (e.g., 1, 2, 3, or 4) of the following characteristics:
(a) less than 1% (e.g., less than 0.5%, 0.4%, 0.3%, 0.2%, or 0.1%) DNA template relative to the template RNA and/or the RNA encoding the polypeptide, e.g., on a molar basis;
(b) less than 1% (e.g., less than 0.5%, 0.4%, 0.3%, 0.2%, or 0.1%) uncapped RNA relative to the template RNA and/or the RNA encoding the polypeptide, e.g., on a molar basis;
(c) less than 1% (e.g., less than 0.5%, 0.4%, 0.3%, 0.2%, or 0.1%) partial length RNAs relative to the template RNA and/or the RNA encoding the polypeptide, e.g., on a molar basis;
(d) substantially lacks unreacted cap dinucleotides.

### EXAMPLES

### Example 1: Design and Generation of Template RNAs for TRAC Knock Out

This example describes the generation and design of exemplary TRAC template RNAs comprising various spacers, lengths, and compositions of heterologous object sequences, and PBS sequences, as well as quantification of the activity of said template RNAs in heterologous gene modifying systems of the disclosure for knock out of human TRAC gene.

To demonstrate the use of heterologous gene modifying systems comprising exemplary TRAC template RNAs to knockout TRAC, two spacers were selected targeting exon1 of TRAC gene: TRAC4 and TRAC5. These spacers previously demonstrated good INDEL activity when cells were treated with a Cas protein and gRNAs comprising the spacers (data not shown). Template RNAs comprising the two spacers were designed to insert 5 or 7 nt sequences or introduce deletions of lengths 5 and 7 bp into TRAC. Either insertion or deletion would result in knock out of functional TRAC protein expression due to the introduction of a premature stop codon into the TRAC gene. Without wishing to be bound by theory, insertions and deletions of 5 and 7 nt were selected because DNA heteroduplexes created by heterologous gene modifying systems are thought to be more likely to evade recognition by cellular mismatch repair process and result in high gene editing levels. The sequences of the insertions were TAGTG and TAGTTGA which were designed to introduce at least one stop codon. In addition, template RNAs were designed to place the insertions or deletions in two locations in the target nucleic acid. The mutations were either introduced at the protospacer nick site, or at the PAM location associated with the protospacer. In addition to the features described above, the lengths of PBS sequences and the heterologous object sequences were also varied.

Additional TRAC template RNAs were generated that were designed to result in either of two additional insertion sequences being inserted into the TRAC target nucleic acid sequence to evaluate whether there are any insertion sequence-specific differences between template RNAs. TGATG and TAACC were two additional insertions that were also tested. Without wishing to be bound by theory, DNA mismatch repair (MMR) is thought to prefer some sequence changes over others (see, e.g., Chen et al. Cell. 2021 Oct. 28; 184 (22): 5635-5652.e29; Warren et al. Molecular Cell 26, 579-592, May 25, 2007; and Gupta et al. Nat Struct Mol Biol. 2012 January; 19 (1): 72-78).

Identifiers and sequences of exemplary template RNAs are given in Tables 1A and 2A.

### Example 2: Evaluating TRAC Editing Activity of Exemplary Template RNAs Using TRAC4 Spacer

This example describes the use of exemplary heterologous gene modifying systems containing a gene modifying polypeptide and template RNAs comprising TRAC4 spacer, varied lengths and compositions of heterologous object sequences, and PBS sequences to quantify the activity of template RNAs for knock out of human TRAC gene in primary human T cells.

In this example, a template RNA contained:
(1) a gRNA spacer;
(2) a gRNA scaffold;
(3) a heterologous object sequence; and
(4) a primer binding site (PBS) sequence.

In this example, a gene modifying polypeptide contained:
(1) an endonuclease and/or DNA binding domain;
(2) a peptide linker; and
(3) a reverse transcriptase (RT) domain.

Exemplary template RNAs used are given in Table 1A. The exemplary gene modifying polypeptide was RNAIVT338, comprising the amino acid sequence of SEQ ID NO: 17522.

MPAAKRVKLDGGDKKYSIGLDIGTNSVGWAVITDEYKVPSKKFKVLGNTDRHSIKKNLIGALLF
DSGETAEATRLKRTARRRYTRRKNRICYLQEIFSNEMAKVDDSFFHRLEESFLVEEDKKHERHP
IFGNIVDEVAYHEKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKKNGLFGNLIALS
LGLTPNFKSNFDLAEDAKLQLSKDTYDDDLDNLLAQIGDQYADLFLAAKNLSDAILLSDILRVN
TEITKAPLSASMIKRYDEHHQDLTLLKALVRQQLPEKYKEIFFDQSKNGYAGYIDGGASQEEFY
KFIKPILEKMDGTEELLVKLNREDLLRKQRTFDNGSIPHQIHLGELHAILRRQEDFYPFLKDNR
EKIEKILTFRIPYYVGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDLLFKTNRKVTVKQLK
EDYFKKIECFDSVEISGVEDRFNASLGTYHDLLKIIKDKDFLDNEENEDILEDIVLTLTLFEDR
EMIEERLKTYAHLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQNEKLYLYYLQNGRD
MYVDQELDINRLSDYDVDHIVPQSFLKDDSIDNKVLTRSDKARGKSDNVPSEEVVKKMKNYWRQ
LLNAKLITQRKFDNLTKAERGGLSELDKAGFIKRQLVETRQITKHVAQILDSRMNTKYDENDKL
IREVKVITLKSKLVSDFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPLIETNGETGEIVWDKG
RDFATVRKVLSMPQVNIVKKTEVQTGGFSKESILPKRNSDKLIARKKDWDPKKYGGFDSPTVAY
SVLVVAKVEKGKSKKLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQKQLFVEQHKHYLDEI
IEQISEFSKRVILADANLDKVLSAYNKHRDKPIREQAENIIHLFTLTNLGAPAAFKYFDTTIDR
KRYTSTKEVLDATLIHQSITGLYETRIDLSQLGGDGGAEAAAKEAAAKEAAAKEAAAKALEAEA
AAKEAAAKEAAAKEAAAKAGGTAPLEEEYRLFLEAPIQNVTLLEQWKREIPKVWAEINPPGLAS
TQAPIHVQLLSTALPVRVRQYPITLEAKRSLRETIRKFRAAGILRPVHSPWNTPLLPVRKSGTS
EYRMVQDLREVNKRVETIHPTVPNPYTLLSLLPPDRIWYSVLDLKDAFFCIPLAPESQLIFAFE
WADAEEGESGQLTWTRLPQGFKNSPTLFNEALNRDLQGFRLDHPSVSLLQYVDDLLIAADTQAA
CLSATRDLLMTLAELGYRVSGKKAQLCQEEVTYLGFKIHKGSRSLSNSRTQAILQIPVPKTKRQ
VREFLGKIGYCRLFIPGFAELAQPLYAATRPGNDPLVWGEKEEEAFQSLKLALTQPPALALPSL
DKPFQLFVEETSGAAKGVLTQALGPWKRPVAYLSKRLDPVAAGWPRCLRAIAAAALLTREASKL
TFGQDIEITSSHNLESLLRSPPDKWLTNARITQYQVLLLDPPRVRFKQTAALNPATLLPETDDT
LPIHHCLDTLDSLTSTRPDLTDQPLAQAEATLFTDGSSYIRDGKRYAGAAVVTLDSVIWAEPLP
IGTSAQKAELIALTKALEWSKDKSVNIYTDSRYAFATLHVHGMIYRERGWLTAGGKAIKNAPEI
LALLTAVWLPKRVAVMHCKGHQKDDAPTSTGNRRADEVAREVAIRPLSTQATISAGKRTADGSE
FEKRTADGSEFESPKKKAKVE

Primary human T cells (CELL161) were activated using TexMACS media containing 5% human AB serum, and cytokines IL-2, IL-15 and IL-7. The heterologous gene modifying system comprising RNAIVT338 gene modifying polypeptide, and a template RNA described above were nucleofected into activated human primary T cells. Specifically, 2 μg of RNAIVT338-encoding mRNA and 0.07 μM tgRNA were added to 25 μL buffer P3 containing 250,000 human T cells. The cells were then nucleofected using the program EO115. After nucleofection, cells were grown at 37° C., 5% CO₂ in TexMACS media supplemented with 5% human AB serum for 3 days prior to cell lysis and genomic DNA extraction. Editing of the TRAC target nucleic acid sequence was assessed using amplicon sequencing (Amp-SEQ) using primers flanking the TRAC gene. At the time of cell lysis, half the cells from each condition were also run on the flow cytometer to assess phenotypic knockout efficiency of T cells. To prepare treated cells for flow cytometry, they were collected and stained with human anti-CD3 antibody (1:100). The stained cells were analyzed on the iQue3 (Sartorius) flow cytometer. For TRAC knockout analysis, cells were gated based on FSC-H and SSC-H. Second, single cells were gated based on FSC-A and FSC-H. Finally, CD3 negative cells were gated based on FSC-H and VLI-H (BV channel). CD3 negative cells are an indirect measure of TRAC knockout since TRAC is necessary for the TCR/CD3 complex.

FIG. 2 shows a graph of editing % as assessed by Amp-SEQ for primary human T cells nucleofected with select exemplary heterologous gene modifying system containing TRAC4 template RNAs designed to produce 5 nucleotide TAGTG insertions at the nick site to knock out TRAC expression. The results show that a number of TRAC4 insertion template RNAs facilitated high editing activity, with % editing values ranging from approximately 40% to approximately 51%, while INDEL % levels were less than 2.5%. FIG. 3 shows a graph of editing % as assessed by Amp-SEQ for primary human T cells nucleofected with select exemplary heterologous gene modifying system containing TRAC4 template RNAs designed to produce 7 nucleotide deletions at the nick site to knock out TRAC expression. The results show that a number of TRAC4 deletion template RNAs facilitated editing activity, with % editing values ranging from approximately 5% to approximately 14%, while INDEL % levels were less than 1%. Insertion template RNAs designed to produce 5 nucleotide TAGTG insertions at the nick site provided significantly higher editing activity than deletion template RNA designed to produce 7 nucleotide deletions at the nick sites.

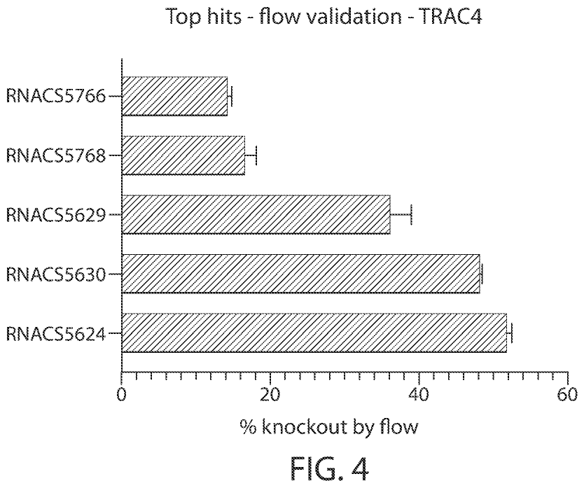

To evaluate whether the editing activities of TRAC template RNAs produces a TRAC knock out phenotype, nucleofected cells were analyzed by flow cytometry. FIG. 4 shows a graph of editing % as assessed by flow cytometry for primary human T cells nucleofected with select exemplary heterologous gene modifying system containing TRAC4 insertion and deletion template RNAs designed to produce 5 nucleotide TAGTG insertions, 7 nucleotide TAGTTGA insertions, 5 nucleotide deletions, or 7 nucleotide deletions at either the nick site or the PAM site, respectively, to knock out TRAC expression. Additionally, some insertion template RNAs were designed to insert the alternate 5 nucleotide sequences TGATG and TAACC. The results show that a number of TRAC4 template RNAs facilitated high editing activity across the variety of insertions and deletions tested, with % knock out values ranging from approximately 18% to approximately 52%. Generally, template RNAs designed to produce insertions of lengths 5 nucleotides produced a higher % of knock outs than template RNAs designed to produce insertions of length 7 nucleotides. The results show that template RNAs designed to produce insertions produced a higher % of knock outs when the insertion was introduced at the nick site as opposed to the PAM site. Generally, template RNAs designed to produce deletions did not produce as high levels of TRAC knock out as template RNAs designed to produce insertions. There was no significant difference in knock out level produced between deletion template RNAs based on the location of the deletion (at the nick site vs. at the PAM site). Several insertion template RNAs were identified that produced high editing activity and TRAC knock out phenotype. FIG. 2 shows examples of some high editing activity template RNAs designed to insert TAGTG at the nick site. For example, RNACS5630, showed ~51% editing with less than 2.5% indels. Similarly, FIG. 3 shows examples of some high editing activity template RNAs that were designed to delete 7 nucleotides from the nick site. For example, RNACS5768 showed ~14.4% editing and indels of ~0.6%. FIG. 4 shows examples, based on phenotypic knock out efficiency, of some high editing efficiency template RNAs (the two deletion template RNAs and three insertion template RNAs that resulted in the highest editing efficiencies). For example, insertion template RNA RNACS5630 showed knockout efficiency of 48% and deletion template RNA (RNACS5768) showed knockout efficiency of 16.6%.

Taken together, the results demonstrate successful precise knockout of TRAC with a number of template RNAs designed to produce insertions and deletions of varying sizes, sequences, and target locations using TRAC4 spacers. The data in FIGS. 2-4 further suggest that generally insertion lengths of less than 7 nucleotides (e.g., 5 nucleotides) may produce higher editing efficiencies than insertion lengths of 7 nucleotides or longer. The data in FIGS. 2-4 further suggest that, generally, deletion template RNAs comprising longer post-edit homology regions (e.g., 15 nucleotides or longer, e.g., 17 nucleotides) may produce higher editing efficiencies than deletion template RNAs comprising shorter post-edit homology regions (e.g., fewer than 15 nucleotides in length).

### Example 3: Evaluating TRAC Editing Activity of Exemplary Template RNAs Using TRAC5 Spacer

This example describes the use of exemplary heterologous gene modifying systems containing a gene modifying polypeptide and template RNAs comprising TRAC5 spacer, varied lengths and compositions of heterologous object sequences, and PBS sequences to quantify the activity of template RNAs for knock out of human TRAC gene in primary human T cells.

In this example, a template RNA contained:
(1) a gRNA spacer;
(2) a gRNA scaffold;
(3) a heterologous object sequence; and
(4) a primer binding site (PBS) sequence.

In this example, a gene modifying polypeptide contained:
(1) an endonuclease and/or DNA binding domain;
(2) a peptide linker; and
(3) a reverse transcriptase (RT) domain.

Exemplary template RNAs used are given in Table 1A. Nucleotide modifications are noted as follows: phosphorothioate linkages denoted by an asterisk, 2′-O-methyl groups denoted by an ‘m’ preceding a nucleotide. The exemplary gene modifying polypeptide was RNAIVT338, comprising the amino acid sequence of SEQ ID NO: 17522.

Primary human T cells (CELL161) were activated using TexMACS media containing 5% human AB serum, and cytokines IL-2, IL-15 and IL-7. The heterologous gene modifying system comprising RNAIVT338 gene modifying polypeptide, and a template RNA described above were nucleofected into activated human primary T cells. Specifically, 2 μg of RNAIVT338-encoding mRNA and 0.07 μM tgRNA were added to 25 μL buffer P3 containing 250,000 human T cells. The cells were then nucleofected using the program EO115. After nucleofection, cells were grown at 37° C., 5% CO₂ in TexMACS media supplemented with 5% human AB serum for 3 days prior to cell lysis and genomic DNA extraction. Editing of the TRAC target nucleic acid sequence was assessed using amplicon sequencing (Amp-SEQ) using primers flanking the TRAC gene. At the time of cell lysis, half the cells from each condition were run on the flow cytometer to assess phenotypic knockout efficiency of T cells as described above.

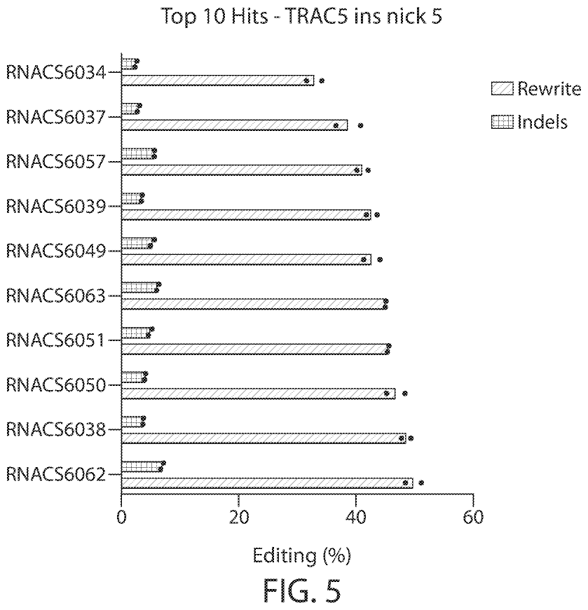

FIG. 5 shows a graph of editing % as assessed by Amp-SEQ for primary human T cells nucleofected with select exemplary heterologous gene modifying system containing TRAC5 template RNAs designed to produce 5 nucleotide TAGTG insertions at the nick site to knock out TRAC expression. The results show that a number of TRAC5 insertion template RNAs facilitated high editing activity, with % editing values ranging from approximately 35% to approximately 48%, while INDEL % levels were approximately 2-5%. FIG. 6 shows a graph of editing % as assessed by Amp-SEQ for primary human T cells nucleofected with select exemplary heterologous gene modifying system containing TRAC5 template RNAs designed to produce 7 nucleotide deletions at the nick site to knock out TRAC expression. The results show that a number of TRAC5 deletion template RNAs facilitated editing activity, with % editing values ranging from approximately 1.5% to approximately 5.5%, while INDEL % levels were less than 2%. Insertion template RNAs designed to produce 5 nucleotide TAGTG insertions at the nick site provided significantly higher editing activity than deletion template RNA designed to produce 7 nucleotide deletions at the nick sites.

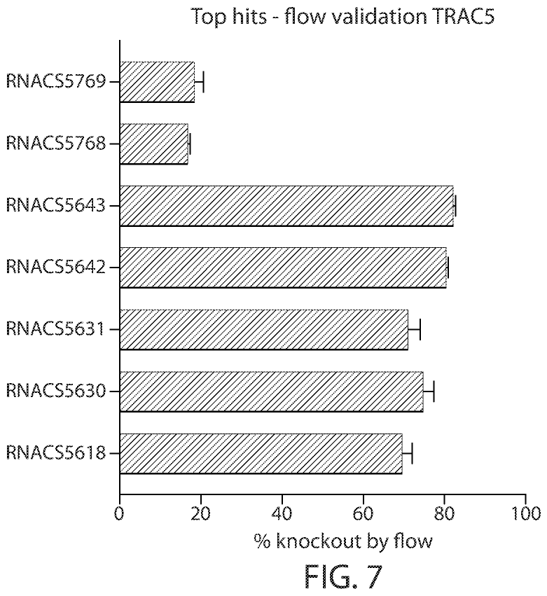

To evaluate whether the editing activities of TRAC template RNAs produce a TRAC knock out phenotype, nucleofected cells were analyzed by flow cytometry. FIG. 7 shows a graph of knock out phenotype % as assessed by flow cytometry for primary human T cells nucleofected with select exemplary heterologous gene modifying system containing TRAC5 insertion and deletion template RNAs designed to produce 5 nucleotide TAGTG insertions, 7 nucleotide TAGTTGA insertions, 5 nucleotide deletions, or 7 nucleotide deletions at either the nick site or the PAM site, respectively, to knock out TRAC expression. Additionally, some insertion template RNAs were designed to insert the alternate 5 nucleotide sequences TGATG and TAACC. The results show that a number of TRAC5 template RNAs facilitated high editing activity across the variety of insertions and deletions tested, with % knock out values ranging from approximately 15% to approximately 83%. The highest editing % achieved across all TRAC5 template RNAs as assessed by Amp-SEQ was ~50%, with indels less than 8%, using Ins5 nick P9R²² (RNACS6062). Similar to the results seen with TRAC4 template RNAs, insertion TRAC5 template RNAs produced higher % editing and higher % TRAC knock out levels than deletion TRAC5 template RNAs. Generally, template RNAs designed to produce insertions of lengths 5 nucleotides produced a higher % knock out than template RNAs designed to produce insertions of length 7 nucleotides. The results show that TRAC5 template RNAs designed to produce insertions produced a higher % of knock outs when the insertion was introduced at the nick site as opposed to the PAM site. Generally, TRAC5 template RNAs designed to produce deletions did not produce as high levels of TRAC knock out as TRAC5 template RNAs designed to produce insertions. There was no significant difference in knock out level produced between deletion TRAC5 template RNAs based on the location of the deletion (at the nick site vs. at the PAM site).

Several insertion template RNAs were identified that produce high editing activity and TRAC knock out phenotype. FIG. 5 shows examples of some high editing activity template RNA designed to insert TAGTG at the nick site. For example, RNACS6062 showed ~50% editing with less than 7% indels. Similarly, FIG. 6 shows examples of some high editing activity template RNAs that were designed to delete 7 nucleotides from the nick site. For example, RNACS6181 showed ~5.3% editing and indels of 1.6%. FIG. 7 shows examples, based on phenotypic knock out efficiency, of some high editing efficiency template RNAs. For example, insertion template RNA RNACS6062 showed knockout efficiency of 80% and deletion template RNA RNACS6181 showed knockout efficiency of ~17%.

Taken together, the results demonstrate successful precise knockout of TRAC with a number of template RNAs designed to produce insertions and deletions of varying sizes, sequences, and target locations using TRAC5 spacers.

### Example 4: Evaluating TRAC Editing Activity of Exemplary Template RNAs with Different Insert Sequences Using TRAC4 Spacer

This example describes the use of exemplary heterologous gene modifying systems containing a gene modifying polypeptide and template RNAs comprising TRAC4 spacer, varied lengths and compositions of heterologous object sequences, and PBS sequences to quantify the activity of template RNAs for knock out of human TRAC gene in primary human T cells.

In this example, a template RNA contained:
(1) a gRNA spacer;
(2) a gRNA scaffold;
(3) a heterologous object sequence; and
(4) a primer binding site (PBS) sequence.

In this example, a gene modifying polypeptide contained:
(1) an endonuclease and/or DNA binding domain;
(2) a peptide linker; and
(3) a reverse transcriptase (RT) domain.

Exemplary template RNAs used are given in Table 1A. Nucleotide modifications are noted as follows: phosphorothioate linkages denoted by an asterisk, 2′-O-methyl groups denoted by an ‘m’ preceding a nucleotide. The exemplary gene modifying polypeptide was RNAIVT338, comprising the amino acid sequence of SEQ ID NO: 17522.

Primary human T cells (CELL161) were activated using TexMACS media containing 5% human AB serum, and cytokines IL-2, IL-15 and IL-7. The heterologous gene modifying system comprising RNAIVT338 gene modifying polypeptide, and a template RNA described above were nucleofected into activated human primary T cells. Specifically, 2 μg of RNAIVT338-encoding mRNA and 0.07 μM tgRNA were added to 25 μL buffer P3 containing 250,000 human T cells. The cells were then nucleofected using the program EO115. After nucleofection, cells were grown at 37° C., 5% CO₂ in TexMACS media supplemented with 5% human AB serum for 3 days prior to cell lysis and genomic DNA extraction. Editing of the TRAC target nucleic acid sequence was assessed using amplicon sequencing (Amp-SEQ) using primers flanking the TRAC gene.

As shown in FIGS. 8A-8B, TRAC4 insertion template RNAs designed to insert any of three different 5 nucleotide insert sequences facilitated high editing activity, with % editing values ranging from approximately 35% to approximately 51%, while INDEL % levels ranging from approximately 2.5% to approximately 9%. Differences in editing efficiency were observed with different insert sequence template RNAs. TRAC4 template RNAs designed to insert TAGTG (Ins 5) and TAACC (Ins 5 v2) produced higher % editing than TRAC4 template RNAs designed to insert TGATG (Ins 5 v1). These differences highlight the influence of insertion compositions in achieving high editing efficiencies. For example, for TRAC4 template RNAs that were designed to insert TAGTG, RNACS5630 showed editing efficiency of ~51% and indel levels of ~2.4%. For TRAC4 template RNAs that were designed to insert TAACC, RNACS5962 achieved editing efficiency of ~38% and indel levels of ~2.3%. For TRAC4 template RNAs that were designed to insert TAGCC, similar editing activity was obtained compared to TRAC4 template RNAs designed to insert TAGTG, with highest editing activity achieved with RNACS6002. Editing % using “Ins 5 Nick v2 P9R¹⁶” was ~50% and indels were ~4.8%. The results showed that the exemplary heterologous gene modifying systems achieved high levels of precise insertion of three different 5 nucleotide sequences into the human TRAC gene in primary human T cells, and further showed that there were some differences in editing levels based upon the insertion sequence.

As a further evaluation of the effects of insert sequence composition on editing activity, TRAC template RNAs are designed that insert every possible 5 nucleotide sequence into the TRAC target nucleic acid sequence (see, e.g., Tables 3A and 4A). Template RNAs are designed to insert NNNNN where N can be A, T, G or C. 1024 different TRAC template RNAs comprising the different 5 nucleotide insertions are evaluated (e.g., using the methods of Examples 1-3) see, e.g., Table 4A, and various insertions sequences are identified that precisely insert a 5 nucleotide sequence into the human TRAC gene, e.g., in primary human T cells.

### Example 5: Evaluating the Dose Response of Exemplary Human Template RNAs with Different Insert Sequences in TRAC

This example describes the use of exemplary heterologous gene modifying systems containing a gene modifying polypeptide and template RNAs comprising TRAC4 and TRAC5 spacers, varied lengths and compositions of heterologous object sequences, and PBS sequences to quantify the activity of template RNAs for knock out of TRAC gene.

In this example, a template RNA contained:
(1) a gRNA spacer;
(2) a gRNA scaffold;
(3) a heterologous object sequence; and
(4) a primer binding site (PBS) sequence.

In this example, a gene modifying polypeptide contained:
(1) an endonuclease and/or DNA binding domain;
(2) a peptide linker; and
(3) a reverse transcriptase (RT) domain.

Exemplary template RNAs used are given in Table 1A. Nucleotide modifications are noted as follows: phosphorothioate linkages denoted by an asterisk, 2′-O-methyl groups denoted by an ‘m’ preceding a nucleotide. The exemplary gene modifying polypeptide was RNAIVT338, comprising the amino acid sequence of SEQ ID NO: 17522.

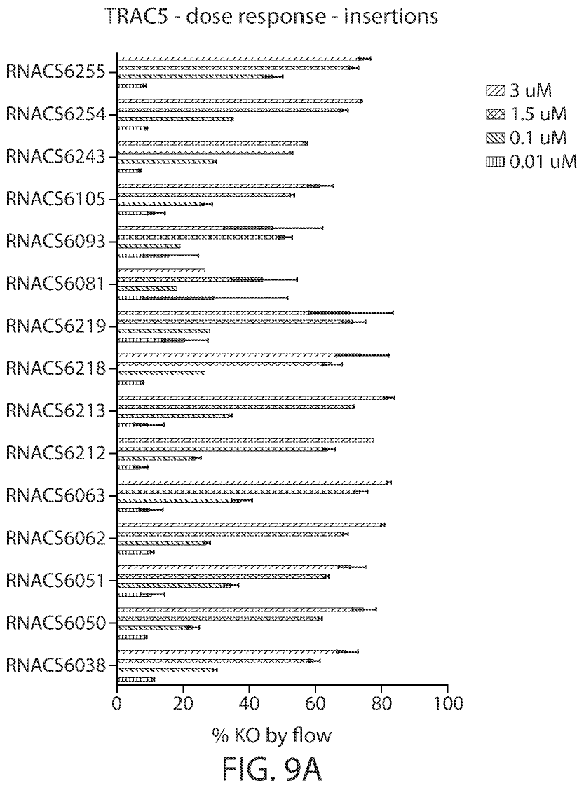

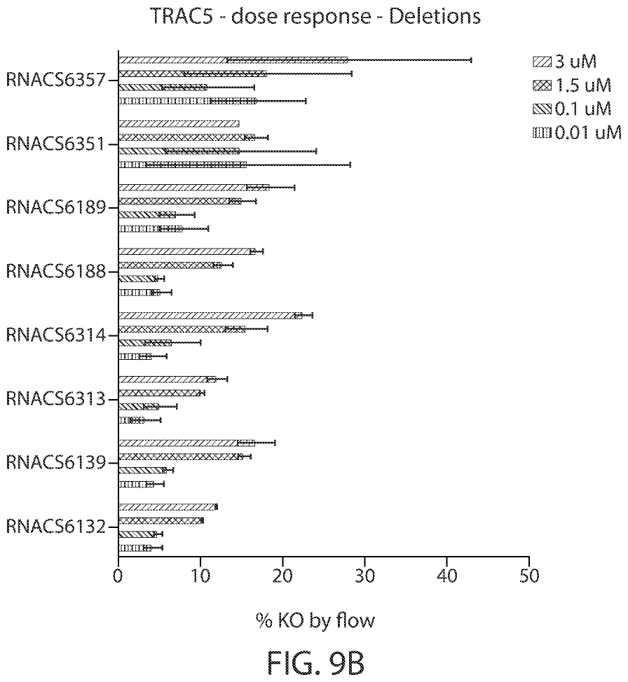

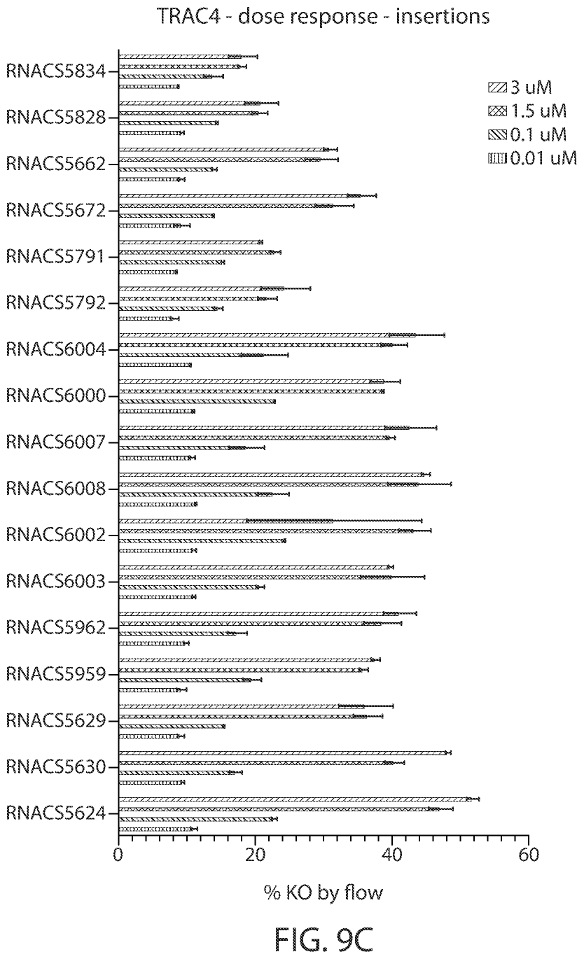

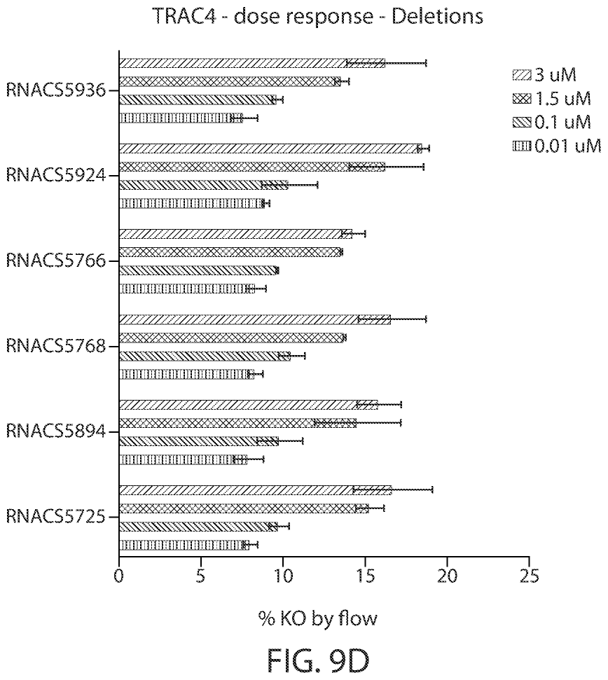

Primary human T cells (CELL161) were activated using TexMACS media containing 5% human AB serum, and cytokines IL-2, IL-15 and IL-7. The heterologous gene modifying system comprising RNAIVT338 gene modifying polypeptide, and a template RNA described above were nucleofected into activated human primary T cells. Specifically, 2 μg of RNAIVT338-encoding mRNA and 0.07 μM tgRNA were added to 25 μL buffer P3 containing 250,000 human T cells. The cells were then nucleofected using the program EO115. After nucleofection, cells were grown at 37° C., 5% CO₂ in TexMACS media supplemented with 5% human AB serum for 3 days prior to running the cells on flow cytometry to assess knockout efficiencies. FIGS. 9A-9D show graphs of % knock out as assessed by flow cytometry for primary human T cells nucleofected with varying doses of select exemplary heterologous gene modifying systems containing TRAC5 insertion template RNAs (FIG. 9A), TRAC5 deletion template RNAs (FIG. 9B), TRAC4 insertion template RNAs (FIG. 9C), or TRAC4 deletion template RNAs (FIG. 9D). The results show that knockout efficiencies of the TRAC4 and TRAC5 template RNAs increased in a dose dependent manner. Generally, the lowest knock out efficiency was observed at 0.01 μM dose and the highest knockout efficiency was observed at 3 μM dose for nearly all template RNAs tested. The TRAC4 template RNA that produced the highest % knockout was RNACS5624 as shown in FIG. 9C which showed a knockout efficiency of 11% at the lowest dose of 0.01 μM and 51.9% at the highest dose of 3 μM. Deletion TRAC4 template RNAs, consistent with above results, achieved editing but were not as efficient as insertion TRAC4 template RNAs. The TRAC4 template RNA that produced the highest % knockout with a deletion was RNACS5924 as shown in FIG. 9D which showed a knockout efficiency of 19%. For TRAC5 template RNAs (FIG. 9A), the template RNA that produced the highest % knockout was RNACS6213 which showed a knockout efficiency of 10% at 0.01 μM dose and 82.3% at 3 μM dose. Deletion TRAC5 template RNAs, consistent with above results, achieved editing but were not as efficient as insertion TRAC5 template RNAs. The TRAC5 template RNA that produced the highest % knockout with a deletion was RNACS6357 as shown in FIG. 9B with a knockout efficiency at the highest dose of 28%.

Taken together, the results further demonstrate successful precise knockout of TRAC with a number of template RNAs designed to produce insertions and deletions of varying sizes, and shows that knock out of TRAC using exemplary heterologous gene modifying systems occurs in a dose dependent manner.

### Example 6: Design and Generation of Template RNAs for B2M Knock Out

This example describes the generation and design of exemplary B2M template RNAs comprising varied spacers, lengths and compositions of heterologous object sequences, and PBS sequences to quantify the activity of template RNAs for knock out of human B2M gene.

To demonstrate the use of gene writing to knockout B2M, two spacers were selected, B2M1 and B2M2, targeting exon1 of the B2M gene. These spacers were chosen because they have demonstrated good indel activity (data not shown). B2M template RNAs were designed to insert 4 or 5 nt sequences. Both insertions were intended to knock out B2M gene due to the introduction of a premature stop codon. Without wishing to be bound by theory, insertions of 4 and 5 nts were selected because DNA heteroduplexes, e.g., created by heterologous gene modifying systems described herein, are more likely to evade recognition by cellular mismatch repair process and result in high editing efficiency. Template RNAs were designed to introduce insertions at the protospacer nick site, because, without wishing to be bound by theory, previous experiments knocking out TRAC (described herein) suggested that insertions at the nick site are more efficient than insertions at the PAM site. In addition to the features described above, the lengths of PBS sequence and the heterologous object sequence were also varied.

To evaluate potential insertion-sequence-specific differences between templates, two additional insertion sequences were also designed. The sequences of 4 nt insertions were TGAT and TAAC, whereas the sequences for 5 nt insertions are TGATG and TAACC. All insertions were designed to introduce at least one stop codon. Without wishing to be bound by theory, mismatch repair is thought to prefer some sequence changes over others.

### Example 7: Evaluating B2M Editing Activity of Exemplary Human Template RNAs Using B2M1 Spacer

This example describes the use of exemplary heterologous gene modifying systems containing a heterologous gene modifying polypeptide and template RNAs comprising B2M1 spacer, varied lengths and compositions of heterologous object sequences, and PBS sequences to quantify the activity of template RNAs for knock out of B2M gene.

In this example, a template RNA contained:
(1) a gRNA spacer;
(2) a gRNA scaffold;
(3) a heterologous object sequence; and
(4) a primer binding site (PBS) sequence.

In this example, a heterologous gene modifying polypeptide contained:
(1) an endonuclease and/or DNA binding domain;
(2) a peptide linker; and
(3) a reverse transcriptase (RT) domain.

Exemplary template RNAs generated are given in Table 1B. Nucleotide modifications are noted as follows: phosphorothioate linkages denoted by an asterisk, 2′-O-methyl groups denoted by an ‘m’ preceding a nucleotide. The exemplary heterologous gene modifying polypeptide is RNAIVT338, comprising the amino acid sequence of SEQ ID NO: 17522.

MPAAKRVKLDGGDKKYSIGLDIGTNSVGWAVITDEYKVPSKKFKVLGNTDRHSIKKNLIGALLF
DSGETAEATRLKRTARRRYTRRKNRICYLQEIFSNEMAKVDDSFFHRLEESFLVEEDKKHERHP
IFGNIVDEVAYHEKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKKNGLFGNLIALS
LGLTPNFKSNFDLAEDAKLQLSKDTYDDDLDNLLAQIGDQYADLFLAAKNLSDAILLSDILRVN
TEITKAPLSASMIKRYDEHHQDLTLLKALVRQQLPEKYKEIFFDQSKNGYAGYIDGGASQEEFY
KFIKPILEKMDGTEELLVKLNREDLLRKQRTFDNGSIPHQIHLGELHAILRRQEDFYPFLKDNR
EKIEKILTFRIPYYVGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDLLFKTNRKVTVKQLK
EDYFKKIECFDSVEISGVEDRFNASLGTYHDLLKIIKDKDFLDNEENEDILEDIVLTLTLFEDR
EMIEERLKTYAHLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQNEKLYLYYLQNGRD
MYVDQELDINRLSDYDVDHIVPQSFLKDDSIDNKVLTRSDKARGKSDNVPSEEVVKKMKNYWRQ
LLNAKLITQRKFDNLTKAERGGLSELDKAGFIKRQLVETRQITKHVAQILDSRMNTKYDENDKL
IREVKVITLKSKLVSDFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPLIETNGETGEIVWDKG
RDFATVRKVLSMPQVNIVKKTEVQTGGFSKESILPKRNSDKLIARKKDWDPKKYGGFDSPTVAY
SVLVVAKVEKGKSKKLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQKQLFVEQHKHYLDEI
IEQISEFSKRVILADANLDKVLSAYNKHRDKPIREQAENIIHLFTLTNLGAPAAFKYFDTTIDR
KRYTSTKEVLDATLIHQSITGLYETRIDLSQLGGDGGAEAAAKEAAAKEAAAKEAAAKALEAEA
AAKEAAAKEAAAKEAAAKAGGTAPLEEEYRLFLEAPIQNVTLLEQWKREIPKVWAEINPPGLAS
TQAPIHVQLLSTALPVRVRQYPITLEAKRSLRETIRKFRAAGILRPVHSPWNTPLLPVRKSGTS
EYRMVQDLREVNKRVETIHPTVPNPYTLLSLLPPDRIWYSVLDLKDAFFCIPLAPESQLIFAFE
WADAEEGESGQLTWTRLPQGFKNSPTLFNEALNRDLQGFRLDHPSVSLLQYVDDLLIAADTQAA
CLSATRDLLMTLAELGYRVSGKKAQLCQEEVTYLGFKIHKGSRSLSNSRTQAILQIPVPKTKRQ
VREFLGKIGYCRLFIPGFAELAQPLYAATRPGNDPLVWGEKEEEAFQSLKLALTQPPALALPSL
DKPFQLFVEETSGAAKGVLTQALGPWKRPVAYLSKRLDPVAAGWPRCLRAIAAAALLTREASKL
TFGQDIEITSSHNLESLLRSPPDKWLTNARITQYQVLLLDPPRVRFKQTAALNPATLLPETDDT
LPIHHCLDTLDSLTSTRPDLTDQPLAQAEATLFTDGSSYIRDGKRYAGAAVVTLDSVIWAEPLP
IGTSAQKAELIALTKALEWSKDKSVNIYTDSRYAFATLHVHGMIYRERGWLTAGGKAIKNAPEI
LALLTAVWLPKRVAVMHCKGHQKDDAPTSTGNRRADEVAREVAIRPLSTQATISAGKRTADGSE
FEKRTADGSEFESPKKKAKVE

Primary human T cells (CELL161) were activated using TexMACS media containing 5% human AB serum, TransAct (1:100) and cytokines IL-2 (20 IU/mL), IL-15 (5 ng/ml) and IL-7 (10 ng/mL). The heterologous gene modifying system comprising mRNA encoding the heterologous gene modifying polypeptide and a template RNA described above were nucleofected into activated human primary T cells. Specifically, 2 μg of RNAIVT338-encoding mRNA and 1.5 μM tgRNA were added to 25 μL buffer P3 containing 250,000 human T cells. The cells were then nucleofected using the program EO115. After nucleofection, cells were grown at 37° C., 5% CO2 in TexMACS media supplemented with 5% human AB serum, IL-15 (5 ng/ml) and IL-7 (10 ng/mL) for 3 days prior to cell lysis and genomic DNA extraction. Editing of the B2M target nucleic acid sequence was assessed using amplicon sequencing (Amp-SEQ) using primers flanking the B2M gene. At the time of cell lysis, one third of the cells from each condition were also cultured for an additional day and ran on the flow cytometer to assess phenotypic knockout efficiency in primary human T cells. To prepare treated cells for flow cytometry, they were collected and stained with Zombie Green Dye (1:500) and Anti-human β2-microglobulin antibody (1:200). The stained cells were analyzed on the iQue3 (Sartorius) flow cytometer. For B2M knockout analysis, cells were gated based on FSC-A and SSC-A. Second, single cells were gated based on FSC-A and FSC-H. Then, live cells, which were negative for Zombie Green Dye, were gated based on FSC-H and BL1-A (blue channel). Finally, B2M negative cells were gated based on FSC-A and YL4-A (yellow channel).

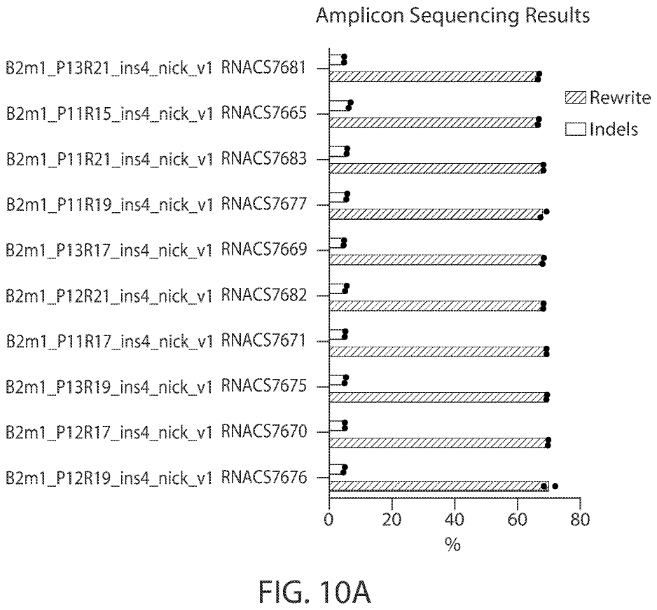

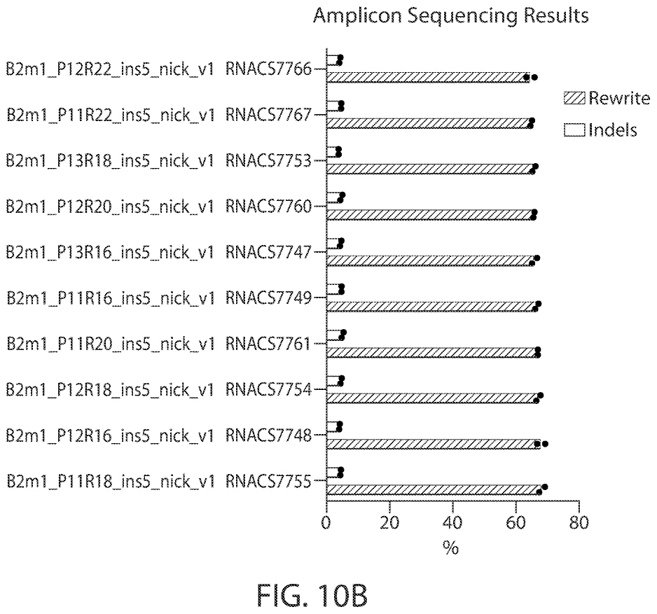

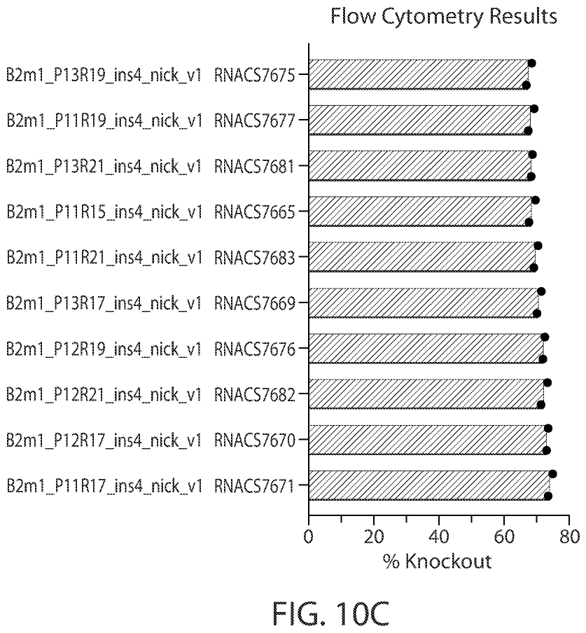

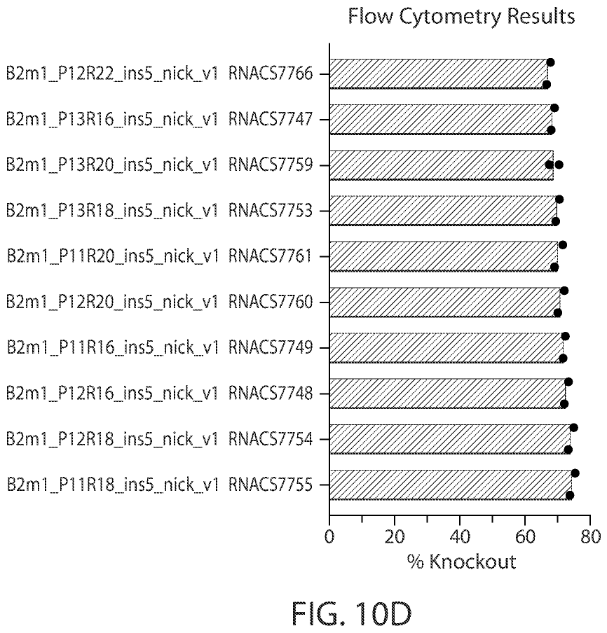

FIG. 10A shows a graph of percent editing as assessed by Amp-SEQ for primary human T cells nucleofected with select exemplary heterologous gene modifying system containing B2M1 template RNAs designed to produce 4 nucleotide TGAT (v1) or TAAC (v2) insertions at the nick site to knock out B2M expression. The results showed that several different B2M1 template RNAs from the cell screen showed >65% rewriting with maximum indels being less than 7%. FIG. 10A shows that the best performing template RNA from the B2M1 screen was RNACS7676 (PBS 12 nt and template 19 nt) producing rewriting levels of up to 70% and indels of 4.7%. Similarly, FIG. 10B shows a graph of percent editing as measured by Amp-SEQ in primary human T cells nucleofected with select exemplary heterologous gene modifying system containing B2M1 template RNAs designed to produce 5 nucleotide TGATG (v1) or TAACC (v2) insertions at the nick site to knock out B2M expression. The results showed that several different B2M1 template RNAs designed to produce 5 nt insertions showed rewriting, and further showed that template RNAs designed to produce 5 nt insertions showed slightly lower activity compared to 4 nt insertion, with the highest 5 nt insertion rewriting being 68%, and indels being 4.1% with RNACS7755 (PBS 11 nt template 18 nt).

FIG. 10C and FIG. 10D show graphs of knockout efficiencies as measured by flow cytometry from the same experiment and the same select template RNAs as shown in FIG. 10A and FIG. 10B, respectively. The flow cytometry data generally corroborate the Amp-SEQ data, confirming that many template RNAs show editing activity sufficient to knock out B2M expression. The top performing template RNAs based on the Amp-SEQ data, RNACS7676 and RNACS7755, showed B2M knockout efficiencies of 71.9% and 74.1% respectively. Without wishing to be bound by theory, discrepancy between rewriting levels as observed by Amp-SEQ vs. flow cytometry may follow from the fact that B2M knockout can result from several editing processes. In addition to rewriting, indels and some other lower frequency imprecise editing events may also result in knockout, leading to Amp-SEQ underestimating the total level of knock out phenotype produced. A further consequence may be that each readout indicates different highest performing template RNAs. For example, the top performing 4 nt insertion template RNA based on flow cytometry (phenotypic knockout) was RNACS7671 whereas Amp-SEQ indicated RNACS7676 as the top performing template RNA.

Taken together, the results demonstrate successful precise knockout of B2M with a number of template RNAs designed to produce insertions of varying sizes and sequences using B2M1 spacer.

### Example 8: Evaluating B2M Editing Activity of Exemplary Human Template RNAs Using B2M2 Spacer

This example describes the use of exemplary heterologous gene modifying systems containing a heterologous gene modifying polypeptide and template RNAs comprising B2M2 spacer, varied lengths and compositions of heterologous object sequences, and PBS sequences to quantify the activity of template RNAs for knock out of B2M gene.

In this example, a template RNA contained:
(1) a gRNA spacer;
(2) a gRNA scaffold;
(3) a heterologous object sequence; and
(4) a primer binding site (PBS) sequence.

In this example, a heterologous gene modifying polypeptide contained:
(1) an endonuclease and/or DNA binding domain;
(2) a peptide linker; and
(3) a reverse transcriptase (RT) domain.

Exemplary template RNAs generated are given in Table 1B. Nucleotide modifications are noted as follows: phosphorothioate linkages denoted by an asterisk, 2′-O-methyl groups denoted by an ‘m’ preceding a nucleotide. The exemplary heterologous gene modifying polypeptide is RNAIVT338, comprising the amino acid sequence of SEQ ID NO: 17522.

Primary human T cells (CELL161) were activated using TexMACS media containing 5% human AB serum, TransAct (1:100) and cytokines IL-2 (20 IU/mL), IL-15 (5 ng/mL) and IL-7 (10 ng/mL). The heterologous gene modifying system comprising mRNA encoding the heterologous gene modifying polypeptide and a template RNA described above were nucleofected into activated human primary T cells. Specifically, 2 μg of RNAIVT338-encoding mRNA and 1.5 μM tgRNA were added to 25 μL buffer P3 containing 250,000 human T cells. The cells were then nucleofected using the program EO115. After nucleofection, cells were grown at 37° C., 5% CO2 in TexMACS media supplemented with 5% human AB serum, IL-15 (5 ng/ml) and IL-7 (10 ng/mL) for 3 days prior to cell lysis and genomic DNA extraction. Editing of the B2M target nucleic acid sequence was assessed using amplicon sequencing (Amp-SEQ) using primers flanking the B2M gene. At the time of cell lysis, one third of the cells from each condition were also cultured for an additional day and ran on the flow cytometer to assess phenotypic knockout efficiency in primary human T cells. To prepare treated cells for flow cytometry, they were collected and stained with Zombie Green Dye (1:500) and Anti-human β2-microglobulin antibody (1:200). The stained cells were analyzed on the iQue3 (Sartorius) flow cytometer. For B2M knockout analysis, cells were gated based on FSC-A and SSC-A. Second, single cells were gated based on FSC-A and FSC-H. Then, live cells, which were negative for Zombie Green Dye, were gated based on FSC-H and BL1-A (blue channel). Finally, B2M negative cells were gated based on FSC-A and YL4-A (yellow channel).

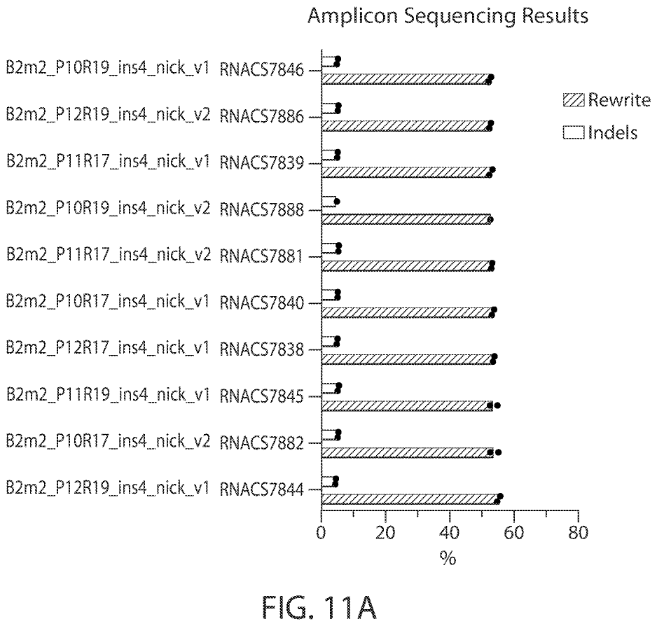

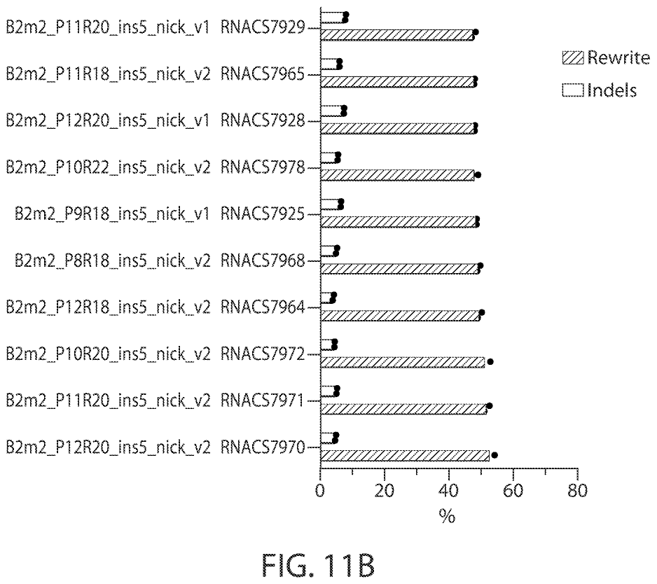

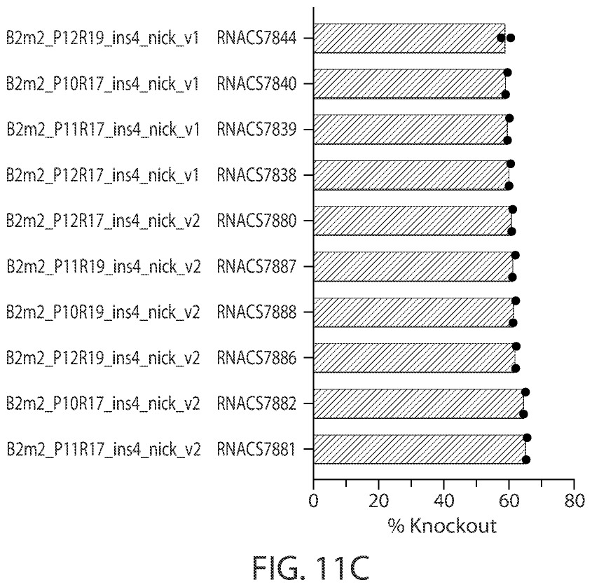

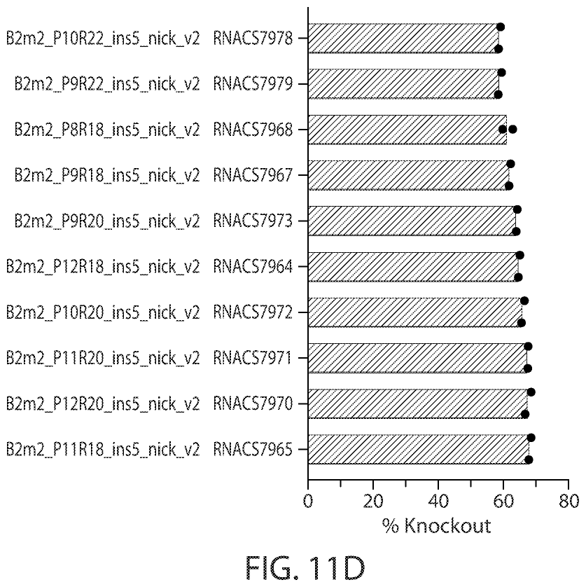

FIG. 11A shows a graph of percent editing as assessed by Amp-SEQ for primary human T cells nucleofected with select exemplary heterologous gene modifying system containing B2M2 template RNAs designed to produce 4 nucleotide TGAT (v1) or TAAC (v2) insertions at the nick site to knock out B2M expression. The results showed that several different B2M2 template RNAs from the cell screen showed >50% rewriting with maximum indels being less than 6%. FIG. 11A shows that the best performing template RNA from the B2M2 screen was RNACS7844 (PBS 12 nt and template 19 nt) producing rewriting levels of up to 55.6% and indels of 4.7%. Similarly, FIG. 11B shows a graph of percent editing as measured by Amp-SEQ in primary human T cells nucleofected with select exemplary heterologous gene modifying system containing B2M2 template RNAs designed to produce 5 nucleotide TGATG (v1) and TAACC (v2) insertions at the nick site to knock out B2M expression. The results showed that several different B2M2 template RNAs designed to produce 5 nt insertions showed rewriting, and further showed that template RNAs designed to produce 5 nt insertions performed a little lower compared to 4 nt insertion, with the highest rewriting being 53% with indels being 4.1 percent with RNACS7970 (PBS 12 nt template 20 nt).

FIG. 11C and FIG. 11D show graphs of knockout efficiencies as measured by flow cytometry from the same experiment and the same select template RNAs as shown in FIG. 11A and FIG. 11B, respectively. The results confirm that a number of B2M2 template RNAs show editing activity sufficient to knock out B2M expression. The results also show that there is considerable difference between editing efficiencies of B2M2 template RNAs as measured by Amp-SEQ vs flow cytometry. For example, the top performing B2M2 template RNA to insert 5 nt insertion (RNACS7970) produced rewriting efficiencies of 52.8% whereas the knockout efficiency as measured by flow cytometry was 67.1%. Similarly, the top performing B2M2 template RNAs to insert 4 nt insertion (RNACS7844) based on the Amp-SEQ data produced rewriting efficiency of 55.6% whereas the knockout efficiency as measured by flow cytometry showed B2M knockout efficiency of 58.7%. Without wishing to be bound by theory, this may be because Amp-SEQ can significantly underestimate editing efficiency. Two major reasons may explain the under-estimation. First, B2M knockout can result from several editing processes (e.g., rewriting and indels). Second, the sequencing quality (e.g., sequencing errors or PCR errors) may also further underestimate the knockout efficiency by Amp-SEQ. The highest knockout efficiencies achieved as measured by flow cytometry were 65% for 4 nt insertion (RNACS7881) and 67.7% for 5 nt insertion (RNACS7965).

Taken together, the results demonstrate successful precise knockout of B2M with a number of template RNAs designed to produce insertions of varying sizes, and sequences, using B2M2 spacers.

### Example 9: Evaluating B2M Editing Activity of Exemplary Human Template RNAs with Different Insert Sequences Using B2M1 and B2M2 Spacers

This example describes the use of exemplary heterologous gene modifying systems containing a heterologous gene modifying polypeptide and template RNAs comprising B2M1 and B2M2 spacers, varied lengths and compositions of heterologous object sequences, and PBS sequences to quantify the activity of template RNAs for knock out of B2M gene.

In this example, a template RNA contained:
(1) a gRNA spacer;
(2) a gRNA scaffold;
(3) a heterologous object sequence; and
(4) a primer binding site (PBS) sequence.

In this example, a heterologous gene modifying polypeptide contained:
(1) an endonuclease and/or DNA binding domain;
(2) a peptide linker; and
(3) a reverse transcriptase (RT) domain.

Exemplary template RNAs generated are given in Table 1B. Nucleotide modifications are noted as follows: phosphorothioate linkages denoted by an asterisk, 2′-O-methyl groups denoted by an ‘m’ preceding a nucleotide. The exemplary heterologous gene modifying polypeptide is RNAIVT338, comprising the amino acid sequence of SEQ ID NO: 17522.

Primary human T cells (CELL161) were activated using TexMACS media containing 5% human AB serum, TransAct (1:100) and cytokines IL-2 (20 IU/mL), IL-15 (5 ng/ml) and IL-7 (10 ng/mL). The heterologous gene modifying system comprising mRNA encoding the heterologous gene modifying polypeptide and a template RNA described above were nucleofected into activated human primary T cells. Specifically, 2 μg of RNAIVT338-encoding mRNA and 1.5 μM tgRNA were added to 25 μL buffer P3 containing 250,000 human T cells. The cells were then nucleofected using the program EO115. After nucleofection, cells were grown at 37° C., 5% CO₂ in TexMACS media supplemented with 5% human AB serum, IL-15 (5 ng/ml) and IL-7 (10 ng/mL) for 4 days. To prepare treated cells for flow cytometry, they were collected and stained with Zombie Green Dye (1:500) and Anti-human B2-microglobulin antibody (1:200). The stained cells were analyzed on the iQue3 (Sartorius) flow cytometer. For B2M knockout analysis, cells were gated based on FSC-A and SSC-A. Second, single cells were gated based on FSC-A and FSC-H. Then, live cells, which were negative for Zombie Green Dye, were gated based on FSC-H and BL1-A (blue channel). Finally, B2M negative cells were gated based on FSC-A and YL4-A (yellow channel).

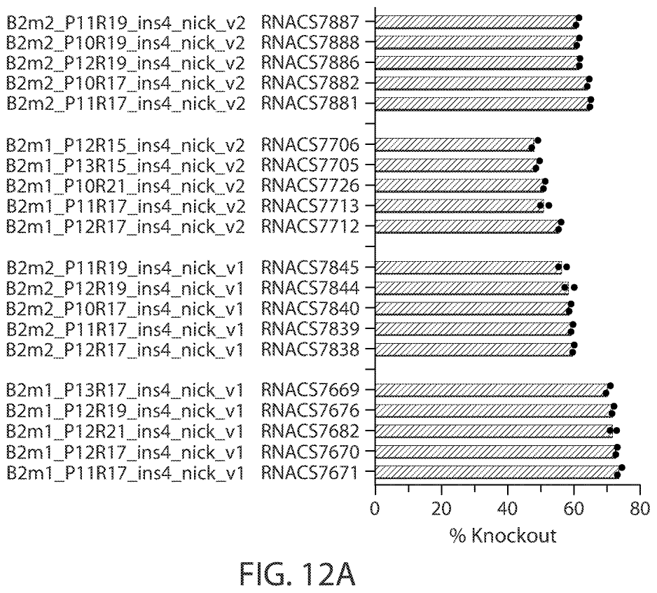

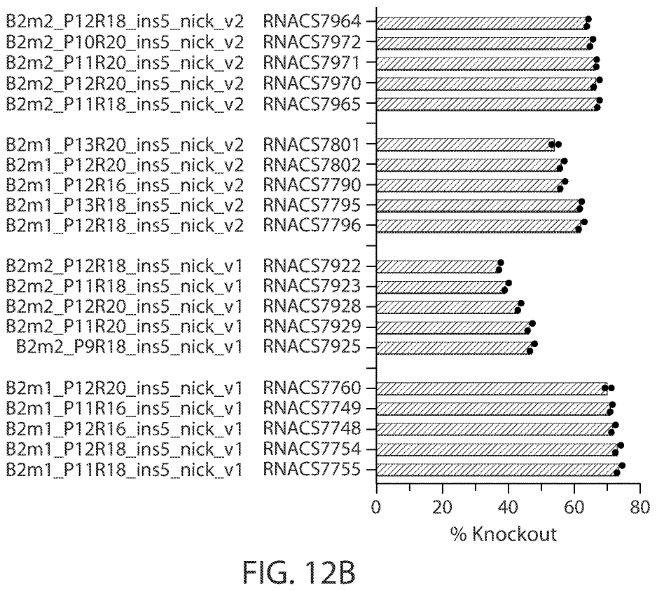

FIGS. 12A-12B show graphs of percent knockout as assessed by flow cytometry for primary human T cells nucleofected with select exemplary heterologous gene modifying system containing B2M1 or B2M2 template RNAs designed to produce 4 nucleotide insertions (TGAT (v1) or TAAC (v2)) insertions or 5 nt insertions (TGATG (v1) or TAACC (v2)) at the nick site to knock out B2M expression. The results show that template RNAs containing different combinations of insertion compositions and spacers were able to facilitate rewriting in a manner sufficient for B2M knock out. Differences in rewriting efficiency were observed with different insert compositions in a spacer dependent manner. For example, the best template RNA to insert TGAT for B2M1 was RNACS7671 (73.9% B2M knockout) whereas the highest editing efficiency achieved for inserting TAAC for B2M1 was 55.8% knockout (RNACS7712). For B2M2 spacer, the top performing template RNA to insert TGAT was RNACS7838 (60% B2M knockout) whereas the best template RNA to insert TGAC was RNACS7881 (65.05% B2M knockout). Similarly, for 5 nt insertions, the best template RNA to insert TGATG for B2M1 was RNACS7755 (74.1% B2M knockout) whereas the highest editing efficiency achieved for inserting TAACC for B2M1 was 62.5% knockout (RNACS7796). For B2M2 spacer, the top performing template RNA to insert TGATG was RNACS7925 (47.5% B2M knockout) whereas the best template RNA to insert TAACC was RNACS7965 (67.5% B2M knockout). The results suggest that B2M1 template RNAs show higher rewriting with insertion compositions of TGAT or TGATG, whereas B2M2 template RNAs show higher rewriting with insertion compositions of TAAC or TAACC. More generally, the results suggest that particular combinations of spacers and heterologous object sequence compositions may provide higher rewriting efficiency over other combinations of said elements.

Taken together, the results demonstrate successful precise knockout of B2M in a spacer composition and insertion composition dependent manner.

Based upon the differences in rewriting observed for different compositions of 4 and 5 nt insertion sequences, an exhaustive screen of the 4 or 5 nt insertion compositions is performed to identify the top performing composition. Template RNAs are designed to insert 4 or 5 nt insertions where every possible composition is tested (see, e.g., Tables 3B and 4B). Template RNAs are designed to insert NNNN or NNNNN where N can be A, T/U, G or C. 1024 different compositions of 5 nt insertions and 256 different compositions of 4 nt insertions are tested. Table 4B contains all the insertion compositions that are tested. It is expected that various insertions sequences will be identified that perform better than other sequence based on rewriting rates achieved.

### Example 10: Evaluating the Dose Response of Exemplary Human Template RNAs with Different Insert Sequences in B2M

This example describes the use of exemplary heterologous gene modifying systems containing a heterologous gene modifying polypeptide and varying doses of template RNAs comprising B2M1 and B2M2 spacers, varied lengths and compositions of heterologous object sequences, and PBS sequences to quantify the dose response of template RNAs for knocking out the B2M gene.

In this example, a template RNA contained:
(1) a gRNA spacer;
(2) a gRNA scaffold;
(3) a heterologous object sequence; and
(4) a primer binding site (PBS) sequence.

In this example, a heterologous gene modifying polypeptide contained:
(1) an endonuclease and/or DNA binding domain;
(2) a peptide linker; and
(3) a reverse transcriptase (RT) domain.

Exemplary template RNAs generated are given in Table 1B. Nucleotide modifications are noted as follows: phosphorothioate linkages denoted by an asterisk, 2′-O-methyl groups denoted by an ‘m’ preceding a nucleotide. The exemplary heterologous gene modifying polypeptide is RNAIVT338, comprising the amino acid sequence of SEQ ID NO: 17522.

Primary human T cells (CELL161) were activated using TexMACS media containing 5% human AB serum, TransAct (1:100) and cytokines IL-2 (20 IU/mL), IL-15 (5 ng/ml) and IL-7 (10 ng/mL). The heterologous gene modifying system comprising mRNA encoding the heterologous gene modifying polypeptide, and a template RNA described above were nucleofected into activated human primary T cells. Specifically, 2 μg of RNAIVT338-encoding mRNA and 0.01, 0.1, 1.5, or 3 μM of template RNA were added to 25 μL buffer P3 containing 250,000 human T cells. The cells were then nucleofected using the program EO115. After nucleofection, cells were grown at 37° C., 5% CO₂ in TexMACS media supplemented with 5% human AB serum, IL-15 (5 ng/mL) and IL-7 (10 ng/mL) for 4 days. To prepare treated cells for flow cytometry, they were collected and stained with Zombie Green Dye (1:500) and Anti-human B2-microglobulin antibody (1:200). The stained cells were analyzed on the iQue3 (Sartorius) flow cytometer. For B2M knockout analysis, cells were gated based on FSC-A and SSC-A. Second, single cells were gated based on FSC-A and FSC-H. Then, live cells, which were negative for Zombie Green Dye, were gated based on FSC-H and BL1-A (blue channel). Finally, B2M negative cells were gated based on FSC-A and YL4-A (yellow channel).

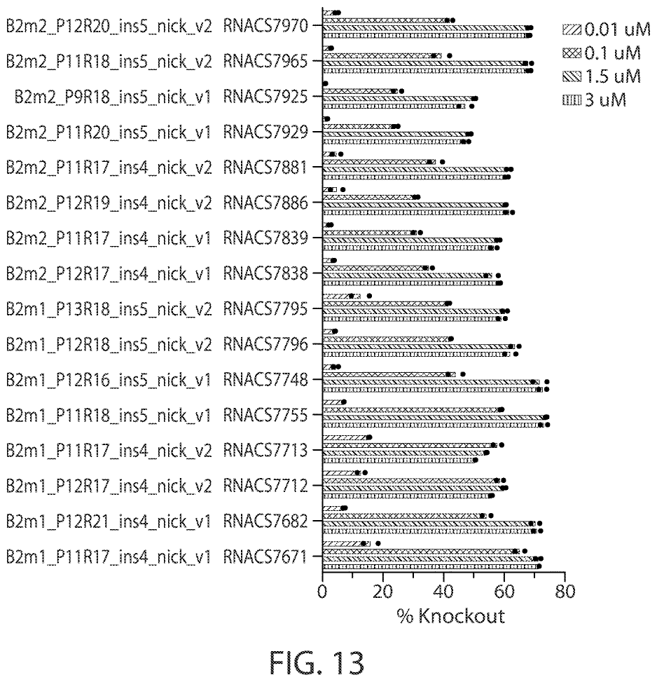

FIG. 13 shows graphs of percent knockout as assessed by flow cytometry for primary human T cells nucleofected with select exemplary heterologous gene modifying system containing B2M1 or B2M2 template RNAs designed to produce 4 nucleotide insertions (TGAT (v1) or TAAC (v2)) insertions or 5 nt insertions (TGATG (v1) or TAACC (v2)) at the nick site to knock out B2M expression. The results further show that a number of B2M1 and B2M2 template RNAs facilitate rewriting in a manner sufficient to knock out B2M. Knockout efficiencies of B2M1 and B2M2 template RNAs were found to increase in a dose dependent manner, with lowest overall efficiency observed at 0.01 μM dose and highest knockout efficiency observed at 1.5 μM or 3 μM dose. For B2M1, the best performing template RNA at 0.01 μM was RNACS7671 (TGAT insertion) as shown in FIG. 13 with knockout efficiency of 15.8% at the lowest dose of 0.01 μM and 71.6% at an apparently saturating dose of 3 μM. For B2M2, the highest knockout achieved at 0.01 μM was 4.6% with RNACS7970 (TAACC). The knockout efficiency increased to 68.35% at an apparently saturating dose of 1.5 μM. Overall, the highest knockout efficiency achieved was 74% with RNACS7755 (B2M1 spacer and TGATG insertion) at 1.5 μM dose.

The results show that exemplary heterologous gene modifying systems comprising template RNAs described herein can effectively knockout B2M gene expression with knockout efficiencies increasing in a template RNA dose dependent manner.

### Example 11: Evaluating Combined Gene Editing Activity of Exemplary TRAC Human Template RNAs with Gene Modifying Systems

This example describes the use of a first exemplary heterologous gene modifying system containing a heterologous gene modifying polypeptide and 2 template RNAs to produce an insertion of 5 nucleotides (TAGTG) in the first exon of the human TRAC gene and an insertion of 5 nucleotides (TAGTG) in the first exon of the Beta 2 Microglobulin (B2M) gene in primary human T cells, and a second exemplary retrotransposon gene modifying system containing a retrotransposon gene modifying polypeptide and a template RNA to insert expression cassettes for a chimeric antigen receptor (CAR) or GFP into the genome of the primary human T cells, as well as quantification of said gene modifying activities. The insertions into TRAC and B2M generated a frameshift that resulted in a non-functional gene (also referred to as a knock-out).

In this example, the template RNA of the first heterologous gene modifying system contained:
(1) a gRNA spacer;
(2) a gRNA scaffold;
(3) a heterologous object sequence; and
(4) a primer binding site (PBS) sequence.

The exemplary template RNAs generated and used in the first heterologous gene modifying system are given in Table E7. Nucleotide modifications are noted as follows: phosphorothioate linkages denoted by an asterisk, 2′-O-methyl groups denoted by an ‘m’ preceding a nucleotide.

TABLE E7
Exemplary Template RNAs and Sequences

| | | | SEQ |
|---|---|---|---|
| Name | Descriptor | Sequence | ID NO |
| RNACS5630 | TRAC4_ | mG*mA*mG*rArArU | 15,004 |
| | Ins 5 | rCrArArArArUrCr | |
| | Nick | GrGrUrGrArArUrG | |
| | P9R18 | rUrUrUrUrArGrAm | |
| | | GmCmUmAmGmAmAmA | |
| | | mUmAmGmCrArArGr | |
| | | UrUrArArArArUrA | |
| | | rArGrGrCrUrArGr | |
| | | UrCrCrGrUrUrArU | |
| | | rCrAmAmCmUmUmGm | |
| | | AmAmAmAmAmGmUmG | |
| | | mGmCmAmCmCmGmAm | |
| | | GmUmCmGmGmUmGmC | |
| | | rCrUrGrUrCrUrGr | |
| | | CrCrUrArUrUrUrA | |
| | | rGrUrGrCrArCrCr | |
| | | GrA*mU*mU*mU | |
| RNACS7755 | B2m1_ | mA*mC*mU*rCrArC | 15,005 |
| | P11R18_ | rGrCrUrGrGrArUr | |
| | ins5_ | ArGrCrCrUrCrCrG | |
| | nick_ | rUrUrUrUrArGrAm | |
| | v1 | GmCmUmAmGmAmAmA | |
| | | mUmAmGmCrArArGr | |
| | | UrUrArArArArUrA | |
| | | rArGrGrCrUrArGr | |
| | | UrCrCrGrUrUrArU | |
| | | rCrAmAmCmUmUmGm | |
| | | AmAmAmAmAmGmUmG | |
| | | mGmCmAmCmCmGmAm | |
| | | GmUmCmGmGmUmGmC | |
| | | rUrUrUrCrUrGrGr | |
| | | CrCrUrGrGrArUrG | |
| | | rArUrGrGrGrCrUr | |
| | | ArUrCrC*mA*mG*m | |
| | | C | |

Table E7A shows the sequences of Table E7 without chemical modifications. In some embodiments, the sequences of Table E7A may be used without chemical modifications, or with one or more chemical modifications.

TABLE E7A
Table E7 Sequences without Chemical Modifications

| | | | SEQ |
|---|---|---|---|
| RNACS | Name | Sequence | ID NO |
| RNACS | TRAC4_ | GAGAAUCAAAAUCGGUGAAUGUUUU | 15,006 |
| 5630 | Ins5_ | AGAGCUAGAAAUAGCAAGUUAAAAU | |
| | Nick | AAGGCUAGUCCGUUAUCAACUUGAA | |
| | P9R18 | AAAGUGGCACCGAGUCGGUGCCUGU | |
| | | CUGCCUAUUUAGUGCACCGAUUU | |
| RNACS | B2m1_ | ACUCACGCUGGAUAGCCUCCGUUUU | 15,007 |
| 7755 | P11R18_ | AGAGCUAGAAAUAGCAAGUUAAAAU | |
| | ins5_ | AAGGCUAGUCCGUUAUCAACUUGAA | |
| | nick_ | AAAGUGGCACCGAGUCGGUGCUUUC | |
| | v1 | UGGCCUGGAUGAUGGGCUAUCCAGC | |

In this example, exemplary heterologous gene modifying polypeptides of the first heterologous gene modifying system contained:
(1) an endonuclease and/or DNA binding domain;
(2) a peptide linker; and
(3) a reverse transcriptase (RT) domain,
   where (1), (2), and (3) are heterologous to each other. Accordingly, the first heterologous gene modifying polypeptides in this Example were examples of heterologous gene modifying polypeptides.

The exemplary gene heterologous modifying polypeptide of the first gene modifying system was RNAIVT1564, and comprised the amino acid sequence indicated in Table E8.

TABLE E8
Exemplary heterologous gene modifying
polypeptide and Sequence

| | | SEQ ID |
|---|---|---|
| Name | AA sequence | NO |
| RNAIVT1564 | MPAAKRVKLDGGDKKYSIGLDIGTNSVGWA | 15,008 |
| | VITDEYKVPSKKFKVLGNTDRHSIKKNLIG | |
| | ALLFDSGETAEATRLKRTARRRYTRRKNRI | |
| | CYLQEIFSNEMAKVDDSFFHRLEESFLVEE | |
| | DKKHERHPIFGNIVDEVAYHEKYPTIYHLR | |
| | KKLVDSTDKADLRLIYLALAHMIKFRGHFL | |
| | IEGDLNPDNSDVDKLFIQLVQTYNQLFEEN | |
| | PINASGVDAKAILSARLSKSRRLENLIAQL | |
| | PGEKKNGLFGNLIALSLGLTPNFKSNFDLA | |
| | EDAKLQLSKDTYDDDLDNLLAQIGDQYADL | |
| | FLAAKNLSDAILLSDILRVNTEITKAPLSA | |
| | SMIKRYDEHHQDLTLLKALVRQQLPEKYKE | |
| | IFFDQSKNGYAGYIDGGASQEEFYKFIKPI | |
| | LEKMDGTEELLVKLNREDLLRKQRTFDNGS | |
| | IPHQIHLGELHAILRRQEDFYPFLKDNREK | |
| | IEKILTFRIPYYVGPLARGNSRFAWMTRKS | |
| | EETITPWNFEEVVDKGASAQSFIERMTNFD | |
| | KNLPNEKVLPKHSLLYEYFTVYNELTKVKY | |
| | VTEGMRKPAFLSGEQKKAIVDLLFKTNRKV | |
| | TVKQLKEDYFKKIECFDSVEISGVEDRFNA | |
| | SLGTYHDLLKIIKDKDFLDNEENEDILEDI | |
| | VLTLTLFEDREMIEERLKTYAHLFDDKVMK | |
| | QLKRRRYTGWGRLSRKLINGIRDKQSGKTI | |
| | LDFLKSDGFANRNFMQLIHDDSLTFKEDIQ | |
| | KAQVSGQGDSLHEHIANLAGSPAIKKGILQ | |
| | TVKVVDELVKVMGRHKPENIVIEMARENQT | |
| | TQKGQKNSRERMKRIEEGIKELGSQILKEH | |
| | PVENTQLQNEKLYLYYLQNGRDMYVDQELD | |
| | INRLSDYDVDHIVPQSFLKDDSIDNKVLTR | |
| | SDKARGKSDNVPSEEVVKKMKNYWRQLLNA | |
| | KLITQRKFDNLTKAERGGLSELDKAGFIKR | |
| | QLVETRQITKHVAQILDSRMNTKYDENDKL | |
| | IREVKVITLKSKLVSDFRKDFQFYKVREIN | |
| | NYHHAHDAYLNAVVGTALIKKYPKLESEFV | |
| | YGDYKVYDVRKMIAKSEQEIGKATAKYFFY | |
| | SNIMNFFKTEITLANGEIRKRPLIETNGET | |
| | GEIVWDKGRDFATVRKVLSMPQVNIVKKTE | |
| | VQTGGFSKESILPKRNSDKLIARKKDWDPK | |
| | KYGGFDSPTVAYSVLVVAKVEKGKSKKLKS | |
| | VKELLGITIMERSSFEKNPIDFLEAKGYKE | |
| | VKKDLIIKLPKYSLFELENGRKRMLASAGE | |
| | LQKGNELALPSKYVNFLYLASHYEKLKGSP | |
| | EDNEQKQLFVEQHKHYLDEIIEQISEFSKR | |
| | VILADANLDKVLSAYNKHRDKPIREQAENI | |
| | IHLFTLTNLGAPAAFKYFDTTIDRKRYTST | |
| | KEVLDATLIHQSITGLYETRIDLSQLGGDS | |
| | GGSSGGSSGSETPGTSESATPESSGGSSGG | |
| | SSTLNIEDEYRLHETSKEPDVSLGSTWLSD | |
| | FPQAWAETGGMGLAVRQAPLIIPLKATSTP | |
| | VSIKQYPMSQEARLGIKPHIQRLLDQGILV | |
| | PCQSPWNTPLLPVKKPGTNDYRPVQDLREV | |
| | NKRVEDIHPTVPNPYNLLSGLPPSHQWYTV | |
| | LDLKDAFFCLRLHPTSQPLFAFEWRDPEMG | |
| | ISGQLTWTRLPQGFKNSPTLFNEALHRDLA | |
| | DFRIQHPDLILLQYVDDLLLAATSELDCQQ | |
| | GTRALLQTLGNLGYRASAKKAQICQKQVKY | |
| | LGYLLKEGQRWLTEARKETVMGQPTPKTPR | |
| | QLREFLGKAGFCRLFIPGFAEMAAPLYPLT | |
| | KPGTLFNWGPDQQKAYQEIKQALLTAPALG | |
| | LPDLTKPFELFVDEKQGYAKGVLTQKLGPW | |
| | RRPVAYLSKKLDPVAAGWPPCLRMVAAIAV | |
| | LTKDAGKLTMGQPLVILAPHAVEALVKQPP | |
| | DRWLSNARMTHYQALLLDTDRVQFGPVVAL | |
| | NPATLLPLPEEGLQHNCLDILAEAHGTRPD | |
| | LTDQPLPDADHTWYTDGSSLLQEGQRKAGA | |
| | AVTTETEVIWAKALPAGTSAQRAELIALTQ | |
| | ALKMAEGKKLNVYTDSRYAFATAHIHGEIY | |
| | RRRGWLTSEGKEIKNKDEILALLKALFLPK | |
| | RLSIIHCPGHQKGHSAEARGNRMADQAARK | |
| | AAITETPDTSTLLIENSSPSGGSKRTADGS | |
| | EFEKRTADGSEFESPKKKAKVE | |

In this example, exemplary gene modifying polypeptides of the second retrotransposon gene modifying system contained:
(1) an endonuclease and/or DNA binding domain; and
(2) a reverse transcriptase (RT) domain,
   where (1) and (2) are both derived from a retrotransposon (and in this Example, from the same retrotransposons). Accordingly, the second gene modifying polypeptides in this Example were examples of retrotransposon gene modifying polypeptides.

The second retrotransposon gene modifying system used in this Example comprised either of two exemplary retrotransposon gene modifying polypeptides: RNAIVT696 (SEQ ID NO:15,002) and RNAIVT1123 (SEQ ID NO: 15,003), and amino acid sequences are given in Table E9.

TABLE E9
Exemplary gene modifying polypeptide and Sequences

| | | SEQ ID |
|---|---|---|
| Name | AA sequence | NO |
| RNAIVT696 | MDEYQRSLSRPLLTIMSINIEGLSLAKEELLAKMSE | 15,002 |
| | DISCDILCIQETHRDITMRRPKILGMQLAVERPHRQ | |
| | YGSAIFVRSGVAISATSLTEVNNIEILSVELDSCTV | |
| | SSLYKPPGADFYFTPPTSCHNHEAHFVVGDENSHSC | |
| | VWGYDEDDRNGEAVLTWADNSRMSLLHDSKLPPSFN | |
| | SGRWKRGYNPDLIFVKESISHQCTKRVLNPIPNTQH | |
| | RPICCVAYAAVRPKSVPFRRRYNFNKANWTKFTETL | |
| | EAAISDIEPSIENYDLFVEAVKRSSRLSIPRGCRTS | |
| | YLPGLNEESLNQLQEYLRLFQENPYSDGTIAAGQKL | |
| | STALANAKKDRWIELLENLDMSKSSRKAWQLLRRLD | |
| | SDPLVNPGHANVTPDQIAHQLIQNGKTNCSRIKMKI | |
| | NRVPELETHQLSSPLNLKELREAIKRCKTGKAPGLD | |
| | DLMMEQIKHLGAKAENWLLKFYNQCLAHKQIPRAWR | |
| | KTKIIAILKPGKDASNARNYRPISLLCHLYKVYERM | |
| | LLNRLGPVIEPKLIAQQAGFRPGKNCTGQILHLTEH | |
| | IEEGYEKGCITGTVFVDLTAAYDTVQHRKMLHKVYH | |
| | ITRDFDFTKTVQTLLENRSFYVEFQGQKSRWRRQKN | |
| | GLPQGSVLAPTLFNIFTNDQPQPPLTKSFIYADDLG | |
| | LTTQAKDFETVEKQLTNALKDLSSYYKENHLKPNPA | |
| | KTQVCAFHLRNREANRKLKVTWEGQELEHCFHPKYL | |
| | GVTLDRTLTYRKHCMNTKHKVAARNNILRKLTGSAW | |
| | GADPQVIRTSALALSFSTAEYACPVWHKSAHAKQVD | |
| | IALNETCRIITGCLKPTPVDKLYKLAGIAPPDVRRE | |
| | VAANGERKKVEHCESHPLHGYHPPPTRLKSRKGFMR | |
| | TTTPLDVPPAAARVSLWAAKPGNSNWMAPQEGLPPG | |
| | ANQEWATWKSLNRLRSGVGRSKDNLARWHYLEESST | |
| | LCDCGAEQTTQHMYACPQCPASCTEEELFKATDNAV | |
| | AVARFWSKTIGGGSPKKKRKVSGSETPGTSESATPE | |
| | SVSGWRLFKKIS | |
| RNAIVT1123 | MDSTAHPNQGRGLEKVSQTLPALQTPGQHTAAGGSS | 15,003 |
| | PLSGRNQRKNTKKLLLGAWNIRTLLDRENTPRPERR | |
| | TALIGKELARYNIDIAALSETRLPEEGSLSEPTTGY | |
| | TFFWKGRASNEDRIHGVGLAIKTSLLKQLPDLPVGI | |
| | SERLMKIRLPLSKDRYATIISAYAPTLTSTEETIEQ | |
| | FYSDLSAVLHSVPTNDKLILLGDFNARVGQDHERWK | |
| | GVLGKHGVGKMNNNGLLLLSKCSEFELTITNTVFRM | |
| | ANKYKTTWMHPRSKQWHLIDYIIVRRRDIQDVKITR | |
| | AMRGAECWTDHRLVRATLQMRIAPRHPKRAQTVRAF | |
| | YNVSRLRDPSYLQTFQSCLDDKLSAKGPLTGSSTEK | |
| | WNQFRDAVKETSKAVLGPKQRNHQDWFDENNTAIED | |
| | LLSKKNKAFMEWQNNPNSAPKKDRFKSLQATAQREI | |
| | RKMQDRWWEKKAEEIQRFADMKNYKQFFSALKTVYG | |
| | PLKPTTTPLLSSDGDTLIKDKKGISNRWKEHFSQLL | |
| | NRPSSVDQSALDQIPQNRTIEQLDVPPSIEEVQKAI | |
| | KQMSAGKAPGKDGIPTEVYKALNGKALQAFHIVLTS | |
| | IWEEEDMPPELRDASIVALYKNKGSRAACDNYRGIS | |
| | LLSTAGKILARVILNRLLSSVSEQNLPESQCGFRPD | |
| | RSTIDMVFTVRQMQEKCLEQNLSLYIVFIDLTKAFD | |
| | TVNRDALWVILSKLGCPAKFVKLIQLFHVDMTGEVL | |
| | SGGETSDRFNISNGVKQGCVLAPVLFNLFFTQVLRH | |
| | AVMDLDLGVYIKYRLDGSLFDLRRLTAKTKTTERLI | |
| | LEALFADDCALMAHQENHLQTIVDRFSTATKLFGLT | |
| | ISLSKTEVLFQPAPGRPTNQPCITIDGTQLSNVNTF | |
| | KYLGSTIANDGSLDHEINARIQKASQALGRLRCKVL | |
| | QHRGVSTATKLKVYNAVVLSSLLYGCETWTLYRKHM | |
| | KQLEQFHQRSLRSIMRIRWQDRITNQEVLDRANSTS | |
| | IEVMVLKTQLRWSGHVIRMDPQRIPRQVFYGELSAG | |
| | LRKQGRPKKRFKDQLKSNLKWAGITPKQLELAASDR | |
| | SSWRTHINHAATTFEDERRRRLAAARERRHQATTAP | |
| | PVTTGVPCPMCHKLCASAFGLQSHMRVHRRSGSETP | |
| | GTSESATPESVSGWRLFKKIS | |

Exemplary templates RNAs comprising sequences capable of binding the exemplary retrotransposon gene modifying polypeptides included RNAIVT381 for RNAIVT696 gene modifying polypeptide and RNAIVT610 and RNAIVT1200 for RNAIVT1123 gene modifying polypeptide. Sequences are given in Table E10.

TABLE E10
Exemplary retrotransposon gene modifying RNA template and Sequences

| | | SEQ ID |
|---|---|---|
| Name | RNA sequence (5′ to 3′) | NO |
| RNAIVT381 | gggggacacggaaagagccuccccgaagauugagugaau | 15,009 |
| | ucagucgggcguccccugggcaacguuucuuguaagcgg | |
| | ccgaucuuuccaccccaaaagcauuggaugaGUUUACGG | |
| | AUCCGAAUUCUCGACGGAUCGAUCCGAACAAACGACCCA | |
| | ACACCCGUGCGUUUUAUUCUGUCUUUUUAUUGCCGAUCC | |
| | CCCGGCCGCUUUACUUGUACAGCUCGUCCAUGCCGAGAG | |
| | UGAUCCCGGCGGCGGUCACGAACUCCAGCAGGACCAUGU | |
| | GAUCGCGCUUCUCGUUGGGGUCUUUGCUCAGGGCGGACU | |
| | GGGUGCUCAGGUAGUGGUUGUCGGGCAGCAGCACGGGGC | |
| | CGUCGCCGAUGGGGGUGUUCUGCUGGUAGUGGUCGGCGA | |
| | GCUGCACGCUGCCGUCCUCGAUGUUGUGGCGGAUCUUGA | |
| | AGUUCACCUUGAUGCCGUUCUUCUGCUUGUCGGCCAUGA | |
| | UAUAGACGUUGUGGCUGUUGUAGUUGUACUCCAGCUUGU | |
| | GCCCCAGGAUGUUGCCGUCCUCCUUGAAGUCGAUGCCCU | |
| | UCAGCUCGAUGCGGUUCACCAGGGUGUCGCCCUCGAACU | |
| | UCACCUCGGCGCGGGUCUUGUAGUUGCCGUCGUCCUUGA | |
| | AGAAGAUGGUGCGCUCCUGGACGUAGCCUUCGGGCAUGG | |
| | CGGACUUGAAGAAGUCGUGCUGCUUCAUGUGGUCGGGGU | |
| | AGCGGCUGAAGCACUGCACGCCGUAGGUCAGGGUGGUCA | |
| | CGAGGGUGGGCCAGGGCACGGGCAGCUUGCCGGUGGUGC | |
| | AGAUGAACUUCAGGGUCAGCUUGCCGUAGGUGGCAUCGC | |
| | CCUCGCCCUCGCCGGACACGCUGAACUUGUGGCCGUUUA | |
| | CGUCGCCGUCCAGCUCGACCAGGAUGGGCACCACCCCGG | |
| | UGAACAGCUCCUCGCCCUUGCUCACCAUGGUGGCUUUAC | |
| | CAACAGUACCGGAAUGCCAAGCUUGGGUCcuguguucug | |
| | gggcaaacccguugcgaaaaagaacguucacggcgacua | |
| | cugcacuuauauacgguucucccccacccucgggaaaaa | |
| | ggcggagccaguacacgacaucacuuucccaguuuaccc | |
| | cgcgccaccuucucuaggcaccggaucaauugccgaccc | |
| | cuccccccaacuucucggggacugugggcuauuugaaau | |
| | guauuuguuguagcaaugcuuuugacacgaaauaaauaa | |
| | a | |
| RNAIVT610 | ggguguauggggugcucagggagggguaguaucucuggu | 15,010 |
| | auggagggcuugucgugcccuccuagggcagcucuccag | |
| | ccucugacccccaccugacacccagcucucacuuguggc | |
| | ucccaguagcugcuagcauguggcagcggccacaccccg | |
| | ggcaacggcuucgacaggccggcuaaaccuugugagggu | |
| | agccaucgggucaucgaccccuggugaaccagggcuuug | |
| | cucacccagcaugugaagacugcuucggcugaacagacg | |
| | gaagaaaccaauaagaagguucaacggcugagagggcga | |
| | cgcagcaaagcacuguggagugcuuagggcguguuggag | |
| | cacugccacuggacccaggcuuccaacgccgagagagug | |
| | ggacugucucugugcaucggcuuuuccacuuaaaucucu | |
| | uucacgcacaaguaucuuugugcacacucaucuauccua | |
| | accccguccacccucuucaagaccugcggcgauggggga | |
| | guggcgacgcaacagguggaggugaccacuggcaguugu | |
| | agucacgauccugcacguaggcggcccacggaccagugg | |
| | ucgcucggcccugugggcagcagggacguucggcagcau | |
| | ccugggcgacugagcagcccucucuagguuuacggaucc | |
| | gaauucucgacggaucgauccgaacaaacgacccaacac | |
| | ccgugcgugccgagagugaucccggcggcggucacgaac | |
| | uccagcaggaccaugugaucgcgcuucucguuggggucu | |
| | uugcucagggggacugggugcucagguagugguugucgg | |
| | gcagcagcacggggccgucgccgauggggguguucugcu | |
| | gguaguggucggcgagcugcacgcuucggccaugauaua | |
| | gacguuguggcuguuguaguuguacuccagcuugugccc | |
| | caggauguugccguccuccuugaagucgaugcccuucag | |
| | cucgaugcgguucaccagggugucgcccucgaacuucac | |
| | cucggggggucuuguaguugccgucguccuugaagaaga | |
| | uggugcgcuccuggacguagccuucgggcauggcggacu | |
| | ugaagaagucgugcugcuucauguggucgggguagcggc | |
| | ugaagcacugcacgccguaggucaggguggucacgaggg | |
| | ugggccagggcacgggcagcuugccgguggugcagauga | |
| | acuucagggucagcuugccguagguggcaucgcccucgc | |
| | ccucgccggacacgcugaacuuguggccguuuacgucgc | |
| | cguccagcucgaccaggaugggcaccaccccggugaaca | |
| | gcuccucgcccuugcucaccaugguggcuuuaccaacag | |
| | uaccggaaugccaagcuuggguccuguguucuggggcaa | |
| | acccguugcgaaaaagaacguucacggcgacuacugcac | |
| | uuauauacgguucucccccacccucgggaaaaaggcgga | |
| | gccaguacacgacaucacuuucccaguuuaccccgcgcc | |
| | accuucucuaggcaccggaucaauugccgaccccucccc | |
| | ccaacuucucggggacugugggcgaugugcgcucugccc | |
| | ugaaacugcacaaagacaauagucauucucgaucaccga | |
| | gagacuaccacu | |
| RNAIVT1200 | ggguguauggggugcucagggagggguaguaucucuggu | 15,011 |
| | auggagggcuugucgugcccuccuagggcagcucuccag | |
| | ccucugacccccaccugacacccagcucucacuuguggc | |
| | ucccaguagcugcuagcauguggcagcggccacaccccg | |
| | ggcaacggcuucgacaggccggcuaaaccuugugagggu | |
| | agccaucgggucaucgaccccuggugaaccagggcuuug | |
| | cucacccagcaugugaagacugcuucggcugaacagacg | |
| | gaagaaaccaauaagaagguucaacggcugagagggcga | |
| | cgcagcaaagcacuguggagugcuuagggcguguuggag | |
| | cacugccacuggacccaggcuuccaacgccgagagagug | |
| | ggacugucucugugcaucggcuuuuccacuuaaaucucu | |
| | uucacgcacaaguaucuuugugcacacucaucuauccua | |
| | accccguccacccucuucaagaccugcggcgauggggga | |
| | guggcgacgcaacagguggaggugaccacuggcaguugu | |
| | agucacgauccugcacguaggcggcccacggaccagugg | |
| | ucgcucggcccugugggcagcagggacguucggcagcau | |
| | ccugggcgacugagcagcccucucuagguuuacggaucc | |
| | gaauucucgacggaucgauccgaacaaacgacccaacac | |
| | ccgugcguuuuauucugucuuuuuauugccgaucccccg | |
| | gccgcuuuagcgggguggcaaggcuugcauaugcaacgc | |
| | aucauaaguguccuugguggcuguagagagacccuggua | |
| | cagaccguugccgagauucaauucauuguagagcugauu | |
| | cugacccugcugauaugcgggagcaucggcgcuucuaga | |
| | aaauuucacccuggagcgauaggcugcgaagucgcgugg | |
| | uggggcauagggcugguaaugcuugcgggugggcccggg | |
| | gcggcggggagucauguucauguagucacugugcaggag | |
| | ccugcuccucuuacuccuacaauacagagugaucacaag | |
| | ggagagaagcaacaccccacauguuccggccaaaggggc | |
| | ccaaauauaaauaucacacgcgaaguccaggccucgcgu | |
| | auggacggcgccuccugcugcgggccugcaagcuucagg | |
| | ucugaggcucaaugguuggcuggcuauaguuggaggggu | |
| | guggggggccuuggagcagguguuguggucuugaucucc | |
| | aguuugguccccugaccgaaaguccaggggacgugacug | |
| | gucucugcacaauaguagacgccgacguccucagccucg | |
| | acccgacuaaucuucagggugaaaucggcaccgcuaccu | |
| | gagccggaaaagcgauccgggaccccgcugaaacgguug | |
| | gaaaccuuguagaucaggagcuguggacuuuggcccggc | |
| | uucugcagauaccagugcagguagguguugccguuagag | |
| | uggaccagacucugggaacucuugcaggagaugcucgcc | |
| | ggcucaccaggggucacagacagagacaguggagucugg | |
| | gucaucacgauaucgcugccacccccuccagagccaccu | |
| | ccgccggagccaccgccaccggaagacacugucaccaug | |
| | gugcccuggccccagacgucgaaguaccagucauaauca | |
| | uacagggaggcgcagaaauacacggcgguaucuucggac | |
| | cugagacuagacagcuccauguaugccguagaacuugac | |
| | gugucgcgggucaugguaacccugccggugaacuucugg | |
| | uuguacucagaguuuccgcucgcaaaauagauccaaccc | |
| | auccacucgagucccugcccgggagccugccgcacccaa | |
| | uugauauaguaaucugggaagcuguagccggaagccuua | |
| | cagcucacuuucacguggcgccagguuuuuuaccuccgc | |
| | gccggacugaacaagcugcaccugcgggcgagcggcaug | |
| | caagagcagggcaagaggcagaagcagggcggucaccgg | |
| | cagagccaugguggcuuuaccaacaguaccggaaugcca | |
| | agcuugggucugggcucuuuuuugagcucggggagcaga | |
| | agcgcgcgaacagaagcgagaagcgaacugauugguuag | |
| | uucaaauaaggcacagggucauuucagguccuuggggca | |
| | cccuggaaacaucugaugguucucuagaaacugcugagg | |
| | ggggaccgcaucuggggaccaucuguucuuggcccugag | |
| | ccggggcaggaacugcuuaccacagauauccuguuccac | |
| | agauauccuguuuggcccauauucugugucucucuguuc | |
| | cuaaccuugaccuagcuugccaaaccuacaggugggguc | |
| | uuucauuccccccuuuuucuggagacuaaaaaaugaaac | |
| | ugcacaaagacaauagucauucucgaucaccgagagacu | |
| | accacu | |

Briefly, the RNAIVT381 template RNAs for use with the RNAIVT696 retrotransposon gene modifying polypeptide comprised the following sequences from 5′ to 3′: 5′ UTR, bGHpA, GFP, Kozak sequence, EF1a short promoter, 3′ UTR. The RNAIVT610 and RNAIVT1200 template RNAs for use with the RNAIVT1123 gene modifying polypeptide comprised the following sequences from 5′ to 3′: 5′ UTR, TKpA, GFP (RNAIVT610) or BCMA_CAR (RNAIVT1200), Kozak sequence, EF1a short promoter (RNAIVT610) or MND promoter (RNAIVT1200), 3′ UTR.

Activated primary human T cells were nucleofected with 1) the second retrotransposon gene modifying system containing a template RNA encoding either GFP or a CAR, 2) the second retrotransposon gene modifying system containing a template RNA encoding either GFP or a CAR and the first heterologous gene modifying system containing a template RNA designed to produce an insertion in TRAC or B2M, or 3) the second retrotransposon gene modifying system containing a template RNA encoding either GFP or a CAR and the first heterologous gene modifying system containing a template RNA designed to produce an insertion in TRAC and a template RNA designed to produce an insertion in B2M. The gene modifying systems were delivered in RNA format. Prior to nucleofection, T cells were cultured with TexMACS supplemented with 5% human AB Serum, IL2 at 20 IU/ml, IL7 at 10 ng/ml, IL15 at 5 ng/ml in each well at 37° C., 5% CO₂ for 2 days and stimulated using T Cell TransAct. For the nucleofection of the first heterologous gene modifying system, 1000 ng of heterologous gene modifying polypeptide encoding mRNA were combined with each of the 1000 ng template RNAs in RNA format. For the nucleofection of the second retrotransposon gene modifying system, either 800 ng (RNAIVT696 retrotransposon gene modifying polypeptide) or 6.25 ng (RNAIVT1123 retrotransposon gene modifying polypeptide) of mRNA encoding the retrotransposon gene modifying polypeptide were combined with 1000 ng of template RNA in RNA format.

The RNA mixture (comprising either the first heterologous gene modifying system or both the first heterologous and second retrotransposon gene modifying systems) was added to 500,000 primary human T cells in a total of 20 μL of Lonza P3 buffer and T cells were nucleofected in 16-well nucleofection cassettes using program DS-130. After nucleofection, cells incubated at room temperature for 10 minutes and were transferred to 24-well plates containing 500 μL of TexMACS supplemented with 5% human AB Serum, IL2 at 20 IU/ml, IL7 at 10 ng/ml, IL15 at 5 ng/ml in each well and cultured at 37° C., 5% CO₂ for 4 days prior to flow cytometry analysis.

To determine whether insertion by the second retrotransposon gene modifying system was successful, flow cytometry was used to determine the fraction of cells expressing GFP or BCMA CAR as detected by staining cells with fluorescently labeled Recombinant Human BCMA Protein. To analyze cell surface markers representative of different T cells subpopulations, cells were stained with fluorescently labeled anti human CD3, CD8, CD62L, CD45RA antibodies. To determine whether editing of the TRAC locus by the first heterologous gene modifying system was successful, CD3 levels were measured by flow cytometry as a surrogate readout. Functional loss of TRAC is known to be associated with loss of surface expression of the CD3 complex. To determine whether editing of the B2M locus by the first heterologous gene modifying system was successful, expression of B2M was measured by flow cytometry.

To measure T cell effector functions, expression of TNFa, IL2 and IFNy cytokines was measured upon stimulation. Specifically, edited T cells were stimulated for 6 hours with PMA (50 ng/ml) and Ionomycin (1 μg/ml) in presence of 1 μl/ml of Golgi Transport Inhibitor. Cells were then stained for surface antigens, fixed and permeabilized using Cytofix/Cytoperm Kit (BD) according to the manufacturer's instruction. After fixation and permeabilization, cells were stained using antibodies recognizing human TNFa, IL2, and IFNy cytokines.

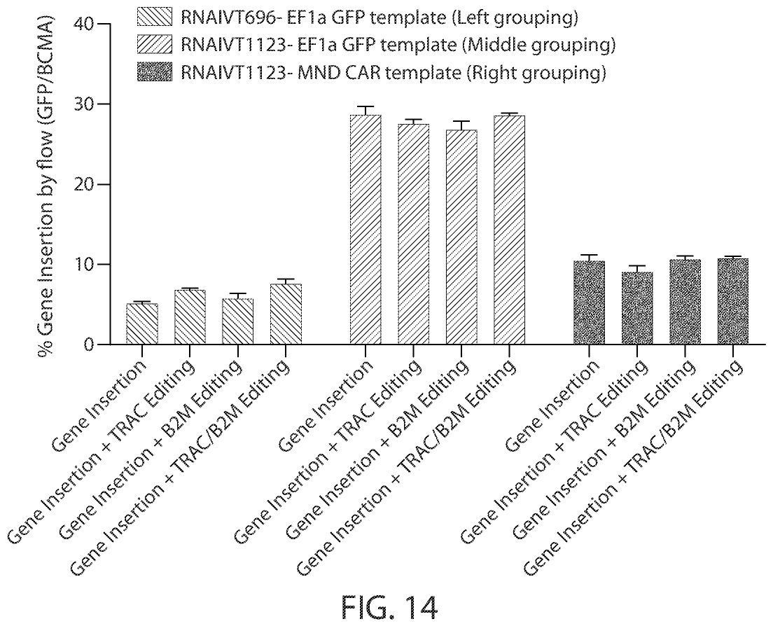

FIG. 14 shows the levels of editing by the second retrotransposon gene modifying system in the bulk T cell population when the cells were transfected with the second retrotransposon gene modifying system, either alone or in combination with the first heterologous gene modifying system. The results show an average of 5.1% T cells expressing GFP when transfected with RNAIVT696 retrotransposon gene modifying polypeptide and EF1A GFP template RNA, an average of 28.8% T cells expressing GFP when transfected with RNAIVT1123 retrotransposon gene modifying polypeptide and EF1a GFP template RNA, and an average of 10.5% T cells expressing BCMA CAR when transfected with RNAIVT1123 retrotransposon gene modifying polypeptide and MND CAR template RNA. When these 3 configurations of the second retrotransposon gene modifying system were delivered in combination with the first heterologous gene modifying system (targeting TRAC, B2M, or both loci), average levels of T cells expressing GFP or CAR were not significantly changed. For example, in the condition where both TRAC and B2M are edited, the results show an average of 7.6% T cells expressing GFP for RNAIVT696+EF1A GFP template RNA, an average of ~28% T cells expressing GFP for RNAIVT1123+EF1a GFP template RNA, and an average of 10.9% T cells expressing BCMA CAR for RNAIVT1123+MND CAR template RNA. The results showed that gene modifying systems inserting whole genes can function in combination with gene modifying systems directing other edits (e.g., short insertions). The results further showed that editing efficiency when inserting whole genes using the second retrotransposon gene modifying system is not affected by the co-delivery of another exemplary gene modifying system containing a heterologous gene modifying polypeptide which directs one or multiple edits (e.g. short insertions).

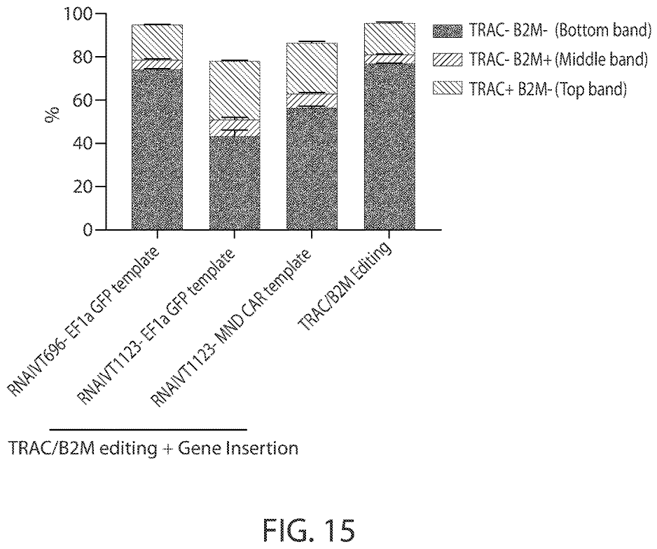

FIG. 15 shows the levels of editing by the first heterologous gene modifying system (containing a template RNA designed to produce an insertion in TRAC and a template RNA designed to produce an insertion in B2M) in the bulk T cell population when the cells were transfected with the first heterologous gene modifying system either alone or in combination with the second retrotransposon gene modifying system. The results showed an average of 95.8% of cells were edited at one or both of the TRAC or B2M loci when treated with the first heterologous gene modifying system as measured by flow cytometry. Specifically, 77% of cells showed editing in both TRAC (measured by lack of expression of CD3) and B2M, 4.3% of cells showed editing only in TRAC and 14.5% of cells showed editing only in B2M. When the first heterologous gene modifying system was delivered in combination with the second retrotransposon gene modifying system, levels of CD3 negative T cells were measured in the range of 94.9 to 78.21% and the levels of editing of both TRAC and B2M in the range of 74.2 to 46.3%. The results showed that gene modifying systems using an exemplary heterologous gene modifying polypeptide successfully disrupted both the TRAC and B2M genes in T cells. These results further show that heterologous gene modifying systems produce edits sufficient to knock out gene function when used in combination with an additional retrotransposon gene modifying systems designed to produce whole gene insertions. The results show that editing efficiency when making multiple short edits (e.g., multiple 5 nucleotide insertions) is not substantially affected by the co-delivery of another exemplary gene modifying system containing a retrotransposon gene modifying polypeptide (e.g., directing a insertion of a gene).

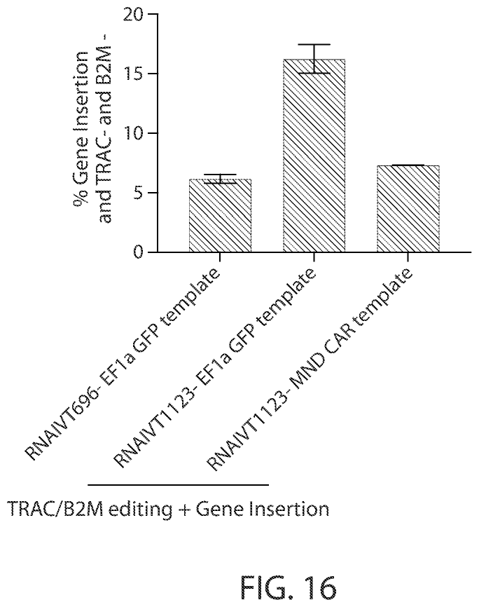

FIG. 16 shows the levels of cells edited by the first heterologous gene modifying system (measured here as combined loss of expression of CD3 and B2M by flow cytometry) and also edited by the second retrotransposon gene editing system (that were GFP+ or BCMA CAR+ T cells). The results demonstrated that treating T cells with both the first and second gene modifying systems results in substantial levels of co-occurrence of the three editing events in the same cells. For example, the results showed an average of ~6% T cells lacking TRAC and B2M and also expressing GFP for RNAIVT696+EF1A GFP template RNA, an average of ~16% T cells lacking TRAC and B2M and also expressing GFP for RNAIVT1123+EF1a GFP template RNA, and an average of ~7% T cells lacking TRAC and B2M and also expressing BCMA CAR for RNAIVT1123+MND CAR template RNA.

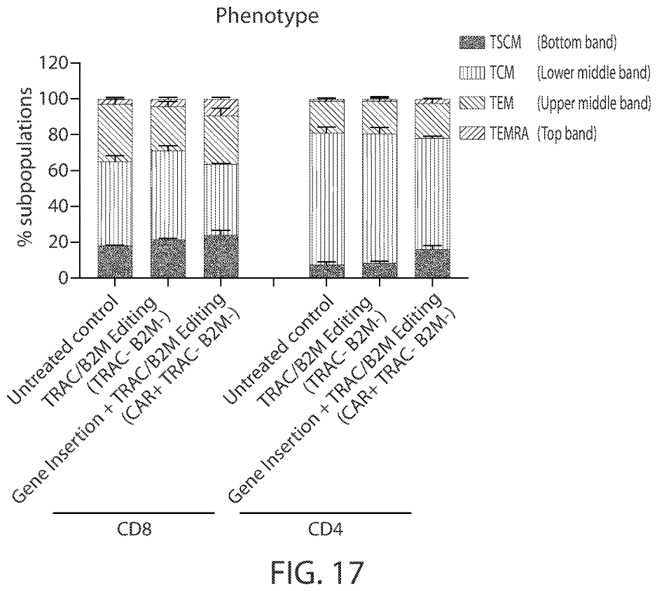

FIG. 17 shows a phenotypic characterization by flow cytometry of T cells treated with the first heterologous gene modifying system (containing a template RNA designed to produce an insertion in TRAC and a template RNA designed to produce an insertion in B2M), both the first heterologous and second retrotransposon gene modifying systems, or a mock treatment lacking both gene modifying systems. Without wishing to be bound by theory, following stimulation, T cells progressively differentiate into memory and terminally differentiated effectors, acquiring effector functions and losing the ability for self-renewal and survival, and concomitant cell surface marker expression changes can be used to monitor said transitions. Stem memory T cells (TSCM) are defined as CD62L+CD45RA+, Central memory (CM) are defined as CD62L+CD45RA−, Effector memory (TEM) are defined as CD62L-CD45RA−, Terminal effectors (TEMRA) are defined as CD62L-CD45RA+. The distribution of the indicated T cells subpopulations within CD8 and CD4 compartments, comparing mock treated cells, CD3-B2M-cells edited with the first heterologous gene modifying system and CAR+CD3-B2M-cells edited by both the first heterologous and the second retrotransposon gene modifying systems, were graphed in FIG. 17. The results showed no substantial skewing in the distribution of the TSCM, CM, TEM, and TEMRA subpopulations when comparing mock treated cells, cells edited by just the first heterologous gene modifying system comprising both TRAC and B2M edits, and cells edited by both the first heterologous and second retrotransposon gene modifying systems. These results demonstrated that co-occurrence of three editing events (e.g., two by the first heterologous gene modifying system and one by the second retrotransposon gene modifying system) does not impact the differentiation phenotypes of primary human T cells.

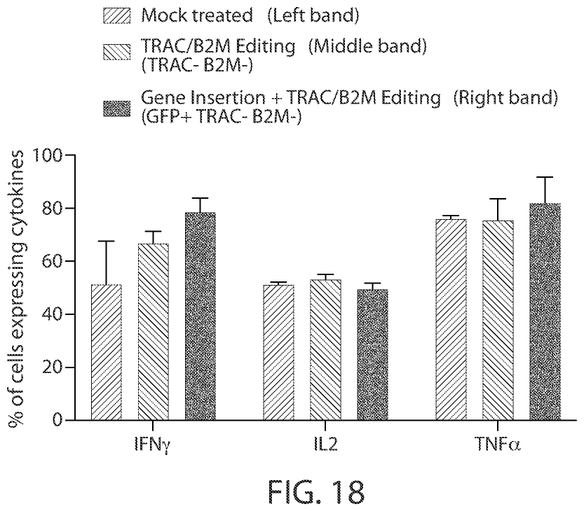

FIG. 18 shows a functional characterization by flow cytometry of T cells treated with the first heterologous gene modifying system (containing a template RNA designed to produce an insertion in TRAC and a template RNA designed to produce an insertion in B2M), both the first heterologous and second retrotransposon gene modifying systems, or a mock treatment lacking both gene modifying systems. Expression of cytokines (such as TNFa, IL2, IFNy) is a prerequisite for T cells to perform effector functions. The results showed no substantial alteration in the fraction of T cells capable of secreting the indicated cytokines when comparing mock treated cells, cells edited by just the first heterologous gene modifying system comprising both edits, and cells edited by both the first heterologous and second retrotransposon gene modifying systems. These results demonstrated that co-occurrence of three editing events (e.g., two by the first heterologous gene modifying system and one by the second retrotransposon gene modifying system) does not impact the basal effector functions of primary human T cells.

### Example 12: LNP Delivery of Gene Modifying Systems for Multiplex Editing in T Cells

LNPs were used to deliver two gene modifying systems to effect multiplex editing of the genomic DNA of activated primary human T cells.

LNPs were formulated with a first exemplary heterologous gene modifying system containing an mRNA encoding a first heterologous gene modifying polypeptide and 2 template RNAs to produce an insertion of 5 nucleotides (TAGTG) in the first exon of the human TRAC gene and an insertion of 5 nucleotides (TAGTG) in the first exon of the Beta 2 Microglobulin (B2M) gene in human T cells. The insertions into TRAC and B2M generated a frameshift that resulted in a non-functional gene (also referred to as a knock-out). LNPs were also formulated with a second exemplary retrotransposon gene modifying system containing an mRNA encoding a retrotransposon gene modifying polypeptide and a template RNA to insert expression cassettes for a chimeric antigen receptor (CAR) or GFP into the genome of the human T cells.

The template RNA of the first heterologous gene modifying system contained:
1. a gRNA spacer;
2. a gRNA scaffold;
3. a heterologous object sequence; and
4. a primer binding site (PBS) sequence.

The exemplary template RNAs generated and used in the first heterologous gene modifying system are given in Table E18. Nucleotide modifications are noted as follows: phosphorothioate linkages denoted by an asterisk, 2′-O-methyl groups denoted by an ‘m’ preceding a nucleotide.

Exemplary Template RNAs and Sequences

| | | | SEQ |
|---|---|---|---|
| Name | Descriptor | Sequence | ID NO |
| RNACS5630 | TRAC4_ | mG*mA*mG*rArArUrCrAr | 18201 |
| | Ins_5 | ArArArUrCrGrGrUrGrAr | |
| | Nick_ | ArUrGrUrUrUrUrArGrAm | |
| | P9R18 | GmCmUmAmGmAmAmAmUmAm | |
| | | GmCrArArGrUrUrArArAr | |
| | | ArUrArArGrGrCrUrArGr | |
| | | UrCrCrGrUrUrArUrCrAm | |
| | | AmCmUmUmGmAmAmAmAmAm | |
| | | GmUmGmGmCmAmCmCmGmAm | |
| | | GmUmCmGmGmUmGmCrCrUr | |
| | | GrUrCrUrGrCrCrUrArUr | |
| | | UrUrArGrUrGrCrArCrCr | |
| | | GrA*mU*mU*mU | |
| RNACS7755 | B2m1_ | mA*mC*mU*rCrArCrGrCr | 18202 |
| | P11R18_ | UrGrGrArUrArGrCrCrUr | |
| | ins5_ | CrCrGrUrUrUrUrArGrAm | |
| | nick_ | GmCmUmAmGmAmAmAmUmAm | |
| | v1 | GmCrArArGrUrUrArArAr | |
| | | ArUrArArGrGrCrUrArGr | |
| | | UrCrCrGrUrUrArUrCrAm | |
| | | AmCmUmUmGmAmAmAmAmAm | |
| | | GmUmGmGmCmAmCmCmGmAm | |
| | | GmUmCmGmGmUmGmCrUrUr | |
| | | UrCrUrGrGrCrCrUrGrGr | |
| | | ArUrGrArUrGrGrGrCrUr | |
| | | ArUrCrC*mA*mG*mC | |

Table E18A shows the sequences of Table E18 without chemical modifications. In some embodiments, the sequences of Table E18A may be used without chemical modifications, or with one or more chemical modifications.

TABLE E18A
Table E18 Sequences without Chemical
Modifications

| | | | SEQ |
|---|---|---|---|
| | | | ID |
| RNACS | Name | Sequence | NO |
| RNACS5630 | TRAC4_ | GAGAAUCAAAAUCGGUGAAU | 18203 |
| | Ins5 | GUUUUAGAGCUAGAAAUAGC | |
| | Nick | AAGUUAAAAUAAGGCUAGUC | |
| | P9R18 | CGUUAUCAACUUGAAAAAGU | |
| | | GGCACCGAGUCGGUGCCUGU | |
| | | CUGCCUAUUUAGUGCACCGA | |
| | | UUU | |
| RNACS7755 | B2m1_ | ACUCACGCUGGAUAGCCUCC | 18204 |
| | P11R18_ | GUUUUAGAGCUAGAAAUAGC | |
| | ins5_ | AAGUUAAAAUAAGGCUAGUC | |
| | nick_ | CGUUAUCAACUUGAAAAAGU | |
| | v1 | GGCACCGAGUCGGUGCUUUC | |
| | | UGGCCUGGAUGAUGGGCUAU | |
| | | CCAGC | |

Exemplary heterologous gene modifying polypeptides of the first heterologous gene modifying system contained:
1. an endonuclease and/or DNA binding domain;
2. a peptide linker; and
3. a reverse transcriptase (RT) domain,
   where (1), (2), and (3) are heterologous to each other. Accordingly, the first heterologous gene modifying polypeptides were examples of heterologous gene modifying polypeptides.

The exemplary gene heterologous modifying polypeptide of the first gene modifying system was RNAIVT1564, and comprised the amino acid sequence indicated in Table E19.

TABLE E19
Exemplary heterologous gene modifying polypeptide and Sequence

| Name | AA sequence | SEQ ID NO |
|---|---|---|
| RNAIVT1564 | MPAAKRVKLDGGDKKYSIGLDIGTNSVGWAVITDEYKVPSK | 15,008 |
| | KFKVLGNTDRHSIKKNLIGALLFDSGETAEATRLKRTARRRY | |
| | TRRKNRICYLQEIFSNEMAKVDDSFFHRLEESFLVEEDKKHER | |
| | HPIFGNIVDEVAYHEKYPTIYHLRKKLVDSTDKADLRLIYLAL | |
| | AHMIKFRGHFLIEGDLNPDNSDVDKLFIQLVQTYNQLFEENPI | |
| | NASGVDAKAILSARLSKSRRLENLIAQLPGEKKNGLFGNLIAL | |
| | SLGLTPNFKSNFDLAEDAKLQLSKDTYDDDLDNLLAQIGDQY | |
| | ADLFLAAKNLSDAILLSDILRVNTEITKAPLSASMIKRYDEHH | |
| | QDLTLLKALVRQQLPEKYKEIFFDQSKNGYAGYIDGGASQEE | |
| | FYKFIKPILEKMDGTEELLVKLNREDLLRKQRTFDNGSIPHQI | |
| | HLGELHAILRRQEDFYPFLKDNREKIEKILTFRIPYYVGPLARG | |
| | NSRFAWMTRKSEETITPWNFEEVVDKGASAQSFIERMTNFDK | |
| | NLPNEKVLPKHSLLYEYFTVYNELTKVKYVTEGMRKPAFLSG | |
| | EQKKAIVDLLFKTNRKVTVKQLKEDYFKKIECFDSVEISGVED | |
| | RFNASLGTYHDLLKIIKDKDFLDNEENEDILEDIVLTLTLFEDR | |
| | EMIEERLKTYAHLFDDKVMKQLKRRRYTGWGRLSRKLINGI | |
| | RDKQSGKTILDFLKSDGFANRNFMQLIHDDSLTFKEDIQKAQ | |
| | VSGQGDSLHEHIANLAGSPAIKKGILQTVKVVDELVKVMGRH | |
| | KPENIVIEMARENQTTQKGQKNSRERMKRIEEGIKELGSQILK | |
| | EHPVENTQLQNEKLYLYYLQNGRDMYVDQELDINRLSDYDV | |
| | DHIVPQSFLKDDSIDNKVLTRSDKARGKSDNVPSEEVVKKMK | |
| | NYWRQLLNAKLITQRKFDNLTKAERGGLSELDKAGFIKRQLV | |
| | ETRQITKHVAQILDSRMNTKYDENDKLIREVKVITLKSKLVSD | |
| | FRKDFQFYKVREINNYHHAHDAYLNAVVGTALIKKYPKLESE | |
| | FVYGDYKVYDVRKMIAKSEQEIGKATAKYFFYSNIMNFFKTE | |
| | ITLANGEIRKRPLIETNGETGEIVWDKGRDFATVRKVLSMPQV | |
| | NIVKKTEVQTGGFSKESILPKRNSDKLIARKKDWDPKKYGGF | |
| | DSPTVAYSVLVVAKVEKGKSKKLKSVKELLGITIMERSSFEK | |
| | NPIDFLEAKGYKEVKKDLIIKLPKYSLFELENGRKRMLASAGE | |
| | LQKGNELALPSKYVNFLYLASHYEKLKGSPEDNEQKQLFVEQ | |
| | HKHYLDEIIEQISEFSKRVILADANLDKVLSAYNKHRDKPIRE | |
| | QAENIIHLFTLTNLGAPAAFKYFDTTIDRKRYTSTKEVLDATLI | |
| | HQSITGLYETRIDLSQLGGDSGGSSGGSSGSETPGTSESATPES | |
| | SGGSSGGSSTLNIEDEYRLHETSKEPDVSLGSTWLSDFPQAWA | |
| | ETGGMGLAVRQAPLIIPLKATSTPVSIKQYPMSQEARLGIKPHI | |
| | QRLLDQGILVPCQSPWNTPLLPVKKPGTNDYRPVQDLREVNK | |
| | RVEDIHPTVPNPYNLLSGLPPSHQWYTVLDLKDAFFCLRLHPT | |
| | SQPLFAFEWRDPEMGISGQLTWTRLPQGFKNSPTLFNEALHR | |
| | DLADFRIQHPDLILLQYVDDLLLAATSELDCQQGTRALLQTL | |
| | GNLGYRASAKKAQICQKQVKYLGYLLKEGQRWLTEARKET | |
| | VMGQPTPKTPRQLREFLGKAGFCRLFIPGFAEMAAPLYPLTKP | |
| | GTLFNWGPDQQKAYQEIKQALLTAPALGLPDLTKPFELFVDE | |
| | KQGYAKGVLTQKLGPWRRPVAYLSKKLDPVAAGWPPCLRM | |
| | VAAIAVLTKDAGKLTMGQPLVILAPHAVEALVKQPPDRWLS | |
| | NARMTHYQALLLDTDRVQFGPVVALNPATLLPLPEEGLQHN | |
| | CLDILAEAHGTRPDLTDQPLPDADHTWYTDGSSLLQEGQRKA | |
| | GAAVTTETEVIWAKALPAGTSAQRAELIALTQALKMAEGKK | |
| | LNVYTDSRYAFATAHIHGEIYRRRGWLTSEGKEIKNKDEILAL | |
| | LKALFLPKRLSIIHCPGHQKGHSAEARGNRMADQAARKAAIT | |
| | ETPDTSTLLIENSSPSGGSKRTADGSEFEKRTADGSEFESPKKK | |
| | AKVE | |

Exemplary gene modifying polypeptides of the second retrotransposon gene modifying system contained:
1. an endonuclease and/or DNA binding domain; and
2. a reverse transcriptase (RT) domain,
   where (1) and (2) are both derived from a retrotransposon (from the same retrotransposons). Accordingly, the second gene modifying polypeptide was an example of retrotransposon gene modifying polypeptides.

The second retrotransposon gene modifying system used comprised the amino acid sequence in Table E20. The mRNA sequence encoding the second retrotransposon gene modifying system is also given in Table E20.

TABLE E20
Exemplary gene modifying polypeptides and sequences

| | | SEQ | | SEQ |
|---|---|---|---|---|
| | | ID | | ID |
| Name | Nucleic Acid Sequence | NO | Amino Acid Sequence | NO |
| RTE- | AGGAAAUAAGAGAGAAAAGAAGAGUAA | 18205 | MPKKKRKVGGSDSTAHPNQGRGLEKVSQ | 18206 |
| 1_MD | GAAGAAAUAUAAGAGCCACCAUGCCAA | | TLPALQTPGQHTAAGGSSPLSGRNQRKN | |
| RNAIV | AGAAGAAAAGAAAAGUAGGAGGUUCCG | | TKKLLLGAWNIRTLLDRENTPRPERRTA | |
| T1901 | ACUCCACAGCUCAUCCCAACCAGGGAC | | LIGKELARYNIDIAALSETRLPEEGSLS | |
| | GUGGCCUGGAGAAGGUGUCCCAGACUC | | EPTTGYTFFWKGRASNEDRIHGVGLAI | |
| | UGCCUGCGCUGCAGACCCCAGGGCAGC | | KTSLLKQLPDLPVGISERLMKIRLPLS | |
| | ACACUGCCGCUGGGGGAUCCUCUCCCC | | KDRYATIISAYAPTLTSTEETIEQFYS | |
| | UGUCCGGCAGGAAUCAGCGGAAGAACA | | DLSAVLHSVPTNDKLILLGDFNARVGQ | |
| | CCAAAAAGCUGUUGCUGGGCGCCUGGA | | DHERWKGVLGKHGVGKMNNNGLLLLSK | |
| | ACAUCCGCACCCUGCUCGACCGCGAAA | | CSEFELTITNTVFRMANKYKTTWMHPR | |
| | AUACUCCCCGUCCUGAAAGACGCACGG | | SKQWHLIDYIIVRRRDIQDVKITRAMR | |
| | CACUGAUUGGGAAAGAGCUCGCCAGAU | | GAECWTDHRLVRATLQMRIAPRHPKRA | |
| | AUAACAUCGACAUCGCUGCCCUGUCCG | | QTVRAFYNVSRLRDPSYLQTFQSCLDD | |
| | AAACCCGCCUGCCAGAGGAAGGAUCUC | | KLSAKGPLTGSSTEKWNQFRDAVKETS | |
| | UGAGCGAGCCUACCACUGGCUACACGU | | KAVLGPKQRNHQDWFDENNTAIEDLLS | |
| | UCUUUUGGAAGGGCAGAGCAAGCAACG | | KKNKAFMEWQNNPNSAPKKDRFKSLQA | |
| | AGGACCGUAUCCAUGGUGUAGGCCUGG | | TAQREIRKMQDRWWEKKAEEIQRFADM | |
| | CUAUCAAGACCUCUCUCCUGAAACAAC | | KNYKQFFSALKTVYGPLKPTTTPLLSS | |
| | UCCCAGAUCUGCCAGUCGGCAUCUCAG | | DGDTLIKDKKGISNRWKEHFSQLLNRP | |
| | AGAGACUGAUGAAGAUCAGGCUUCCUC | | SSVDQSALDQIPQNRTIEQLDVPPSIE | |
| | UCUCAAAAGACCGCUACGCAACCAUCA | | EVQKAIKQMSAGKAPGKDGIPTEVYKA | |
| | UUUCCGCAUACGCGCCGACCCUGACUA | | LNGKALQAFHIVLTSIWEEEDMPPELR | |
| | GCACCGAGGAAACCAUUGAGCAGUUCU | | DASIVALYKNKGSRAACDNYRGISLLS | |
| | ACUCUGAUCUUUCAGCUGUUUUGCACU | | TAGKILARVILNRLLSSVSEQNLPESQ | |
| | CCGUGCCUACCAACGACAAGCUGAUCC | | CGFRPDRSTIDMVFTVRQMQEKCLEQN | |
| | UCCUGGGCGACUUCAACGCUCGCGUGG | | LSLYIVFIDLTKAFDTVNRDALWVILS | |
| | GUCAGGACCAUGAGCGCUGGAAAGGCG | | KLGCPAKFVKLIQLFHVDMTGEVLSGG | |
| | UGCUGGGUAAGCAUGGCGUGGGUAAGA | | ETSDRFNISNGVKQGCVLAPVLFNLFF | |
| | UGAACAAUAACGGCCUCCUGCUCCUGU | | TQVLRHAVMDLDLGVYIKYRLDGSLFD | |
| | CUAAGUGUAGCGAAUUCGAGCUGACUA | | LRRLTAKTKTTERLILEALFADDCALM | |
| | UUACCAACACUGUUUUUCGUAUGGCUA | | AHQENHLQTIVDRFSTATKLFGLTISL | |
| | ACAAGUACAAAACUACAUGGAUGCACC | | SKTEVLFQPAPGRPTNQPCITIDGTQL | |
| | CCAGGAGCAAGCAGUGGCACCUGAUUG | | SNVNTFKYLGSTIANDGSLDHEINARI | |
| | ACUACAUUAUCGUGCGUAGGCGGGAUA | | QKASQALGRLRCKVLQHRGVSTATKLK | |
| | UCCAGGACGUGAAGAUUACCCGUGCAA | | VYNAVVLSSLLYGCETWTLYRKHMKQL | |
| | UGCGCGGCGCUGAGUGCUGGACAGACC | | EQFHQRSLRSIMRIRWQDRITNQEVLD | |
| | AUCGCCUGGUGAGAGCUACUCUCCAGA | | RANSTSIEVMVLKTQLRWSGHVIRMDP | |
| | UGAGGAUCGCACCGCGUCACCCUAAGC | | QRIPRQVFYGELSAGLRKQGRPKKRFK | |
| | GCGCCCAGACCGUGCGGGCCUUUUACA | | DQLKSNLKWAGITPKQLELAASDRSSW | |
| | ACGUAUCACGGCUUCGGGAUCCUUCCU | | RTHINHAATTFEDERRRRLAAARERRH | |
| | ACCUUCAGACAUUUCAAUCUUGCCUCG | | QATTAPPVTTGVPCPMCHKLCASAFGL | |
| | AUGACAAGCUCUCUGCUAAGGGUCCCC | | QSHMRVHRRSGSETPGTSESATPESVS | |
| | UCACCGGAUCUUCAACAGAGAAGUGGA | | GWRLFKKIS | |
| | ACCAAUUCCGCGAUGCGGUGAAGGAAA | | | |
| | CCAGCAAAGCAGUCCUGGGACCGAAGC | | | |
| | AGCGCAACCACCAAGACUGGUUUGACG | | | |
| | AAAAUAACACUGCCAUCGAAGACCUCC | | | |
| | UGUCUAAAAAGAACAAAGCAUUUAUGG | | | |
| | AGUGGCAGAAUAACCCAAACAGUGCAC | | | |
| | CCAAGAAAGACCGUUUCAAAAGCCUGC | | | |
| | AGGCCACCGCGCAGCGCGAGAUCCGGA | | | |
| | AGAUGCAGGACCGCUGGUGGGAAAAAA | | | |
| | AGGCCGAAGAGAUCCAGAGGUUCGCGG | | | |
| | AUAUGAAAAACUACAAGCAGUUCUUUA | | | |
| | GCGCACUGAAGACCGUGUACGGACCUC | | | |
| | UCAAACCCACCACGACUCCCCUGCUCU | | | |
| | CUUCCGAUGGUGACACUCUCAUCAAGG | | | |
| | ACAAAAAGGGAAUCAGCAACCGCUGGA | | | |
| | AAGAACACUUUUCUCAACUUCUCAACC | | | |
| | GCCCAAGCUCCGUGGACCAGAGCGCUC | | | |
| | UGGACCAGAUCCCGCAGAACCGUACCA | | | |
| | UCGAGCAGCUGGACGUGCCUCCCUCCA | | | |
| | UCGAAGAGGUCCAGAAGGCUAUCAAGC | | | |
| | AGAUGUCUGCUGGGAAGGCUCCUGGCA | | | |
| | AGGAUGGGAUCCCAACAGAGGUCUAUA | | | |
| | AAGCUCUCAACGGAAAGGCGCUGCAGG | | | |
| | CCUUUCACAUUGUCCUGACCUCCAUCU | | | |
| | GGGAGGAAGAGGACAUGCCACCUGAAC | | | |
| | UUAGGGACGCCUCUAUUGUGGCCCUGU | | | |
| | ACAAGAAUAAGGGCAGCCGCGCAGCUU | | | |
| | GUGAUAACUAUCGUGGCAUCUCACUCC | | | |
| | UGAGUACAGCUGGCAAGAUUCUGGCUC | | | |
| | GUGUGAUCCUGAACCGGUUGCUGAGUU | | | |
| | CCGUUUCCGAGCAGAACCUGCCCGAAU | | | |
| | CACAGUGCGGCUUCCGCCCCGAUCGGU | | | |
| | CCACCAUCGAUAUGGUGUUCACCGUGC | | | |
| | GUCAGAUGCAGGAGAAGUGCCUGGAAC | | | |
| | AGAAUCUGAGCCUGUACAUCGUUUUCA | | | |
| | UCGAUCUGACUAAGGCCUUCGACACGG | | | |
| | UGAACCGCGAUGCCCUGUGGGUGAUCC | | | |
| | UGAGCAAGCUGGGGUGCCCCGCUAAGU | | | |
| | UCGUAAAGCUCAUCCAGUUGUUCCACG | | | |
| | UUGAUAUGACGGGAGAGGUCCUGUCCG | | | |
| | GUGGAGAAACCUCCGACCGGUUUAACA | | | |
| | UCUCCAAUGGCGUGAAACAGGGCUGCG | | | |
| | UCCUGGCCCCAGUGCUGUUCAACCUGU | | | |
| | UUUUCACACAGGUGCUGAGACAUGCGG | | | |
| | UUAUGGAUCUUGACCUGGGUGUGUAUA | | | |
| | UCAAGUAUCGCUUGGACGGCAGUCUGU | | | |
| | UCGAUCUCAGACGCCUGACAGCUAAGA | | | |
| | CUAAGACUACCGAGCGCCUCAUUCUCG | | | |
| | AAGCCCUGUUUGCCGACGAUUGUGCCC | | | |
| | UUAUGGCCCACCAGGAGAACCACCUGC | | | |
| | AGACGAUUGUCGACCGCUUCUCUACAG | | | |
| | CGACCAAGCUGUUCGGCCUCACCAUCU | | | |
| | CCCUCUCAAAGACCGAAGUCCUGUUUC | | | |
| | AGCCCGCACCCGGGAGACCCACGAAUC | | | |
| | AGCCGUGCAUUACCAUUGACGGGACCC | | | |
| | AACUUUCCAACGUCAACACCUUCAAGU | | | |
| | AUCUGGGAUCUACCAUCGCCAAUGAUG | | | |
| | GCAGCCUGGAUCAUGAAAUCAACGCAA | | | |
| | GGAUCCAAAAGGCUAGCCAGGCACUGG | | | |
| | GCCGCCUCCGGUGCAAGGUCCUGCAGC | | | |
| | ACCGGGGCGUUUCCACUGCCACUAAAC | | | |
| | UGAAGGUGUACAACGCUGUCGUGCUCU | | | |
| | CUUCCCUGCUUUAUGGCUGCGAAACAU | | | |
| | GGACUCUGUACCGGAAGCAUAUGAAAC | | | |
| | AGCUUGAGCAGUUCCACCAGAGGAGCU | | | |
| | UGCGUAGCAUCAUGAGGAUCCGCUGGC | | | |
| | AGGACCGCAUCACCAACCAGGAGGUGC | | | |
| | UGGACAGGGCUAAUUCCACCAGCAUCG | | | |
| | AAGUUAUGGUGCUGAAGACCCAGCUCC | | | |
| | GUUGGUCUGGCCAUGUCAUUCGGAUGG | | | |
| | ACCCUCAGCGUAUCCCCCGCCAGGUAU | | | |
| | UCUACGGGGAACUGUCUGCGGGGCUCA | | | |
| | GAAAGCAGGGCCGCCCAAAGAAACGCU | | | |
| | UUAAGGAUCAGCUGAAAAGCAAUCUGA | | | |
| | AGUGGGCCGGCAUCACCCCCAAGCAGC | | | |
| | UGGAGCUGGCGGCAUCUGACCGCUCCA | | | |
| | GCUGGCGCACCCACAUUAACCACGCAG | | | |
| | CUACUACCUUUGAGGACGAACGCCGGC | | | |
| | GCAGACUGGCCGCUGCACGCGAGAGGC | | | |
| | GCCACCAAGCCACUACCGCCCCUCCCG | | | |
| | UGACCACUGGGGUCCCUUGCCCUAUGU | | | |
| | GCCACAAGCUGUGUGCGUCUGCCUUCG | | | |
| | GUUUGCAGUCCCACAUGCGUGUGCAUC | | | |
| | GCCGCAGCGGCAGCGAGACUCCCGGGA | | | |
| | CCUCAGAGUCCGCCACACCCGAAAGUG | | | |
| | UGAGCGGCUGGCGGCUGUUCAAGAAGA | | | |
| | UUAGCUGAGCUGGAGCCUCGGUGGCCA | | | |
| | UGCUUCUUGCCCCUUGGGCCUCCCCCC | | | |
| | AGCCCCUCCUCCCCUUCCUGCACCCGU | | | |
| | ACCCCCGUGGUCUUUGAAUAAAGUCUG | | | |
| | AAAAAAAAAAAAAAAAAAAAAAAAAAA | | | |
| | AAAAAAAAAAAAAAAAAAAAAAAAAAA | | | |
| | AAAAAAAAAAAAAAAAAAAAAAAAAAA | | | |

The template RNA for use with the retrotransposon gene modifying polypeptide comprised the following sequences from 5′ to 3′: either 5′UTRᵣₑₜᵣₒ, BCMA CAR, MND promoter, 3′UTRᵣₑₜᵣₒ or 5′UTRᵣₑₜᵣₒ, GFP, MND promoter, 3′UTRᵣₑₜᵣₒ

The template RNAs comprised an RTE1_MD 5′UTR having the sequence of:

(SEQ ID NO: 37653)
GGGUGUAUGGGGUGCUCAGGGAGGGGUAGUAUCUCUGGUAUGGAGG
GCUUGUCGUGCCCUCCUAGGGCAGCUCUCCAGCCUCUGACCCCCA
CCUGACACCCAGCUCUCACUUGUGGCUCCCAGUAGCUGCUAGCAU
GUGGCAGCGGCCACACCCCGGGCAACGGCUUCGACAGGCCGGCUA
AACCUUGUGAGGGUAGCCAUCGGGUCAUCGACCCCUGGUGAACCA
GGGCUUUGCUCACCCAGCAUGUGAAGACUGCUUCGGCUGAACAGA
CGGAAGAAACCAAUAAGAAGGUUCAACGGCUGAGAGGGCGACGCA
GCAAAGCACUGUGGAGUGCUUAGGGCGUGUUGGAGCACAAAGGAC
AACACGGCCAUCCAAUGCAGCUGAGGAAGUCUCCAGAUGUAACAA
UUUUUCGUGCCACUGGACCCAGGCUUCCAACGCCGAGAGAGUGGG
ACUGUCUCUGUGCAUCGGCUUUUCCACUUAAAUCUCUUUCACGCA
CAAGUAUCUUUGUGCACACUCAUCUAUCCUAACCCCGUCCACCCU
CUUCAAGACCUGCGGCGAUGGGGGAGUGGCGACGCAACAGGUGGA
GGUGACCACUGGCAGUUGUAGUCACGAUCCUGCACGUAGGCGGCC
CACGGACCAGUGGUCGCUCGGCCCUGUGGGCAGCAGGGACGUUCG
GCAGCAUCCUGGGCGACUGAGCAGCCCUCUCUAG

The template RNAs comprised an RTE1_MD 3′UTR having the sequence of:

(SEQ ID NO: 18207)
UGAAACUGCACAAAGACAAUAGUCAUUCUCGAUCACCGAGAGAC
UACCACU

The CAR template RNA comprised a nucleic acid sequence encoding a BCMA-CAR fusion polypeptide, wherein the nucleic acid sequence was:

(SEQ ID NO: 18208)
AGGGGUGUAUGGGGUGCUCAGGGAGGGGUAGUAUCUCUGGUAUGGAGGGC
UUGUCGUGCCCUCCUAGGGCAGCUCUCCAGCCUCUGACCCCCACCUGACA
CCCAGCUCUCACUUGUGGCUCCCAGUAGCUGCUAGCAUGUGGCAGCGGCC
ACACCCCGGGCAACGGCUUCGACAGGCCGGCUAAACCUUGUGAGGGUAGC
CAUCGGGUCAUCGACCCCUGGUGAACCAGGGCUUUGCUCACCCAGCAUGU
GAAGACUGCUUCGGCUGAACAGACGGAAGAAACCAAUAAGAAGGUUCAAC
GGCUGAGAGGGCGACGCAGCAAAGCACUGUGGAGUGCUUAGGGCGUGUUG
GAGCACAAAGGACAACACGGCCAUCCAAUGCAGCUGAGGAAGUCUCCAGA
UGUAACAAUUUUUCGUGCCACUGGACCCAGGCUUCCAACGCCGAGAGAGU
GGGACUGUCUCUGUGCAUCGGCUUUUCCACUUAAAUCUCUUUCACGCACA
AGUAUCUUUGUGCACACUCAUCUAUCCUAACCCCGUCCACCCUCUUCAAG
ACCUGCGGCGAUGGGGGAGUGGCGACGCAACAGGUGGAGGUGACCACUGG
CAGUUGUAGUCACGAUCCUGCACGUAGGCGGCCCACGGACCAGUGGUCGC
UCGGCCCUGUGGGCAGCAGGGACGUUCGGCAGCAUCCUGGGCGACUGAGC
AGCCCUCUCUAGGUUUACGGAUCCGAAUUCUCGACGGAUCGAUCCGUUCU
UUCCGCCUCAGAAGCCAUAGAGCCCACCGCAUCCCCAGCAUGCCUGCUAU
UGUCUUCCCAAUCCUCCCCCUUGCUGUCCUGCCCCACCCCACCCCCCAGA
AUAGAAUGACACCUACUCAGACAAUGCGAUGCAAUUUCCUCAUUUUAUUA
GGAAAGGACAGUGGGAGUGGCACCUUCCAGGGUCAAGGAAGGCACGGGGG
AGGGGCAAACAACAGAUGGCUGGCAACUAGAAGGCACAGUCGAGGCUGAU
CAGCGAAUCCCCCGGCCGCUUUAUCUGGGGGGCAGGGCCUGCAUGUGCAG
GGCGUCGUAGGUGUCCUUGGUGGCGGUGCUCAGGCCCUGGUACAGGCCAU
CGUGGCCCUUGCCGCGCCUCCGCUCGCCCUUCAUGCCGAUCUCGCUGUAG
GCCUCGGCCAUCUUGUCUUUCUGCAGUUCGUUAUACAGGCCUUCCUGGGG
GUUCUUCCGUCUGGGCUUUCCGCCCAUCUCGGGGUCCCGGCCUCUCCGCU
UGUCCAGCACGUCGUACUCUUCCCGUCUGCCCAGGUUCAGCUCGUUGUAC
AGCUGGUUCUGUCCCUGCUGGUAGGCAGGGGCGUCGGCGGAUCUGCUGAA
CUUCACUCUCAGUUCACAUCCUCCUUCUUCUUCUUCUGGAAAUCGGCAGC
UACAGCCAUCUUCCUCUUGAGUAGUUUGUACUGGUCUCAUAAAUGGUUGU
UUGAAUAUAUACAGGAGUUUCUUUCUGCCCCGUUUGUUCCGGUGGUUGCA
GUACAGGGUGAUCACCAGGCUCAGCAGCAGCACGCCACAGGUGCCGGCCA
GAGGGGCCCAGAUGUAGAUGUCGCAGGCGAAGUCCAGGCCUCUGGUGUGC
ACGGCUCCGCCGGCAGCAGGUCUACAGGCUUCGGGCCGCAGAGACAGAGG
CUGGCUGGCGAUUGUUGGGGCUGGGGUGGGAGGUCUAGGGGCAGGGGUGG
UGGUAGGUUUGGCGGGCAGGAACACGGGCACGAAUGAGCUGACGGUGACC
AUUGUACCCUGACCCCAUACGUCUAGUAUGGUGUCUCCACGAUCUCUGGC
GCAGUAGUACACCGCCGUGUCUGCGGCGGUCACAGAACUCAGCUUCAGGG
AGAACUGGUUCUUGGACGUGUCUACGGAUAUGGUGACUCGACUCUUGAGG
GACGGGUUGUAGUAGGUGCUCCCACUAUAGGAGAUACUCCCAAUCCACUC
CAGCCCCUUCCCUGGGGGCUGGCGGAUCCAGCCCCAGUAGUAACUACUAC
UGCUGAUGGAGCCACCAGAGACAGUACAGGUGAGGGACAGGGUCUCCGAA
GGCUUCACCAGUCCUGGGCCCGACUCCUGCAGCUGCAGCUGUCCCUUUGU
GCUGCCCUCGCCAGAGCCAGGCUUGCCGGAGCCGCUGGUGCUGCCUUUGA
UCUCAACCUUGGUCCCUCCGCCAAAAGUGAGGAGGUCGUAUUUUUGCUGA
CAGUAGUAAGUUGCAAAAUCUUCAGGUUGCAGACUGCUGAUGGUGAGAGU
GAAAUCUGUCCCAGAUCCACUGCCACUGAACCUUGAUGGGACCCCACUUU
GCAAACUGGAUGCAGCAUAGAUCAGGAGCUUAGGGGCUUUCCCUGGUUUC
UGCUGAUACCAAUUUAAAUAGCUGCUAAUGCUCUGACUUGCCCGGCAAGU
GAUGGUGACUCUGUCUCCUACAGAUGCAGACAGGGAGGAUGGAGACUGGG
UCAUCUGGAUGUCCGGGCGAGCGGCAUGCAAGAGCAGGGCAAGAGGCAGA
AGCAGGGCGGUCACCGGCAGAGCCAUGGUGGCUUUACCAACAGUACCGGA
AUGCCAAGCUUGGGUCUGGGCUCUUUUAUUGAGCUCGGGGAGCAGAAGCG
CGCGAACAGAAGCGAGAAGCGAACUGAUUGGUUAGUUCAAAUAAGGCACA
GGGUCAUUUCAGGUCCUUGGGGCACCCUGGAAACAUCUGAUGGUUCUCUA
GAAACUGCUGAGGGCGGGACCGCAUCUGGGGACCAUCUGUUCUUGGCCCU
GAGCCGGGGCAGGAACUGCUUACCACAGAUAUCCUGUUUGGCCCAUAUUC
UGCUGUUCCAACUGUUCUUGGCCCUGAGCCGGGGCAGGAACUGCUUACCA
CAGAUAUCCUGUUUGGCCCAUAUUCUGCUGUCUCUCUGUUCCUAACCUUG
AUCCUAGCUUGCCAAACCUACAGGUGGGGUCUUUCAUUCCCCCCUUUUUC
UGGAGACUAAAUAAAUGAAACUGCACAAAGACAAUAGUCAUUCUCGAUCA
CCGAGAGACUACCACUAAAAAAAAAAAAAAAAAAAAAAAAAAAAAAAAAA
AAAAAAAAAAAAAAAAAAAAAAAAAAAAAAAAAAAAAAAAAAAAAA

The BCMA-CAR fusion polypeptide comprised an amino acid sequence of:

(SEQ ID NO: 18209)
DIQMTQSPSSLSASVGDRVTITCRASQSISSYLNWYQQKPGKAPKLLIYA
ASSLQSGVPSRFSGSGSGTDFTLTISSLQPEDFATYYCQQKYDLLTFGGG
TKVEIKGSTSGSGKPGSGEGSTKGQLQLQESGPGLVKPSETLSLTCTVSG
GSISSSSYYWGWIRQPPGKGLEWIGSISYSGSTYYNPSLKSRVTISVDTS
KNQFSLKLSSVTAADTAVYYCARDRGDTILDVWGQGTMVTVSSFVPVFLP
AKPTTTPAPRPPTPAPTIASQPLSLRPEACRPAAGGAVHTRGLDFACDIY
IWAPLAGTCGVLLLSLVITLYCNHRNKRGRKKLLYIFKQPFMRPVQTTQE
EDGCSCRFPEEEEGGCELRVKFSRSADAPAYQQGQNQLYNELNLGRREEY
DVLDKRRGRDPEMGGKPRRKNPQEGLYNELQKDKMAEAYSEIGMKGERRR
GKGHDGLYQGLSTATKDTYDALHMQALPPR

The BCMA-CAR fusion polypeptide comprised a CD8 hinge having an amino acid sequence of:

(SEQ ID NO: 18210)
FVPVFLPAKPTTTPAPRPPTPAPTIASQPLSLRPEACRPAAGGAVHTRGL
DFACD

The BCMA-CAR fusion polypeptide comprised a CD8 transmembrane domain having an amino acid sequence of:

(SEQ ID NO: 18211)
IYIWAPLAGTCGVLLLSLVITLYCNHRN

The BCMA-CAR fusion polypeptide comprised a 4-1BB signaling domain having an amino acid sequence of:

(SEQ ID NO: 18212)
KRGRKKLLYIFKQPFMRPVQTTQEEDGCSCRFPEEEEGGCEL

The BCMA-CAR fusion polypeptide comprised a CD3z signaling domain having an amino acid sequence of:

(SEQ ID NO: 1104)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

The BCMA-CAR fusion polypeptide comprised a GSG Linker having an amino acid sequence of:
GSG

The BCMA-CAR fusion polypeptide comprised a T2A peptide having an amino acid sequence of:

(SEQ ID NO: 18213)
EGRGSLLTCGDVEENPGP

The GFP template RNA comprised a nucleic acid sequence encoding a GFP protein having a protein sequence of:

(SEQ ID NO: 18214)
MVSKGEELFTGVVPILVELDGDVNGHKFSVSGEGEGDATYGKLTLKFICT
TGKLPVPWPTLVTTLTYGVQCFSRYPDHMKQHDFFKSAMPEGYVQERTIF
FKDDGNYKTRAEVKFEGDTLVNRIELKGIDFKEDGNILGHKLEYNYNSHN
VYIMADKQKNGIKVNFKIRHNIEDGSVQLADHYQQNTPIGDGPVLLPDNH
YLSTQSALSKDPNEKRDHMVLLEFVTAAGITLGMDELYK

Cryopreserved primary T cells from a single human donor (HemaCare, donor D328798) were thawed and transferred into PBS with 2% BSA by volume. Cells were counted on a Cellometer K2 Cell Counter (Nexcelom), then pelleted by centrifugation at 400×g for 5 minutes and resuspended in complete T cell media at a density of 1×10⁶ cells/mL. The complete T cell media was comprised of TexMACS basal media (Miltenyi #130-097-196) with 5% human serum AB (Gemini Bio #100-512) and the following cytokines: human IL-2 at 20 ng/mL (Miltenyi Biotec #130-097-746), human IL-7 at 10 ng/ml (Miltenyi Biotec #130-095-362), and human IL-15 at 5 ng/ml (Miltenyi Biotec #130-095-764). TransAct Research Grade T cell activation reagent (Miltenyi Biotec #130-111-160) was added to cell suspension at a volume of 10 ul per 1×10⁶ cells. Activated cells were transferred to flasks and incubated at 37° C., 5% CO₂ for 48 hours. After washing in PBS, T cells were plated in 96-well flat bottom plates at a density of 2.5×10⁵ cells (100 μL) per well in Xvivo15 medium (Lonza) supplemented with ApoE3 10 mg/ml. LNPs comprising the first heterologous gene modifying system and LNPs comprising the second retrotransposon gene modifying system (comprising mRNA encoding RTE-1 and a template RNA encoding GFP or the CAR) were co-administered to the plated human T cells. The LNPs containing the first heterologous gene modifying system were formulated at a mass ratio of 1:0.5:1.25 of gene modifying polypeptide mRNA:RNACS7755:RNACS5630. LNPs containing the first heterologous gene modifying system were used at a dose of 8 μg of LNP per 1×10⁶ cells. The LNPs containing the second retrotransposon gene modifying system were co-mixed such that there was a mass ratio of 1:4 of gene modifying polypeptide mRNA:template for the heterologous gene modifying system comprising GFP template and there was a mass ratio of 1:4-8 of gene modifying polypeptide mRNA:template for the heterologous gene modifying system comprising the CAR template. LNP containing the second retrotransposon gene modifying system were used at a total dose (template and polypeptide) of 2.5-1.25 μg of LNP per 1×10⁶ cells for GFP and 5 μg of LNP per 1×10⁶ cells for CAR.

Transfected cells were returned to incubation at 37° C., 5% CO₂ for four hours, washed in PBS and then cultured for additional 4 days in TexMACS basal media supplemented with 5% human serum AB and the following cytokines: human IL-2 at 20 ng/mL, human IL-7 at 10 ng/ml, and human IL-15 at 5 ng/mL. To determine whether insertion by the second retrotransposon gene modifying system was successful, flow cytometry was used to determine the fraction of cells expressing GFP or BCMA CAR as detected by staining cells with fluorescently labeled Recombinant Human BCMA Protein. To determine whether editing of the TRAC locus by the first heterologous gene modifying system was successful, CD3 levels were measured by flow cytometry as a surrogate readout. Functional loss of TRAC is known to be associated with loss of surface expression of the CD3 complex. To determine whether editing of the B2M locus by the first heterologous gene modifying system was successful, expression of B2M was measured by flow cytometry.

### Results

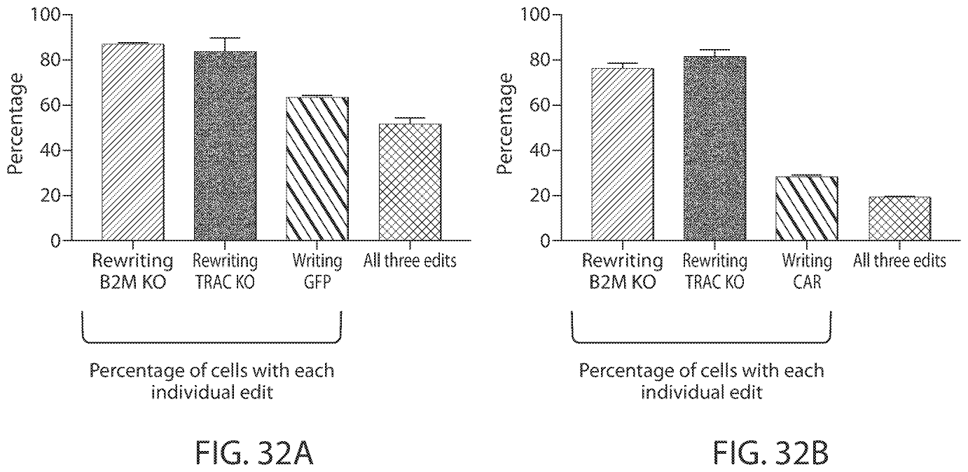

FIG. 32A shows the levels of editing by the first heterologous gene modifying system and the second retrotransposon gene modifying system in the bulk T cell population when the cells were co-transfected with LNPs formulated with a payload comprising the first heterologous gene modifying system and LNPs formulated with a payload comprising the second retrotransposon gene modifying system designed to integrate a GFP sequence into the genome of cells. The results showed that an average of 51.9% of the bulk T cells were edited such that they lacked B2M and TRAC while also expressing GFP. Overall, 87.2% of cells were edited such that they lacked B2M; 84% were edited such that they lacked TRAC; and 63.5% of cells were edited that they expressed GFP.

FIG. 32B shows the levels of editing by the first heterologous gene modifying system and the second retrotransposon gene modifying system in the bulk T cell population when the cells were co-transfected with LNPs formulated with a payload comprising the first heterologous gene modifying system and LNPs formulated a payload comprising the second retrotransposon gene modifying system designed to integrate a CAR sequence into the genome of cells. The results showed that an average of 19.5% of the bulk T cells were edited such that they lacked B2M and TRAC while also expressing CAR. Overall, 76.5% of cells were edited such that they lacked B2M; 81% were edited such that they lacked TRAC; and 28.8% of cells were edited that they expressed CAR.

The results demonstrate that exemplary heterologous gene modifying systems and retrotransposon gene modifying systems can be delivered in RNA format (e.g., by LNP) in combination to achieve multiplex editing of T cells.

### Example 13: Evaluating Translocation Post Multiplexed Gene Editing

This example compares the use exemplary heterologous gene modifying systems containing (i) a heterologous gene modifying polypeptide comprising either (a) a wild type (WT) Cas9-RT fusion protein or (b) an exemplary gene modifying polypeptide; and (ii) and 2 template RNAs to produce an insertion of 5 nucleotides in the human TRAC gene and an insertion of 5 nucleotides in the Beta 2 Microglobulin (B2M) gene in activated T cells, as well as evaluation of said gene modifying activities for translocation events.

Table E11 shows the reagents and components used in two gene modifying systems and control (mock) groups. Sequences of the reagents listed in this table are provided in Tables E12 and E13.

TABLE E11
Experimental conditions

| Gene Modifying system | Reagents used | Description |
|---|---|---|
| Gene Modifying System 1 | RNAIVT1573, | WTCas9-RT + |
| | RNACS6527, | TRAC sgRNA + |
| | RNACS6674 | B2M sgRNA |
| Gene Modifying System 2 | RNAIVT 338, | nExemplary Gene |
| | RNACS5630, | Modifying Polypeptide + |
| | RNACS7755 | TRAC tgRNA + |
| | | B2M tgRNA |
| Mock | Water only | |

In this example, the template RNA of the heterologous gene modifying systems contained:
(1) a gRNA spacer;
(2) a gRNA scaffold;
(3) a heterologous object sequence; and
(4) a primer binding site (PBS) sequence.

The exemplary template RNAs generated and used in the gene modifying systems and the corresponding dosages used in the experiments are given in Table E12.

TABLE E12
Template and single guide RNA: Names, sequences and doses

| Description | Name | Sequence | Dosage |
|---|---|---|---|
| TRAC4_P9R18_ | RNACS5630 | GAGAAUCAAAAUCGGUGAAUGUUUUAGAGCU | 1.5 μM |
| ins5-nick | | AGAAAUAGCAAGUUAAAAUAAGGCUAGUCCG | |
| | | UUAUCAACUUGAAAAAGUGGCACCGAGUCGGU | |
| | | GCCUGUCUGCCUAUUUAGUGCACCGAUUU | |
| | | (SEQ ID NO: 15006) | |
| B2m1_P11R18_ | RNACS7755 | ACUCACGCUGGAUAGCCUCCGUUUUAGAGCUA | 1.5 μM |
| ins5_nick_ | | GAAAUAGCAAGUUAAAAUAAGGCUAGUCCGU | |
| v1 | | UAUCAACUUGAAAAAGUGGCACCGAGUCGGUG | |
| | | CUUUCUGGCCUGGAUGAUGGGCUAUCCAGC | |
| | | (SEQ ID NO: 15007) | |
| TRAC4_sgRNA | RNACS6527 | GAGAAUCAAAAUCGGUGAAUGUUUUAGAGCU | 1.5 μM |
| | | AGAAAUAGCAAGUUAAAAUAAGGCUAGUCCG | |
| | | UUAUCAACUUGAAAAAGUGGCACCGAGUCGGU | |
| | | GCUUUU (SEQ ID NO: 37671) | |
| B2M1_sgRNA | RNACS6674 | ACUCACGCUGGAUAGCCUCCGUUUUAGAGCUA | 1.5 μM |
| | | GAAAUAGCAAGUUAAAAUAAGGCUAGUCCGU | |
| | | UAUCAACUUGAAAAAGUGGCACCGAGUCGGUG | |
| | | CUUUU (SEQ ID NO: 37672) | |

In this example, the exemplary heterologous gene modifying polypeptides of the heterologous gene modifying system contained:
(1) an endonuclease and/or DNA binding domain;
(2) a peptide linker; and
(3) a reverse transcriptase (RT) domain,
   where (1), (2), and (3) are heterologous to each other. Accordingly, the heterologous gene modifying polypeptide in this Example was an example of a heterologous gene modifying polypeptide. A fusion polypeptide comprising a wildtype Cas9 domain, a linker, and an exemplary reverse transcriptase (RT) domain was used as a double strand break-inducing comparator to the exemplary heterologous gene modifying polypeptide.

The exemplary gene heterologous modifying polypeptide and fusion polypeptide of the gene modifying systems comprised the amino acid sequence indicated in Table E13.

TABLE E13
Exemplary Fusion and Gene Modifying Polypeptide Information: Names,
sequence and doses

| Polypeptide | | |
|---|---|---|
| Name/ | | |
| Description | Sequence | Dosage |
| RNAIVT1573/ | MPAAKRVKLDGGDKKYSIGLDIGTNSVGWAVIT | 2 μg |
| WT Cas9-RT | DEYKVPSKKFKVLGNTDRHSIKKNLIGALLFDSGE | |
| | TAEATRLKRTARRRYTRRKNRICYLQEIFSNEMA | |
| | KVDDSFFHRLEESFLVEEDKKHERHPIFGNIVDEV | |
| | AYHEKYPTIYHLRKKLVDSTDKADLRLIYLALAH | |
| | MIKFRGHFLIEGDLNPDNSDVDKLFIQLVQTYNQL | |
| | FEENPINASGVDAKAILSARLSKSRRLENLIAQLPG | |
| | EKKNGLFGNLIALSLGLTPNFKSNFDLAEDAKLQL | |
| | SKDTYDDDLDNLLAQIGDQYADLFLAAKNLSDAI | |
| | LLSDILRVNTEITKAPLSASMIKRYDEHHQDLTLL | |
| | KALVRQQLPEKYKEIFFDQSKNGYAGYIDGGASQ | |
| | EEFYKFIKPILEKMDGTEELLVKLNREDLLRKQRT | |
| | FDNGSIPHQIHLGELHAILRRQEDFYPFLKDNREKI | |
| | EKILTFRIPYYVGPLARGNSRFAWMTRKSEETITP | |
| | WNFEEVVDKGASAQSFIERMTNFDKNLPNEKVLP | |
| | KHSLLYEYFTVYNELTKVKYVTEGMRKPAFLSGE | |
| | QKKAIVDLLFKTNRKVTVKQLKEDYFKKIECFDS | |
| | VEISGVEDRFNASLGTYHDLLKIIKDKDFLDNEEN | |
| | EDILEDIVLTLTLFEDREMIEERLKTYAHLFDDKV | |
| | MKQLKRRRYTGWGRLSRKLINGIRDKQSGKTILD | |
| | FLKSDGFANRNFMQLIHDDSLTFKEDIQKAQVSG | |
| | QGDSLHEHIANLAGSPAIKKGILQTVKVVDELVK | |
| | VMGRHKPENIVIEMARENQTTQKGQKNSRERMK | |
| | RIEEGIKELGSQILKEHPVENTQLQNEKLYLYYLQ | |
| | NGRDMYVDQELDINRLSDYDVDHIVPQSFLKDDS | |
| | IDNKVLTRSDKNRGKSDNVPSEEVVKKMKNYWR | |
| | QLLNAKLITQRKFDNLTKAERGGLSELDKAGFIKR | |
| | QLVETRQITKHVAQILDSRMNTKYDENDKLIREV | |
| | KVITLKSKLVSDFRKDFQFYKVREINNYHHAHDA | |
| | YLNAVVGTALIKKYPKLESEFVYGDYKVYDVRK | |
| | MIAKSEQEIGKATAKYFFYSNIMNFFKTEITLANG | |
| | EIRKRPLIETNGETGEIVWDKGRDFATVRKVLSMP | |
| | QVNIVKKTEVQTGGFSKESILPKRNSDKLIARKKD | |
| | WDPKKYGGFDSPTVAYSVLVVAKVEKGKSKKLK | |
| | SVKELLGITIMERSSFEKNPIDFLEAKGYKEVKKD | |
| | LIIKLPKYSLFELENGRKRMLASAGELQKGNELAL | |
| | PSKYVNFLYLASHYEKLKGSPEDNEQKQLFVEQH | |
| | KHYLDEIIEQISEFSKRVILADANLDKVLSAYNKH | |
| | RDKPIREQAENIIHLFTLTNLGAPAAFKYFDTTIDR | |
| | KRYTSTKEVLDATLIHQSITGLYETRIDLSQLGGD | |
| | GGAEAAAKEAAAKEAAAKEAAAKALEAEAAAK | |
| | EAAAKEAAAKEAAAKAGGTAPLEEEYRLFLEAPI | |
| | QNVTLLEQWKREIPKVWAEINPPGLASTQAPIHV | |
| | QLLSTALPVRVRQYPITLEAKRSLRETIRKFRAAGI | |
| | LRPVHSPWNTPLLPVRKSGTSEYRMVQDLREVNK | |
| | RVETIHPTVPNPYTLLSLLPPDRIWYSVLDLKDAFF | |
| | CIPLAPESQLIFAFEWADAEEGESGQLTWTRLPQG | |
| | FKNSPTLFNEALNRDLQGFRLDHPSVSLLQYVDD | |
| | LLIAADTQAACLSATRDLLMTLAELGYRVSGKKA | |
| | QLCQEEVTYLGFKIHKGSRSLSNSRTQAILQIPVPK | |
| | TKRQVREFLGKIGYCRLFIPGFAELAQPLYAATRP | |
| | GNDPLVWGEKEEEAFQSLKLALTQPPALALPSLD | |
| | KPFQLFVEETSGAAKGVLTQALGPWKRPVAYLSK | |
| | RLDPVAAGWPRCLRAIAAAALLTREASKLTFGQD | |
| | IEITSSHNLESLLRSPPDKWLTNARITQYQVLLLDP | |
| | PRVRFKQTAALNPATLLPETDDTLPIHHCLDTLDS | |
| | LTSTRPDLTDQPLAQAEATLFTDGSSYIRDGKRYA | |
| | GAAVVTLDSVIWAEPLPIGTSAQKAELIALTKALE | |
| | WSKDKSVNIYTDSRYAFATLHVHGMIYRERGWL | |
| | TAGGKAIKNAPEILALLTAVWLPKRVAVMHCKG | |
| | HQKDDAPTSTGNRRADEVAREVAIRPLSTQATISA | |
| | GKRTADGSEFEKRTADGSEFESPKKKAKVE (SEQ | |
| | ID NO: 18215) | |
| RNAIVT 338 / | MPAAKRVKLDGGDKKYSIGLDIGTNSVGWAVIT | 2 μg |
| Exemplary | DEYKVPSKKFKVLGNTDRHSIKKNLIGALLFDSGE | |
| gene | TAEATRLKRTARRRYTRRKNRICYLQEIFSNEMA | |
| modifying | KVDDSFFHRLEESFLVEEDKKHERHPIFGNIVDEV | |
| polypeptide | AYHEKYPTIYHLRKKLVDSTDKADLRLIYLALAH | |
| | MIKFRGHFLIEGDLNPDNSDVDKLFIQLVQTYNQL | |
| | FEENPINASGVDAKAILSARLSKSRRLENLIAQLPG | |
| | EKKNGLFGNLIALSLGLTPNFKSNFDLAEDAKLQL | |
| | SKDTYDDDLDNLLAQIGDQYADLFLAAKNLSDAI | |
| | LLSDILRVNTEITKAPLSASMIKRYDEHHQDLTLL | |
| | KALVRQQLPEKYKEIFFDQSKNGYAGYIDGGASQ | |
| | EEFYKFIKPILEKMDGTEELLVKLNREDLLRKQRT | |
| | FDNGSIPHQIHLGELHAILRRQEDFYPFLKDNREKI | |
| | EKILTFRIPYYVGPLARGNSRFAWMTRKSEETITP | |
| | WNFEEVVDKGASAQSFIERMTNFDKNLPNEKVLP | |
| | KHSLLYEYFTVYNELTKVKYVTEGMRKPAFLSGE | |
| | QKKAIVDLLFKTNRKVTVKQLKEDYFKKIECFDS | |
| | VEISGVEDRFNASLGTYHDLLKIIKDKDFLDNEEN | |
| | EDILEDIVLTLTLFEDREMIEERLKTYAHLFDDKV | |
| | MKQLKRRRYTGWGRLSRKLINGIRDKQSGKTILD | |
| | FLKSDGFANRNFMQLIHDDSLTFKEDIQKAQVSG | |
| | QGDSLHEHIANLAGSPAIKKGILQTVKVVDELVK | |
| | VMGRHKPENIVIEMARENQTTQKGQKNSRERMK | |
| | RIEEGIKELGSQILKEHPVENTQLQNEKLYLYYLQ | |
| | NGRDMYVDQELDINRLSDYDVDHIVPQSFLKDDS | |
| | IDNKVLTRSDKARGKSDNVPSEEVVKKMKNYWR | |
| | QLLNAKLITQRKFDNLTKAERGGLSELDKAGFIKR | |
| | QLVETRQITKHVAQILDSRMNTKYDENDKLIREV | |
| | KVITLKSKLVSDFRKDFQFYKVREINNYHHAHDA | |
| | YLNAVVGTALIKKYPKLESEFVYGDYKVYDVRK | |
| | MIAKSEQEIGKATAKYFFYSNIMNFFKTEITLANG | |
| | EIRKRPLIETNGETGEIVWDKGRDFATVRKVLSMP | |
| | QVNIVKKTEVQTGGFSKESILPKRNSDKLIARKKD | |
| | WDPKKYGGFDSPTVAYSVLVVAKVEKGKSKKLK | |
| | SVKELLGITIMERSSFEKNPIDFLEAKGYKEVKKD | |
| | LIIKLPKYSLFELENGRKRMLASAGELQKGNELAL | |
| | PSKYVNFLYLASHYEKLKGSPEDNEQKQLFVEQH | |
| | KHYLDEIIEQISEFSKRVILADANLDKVLSAYNKH | |
| | RDKPIREQAENIIHLFTLTNLGAPAAFKYFDTTIDR | |
| | KRYTSTKEVLDATLIHQSITGLYETRIDLSQLGGD | |
| | GGAEAAAKEAAAKEAAAKEAAAKALEAEAAAK | |
| | EAAAKEAAAKEAAAKAGGTAPLEEEYRLFLEAPI | |
| | QNVTLLEQWKREIPKVWAEINPPGLASTQAPIHV | |
| | QLLSTALPVRVRQYPITLEAKRSLRETIRKFRAAGI | |
| | LRPVHSPWNTPLLPVRKSGTSEYRMVQDLREVNK | |
| | RVETIHPTVPNPYTLLSLLPPDRIWYSVLDLKDAFF | |
| | CIPLAPESQLIFAFEWADAEEGESGQLTWTRLPQG | |
| | FKNSPTLFNEALNRDLQGFRLDHPSVSLLQYVDD | |
| | LLIAADTQAACLSATRDLLMTLAELGYRVSGKKA | |
| | QLCQEEVTYLGFKIHKGSRSLSNSRTQAILQIPVPK | |
| | TKRQVREFLGKIGYCRLFIPGFAELAQPLYAATRP | |
| | GNDPLVWGEKEEEAFQSLKLALTQPPALALPSLD | |
| | KPFQLFVEETSGAAKGVLTQALGPWKRPVAYLSK | |
| | RLDPVAAGWPRCLRAIAAAALLTREASKLTFGQD | |
| | IEITSSHNLESLLRSPPDKWLTNARITQYQVLLLDP | |
| | PRVRFKQTAALNPATLLPETDDTLPIHHCLDTLDS | |
| | LTSTRPDLTDQPLAQAEATLFTDGSSYIRDGKRYA | |
| | GAAVVTLDSVIWAEPLPIGTSAQKAELIALTKALE | |
| | WSKDKSVNIYTDSRYAFATLHVHGMIYRERGWL | |
| | TAGGKAIKNAPEILALLTAVWLPKRVAVMHCKG | |
| | HQKDDAPTSTGNRRADEVAREVAIRPLSTQATISA | |
| | GKRTADGSEFEKRTADGSEFESPKKKAKVE (SEQ | |
| | ID NO: 1372) | |

### T-Cell Culturing, Activation and Nucleofection:

Activated T cells were used for this experiment. Prior to nucleofection, T cells were cultured with TexMACS supplemented with 5% human AB Serum, IL2 at 20 IU/ml, IL7 at 10 ng/ml, IL15 at 5 ng/ml in each well at 37° C., 5% CO₂ for 2 days and stimulated using T Cell TransAct. For the nucleofection of the first heterologous gene modifying system, 2000 ng of the first heterologous gene modifying polypeptide encoding mRNA was combined with TRAC and B2M single-guide RNAs (each sgRNA at a final conc. 1.5 μM) in RNA format. For the nucleofection of the second heterologous gene modifying system, 2000 ng of the second heterologous gene modifying polypeptide encoding mRNA was combined with TRAC and B2M template guide RNAs (each tgRNA at a final conc. 1.5 μM) in RNA format.

The RNA mixture (comprising the first or the second heterologous gene modifying system) was added to 250,000 activated T cells/condition in a total of 20 μL of Lonza P3 buffer. The T cells were then nucleofected in 96-well nucleofection cassettes using the program called Human T cell, stimulated (Program code: EO 115). After nucleofection, the cells were incubated at room temperature for 10 minutes and then transferred to 96-well plates containing 180 μL of TexMACS supplemented with 5% human AB Serum, IL2 at 20 IU/ml, IL7 at 10 ng/ml in each well and cultured at 37° C., 5% CO₂ for either 3 days prior to genomic extraction, or, for 4 days prior to flow cytometry analysis.

### Flow Cytometry:

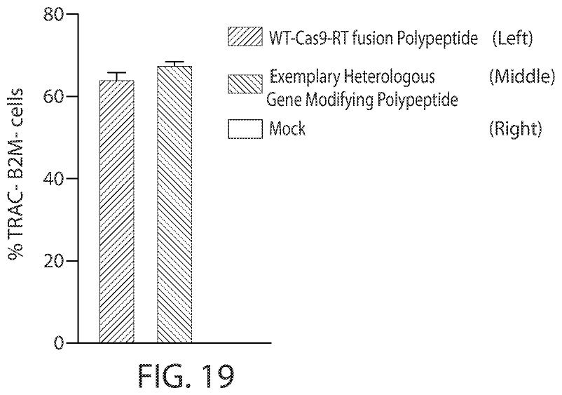

To determine whether editing of the TRAC and B2M loci by the first and second heterologous gene modifying systems was successful, CD3 and B2M levels were measured. Functional loss of TRAC is known to be associated with loss of surface expression of the CD3 complex. Similarly, functional loss of B2M is associated with the loss of surface expression of beta2-microglobulin and can be detected with B2M antibody staining. As shown in FIG. 19, editing at the TRAC and B2M loci by the first and second heterologous gene modifying systems was successful with similar editing efficiencies using gene modifying systems comprising either a WT Cas9-RT fusion polypeptide or an exemplary gene modifying polypeptide, and editing was not observed in mock treated cells.

Translocation Measurements Using Digital PCR (dPCR):

To examine translocations by dPCR, cells were collected on the third day post nucleofection. Genomic DNA was extracted using a DNAdvance kit from Beckman Coulter by lysing the cells overnight in the Lysis LBH buffer with Proteinase K, and 1M DTT. The lysed cells were extracted as per manufacturer instructions using the Biomek i5 liquid handler. Post extraction, the concentrations of extracted genomic DNA were measured using the Quant-it 1× dsDNA assay kit, High Sensitivity from Thermofisher.

Translocation events were quantitated using the QIAcuity Eight digital PCR system from Qiagen. The reaction mix was prepared with the QIAcuity Probe PCR Kit according to manufacturer instructions and loaded onto 96-well 8.5 k nanochamber plates. Reaction partitioning was performed by the instrument's default protocol. Thermocycling and imaging were performed as described below. CNV (translocation occurrence) was calculated as such: [target amplicon]/[reference gene amplicon]*ploidy. Ploidy was assumed to be 2.

| Temperature | Time | Cycles |
|---|---|---|
| 95° C. hot start | 2 min | 1 |
| 94° C. denature | 30 sec | < |
| 58° C. anneal/ext | 1 min | 40X> |

| Assay | Probe (400 nM) | F primer (800 nM) | R primer (800 nM) |
|---|---|---|---|
| Balanced | /56- | GAACTCTCTTA | CCTTTCTTGCA |
| 1 | FAM/TGGTCTTAG/ZEN/GAACTTTG | AGACTACCTTT | GTTGAGTTGTG |
| | CTGCCACA/3IABKFQ/ (SEQ ID NO: | CTCC (SEQ ID | (SEQ ID NO: |
| | 37673) | NO: 37674) | 37675) |
| Balanced | /56- | GAATCTGAAC | ATCTTAAGCTG |
| 2 | FAM/ACGACCCAT/ZEN/ACACCCA | TCTCTTCAACC | CTGGGTATTGT |
| | AAGGATTGAG/3IABKFQ/ (SEQ ID | TTTG (SEQ ID | (SEQ ID NO: |
| | NO: 37676) | NO: 37677) | 37678) |
| Dicentric | /56- | GAGGTGTCCG | AGAATCTGAAC |
| | FAM/TGGTCTTAG/ZEN/GAACTTTG | TGTTCCTAAA | TCTCTTCAACC |
| | CTGCCACA/3IABKFQ/ (SEQ ID NO: | (SEQ ID NO: | T (SEQ ID NO: |
| | 37673) | 37679) | 37680) |
| Acentric | /56- | CCTTTCTTGCA | GACCTCAATCC |
| | FAM/ACTGTAAAC/ZEN/CTACACA | GTTGAGTTGT | TTTGGGTGTAT |
| | GGTGAGTTGGC/3IABKFQ/ (SEQ ID | G (SEQ ID NO: | (SEQ ID NO: |
| | NO: 37681) | 37675) | 37682) |
| RPP30 | /5HEX/TCCCTAGGT/ZEN/GGCCTG | TCCAGGAGGG | ATGGTCCGTCT |
| | AGCAG/3IABKFQ/ (SEQ ID NO: | AGAATTGATG | CAGGAAA (SEQ |
| | 37683) or /56- | (SEQ ID NO: | ID NO: 37686) |
| | ROXN/AGAGAACTGAGTGGAGTCA | 37685) | |
| | AGGCTGA/3IAbRQSp/ (SEQ ID NO: | | |
| | 37684) | | |

| | | Exposure | |
|---|---|---|---|
| Dye | Color | time (ms) | Gain |
| FAM | Green | 600 | 6 |
| HEX | Yellow | 500 | 6 |
| ROX | Red | 300 | 4 |

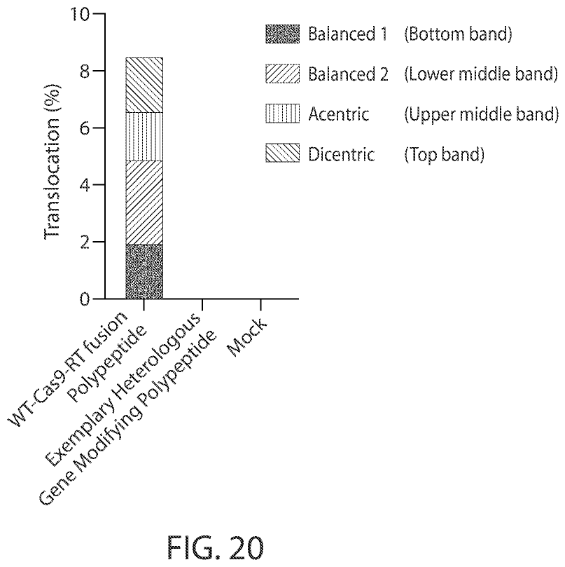

Total translocation % was calculated as a sum of all the 4 different translocation measurements (Balanced 1, Balanced 2, Acentric and Dicentric). FIG. 20 shows a graph of translocation % for cells transfected with gene modifying systems comprising WT Cas9-RT fusion polypeptide or an exemplary gene modifying polypeptide, or cells given a mock treatment. The results show that multiplexed editing with WTCas9-RT fusion polypeptide resulted in close to 8.53% translocationMultiplexed editing with WTCas9-RT yielded close to 8.53% translocation (FIG. 20) while editing with the exemplary heterologous gene modifying system showed background levels of translocation.

Overall, these results demonstrate that multiplexed knock-out of TRAC and B2M using an exemplary heterologous gene modifying peptide does not produce translocations in T cells, whereas a WT-Cas9-RT fusion polypeptide, which induces double strand using similarly targeted sgRNAs does produce translocations.

### Example 14: LNPs can Deliver of RNA Payloads Comprising Gene Modifying Systems to T Cells In Vivo

LNPs were tested for their ability to deliver gene modifying systems to T cells in vivo. The gene modifying system comprised either an mRNA encoding a wild-type Cas9-RT fusion protein and a gRNA specific for the first exon of the human Beta 2 Microglobulin (B2M) gene or a Cas9 nickase fused to an RT and a template RNA suitable to produce an insertion of five nucleotides (TAGTG) into the first exon of B2M. The introduction of the wtCas9-RT and gRNA lead to the introduction and repair of a DNA double-stranded break induced at this site which lead to the formation of small insertions and deletions (indels), largely resulting in frameshifts and thus loss of B2M expression from the cell surface.

Introduction of the nCas9-RT and B2M template RNA lead to a single-stranded DNA break and the insertion of the 5 nucleotides thereby generating a frameshift mutation which resulted in loss of expression of B2M from the cell surface. The LNPs were administered to humanized mice and evaluated for their effectiveness in delivering the payloads in vivo as measured by the percentage of either B2M indels in T cells or the percentage of reads including the desired insertion of 5 nucleotides (TAGTG) in the first exon of B2M (assayed by Ampseq).

As described above, LNPs containing Cas9-RT and gRNA were formulated to encapsulate an mRNA encoding a wild-type Cas9 molecule fused to a reverse transcriptase domain (“wtCas9-RT,” SEQ ID NO: 18215) and the gRNA (“B2M gRNA1,” SEQ ID NO: 18216) at a mass ratio of 1:1. The LNPs containing nCas9-RT and template RNA were formulated to encapsulate an mRNA encoding Cas9 nickase fused to an RT (“nCas9-RT,” SEQ ID NO: 1372) and the template RNA (“B2M template RNA,” SEQ ID NO: 17627) at a mass ratio of 1:1. The gene modifying polypeptides comprising wtCas9-RT and nCas9-RT comprise the amino acid sequences indicated in Table 14A.

TABLE 14A
Gene modifying polypeptide sequences

| | | SEQ ID |
|---|---|---|
| Name | Amino Acid sequence | NO |
| wtCas | MPAAKRVKLDGGDKKYSIGLDIGTNSVGWAVITDEYKVPSKKFKVLGNTDRHSIKK | 18215 |
| 9 RT | NLIGALLFDSGETAEATRLKRTARRRYTRRKNRICYLQEIFSNEMAKVDDSFFHRLEE | |
| | SFLVEEDKKHERHPIFGNIVDEVAYHEKYPTIYHLRKKLVDSTDKADLRLIYLALAHM | |
| | IKFRGHFLIEGDLNPDNSDVDKLFIQLVQTYNQLFEENPINASGVDAKAILSARLSKSR | |
| | RLENLIAQLPGEKKNGLFGNLIALSLGLTPNFKSNFDLAEDAKLQLSKDTYDDDLDNL | |
| | LAQIGDQYADLFLAAKNLSDAILLSDILRVNTEITKAPLSASMIKRYDEHIIQDLTLLK | |
| | ALVRQQLPEKYKEIFFDQSKNGYAGYIDGGASQEEFYKFIKPILEKMDGTEELLVKLN | |
| | REDLLRKQRTFDNGSIPHQIHLGELHAILRRQEDFYPFLKDNREKIEKILTFRIPYYVGP | |
| | LARGNSRFAWMTRKSEETITPWNFEEVVDKGASAQSFIERMTNFDKNLPNEKVLPKH | |
| | SLLYEYFTVYNELTKVKYVTEGMRKPAFLSGEQKKAIVDLLFKTNRKVTVKQLKED | |
| | YFKKIECFDSVEISGVEDRFNASLGTYHDLLKIIKDKDFLDNEENEDILEDIVLTLTLFE | |
| | DREMIEERLKTYAHLFDDKVMKQLKRRRYTGWGRLSRKLINGIRDKQSGKTILDFLK | |
| | SDGFANRNFMQLIHDDSLTFKEDIQKAQVSGQGDSLHEHIANLAGSPAIKKGILQTVK | |
| | VVDELVKVMGRHKPENIVIEMARENQTTQKGQKNSRERMKRIEEGIKELGSQILKEH | |
| | PVENTQLQNEKLYLYYLQNGRDMYVDQELDINRLSDYDVDHIVPQSFLKDDSIDNK | |
| | VLTRSDKNRGKSDNVPSEEVVKKMKNYWRQLLNAKLITQRKFDNLTKAERGGLSEL | |
| | DKAGFIKRQLVETRQITKHVAQILDSRMNTKYDENDKLIREVKVITLKSKLVSDFRKD | |
| | FQFYKVREINNYHHAHDAYLNAVVGTALIKKYPKLESEFVYGDYKVYDVRKMIAKS | |
| | EQEIGKATAKYFFYSNIMNFFKTEITLANGEIRKRPLIETNGETGEIVWDKGRDFATVR | |
| | KVLSMPQVNIVKKTEVQTGGFSKESILPKRNSDKLIARKKDWDPKKYGGFDSPTVAY | |
| | SVLVVAKVEKGKSKKLKSVKELLGITIMERSSFEKNPIDFLEAKGYKEVKKDLIIKLPK | |
| | YSLFELENGRKRMLASAGELQKGNELALPSKYVNFLYLASHYEKLKGSPEDNEQKQ | |
| | LFVEQHKHYLDEIIEQISEFSKRVILADANLDKVLSAYNKHRDKPIREQAENIIHLFTLT | |
| | NLGAPAAFKYFDTTIDRKRYTSTKEVLDATLIHQSITGLYETRIDLSQLGGDGGAEAA | |
| | AKEAAAKEAAAKEAAAKALEAEAAAKEAAAKEAAAKEAAAKAGGTAPLEEEYRLF | |
| | LEAPIQNVTLLEQWKREIPKVWAEINPPGLASTQAPIHVQLLSTALPVRVRQYPITLEA | |
| | KRSLRETIRKFRAAGILRPVHSPWNTPLLPVRKSGTSEYRMVQDLREVNKRVETIHPT | |
| | VPNPYTLLSLLPPDRIWYSVLDLKDAFFCIPLAPESQLIFAFEWADAEEGESGQLTWTR | |
| | LPQGFKNSPTLFNEALNRDLQGFRLDHPSVSLLQYVDDLLIAADTQAACLSATRDLL | |
| | MTLAELGYRVSGKKAQLCQEEVTYLGFKIHKGSRSLSNSRTQAILQIPVPKTKRQVRE | |
| | FLGKIGYCRLFIPGFAELAQPLYAATRPGNDPLVWGEKEEEAFQSLKLALTQPPALAL | |
| | PSLDKPFQLFVEETSGAAKGVLTQALGPWKRPVAYLSKRLDPVAAGWPRCLRAIAA | |
| | AALLTREASKLTFGQDIEITSSHNLESLLRSPPDKWLTNARITQYQVLLLDPPRVRFKQ | |
| | TAALNPATLLPETDDTLPIHHCLDTLDSLTSTRPDLTDQPLAQAEATLFTDGSSYIRDG | |
| | KRYAGAAVVTLDSVIWAEPLPIGTSAQKAELIALTKALEWSKDKSVNIYTDSRYAFA | |
| | TLHVHGMIYRERGWLTAGGKAIKNAPEILALLTAVWLPKRVAVMHCKGHQKDDAP | |
| | TSTGNRRADEVAREVAIRPLSTQATISAGKRTADGSEFEKRTADGSEFESPKKKAKVE | |
| nCas9- | MPAAKRVKLDGGDKKYSIGLDIGTNSVGWAVITDEYKVPSKKFKVLGNTDRHSIKK | 1372 |
| RT | NLIGALLFDSGETAEATRLKRTARRRYTRRKNRICYLQEIFSNEMAKVDDSFFHRLEE | |
| | SFLVEEDKKHERHPIFGNIVDEVAYHEKYPTIYHLRKKLVDSTDKADLRLIYLALAHM | |
| | IKFRGHFLIEGDLNPDNSDVDKLFIQLVQTYNQLFEENPINASGVDAKAILSARLSKSR | |
| | RLENLIAQLPGEKKNGLFGNLIALSLGLTPNFKSNFDLAEDAKLQLSKDTYDDDLDNL | |
| | LAQIGDQYADLFLAAKNLSDAILLSDILRVNTEITKAPLSASMIKRYDEHHQDLTLLK | |
| | ALVRQQLPEKYKEIFFDQSKNGYAGYIDGGASQEEFYKFIKPILEKMDGTEELLVKLN | |
| | REDLLRKQRTFDNGSIPHQIHLGELHAILRRQEDFYPFLKDNREKIEKILTFRIPYYVGP | |
| | LARGNSRFAWMTRKSEETITPWNFEEVVDKGASAQSFIERMTNFDKNLPNEKVLPKH | |
| | SLLYEYFTVYNELTKVKYVTEGMRKPAFLSGEQKKAIVDLLFKTNRKVTVKQLKED | |
| | YFKKIECFDSVEISGVEDRFNASLGTYHDLLKIIKDKDFLDNEENEDILEDIVLTLTLFE | |
| | DREMIEERLKTYAHLFDDKVMKQLKRRRYTGWGRLSRKLINGIRDKQSGKTILDFLK | |
| | SDGFANRNFMQLIHDDSLTFKEDIQKAQVSGQGDSLHEHIANLAGSPAIKKGILQTVK | |
| | VVDELVKVMGRHKPENIVIEMARENQTTQKGQKNSRERMKRIEEGIKELGSQILKEH | |
| | PVENTQLQNEKLYLYYLQNGRDMYVDQELDINRLSDYDVDHIVPQSFLKDDSIDNK | |
| | VLTRSDKARGKSDNVPSEEVVKKMKNYWRQLLNAKLITQRKFDNLTKAERGGLSEL | |
| | DKAGFIKRQLVETRQITKHVAQILDSRMNTKYDENDKLIREVKVITLKSKLVSDFRKD | |
| | FQFYKVREINNYHHAHDAYLNAVVGTALIKKYPKLESEFVYGDYKVYDVRKMIAKS | |
| | EQEIGKATAKYFFYSNIMNFFKTEITLANGEIRKRPLIETNGETGEIVWDKGRDFATVR | |
| | KVLSMPQVNIVKKTEVQTGGFSKESILPKRNSDKLIARKKDWDPKKYGGFDSPTVAY | |
| | SVLVVAKVEKGKSKKLKSVKELLGITIMERSSFEKNPIDFLEAKGYKEVKKDLIIKLPK | |
| | YSLFELENGRKRMLASAGELQKGNELALPSKYVNFLYLASHYEKLKGSPEDNEQKQ | |
| | LFVEQHKHYLDEIIEQISEFSKRVILADANLDKVLSAYNKHRDKPIREQAENIIHLFTLT | |
| | NLGAPAAFKYFDTTIDRKRYTSTKEVLDATLIHQSITGLYETRIDLSQLGGDGGAEAA | |
| | AKEAAAKEAAAKEAAAKALEAEAAAKEAAAKEAAAKEAAAKAGGTAPLEEEYRLF | |
| | LEAPIQNVTLLEQWKREIPKVWAEINPPGLASTQAPIHVQLLSTALPVRVRQYPITLEA | |
| | KRSLRETIRKFRAAGILRPVHSPWNTPLLPVRKSGTSEYRMVQDLREVNKRVETIHPT | |
| | VPNPYTLLSLLPPDRIWYSVLDLKDAFFCIPLAPESQLIFAFEWADAEEGESGQLTWTR | |
| | LPQGFKNSPTLFNEALNRDLQGFRLDHPSVSLLQYVDDLLIAADTQAACLSATRDLL | |
| | MTLAELGYRVSGKKAQLCQEEVTYLGFKIHKGSRSLSNSRTQAILQIPVPKTKRQVRE | |
| | FLGKIGYCRLFIPGFAELAQPLYAATRPGNDPLVWGEKEEEAFQSLKLALTQPPALAL | |
| | PSLDKPFQLFVEETSGAAKGVLTQALGPWKRPVAYLSKRLDPVAAGWPRCLRAIAA | |
| | AALLTREASKLTFGQDIEITSSHNLESLLRSPPDKWLTNARITQYQVLLLDPPRVRFKQ | |
| | TAALNPATLLPETDDTLPIHHCLDTLDSLTSTRPDLTDQPLAQAEATLFTDGSSYIRDG | |
| | KRYAGAAVVTLDSVIWAEPLPIGTSAQKAELIALTKALEWSKDKSVNIYTDSRYAFA | |
| | TLHVHGMIYRERGWLTAGGKAIKNAPEILALLTAVWLPKRVAVMHCKGHQKDDAP | |
| | TSTGNRRADEVAREVAIRPLSTQATISAGKRTADGSEFEKRTADGSEFESPKKKAKVE | |

The B2M gRNA and template RNA sequences are provided in Table 14B.

Nucleotide modifications are noted as follows: phosphorothioate linkages denoted by an asterisk, 2′-O-methyl groups denoted by an ‘m’ preceding a nucleotide.

TABLE 14B
B2M gRNA and template sequences

| | | SEQ ID |
|---|---|---|
| Name | Sequence | NO |
| B2M | mA*mC*mU*rCrArCrGrCrUrGrGrArUrArGrCrCrUrCrCrGrUr | 18216 |
| gRNA1 | UrUrUrArGrAmGmCmUmAmGmAmAmAmUmAmGmCrArA | |
| | rGrUrUrArArArArUrArArGrGrCrUrArGrUrCrCrGrUrUrArUrC | |
| | rAmAmCmUmUmGmAmAmAmAmAmGmUmGmGmCmAm | |
| | CmCmGmAmGmUmCmGmGmUmGmCmU*mU*mU*mU | |
| B2M | mA*mC*mU*rCrArCrGrCrUrGrGrArUrArGrCrCrUrCrCrGrUr | 17627 |
| template | UrUrUrArGrAmGmCmUmAmGmAmAmAmUmAmGmCrArA | |
| RNA (with | rGrUrUrArArArArUrArArGrGrCrUrArGrUrCrCrGrUrUrArUrC | |
| modifications) | rAmAmCmUmUmGmAmAmAmAmAmGmUmGmGmCmAm | |
| | CmCmGmAmGmUmCmGmGmUmGmCrUrUrUrCrUrGrGrCr | |
| | CrUrGrGrArUrGrArUrGrGrGrCrUrArUrCrC*mA*mG*mC | |
| B2M | ACUCACGCUGGAUAGCCUCCGUUUUAGAGCUAGAAAU | 17963 |
| template | AGCAAGUUAAAAUAAGGCUAGUCCGUUAUCAACUUG | |
| RNA | AAAAAGUGGCACCGAGUCGGUGCUUUCUGGCCUGGA | |
| (without | UGAUGGGCUAUCCAGC | |
| modifications) | | |

The B2M gRNA1 contained a spacer and a gRNA scaffold as provided in Table 14C.

TABLE 14C
B2M spacer and gRNA scaffold sequences

| | | SEQ |
|---|---|---|
| | | ID |
| Name | Sequence | NO |
| gRNA | ACUCACGCUGGAUAGCCUCC | 18195 |
| spacer | | |
| gRNA | GUUUUAGAGCUAGAAAUAGCAAGUUAAAAUA | 18217 |
| scaffold | AGGCUAGUCCGUUAUCAACUUGAAAAAGUGG | |
| | CACCGAGUCGGUGCUUUU | |

The B2M template RNA contained:
1. a gRNA spacer;
2. a gRNA scaffold;
3. a heterologous object sequence; and
4. a primer binding site (PBS) sequence.

The heterologous object sequences and PBS sequences were designed to introduce an insertion of 5 nucleotides (TAGTG) in the first exon of B2M, which generates a frameshift mutation.

### Preparation and Dosing of a Humanized Mouse Model

Briefly, female NSG mice between 6-8 weeks of age (Catalog #005557) were purchased from Jackson Labs (Bar Harbor, ME). Following acclimation to the vivarium for 3-5 days, animals received a tail vein injection of 5×10⁶ human CD3+ T cells and waited for 2-3 weeks for T cell engraftment. For each in vivo study, “humanized” NSG mice were randomized into groups of 4 animals and baseline weights were obtained. A group of four animals was then treated via intravenous tail vein injection with 100 μL of either the LNPs comprising wtCas9-RT mRNA and B2M gRNA or the LNPs comprising nCas9-RT and the B2M template RNA at a dose of 4 mg/kg. Following a 16-hour incubation, animals were euthanized via CO2 asphyxiation, weighed and their spleens were collected and the splenic cells were isolated.

### Flow Cytometry

Splenic cells (1×10⁶ in 0.1 ml PBS) from each test mouse were subsequently aliquoted to individual wells in 96 well plates and antibody fluorochrome conjugate mixtures (Table 14D) were added to the cells in a total of 0.1 ml. Following a 30-minute incubation at room temperature, the plates were centrifuged for 5 minutes at 500G and supernatants were removed. The cells were then resuspended in 0.2 mL PBS and evaluated on an Aligent NovoCyte Quanteon (2010098) or Cytek Northern Lights (N7-00008) flow cytometer using FlowJo software (Ashland, OR). Cells stained with relevant isotype control fluorochrome conjugated antibodies were used to set the negative control gates. Total T cells were defined as CD45+CD3+CD5+. CD4+ and CD8+ T cells were defined as CD45+CD3+CD5+CD4+ and CD45+CD3+CD5+CD8+, respectively.

TABLE 14D
Flow cytometry antibodies

| Marker | Fluorophore | Clone | Vendor | Catalog |
|---|---|---|---|---|
| Live/Dead | Fixable | | ThermoFisher | L34959 |
| | Yellow | | | |
| hCD45 | PE-Cy7 | HI30 | Biolegends | 304016 |
| CD3 | BV421 | OKT3 | Biolegends | 317344 |
| CD4 | BV605 | OKT4 | Biolegends | 317438 |
| CD5 | BV510 | L17F12 | Biolegends | 364018 |
| GFP | | | | |
| CD8 | PE | SK1 | Biolegends | 344706 |
| CCR7 | APC | G043H7 | Biolegends | 353214 |
| mCD45 | PE Cy5 | 30-F11 | Biolegends | 103110 |
| CD45RA | BV785 | HI100 | Biolegends | 304140 |

### Results

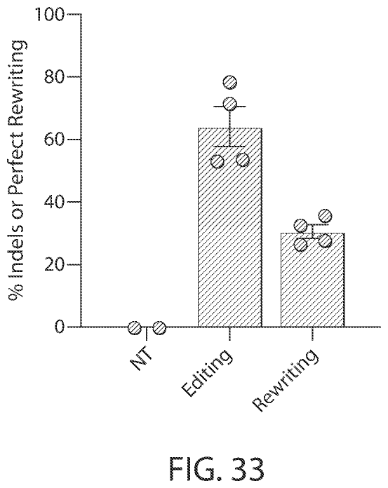

FIG. 33 shows that the administered LNPs can deliver gene modifying systems to human T cells in an animal model effectively, leading to in vivo gene editing in T cells.

FIG. 33 shows that LNPs formulated with the wtCas9-RT mRNA and B2M gRNA payload (“Editing” in FIG. 33) resulted in a mean of about 64% indels in B2M in T cells, as measured by Ampseq. Control animals that did not receive LNPs did not exhibit any B2M indels. FIG. 33 shows that the LNPs formulated with the nCas9-RT and B2M template RNA payload (“Rewriting” in FIG. 33) resulted in the elimination of B2M from the surface of a mean of about 31% of the human T cells, as measured by Ampseq, showing that rewriting led to B2M knockout in these cells in vivo. The control animals that did not receive LNPs did not exhibit any reduction in B2M.

### Example 15: Heterologous Gene Modifying Polypeptide Selection by Pooled Screening in HEK293T & U20S Cells

This example describes the use of an RNA heterologous gene modifying system for the targeted editing of a coding sequence in the human genome. More specifically, this example describes the infection of HEK293T and U2OS cells with a library of gene modifying candidates, followed by transfection of a template guide RNA (tgRNA) for in vitro gene modifying in the cells, e.g., as a means of evaluating a new heterologous gene modifying polypeptide for editing activity in human cells by a pooled screening approach.

The heterologous gene modifying polypeptide library candidates assayed herein each comprise: 1) a S. pyogenes (Spy) Cas9 nickase containing an N863A mutation that inactivates one endonuclease active site; 2) one of the 122 peptide linkers depicted at Table 10; and 3) a reverse transcriptase (RT) domain from Table 6 of retroviral origin. The particular retroviral RT domains utilized were selected if they were expected to function as a monomer. For each selected RT domain, the wild-type sequences were tested, as well as versions with point mutations installed in the primary wild-type sequence. In particular, 143 RT domains were tested, either wild type or containing various mutations. In total, 17,446 Cas-linker-RT heterologous gene modifying polypeptides were tested.

The system described here is a two-component system comprising: 1) an expression plasmid encoding a human codon-optimized heterologous gene modifying polypeptide library candidate within a lentiviral cassette, and 2) a tgRNA expression plasmid expressing a non-coding tgRNA sequence that is recognized by Cas and localizes it to the genomic locus of interest, and that also templates reverse transcription of the desired edit into the genome by the RT domain, driven by a U6 promoter. The lentiviral cassette comprises: (i) a CMV promoter for expression in mammalian cells; (ii) a heterologous gene modifying polypeptide library candidate as shown; (iii) a self-cleaving T2A polypeptide; (iv) a puromycin resistance gene enabling selection in mammalian cells; and (v) a polyA tail termination signal.

To prepare a pool of cells expressing heterologous gene modifying polypeptide library candidates, HEK293T or U2OS cells were transduced with pooled lentiviral preparations of the gene modifying candidate plasmid library. HEK293 Lenti-X cells were seeded in 15 cm plates (12×10⁶ cells) prior to lentiviral plasmid transfection. Lentiviral plasmid transfection using the Lentiviral Packaging Mix (Biosettia, 27 μg) and the plasmid DNA for the gene modifying candidate library (27 μg) was performed the following day using Lipofectamine 2000 and Opti-MEM media according to the manufacturer's protocol. Extracellular DNA was removed by a full media change the next day and virus-containing media was harvested 48 hours after. Lentiviral media was concentrated using Lenti-X Concentrator (TaKaRa Biosciences) and 5 mL lentiviral aliquots were made and stored at −80° C. Lentiviral titering was performed by enumerating colony forming units post Puromycin selection. HEK293T or U2OS cells carrying a BFP-expressing genomic landing pad were seeded at 6×10⁷ cells in culture plates and transduced at a 0.3 multiplicity of infection (MOI) to minimize multiple infections per cell. Puromycin (2.5 μg/mL) was added 48 hours post infection to allow for selection of infected cells. Cells were kept under puromycin selection for at least 7 days and then scaled up for tgRNA electroporation.

To determine the genome-editing capacity of the gene modifying library candidates in the assay, infected BFP-expressing HEK293T or U2OS cells were then transfected by electroporation of 250,000 cells/well with 200 ng of a tgRNA (either g4 or g10) plasmid, designed to convert BFP to GFP, at sufficient cell count for >1000× coverage per library candidate.

The g4 tgRNA (5′ to 3′) is as follows: 20 nucleotide spacer region (GCCGAAGCACTGCACGCCGT; SEQ ID NO: 11,011), a scaffold region (GTTTTAGAGCTAGAAATAGCAAGTTAAAATAAGGCTAGTCCGTTATCAACTTGAAA AAGTGGCACCGAGTCGGTGC; SEQ ID NO: 11,012), the template region encoding the single base pair substitution to change BFP to GFP (bold) and a PAM inactivation that introduces a synonymous point mutation in the SpyCas9 PAM (NGG to NCG) that prevents re-engagement of the heterologous gene modifying polypeptide upon completion of a functional gene modifying reaction (underline) (ACCCTGACGTACG; SEQ ID NO: 11,013), and the 13 nucleotide PBS (GCGTGCAGTGCTT; SEQ ID NO: 11,014).

Similarly, the g10 tgRNA (5′ to 3′) is as follows: 20 nucleotide spacer region (AGAAGTCGTGCTGCTTCATG; SEQ ID NO: 11,015), a scaffold region (GTTTTAGAGCTAGAAATAGCAAGTTAAAATAAGGCTAGTCCGTTATCAACTTGAAA AAGTGGCACCGAGTCGGTGC; SEQ ID NO: 11,016), the template region encoding the single base pair substitution to change BFP to GFP (bold) and a PAM inactivation that introduces a synonymous point mutation in the SpyCas9 PAM (NGG to NGA) that prevents re-engagement of the heterologous gene modifying polypeptide upon completion of a functional gene modifying reaction (underline)

(ACCCTGACCTACGGCGTGCAGTGCTTCGGCCGCTACCCCGATCACAT;
SEQ ID NO: 11,017),
and
13 nucleotide PBS
(GAAGCAGCACGAC; SEQ ID NO: 11,018).

To assess the genome-editing capacity of the various constructs in the assay, cells were sorted by Fluorescence-Activated Cell Sorting (FACS) for GFP expression 6-7 days post-electroporation. Cells were sorted and harvested as distinct populations of unedited (BFP+) cells, edited (GFP+) cells and imperfect edit (BFP−, GFP−) cells. A sample of unsorted cells was also harvested as the input population to determine enrichment during analysis.

To determine which gene modifying library candidates have genome-editing capacity in this assay, genomic DNA (gDNA) was harvested from sorted and unsorted cell populations, and analyzed by sequencing the gene modifying library candidates in each population. Briefly, gene modifying sequences were amplified from the genome using primers specific to the lentiviral cassette, amplified in a second round of PCR to dilute genomic DNA, and then sequenced using Oxford Nanopore Sequencing Technology according to the manufacturer's protocol.

After quality control of sequencing reads, reads of at least 1500 and no more than 3200 nucleotides were mapped to the heterologous gene modifying polypeptide library sequences and those containing a minimum of an 80% match to a library sequence were considered to be successfully aligned to a given candidate. To identify gene modifying candidates capable of performing gene editing in the assay, the read count of each library candidate in the edited population was compared to its read count in the initial, unsorted population. For purposes of this pooled screen, gene modifying candidates with genome-editing capacity were selected as those candidates that were enriched in the converted (GFP+) population relative to unsorted (input) cells and wherein the enrichment was determined to be at or above the enrichment level of a reference (Element ID No: 17380).

A large number of heterologous gene modifying polypeptide candidates were determined to be enriched in the GFP+ cell populations. For example, of the 17,446 candidates tested, over 3,300 exhibited enrichment in GFP+ sorted populations (relative to unsorted) that was at least equivalent to that of the reference under similar experimental conditions (HEK293T using g4 tgRNA; HEK293T cells using g10 tgRNA; or U2OS cells using g4 tgRNA), shown in Table D. Although the 17,446 candidates were also tested in U2OS cells using g10 tgRNA, the pooled screen did not yield candidates that were enriched in the converted (GFP+) population relative to unsorted (input) cells under that experimental condition; further investigation is required to explain these results.

TABLE D
Combinations of linker and RT sequences screened. The amino acid sequence of
each RT in this table is provided in Table 6.

| | SEQ ID | |
|---|---|---|
| Linker amino acid sequence | NO: | RT domain name |
| EAAAKGSS | 12,001 | PERV_Q4VFZ2_3mutA_WS |
| EAAAKEAAAKEAAAKEAAAK | 12,002 | MLVMS_P03355_PLV919 |
| PAPEAAAK | 12,003 | MLVFF_P26809_3mutA |
| EAAAKPAPGGG | 12,004 | MLVFF_P26809_3mutA |
| GSSGSSGSSGSSGSSGSS | 12,005 | PERV_Q4VFZ2_3mut |
| PAPGGGEAAAK | 12,006 | MLVAV_P03356_3mutA |
| AEAAAKEAAAKEAAAKEAAAKALEAEAAAKEAAAKEAAAKEAAAKA | 12,007 | MLVMS_P03355_PLV919 |
| GSSEAAAK | 12,008 | MLVFF_P26809_3mutA |
| EAAAKPAPGGS | 12,009 | MLVFF_P26809_3mutA |
| GGSGGSGGSGGSGGSGGS | 12,010 | MLVFF_P26809_3mutA |
| AEAAAKEAAAKEAAAKEAAAKALEAEAAAKEAAAKEAAAKEAAAKA | 12,011 | XMRV6_A1Z651_3mutA |
| AEAAAKEAAAKEAAAKEAAAKALEAEAAAKEAAAKEAAAKEAAAKA | 12,012 | PERV_Q4VFZ2_3mutA_WS |
| EAAAKEAAAKEAAAK | 12,013 | MLVFF_P26809_3mutA |
| PAPEAAAKGSS | 12,014 | MLVFF_P26809_3mutA |
| AEAAAKEAAAKEAAAKEAAAKALEAEAAAKEAAAKEAAAKEAAAKA | 12,015 | PERV_Q4VFZ2_3mutA_WS |
| EAAAKEAAAKEAAAK | 12,016 | PERV_Q4VFZ2_3mutA_WS |
| AEAAAKEAAAKEAAAKEAAAKALEAEAAAKEAAAKEAAAKEAAAKA | 12,017 | AVIRE_P03360_3mutA |
| PAPAPAPAPAP | 12,018 | MLVCB_P08361_3mutA |
| PAPAPAPAPAP | 12,019 | MLVFF_P26809_3mutA |
| EAAAKGGSPAP | 12,020 | PERV_Q4VFZ2_3mutA_WS |
| PAP | | MLVMS_P03355_PLV919 |
| PAPGGGGSS | 12,022 | WMSV_P03359_3mutA |
| SGSETPGTSESATPES | 12,023 | MLVFF_P26809_3mutA |
| PAPEAAAKGSS | 12,024 | XMRV6_A1Z651_3mutA |
| EAAAKGGSGGG | 12,025 | MLVMS_P03355_PLV919 |
| GGGGSGGGGS | 12,026 | MLVFF_P26809_3mutA |
| GGGPAPGSS | 12,027 | MLVAV_P03356_3mutA |
| GGSGGSGGSGGSGGSGGS | 12,028 | XMRV6_A1Z651_3mut |
| GGGGSGGGGSGGGGSGGGGSGGGGSGGGGS | 12,029 | MLVCB_P08361_3mutA |
| GSSPAP | 12,030 | AVIRE_P03360_3mutA |
| EAAAKGSSPAP | 12,031 | MLVFF_P26809_3mutA |
| GSSGGGEAAAK | 12,032 | MLVFF_P26809_3mutA |
| GGSGGSGGSGGSGGSGGS | 12,033 | MLVMS_P03355_3mutA_WS |
| PAPAPAPAP | 12,034 | MLVFF_P26809_3mutA |
| EAAAKEAAAKEAAAKEAAAK | 12,035 | XMRV6_A1Z651_3mutA |
| EAAAKGGSPAP | 12,036 | MLVMS_P03355_3mutA_WS |
| PAPGGSEAAAK | 12,037 | AVIRE_P03360_3mutA |
| GGGGSGGGGSGGGGSGGGGSGGGGSGGGGS | 12,038 | AVIRE_P03360_3mutA |
| EAAAKGGGGSEAAAK | 12,039 | MLVCB_P08361_3mutA |
| AEAAAKEAAAKEAAAKEAAAKALEAEAAAKEAAAKEAAAKEAAAKA | 12,040 | WMSV_P03359_3mutA |
| GSS | | MLVMS_P03355_PLV919 |
| GSSGSSGSSGSS | 12,042 | MLVMS_P03355_PLV919 |
| GSSPAPEAAAK | 12,043 | XMRV6_A1Z651_3mutA |
| GGSPAPEAAAK | 12,044 | MLVFF_P26809_3mutA |
| GGGEAAAKGGS | 12,045 | MLVFF_P26809_3mutA |
| EAAAKEAAAKEAAAKEAAAKEAAAK | 12,046 | PERV_Q4VFZ2_3mutA_WS |
| GGGGGGGG | 12,047 | PERV_Q4VFZ2_3mut |
| GGGPAP | 12,048 | MLVCB_P08361_3mutA |
| PAPAPAPAPAPAP | 12,049 | MLVCB_P08361_3mutA |
| GGSGGSGGSGGSGGSGGS | 12,050 | MLVCB_P08361_3mutA |
| PAP | | MLVMS_P03355_3mutA_WS |
| GGSGGSGGSGGSGGSGGS | 12,052 | PERV_Q4VFZ2_3mutA_WS |
| PAPAPAPAPAPAP | 12,053 | MLVMS_P03355_PLV919 |
| EAAAKPAPGSS | 12,054 | MLVMS_P03355_3mutA_WS |
| EAAAKEAAAKEAAAKEAAAK | 12,055 | MLVMS_P03355_3mutA_WS |
| EAAAKGGS | 12,056 | MLVMS_P03355_3mutA_WS |
| GGGGSEAAAKGGGGS | 12,057 | MLVFF_P26809_3mutA |
| EAAAKPAPGSS | 12,058 | MLVFF_P26809_3mutA |
| GGGGSGGGGSGGGGSGGGGS | 12,059 | MLVMS_P03355_PLV919 |
| EAAAKGGGGGS | 12,060 | MLVMS_P03355_PLV919 |
| GGSPAP | 12,061 | XMRV6_A1Z651_3mutA |
| EAAAKGGGPAP | 12,062 | MLVMS_P03355_PLV919 |
| EAAAKEAAAKEAAAKEAAAKEAAAK | 12,063 | MLVFF_P26809_3mutA |
| PAP | | MLVCB_P08361_3mutA |
| EAAAK | 12,065 | XMRV6_A1Z651_3mutA |
| GGSGSSPAP | 12,066 | PERV_Q4VFZ2_3mutA_WS |
| GSSGSSGSSGSSGSSGSS | 12,067 | MLVMS_P03355_PLV919 |
| GSSEAAAKGGG | 12,068 | MLVAV_P03356_3mutA |
| GGGEAAAKGGS | 12,069 | XMRV6_A1Z651_3mutA |
| EAAAKGGGGSEAAAK | 12,070 | MLVAV_P03356_3mutA |
| GGGGSGGGGSGGGGS | 12,071 | MLVFF_P26809_3mutA |
| GGGGSGGGGSGGGGGGGGS | 12,072 | AVIRE_P03360_3mutA |
| SGSETPGTSESATPES | 12,073 | AVIRE_P03360_3mutA |
| GGGEAAAKPAP | 12,074 | MLVFF_P26809_3mutA |
| EAAAKGSSGGG | 12,075 | MLVMS_P03355_3mutA_WS |
| EAAAKEAAAKEAAAKEAAAKEAAAK | 12,076 | WMSV_P03359_3mut |
| GGSGGSGGSGGS | 12,077 | XMRV6_A1Z651_3mutA |
| GGSEAAAKPAP | 12,078 | MLVFF_P26809_3mutA |
| EAAAKGSSGGG | 12,079 | XMRV6_A1Z651_3mutA |
| GGGGS | 12,080 | MLVFF_P26809_3mutA |
| GGGEAAAKGSS | 12,081 | MLVMS_P03355_PLV919 |
| PAPAPAPAPAPAP | 12,082 | MLVAV_P03356_3mutA |
| GGGGSGGGGSGGGGSGGGGS | 12,083 | MLVCB_P08361_3mutA |
| GGGEAAAKGSS | 12,084 | MLVCB_P08361_3mutA |
| PAPGGSGSS | 12,085 | MLVFF_P26809_3mutA |
| GSAGSAAGSGEF | 12,086 | MLVCB_P08361_3mutA |
| PAPGGSEAAAK | 12,087 | MLVMS_P03355_3mutA_WS |
| GGSGSS | 12,088 | XMRV6_A1Z651_3mutA |
| PAPGGGGSS | 12,089 | MLVMS_P03355_PLV919 |
| GSSGSSGSS | 12,090 | XMRV6_A1Z651_3mut |
| AEAAAKEAAAKEAAAKEAAAKALEAEAAAKEAAAKEAAAKEAAAKA | 12,091 | MLVMS_P03355_3mutA_WS |
| EAAAK | 12,092 | MLVMS_P03355_PLV919 |
| GSSGSSGSSGSS | 12,093 | MLVFF_P26809_3mutA |
| PAPGGGGSS | 12,094 | MLVCB_P08361_3mutA |
| GGGEAAAKGGS | 12,095 | MLVCB_P08361_3mutA |
| PAPGGGEAAAK | 12,096 | MLVMS_P03355_PLV919 |
| GGGGGSPAP | 12,097 | XMRV6_A1Z651_3mutA |
| EAAAKGGS | 12,098 | XMRV6_A1Z651_3mutA |
| EAAAKGSSPAP | 12,099 | XMRV6_A1Z651_3mut |
| PAPEAAAK | 12,100 | MLVAV_P03356_3mutA |
| GGSGGSGGSGGS | 12,101 | MLVMS_P03355_3mutA_WS |
| GGGPAPGGS | 12,102 | MLVMS_P03355_PLV919 |
| GSSGSSGSSGSS | 12,103 | PERV_Q4VFZ2_3mutA_WS |
| EAAAKPAPGGS | 12,104 | MLVCB_P08361_3mutA |
| GSSGSS | 12,105 | MLVFF_P26809_3mutA |
| EAAAKEAAAKEAAAKEAAAK | 12,106 | MLVCB_P08361_3mutA |
| EAAAKEAAAKEAAAKEAAAK | 12,107 | FLV_P10273_3mutA |
| GSS | | MLVFF_P26809_3mutA |
| EAAAKEAAAK | 12,109 | MLVMS_P03355_3mutA_WS |
| PAPEAAAKGGG | 12,110 | MLVAV_P03356_3mutA |
| GGSGSSEAAAK | 12,111 | MLVFF_P26809_3mutA |
| EAAAKEAAAKEAAAKEAAAKEAAAK | 12,112 | PERV_Q4VFZ2 |
| GSSEAAAKPAP | 12,113 | AVIRE_P03360_3mutA |
| EAAAKEAAAKEAAAKEAAAKEAAAK | 12,114 | MLVCB_P08361_3mutA |
| EAAAKGGG | 12,115 | MLVFF_P26809_3mutA |
| GSSPAPGGG | 12,116 | MLVCB_P08361_3mutA |
| GGGPAPGSS | 12,117 | MLVMS_P03355_PLV919 |
| GGGGGS | 12,118 | MLVMS_P03355_3mutA_WS |
| EAAAKEAAAKEAAAKEAAAKEAAAKEAAAK | 12,119 | PERV_Q4VFZ2_3mut |
| GGGGSGGGGSGGGGSGGGGSGGGGS | 12,120 | WMSV_P03359_3mutA |
| EAAAKEAAAKEAAAK | 12,121 | PERV_Q4VFZ2_3mut |
| PAPAPAPAP | 12,122 | MLVCB_P08361_3mutA |
| GSSGSSGSSGSSGSS | 12,123 | PERV_Q4VFZ2_3mut |
| GGGGSSEAAAK | 12,124 | MLVMS_P03355_3mutA_WS |
| GGSGGSGGSGGS | 12,125 | MLVCB_P08361_3mutA |
| PAPEAAAKGGS | 12,126 | MLVCB_P08361_3mutA |
| EAAAKEAAAKEAAAKEAAAKEAAAKEAAAK | 12,127 | MLVCB_P08361_3mutA |
| EAAAKGGGGSEAAAK | 12,128 | MLVMS_P03355_PLV919 |
| EAAAKGGGGSEAAAK | 12,129 | MLVMS_P03355_3mutA_WS |
| EAAAKGGGPAP | 12,130 | XMRV6_A1Z651_3mut |
| EAAAKEAAAKEAAAKEAAAKEAAAK | 12,131 | MLVMS_P03355_3mutA_WS |
| AEAAAKEAAAKEAAAKEAAAKALEAEAAAKEAAAKEAAAKEAAAKA | 12,132 | FLV_P10273_3mutA |
| GGSEAAAKGGG | 12,133 | MLVMS_P03355_3mutA_WS |
| GGGGSGGGGSGGGGSGGGGSGGGGSGGGGS | 12,134 | KORV_Q9TTC1-Pro_3mutA |
| GGGPAPGGS | 12,135 | MLVCB_P08361_3mutA |
| PAPAPAPAPAPAP | 12,136 | XMRV6_A1Z651_3mutA |
| GGSGSSGGG | 12,137 | XMRV6_A1Z651_3mutA |
| GGSGSSGGG | 12,138 | MLVCB_P08361_3mutA |
| GGGEAAAKGGS | 12,139 | MLVMS_P03355_3mutA_WS |
| EAAAK | 12,140 | MLVCB_P08361_3mutA |
| GGSPAPGSS | 12,141 | MLVMS_P03355_3mutA_WS |
| GGGGSSEAAAK | 12,142 | PERV_Q4VFZ2_3mut |
| PAPAPAPAPAP | 12,143 | MLVBM_Q7SVK7_3mut |
| EAAAKEAAAKEAAAKEAAAK | 12,144 | MLVAV_P03356_3mutA |
| GGGGGSGSS | 12,145 | MLVCB_P08361_3mutA |
| EAAAKGSSPAP | 12,146 | MLVMS_P03355_3mutA_WS |
| PAPAPAPAPAPAP | 12,147 | MLVMS_P03355_3mutA_WS |
| GSSGGGGGS | 12,148 | MLVMS_P03355_3mutA_WS |
| PAPGSSGGG | 12,149 | MLVMS_P03355_PLV919 |
| GGSGGGPAP | 12,150 | MLVCB_P08361_3mutA |
| GGGGGGG | 12,151 | MLVCB_P08361_3mutA |
| GSSGSSGSSGSSGSSGSS | 12,152 | MLVCB_P08361_3mutA |
| GGGPAPGGS | 12,153 | MLVFF_P26809_3mutA |
| EAAAKGGSGGG | 12,154 | PERV_Q4VFZ2_3mut |
| EAAAKGGGGSS | 12,155 | MLVMS_P03355_3mutA_WS |
| GSSGSSGSSGSSGSSGSS | 12,156 | MLVMS_P03355_3mut |
| GGGGSGGGGSGGGGSGGGGS | 12,157 | MLVBM_Q7SVK7_3mutA_WS |
| PAPAPAPAPAP | 12,158 | MLVMS_P03355_PLV919 |
| GGGEAAAKGGS | 12,159 | MLVMS_P03355_PLV919 |
| AEAAAKEAAAKEAAAKEAAAKALEAEAAAKEAAAKEAAAKEAAAKA | 12,160 | MLVMS_P03355_3mut |
| GSAGSAAGSGEF | 12,161 | MLVMS_P03355_3mutA_WS |
| GSSGSSGSSGSSGSS | 12,162 | MLVFF_P26809_3mutA |
| EAAAKGGSGSS | 12,163 | MLVFF_P26809_3mutA |
| PAPGGG | 12,164 | MLVFF_P26809_3mutA |
| GGGPAPGSS | 12,165 | XMRV6_A1Z651_3mutA |
| PAPEAAAKGGS | 12,166 | AVIRE_P03360_3mutA |
| PAPGGGEAAAK | 12,167 | MLVFF_P26809_3mut |
| GGGGSSEAAAK | 12,168 | MLVCB_P08361_3mutA |
| EAAAK | 12,169 | MLVMS_P03355_PLV919 |
| GGGGSGGGGSGGGGSGGGGSGGGGSGGGGS | 12,170 | BAEVM_P10272_3mutA |
| GGSGGGEAAAK | 12,171 | MLVMS_P03355_PLV919 |
| AEAAAKEAAAKEAAAKEAAAKALEAEAAAKEAAAKEAAAKEAAAKA | 12,172 | MLVFF_P26809_3mutA |
| GSSPAPGGS | 12,173 | XMRV6_A1Z651_3mutA |
| GGSGGGPAP | 12,174 | MLVMS_P03355_PLV919 |
| EAAAK | 12,175 | AVIRE_P03360_3mutA |
| GSS | | XMRV6_A1Z651_3mutA |
| GGSGGSGGS | 12,177 | MLVFF_P26809_3mutA |
| EAAAKEAAAKEAAAKEAAAK | 12,178 | AVIRE_P03360_3mut |
| PAPEAAAKGGG | 12,179 | PERV_Q4VFZ2_3mutA_WS |
| GGGGGSEAAAK | 12,180 | BAEVM_P10272_3mutA |
| GGSGSSGGG | 12,181 | MLVMS_P03355_3mutA_WS |
| GGGGGGG | 12,182 | MLVMS_P03355_3mutA_WS |
| GSSEAAAKPAP | 12,183 | PERV_Q4VFZ2_3mut |
| GGGGGSEAAAK | 12,184 | WMSV_P03359_3mut |
| GGGGSGGGGSGGGGSGGGGSGGGGS | 12,185 | MLVFF_P26809_3mut |
| GGGEAAAKGGS | 12,186 | AVIRE_P03360_3mutA |
| GGSPAPGGG | 12,187 | AVIRE_P03360_3mutA |
| GSAGSAAGSGEF | 12,188 | MLVAV_P03356_3mutA |
| EAAAK | 12,189 | MLVAV_P03356_3mutA |
| EAAAKPAPGSS | 12,190 | WMSV_P03359_3mutA |
| EAAAKEAAAKEAAAKEAAAKEAAAKEAAAK | 12,191 | PERV_Q4VFZ2_3mutA_WS |
| GGSEAAAKPAP | 12,192 | MLVCB_P08361_3mutA |
| PAPAPAPAPAPAP | 12,193 | MLVBM_Q7SVK7_3mutA_WS |
| GGSPAPGGG | 12,194 | MLVMS_P03355_3mutA_WS |
| GGSEAAAKGGG | 12,195 | MLVMS_P03355_3mut |
| GGSGGSGGSGGS | 12,196 | MLVFF_P26809_3mutA |
| EAAAKEAAAKEAAAKEAAAKEAAAKEAAAK | 12,197 | MLVFF_P26809_3mutA |
| GGG | | AVIRE_P03360_3mutA |
| AEAAAKEAAAKEAAAKEAAAKALEAEAAAKEAAAKEAAAKEAAAKA | 12,199 | PERV_Q4VFZ2_3mut |
| GGSGGSGGSGGS | 12,200 | MLVMS_P03355_3mutA_WS |
| GGGEAAAK | 12,201 | MLVCB_P08361_3mutA |
| GSSGSSGSSGSSGSSGSS | 12,202 | MLVMS_P03355_3mutA_WS |
| GSSGGGPAP | 12,203 | MLVMS_P03355_3mutA_WS |
| GSSEAAAKPAP | 12,204 | MLVFF_P26809_3mutA |
| EAAAKEAAAK | 12,205 | MLVMS_P03355_PLV919 |
| GGGGSGGGGGGGGSGGGGSGGGGSGGGGS | 12,206 | MLVCB_P08361_3mut |
| GGGGGG | 12,207 | MLVMS_P03355_3mutA_WS |
| GGSGSSGGG | 12,208 | MLVFF_P26809_3mutA |
| GSSGGGEAAAK | 12,209 | PERV_Q4VFZ2_3mutA_WS |
| PAPAPAPAPAP | 12,210 | PERV_Q4VFZ2_3mut |
| EAAAKEAAAKEAAAKEAAAKEAAAKEAAAK | 12,211 | SFV3L_P27401_2mut |
| EAAAKGGSGGG | 12,212 | BAEVM_P10272_3mutA |
| GGGGSSPAP | 12,213 | PERV_Q4VFZ2_3mutA_WS |
| GGGEAAAKPAP | 12,214 | MLVMS_P03355_PLV919 |
| GGSGGGPAP | 12,215 | BAEVM_P10272_3mutA |
| PAPGSSGGS | 12,216 | MLVMS_P03355_PLV919 |
| GGSGGGPAP | 12,217 | MLVMS_P03355_3mutA_WS |
| EAAAKGGSPAP | 12,218 | PERV_Q4VFZ2_3mutA_WS |
| EAAAKGGSGGG | 12,219 | MLVMS_P03355_3mutA_WS |
| PAPGSSGGG | 12,220 | MLVFF_P26809_3mutA |
| GSSEAAAKGGS | 12,221 | MLVFF_P26809_3mutA |
| PAPGSSEAAAK | 12,222 | MLVFF_P26809_3mutA |
| EAAAKGSSPAP | 12,223 | KORV_Q9TTC1-Pro_3mutA |
| EAAAKEAAAKEAAAKEAAAKEAAAK | 12,224 | MLVBM_Q7SVK7_3mutA_WS |
| PAPGSSEAAAK | 12,225 | MLVMS_P03355_PLV919 |
| EAAAKGSSGGG | 12,226 | MLVMS_P03355_3mutA_WS |
| EAAAKGGGGGS | 12,227 | AVIRE_P03360_3mutA |
| EAAAKEAAAKEAAAK | 12,228 | MLVMS_P03355_PLV919 |
| PAPAPAPAPAPAP | 12,229 | MLVFF_P26809_3mutA |
| GGGGSGGGGSGGGGS | 12,230 | MLVCB_P08361_3mutA |
| PAPGGSEAAAK | 12,231 | MLVCB_P08361_3mutA |
| PAPGSSEAAAK | 12,232 | MLVBM_Q7SVK7_3mutA_WS |
| PAPEAAAKGSS | 12,233 | AVIRE_P03360_3mutA |
| GGSPAPGSS | 12,234 | WMSV_P03359_3mutA |
| PAPGGSGGG | 12,235 | MLVMS_P03355_PLV919 |
| EAAAKGGSGSS | 12,236 | MLVMS_P03355_3mutA_WS |
| GGSGGG | 12,237 | MLVFF_P26809_3mutA |
| GGSEAAAKGSS | 12,238 | KORV_Q9TTC1_3mutA |
| AEAAAKEAAAKEAAAKEAAAKALEAEAAAKEAAAKEAAAKEAAAKA | 12,239 | MLVCB_P08361_3mutA |
| PAPAPAPAPAPAP | 12,240 | PERV_Q4VFZ2_3mutA_WS |
| PAPEAAAK | 12,241 | MLVMS_P03355_3mutA_WS |
| GGSEAAAKGGG | 12,242 | MLVMS_P03355_PLV919 |
| GSSPAP | 12,243 | MLVMS_P03355_3mutA_WS |
| GGGGSS | 12,244 | MLVMS_P03355_PLV919 |
| GGGEAAAKPAP | 12,245 | AVIRE_P03360_3mutA |
| EAAAKPAPGGS | 12,246 | MLVAV_P03356_3mutA |
| EAAAKGGGPAP | 12,247 | MLVAV_P03356_3mutA |
| PAPGGSEAAAK | 12,248 | BAEVM_P10272_3mutA |
| PAPGGSGSS | 12,249 | MLVMS_P03355_3mutA_WS |
| PAPGGSGSS | 12,250 | AVIRE_P03360_3mutA |
| GGSGGGPAP | 12,251 | MLVMS_P03355_3mutA_WS |
| EAAAKEAAAKEAAAKEAAAK | 12,252 | BAEVM_P10272_3mutA |
| GGGGSGGGGSGGGGSGGGGSGGGGS | 12,253 | MLVMS_P03355_PLV919 |
| GGGGSSPAP | 12,254 | MLVCB_P08361_3mutA |
| GSSGGGPAP | 12,255 | MLVFF_P26809_3mutA |
| GGGGSSGGS | 12,256 | MLVMS_P03355_PLV919 |
| GGSGGG | 12,257 | MLVCB_P08361_3mutA |
| GSSGGGGGS | 12,258 | MLVMS_P03355_PLV919 |
| SGGSSGGSSGSETPGTSESATPESSGGSSGGSS | 12,259 | XMRV6_A1Z651_3mutA |
| GGGGGSGSS | 12,260 | KORV_Q9TTC1_3mut |
| GGGEAAAKGGS | 12,261 | BAEVM_P10272_3mutA |
| GGSGGG | 12,262 | BAEVM_P10272_3mutA |
| PAPAPAP | 12,263 | KORV_Q9TTC1-Pro_3mut |
| AEAAAKEAAAKEAAAKEAAAKALEAEAAAKEAAAKEAAAKEAAAKA | 12,264 | SFV3L_P27401_2mutA |
| AEAAAKEAAAKEAAAKEAAAKALEAEAAAKEAAAKEAAAKEAAAKA | 12,265 | MLVBM_Q7SVK7_3mutA_WS |
| GSSGSSGSSGSSGSS | 12,266 | MLVMS_P03355_3mutA_WS |
| GSSGGGEAAAK | 12,267 | MLVMS_P03355_3mutA_WS |
| GSSGGSEAAAK | 12,268 | MLVFF_P26809_3mutA |
| PAP | | MLVMS_P03355_PLV919 |
| EAAAKGGGGSEAAAK | 12,270 | MLVBM_Q7SVK7_3mutA_WS |
| PAPAP | 12,271 | AVIRE_P03360_3mutA |
| PAP | | MLVFF_P26809_3mutA |
| GSSGGG | 12,273 | MLVMS_P03355_3mut |
| GSSPAPGGS | 12,274 | MLVFF_P26809_3mutA |
| PAPAPAPAP | 12,275 | XMRV6_A1Z651_3mutA |
| EAAAKGSSGGS | 12,276 | PERV_Q4VFZ2_3mut |
| PAPEAAAKGGG | 12,277 | KORV_Q9TTC1-Pro_3mutA |
| PAPGGS | 12,278 | MLVCB_P08361_3mutA |
| EAAAKGGG | 12,279 | MLVCB_P08361_3mutA |
| GSSEAAAKPAP | 12,280 | MLVMS_P03355_PLV919 |
| PAPGGS | 12,281 | MLVFF_P26809_3mutA |
| EAAAKGGS | 12,282 | MLVCB_P08361_3mutA |
| EAAAKEAAAKEAAAKEAAAKEAAAKEAAAK | 12,283 | FLV_P10273_3mutA |
| PAPGGSEAAAK | 12,284 | MLVAV_P03356_3mutA |
| GSS | | MLVCB_P08361_3mutA |
| GSSGSSGSSGSS | 12,286 | AVIRE_P03360_3mutA |
| GSSGSSGSS | 12,287 | MLVFF_P26809_3mutA |
| GSSGGG | 12,288 | MLVMS_P03355_PLV919 |
| EAAAK | 12,289 | MLVFF_P26809_3mutA |
| GGSPAPEAAAK | 12,290 | MLVCB_P08361_3mutA |
| GGSGSS | 12,291 | MLVCB_P08361_3mutA |
| GSSPAPGGG | 12,292 | MLVMS_P03355_PLV919 |
| EAAAKEAAAKEAAAKEAAAKEAAAK | 12,293 | MLVAV_P03356_3mutA |
| EAAAKGSSPAP | 12,294 | FLV_P10273_3mutA |
| GGGGSS | 12,295 | XMRV6_A1Z651_3mutA |
| GGSPAPGSS | 12,296 | MLVMS_P03355_PLV919 |
| EAAAKEAAAKEAAAKEAAAKEAAAK | 12,297 | MLVMS_P03355_3mutA_WS |
| PAPEAAAKGGG | 12,298 | FLV_P10273_3mutA |
| EAAAKPAPGGS | 12,299 | XMRV6_A1Z651_3mut |
| PAPAP | 12,300 | BAEVM_P10272_3mutA |
| EAAAKEAAAKEAAAKEAAAK | 12,301 | MLVMS_P03355_PLV919 |
| GSSPAPGGG | 12,302 | MLVMS_P03355_PLV919 |
| EAAAKGGGPAP | 12,303 | KORV_Q9TTC1_3mutA |
| PAPEAAAK | 12,304 | MLVMS_P03355_PLV919 |
| PAPGGGEAAAK | 12,305 | PERV_Q4VFZ2_3mutA_WS |
| EAAAKGSSGGS | 12,306 | MLVMS_P03355_3mutA_WS |
| EAAAKEAAAKEAAAK | 12,307 | MLVMS_P03355_PLV919 |
| GSSEAAAK | 12,308 | MLVMS_P03355_3mutA_WS |
| GSSGSSGSSGSS | 12,309 | MLVMS_P03355_3mutA_WS |
| GGGGSGGGGSGGGGSGGGGS | 12,310 | MLVMS_P03355_3mutA_WS |
| EAAAKGGGGSEAAAK | 12,311 | MLVMS_P03355_3mut |
| GGS | | MLVCB_P08361_3mutA |
| GGGGSGGGGSGGGGSGGGGSGGGGSGGGGS | 12,313 | XMRV6_A1Z651_3mutA |
| GGSGSSPAP | 12,314 | MLVCB_P08361_3mutA |
| GGGGGGGGSGGGGS | 12,315 | XMRV6_A1Z651_3mutA |
| PAPAPAPAPAP | 12,316 | BAEVM_P10272_3mutA |
| PAPAPAPAPAP | 12,317 | MLVMS_P03355_3mutA_WS |
| EAAAKEAAAKEAAAKEAAAK | 12,318 | MLVBM_Q7SVK7_3mut |
| GGGGSGGGGSGGGGSGGGGSGGGGS | 12,319 | BAEVM_P10272_3mutA |
| GGSGGSGGS | 12,320 | MLVMS_P03355_3mutA_WS |
| EAAAKPAPGSS | 12,321 | MLVMS_P03355_PLV919 |
| GSS | | MLVMS_P03355_3mutA_WS |
| PAPEAAAKGGS | 12,323 | MLVMS_P03355_3mutA_WS |
| GGGPAPGGS | 12,324 | MLVMS_P03355_3mutA_WS |
| EAAAKGGGGSS | 12,325 | MLVAV_P03356_3mutA |
| GSSGSSGSSGSSGSS | 12,326 | MLVFF_P26809_3mut |
| SGSETPGTSESATPES | 12,327 | PERV_Q4VFZ2_3mut |
| GGSEAAAKGGG | 12,328 | MLVMS_P03355_3mut |
| GSSGSSGSSGSSGSSGSS | 12,329 | AVIRE_P03360_3mutA |
| PAPAPAPAPAPAP | 12,330 | AVIRE_P03360_3mut |
| GGSGGS | 12,331 | XMRV6_A1Z651_3mutA |
| PAPGSSEAAAK | 12,332 | MLVCB_P08361_3mut |
| GGSPAPEAAAK | 12,333 | PERV_Q4VFZ2_3mut |
| EAAAKGGGGGS | 12,334 | MLVCB_P08361_3mutA |
| GGSGGSGGSGGS | 12,335 | MLVMS_P03355_PLV919 |
| GGGGSSEAAAK | 12,336 | MLVMS_P03355_PLV919 |
| GSSEAAAKGGG | 12,337 | MLVFF_P26809_3mutA |
| PAPGGS | 12,338 | MLVMS_P03355_3mutA_WS |
| EAAAKGGSGGG | 12,339 | MLVCB_P08361_3mutA |
| EAAAKGGG | 12,340 | PERV_Q4VFZ2_3mut |
| PAPGGS | 12,341 | XMRV6_A1Z651_3mutA |
| GSSPAPGGG | 12,342 | XMRV6_A1Z651_3mutA |
| PAPEAAAKGGG | 12,343 | MLVMS_P03355_3mutA_WS |
| GSSEAAAKGGG | 12,344 | PERV_Q4VFZ2_3mutA_WS |
| PAPGGSEAAAK | 12,345 | XMRV6_A1Z651_3mutA |
| GGGGGS | 12,346 | MLVMS_P03355_3mutA_WS |
| GGSPAPEAAAK | 12,347 | MLVMS_P03355_3mutA_WS |
| GGGPAP | 12,348 | MLVFF_P26809_3mutA |
| PAPGSSGGG | 12,349 | XMRV6_A1Z651_3mutA |
| PAPGSSGGG | 12,350 | MLVBM_Q7SVK7_3mutA_WS |
| GGGEAAAKGSS | 12,351 | MLVMS_P03355_3mutA_WS |
| GSSEAAAKGGS | 12,352 | MLVCB_P08361_3mutA |
| PAPGGSGSS | 12,353 | MLVCB_P08361_3mutA |
| EAAAKGGGGSEAAAK | 12,354 | BAEVM_P10272_3mutA |
| PAPAPAP | 12,355 | PERV_Q4VFZ2_3mutA_WS |
| GGGGGG | 12,356 | MLVAV_P03356_3mutA |
| GSSPAPEAAAK | 12,357 | MLVCB_P08361_3mutA |
| GGSGGSGGS | 12,358 | MLVMS_P03355_3mutA_WS |
| GSSGSSGSSGSSGSS | 12,359 | XMRV6_A1Z651_3mut |
| GGGPAPGGS | 12,360 | XMRV6_A1Z651_3mutA |
| GGGPAPEAAAK | 12,361 | BAEVM_P10272_3mutA |
| GGSGGG | 12,362 | AVIRE_P03360_3mutA |
| SGSETPGTSESATPES | 12,363 | PERV_Q4VFZ2_3mutA_WS |
| EAAAKGSSPAP | 12,364 | MLVMS_P03355_PLV919 |
| GSSEAAAK | 12,365 | XMRV6_A1Z651_3mut |
| GSSGGSGGG | 12,366 | MLVFF_P26809_3mutA |
| EAAAKEAAAKEAAAKEAAAKEAAAK | 12,367 | WMSV_P03359_3mutA |
| GGGGSEAAAKGGGGS | 12,368 | MLVMS_P03355_PLV919 |
| PAPGGGGSS | 12,369 | MLVMS_P03355_3mutA_WS |
| SGSETPGTSESATPES | 12,370 | MLVMS_P03355_3mutA_WS |
| GGSPAPEAAAK | 12,371 | KORV_Q9TTC1-Pro_3mutA |
| GSSEAAAKGGG | 12,372 | MLVMS_P03355_3mutA_WS |
| GSSEAAAK | 12,373 | WMSV_P03359_3mutA |
| GGGGSEAAAKGGGGS | 12,374 | AVIRE_P03360_3mutA |
| GSS | | WMSV_P03359_3mutA |
| PAPGGSEAAAK | 12,376 | MLVFF_P26809_3mutA |
| GGGGS | 12,377 | MLVMS_P03355_3mutA_WS |
| GGGPAP | 12,378 | MLVMS_P03355_3mutA_WS |
| EAAAKEAAAKEAAAKEAAAKEAAAKEAAAK | 12,379 | MLVMS_P03355_3mutA_WS |
| EAAAKPAPGSS | 12,380 | PERV_Q4VFZ2_3mut |
| EAAAKPAPGSS | 12,381 | MLVCB_P08361_3mutA |
| GGGGGG | 12,382 | WMSV_P03359_3mutA |
| EAAAKPAPGGS | 12,383 | MLVMS_P03355_PLV919 |
| PAPGGGEAAAK | 12,384 | PERV_Q4VFZ2_3mut |
| EAAAKEAAAKEAAAKEAAAKEAAAK | 12,385 | AVIRE_P03360_3mutA |
| GSSEAAAKPAP | 12,386 | XMRV6_A1Z651_3mutA |
| PAPGGSEAAAK | 12,387 | MLVBM_Q7SVK7_3mutA_WS |
| PAPGSS | 12,388 | MLVCB_P08361_3mutA |
| EAAAKGGG | 12,389 | MLVMS_P03355_3mutA_WS |
| EAAAKPAP | 12,390 | MLVCB_P08361_3mutA |
| PAPEAAAKGGS | 12,391 | MLVBM_Q7SVK7_3mutA_WS |
| GGSPAPGGG | 12,392 | MLVCB_P08361_3mutA |
| PAPGGSGSS | 12,393 | WMSV_P03359_3mutA |
| EAAAKEAAAKEAAAKEAAAKEAAAKEAAAK | 12,394 | MLVMS_P03355_PLV919 |
| GGSGGGPAP | 12,395 | MLVMS_P03355_PLV919 |
| AEAAAKEAAAKEAAAKEAAAKALEAEAAAKEAAAKEAAAKEAAAKA | 12,396 | MLVMS_P03355 |
| PAPEAAAKGSS | 12,397 | MLVCB_P08361_3mutA |
| EAAAKGSS | 12,398 | MLVMS_P03355_3mutA_WS |
| GGSGGS | 12,399 | MLVMS_P03355_3mutA_WS |
| EAAAKEAAAKEAAAKEAAAKEAAAK | 12,400 | BAEVM_P10272_3mutA |
| GGGGSEAAAKGGGGS | 12,401 | FLV_P10273_3mutA |
| GGSEAAAKGGG | 12,402 | MLVCB_P08361_3mutA |
| GSSGSSGSSGSSGSS | 12,403 | BAEVM_P10272_3mutA |
| GGGGSGGGGSGGGGSGGGGSGGGGSGGGGS | 12,404 | MLVFF_P26809_3mutA |
| EAAAKGGG | 12,405 | PERV_Q4VFZ2_3mut |
| GGGGGSEAAAK | 12,406 | MLVCB_P08361_3mutA |
| EAAAKPAPGGS | 12,407 | MLVMS_P03355_3mutA_WS |
| GGGGGSGSS | 12,408 | XMRV6_A1Z651_3mutA |
| PAPGSSEAAAK | 12,409 | MLVMS_P03355_3mutA_WS |
| GSSEAAAKPAP | 12,410 | MLVCB_P08361_3mutA |
| EAAAKGSSPAP | 12,411 | MLVAV_P03356_3mutA |
| GGGPAPGGS | 12,412 | WMSV_P03359_3mutA |
| GGSPAP | 12,413 | MLVMS_P03355_3mutA_WS |
| GGSEAAAKGGG | 12,414 | MLVMS_P03355_3mutA_WS |
| GGGGGGGG | 12,415 | MLVFF_P26809_3mutA |
| GGGGSGGGGSGGGGSGGGGSGGGGSGGGGS | 12,416 | MLVMS_P03355_3mutA_WS |
| GGGGSGGGGSGGGGSGGGGSGGGGSGGGGS | 12,417 | MLVBM_Q7SVK7_3mutA_WS |
| GSSPAPGGG | 12,418 | MLVAV_P03356_3mutA |
| GGGGGG | 12,419 | AVIRE_P03360_3mutA |
| GSSGGS | 12,420 | MLVMS_P03355_3mutA_WS |
| GGSPAPGSS | 12,421 | MLVFF_P26809_3mutA |
| PAPEAAAKGGG | 12,422 | PERV_Q4VFZ2_3mut |
| EAAAKGGGPAP | 12,423 | MLVFF_P26809_3mutA |
| GGGEAAAKGGS | 12,424 | MLVMS_P03355_PLV919 |
| GGSGSSPAP | 12,425 | MLVFF_P26809_3mutA |
| SGSETPGTSESATPES | 12,426 | WMSV_P03359_3mutA |
| PAPGGSEAAAK | 12,427 | MLVBM_Q7SVK7_3mutA_WS |
| GGSGGG | 12,428 | MLVMS_P03355_PLV919 |
| GGGGSSPAP | 12,429 | PERV_Q4VFZ2_3mut |
| GGGEAAAKGSS | 12,430 | MLVAV_P03356_3mutA |
| PAPAPAPAPAPAP | 12,431 | MLVMS_P03355_3mutA_WS |
| EAAAKGGGGSEAAAK | 12,432 | PERV_Q4VFZ2 |
| EAAAKEAAAKEAAAKEAAAKEAAAK | 12,433 | MLVMS_P03355_PLV919 |
| GGGGGSEAAAK | 12,434 | PERV_Q4VFZ2_3mut |
| PAPGSSEAAAK | 12,435 | MLVCB_P08361_3mutA |
| GSAGSAAGSGEF | 12,436 | PERV_Q4VFZ2_3mutA_WS |
| EAAAKGGGGSEAAAK | 12,437 | MLVFF_P26809_3mutA |
| GGSPAPGGG | 12,438 | PERV_Q4VFZ2_3mutA_WS |
| GSSEAAAKGGG | 12,439 | AVIRE_P03360_3mutA |
| GGGEAAAKPAP | 12,440 | MLVMS_P03355_3mutA_WS |
| GGGPAP | 12,441 | AVIRE_P03360_3mutA |
| GGSEAAAK | 12,442 | MLVCB_P08361_3mutA |
| SGGSSGGSSGSETPGTSESATPESSGGSSGGSS | 12,443 | PERV_Q4VFZ2_3mut |
| EAAAKPAPGGS | 12,444 | MLVBM_Q7SVK7_3mutA_WS |
| AEAAAKEAAAKEAAAKEAAAKALEAEAAAKEAAAKEAAAKEAAAKA | 12,445 | XMRV6_A1Z651_3mut |
| GGGGGGGG | 12,446 | MLVCB_P08361_3mutA |
| PAPGSS | 12,447 | PERV_Q4VFZ2_3mut |
| EAAAK | 12,448 | PERV_Q4VFZ2_3mut |
| GSAGSAAGSGEF | 12,449 | MLVMS_P03355_3mutA_WS |
| PAPGGGEAAAK | 12,450 | PERV_Q4VFZ2_3mut |
| EAAAKGSSGGS | 12,451 | MLVFF_P26809_3mut |
| GGGGSEAAAKGGGGS | 12,452 | BAEVM_P10272_3mutA |
| GGGGSGGGGSGGGGS | 12,453 | MLVMS_P03355_PLV919 |
| EAAAKGGGGSEAAAK | 12,454 | BAEVM_P10272_3mut |
| PAPGGGEAAAK | 12,455 | MLVMS_P03355_3mutA_WS |
| GGSEAAAKPAP | 12,456 | MLVMS_P03355_3mutA_WS |
| PAPAP | 12,457 | MLVCB_P08361_3mutA |
| PAPAP | 12,458 | MLVFF_P26809_3mutA |
| GGSPAP | 12,459 | AVIRE_P03360_3mutA |
| EAAAKGSSGGS | 12,460 | MLVCB_P08361_3mutA |
| PAPGSSGGS | 12,461 | AVIRE_P03360_3mutA |
| EAAAKGGGGSEAAAK | 12,462 | XMRV6_A1Z651_3mutA |
| PAPAPAP | 12,463 | BAEVM_P10272_3mutA |
| GGSGGSGGSGGSGGSGGS | 12,464 | MLVMS_P03355_PLV919 |
| GGGGGSGSS | 12,465 | MLVMS_P03355_PLV919 |
| PAPGSSEAAAK | 12,466 | XMRV6_A1Z651_3mut |
| GGSEAAAKPAP | 12,467 | XMRV6_A1Z651_3mutA |
| EAAAKEAAAKEAAAKEAAAK | 12,468 | XMRV6_A1Z651_3mut |
| AEAAAKEAAAKEAAAKEAAAKALEAEAAAKEAAAKEAAAKEAAAKA | 12,469 | WMSV_P03359_3mut |
| GGSGGGEAAAK | 12,470 | XMRV6_A1Z651_3mutA |
| GGGEAAAK | 12,471 | XMRV6_A1Z651_3mutA |
| GGGGSGGGGSGGGGS | 12,472 | MLVMS_P03355_3mutA_WS |
| GGSGGSGGSGGSGGS | 12,473 | MLVFF_P26809_3mutA |
| GSSGGGGGS | 12,474 | MLVMS_P03355_3mut |
| PAPGGSEAAAK | 12,475 | MLVMS_P03355_3mutA_WS |
| GSSGGSPAP | 12,476 | MLVMS_P03355_3mutA_WS |
| SGSETPGTSESATPES | 12,477 | XMRV6_A1Z651_3mutA |
| GGGGSGGGGS | 12,478 | MLVMS_P03355_PLV919 |
| PAPAPAPAPAP | 12,479 | MLVMS_P03355_3mut |
| GSSGSS | 12,480 | XMRV6_A1Z651_3mutA |
| GSSEAAAKPAP | 12,481 | PERV_Q4VFZ2_3mut |
| GGSGSSGGG | 12,482 | MLVMS_P03355_3mutA_WS |
| EAAAKEAAAK | 12,483 | MLVCB_P08361_3mutA |
| GSSGSSGSSGSS | 12,484 | MLVMS_P03355_3mutA_WS |
| GSSPAPGGG | 12,485 | PERV_Q4VFZ2_3mutA_WS |
| EAAAKEAAAKEAAAK | 12,486 | MLVMS_P03355_3mutA_WS |
| AEAAAKEAAAKEAAAKEAAAKALEAEAAAKEAAAKEAAAKEAAAKA | 12,487 | SFV1_P23074_2mutA |
| GGGGSGGGGSGGGGSGGGGSGGGGSGGGGS | 12,488 | MLVMS_P03355_PLV919 |
| GSAGSAAGSGEF | 12,489 | MLVMS_P03355_PLV919 |
| PAPGSSEAAAK | 12,490 | MLVMS_P03355_3mutA_WS |
| GGSEAAAK | 12,491 | MLVMS_P03355_3mutA_WS |
| GSSGSSGSSGSSGSS | 12,492 | PERV_Q4VFZ2_3mutA_WS |
| GGSEAAAKPAP | 12,493 | PERV_Q4VFZ2_3mutA_WS |
| GGSGGSGGS | 12,494 | MLVCB_P08361_3mutA |
| EAAAKGGSGSS | 12,495 | MLVCB_P08361_3mutA |
| GGGGSGGGGSGGGGSGGGGSGGGGS | 12,496 | FLV_P10273_3mutA |
| EAAAKEAAAKEAAAKEAAAK | 12,497 | MLVBM_Q7SVK7_3mutA_WS |
| GGSGSSPAP | 12,498 | BAEVM_P10272_3mutA |
| EAAAKEAAAKEAAAKEAAAKEAAAK | 12,499 | XMRV6_A1Z651_3mutA |
| GGGGSGGGGSGGGGSGGGGSGGGGS | 12,500 | MLVBM_Q7SVK7_3mutA_WS |
| GGSGSS | 12,501 | WMSV_P03359_3mutA |
| PAPEAAAK | 12,502 | MLVCB_P08361_3mutA |
| EAAAKPAP | 12,503 | BAEVM_P10272_3mutA |
| GSSPAP | 12,504 | PERV_Q4VFZ2_3mutA_WS |
| GGGPAP | 12,505 | PERV_Q4VFZ2_3mutA_WS |
| EAAAKGGSGSS | 12,506 | MLVMS_P03355_3mutA_WS |
| EAAAKGGGGSEAAAK | 12,507 | AVIRE_P03360_3mutA |
| GGSGGG | 12,508 | KORV_Q9TTC1-Pro_3mutA |
| GSSPAP | 12,509 | MLVFF_P26809_3mutA |
| GGSGSSEAAAK | 12,510 | BAEVM_P10272_3mutA |
| PAPGSSGGS | 12,511 | BAEVM_P10272_3mutA |
| GGGGGG | 12,512 | MLVFF_P26809_3mutA |
| PAPGGSEAAAK | 12,513 | MLVMS_P03355_PLV919 |
| PAPGGS | 12,514 | MLVMS_P03355_PLV919 |
| GGSGGSGGSGGS | 12,515 | BAEVM_P10272_3mutA |
| GSSPAP | 12,516 | MLVCB_P08361_3mutA |
| PAPAPAPAP | 12,517 | MLVMS_P03355_3mutA_WS |
| GGGGGG | 12,518 | MLVCB_P08361_3mutA |
| GSSGSSGSSGSSGSSGSS | 12,519 | KORV_Q9TTC1-Pro_3mutA |
| GSSEAAAKGGS | 12,520 | BAEVM_P10272_3mutA |
| GGSEAAAK | 12,521 | FLV_P10273_3mutA |
| GGSGGSGGSGGSGGS | 12,522 | KORV_Q9TTC1-Pro_3mutA |
| GSSPAPEAAAK | 12,523 | PERV_Q4VFZ2_3mut |
| GSSGSSGSSGSSGSS | 12,524 | XMRV6_A1Z651_3mutA |
| EAAAKPAPGGS | 12,525 | MLVMS_P03355_3mut |
| SGGSSGGSSGSETPGTSESATPESSGGSSGGSS | 12,526 | FLV_P10273_3mut |
| GGSPAPEAAAK | 12,527 | XMRV6_A1Z651_3mut |
| EAAAKGGSGGG | 12,528 | MLVFF_P26809_3mutA |
| EAAAKEAAAKEAAAKEAAAK | 12,529 | MLVFF_P26809_3mutA |
| GSSPAP | 12,530 | WMSV_P03359_3mutA |
| PAPAPAPAP | 12,531 | MLVAV_P03356_3mutA |
| PAPGGSEAAAK | 12,532 | KORV_Q9TTC1_3mut |
| GGSGSSEAAAK | 12,533 | MLVBM_Q7SVK7_3mutA_WS |
| GSSGGG | 12,534 | MLVCB_P08361_3mutA |
| GGGEAAAKGSS | 12,535 | PERV_Q4VFZ2_3mut |
| PAPGGSGGG | 12,536 | MLVFF_P26809_3mutA |
| AEAAAKEAAAKEAAAKEAAAKALEAEAAAKEAAAKEAAAKEAAAKA | 12,537 | FFV_093209 |
| PAPGGGGSS | 12,538 | MLVMS_P03355_3mutA_WS |
| EAAAKGGS | 12,539 | MLVAV_P03356_3mutA |
| EAAAKEAAAKEAAAKEAAAKEAAAKEAAAK | 12,540 | MLVBM_Q7SVK7_3mutA_WS |
| GGSGGSGGS | 12,541 | WMSV_P03359_3mutA |
| PAPAP | 12,542 | MLVMS_P03355_3mutA_WS |
| GSSGGGEAAAK | 12,543 | MLVAV_P03356_3mutA |
| GGGGSSEAAAK | 12,544 | MLVFF_P26809_3mutA |
| EAAAKGSSGGS | 12,545 | MLVMS_P03355_PLV919 |
| EAAAKGGGGSEAAAK | 12,546 | MLVMS_P03355_3mutA_WS |
| GGGGGGGG | 12,547 | MLVMS_P03355_PLV919 |
| GSSGSSGSS | 12,548 | MLVMS_P03355_PLV919 |
| GGGEAAAKPAP | 12,549 | PERV_Q4VFZ2_3mutA_WS |
| GGGGGSGSS | 12,550 | MLVMS_P03355_3mutA_WS |
| GGGGGGG | 12,551 | MLVMS_P03355_PLV919 |
| GGS | | MLVMS_P03355_PLV919 |
| GSSGGG | 12,553 | MLVMS_P03355_3mutA_WS |
| EAAAKGGSGSS | 12,554 | PERV_Q4VFZ2_3mutA_WS |
| PAPGSSEAAAK | 12,555 | MLVMS_P03355_PLV919 |
| GSSEAAAKPAP | 12,556 | MLVMS_P03355_PLV919 |
| GGSPAPGSS | 12,557 | BAEVM_P10272_3mutA |
| GSAGSAAGSGEF | 12,558 | MLVCB_P08361_3mut |
| GGSPAPGGG | 12,559 | PERV_Q4VFZ2_3mut |
| GGGGSGGGGSGGGGSGGGGS | 12,560 | MLVMS_P03355_3mut |
| GSSGSSGSS | 12,561 | PERV_Q4VFZ2_3mutA_WS |
| EAAAKEAAAKEAAAKEAAAKEAAAKEAAAK | 12,562 | PERV_Q4VFZ2_3mut |
| GGGGSEAAAKGGGGS | 12,563 | MLVCB_P08361_3mutA |
| GGSEAAAKGSS | 12,564 | MLVAV_P03356_3mutA |
| EAAAKGGGGSEAAAK | 12,565 | MLVCB_P08361_3mut |
| EAAAKEAAAKEAAAKEAAAKEAAAKEAAAK | 12,566 | XMRV6_A1Z651_3mutA |
| PAPGGGEAAAK | 12,567 | MLVMS_P03355_3mutA_WS |
| GSSGGGEAAAK | 12,568 | PERV_Q4VFZ2_3mutA_WS |
| GSSGSS | 12,569 | MLVCB_P08361_3mut |
| PAPAPAPAPAPAP | 12,570 | PERV_Q4VFZ2_3mut |
| GGSPAPGGG | 12,571 | MLVFF_P26809_3mutA |
| GGSGGSGGSGGSGGS | 12,572 | MLVCB_P08361_3mutA |
| EAAAKEAAAK | 12,573 | MLVFF_P26809_3mutA |
| AEAAAKEAAAKEAAAKEAAAKALEAEAAAKEAAAKEAAAKEAAAKA | 12,574 | GALV_P21414_3mut |
| PAPAPAPAPAPAP | 12,575 | WMSV_P03359_3mutA |
| GGGEAAAKGGS | 12,576 | KORV_Q9TTC1_3mutA |
| EAAAKGGGPAP | 12,577 | KORV_Q9TTC1_3mut |
| PAPEAAAKGSS | 12,578 | MLVBM_Q7SVK7_3mutA_WS |
| PAPEAAAKGSS | 12,579 | FLV_P10273_3mutA |
| PAPGGSEAAAK | 12,580 | MLVMS_P03355_3mut |
| GSSPAPGGG | 12,581 | BAEVM_P10272_3mutA |
| GGGEAAAKPAP | 12,582 | KORV_Q9TTC1-Pro_3mutA |
| GGGGSGGGGS | 12,583 | MLVMS_P03355_PLV919 |
| GGGEAAAKGSS | 12,584 | MLVFF_P26809_3mutA |
| PAPGGGGSS | 12,585 | MLVBM_Q7SVK7_3mutA_WS |
| GSSEAAAK | 12,586 | BAEVM_P10272_3mutA |
| GGGGGGGG | 12,587 | MLVMS_P03355_PLV919 |
| PAPGSSGGS | 12,588 | MLVAV_P03356_3mutA |
| GGGGSGGGGSGGGGSGGGGS | 12,589 | BAEVM_P10272_3mutA |
| PAP | | MLVMS_P03355_3mut |
| EAAAKGSSPAP | 12,591 | XMRV6_A1Z651_3mutA |
| PAPEAAAKGGS | 12,592 | MLVFF_P26809_3mutA |
| GSSGGGEAAAK | 12,593 | BAEVM_P10272_3mutA |
| PAPAPAP | 12,594 | MLVMS_P03355_3mutA_WS |
| GGSEAAAKGGG | 12,595 | MLVMS_P03355_PLV919 |
| GSSEAAAK | 12,596 | PERV_Q4VFZ2_3mut |
| GGGG | 12,597 | MLVMS_P03355_3mutA_WS |
| GGGGGS | 12,598 | MLVMS_P03355_3mut |
| GGGGSSEAAAK | 12,599 | PERV_Q4VFZ2_3mut |
| EAAAKEAAAKEAAAKEAAAKEAAAKEAAAK | 12,600 | SFV3L_P27401-Pro_2mutA |
| GGSEAAAKGSS | 12,601 | MLVMS_P03355_3mutA_WS |
| PAPGSSGGS | 12,602 | XMRV6_A1Z651_3mutA |
| GGSPAP | 12,603 | MLVMS_P03355_3mutA_WS |
| GGGGSSEAAAK | 12,604 | BAEVM_P10272_3mut |
| GGSGGSGGSGGS | 12,605 | AVIRE_P03360_3mutA |
| PAPGSSGGS | 12,606 | MLVFF_P26809_3mutA |
| GSSPAPGGG | 12,607 | MLVMS_P03355_3mutA_WS |
| GGGGGGG | 12,608 | MLVMS_P03355_3mutA_WS |
| EAAAKGGGGGS | 12,609 | MLVMS_P03355_3mutA_WS |
| EAAAKGGSGGG | 12,610 | MLVMS_P03355_PLV919 |
| GGGGSSEAAAK | 12,611 | XMRV6_A1Z651_3mutA |
| GGGGSEAAAKGGGGS | 12,612 | MLVBM_Q7SVK7_3mutA_WS |
| GSSGSS | 12,613 | MLVMS_P03355_PLV919 |
| GGSGGG | 12,614 | MLVMS_P03355_PLV919 |
| PAPEAAAKGGG | 12,615 | AVIRE_P03360_3mutA |
| AEAAAKEAAAKEAAAKEAAAKALEAEAAAKEAAAKEAAAKEAAAKA | 12,616 | FOAMV_P14350-Pro_2mutA |
| GGGGGSGSS | 12,617 | PERV_Q4VFZ2_3mut |
| GSSGSSGSSGSSGSS | 12,618 | KORV_Q9TTC1-Pro_3mut |
| GGGGSEAAAKGGGGS | 12,619 | MLVMS_P03355_3mutA_WS |
| GGGGGSPAP | 12,620 | FLV_P10273_3mut |
| GGGEAAAK | 12,621 | MLVMS_P03355_3mutA_WS |
| GGSGGSGGSGGS | 12,622 | FLV_P10273_3mutA |
| GGG | | MLVMS_P03355_PLV919 |
| GGSPAPEAAAK | 12,624 | BAEVM_P10272_3mutA |
| EAAAKEAAAK | 12,625 | FLV_P10273_3mutA |
| GGGEAAAKPAP | 12,626 | BAEVM_P10272_3mutA |
| GGGEAAAKGGS | 12,627 | PERV_Q4VFZ2_3mut |
| GGSGGSGGS | 12,628 | PERV_Q4VFZ2_3mut |
| EAAAKGGGPAP | 12,629 | XMRV6_A1Z651_3mutA |
| EAAAK | 12,630 | MLVBM_Q7SVK7_3mutA_WS |
| PAPEAAAKGGG | 12,631 | PERV_Q4VFZ2_3mut |
| EAAAKGSS | 12,632 | MLVCB_P08361_3mutA |
| GGSEAAAKGGG | 12,633 | MLVBM_Q7SVK7_3mutA_WS |
| GGGGSGGGGSGGGGSGGGGS | 12,634 | XMRV6_A1Z651_3mutA |
| GGGGSGGGGSGGGGSGGGGSGGGGS | 12,635 | BAEVM_P10272_3mut |
| GGGGSSPAP | 12,636 | PERV_Q4VFZ2_3mutA_WS |
| GGSGGSGGSGGSGGSGGS | 12,637 | PERV_Q4VFZ2_3mut |
| GGGEAAAKPAP | 12,638 | PERV_Q4VFZ2_3mut |
| EAAAKEAAAK | 12,639 | BAEVM_P10272_3mutA |
| GGSGSSEAAAK | 12,640 | XMRV6_A1Z651_3mutA |
| PAPEAAAKGSS | 12,641 | WMSV_P03359_3mutA |
| PAPAPAPAPAP | 12,642 | XMRV6_A1Z651_3mutA |
| GSSGGGEAAAK | 12,643 | MLVMS_P03355_PLV919 |
| GSSPAPGGG | 12,644 | MLVFF_P26809_3mutA |
| GGSPAPEAAAK | 12,645 | MLVFF_P26809_3mut |
| PAPGGSEAAAK | 12,646 | PERV_Q4VFZ2_3mut |
| GGGGSS | 12,647 | MLVFF_P26809_3mutA |
| GGSGSSGGG | 12,648 | BAEVM_P10272_3mutA |
| GSSGGGEAAAK | 12,649 | MLVMS_P03355_3mutA_WS |
| EAAAKGGS | 12,650 | MLVBM_Q7SVK7_3mutA_WS |
| GGGPAPGGS | 12,651 | MLVMS_P03355_PLV919 |
| EAAAKEAAAK | 12,652 | MLVMS_P03355_PLV919 |
| GSSGSSGSS | 12,653 | MLVMS_P03355_PLV919 |
| GGGEAAAKPAP | 12,654 | MLVAV_P03356_3mutA |
| SGSETPGTSESATPES | 12,655 | FLV_P10273_3mutA |
| PAPAPAPAPAP | 12,656 | KORV_Q9TTC1-Pro_3mut |
| AEAAAKEAAAKEAAAKEAAAKALEAEAAAKEAAAKEAAAKEAAAKA | 12,657 | BAEVM_P10272_3mutA |
| PAPGSSGGG | 12,658 | MLVMS_P03355_3mutA_WS |
| GSSGGGEAAAK | 12,659 | XMRV6_A1Z651_3mutA |
| GGGGSGGGGSGGGGSGGGGSGGGGS | 12,660 | XMRV6_A1Z651_3mutA |
| GGGGSSPAP | 12,661 | MLVFF_P26809_3mutA |
| GGSGGGPAP | 12,662 | PERV_Q4VFZ2_3mutA_WS |
| GSS | | PERV_Q4VFZ2_3mut |
| EAAAKGSSPAP | 12,664 | MLVMS_P03355_3mut |
| EAAAKGGG | 12,665 | XMRV6_A1Z651_3mutA |
| GSSGSSGSSGSS | 12,666 | WMSV_P03359_3mutA |
| PAPEAAAKGSS | 12,667 | MLVMS_P03355_PLV919 |
| GSSEAAAK | 12,668 | AVIRE_P03360_3mutA |
| EAAAKGGSGSS | 12,669 | AVIRE_P03360_3mutA |
| GSSEAAAK | 12,670 | MLVMS_P03355_3mut |
| GGSGSSEAAAK | 12,671 | MLVMS_P03355_PLV919 |
| GGSEAAAKGGG | 12,672 | MLVFF_P26809_3mutA |
| GGGGSGGGGSGGGGSGGGGS | 12,673 | MLVAV_P03356_3mutA |
| PAPAPAPAPAPAP | 12,674 | MLVFF_P26809_3mut |
| EAAAKPAPGSS | 12,675 | KORV_Q9TTC1-Pro_3mut |
| PAPGSSEAAAK | 12,676 | MLVAV_P03356_3mutA |
| GGGGSSPAP | 12,677 | WMSV_P03359_3mutA |
| EAAAKGGGGGS | 12,678 | MLVMS_P03355_3mutA_WS |
| GGGEAAAKGGS | 12,679 | MLVMS_P03355_3mut |
| GGSGSSGGG | 12,680 | MLVMS_P03355_3mut |
| GGGPAPGGS | 12,681 | MLVAV_P03356_3mutA |
| PAPGGGGGS | 12,682 | MLVMS_P03355_PLV919 |
| GGGPAPGSS | 12,683 | PERV_Q4VFZ2_3mut |
| GGGGGGG | 12,684 | MLVFF_P26809_3mutA |
| GGSGGGGSS | 12,685 | MLVCB_P08361_3mutA |
| GGGGGG | 12,686 | FLV_P10273_3mutA |
| GGSEAAAKGSS | 12,687 | PERV_Q4VFZ2_3mut |
| GGSPAPGGG | 12,688 | BAEVM_P10272_3mutA |
| GGSPAPGSS | 12,689 | AVIRE_P03360_3mutA |
| GGSGGSGGSGGS | 12,690 | KORV_Q9TTC1_3mut |
| EAAAKEAAAKEAAAKEAAAKEAAAK | 12,691 | MLVBM_Q7SVK7_3mut |
| PAPGSSGGS | 12,692 | XMRV6_A1Z651_3mut |
| EAAAKGGGGSS | 12,693 | PERV_Q4VFZ2_3mutA_WS |
| GGSGGSGGSGGSGGS | 12,694 | PERV_Q4VFZ2_3mutA_WS |
| PAPGGSGGG | 12,695 | MLVMS_P03355_PLV919 |
| PAPGSSGGG | 12,696 | PERV_Q4VFZ2_3mutA_WS |
| GSSGSS | 12,697 | BAEVM_P10272_3mutA |
| EAAAKGSS | 12,698 | MLVFF_P26809_3mutA |
| GGGPAP | 12,699 | MLVMS_P03355_PLV919 |
| EAAAKGGGGGS | 12,700 | MLVFF_P26809_3mutA |
| EAAAKGGSPAP | 12,701 | MLVBM_Q7SVK7_3mutA_WS |
| EAAAKEAAAKEAAAKEAAAKEAAAKEAAAK | 12,702 | WMSV_P03359_3mutA |
| GSSPAPGGG | 12,703 | MLVBM_Q7SVK7_3mutA_WS |
| GGGEAAAKGSS | 12,704 | AVIRE_P03360_3mutA |
| GGGGSSEAAAK | 12,705 | AVIRE_P03360_3mutA |
| GGGGGGGG | 12,706 | PERV_Q4VFZ2_3mutA_WS |
| PAPGSSEAAAK | 12,707 | BAEVM_P10272_3mutA |
| EAAAKGSS | 12,708 | MLVFF_P26809_3mut |
| GSSEAAAKGGG | 12,709 | MLVCB_P08361_3mutA |
| GGSEAAAK | 12,710 | MLVBM_Q7SVK7_3mutA_WS |
| GSSEAAAKGGG | 12,711 | PERV_Q4VFZ2_3mutA_WS |
| PAPGGSGGG | 12,712 | WMSV_P03359_3mutA |
| GSSGGSGGG | 12,713 | MLVCB_P08361_3mutA |
| EAAAKGSSGGG | 12,714 | FLV_P10273_3mutA |
| GSSEAAAK | 12,715 | MLVCB_P08361_3mutA |
| GSSGGGEAAAK | 12,716 | MLVMS_P03355_3mut |
| GGGGSGGGGS | 12,717 | MLVCB_P08361_3mutA |
| EAAAKGGGGSEAAAK | 12,718 | MLVBM_Q7SVK7_3mutA_WS |
| EAAAKGGG | 12,719 | PERV_Q4VFZ2_3mutA_WS |
| EAAAKGGSPAP | 12,720 | MLVMS_P03355_PLV919 |
| GGGPAPGGS | 12,721 | AVIRE_P03360_3mutA |
| GSSEAAAK | 12,722 | MLVBM_Q7SVK7_3mutA_WS |
| GSSGGGEAAAK | 12,723 | PERV_Q4VFZ2_3mut |
| SGSETPGTSESATPES | 12,724 | MLVMS_P03355_PLV919 |
| GGSGSSPAP | 12,725 | MLVMS_P03355_3mut |
| GGGGGG | 12,726 | MLVBM_Q7SVK7_3mutA_WS |
| GGSPAPGGG | 12,727 | XMRV6_A1Z651_3mutA |
| GGSGSS | 12,728 | PERV_Q4VFZ2_3mutA_WS |
| PAP | | MLVBM_Q7SVK7_3mutA_WS |
| EAAAKPAPGSS | 12,730 | MLVMS_P03355_PLV919 |
| EAAAKGGG | 12,731 | MLVMS_P03355_3mut |
| GSSEAAAKPAP | 12,732 | PERV_Q4VFZ2_3mutA_WS |
| GGGGSS | 12,733 | MLVMS_P03355_3mutA_WS |
| GGSGSSEAAAK | 12,734 | PERV_Q4VFZ2_3mut |
| GGGGSS | 12,735 | BAEVM_P10272_3mutA |
| PAPAP | 12,736 | MLVFF_P26809_3mut |
| PAPEAAAKGGG | 12,737 | BAEVM_P10272_3mutA |
| EAAAKGGS | 12,738 | MLVMS_P03355_PLV919 |
| PAPAPAPAPAPAP | 12,739 | PERV_Q4VFZ2_3mutA_WS |
| GGGGGSEAAAK | 12,740 | MLVMS_P03355_3mut |
| PAPGGS | 12,741 | PERV_Q4VFZ2_3mut |
| GGGGSS | 12,742 | MLVCB_P08361_3mutA |
| GGGGS | 12,743 | MLVAV_P03356_3mutA |
| GSSPAPEAAAK | 12,744 | MLVMS_P03355_PLV919 |
| GGGGSSGGS | 12,745 | MLVFF_P26809_3mutA |
| PAPEAAAKGSS | 12,746 | MLVMS_P03355_PLV919 |
| GGSGSSEAAAK | 12,747 | MLVMS_P03355_3mutA_WS |
| EAAAKGGG | 12,748 | MLVAV_P03356_3mutA |
| PAPGSSEAAAK | 12,749 | FLV_P10273_3mutA |
| EAAAKGSSGGG | 12,750 | MLVCB_P08361_3mutA |
| PAPEAAAK | 12,751 | KORV_Q9TTC1-Pro_3mutA |
| GGSPAPEAAAK | 12,752 | KORV_Q9TTC1-Pro_3mut |
| GGSGGSGGSGGSGGSGGS | 12,753 | MLVAV_P03356_3mutA |
| GSSEAAAKPAP | 12,754 | MLVBM_Q7SVK7_3mutA_WS |
| AEAAAKEAAAKEAAAKEAAAKALEAEAAAKEAAAKEAAAKEAAAKA | 12,755 | KORV_Q9TTC1-Pro_3mutA |
| GSSGGGEAAAK | 12,756 | XMRV6_A1Z651_3mut |
| PAPGGSGGG | 12,757 | AVIRE_P03360_3mutA |
| PAPGGSEAAAK | 12,758 | PERV_Q4VFZ2_3mutA_WS |
| GGGGS | 12,759 | MLVMS_P03355_3mutA_WS |
| GGGGSGGGGSGGGGS | 12,760 | MLVBM_Q7SVK7_3mutA_WS |
| PAPAPAPAPAP | 12,761 | PERV_Q4VFZ2_3mutA_WS |
| EAAAKEAAAKEAAAKEAAAKEAAAK | 12,762 | MLVMS_P03355_3mut |
| GSSGGSEAAAK | 12,763 | MLVMS_P03355_3mutA_WS |
| GGSGGSGGSGGS | 12,764 | WMSV_P03359_3mutA |
| EAAAKGSSGGG | 12,765 | WMSV_P03359_3mutA |
| EAAAKGGG | 12,766 | PERV_Q4VFZ2_3mutA_WS |
| SGSETPGTSESATPES | 12,767 | PERV_Q4VFZ2_3mut |
| PAPGSSGGS | 12,768 | MLVMS_P03355_3mutA_WS |
| PAPEAAAKGSS | 12,769 | PERV_Q4VFZ2_3mut |
| PAPEAAAK | 12,770 | AVIRE_P03360_3mutA |
| GSSEAAAKGGG | 12,771 | BAEVM_P10272_3mutA |
| GSSPAP | 12,772 | MLVAV_P03356_3mutA |
| EAAAKEAAAKEAAAKEAAAK | 12,773 | MLVFF_P26809_3mut |
| PAPGGSGSS | 12,774 | MLVAV_P03356_3mutA |
| GGGGSGGGGSGGGGS | 12,775 | PERV_Q4VFZ2_3mutA_WS |
| GSSGGSEAAAK | 12,776 | MLVCB_P08361_3mutA |
| EAAAKGGS | 12,777 | KORV_Q9TTC1-Pro_3mutA |
| EAAAKGGS | 12,778 | MLVFF_P26809_3mutA |
| GGSPAP | 12,779 | MLVMS_P03355_PLV919 |
| GGSGSS | 12,780 | MLVMS_P03355_PLV919 |
| SGSETPGTSESATPES | 12,781 | WMSV_P03359_3mut |
| GGGGGGG | 12,782 | WMSV_P03359_3mut |
| GGSPAPGSS | 12,783 | MLVCB_P08361_3mutA |
| GGGGSSGGS | 12,784 | WMSV_P03359_3mut |
| PAPGGS | 12,785 | MLVMS_P03355_PLV919 |
| PAPGSSGGS | 12,786 | MLVCB_P08361_3mutA |
| EAAAKEAAAKEAAAKEAAAKEAAAK | 12,787 | MLVFF_P26809_3mut |
| SGGSSGGSSGSETPGTSESATPESSGGSSGGSS | 12,788 | PERV_Q4VFZ2_3mut |
| GGSGGSGGSGGSGGS | 12,789 | BAEVM_P10272_3mutA |
| GSSEAAAK | 12,790 | PERV_Q4VFZ2_3mut |
| EAAAKEAAAKEAAAKEAAAK | 12,791 | KORV_Q9TTC1-Pro_3mutA |
| GGSGGSGGSGGSGGS | 12,792 | MLVMS_P03355_3mut |
| PAPAPAPAPAPAP | 12,793 | MLVMS_P03355_3mut |
| GGSPAPEAAAK | 12,794 | MLVMS_P03355_PLV919 |
| EAAAK | 12,795 | WMSV_P03359_3mutA |
| EAAAKGSSGGS | 12,796 | MLVBM_Q7SVK7_3mutA_WS |
| GGSGGGGSS | 12,797 | MLVMS_P03355_3mutA_WS |
| GGGEAAAKPAP | 12,798 | MLVMS_P03355_3mut |
| EAAAKGGSGGG | 12,799 | XMRV6_A1Z651_3mutA |
| GGGGGSEAAAK | 12,800 | KORV_Q9TTC1-Pro_3mutA |
| GGGGGG | 12,801 | BAEVM_P10272_3mutA |
| GGGGGG | 12,802 | MLVMS_P03355_3mut |
| GGGGGGG | 12,803 | MLVBM_Q7SVK7_3mutA_WS |
| AEAAAKEAAAKEAAAKEAAAKALEAEAAAKEAAAKEAAAKEAAAKA | 12,804 | AVIRE_P03360 |
| PAPGSSGGS | 12,805 | PERV_Q4VFZ2_3mut |
| GGGGGS | 12,806 | XMRV6_A1Z651_3mut |
| EAAAKPAP | 12,807 | XMRV6_A1Z651_3mutA |
| GGG | | MLVMS_P03355_3mutA_WS |
| AEAAAKEAAAKEAAAKEAAAKALEAEAAAKEAAAKEAAAKEAAAKA | 12,809 | FLV_P10273_3mut |
| EAAAKGSSPAP | 12,810 | MLVMS_P03355_3mut |
| SGSETPGTSESATPES | 12,811 | BAEVM_P10272_3mutA |
| GGSPAPEAAAK | 12,812 | MLVMS_P03355_3mut |
| GSSGSSGSSGSS | 12,813 | MLVAV_P03356_3mutA |
| AEAAAKEAAAKEAAAKEAAAKALEAEAAAKEAAAKEAAAKEAAAKA | 12,814 | MLVMS_P03355_3mut |
| GGSPAP | 12,815 | MLVCB_P08361_3mutA |
| GGGGGSEAAAK | 12,816 | MLVMS_P03355_3mutA_WS |
| GGGGG | 12,817 | MLVFF_P26809_3mutA |
| GSSEAAAK | 12,818 | MLVAV_P03356_3mutA |
| GGS | | BAEVM_P10272_3mut |
| EAAAKGGSPAP | 12,820 | MLVCB_P08361_3mutA |
| PAPAPAPAP | 12,821 | FLV_P10273_3mutA |
| PAPGGGEAAAK | 12,822 | MLVCB_P08361_3mutA |
| GGGGSSEAAAK | 12,823 | MLVMS_P03355_3mutA_WS |
| GGGGG | 12,824 | PERV_Q4VFZ2_3mutA_WS |
| GGSGGSGGSGGSGGSGGS | 12,825 | PERV_Q4VFZ2_3mut |
| GGGGG | 12,826 | MLVMS_P03355_3mut |
| PAPEAAAKGGG | 12,827 | MLVBM_Q7SVK7_3mutA_WS |
| GSSGGGPAP | 12,828 | XMRV6_A1Z651_3mutA |
| GSSGSSGSSGSSGSSGSS | 12,829 | PERV_Q4VFZ2_3mutA_WS |
| EAAAKGGSPAP | 12,830 | PERV_Q4VFZ2_3mut |
| GSSGGSEAAAK | 12,831 | MLVMS_P03355_PLV919 |
| GSS | | PERV_Q4VFZ2_3mut |
| EAAAKGGS | 12,833 | WMSV_P03359_3mutA |
| GGGGGSPAP | 12,834 | PERV_Q4VFZ2_3mutA_WS |
| EAAAKGSS | 12,835 | MLVMS_P03355_PLV919 |
| EAAAKGGGGSS | 12,836 | KORV_Q9TTC1-Pro_3mutA |
| PAPGSSGGG | 12,837 | PERV_Q4VFZ2_3mut |
| GGGGSSEAAAK | 12,838 | MLVFF_P26809_3mut |
| PAPAPAP | 12,839 | MLVMS_P03355_3mut |
| GSSGGSEAAAK | 12,840 | XMRV6_A1Z651_3mut |
| PAPEAAAKGSS | 12,841 | MLVMS_P03355_3mutA_WS |
| GGSGGSGGSGGSGGS | 12,842 | MLVMS_P03355_3mutA_WS |
| GGSGSSPAP | 12,843 | XMRV6_A1Z651_3mutA |
| GGGGSSPAP | 12,844 | MLVMS_P03355_PLV919 |
| GGGGS | 12,845 | MLVCB_P08361_3mutA |
| EAAAKEAAAKEAAAKEAAAK | 12,846 | PERV_Q4VFZ2_3mutA_WS |
| EAAAKEAAAK | 12,847 | KORV_Q9TTC1_3mutA |
| PAPGGGEAAAK | 12,848 | BAEVM_P10272_3mutA |
| GSSGGSEAAAK | 12,849 | XMRV6_A1Z651_3mutA |
| EAAAKEAAAKEAAAKEAAAKEAAAKEAAAK | 12,850 | FLV_P10273_3mut |
| GSSEAAAKPAP | 12,851 | MLVMS_P03355_3mutA_WS |
| EAAAKPAPGSS | 12,852 | PERV_Q4VFZ2_3mutA_WS |
| GSSGGSPAP | 12,853 | XMRV6_A1Z651_3mutA |
| GSSEAAAKGGG | 12,854 | PERV_Q4VFZ2_3mut |
| GGGEAAAKGGS | 12,855 | WMSV_P03359_3mutA |
| GSSEAAAKGGG | 12,856 | MLVFF_P26809_3mut |
| PAPAPAP | 12,857 | KORV_Q9TTC1-Pro_3mutA |
| EAAAKGGSPAP | 12,858 | MLVMS_P03355_3mutA_WS |
| PAPGGSEAAAK | 12,859 | PERV_Q4VFZ2_3mut |
| GGGGS | 12,860 | MLVBM_Q7SVK7_3mutA_WS |
| EAAAKGSSGGG | 12,861 | KORV_Q9TTC1_3mut |
| EAAAKGGGPAP | 12,862 | MLVCB_P08361_3mutA |
| EAAAKGSS | 12,863 | BAEVM_P10272_3mutA |
| GGSPAPGGG | 12,864 | MLVBM_Q7SVK7_3mutA_WS |
| GGGGSEAAAKGGGGS | 12,865 | MLVMS_P03355_3mutA_WS |
| GGGEAAAKGGS | 12,866 | PERV_Q4VFZ2_3mutA_WS |
| EAAAKGGGGSS | 12,867 | MLVMS_P03355_3mutA_WS |
| EAAAKGGGPAP | 12,868 | MLVFF_P26809_3mut |
| GSSPAP | 12,869 | PERV_Q4VFZ2_3mutA_WS |
| EAAAKGGS | 12,870 | MLVMS_P03355_3mut |
| GGGGSS | 12,871 | KORV_Q9TTC1-Pro_3mutA |
| EAAAKGSSPAP | 12,872 | MLVMS_P03355_3mutA_WS |
| GGGPAP | 12,873 | PERV_Q4VFZ2_3mut |
| EAAAKGSSGGS | 12,874 | XMRV6_A1Z651_3mutA |
| PAPGGG | 12,875 | MLVAV_P03356_3mutA |
| GSSPAPEAAAK | 12,876 | BAEVM_P10272_3mutA |
| GGGPAP | 12,877 | MLVBM_Q7SVK7_3mutA_WS |
| GSSGGGGGS | 12,878 | AVIRE_P03360_3mutA |
| SGSETPGTSESATPES | 12,879 | MLVMS_P03355_PLV919 |
| GGGPAP | 12,880 | MLVFF_P26809_3mut |
| EAAAKGGGGSS | 12,881 | XMRV6_A1Z651_3mutA |
| GGGGSSPAP | 12,882 | XMRV6_A1Z651_3mut |
| GGGGSEAAAKGGGGS | 12,883 | MLVMS_P03355_3mut |
| GSSPAP | 12,884 | MLVBM_Q7SVK7_3mutA_WS |
| GGSGSSEAAAK | 12,885 | FLV_P10273_3mutA |
| SGSETPGTSESATPES | 12,886 | MLVBM_Q7SVK7_3mutA_WS |
| PAPGGG | 12,887 | AVIRE_P03360_3mutA |
| GGGEAAAKPAP | 12,888 | MLVMS_P03355_3mutA_WS |
| EAAAKGGSGSS | 12,889 | PERV_Q4VFZ2_3mut |
| GGSPAPGGG | 12,890 | MLVAV_P03356_3mutA |
| PAPGGSGSS | 12,891 | BAEVM_P10272_3mutA |
| GSSGGSPAP | 12,892 | MLVFF_P26809_3mutA |
| EAAAKGSSGGG | 12,893 | PERV_Q4VFZ2_3mut |
| GGGGSGGGGS | 12,894 | PERV_Q4VFZ2_3mutA_WS |
| GSSGGGGGS | 12,895 | BAEVM_P10272_3mutA |
| GGGGSSGGS | 12,896 | MLVBM_Q7SVK7_3mutA_WS |
| EAAAKGGS | 12,897 | PERV_Q4VFZ2_3mutA_WS |
| GSSGSSGSSGSS | 12,898 | MLVMS_P03355_3mut |
| GGS | | MLVMS_P03355_3mutA_WS |
| GSSGGSEAAAK | 12,900 | MLVBM_Q7SVK7_3mutA_WS |
| SGGSSGGSSGSETPGTSESATPESSGGSSGGSS | 12,901 | XMRV6_A1Z651 |
| GGGGG | 12,902 | FLV_P10273_3mutA |
| PAPEAAAKGSS | 12,903 | PERV_Q4VFZ2_3mut |
| GGGGGG | 12,904 | WMSV_P03359_3mut |
| EAAAKGGG | 12,905 | BAEVM_P10272_3mutA |
| GGGGSS | 12,906 | MLVMS_P03355_3mutA_WS |
| GSSGGGEAAAK | 12,907 | KORV_Q9TTC1_3mut |
| GGSGSS | 12,908 | AVIRE_P03360_3mutA |
| EAAAKPAP | 12,909 | MLVMS_P03355_3mut |
| EAAAKEAAAKEAAAK | 12,910 | FLV_P10273_3mutA |
| GGGG | 12,911 | XMRV6_A1Z651_3mutA |
| GSSPAPGGS | 12,912 | BAEVM_P10272_3mutA |
| GSSGGGGGS | 12,913 | MLVFF_P26809_3mutA |
| GGGGSSGGS | 12,914 | MLVAV_P03356_3mutA |
| GGS | | PERV_Q4VFZ2_3mut |
| GGGGG | 12,916 | WMSV_P03359_3mutA |
| GSSGSSGSSGSSGSSGSS | 12,917 | FLV_P10273_3mutA |
| PAPGGGGSS | 12,918 | MLVAV_P03356_3mutA |
| GGGGGGGG | 12,919 | BAEVM_P10272_3mutA |
| SGSETPGTSESATPES | 12,920 | MLVCB_P08361_3mutA |
| PAPGGG | 12,921 | BAEVM_P10272_3mutA |
| GSSGSSGSS | 12,922 | MLVCB_P08361_3mutA |
| GGSGSS | 12,923 | MLVMS_P03355_3mutA_WS |
| EAAAKGGGGSEAAAK | 12,924 | WMSV_P03359_3mutA |
| GGGGGGGG | 12,925 | FLV_P10273_3mutA |
| GSSGSS | 12,926 | MLVMS_P03355_3mutA_WS |
| PAPEAAAKGGS | 12,927 | XMRV6_A1Z651_3mutA |
| EAAAKEAAAK | 12,928 | MLVMS_P03355_3mut |
| GGGGSGGGGSGGGGS | 12,929 | BAEVM_P10272_3mutA |
| EAAAKGSSPAP | 12,930 | MLVMS_P03355_PLV919 |
| GGGGSSEAAAK | 12,931 | MLVMS_P03355_3mut |
| GGGGSSEAAAK | 12,932 | BAEVM_P10272_3mutA |
| PAPGGSGSS | 12,933 | PERV_Q4VFZ2_3mut |
| GGSGGGEAAAK | 12,934 | MLVFF_P26809_3mut |
| PAPEAAAKGGS | 12,935 | PERV_Q4VFZ2_3mut |
| GGGPAPGSS | 12,936 | AVIRE_P03360_3mut |
| PAPGGSGGG | 12,937 | PERV_Q4VFZ2_3mutA_WS |
| GGGGGGGG | 12,938 | PERV_Q4VFZ2_3mutA_WS |
| GSSEAAAK | 12,939 | MLVMS_P03355_3mutA_WS |
| GGGGSGGGGSGGGGS | 12,940 | PERV_Q4VFZ2_3mutA_WS |
| EAAAKGGS | 12,941 | MLVMS_P03355_3mut |
| GGGGGSGSS | 12,942 | MLVCB_P08361_3mut |
| GGGPAP | 12,943 | KORV_Q9TTC1-Pro_3mutA |
| EAAAKPAPGGG | 12,944 | MLVCB_P08361_3mut |
| GSSGGSPAP | 12,945 | MLVCB_P08361_3mutA |
| SGGSSGGSSGSETPGTSESATPESSGGSSGGSS | 12,946 | MLVMS_P03355_3mut |
| PAPAPAPAP | 12,947 | MLVMS_P03355_3mut |
| GSSGGS | 12,948 | XMRV6_A1Z651_3mutA |
| GSSEAAAKGGG | 12,949 | MLVMS_P03355_3mut |
| GGSGSSPAP | 12,950 | MLVMS_P03355_3mutA_WS |
| GSSEAAAKGGS | 12,951 | MLVMS_P03355_PLV919 |
| EAAAKEAAAKEAAAKEAAAKEAAAK | 12,952 | BAEVM_P10272_3mut |
| PAPGGGGSS | 12,953 | KORV_Q9TTC1_3mutA |
| EAAAKGSS | 12,954 | MLVMS_P03355_3mutA_WS |
| AEAAAKEAAAKEAAAKEAAAKALEAEAAAKEAAAKEAAAKEAAAKA | 12,955 | FFV_O93209_2mut |
| GGSGGSGGSGGSGGSGGS | 12,956 | BAEVM_P10272_3mutA |
| GGGGGG | 12,957 | MLVMS_P03355_PLV919 |
| PAPEAAAK | 12,958 | BAEVM_P10272_3mutA |
| GGSGSSEAAAK | 12,959 | MLVAV_P03356_3mutA |
| GGG | | MLVCB_P08361_3mutA |
| GGGGG | 12,961 | MLVCB_P08361_3mutA |
| GGSGGSGGSGGS | 12,962 | KORV_Q9TTC1-Pro_3mutA |
| GSSGSSGSSGSSGSSGSS | 12,963 | XMRV6_A1Z651_3mutA |
| GSSEAAAKPAP | 12,964 | FLV_P10273_3mutA |
| GGGEAAAKPAP | 12,965 | MLVCB_P08361_3mutA |
| GSSGSSGSS | 12,966 | MLVMS_P03355_3mutA_WS |
| PAPAPAPAP | 12,967 | MLVMS_P03355_PLV919 |
| EAAAKGGG | 12,968 | MLVMS_P03355_PLV919 |
| PAPAPAPAPAPAP | 12,969 | FLV_P10273_3mutA |
| EAAAKGGSGSS | 12,970 | MLVMS_P03355_3mut |
| GGGGGG | 12,971 | PERV_Q4VFZ2_3mutA_WS |
| PAPGGG | 12,972 | MLVCB_P08361_3mutA |
| GGGGGSGSS | 12,973 | KORV_Q9TTC1_3mutA |
| GGGGSGGGGSGGGGSGGGGS | 12,974 | XMRV6_A1Z651_3mut |
| GGSGGSGGS | 12,975 | KORV_Q9TTC1-Pro_3mutA |
| EAAAKPAPGGG | 12,976 | MLVMS_P03355_3mutA_WS |
| AEAAAKEAAAKEAAAKEAAAKALEAEAAAKEAAAKEAAAKEAAAKA | 12,977 | XMRV6_A1Z651 |
| GGGGSGGGGSGGGGSGGGGSGGGGSGGGGS | 12,978 | FLV_P10273_3mutA |
| EAAAKGGGGSEAAAK | 12,979 | PERV_Q4VFZ2_3mutA_WS |
| GGGPAPGSS | 12,980 | AVIRE_P03360_3mutA |
| GGGGG | 12,981 | MLVMS_P03355_3mutA_WS |
| GGGGSGGGGSGGGGSGGGGSGGGGSGGGGS | 12,982 | MLVMS_P03355_3mut |
| GGGGSGGGGS | 12,983 | MLVMS_P03355_3mutA_WS |
| EAAAKGGSPAP | 12,984 | XMRV6_A1Z651_3mutA |
| EAAAKGSSPAP | 12,985 | AVIRE_P03360_3mutA |
| PAPGGSGSS | 12,986 | KORV_Q9TTC1-Pro_3mutA |
| GSS | | MLVBM_Q7SVK7_3mutA_WS |
| GSS | | WMSV_P03359_3mut |
| GGGPAPGSS | 12,989 | MLVFF_P26809_3mutA |
| EAAAKPAP | 12,990 | MLVMS_P03355_3mut |
| GSSPAPEAAAK | 12,991 | FLV_P10273_3mutA |
| GGSPAPGSS | 12,992 | MLVBM_Q7SVK7_3mutA_WS |
| GGGGGSEAAAK | 12,993 | XMRV6_A1Z651_3mut |
| PAPEAAAKGGG | 12,994 | WMSV_P03359_3mutA |
| PAPGGG | 12,995 | PERV_Q4VFZ2_3mut |
| GGSPAPEAAAK | 12,996 | WMSV_P03359_3mutA |
| GGSGGGGSS | 12,997 | PERV_Q4VFZ2_3mut |
| EAAAKGGGGSS | 12,998 | PERV_Q4VFZ2_3mut |
| EAAAKGGSPAP | 12,999 | AVIRE_P03360_3mut |
| GGSGGGGSS | 13,000 | WMSV_P03359_3mutA |
| PAPGSSEAAAK | 13,001 | MLVFF_P26809_3mut |
| GSSEAAAK | 13,002 | MLVMS_P03355_PLV919 |
| GSAGSAAGSGEF | 13,003 | AVIRE_P03360_3mutA |
| EAAAKGGSGSS | 13,004 | MLVMS_P03355_3mut |
| GGSEAAAKPAP | 13,005 | MLVMS_P03355_PLV919 |
| GGGGSGGGGSGGGGSGGGGSGGGGS | 13,006 | MLVFF_P26809_3mutA |
| PAPGSSEAAAK | 13,007 | PERV_Q4VFZ2_3mutA_WS |
| GGGGSSPAP | 13,008 | MLVMS_P03355_3mutA_WS |
| PAPAPAP | 13,009 | MLVCB_P08361_3mutA |
| EAAAKPAPGGG | 13,010 | MLVBM_Q7SVK7_3mutA_WS |
| GGGPAPGSS | 13,011 | BAEVM_P10272_3mutA |
| PAP | | MLVMS_P03355_3mutA_WS |
| PAPGGSGGG | 13,013 | MLVMS_P03355_3mutA_WS |
| GGSGGSGGSGGSGGS | 13,014 | MLVBM_Q7SVK7_3mutA_WS |
| PAPAPAPAP | 13,015 | XMRV6_A1Z651_3mut |
| GSSPAPGGG | 13,016 | MLVMS_P03355_3mutA_WS |
| GSSPAPGGG | 13,017 | MLVMS_P03355_3mut |
| PAPGGG | 13,018 | MLVMS_P03355_PLV919 |
| GGGEAAAKGSS | 13,019 | WMSV_P03359_3mut |
| EAAAKGSS | 13,020 | KORV_Q9TTC1-Pro_3mutA |
| EAAAKGGS | 13,021 | PERV_Q4VFZ2_3mut |
| EAAAKEAAAKEAAAKEAAAKEAAAK | 13,022 | PERV_Q4VFZ2_3mut |
| PAPEAAAKGGG | 13,023 | MLVMS_P03355_PLV919 |
| EAAAKGSSGGG | 13,024 | MLVFF_P26809_3mut |
| AEAAAKEAAAKEAAAKEAAAKALEAEAAAKEAAAKEAAAKEAAAKA | 13,025 | PERV_Q4VFZ2 |
| EAAAKEAAAKEAAAKEAAAKEAAAKEAAAK | 13,026 | MLVAV_P03356_3mutA |
| GSSGGSGGG | 13,027 | MLVFF_P26809_3mut |
| GSSGSSGSSGSS | 13,028 | PERV_Q4VFZ2_3mutA_WS |
| GGSPAPGGG | 13,029 | MLVMS_P03355_PLV919 |
| GSS | | BAEVM_P10272_3mut |
| GGGPAPGSS | 13,031 | MLVMS_P03355_3mutA_WS |
| GGGGSS | 13,032 | KORV_Q9TTC1_3mutA |
| GSSGGSGGG | 13,033 | BAEVM_P10272_3mutA |
| EAAAKEAAAKEAAAK | 13,034 | MLVCB_P08361_3mutA |
| SGGSSGGSSGSETPGTSESATPESSGGSSGGSS | 13,035 | FLV_P10273_3mutA |
| PAPGGGGGS | 13,036 | PERV_Q4VFZ2_3mut |
| PAPAPAPAPAP | 13,037 | KORV_Q9TTC1-Pro_3mutA |
| EAAAK | 13,038 | MLVMS_P03355_3mutA_WS |
| GGG | | MLVCB_P08361_3mut |
| GGSEAAAKGGG | 13,040 | BAEVM_P10272_3mutA |
| GGGGGSGSS | 13,041 | MLVAV_P03356_3mutA |
| EAAAKGSSPAP | 13,042 | MLVBM_Q7SVK7_3mutA_WS |
| GGSGGSGGS | 13,043 | XMRV6_A1Z651_3mut |
| EAAAKPAPGGG | 13,044 | KORV_Q9TTC1-Pro_3mutA |
| GGGPAPEAAAK | 13,045 | FLV_P10273_3mutA |
| GGSPAPEAAAK | 13,046 | MLVMS_P03355_3mutA_WS |
| GGSGGSGGSGGSGGS | 13,047 | MLVFF_P26809_3mut |
| EAAAKGGSGSS | 13,048 | MLVMS_P03355_PLV919 |
| GGGEAAAKGGS | 13,049 | MLVBM_Q7SVK7_3mutA_WS |
| PAPAPAPAP | 13,050 | BAEVM_P10272_3mutA |
| EAAAKEAAAKEAAAKEAAAK | 13,051 | MLVMS_P03355_3mut |
| EAAAKPAP | 13,052 | XMRV6_A1Z651_3mut |
| EAAAKEAAAK | 13,053 | MLVBM_Q7SVK7_3mutA_WS |
| EAAAKGGG | 13,054 | BAEVM_P10272_3mut |
| EAAAKGSS | 13,055 | MLVAV_P03356_3mutA |
| EAAAKEAAAKEAAAKEAAAKEAAAKEAAAK | 13,056 | MLVFF_P26809_3mut |
| GGGPAPGSS | 13,057 | PERV_Q4VFZ2_3mutA_WS |
| GGGG | 13,058 | PERV_Q4VFZ2_3mut |
| EAAAKGGSGSS | 13,059 | MLVMS_P03355_PLV919 |
| GGGGSGGGGSGGGGS | 13,060 | MLVMS_P03355_3mutA_WS |
| EAAAK | 13,061 | MLVMS_P03355_3mutA_WS |
| GGGGSS | 13,062 | PERV_Q4VFZ2 |
| PAPEAAAKGGS | 13,063 | MLVCB_P08361_3mut |
| GSS | | MLVMS_P03355_3mut |
| GSAGSAAGSGEF | 13,065 | MLVFF_P26809_3mutA |
| EAAAKEAAAKEAAAKEAAAKEAAAKEAAAK | 13,066 | KORV_Q9TTC1-Pro_3mut |
| GGGGSGGGGS | 13,067 | AVIRE_P03360_3mutA |
| EAAAK | 13,068 | MLVMS_P03355_3mut |
| GGGPAPGGS | 13,069 | PERV_Q4VFZ2_3mut |
| GGGGSGGGGSGGGGS | 13,070 | MLVMS_P03355_PLV919 |
| PAPGGG | 13,071 | MLVMS_P03355_3mutA_WS |
| GGGEAAAKPAP | 13,072 | PERV_Q4VFZ2_3mutA_WS |
| EAAAKPAPGSS | 13,073 | KORV_Q9TTC1-Pro_3mutA |
| PAPGSS | 13,074 | KORV_Q9TTC1_3mutA |
| GSAGSAAGSGEF | 13,075 | PERV_Q4VFZ2_3mut |
| PAPGGGGSS | 13,076 | KORV_Q9TTC1-Pro_3mutA |
| GSSGGGEAAAK | 13,077 | MLVCB_P08361_3mutA |
| GSS | | AVIRE_P03360_3mutA |
| GSSGSSGSSGSS | 13,079 | XMRV6_A1Z651_3mutA |
| PAPEAAAKGGG | 13,080 | MLVMS_P03355_PLV919 |
| GGGPAPEAAAK | 13,081 | MLVCB_P08361_3mutA |
| PAPGGGGGS | 13,082 | MLVCB_P08361_3mutA |
| EAAAKEAAAKEAAAKEAAAK | 13,083 | PERV_Q4VFZ2_3mutA_WS |
| GGGGGSPAP | 13,084 | MLVFF_P26809_3mutA |
| GSSGSSGSSGSSGSS | 13,085 | PERV_Q4VFZ2 |
| GSSPAPEAAAK | 13,086 | MLVMS_P03355_PLV919 |
| GSSGSSGSSGSSGSSGSS | 13,087 | MLVBM_Q7SVK7_3mutA_WS |
| GSSGSSGSSGSSGSSGSS | 13,088 | MLVMS_P03355_3mutA_WS |
| GGSPAPEAAAK | 13,089 | MLVAV_P03356_3mutA |
| GSSGGG | 13,090 | BAEVM_P10272_3mut |
| EAAAKGSSGGS | 13,091 | KORV_Q9TTC1-Pro_3mutA |
| GGSGSSEAAAK | 13,092 | MLVMS_P03355_3mutA_WS |
| GGGPAPEAAAK | 13,093 | MLVFF_P26809_3mutA |
| GGGPAPGGS | 13,094 | MLVMS_P03355_3mutA_WS |
| GGGGG | 13,095 | MLVMS_P03355_PLV919 |
| GGGEAAAKPAP | 13,096 | MLVBM_Q7SVK7_3mutA_WS |
| GGGGSGGGGS | 13,097 | WMSV_P03359_3mut |
| GGGPAPEAAAK | 13,098 | PERV_Q4VFZ2_3mut |
| GGSGSSEAAAK | 13,099 | MLVMS_P03355_PLV919 |
| EAAAKGGGPAP | 13,100 | MLVMS_P03355_3mutA_WS |
| GSSGSSGSSGSSGSS | 13,101 | KORV_Q9TTC1-Pro_3mutA |
| PAPAP | 13,102 | WMSV_P03359_3mutA |
| GGSPAPGSS | 13,103 | MLVAV_P03356_3mutA |
| GGSGGGPAP | 13,104 | MLVMS_P03355_3mut |
| GGSPAP | 13,105 | MLVMS_P03355_PLV919 |
| EAAAKGGSPAP | 13,106 | PERV_Q4VFZ2_3mut |
| GSSPAPGGG | 13,107 | KORV_Q9TTC1-Pro_3mutA |
| GSAGSAAGSGEF | 13,108 | MLVMS_P03355_3mut |
| GGSPAP | 13,109 | PERV_Q4VFZ2_3mut |
| GSSGSS | 13,110 | KORV_Q9TTC1-Pro_3mut |
| GGGPAPGSS | 13,111 | MLVMS_P03355_3mutA_WS |
| AEAAAKEAAAKEAAAKEAAAKALEAEAAAKEAAAKEAAAKEAAAKA | 13,112 | FOAMV_P14350 |
| PAPGSSGGG | 13,113 | MLVMS_P03355_PLV919 |
| GGSEAAAKPAP | 13,114 | BAEVM_P10272_3mutA |
| GGGGGS | 13,115 | MLVCB_P08361_3mutA |
| PAPEAAAKGGS | 13,116 | MLVMS_P03355_3mutA_WS |
| EAAAKEAAAKEAAAKEAAAKEAAAKEAAAK | 13,117 | BAEVM_P10272_3mutA |
| GGSEAAAK | 13,118 | BAEVM_P10272_3mutA |
| GSSPAPEAAAK | 13,119 | MLVMS_P03355_3mutA_WS |
| PAPGGG | 13,120 | WMSV_P03359_3mut |
| EAAAKPAP | 13,121 | PERV_Q4VFZ2_3mut |
| GSSGSSGSSGSSGSS | 13,122 | WMSV_P03359_3mut |
| PAPGGG | 13,123 | MLVBM_Q7SVK7_3mutA_WS |
| GGSGGGEAAAK | 13,124 | BAEVM_P10272_3mutA |
| PAPGGS | 13,125 | MLVMS_P03355_3mut |
| GGSGGSGGSGGS | 13,126 | MLVBM_Q7SVK7_3mutA_WS |
| EAAAKEAAAKEAAAKEAAAK | 13,127 | PERV_Q4VFZ2_3mut |
| GGSEAAAKGGG | 13,128 | WMSV_P03359_3mutA |
| GGGPAP | 13,129 | BAEVM_P10272_3mutA |
| GGGGSGGGGSGGGGSGGGGSGGGGSGGGGS | 13,130 | XMRV6_A1Z651_3mut |
| GGSPAPGSS | 13,131 | KORV_Q9TTC1_3mut |
| GGGPAPGSS | 13,132 | MLVMS_P03355_3mut |
| GGGGSSGGS | 13,133 | BAEVM_P10272_3mutA |
| GGGEAAAKGSS | 13,134 | KORV_Q9TTC1-Pro_3mutA |
| PAPAP | 13,135 | MLVBM_Q7SVK7_3mutA_WS |
| GGSPAPGGG | 13,136 | PERV_Q4VFZ2_3mut |
| PAPGSS | 13,137 | PERV_Q4VFZ2_3mutA_WS |
| GSSGGSPAP | 13,138 | MLVBM_Q7SVK7_3mutA_WS |
| EAAAKGGGGSEAAAK | 13,139 | PERV_Q4VFZ2_3mut |
| GSSEAAAKGGS | 13,140 | KORV_Q9TTC1-Pro_3mut |
| PAPAPAPAP | 13,141 | KORV_Q9TTC1-Pro_3mutA |
| GGSEAAAKPAP | 13,142 | WMSV_P03359_3mutA |
| PAPGGS | 13,143 | FLV_P10273_3mutA |
| EAAAKGGGPAP | 13,144 | PERV_Q4VFZ2_3mut |
| GGSGSSGGG | 13,145 | AVIRE_P03360_3mutA |
| EAAAKGGSGSS | 13,146 | BAEVM_P10272_3mutA |
| SGGSSGGSSGSETPGTSESATPESSGGSSGGSS | 13,147 | MLVCB_P08361_3mutA |
| GSSEAAAKGGS | 13,148 | XMRV6_A1Z651_3mutA |
| GGGGG | 13,149 | BAEVM_P10272_3mutA |
| GGGGSGGGGSGGGGSGGGGSGGGGSGGGGS | 13,150 | SFV3L_P27401_2mutA |
| GGGEAAAKGSS | 13,151 | MLVMS_P03355_PLV919 |
| EAAAKGGGGSEAAAK | 13,152 | KORV_Q9TTC1_3mutA |
| EAAAKGGG | 13,153 | AVIRE_P03360_3mut |
| GGSGGG | 13,154 | MLVMS_P03355_3mutA_WS |
| GGSGSSGGG | 13,155 | MLVMS_P03355_PLV919 |
| GGGGSGGGGSGGGGSGGGGSGGGGSGGGGS | 13,156 | KORV_Q9TTC1_3mut |
| GGGGSEAAAKGGGGS | 13,157 | KORV_Q9TTC1_3mutA |
| PAPAPAPAPAP | 13,158 | FLV_P10273_3mutA |
| GGS | | MLVBM_Q7SVK7_3mutA_WS |
| GGGGGSEAAAK | 13,160 | MLVBM_Q7SVK7_3mutA_WS |
| GSSGSSGSSGSSGSS | 13,161 | MLVMS_P03355_3mutA_WS |
| EAAAKEAAAKEAAAKEAAAKEAAAK | 13,162 | MLVMS_P03355_3mut |
| GGSGSSGGG | 13,163 | PERV_Q4VFZ2_3mut |
| PAP | | MLVFF_P26809_3mut |
| GSSPAPEAAAK | 13,165 | MLVAV_P03356_3mutA |
| EAAAKGGGGSS | 13,166 | MLVMS_P03355_3mut |
| GGGEAAAKGGS | 13,167 | XMRV6_A1Z651_3mut |
| GGSGGGPAP | 13,168 | MLVBM_Q7SVK7_3mutA_WS |
| GSAGSAAGSGEF | 13,169 | BAEVM_P10272_3mutA |
| GSSEAAAK | 13,170 | MLVCB_P08361_3mut |
| PAPGSS | 13,171 | MLVMS_P03355_3mut |
| EAAAKEAAAKEAAAK | 13,172 | MLVAV_P03356_3mutA |
| GSAGSAAGSGEF | 13,173 | XMRV6_A1Z651_3mutA |
| GSSGSSGSSGSS | 13,174 | BAEVM_P10272_3mutA |
| AEAAAKEAAAKEAAAKEAAAKALEAEAAAKEAAAKEAAAKEAAAKA | 13,175 | KORV_Q9TTC1-Pro_3mut |
| GGGGSSEAAAK | 13,176 | WMSV_P03359_3mut |
| GSSGGGEAAAK | 13,177 | MLVBM_Q7SVK7_3mutA_WS |
| EAAAKPAP | 13,178 | MLVFF_P26809_3mutA |
| GGSPAPGGG | 13,179 | KORV_Q9TTC1_3mutA |
| PAPEAAAK | 13,180 | FLV_P10273_3mutA |
| GSSGSSGSS | 13,181 | MLVBM_Q7SVK7_3mutA_WS |
| GSSGGGEAAAK | 13,182 | FLV_P10273_3mutA |
| GGSPAP | 13,183 | MLVBM_Q7SVK7_3mutA_WS |
| GSAGSAAGSGEF | 13,184 | KORV_Q9TTC1-Pro_3mutA |
| PAPGGSEAAAK | 13,185 | MLVMS_P03355_PLV919 |
| GGSPAPEAAAK | 13,186 | MLVBM_Q7SVK7_3mutA_WS |
| GGGGGSPAP | 13,187 | MLVBM_Q7SVK7_3mutA_WS |
| EAAAKGSSPAP | 13,188 | WMSV_P03359_3mut |
| EAAAKGGGPAP | 13,189 | MLVBM_Q7SVK7_3mutA_WS |
| PAPGSS | 13,190 | KORV_Q9TTC1-Pro_3mutA |
| GGSGSSGGG | 13,191 | BAEVM_P10272_3mut |
| SGGSSGGSSGSETPGTSESATPESSGGSSGGSS | 13,192 | FFV_O93209-Pro_2mut |
| GGSGGSGGSGGSGGSGGS | 13,193 | WMSV_P03359_3mutA |
| GGSGGSGGS | 13,194 | PERV_Q4VFZ2_3mutA_WS |
| GGGGG | 13,195 | PERV_Q4VFZ2_3mutA_WS |
| GGGPAP | 13,196 | FLV_P10273_3mutA |
| PAPGGSGGG | 13,197 | XMRV6_A1Z651_3mutA |
| GGGGSEAAAKGGGGS | 13,198 | XMRV6_A1Z651_3mut |
| EAAAKGSSGGG | 13,199 | KORV_Q9TTC1-Pro_3mutA |
| GSSGGSEAAAK | 13,200 | WMSV_P03359_3mut |
| EAAAKGGSGSS | 13,201 | PERV_Q4VFZ2_3mut |
| PAPAPAPAPAP | 13,202 | PERV_Q4VFZ2_3mut |
| GGGGSGGGGSGGGGSGGGGSGGGGSGGGGS | 13,203 | MLVMS_P03355_3mutA_WS |
| GGGGGGG | 13,204 | KORV_Q9TTC1_3mutA |
| EAAAK | 13,205 | KORV_Q9TTC1-Pro_3mutA |
| GGGEAAAKGGS | 13,206 | KORV_Q9TTC1-Pro_3mutA |
| GGGEAAAKGGS | 13,207 | PERV_Q4VFZ2_3mutA_WS |
| GGGGGSPAP | 13,208 | XMRV6_A1Z651_3mut |
| GGGGSGGGGSGGGGSGGGGS | 13,209 | MLVFF_P26809_3mut |
| GGGGGGG | 13,210 | MLVFF_P26809_3mut |
| PAPAPAPAPAPAP | 13,211 | AVIRE_P03360_3mutA |
| GSSPAPGGG | 13,212 | FLV_P10273_3mutA |
| GGGGGSPAP | 13,213 | MLVMS_P03355_3mutA_WS |
| GGGGSGGGGSGGGGS | 13,214 | MLVMS_P03355_3mut |
| GGGGSGGGGSGGGGS | 13,215 | KORV_Q9TTC1_3mut |
| GSSEAAAKGGS | 13,216 | MLVAV_P03356_3mutA |
| GSSGSSGSSGSSGSS | 13,217 | MLVMS_P03355_3mut |
| EAAAKGGGGGS | 13,218 | PERV_Q4VFZ2_3mutA_WS |
| GSSGGGGGS | 13,219 | PERV_Q4VFZ2_3mut |
| GGGEAAAKPAP | 13,220 | MLVMS_P03355_3mut |
| GSSGGSPAP | 13,221 | PERV_Q4VFZ2_3mutA_WS |
| GSSGGGPAP | 13,222 | BAEVM_P10272_3mutA |
| GGGGGSGSS | 13,223 | MLVMS_P03355_PLV919 |
| AEAAAKEAAAKEAAAKEAAAKALEAEAAAKEAAAKEAAAKEAAAKA | 13,224 | BAEVM_P10272_3mut |
| PAPEAAAK | 13,225 | MLVMS_P03355_3mut |
| GGGGSGGGGSGGGGS | 13,226 | FLV_P10273_3mutA |
| GGSGSSGGG | 13,227 | WMSV_P03359_3mutA |
| EAAAKGGS | 13,228 | PERV_Q4VFZ2_3mut |
| EAAAKGSSPAP | 13,229 | MLVCB_P08361_3mut |
| EAAAKGGSGSS | 13,230 | WMSV_P03359_3mutA |
| GSSGSS | 13,231 | PERV_Q4VFZ2_3mutA_WS |
| PAPAPAPAP | 13,232 | MLVMS_P03355_PLV919 |
| GGSGGG | 13,233 | PERV_Q4VFZ2_3mutA_WS |
| GSS | | MLVBM_Q7SVK7_3mutA_WS |
| PAP | | KORV_Q9TTC1-Pro_3mutA |
| GGSGSSEAAAK | 13,236 | MLVFF_P26809_3mut |
| PAPEAAAKGSS | 13,237 | KORV_Q9TTC1-Pro_3mutA |
| GGSGGS | 13,238 | MLVCB_P08361_3mutA |
| GGGGGGG | 13,239 | PERV_Q4VFZ2_3mutA_WS |
| GGSPAPEAAAK | 13,240 | MLVBM_Q7SVK7_3mut |
| EAAAKEAAAKEAAAKEAAAKEAAAKEAAAK | 13,241 | KORV_Q9TTC1_3mutA |
| GGSPAP | 13,242 | MLVMS_P03355_3mut |
| GGSEAAAKGGG | 13,243 | PERV_Q4VFZ2_3mut |
| GGGGSGGGGS | 13,244 | FLV_P10273_3mutA |
| GGGEAAAK | 13,245 | BAEVM_P10272_3mutA |
| GGGGSGGGGSGGGGSGGGGSGGGGSGGGGS | 13,246 | SFV3L_P27401_2mut |
| GGSEAAAKPAP | 13,247 | KORV_Q9TTC1-Pro_3mutA |
| GSSGGGEAAAK | 13,248 | MLVMS_P03355_PLV919 |
| GGGGGSEAAAK | 13,249 | MLVMS_P03355_PLV919 |
| EAAAKGGSGGG | 13,250 | MLVMS_P03355_3mutA_WS |
| GGGGSSPAP | 13,251 | MLVAV_P03356_3mutA |
| EAAAKEAAAK | 13,252 | MLVMS_P03355_3mutA_WS |
| AEAAAKEAAAKEAAAKEAAAKALEAEAAAKEAAAKEAAAKEAAAKA | 13,253 | SFV3L_P27401_2mut |
| GSSGSSGSSGSSGSS | 13,254 | MLVMS_P03355_PLV919 |
| GSSGGG | 13,255 | KORV_Q9TTC1-Pro_3mutA |
| GSSGGS | 13,256 | MLVFF_P26809_3mutA |
| GGGGSGGGGS | 13,257 | XMRV6_A1Z651_3mutA |
| PAPGSS | 13,258 | MLVBM_Q7SVK7_3mutA_WS |
| GGGPAPEAAAK | 13,259 | XMRV6_A1Z651_3mutA |
| EAAAKGGS | 13,260 | MLVFF_P26809_3mut |
| GSS | | KORV_Q9TTC1_3mutA |
| GGGG | 13,262 | PERV_Q4VFZ2_3mut |
| GGGGGSEAAAK | 13,263 | AVIRE_P03360_3mutA |
| GSSGSSGSSGSSGSS | 13,264 | MLVMS_P03355_PLV919 |
| PAPGGSGGG | 13,265 | PERV_Q4VFZ2_3mut |
| GGGPAP | 13,266 | PERV_Q4VFZ2_3mut |
| GGGPAPEAAAK | 13,267 | AVIRE_P03360_3mutA |
| GGGEAAAK | 13,268 | MLVCB_P08361_3mut |
| GGG | | MLVFF_P26809_3mutA |
| EAAAKPAPGSS | 13,270 | XMRV6_A1Z651_3mutA |
| GGSGSSEAAAK | 13,271 | PERV_Q4VFZ2_3mutA_WS |
| EAAAKGSS | 13,272 | MLVMS_P03355_3mut |
| GGSGSSEAAAK | 13,273 | BAEVM_P10272_3mut |
| GGSGGG | 13,274 | MLVBM_Q7SVK7_3mutA_WS |
| GGGPAP | 13,275 | MLVMS_P03355_PLV919 |
| GGSPAPGGG | 13,276 | PERV_Q4VFZ2_3mutA_WS |
| GGGGGSEAAAK | 13,277 | MLVFF_P26809_3mutA |
| EAAAKGSSGGS | 13,278 | MLVBM_Q7SVK7_3mut |
| PAPAP | 13,279 | XMRV6_A1Z651_3mut |
| GSSPAPGGS | 13,280 | MLVBM_Q7SVK7_3mutA_WS |
| GSSEAAAKGGG | 13,281 | WMSV_P03359_3mutA |
| EAAAKGGGGGS | 13,282 | PERV_Q4VFZ2_3mut |
| GSSGSSGSSGSSGSS | 13,283 | MLVCB_P08361_3mutA |
| EAAAKGGGGSS | 13,284 | PERV_Q4VFZ2_3mut |
| EAAAKGSS | 13,285 | PERV_Q4VFZ2_3mut |
| EAAAKEAAAKEAAAKEAAAKEAAAKEAAAK | 13,286 | AVIRE_P03360_3mutA |
| EAAAKGGS | 13,287 | MLVCB_P08361_3mut |
| GSSGGSEAAAK | 13,288 | MLVAV_P03356_3mutA |
| EAAAKPAPGGS | 13,289 | PERV_Q4VFZ2_3mut |
| GGSGGS | 13,290 | MLVAV_P03356_3mutA |
| EAAAKGSSGGG | 13,291 | AVIRE_P03360_3mutA |
| GGSGGSGGSGGS | 13,292 | PERV_Q4VFZ2_3mut |
| GGGGGGGG | 13,293 | KORV_Q9TTC1_3mutA |
| GGSGSSEAAAK | 13,294 | MLVCB_P08361_3mutA |
| EAAAKGGG | 13,295 | MLVBM_Q7SVK7_3mutA_WS |
| GGGGSGGGGSGGGGS | 13,296 | MLVCB_P08361_3mut |
| GGSGGSGGSGGS | 13,297 | PERV_Q4VFZ2_3mutA_WS |
| PAPAPAPAPAP | 13,298 | WMSV_P03359_3mut |
| EAAAKEAAAKEAAAKEAAAK | 13,299 | PERV_Q4VFZ2_3mut |
| GGSGGSGGS | 13,300 | XMRV6_A1Z651_3mutA |
| PAPGGGGSS | 13,301 | BAEVM_P10272_3mutA |
| GSSEAAAKGGS | 13,302 | MLVCB_P08361_3mut |
| GSSGGGPAP | 13,303 | MLVCB_P08361_3mutA |
| GGSGSS | 13,304 | MLVBM_Q7SVK7_3mutA_WS |
| GGGGGSEAAAK | 13,305 | MLVAV_P03356_3mutA |
| GSSEAAAK | 13,306 | PERV_Q4VFZ2_3mutA_WS |
| GGGGGSGSS | 13,307 | MLVBM_Q7SVK7_3mutA_WS |
| EAAAKGGSGSS | 13,308 | MLVFF_P26809_3mut |
| PAP | | FLV_P10273_3mutA |
| GGGGG | 13,310 | MLVMS_P03355_3mutA_WS |
| EAAAK | 13,311 | PERV_Q4VFZ2_3mut |
| GSS | | FLV_P10273_3mutA |
| PAPAPAPAPAPAP | 13,313 | KORV_Q9TTC1-Pro_3mutA |
| EAAAKEAAAKEAAAKEAAAK | 13,314 | MLVCB_P08361_3mut |
| EAAAKGGGGSEAAAK | 13,315 | XMRV6_A1Z651_3mut |
| PAPGGSGGG | 13,316 | MLVBM_Q7SVK7_3mutA_WS |
| GGSGGGPAP | 13,317 | WMSV_P03359_3mutA |
| GGGGSSEAAAK | 13,318 | MLVBM_Q7SVK7_3mutA_WS |
| PAPGGGGSS | 13,319 | MLVCB_P08361_3mut |
| GGSGGSGGSGGS | 13,320 | PERV_Q4VFZ2_3mutA_WS |
| PAPGGSGGG | 13,321 | MLVMS_P03355_3mutA_WS |
| GSSPAPGGS | 13,322 | MLVCB_P08361_3mutA |
| GSSGSSGSS | 13,323 | MLVFF_P26809_3mut |
| PAPGGGGGS | 13,324 | MLVBM_Q7SVK7_3mutA_WS |
| GSSPAP | 13,325 | PERV_Q4VFZ2_3mut |
| GGSGGG | 13,326 | KORV_Q9TTC1-Pro_3mut |
| EAAAKGGGGSEAAAK | 13,327 | PERV_Q4VFZ2_3mutA_WS |
| GGSPAPEAAAK | 13,328 | PERV_Q4VFZ2_3mutA_WS |
| EAAAKPAP | 13,329 | BAEVM_P10272_3mut |
| GGGGSGGGGSGGGGSGGGGSGGGGSGGGGS | 13,330 | MLVMS_P03355_3mut |
| EAAAKGGGGSS | 13,331 | MLVFF_P26809_3mut |
| EAAAKEAAAK | 13,332 | MLVCB_P08361_3mut |
| GSSEAAAKGGS | 13,333 | PERV_Q4VFZ2_3mut |
| GGSPAP | 13,334 | KORV_Q9TTC1-Pro_3mutA |
| EAAAKEAAAKEAAAKEAAAK | 13,335 | MLVMS_P03355_3mutA_WS |
| GSSGSSGSSGSSGSS | 13,336 | BAEVM_P10272_3mut |
| PAPEAAAK | 13,337 | MLVMS_P03355_3mut |
| GSSGGSPAP | 13,338 | PERV_Q4VFZ2 |
| GGGPAPGGS | 13,339 | BAEVM_P10272_3mutA |
| EAAAKPAPGGS | 13,340 | MLVMS_P03355_PLV919 |
| GGGGSGGGGS | 13,341 | PERV_Q4VFZ2 |
| GGGEAAAK | 13,342 | KORV_Q9TTC1-Pro_3mut |
| EAAAKGGGGGS | 13,343 | FLV_P10273_3mutA |
| GGSPAPGSS | 13,344 | MLVMS_P03355_3mut |
| GSSPAPEAAAK | 13,345 | MLVMS_P03355_3mutA_WS |
| GSAGSAAGSGEF | 13,346 | MLVBM_Q7SVK7_3mutA_WS |
| EAAAK | 13,347 | BAEVM_P10272_3mutA |
| EAAAKGGGGSS | 13,348 | BAEVM_P10272_3mutA |
| GGG | | WMSV_P03359_3mut |
| GGSGSSPAP | 13,350 | BAEVM_P10272_3mut |
| GGSEAAAKPAP | 13,351 | MLVBM_Q7SVK7_3mutA_WS |
| EAAAKGGSGSS | 13,352 | MLVCB_P08361_3mut |
| PAPGSS | 13,353 | MLVAV_P03356_3mutA |
| PAPEAAAKGGG | 13,354 | MLVCB_P08361_3mutA |
| AEAAAKEAAAKEAAAKEAAAKALEAEAAAKEAAAKEAAAKEAAAKA | 13,355 | FOAMV_P14350-Pro_2mut |
| GSSGSSGSS | 13,356 | PERV_Q4VFZ2_3mut |
| PAPGGG | 13,357 | MLVMS_P03355_3mut |
| PAPGGS | 13,358 | PERV_Q4VFZ2_3mut |
| GSSGGG | 13,359 | MLVMS_P03355_PLV919 |
| GSSGSSGSSGSSGSSGSS | 13,360 | WMSV_P03359_3mut |
| PAP | | AVIRE_P03360_3mutA |
| EAAAKGSSPAP | 13,362 | MLVBM_Q7SVK7_3mutA_WS |
| GSSGSSGSSGSS | 13,363 | MLVMS_P03355_PLV919 |
| GGGGSGGGGSGGGGSGGGGSGGGGS | 13,364 | AVIRE_P03360 |
| GGGGS | 13,365 | PERV_Q4VFZ2_3mut |
| EAAAKGSSGGG | 13,366 | MLVBM_Q7SVK7_3mutA_WS |
| GGGGGG | 13,367 | KORV_Q9TTC1-Pro_3mut |
| GGSGSSEAAAK | 13,368 | PERV_Q4VFZ2_3mut |
| GSSPAPEAAAK | 13,369 | MLVBM_Q7SVK7_3mutA_WS |
| GGGGSGGGGS | 13,370 | MLVBM_Q7SVK7_3mutA_WS |
| GSSGGGGGS | 13,371 | MLVAV_P03356_3mutA |
| GSAGSAAGSGEF | 13,372 | WMSV_P03359_3mutA |
| GGGEAAAKGSS | 13,373 | BAEVM_P10272_3mutA |
| AEAAAKEAAAKEAAAKEAAAKALEAEAAAKEAAAKEAAAKEAAAKA | 13,374 | FFV_O93209-Pro_2mut |
| PAPGGSGGG | 13,375 | MLVCB_P08361_3mutA |
| EAAAKEAAAKEAAAKEAAAKEAAAK | 13,376 | SFV3L_P27401_2mut |
| GGSGSSPAP | 13,377 | MLVMS_P03355_PLV919 |
| GGGGGG | 13,378 | PERV_Q4VFZ2_3mut |
| EAAAKEAAAKEAAAKEAAAKEAAAK | 13,379 | PERV_Q4VFZ2_3mut |
| EAAAKGSSPAP | 13,380 | MLVFF_P26809_3mut |
| GGGPAPGGS | 13,381 | MLVBM_Q7SVK7_3mutA_WS |
| AEAAAKEAAAKEAAAKEAAAKALEAEAAAKEAAAKEAAAKEAAAKA | 13,382 | SFV3L_P27401 |
| PAP | | PERV_Q4VFZ2_3mut |
| EAAAKGGS | 13,384 | MLVMS_P03355_PLV919 |
| GSSGGSEAAAK | 13,385 | WMSV_P03359_3mutA |
| GGSGSSEAAAK | 13,386 | KORV_Q9TTC1-Pro_3mutA |
| EAAAKEAAAKEAAAK | 13,387 | PERV_Q4VFZ2 |
| GGSGGGEAAAK | 13,388 | MLVMS_P03355_3mutA_WS |
| GGGGSGGGGSGGGGSGGGGS | 13,389 | BAEVM_P10272_3mut |
| EAAAKGSS | 13,390 | XMRV6_A1Z651_3mutA |
| GSSGGGGGS | 13,391 | WMSV_P03359_3mutA |
| GSSGSSGSSGSSGSSGSS | 13,392 | MLVFF_P26809_3mutA |
| GGSGSS | 13,393 | MLVAV_P03356_3mutA |
| EAAAKGGGGSEAAAK | 13,394 | MLVMS_P03355_PLV919 |
| EAAAKGGGPAP | 13,395 | PERV_Q4VFZ2 |
| GGSEAAAKGGG | 13,396 | MLVAV_P03356_3mutA |
| EAAAKEAAAKEAAAKEAAAKEAAAKEAAAK | 13,397 | MLVBM_Q7SVK7_3mut |
| EAAAKEAAAKEAAAKEAAAKEAAAKEAAAK | 13,398 | KORV_Q9TTC1-Pro_3mutA |
| GSSPAPEAAAK | 13,399 | MLVFF_P26809_3mutA |
| GGGGSEAAAKGGGGS | 13,400 | PERV_Q4VFZ2_3mut |
| GSSGSSGSSGSS | 13,401 | PERV_Q4VFZ2_3mut |
| GGSEAAAK | 13,402 | MLVFF_P26809_3mutA |
| GGGGGGGG | 13,403 | MLVMS_P03355_3mut |
| GSSGGG | 13,404 | XMRV6_A1Z651_3mutA |
| EAAAKGGS | 13,405 | BAEVM_P10272_3mutA |
| GGGGS | 13,406 | BAEVM_P10272_3mutA |
| GGSEAAAKGGG | 13,407 | KORV_Q9TTC1-Pro_3mutA |
| GGSGSSGGG | 13,408 | KORV_Q9TTC1_3mutA |
| GGSGSSEAAAK | 13,409 | WMSV_P03359_3mut |
| EAAAKGGSGSS | 13,410 | MLVBM_Q7SVK7_3mutA_WS |
| GGS | | BAEVM_P10272_3mutA |
| GGGPAPGSS | 13,412 | WMSV_P03359_3mutA |
| GSSGSSGSSGSSGSS | 13,413 | AVIRE_P03360_3mut |
| GGGEAAAKPAP | 13,414 | XMRV6_A1Z651_3mut |
| GSSGGG | 13,415 | MLVFF_P26809_3mutA |
| GGSPAPGSS | 13,416 | PERV_Q4VFZ2_3mut |
| PAPGGS | 13,417 | MLVCB_P08361_3mut |
| PAPAPAPAPAP | 13,418 | KORV_Q9TTC1_3mutA |
| GSSGGS | 13,419 | MLVCB_P08361_3mutA |
| GSSGGSEAAAK | 13,420 | PERV_Q4VFZ2_3mut |
| EAAAKGSSGGS | 13,421 | MLVMS_P03355_PLV919 |
| EAAAKGGG | 13,422 | WMSV_P03359_3mut |
| PAPGGGGGS | 13,423 | BAEVM_P10272_3mutA |
| GGGGSEAAAKGGGGS | 13,424 | WMSV_P03359_3mutA |
| EAAAKEAAAKEAAAKEAAAKEAAAKEAAAK | 13,425 | MLVMS_P03355_3mutA_WS |
| GGS | | KORV_Q9TTC1-Pro_3mutA |
| GSSGGSPAP | 13,427 | BAEVM_P10272_3mutA |
| GGG | | MLVMS_P03355_PLV919 |
| PAPGSS | 13,429 | KORV_Q9TTC1-Pro_3mut |
| GGSEAAAKGGG | 13,430 | FLV_P10273_3mutA |
| GGSEAAAKPAP | 13,431 | PERV_Q4VFZ2_3mutA_WS |
| GGGGSSPAP | 13,432 | XMRV6_A1Z651_3mutA |
| EAAAKEAAAKEAAAKEAAAKEAAAK | 13,433 | PERV_Q4VFZ2_3mutA_WS |
| GGGG | 13,434 | PERV_Q4VFZ2_3mutA_WS |
| GGSEAAAKPAP | 13,435 | MLVMS_P03355_3mut |
| PAPGSSGGG | 13,436 | MLVMS_P03355_3mutA_WS |
| PAPEAAAKGGS | 13,437 | AVIRE_P03360_3mut |
| GGGGSSPAP | 13,438 | MLVMS_P03355_3mutA_WS |
| GGGGSGGGGSGGGGSGGGGS | 13,439 | PERV_Q4VFZ2_3mut |
| GGGEAAAK | 13,440 | MLVMS_P03355_3mut |
| GGGGSS | 13,441 | MLVFF_P26809_3mut |
| GGSPAPGSS | 13,442 | XMRV6_A1Z651_3mut |
| GGGGS | 13,443 | KORV_Q9TTC1-Pro_3mutA |
| EAAAKGSSGGS | 13,444 | FLV_P10273_3mutA |
| GSS | | MLVMS_P03355_PLV919 |
| GGGG | 13,446 | MLVMS_P03355_PLV919 |
| GSSGGS | 13,447 | MLVMS_P03355_PLV919 |
| GGSGGSGGSGGS | 13,448 | MLVMS_P03355_3mut |
| PAPEAAAKGGS | 13,449 | MLVMS_P03355_3mut |
| EAAAKGSSGGG | 13,450 | BAEVM_P10272_3mutA |
| GSSEAAAK | 13,451 | KORV_Q9TTC1-Pro_3mutA |
| GSAGSAAGSGEF | 13,452 | KORV_Q9TTC1_3mutA |
| GGGGGSEAAAK | 13,453 | MLVCB_P08361_3mut |
| GGGG | 13,454 | WMSV_P03359_3mut |
| GGGGSSEAAAK | 13,455 | MLVMS_P03355_PLV919 |
| PAPGGG | 13,456 | WMSV_P03359_3mutA |
| EAAAKGGSGGG | 13,457 | MLVAV_P03356_3mutA |
| GGGPAPGGS | 13,458 | MLVMS_P03355_3mut |
| EAAAKPAP | 13,459 | PERV_Q4VFZ2_3mutA_WS |
| GSSGSSGSS | 13,460 | KORV_Q9TTC1-Pro_3mutA |
| GSSPAPGGS | 13,461 | XMRV6_A1Z651_3mut |
| GGGGGSPAP | 13,462 | BAEVM_P10272_3mutA |
| GGSGSSGGG | 13,463 | PERV_Q4VFZ2_3mutA_WS |
| GGGEAAAKGSS | 13,464 | AVIRE_P03360_3mut |
| GSSEAAAK | 13,465 | FLV_P10273_3mutA |
| EAAAK | 13,466 | MLVMS_P03355_3mut |
| EAAAKGGSGSS | 13,467 | WMSV_P03359_3mut |
| GSSEAAAKGGG | 13,468 | PERV_Q4VFZ2_3mut |
| PAPGSSGGG | 13,469 | BAEVM_P10272_3mutA |
| EAAAKGGGGGS | 13,470 | MLVMS_P03355_3mut |
| GGSEAAAKPAP | 13,471 | AVIRE_P03360_3mut |
| GGGPAPGGS | 13,472 | XMRV6_A1Z651_3mut |
| GGGGS | 13,473 | KORV_Q9TTC1_3mutA |
| GGSGGSGGSGGSGGS | 13,474 | XMRV6_A1Z651_3mut |
| GGGPAP | 13,475 | KORV_Q9TTC1-Pro_3mut |
| EAAAKPAP | 13,476 | MLVBM_Q7SVK7_3mutA_WS |
| GGSEAAAK | 13,477 | MLVMS_P03355_PLV919 |
| GSSEAAAKPAP | 13,478 | KORV_Q9TTC1-Pro_3mutA |
| GGSGSS | 13,479 | MLVMS_P03355_3mut |
| EAAAKPAPGGG | 13,480 | PERV_Q4VFZ2_3mut |
| GGSPAPEAAAK | 13,481 | KORV_Q9TTC1_3mutA |
| GGSEAAAKGGG | 13,482 | AVIRE_P03360_3mutA |
| GGGGSEAAAKGGGGS | 13,483 | MLVMS_P03355_PLV919 |
| GSSGGGEAAAK | 13,484 | KORV_Q9TTC1-Pro_3mutA |
| EAAAKGGGPAP | 13,485 | WMSV_P03359_3mut |
| GSSPAP | 13,486 | XMRV6_A1Z651_3mutA |
| AEAAAKEAAAKEAAAKEAAAKALEAEAAAKEAAAKEAAAKEAAAKA | 13,487 | SFV3L_P27401-Pro |
| GGSEAAAKGSS | 13,488 | MLVMS_P03355_PLV919 |
| GSSGGSEAAAK | 13,489 | KORV_Q9TTC1-Pro_3mutA |
| GGSEAAAKGSS | 13,490 | KORV_Q9TTC1-Pro_3mutA |
| EAAAKGGG | 13,491 | AVIRE_P03360_3mutA |
| GSSGGSEAAAK | 13,492 | BAEVM_P10272_3mutA |
| GGGGSEAAAKGGGGS | 13,493 | KORV_Q9TTC1-Pro_3mut |
| PAPGSSEAAAK | 13,494 | MLVMS_P03355_3mut |
| PAPEAAAK | 13,495 | WMSV_P03359_3mut |
| PAPGGSGSS | 13,496 | PERV_Q4VFZ2_3mutA_WS |
| PAPGSS | 13,497 | BAEVM_P10272_3mut |
| PAPGGGGGS | 13,498 | MLVMS_P03355_3mut |
| EAAAKPAPGSS | 13,499 | MLVBM_Q7SVK7_3mutA_WS |
| GSSPAPGGS | 13,500 | MLVMS_P03355_PLV919 |
| GGSGSSEAAAK | 13,501 | MLVMS_P03355_3mut |
| GGGGGG | 13,502 | KORV_Q9TTC1-Pro_3mutA |
| EAAAKEAAAKEAAAKEAAAK | 13,503 | MLVBM_Q7SVK7_3mut |
| GGSPAPGSS | 13,504 | MLVMS_P03355_PLV919 |
| PAPAPAPAPAP | 13,505 | MLVCB_P08361_3mut |
| GGSGSSPAP | 13,506 | WMSV_P03359_3mutA |
| EAAAKGGSGGG | 13,507 | PERV_Q4VFZ2_3mutA_WS |
| GSSGSSGSSGSSGSS | 13,508 | PERV_Q4VFZ2_3mut |
| AEAAAKEAAAKEAAAKEAAAKALEAEAAAKEAAAKEAAAKEAAAKA | 13,509 | KORV_Q9TTC1_3mutA |
| GSSGGGEAAAK | 13,510 | WMSV_P03359_3mutA |
| GSSGGSEAAAK | 13,511 | FLV_P10273_3mutA |
| GGGGGGGG | 13,512 | PERV_Q4VFZ2_3mut |
| PAPGGSEAAAK | 13,513 | FLV_P10273_3mutA |
| GGGGSSPAP | 13,514 | BAEVM_P10272_3mutA |
| PAPAPAPAP | 13,515 | WMSV_P03359_3mut |
| GGSEAAAKPAP | 13,516 | PERV_Q4VFZ2_3mut |
| PAPGGSGGG | 13,517 | BAEVM_P10272_3mutA |
| EAAAKEAAAKEAAAKEAAAKEAAAKEAAAK | 13,518 | MLVMS_P03355_3mut |
| GGGGSGGGGSGGGGS | 13,519 | PERV_Q4VFZ2_3mut |
| GGSGGGPAP | 13,520 | PERV_Q4VFZ2_3mut |
| GGGPAPEAAAK | 13,521 | MLVFF_P26809_3mut |
| GGGGGSGSS | 13,522 | MLVMS_P03355_3mutA_WS |
| GSS | | MLVCB_P08361_3mut |
| GGGGGSPAP | 13,524 | MLVMS_P03355_PLV919 |
| GGSPAP | 13,525 | MLVAV_P03356_3mutA |
| GGGPAPGGS | 13,526 | KORV_Q9TTC1-Pro_3mutA |
| PAPGSSGGG | 13,527 | FLV_P10273_3mutA |
| PAPGSSGGG | 13,528 | WMSV_P03359_3mutA |
| PAPGGS | 13,529 | MLVBM_Q7SVK7_3mutA_WS |
| GGGEAAAKGSS | 13,530 | PERV_Q4VFZ2_3mutA_WS |
| GGSEAAAKGSS | 13,531 | MLVBM_Q7SVK7_3mutA_WS |
| PAPGGSEAAAK | 13,532 | MLVCB_P08361_3mut |
| GGSEAAAKGGG | 13,533 | XMRV6_A1Z651_3mutA |
| GGSGGGGSS | 13,534 | WMSV_P03359_3mut |
| GGGEAAAKPAP | 13,535 | KORV_Q9TTC1_3mutA |
| EAAAKGSS | 13,536 | KORV_Q9TTC1-Pro_3mut |
| PAPEAAAKGSS | 13,537 | MLVFF_P26809_3mut |
| GSAGSAAGSGEF | 13,538 | PERV_Q4VFZ2_3mut |
| EAAAKGGGGGS | 13,539 | WMSV_P03359_3mut |
| EAAAKGSSPAP | 13,540 | WMSV_P03359_3mutA |
| GGGGSEAAAKGGGGS | 13,541 | XMRV6_A1Z651_3mutA |
| GSSEAAAKPAP | 13,542 | SFV3L_P27401-Pro_2mutA |
| GGGGGG | 13,543 | PERV_Q4VFZ2_3mutA_WS |
| PAPGGS | 13,544 | BAEVM_P10272_3mut |
| PAP | | AVIRE_P03360_3mut |
| PAPAPAP | 13,546 | MLVBM_Q7SVK7_3mutA_WS |
| GGGG | 13,547 | PERV_Q4VFZ2_3mutA_WS |
| GSSGGSEAAAK | 13,548 | MLVBM_Q7SVK7_3mut |
| GGSGGGGSS | 13,549 | MLVFF_P26809_3mut |
| GGGGSSGGS | 13,550 | AVIRE_P03360_3mutA |
| GSSPAPGGG | 13,551 | PERV_Q4VFZ2_3mutA_WS |
| GGSEAAAKPAP | 13,552 | MLVMS_P03355_PLV919 |
| PAP | | KORV_Q9TTC1-Pro_3mut |
| GSSGGS | 13,554 | PERV_Q4VFZ2_3mut |
| GGGGG | 13,555 | PERV_Q4VFZ2_3mut |
| GSSGGGPAP | 13,556 | FLV_P10273_3mutA |
| GSSEAAAKGGG | 13,557 | KORV_Q9TTC1-Pro_3mut |
| EAAAKEAAAKEAAAKEAAAKEAAAKEAAAK | 13,558 | MLVCB_P08361_3mut |
| GGSEAAAKPAP | 13,559 | MLVCB_P08361_3mut |
| PAPAPAPAPAPAP | 13,560 | BAEVM_P10272_3mutA |
| GGGGSEAAAKGGGGS | 13,561 | MLVMS_P03355_3mut |
| EAAAKPAPGSS | 13,562 | MLVMS_P03355_3mut |
| GSSGSSGSSGSSGSS | 13,563 | MLVBM_Q7SVK7_3mutA_WS |
| PAPEAAAKGSS | 13,564 | MLVAV_P03356_3mut |
| AEAAAKEAAAKEAAAKEAAAKALEAEAAAKEAAAKEAAAKEAAAKA | 13,565 | AVIRE_P03360_3mut |
| AEAAAKEAAAKEAAAKEAAAKALEAEAAAKEAAAKEAAAKEAAAKA | 13,566 | PERV_Q4VFZ2_3mut |
| GGSEAAAKGGG | 13,567 | PERV_Q4VFZ2_3mutA_WS |
| GGSGGGGSS | 13,568 | MLVFF_P26809_3mutA |
| PAPEAAAKGSS | 13,569 | MLVCB_P08361_3mut |
| GGG | | PERV_Q4VFZ2_3mutA_WS |
| GGSGGGEAAAK | 13,571 | MLVMS_P03355_3mut |
| EAAAKGGGGSS | 13,572 | WMSV_P03359_3mut |
| GSSPAPGGG | 13,573 | WMSV_P03359_3mutA |
| EAAAKGSSGGG | 13,574 | PERV_Q4VFZ2_3mut |
| GGSGGGEAAAK | 13,575 | PERV_Q4VFZ2_3mutA_WS |
| GGSGGSGGSGGSGGS | 13,576 | PERV_Q4VFZ2_3mutA_WS |
| EAAAKPAPGGS | 13,577 | PERV_Q4VFZ2_3mutA_WS |
| GGGGGSEAAAK | 13,578 | PERV_Q4VFZ2_3mutA_WS |
| GSSPAP | 13,579 | MLVFF_P26809_3mut |
| GGGEAAAKPAP | 13,580 | AVIRE_P03360_3mut |
| GSSGGSEAAAK | 13,581 | MLVMS_P03355_PLV919 |
| EAAAKPAPGGS | 13,582 | WMSV_P03359_3mutA |
| PAPGGG | 13,583 | KORV_Q9TTC1_3mutA |
| EAAAKGSSPAP | 13,584 | KORV_Q9TTC1-Pro_3mut |
| GSSPAPEAAAK | 13,585 | MLVFF_P26809_3mut |
| GGSGGGEAAAK | 13,586 | MLVFF_P26809_3mutA |
| GSSGSSGSS | 13,587 | WMSV_P03359_3mutA |
| EAAAKGGS | 13,588 | BAEVM_P10272_3mut |
| EAAAKPAPGGS | 13,589 | KORV_Q9TTC1_3mutA |
| EAAAKPAPGGS | 13,590 | BAEVM_P10272_3mutA |
| GSSGGGGGS | 13,591 | PERV_Q4VFZ2_3mut |
| PAPGGGGSS | 13,592 | PERV_Q4VFZ2_3mut |
| GSSGSSGSS | 13,593 | WMSV_P03359_3mut |
| EAAAKEAAAKEAAAKEAAAK | 13,594 | WMSV_P03359_3mut |
| GGS | | AVIRE_P03360_3mut |
| EAAAKPAPGSS | 13,596 | MLVFF_P26809_3mut |
| EAAAKGGG | 13,597 | KORV_Q9TTC1_3mut |
| PAPGSSEAAAK | 13,598 | MLVMS_P03355_3mut |
| PAPGSSGGS | 13,599 | MLVMS_P03355_PLV919 |
| GSSPAPEAAAK | 13,600 | MLVMS_P03355_3mut |
| GSSGSSGSSGSSGSSGSS | 13,601 | WMSV_P03359_3mutA |
| GGGGS | 13,602 | BAEVM_P10272_3mut |
| GSSPAP | 13,603 | MLVMS_P03355_3mut |
| EAAAKGGGGSEAAAK | 13,604 | KORV_Q9TTC1-Pro_3mutA |
| EAAAKEAAAK | 13,605 | WMSV_P03359_3mutA |
| GGGGSSGGS | 13,606 | MLVCB_P08361_3mutA |
| PAPGGSEAAAK | 13,607 | BAEVM_P10272_3mut |
| EAAAKGGSPAP | 13,608 | MLVFF_P26809_3mut |
| GSSGGSGGG | 13,609 | MLVBM_Q7SVK7_3mutA_WS |
| GSSGGS | 13,610 | PERV_Q4VFZ2_3mut |
| PAPGGSGSS | 13,611 | PERV_Q4VFZ2_3mutA_WS |
| EAAAKGGSGSS | 13,612 | KORV_Q9TTC1-Pro_3mutA |
| PAPAP | 13,613 | MLVCB_P08361_3mut |
| EAAAKGSSPAP | 13,614 | PERV_Q4VFZ2_3mutA_WS |
| EAAAKPAPGGG | 13,615 | MLVMS_P03355_PLV919 |
| GGGGSGGGGSGGGGSGGGGSGGGGSGGGGS | 13,616 | MLVBM_Q7SVK7_3mut |
| EAAAKGGGGSS | 13,617 | MLVMS_P03355_PLV919 |
| PAPEAAAK | 13,618 | PERV_Q4VFZ2_3mut |
| EAAAKPAPGSS | 13,619 | BAEVM_P10272_3mutA |
| GGSPAP | 13,620 | PERV_Q4VFZ2_3mutA_WS |
| GGSGGS | 13,621 | BAEVM_P10272_3mutA |
| PAPEAAAKGSS | 13,622 | KORV_Q9TTC1_3mut |
| PAPGSS | 13,623 | MLVMS_P03355_PLV919 |
| PAPAPAPAPAP | 13,624 | MLVAV_P03356_3mutA |
| GGG | | XMRV6_A1Z651_3mutA |
| GGGPAP | 13,626 | PERV_Q4VFZ2_3mutA_WS |
| GSSPAPEAAAK | 13,627 | KORV_Q9TTC1_3mutA |
| PAP | | BAEVM_P10272_3mutA |
| GGSPAP | 13,629 | BAEVM_P10272_3mutA |
| PAPEAAAKGGS | 13,630 | MLVMS_P03355_PLV919 |
| PAPGSSGGS | 13,631 | PERV_Q4VFZ2_3mutA_WS |
| PAPAPAPAPAPAP | 13,632 | PERV_Q4VFZ2_3mut |
| EAAAKEAAAKEAAAK | 13,633 | MLVCB_P08361_3mut |
| GGSGGSGGSGGSGGS | 13,634 | MLVMS_P03355_PLV919 |
| EAAAKPAPGGS | 13,635 | MLVMS_P03355_3mut |
| GGSGGS | 13,636 | MLVMS_P03355_PLV919 |
| EAAAKPAP | 13,637 | MLVMS_P03355_3mutA_WS |
| GGSEAAAK | 13,638 | XMRV6_A1Z651_3mutA |
| GGSGGG | 13,639 | KORV_Q9TTC1_3mut |
| GGSGGGEAAAK | 13,640 | PERV_Q4VFZ2_3mut |
| PAPEAAAKGGG | 13,641 | AVIRE_P03360 |
| PAPAP | 13,642 | PERV_Q4VFZ2_3mut |
| GSS | | KORV_Q9TTC1-Pro_3mutA |
| EAAAKGSSGGG | 13,644 | MLVAV_P03356_3mutA |
| GGSPAPGSS | 13,645 | MLVBM_Q7SVK7_3mutA_WS |
| PAPEAAAK | 13,646 | MLVAV_P03356_3mut |
| EAAAKGGSPAP | 13,647 | BAEVM_P10272_3mutA |
| PAPAPAPAP | 13,648 | WMSV_P03359_3mutA |
| PAPGGSEAAAK | 13,649 | MLVMS_P03355_3mut |
| GGSGGSGGSGGS | 13,650 | WMSV_P03359_3mut |
| GGGGGSGSS | 13,651 | XMRV6_A1Z651_3mut |
| PAPGGSGGG | 13,652 | KORV_Q9TTC1_3mutA |
| GGS | | MLVMS_P03355_3mut |
| EAAAK | 13,654 | WMSV_P03359_3mut |
| GGGEAAAKGSS | 13,655 | MLVBM_Q7SVK7_3mutA_WS |
| GGSPAPGSS | 13,656 | MLVCB_P08361_3mut |
| GGSEAAAKPAP | 13,657 | PERV_Q4VFZ2_3mut |
| GGGGSGGGGSGGGGSGGGGSGGGGS | 13,658 | MLVCB_P08361_3mutA |
| GGSGSS | 13,659 | BAEVM_P10272_3mutA |
| GGGEAAAKGSS | 13,660 | WMSV_P03359_3mutA |
| EAAAKGGSPAP | 13,661 | WMSV_P03359_3mut |
| GSSPAPEAAAK | 13,662 | MLVMS_P03355_3mut |
| GGSGGSGGSGGS | 13,663 | MLVMS_P03355_PLV919 |
| GSSPAPEAAAK | 13,664 | WMSV_P03359_3mut |
| GSSGSSGSSGSS | 13,665 | PERV_Q4VFZ2 |
| GGSGSSEAAAK | 13,666 | WMSV_P03359_3mutA |
| GGSGGG | 13,667 | MLVFF_P26809_3mut |
| GGSPAPGGG | 13,668 | MLVFF_P26809_3mut |
| GGSGGSGGS | 13,669 | BAEVM_P10272_3mutA |
| GGGGSSEAAAK | 13,670 | MLVBM_Q7SVK7_3mut |
| GGSPAPGSS | 13,671 | MLVMS_P03355_3mut |
| EAAAKPAPGSS | 13,672 | AVIRE_P03360_3mut |
| GGGGSSGGS | 13,673 | FLV_P10273_3mutA |
| GGSPAPEAAAK | 13,674 | PERV_Q4VFZ2_3mut |
| GGSEAAAK | 13,675 | MLVMS_P03355_3mutA_WS |
| GSSGSSGSSGSS | 13,676 | MLVCB_P08361_3mutA |
| EAAAKEAAAKEAAAKEAAAKEAAAK | 13,677 | MLVMS_P03355_PLV919 |
| GGGGG | 13,678 | PERV_Q4VFZ2_3mut |
| GGSEAAAKGSS | 13,679 | MLVCB_P08361_3mutA |
| GSSGGG | 13,680 | MLVBM_Q7SVK7_3mutA_WS |
| PAPGSSGGG | 13,681 | KORV_Q9TTC1-Pro_3mutA |
| GGSGGS | 13,682 | BAEVM_P10272_3mut |
| EAAAKGGGGGS | 13,683 | MLVBM_Q7SVK7_3mutA_WS |
| GGSGSSPAP | 13,684 | MLVCB_P08361_3mut |
| PAPGSSGGG | 13,685 | KORV_Q9TTC1 |
| PAPGGSGGG | 13,686 | MLVMS_P03355_3mut |
| GGGG | 13,687 | WMSV_P03359_3mutA |
| EAAAKGGSPAP | 13,688 | MLVCB_P08361_3mut |
| GSSGSS | 13,689 | FLV_P10273_3mutA |
| GGSEAAAKPAP | 13,690 | SFV3L_P27401_2mut |
| EAAAKGSSGGS | 13,691 | MLVAV_P03356_3mutA |
| AEAAAKEAAAKEAAAKEAAAKALEAEAAAKEAAAKEAAAKEAAAKA | 13,692 | MLVAV_P03356_3mutA |
| EAAAKGGSGSS | 13,693 | PERV_Q4VFZ2_3mutA_WS |
| GGGGG | 13,694 | MLVCB_P08361_3mut |
| GGGEAAAK | 13,695 | BAEVM_P10272_3mut |
| GGSGGSGGSGGS | 13,696 | MLVCB_P08361_3mut |
| EAAAKEAAAKEAAAKEAAAKEAAAKEAAAK | 13,697 | PERV_Q4VFZ2 |
| PAPAPAPAPAP | 13,698 | MLVMS_P03355_3mutA_WS |
| EAAAKEAAAK | 13,699 | XMRV6_A1Z651_3mut |
| GSSGGSEAAAK | 13,700 | PERV_Q4VFZ2_3mutA_WS |
| PAPGGSEAAAK | 13,701 | KORV_Q9TTC1-Pro_3mutA |
| EAAAKGGGPAP | 13,702 | MLVBM_Q7SVK7_3mutA_WS |
| PAPGGSGSS | 13,703 | PERV_Q4VFZ2 |
| SGSETPGTSESATPES | 13,704 | MLVMS_P03355_3mut |
| GGSGGS | 13,705 | MLVMS_P03355_PLV919 |
| EAAAKGGS | 13,706 | FLV_P10273_3mut |
| GGSPAPGSS | 13,707 | MLVMS_P03355_3mutA_WS |
| EAAAKEAAAKEAAAKEAAAK | 13,708 | FFV_093209_2mut |
| GSSGGSGGG | 13,709 | MLVMS_P03355_3mutA_WS |
| PAPGSSEAAAK | 13,710 | WMSV_P03359_3mut |
| PAPAPAPAPAPAP | 13,711 | KORV_Q9TTC1_3mutA |
| GGGGSS | 13,712 | BAEVM_P10272_3mut |
| GGGGSEAAAKGGGGS | 13,713 | AVIRE_P03360_3mut |
| GSSPAPEAAAK | 13,714 | KORV_Q9TTC1-Pro_3mutA |
| PAPEAAAKGGG | 13,715 | MLVBM_Q7SVK7_3mut |
| EAAAKEAAAK | 13,716 | WMSV_P03359_3mut |
| EAAAK | 13,717 | SFV3L_P27401-Pro_2mutA |
| GSSGGSGGG | 13,718 | XMRV6_A1Z651_3mutA |
| GGGEAAAKPAP | 13,719 | WMSV_P03359_3mutA |
| GGSGGS | 13,720 | MLVFF_P26809_3mutA |
| EAAAKEAAAKEAAAKEAAAKEAAAKEAAAK | 13,721 | FOAMV_P14350_2mutA |
| GGGGG | 13,722 | MLVAV_P03356_3mutA |
| GSSGGSEAAAK | 13,723 | BAEVM_P10272_3mut |
| SGGSSGGSSGSETPGTSESATPESSGGSSGGSS | 13,724 | SFV1_P23074 |
| GGSGGGPAP | 13,725 | MLVCB_P08361_3mut |
| GGSGSS | 13,726 | PERV_Q4VFZ2_3mut |
| SGSETPGTSESATPES | 13,727 | MLVFF_P26809_3mut |
| EAAAKGGSPAP | 13,728 | MLVMS_P03355_3mut |
| PAPAP | 13,729 | PERV_Q4VFZ2_3mut |
| AEAAAKEAAAKEAAAKEAAAKALEAEAAAKEAAAKEAAAKEAAAKA | 13,730 | MLVBM_Q7SVK7_3mut |
| GGGGGS | 13,731 | BAEVM_P10272_3mutA |
| EAAAKEAAAK | 13,732 | AVIRE_P03360_3mut |
| GSSGGSEAAAK | 13,733 | PERV_Q4VFZ2_3mut |
| GGGEAAAK | 13,734 | WMSV_P03359_3mut |
| GSSGGGEAAAK | 13,735 | AVIRE_P03360_3mutA |
| GGG | | XMRV6_A1Z651_3mut |
| GGGGSEAAAKGGGGS | 13,737 | BAEVM_P10272_3mut |
| GGGG | 13,738 | MLVMS_P03355_3mut |
| GGSGGS | 13,739 | MLVMS_P03355_3mutA_WS |
| GGSGGGGSS | 13,740 | MLVBM_Q7SVK7_3mutA_WS |
| GSSPAPGGS | 13,741 | PERV_Q4VFZ2_3mut |
| GSSPAPEAAAK | 13,742 | PERV_Q4VFZ2_3mutA_WS |
| EAAAKGGS | 13,743 | WMSV_P03359_3mut |
| GGSGGSGGSGGS | 13,744 | PERV_Q4VFZ2_3mut |
| GGGGSSEAAAK | 13,745 | KORV_Q9TTC1-Pro_3mut |
| PAPAPAPAPAPAP | 13,746 | MLVAV_P03356_3mut |
| EAAAKGSSGGG | 13,747 | MLVMS_P03355_PLV919 |
| GGGGG | 13,748 | MLVBM_Q7SVK7_3mutA_WS |
| AEAAAKEAAAKEAAAKEAAAKALEAEAAAKEAAAKEAAAKEAAAKA | 13,749 | FFV_O93209_2mutA |
| SGGSSGGSSGSETPGTSESATPESSGGSSGGSS | 13,750 | KORV_Q9TTC1-Pro_3mut |
| GGSPAPGGG | 13,751 | MLVMS_P03355_3mutA_WS |
| GGGEAAAKGGS | 13,752 | MLVMS_P03355_3mut |
| GGGEAAAK | 13,753 | PERV_Q4VFZ2_3mut |
| PAPEAAAKGGG | 13,754 | MLVMS_P03355_3mut |
| GSSGSSGSSGSSGSSGSS | 13,755 | BAEVM_P10272_3mutA |
| AEAAAKEAAAKEAAAKEAAAKALEAEAAAKEAAAKEAAAKEAAAKA | 13,756 | GALV_P21414_3mutA |
| EAAAKGGSPAP | 13,757 | FFV_O93209-Pro |
| EAAAKEAAAK | 13,758 | MLVFF_P26809_3mut |
| GGGGSGGGGSGGGGSGGGGSGGGGSGGGGS | 13,759 | PERV_Q4VFZ2_3mutA_WS |
| GGSGGSGGSGGS | 13,760 | MLVAV_P03356_3mutA |
| EAAAKEAAAKEAAAKEAAAKEAAAK | 13,761 | SFV3L_P27401_2mutA |
| GSSGSSGSSGSSGSSGSS | 13,762 | BAEVM_P10272_3mut |
| GGGGS | 13,763 | MLVMS_P03355_PLV919 |
| AEAAAKEAAAKEAAAKEAAAKALEAEAAAKEAAAKEAAAKEAAAKA | 13,764 | SFV1_P23074 |
| GGGGSGGGGS | 13,765 | KORV_Q9TTC1-Pro_3mutA |
| GGGGSGGGGS | 13,766 | MLVMS_P03355_3mut |
| GGSGSS | 13,767 | KORV_Q9TTC1_3mutA |
| GSSPAPGGG | 13,768 | PERV_Q4VFZ2_3mut |
| GSSGGSPAP | 13,769 | PERV_Q4VFZ2_3mutA_WS |
| PAPGGS | 13,770 | PERV_Q4VFZ2_3mutA_WS |
| GGSPAPEAAAK | 13,771 | FOAMV_P14350_2mutA |
| GGGPAPGGS | 13,772 | SFV3L_P27401_2mut |
| PAPGSSGGG | 13,773 | MLVCB_P08361_3mut |
| GSSGGGEAAAK | 13,774 | AVIRE_P03360_3mut |
| GSSGGG | 13,775 | XMRV6_A1Z651_3mut |
| GSSGSS | 13,776 | PERV_Q4VFZ2_3mut |
| GSSGGG | 13,777 | MLVAV_P03356_3mutA |
| PAPGGGGGS | 13,778 | PERV_Q4VFZ2_3mut |
| GSSEAAAK | 13,779 | MLVMS_P03355_3mut |
| PAPGGG | 13,780 | FLV_P10273_3mutA |
| GGGGSGGGGS | 13,781 | PERV_Q4VFZ2_3mut |
| GSSGGS | 13,782 | MLVMS_P03355_PLV919 |
| GGGGSGGGGS | 13,783 | SFV3L_P27401_2mut |
| EAAAKGGSGSS | 13,784 | FLV_P10273_3mutA |
| GSSEAAAKGGS | 13,785 | MLVMS_P03355_3mutA_WS |
| PAPGSSEAAAK | 13,786 | SFV3L_P27401_2mutA |
| GGGGSGGGGS | 13,787 | SFV3L_P27401-Pro_2mutA |
| PAPGSSEAAAK | 13,788 | PERV_Q4VFZ2_3mut |
| PAPGSSEAAAK | 13,789 | PERV_Q4VFZ2 |
| GGSPAPGGG | 13,790 | AVIRE_P03360_3mut |
| GGGGGS | 13,791 | PERV_Q4VFZ2_3mutA_WS |
| GGGGSSGGS | 13,792 | PERV_Q4VFZ2_3mut |
| PAPAPAPAP | 13,793 | AVIRE_P03360_3mutA |
| GGSGGS | 13,794 | WMSV_P03359_3mutA |
| GGGPAPGGS | 13,795 | PERV_Q4VFZ2_3mut |
| GGSGGSGGSGGSGGS | 13,796 | MLVMS_P03355_PLV919 |
| GGSGGG | 13,797 | PERV_Q4VFZ2_3mut |
| EAAAKEAAAK | 13,798 | SFV3L_P27401_2mut |
| PAPGSS | 13,799 | XMRV6_A1Z651_3mut |
| GSSEAAAK | 13,800 | MLVFF_P26809_3mut |
| GGSPAPGGG | 13,801 | MLVMS_P03355_3mut |
| EAAAKGGG | 13,802 | WMSV_P03359_3mutA |
| GSSEAAAKGGS | 13,803 | PERV_Q4VFZ2_3mutA_WS |
| GSSGGSPAP | 13,804 | FFV_O93209 |
| GGGGGS | 13,805 | KORV_Q9TTC1-Pro_3mut |
| GSSGGG | 13,806 | MLVCB_P08361_3mut |
| GSSGSS | 13,807 | MLVCB_P08361_3mutA |
| GGSEAAAKPAP | 13,808 | BAEVM_P10272_3mut |
| EAAAKGGGGSS | 13,809 | MLVCB_P08361_3mut |
| EAAAKPAPGGS | 13,810 | KORV_Q9TTC1-Pro_3mutA |
| GSSGSSGSSGSSGSS | 13,811 | MLVAV_P03356_3mutA |
| GGGGSEAAAKGGGGS | 13,812 | PERV_Q4VFZ2_3mutA_WS |
| GGSGSS | 13,813 | KORV_Q9TTC1-Pro_3mut |
| GSS | | SFV3L_P27401-Pro_2mutA |
| PAPAP | 13,815 | BAEVM_P10272_3mut |
| EAAAKPAP | 13,816 | BAEVM_P10272 |
| EAAAKEAAAKEAAAKEAAAKEAAAK | 13,817 | KORV_Q9TTC1-Pro_3mut |
| GGGGGGG | 13,818 | PERV_Q4VFZ2_3mutA_WS |
| GGGGS | 13,819 | MLVMS_P03355_3mut |
| GSSGGG | 13,820 | FLV_P10273_3mutA |
| PAPAPAPAPAP | 13,821 | FLV_P10273_3mut |
| EAAAKEAAAKEAAAK | 13,822 | WMSV_P03359_3mutA |
| GSSGGS | 13,823 | MLVBM_Q7SVK7_3mutA_WS |
| EAAAKPAPGGG | 13,824 | MLVMS_P03355_3mut |
| GSSPAPGGS | 13,825 | WMSV_P03359_3mut |
| PAPGSSGGG | 13,826 | PERV_Q4VFZ2_3mutA_WS |
| GSSGGG | 13,827 | AVIRE_P03360_3mutA |
| PAPGGSGSS | 13,828 | MLVFF_P26809_3mut |
| PAPGSS | 13,829 | PERV_Q4VFZ2_3mut |
| GGGGGSGSS | 13,830 | WMSV_P03359_3mutA |
| EAAAKGGGGSS | 13,831 | MLVBM_Q7SVK7_3mutA_WS |
| GGGGGGG | 13,832 | BAEVM_P10272_3mut |
| PAPEAAAKGSS | 13,833 | MLVMS_P03355_3mut |
| GGSGGGEAAAK | 13,834 | MLVMS_P03355_PLV919 |
| EAAAKGGGGGS | 13,835 | MLVCB_P08361_3mut |
| PAPGGS | 13,836 | KORV_Q9TTC1-Pro_3mut |
| GGGG | 13,837 | FLV_P10273_3mutA |
| EAAAKGGSGSS | 13,838 | MLVBM_Q7SVK7_3mutA_WS |
| GGGGSSGGS | 13,839 | MLVMS_P03355_3mutA_WS |
| GGGGGGGG | 13,840 | WMSV_P03359_3mut |
| GGSGSSGGG | 13,841 | MLVMS_P03355_PLV919 |
| GSSEAAAKGGS | 13,842 | KORV_Q9TTC1-Pro_3mutA |
| EAAAKPAPGSS | 13,843 | MLVCB_P08361_3mut |
| GGSPAPGSS | 13,844 | KORV_Q9TTC1_3mutA |
| PAPGSSGGG | 13,845 | BAEVM_P10272_3mut |
| EAAAKPAPGSS | 13,846 | WMSV_P03359_3mut |
| GGSPAPEAAAK | 13,847 | XMRV6_A1Z651_3mutA |
| GSSPAP | 13,848 | FLV_P10273_3mutA |
| GSS | | BAEVM_P10272_3mutA |
| EAAAKPAPGGS | 13,850 | FLV_P10273_3mutA |
| GGSGSSPAP | 13,851 | FLV_P10273_3mutA |
| PAPGSSGGS | 13,852 | MLVMS_P03355_3mut |
| GSAGSAAGSGEF | 13,853 | PERV_Q4VFZ2_3mutA_WS |
| GSSGGSEAAAK | 13,854 | KORV_Q9TTC1_3mutA |
| GSSGGS | 13,855 | MLVMS_P03355_3mutA_WS |
| EAAAKGGGGSEAAAK | 13,856 | SFV3L_P27401_2mut |
| GSSGGS | 13,857 | PERV_Q4VFZ2_3mutA_WS |
| GGSPAPEAAAK | 13,858 | FLV_P10273_3mut |
| GGSEAAAKGSS | 13,859 | PERV_Q4VFZ2_3mutA_WS |
| GSSPAPEAAAK | 13,860 | PERV_Q4VFZ2_3mutA_WS |
| GGSGSSGGG | 13,861 | PERV_Q4VFZ2_3mut |
| GGGG | 13,862 | AVIRE_P03360_3mutA |
| GGSEAAAKPAP | 13,863 | WMSV_P03359_3mut |
| GSSGGSPAP | 13,864 | MLVAV_P03356_3mutA |
| GSSGGSEAAAK | 13,865 | MLVMS_P03355_3mut |
| PAPEAAAKGGS | 13,866 | KORV_Q9TTC1-Pro_3mut |
| GGSPAP | 13,867 | PERV_Q4VFZ2_3mutA_WS |
| GGSEAAAK | 13,868 | MLVAV_P03356_3mutA |
| EAAAKGGGGSEAAAK | 13,869 | KORV_Q9TTC1-Pro_3mut |
| SGGSSGGSSGSETPGTSESATPESSGGSSGGSS | 13,870 | MLVMS_P03355_PLV919 |
| GSSEAAAK | 13,871 | KORV_Q9TTC1_3mutA |
| GGG | | AVIRE_P03360 |
| GGSEAAAKGSS | 13,873 | MLVBM_Q7SVK7_3mut |
| GGSEAAAKGSS | 13,874 | MLVMS_P03355_3mut |
| GGSPAPEAAAK | 13,875 | MLVCB_P08361_3mut |
| GGSGGGEAAAK | 13,876 | MLVCB_P08361_3mut |
| GGSEAAAKPAP | 13,877 | MLVMS_P03355_3mutA_WS |
| EAAAKGGSGSS | 13,878 | KORV_Q9TTC1-Pro_3mut |
| GGGEAAAKGGS | 13,879 | MLVCB_P08361_3mut |
| EAAAKGGGGSEAAAK | 13,880 | FLV_P10273_3mutA |
| GGSPAP | 13,881 | MLVFF_P26809_3mut |
| GGGGSSGGS | 13,882 | XMRV6_A1Z651_3mutA |
| PAP | | MLVCB_P08361_3mut |
| GGS | | SFV3L_P27401-Pro_2mutA |
| GGGGSGGGGS | 13,885 | MLVMS_P03355_3mut |
| GGGEAAAKGGS | 13,886 | MLVAV_P03356_3mutA |
| GSSGSSGSSGSSGSSGSS | 13,887 | MLVMS_P03355_PLV919 |
| PAPGSS | 13,888 | MLVCB_P08361_3mut |
| GGSGGSGGS | 13,889 | MLVMS_P03355_PLV919 |
| PAPGGSGGG | 13,890 | FLV_P10273_3mutA |
| GGGGSGGGGSGGGGS | 13,891 | FLV_P10273_3mut |
| GGSGSSGGG | 13,892 | KORV_Q9TTC1-Pro_3mutA |
| GGSGGSGGS | 13,893 | GALV_P21414_3mutA |
| GGGEAAAKGGS | 13,894 | WMSV_P03359_3mut |
| SGSETPGTSESATPES | 13,895 | KORV_Q9TTC1_3mutA |
| EAAAKGGGGGS | 13,896 | KORV_Q9TTC1-Pro_3mut |
| EAAAKGSSPAP | 13,897 | BAEVM_P10272_3mut |
| GGGG | 13,898 | MLVCB_P08361_3mut |
| GGGGSGGGGSGGGGSGGGGSGGGGS | 13,899 | MLVBM_Q7SVK7_3mut |
| GSSGGSGGG | 13,900 | MLVMS_P03355_PLV919 |
| GGSGSS | 13,901 | MLVFF_P26809_3mut |
| EAAAKGGS | 13,902 | AVIRE_P03360_3mutA |
| GSSEAAAKGGS | 13,903 | MLVBM_Q7SVK7_3mutA_WS |
| EAAAKPAPGGG | 13,904 | WMSV_P03359_3mut |
| PAPGSSGGG | 13,905 | MLVCB_P08361_3mutA |
| GGGGSSEAAAK | 13,906 | KORV_Q9TTC1-Pro_3mutA |
| GSSEAAAKPAP | 13,907 | BAEVM_P10272_3mutA |
| PAPGGGEAAAK | 13,908 | MLVBM_Q7SVK7_3mutA_WS |
| GGSGGGEAAAK | 13,909 | MLVCB_P08361_3mutA |
| GGGGGGGGSGGGGSGGGGSGGGGSGGGGS | 13,910 | FFV_O93209 |
| EAAAKGGGGGS | 13,911 | GALV_P21414_3mutA |
| GGSPAPGGG | 13,912 | MLVMS_P03355_3mut |
| GSSGSSGSS | 13,913 | FLV_P10273_3mutA |
| EAAAK | 13,914 | MLVBM_Q7SVK7_3mut |
| GGGGSSGGS | 13,915 | MLVMS_P03355_3mut |
| GGSGSSPAP | 13,916 | PERV_Q4VFZ2_3mut |
| EAAAKEAAAKEAAAKEAAAK | 13,917 | BAEVM_P10272_3mut |
| GGGPAPGSS | 13,918 | MLVMS_P03355_3mut |
| GSSPAPGGS | 13,919 | PERV_Q4VFZ2_3mutA_WS |
| PAPAP | 13,920 | FLV_P10273_3mutA |
| PAPAPAPAP | 13,921 | PERV_Q4VFZ2_3mut |
| GGGGGSEAAAK | 13,922 | GALV_P21414_3mutA |
| GGGGGSGSS | 13,923 | BAEVM_P10272_3mutA |
| GGGEAAAKGSS | 13,924 | KORV_Q9TTC1_3mutA |
| GGGGGSPAP | 13,925 | AVIRE_P03360_3mut |
| GGGGGSEAAAK | 13,926 | SFV3L_P27401_2mutA |
| GGS | | KORV_Q9TTC1_3mutA |
| GGGGGGG | 13,928 | PERV_Q4VFZ2_3mut |
| SGSETPGTSESATPES | 13,929 | SFV3L_P27401_2mutA |
| EAAAKGGSGGG | 13,930 | MLVMS_P03355_3mut |
| GGGGS | 13,931 | MLVFF_P26809_3mut |
| EAAAKGSSGGG | 13,932 | BAEVM_P10272_3mut |
| EAAAKPAPGGS | 13,933 | MLVF5_P26810_3mutA |
| SGGSSGGSSGSETPGTSESATPESSGGSSGGSS | 13,934 | SFV3L_P27401_2mutA |
| GGSPAPGGG | 13,935 | WMSV_P03359_3mutA |
| GSAGSAAGSGEF | 13,936 | MLVFF_P26809_3mut |
| GGGGSSGGS | 13,937 | MLVMS_P03355_3mutA_WS |
| GGGGGGG | 13,938 | MLVCB_P08361_3mut |
| GSSEAAAK | 13,939 | WMSV_P03359_3mut |
| PAPGSS | 13,940 | FLV_P10273_3mutA |
| GSSGGG | 13,941 | PERV_Q4VFZ2_3mutA_WS |
| PAPGGG | 13,942 | MLVFF_P26809_3mut |
| GGGGGSPAP | 13,943 | MLVMS_P03355_3mut |
| GGSEAAAK | 13,944 | XMRV6_A1Z651_3mut |
| GSSGGG | 13,945 | PERV_Q4VFZ2_3mut |
| GGSGGSGGSGGS | 13,946 | MLVMS_P03355_3mut |
| PAPAP | 13,947 | AVIRE_P03360_3mut |
| GGSEAAAK | 13,948 | PERV_Q4VFZ2_3mut |
| GGGGS | 13,949 | MLVMS_P03355_PLV919 |
| GGGG | 13,950 | BAEVM_P10272_3mutA |
| EAAAKGGGGSS | 13,951 | MLVCB_P08361_3mutA |
| EAAAKEAAAKEAAAK | 13,952 | GALV_P21414_3mutA |
| PAPGGGEAAAK | 13,953 | KORV_Q9TTC1 |
| EAAAKGGSPAP | 13,954 | MLVMS_P03355_3mut |
| GGSGSSEAAAK | 13,955 | MLVMS_P03355_3mut |
| GGSPAPEAAAK | 13,956 | FLV_P10273_3mutA |
| GGGGGGG | 13,957 | PERV_Q4VFZ2_3mut |
| EAAAKEAAAKEAAAKEAAAKEAAAKEAAAK | 13,958 | SFV1_P23074_2mutA |
| EAAAKGSSGGS | 13,959 | MLVMS_P03355_3mut |
| GSSEAAAKPAP | 13,960 | MLVFF_P26809_3mut |
| GGGGSS | 13,961 | FLV_P10273_3mutA |
| EAAAKGGSGGG | 13,962 | AVIRE_P03360_3mutA |
| GGSGGS | 13,963 | PERV_Q4VFZ2_3mutA_WS |
| GGGGGSPAP | 13,964 | AVIRE_P03360_3mutA |
| EAAAKEAAAKEAAAK | 13,965 | XMRV6_A1Z651_3mut |
| PAPEAAAKGGS | 13,966 | FLV_P10273_3mutA |
| GSSGGSEAAAK | 13,967 | MLVCB_P08361_3mut |
| EAAAKGGSGGG | 13,968 | MLVMS_P03355 |
| GGSGGGPAP | 13,969 | MLVMS_P03355_3mut |
| GGS | | XMRV6_A1Z651_3mut |
| GGSEAAAKPAP | 13,971 | MLVFF_P26809_3mut |
| EAAAKGGG | 13,972 | MLVMS_P03355_PLV919 |
| GSSGSSGSSGSS | 13,973 | WMSV_P03359_3mut |
| GGSGSSPAP | 13,974 | PERV_Q4VFZ2_3mut |
| GGGEAAAK | 13,975 | MLVMS_P03355_3mutA_WS |
| GSSPAPGGS | 13,976 | KORV_Q9TTC1-Pro_3mutA |
| GSSEAAAKGGG | 13,977 | SFV3L_P27401_2mut |
| EAAAKPAPGGS | 13,978 | MLVCB_P08361_3mut |
| GGSGGGEAAAK | 13,979 | PERV_Q4VFZ2 |
| GGSGSS | 13,980 | MLVCB_P08361_3mut |
| GGSGGGEAAAK | 13,981 | MLVBM_Q7SVK7_3mutA_WS |
| GGSGGSGGSGGSGGSGGS | 13,982 | FLV_P10273_3mut |
| PAPEAAAKGSS | 13,983 | MLVMS_P03355_3mut |
| EAAAKGSSGGS | 13,984 | WMSV_P03359_3mutA |
| GGSGSSEAAAK | 13,985 | MLVCB_P08361_3mut |
| GGSGSSEAAAK | 13,986 | KORV_Q9TTC1_3mutA |
| GSSGGSGGG | 13,987 | MLVMS_P03355_PLV919 |
| EAAAKGGSGGG | 13,988 | SFV3L_P27401-Pro_2mutA |
| GGSGGS | 13,989 | AVIRE_P03360_3mutA |
| GSAGSAAGSGEF | 13,990 | MLVMS_P03355_PLV919 |
| GGSGSS | 13,991 | GALV_P21414_3mutA |
| GGGG | 13,992 | MLVFF_P26809_3mutA |
| GGGGSGGGGSGGGGSGGGGS | 13,993 | WMSV_P03359_3mut |
| SGSETPGTSESATPES | 13,994 | BAEVM_P10272_3mut |
| EAAAKEAAAKEAAAKEAAAK | 13,995 | FOAMV_P14350_2mutA |
| GGGEAAAKGGS | 13,996 | FLV_P10273_3mutA |
| GSSGGSEAAAK | 13,997 | MLVFF_P26809_3mut |
| EAAAKGGGGSS | 13,998 | MLVAV_P03356_3mut |
| PAPGGSEAAAK | 13,999 | KORV_Q9TTC1-Pro_3mut |
| EAAAK | 14,000 | XMRV6_A1Z651_3mut |
| GSSGSSGSSGSSGSSGSS | 14,001 | PERV_Q4VFZ2_3mut |
| GGGG | 14,002 | MLVCB_P08361_3mutA |
| GSSGSS | 14,003 | WMSV_P03359_3mutA |
| GSSGGSPAP | 14,004 | AVIRE_P03360_3mut |
| GGSGGSGGS | 14,005 | MLVCB_P08361_3mut |
| EAAAKGGGPAP | 14,006 | FLV_P10273_3mutA |
| GGGGSGGGGS | 14,007 | MLVCB_P08361_3mut |
| GGSEAAAKGSS | 14,008 | PERV_Q4VFZ2_3mutA_WS |
| EAAAKEAAAKEAAAKEAAAKEAAAKEAAAK | 14,009 | SFV3L_P27401_2mutA |
| GGSGSSEAAAK | 14,010 | PERV_Q4VFZ2_3mutA_WS |
| EAAAKEAAAKEAAAKEAAAK | 14,011 | SFV3L_P27401-Pro_2mutA |
| GSSEAAAKGGS | 14,012 | FLV_P10273_3mutA |
| GGSGSS | 14,013 | PERV_Q4VFZ2 |
| GGSGSSEAAAK | 14,014 | SFV3L_P27401-Pro_2mutA |
| GSSGSSGSS | 14,015 | XMRV6_A1Z651_3mutA |
| EAAAKGSSPAP | 14,016 | KORV_Q9TTC1_3mutA |
| EAAAKPAP | 14,017 | FLV_P10273_3mutA |
| GGSGSSEAAAK | 14,018 | KORV_Q9TTC1-Pro_3mut |
| GGGGSGGGGSGGGGSGGGGSGGGGSGGGGS | 14,019 | KORV_Q9TTC1_3mutA |
| GGGGSGGGGSGGGGS | 14,020 | KORV_Q9TTC1-Pro_3mutA |
| GGGGGGG | 14,021 | FLV_P10273_3mut |
| EAAAKGSS | 14,022 | WMSV_P03359_3mut |
| EAAAKGGGPAP | 14,023 | MLVCB_P08361_3mut |
| GSSGSS | 14,024 | MLVBM_Q7SVK7_3mutA_WS |
| EAAAKGGGGGS | 14,025 | MLVFF_P26809_3mut |
| GGSGGGEAAAK | 14,026 | FLV_P10273_3mutA |
| PAPGSS | 14,027 | MLVFF_P26809_3mutA |
| PAPGSS | 14,028 | BAEVM_P10272_3mutA |
| GGSPAPGSS | 14,029 | AVIRE_P03360_3mut |
| GGGGSSEAAAK | 14,030 | MLVMS_P03355_3mut |
| GSSGGGGGS | 14,031 | FFV_O93209-Pro |
| EAAAKGSSPAP | 14,032 | PERV_Q4VFZ2_3mut |
| GSSPAPGGS | 14,033 | PERV_Q4VFZ2_3mut |
| GGGGGG | 14,034 | BAEVM_P10272_3mut |
| EAAAKGGGGSS | 14,035 | PERV_Q4VFZ2_3mutA_WS |
| PAPGGSEAAAK | 14,036 | KORV_Q9TTC1_3mutA |
| SGGSSGGSSGSETPGTSESATPESSGGSSGGSS | 14,037 | MLVMS_P03355_3mutA_WS |
| GSSGSSGSSGSS | 14,038 | MLVMS_P03355_3mut |
| EAAAKGSSGGG | 14,039 | MLVMS_P03355_PLV919 |
| GGSEAAAKPAP | 14,040 | AVIRE_P03360_3mutA |
| GSSGSSGSSGSSGSS | 14,041 | WMSV_P03359_3mutA |
| GGGEAAAKPAP | 14,042 | FLV_P10273_3mutA |
| PAPGSSGGG | 14,043 | KORV_Q9TTC1_3mutA |
| GSSGSS | 14,044 | MLVMS_P03355_3mutA_WS |
| PAPEAAAK | 14,045 | BAEVM_P10272_3mut |
| GGGPAPGSS | 14,046 | PERV_Q4VFZ2 |
| GSSGGSPAP | 14,047 | MLVFF_P26809_3mut |
| GGGGSS | 14,048 | SFV3L_P27401_2mut |
| PAPEAAAKGSS | 14,049 | SFV3L_P27401_2mut |
| GGSGGGPAP | 14,050 | XMRV6_A1Z651_3mutA |
| PAPGGS | 14,051 | BAEVM_P10272_3mutA |
| EAAAKGGGGGS | 14,052 | AVIRE_P03360_3mut |
| GSSGGSPAP | 14,053 | KORV_Q9TTC1-Pro_3mutA |
| GSSGGGGGS | 14,054 | WMSV_P03359_3mut |
| GGGEAAAKGGS | 14,055 | AVIRE_P03360_3mut |
| GGGEAAAKGSS | 14,056 | BAEVM_P10272_3mut |
| PAPEAAAKGSS | 14,057 | MLVAV_P03356_3mutA |
| GSSGSSGSSGSSGSS | 14,058 | MLVCB_P08361_3mut |
| GGSPAPGSS | 14,059 | FLV_P10273_3mutA |
| EAAAKGSSPAP | 14,060 | BAEVM_P10272_3mutA |
| GGSGGSGGSGGSGGSGGS | 14,061 | PERV_Q4VFZ2 |
| GGGGSSEAAAK | 14,062 | FLV_P10273_3mutA |
| GGGGSSPAP | 14,063 | FFV_093209 |
| GSSGGSPAP | 14,064 | MLVMS_P03355_3mut |
| GGGPAPGSS | 14,065 | MLVMS_P03355_PLV919 |
| PAPGSSGGS | 14,066 | PERV_Q4VFZ2_3mut |
| GGGGGSPAP | 14,067 | MLVFF_P26809_3mut |
| SGSETPGTSESATPES | 14,068 | MLVMS_P03355_3mutA_WS |
| GSSGSSGSSGSSGSS | 14,069 | KORV_Q9TTC1_3mutA |
| GSSPAPGGG | 14,070 | WMSV_P03359_3mut |
| PAPAPAPAPAPAP | 14,071 | SFV3L_P27401_2mutA |
| GGGPAPGGS | 14,072 | MLVMS_P03355_3mut |
| PAPGGSEAAAK | 14,073 | WMSV_P03359_3mut |
| GGGGSSEAAAK | 14,074 | FFV_O93209-Pro |
| GGSPAPGGG | 14,075 | FLV_P10273_3mutA |
| GSSPAPEAAAK | 14,076 | AVIRE_P03360_3mut |
| GGGEAAAK | 14,077 | FLV_P10273_3mutA |
| PAPEAAAKGGG | 14,078 | MLVCB_P08361_3mut |
| GGSPAPGGG | 14,079 | MLVCB_P08361_3mut |
| GGSGGGGSS | 14,080 | BAEVM_P10272_3mutA |
| GSSPAPEAAAK | 14,081 | MLVCB_P08361_3mut |
| GGSPAPGGG | 14,082 | KORV_Q9TTC1-Pro_3mutA |
| PAPGGSGSS | 14,083 | KORV_Q9TTC1_3mutA |
| GSSPAP | 14,084 | KORV_Q9TTC1-Pro_3mutA |
| SGSETPGTSESATPES | 14,085 | MLVMS_P03355 |
| GSSGSSGSS | 14,086 | MLVAV_P03356_3mutA |
| PAPGSSGGS | 14,087 | PERV_Q4VFZ2_3mutA_WS |
| PAPGGS | 14,088 | KORV_Q9TTC1-Pro_3mutA |
| PAPEAAAKGGG | 14,089 | SFV3L_P27401-Pro_2mutA |
| GGSGGSGGS | 14,090 | BAEVM_P10272_3mut |
| PAPGGS | 14,091 | MLVFF_P26809_3mut |
| GSSGGSPAP | 14,092 | MLVMS_P03355_PLV919 |
| GSSGGGGGS | 14,093 | FLV_P10273_3mutA |
| GGGGGSPAP | 14,094 | KORV_Q9TTC1-Pro_3mut |
| EAAAKPAPGSS | 14,095 | SFV3L_P27401-Pro_2mutA |
| EAAAKGGSPAP | 14,096 | KORV_Q9TTC1-Pro |
| GGGPAPEAAAK | 14,097 | MLVMS_P03355_PLV919 |
| GGSEAAAKGSS | 14,098 | MLVMS_P03355 |
| PAPEAAAKGSS | 14,099 | KORV_Q9TTC1_3mutA |
| PAPEAAAKGGS | 14,100 | WMSV_P03359_3mutA |
| GSSGGG | 14,101 | PERV_Q4VFZ2_3mutA_WS |
| EAAAKGGGGSS | 14,102 | MLVMS_P03355_PLV919 |
| EAAAKGGSPAP | 14,103 | AVIRE_P03360_3mutA |
| GGGGSSGGS | 14,104 | MLVMS_P03355_PLV919 |
| PAPEAAAKGSS | 14,105 | PERV_Q4VFZ2_3mutA_WS |
| EAAAKGGGGGS | 14,106 | BAEVM_P10272_3mut |
| GSSGGGGGS | 14,107 | MLVMS_P03355_3mut |
| PAPAPAPAP | 14,108 | KORV_Q9TTC1_3mutA |
| GGSGGSGGSGGS | 14,109 | MLVAV_P03356_3mut |
| PAPAPAPAP | 14,110 | SFV3L_P27401_2mut |
| GSSEAAAKPAP | 14,111 | MLVMS_P03355_3mut |
| GGSGGGEAAAK | 14,112 | SFV3L_P27401_2mutA |
| GSSGGSGGG | 14,113 | MLVMS_P03355_3mutA_WS |
| GGGGGSPAP | 14,114 | MLVCB_P08361_3mutA |
| GGGEAAAKGSS | 14,115 | XMRV6_A1Z651_3mutA |
| GGGGSSPAP | 14,116 | BAEVM_P10272_3mut |
| GGSGGG | 14,117 | PERV_Q4VFZ2_3mut |
| GGGGSS | 14,118 | MLVBM_Q7SVK7_3mutA_WS |
| EAAAKGSSGGS | 14,119 | PERV_Q4VFZ2_3mutA_WS |
| GSSGGGGGS | 14,120 | PERV_Q4VFZ2 |
| EAAAKGSSGGS | 14,121 | PERV_Q4VFZ2_3mut |
| EAAAKEAAAK | 14,122 | MLVAV_P03356_3mut |
| GSSGGGEAAAK | 14,123 | MLVAV_P03356_3mut |
| GSSPAPGGG | 14,124 | XMRV6_A1Z651_3mut |
| GGGGSGGGGSGGGGS | 14,125 | PERV_Q4VFZ2_3mut |
| EAAAKEAAAKEAAAKEAAAK | 14,126 | KORV_Q9TTC1_3mutA |
| EAAAKGGSGSS | 14,127 | MLVBM_Q7SVK7_3mut |
| PAPEAAAK | 14,128 | BLVJ_P03361 |
| GSSGGG | 14,129 | FFV_O93209-Pro |
| GGSGGGEAAAK | 14,130 | KORV_Q9TTC1-Pro_3mutA |
| EAAAK | 14,131 | FLV_P10273_3mutA |
| GGGGSSPAP | 14,132 | MLVMS_P03355_3mut |
| GSS | | SFV3L_P27401-Pro_2mut |
| PAPEAAAKGSS | 14,134 | BAEVM_P10272_3mut |
| GGGGGSPAP | 14,135 | PERV_Q4VFZ2_3mut |
| GSSGSSGSS | 14,136 | BAEVM_P10272_3mutA |
| GGGGSGGGGSGGGGSGGGGS | 14,137 | SFV1_P23074_2mut |
| GGGGSSEAAAK | 14,138 | SFV3L_P27401_2mutA |
| GGGGSGGGGSGGGGSGGGGS | 14,139 | FOAMV_P14350-Pro_2mut |
| PAPGSSEAAAK | 14,140 | MLVBM_Q7SVK7_3mutA_WS |
| GGGGGSGSS | 14,141 | MLVFF_P26809_3mutA |
| GGSEAAAKGGG | 14,142 | MLVBM_Q7SVK7_3mut |
| PAPGSSGGG | 14,143 | PERV_Q4VFZ2 |
| GGS | | PERV_Q4VFZ2_3mutA_WS |
| EAAAKGGSGSS | 14,145 | FLV_P10273_3mut |
| GGGEAAAK | 14,146 | WMSV_P03359_3mutA |
| GGSEAAAKPAP | 14,147 | MLVBM_Q7SVK7_3mut |
| SGSETPGTSESATPES | 14,148 | FOAMV_P14350-Pro_2mutA |
| EAAAKPAPGGS | 14,149 | AVIRE_P03360_3mut |
| EAAAKGGGGGS | 14,150 | KORV_Q9TTC1-Pro_3mutA |
| GGGGS | 14,151 | PERV_Q4VFZ2_3mut |
| GGSEAAAKGSS | 14,152 | MLVFF_P26809_3mutA |
| GGSEAAAKGGG | 14,153 | AVIRE_P03360 |
| GGSGGSGGSGGSGGSGGS | 14,154 | SFV3L_P27401_2mut |
| GGSEAAAKGSS | 14,155 | SFV3L_P27401-Pro_2mutA |
| GGGEAAAKPAP | 14,156 | MLVCB_P08361_3mut |
| GGSEAAAK | 14,157 | MLVMS_P03355_PLV919 |
| GGSPAPGSS | 14,158 | KORV_Q9TTC1-Pro_3mutA |
| GSSPAPEAAAK | 14,159 | WMSV_P03359_3mutA |
| GGSGSS | 14,160 | KORV_Q9TTC1-Pro_3mutA |
| PAPGGGGGS | 14,161 | AVIRE_P03360_3mut |
| PAPEAAAKGSS | 14,162 | FFV_O93209-Pro |
| GGSGGGEAAAK | 14,163 | WMSV_P03359_3mut |
| PAPGGG | 14,164 | MLVMS_P03355_3mut |
| EAAAKGGG | 14,165 | FLV_P10273_3mutA |
| GSSGSSGSSGSS | 14,166 | MLVCB_P08361_3mut |
| EAAAKGGSGGG | 14,167 | FFV_093209 |
| GSSPAPGGS | 14,168 | PERV_Q4VFZ2_3mutA_WS |
| GSSPAPGGS | 14,169 | MLVCB_P08361_3mut |
| GGGPAP | 14,170 | WMSV_P03359_3mutA |
| GGGPAP | 14,171 | KORV_Q9TTC1_3mutA |
| GGSPAPGSS | 14,172 | KORV_Q9TTC1-Pro_3mut |
| PAPAP | 14,173 | MLVMS_P03355_3mut |
| GGGGGGG | 14,174 | MLVMS_P03355_3mut |
| GGGGG | 14,175 | KORV_Q9TTC1-Pro_3mut |
| GSAGSAAGSGEF | 14,176 | FOAMV_P14350_2mutA |
| PAPAP | 14,177 | KORV_Q9TTC1-Pro_3mutA |
| GGSEAAAKGGG | 14,178 | SFV3L_P27401-Pro_2mutA |
| PAPAP | 14,179 | WMSV_P03359_3mut |
| GGGGSGGGGSGGGGS | 14,180 | SFV3L_P27401_2mut |
| PAPGGS | 14,181 | KORV_Q9TTC1_3mutA |
| GGGEAAAKPAP | 14,182 | FLV_P10273_3mut |
| GGGGGS | 14,183 | MLVAV_P03356_3mutA |
| GSSEAAAKGGG | 14,184 | WMSV_P03359_3mut |
| EAAAKGGGGSS | 14,185 | GALV_P21414_3mutA |
| GSSGGS | 14,186 | MLVAV_P03356_3mutA |
| GSSGGG | 14,187 | MLVBM_Q7SVK7_3mut |
| PAPAPAP | 14,188 | SFV3L_P27401-Pro_2mutA |
| GGGG | 14,189 | KORV_Q9TTC1_3mutA |
| EAAAKPAPGGS | 14,190 | MLVFF_P26809_3mut |
| GGGGSGGGGS | 14,191 | XMRV6_A1Z651_3mut |
| EAAAKGGG | 14,192 | MLVCB_P08361_3mut |
| GGGGSSPAP | 14,193 | KORV_Q9TTC1_3mutA |
| GSSEAAAKGGG | 14,194 | KORV_Q9TTC1-Pro_3mutA |
| GGGGG | 14,195 | BLVJ_P03361_2mutB |
| GGGEAAAKGSS | 14,196 | FFV_O93209-Pro |
| GSSGSSGSS | 14,197 | BAEVM_P10272_3mut |
| GSSGGSPAP | 14,198 | PERV_Q4VFZ2_3mut |
| EAAAKGGS | 14,199 | KORV_Q9TTC1_3mut |
| GGSPAPEAAAK | 14,200 | AVIRE_P03360_3mut |
| GGSEAAAK | 14,201 | WMSV_P03359_3mut |
| GSSGGS | 14,202 | KORV_Q9TTC1-Pro_3mutA |
| GGGPAPEAAAK | 14,203 | KORV_Q9TTC1_3mutA |
| PAPGSS | 14,204 | WMSV_P03359_3mutA |
| GGSEAAAKGSS | 14,205 | FLV_P10273_3mutA |
| EAAAKEAAAKEAAAKEAAAKEAAAK | 14,206 | SFV3L_P27401 |
| GSSEAAAKGGG | 14,207 | SFV3L_P27401-Pro_2mutA |
| GGGGSEAAAKGGGGS | 14,208 | KORV_Q9TTC1-Pro_3mutA |
| GGSGGSGGS | 14,209 | WMSV_P03359_3mut |
| GGGGGSGSS | 14,210 | KORV_Q9TTC1-Pro |
| GGGGSGGGGSGGGGSGGGGS | 14,211 | MLVMS_P03355_3mut |
| EAAAKGGG | 14,212 | PERV_Q4VFZ2 |
| GGSEAAAKGGG | 14,213 | KORV_Q9TTC1-Pro_3mut |
| GSSGGSGGG | 14,214 | PERV_Q4VFZ2_3mutA_WS |
| GGGGGS | 14,215 | PERV_Q4VFZ2_3mut |
| GSAGSAAGSGEF | 14,216 | PERV_Q4VFZ2 |
| PAPEAAAKGSS | 14,217 | BAEVM_P10272_3mutA |
| GSSPAPGGG | 14,218 | MLVCB_P08361_3mut |
| GGGGSSPAP | 14,219 | KORV_Q9TTC1-Pro_3mutA |
| PAPGGSGGG | 14,220 | MLVFF_P26809_3mut |
| GSSPAP | 14,221 | KORV_Q9TTC1_3mutA |
| PAPGSS | 14,222 | SFV3L_P27401-Pro_2mut |
| GGSGGGGSS | 14,223 | MLVMS_P03355_PLV919 |
| GSSGGS | 14,224 | WMSV_P03359_3mutA |
| EAAAKGGGGGS | 14,225 | PERV_Q4VFZ2 |
| GGGGG | 14,226 | KORV_Q9TTC1_3mutA |
| EAAAKGSS | 14,227 | MLVMS_P03355_PLV919 |
| EAAAKEAAAKEAAAKEAAAKEAAAK | 14,228 | FLV_P10273_3mut |
| EAAAKEAAAKEAAAKEAAAK | 14,229 | SFV3L_P27401-Pro_2mut |
| GSAGSAAGSGEF | 14,230 | SFV3L_P27401_2mutA |
| GGGPAPGGS | 14,231 | FLV_P10273_3mutA |
| GGSEAAAKGGG | 14,232 | MLVCB_P08361_3mut |
| PAPGGGEAAAK | 14,233 | BAEVM_P10272_3mut |
| EAAAKPAPGSS | 14,234 | FOAMV_P14350_2mut |
| GGSEAAAK | 14,235 | KORV_Q9TTC1_3mutA |
| GGSGSS | 14,236 | AVIRE_P03360 |
| GGSPAPEAAAK | 14,237 | MLVMS_P03355_PLV919 |
| GGGGS | 14,238 | XMRV6_A1Z651_3mut |
| GGSPAPGGG | 14,239 | XMRV6_A1Z651_3mut |
| EAAAKPAPGGS | 14,240 | PERV_Q4VFZ2 |
| GSSPAP | 14,241 | BAEVM_P10272_3mut |
| GGSGSSGGG | 14,242 | FLV_P10273_3mutA |
| PAPGGG | 14,243 | PERV_Q4VFZ2_3mutA_WS |
| GSSGGSEAAAK | 14,244 | MLVBM_Q7SVK7_3mut |
| GGSEAAAK | 14,245 | MLVMS_P03355_3mut |
| GGGPAPGGS | 14,246 | MLVFF_P26809_3mut |
| GSAGSAAGSGEF | 14,247 | MLVBM_Q7SVK7_3mutA_WS |
| EAAAKPAPGGS | 14,248 | SFVCP_Q87040 |
| PAPGGG | 14,249 | PERV_Q4VFZ2_3mutA_WS |
| GSSPAPEAAAK | 14,250 | MLVBM_Q7SVK7 |
| PAPEAAAK | 14,251 | MLVBM_Q7SVK7_3mut |
| PAPGGGGGS | 14,252 | AVIRE_P03360_3mutA |
| GGSEAAAKPAP | 14,253 | MLVBM_Q7SVK7_3mut |
| EAAAKGSS | 14,254 | WMSV_P03359_3mutA |
| GGGEAAAK | 14,255 | MLVFF_P26809_3mutA |
| EAAAKEAAAKEAAAK | 14,256 | MLVMS_P03355_3mut |
| PAPEAAAKGGG | 14,257 | BAEVM_P10272_3mut |
| PAPAPAP | 14,258 | MLVCB_P08361_3mut |
| EAAAKPAPGGS | 14,259 | BAEVM_P10272_3mut |
| GGGGSGGGGS | 14,260 | FLV_P10273_3mut |
| GGGGSEAAAKGGGGS | 14,261 | KORV_Q9TTC1_3mut |
| EAAAK | 14,262 | FLV_P10273_3mut |
| PAPAPAP | 14,263 | WMSV_P03359_3mut |
| GGGGSEAAAKGGGGS | 14,264 | FFV_O93209-Pro |
| GGSPAPEAAAK | 14,265 | MLVMS_P03355_3mut |
| GGSGSSGGG | 14,266 | XMRV6_A1Z651_3mut |
| GGSPAPGSS | 14,267 | PERV_Q4VFZ2_3mut |
| SGGSSGGSSGSETPGTSESATPESSGGSSGGSS | 14,268 | SFV3L_P27401-Pro_2mutA |
| EAAAKGGGPAP | 14,269 | BAEVM_P10272_3mutA |
| GSSGGSEAAAK | 14,270 | MLVMS_P03355_3mutA_WS |
| SGSETPGTSESATPES | 14,271 | PERV_Q4VFZ2_3mutA_WS |
| EAAAKEAAAKEAAAKEAAAKEAAAK | 14,272 | KORV_Q9TTC1-Pro_3mutA |
| GSSGSSGSS | 14,273 | KORV_Q9TTC1_3mutA |
| GSSPAPGGG | 14,274 | SFV3L_P27401-Pro_2mutA |
| GSSGGGEAAAK | 14,275 | KORV_Q9TTC1_3mutA |
| GGSGGGGSS | 14,276 | PERV_Q4VFZ2_3mutA_WS |
| GSSGGGEAAAK | 14,277 | MLVCB_P08361_3mut |
| GSSEAAAKGGG | 14,278 | MLVCB_P08361_3mut |
| GGSGGGGSS | 14,279 | KORV_Q9TTC1_3mutA |
| GGSGSSPAP | 14,280 | PERV_Q4VFZ2_3mutA_WS |
| GSSPAP | 14,281 | MLVMS_P03355_3mut |
| GGGGSSEAAAK | 14,282 | AVIRE_P03360 |
| GGS | | WMSV_P03359_3mut |
| EAAAKEAAAK | 14,284 | PERV_Q4VFZ2_3mut |
| PAPAPAPAP | 14,285 | MLVAV_P03356_3mut |
| GGSEAAAKGGG | 14,286 | KORV_Q9TTC1_3mutA |
| PAPGGG | 14,287 | MLVAV_P03356_3mut |
| EAAAKGSS | 14,288 | BAEVM_P10272_3mut |
| GGGGSGGGGS | 14,289 | WMSV_P03359_3mutA |
| GGSGGSGGS | 14,290 | SFV3L_P27401_2mut |
| EAAAK | 14,291 | MLVCB_P08361_3mut |
| GGGGSSGGS | 14,292 | WMSV_P03359_3mutA |
| GGGPAPEAAAK | 14,293 | MLVAV_P03356_3mutA |
| EAAAKEAAAKEAAAK | 14,294 | FFV_093209 |
| GSSEAAAKGGG | 14,295 | MLVBM_Q7SVK7_3mut |
| GGGPAPGGS | 14,296 | FLV_P10273_3mut |
| GGSEAAAKGGG | 14,297 | WMSV_P03359_3mut |
| EAAAKGGGGGS | 14,298 | XMRV6_A1Z651_3mutA |
| EAAAKGGSGGG | 14,299 | FLV_P10273_3mutA |
| GGSEAAAKGGG | 14,300 | SFV3L_P27401_2mutA |
| GGGGS | 14,301 | PERV_Q4VFZ2_3mutA_WS |
| GSSGGS | 14,302 | MLVMS_P03355_3mut |
| GSSGSS | 14,303 | MLVAV_P03356_3mutA |
| GGSPAPGGG | 14,304 | MLVBM_Q7SVK7_3mutA_WS |
| GSSGGGGGS | 14,305 | MLVF5_P26810_3mut |
| PAPAPAPAP | 14,306 | MLVCB_P08361_3mut |
| PAPAP | 14,307 | PERV_Q4VFZ2_3mutA_WS |
| PAPGSSGGS | 14,308 | KORV_Q9TTC1_3mut |
| PAPGSSGGG | 14,309 | PERV_Q4VFZ2_3mut |
| GGGEAAAK | 14,310 | MLVMS_P03355_PLV919 |
| GGSGGSGGSGGSGGS | 14,311 | SFV3L_P27401-Pro_2mutA |
| GGSGGG | 14,312 | FLV_P10273_3mut |
| PAPEAAAKGGG | 14,313 | MLVFF_P26809_3mut |
| PAP | | PERV_Q4VFZ2_3mutA_WS |
| PAPGGSGSS | 14,315 | FFV_093209_2mut |
| EAAAKEAAAKEAAAKEAAAKEAAAKEAAAK | 14,316 | FFV_O93209-Pro_2mut |
| GSSGSSGSSGSS | 14,317 | FFV_093209-Pro |
| GSSGSSGSSGSSGSS | 14,318 | FLV_P10273_3mutA |
| GGGEAAAKPAP | 14,319 | PERV_Q4VFZ2 |
| PAPGSSGGG | 14,320 | SFV3L_P27401_2mut |
| PAPGGSGSS | 14,321 | KORV_Q9TTC1-Pro_3mut |
| PAPAPAPAPAP | 14,322 | GALV_P21414_3mutA |
| GGSGGGEAAAK | 14,323 | PERV_Q4VFZ2_3mut |
| GSSPAP | 14,324 | MLVCB_P08361_3mut |
| EAAAKPAP | 14,325 | MLVF5_P26810_3mut |
| GGGGSGGGGSGGGGSGGGGS | 14,326 | MLVBM_Q7SVK7_3mut |
| GGSGGG | 14,327 | WMSV_P03359_3mut |
| GGSGGSGGS | 14,328 | KORV_Q9TTC1_3mut |
| GGGGGGGG | 14,329 | MLVFF_P26809_3mut |
| GGGGSS | 14,330 | MLVAV_P03356_3mut |
| GSSGGGGGS | 14,331 | SFV3L_P27401_2mut |
| EAAAKEAAAKEAAAKEAAAKEAAAKEAAAK | 14,332 | GALV_P21414_3mutA |
| GSSGSSGSS | 14,333 | PERV_Q4VFZ2_3mut |
| GSSPAPGGS | 14,334 | MLVFF_P26809_3mut |
| PAPAPAP | 14,335 | AVIRE_P03360_3mutA |
| EAAAKEAAAKEAAAKEAAAK | 14,336 | WMSV_P03359_3mutA |
| PAPAPAPAP | 14,337 | SFV3L_P27401_2mutA |
| GGGGSS | 14,338 | MLVAV_P03356_3mutA |
| GSSGSSGSSGSSGSS | 14,339 | SFV3L_P27401_2mutA |
| PAPGGS | 14,340 | WMSV_P03359_3mutA |
| GSSEAAAKGGG | 14,341 | PERV_Q4VFZ2 |
| GSSGGSPAP | 14,342 | MLVMS_P03355_PLV919 |
| GSSGSSGSSGSSGSSGSS | 14,343 | SFV3L_P27401_2mutA |
| GGSGSSGGG | 14,344 | MLVCB_P08361_3mut |
| GGGPAPGSS | 14,345 | SFV3L_P27401-Pro_2mutA |
| GSSEAAAKGGS | 14,346 | WMSV_P03359_3mut |
| GSSEAAAKGGG | 14,347 | MLVAV_P03356_3mut |
| GGSGGGPAP | 14,348 | FFV_093209-Pro |
| GSSGSS | 14,349 | PERV_Q4VFZ2_3mut |
| PAPGGGGGS | 14,350 | GALV_P21414_3mutA |
| EAAAKPAPGGS | 14,351 | MLVAV_P03356_3mut |
| GSSGSS | 14,352 | MLVMS_P03355_3mut |
| EAAAKPAPGGS | 14,353 | FFV_O93209-Pro |
| GGGPAPEAAAK | 14,354 | MLVMS_P03355_3mutA_WS |
| GSSEAAAKGGG | 14,355 | MLVBM_Q7SVK7_3mut |
| GGGEAAAKGGS | 14,356 | BAEVM_P10272_3mut |
| GSSGSS | 14,357 | KORV_Q9TTC1-Pro_3mutA |
| EAAAKEAAAKEAAAK | 14,358 | SFV1_P23074 |
| PAPGSSGGS | 14,359 | KORV_Q9TTC1-Pro_3mut |
| PAPAPAPAPAP | 14,360 | MLVMS_P03355 |
| GSSEAAAK | 14,361 | SFV3L_P27401_2mut |
| PAP | | PERV_Q4VFZ2_3mut |
| GGSEAAAKGGG | 14,363 | MLVBM_Q7SVK7_3mut |
| GGSGGGPAP | 14,364 | MLVBM_Q7SVK7_3mutA_WS |
| GSSGSS | 14,365 | MLVMS_P03355_3mut |
| GGSEAAAK | 14,366 | MLVMS_P03355 |
| GSSEAAAKGGS | 14,367 | MLVMS_P03355_PLV919 |
| PAPGGGGGS | 14,368 | MLVFF_P26809_3mut |
| GSSGGG | 14,369 | PERV_Q4VFZ2_3mut |
| GSSGGS | 14,370 | PERV_Q4VFZ2_3mutA_WS |
| PAPGGG | 14,371 | BAEVM_P10272_3mut |
| PAPGSSGGG | 14,372 | MLVBM_Q7SVK7_3mut |
| GGSEAAAK | 14,373 | SFV3L_P27401_2mut |
| GSSPAPEAAAK | 14,374 | SFV3L_P27401-Pro_2mut |
| GSSGGSPAP | 14,375 | BAEVM_P10272_3mut |
| GGSPAPGSS | 14,376 | PERV_Q4VFZ2_3mutA_WS |
| GGSGGSGGS | 14,377 | PERV_Q4VFZ2 |
| GGSGGGPAP | 14,378 | FLV_P10273_3mut |
| GGGPAPEAAAK | 14,379 | SFV3L_P27401_2mutA |
| GGGGS | 14,380 | FLV_P10273_3mutA |
| GSSGGSGGG | 14,381 | XMRV6_A1Z651_3mut |
| EAAAKGGGGSS | 14,382 | PERV_Q4VFZ2 |
| GGSGSSGGG | 14,383 | SFV3L_P27401-Pro_2mutA |
| GGSGGSGGS | 14,384 | MLVFF_P26809_3mut |
| GGGPAPEAAAK | 14,385 | FLV_P10273_3mut |
| GSSGGGEAAAK | 14,386 | MLVMS_P03355_3mut |
| GGG | | SFV3L_P27401_2mut |
| GSAGSAAGSGEF | 14,388 | WMSV_P03359_3mut |
| GSSGGGPAP | 14,389 | MLVMS_P03355_PLV919 |
| GGGGSS | 14,390 | KORV_Q9TTC1-Pro_3mut |
| GGGGSSEAAAK | 14,391 | KORV_Q9TTC1 |
| PAPGGSGGG | 14,392 | SFV3L_P27401_2mut |
| GSSGSSGSSGSSGSS | 14,393 | FFV_093209 |
| GSSGGSPAP | 14,394 | MLVMS_P03355_3mut |
| GGSEAAAK | 14,395 | KORV_Q9TTC1-Pro_3mutA |
| GGGGSGGGGS | 14,396 | BAEVM_P10272_3mut |
| GSSEAAAKGGG | 14,397 | AVIRE_P03360_3mut |
| EAAAKPAPGGG | 14,398 | FLV_P10273_3mut |
| EAAAKGGSPAP | 14,399 | SFV3L_P27401-Pro_2mutA |
| GSSEAAAKPAP | 14,400 | MLVBM_Q7SVK7_3mut |
| GGGPAPGGS | 14,401 | MLVCB_P08361_3mut |
| GGG | | SFV3L_P27401_2mutA |
| EAAAKGGGGSEAAAK | 14,403 | SFV3L_P27401_2mutA |
| GGSGSSGGG | 14,404 | MLVBM_Q7SVK7_3mut |
| GSAGSAAGSGEF | 14,405 | BAEVM_P10272_3mut |
| GGGEAAAK | 14,406 | FOAMV_P14350_2mutA |
| PAPEAAAKGGS | 14,407 | WMSV_P03359_3mut |
| PAPAPAPAPAPAP | 14,408 | MLVF5_P26810_3mutA |
| GGSGGGGSS | 14,409 | FLV_P10273_3mutA |
| PAPGSSGGS | 14,410 | BAEVM_P10272_3mut |
| PAPEAAAK | 14,411 | WMSV_P03359_3mutA |
| GSSGSSGSSGSSGSSGSS | 14,412 | FFV_O93209-Pro_2mut |
| GGGGGSGSS | 14,413 | FFV_093209-Pro |
| GGGGGGGG | 14,414 | SFV3L_P27401-Pro_2mutA |
| GGGGGG | 14,415 | FLV_P10273_3mut |
| GSSGGSGGG | 14,416 | MLVAV_P03356_3mutA |
| GGGGSS | 14,417 | SFV3L_P27401-Pro_2mutA |
| GGSGGGPAP | 14,418 | FOAMV_P14350_2mut |
| GSSGSS | 14,419 | AVIRE_P03360_3mutA |
| EAAAKEAAAKEAAAKEAAAKEAAAK | 14,420 | SFV3L_P27401-Pro_2mutA |
| EAAAKEAAAK | 14,421 | BAEVM_P10272_3mut |
| GSSPAPEAAAK | 14,422 | GALV_P21414_3mutA |
| GGSEAAAKPAP | 14,423 | SFV3L_P27401_2mutA |
| GGSGGGEAAAK | 14,424 | SFV3L_P27401-Pro_2mutA |
| EAAAKGSSPAP | 14,425 | FOAMV_P14350_2mut |
| GGSGSSEAAAK | 14,426 | SFV3L_P27401_2mut |
| GGG | | PERV_Q4VFZ2 |
| GGGGGSGSS | 14,428 | FOAMV_P14350_2mut |
| GGSGGGEAAAK | 14,429 | KORV_Q9TTC1-Pro_3mut |
| GSSGGSGGG | 14,430 | AVIRE_P03360_3mutA |
| EAAAKPAPGGG | 14,431 | SFV3L_P27401_2mutA |
| PAPGGSGGG | 14,432 | KORV_Q9TTC1-Pro_3mut |
| PAPAPAP | 14,433 | WMSV_P03359_3mutA |
| GSSEAAAKPAP | 14,434 | SFV1_P23074 |
| SGGSSGGSSGSETPGTSESATPESSGGSSGGSS | 14,435 | SRV2_P51517 |
| GSSGGSGGG | 14,436 | PERV_Q4VFZ2_3mutA_WS |
| GSSGSSGSSGSSGSSGSS | 14,437 | FFV_093209 |
| GSSGGGPAP | 14,438 | WMSV_P03359_3mut |
| PAPAPAPAPAPAP | 14,439 | MLVBM_Q7SVK7_3mut |
| GGGGGSPAP | 14,440 | KORV_Q9TTC1-Pro_3mutA |
| PAPGSS | 14,441 | MLVBM_Q7SVK7_3mutA_WS |
| PAPEAAAKGGS | 14,442 | SFV3L_P27401-Pro_2mut |
| GGGGSSPAP | 14,443 | MLVMS_P03355_3mut |
| GGSEAAAK | 14,444 | FFV_O93209-Pro |
| EAAAKPAPGGS | 14,445 | AVIRE_P03360_3mutA |
| PAPGSS | 14,446 | WMSV_P03359_3mut |
| PAPGSSGGG | 14,447 | SFV3L_P27401-Pro_2mutA |
| EAAAKEAAAKEAAAK | 14,448 | SFV3L_P27401_2mut |
| GGS | | MLVRD_P11227_3mut |
| GGGGS | 14,450 | KORV_Q9TTC1-Pro_3mut |
| GGSGGGGSS | 14,451 | KORV_Q9TTC1 |
| GGSGGG | 14,452 | MLVMS_P03355_3mutA_WS |
| GGGEAAAKPAP | 14,453 | BAEVM_P10272_3mut |
| EAAAKEAAAKEAAAKEAAAKEAAAK | 14,454 | FLV_P10273 |
| PAPGGSGGG | 14,455 | KORV_Q9TTC1-Pro_3mutA |
| GSSGSSGSSGSSGSSGSS | 14,456 | HTL1L_POC211 |
| GGGEAAAKPAP | 14,457 | WMSV_P03359 |
| GSSGGSPAP | 14,458 | FFV_O93209-Pro |
| PAPAPAPAPAP | 14,459 | SFV3L_P27401-Pro_2mutA |
| GSSGGSEAAAK | 14,460 | SFV3L_P27401_2mutA |
| GGSPAPGSS | 14,461 | SFV3L_P27401_2mut |
| GGSGGSGGS | 14,462 | KORV_Q9TTC1-Pro_3mut |
| PAPEAAAKGSS | 14,463 | KORV_Q9TTC1-Pro_3mut |
| EAAAKGGS | 14,464 | KORV_Q9TTC1_3mutA |
| EAAAKGGGGSEAAAK | 14,465 | SFV3L_P27401-Pro_2mut |
| GGGGSSPAP | 14,466 | FFV_O93209-Pro |
| EAAAK | 14,467 | SFV3L_P27401_2mut |
| EAAAKGGGGSS | 14,468 | BAEVM_P10272_3mut |
| GGGGGSEAAAK | 14,469 | MLVBM_Q7SVK7_3mut |
| GGGG | 14,470 | PERV_Q4VFZ2 |
| GGGGGSEAAAK | 14,471 | FLV_P10273_3mut |
| EAAAKGGGPAP | 14,472 | KORV_Q9TTC1-Pro |
| GGGGGGGGSGGGGSGGGGS | 14,473 | FFV_O93209_2mutA |
| GSSGGSGGG | 14,474 | PERV_Q4VFZ2_3mut |
| GGGGSGGGGSGGGGS | 14,475 | GALV_P21414_3mutA |
| GGSGGGEAAAK | 14,476 | AVIRE_P03360_3mutA |
| PAPEAAAKGGG | 14,477 | SFV3L_P27401_2mut |
| GGGGSGGGGS | 14,478 | AVIRE_P03360 |
| GSSGGGEAAAK | 14,479 | SFV3L_P27401_2mutA |
| GGGGG | 14,480 | AVIRE_P03360_3mutA |
| GGSGSS | 14,481 | KORV_Q9TTC1_3mut |
| PAPAPAPAPAPAP | 14,482 | FOAMV_P14350_2mut |
| GGSEAAAKPAP | 14,483 | KORV_Q9TTC1-Pro_3mut |
| GGGGGG | 14,484 | PERV_Q4VFZ2_3mut |
| GSSGGGEAAAK | 14,485 | MLVBM_Q7SVK7 |
| SGGSSGGSSGSETPGTSESATPESSGGSSGGSS | 14,486 | MLVAV_P03356 |
| GGSPAPGSS | 14,487 | BAEVM_P10272_3mut |
| GGGGSSPAP | 14,488 | BAEVM_P10272 |
| GGGGSEAAAKGGGGS | 14,489 | SFV3L_P27401_2mut |
| GGGGGGGG | 14,490 | GALV_P21414_3mutA |
| PAPAP | 14,491 | MLVAV_P03356_3mut |
| GGGEAAAK | 14,492 | PERV_Q4VFZ2_3mutA_WS |
| GSSPAPGGG | 14,493 | FFV_O93209_2mut |
| GGSGGSGGSGGSGGS | 14,494 | BAEVM_P10272 |
| GGGGGS | 14,495 | MLVF5_P26810_3mutA |
| PAPGGGGSS | 14,496 | FLV_P10273_3mutA |
| GGGEAAAK | 14,497 | MLVBM_Q7SVK7_3mut |
| PAPEAAAKGGG | 14,498 | WMSV_P03359_3mut |
| GSSEAAAK | 14,499 | MLVBM_Q7SVK7_3mut |
| EAAAKEAAAK | 14,500 | AVIRE_P03360 |
| EAAAKGGGGGS | 14,501 | MLVBM_Q7SVK7_3mut |
| GGGEAAAKGGS | 14,502 | SFV3L_P27401-Pro_2mutA |
| PAPAPAPAPAP | 14,503 | MLVF5_P26810_3mut |
| PAPGSSEAAAK | 14,504 | SFV3L_P27401-Pro_2mutA |
| EAAAKEAAAKEAAAK | 14,505 | BAEVM_P10272_3mutA |
| GGSPAPGSS | 14,506 | MLVMS_P03355 |
| PAPGSSGGS | 14,507 | FLV_P10273_3mutA |
| EAAAKEAAAKEAAAKEAAAK | 14,508 | FOAMV_P14350-Pro_2mut |
| EAAAKGGG | 14,509 | KORV_Q9TTC1_3mutA |
| EAAAKGGSGGG | 14,510 | MLVBM_Q7SVK7_3mut |
| GGGGGS | 14,511 | KORV_Q9TTC1-Pro_3mutA |
| PAPGGSGGG | 14,512 | WMSV_P03359_3mut |
| GGGPAPGGS | 14,513 | KORV_Q9TTC1_3mutA |
| GSS | | FFV_093209 |
| GGSGGSGGS | 14,515 | PERV_Q4VFZ2_3mut |
| GGGGS | 14,516 | GALV_P21414_3mutA |
| GGGG | 14,517 | MLVF5_P26810_3mut |
| GGSEAAAKPAP | 14,518 | FFV_O93209-Pro_2mut |
| PAPAPAPAP | 14,519 | FFV_093209-Pro |
| PAP | | MLVF5_P26810_3mut |
| EAAAKEAAAKEAAAK | 14,521 | FFV_O93209_2mut |
| EAAAKGSS | 14,522 | MLVCB_P08361_3mut |
| EAAAKGGG | 14,523 | MLVBM_Q7SVK7_3mut |
| PAPEAAAKGGG | 14,524 | FFV_093209_2mut |
| GSSGGGEAAAK | 14,525 | SFV1_P23074-Pro_2mut |
| PAPGGGEAAAK | 14,526 | GALV_P21414_3mutA |
| GGGGSGGGGSGGGGSGGGGS | 14,527 | FOAMV_P14350-Pro_2mutA |
| GSSGGG | 14,528 | FOAMV_P14350_2mut |
| GGGGSGGGGSGGGGSGGGGS | 14,529 | SFV3L_P27401_2mutA |
| GGSGSS | 14,530 | AVIRE_P03360_3mut |
| GGSGSSEAAAK | 14,531 | MMTVB_P03365_WS |
| PAPAPAP | 14,532 | MLVAV_P03356_3mutA |
| GSSGGSPAP | 14,533 | SFV3L_P27401-Pro_2mut |
| GGSPAP | 14,534 | AVIRE_P03360 |
| GGSGGGPAP | 14,535 | FFV_O93209 |
| GSSEAAAK | 14,536 | PERV_Q4VFZ2 |
| GSSGGGPAP | 14,537 | PERV_Q4VFZ2_3mutA_WS |
| GGGGSSEAAAK | 14,538 | KORV_Q9TTC1_3mutA |
| GGSEAAAKPAP | 14,539 | SFVCP_Q87040 |
| GGSGGGPAP | 14,540 | FOAMV_P14350_2mutA |
| GGGGSGGGGSGGGGSGGGGS | 14,541 | BLVJ_P03361_2mutB |
| GGGGSSPAP | 14,542 | SFV3L_P27401_2mutA |
| EAAAKGGS | 14,543 | MLVF5_P26810_3mut |
| GGSEAAAKGSS | 14,544 | MLVCB_P08361_3mut |
| GGGGSSEAAAK | 14,545 | SFV3L_P27401_2mut |
| EAAAKGGSGGG | 14,546 | FOAMV_P14350_2mut |
| GGSGGS | 14,547 | FLV_P10273_3mut |
| EAAAKGGG | 14,548 | FFV_O93209-Pro |
| GSSGSSGSSGSSGSS | 14,549 | SFV3L_P27401 |
| GSSGGGPAP | 14,550 | PERV_Q4VFZ2_3mutA_WS |
| PAPGGSEAAAK | 14,551 | SFV3L_P27401-Pro_2mutA |
| GGSPAP | 14,552 | KORV_Q9TTC1 |
| EAAAKPAPGSS | 14,553 | KORV_Q9TTC1_3mutA |
| SGSETPGTSESATPES | 14,554 | SFV1_P23074 |
| GSSPAP | 14,555 | SFV3L_P27401-Pro_2mutA |
| GSSPAPGGG | 14,556 | SFV3L_P27401_2mut |
| GGGEAAAKGSS | 14,557 | SFV1_P23074_2mut |
| GGGPAPGGS | 14,558 | BAEVM_P10272_3mut |
| EAAAKGGG | 14,559 | KORV_Q9TTC1-Pro_3mutA |
| GSSGGG | 14,560 | SFV3L_P27401-Pro_2mut |
| GGSPAPEAAAK | 14,561 | BAEVM_P10272_3mut |
| EAAAKGSSPAP | 14,562 | FFV_093209 |
| EAAAKGGGGSEAAAK | 14,563 | SFV3L_P27401-Pro_2mutA |
| GSSGSSGSSGSSGSS | 14,564 | SFV1_P23074_2mut |
| EAAAKGGSPAP | 14,565 | FOAMV_P14350_2mut |
| GGSGGS | 14,566 | KORV_Q9TTC1-Pro_3mutA |
| EAAAKGSSGGS | 14,567 | GALV_P21414 |
| GSSGGGPAP | 14,568 | MLVAV_P03356 |
| PAPEAAAKGGS | 14,569 | FOAMV_P14350_2mut |
| EAAAKPAPGGG | 14,570 | AVIRE_P03360_3mut |
| GGSPAP | 14,571 | SFV3L_P27401_2mutA |
| GGGGSGGGGS | 14,572 | SFV3L_P27401_2mutA |
| GGGGSS | 14,573 | AVIRE_P03360_3mutA |
| GGSPAPGGG | 14,574 | SFV3L_P27401-Pro_2mutA |
| EAAAKPAPGSS | 14,575 | SFV3L_P27401 |
| EAAAKPAP | 14,576 | FOAMV_P14350-Pro_2mut |
| PAPEAAAKGSS | 14,577 | PERV_Q4VFZ2_3mutA_WS |
| EAAAKGGSGSS | 14,578 | SFV3L_P27401_2mutA |
| GGGEAAAKGSS | 14,579 | GALV_P21414_3mutA |
| GGGGSEAAAKGGGGS | 14,580 | PERV_Q4VFZ2_3mut |
| PAPGGSGSS | 14,581 | FFV_O93209-Pro_2mutA |
| GGSEAAAKPAP | 14,582 | GALV_P21414_3mutA |
| GGSGGSGGSGGSGGS | 14,583 | FFV_O93209-Pro |
| GSSGGSEAAAK | 14,584 | SFV3L_P27401-Pro_2mut |
| GGS | | GALV_P21414_3mutA |
| PAPGGSEAAAK | 14,586 | MLVMS_P03355 |
| PAPEAAAKGGS | 14,587 | BAEVM_P10272_3mutA |
| GGSGSSPAP | 14,588 | SFV3L_P27401-Pro_2mutA |
| GSSPAP | 14,589 | WMSV_P03359_3mut |
| GGGEAAAK | 14,590 | MMTVB_P03365 |
| GGGGSS | 14,591 | PERV_Q4VFZ2_3mut |
| GGSPAPGSS | 14,592 | SFV3L_P27401-Pro_2mut |
| PAPGGS | 14,593 | MLVBM_Q7SVK7_3mut |
| EAAAKGSSPAP | 14,594 | MLVBM_Q7SVK7_3mut |
| GGGGSSGGS | 14,595 | PERV_Q4VFZ2_3mut |
| PAPAPAPAPAPAP | 14,596 | SFV1_P23074 |
| GGSEAAAKGGG | 14,597 | SFV3L_P27401-Pro_2mut |
| GGSGGS | 14,598 | SFV1_P23074_2mut |
| GSSGGGGGS | 14,599 | MLVF5_P26810_3mutA |
| EAAAKGGGPAP | 14,600 | SFV3L_P27401 |
| EAAAKEAAAKEAAAKEAAAK | 14,601 | FOAMV_P14350-Pro_2mutA |
| GGGPAPGSS | 14,602 | SFV3L_P27401_2mutA |
| GGGGSGGGGSGGGGSGGGGS | 14,603 | SFV3L_P27401_2mut |
| EAAAKEAAAKEAAAKEAAAK | 14,604 | MMTVB_P03365_WS |
| PAPGSSGGS | 14,605 | KORV_Q9TTC1-Pro_3mutA |
| PAPGSSEAAAK | 14,606 | FOAMV_P14350-Pro_2mut |
| GSSPAPEAAAK | 14,607 | BAEVM_P10272_3mut |
| EAAAKGGGGSEAAAK | 14,608 | FFV_093209-Pro |
| GGSPAP | 14,609 | PERV_Q4VFZ2 |
| GGSGSSEAAAK | 14,610 | XMRV6_A1Z651_3mut |
| GGSEAAAKGGG | 14,611 | GALV_P21414_3mutA |
| PAPGGGGSS | 14,612 | AVIRE_P03360_3mutA |
| GGSGGSGGSGGS | 14,613 | PERV_Q4VFZ2 |
| GGGGSSGGS | 14,614 | PERV_Q4VFZ2_3mutA_WS |
| SGGSSGGSSGSETPGTSESATPESSGGSSGGSS | 14,615 | BAEVM_P10272_3mutA |
| GGGPAP | 14,616 | MLVAV_P03356_3mut |
| GGGGSGGGGSGGGGSGGGGS | 14,617 | FFV_093209_2mut |
| GSSEAAAK | 14,618 | FFV_O93209 |
| GGSPAPEAAAK | 14,619 | FOAMV_P14350_2mut |
| GGGGGSEAAAK | 14,620 | FOAMV_P14350_2mut |
| GSSPAPGGS | 14,621 | MLVBM_Q7SVK7_3mut |
| GSS | | SFVCP_Q87040_2mut |
| EAAAKPAP | 14,623 | FOAMV_P14350-Pro |
| EAAAKGGG | 14,624 | SFV3L_P27401_2mut |
| GGGEAAAK | 14,625 | AVIRE_P03360_3mutA |
| PAPGSSGGG | 14,626 | WMSV_P03359_3mut |
| EAAAKGGSPAP | 14,627 | SFV3L_P27401 |
| GSSGGSGGG | 14,628 | SFV3L_P27401-Pro_2mutA |
| GSSGGGEAAAK | 14,629 | GALV_P21414_3mutA |
| GGGPAPGSS | 14,630 | MLVBM_Q7SVK7_3mutA_WS |
| PAPGGGEAAAK | 14,631 | FFV_O93209-Pro_2mut |
| GSSGSSGSSGSS | 14,632 | SFV1_P23074_2mut |
| GGSEAAAK | 14,633 | PERV_Q4VFZ2_3mutA_WS |
| GGGEAAAKPAP | 14,634 | SFV3L_P27401_2mut |
| EAAAKGGGPAP | 14,635 | SFV3L_P27401_2mut |
| GGGGSSPAP | 14,636 | FLV_P10273_3mut |
| EAAAKPAPGSS | 14,637 | FFV_093209_2mut |
| GGGGSSPAP | 14,638 | SFV3L_P27401_2mut |
| GSSGSS | 14,639 | KORV_Q9TTC1_3mutA |
| GGGGSGGGGSGGGGSGGGGSGGGGS | 14,640 | BLVJ_P03361_2mut |
| GGGGSSGGS | 14,641 | GALV_P21414_3mutA |
| EAAAKGGSGSS | 14,642 | FFV_O93209-Pro |
| EAAAKPAP | 14,643 | PERV_Q4VFZ2 |
| GSSGGGEAAAK | 14,644 | MLVBM_Q7SVK7_3mut |
| PAPGGSGGG | 14,645 | BAEVM_P10272 |
| EAAAKGGGPAP | 14,646 | MLVF5_P26810 |
| GSSGSSGSS | 14,647 | MLVBM_Q7SVK7_3mut |
| GSSGGS | 14,648 | AVIRE_P03360_3mutA |
| GGSEAAAKGGG | 14,649 | FOAMV_P14350_2mut |
| EAAAKGGS | 14,650 | MLVF5_P26810_3mutA |
| GGSGSSGGG | 14,651 | WMSV_P03359_3mut |
| EAAAK | 14,652 | SFV1_P23074_2mut |
| GSSGGSPAP | 14,653 | SFV3L_P27401-Pro_2mutA |
| GGGGSSGGS | 14,654 | KORV_Q9TTC1_3mut |
| PAPGGSGGG | 14,655 | FFV_O93209-Pro_2mut |
| GGGPAPGGS | 14,656 | SFV3L_P27401_2mutA |
| GSSPAPEAAAK | 14,657 | FLV_P10273_3mut |
| GGSGSSPAP | 14,658 | SFV3L_P27401_2mut |
| GSSEAAAKGGS | 14,659 | SFV3L_P27401_2mut |
| PAPGGG | 14,660 | SFV3L_P27401_2mutA |
| SGSETPGTSESATPES | 14,661 | KORV_Q9TTC1-Pro_3mut |
| GGGGS | 14,662 | SFV1_P23074-Pro_2mutA |
| GSSGGGEAAAK | 14,663 | WMSV_P03359 |
| EAAAKGGGGSEAAAK | 14,664 | MLVF5_P26810_3mutA |
| GSSEAAAKPAP | 14,665 | FFV_093209 |
| GGGGGG | 14,666 | SFV1_P23074_2mutA |
| EAAAKEAAAKEAAAK | 14,667 | MMTVB_P03365-Pro |
| EAAAKPAPGSS | 14,668 | MLVBM_Q7SVK7_3mut |
| GGSGSSEAAAK | 14,669 | SFV3L_P27401_2mutA |
| GGSEAAAK | 14,670 | MLVMS_P03355_3mut |
| GGSPAPEAAAK | 14,671 | SFV3L_P27401_2mut |
| GGGPAPGSS | 14,672 | SFV1_P23074 |
| GGGGGSEAAAK | 14,673 | MLVBM_Q7SVK7_3mutA_WS |
| EAAAKPAPGSS | 14,674 | KORV_Q9TTC1-Pro |
| GSSGSSGSSGSS | 14,675 | SFV3L_P27401_2mut |
| EAAAKPAP | 14,676 | SFV3L_P27401_2mut |
| GGGEAAAK | 14,677 | PERV_Q4VFZ2_3mut |
| GGSGGS | 14,678 | SFV3L_P27401_2mutA |
| EAAAKGSSGGS | 14,679 | MMTVB_P03365 |
| SGSETPGTSESATPES | 14,680 | SFV3L_P27401 |
| EAAAKGSSGGG | 14,681 | PERV_Q4VFZ2 |
| EAAAKEAAAKEAAAKEAAAKEAAAKEAAAK | 14,682 | MMTVB_P03365 |
| GGSGGGPAP | 14,683 | KORV_Q9TTC1_3mutA |
| PAPAPAPAP | 14,684 | SFV3L_P27401 |
| GGGEAAAKGGS | 14,685 | SFV1_P23074_2mut |
| GSSGGSGGG | 14,686 | PERV_Q4VFZ2_3mut |
| PAPEAAAKGGS | 14,687 | FOAMV_P14350_2mutA |
| GGGEAAAKGSS | 14,688 | SFV3L_P27401_2mut |
| GGGGSGGGGSGGGGSGGGGS | 14,689 | MLVBM_Q7SVK7 |
| PAPGSSGGG | 14,690 | FLV_P10273 |
| GGSGSSGGG | 14,691 | FFV_093209 |
| EAAAKPAPGSS | 14,692 | MLVBM_Q7SVK7 |
| GSSEAAAKGGG | 14,693 | SFV3L_P27401_2mutA |
| GGSGGSGGSGGSGGS | 14,694 | MLVF5_P26810 |
| GGSEAAAKPAP | 14,695 | SFV3L_P27401-Pro_2mutA |
| EAAAKGGSPAP | 14,696 | SFV3L_P27401_2mutA |
| EAAAKGGGGGS | 14,697 | SFV3L_P27401_2mut |
| GSSPAPEAAAK | 14,698 | SFV3L_P27401_2mutA |
| PAPAP | 14,699 | MLVBM_Q7SVK7_3mut |
| PAPGGSEAAAK | 14,700 | KORV_Q9TTC1-Pro |
| GGSGSS | 14,701 | MLVF5_P26810_3mutA |
| GGSEAAAKPAP | 14,702 | FFV_093209_2mut |
| GSS | | MLVMS_P03355 |
| SGGSSGGSSGSETPGTSESATPESSGGSSGGSS | 14,704 | SFV3L_P27401-Pro |
| PAPGGGEAAAK | 14,705 | SFV3L_P27401_2mut |
| PAPGGGGGS | 14,706 | SFV3L_P27401-Pro_2mut |
| PAPGGSGSS | 14,707 | BAEVM_P10272_3mut |
| GSSGGGEAAAK | 14,708 | FFV_O93209 |
| GGSEAAAKPAP | 14,709 | SFV1_P23074_2mut |
| GGGGG | 14,710 | FLV_P10273_3mut |
| GGGEAAAKGSS | 14,711 | SFV3L_P27401 |
| GSSGSSGSSGSSGSS | 14,712 | SFV1_P23074-Pro |
| SGSETPGTSESATPES | 14,713 | AVIRE_P03360 |
| PAPGSSGGG | 14,714 | MLVBM_Q7SVK7_3mut |
| GGGGSSPAP | 14,715 | HTL3P_Q4U0X6_2mut |
| GGGEAAAK | 14,716 | SFV1_P23074 |
| GGSGGG | 14,717 | AVIRE_P03360 |
| EAAAKGSSGGG | 14,718 | SFV3L_P27401_2mutA |
| GSSPAPEAAAK | 14,719 | FOAMV_P14350-Pro_2mutA |
| GGGPAPGSS | 14,720 | WMSV_P03359 |
| EAAAKGSSGGG | 14,721 | MLVMS_P03355 |
| GGGGGSEAAAK | 14,722 | MLVMS_P03355 |
| EAAAKPAPGGS | 14,723 | SFV3L_P27401 |
| EAAAKGSSPAP | 14,724 | SFV3L_P27401 |
| GGGGGGG | 14,725 | FOAMV_P14350_2mutA |
| EAAAKEAAAKEAAAK | 14,726 | SFV3L_P27401 |
| GSSPAPGGS | 14,727 | FFV_093209_2mutA |
| GGGGSSEAAAK | 14,728 | SFV3L_P27401-Pro_2mutA |
| GGSEAAAKGSS | 14,729 | GALV_P21414_3mutA |
| GGSEAAAKGSS | 14,730 | BAEVM_P10272_3mutA |
| EAAAKPAPGGG | 14,731 | MLVCB_P08361 |
| GSSGSSGSSGSSGSSGSS | 14,732 | SFV1_P23074-Pro |
| GGGGSEAAAKGGGGS | 14,733 | FOAMV_P14350_2mut |
| GSSPAPGGS | 14,734 | MLVMS_P03355_PLV919 |
| GGGGSGGGGS | 14,735 | FFV_O93209-Pro |
| GSSGGSPAP | 14,736 | KORV_Q9TTC1_3mutA |
| GGSGGS | 14,737 | GALV_P21414_3mutA |
| PAPGSSEAAAK | 14,738 | WMSV_P03359 |
| PAPGGGGSS | 14,739 | MMTVB_P03365-Pro |
| GGGGSSGGS | 14,740 | PERV_Q4VFZ2_3mutA_WS |
| GGGGSGGGGS | 14,741 | FFV_093209_2mut |
| GGGGSGGGGSGGGGSGGGGS | 14,742 | XMRV6_A1Z651 |
| GGSGSSEAAAK | 14,743 | SFV1_P23074_2mut |
| GGSGGGGSS | 14,744 | GALV_P21414_3mutA |
| GGSEAAAKPAP | 14,745 | MLVBM_Q7SVK7 |
| EAAAKGGSPAP | 14,746 | SFV1_P23074_2mutA |
| PAPAPAPAP | 14,747 | FFV_093209 |
| GSSGGSPAP | 14,748 | MMTVB_P03365-Pro |
| GGGGGSPAP | 14,749 | KORV_Q9TTC1_3mutA |
| EAAAKGGGPAP | 14,750 | PERV_Q4VFZ2 |
| GSSGGSPAP | 14,751 | BAEVM_P10272 |
| GGGGG | 14,752 | FFV_093209 |
| GGGGGS | 14,753 | FLV_P10273_3mutA |
| EAAAKEAAAKEAAAK | 14,754 | FOAMV_P14350 |
| PAPGGG | 14,755 | MLVCB_P08361_3mut |
| GSSGGSEAAAK | 14,756 | FOAMV_P14350_2mutA |
| GGSPAPGGG | 14,757 | FLV_P10273_3mut |
| GSSGSSGSSGSSGSSGSS | 14,758 | SFV1_P23074-Pro_2mutA |
| GGSPAPEAAAK | 14,759 | SFV3L_P27401 |
| PAPGGGGSS | 14,760 | HTL3P_Q4U0X6_2mutB |
| GGGGSSEAAAK | 14,761 | MMTVB_P03365_2mut_WS |
| PAPGGS | 14,762 | MLVRD_P11227_3mut |
| GGSGGSGGSGGSGGS | 14,763 | MMTVB_P03365 |
| GSAGSAAGSGEF | 14,764 | AVIRE_P03360 |
| GSSGGS | 14,765 | BAEVM_P10272_3mutA |
| GGSGGGGSS | 14,766 | MMTVB_P03365 |
| GGSGGGGSS | 14,767 | WMSV_P03359 |
| PAPEAAAKGSS | 14,768 | SFV1_P23074 |
| GSSGSSGSSGSS | 14,769 | SFV1_P23074-Pro_2mutA |
| PAPAPAPAPAPAP | 14,770 | SFV3L_P27401 |
| PAPGSSGGG | 14,771 | FLV_P10273_3mut |
| GGSGSSPAP | 14,772 | MLVMS_P03355 |
| GGSGGGPAP | 14,773 | FOAMV_P14350 |
| PAPGGGGGS | 14,774 | KORV_Q9TTC1_3mutA |
| EAAAKGSSPAP | 14,775 | GALV_P21414_3mutA |
| GGSGSSPAP | 14,776 | MLVBM_Q7SVK7_3mut |
| EAAAKGSS | 14,777 | SFV3L_P27401_2mut |
| GGGGGSEAAAK | 14,778 | WMSV_P03359 |
| GGGGGGGG | 14,779 | SFV1_P23074-Pro |
| EAAAKEAAAK | 14,780 | MLVBM_Q7SVK7 |
| GGGEAAAKGGS | 14,781 | MLVBM_Q7SVK7 |
| EAAAKGGSPAP | 14,782 | SFV3L_P27401_2mut |
| GSSEAAAK | 14,783 | XMRV6_A1Z651 |
| PAPGGGEAAAK | 14,784 | MMTVB_P03365_WS |
| GGSPAP | 14,785 | GALV_P21414_3mutA |
| GSSPAPGGG | 14,786 | MLVBM_Q7SVK7_3mutA_WS |
| GGSGSSPAP | 14,787 | SFV1_P23074_2mutA |
| GGS | | HTL32_Q0R5R2_2mut |
| GGSGGGGSS | 14,789 | MMTVB_P03365-Pro |
| GGGGSGGGGSGGGGSGGGGS | 14,790 | SFVCP_Q87040_2mutA |
| EAAAKGGGPAP | 14,791 | FOAMV_P14350_2mut |
| GSSGGGEAAAK | 14,792 | MMTVB_P03365 |
| SGGSSGGSSGSETPGTSESATPESSGGSSGGSS | 14,793 | MLVBM_Q7SVK7_3mutA_WS |
| AEAAAKEAAAKEAAAKEAAAKALEAEAAAKEAAAKEAAAKEAAAKA | 14,794 | MMTVB_P03365_WS |
| EAAAKEAAAK | 14,795 | FOAMV_P14350-Pro_2mut |
| GSSPAPEAAAK | 14,796 | FOAMV_P14350_2mutA |
| EAAAKPAPGGS | 14,797 | GALV_P21414_3mutA |
| GSSGGSPAP | 14,798 | KORV_Q9TTC1-Pro_3mut |
| GGGPAPEAAAK | 14,799 | MLVAV_P03356 |
| GGGEAAAKPAP | 14,800 | SFV1_P23074-Pro_2mut |
| GGGGGSEAAAK | 14,801 | SFV3L_P27401_2mut |
| GGGPAPGSS | 14,802 | SFV3L_P27401_2mut |
| GGSEAAAKPAP | 14,803 | AVIRE_P03360 |
| GSSGSSGSSGSSGSSGSS | 14,804 | SFV1_P23074-Pro_2mut |
| EAAAKGSSGGS | 14,805 | FOAMV_P14350_2mutA |
| GGGGGG | 14,806 | MLVBM_Q7SVK7_3mut |
| GSSPAPGGS | 14,807 | PERV_Q4VFZ2 |
| GGSGSSPAP | 14,808 | GALV_P21414_3mutA |
| GGGPAPEAAAK | 14,809 | SFV3L_P27401 |
| GGSGGGEAAAK | 14,810 | WMSV_P03359 |
| GSAGSAAGSGEF | 14,811 | SFV1_P23074_2mut |
| GSSGGGEAAAK | 14,812 | MLVMS_P03355 |
| GGG | | MMTVB_P03365-Pro |
| PAPGSSGGS | 14,814 | FOAMV_P14350_2mut |
| GGGGSSPAP | 14,815 | FFV_093209_2mut |
| SGGSSGGSSGSETPGTSESATPESSGGSSGGSS | 14,816 | MMTVB_P03365_WS |
| GGGGGGG | 14,817 | XMRV6_A1Z651 |
| PAPAPAPAPAP | 14,818 | FOAMV_P14350 |
| GGGGSGGGGSGGGGSGGGGS | 14,819 | MMTVB_P03365_2mut_WS |
| GGSGGGPAP | 14,820 | SFV3L_P27401_2mut |
| GGGGGG | 14,821 | SFV1_P23074-Pro |
| EAAAKPAPGSS | 14,822 | SFV3L_P27401_2mut |
| GGGGSSGGS | 14,823 | HTL3P_Q4U0X6_2mut |
| PAPGSSEAAAK | 14,824 | MMTVB_P03365-Pro |
| GGGGSSPAP | 14,825 | FOAMV_P14350-Pro_2mut |
| PAPGSSGGS | 14,826 | MMTVB_P03365 |
| AEAAAKEAAAKEAAAKEAAAKALEAEAAAKEAAAKEAAAKEAAAKA | 14,827 | SRV2_P51517 |
| PAPAPAP | 14,828 | MMTVB_P03365_2mut_WS |
| PAPGGGGGS | 14,829 | MMTVB_P03365_2mutB |
| GGGGSS | 14,830 | SFV1_P23074-Pro_2mutA |
| EAAAKEAAAKEAAAKEAAAK | 14,831 | SFV3L_P27401-Pro |
| GGSGGSGGSGGSGGS | 14,832 | MMTVB_P03365-Pro |
| GGGGGGG | 14,833 | SFV3L_P27401_2mut |
| PAPGGGEAAAK | 14,834 | SFV3L_P27401 |
| PAPGSS | 14,835 | FOAMV_P14350_2mutA |
| GGGGSGGGGS | 14,836 | SFVCP_Q87040_2mutA |
| GSSGGSGGG | 14,837 | XMRV6_A1Z651 |
| GGGGSGGGGSGGGGSGGGGSGGGGSGGGGS | 14,838 | MLVBM_Q7SVK7 |
| GSSEAAAKGGG | 14,839 | FFV_O93209-Pro_2mut |
| GGSEAAAKPAP | 14,840 | SFV3L_P27401-Pro |
| GSSGGSGGG | 14,841 | SFV1_P23074_2mut |
| EAAAKGGGGSS | 14,842 | FOAMV_P14350_2mutA |
| GGGGGG | 14,843 | SFV3L_P27401_2mut |
| GGGGG | 14,844 | MLVBM_Q7SVK7_3mut |
| PAPEAAAKGGG | 14,845 | SFV3L_P27401 |
| EAAAKGGSPAP | 14,846 | KORV_Q9TTC1_3mutA |
| GGGEAAAKPAP | 14,847 | SFV1_P23074_2mut |
| GSSGSSGSSGSSGSSGSS | 14,848 | KORV_Q9TTC1-Pro |
| EAAAKEAAAKEAAAKEAAAK | 14,849 | SFVCP_Q87040 |
| PAPGSSEAAAK | 14,850 | MLVBM_Q7SVK7 |
| GSSGSSGSS | 14,851 | FFV_O93209-Pro_2mut |
| GSSGGGPAP | 14,852 | SFV3L_P27401-Pro_2mut |
| GGGPAPEAAAK | 14,853 | WMSV_P03359_3mut |
| GGGEAAAK | 14,854 | MMTVB_P03365-Pro |
| GSSGSSGSSGSS | 14,855 | SFV3L_P27401-Pro_2mutA |
| PAPAPAPAPAP | 14,856 | FFV_O93209-Pro |
| GGSPAPEAAAK | 14,857 | FFV_O93209-Pro_2mut |
| GSSGSSGSSGSSGSSGSS | 14,858 | GALV_P21414 |
| EAAAKEAAAKEAAAKEAAAKEAAAK | 14,859 | FOAMV_P14350 |
| GGGPAPEAAAK | 14,860 | MMTVB_P03365-Pro |
| PAPGGSGGG | 14,861 | MLVF5_P26810_3mutA |
| PAPGGSGGG | 14,862 | FLV_P10273_3mut |
| GGGEAAAKGGS | 14,863 | SFV3L_P27401 |
| GSAGSAAGSGEF | 14,864 | MLVBM_Q7SVK7_3mut |
| GSSPAPGGG | 14,865 | MPMV_P07572_2mutB |
| GSSGSSGSSGSSGSSGSS | 14,866 | FOAMV_P14350 |
| GGSGGGGSS | 14,867 | BLVJ_P03361_2mut |
| PAPEAAAKGSS | 14,868 | SFV1_P23074-Pro |
| GGG | | FFV_O93209 |
| EAAAKGGGGSS | 14,870 | SFV1_P23074_2mut |
| EAAAKEAAAKEAAAKEAAAKEAAAKEAAAK | 14,871 | SRV2_P51517 |
| GGGGSGGGGSGGGGSGGGGSGGGGSGGGGS | 14,872 | MMTVB_P03365 |
| GGGEAAAKGGS | 14,873 | MMTVB_P03365_WS |
| GSSGSS | 14,874 | SFV1_P23074 |
| GSSGGGGGS | 14,875 | SFV3L_P27401 |
| GGGGSSEAAAK | 14,876 | SFV1_P23074 |
| EAAAKGSSGGS | 14,877 | HTL1A_P03362_2mutB |
| GSSEAAAKGGS | 14,878 | GALV_P21414_3mutA |
| EAAAKGSSPAP | 14,879 | SFV1_P23074 |
| EAAAKPAPGSS | 14,880 | SFV3L_P27401_2mutA |
| PAPGSSGGG | 14,881 | SFV3L_P27401-Pro_2mut |
| GGGGSGGGGSGGGGSGGGGSGGGGSGGGGS | 14,882 | SFV3L_P27401-Pro |
| EAAAKEAAAKEAAAKEAAAKEAAAK | 14,883 | MMTVB_P03365_WS |
| GGGGSSEAAAK | 14,884 | MLVF5_P26810_3mutA |
| EAAAKGGSPAP | 14,885 | GALV_P21414 |
| PAPEAAAKGSS | 14,886 | MMTVB_P03365_WS |
| GSSGGGGGS | 14,887 | SFVCP_Q87040_2mut |
| GGGGSSPAP | 14,888 | SFV1_P23074 |
| EAAAKGGGGSS | 14,889 | XMRV6_A1Z651 |
| PAPAPAPAP | 14,890 | MMTVB_P03365 |
| GGSEAAAKGSS | 14,891 | SFV3L_P27401_2mutA |
| GSSPAPGGG | 14,892 | MMTVB_P03365_WS |
| GGGGGG | 14,893 | SFV3L_P27401-Pro |
| GGSGGSGGS | 14,894 | FOAMV_P14350-Pro_2mut |
| PAPAPAPAPAPAP | 14,895 | WMSV_P03359 |
| GSSPAP | 14,896 | MLVBM_Q7SVK7 |
| GGGGGSGSS | 14,897 | MMTVB_P03365_2mut_WS |
| EAAAKGSSGGS | 14,898 | MMTVB_P03365_2mutB_WS |
| EAAAK | 14,899 | FFV_O93209_2mutA |
| PAPEAAAK | 14,900 | SFV1_P23074-Pro |
| EAAAKGGSGSS | 14,901 | SFV3L_P27401 |
| GGSGGSGGS | 14,902 | FFV_O93209-Pro |
| GSSGGGEAAAK | 14,903 | MMTVB_P03365 |
| SGGSSGGSSGSETPGTSESATPESSGGSSGGSS | 14,904 | MLVFF_P26809_3mutA |
| GGSGGSGGSGGSGGSGGS | 14,905 | HTL1L_POC211_2mutB |
| GGGEAAAK | 14,906 | SFV3L_P27401-Pro_2mutA |
| GGGGGSGSS | 14,907 | MMTVB_P03365 |
| GSSPAPGGS | 14,908 | FOAMV_P14350_2mutA |
| EAAAKGSS | 14,909 | MLVMS_P03355 |
| GSSGGSGGG | 14,910 | FFV_O93209-Pro |
| GGSGGGGSS | 14,911 | MMTVB_P03365-Pro_2mut |
| GGSPAPGSS | 14,912 | FOAMV_P14350_2mut |
| GGSGGSGGSGGSGGSGGS | 14,913 | SFVCP_Q87040-Pro_2mut |
| GSSEAAAKGGG | 14,914 | FOAMV_P14350_2mutA |
| GGSGGSGGS | 14,915 | MMTVB_P03365-Pro |
| GSSGSSGSSGSSGSSGSS | 14,916 | MMTVB_P03365_2mut_WS |
| GSSGSSGSSGSSGSS | 14,917 | MMTVB_P03365-Pro |
| PAPEAAAK | 14,918 | WDSV_O92815 |
| GSSGSSGSSGSSGSS | 14,919 | FFV_O93209-Pro_2mut |
| EAAAKGGGGSEAAAK | 14,920 | MMTVB_P03365-Pro |
| GGSPAPEAAAK | 14,921 | FOAMV_P14350 |
| GSSGSS | 14,922 | PERV_Q4VFZ2 |
| GGG | | MMTVB_P03365-Pro |
| GGGGSGGGGSGGGGS | 14,924 | FFV_O93209_2mut |
| EAAAKEAAAKEAAAKEAAAKEAAAKEAAAK | 14,925 | MMTVB_P03365-Pro |
| GGSGSSPAP | 14,926 | WMSV_P03359 |
| GGGGGGGG | 14,927 | SFV3L_P27401_2mut |
| PAPGSSEAAAK | 14,928 | FOAMV_P14350-Pro_2mutA |
| GGGGSSPAP | 14,929 | FOAMV_P14350_2mut |
| GSSGGSPAP | 14,930 | MLVBM_Q7SVK7_3mut |
| GSSGGGGGS | 14,931 | GALV_P21414_3mutA |
| EAAAKEAAAKEAAAKEAAAKEAAAK | 14,932 | MMTVB_P03365 |
| GSSGGGGGS | 14,933 | SFV1_P23074_2mut |
| GGGGSEAAAKGGGGS | 14,934 | SFV1_P23074 |
| GGGEAAAKPAP | 14,935 | FFV_093209 |
| PAPGGGEAAAK | 14,936 | SFV1_P23074 |
| GGSGGGEAAAK | 14,937 | PERV_Q4VFZ2_3mutA_WS |
| GSSGGG | 14,938 | MMTVB_P03365-Pro |
| EAAAKGSSGGS | 14,939 | FFV_093209_2mut |
| GGGGG | 14,940 | SFV1_P23074_2mut |
| GGGPAP | 14,941 | SFV3L_P27401 |
| GSSGGSEAAAK | 14,942 | FFV_093209 |
| SGGSSGGSSGSETPGTSESATPESSGGSSGGSS | 14,943 | MMTVB_P03365-Pro |
| GSSGGGEAAAK | 14,944 | SFV1_P23074_2mutA |
| GSSGSSGSSGSSGSS | 14,945 | SFV3L_P27401_2mut |
| GGSEAAAKPAP | 14,946 | FLV_P10273 |
| GGGGSGGGGS | 14,947 | FOAMV_P14350-Pro_2mutA |
| GSSEAAAKPAP | 14,948 | SFV3L_P27401 |
| GGGGSEAAAKGGGGS | 14,949 | MMTVB_P03365-Pro |
| PAPGSSEAAAK | 14,950 | MLVF5_P26810_3mut |
| EAAAKGGSGGG | 14,951 | SFV3L_P27401 |
| GGGPAPGGS | 14,952 | SFV3L_P27401 |
| GSSEAAAKGGS | 14,953 | FOAMV_P14350_2mutA |
| EAAAKGGSGGG | 14,954 | HTL1L_POC211 |
| GSSGGSPAP | 14,955 | SFV3L_P27401_2mutA |
| PAPAP | 14,956 | FFV_O93209 |
| PAPGGSGSS | 14,957 | MMTVB_P03365_WS |
| EAAAKGGGGGS | 14,958 | FOAMV_P14350_2mut |
| PAPEAAAKGGS | 14,959 | SFV3L_P27401_2mut |
| GSSEAAAKPAP | 14,960 | MMTVB_P03365-Pro |
| GGSGGS | 14,961 | PERV_Q4VFZ2_3mut |
| GSSEAAAKGGG | 14,962 | FFV_O93209-Pro_2mutA |
| EAAAK | 14,963 | HTL1L_POC211 |
| GSSPAP | 14,964 | MLVMS_P03355 |
| EAAAKPAPGGG | 14,965 | FFV_093209-Pro_2mut |
| GGGGSEAAAKGGGGS | 14,966 | SFV1_P23074-Pro_2mut |
| EAAAKGSSGGS | 14,967 | SFV3L_P27401 |
| GSAGSAAGSGEF | 14,968 | FFV_O93209_2mutA |
| PAPEAAAKGGS | 14,969 | MMTVB_P03365_2mutB_WS |
| EAAAKEAAAKEAAAKEAAAKEAAAKEAAAK | 14,970 | MMTVB_P03365 |
| GGS | | MMTVB_P03365 |
| GGSEAAAKPAP | 14,972 | SFV1_P23074 |
| EAAAKGSSGGG | 14,973 | HTLV2_P03363_2mut |
| GGSEAAAKGGG | 14,974 | MMTVB_P03365_WS |
| GGSGGS | 14,975 | FFV_O93209-Pro |
| GSSEAAAKGGS | 14,976 | MMTVB_P03365-Pro |
| PAPAPAPAPAP | 14,977 | SFV1_P23074_2mutA |
| GGSEAAAKGGG | 14,978 | MMTVB_P03365_2mutB_WS |
| PAPAPAPAP | 14,979 | MMTVB_P03365_WS |
| GGGGSGGGGSGGGGSGGGGSGGGGS | 14,980 | HTL3P_Q4U0X6_2mut |
| PAPGGSEAAAK | 14,981 | SFV1_P23074-Pro_2mut |
| GGSGGGPAP | 14,982 | MMTVB_P03365 |
| GSSGSSGSSGSSGSSGSS | 14,983 | MMTVB_P03365-Pro |
| GGSEAAAKPAP | 14,984 | SFV1_P23074-Pro |
| GGGEAAAKGSS | 14,985 | SFV3L_P27401_2mutA |
| GGGPAPGGS | 14,986 | AVIRE_P03360 |
| PAPGGG | 14,987 | MLVRD_P11227 |
| GGSEAAAKGSS | 14,988 | SFV3L_P27401_2mut |
| GGGEAAAKGSS | 14,989 | FOAMV_P14350_2mut |
| GGGEAAAKGSS | 14,990 | SFV1_P23074-Pro |
| EAAAKEAAAKEAAAKEAAAK | 14,991 | MLVAV_P03356 |
| EAAAKGGGPAP | 14,992 | JSRV_P31623_2mutB |
| EAAAKGGGGSS | 14,993 | FOAMV_P14350_2mut |
| EAAAKEAAAKEAAAKEAAAKEAAAK | 14,994 | SRV2_P51517 |
| GSSGGGGGS | 14,995 | FFV_O93209 |
| PAPAPAP | 14,996 | FOAMV_P14350_2mutA |
| GGSGGSGGSGGS | 14,997 | FOAMV_P14350 |
| GGGEAAAK | 14,998 | MMTVB_P03365_WS |
| GGGGGS | 14,999 | SFV1_P23074_2mutA |
| GGSGGS | 15,000 | WMSV_P03359_3mut |
| EAAAKGGS | 15,001 | MMTVB_P03365-Pro |
| GGGGSS | 37645 | BLVJ_P03361_2mut |
| PAPAP | 37646 | MMTVB_P03365-Pro_2mut |
| PAPGGG | 37647 | SMRVH_P03364 |
| EAAAKGGGGSS | 37648 | SFV3L_P27401 |
| PAPAPAPAPAP | 37649 | MMTVB_P03365 |
| GGGPAP | 37650 | MMTVB_P03365-Pro |
| GSSGGSGGG | 37651 | MMTVB_P03365 |
| EAAAKGGGPAP | 37652 | FOAMV_P14350_2mutA |
| GSSGSSGSSGSS | 37654 | SFV1_P23074 |
| GGGGSGGGGS | 37655 | SFV3L_P27401 |
| GSSGGSGGG | 15,012 | MLVF5_P26810 |
| GGGEAAAKPAP | 15,013 | MMTVB_P03365-Pro |
| PAPEAAAK | 15,014 | HTLV2_P03363_2mut |
| GSSGSSGSSGSS | 15,015 | FOAMV_P14350_2mut |
| GSSEAAAKPAP | 15,016 | MMTVB_P03365-Pro |
| PAPEAAAKGGG | 15,017 | HTL3P_Q4U0X6_2mut |
| GGSEAAAKGSS | 15,018 | MMTVB_P03365-Pro |
| EAAAKPAPGGS | 15,019 | MMTVB_P03365_2mut_WS |
| GSSGGSEAAAK | 15,020 | MLVF5_P26810_3mutA |
| GGGGSGGGGSGGGGSGGGGSGGGGSGGGGS | 15,021 | MLVF5_P26810_3mut |
| EAAAKGGGGSS | 15,022 | MMTVB_P03365-Pro |
| GGGGGSGSS | 15,023 | HTL1A_P03362_2mutB |
| PAPAP | 15,024 | FFV_O93209-Pro_2mut |
| GGGGGSPAP | 15,025 | HTL1C_P14078_2mut |
| GGGPAP | 15,026 | HTLV2_P03363_2mut |
| EAAAKGGGGSEAAAK | 15,027 | SFVCP_Q87040 |
| GGSEAAAKGGG | 15,028 | FFV_O93209-Pro_2mutA |
| GSSPAPGGS | 15,029 | FOAMV_P14350-Pro_2mut |
| GGGGGGG | 15,030 | MMTVB_P03365-Pro |
| EAAAKGSS | 15,031 | SFV3L_P27401_2mutA |
| EAAAKGGGGSEAAAK | 15,032 | MMTVB_P03365-Pro |
| GGGGSEAAAKGGGGS | 15,033 | SFV1_P23074-Pro_2mutA |
| EAAAKGGGGSS | 15,034 | MMTVB_P03365 |
| GGGEAAAKGGS | 15,035 | SFV1_P23074 |
| PAPEAAAKGGG | 15,036 | MLVF5_P26810 |
| GGGGSSGGS | 15,037 | MMTVB_P03365 |
| GGSGSS | 15,038 | MMTVB_P03365 |
| PAPAPAPAPAPAP | 15,039 | KORV_Q9TTC1 |
| EAAAKGGG | 15,040 | SFV1_P23074-Pro_2mut |
| PAPAPAPAPAPAP | 15,041 | SRV2_P51517 |
| GSSGSSGSSGSSGSS | 15,042 | FFV_O93209-Pro_2mutA |
| GGGGSS | 15,043 | FOAMV_P14350_2mut |
| PAPGGGEAAAK | 15,044 | MMTVB_P03365_WS |
| GGSGGGEAAAK | 15,045 | FFV_O93209-Pro_2mut |
| PAPAPAPAPAP | 15,046 | MMTVB_P03365_WS |
| GGGEAAAKGGS | 15,047 | MMTVB_P03365-Pro |
| GGGEAAAKGSS | 15,048 | MMTVB_P03365_2mutB |
| GSSPAPEAAAK | 15,049 | MMTVB_P03365_WS |
| EAAAKEAAAKEAAAKEAAAKEAAAK | 15,050 | SFV1_P23074-Pro_2mutA |
| PAPGGG | 15,051 | SFV3L_P27401 |
| GSSEAAAKGGG | 15,052 | MMTVB_P03365_WS |
| GGGGSSEAAAK | 15,053 | FOAMV_P14350_2mut |
| PAPGSSGGS | 15,054 | SFV1_P23074-Pro_2mut |
| GSSGSSGSSGSSGSSGSS | 15,055 | SFV3L_P27401 |
| EAAAKGSSGGG | 15,056 | MMTVB_P03365 |
| PAPGGGGSS | 15,057 | WDSV_O92815_2mutA |
| GGSPAP | 15,058 | MMTVB_P03365-Pro |
| GGSGGSGGSGGSGGS | 15,059 | SFVCP_Q87040-Pro_2mut |
| PAPAPAPAP | 15,060 | MMTVB_P03365-Pro |
| GGGGG | 15,061 | HTL1A_P03362 |
| GGSGGSGGSGGS | 15,062 | SFV1_P23074_2mutA |
| GSSGSSGSSGSSGSS | 15,063 | FOAMV_P14350-Pro_2mut |
| PAPGGSEAAAK | 15,064 | MMTVB_P03365_2mutB_WS |
| PAPAPAPAP | 15,065 | SFV1_P23074_2mut |
| PAPGGGGSS | 15,066 | MMTVB_P03365 |
| GGSGSS | 15,067 | SFV3L_P27401_2mut |
| EAAAKEAAAKEAAAKEAAAK | 15,068 | MMTVB_P03365_2mut |
| EAAAKGGSGGG | 15,069 | HTL3P_Q4U0X6_2mut |
| PAPGGGGSS | 15,070 | SFVCP_Q87040-Pro_2mutA |
| EAAAKGGGGGS | 15,071 | MLVAV_P03356 |
| GGGGGS | 15,072 | FOAMV_P14350_2mut |
| GGGEAAAKGGS | 15,073 | FFV_O93209-Pro_2mutA |
| EAAAKPAPGGG | 15,074 | MMTVB_P03365_2mutB |
| GGSGGGPAP | 15,075 | FFV_093209_2mut |
| GSSEAAAKPAP | 15,076 | MMTVB_P03365 |
| PAPAPAPAPAPAP | 15,077 | SFV1_P23074_2mut |
| GGSPAPGGG | 15,078 | MMTVB_P03365-Pro |
| GGSGGGEAAAK | 15,079 | MMTVB_P03365 |
| PAPAP | 15,080 | SFVCP_Q87040 |
| GSSEAAAK | 15,081 | SFVCP_Q87040 |
| GGGGSGGGGSGGGGS | 15,082 | MMTVB_P03365-Pro |
| GSSGSSGSS | 15,083 | SFV3L_P27401 |
| EAAAKGGSGGG | 15,084 | MMTVB_P03365-Pro |
| GSSPAP | 15,085 | SFV1_P23074_2mut |
| GGGEAAAK | 15,086 | SFV1_P23074-Pro |
| AEAAAKEAAAKEAAAKEAAAKALEAEAAAKEAAAKEAAAKEAAAKA | 15,087 | MMTVB_P03365-Pro |
| PAPGGS | 15,088 | HTL1C_P14078_2mut |
| PAPGSSGGS | 15,089 | SFV1_P23074_2mut |
| PAPEAAAK | 15,090 | MMTVB_P03365_WS |
| PAPAP | 15,091 | MMTVB_P03365-Pro |
| EAAAKGGS | 15,092 | HTL1A_P03362_2mut |
| GGGGSEAAAKGGGGS | 15,093 | HTL1C_P14078 |
| EAAAKGSSGGS | 15,094 | FOAMV_P14350-Pro |
| PAPGGSGSS | 15,095 | MMTVB_P03365-Pro |
| PAPGGSEAAAK | 15,096 | SFV1_P23074_2mut |
| PAPGSSEAAAK | 15,097 | FFV_O93209-Pro_2mut |
| PAPGSSGGG | 15,098 | FOAMV_P14350-Pro_2mutA |
| GSSGGGEAAAK | 15,099 | AVIRE_P03360 |
| GGGGGG | 15,100 | SMRVH_P03364_2mut |
| PAPEAAAKGGG | 15,101 | MMTVB_P03365-Pro |
| GGGEAAAKGGS | 15,102 | SFVCP_Q87040_2mutA |
| PAPAPAPAPAP | 15,103 | SRV2_P51517 |
| GSSGSSGSSGSSGSSGSS | 15,104 | MMTVB_P03365 |
| EAAAKGGGPAP | 15,105 | MLVAV_P03356 |
| PAPAPAPAPAP | 15,106 | FOAMV_P14350-Pro_2mutA |
| PAPGGSEAAAK | 15,107 | FOAMV_P14350 |
| GSSGGGPAP | 15,108 | HTL32_Q0R5R2_2mutB |
| GGGGGSPAP | 15,109 | HTL3P_Q4U0X6_2mutB |
| GSSGGSGGG | 15,110 | MMTVB_P03365-Pro |
| PAPAP | 15,111 | SFVCP_Q87040-Pro |
| GSSGGGPAP | 15,112 | MMTVB_P03365-Pro |
| GGSGSS | 15,113 | MMTVB_P03365-Pro_2mut |
| GGSPAPEAAAK | 15,114 | SFV1_P23074-Pro_2mut |
| EAAAKGGSGGG | 15,115 | SFV3L_P27401_2mut |
| GGGGSSEAAAK | 15,116 | MMTVB_P03365_WS |
| GGGGGSGSS | 15,117 | MMTVB_P03365_2mut |
| GGGGSSGGS | 15,118 | SFV1_P23074-Pro_2mutA |
| EAAAKGGGGSEAAAK | 15,119 | MMTVB_P03365_WS |
| PAPGGGEAAAK | 15,120 | SFV1_P23074-Pro |
| PAPEAAAKGGG | 15,121 | MMTVB_P03365 |
| AEAAAKEAAAKEAAAKEAAAKALEAEAAAKEAAAKEAAAKEAAAKA | 15,122 | MMTVB_P03365 |
| GSSGGSEAAAK | 15,123 | FOAMV_P14350-Pro_2mut |
| GGSPAP | 15,124 | MLVBM_Q7SVK7_3mut |
| GSSEAAAK | 15,125 | FOAMV_P14350 |
| GSSEAAAK | 15,126 | MMTVB_P03365-Pro |
| EAAAKGSSGGS | 15,127 | HTL1A_P03362_2mut |
| GGGEAAAKPAP | 15,128 | FOAMV_P14350-Pro_2mut |
| EAAAKGGSPAP | 15,129 | FOAMV_P14350 |
| GSSEAAAKPAP | 15,130 | MMTVB_P03365_WS |
| GSSGSSGSS | 15,131 | FOAMV_P14350_2mut |
| EAAAKEAAAKEAAAKEAAAK | 15,132 | MMTVB_P03365_WS |
| EAAAK | 15,133 | MMTVB_P03365 |
| PAPGSS | 15,134 | BAEVM_P10272 |
| PAPGGS | 15,135 | FFV_093209-Pro_2mut |
| GGSGGS | 15,136 | SFV1_P23074-Pro_2mutA |
| SGGSSGGSSGSETPGTSESATPESSGGSSGGSS | 15,137 | HTLV2_P03363_2mut |
| GGSGGGEAAAK | 15,138 | MMTVB_P03365_WS |
| PAPGSSGGG | 15,139 | HTL1A_P03362 |
| GGSGGS | 15,140 | SFV3L_P27401-Pro |
| GSSGSS | 15,141 | SFV1_P23074-Pro |
| PAPGGSEAAAK | 15,142 | MMTVB_P03365 |
| GSAGSAAGSGEF | 15,143 | MMTVB_P03365-Pro |
| PAPGGG | 15,144 | FOAMV_P14350_2mut |
| EAAAKGGSGSS | 15,145 | MMTVB_P03365_WS |
| GSSGGGEAAAK | 15,146 | SFV3L_P27401-Pro |
| GGSGGGPAP | 15,147 | FOAMV_P14350-Pro_2mut |
| PAPAPAPAPAPAP | 15,148 | WDSV_O92815 |
| SGSETPGTSESATPES | 15,149 | SFVCP_Q87040-Pro_2mutA |
| GGSGGSGGS | 15,150 | SFV1_P23074 |
| GGGGSS | 15,151 | SFVCP_Q87040_2mut |
| GGGGGSEAAAK | 15,152 | MMTVB_P03365 |
| SGSETPGTSESATPES | 15,153 | MMTVB_P03365_WS |
| PAPAPAP | 15,154 | SFV3L_P27401 |
| PAPEAAAKGSS | 15,155 | MMTVB_P03365_2mutB_WS |
| GSSGSSGSSGSSGSS | 15,156 | SRV2_P51517 |
| GGGPAPGSS | 15,157 | HTL32_QOR5R2_2mutB |
| GGSGGGGSS | 15,158 | MMTVB_P03365-Pro |
| SGSETPGTSESATPES | 15,159 | SRV2_P51517 |
| EAAAKGSSGGS | 15,160 | MMTVB_P03365-Pro |
| GSSPAPEAAAK | 15,161 | MMTVB_P03365-Pro |
| GSSPAPEAAAK | 15,162 | SRV2_P51517 |
| GGGGSSPAP | 15,163 | MMTVB_P03365-Pro |
| PAPGGGEAAAK | 15,164 | SFV1_P23074-Pro_2mutA |
| PAPEAAAKGGS | 15,165 | MMTVB_P03365 |
| GSSGSSGSSGSSGSSGSS | 15,166 | FOAMV_P14350-Pro |
| GGSPAPGSS | 15,167 | SFV3L_P27401 |
| GGGPAPGGS | 15,168 | SFV1_P23074-Pro_2mutA |
| GGGPAPGSS | 15,169 | MMTVB_P03365-Pro |
| EAAAKPAP | 15,170 | MLVBM_Q7SVK7 |
| EAAAKEAAAKEAAAK | 15,171 | HTL1C_P14078 |
| GSSGGSEAAAK | 15,172 | SRV2_P51517 |
| PAPGGGGGS | 15,173 | SRV2_P51517 |
| GGGEAAAK | 15,174 | FFV_O93209-Pro_2mut |
| EAAAKGGGPAP | 15,175 | HTL32_QOR5R2 |
| GGSGSSGGG | 15,176 | MMTVB_P03365 |
| PAPEAAAKGSS | 15,177 | MMTVB_P03365-Pro |
| PAPGGGGGS | 15,178 | MMTVB_P03365-Pro |
| EAAAKGGGGGS | 15,179 | MMTVB_P03365_WS |
| GGGGGS | 15,180 | MMTVB_P03365-Pro |
| GGGGSGGGGSGGGGSGGGGSGGGGS | 15,181 | HTL1C_P14078 |
| EAAAKGGSPAP | 15,182 | MMTVB_P03365 |
| GGGGSSPAP | 15,183 | FFV_O93209-Pro_2mut |
| GGGGSSGGS | 15,184 | MMTVB_P03365-Pro |
| PAPGSSGGS | 15,185 | MMTVB_P03365-Pro |
| GGGGGS | 15,186 | SRV2_P51517 |
| GGSGSSGGG | 15,187 | MMTVB_P03365 |
| GSSGGSEAAAK | 15,188 | MMTVB_P03365-Pro |
| EAAAKEAAAKEAAAKEAAAK | 15,189 | GALV_P21414 |
| GGSEAAAKGGG | 15,190 | MMTVB_P03365-Pro |
| SGGSSGGSSGSETPGTSESATPESSGGSSGGSS | 15,191 | MMTVB_P03365-Pro |
| GSSEAAAKGGS | 15,192 | MMTVB_P03365 |
| GGGGSGGGGSGGGGSGGGGSGGGGSGGGGS | 15,193 | HTL3P_Q4U0X6_2mutB |
| GGGEAAAK | 15,194 | MMTVB_P03365-Pro |
| PAPAPAPAP | 15,195 | MMTVB_P03365-Pro |
| PAPGSSGGG | 15,196 | MMTVB_P03365 |
| GSSGSSGSSGSSGSS | 15,197 | GALV_P21414 |
| GGSPAP | 15,198 | MMTVB_P03365_WS |
| GGGGSGGGGSGGGGSGGGGSGGGGSGGGGS | 15,199 | MMTVB_P03365-Pro |
| PAPEAAAK | 15,200 | MMTVB_P03365-Pro |
| PAPGSSGGG | 15,201 | SFV1_P23074-Pro_2mutA |
| GGGGGSEAAAK | 15,202 | MMTVB_P03365_2mutB_WS |
| PAPAPAPAPAP | 15,203 | MMTVB_P03365-Pro |
| EAAAKGGSGSS | 15,204 | MMTVB_P03365-Pro |
| EAAAKEAAAKEAAAKEAAAK | 15,205 | MLVRD_P11227_3mut |
| PAPAPAPAP | 15,206 | FOAMV_P14350_2mutA |
| GGGPAPGSS | 15,207 | SFVCP_Q87040_2mut |
| PAPEAAAKGSS | 15,208 | SFVCP_Q87040_2mut |
| GGSPAPGGG | 15,209 | MMTVB_P03365-Pro |
| GGGGSGGGGSGGGGSGGGGS | 15,210 | MMTVB_P03365 |
| EAAAKGGS | 15,211 | HTL3P_Q4U0X6_2mut |
| PAPGSSGGS | 15,212 | MMTVB_P03365_WS |
| GGGGSGGGGS | 15,213 | MMTVB_P03365 |
| GGSGGS | 15,214 | FOAMV_P14350 |
| EAAAKGGGGSEAAAK | 15,215 | SFVCP_Q87040-Pro_2mut |
| EAAAKEAAAKEAAAKEAAAK | 15,216 | MMTVB_P03365-Pro_2mutB |
| PAPGGGEAAAK | 15,217 | SFVCP_Q87040-Pro |
| GSSGSS | 15,218 | JSRV_P31623_2mutB |
| EAAAKGGGGGS | 15,219 | MMTVB_P03365_2mut_WS |
| GSSPAPEAAAK | 15,220 | MMTVB_P03365-Pro |
| GGGEAAAK | 15,221 | HTL1C_P14078 |
| PAPEAAAKGSS | 15,222 | HTL32_QOR5R2_2mutB |
| GGGGSSEAAAK | 15,223 | MMTVB_P03365-Pro |
| PAPGSSGGS | 15,224 | MMTVB_P03365-Pro |
| EAAAKGGGGGS | 15,225 | MMTVB_P03365 |
| GGGGGGGGSGGGGSGGGGS | 15,226 | MMTVB_P03365 |
| EAAAKGGGGSS | 15,227 | HTL3P_Q4U0X6_2mut |
| GGGEAAAKGGS | 15,228 | SFVCP_Q87040-Pro |
| GGGGGSPAP | 15,229 | MMTVB_P03365-Pro_2mutB |
| GGSGGGEAAAK | 15,230 | SFV3L_P27401-Pro |
| PAPGGGGGS | 15,231 | SFV3L_P27401-Pro |
| EAAAKGGGGSEAAAK | 15,232 | MMTVB_P03365 |
| PAPEAAAKGSS | 15,233 | MMTVB_P03365-Pro |
| GGSEAAAKGGG | 15,234 | MMTVB_P03365-Pro |
| GGSGGSGGSGGSGGS | 15,235 | SMRVH_P03364_2mutB |
| GGSGGSGGSGGSGGS | 15,236 | HTL1L_POC211_2mut |
| GGGGGG | 15,237 | WDSV_O92815 |
| GGGGGSGSS | 15,238 | MMTVB_P03365-Pro |
| GGSEAAAKPAP | 15,239 | SFV3L_P27401-Pro_2mut |
| GGGPAPGSS | 15,240 | MMTVB_P03365_2mut_WS |
| GGGGGS | 15,241 | MMTVB_P03365_WS |
| GGSPAPEAAAK | 15,242 | MMTVB_P03365 |
| PAPEAAAKGGS | 15,243 | HTL1A_P03362 |
| EAAAKGGSGSS | 15,244 | MMTVB_P03365_2mut_WS |
| GGGPAPEAAAK | 15,245 | SFV3L_P27401-Pro_2mut |
| PAPGGGGSS | 15,246 | HTL32_QOR5R2_2mut |
| GSSPAPGGG | 15,247 | HTL3P_Q4U0X6_2mut |
| GGGGSSGGS | 15,248 | BLVAU_P25059_2mut |
| EAAAKGGGGGS | 15,249 | HTL1L_POC211 |
| GGSEAAAKGSS | 15,250 | JSRV_P31623_2mutB |
| GSSGGG | 15,251 | JSRV_P31623 |
| GGSGGSGGSGGS | 15,252 | MMTVB_P03365-Pro |
| EAAAKPAP | 15,253 | SFV1_P23074-Pro_2mutA |
| GGGGSSGGS | 15,254 | MMTVB_P03365_WS |
| GGSGGS | 15,255 | MMTVB_P03365_WS |
| EAAAKGGGGGS | 15,256 | MMTVB_P03365-Pro |
| GGGGSGGGGSGGGGSGGGGSGGGGSGGGGS | 15,257 | MMTVB_P03365 |
| GGSGGSGGS | 15,258 | MMTVB_P03365 |
| GGGGGSEAAAK | 15,259 | MLVBM_Q7SVK7 |
| GGSGSSPAP | 15,260 | MMTVB_P03365_WS |
| EAAAKEAAAKEAAAK | 15,261 | JSRV_P31623 |
| PAPEAAAKGGS | 15,262 | MMTVB_P03365-Pro |
| GGSGSSEAAAK | 15,263 | FOAMV_P14350 |
| GGGGGSGSS | 15,264 | MMTVB_P03365-Pro_2mut |
| GGGPAPGGS | 15,265 | MMTVB_P03365 |
| SGSETPGTSESATPES | 15,266 | SFVCP_Q87040_2mut |
| GSSPAPGGS | 15,267 | SFV1_P23074-Pro_2mutA |
| GSSGSSGSSGSSGSS | 15,268 | MMTVB_P03365 |
| EAAAKGGGPAP | 15,269 | MMTVB_P03365 |
| GSSGGG | 15,270 | MMTVB_P03365_2mut_WS |
| GGGEAAAKPAP | 15,271 | MMTVB_P03365 |
| PAPGGSGGG | 15,272 | MMTVB_P03365-Pro |
| GSSGGSGGG | 15,273 | WDSV_O92815_2mut |
| GGSGGG | 15,274 | HTL32_Q0R5R2_2mut |
| EAAAKGGSPAP | 15,275 | HTLV2_P03363_2mut |
| GGSPAPEAAAK | 15,276 | MMTVB_P03365-Pro |
| GSSGGSEAAAK | 15,277 | MMTVB_P03365_2mut |
| GSAGSAAGSGEF | 15,278 | MMTVB_P03365_WS |
| PAPGGSGSS | 15,279 | FFV_O93209 |
| GGSEAAAKGGG | 15,280 | MMTVB_P03365 |
| GGSPAPGSS | 15,281 | MMTVB_P03365-Pro |
| GSSGGSGGG | 15,282 | SFV3L_P27401 |
| PAPEAAAKGGG | 15,283 | HTL1A_P03362_2mutB |
| GGGEAAAKPAP | 15,284 | MMTVB_P03365-Pro |
| GGSEAAAK | 15,285 | HTL32_Q0R5R2_2mutB |
| GGGEAAAKGSS | 15,286 | MPMV_P07572 |
| GGGGGSEAAAK | 15,287 | MMTVB_P03365-Pro |
| PAPAPAPAPAP | 15,288 | SFVCP_Q87040-Pro_2mutA |
| PAPAPAPAPAP | 15,289 | HTL1L_POC211_2mut |
| GGGGSSGGS | 15,290 | HTL3P_Q4U0X6 |
| PAPGGSEAAAK | 15,291 | MMTVB_P03365_2mut_WS |
| PAPAPAPAPAP | 15,292 | HTL1A_P03362 |
| EAAAKPAPGGG | 15,293 | MMTVB_P03365_2mut_WS |
| GGSEAAAK | 15,294 | MMTVB_P03365_2mut_WS |
| GGGEAAAKGSS | 15,295 | SFV1_P23074-Pro_2mutA |
| GGSPAPGSS | 15,296 | MMTVB_P03365-Pro |
| GGSEAAAKPAP | 15,297 | MLVBM_Q7SVK7 |
| PAPEAAAKGGG | 15,298 | MMTVB_P03365_2mut_WS |
| GSSEAAAKPAP | 15,299 | MMTVB_P03365-Pro_2mutB |
| GGGGSEAAAKGGGGS | 15,300 | MMTVB_P03365-Pro_2mut |
| GSSEAAAKGGS | 15,301 | MMTVB_P03365-Pro_2mutB |
| GSSGSSGSSGSSGSS | 15,302 | SRV2_P51517_2mutB |
| GGGGGSPAP | 15,303 | HTL1L_POC211_2mut |
| GGSEAAAK | 15,304 | MMTVB_P03365 |
| GSSPAPEAAAK | 15,305 | SMRVH_P03364_2mutB |
| GGGPAPGGS | 15,306 | HTL1C_P14078_2mut |
| GGSPAPEAAAK | 15,307 | MMTVB_P03365_WS |
| GGSEAAAKPAP | 15,308 | HTL1A_P03362_2mut |
| PAPAPAPAP | 15,309 | HTLV2_P03363_2mut |
| GSSPAPGGG | 15,310 | MMTVB_P03365 |
| GSSGSSGSSGSS | 15,311 | MMTVB_P03365-Pro |
| GGSEAAAKGSS | 15,312 | MMTVB_P03365_WS |
| GGSGSSGGG | 15,313 | MMTVB_P03365_2mutB |
| GSSGSSGSSGSSGSSGSS | 15,314 | JSRV_P31623_2mutB |
| GGSEAAAKPAP | 15,315 | MMTVB_P03365-Pro |
| GSSGGSGGG | 15,316 | HTLV2_P03363_2mut |
| AEAAAKEAAAKEAAAKEAAAKALEAEAAAKEAAAKEAAAKEAAAKA | 15,317 | WDSV_O92815_2mut |
| GGSPAPEAAAK | 15,318 | MMTVB_P03365 |
| GGGGSSEAAAK | 15,319 | MMTVB_P03365 |
| GGSGGGEAAAK | 15,320 | SFV1_P23074-Pro_2mutA |
| GGGGSEAAAKGGGGS | 15,321 | WDSV_O92815_2mut |
| GGSGSSEAAAK | 15,322 | MMTVB_P03365_2mutB_WS |
| GGSEAAAKPAP | 15,323 | MMTVB_P03365_WS |
| GSSGGGEAAAK | 15,324 | SFVCP_Q87040-Pro |
| GSSGGS | 15,325 | SFVCP_Q87040-Pro_2mut |
| GGSEAAAKPAP | 15,326 | SFVCP_Q87040_2mut |
| GSSGGSEAAAK | 15,327 | SFVCP_Q87040_2mut |
| GSSPAPEAAAK | 15,328 | SRV2_P51517_2mutB |
| GGSGGSGGSGGSGGSGGS | 15,329 | BLVAU_P25059 |
| GSSGSSGSSGSSGSS | 15,330 | HTL1C_P14078_2mut |
| EAAAKGGGGSS | 15,331 | MMTVB_P03365_2mutB |
| GGGEAAAKGSS | 15,332 | SFVCP_Q87040-Pro |

### Example 16: Quantifying Activity of a Gene Editing Polypeptide and Template for Rewriting the Endogenous FAH Locus Achieved in Primary Mouse Hepatocytes

This example demonstrates the use of a heterologous gene modifying system containing a heterologous gene modifying polypeptide and a template RNA, to convert an A nucleotide to a G nucleotide in the endogenous Fah locus in mouse primary hepatocytes derived from a Fah5981SB mouse. The Fah5981SB mouse model harbors a G to A point mutation in the last nucleotide of exon 8 of the Fah gene, leading to aberrant mRNA splicing and subsequent mRNA degradation, without the production of Fah protein and, and thus serves as a mouse model of hereditary tyrosinemia type I.

In this example, the template RNA contained:
(1) a gRNA spacer;
(2) a gRNA scaffold;
(3) a heterologous object sequence; and
(4) a primer binding site (PBS) sequence.

More specifically, the template RNA (including chemical modification pattern) comprised the following sequences:

FAH1_R14_P12_Heavy RNACS048-001
(SEQ ID NO: 30421)
mG*mG*mA*rUrGrGrUrCrCrUrCrArUrGrArArCrGrArCrGrUrUr
UrUrArGrAmGmCmUmAmGmAmAmAmUmAmGmCrArArGrUrUrArArAr
ArUrArArGrGrCrUrArGrUrCrCrGrUrUrArUrCrAmAmCmUmUmGm
AmAmAmAmAmGmUmGmGmCmAmCmCmGmAmGmUmCmGmGmUmGmCrUrUr
ArCrCrGrCrUrCrCrArGrUrCrGrUrUrCrArUrGrArG*mG*mA*mC
FAH1_R15_P10_Heavy RNACS049-001
(SEQ ID NO: 30422)
mG*mG*mA*rUrGrGrUrCrCrUrCrArUrGrArArCrGrArCrGrUrUr
UrUrArGrAmGmCmUmAmGmAmAmAmUmAmGmCrArArGrUrUrArArAr
ArUrArArGrGrCrUrArGrUrCrCrGrUrUrArUrCrAmAmCmUmUmGm
AmAmAmAmAmGmUmGmGmCmAmCmCmGmAmGmUmCmGmGmUmGmCrArUr
UrArCrCrGrCrUrCrCrArGrUrCrGrUrUrCrArUrG*mA*mG*Mg
FAH2_R19_P11_MUT_Heavy RNACS052-001
(SEQ ID NO: 30423)
mU*mC*mA*rGrArGrGrArArGrCrUrGrGrGrCrCrArCrCrGrUrUr
UrUrArGrAmGmCmUmAmGmAmAmAmUmAmGmCrArArGrUrUrArArAr
ArUrArArGrGrCrUrArGrUrCrCrGrUrUrArUrCrAmAmCmUmUmGm
AmAmAmAmAmGmUmGmGmCmAmCmCmGmAmGmUmCmGmGmUmGmCrUrGr
GrArGrCrGrGrUrArArUrGrGrCrUrGrGrUrGrGrCrCrCrArGrC*
mU*mU*mC
FAH2_R19_P13_MUT_Heavy RNACS053-001
(SEQ ID NO: 30424)
mU*mC*mA*rGrArGrGrArArGrCrUrGrGrGrCrCrArCrCrGrUrUr
UrUrArGrAmGmCmUmAmGmAmAmAmUmAmGmCrArArGrUrUrArArAr
ArUrArArGrGrCrUrArGrUrCrCrGrUrUrArUrCrAmAmCmUmUmGm
AmAmAmAmAmGmUmGmGmCmAmCmCmGmAmGmUmCmGmGmUmGmCrUrGr
GrArGrCrGrGrUrArArUrGrGrCrUrGrGrUrGrGrCrCrCrArGrCr
UrU*mC*mC*mU

Additional exemplary template RNAs that could be utilized in this experiment include the following:

FAH1 RNACS050
(SEQ ID NO: 30425)
mG*mG*mA*rUrGrGrUrCrCrUrCrArUrGrArArCrGrArCrGrUrUr
UrUrArGrAmGmCmUmAmGmAmAmAmUmAmGmCrArArGrUrUrArArAr
ArUrArArGrGrCrUrArGrUrCrCrGrUrUrArUrCrAmAmCmUmUmGm
AmAmAmAmAmGmUmGmGmCmAmCmCmGmAmGmUmCmGmGmUmGmCrArGr
GrCrArUrUrArCrCrGrCrUrCrCrArGrUrCrGrUrUrCrArUrGrAr
G*mG*mA*mC
FAH1 RNACS051
(SEQ ID NO: 30426)
mG*mG*mA*rUrGrGrUrCrCrUrCrArUrGrArArCrGrArCrGrUrUr
UrUrArGrAmGmCmUmAmGmAmAmAmUmAmGmCrArArGrUrUrArArAr
ArUrArArGrGrCrUrArGrUrCrCrGrUrUrArUrCrAmAmCmUmUmGm
AmAmAmAmAmGmUmGmGmCmAmCmCmGmAmGmUmCmGmGmUmGmCrArGr
GrCrArUrUrArCrCrGrCrUrCrCrArGrUrCrGrUrUrCrArUrG*mA
*mG*mG
In the sequences above m=2′-O-methyl
ribonucleotide, r=ribose and *= phosphorothioate
bond.

The heterologous gene modifying polypeptides tested comprised sequence of: RNAV209 (nCas9-RT) and RNAV214 (wtCas9-RT). Specifically, the nCas9-RT and the wtCas9-RT had the following amino acid sequences:

nCas9-RT (RNAV209):
(SEQ ID NO: 30427)
MPAAKRVKLDGGDKKYSIGLDIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVE
EDKKHERHPIFGNIVDEVAYHEKYPTIYHLRKKLVDSTDKADLRLIYLALAHMIKFRGHF
LIEGDLNPDNSDVDKLFIQLVQTYNQLFEENPINASGVDAKAILSARLSKSRRLENLIAQL
PGEKKNGLFGNLIALSLGLTPNFKSNFDLAEDAKLQLSKDTYDDDLDNLLAQIGDQYAD
LFLAAKNLSDAILLSDILRVNTEITKAPLSASMIKRYDEHHQDLTLLKALVRQQLPEKYK
EIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKS
EETITPWNFEEVVDKGASAQSFIERMTNFDKNLPNEKVLPKHSLLYEYFTVYNELTKVK
YVTEGMRKPAFLSGEQKKAIVDLLFKTNRKVTVKQLKEDYFKKIECFDSVEISGVEDRF
NASLGTYHDLLKIIKDKDFLDNEENEDILEDIVLTLTLFEDREMIEERLKTYAHLFDDKV
MKQLKRRRYTGWGRLSRKLINGIRDKQSGKTILDFLKSDGFANRNFMQLIHDDSLTFKE
DIQKAQVSGQGDSLHEHIANLAGSPAIKKGILQTVKVVDELVKVMGRHKPENIVIEMAR
ENQTTQKGQKNSRERMKRIEEGIKELGSQILKEHPVENTQLQNEKLYLYYLQNGRDMY
VDQELDINRLSDYDVDHIVPQSFLKDDSIDNKVLTRSDKARGKSDNVPSEEVVKKMKN
YWRQLLNAKLITQRKFDNLTKAERGGLSELDKAGFIKRQLVETRQITKHVAQILDSRMN
TKYDENDKLIREVKVITLKSKLVSDFRKDFQFYKVREINNYHHAHDAYLNAVVGTALIK
KYPKLESEFVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESILPKRNSDKL
IARKKDWDPKKYGGFDSPTVAYSVLVVAKVEKGKSKKLKSVKELLGITIMERSSFEKNP
IDFLEAKGYKEVKKDLIIKLPKYSLFELENGRKRMLASAGELQKGNELALPSKYVNFLYL
ASHYEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGLYETRI
DLSQLGGDSGGSSGGSSGSETPGTSESATPESSGGSSGGSSTLNIEDEYRLHETSKEPDVS
LGSTWLSDFPQAWAETGGMGLAVRQAPLIIPLKATSTPVSIKQYPMSQEARLGIKPHIQR
LLDQGILVPCQSPWNTPLLPVKKPGTNDYRPVQDLREVNKRVEDIHPTVPNPYNLLSGL
PPSHQWYTVLDLKDAFFCLRLHPTSQPLFAFEWRDPEMGISGQLTWTRLPQGFKNSPTL
FNEALHRDLADFRIQHPDLILLQYVDDLLLAATSELDCQQGTRALLQTLGNLGYRASAK
KAQICQKQVKYLGYLLKEGQRWLTEARKETVMGQPTPKTPRQLREFLGKAGFCRLFIPG
FAEMAAPLYPLTKPGTLFNWGPDQQKAYQEIKQALLTAPALGLPDLTKPFELFVDEKQG
YAKGVLTQKLGPWRRPVAYLSKKLDPVAAGWPPCLRMVAAIAVLTKDAGKLTMGQPL
VILAPHAVEALVKQPPDRWLSNARMTHYQALLLDTDRVQFGPVVALNPATLLPLPEEG
LOHNCLDILAEAHGTRPDLTDQPLPDADHTWYTDGSSLLQEGQRKAGAAVTTETEVIW
AKALPAGTSAQRAELIALTQALKMAEGKKLNVYTDSRYAFATAHIHGEIYRRRGWLTS
EGKEIKNKDEILALLKALFLPKRLSIIHCPGHQKGHSAEARGNRMADQAARKAAITETPD
TSTLLIENSSPSGGSKRTADGSEFEKRTADGSEFESPKKKAKVE
wtCas9-RT (RNAV214-040):
(SEQ ID NO: 30428)
MPAAKRVKLDGGDKKYSIGLDIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVE
EDKKHERHPIFGNIVDEVAYHEKYPTIYHLRKKLVDSTDKADLRLIYLALAHMIKFRGHF
LIEGDLNPDNSDVDKLFIQLVQTYNQLFEENPINASGVDAKAILSARLSKSRRLENLIAQL
PGEKKNGLFGNLIALSLGLTPNFKSNFDLAEDAKLQLSKDTYDDDLDNLLAQIGDQYAD
LFLAAKNLSDAILLSDILRVNTEITKAPLSASMIKRYDEHHQDLTLLKALVRQQLPEKYK
EIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKS
EETITPWNFEEVVDKGASAQSFIERMTNFDKNLPNEKVLPKHSLLYEYFTVYNELTKVK
YVTEGMRKPAFLSGEQKKAIVDLLFKTNRKVTVKQLKEDYFKKIECFDSVEISGVEDRF
NASLGTYHDLLKIIKDKDFLDNEENEDILEDIVLTLTLFEDREMIEERLKTYAHLFDDKV
MKQLKRRRYTGWGRLSRKLINGIRDKQSGKTILDFLKSDGFANRNFMQLIHDDSLTFKE
DIQKAQVSGQGDSLHEHIANLAGSPAIKKGILQTVKVVDELVKVMGRHKPENIVIEMAR
ENQTTQKGQKNSRERMKRIEEGIKELGSQILKEHPVENTQLQNEKLYLYYLQNGRDMY
VDQELDINRLSDYDVDHIVPQSFLKDDSIDNKVLTRSDKNRGKSDNVPSEEVVKKMKN
YWRQLLNAKLITQRKFDNLTKAERGGLSELDKAGFIKRQLVETRQITKHVAQILDSRMN
TKYDENDKLIREVKVITLKSKLVSDFRKDFQFYKVREINNYHHAHDAYLNAVVGTALIK
KYPKLESEFVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESILPKRNSDKL
IARKKDWDPKKYGGFDSPTVAYSVLVVAKVEKGKSKKLKSVKELLGITIMERSSFEKNP
IDFLEAKGYKEVKKDLIIKLPKYSLFELENGRKRMLASAGELQKGNELALPSKYVNFLYL
ASHYEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGLYETRI
DLSQLGGDSGGSSGGSSGSETPGTSESATPESSGGSSGGSSTLNIEDEYRLHETSKEPDVS
LGSTWLSDFPQAWAETGGMGLAVRQAPLIIPLKATSTPVSIKQYPMSQEARLGIKPHIQR
LLDQGILVPCQSPWNTPLLPVKKPGTNDYRPVQDLREVNKRVEDIHPTVPNPYNLLSGL
PPSHQWYTVLDLKDAFFCLRLHPTSQPLFAFEWRDPEMGISGQLTWTRLPQGFKNSPTL
FNEALHRDLADFRIQHPDLILLQYVDDLLLAATSELDCQQGTRALLQTLGNLGYRASAK
KAQICQKQVKYLGYLLKEGQRWLTEARKETVMGQPTPKTPRQLREFLGKAGFCRLFIPG
FAEMAAPLYPLTKPGTLFNWGPDQQKAYQEIKQALLTAPALGLPDLTKPFELFVDEKQG
YAKGVLTQKLGPWRRPVAYLSKKLDPVAAGWPPCLRMVAAIAVLTKDAGKLTMGQPL
VILAPHAVEALVKQPPDRWLSNARMTHYQALLLDTDRVQFGPVVALNPATLLPLPEEG
LOHNCLDILAEAHGTRPDLTDQPLPDADHTWYTDGSSLLQEGQRKAGAAVTTETEVIW
AKALPAGTSAQRAELIALTQALKMAEGKKLNVYTDSRYAFATAHIHGEIYRRRGWLTS
EGKEIKNKDEILALLKALFLPKRLSIIHCPGHQKGHSAEARGNRMADQAARKAAITETPD
TSTLLIENSSPSGGSKRTADGSEFEKRTADGSEFESPKKKAKVE
Underlining indicates the residue that differs between the
nickase and wild-type sequences.

The heterologous gene modifying system comprising the heterologous gene modifying polypeptides listed above and the template RNA described above were transfected into primary mouse hepatocytes. The heterologous gene modifying polypeptide and the template RNA were delivered by nucleofection in the RNA format. Specifically, 4 μg of heterologous gene modifying polypeptide mRNA were combined with 10 μg of chemically synthesized template RNA in 5 μL of water. The transfection mix was added to 100,000 mouse primary hepatocytes in Buffer P3 [Lonza], and cells were nucleofected using program DG-138. After nucleofection, cells were grown at 37° C., 5% CO₂ for 3 days prior to cell lysis and genomic DNA extraction. To analyze gene editing activity, primers flanking the target insertion site locus were used to amplify across the locus. Amplicons were analyzed via short read sequencing using an Illumina MiSeq. Conversion of terminal A to G sequence in exon 8 of fah gene indicates successful editing.

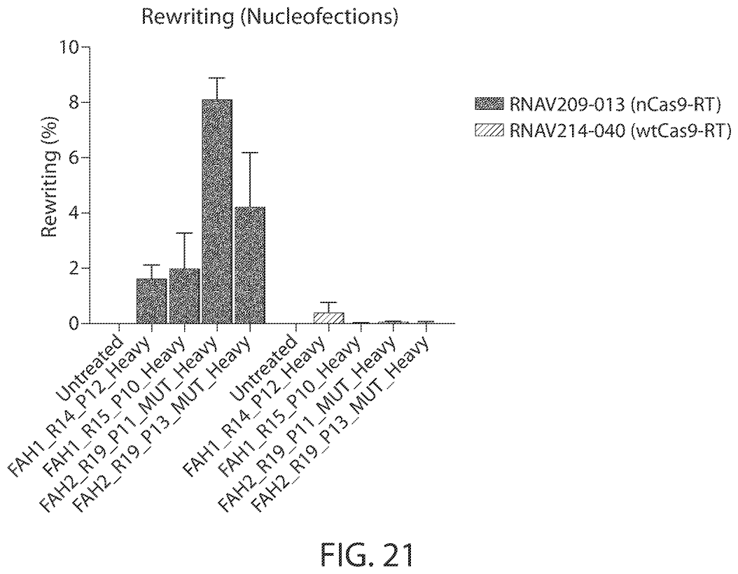

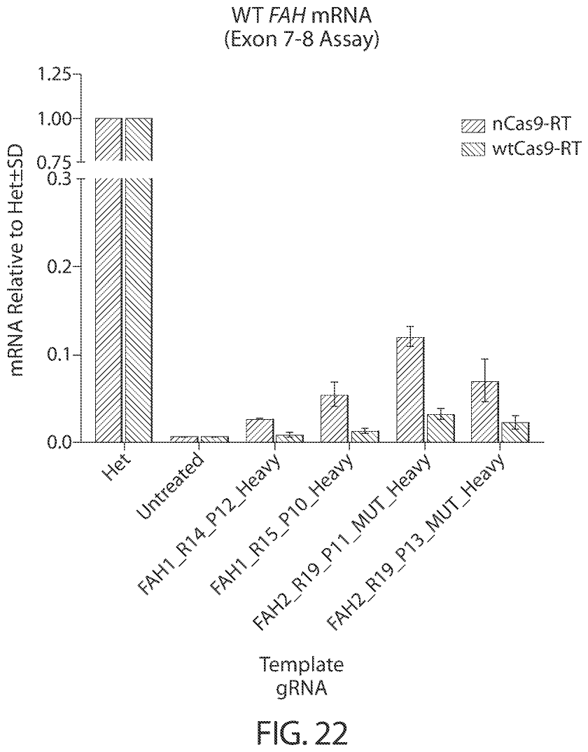

As shown in FIG. 21, for FAH2 templates, perfect rewrite levels (conversion of A to G with no unwanted mutations detected) of 4-8% were detected with RNAV209 but not with RNAV214. Indel levels of 4.4 to 6.6% were observed with RNAV209. Furthermore, the amount of WT Fah mRNA was measured using quantitative RT-PCR using primers that bind to exons 7 and 8. As shown in FIG. 22, FAH2 templates result in an increase in the abundance of Fah mRNA relative to WT by up to 12% when FAH2 template is tested with RNAV209 mRNA. These results demonstrate the use of a heterologous gene modifying system to reverse a mutation in the Fah gene, resulting in partial restoration of the expression of wild-type Fah mRNA.

### Example 17: Quantifying Activity of a Gene Editing Polypeptide and Template In Vivo for Rewriting the Endogenous FAH Locus Achieved in Mouse Liver

This example demonstrates the use of a heterologous gene modifying system containing a heterologous gene modifying polypeptide and a template RNA, to convert an A nucleotide to a G nucleotide in the Fah5981SB mouse model into the endogenous Fah locus in mouse liver. The Fah5981SB mouse model harbors a G to A point mutation in the last nucleotide of exon 8 of the Fah gene, leading to aberrant mRNA splicing and subsequent mRNA degradation, without the production of Fah protein and serves as a mouse model of hereditary tyrosinemia type I.

In this example, the template RNA contained:
(1) a gRNA spacer;
(2) a gRNA scaffold;
(3) a heterologous object sequence; and
(4) a primer binding site (PBS) sequence.

More specifically, the template RNA comprised the following sequences:

FAH1_R14_P12_Heavy RNACS048-001
(SEQ ID NO: 30429)
mG*mG*mA*rUrGrGrUrCrCrUrCrArUrGrArArCrGrArCrGrUrUr
UrUrArGrAmGmCmUmAmGmAmAmAmUmAmGmCrArArGrUrUrArArAr
ArUrArArGrGrCrUrArGrUrCrCrGrUrUrArUrCrAmAmCmUmUmGm
AmAmAmAmAmGmUmGmGmCmAmCmCmGmAmGmUmCmGmGmUmGmCrUrUr
ArCrCrGrCrUrCrCrArGrUrCrGrUrUrCrArUrGrArG*mG*mA*mC
FAH1_R15_P10_Heavy RNACS049-001
(SEQ ID NO: 30430)
mG*mG*mA*rUrGrGrUrCrCrUrCrArUrGrArArCrGrArCrGrUrUr
UrUrArGrAmGmCmUmAmGmAmAmAmUmAmGmCrArArGrUrUrArArAr
ArUrArArGrGrCrUrArGrUrCrCrGrUrUrArUrCrAmAmCmUmUmGm
AmAmAmAmAmGmUmGmGmCmAmCmCmGmAmGmUmCmGmGmUmGmCrArUr
UrArCrCrGrCrUrCrCrArGrUrCrGrUrUrCrArUrG*mA*mG*mG
FAH2_R19_P11_MUT_Heavy RNACS052-001
(SEQ ID NO:30431)
mU*mC*mA*rGrArGrGrArArGrCrUrGrGrGrCrCrArCrCrGrUrUr
UrUrArGrAmGmCmUmAmGmAmAmAmUmAmGmCrArArGrUrUrArArAr
ArUrArArGrGrCrUrArGrUrCrCrGrUrUrArUrCrAmAmCmUmUmGm
AmAmAmAmAmGmUmGmGmCmAmCmCmGmAmGmUmCmGmGmUmGmCrUrGr
GrArGrCrGrGrUrArArUrGrGrCrUrGrGrUrGrGrCrCrCrArGrC*
mU*mU*mC
FAH2_R19_P13_MUT_Heavy RNACS053-001
(SEQ ID NO: 30432)
mU*mC*mA*rGrArGrGrArArGrCrUrGrGrGrCrCrArCrCrGrUrUr
UrUrArGrAmGmCmUmAmGmAmAmAmUmAmGmCrArArGrUrUrArArAr
ArUrArArGrGrCrUrArGrUrCrCrGrUrUrArUrCrAmAmCmUmUmGm
AmAmAmAmAmGmUmGmGmCmAmCmCmGmAmGmUmCmGmGmUmGmCrUrGr
GrArGrCrGrGrUrArArUrGrGrCrUrGrGrUrGrGrCrCrCrArGrCr
UrU*mC*mC*mU

The heterologous gene modifying polypeptides tested comprised a sequence of: RNAV209 and RNAV214, the sequences of which are each provided in Example 16.

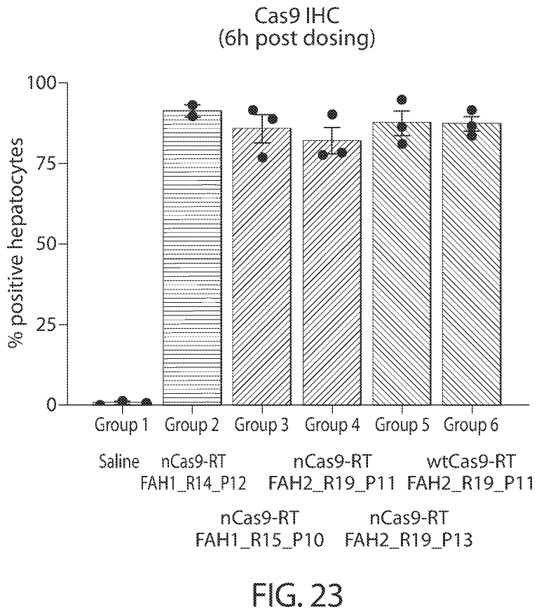

The heterologous gene modifying system comprising the heterologous gene modifying polypeptides and the template RNA described above was formulated in LNP and delivered to mice. Specifically, 2 mg/kg of total RNA equivalent formulated in LNPs, combined at 1:1 (w/w) of template RNA and mRNA, were dosed intravenously in 7 to 9-week-old, mixed gender Fah5981SB mice. Six hours or 6 days post-dosing, animals were sacrificed, and their liver collected for analyses. To determine the expression distribution of the heterologous gene modifying polypeptide in the liver, 6-hr liver samples were subjected to immunohistochemistry using an anti-Cas9 antibody. Upon staining, quantification of Cas9-positive hepatocytes was determined by QuPath Markup. As shown in FIG. 23, the expression of the heterologous gene modifying polypeptide was observed in 82-91% of hepatocytes.

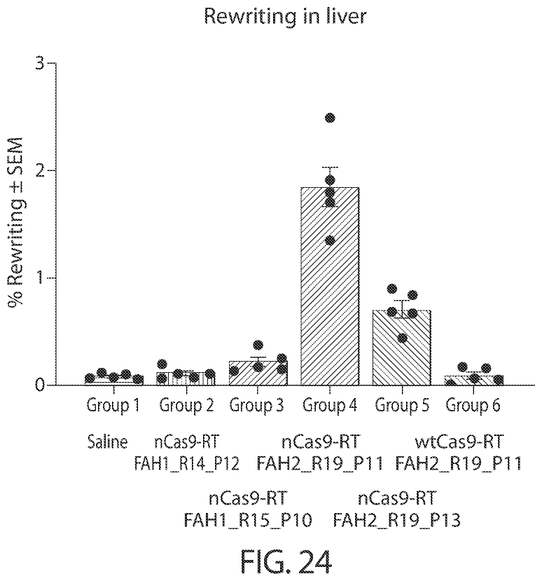

To analyze gene editing activity, primers flanking the target insertion site locus were used to amplify across the locus in the genomic DNA of liver samples collected 6 days post-dosing. Amplicons were analyzed via short read sequencing using an Illumina MiSeq. Conversion of an A nucleotide to a G nucleotide indicates successful editing. As shown in FIG. 24, perfect rewrite levels (conversion of A to G with no unwanted mutations detected) of 0.1%-1.9% were detected across the different groups. Indel levels were in the range of 0.2%-0.4%.

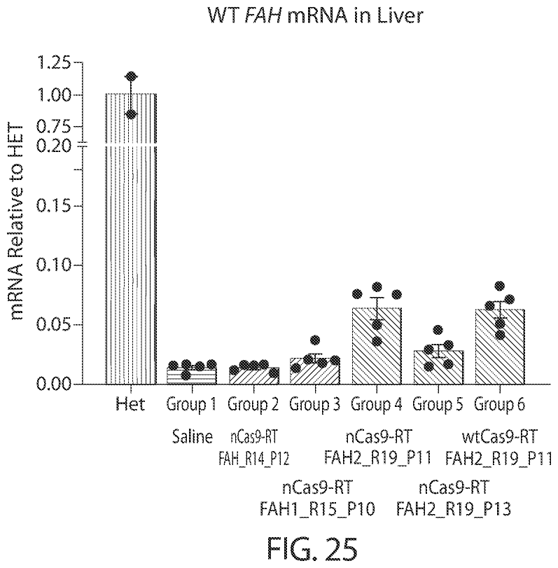

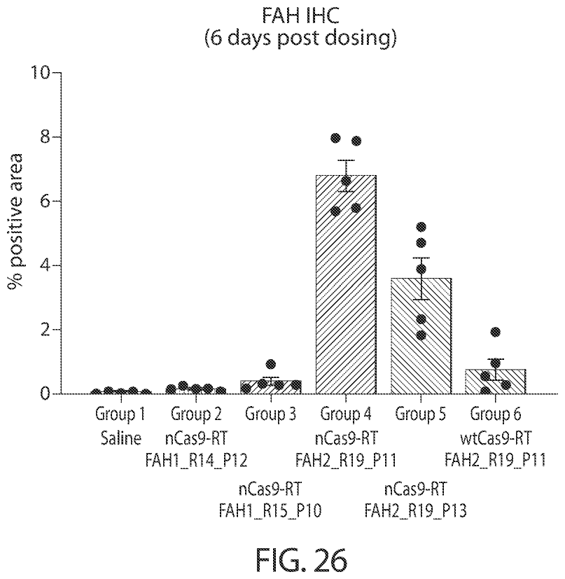

To determine the phenotypic correction caused by the gene editing activity, the restoration of wild-type FAH mRNA was determined by real-time qRT-PCR, and the restoration of Fah protein expression determined by immunohistochemistry using an anti-Fah antibody. As shown in FIG. 25, wild-type mRNA restoration of 0.1%-6%, relative to littermate heterozygous mice, was detected across the different groups. As shown in FIG. 26, Fah protein was detected in 0.1%-7% of liver cross-sectional area across the different groups. These results demonstrate the use of a heterologous gene modifying system to reverse a mutation in the Fah gene in an in vivo mouse model for hereditary tyrosinemia type I, resulting in partial restoration of expression of wild-type Fah mRNA and Fah protein.

### Example 18. Gene Editing at the TTR Locus in an In Vivo Mouse Model

This Example demonstrates successful delivery of an mRNA and guide using Cas9-mediated gene editing using the protospacer sequence ACACAAAUACCAGUCCAGCG (SEQ ID NO: 37641) that targets the TTR locus using a heterologous gene modifying polypeptide and RNA in a C57Blk/6 mouse.

RNAs were prepared as follows. An mRNA encoding a heterologous gene modifying polypeptide having the sequence shown in Table 5A1 below was produced by in vitro transcription and the purified mRNA was dissolved in 1 mM sodium citrate, pH 6, to a final concentration of RNA of 1-2 mg/mL. Similarly, a guide RNA having a sequence shown in Table 5A1 below was produced by chemical synthesis and dissolved in water or aqueous buffer, to a final concentration of RNA of 1-2 mg/mL.

TABLE 5A1
Sequences of Example 18

| | SEQ | |
|---|---|---|
| | ID | |
| Name | NO | Nucleic acid sequence |
| Cas9-RT | 30433 | AUGCCUGCGGCUAAGCGGGUAAAAUUGGAUGGUGGGGACAAG |
| gene | | AAGUACAGCAUCGGCCUGGACAUCGGCACCAACUCUGUGGGCU |
| modifying | | GGGCCGUGAUCACCGACGAGUACAAGGUGCCCAGCAAGAAAUU |
| polypeptide | | CAAGGUGCUGGGCAACACCGACCGGCACAGCAUCAAGAAGAAC |
| | | CUGAUCGGAGCCCUGCUGUUCGACAGCGGCGAAACAGCCGAGG |
| | | CCACCCGGCUGAAGAGAACCGCCAGAAGAAGAUACACCAGACG |
| | | GAAGAACCGGAUCUGCUAUCUGCAAGAGAUCUUCAGCAACGAG |
| | | AUGGCCAAGGUGGACGACAGCUUCUUCCACAGACUGGAAGAGU |
| | | CCUUCCUGGUGGAAGAGGAUAAGAAGCACGAGCGGCACCCCAU |
| | | CUUCGGCAACAUCGUGGACGAGGUGGCCUACCACGAGAAGUAC |
| | | CCCACCAUCUACCACCUGAGAAAGAAACUGGUGGACAGCACCG |
| | | ACAAGGCCGACCUGCGGCUGAUCUAUCUGGCCCUGGCCCACAU |
| | | GAUCAAGUUCCGGGGCCACUUCCUGAUCGAGGGCGACCUGAAC |
| | | CCCGACAACAGCGACGUGGACAAGCUGUUCAUCCAGCUGGUGC |
| | | AGACCUACAACCAGCUGUUCGAGGAAAACCCCAUCAACGCCAG |
| | | CGGCGUGGACGCCAAGGCCAUCCUGUCUGCCAGACUGAGCAAG |
| | | AGCAGACGGCUGGAAAAUCUGAUCGCCCAGCUGCCCGGCGAGA |
| | | AGAAGAAUGGCCUGUUCGGAAACCUGAUUGCCCUGAGCCUGGG |
| | | CCUGACCCCCAACUUCAAGAGCAACUUCGACCUGGCCGAGGAU |
| | | GCCAAACUGCAGCUGAGCAAGGACACCUACGACGACGACCUGG |
| | | ACAACCUGCUGGCCCAGAUCGGCGACCAGUACGCCGACCUGUU |
| | | UCUGGCCGCCAAGAACCUGUCCGACGCCAUCCUGCUGAGCGAC |
| | | AUCCUGAGAGUGAACACCGAGAUCACCAAGGCCCCCCUGAGCG |
| | | CCUCUAUGAUCAAGAGAUACGACGAGCACCACCAGGACCUGAC |
| | | CCUGCUGAAAGCUCUCGUGCGGCAGCAGCUGCCUGAGAAGUAC |
| | | AAAGAGAUUUUCUUCGACCAGAGCAAGAACGGCUACGCCGGCU |
| | | ACAUUGACGGCGGAGCCAGCCAGGAAGAGUUCUACAAGUUCAU |
| | | CAAGCCCAUCCUGGAAAAGAUGGACGGCACCGAGGAACUGCUC |
| | | GUGAAGCUGAACAGAGAGGACCUGCUGCGGAAGCAGCGGACCU |
| | | UCGACAACGGCAGCAUCCCCCACCAGAUCCACCUGGGAGAGCU |
| | | GCACGCCAUUCUGCGGCGGCAGGAAGAUUUUUACCCAUUCCUG |
| | | AAGGACAACCGGGAAAAGAUCGAGAAGAUCCUGACCUUCCGCA |
| | | UCCCCUACUACGUGGGCCCUCUGGCCAGGGGAAACAGCAGAUU |
| | | CGCCUGGAUGACCAGAAAGAGCGAGGAAACCAUCACCCCCUGG |
| | | AACUUCGAGGAAGUGGUGGACAAGGGCGCUUCCGCCCAGAGCU |
| | | UCAUCGAGCGGAUGACCAACUUCGAUAAGAACCUGCCCAACGA |
| | | GAAGGUGCUGCCCAAGCACAGCCUGCUGUACGAGUACUUCACC |
| | | GUGUAUAACGAGCUGACCAAAGUGAAAUACGUGACCGAGGGA |
| | | AUGAGAAAGCCCGCCUUCCUGAGCGGCGAGCAGAAAAAGGCCA |
| | | UCGUGGACCUGCUGUUCAAGACCAACCGGAAAGUGACCGUGAA |
| | | GCAGCUGAAAGAGGACUACUUCAAGAAAAUCGAGUGCUUCGAC |
| | | UCCGUGGAAAUCUCCGGCGUGGAAGAUCGGUUCAACGCCUCCC |
| | | UGGGCACAUACCACGAUCUGCUGAAAAUUAUCAAGGACAAGGA |
| | | CUUCCUGGACAAUGAGGAAAACGAGGACAUUCUGGAAGAUAU |
| | | CGUGCUGACCCUGACACUGUUUGAGGACAGAGAGAUGAUCGAG |
| | | GAACGGCUGAAAACCUAUGCCCACCUGUUCGACGACAAAGUGA |
| | | UGAAGCAGCUGAAGCGGCGGAGAUACACCGGCUGGGGCAGGCU |
| | | GAGCCGGAAGCUGAUCAACGGCAUCCGGGACAAGCAGUCCGGC |
| | | AAGACAAUCCUGGAUUUCCUGAAGUCCGACGGCUUCGCCAACA |
| | | GAAACUUCAUGCAGCUGAUCCACGACGACAGCCUGACCUUUAA |
| | | AGAGGACAUCCAGAAAGCCCAGGUGUCCGGCCAGGGCGAUAGC |
| | | CUGCACGAGCACAUUGCCAAUCUGGCCGGCAGCCCCGCCAUUA |
| | | AGAAGGGCAUCCUGCAGACAGUGAAGGUGGUGGACGAGCUCG |
| | | UGAAAGUGAUGGGCCGGCACAAGCCCGAGAACAUCGUGAUCGA |
| | | AAUGGCCAGAGAGAACCAGACCACCCAGAAGGGACAGAAGAAC |
| | | AGCCGCGAGAGAAUGAAGCGGAUCGAAGAGGGCAUCAAAGAG |
| | | CUGGGCAGCCAGAUCCUGAAAGAACACCCCGUGGAAAACACCC |
| | | AGCUGCAGAACGAGAAGCUGUACCUGUACUACCUGCAGAAUGG |
| | | GCGGGAUAUGUACGUGGACCAGGAACUGGACAUCAACCGGCUG |
| | | UCCGACUACGAUGUGGACCAUAUCGUGCCUCAGAGCUUUCUGA |
| | | AGGACGACUCCAUCGACAACAAGGUGCUGACCAGAAGCGACAA |
| | | GAAUCGGGGCAAGAGCGACAACGUGCCCUCCGAAGAGGUCGUG |
| | | AAGAAGAUGAAGAACUACUGGCGGCAGCUGCUGAACGCCAAGC |
| | | UGAUUACCCAGAGAAAGUUCGACAAUCUGACCAAGGCCGAGAG |
| | | AGGCGGCCUGAGCGAACUGGAUAAGGCCGGCUUCAUCAAGAGA |
| | | CAGCUGGUGGAAACCCGGCAGAUCACAAAGCACGUGGCACAGA |
| | | UCCUGGACUCCCGGAUGAACACUAAGUACGACGAGAAUGACAA |
| | | GCUGAUCCGGGAAGUGAAAGUGAUCACCCUGAAGUCCAAGCUG |
| | | GUGUCCGAUUUCCGGAAGGAUUUCCAGUUUUACAAAGUGCGCG |
| | | AGAUCAACAACUACCACCACGCCCACGACGCCUACCUGAACGC |
| | | CGUCGUGGGAACCGCCCUGAUCAAAAAGUACCCUAAGCUGGAA |
| | | AGCGAGUUCGUGUACGGCGACUACAAGGUGUACGACGUGCGGA |
| | | AGAUGAUCGCCAAGAGCGAGCAGGAAAUCGGCAAGGCUACCGC |
| | | CAAGUACUUCUUCUACAGCAACAUCAUGAACUUUUUCAAGACC |
| | | GAGAUUACCCUGGCCAACGGCGAGAUCCGGAAGCGGCCUCUGA |
| | | UCGAGACAAACGGCGAAACCGGGGAGAUCGUGUGGGAUAAGG |
| | | GCCGGGAUUUUGCCACCGUGCGGAAAGUGCUGAGCAUGCCCCA |
| | | AGUGAAUAUCGUGAAAAAGACCGAGGUGCAGACAGGCGGCUU |
| | | CAGCAAAGAGUCUAUCCUGCCCAAGAGGAACAGCGAUAAGCUG |
| | | AUCGCCAGAAAGAAGGACUGGGACCCUAAGAAGUACGGCGGCU |
| | | UCGACAGCCCCACCGUGGCCUAUUCUGUGCUGGUGGUGGCCAA |
| | | AGUGGAAAAGGGCAAGUCCAAGAAACUGAAGAGUGUGAAAGA |
| | | GCUGCUGGGGAUCACCAUCAUGGAAAGAAGCAGCUUCGAGAAG |
| | | AAUCCCAUCGACUUUCUGGAAGCCAAGGGCUACAAAGAAGUGA |
| | | AAAAGGACCUGAUCAUCAAGCUGCCUAAGUACUCCCUGUUCGA |
| | | GCUGGAAAACGGCCGGAAGAGAAUGCUGGCCUCUGCCGGCGAA |
| | | CUGCAGAAGGGAAACGAACUGGCCCUGCCCUCCAAAUAUGUGA |
| | | ACUUCCUGUACCUGGCCAGCCACUAUGAGAAGCUGAAGGGCUC |
| | | CCCCGAGGAUAAUGAGCAGAAACAGCUGUUUGUGGAACAGCAC |
| | | AAGCACUACCUGGACGAGAUCAUCGAGCAGAUCAGCGAGUUCU |
| | | CCAAGAGAGUGAUCCUGGCCGACGCUAAUCUGGACAAAGUGCU |
| | | GUCCGCCUACAACAAGCACCGGGAUAAGCCCAUCAGAGAGCAG |
| | | GCCGAGAAUAUCAUCCACCUGUUUACCCUGACCAAUCUGGGAG |
| | | CCCCUGCCGCCUUCAAGUACUUUGACACCACCAUCGACCGGAA |
| | | GAGGUACACCAGCACCAAAGAGGUGCUGGACGCCACCCUGAUC |
| | | CACCAGAGCAUCACCGGCCUGUACGAGACACGGAUCGACCUGU |
| | | CUCAGCUGGGAGGUGACUCUGGAGGAUCUAGCGGAGGAUCCUC |
| | | UGGCAGCGAGACACCAGGAACAAGCGAGUCAGCAACACCAGAG |
| | | AGCAGUGGCGGCAGCAGCGGCGGCAGCAGCACCCUAAAUAUAG |
| | | AAGAUGAGUAUCGGCUACAUGAGACCUCAAAAGAGCCAGAUG |
| | | UUUCUCUAGGGUCCACAUGGCUGUCUGAUUUUCCUCAGGCCUG |
| | | GGCGGAAACCGGGGGCAUGGGACUGGCAGUUCGCCAAGCUCCU |
| | | CUGAUCAUACCUCUGAAAGCAACCUCUACCCCCGUGUCCAUAA |
| | | AACAAUACCCCAUGUCACAAGAAGCCAGACUGGGGAUCAAGCC |
| | | CCACAUACAGAGACUGUUGGACCAGGGAAUACUGGUACCCUGC |
| | | CAGUCCCCCUGGAACACGCCCCUGCUACCCGUUAAGAAACCAG |
| | | GGACUAAUGAUUAUAGGCCUGUCCAGGAUCUGAGAGAAGUCA |
| | | ACAAGCGGGUGGAGGACAUCCACCCCACCGUGCCCAACCCUUA |
| | | CAACCUCUUGAGCGGGCUCCCACCGUCCCACCAGUGGUACACU |
| | | GUGCUUGAUUUAAAGGAUGCCUUUUUCUGCCUGAGACUCCACC |
| | | CCACCAGUCAGCCUCUCUUCGCCUUUGAGUGGAGAGAUCCAGA |
| | | GAUGGGAAUCUCAGGACAAUUGACCUGGACCAGACUCCCACAG |
| | | GGUUUCAAAAACAGUCCCACCCUGUUUAAUGAGGCACUGCACA |
| | | GAGACCUAGCAGACUUCCGGAUCCAGCACCCAGACUUGAUCCU |
| | | GCUACAGUACGUGGAUGACUUACUGCUGGCCGCCACUUCUGAG |
| | | CUAGACUGCCAACAAGGUACUCGGGCCCUGUUACAAACCCUAG |
| | | GGAACCUCGGGUAUCGGGCCUCGGCCAAGAAAGCCCAAAUUUG |
| | | CCAGAAACAGGUCAAGUAUCUGGGGUAUCUUCUAAAAGAGGG |
| | | UCAGAGAUGGCUGACUGAGGCCAGAAAAGAGACUGUGAUGGG |
| | | GCAGCCUACUCCGAAGACCCCUCGACAACUAAGGGAGUUCCUA |
| | | GGGAAGGCAGGCUUCUGUCGCCUCUUCAUCCCUGGGUUUGCAG |
| | | AAAUGGCAGCCCCCCUGUACCCUCUCACCAAACCGGGGACUCU |
| | | GUUUAAUUGGGGCCCAGACCAACAAAAGGCCUAUCAAGAAAUC |
| | | AAGCAAGCCCUUCUAACUGCCCCAGCCCUGGGGUUGCCAGAUU |
| | | UGACUAAGCCCUUUGAACUCUUUGUCGACGAGAAGCAGGGCUA |
| | | CGCCAAAGGUGUCCUAACGCAAAAACUGGGACCUUGGCGUCGG |
| | | CCGGUGGCCUACCUGUCCAAAAAGCUAGACCCAGUAGCAGCUG |
| | | GGUGGCCCCCUUGCCUACGGAUGGUAGCAGCCAUUGCCGUACU |
| | | GACAAAGGAUGCAGGCAAGCUAACCAUGGGACAGCCACUAGUC |
| | | AUUCUGGCCCCCCAUGCAGUAGAGGCACUAGUCAAACAACCCC |
| | | CCGACCGCUGGCUUUCCAACGCCCGGAUGACUCACUAUCAGGC |
| | | CUUGCUUUUGGACACGGACCGGGUCCAGUUCGGACCGGUGGUA |
| | | GCCCUGAACCCGGCUACGCUGCUCCCACUGCCUGAGGAAGGGC |
| | | UGCAACACAACUGCCUUGAUAUCCUGGCCGAAGCCCACGGAAC |
| | | CCGACCCGACCUAACGGACCAGCCGCUCCCAGACGCCGACCACA |
| | | CCUGGUACACGGAUGGAAGCAGUCUCUUACAAGAGGGACAGCG |
| | | UAAGGCGGGAGCUGCGGUGACCACCGAGACCGAGGUAAUCUGG |
| | | GCUAAAGCCCUGCCAGCCGGGACAUCCGCUCAGCGGGCUGAAC |
| | | UGAUAGCACUCACCCAGGCCCUAAAGAUGGCAGAAGGUAAGAA |
| | | GCUAAAUGUUUAUACUGAUAGCCGUUAUGCUUUUGCUACUGCC |
| | | CAUAUCCAUGGAGAAAUAUACAGAAGGCGUGGGUGGCUCACA |
| | | UCAGAAGGCAAAGAGAUCAAAAAUAAAGACGAGAUCUUGGCC |
| | | CUACUAAAAGCCCUCUUUCUGCCCAAAAGACUUAGCAUAAUCC |
| | | AUUGUCCAGGACAUCAAAAGGGACACAGCGCCGAGGCUAGAGG |
| | | CAACCGGAUGGCUGACCAAGCGGCCCGAAAGGCAGCCAUCACA |
| | | GAGACUCCAGACACCUCUACCCUCCUCAUAGAAAAUUCAUCAC |
| | | CCUCUGGCGGCUCAAAAAGAACCGCCGACGGCAGCGAAUUCGA |
| | | GAAAAGGACGGCGGAUGGUAGCGAAUUCGAGAGCCCUAAAAA |
| | | GAAGGCCAAGGUAGAGUAA |
| guide RNA | 30434 | mA*mC*mA*CAAAUACCAGUCCAGCGGUUUUAGAmGmCmUmAm |
| | | GmAmAmAmUmAmGmCAAGUUAAAAUAAGGCUAGUCCGUUAUC |
| | | AmAmCmUmUmGmAmAmAmAmAmGmUmGmGmCmAmCmCmGmA |
| | | mGmUmCmGmGmUmGmCmU*mU*mU*mU |
| | | m=2′OMethyl, *= phosphorothioate linkage |

Lipid nanoparticle (LNP) components (ionizable lipid, helper lipid, sterol, PEG) were dissolved in 100% ethanol with the lipid component molar ratios of 47:8:43.5:1.5, respectively. RNA (guide and mRNA) was combined in a 1:1 weight ratio and diluted to a concentration of 0.05-0.2 mg/mL in sodium acetate buffer, pH 5. RNA was formulated into distinct LNPs with a lipid amine to total RNA phosphate (N:P) molar ratio of 4.0. The LNPs were formed by microfluidic or turbulent mixing of the lipid and RNA solutions. A 3:1 ratio of aqueous to organic solvent was maintained during mixing using differential flow rates. After mixing, the LNPs were diluted, collected and buffer exchanged into 50 mM Tris, 9% sucrose buffer using tangential flow filtration. Formulations were concentrated to 1.0 mg/mL or higher then filtered through 0.2 μm sterile filter. The final LNP were stored at −80° C. until further use.

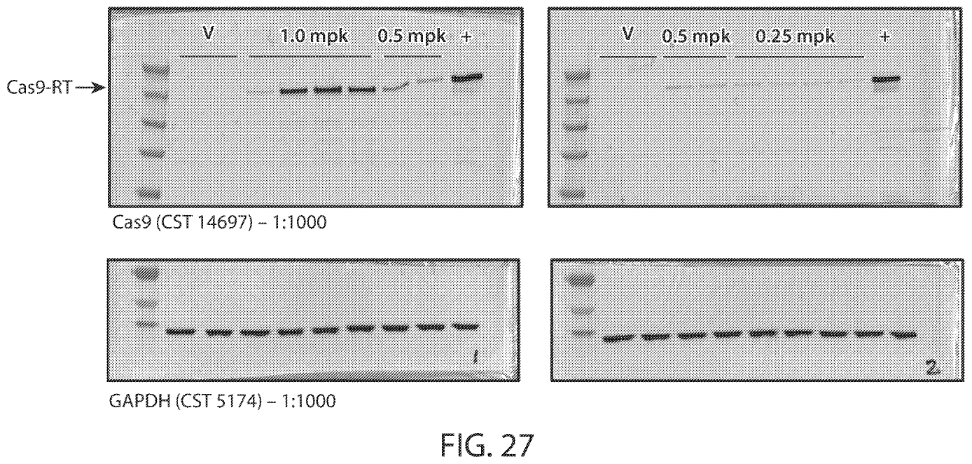

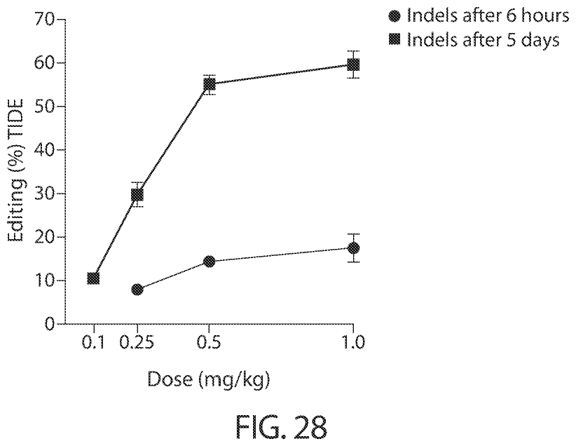

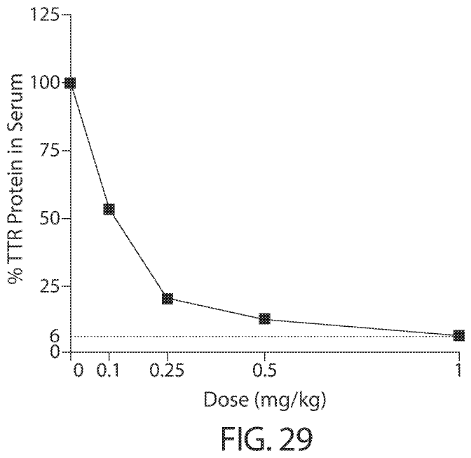

The LNP formulations were delivered intravenously by bolus tail vein injection to C57Blk/6 mice that were approximately 8 weeks old at concentrations ranging from 1-0.1 mg/kg. The expression of the Cas9-RT was measured by 6 hours after injection by euthanizing animals and collecting livers during necropsy. Animals were euthanized at 5 days after injection where liver was collected upon necropsy to which the activity of gene editing of the TTR locus was assessed. Expression of the Cas9-RT gene editing polypeptide in liver was measured by Western blot where Cas9 was detected by a mouse monoclonal antibody (7A9-3A3, Cell Signaling Technology) and GAPDH (Cell Signaling Technology) was used as a loading control. (FIG. 27). Editing of the TTR locus was quantified by Sanger sequencing followed by TIDE analysis of an amplicon of the TTR locus near the binding site of the protospacer. Editing of the TTR locus was observed, as shown in FIG. 28. TTR protein levels in serum were quantified by an ELISA using a standard curve (Aviva Biosciences). TTR protein levels in serum declined in treated animals, as shown in FIG. 29. These experiments demonstrate that the Cas9-RT polypeptide can be expressed in vivo, and can edit the TTR locus, resulting in a decrease in TTR protein levels in serum.

### Example 19. Gene Editing at the TTR Locus in an In Vivo Cynomolgus Macaque Model

This Example demonstrates successful delivery of an mRNA and guide using Cas9-mediated gene editing using the protospacer sequence ACACAAAUACCAGUCCAGCG (SEQ ID NO: 37641) that targets the TTR locus using a heterologous gene modifying polypeptide and RNA in a cynomolgus model.

RNAs were prepared as follows. An mRNA encoding a heterologous gene modifying polypeptide having the sequence shown in Table 6A1 below was produced by in vitro transcription and the purified mRNA was dissolved in 1 mM sodium citrate, pH 6, to a final concentration of RNA of 1-2 mg/mL. Similarly, a guide RNA having a sequence shown in Table 6A1 below was produced by chemical synthesis and dissolved in water or aqueous buffer, to a final concentration of RNA of 1-2 mg/mL.

TABLE 6A1
Sequences of Example 19

| Name | SEQ ID NO | Nucleic acid sequence |
|---|---|---|
| Cas9-RT gene | 30435 | AUGCCUGCGGCUAAGCGGGUAAAAUUGGAUGGUGGGGACAAGAAGUACAGCAUC |
| modifying | | GGCCUGGACAUCGGCACCAACUCUGUGGGCUGGGCCGUGAUCACCGACGAGUACA |
| polypeptide | | AGGUGCCCAGCAAGAAAUUCAAGGUGCUGGGCAACACCGACCGGCACAGCAUCAA |
| | | GAAGAACCUGAUCGGAGCCCUGCUGUUCGACAGCGGCGAAACAGCCGAGGCCACC |
| | | CGGCUGAAGAGAACCGCCAGAAGAAGAUACACCAGACGGAAGAACCGGAUCUGC |
| | | UAUCUGCAAGAGAUCUUCAGCAACGAGAUGGCCAAGGUGGACGACAGCUUCUUC |
| | | CACAGACUGGAAGAGUCCUUCCUGGUGGAAGAGGAUAAGAAGCACGAGCGGCAC |
| | | CCCAUCUUCGGCAACAUCGUGGACGAGGUGGCCUACCACGAGAAGUACCCCACCA |
| | | UCUACCACCUGAGAAAGAAACUGGUGGACAGCACCGACAAGGCCGACCUGCGGCU |
| | | GAUCUAUCUGGCCCUGGCCCACAUGAUCAAGUUCCGGGGCCACUUCCUGAUCGAG |
| | | GGCGACCUGAACCCCGACAACAGCGACGUGGACAAGCUGUUCAUCCAGCUGGUGC |
| | | AGACCUACAACCAGCUGUUCGAGGAAAACCCCAUCAACGCCAGCGGCGUGGACGC |
| | | CAAGGCCAUCCUGUCUGCCAGACUGAGCAAGAGCAGACGGCUGGAAAAUCUGAUC |
| | | GCCCAGCUGCCCGGCGAGAAGAAGAAUGGCCUGUUCGGAAACCUGAUUGCCCUGA |
| | | GCCUGGGCCUGACCCCCAACUUCAAGAGCAACUUCGACCUGGCCGAGGAUGCCAA |
| | | ACUGCAGCUGAGCAAGGACACCUACGACGACGACCUGGACAACCUGCUGGCCCAG |
| | | AUCGGCGACCAGUACGCCGACCUGUUUCUGGCCGCCAAGAACCUGUCCGACGCCA |
| | | UCCUGCUGAGCGACAUCCUGAGAGUGAACACCGAGAUCACCAAGGCCCCCCUGAG |
| | | CGCCUCUAUGAUCAAGAGAUACGACGAGCACCACCAGGACCUGACCCUGCUGAAA |
| | | GCUCUCGUGCGGCAGCAGCUGCCUGAGAAGUACAAAGAGAUUUUCUUCGACCAG |
| | | AGCAAGAACGGCUACGCCGGCUACAUUGACGGCGGAGCCAGCCAGGAAGAGUUCU |
| | | ACAAGUUCAUCAAGCCCAUCCUGGAAAAGAUGGACGGCACCGAGGAACUGCUCGU |
| | | GAAGCUGAACAGAGAGGACCUGCUGCGGAAGCAGCGGACCUUCGACAACGGCAGC |
| | | AUCCCCCACCAGAUCCACCUGGGAGAGCUGCACGCCAUUCUGCGGCGGCAGGAAG |
| | | AUUUUUACCCAUUCCUGAAGGACAACCGGGAAAAGAUCGAGAAGAUCCUGACCU |
| | | UCCGCAUCCCCUACUACGUGGGCCCUCUGGCCAGGGGAAACAGCAGAUUCGCCUG |
| | | GAUGACCAGAAAGAGCGAGGAAACCAUCACCCCCUGGAACUUCGAGGAAGUGGU |
| | | GGACAAGGGCGCUUCCGCCCAGAGCUUCAUCGAGCGGAUGACCAACUUCGAUAAG |
| | | AACCUGCCCAACGAGAAGGUGCUGCCCAAGCACAGCCUGCUGUACGAGUACUUCA |
| | | CCGUGUAUAACGAGCUGACCAAAGUGAAAUACGUGACCGAGGGAAUGAGAAAGC |
| | | CCGCCUUCCUGAGCGGCGAGCAGAAAAAGGCCAUCGUGGACCUGCUGUUCAAGAC |
| | | CAACCGGAAAGUGACCGUGAAGCAGCUGAAAGAGGACUACUUCAAGAAAAUCGA |
| | | GUGCUUCGACUCCGUGGAAAUCUCCGGCGUGGAAGAUCGGUUCAACGCCUCCCUG |
| | | GGCACAUACCACGAUCUGCUGAAAAUUAUCAAGGACAAGGACUUCCUGGACAAU |
| | | GAGGAAAACGAGGACAUUCUGGAAGAUAUCGUGCUGACCCUGACACUGUUUGAG |
| | | GACAGAGAGAUGAUCGAGGAACGGCUGAAAACCUAUGCCCACCUGUUCGACGAC |
| | | AAAGUGAUGAAGCAGCUGAAGCGGCGGAGAUACACCGGCUGGGGCAGGCUGAGC |
| | | CGGAAGCUGAUCAACGGCAUCCGGGACAAGCAGUCCGGCAAGACAAUCCUGGAUU |
| | | UCCUGAAGUCCGACGGCUUCGCCAACAGAAACUUCAUGCAGCUGAUCCACGACGA |
| | | CAGCCUGACCUUUAAAGAGGACAUCCAGAAAGCCCAGGUGUCCGGCCAGGGCGAU |
| | | AGCCUGCACGAGCACAUUGCCAAUCUGGCCGGCAGCCCCGCCAUUAAGAAGGGCA |
| | | UCCUGCAGACAGUGAAGGUGGUGGACGAGCUCGUGAAAGUGAUGGGCCGGCACA |
| | | AGCCCGAGAACAUCGUGAUCGAAAUGGCCAGAGAGAACCAGACCACCCAGAAGGG |
| | | ACAGAAGAACAGCCGCGAGAGAAUGAAGCGGAUCGAAGAGGGCAUCAAAGAGCU |
| | | GGGCAGCCAGAUCCUGAAAGAACACCCCGUGGAAAACACCCAGCUGCAGAACGAG |
| | | AAGCUGUACCUGUACUACCUGCAGAAUGGGCGGGAUAUGUACGUGGACCAGGAA |
| | | CUGGACAUCAACCGGCUGUCCGACUACGAUGUGGACCAUAUCGUGCCUCAGAGCU |
| | | UUCUGAAGGACGACUCCAUCGACAACAAGGUGCUGACCAGAAGCGACAAGAAUC |
| | | GGGGCAAGAGCGACAACGUGCCCUCCGAAGAGGUCGUGAAGAAGAUGAAGAACU |
| | | ACUGGCGGCAGCUGCUGAACGCCAAGCUGAUUACCCAGAGAAAGUUCGACAAUCU |
| | | GACCAAGGCCGAGAGAGGCGGCCUGAGCGAACUGGAUAAGGCCGGCUUCAUCAA |
| | | GAGACAGCUGGUGGAAACCCGGCAGAUCACAAAGCACGUGGCACAGAUCCUGGAC |
| | | UCCCGGAUGAACACUAAGUACGACGAGAAUGACAAGCUGAUCCGGGAAGUGAAA |
| | | GUGAUCACCCUGAAGUCCAAGCUGGUGUCCGAUUUCCGGAAGGAUUUCCAGUUU |
| | | UACAAAGUGCGCGAGAUCAACAACUACCACCACGCCCACGACGCCUACCUGAACG |
| | | CCGUCGUGGGAACCGCCCUGAUCAAAAAGUACCCUAAGCUGGAAAGCGAGUUCGU |
| | | GUACGGCGACUACAAGGUGUACGACGUGCGGAAGAUGAUCGCCAAGAGCGAGCA |
| | | GGAAAUCGGCAAGGCUACCGCCAAGUACUUCUUCUACAGCAACAUCAUGAACUUU |
| | | UUCAAGACCGAGAUUACCCUGGCCAACGGCGAGAUCCGGAAGCGGCCUCUGAUCG |
| | | AGACAAACGGCGAAACCGGGGAGAUCGUGUGGGAUAAGGGCCGGGAUUUUGCCA |
| | | CCGUGCGGAAAGUGCUGAGCAUGCCCCAAGUGAAUAUCGUGAAAAAGACCGAGG |
| | | UGCAGACAGGCGGCUUCAGCAAAGAGUCUAUCCUGCCCAAGAGGAACAGCGAUA |
| | | AGCUGAUCGCCAGAAAGAAGGACUGGGACCCUAAGAAGUACGGCGGCUUCGACA |
| | | GCCCCACCGUGGCCUAUUCUGUGCUGGUGGUGGCCAAAGUGGAAAAGGGCAAGU |
| | | CCAAGAAACUGAAGAGUGUGAAAGAGCUGCUGGGGAUCACCAUCAUGGAAAGAA |
| | | GCAGCUUCGAGAAGAAUCCCAUCGACUUUCUGGAAGCCAAGGGCUACAAAGAAG |
| | | UGAAAAAGGACCUGAUCAUCAAGCUGCCUAAGUACUCCCUGUUCGAGCUGGAAA |
| | | ACGGCCGGAAGAGAAUGCUGGCCUCUGCCGGCGAACUGCAGAAGGGAAACGAAC |
| | | UGGCCCUGCCCUCCAAAUAUGUGAACUUCCUGUACCUGGCCAGCCACUAUGAGAA |
| | | GCUGAAGGGCUCCCCCGAGGAUAAUGAGCAGAAACAGCUGUUUGUGGAACAGCA |
| | | CAAGCACUACCUGGACGAGAUCAUCGAGCAGAUCAGCGAGUUCUCCAAGAGAGU |
| | | GAUCCUGGCCGACGCUAAUCUGGACAAAGUGCUGUCCGCCUACAACAAGCACCGG |
| | | GAUAAGCCCAUCAGAGAGCAGGCCGAGAAUAUCAUCCACCUGUUUACCCUGACCA |
| | | AUCUGGGAGCCCCUGCCGCCUUCAAGUACUUUGACACCACCAUCGACCGGAAGAG |
| | | GUACACCAGCACCAAAGAGGUGCUGGACGCCACCCUGAUCCACCAGAGCAUCACC |
| | | GGCCUGUACGAGACACGGAUCGACCUGUCUCAGCUGGGAGGUGACUCUGGAGGA |
| | | UCUAGCGGAGGAUCCUCUGGCAGCGAGACACCAGGAACAAGCGAGUCAGCAACAC |
| | | CAGAGAGCAGUGGCGGCAGCAGCGGCGGCAGCAGCACCCUAAAUAUAGAAGAUG |
| | | AGUAUCGGCUACAUGAGACCUCAAAAGAGCCAGAUGUUUCUCUAGGGUCCACAU |
| | | GGCUGUCUGAUUUUCCUCAGGCCUGGGCGGAAACCGGGGGCAUGGGACUGGCAG |
| | | UUCGCCAAGCUCCUCUGAUCAUACCUCUGAAAGCAACCUCUACCCCCGUGUCCAU |
| | | AAAACAAUACCCCAUGUCACAAGAAGCCAGACUGGGGAUCAAGCCCCACAUACAG |
| | | AGACUGUUGGACCAGGGAAUACUGGUACCCUGCCAGUCCCCCUGGAACACGCCCC |
| | | UGCUACCCGUUAAGAAACCAGGGACUAAUGAUUAUAGGCCUGUCCAGGAUCUGA |
| | | GAGAAGUCAACAAGCGGGUGGAGGACAUCCACCCCACCGUGCCCAACCCUUACAA |
| | | CCUCUUGAGCGGGCUCCCACCGUCCCACCAGUGGUACACUGUGCUUGAUUUAAAG |
| | | GAUGCCUUUUUCUGCCUGAGACUCCACCCCACCAGUCAGCCUCUCUUCGCCUUUG |
| | | AGUGGAGAGAUCCAGAGAUGGGAAUCUCAGGACAAUUGACCUGGACCAGACUCC |
| | | CACAGGGUUUCAAAAACAGUCCCACCCUGUUUAAUGAGGCACUGCACAGAGACCU |
| | | AGCAGACUUCCGGAUCCAGCACCCAGACUUGAUCCUGCUACAGUACGUGGAUGAC |
| | | UUACUGCUGGCCGCCACUUCUGAGCUAGACUGCCAACAAGGUACUCGGGCCCUGU |
| | | UACAAACCCUAGGGAACCUCGGGUAUCGGGCCUCGGCCAAGAAAGCCCAAAUUUG |
| | | CCAGAAACAGGUCAAGUAUCUGGGGUAUCUUCUAAAAGAGGGUCAGAGAUGGCU |
| | | GACUGAGGCCAGAAAAGAGACUGUGAUGGGGCAGCCUACUCCGAAGACCCCUCGA |
| | | CAACUAAGGGAGUUCCUAGGGAAGGCAGGCUUCUGUCGCCUCUUCAUCCCUGGGU |
| | | UUGCAGAAAUGGCAGCCCCCCUGUACCCUCUCACCAAACCGGGGACUCUGUUUAA |
| | | UUGGGGCCCAGACCAACAAAAGGCCUAUCAAGAAAUCAAGCAAGCCCUUCUAACU |
| | | GCCCCAGCCCUGGGGUUGCCAGAUUUGACUAAGCCCUUUGAACUCUUUGUCGACG |
| | | AGAAGCAGGGCUACGCCAAAGGUGUCCUAACGCAAAAACUGGGACCUUGGCGUC |
| | | GGCCGGUGGCCUACCUGUCCAAAAAGCUAGACCCAGUAGCAGCUGGGUGGCCCCC |
| | | UUGCCUACGGAUGGUAGCAGCCAUUGCCGUACUGACAAAGGAUGCAGGCAAGCU |
| | | AACCAUGGGACAGCCACUAGUCAUUCUGGCCCCCCAUGCAGUAGAGGCACUAGUC |
| | | AAACAACCCCCCGACCGCUGGCUUUCCAACGCCCGGAUGACUCACUAUCAGGCCU |
| | | UGCUUUUGGACACGGACCGGGUCCAGUUCGGACCGGUGGUAGCCCUGAACCCGGC |
| | | UACGCUGCUCCCACUGCCUGAGGAAGGGCUGCAACACAACUGCCUUGAUAUCCUG |
| | | GCCGAAGCCCACGGAACCCGACCCGACCUAACGGACCAGCCGCUCCCAGACGCCG |
| | | ACCACACCUGGUACACGGAUGGAAGCAGUCUCUUACAAGAGGGACAGCGUAAGG |
| | | CGGGAGCUGCGGUGACCACCGAGACCGAGGUAAUCUGGGCUAAAGCCCUGCCAGC |
| | | CGGGACAUCCGCUCAGCGGGCUGAACUGAUAGCACUCACCCAGGCCCUAAAGAUG |
| | | GCAGAAGGUAAGAAGCUAAAUGUUUAUACUGAUAGCCGUUAUGCUUUUGCUACU |
| | | GCCCAUAUCCAUGGAGAAAUAUACAGAAGGCGUGGGUGGCUCACAUCAGAAGGC |
| | | AAAGAGAUCAAAAAUAAAGACGAGAUCUUGGCCCUACUAAAAGCCCUCUUUCUG |
| | | CCCAAAAGACUUAGCAUAAUCCAUUGUCCAGGACAUCAAAAGGGACACAGCGCCG |
| | | AGGCUAGAGGCAACCGGAUGGCUGACCAAGCGGCCCGAAAGGCAGCCAUCACAGA |
| | | GACUCCAGACACCUCUACCCUCCUCAUAGAAAAUUCAUCACCCUCUGGCGGCUCA |
| | | AAAAGAACCGCCGACGGCAGCGAAUUCGAGAAAAGGACGGCGGAUGGUAGCGAA |
| | | UUCGAGAGCCCUAAAAAGAAGGCCAAGGUAGAGUAA |
| guide RNA | 30436 | mA*mC*mA*CAAAUACCAGUCCAGCGGUUUUAGAmGmCmUmAmGmAmAmAmUmA |
| | | mGmCAAGUUAAAAUAAGGCUAGUCCGUUAUCAmAmCmUmUmGmAmAmAmAmAm |
| | | GmUmGmGmCmAmCmCmGmAmGmUmCmGmGmUmGmCmU*mU*mU*mU |
| | | m=2′OMethyl, *= phosphorothioate linkage |

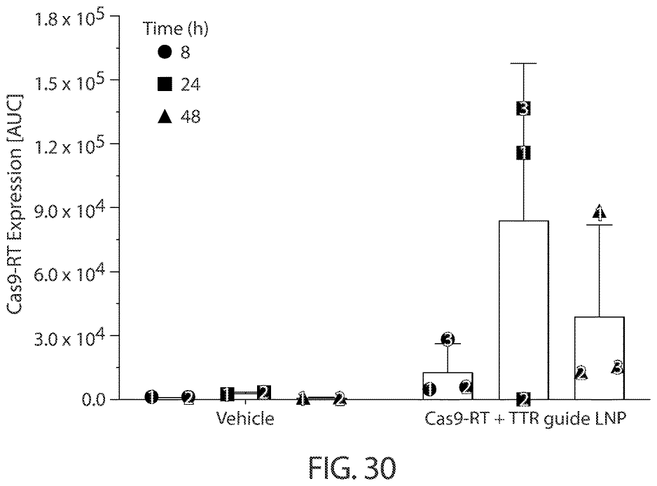

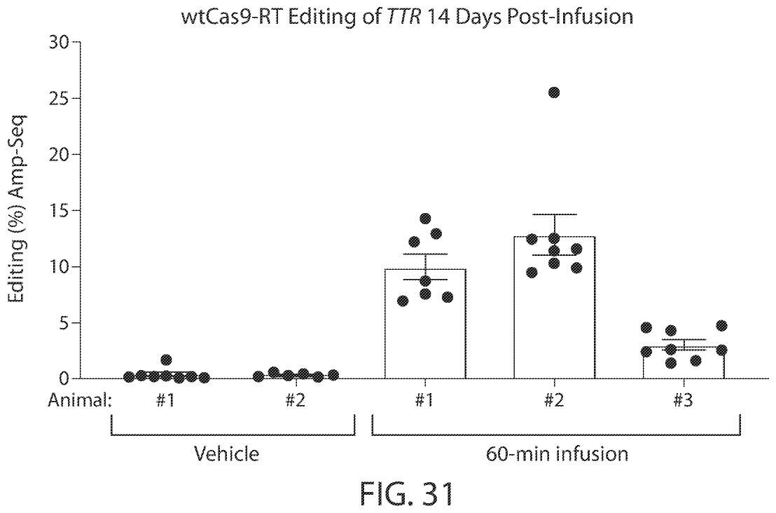

Lipid nanoparticle (LNP) components (ionizable lipid, helper lipid, sterol, PEG) were dissolved in 100% ethanol with the lipid component molar ratios of 47:8:43.5:1.5, respectively. RNA (guide and mRNA) was combined in a 1:1 weight ratio and diluted to a concentration of 0.05-0.2 mg/mL in sodium acetate buffer, pH 5. RNA was formulated into distinct LNPs with a lipid amine to total RNA phosphate (N:P) molar ratio of 4.0. The LNPs were formed by microfluidic or turbulent mixing of the lipid and RNA solutions. A 3:1 ratio of aqueous to organic solvent was maintained during mixing using differential flow rates. After mixing, the LNPs were diluted, collected and buffer exchanged into 50 mM Tris, 9% sucrose buffer using tangential flow filtration. Formulations were concentrated to 1.0 mg/mL or higher then filtered through 0.2 μm sterile filter. The final LNP were stored at −80° C. until further use. The LNP formulations were delivered intravenously by infusion over the course of 1 hour at 2 mg/kg where the volume of the infusion was 5 ml/kg. Cynomolgus macaques from mainland Asia were given dexamethasone 2 mg/kg bolus via intramuscular injection 1.5-2 h prior to intravenous infusion using a syringe pump. Animals were monitored after infusion and the expression of the Cas9-RT was measured by laparoscopic biopsies taken from the liver 8-12 h, 24 h, and 48 h after infusion. Animals were euthanized 14 days after infusion and liver was harvested by dividing the organ up into 8 different segments to which the activity of gene editing of the TTR locus was assessed. Expression of the Cas9-RT gene editing polypeptide in liver was quantified by capillary electrophoresis western blot using the ProteinSimple Jess system (bio-techne) where Cas9 was detected by a mouse monoclonal antibody (7A9-3A3, Cell Signaling Technology). Relative expression of the Cas9-RT gene editing polypeptide was measured by an area under curve analysis, as shown in FIG. 30. Editing of the TTR locus was quantified by amplicon-sequencing of the TTR locus near the binding site of the protospacer. Editing of the TTR locus was observed, as shown in FIG. 31. These experiments demonstrate that the Cas9-RT polypeptide can be expressed in vivo in a non-human primate model and can edit the TTR locus.

It should be understood that for all numerical bounds describing some parameter in this application, such as “about,” “at least,” “less than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera.

For all patents, applications, or other reference cited herein, such as non-patent literature and reference sequence information, it should be understood that they are incorporated by reference in their entirety for all purposes as well as for the proposition that is recited. Where any conflict exists between a document incorporated by reference and the present application, this application will control. All information associated with reference gene sequences disclosed in this application, such as GeneIDs or accession numbers (typically referencing NCBI accession numbers), including, for example, genomic loci, genomic sequences, functional annotations, allelic variants, and reference mRNA (including, e.g., exon boundaries or response elements) and protein sequences (such as conserved domain structures), as well as chemical references (e.g., PubChem compound, PubChem substance, or PubChem Bioassay entries, including the annotations therein, such as structures and assays, et cetera), are hereby incorporated by reference in their entirety.

Headings used in this application are for convenience only and do not affect the interpretation of this application.

LENGTHY TABLES
The patent application contains a lengthy table section. A copy of the table is available in electronic form from the USPTO web site (https://seqdata.uspto.gov/docdetail?docId=US20260224743A1). An electronic copy of the table will also be available from the USPTO upon request and payment of the fee set forth in 37 CFR 1.19(b)(3).

1. A template RNA comprising, from 5′ to 3′:
(i) a gRNA spacer that is complementary to a first portion of the human TRAC gene, wherein the gRNA spacer has a sequence comprising the nucleotides of a gRNA spacer sequence of gagaaucaaaaucggugaau (SEQ ID NO. 17513) or gcugguacacggcaggguca SEQ ID NO. 17514);
(ii) a gRNA scaffold that binds a heterologous gene modifying polypeptide (e.g., binds the Cas domain of the heterologous gene modifying polypeptide),
(iii) a heterologous object sequence comprising a mutation region to introduce a mutation into a second portion of the human TRAC gene (wherein optionally the heterologous object sequence comprises, from 5′ to 3′, a post-edit homology region, a mutation region, and a pre-edit homology region), and
(iv) a primer binding site (PBS) sequence comprising at least 5, 6, 7, or 8 bases with 100% identity to a third portion of the human TRAC gene.

2. The template RNA of claim 1, wherein the heterologous object sequence comprises the nucleotides of an RT template sequence from Table 1A, 2A, or 3A, a sequence having at least 5, 6, 7, 8, 9, 10, 11, 12, 13, 14, or 15 nucleotides starting with the 3′ end of the RT template sequence of Table 1A, 2A, or 3A, or a sequence having 1, 2, or 3 substitutions thereto.

3. The template RNA of claim 1, wherein the heterologous object sequence comprises the nucleotides of the RT template sequence of Table 1A, 2A, or 3A that corresponds to the gRNA spacer sequence.

4. A template RNA comprising, from 5′ to 3′:
(i) a gRNA spacer that is complementary to a first portion of the human TRAC gene,
(ii) a gRNA scaffold that binds a heterologous gene modifying polypeptide (e.g., binds the Cas domain of the heterologous gene modifying polypeptide),
(iii) a heterologous object sequence comprising a mutation region to introduce a mutation into a second portion of the human TRAC gene, wherein the heterologous object sequence comprises the nucleotides of an RT template sequence of Table 1A, 2A, or 3A, a sequence having at least 5, 6, 7, 8, 9, 10, 11, 12, 13, 14, or 15 nucleotides starting with the 3′ end of the RT template sequence of Table 1A, 2A, or 3A, or a sequence having 1, 2, or 3 substitutions thereto; and
(iv) a PBS sequence comprising at least 5, 6, 7, or 8 bases of 100% identity to a third portion of the human TRAC gene.

5. The template RNA of claim 4, wherein the gRNA spacer comprises the nucleotides of a gRNA spacer sequence of gagaaucaaaaucggugaau (SEQ ID NO. 17513) or gcugguacacggcaggguca SEQ ID NO. 17514).

6. The template RNA of claim 4, wherein the heterologous object sequence comprises the nucleotides of the gRNA spacer sequence of gagaaucaaaaucggugaau (SEQ ID NO. 17513) or gcugguacacggcaggguca SEQ ID NO. 17514) that corresponds to the RT template sequence.

7. The template RNA according to any one of claims 1-6, wherein the PBS sequence has a sequence comprising the nucleotides of the PBS sequence from the same row of Table 1A, 2A, or 3A as the RT template sequence, a sequence having at least 5, 6, 7, 8, 9, 10, 11, 12, 13, 14, 15, 16, or 17 nucleotides starting with the 5′ end of a PBS sequence from Table 1A, 2A, or 3A.

8. The template RNA according any one of claims 1-6, wherein the PBS sequence has a sequence comprising the nucleotides of a PBS sequence of Table 1A that corresponds to the RT template sequence, the gRNA spacer sequence, or both.

9. The template RNA according to any one of claims 1-8, wherein the gRNA scaffold comprises a sequence of a gRNA scaffold of any of Tables 1A, 2A, 3A, 12, 14B, or 14C, or a sequence having at least 70%, 75%, 80%, 85%, 90%, 95%, 96%, 97%, 98%, or 99% identity thereto.

10. The template RNA according to any one of claims 1-8, wherein the gRNA scaffold comprises a sequence of a gRNA scaffold of any of Tables 1A, 2A, 3A, 12, 14B, or 14C that corresponds to the RT template sequence, the gRNA spacer sequence, or both, or a sequence having at least 70%, 75%, 80%, 85%, 90%, 95%, 96%, 97%, 98%, or 99% identity thereto.

11. A template RNA comprising, from 5′ to 3′:
(i) a gRNA spacer that is complementary to a first portion of the human B2M gene, wherein the gRNA spacer has a sequence comprising the nucleotides of a gRNA spacer sequence of acUcacgcUggaUagccUcc (SEQ ID NO: 18195) or gagUagcgcgagcacagcUa (SEQ ID NO: 18196);
(ii) a gRNA scaffold that binds a heterologous gene modifying polypeptide (e.g., binds the Cas domain of the heterologous gene modifying polypeptide),
(iii) a heterologous object sequence comprising a mutation region to introduce a mutation into a second portion of the human B2M gene (wherein optionally the heterologous object sequence comprises, from 5′ to 3′, a post-edit homology region, a mutation region, and a pre-edit homology region), and
(iv) a primer binding site (PBS) sequence comprising at least 5, 6, 7, or 8 bases with 100% identity to a third portion of the human B2M gene.

12. The template RNA of claim 11, wherein the heterologous object sequence comprises the nucleotides of an RT template sequence from Table 1B, 2B, or 3B, a sequence having at least 4, 6, 7, 8, 9, 10, 11, 12, 13, 14, or 15 nucleotides starting with the 3′ end of the RT template sequence of Table 1B, 2B, or 3B, or a sequence having 1, 2, or 3 substitutions thereto.

13. The template RNA of claim 11, wherein the heterologous object sequence comprises the nucleotides of the RT template sequence of Table 1B, 2B, or 3B that corresponds to the gRNA spacer sequence.

14. A template RNA comprising, from 5′ to 3′:
(i) a gRNA spacer that is complementary to a first portion of the human B2M gene,
(ii) a gRNA scaffold that binds a heterologous gene modifying polypeptide (e.g., binds the Cas domain of the heterologous gene modifying polypeptide),
(iii) a heterologous object sequence comprising a mutation region to introduce a mutation into a second portion of the human B2M gene, wherein the heterologous object sequence comprises the nucleotides of an RT template sequence of Table 1B, 2B, or 3B, a sequence having at least 5, 6, 7, 8, 9, 10, or 11 nucleotides starting with the 3′ end of the RT template sequence of Table 1B, 2B, or 3B, or a sequence having 1, 2, or 3 substitutions thereto; and
(iv) a PBS sequence comprising at least 5, 6, 7, or 8 bases of 100% identity to a third portion of the human B2M gene.

15. The template RNA of claim 14, wherein the gRNA spacer comprises the nucleotides of a gRNA spacer sequence of acUcacgcUggaUagccUcc (SEQ ID NO: 18195) or gagUagcgcgagcacagcUa (SEQ ID NO: 18196).

16. The template RNA of claim 14, wherein the heterologous object sequence comprises the nucleotides of the gRNA spacer sequence of acUcacgcUggaUagccUcc (SEQ ID NO: 18195) or gagUagcgcgagcacagcUa (SEQ ID NO: 18196) that corresponds to the RT template sequence.

17. The template RNA according to any one of claims 11-16, wherein the PBS sequence has a sequence comprising the nucleotides of the PBS sequence from the same row of Table 1B, 2B, or 3B as the RT template sequence, a sequence having at least 5, 6, 7, 8, 9, 10, 11, 12, 13, 14, 15, 16, or 17 nucleotides starting with the 5′ end of a PBS sequence from Table 1B, 2B, or 3B.

18. The template RNA according any one of claims 11-16, wherein the PBS sequence has a sequence comprising the nucleotides of a PBS sequence of Table 1B that corresponds to the RT template sequence, the gRNA spacer sequence, or both.

19. The template RNA according to any one of claims 11-18, wherein the gRNA scaffold comprises a sequence of a gRNA scaffold of any of Tables 1B, 2B, 3B, or 12, or a sequence having at least 70%, 75%, 80%, 85%, 90%, 95%, 96%, 97%, 98%, or 99% identity thereto.

20. The template RNA according to any one of claims 11-18, wherein the gRNA scaffold comprises a sequence of a gRNA scaffold of any of Tables 1B, 2B, 3B, or 12 that corresponds to the RT template sequence, the gRNA spacer sequence, or both, or a sequence having at least 70%, 75%, 80%, 85%, 90%, 95%, 96%, 97%, 98%, or 99% identity thereto.

21. The template RNA of any one of claims 1-20, wherein the pre-edit sequence comprises between about 1 nucleotide to about 35 nucleotides (e.g., comprises about 1-5, 5-10, 10-15, 15-20, 20-25, 25-30, or 30-35 nucleotides) in length.

22. The template RNA of any one of claims 1-21, wherein the mutation region comprises at least 4 or 5 nucleotides (e.g., at least 4 nucleotides, at least 5 nucleotides, at least 7 nucleotides, or more).

23. The template RNA of any one of claims 1-21, wherein the mutation region is at least 7 nucleotides in length.

24. The template RNA of any one of claims 1-21, wherein the mutation region is at least 15 nucleotides in length.

25. The template RNA of any one of claim 1-21, 23, or 24, wherein the mutation region is up to 32 (e.g., up to 5, 10, 15, 20, 25, 30, or 32) nucleotides in length and comprises at least 4 or 5 sequence differences relative to a second portion of the human TRAC gene or the human B2M gene.

26. The template RNA of any one of claim 1-21 or 23-25, wherein the mutation region comprises a first region (e.g., a first nucleotide) designed to install a mutation in the TRAC gene or the B2M gene and a second region (e.g., a second nucleotide) designed to inactivate a PAM sequence.

27. The template RNA of any one of claims 1-26, wherein the mutation region comprises less than 80%, 70%, 60%, 50%, 40%, or 30% identity to the corresponding portion of the human TRAC gene or the B2M gene.

28. The template RNA according to any of claims 1-27, wherein the template RNA comprises one or more chemical modifications.

29. A heterologous gene modifying system comprising:
a template RNA of any of claims 1-28, and
a heterologous gene modifying polypeptide, or a nucleic acid (e.g., RNA) encoding the heterologous gene modifying polypeptide.

30. A heterologous gene modifying system comprising:
a template RNA of any of claim 1-10 or 21-28,
a template RNA of any of claims 11-28,
a heterologous gene modifying polypeptide, or a nucleic acid (e.g., RNA) encoding the heterologous gene modifying polypeptide.

31. The heterologous gene modifying system of claim 30, wherein the heterologous gene modifying polypeptide comprises:
a reverse transcriptase (RT) domain (e.g., an RT domain from a retrovirus, or a polypeptide domain having at least 75%, 80%, 85%, 90%, 95%, 96%, 97%, 98%, or 99% amino acids sequence identity thereto); and
a Cas domain that binds to the target DNA molecule and is heterologous to the RT domain (e.g., a Cas9 domain); and
optionally, a linker disposed between the RT domain and the Cas domain.

32. The heterologous gene modifying system of claim 31, wherein the RT domain comprises:
(a) an RT domain of Table 6; or
(b) an RT domain from a murine leukemia virus (MMLV), a porcine endogenous retrovirus (PERV); Avian reticuloendotheliosis virus (AVIRE), a feline leukemia virus (FLV), simian foamy virus (SFV) (e.g., SFV3L), bovine leukemia virus (BLV), Mason-Pfizer monkey virus (MPMV), human foamy virus (HFV), or bovine foamy/syncytial virus (BFV/BSV).

33. The heterologous gene modifying system of claim 31 or 32, wherein the Cas domain comprises a Cas domain of Table 7 or Table 8.

34. The heterologous gene modifying system of claim 31 or 32, wherein the Cas domain:
(a) is a Cas9 domain;
(b) is a SpCas9 domain, a BlatCas9 domain, a Nme2Cas9 domain, a PnpCas9 domain, a SauCas9 domain, a SauCas9-KKH domain, a SauriCas9 domain, a SauriCas9-KKH domain, a ScaCas9-Sc++ domain, a SpyCas9 domain, a SpyCas9-NG domain, a SpyCas9-SpRY domain, or a StlCas9 domain; and/or
(c) is a Cas9 domain comprising an N670A mutation, an N611A mutation, an N605A mutation, an N580A mutation, an N588A mutation, an N872A mutation, an N863 mutation, an N622A mutation, or an H840A mutation.

35. The heterologous gene modifying system of claim 34, wherein the Cas9 domain binds a PAM sequence listed in Table 7 or Table 12.

36. The heterologous gene modifying system of claim 35, wherein a second portion of the human TRAC gene and/or the human B2M gene overlaps with a PAM recognized by the Cas domain, e.g., wherein the second portion of the human TRAC gene and/or the B2M gene is within the PAM or wherein the PAM is within the second portion of the human TRAC gene and/or the B2M gene).

37. The heterologous gene modifying system of any one of claims 29-36, wherein:
(a) the template RNA comprises a sequence of a template RNA sequence of Table 1A, 1B, 2A, 2B, 3A, or 3B;
(b) the Cas domain comprises a Cas domain of Table 7 or Table 8;
(c) the linker comprises a linker sequence of Table 10 (e.g., of any of SEQ ID NOs: 5217, 5106, 5190, and 5218); and
(d) the heterologous gene modifying polypeptide comprises one or two NLS sequences from Table 11 (e.g., of any of SEQ ID NOs: 5245, 5290, 5323, 5330, 5349, 5350, 5351, and 4001).

38. The heterologous gene modifying system of any one of claims 29-37, wherein the gRNA spacer targets a region within exon 1 of the TRAC gene.

39. The heterologous gene modifying system of any one of claims 29-38, wherein gRNA spacer comprises a sequence of gagaaucaaaaucggugaau (SEQ ID NO. 17513).

40. The heterologous gene modifying system of any one of claims 29-38, wherein the gRNA spacer comprises a sequence of gcugguacacggcaggguca (SEQ ID NO. 17514).

41. The heterologous gene modifying system of any one of claims 29-37, wherein the gRNA spacer targets a region within exon 1 of the B2M gene.

42. The heterologous gene modifying system of any one of claim 29-37 or 41, wherein the gRNA spacer comprises a sequence of acUcacgcUggaUagccUcc (SEQ ID NO: 18195).

43. The heterologous gene modifying system of any one of claim 29-37 or 41, wherein the gRNA spacer comprises a sequence of gagUagcgcgagcacagcUa (SEQ ID NO: 18196).

44. The heterologous gene modifying system of any of claims 29-43, which produces a first nick in a first strand of the human TRAC gene or the B2M gene.

45. The heterologous gene modifying system of claim 44, which further comprises a second strand-targeting gRNA spacer that directs a second nick to the second strand of the human TRAC gene or the B2M gene.

46. The template RNA or heterologous gene modifying system of any one of the preceding claims, wherein the heterologous object sequence comprises between about 8-30, 9-25, 10-20, 11-16, or 12-15 (e.g., about 11-16) nucleotides.

47. The template RNA or heterologous gene modifying system of any one of the preceding claims, wherein the mutation region comprises at least 4 or at least 5 nucleotide positions of sequence difference relative to the corresponding portion of the human TRAC gene or the B2M gene.

48. The template RNA or heterologous gene modifying system of any one of the preceding claims wherein the mutation region comprises at least 7 nucleotide positions of sequence difference relative to the corresponding portion of the human TRAC gene or the B2M gene.

49. The template RNA or heterologous gene modifying system, of any one of the preceding claims, wherein the post-edit homology region and/or pre-edit homology region comprises 100% identity to the TRAC gene or the B2M gene.

50. The template RNA or heterologous gene modifying system of any one of the preceding claims, wherein the PBS sequence comprises about 5-20, 8-16, 8-14, 8-13, 9-13, 9-12, or 10-12 (e.g., about 5-17) nucleotides.

51. The template RNA or heterologous gene modifying system of any one of the preceding claims, wherein the PBS sequence binds within 1, 2, 3, 4, 5, 6, 7, 8, 9, or 10 nucleotides of a nick site in the TRAC gene or the B2M gene.

52. The heterologous gene modifying system of any one of the preceding claims, wherein the domains of the heterologous gene modifying polypeptide are joined by a peptide linker.

53. The heterologous gene modifying system of claim 52, wherein the linker comprises a sequence of a linker of Table 10 (e.g., of any of SEQ ID NOs: 5217, 5106, 5190, and 5218).

54. The heterologous gene modifying system of any one of the preceding claims, wherein the heterologous gene modifying polypeptide further comprise one or more nuclear localization sequences (NLS).

55. The heterologous gene modifying system of claim 54, wherein the heterologous gene modifying polypeptide comprises a first NLS and a second NLS.

56. The heterologous gene modifying system of claim 54 or 55, wherein the NLS comprises a sequence of a NLS of Table 11 (e.g., of any of SEQ ID NOs: 5245, 5290, 5323, 5330, 5349, 5350, 5351, and 4001).

57. A template RNA comprising a sequence of a template RNA of Table 1A, 1B, 2A, 2B, 3A, or 3B, or a sequence having at least 70%, 75%, 80%, 85%, 90%, 95%, 96%, 97%, 98%, or 99% identity thereto.

58. A template RNA comprising a sequence of a template RNA of Table 1A, 1B, 2A, 2B, 3A, or 3B.

59. A DNA encoding the template RNA of any one of claim 1-28, 46-51, 57, or 58, or the heterologous gene modifying system of any one of claims 29-56.

60. A pharmaceutical composition, comprising the system of any one of claims 29-56, or one or more nucleic acids encoding the same, and a pharmaceutically acceptable excipient or carrier.

61. The pharmaceutical composition of claim 60, wherein the pharmaceutically acceptable excipient or carrier is selected from the group consisting of a plasmid vector, a viral vector, a vesicle, and a lipid nanoparticle.

62. The pharmaceutical composition of claim 61, wherein the viral vector is an adeno-associated virus.

63. A host cell (e.g., a mammalian cell, e.g., a human cell) comprising the template RNA or heterologous gene modifying system of any one of the preceding claims.

64. A method of making the template RNA of any one of claim 1-28, 46-51, 57, or 58, the method comprising synthesizing the template RNA by in vitro transcription (e.g., solid state synthesis) or by introducing a DNA encoding the template RNA into a host cell under conditions that allow for production of the template RNA.

65. A method for modifying a target site in the human TRAC gene and/or the B2M gene in a cell, the method comprising contacting the cell with the heterologous gene modifying system of any one of claims 29-56, or DNA encoding the same, thereby modifying the target site in the human TRAC gene and/or the B2M gene in a cell.

66. The heterologous gene modifying system or method of any one of claim 29-56 or 65 wherein insertion of the system into a target cell results in a mutation in that causes disruption of the TRAC gene and/or the B2M gene.

67. The heterologous gene modifying system or method of any of claim 29-56, 65, or 66, wherein installation of the mutation results in a loss of TRAC and/or the B2M expression.

68. The heterologous gene modifying system or method of any one of claim 29-56, or 65-67, wherein insertion of the system into a target cell results in a mutation in that disrupts splicing of the TRAC gene and/or the B2M gene.

69. The heterologous gene modifying system or method of claim of any one of claim 29-56, or 65-68, wherein mutation generates a stop codon (e.g., a premature stop codon) in the TRAC gene and/or the B2M gene.

70. The heterologous gene modifying system or method of any one of claim 29-56, or 65-69 wherein the mutation comprises insertion of between about 4 to 30 nucleotides in the TRAC gene, resulting in a frame shift in the TRAC gene and/or the B2M gene.

71. The heterologous gene modifying system or method of any one of claim 29-56, or 65-69, wherein the mutation results in a deletion of a region of the TRAC gene and/or the B2M gene.

72. The heterologous gene modifying system or method of any one of the preceding claims, wherein the mutation comprises a deletion of between about 4 and 30 nucleotides in the TRAC gene and/or the B2M gene.

73. The heterologous gene modifying system or method of any of the preceding claims, wherein the mutation is introduced at (i) a protospacer nick site or (ii) the PAM location associated with the protospacer

74. The heterologous gene modifying system or method of any of the preceding claims, wherein installation of the mutation occurs in at least 15% (e.g., 15%, 20%, 25%, 30%, 40%, 50%, 60%, 70%, 75%, or more) of target nucleic acids.

75. The heterologous gene modifying system or method of any of the preceding claims, wherein correction of the mutation occurs in at least 15% (e.g., 15%, 20%, 25%, 30%, 40%, 50%, 60%, 70%, 75%, or more) of target cells.

76. A system for modifying DNA comprising:
(a) a first gene modifying system comprising: (i) a retrotransposon gene modifying polypeptide or a nucleic acid (e.g., DNA or mRNA) encoding the retrotransposon gene modifying polypeptide, and (ii) a first template RNA (or DNA encoding the template RNA) comprising (1) a sequence that binds the retrotransposon gene modifying polypeptide and (2) a first heterologous object sequence; and
(b) a second system comprising: (i) a heterologous gene modifying polypeptide or a nucleic acid (e.g., DNA or mRNA) encoding the heterologous gene modifying polypeptide, (ii) a second template RNA (or DNA encoding the template RNA) comprising (1) a gRNA spacer, (2) a gRNA scaffold, (3) a heterologous object sequence, and (4) a primer binding site (PBS) sequence; and (iii) a third template RNA (or DNA encoding the template RNA) comprising (1) a gRNA spacer, (2) a gRNA scaffold, (3) a heterologous object sequence, and (4) a primer binding site (PBS) sequence.

77. The system of claim 76, wherein the second system comprises the heterologous gene modifying system of any of embodiments 29-56.

78. A system for modifying DNA comprising:
(a) a first gene modifying system comprising: (i) a retrotransposon gene modifying polypeptide or a nucleic acid (e.g., DNA or mRNA) encoding the retrotransposon gene modifying polypeptide, and (ii) a first template RNA (or DNA encoding the template RNA) comprising (1) a sequence that binds the retrotransposon gene modifying polypeptide and (2) a first heterologous object sequence (e.g., encoding a CAR); and
(b) a second system comprising: (i) a heterologous gene modifying polypeptide or a nucleic acid (e.g., DNA or mRNA) encoding the heterologous gene modifying polypeptide, and (ii) a second template RNA (or DNA encoding the template RNA) of any one of claim 1-28, 46-51, 57, or 58.

79. A system for modifying DNA comprising:
(a) a first gene modifying system comprising: (i) a retrotransposon gene modifying polypeptide or a nucleic acid (e.g., DNA or mRNA) encoding the retrotransposon gene modifying polypeptide, and (ii) a first template RNA (or DNA encoding the template RNA) comprising (1) a sequence that binds the retrotransposon gene modifying polypeptide and (2) a first heterologous object sequence (e.g., encoding a CAR); and
(b) a second system comprising: (i) a heterologous gene modifying polypeptide or a nucleic acid (e.g., DNA or mRNA) encoding the heterologous gene modifying polypeptide, (ii) a second template RNA (or DNA encoding the template RNA) of any one of claim 1-10, 21-28, 46-51, 57, or 58; and (iii) a third template RNA (or DNA encoding the template RNA) of any one of claim 11-28, 46-51, 57, or 58.

80. The system of any of claims 76-79, wherein the retrotransposon gene modifying polypeptide comprises an amino acid sequence of Table R1 or a sequence having no more than 10, 20, 30, 40, 50, 60, 70, 80, 90, or 100 amino acid differences thereto or a sequence having at least 80%, 85%, 90%, 95%, 97%, 98%, or 99% identity thereto, or a nucleic acid (e.g., DNA or mRNA) encoding the retrotransposon gene modifying polypeptide.

81. The system of any one of claims 76-80, wherein the retrotransposon gene modifying polypeptide comprises an amino acid sequence listed in Example 11 or Example 12 or a sequence having no more than 10, 20, 30, 40, 50, 60, 70, 80, 90, or 100 amino acid differences thereto or a sequence having at least 80%, 85%, 90%, 95%, 97%, 98%, or 99% identity thereto, or a nucleic acid (e.g., DNA or mRNA) encoding the retrotransposon gene modifying polypeptide.

82. The system of any of claims 76-81, wherein the sequence that binds the polypeptide comprises a 5′UTRᵣₑₜᵣₒ or a 3′ UTRᵣₑₜᵣₒ.

83. The system of claim 82, wherein the first template RNA comprises both of a 5′UTRᵣₑₜᵣₒ and a 3′ UTRᵣₑₜᵣₒ.

84. The system of claim 83, wherein the 5′UTRᵣₑₜᵣₒ and the 3′ UTRᵣₑₜᵣₒ comprise 5′ and 3′ sequences of Table R¹ or Example 11 or 12.

85. The system of any of claims 76-84, wherein the first heterologous object sequence encodes a chimeric antigen receptor (CAR), wherein the CAR comprises an antigen-binding domain, a transmembrane domain, a first intracellular signaling domain, and a second intracellular signaling domain.

86. The gene modifying system of any of claims 76-85, wherein the heterologous gene modifying polypeptide comprises:
a reverse transcriptase (RT) domain (e.g., an RT domain from a retrovirus, or a polypeptide domain having at least 75%, 80%, 85%, 90%, 95%, 96%, 97%, 98%, or 99% amino acids sequence identity thereto); and
a Cas domain that binds to the target DNA molecule and is heterologous to the RT domain (e.g., a Cas9 domain); and optionally, a linker disposed between the RT domain and the Cas domain.

87. The system of claim 86, wherein the RT domain comprises an amino acid sequence of Table 6, or a sequence having at least 70%, 80%, 85%, 90%, 95%, 97%, 98%, or 99% identity thereto.

88. The gene modifying system of claim 86 or 87, wherein the Cas domain comprises a Cas domain of Table 7 or Table 8A, or a sequence having at least 70%, 80%, 85%, 90%, 95%, 97%, 98%, or 99% identity thereto.

89. A method of modifying the genome of a mammalian cell, comprising contacting a population of cells with a system of any one of embodiments 76-88, thereby modifying the genome of a cell of the population.

90. The method of claim 89, wherein the first gene modifying system produces a first sequence alteration (e.g., an insertion) and the second system produces a second sequence alteration in the genome of the mammalian cell.

91. The method of claim 90, wherein at least 5%, 10%, or 20% of cells in the population comprise the first sequence alteration.

92. The method of any claim 90 or 91, wherein at least 10%, 20%, 30%, 40%, 50%, or 60% of cells in the population comprise the second sequence alteration.

93. The method of any one of claims 90-92, wherein at least 20%, 40%, 60%, 80% of cells that comprise the first sequence alteration also comprise the second sequence alteration.

94. A method for modifying the genome of a mammalian cell, the method comprising contacting a population of cells with a heterologous gene modifying system comprising:
(i) a heterologous gene modifying polypeptide or a nucleic acid (e.g., DNA or mRNA) encoding the heterologous gene modifying polypeptide,
(ii) a first template RNA (or DNA encoding the template RNA) comprising (1) a gRNA spacer, (2) a gRNA scaffold, (3) a heterologous object sequence, and (4) a primer binding site (PBS) sequence, wherein the first template RNA is configured to produce a first mutation; and
(iii) a second template RNA (or DNA encoding the template RNA) comprising (1) a gRNA spacer, (2) a gRNA scaffold, (3) a heterologous object sequence, and (4) a primer binding site (PBS) sequence, wherein the second template RNA is configured to produce a second mutation,
wherein contacting the population of cells with the system results in installation of the first and second mutations (e.g., insertions) in at least 40%, 50%, 60%, 70%, 75% of said cells.

95. The method of claim 94, wherein the first template RNA comprises a gRNA spacer that is complementary to portion of the human TRAC gene (e.g., comprises a gRNA spacer sequence of Table 1A, 2A, or 3A).

96. The method of claim 94 or 95, wherein the second template RNA comprises a gRNA spacer that is complementary to portion of the human B2M gene (e.g., comprises a gRNA spacer sequence of Table 1B, 2B, or 3B).

97. The method of any one of claims 94-96, wherein the modifying does not result in a translocation event.

98. The method of any one of claim 65-75 or 89-97, wherein the modifying does not result in a translocation event.

99. The method of any of the preceding claims, wherein the cell is a mammalian cell, such as a human cell.

100. The method of claim 99, wherein the mammalian cell is an immune effector cell.

101. The method of claim 99 or 100, wherein the mammalian cell is a T cell, e.g., a primary T cell.

102. The method of any one of the preceding claims, wherein the subject is a human.

103. The method of any of the preceding claims, wherein the contacting occurs ex vivo, e.g., wherein the cell's or subject's DNA is modified ex vivo.

104. The method of any of the preceding claims, wherein the contacting occurs in vivo, e.g., wherein the cell's or subject's DNA is modified in vivo.

105. The method of any of the preceding claims, wherein contacting the cell or the subject with the system comprises contacting the cell or a cell within the subject with a nucleic acid (e.g., DNA or RNA) encoding the heterologous gene modifying polypeptide under conditions that allow for production of the heterologous gene modifying polypeptide.
